(19) United States
(12) Patent Application Publication
(10) Pub. No.: US 2025/0026813 A1
(43) Pub. Date: Jan. 23, 2025

(54) ANTIBODIES AGAINST SARS-COV-2 AND USES THEREOF

(71) Applicant: Kymab Limited, Cambridge (GB)

(72) Inventors: Paul Kellam, Cambridge (GB); Anne Palser, Cambridge (GB); Volker Germaschewski, Cambridge (GB); Simon James Watson, Cambridge (GB); Benjamin David Grimshaw, Cambridge (GB); Spela Binter, Cambridge (GB); Jaroslaw Michal Szary, Cambridge (GB); Margot Billaud, Cambridge (GB); Robert Rowlands, Cambridge (GB); Aishwarya Krishna, Cambridge (GB); Huan-Chun Lin, Cambridge (GB); Cheng-Yuan Yang, Cambridge (GB); Li-Ying Liou, Cambridge (GB)

(21) Appl. No.: 18/250,716

(22) PCT Filed: Oct. 27, 2021
(86) PCT No.: PCT/EP2021/079901

§ 371 Date: Apr. 26, 2023

(30) Foreign Application Priority Data
Oct. 27, 2020 (GB) 2017058.5

(51) Int. Cl. C07K 16/10

(52) U.S. Cl. CPC C07K 16/1003; C07K 2317/24; C07K 2317/76; C07K 2317/92

(57) ABSTRACT

The present invention relates to antibodies that are specific for SARS-CoV-2. The present invention also provides methods of treatment, uses, pharmaceutical compositions and kits comprising the antibodies.

### FIELD OF THE INVENTION

The present invention relates to antibodies that bind the spike protein (S) of Severe acute respiratory syndrome coronavirus 2 (SARS-CoV-2) the strain of coronavirus that causes pandemic coronavirus infectious disease 2019 (COVID-19), and their use in diagnosis, prevention and treatment of SARS-CoV-2 related diseases, particularly COVID-19.

### BACKGROUND

The new pandemic coronavirus, Severe Acute Respiratory Syndrome Coronavirus 2 (SARS-CoV-2) emerged into humans in China sometime between October to November 2019, and the disease Coronavirus Infectious Disease-2019 (COVID-19) was identified in China in December 2019. SARS-CoV-2 causes COVID-19 in humans. Although an asymptomatic or mild infection in many people, the virus can cause severe respiratory disease and death in a significant number of people, especially in the elderly and in those with underlying co-morbidities. Initial recognition of a new human pneumonia, negative for all known human respiratory pathogens, occurred by recognition of related symptoms in hospitals in Wuhan, China, together with a common epidemiological link to a ‘wet market’ in Wuhan. Rapid identification of a new coronavirus genome, and the development of specific and sensitive virus detection diagnostics lead to the recognition of the explosive spread of the virus in Wuhan, followed by other regions of China and surrounding neighbouring countries.

As of 2 Sep. 2020, SARS-CoV-2 has spread throughout the world infecting an estimated ˜25 million people, resulting in 861,000 deaths, yielding a nominal infection fatality rate (IFR) of ˜3%. This number is likely an overestimate, due to hidden, asymptomatic or mild, and non-diagnosed cases. Although the true infection fatality rate is difficult to calculate, current estimates range from 0.1-1%.

COVID-19 can be a mild to moderate self-limiting disease in about 80% of infected people. These people experience symptoms of fever, myalgia, dry persistent cough and shortness of breath. This disease course is usually complete in 7-10 days, but recovery to full health may take longer. However, in −20% of cases, a more aggressive and severe disease occurs, either with rapid progression from symptom onset or a rapid decline from the initial 7-10 days of moderate infection when recovery was apparently beginning. Such serious disease is associated with lymphopenia, elevated troponin and D-dimer levels in the blood and both consolidated or diffuse bilateral (both lungs) ‘ground glass’ pneumonia. Many of these cases require breathing support and ˜20-25% of the serious cases become critically ill. The IFR for critical patients is historically ˜40%-50%, consistent with the overall IFR of 1-2 people per 100 diagnosed as infected. Recently, the long-term consequences of infection by SARS-CoV-2 are becoming apparent, so-called Long-COVID, characterised by recurring symptoms experienced by patients, regardless of whether they were hospitalised, affecting the respiratory system, the brain, cardiovascular system and heart, the kidneys, the gut, the liver, and the skin. The symptoms can range in intensity and duration and are estimated to affect 10-15% of people recovered from their initial infection.

SARS-CoV-2 is a beta coronavirus, closely related to the 2002 SARS-CoV. SARS-CoV of 2002 infected 8,098 people causing 774 deaths (IFR 9.5%). SARS-CoV has not been seen since 2004 and was controlled by aggressive infection control measures and quarantining.

The more distantly related beta coronavirus Middle East Respiratory Syndrome coronavirus (MERS-CoV), emerged in Saudi Arabia in 2012. Infections with MERS-CoV continue to occur in the Middle East, as a result of ongoing zoonotic (animal to human) infection from camels, the reservoir animal species, to humans. MERS-CoV as an IRF of 35% and since April 2012, more than 2,400 cases of Middle East respiratory syndrome coronavirus (MERS-CoV) have been detected in 27 countries.

SARS-CoV-2, SARS-CoV and MERS-CoV represent epidemic/pandemic coronavirus. However, four endemic coronaviruses (NL63, 229E, OC43 and HKU1) also infect humans in childhood and throughout adult life, causing mild upper respiratory tract infections but occasionally causing severe life-threatening disease. Current opinion suggests SARS-CoV-2 is on the path to becoming the 5^{th} seasonal endemic human coronavirus.

The major components of the SARS-CoV-2 virus particle are its externally-oriented spike protein and its virus RNA genome which is wrapped in nucleocapsid proteins. The spike protein is a trimeric virus protein embedded in the lipid membrane of the virus particle. Viral infection is initiated when the spike protein binds to the cellular Angiotensin Converting Enzyme 2 (ACE2) receptor resulting in internalisation of the virus into the cell. The most likely path for the viruses into the cell is via receptor mediated cellular uptake into the endosomal pathway of the cell, where the virus encounters an activating cellular protease (cathepsin). This cell enzyme proteolytically cleaves the spike triggering a membrane fusion event between the virus lipid envelope and the lipid shell of the endosome, resulting in the entry of the virus genome into the cell cytoplasm. Once in the cell cytoplasm, the virus begins the process of making multiple new copies of its genome, whilst simultaneously making new copies of virus proteins. These new proteins and genomes assemble into virus particles where they bud into a structure of the cell called the endoplasmic reticulum, which pinches off into small vesicles for transport to the cell surface where new virus particles are released to the outside of the infected cell. These new virus particles are then free to infect more cells. In addition, the presence of TMPRSS2 on the cell surface can proteolytically cleave the spike after ACE-2 binding triggering a membrane fusion at the plasma membrane.

The virus life cycle provides points of chemotherapeutic intervention. Currently the only directly acting antiviral (DAA) is remdesivir (Gilead), an adenosine nucleotide analogue which inhibits the virus RNA dependent RNA polymerase, the enzyme that replicates the virus genome. The clinical utility of remdesivir is not clear and experience with antiviral drugs to other respiratory pathogens such as influenza A virus and respiratory syncytial virus (RSV), suggest that drugs like remdesivir have restricted use, being optimal in efficacy if taken very early in infection. This is almost impossible to achieve in practice with SARS-CoV-2 as the person is not symptomatic at the time of infection when this drug will have greatest impact. Therefore, potentially remdesivir's activity profile will prove to be similar to the influenza A drugs, oseltamivir and zanamivir, where their effect on severe disease is limited, and the chemoprophylaxis of case contacts is limited without mass deployment into people's homes, something unlikely to happen with a new drug like remdesivir, with a limited safety profile. Other small molecule antiviral drugs are expected to emerge as research proceeds. In particular, the virus protease is an attractive target. However, such inhibitors are all likely to suffer from a restricted optimal efficacy window, which in most case occurs before or at the time of symptom onset.

Clinical management of serious disease is the subject of ongoing and planned clinical trials, which have already shown a number of failures and one success. For example, hydroxychloroquine, an anti-malarial drug similar to chloroquine, has failed to show clinical effect in a number of clinical trials. In addition, the anti-IL6 receptor antibody tocilizumab (Genentech), used for the treatment of cytokine release syndrome (CRS) and of sarilumab (anti-IL6 receptor antibody, Regeneron) have failed to show clinical efficacy. However, dexamethasone, a steroidal anti-inflammatory drug has shown clinical efficacy in treating severe COVID-19.

Changes in the critical care of patients will also have effects, either through the scaling of access to ventilators (surge capacity) or the provision of continuous positive airway pressure (CPAP) as a form of respiratory support. CPAP is reported to have a positive clinical effect on the ability of people to survive severe COVID-19 without the need for full ventilation. Further, the proning of ventilated patients improves the clinical course of disease.

The normal course of an infectious disease process is:
1. Infection,
2. Virus replication in the body with the innate immune response providing the system that non-specifically attempts to limit early uncontrolled replication,
3. Initiation of the adaptive immune response that drives within the body the production of B cells and T cells specific to the new virus infection,
4. Virus specific B cells expand in the body and secrete antibodies, their effector molecules.
5. Virus specific T cells expand in the body to kill infected cells,

Infection by SARS-CoV-2 results in an adaptive immune response, with induction of antibodies and T cells which specifically bind virus proteins. The adaptive immune system begins to have a meaningful effect on controlling the infection within about 7-10 days from the point of virus replication in the body, reaching its peak activity around 10-14 days.

### Spike Protein

The spike protein is a trimer of three monomers. Each monomer is about 180 kDa, and contains two subunits, S1 and S2, mediating attachment and membrane fusion, respectively. The N- and C-terminal portions of S1 fold as independent domains, the N-terminal domain (NTD), the receptor binding domain (RBD) and the C-terminal domain (C-domain) which associates with the S2 subunit. FIG. 1 depicts a single monomer of the trimeric spike protein. The monomer is translated as a single polypeptide which is proteolytically cleaved into the subunits S1 and S2 which non-covalently associate. The amino acid sequence of the wild-type SARS-CoV-2 spike protein is provided in FIG. 2A and the amino acid sequence of the SARS-CoV-2 spike protein containing double proline (PP) mutations (K986 and V987) (as compared to wild-type) is provided in FIG. 2B.

Coronavirus S proteins are typical class I viral fusion proteins, and protease cleavage is required for activation of the fusion potential of the S protein. In a two-step sequential protease cleavage model, as is thought to occur for SARS-CoV-2, a priming cleavage occurs between S1 and S2 and activating cleavage occurs at the S2′ cleavage site. Further the RBD of each trimer is dynamic, existing in either an ‘UP’ or ‘DOWN’ configuration. The UP state is required for ACE2 receptor binding. Therefore, a trimeric spike can exist with all 3 RBD DOWN, 2 DOWN and 1 UP, 1 DOWN and 2 UP or all 3 RBDs UP. Each RBD in the trimer is able to bind ACE2 in its UP state and initiate infection.

### SUMMARY OF THE INVENTION

We have discovered monoclonal antibodies that bind the spike protein of SARS-CoV-2 and have properties suitable for development as medicaments for treating or preventing viral infection. Monoclonal antibodies of the invention demonstrate a combination of advantageous properties, including binding location on the spike protein of SARS-CoV-2, binding affinity to the spike protein of SARS-CoV-2 and/or potency of neutralisation of SARS-CoV-2. We demonstrate herein that exemplary antibodies neutralise SARS-CoV-2, particularly in vitro in pseudovirus assays and/or in live virus assays. Neutralising antibodies described herein, i.e. antibodies that neutralise SARS-CoV-2, will generally be understood to inhibit or prevent SARS-CoV-2 entering cells expressing the ACE2 receptor, e.g. lung epithelial cells. Antibodies might neutralise SARS-CoV-2 e.g. by competing with ACE2 for binding to SARS-CoV-2.

We further demonstrate herein that exemplary antibodies neutralise SARS-CoV-2 and specifically bind to the receptor binding domain (RBD) of the S1 subunit of SARS-CoV-2. Such antibodies may or may not compete with ACE2 for binding to SARS-CoV-2 and thus may or may not directly inhibit binding of SARS-CoV-2 to its receptor ACE2.

We further demonstrate herein that exemplary antibodies preferentially bind to the trimer form of the SARS-CoV-2 spike protein over the isolated RBD domain, S1 subunit and S2 subunit of the SARS-CoV-2 spike protein. Such antibodies generally do not compete with ACE2 for binding to SARS-CoV-2.

We further demonstrate herein that exemplary antibodies neutralise SARS-CoV-2 and specifically bind to the S2 subunit of SARS-CoV-2. Such antibodies generally do not compete with ACE2 for binding to SARS-CoV-2.

We further demonstrate herein that exemplary antibodies neutralise SARS-CoV-2 and specifically bind to the N-terminal domain (NTD) of the S1 subunit of the SARS-CoV-2 spike protein. Such antibodies generally do not compete with ACE2 for binding to SARS-CoV-2.

We further demonstrate herein that exemplary antibodies have high affinity (such as a KD of 10-9 M or lower or even a KD of 5×10⁻¹⁰ M, 1×10⁻¹⁰ M (0.1 nM) or lower) for SARS-CoV-2, particularly when measured using a surface plasmon resonance (SPR) assay (e.g. a Biacore SPR assay).

We demonstrate herein that exemplary antibodies neutralise SARS-CoV-2 with high potency (such as with an IC50 of lnM or lower, an IC50 of 100 pM or lower, an IC50 of 50 pM or lower, an IC50 of 10 pM or lower, or even an IC50 of 5 pM or lower) particularly in vitro in pseudovirus assays.

An aim of the present invention is to reduce the incidence of severe and critical disease and death through the administration of such monoclonal antibodies.

Administration of monoclonal antibodies of the invention may be used either as a prophylactic or as a therapeutic. Monoclonal antibodies of the invention may also be used in diagnosis.

### Group A—Competing RBD Binders:

In a first aspect, the present invention provides an antibody that specifically binds to the receptor binding domain (RBD) of the SARS-CoV-2 spike protein, wherein the antibody competes for binding to the SARS-CoV2 spike protein with the human ACE2 receptor.

In one embodiment, the antibody binds the isolated RBD of the SARS-CoV-2 spike protein with a KD of 10⁻⁹ M or lower (e.g. as measured by surface plasmon resonance (SPR)).

In one embodiment, the antibody neutralises SARS-CoV-2 with an IC50 of 50 pM or lower (e.g. as measured in a pseudovirus neutralisation assay).

In one embodiment, the antibody binds the isolated RBD of the SARS-CoV-2 spike protein with a KD of 10-9 M or lower (e.g. as measured by surface plasmon resonance (SPR)) and the antibody neutralises SARS-CoV2 with an IC50 of 50 pM or lower (e.g. as measured in a pseudovirus neutralisation assay).

In one embodiment, the antibody comprises a variable heavy (VH) domain sequence comprising complementarity determining regions (CDRs) HCDR1, HCDR2 and HCDR3, and a variable light (VL) domain sequence comprising complementarity determining regions LCDR1, LCDR2 and LCDR3, wherein the HCDR3 is the HCDR3 of antibody IMPI-029, IMPI-056, IMPI-005, IMPI-012, IMPI-052, IMPI-002, IMPI-041, IMPI-036, IMPI-055, IMPI-054, IMPI-042, IMPI-021, IMPI-004, IMPI-047, IMPI-017, IMPI-059, IMPI-0060, IMPI-006, IMPI-037, or IMPI-028.

In one embodiment, the antibody comprises a variable heavy (VH) domain sequence comprising complementarity determining regions (CDRs) HCDR1, HCDR2 and HCDR3, and a variable light (VL) domain sequence comprising complementarity determining regions LCDR1, LCDR2 and LCDR3, wherein HCDR3 is the HCDR3 of antibody IMPI-029, IMPI-056, IMPI-005, IMPI-012, IMPI-052, IMPI-002, IMPI-041, IMPI-036, IMPI-055, IMPI-054, IMPI-042, IMPI-021, IMPI-004, IMPI-047, IMPI-017, IMPI-059, or IMPI-028.

In one embodiment, the antibody binds the isolated RBD of the SARS-CoV-2 spike protein with a KD of 10-9 M or lower (e.g. as measured by surface plasmon resonance (SPR)) and HCDR3 is the HCDR3 of antibody IMPI-004, IMPI-006, IMPI-029, IMPI-056, IMPI-055, or IMPI-059 or an antibody in the same cluster as one of these antibodies.

In one embodiment, the antibody binds the isolated RBD of the SARS-CoV-2 spike protein with a KD of 10-9 M or lower (e.g. as measured by surface plasmon resonance (SPR)) and HCDR3 is the HCDR3 of antibody IMPI-004, IMPI-006, IMPI-029, IMPI-056, IMPI-055, or IMPI-059.

In one embodiment, the antibody binds the isolated RBD of the SARS-CoV-2 spike protein with a KD of 10-9 M or lower (e.g. as measured by surface plasmon resonance (SPR)) and HCDR3 is the HCDR3 of antibody IMPI-004, IMPI-029, IMPI-056, IMPI-055, or IMPI-059 or an antibody in the same cluster as one of these antibodies.

In one embodiment, the antibody binds the isolated RBD of the SARS-CoV-2 spike protein with a KD of 10-9 M or lower (e.g. as measured by surface plasmon resonance (SPR)) and HCDR3 is the HCDR3 of antibody IMPI-004, IMPI-029, IMPI-056, IMPI-055, or IMPI-059.

In one embodiment, the antibody neutralises SARS-CoV-2 with an IC50 of 50 pM or lower (e.g. as measured in a pseudovirus neutralisation assay) and HCDR3 is the HCDR3 of antibody IMPI-004, IMPI-029, IMPI-055, or IMPI-059, or IMPI-017 or an antibody in the same cluster as one of these antibodies.

In one embodiment, the antibody neutralises SARS-CoV-2 with an IC50 of 50 pM or lower (e.g. as measured in a pseudovirus neutralisation assay) and HCDR3 is the HCDR3 of antibody IMPI-004, IMPI-029, IMPI-055, or IMPI-059, or IMPI-017.

In one embodiment, the antibody binds the isolated RBD of the SARS-CoV-2 spike protein with a KD of 10-9 M or lower (e.g. as measured by surface plasmon resonance (SPR)) and the antibody neutralises SARS-CoV-2 with an IC50 of 50 pM or lower (e.g. as measured in a pseudovirus neutralisation assay) and HCDR3 is the HCDR3 of antibody IMPI-004, IMPI-029, IMPI-055, or IMPI-059 or an antibody in the same cluster as one of these antibodies.

In one embodiment, the antibody binds the isolated RBD of the SARS-CoV-2 spike protein with a KD of 10-9 M or lower (e.g. as measured by surface plasmon resonance (SPR)) and the antibody neutralises SARS-CoV-2 with an IC50 of 50 pM or lower (e.g. as measured in a pseudovirus neutralisation assay) and HCDR3 is the HCDR3 of antibody IMPI-004, IMPI-029, or IMPI-055.

In one embodiment, HCDR3 is the HCDR3 of antibody IMPI-029.

In one embodiment, HCDR3 is the HCDR3 of antibody IMPI-056.

In one embodiment, HCDR3 is the HCDR3 of antibody IMPI-005.

In one embodiment, HCDR3 is the HCDR3 of antibody IMPI-012.

In one embodiment, HCDR3 is the HCDR3 of antibody IMPI-052.

In one embodiment, HCDR3 is the HCDR3 of antibody IMPI-002.

In one embodiment, HCDR3 is the HCDR3 of antibody IMPI-041.

In one embodiment, HCDR3 is the HCDR3 of antibody IMPI-036.

In one embodiment, HCDR3 is the HCDR3 of antibody IMPI-055.

In one embodiment, HCDR3 is the HCDR3 of antibody IMPI-054.

In one embodiment, HCDR3 is the HCDR3 of antibody IMPI-042.

In one embodiment, HCDR3 is the HCDR3 of antibody IMPI-021.

In one embodiment, HCDR3 is the HCDR3 of antibody IMPI-004.

In one embodiment, HCDR3 is the HCDR3 of antibody IMPI-047.

In one embodiment, HCDR3 is the HCDR3 of antibody IMPI-017.

In one embodiment, HCDR3 is the HCDR3 of antibody IMPI-059.

In one embodiment, HCDR3 is the HCDR3 of antibody IMPI-060.

In one embodiment, HCDR3 is the HCDR3 of antibody IMPI-006.

In one embodiment, HCDR3 is the HCDR3 of antibody IMPI-037.

In one embodiment, HCDR3 is the HCDR3 of antibody IMPI-028.

In one embodiment, the present invention provides an anti-SARS-CoV-2 antibody,

wherein the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3,
wherein the CDRs are those of antibody IMPI-029, IMPI-056, IMPI-005, IMPI-012, IMPI-052, IMPI-002, IMPI-041, IMPI-036, IMPI-055, IMPI-054, IMPI-042, IMPI-021, IMPI-004, IMPI-047, IMPI-017, IMPI-059, IMPI-060, IMPI-006, IMPI-037, or IMPI-028.

In one embodiment, the present invention provides an anti-SARS-CoV-2 antibody,

wherein the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3,
wherein the CDRs are those of antibody IMPI-029, IMPI-056, IMPI-005, IMPI-012, IMPI-052, IMPI-002, IMPI-041, IMPI-036, IMPI-055, IMPI-054, IMPI-042, IMPI-021, IMPI-004, IMPI-047, IMPI-017, IMPI-059, or IMPI-028.

In one embodiment, the present invention provides an anti-SARS-CoV-2 antibody which competes for binding to the SARS-CoV-2 spike protein with the human ACE2 receptor,

wherein the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3,
wherein the CDRs are those of antibody IMPI-029, IMPI-056, IMPI-005, IMPI-012, IMPI-052, IMPI-002, IMPI-041, IMPI-036, IMPI-055, IMPI-054, IMPI-042, IMPI-021, IMPI-004, IMPI-047, IMPI-017, IMPI-059, IMPI-060, IMPI-006, IMPI-037, or IMPI-028.

In one embodiment, the present invention provides an anti-SARS-CoV-2 antibody which competes for binding to the SARS-CoV-2 spike protein with the human ACE2 receptor,

wherein the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3,
wherein the CDRs are those of antibody IMPI-029, IMPI-056, IMPI-005, IMPI-012, IMPI-052, IMPI-002, IMPI-041, IMPI-036, IMPI-055, IMPI-054, IMPI-042, IMPI-021, IMPI-004, IMPI-047, IMPI-017, IMPI-059, or IMPI-028.

In one embodiment of the first aspect, the CDRs are those of antibody IMPI-029, IMPI-056, IMPI-005, IMPI-012, IMPI-052, IMPI-002, IMPI-041, IMPI-036, IMPI-055, IMPI-054, IMPI-042, IMPI-021, IMPI-004, IMPI-047, IMPI-017, IMPI-059, IMPI-060, IMPI-006, IMPI-037, or IMPI-028.

In one embodiment of the first aspect, the CDRs are those of antibody IMPI-029, IMPI-056, IMPI-005, IMPI-012, IMPI-052, IMPI-002, IMPI-041, IMPI-036, IMPI-055, IMPI-054, IMPI-042, IMPI-021, IMPI-004, IMPI-047, IMPI-017, IMPI-059, or IMPI-028.

In one embodiment, the antibody binds the isolated RBD of the SARS-CoV-2 spike protein with a KD of 10-9 M or lower (e.g. as measured by surface plasmon resonance (SPR)) and the CDRs are those of antibody IMPI-004, IMPI-029, IMPI-056, IMPI-055, IMPI-006, or IMPI-059 or an antibody in the same cluster as one of these antibodies.

In one embodiment, the antibody binds the isolated RBD of the SARS-CoV-2 spike protein with a KD of 10-9 M or lower (e.g. as measured by surface plasmon resonance (SPR)) and the CDRs are those of antibody IMPI-004, IMPI-029, IMPI-056, IMPI-055, IMPI-006, or IMPI-059.

In one embodiment, the antibody binds the isolated RBD of the SARS-CoV-2 spike protein with a KD of 10-9 M or lower (e.g. as measured by surface plasmon resonance (SPR)) and the CDRs are those of antibody IMPI-004, IMPI-029, IMPI-056, IMPI-055, or IMPI-059 or an antibody in the same cluster as one of these antibodies.

In one embodiment, the antibody binds the isolated RBD of the SARS-CoV-2 spike protein with a KD of 10-9 M or lower (e.g. as measured by surface plasmon resonance (SPR)) and the CDRs are those of antibody IMPI-004, IMPI-029, IMPI-056, IMPI-055, or IMPI-059.

In one embodiment, the antibody neutralises SARS-CoV-2 with an IC50 of 50 pM or lower (e.g. as measured in a pseudovirus neutralisation assay) and the CDRs are those of antibody IMPI-004, IMPI-029, IMPI-055, or IMPI-059, or IMPI-017 or an antibody in the same cluster as one of these antibodies.

In one embodiment, the antibody neutralises SARS-CoV-2 with an IC50 of 50 pM or lower (e.g. as measured in a pseudovirus neutralisation assay) and the CDRs are those of antibody IMPI-004, IMPI-029, IMPI-055, or IMPI-059, or IMPI-017.

In one embodiment, the antibody binds the isolated RBD of the SARS-CoV-2 spike protein with a KD of 10-9 M or lower (e.g. as measured by surface plasmon resonance (SPR)) and the antibody neutralises SARS-CoV-2 with an IC50 of 50 pM or lower (e.g. as measured in a pseudovirus neutralisation assay) and the CDRs are those of antibody IMPI-004, IMPI-029, IMPI-055, or IMPI-059 or an antibody in the same cluster as one of these antibodies.

In one embodiment, the antibody binds the isolated RBD of the SARS-CoV-2 spike protein with a KD of 10-9 M or lower (e.g. as measured by surface plasmon resonance (SPR)) and the antibody neutralises SARS-CoV-2 with an IC50 of 50 pM or lower (e.g. as measured in a pseudovirus neutralisation assay) and the CDRs are those of antibody IMPI-004, IMPI-029, IMPI-055, or IMPI-059.

In one embodiment, the antibody has the CDRs of antibody IMPI-029.

In one embodiment, the antibody has the CDRs of antibody IMPI-056.

In one embodiment, the antibody has the CDRs of antibody IMPI-005.

In one embodiment, the antibody has the CDRs of antibody IMPI-012.

In one embodiment, the antibody has the CDRs of antibody IMPI-052.

In one embodiment, the antibody has the CDRs of antibody IMPI-002.

In one embodiment, the antibody has the CDRs of antibody IMPI-041.

In one embodiment, the antibody has the CDRs of antibody IMPI-036.

In one embodiment, the antibody has the CDRs of antibody IMPI-055.

In one embodiment, the antibody has the CDRs of antibody IMPI-054.

In one embodiment, the antibody has the CDRs of antibody IMPI-042.

In one embodiment, the antibody has the CDRs of antibody IMPI-021.

In one embodiment, the antibody has the CDRs of antibody IMPI-004.

In one embodiment, the antibody has the CDRs of antibody IMPI-047.

In one embodiment, the antibody has the CDRs of antibody IMPI-017.

In one embodiment, the antibody has the CDRs of antibody IMPI-059.

In one embodiment, the antibody has the CDRs of antibody IMPI-060.

In one embodiment, the antibody has the CDRs of antibody IMPI-006.

In one embodiment, the antibody has the CDRs of antibody IMPI-037.

In one embodiment, the antibody has the CDRs of antibody IMPI-028.

In one embodiment, the antibody comprises a variable heavy (VH) domain sequence and a variable light (VL) domain sequence and wherein the variable heavy (VH) domain and variable light (VL) domain sequences respectively comprise the variable heavy (VH) domain and variable light (VL) domain sequences of antibody IMPI-029, IMPI-056, IMPI-005, IMPI-012, IMPI-052, IMPI-002, IMPI-041, IMPI-036, IMPI-055, IMPI-054, IMPI-042, IMPI-021, IMPI-004, IMPI-047, IMPI-017, IMPI-059, IMPI-060, IMPI-006, IMPI-037, or IMPI-028, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

In one embodiment, the antibody comprises a variable heavy (VH) domain sequence and a variable light (VL) domain sequence and wherein the variable heavy (VH) domain and variable light (VL) domain sequences respectively comprise the variable heavy (VH) domain and variable light (VL) domain sequences of antibody IMPI-029, IMPI-056, IMPI-005, IMPI-012, IMPI-052, IMPI-002, IMPI-041, IMPI-036, IMPI-055, IMPI-054, IMPI-042, IMPI-021, IMPI-004, IMPI-047, IMPI-017, IMPI-059, or IMPI-028, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

In one embodiment, the antibody binds the isolated RBD of the SARS-CoV-2 spike protein with a KD of 10-9 M or lower (e.g. as measured by surface plasmon resonance (SPR)) and the variable heavy (VH) domain and variable light (VL) domain sequences respectively comprise the variable heavy (VH) domain and variable light (VL) domain sequences of antibody IMPI-004, IMPI-029, IMPI-056, IMPI-055, IMPI-006 or IMPI-059 or an antibody in the same cluster as one of these antibodies, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

In one embodiment, the antibody binds the isolated RBD of the SARS-CoV-2 spike protein with a KD of 10-9 M or lower (e.g. as measured by surface plasmon resonance (SPR)) and the variable heavy (VH) domain and variable light (VL) domain sequences respectively comprise the variable heavy (VH) domain and variable light (VL) domain sequences of antibody IMPI-004, IMPI-029, IMPI-056, IMPI-055, IMPI-006 or IMPI-059, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

In one embodiment, the antibody binds the isolated RBD of the SARS-CoV-2 spike protein with a KD of 10-9 M or lower (e.g. as measured by surface plasmon resonance (SPR)) and the variable heavy (VH) domain and variable light (VL) domain sequences respectively comprise the variable heavy (VH) domain and variable light (VL) domain sequences of antibody IMPI-004, IMPI-029, IMPI-056, IMPI-055, or IMPI-059 or an antibody in the same cluster as one of these antibodies, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

In one embodiment, the antibody binds the isolated RBD of the SARS-CoV-2 spike protein with a KD of 10-9 M or lower (e.g. as measured by surface plasmon resonance (SPR)) and the variable heavy (VH) domain and variable light (VL) domain sequences respectively comprise the variable heavy (VH) domain and variable light (VL) domain sequences of antibody IMPI-004, IMPI-029, IMPI-056, IMPI-055, or IMPI-059, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

In one embodiment, the antibody neutralises SARS-CoV-2 with an IC50 of 50 pM or lower (e.g. as measured in a pseudovirus neutralisation assay) and the variable heavy (VH) domain and variable light (VL) domain sequences respectively comprise the variable heavy (VH) domain and variable light (VL) domain sequences of antibody IMPI-004, IMPI-029, IMPI-055, or IMPI-059, or IMPI-017 or an antibody in the same cluster as one of these antibodies, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

In one embodiment, the antibody neutralises SARS-CoV-2 with an IC50 of 50 pM or lower (e.g. as measured in a pseudovirus neutralisation assay) and the variable heavy (VH) domain and variable light (VL) domain sequences respectively comprise the variable heavy (VH) domain and variable light (VL) domain sequences of antibody IMPI-004, IMPI-029, IMPI-055, or IMPI-059, or IMPI-017, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

In one embodiment, the antibody binds the isolated RBD of the SARS-CoV-2 spike protein with a KD of 10-9 M or lower (e.g. as measured by surface plasmon resonance (SPR)) and the antibody neutralises SARS-CoV2 with an IC50 of 50 pM or lower (e.g. as measured in a pseudovirus neutralisation assay) and the variable heavy (VH) domain and variable light (VL) domain sequences respectively comprise the variable heavy (VH) domain and variable light (VL) domain sequences of antibody IMPI-004, IMPI-029, IMPI-055, or IMPI-059 or an antibody in the same cluster as one of these antibodies, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

In one embodiment, the antibody binds the isolated RBD of the SARS-CoV-2 spike protein with a KD of 10-9 M or lower (e.g. as measured by surface plasmon resonance (SPR)) and the antibody neutralises SARS-CoV2 with an IC50 of 50 pM or lower (e.g. as measured in a pseudovirus neutralisation assay) and the variable heavy (VH) domain and variable light (VL) domain sequences respectively comprise the variable heavy (VH) domain and variable light (VL) domain sequences of antibody IMPI-004, IMPI-029, IMPI-055, or IMPI-059, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

In one embodiment, the antibody comprises a variable heavy (VH) domain sequence of antibody IMPI-029, optionally with 1, 2, 3, 4 or 5 amino acid alterations outside the complementarity determining regions (CDRs), and a variable light (VL) domain sequence of antibody IMPI-029, optionally with 1, 2, 3, 4 or 5 amino acid alterations outside the complementarity determining regions (CDRs).

In one embodiment, the antibody comprises a variable heavy (VH) domain sequence of antibody IMPI-056, optionally with 1, 2, 3, 4 or 5 amino acid alterations outside the complementarity determining regions (CDRs), and a variable light (VL) domain sequence of antibody IMPI-056, optionally with 1, 2, 3, 4 or 5 amino acid alterations outside the complementarity determining regions (CDRs).

In one embodiment, the antibody comprises a variable heavy (VH) domain sequence of antibody IMPI-005, optionally with 1, 2, 3, 4 or 5 amino acid alterations outside the complementarity determining regions (CDRs), and a variable light (VL) domain sequence of antibody IMPI-005, optionally with 1, 2, 3, 4 or 5 amino acid alterations outside the complementarity determining regions (CDRs).

In one embodiment, the antibody comprises a variable heavy (VH) domain sequence of antibody IMPI-012, optionally with 1, 2, 3, 4 or 5 amino acid alterations outside the complementarity determining regions (CDRs), and a variable light (VL) domain sequence of antibody IMPI-012, optionally with 1, 2, 3, 4 or 5 amino acid alterations outside the complementarity determining regions (CDRs).

In one embodiment, the antibody comprises a variable heavy (VH) domain sequence of antibody IMPI-052, optionally with 1, 2, 3, 4 or 5 amino acid alterations outside the complementarity determining regions (CDRs), and a variable light (VL) domain sequence of antibody IMPI-052, optionally with 1, 2, 3, 4 or 5 amino acid alterations outside the complementarity determining regions (CDRs).

In one embodiment, the antibody comprises a variable heavy (VH) domain sequence of antibody IMPI-002, optionally with 1, 2, 3, 4 or 5 amino acid alterations outside the complementarity determining regions (CDRs), and a variable light (VL) domain sequence of antibody IMPI-002, optionally with 1, 2, 3, 4 or 5 amino acid alterations outside the complementarity determining regions (CDRs).

In one embodiment, the antibody comprises a variable heavy (VH) domain sequence of antibody IMPI-041, optionally with 1, 2, 3, 4 or 5 amino acid alterations outside the complementarity determining regions (CDRs), and a variable light (VL) domain sequence of antibody IMPI-041, optionally with 1, 2, 3, 4 or 5 amino acid alterations outside the complementarity determining regions (CDRs).

In one embodiment, the antibody comprises a variable heavy (VH) domain sequence of antibody IMPI-036, optionally with 1, 2, 3, 4 or 5 amino acid alterations outside the complementarity determining regions (CDRs), and a variable light (VL) domain sequence of antibody IMPI-036, optionally with 1, 2, 3, 4 or 5 amino acid alterations outside the complementarity determining regions (CDRs).

In one embodiment, the antibody comprises a variable heavy (VH) domain sequence of antibody IMPI-055, optionally with 1, 2, 3, 4 or 5 amino acid alterations outside the complementarity determining regions (CDRs), and a variable light (VL) domain sequence of antibody IMPI-055, optionally with 1, 2, 3, 4 or 5 amino acid alterations outside the complementarity determining regions (CDRs).

In one embodiment, the antibody comprises a variable heavy (VH) domain sequence of antibody IMPI-054, optionally with 1, 2, 3, 4 or 5 amino acid alterations outside the complementarity determining regions (CDRs), and a variable light (VL) domain sequence of antibody IMPI-054, optionally with 1, 2, 3, 4 or 5 amino acid alterations outside the complementarity determining regions (CDRs).

In one embodiment, the antibody comprises a variable heavy (VH) domain sequence of antibody IMPI-042, optionally with 1, 2, 3, 4 or 5 amino acid alterations outside the complementarity determining regions (CDRs), and a variable light (VL) domain sequence of antibody IMPI-042, optionally with 1, 2, 3, 4 or 5 amino acid alterations outside the complementarity determining regions (CDRs).

In one embodiment, the antibody comprises a variable heavy (VH) domain sequence of antibody IMPI-021, optionally with 1, 2, 3, 4 or 5 amino acid alterations outside the complementarity determining regions (CDRs), and a variable light (VL) domain sequence of antibody IMPI-021, optionally with 1, 2, 3, 4 or 5 amino acid alterations outside the complementarity determining regions (CDRs).

In one embodiment, the antibody comprises a variable heavy (VH) domain sequence of antibody IMPI-004, optionally with 1, 2, 3, 4 or 5 amino acid alterations outside the complementarity determining regions (CDRs), and a variable light (VL) domain sequence of antibody IMPI-004, optionally with 1, 2, 3, 4 or 5 amino acid alterations outside the complementarity determining regions (CDRs).

In one embodiment, the antibody comprises a variable heavy (VH) domain sequence of antibody IMPI-047, optionally with 1, 2, 3, 4 or 5 amino acid alterations outside the complementarity determining regions (CDRs), and a variable light (VL) domain sequence of antibody IMPI-047, optionally with 1, 2, 3, 4 or 5 amino acid alterations outside the complementarity determining regions (CDRs).

In one embodiment, the antibody comprises a variable heavy (VH) domain sequence of antibody IMPI-017, optionally with 1, 2, 3, 4 or 5 amino acid alterations outside the complementarity determining regions (CDRs), and a variable light (VL) domain sequence of antibody IMPI-017, optionally with 1, 2, 3, 4 or 5 amino acid alterations outside the complementarity determining regions (CDRs).

In one embodiment, the antibody comprises a variable heavy (VH) domain sequence of antibody IMPI-059, optionally with 1, 2, 3, 4 or 5 amino acid alterations outside the complementarity determining regions (CDRs), and a variable light (VL) domain sequence of antibody IMPI-059, optionally with 1, 2, 3, 4 or 5 amino acid alterations outside the complementarity determining regions (CDRs).

In one embodiment, the antibody comprises a variable heavy (VH) domain sequence of antibody IMPI-060, optionally with 1, 2, 3, 4 or 5 amino acid alterations outside the complementarity determining regions (CDRs), and a variable light (VL) domain sequence of antibody IMPI-060, optionally with 1, 2, 3, 4 or 5 amino acid alterations outside the complementarity determining regions (CDRs).

In one embodiment, the antibody comprises a variable heavy (VH) domain sequence of antibody IMPI-006, optionally with 1, 2, 3, 4 or 5 amino acid alterations outside the complementarity determining regions (CDRs), and a variable light (VL) domain sequence of antibody IMPI-006, optionally with 1, 2, 3, 4 or 5 amino acid alterations outside the complementarity determining regions (CDRs).

In one embodiment, the antibody comprises a variable heavy (VH) domain sequence of antibody IMPI-037, optionally with 1, 2, 3, 4 or 5 amino acid alterations outside the complementarity determining regions (CDRs), and a variable light (VL) domain sequence of antibody IMPI-037, optionally with 1, 2, 3, 4 or 5 amino acid alterations outside the complementarity determining regions (CDRs).

In one embodiment, the antibody comprises a variable heavy (VH) domain sequence of antibody IMPI-028, optionally with 1, 2, 3, 4 or 5 amino acid alterations outside the complementarity determining regions (CDRs), and a variable light (VL) domain sequence of antibody IMPI-028, optionally with 1, 2, 3, 4 or 5 amino acid alterations outside the complementarity determining regions (CDRs).

In one embodiment, the antibody comprises a variable heavy (VH) domain sequence and a variable light (VL) domain sequence and wherein the variable heavy (VH) domain and variable light (VL) domain sequences respectively comprise a sequence having at least 90% identity to the variable heavy (VH) and variable light (VL) domain sequences of antibody IMPI-029, IMPI-056, IMPI-005, IMPI-012, IMPI-052, IMPI-002, IMPI-041, IMPI-036, IMPI-055, IMPI-054, IMPI-042, IMPI-021, IMPI-060, IMPI-006, IMPI-004, IMPI-047, IMPI-037, IMPI-017, IMPI-059, or IMPI-028, provided that the antibody has the CDRs of antibody IMPI-029, IMPI-056, IMPI-005, IMPI-012, IMPI-052, IMPI-002, IMPI-041, IMPI-036, IMPI-055, IMPI-054, IMPI-042, IMPI-021, IMPI-060, IMPI-006, IMPI-004, IMPI-047, IMPI-037, IMPI-017, IMPI-059, or IMPI-028.

In one embodiment, the antibody comprises a variable heavy (VH) domain sequence and a variable light (VL) domain sequence and wherein the variable heavy (VH) domain and variable light (VL) domain sequences respectively comprise a sequence having at least 90% identity to the variable heavy (VH) and variable light (VL) domain sequences of antibody IMPI-029, IMPI-056, IMPI-005, IMPI-012, IMPI-052, IMPI-002, IMPI-041, IMPI-036, IMPI-055, IMPI-054, IMPI-042, IMPI-021, IMPI-004, IMPI-047, IMPI-017, IMPI-059, or IMPI-028, provided that the antibody has the CDRs of antibody IMPI-029, IMPI-056, IMPI-005, IMPI-012, IMPI-052, IMPI-002, IMPI-041, IMPI-036, IMPI-055, IMPI-054, IMPI-042, IMPI-021, IMPI-004, IMPI-047, IMPI-017, IMPI-059, or IMPI-028.

In one embodiment, the antibody binds the isolated RBD of the SARS-CoV-2 spike protein with a KD of 10-9 M or lower (e.g. as measured by surface plasmon resonance (SPR)) and the antibody comprises a variable heavy (VH) domain sequence and a variable light (VL) domain sequence and wherein the variable heavy (VH) domain and variable light (VL) domain sequences respectively comprise a sequence having at least 90% identity to the variable heavy (VH) and variable light (VL) domain sequences of antibody IMPI-004, IMPI-029, IMPI-056, IMPI-006, IMPI-055, or IMPI-059 or an antibody in the same cluster as one of these antibodies, provided that the antibody has the CDRs of antibody IMPI-004, IMPI-029, IMPI-056, IMPI-006, IMPI-055, or IMPI-059 or an antibody in the same cluster as one of these antibodies.

In one embodiment, the antibody binds the isolated RBD of the SARS-CoV-2 spike protein with a KD of 10-9 M or lower (e.g. as measured by surface plasmon resonance (SPR)) and the antibody comprises a variable heavy (VH) domain sequence and a variable light (VL) domain sequence and wherein the variable heavy (VH) domain and variable light (VL) domain sequences respectively comprise a sequence having at least 90% identity to the variable heavy (VH) and variable light (VL) domain sequences of antibody IMPI-004, IMPI-029, IMPI-056, IMPI-006, IMPI-055, or IMPI-059, provided that the antibody has the CDRs of antibody IMPI-004, IMPI-029, IMPI-056, IMPI-006, IMPI-055, or IMPI-059.

In one embodiment, the antibody binds the isolated RBD of the SARS-CoV-2 spike protein with a KD of 10-9 M or lower (e.g. as measured by surface plasmon resonance (SPR)) and the antibody comprises a variable heavy (VH) domain sequence and a variable light (VL) domain sequence and wherein the variable heavy (VH) domain and variable light (VL) domain sequences respectively comprise a sequence having at least 90% identity to the variable heavy (VH) and variable light (VL) domain sequences of antibody IMPI-004, IMPI-029, IMPI-056, IMPI-055, or IMPI-059 or an antibody in the same cluster as one of these antibodies, provided that the antibody has the CDRs of antibody IMPI-004, IMPI-029, IMPI-056, IMPI-055, or IMPI-059 or an antibody in the same cluster as one of these antibodies.

In one embodiment, the antibody binds the isolated RBD of the SARS-CoV-2 spike protein with a KD of 10-9 M or lower (e.g. as measured by surface plasmon resonance (SPR)) and the antibody comprises a variable heavy (VH) domain sequence and a variable light (VL) domain sequence and wherein the variable heavy (VH) domain and variable light (VL) domain sequences respectively comprise a sequence having at least 90% identity to the variable heavy (VH) and variable light (VL) domain sequences of antibody IMPI-004, IMPI-029, IMPI-056, IMPI-055, or IMPI-059, provided that the antibody has the CDRs of antibody IMPI-004, IMPI-029, IMPI-056, IMPI-055, or IMPI-059.

In one embodiment, the antibody neutralises SARS-CoV-2 with an IC50 of 50 pM or lower (e.g. as measured in a pseudovirus neutralisation assay) and the antibody comprises a variable heavy (VH) domain sequence and a variable light (VL) domain sequence and wherein the variable heavy (VH) domain and variable light (VL) domain sequences respectively comprise a sequence having at least 90% identity to the variable heavy (VH) and variable light (VL) domain sequences of antibody IMPI-004, IMPI-029, IMPI-055, or IMPI-059, or IMPI-017 or an antibody in the same cluster as one of these antibodies, provided that the antibody has the CDRs of antibody IMPI-004, IMPI-029, IMPI-055, or IMPI-059, or IMPI-017 or an antibody in the same cluster as one of these antibodies.

In one embodiment, the antibody neutralises SARS-CoV-2 with an IC50 of 50 pM or lower (e.g. as measured in a pseudovirus neutralisation assay) and the antibody comprises a variable heavy (VH) domain sequence and a variable light (VL) domain sequence and wherein the variable heavy (VH) domain and variable light (VL) domain sequences respectively comprise a sequence having at least 90% identity to the variable heavy (VH) and variable light (VL) domain sequences of antibody IMPI-004, IMPI-029, IMPI-055, or IMPI-059, or IMPI-017, provided that the antibody has the CDRs of antibody IMPI-004, IMPI-029, IMPI-055, or IMPI-059, or IMPI-017.

In one embodiment, the antibody binds the isolated RBD of the SARS-CoV-2 spike protein with a KD of 10-9 M or lower (e.g. as measured by surface plasmon resonance (SPR)) and the antibody neutralises SARS-CoV2 with an IC50 of 50 pM or lower (e.g. as measured in a pseudovirus neutralisation assay) and the antibody comprises a variable heavy (VH) domain sequence and a variable light (VL) domain sequence and wherein the variable heavy (VH) domain and variable light (VL) domain sequences respectively comprise a sequence having at least 90% identity to the variable heavy (VH) and variable light (VL) domain sequences of antibody IMPI-004, IMPI-029, IMPI-055, or IMPI-059 or an antibody in the same cluster as one of these antibodies, provided that the antibody has the CDRs of antibody IMPI-004, IMPI-029, IMPI-055, or IMPI-059 or an antibody in the same cluster as one of these antibodies.

In one embodiment, the antibody binds the isolated RBD of the SARS-CoV-2 spike protein with a KD of 10-9 M or lower (e.g. as measured by surface plasmon resonance (SPR)) and the antibody neutralises SARS-CoV2 with an IC50 of 50 pM or lower (e.g. as measured in a pseudovirus neutralisation assay) and the antibody comprises a variable heavy (VH) domain sequence and a variable light (VL) domain sequence and wherein the variable heavy (VH) domain and variable light (VL) domain sequences respectively comprise a sequence having at least 90% identity to the variable heavy (VH) and variable light (VL) domain sequences of antibody IMPI-004, IMPI-029, IMPI-055, or IMPI-059, provided that the antibody has the CDRs of antibody IMPI-004, IMPI-029, IMPI-055, or IMPI-059.

In one embodiment, the variable heavy (VH) domain sequence comprises a sequence having at least 90% identity to the VH domain sequence of antibody IMPI-029 and the variable light (VL) domain sequence comprises a sequence having at least 90% identity to the VL domain sequence of antibody IMPI-029, provided that the antibody has the CDRs of antibody IMPI-029.

In one embodiment, the variable heavy (VH) domain sequence comprises a sequence having at least 90% identity to the VH domain sequence of antibody IMPI-056 and the variable light (VL) domain sequence comprises a sequence having at least 90% identity to the VL domain sequence of antibody IMPI-056, provided that the antibody has the CDRs of antibody IMPI-056.

In one embodiment, the variable heavy (VH) domain sequence comprises a sequence having at least 90% identity to the VH domain sequence of antibody IMPI-005 and the variable light (VL) domain sequence comprises a sequence having at least 90% identity to the VL domain sequence of antibody IMPI-005, provided that the antibody has the CDRs of antibody IMPI-005.

In one embodiment, the variable heavy (VH) domain sequence comprises a sequence having at least 90% identity to the VH domain sequence of antibody IMPI-012 and the variable light (VL) domain sequence comprises a sequence having at least 90% identity to the VL domain sequence of antibody IMPI-012, provided that the antibody has the CDRs of antibody IMPI-012.

In one embodiment, the variable heavy (VH) domain sequence comprises a sequence having at least 90% identity to the VH domain sequence of antibody IMPI-052 and the variable light (VL) domain sequence comprises a sequence having at least 90% identity to the VL domain sequence of antibody IMPI-052, provided that the antibody has the CDRs of antibody IMPI-052.

In one embodiment, the variable heavy (VH) domain sequence comprises a sequence having at least 90% identity to the VH domain sequence of antibody IMPI-002 and the variable light (VL) domain sequence comprises a sequence having at least 90% identity to the VL domain sequence of antibody IMPI-002, provided that the antibody has the CDRs of antibody IMPI-002.

In one embodiment, the variable heavy (VH) domain sequence comprises a sequence having at least 90% identity to the VH domain sequence of antibody IMPI-041 and the variable light (VL) domain sequence comprises a sequence having at least 90% identity to the VL domain sequence of antibody IMPI-041, provided that the antibody has the CDRs of antibody IMPI-041.

In one embodiment, the variable heavy (VH) domain sequence comprises a sequence having at least 90% identity to the VH domain sequence of antibody IMPI-036 and the variable light (VL) domain sequence comprises a sequence having at least 90% identity to the VL domain sequence of antibody IMPI-036, provided that the antibody has the CDRs of antibody IMPI-036.

In one embodiment, the variable heavy (VH) domain sequence comprises a sequence having at least 90% identity to the VH domain sequence of antibody IMPI-055 and the variable light (VL) domain sequence comprises a sequence having at least 90% identity to the VL domain sequence of antibody IMPI-055, provided that the antibody has the CDRs of antibody IMPI-055.

In one embodiment, the variable heavy (VH) domain sequence comprises a sequence having at least 90% identity to the VH domain sequence of antibody IMPI-054 and the variable light (VL) domain sequence comprises a sequence having at least 90% identity to the VL domain sequence of antibody IMPI-054, provided that the antibody has the CDRs of antibody IMPI-054.

In one embodiment, the variable heavy (VH) domain sequence comprises a sequence having at least 90% identity to the VH domain sequence of antibody IMPI-042 and the variable light (VL) domain sequence comprises a sequence having at least 90% identity to the VL domain sequence of antibody IMPI-042, provided that the antibody has the CDRs of antibody IMPI-042.

In one embodiment, the variable heavy (VH) domain sequence comprises a sequence having at least 90% identity to the VH domain sequence of antibody IMPI-021 and the variable light (VL) domain sequence comprises a sequence having at least 90% identity to the VL domain sequence of antibody IMPI-021, provided that the antibody has the CDRs of antibody IMPI-021.

In one embodiment, the variable heavy (VH) domain sequence comprises a sequence having at least 90% identity to the VH domain sequence of antibody IMPI-004 and the variable light (VL) domain sequence comprises a sequence having at least 90% identity to the VL domain sequence of antibody IMPI-004, provided that the antibody has the CDRs of antibody IMPI-004.

In one embodiment, the variable heavy (VH) domain sequence comprises a sequence having at least 90% identity to the VH domain sequence of antibody IMPI-047 and the variable light (VL) domain sequence comprises a sequence having at least 90% identity to the VL domain sequence of antibody IMPI-047, provided that the antibody has the CDRs of antibody IMPI-047.

In one embodiment, the variable heavy (VH) domain sequence comprises a sequence having at least 90% identity to the VH domain sequence of antibody IMPI-017 and the variable light (VL) domain sequence comprises a sequence having at least 90% identity to the VL domain sequence of antibody IMPI-017, provided that the antibody has the CDRs of antibody IMPI-017.

In one embodiment, the variable heavy (VH) domain sequence comprises a sequence having at least 90% identity to the VH domain sequence of antibody IMPI-059 and the variable light (VL) domain sequence comprises a sequence having at least 90% identity to the VL domain sequence of antibody IMPI-059, provided that the antibody has the CDRs of antibody IMPI-059.

In one embodiment, the variable heavy (VH) domain sequence comprises a sequence having at least 90% identity to the VH domain sequence of antibody IMPI-059 and the variable light (VL) domain sequence comprises a sequence having at least 90% identity to the VL domain sequence of antibody IMPI-060, provided that the antibody has the CDRs of antibody IMPI-060.

In one embodiment, the variable heavy (VH) domain sequence comprises a sequence having at least 90% identity to the VH domain sequence of antibody IMPI-059 and the variable light (VL) domain sequence comprises a sequence having at least 90% identity to the VL domain sequence of antibody IMPI-006, provided that the antibody has the CDRs of antibody IMPI-006.

In one embodiment, the variable heavy (VH) domain sequence comprises a sequence having at least 90% identity to the VH domain sequence of antibody IMPI-059 and the variable light (VL) domain sequence comprises a sequence having at least 90% identity to the VL domain sequence of antibody IMPI-037, provided that the antibody has the CDRs of antibody IMPI-037.

In one embodiment, the variable heavy (VH) domain sequence comprises a sequence having at least 90% identity to the VH domain sequence of antibody IMPI-028 and the variable light (VL) domain sequence comprises a sequence having at least 90% identity to the VL domain sequence of antibody IMPI-028, provided that the antibody has the CDRs of antibody IMPI-028.

In one embodiment, the antibody comprises a variable heavy (VH) domain sequence and a variable light (VL) domain sequence and wherein the variable heavy (VH) domain and variable light (VL) domain sequences respectively comprise the variable heavy (VH) and variable light (VL) domain sequences of antibody IMPI-029, IMPI-056, IMPI-005, IMPI-012, IMPI-052, IMPI-002, IMPI-041, IMPI-036, IMPI-055, IMPI-054, IMPI-042, IMPI-021, IMPI-060, IMPI-006, IMPI-004, IMPI-047, IMPI-037, IMPI-017, IMPI-059, or IMPI-028.

In one embodiment, the antibody comprises a variable heavy (VH) domain sequence and a variable light (VL) domain sequence and wherein the variable heavy (VH) domain and variable light (VL) domain sequences respectively comprise the variable heavy (VH) and variable light (VL) domain sequences of antibody IMPI-029, IMPI-056, IMPI-005, IMPI-012, IMPI-052, IMPI-002, IMPI-041, IMPI-036, IMPI-055, IMPI-054, IMPI-042, IMPI-021, IMPI-004, IMPI-047, IMPI-017, IMPI-059, or IMPI-028.

In one embodiment, the present invention provides an antibody that specifically binds to the receptor binding domain (RBD) of the SARS-CoV-2 spike protein,

wherein the antibody comprises a variable heavy (VH) domain sequence and a variable light (VL) domain sequence and wherein the variable heavy (VH) domain and variable light (VL) domain sequences respectively comprise the variable heavy (VH) and variable light (VL) domain sequences of antibody IMPI-029, IMPI-056, IMPI-005, IMPI-012, IMPI-052, IMPI-002, IMPI-041, IMPI-036, IMPI-055, IMPI-054, IMPI-042, IMPI-021, IMPI-060, IMPI-006, IMPI-004, IMPI-047, IMPI-037, IMPI-017, IMPI-059, or IMPI-028.

In one embodiment, the present invention provides an antibody that specifically binds to the receptor binding domain (RBD) of the SARS-CoV-2 spike protein,

wherein the antibody comprises a variable heavy (VH) domain sequence and a variable light (VL) domain sequence and wherein the variable heavy (VH) domain and variable light (VL) domain sequences respectively comprise the variable heavy (VH) and variable light (VL) domain sequences of antibody IMPI-029, IMPI-056, IMPI-005, IMPI-012, IMPI-052, IMPI-002, IMPI-041, IMPI-036, IMPI-055, IMPI-054, IMPI-042, IMPI-021, IMPI-004, IMPI-047, IMPI-017, IMPI-059, or IMPI-028.

In one embodiment, the antibody comprises the VH and VL domain sequences of antibody IMPI-029.

In one embodiment, the antibody comprises the VH and VL domain sequences of antibody IMPI-056.

In one embodiment, the antibody comprises the VH and VL domain sequences of antibody IMPI-005.

In one embodiment, the antibody comprises the VH and VL domain sequences of antibody IMPI-012.

In one embodiment, the antibody comprises the VH and VL domain sequences of antibody IMPI-052.

In one embodiment, the antibody comprises the VH and VL domain sequences of antibody IMPI-002.

In one embodiment, the antibody comprises the VH and VL domain sequences of antibody IMPI-041.

In one embodiment, the antibody comprises the VH and VL domain sequences of antibody IMPI-036.

In one embodiment, the antibody comprises the VH and VL domain sequences of antibody IMPI-055.

In one embodiment, the antibody comprises the VH and VL domain sequences of antibody IMPI-054.

In one embodiment, the antibody comprises the VH and VL domain sequences of antibody IMPI-042.

In one embodiment, the antibody comprises the VH and VL domain sequences of antibody IMPI-021.

In one embodiment, the antibody comprises the VH and VL domain sequences of antibody IMPI-004.

In one embodiment, the antibody comprises the VH and VL domain sequences of antibody IMPI-047.

In one embodiment, the antibody comprises the VH and VL domain sequences of antibody IMPI-017.

In one embodiment, the antibody comprises the VH and VL domain sequences of antibody IMPI-059.

In one embodiment, the antibody comprises the VH and VL domain sequences of antibody IMPI-060.

In one embodiment, the antibody comprises the VH and VL domain sequences of antibody IMPI-006.

In one embodiment, the antibody comprises the VH and VL domain sequences of antibody IMPI-037.

In one embodiment, the antibody comprises the VH and VL domain sequences of antibody IMPI-028.

An antibody of the present invention may be one which competes for binding to the SARS-CoV-2 spike protein with antibody IMPI-037.

In one embodiment, an antibody is provided which binds to the same epitope as antibody IMPI-037.

In one embodiment, the antibody comprises VH and/or VL domain framework regions of human germline gene segment sequences.

In one embodiment, the antibody comprises an antibody VH domain which

i) is derived from recombination of a human heavy chain V gene segment, a human heavy chain D gene segment and a human heavy chain J gene segment, wherein
the V gene segment is IGHV3-53*01, IGHV1-8*01 or IGHV3-33*01;
and/or
the J gene segment is IGHJ6*02, IGHJ4*02 or IGHJ3*02, or
   ii) comprises framework regions FR1, FR2, FR3 and FR4, wherein
FR1 aligns with human germline V gene segment IGHV3-53*01, IGHV1-8*01 or IGHV3-33*01 with up to 1, 2, 3, 4, or 5 amino acid alterations,
FR2 aligns with human germline V gene segment IGHV3-53*01, IGHV1-8*01 or IGHV3-33*01 with up to 1, 2, 3, 4, or 5 amino acid alterations,
FR3 aligns with human germline V gene segment IGHV3-53*01, IGHV1-8*01 or IGHV3-33*01 with up to 1, 2, 3, 4 or 5 amino acid alterations, and/or
FR4 aligns with human germline J gene segment IGHJ6*02, IGHJ4*02 or IGHJ3*02 with up to 1, 2, 3, 4 or 5 amino acid alterations.

In one embodiment, the antibody comprises an antibody VH domain which
i) is derived from recombination of a human heavy chain V gene segment IGHV3-53*01, IGHV1-8*01 or IGHV3-33*01, a human heavy chain D gene segment and a human heavy chain J gene segment, or
ii) comprises framework regions FR1, FR2, FR3 and FR4, wherein FR1, FR2 and FR3 each align with human germline V segment IGHV3-53*01, IGHV1-8*01 or IGHV3-33*01 with up to 1, 2, 3, 4 or 5 amino acid alterations.

In one embodiment, the J gene segment is IGHJ6*02, IGHJ4*02 or IGHJ3*02, or the VH domain framework region FR4 aligns with human germline J gene segment IGHJ6*02, IGHJ4*02 or IGHJ3*02 with 1, 2, 3, 4 or 5 amino acid alterations.

In one embodiment, the antibody comprises an antibody VL domain which
i) is derived from recombination of a human light chain V gene segment and a human light chain J gene segment, wherein the V gene segment is IGKV1-9*d01, IGKV6-21*01, IGKV1-33*01 or IGKV3-20*01, and/or the J gene segment is IGKJ5*01, IGKJ4*01, IGKJ3*01, IGKJ2*04 or IGKJ1*01; or
ii) comprises framework regions FR1, FR2, FR3 and FR4, wherein FR1 aligns with human germline V gene segment IGKV1-9*d01, IGKV6-21*01, IGKV1-33*01 or IGKV3-20*01 with up to 1, 2, 3, 4, or 5 amino acid alterations, FR2 aligns with human germline V gene segment IGKV1-9*d01, IGKV6-21*01, IGKV1-33*01 or IGKV3-20*01 with up to 1, 2, 3, 4, or 5 amino acid alterations FR3 aligns with human germline V gene segment IGKV1-9*d01, IGKV6-21*01, IGKV1-33*01 or IGKV3-20*01 with up to 1, 2, 3, 4 or 5 amino acid alterations, and/or FR4 aligns with human germline J gene segment IGKJ5*01, IGKJ4*01, IGKJ3*01, IGKJ2*04 or IGKJ1*01 with up to 1, 2, 3, 4 or 5 amino acid alterations.

In one embodiment, the antibody comprises an antibody VL domain derived from recombination of a human light chain V gene segment and a human light chain J gene segment, wherein: the V gene segment is IGKV1-9*d01, IGKV6-21*01, IGKV1-33*01 or IGKV3-20*01, and optionally the J gene segment is IGKJ5*01, IGKJ4*01, IGKJ3*01, IGKJ2*04 or IGKJ1*01.

Example combinations of v and j gene segments for heavy and light chain variable domains are shown in Table 3, and these represent preferred combinations. The heavy and light chain variable domains may optionally be derived from the v and j gene segments identified in Table 3 for any one individual IMPI antibody identified in this Group A section.

### Further Competing RBD Binders

According to the first aspect of the invention, further antibodies are provided herein which specifically bind to the RBD of the SARS-CoV-2 spike protein and compete for binding to the SARS-CoV-2 spike protein with the human ACE2 receptor. For example, antibodies YANG-1101, YANG-1103, YANG-1105, YANG-1106, YANG-1107, YANG-1108, YANG-1109, YANG-1110, YANG-1112, YANG-1112a, YANG-1112b, YANG-1112c, YANG-1113, YANG-1114, YANG-1115, YANG-1116, YANG-1117, YANG-1118, YANG-1119, YANG-2101, YANG-2102, YANG-2103, YANG-2104, YANG-2105, YANG-2106, YANG-2107, YANG-2108, YANG-2108a, YANG-2108b, YANG-2108c, YANG-2108d, YANG-2108e, YANG-2108f, YANG-2108g, YANG-2108h, YANG-2108i, YANG-2108j, YANG-2108k, YANG-21081, YANG-2109, YANG-2110, and YANG-2111 as exemplified herein have been found to specifically bind to the RBD of the SARS-CoV-2 spike protein and to compete for binding to the SARS-CoV-2 spike protein with the human ACE2 receptor.

In one embodiment, the antibody comprises a variable heavy (VH) domain sequence comprising complementarity determining regions (CDRs) HCDR1, HCDR2 and HCDR3, and a variable light (VL) domain sequence comprising complementarity determining regions LCDR1, LCDR2 and LCDR3, wherein the HCDR3 is the HCDR3 of antibody YANG-1101, YANG-1103, YANG-1105, YANG-1106, YANG-1107, YANG-1108, YANG-1109, YANG-1110, YANG-1112, YANG-1112a, YANG-1112b, YANG-1112c, YANG-1113, YANG-1114, YANG-1115, YANG-1116, YANG-1117, YANG-1118, YANG-1119, YANG-2101, YANG-2102, YANG-2103, YANG-2104, YANG-2105, YANG-2106, YANG-2107, YANG-2108, YANG-2108a, YANG-2108b, YANG-2108c, YANG-2108d, YANG-2108e, YANG-2108f, YANG-2108g, YANG-2108h, YANG-2108i, YANG-2108j, YANG-2108k, YANG-21081, YANG-2109, YANG-2110, or YANG-2111.

In one embodiment, the antibody comprises a variable heavy (VH) domain sequence comprising complementarity determining regions (CDRs) HCDR1, HCDR2 and HCDR3, and a variable light (VL) domain sequence comprising complementarity determining regions LCDR1, LCDR2 and LCDR3, wherein HCDR3 is the HCDR3 of antibody YANG-1112, YANG-2107, YANG-2108, or YANG-2111.

In one embodiment, HCDR3 is the HCDR3 of antibody YANG-1101.

In one embodiment, HCDR3 is the HCDR3 of antibody YANG-1103.

In one embodiment, HCDR3 is the HCDR3 of antibody YANG-1105.

In one embodiment, HCDR3 is the HCDR3 of antibody YANG-1106.

In one embodiment, HCDR3 is the HCDR3 of antibody YANG-1107.

In one embodiment, HCDR3 is the HCDR3 of antibody YANG-1108.

In one embodiment, HCDR3 is the HCDR3 of antibody YANG-1109.

In one embodiment, HCDR3 is the HCDR3 of antibody YANG-1110.

In one embodiment, HCDR3 is the HCDR3 of antibody YANG-1112.

In one embodiment, HCDR3 is the HCDR3 of antibody YANG-1113.

In one embodiment, HCDR3 is the HCDR3 of antibody YANG-1114.

In one embodiment, HCDR3 is the HCDR3 of antibody YANG-1115.

In one embodiment, HCDR3 is the HCDR3 of antibody YANG-1116.

In one embodiment, HCDR3 is the HCDR3 of antibody YANG-1117.

In one embodiment, HCDR3 is the HCDR3 of antibody YANG-1118.

In one embodiment, HCDR3 is the HCDR3 of antibody YANG-1119.

In one embodiment, HCDR3 is the HCDR3 of antibody YANG-2101.

In one embodiment, HCDR3 is the HCDR3 of antibody YANG-2102.

In one embodiment, HCDR3 is the HCDR3 of antibody YANG-2103.

In one embodiment, HCDR3 is the HCDR3 of antibody YANG-2104.

In one embodiment, HCDR3 is the HCDR3 of antibody YANG-2105.

In one embodiment, HCDR3 is the HCDR3 of antibody YANG-2106.

In one embodiment, HCDR3 is the HCDR3 of antibody YANG-2107.

In one embodiment, HCDR3 is the HCDR3 of antibody YANG-2108.

In one embodiment, HCDR3 is the HCDR3 of antibody YANG-2109.

In one embodiment, HCDR3 is the HCDR3 of antibody YANG-2110.

In one embodiment, HCDR3 is the HCDR3 of antibody YANG-2111.

In one embodiment, the antibody specifically binds to the RBD of the SARS-CoV-2 spike protein and competes for binding to the SARS-CoV-2 spike protein with the human ACE2 receptor, and HCDR3 is the HCDR3 of antibody YANG-1101, YANG-1103, YANG-1105, YANG-1106, YANG-1107, YANG-1108, YANG-1109, YANG-1110, YANG-1112, YANG-1112a, YANG-1112b, YANG-1112c, YANG-1113, YANG-1114, YANG-1115, YANG-1116, YANG-1117, YANG-1118, YANG-1119, YANG-2101, YANG-2102, YANG-2103, YANG-2104, YANG-2105, YANG-2106, YANG-2107, YANG-2108, YANG-2108a, YANG-2108b, YANG-2108c, YANG-2108d, YANG-2108e, YANG-2108f, YANG-2108g, YANG-2108h, YANG-2108i, YANG-2108j, YANG-2108k, YANG-21081, YANG-2109, YANG-2110, or YANG-2111.

In one embodiment, the antibody specifically binds to the RBD of the SARS-CoV-2 spike protein with a KD of 0.1 nM or lower (e.g. as measured by surface plasmon resonance (SPR)) and competes for binding to the SARS-CoV-2 spike protein with the human ACE2 receptor, and HCDR3 is the HCDR3 of antibody YANG-1101, YANG-1103, YANG-1105, YANG-1106, YANG-1107, YANG-1108, YANG-1109, YANG-1110, YANG-1112, YANG-1112a, YANG-1112b, YANG-1112c, YANG-1113, YANG-1114, YANG-1115, YANG-1116, YANG-1117, YANG-1118, YANG-1119, YANG-2101, YANG-2102, YANG-2103, YANG-2104, YANG-2105, YANG-2106, YANG-2107, YANG-2108, YANG-2108a, YANG-2108b, YANG-2108c, YANG-2108d, YANG-2108e, YANG-2108f, YANG-2108g, YANG-2108h, YANG-2108i, YANG-2108j, YANG-2108k, YANG-21081, YANG-2109, YANG-2110, or YANG-2111.

In one embodiment, the antibody binds the isolated RBD of the SARS-CoV-2 spike protein with a KD of 0.1 nM or lower (e.g. as measured by surface plasmon resonance (SPR)) and HCDR3 is the HCDR3 of antibody YANG-1112, YANG-2107, YANG-2108, or YANG-2111.

In one embodiment, the antibody specifically binds to the S2 subunit of the SARS-CoV-2 spike protein and neutralises SARS-CoV-2 with at least 60% neutralisation (e.g. as measured in a pseudovirus neutralisation assay), and HCDR3 is the HCDR3 of antibody YANG-1112, YANG-2107, YANG-2108, or YANG-2111.

In one embodiment, the antibody specifically binds to the S2 subunit of the SARS-CoV-2 spike protein with a KD of 0.1 nM or lower (e.g. as measured by surface plasmon resonance (SPR)) and neutralises SARS-CoV-2 with at least 60% neutralisation (e.g. as measured in a pseudovirus neutralisation assay), and HCDR3 is the HCDR3 of antibody YANG-1112, YANG-2107, YANG-2108, or YANG-2111.

In one embodiment, the present invention provides an anti-SARS-CoV-2 antibody, wherein the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3, wherein the CDRs are those of antibody YANG-1101, YANG-1103, YANG-1105, YANG-1106, YANG-1107, YANG-1108, YANG-1109, YANG-1110, YANG-1112, YANG-1112a, YANG-1112b, YANG-1112c, YANG-1113, YANG-1114, YANG-1115, YANG-1116, YANG-1117, YANG-1118, YANG-1119, YANG-2101, YANG-2102, YANG-2103, YANG-2104, YANG-2105, YANG-2106, YANG-2107, YANG-2108, YANG-2108a, YANG-2108b, YANG-2108c, YANG-2108d, YANG-2108e, YANG-2108f, YANG-2108g, YANG-2108h, YANG-2108i, YANG-2108j, YANG-2108k, YANG-21081, YANG-2109, YANG-2110, or YANG-2111.

In one embodiment, the present invention provides an anti-SARS-CoV-2 antibody, wherein the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3, wherein the CDRs are those of antibody YANG-1112, YANG-2107, YANG-2108, or YANG-2111.

In one embodiment, the antibody has the CDRs of antibody YANG-1101.

In one embodiment, the antibody has the CDRs of antibody YANG-1103.

In one embodiment, the antibody has the CDRs of antibody YANG-1105.

In one embodiment, the antibody has the CDRs of antibody YANG-1106.

In one embodiment, the antibody has the CDRs of antibody YANG-1107.

In one embodiment, the antibody has the CDRs of antibody YANG-1108.

In one embodiment, the antibody has the CDRs of antibody YANG-1109.

In one embodiment, the antibody has the CDRs of antibody YANG-1110.

In one embodiment, the antibody has the CDRs of antibody YANG-1112.

In one embodiment, the antibody has the CDRs of antibody YANG-1113.

In one embodiment, the antibody has the CDRs of antibody YANG-1114.

In one embodiment, the antibody has the CDRs of antibody YANG-1115.

In one embodiment, the antibody has the CDRs of antibody YANG-1116.

In one embodiment, the antibody has the CDRs of antibody YANG-1117.

In one embodiment, the antibody has the CDRs of antibody YANG-1118.

In one embodiment, the antibody has the CDRs of antibody YANG-1119.

In one embodiment, the antibody has the CDRs of antibody YANG-2101.

In one embodiment, the antibody has the CDRs of antibody YANG-2102.

In one embodiment, the antibody has the CDRs of antibody YANG-2103.

In one embodiment, the antibody has the CDRs of antibody YANG-2104.

In one embodiment, the antibody has the CDRs of antibody YANG-2105.

In one embodiment, the antibody has the CDRs of antibody YANG-2106.

In one embodiment, the antibody has the CDRs of antibody YANG-2107.

In one embodiment, the antibody has the CDRs of antibody YANG-2108.

In one embodiment, the antibody has the CDRs of antibody YANG-2109.

In one embodiment, the antibody has the CDRs of antibody YANG-2110.

In one embodiment, the antibody has the CDRs of antibody YANG-2111.

In one embodiment, the antibody specifically binds to the RBD of the SARS-CoV-2 spike protein and competes for binding to the SARS-CoV-2 spike protein with the human ACE2 receptor, and the CDRs are those of antibody YANG-1101, YANG-1103, YANG-1105, YANG-1106, YANG-1107, YANG-1108, YANG-1109, YANG-1110, YANG-1112, YANG-1112a, YANG-1112b, YANG-1112c, YANG-1113, YANG-1114, YANG-1115, YANG-1116, YANG-1117, YANG-1118, YANG-1119, YANG-2101, YANG-2102, YANG-2103, YANG-2104, YANG-2105, YANG-2106, YANG-2107, YANG-2108, YANG-2108a, YANG-2108b, YANG-2108c, YANG-2108d, YANG-2108e, YANG-2108f, YANG-2108g, YANG-2108h, YANG-2108i, YANG-2108j, YANG-2108k, YANG-21081, YANG-2109, YANG-2110, or YANG-2111.

In one embodiment, the antibody specifically binds to the RBD of the SARS-CoV-2 spike protein with a KD of 0.1 nM or lower (e.g. as measured by surface plasmon resonance (SPR)) and competes for binding to the SARS-CoV-2 spike protein with the human ACE2 receptor, and the CDRs are those of antibody YANG-1101, YANG-1103, YANG-1105, YANG-1106, YANG-1107, YANG-1108, YANG-1109, YANG-1110, YANG-1112, YANG-1112a, YANG-1112b, YANG-1112c, YANG-1113, YANG-1114, YANG-1115, YANG-1116, YANG-1117, YANG-1118, YANG-1119, YANG-2101, YANG-2102, YANG-2103, YANG-2104, YANG-2105, YANG-2106, YANG-2107, YANG-2108, YANG-2108a, YANG-2108b, YANG-2108c, YANG-2108d, YANG-2108e, YANG-2108f, YANG-2108g, YANG-2108h, YANG-2108i, YANG-2108j, YANG-2108k, YANG-21081, YANG-2109, YANG-2110, or YANG-2111.

In one embodiment, the antibody specifically binds to the RBD of the SARS-CoV-2 spike protein and competes for binding to the SARS-CoV-2 spike protein with the human ACE2 receptor, and the CDRs are those of antibody YANG-1112, YANG-2107, YANG-2108, or YANG-2111.

In one embodiment, the antibody specifically binds to the RBD of the SARS-CoV-2 spike protein with a KD of 0.1 nM or lower (e.g. as measured by surface plasmon resonance (SPR)) and competes for binding to the SARS-CoV-2 spike protein with the human ACE2 receptor, and the CDRs are those of antibody YANG-1112, YANG-2107, YANG-2108, or YANG-2111.

In one embodiment, the antibody specifically binds to the S2 subunit of the SARS-CoV-2 spike protein and competes for binding to the SARS-CoV-2 spike protein with the human ACE2 receptor and neutralises SARS-CoV-2 with at least 60% neutralisation (e.g. as measured in a pseudovirus neutralisation assay), and the CDRs are those of antibody YANG-1112, YANG-2107, YANG-2108, or YANG-2111.

In one embodiment, the antibody specifically binds to the S2 subunit of the SARS-CoV-2 spike protein with a KD of 0.1 nM or lower (e.g. as measured by surface plasmon resonance (SPR)) and competes for binding to the SARS-CoV-2 spike protein with the human ACE2 receptor and neutralises SARS-CoV-2 with at least 60% neutralisation (e.g. as measured in a pseudovirus neutralisation assay), and the CDRs are those of antibody YANG-1112, YANG-2107, YANG-2108, or YANG-2111.

In one embodiment, the antibody comprises a variable heavy (VH) domain sequence and a variable light (VL) domain sequence and wherein the variable heavy (VH) domain and variable light (VL) domain sequences respectively comprise the variable heavy (VH) domain and variable light (VL) domain sequences of antibody YANG-1101, YANG-1103, YANG-1105, YANG-1106, YANG-1107, YANG-1108, YANG-1109, YANG-1110, YANG-1112, YANG-1112a, YANG-1112b, YANG-1112c, YANG-1113, YANG-1114, YANG-1115, YANG-1116, YANG-1117, YANG-1118, YANG-1119, YANG-2101, YANG-2102, YANG-2103, YANG-2104, YANG-2105, YANG-2106, YANG-2107, YANG-2108, YANG-2108a, YANG-2108b, YANG-2108c, YANG-2108d, YANG-2108e, YANG-2108f, YANG-2108g, YANG-2108h, YANG-2108i, YANG-2108j, YANG-2108k, YANG-21081, YANG-2109, YANG-2110, or YANG-2111, YANG-2111a, YANG-2111b, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

In one embodiment, the antibody comprises a variable heavy (VH) domain sequence and a variable light (VL) domain sequence and wherein the variable heavy (VH) domain and variable light (VL) domain sequences respectively comprise the variable heavy (VH) domain and variable light (VL) domain sequences of antibody YANG-1112, YANG-2107, YANG-2108, or YANG-2111 optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

In one embodiment, the antibody comprises a variable heavy (VH) domain sequence of antibody YANG-1101, optionally with 1, 2, 3, 4 or 5 amino acid alterations outside the complementarity determining regions (CDRs), and a variable light (VL) domain sequence of antibody YANG-1101 optionally with 1, 2, 3, 4 or 5 amino acid alterations outside the complementarity determining regions (CDRs).

In one embodiment, the antibody comprises a variable heavy (VH) domain sequence of antibody YANG-1103, optionally with 1, 2, 3, 4 or 5 amino acid alterations outside the complementarity determining regions (CDRs), and a variable light (VL) domain sequence of antibody YANG-1103, optionally with 1, 2, 3, 4 or 5 amino acid alterations outside the complementarity determining regions (CDRs).

In one embodiment, the antibody comprises a variable heavy (VH) domain sequence of antibody YANG-1105, optionally with 1, 2, 3, 4 or 5 amino acid alterations outside the complementarity determining regions (CDRs), and a variable light (VL) domain sequence of antibody YANG-1105, optionally with 1, 2, 3, 4 or 5 amino acid alterations outside the complementarity determining regions (CDRs).

In one embodiment, the antibody comprises a variable heavy (VH) domain sequence of antibody YANG-1106, optionally with 1, 2, 3, 4 or 5 amino acid alterations outside the complementarity determining regions (CDRs), and a variable light (VL) domain sequence of antibody YANG-1106, optionally with 1, 2, 3, 4 or 5 amino acid alterations outside the complementarity determining regions (CDRs).

In one embodiment, the antibody comprises a variable heavy (VH) domain sequence of antibody YANG-1107, optionally with 1, 2, 3, 4 or 5 amino acid alterations outside the complementarity determining regions (CDRs), and a variable light (VL) domain sequence of antibody YANG-1107, optionally with 1, 2, 3, 4 or 5 amino acid alterations outside the complementarity determining regions (CDRs).

In one embodiment, the antibody comprises a variable heavy (VH) domain sequence of antibody YANG-1108, optionally with 1, 2, 3, 4 or 5 amino acid alterations outside the complementarity determining regions (CDRs), and a variable light (VL) domain sequence of antibody YANG-1108, optionally with 1, 2, 3, 4 or 5 amino acid alterations outside the complementarity determining regions (CDRs).

In one embodiment, the antibody comprises a variable heavy (VH) domain sequence of antibody YANG-1109, optionally with 1, 2, 3, 4 or 5 amino acid alterations outside the complementarity determining regions (CDRs), and a variable light (VL) domain sequence of antibody YANG-1109, optionally with 1, 2, 3, 4 or 5 amino acid alterations outside the complementarity determining regions (CDRs).

In one embodiment, the antibody comprises a variable heavy (VH) domain sequence of antibody YANG-1110 optionally with 1, 2, 3, 4 or 5 amino acid alterations outside the complementarity determining regions (CDRs), and a variable light (VL) domain sequence of antibody YANG-1110, optionally with 1, 2, 3, 4 or 5 amino acid alterations outside the complementarity determining regions (CDRs).

In one embodiment, the antibody comprises a variable heavy (VH) domain sequence of antibody YANG-1112 optionally with 1, 2, 3, 4 or 5 amino acid alterations outside the complementarity determining regions (CDRs), and a variable light (VL) domain sequence of antibody YANG-1112, optionally with 1, 2, 3, 4 or 5 amino acid alterations outside the complementarity determining regions (CDRs).

In one embodiment, the antibody comprises a variable heavy (VH) domain sequence of antibody YANG-1113 optionally with 1, 2, 3, 4 or 5 amino acid alterations outside the complementarity determining regions (CDRs), and a variable light (VL) domain sequence of antibody YANG-1113, optionally with 1, 2, 3, 4 or 5 amino acid alterations outside the complementarity determining regions (CDRs).

In one embodiment, the antibody comprises a variable heavy (VH) domain sequence of antibody YANG-1114 optionally with 1, 2, 3, 4 or 5 amino acid alterations outside the complementarity determining regions (CDRs), and a variable light (VL) domain sequence of antibody YANG-1114, optionally with 1, 2, 3, 4 or 5 amino acid alterations outside the complementarity determining regions (CDRs).

In one embodiment, the antibody comprises a variable heavy (VH) domain sequence of antibody YANG-1115 optionally with 1, 2, 3, 4 or 5 amino acid alterations outside the complementarity determining regions (CDRs), and a variable light (VL) domain sequence of antibody YANG-1115, optionally with 1, 2, 3, 4 or 5 amino acid alterations outside the complementarity determining regions (CDRs).

In one embodiment, the antibody comprises a variable heavy (VH) domain sequence of antibody YANG-1116 optionally with 1, 2, 3, 4 or 5 amino acid alterations outside the complementarity determining regions (CDRs), and a variable light (VL) domain sequence of antibody YANG-1116, optionally with 1, 2, 3, 4 or 5 amino acid alterations outside the complementarity determining regions (CDRs).

In one embodiment, the antibody comprises a variable heavy (VH) domain sequence of antibody YANG-1117 optionally with 1, 2, 3, 4 or 5 amino acid alterations outside the complementarity determining regions (CDRs), and a variable light (VL) domain sequence of antibody YANG-1117, optionally with 1, 2, 3, 4 or 5 amino acid alterations outside the complementarity determining regions (CDRs).

In one embodiment, the antibody comprises a variable heavy (VH) domain sequence of antibody YANG-1118 optionally with 1, 2, 3, 4 or 5 amino acid alterations outside the complementarity determining regions (CDRs), and a variable light (VL) domain sequence of antibody YANG-1118, optionally with 1, 2, 3, 4 or 5 amino acid alterations outside the complementarity determining regions (CDRs).

In one embodiment, the antibody comprises a variable heavy (VH) domain sequence of antibody YANG-1119 optionally with 1, 2, 3, 4 or 5 amino acid alterations outside the complementarity determining regions (CDRs), and a variable light (VL) domain sequence of antibody YANG-1119, optionally with 1, 2, 3, 4 or 5 amino acid alterations outside the complementarity determining regions (CDRs).

In one embodiment, the antibody comprises a variable heavy (VH) domain sequence of antibody YANG-2101, optionally with 1, 2, 3, 4 or 5 amino acid alterations outside the complementarity determining regions (CDRs), and a variable light (VL) domain sequence of antibody YANG-2101, optionally with 1, 2, 3, 4 or 5 amino acid alterations outside the complementarity determining regions (CDRs).

In one embodiment, the antibody comprises a variable heavy (VH) domain sequence of antibody YANG-2102, optionally with 1, 2, 3, 4 or 5 amino acid alterations outside the complementarity determining regions (CDRs), and a variable light (VL) domain sequence of antibody YANG-2102, optionally with 1, 2, 3, 4 or 5 amino acid alterations outside the complementarity determining regions (CDRs).

In one embodiment, the antibody comprises a variable heavy (VH) domain sequence of antibody YANG-2103, optionally with 1, 2, 3, 4 or 5 amino acid alterations outside the complementarity determining regions (CDRs), and a variable light (VL) domain sequence of antibody YANG-2103, optionally with 1, 2, 3, 4 or 5 amino acid alterations outside the complementarity determining regions (CDRs).

In one embodiment, the antibody comprises a variable heavy (VH) domain sequence of antibody YANG-2104, optionally with 1, 2, 3, 4 or 5 amino acid alterations outside the complementarity determining regions (CDRs), and a variable light (VL) domain sequence of antibody YANG-2104, optionally with 1, 2, 3, 4 or 5 amino acid alterations outside the complementarity determining regions (CDRs).

In one embodiment, the antibody comprises a variable heavy (VH) domain sequence of antibody YANG-2105, optionally with 1, 2, 3, 4 or 5 amino acid alterations outside the complementarity determining regions (CDRs), and a variable light (VL) domain sequence of antibody YANG-2105, optionally with 1, 2, 3, 4 or 5 amino acid alterations outside the complementarity determining regions (CDRs).

In one embodiment, the antibody comprises a variable heavy (VH) domain sequence of antibody YANG-2106, optionally with 1, 2, 3, 4 or 5 amino acid alterations outside the complementarity determining regions (CDRs), and a variable light (VL) domain sequence of antibody YANG-2106, optionally with 1, 2, 3, 4 or 5 amino acid alterations outside the complementarity determining regions (CDRs).

In one embodiment, the antibody comprises a variable heavy (VH) domain sequence of antibody YANG-2107, optionally with 1, 2, 3, 4 or 5 amino acid alterations outside the complementarity determining regions (CDRs), and a variable light (VL) domain sequence of antibody YANG-2107, optionally with 1, 2, 3, 4 or 5 amino acid alterations outside the complementarity determining regions (CDRs).

In one embodiment, the antibody comprises a variable heavy (VH) domain sequence of antibody YANG-2108, optionally with 1, 2, 3, 4 or 5 amino acid alterations outside the complementarity determining regions (CDRs), and a variable light (VL) domain sequence of antibody YANG-2108, optionally with 1, 2, 3, 4 or 5 amino acid alterations outside the complementarity determining regions (CDRs).

In one embodiment, the antibody comprises a variable heavy (VH) domain sequence of antibody YANG-2109, optionally with 1, 2, 3, 4 or 5 amino acid alterations outside the complementarity determining regions (CDRs), and a variable light (VL) domain sequence of antibody YANG-2109, optionally with 1, 2, 3, 4 or 5 amino acid alterations outside the complementarity determining regions (CDRs).

In one embodiment, the antibody comprises a variable heavy (VH) domain sequence of antibody YANG-2110, optionally with 1, 2, 3, 4 or 5 amino acid alterations outside the complementarity determining regions (CDRs), and a variable light (VL) domain sequence of antibody YANG-2110, optionally with 1, 2, 3, 4 or 5 amino acid alterations outside the complementarity determining regions (CDRs).

In one embodiment, the antibody comprises a variable heavy (VH) domain sequence of antibody YANG-2111, optionally with 1, 2, 3, 4 or 5 amino acid alterations outside the complementarity determining regions (CDRs), and a variable light (VL) domain sequence of antibody YANG-2111, optionally with 1, 2, 3, 4 or 5 amino acid alterations outside the complementarity determining regions (CDRs).

In one embodiment, the antibody specifically binds to the RBD of the SARS-CoV-2 spike protein and competes for binding to the SARS-CoV-2 spike protein with the human ACE2 receptor, and the variable heavy (VH) domain and variable light (VL) domain sequences respectively comprise the variable heavy (VH) domain and variable light (VL) domain sequences of antibody YANG-1101, YANG-1103, YANG-1105, YANG-1106, YANG-1107, YANG-1108, YANG-1109, YANG-1110, YANG-1112, YANG-1112a, YANG-1112b, YANG-1112c, YANG-1113, YANG-1114, YANG-1115, YANG-1116, YANG-1117, YANG-1118, YANG-1119, YANG-2101, YANG-2102, YANG-2103, YANG-2104, YANG-2105, YANG-2106, YANG-2107, YANG-2108, YANG-2108a, YANG-2108b, YANG-2108c, YANG-2108d, YANG-2108e, YANG-2108f, YANG-2108g, YANG-2108h, YANG-2108i, YANG-2108j, YANG-2108k, YANG-21081, YANG-2109, YANG-2110, orYANG-2111, YANG-2111a, YANG-2111b, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

In one embodiment, the antibody specifically binds to the RBD of the SARS-CoV-2 spike protein with a KD of 0.1 nM or lower (e.g. as measured by surface plasmon resonance (SPR)) and competes for binding to the SARS-CoV-2 spike protein with the human ACE2 receptor, and the variable heavy (VH) domain and variable light (VL) domain sequences respectively comprise the variable heavy (VH) domain and variable light (VL) domain sequences of antibody YANG-1101, YANG-1103, YANG-1105, YANG-1106, YANG-1107, YANG-1108, YANG-1109, YANG-1110, YANG-1112, YANG-1112a, YANG-1112b, YANG-1112c, YANG-1113, YANG-1114, YANG-1115, YANG-1116, YANG-1117, YANG-1118, YANG-1119, YANG-2101, YANG-2102, YANG-2103, YANG-2104, YANG-2105, YANG-2106, YANG-2107, YANG-2108, YANG-2108a, YANG-2108b, YANG-2108c, YANG-2108d, YANG-2108e, YANG-2108f, YANG-2108g, YANG-2108h, YANG-2108i, YANG-2108j, YANG-2108k, YANG-21081, YANG-2109, YANG-2110, orYANG-2111, YANG-2111a, YANG-2111, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

In one embodiment, the antibody specifically binds to the RBD of the SARS-CoV-2 spike protein and competes for binding to the SARS-CoV-2 spike protein with the human ACE2 receptor, and the variable heavy (VH) domain and variable light (VL) domain sequences respectively comprise the variable heavy (VH) domain and variable light (VL) domain sequences of antibody YANG-1112, YANG-2107, YANG-2108, or YANG-2111, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

In one embodiment, the antibody specifically binds to the RBD of the SARS-CoV-2 spike protein with a KD of 0.1 nM or lower (e.g. as measured by surface plasmon resonance (SPR)) and competes for binding to the SARS-CoV-2 spike protein with the human ACE2 receptor, and the variable heavy (VH) domain and variable light (VL) domain sequences respectively comprise the variable heavy (VH) domain and variable light (VL) domain sequences of antibody YANG-1112, YANG-2107, YANG-2108, or YANG-2111, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

In one embodiment, the antibody specifically binds to the S2 subunit of the SARS-CoV-2 spike protein and competes for binding to the SARS-CoV-2 spike protein with the human ACE2 receptor and neutralises SARS-CoV-2 with at least 60% neutralisation (e.g. as measured in a pseudovirus neutralisation assay), and the variable heavy (VH) domain and variable light (VL) domain sequences respectively comprise the variable heavy (VH) domain and variable light (VL) domain sequences of antibody YANG-1112, YANG-2107, YANG-2108, or YANG-2111, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

In one embodiment, the antibody specifically binds to the S2 subunit of the SARS-CoV-2 spike protein with a KD of 0.1 nM or lower (e.g. as measured by surface plasmon resonance (SPR)) and competes for binding to the SARS-CoV-2 spike protein with the human ACE2 receptor and neutralises SARS-CoV-2 with at least 60% neutralisation (e.g. as measured in a pseudovirus neutralisation assay), and the variable heavy (VH) domain and variable light (VL) domain sequences respectively comprise the variable heavy (VH) domain and variable light (VL) domain sequences of antibody YANG-1112, YANG-2107, YANG-2108, or YANG-2111, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

In one embodiment, the antibody comprises a variable heavy (VH) domain sequence and a variable light (VL) domain sequence and wherein the variable heavy (VH) domain and variable light (VL) domain sequences respectively comprise a sequence having at least 90% identity to the variable heavy (VH) and variable light (VL) domain sequences of antibody YANG-1101, YANG-1103, YANG-1105, YANG-1106, YANG-1107, YANG-1108, YANG-1109, YANG-1110, YANG-1112, YANG-1112a, YANG-1112b, YANG-1112c, YANG-1113, YANG-1114, YANG-1115, YANG-1116, YANG-1117, YANG-1118, YANG-1119, YANG-2101, YANG-2102, YANG-2103, YANG-2104, YANG-2105, YANG-2106, YANG-2107, YANG-2108, YANG-2108a, YANG-2108b, YANG-2108c, YANG-2108d, YANG-2108e, YANG-2108f, YANG-2108g, YANG-2108h, YANG-2108i, YANG-2108j, YANG-2108k, YANG-21081, YANG-2109, YANG-2110, or YANG-2111 provided that the antibody has the CDRs of antibody YANG-1101, YANG-1103, YANG-1105, YANG-1106, YANG-1107, YANG-1108, YANG-1109, YANG-1110, YANG-1112, YANG-1112a, YANG-1112b, YANG-1112c, YANG-1113, YANG-1114, YANG-1115, YANG-1116, YANG-1117, YANG-1118, YANG-1119, YANG-2101, YANG-2102, YANG-2103, YANG-2104, YANG-2105, YANG-2106, YANG-2107, YANG-2108, YANG-2108a, YANG-2108b, YANG-2108c, YANG-2108d, YANG-2108e, YANG-2108f, YANG-2108g, YANG-2108h, YANG-2108i, YANG-2108j, YANG-2108k, YANG-21081, YANG-2109, YANG-2110, or YANG-2111, YANG-2111a, YANG-2111b, respectively.

In one embodiment, the antibody comprises a variable heavy (VH) domain sequence and a variable light (VL) domain sequence and wherein the variable heavy (VH) domain and variable light (VL) domain sequences respectively comprise a sequence having at least 90% identity to the variable heavy (VH) and variable light (VL) domain sequences of antibody YANG-1112, YANG-2107, YANG-2108, or YANG-2111, provided that the antibody has the CDRs of antibody YANG-1112, YANG-2107, YANG-2108, or YANG-2111, respectively.

In one embodiment, the variable heavy (VH) domain sequence comprises a sequence having at least 90% identity to the VH domain sequence of antibody YANG-1101 and the variable light (VL) domain sequence comprises a sequence having at least 90% identity to the VL domain sequence of antibody YANG-1101, provided that the antibody has the CDRs of antibody YANG-1101.

In one embodiment, the variable heavy (VH) domain sequence comprises a sequence having at least 90% identity to the VH domain sequence of antibody YANG-1103 and the variable light (VL) domain sequence comprises a sequence having at least 90% identity to the VL domain sequence of antibody YANG-1103, provided that the antibody has the CDRs of antibody YANG-1103.

In one embodiment, the variable heavy (VH) domain sequence comprises a sequence having at least 90% identity to the VH domain sequence of antibody YANG-1105 and the variable light (VL) domain sequence comprises a sequence having at least 90% identity to the VL domain sequence of antibody YANG-1105, provided that the antibody has the CDRs of antibody YANG-1105.

In one embodiment, the variable heavy (VH) domain sequence comprises a sequence having at least 90% identity to the VH domain sequence of antibody YANG-1106 and the variable light (VL) domain sequence comprises a sequence having at least 90% identity to the VL domain sequence of antibody YANG-1106, provided that the antibody has the CDRs of antibody YANG-1106.

In one embodiment, the variable heavy (VH) domain sequence comprises a sequence having at least 90% identity to the VH domain sequence of antibody YANG-1107 and the variable light (VL) domain sequence comprises a sequence having at least 90% identity to the VL domain sequence of antibody YANG-1107, provided that the antibody has the CDRs of antibody YANG-1107.

In one embodiment, the variable heavy (VH) domain sequence comprises a sequence having at least 90% identity to the VH domain sequence of antibody YANG-1108 and the variable light (VL) domain sequence comprises a sequence having at least 90% identity to the VL domain sequence of antibody YANG-1108, provided that the antibody has the CDRs of antibody YANG-1108.

In one embodiment, the variable heavy (VH) domain sequence comprises a sequence having at least 90% identity to the VH domain sequence of antibody YANG-1109 and the variable light (VL) domain sequence comprises a sequence having at least 90% identity to the VL domain sequence of antibody YANG-1109, provided that the antibody has the CDRs of antibody YANG-1109.

In one embodiment, the variable heavy (VH) domain sequence comprises a sequence having at least 90% identity to the VH domain sequence of antibody YANG-1110 and the variable light (VL) domain sequence comprises a sequence having at least 90% identity to the VL domain sequence of antibody YANG-1110, provided that the antibody has the CDRs of antibody YANG-1110.

In one embodiment, the variable heavy (VH) domain sequence comprises a sequence having at least 90% identity to the VH domain sequence of antibody YANG-1112 and the variable light (VL) domain sequence comprises a sequence having at least 90% identity to the VL domain sequence of antibody YANG-1112, provided that the antibody has the CDRs of antibody YANG-1112.

In one embodiment, the variable heavy (VH) domain sequence comprises a sequence having at least 90% identity to the VH domain sequence of antibody YANG-1113 and the variable light (VL) domain sequence comprises a sequence having at least 90% identity to the VL domain sequence of antibody YANG-1113, provided that the antibody has the CDRs of antibody YANG-1113.

In one embodiment, the variable heavy (VH) domain sequence comprises a sequence having at least 90% identity to the VH domain sequence of antibody YANG-1114 and the variable light (VL) domain sequence comprises a sequence having at least 90% identity to the VL domain sequence of antibody YANG-1114, provided that the antibody has the CDRs of antibody YANG-1114.

In one embodiment, the variable heavy (VH) domain sequence comprises a sequence having at least 90% identity to the VH domain sequence of antibody YANG-1115 and the variable light (VL) domain sequence comprises a sequence having at least 90% identity to the VL domain sequence of antibody YANG-1115, provided that the antibody has the CDRs of antibody YANG-1115.

In one embodiment, the variable heavy (VH) domain sequence comprises a sequence having at least 90% identity to the VH domain sequence of antibody YANG-1116 and the variable light (VL) domain sequence comprises a sequence having at least 90% identity to the VL domain sequence of antibody YANG-1116, provided that the antibody has the CDRs of antibody YANG-1116.

In one embodiment, the variable heavy (VH) domain sequence comprises a sequence having at least 90% identity to the VH domain sequence of antibody YANG-1117 and the variable light (VL) domain sequence comprises a sequence having at least 90% identity to the VL domain sequence of antibody YANG-1117, provided that the antibody has the CDRs of antibody YANG-1117.

In one embodiment, the variable heavy (VH) domain sequence comprises a sequence having at least 90% identity to the VH domain sequence of antibody YANG-1118 and the variable light (VL) domain sequence comprises a sequence having at least 90% identity to the VL domain sequence of antibody YANG-1118, provided that the antibody has the CDRs of antibody YANG-1118.

In one embodiment, the variable heavy (VH) domain sequence comprises a sequence having at least 90% identity to the VH domain sequence of antibody YANG-1119 and the variable light (VL) domain sequence comprises a sequence having at least 90% identity to the VL domain sequence of antibody YANG-1119, provided that the antibody has the CDRs of antibody YANG-1119.

In one embodiment, the variable heavy (VH) domain sequence comprises a sequence having at least 90% identity to the VH domain sequence of antibody YANG-2101 and the variable light (VL) domain sequence comprises a sequence having at least 90% identity to the VL domain sequence of antibody YANG-2101, provided that the antibody has the CDRs of antibody YANG-2101.

In one embodiment, the variable heavy (VH) domain sequence comprises a sequence having at least 90% identity to the VH domain sequence of antibody YANG-2102 and the variable light (VL) domain sequence comprises a sequence having at least 90% identity to the VL domain sequence of antibody YANG-2102, provided that the antibody has the CDRs of antibody YANG-2102.

In one embodiment, the variable heavy (VH) domain sequence comprises a sequence having at least 90% identity to the VH domain sequence of antibody YANG-2103 and the variable light (VL) domain sequence comprises a sequence having at least 90% identity to the VL domain sequence of antibody YANG-2103, provided that the antibody has the CDRs of antibody YANG-2103.

In one embodiment, the variable heavy (VH) domain sequence comprises a sequence having at least 90% identity to the VH domain sequence of antibody YANG-2104 and the variable light (VL) domain sequence comprises a sequence having at least 90% identity to the VL domain sequence of antibody YANG-2104, provided that the antibody has the CDRs of antibody YANG-2104.

In one embodiment, the variable heavy (VH) domain sequence comprises a sequence having at least 90% identity to the VH domain sequence of antibody YANG-2105 and the variable light (VL) domain sequence comprises a sequence having at least 90% identity to the VL domain sequence of antibody YANG-2105, provided that the antibody has the CDRs of antibody YANG-2105.

In one embodiment, the variable heavy (VH) domain sequence comprises a sequence having at least 90% identity to the VH domain sequence of antibody YANG-2106 and the variable light (VL) domain sequence comprises a sequence having at least 90% identity to the VL domain sequence of antibody YANG-2106, provided that the antibody has the CDRs of antibody YANG-2106.

In one embodiment, the variable heavy (VH) domain sequence comprises a sequence having at least 90% identity to the VH domain sequence of antibody YANG-2107 and the variable light (VL) domain sequence comprises a sequence having at least 90% identity to the VL domain sequence of antibody YANG-2107, provided that the antibody has the CDRs of antibody YANG-2107.

In one embodiment, the variable heavy (VH) domain sequence comprises a sequence having at least 90% identity to the VH domain sequence of antibody YANG-2108 and the variable light (VL) domain sequence comprises a sequence having at least 90% identity to the VL domain sequence of antibody YANG-2108, provided that the antibody has the CDRs of antibody YANG-2108.

In one embodiment, the variable heavy (VH) domain sequence comprises a sequence having at least 90% identity to the VH domain sequence of antibody YANG-2109 and the variable light (VL) domain sequence comprises a sequence having at least 90% identity to the VL domain sequence of antibody YANG-2109, provided that the antibody has the CDRs of antibody YANG-2109.

In one embodiment, the variable heavy (VH) domain sequence comprises a sequence having at least 90% identity to the VH domain sequence of antibody YANG-2110 and the variable light (VL) domain sequence comprises a sequence having at least 90% identity to the VL domain sequence of antibody YANG-2110, provided that the antibody has the CDRs of antibody YANG-2110.

In one embodiment, the variable heavy (VH) domain sequence comprises a sequence having at least 90% identity to the VH domain sequence of antibody YANG-2111 and the variable light (VL) domain sequence comprises a sequence having at least 90% identity to the VL domain sequence of antibody YANG-2111, provided that the antibody has the CDRs of antibody YANG-2111.

In one embodiment, the antibody specifically binds to the RBD of the SARS-CoV-2 spike protein and competes for binding to the SARS-CoV-2 spike protein with the human ACE2 receptor, and the antibody comprises a variable heavy (VH) domain sequence and a variable light (VL) domain sequence and wherein the variable heavy (VH) domain and variable light (VL) domain sequences respectively comprise a sequence having at least 90% identity to the variable heavy (VH) and variable light (VL) domain sequences of antibody YANG-1101, YANG-1103, YANG-1105, YANG-1106, YANG-1107, YANG-1108, YANG-1109, YANG-1110, YANG-1112, YANG-1112a, YANG-1112b, YANG-1112c, YANG-1113, YANG-1114, YANG-1115, YANG-1116, YANG-1117, YANG-1118, YANG-1119, YANG-2101, YANG-2102, YANG-2103, YANG-2104, YANG-2105, YANG-2106, YANG-2107, YANG-2108, YANG-2108a, YANG-2108b, YANG-2108c, YANG-2108d, YANG-2108e, YANG-2108f, YANG-2108g, YANG-2108h, YANG-2108i, YANG-2108j, YANG-2108k, YANG-21081, YANG-2109, YANG-2110, or YANG-2111 provided that the antibody has the CDRs of antibody YANG-1101, YANG-1103, YANG-1105, YANG-1106, YANG-1107, YANG-1108, YANG-1109, YANG-1110, YANG-1112, YANG-1112a, YANG-1112b, YANG-1112c, YANG-1113, YANG-1114, YANG-1115, YANG-1116, YANG-1117, YANG-1118, YANG-1119, YANG-2101, YANG-2102, YANG-2103, YANG-2104, YANG-2105, YANG-2106, YANG-2107, YANG-2108, YANG-2108a, YANG-2108b, YANG-2108c, YANG-2108d, YANG-2108e, YANG-2108f, YANG-2108g, YANG-2108h, YANG-2108i, YANG-2108j, YANG-2108k, YANG-21081, YANG-2109, YANG-2110, orYANG-2111, YANG-2111a, YANG-2111b, respectively.

In one embodiment, the antibody specifically binds to the RBD of the SARS-CoV-2 spike protein with a KD of 0.1 nM or lower (e.g. as measured by surface plasmon resonance (SPR)) and competes for binding to the SARS-CoV-2 spike protein with the human ACE2 receptor, and the antibody comprises a variable heavy (VH) domain sequence and a variable light (VL) domain sequence and wherein the variable heavy (VH) domain and variable light (VL) domain sequences respectively comprise a sequence having at least 90% identity to the variable heavy (VH) and variable light (VL) domain sequences of antibody YANG-1101, YANG-1103, YANG-1105, YANG-1106, YANG-1107, YANG-1108, YANG-1109, YANG-1110, YANG-1112, YANG-1112a, YANG-1112b, YANG-1112c, YANG-1113, YANG-1114, YANG-1115, YANG-1116, YANG-1117, YANG-1118, YANG-1119, YANG-2101, YANG-2102, YANG-2103, YANG-2104, YANG-2105, YANG-2106, YANG-2107, YANG-2108, YANG-2108a, YANG-2108b, YANG-2108c, YANG-2108d, YANG-2108e, YANG-2108f, YANG-2108g, YANG-2108h, YANG-2108i, YANG-2108j, YANG-2108k, YANG-21081, YANG-2109, YANG-2110, or YANG-2111 provided that the antibody has the CDRs of antibody YANG-1101, YANG-1103, YANG-1105, YANG-1106, YANG-1107, YANG-1108, YANG-1109, YANG-1110, YANG-1112, YANG-1112a, YANG-1112b, YANG-1112c, YANG-1113, YANG-1114, YANG-1115, YANG-1116, YANG-1117, YANG-1118, YANG-1119, YANG-2101, YANG-2102, YANG-2103, YANG-2104, YANG-2105, YANG-2106, YANG-2107, YANG-2108, YANG-2108a, YANG-2108b, YANG-2108c, YANG-2108d, YANG-2108e, YANG-2108f, YANG-2108g, YANG-2108h, YANG-2108i, YANG-2108j, YANG-2108k, YANG-21081, YANG-2109, YANG-2110, or YANG-2111, YANG-2111a, YANG-2111b, respectively.

In one embodiment, the antibody specifically binds to the RBD of the SARS-CoV-2 spike protein and competes for binding to the SARS-CoV-2 spike protein with the human ACE2 receptor, and the antibody comprises a variable heavy (VH) domain sequence and a variable light (VL) domain sequence and wherein the variable heavy (VH) domain and variable light (VL) domain sequences respectively comprise a sequence having at least 90% identity to the variable heavy (VH) and variable light (VL) domain sequences of antibody YANG-1112, YANG-2107, YANG-2108, or YANG-2111, provided that the antibody has the CDRs of antibody YANG-1112, YANG-2107, YANG-2108, or YANG-2111, respectively.

In one embodiment, the antibody specifically binds to the RBD of the SARS-CoV-2 spike protein with a KD of 0.1 nM or lower (e.g. as measured by surface plasmon resonance (SPR)) and competes for binding to the SARS-CoV-2 spike protein with the human ACE2 receptor, and the antibody comprises a variable heavy (VH) domain sequence and a variable light (VL) domain sequence and wherein the variable heavy (VH) domain and variable light (VL) domain sequences respectively comprise a sequence having at least 90% identity to the variable heavy (VH) and variable light (VL) domain sequences of antibody YANG-1112, YANG-2107, YANG-2108, or YANG-2111, provided that the antibody has the CDRs of antibody YANG-1112, YANG-2107, YANG-2108, or YANG-2111, respectively.

In one embodiment, the antibody specifically binds to the S2 subunit of the SARS-CoV-2 spike protein and competes for binding to the SARS-CoV-2 spike protein with the human ACE2 receptor and neutralises SARS-CoV-2 with at least 60% neutralisation (e.g. as measured in a pseudovirus neutralisation assay), and the antibody comprises a variable heavy (VH) domain sequence and a variable light (VL) domain sequence and wherein the variable heavy (VH) domain and variable light (VL) domain sequences respectively comprise a sequence having at least 90% identity to the variable heavy (VH) and variable light (VL) domain sequences of antibody YANG-1112, YANG-2107, YANG-2108, or YANG-2111, provided that the antibody has the CDRs of antibody YANG-1112, YANG-2107, YANG-2108, or YANG-2111, respectively.

In one embodiment, the antibody specifically binds to the S2 subunit of the SARS-CoV-2 spike protein with a KD of 0.1 nM or lower (e.g. as measured by surface plasmon resonance (SPR)) and competes for binding to the SARS-CoV-2 spike protein with the human ACE2 receptor and neutralises SARS-CoV-2 with at least 60% neutralisation (e.g. as measured in a pseudovirus neutralisation assay), and the antibody comprises a variable heavy (VH) domain sequence and a variable light (VL) domain sequence and wherein the variable heavy (VH) domain and variable light (VL) domain sequences respectively comprise a sequence having at least 90% identity to the variable heavy (VH) and variable light (VL) domain sequences of antibody YANG-1112, YANG-2107, YANG-2108, or YANG-2111, provided that the antibody has the CDRs of antibody YANG-1112, YANG-2107, YANG-2108, or YANG-2111, respectively.

In one embodiment, the present invention provides an antibody specifically binds to the RBD of the SARS-CoV-2 spike protein and competes for binding to the SARS-CoV-2 spike protein with the human ACE2 receptor, wherein the antibody comprises a variable heavy (VH) domain sequence and a variable light (VL) domain sequence and wherein the variable heavy (VH) domain and variable light (VL) domain sequences respectively comprise the variable heavy (VH) and variable light (VL) domain sequences of antibody YANG-1101, YANG-1103, YANG-1105, YANG-1106, YANG-1107, YANG-1108, YANG-1109, YANG-1110, YANG-1112, YANG-1112a, YANG-1112b, YANG-1112c, YANG-1113, YANG-1114, YANG-1115, YANG-1116, YANG-1117, YANG-1118, YANG-1119, YANG-2101, YANG-2102, YANG-2103, YANG-2104, YANG-2105, YANG-2106, YANG-2107, YANG-2108, YANG-2108a, YANG-2108b, YANG-2108c, YANG-2108d, YANG-2108e, YANG-2108f, YANG-2108g, YANG-2108h, YANG-2108i, YANG-2108j, YANG-2108k, YANG-21081, YANG-2109, YANG-2110, or YANG-2111.

In one embodiment, the present invention provides an antibody specifically binds to the RBD of the SARS-CoV-2 spike protein and competes for binding to the SARS-CoV-2 spike protein with the human ACE2 receptor, wherein the antibody comprises a variable heavy (VH) domain sequence and a variable light (VL) domain sequence and wherein the variable heavy (VH) domain and variable light (VL) domain sequences respectively comprise the variable heavy (VH) and variable light (VL) domain sequences of antibody YANG-1112, YANG-2107, YANG-2108, or YANG-2111.

An antibody that specifically binds to the receptor binding domain (RBD) of the SARS-CoV-2 spike protein, wherein the antibody comprises the VH and VL domain sequences of antibody YANG-1101.

An antibody that specifically binds to the receptor binding domain (RBD) of the SARS-CoV-2 spike protein, wherein the antibody comprises the VH and VL domain sequences of antibody YANG-1103.

An antibody that specifically binds to the receptor binding domain (RBD) of the SARS-CoV-2 spike protein, wherein the antibody comprises the VH and VL domain sequences of antibody YANG-1105.

An antibody that specifically binds to the receptor binding domain (RBD) of the SARS-CoV-2 spike protein, wherein the antibody comprises the VH and VL domain sequences of antibody YANG-1106.

An antibody that specifically binds to the receptor binding domain (RBD) of the SARS-CoV-2 spike protein, wherein the antibody comprises the VH and VL domain sequences of antibody YANG-1107.

An antibody that specifically binds to the receptor binding domain (RBD) of the SARS-CoV-2 spike protein, wherein the antibody comprises the VH and VL domain sequences of antibody YANG-1108.

An antibody that specifically binds to the receptor binding domain (RBD) of the SARS-CoV-2 spike protein, wherein the antibody comprises the VH and VL domain sequences of antibody YANG-1109.

An antibody that specifically binds to the receptor binding domain (RBD) of the SARS-CoV-2 spike protein, wherein the antibody comprises the VH and VL domain sequences of antibody YANG-1110.

An antibody that specifically binds to the receptor binding domain (RBD) of the SARS-CoV-2 spike protein, wherein the antibody comprises the VH and VL domain sequences of antibody YANG-1112.

An antibody that specifically binds to the receptor binding domain (RBD) of the SARS-CoV-2 spike protein, wherein the antibody comprises the VH and VL domain sequences of antibody YANG-1113.

An antibody that specifically binds to the receptor binding domain (RBD) of the SARS-CoV-2 spike protein, wherein the antibody comprises the VH and VL domain sequences of antibody YANG-1114.

An antibody that specifically binds to the receptor binding domain (RBD) of the SARS-CoV-2 spike protein, wherein the antibody comprises the VH and VL domain sequences of antibody YANG-1115.

An antibody that specifically binds to the receptor binding domain (RBD) of the SARS-CoV-2 spike protein, wherein the antibody comprises the VH and VL domain sequences of antibody YANG-1116.

An antibody that specifically binds to the receptor binding domain (RBD) of the SARS-CoV-2 spike protein, wherein the antibody comprises the VH and VL domain sequences of antibody YANG-1117.

An antibody that specifically binds to the receptor binding domain (RBD) of the SARS-CoV-2 spike protein, wherein the antibody comprises the VH and VL domain sequences of antibody YANG-1118.

An antibody that specifically binds to the receptor binding domain (RBD) of the SARS-CoV-2 spike protein, wherein the antibody comprises the VH and VL domain sequences of antibody YANG-1119.

An antibody that specifically binds to the receptor binding domain (RBD) of the SARS-CoV-2 spike protein, wherein the antibody comprises the VH and VL domain sequences of antibody YANG-2101.

An antibody that specifically binds to the receptor binding domain (RBD) of the SARS-CoV-2 spike protein, wherein the antibody comprises the VH and VL domain sequences of antibody YANG-2102.

An antibody that specifically binds to the receptor binding domain (RBD) of the SARS-CoV-2 spike protein, wherein the antibody comprises the VH and VL domain sequences of antibody YANG-2103.

An antibody that specifically binds to the receptor binding domain (RBD) of the SARS-CoV-2 spike protein, wherein the antibody comprises the VH and VL domain sequences of antibody YANG-2104.

An antibody that specifically binds to the receptor binding domain (RBD) of the SARS-CoV-2 spike protein, wherein the antibody comprises the VH and VL domain sequences of antibody YANG-2105.

An antibody that specifically binds to the receptor binding domain (RBD) of the SARS-CoV-2 spike protein, wherein the antibody comprises the VH and VL domain sequences of antibody YANG-2106.

An antibody that specifically binds to the receptor binding domain (RBD) of the SARS-CoV-2 spike protein, wherein the antibody comprises the VH and VL domain sequences of antibody YANG-2107.

An antibody that specifically binds to the receptor binding domain (RBD) of the SARS-CoV-2 spike protein, wherein the antibody comprises the VH and VL domain sequences of antibody YANG-2108.

An antibody that specifically binds to the receptor binding domain (RBD) of the SARS-CoV-2 spike protein, wherein the antibody comprises the VH and VL domain sequences of antibody YANG-2109.

An antibody that specifically binds to the receptor binding domain (RBD) of the SARS-CoV-2 spike protein, wherein the antibody comprises the VH and VL domain sequences of antibody YANG-2110.

An antibody that specifically binds to the receptor binding domain (RBD) of the SARS-CoV-2 spike protein, wherein the antibody comprises the VH and VL domain sequences of antibody YANG-2111.

In one embodiment, the antibody comprises a variable heavy (VH) domain sequence and a variable light (VL) domain sequence and wherein the variable heavy (VH) domain and variable light (VL) domain sequences respectively comprise the variable heavy (VH) and variable light (VL) domain sequences of antibody YANG-1101, YANG-1103, YANG-1105, YANG-1106, YANG-1107, YANG-1108, YANG-1109, YANG-1110, YANG-1112, YANG-1112a, YANG-1112b, YANG-1112c, YANG-1113, YANG-1114, YANG-1115, YANG-1116, YANG-1117, YANG-1118, YANG-1119, YANG-2101, YANG-2102, YANG-2103, YANG-2104, YANG-2105, YANG-2106, YANG-2107, YANG-2108, YANG-2108a, YANG-2108b, YANG-2108c, YANG-2108d, YANG-2108e, YANG-2108f, YANG-2108g, YANG-2108h, YANG-2108i, YANG-2108j, YANG-2108k, YANG-21081, YANG-2109, YANG-2110, or YANG-2111.

In one embodiment, the antibody comprises a variable heavy (VH) domain sequence and a variable light (VL) domain sequence and wherein the variable heavy (VH) domain and variable light (VL) domain sequences respectively comprise the variable heavy (VH) and variable light (VL) domain sequences of antibody YANG-1112, YANG-2107, YANG-2108, or YANG-2111.

In one embodiment, the antibody comprises the VH and VL domain sequences of antibody YANG-1101.

In one embodiment, the antibody comprises the VH and VL domain sequences of antibody YANG-1103.

In one embodiment, the antibody comprises the VH and VL domain sequences of antibody YANG-1105.

In one embodiment, the antibody comprises the VH and VL domain sequences of antibody YANG-1106.

In one embodiment, the antibody comprises the VH and VL domain sequences of antibody YANG-1107.

In one embodiment, the antibody comprises the VH and VL domain sequences of antibody YANG-1108.

In one embodiment, the antibody comprises the VH and VL domain sequences of antibody YANG-1109.

In one embodiment, the antibody comprises the VH and VL domain sequences of antibody YANG-1110.

In one embodiment, the antibody comprises the VH and VL domain sequences of antibody YANG-1112.

In one embodiment, the antibody comprises the VH and VL domain sequences of antibody YANG-1113.

In one embodiment, the antibody comprises the VH and VL domain sequences of antibody YANG-1114.

In one embodiment, the antibody comprises the VH and VL domain sequences of antibody YANG-1115.

In one embodiment, the antibody comprises the VH and VL domain sequences of antibody YANG-1116.

In one embodiment, the antibody comprises the VH and VL domain sequences of antibody YANG-1117.

In one embodiment, the antibody comprises the VH and VL domain sequences of antibody YANG-1118.

In one embodiment, the antibody comprises the VH and VL domain sequences of antibody YANG-1119.

In one embodiment, the antibody comprises the VH and VL domain sequences of antibody YANG-2101.

In one embodiment, the antibody comprises the VH and VL domain sequences of antibody YANG-2102.

In one embodiment, the antibody comprises the VH and VL domain sequences of antibody YANG-2103.

In one embodiment, the antibody comprises the VH and VL domain sequences of antibody YANG-2104.

In one embodiment, the antibody comprises the VH and VL domain sequences of antibody YANG-2105.

In one embodiment, the antibody comprises the VH and VL domain sequences of antibody YANG-2106.

In one embodiment, the antibody comprises the VH and VL domain sequences of antibody YANG-2107.

In one embodiment, the antibody comprises the VH and VL domain sequences of antibody YANG-2108.

In one embodiment, the antibody comprises the VH and VL domain sequences of antibody YANG-2109.

In one embodiment, the antibody comprises the VH and VL domain sequences of antibody YANG-2110.

In one embodiment, the antibody comprises the VH and VL domain sequences of antibody YANG-2111.

Antibodies are provided which bind to the same epitope on the RBD of the SARS-CoV-2 spike protein as an antibody described anywhere herein.

An antibody is provided which bind to the same epitope as antibody YANG-1101, YANG-1103, YANG-1105, YANG-1106, YANG-1107, YANG-1108, YANG-1109, YANG-1110, YANG-1112, YANG-1112a, YANG-1112b, YANG-1112c, YANG-1113, YANG-1114, YANG-1115, YANG-1116, YANG-1117, YANG-1118, YANG-1119, YANG-2101, YANG-2102, YANG-2103, YANG-2104, YANG-2105, YANG-2106, YANG-2107, YANG-2108, YANG-2108a, YANG-2108b, YANG-2108c, YANG-2108d, YANG-2108e, YANG-2108f, YANG-2108g, YANG-2108h, YANG-2108i, YANG-2108j, YANG-2108k, YANG-21081, YANG-2109, YANG-2110, orYANG-2111, YANG-2111a, YANG-2111b, e.g. as defined by its VH and VL sequences.

An antibody is provided which bind to the same epitope as antibody YANG-1112, YANG-2107, YANG-2108, or YANG-2111, e.g. as defined by its VH and VL sequences.

In one embodiment, an antibody is provided which binds to the same epitope as antibody YANG-1101.

In one embodiment, an antibody is provided which binds to the same epitope as antibody YANG-1103.

In one embodiment, an antibody is provided which binds to the same epitope as antibody YANG-1105.

In one embodiment, an antibody is provided which binds to the same epitope as antibody YANG-1106.

In one embodiment, an antibody is provided which binds to the same epitope as antibody YANG-1107.

In one embodiment, an antibody is provided which binds to the same epitope as antibody YANG-1108.

In one embodiment, an antibody is provided which binds to the same epitope as antibody YANG-1109.

In one embodiment, an antibody is provided which binds to the same epitope as antibody YANG-1110.

In one embodiment, an antibody is provided which binds to the same epitope as antibody YANG-1112.

In one embodiment, an antibody is provided which binds to the same epitope as antibody YANG-1113.

In one embodiment, an antibody is provided which binds to the same epitope as antibody YANG-1114.

In one embodiment, an antibody is provided which binds to the same epitope as antibody YANG-1115.

In one embodiment, an antibody is provided which binds to the same epitope as antibody YANG-1116.

In one embodiment, an antibody is provided which binds to the same epitope as antibody YANG-1117.

In one embodiment, an antibody is provided which binds to the same epitope as antibody YANG-1118.

In one embodiment, an antibody is provided which binds to the same epitope as antibody YANG-1119.

In one embodiment, an antibody is provided which binds to the same epitope as antibody YANG-2101.

In one embodiment, an antibody is provided which binds to the same epitope as antibody YANG-2102.

In one embodiment, an antibody is provided which binds to the same epitope as antibody YANG-2103.

In one embodiment, an antibody is provided which binds to the same epitope as antibody YANG-2104.

In one embodiment, an antibody is provided which binds to the same epitope as antibody YANG-2105.

In one embodiment, an antibody is provided which binds to the same epitope as antibody YANG-2106.

In one embodiment, an antibody is provided which binds to the same epitope as antibody YANG-2107.

In one embodiment, an antibody is provided which binds to the same epitope as antibody YANG-2108.

In one embodiment, an antibody is provided which binds to the same epitope as antibody YANG-2109.

In one embodiment, an antibody is provided which binds to the same epitope as antibody YANG-2110.

In one embodiment, an antibody is provided which binds to the same epitope as antibody YANG-2111.

An antibody may contact the SARS-CoV-2 spike protein with a footprint that fully or partly overlaps with that of an antibody disclosed anywhere herein. As described elsewhere herein, competition between antibodies may be determined, for example using SPR, and antibodies are provided which compete for binding to the spike protein (compete for binding to their epitope) with an IgG antibody as described anywhere herein.

An antibody of the present invention may be one which competes for binding to SARS-CoV-2 spike protein with any anti-RBD antibody described herein, such as YANG-1101, YANG-1103, YANG-1105, YANG-1106, YANG-1107, YANG-1108, YANG-1109, YANG-1110, YANG-1112, YANG-1112a, YANG-1112b, YANG-1112c, YANG-1113, YANG-1114, YANG-1115, YANG-1116, YANG-1117, YANG-1118, YANG-1119, YANG-2101, YANG-2102, YANG-2103, YANG-2104, YANG-2105, YANG-2106, YANG-2107, YANG-2108, YANG-2108a, YANG-2108b, YANG-2108c, YANG-2108d, YANG-2108e, YANG-2108f, YANG-2108g, YANG-2108h, YANG-2108i, YANG-2108j, YANG-2108k, YANG-21081, YANG-2109, YANG-2110, orYANG-2111, YANG-2111a, YANG-2111b, e.g. as defined by its VH and VL sequences.

An antibody of the present invention may be one which competes for binding to the SARS-CoV-2 spike protein with antibody YANG-1101.

An antibody of the present invention may be one which competes for binding to the SARS-CoV-2 spike protein with antibody YANG-1103.

An antibody of the present invention may be one which competes for binding to the SARS-CoV-2 spike protein with antibody YANG-1105.

An antibody of the present invention may be one which competes for binding to the SARS-CoV-2 spike protein with antibody YANG-1106.

An antibody of the present invention may be one which competes for binding to the SARS-CoV-2 spike protein with antibody YANG-1107.

An antibody of the present invention may be one which competes for binding to the SARS-CoV-2 spike protein with antibody YANG-1108.

An antibody of the present invention may be one which competes for binding to the SARS-CoV-2 spike protein with antibody YANG-1109.

An antibody of the present invention may be one which competes for binding to the SARS-CoV-2 spike protein with antibody YANG-1110.

An antibody of the present invention may be one which competes for binding to the SARS-CoV-2 spike protein with antibody YANG-1112.

An antibody of the present invention may be one which competes for binding to the SARS-CoV-2 spike protein with antibody YANG-1113.

An antibody of the present invention may be one which competes for binding to the SARS-CoV-2 spike protein with antibody YANG-1114.

An antibody of the present invention may be one which competes for binding to the SARS-CoV-2 spike protein with antibody YANG-1115.

An antibody of the present invention may be one which competes for binding to the SARS-CoV-2 spike protein with antibody YANG-1116.

An antibody of the present invention may be one which competes for binding to the SARS-CoV-2 spike protein with antibody YANG-1117.

An antibody of the present invention may be one which competes for binding to the SARS-CoV-2 spike protein with antibody YANG-1118.

An antibody of the present invention may be one which competes for binding to the SARS-CoV-2 spike protein with antibody YANG-1119.

An antibody of the present invention may be one which competes for binding to the SARS-CoV-2 spike protein with antibody YANG-2101.

An antibody of the present invention may be one which competes for binding to the SARS-CoV-2 spike protein with antibody YANG-2102.

An antibody of the present invention may be one which competes for binding to the SARS-CoV-2 spike protein with antibody YANG-2103.

An antibody of the present invention may be one which competes for binding to the SARS-CoV-2 spike protein with antibody YANG-2104.

An antibody of the present invention may be one which competes for binding to the SARS-CoV-2 spike protein with antibody YANG-2105.

An antibody of the present invention may be one which competes for binding to the SARS-CoV-2 spike protein with antibody YANG-2106.

An antibody of the present invention may be one which competes for binding to the SARS-CoV-2 spike protein with antibody YANG-2107.

An antibody of the present invention may be one which competes for binding to the SARS-CoV-2 spike protein with antibody YANG-2108.

An antibody of the present invention may be one which competes for binding to the SARS-CoV-2 spike protein with antibody YANG-2109.

An antibody of the present invention may be one which competes for binding to the SARS-CoV-2 spike protein with antibody YANG-2110.

An antibody of the present invention may be one which competes for binding to the SARS-CoV-2 spike protein with antibody YANG-2111.

In one embodiment, the antibody comprises VH and/or VL domain framework regions of human germline gene segment sequences.

In one embodiment, the antibody comprises an antibody VH domain which
i) is derived from recombination of a human heavy chain V gene segment, a human heavy chain D gene segment and a human heavy chain J gene segment, wherein
   the V gene segment is IGHV4-39*01, IGHV3-53*01, IGHV3-30*18, IGHV2-5*10, IGHV3-21*03, IGHV3-13*01, IGHV3-33*01, IGHV4-4*02, IGHV1-69*05 or IGHV3-9*01; and/or the J gene segment is IGHJ3*02, IGHJ4*02 or IGHJ6*02; or
ii) comprises framework regions FR1, FR2, FR3 and FR4, wherein
   FR1 aligns with human germline V gene segment IGHV4-39*01, IGHV3-53*01, IGHV3-30*18,
IGHV2-5*10, IGHV3-21*03, IGHV3-13*01, IGHV3-33*01, IGHV4-4*02, IGHV1-69*05 orIGHV3-9*01 with up to 1, 2, 3, 4, or 5 amino acid alterations,
   FR2 aligns with human germline V gene segment IGHV4-39*01, IGHV3-53*01, IGHV3-30*18, IGHV2-5*10, IGHV3-21*03, IGHV3-13*01, IGHV3-33*01, IGHV4-4*02, IGHV1-69*05 orIGHV3-9*01 with up to 1, 2, 3, 4, or 5 amino acid alterations,
   FR3 aligns with human germline V gene segment IGHV4-39*01, IGHV3-53*01, IGHV3-30*18, IGHV2-5*10, IGHV3-21*03, IGHV3-13*01, IGHV3-33*01, IGHV4-4*02, IGHV1-69*05 orIGHV3-9*01 with up to 1, 2, 3, 4, or 5 amino acid alterations, and/or
   FR4 aligns with human germline J gene segment IGHJ3*02, IGHJ4*02 or IGHJ6*02 with up to 1, 2, 3, 4 or 5 amino acid alterations.

In one embodiment, the antibody comprises an antibody VH domain which
i) is derived from recombination of a human heavy chain V gene segment IGHV4-39*01, IGHV3-53*01, IGHV3-30*18, IGHV2-5*10, IGHV3-21*03, IGHV3-13*01, IGHV3-33*01, IGHV4-4*02, IGHV1-69*05 or IGHV3-9*01, a human heavy chain D gene segment and a human heavy chain J gene segment, or
ii) comprises framework regions FR1, FR2, FR3 and FR4, wherein FR1, FR2 and FR3 each align with human germline V segment IGHV4-39*01, IGHV3-53*01, IGHV3-30*18, IGHV2-5*10, IGHV3-21*03, IGHV3-13*01, IGHV3-33*01, IGHV4-4*02, IGHV1-69*05 or IGHV3-9*01 with up to 1, 2, 3, 4 or 5 amino acid alterations.

In one embodiment, the J gene segment is IGHJ3*02, IGHJ4*02 or IGHJ6*02, or the VH domain framework region FR4 aligns with human germline J gene segment IGHJ3*02, IGHJ4*02 or IGHJ6*02 with 1, 2, 3, 4 or 5 amino acid alterations.

In one embodiment, the antibody comprises an antibody VL domain which
i) is derived from recombination of a human light chain V gene segment and a human light chain J gene segment, wherein
   the V gene segment is IGLV2-23*d02, IGKV1-9*d01, IGKV3-15*01, IGKV1D-13*d01, IGLV4-60*d03, IGLV3-10*01, IGLV2-14*01, IGKV1-16*02, IGLV3-21*d01, IGKV1D-17*01, IGKV1-17*01, IGKV3D-7*01 or IGKV2-28*01; and/or the J gene segment is IGLJ2*01, IGKJ5*01, IGKJ2*04, IGKJ1*01, IGKJ4*01 or IGLJ3*02; or
ii) comprises framework regions FR1, FR2, FR3 and FR4, wherein
   FR1 aligns with human germline V gene segment IGLV2-23*d02, IGKV1-9*d01, IGKV3-15*01, IGKV1D-13*d01, IGLV4-60*d03, IGLV3-10*01, IGLV2-14*01, IGKV1-16*02, IGLV3-21*d01, IGKV1D-17*01, IGKV1-17*01, IGKV3D-7*01 or IGKV2-28*01 with up to 1, 2, 3, 4, or 5 amino acid alterations,
   FR2 aligns with human germline V gene segment IGLV2-23*d02, IGKV1-9*d01, IGKV3-15*01, IGKV1D-13*d01, IGLV4-60*d03, IGLV3-10*01, IGLV2-14*01, IGKV1-16*02, IGLV3-21*d01, IGKV1D-17*01, IGKV1-17*01, IGKV3D-7*01 or IGKV2-28*01 with up to 1, 2, 3, 4, or 5 amino acid alterations
   FR3 aligns with human germline V gene segment IGLV2-23*d02, IGKV1-9*d01, IGKV3-15*01, IGKV1D-13*d01, IGLV4-60*d03, IGLV3-10*01, IGLV2-14*01, IGKV1-16*02, IGLV3-21*d01, IGKV1D-17*01, IGKV1-17*01, IGKV3D-7*01 or IGKV2-28*01 with up to 1, 2, 3, 4 or 5 amino acid alterations, and/or
   FR4 aligns with human germline J gene segment IGLJ2*01, IGKJ5*01, IGKJ2*04, IGKJ1*01, IGKJ4*01 or IGLJ3*02 with up to 1, 2, 3, 4 or 5 amino acid alterations.

In one embodiment, the antibody comprises an antibody VL domain derived from recombination of a human light chain V gene segment and a human light chain J gene segment, wherein:
the V gene segment is IGLV2-23*d02, IGKV1-9*d01, IGKV3-15*01, IGKV1D-13*d01, IGLV4-60*d03, IGLV3-10*01, IGLV2-14*01, IGKV1-16*02, IGLV3-21*d01, IGKV1D-17*01, IGKV1-17*01, IGKV3D-7*01 or IGKV2-28*01, and optionally
the J gene segment is IGLJ2*01, IGKJ5*01, IGKJ2*04, IGKJ1*01, IGKJ4*01 or IGLJ3*02.

Example combinations of v and j gene segments for heavy and light chain variable domains are shown in Table 3, and these represent preferred combinations. The heavy and light chain variable domains may optionally be derived from the v and j gene segments identified in Table 3 for any one individual YANG antibody.

### Group B—Trimer Binders:

In a second aspect, the present invention provides an antibody that preferentially binds to the trimer form of the SARS-CoV-2 spike protein over the isolated RBD domain, isolated S1 subunit or isolated S2 subunit of the SARS-CoV-2 spike protein.

In one embodiment, the antibody specifically binds to the trimer form of the SARS-CoV-2 spike protein and does not bind to the isolated RBD domain.

In one embodiment, the antibody specifically binds to the trimer form of the SARS-CoV-2 spike protein and does not bind to the isolated RBD domain, isolated S1 subunit or isolated S2 subunit of the SARS-CoV-2 spike protein.

In one embodiment, the antibody is a neutralising antibody.

In one embodiment, the antibody neutralises SARS-CoV-2 with an IC50 of 75 nM or lower, preferably 15 nM or lower (e.g. as measured in a pseudovirus neutralisation assay).

In one embodiment, the antibody comprises a variable heavy (VH) domain sequence comprising complementarity determining regions (CDRs) HCDR1, HCDR2 and HCDR3, and a variable light (VL) domain sequence comprising complementarity determining regions LCDR1, LCDR2 and LCDR3, wherein the HCDR3 is the HCDR3 of antibody IMPI-030, IMPI-053, IMPI-025, IMPI-040, IMPI-007, IMPI-020, IMPI-032, IMPI-023, IMPI-039, IMPI-001, IMPI-019, IMPI-010, IMPI-008, IMPI-031, IMPI-057, IMPI-022, IMPI-035, IMPI-067 or IMPI-072.

In one embodiment, the HCDR3 is the HCDR3 of antibody IMPI-030.

In one embodiment, the HCDR3 is the HCDR3 of antibody IMPI-053.

In one embodiment, the HCDR3 is the HCDR3 of antibody IMPI-025.

In one embodiment, the HCDR3 is the HCDR3 of antibody IMPI-040.

In one embodiment, the HCDR3 is the HCDR3 of antibody IMPI-007.

In one embodiment, the HCDR3 is the HCDR3 of antibody IMPI-020.

In one embodiment, the HCDR3 is the HCDR3 of antibody IMPI-032.

In one embodiment, the HCDR3 is the HCDR3 of antibody IMPI-023.

In one embodiment, the HCDR3 is the HCDR3 of antibody IMPI-039.

In one embodiment, the HCDR3 is the HCDR3 of antibody IMPI-001.

In one embodiment, the HCDR3 is the HCDR3 of antibody IMPI-019.

In one embodiment, the HCDR3 is the HCDR3 of antibody IMPI-010.

In one embodiment, the HCDR3 is the HCDR3 of antibody IMPI-008.

In one embodiment, the HCDR3 is the HCDR3 of antibody IMPI-031.

In one embodiment, the HCDR3 is the HCDR3 of antibody IMPI-057.

In one embodiment, the HCDR3 is the HCDR3 of antibody IMPI-022.

In one embodiment, the HCDR3 is the HCDR3 of antibody IMPI-035.

In one embodiment, the HCDR3 is the HCDR3 of antibody IMPI-067.

In one embodiment, the HCDR3 is the HCDR3 of antibody IMPI-072.

In one embodiment, the present invention provides anti-SARS-CoV2 antibody,

wherein the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3,
wherein the CDRs are those of antibody IMPI-030, IMPI-053, IMPI-025, IMPI-040, IMPI-007, IMPI-020, IMPI-032, IMPI-023, IMPI-039, IMPI-001, IMPI-019, IMPI-010, IMPI-008, IMPI-031, IMPI-057, IMPI-022, IMPI-035, IMPI-067 or IMPI-072.

In one embodiment, the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3, wherein the CDRs are those of antibody IMPI-030, IMPI-053, IMPI-025, IMPI-040, IMPI-007, IMPI-020, IMPI-032, IMPI-023, IMPI-039, IMPI-001, IMPI-019, IMPI-010, IMPI-008, IMPI-031, IMPI-057, IMPI-022, IMPI-035, IMPI-067 or IMPI-072.

In one embodiment, the antibody has the CDRs of antibody IMPI-030.

In one embodiment, the antibody has the CDRs of antibody IMPI-053.

In one embodiment, the antibody has the CDRs of antibody IMPI-025.

In one embodiment, the antibody has the CDRs of antibody IMPI-040.

In one embodiment, the antibody has the CDRs of antibody IMPI-007.

In one embodiment, the antibody has the CDRs of antibody IMPI-020.

In one embodiment, the antibody has the CDRs of antibody IMPI-032.

In one embodiment, the antibody has the CDRs of antibody IMPI-023.

In one embodiment, the antibody has the CDRs of antibody IMPI-039.

In one embodiment, the antibody has the CDRs of antibody IMPI-001.

In one embodiment, the antibody has the CDRs of antibody IMPI-019.

In one embodiment, the antibody has the CDRs of antibody IMPI-010.

In one embodiment, the antibody has the CDRs of antibody IMPI-008.

In one embodiment, the antibody has the CDRs of antibody IMPI-031.

In one embodiment, the antibody has the CDRs of antibody IMPI-057.

In one embodiment, the antibody has the CDRs of antibody IMPI-022.

In one embodiment, the antibody has the CDRs of antibody IMPI-035.

In one embodiment, the antibody has the CDRs of antibody IMPI-067.

In one embodiment, the antibody has the CDRs of antibody IMPI-072.

In one embodiment, the antibody comprises a variable heavy (VH) domain sequence and a variable light (VL) domain sequence and wherein the variable heavy (VH) domain and variable light (VL) domain sequences respectively comprise the variable heavy (VH) domain and variable light (VL) domain sequences of antibody IMPI-030, IMPI-053, IMPI-025, IMPI-040, IMPI-007, IMPI-020, IMPI-032, IMPI-023, IMPI-039, IMPI-001, IMPI-019, IMPI-010, IMPI-008, IMPI-031, IMPI-057, IMPI-022, IMPI-035, IMPI-067 or IMPI-072, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

In one embodiment, the antibody comprises a variable heavy (VH) domain sequence of antibody IMPI-030, optionally with 1, 2, 3, 4 or 5 amino acid alterations outside the complementarity determining regions (CDRs), and a variable light (VL) domain sequence of antibody IMPI-030, optionally with 1, 2, 3, 4 or 5 amino acid alterations outside the complementarity determining regions (CDRs).

In one embodiment, the antibody comprises a variable heavy (VH) domain sequence of antibody IMPI-053, optionally with 1, 2, 3, 4 or 5 amino acid alterations outside the complementarity determining regions (CDRs), and a variable light (VL) domain sequence of antibody IMPI-053, optionally with 1, 2, 3, 4 or 5 amino acid alterations outside the complementarity determining regions (CDRs).

In one embodiment, the antibody comprises a variable heavy (VH) domain sequence of antibody IMPI-025, optionally with 1, 2, 3, 4 or 5 amino acid alterations outside the complementarity determining regions (CDRs), and a variable light (VL) domain sequence of antibody IMPI-025, optionally with 1, 2, 3, 4 or 5 amino acid alterations outside the complementarity determining regions (CDRs).

In one embodiment, the antibody comprises a variable heavy (VH) domain sequence of antibody IMPI-040, optionally with 1, 2, 3, 4 or 5 amino acid alterations outside the complementarity determining regions (CDRs), and a variable light (VL) domain sequence of antibody IMPI-040, optionally with 1, 2, 3, 4 or 5 amino acid alterations outside the complementarity determining regions (CDRs).

In one embodiment, the antibody comprises a variable heavy (VH) domain sequence of antibody IMPI-007, optionally with 1, 2, 3, 4 or 5 amino acid alterations outside the complementarity determining regions (CDRs), and a variable light (VL) domain sequence of antibody IMPI-007, optionally with 1, 2, 3, 4 or 5 amino acid alterations outside the complementarity determining regions (CDRs).

In one embodiment, the antibody comprises a variable heavy (VH) domain sequence of antibody IMPI-020, optionally with 1, 2, 3, 4 or 5 amino acid alterations outside the complementarity determining regions (CDRs), and a variable light (VL) domain sequence of antibody IMPI-020, optionally with 1, 2, 3, 4 or 5 amino acid alterations outside the complementarity determining regions (CDRs).

In one embodiment, the antibody comprises a variable heavy (VH) domain sequence of antibody IMPI-032, optionally with 1, 2, 3, 4 or 5 amino acid alterations outside the complementarity determining regions (CDRs), and a variable light (VL) domain sequence of antibody IMPI-032, optionally with 1, 2, 3, 4 or 5 amino acid alterations outside the complementarity determining regions (CDRs).

In one embodiment, the antibody comprises a variable heavy (VH) domain sequence of antibody IMPI-023, optionally with 1, 2, 3, 4 or 5 amino acid alterations outside the complementarity determining regions (CDRs), and a variable light (VL) domain sequence of antibody IMPI-023, optionally with 1, 2, 3, 4 or 5 amino acid alterations outside the complementarity determining regions (CDRs).

In one embodiment, the antibody comprises a variable heavy (VH) domain sequence of antibody IMPI-039, optionally with 1, 2, 3, 4 or 5 amino acid alterations outside the complementarity determining regions (CDRs), and a variable light (VL) domain sequence of antibody IMPI-039, optionally with 1, 2, 3, 4 or 5 amino acid alterations outside the complementarity determining regions (CDRs).

In one embodiment, the antibody comprises a variable heavy (VH) domain sequence of antibody IMPI-001, optionally with 1, 2, 3, 4 or 5 amino acid alterations outside the complementarity determining regions (CDRs), and a variable light (VL) domain sequence of antibody IMPI-001, optionally with 1, 2, 3, 4 or 5 amino acid alterations outside the complementarity determining regions (CDRs).

In one embodiment, the antibody comprises a variable heavy (VH) domain sequence of antibody IMPI-019, optionally with 1, 2, 3, 4 or 5 amino acid alterations outside the complementarity determining regions (CDRs), and a variable light (VL) domain sequence of antibody IMPI-019, optionally with 1, 2, 3, 4 or 5 amino acid alterations outside the complementarity determining regions (CDRs).

In one embodiment, the antibody comprises a variable heavy (VH) domain sequence of antibody IMPI-010, optionally with 1, 2, 3, 4 or 5 amino acid alterations outside the complementarity determining regions (CDRs), and a variable light (VL) domain sequence of antibody IMPI-010, optionally with 1, 2, 3, 4 or 5 amino acid alterations outside the complementarity determining regions (CDRs).

In one embodiment, the antibody comprises a variable heavy (VH) domain sequence of antibody IMPI-008, optionally with 1, 2, 3, 4 or 5 amino acid alterations outside the complementarity determining regions (CDRs), and a variable light (VL) domain sequence of antibody IMPI-008, optionally with 1, 2, 3, 4 or 5 amino acid alterations outside the complementarity determining regions (CDRs).

In one embodiment, the antibody comprises a variable heavy (VH) domain sequence of antibody IMPI-031, optionally with 1, 2, 3, 4 or 5 amino acid alterations outside the complementarity determining regions (CDRs), and a variable light (VL) domain sequence of antibody IMPI-031, optionally with 1, 2, 3, 4 or 5 amino acid alterations outside the complementarity determining regions (CDRs).

In one embodiment, the antibody comprises a variable heavy (VH) domain sequence of antibody IMPI-057, optionally with 1, 2, 3, 4 or 5 amino acid alterations outside the complementarity determining regions (CDRs), and a variable light (VL) domain sequence of antibody IMPI-057, optionally with 1, 2, 3, 4 or 5 amino acid alterations outside the complementarity determining regions (CDRs).

In one embodiment, the antibody comprises a variable heavy (VH) domain sequence of antibody IMPI-022, optionally with 1, 2, 3, 4 or 5 amino acid alterations outside the complementarity determining regions (CDRs), and a variable light (VL) domain sequence of antibody IMPI-022, optionally with 1, 2, 3, 4 or 5 amino acid alterations outside the complementarity determining regions (CDRs).

In one embodiment, the antibody comprises a variable heavy (VH) domain sequence of antibody IMPI-035, optionally with 1, 2, 3, 4 or 5 amino acid alterations outside the complementarity determining regions (CDRs), and a variable light (VL) domain sequence of antibody IMPI-035, optionally with 1, 2, 3, 4 or 5 amino acid alterations outside the complementarity determining regions (CDRs).

In one embodiment, the antibody comprises a variable heavy (VH) domain sequence of antibody IMPI-067, optionally with 1, 2, 3, 4 or 5 amino acid alterations outside the complementarity determining regions (CDRs), and a variable light (VL) domain sequence of antibody IMPI-067, optionally with 1, 2, 3, 4 or 5 amino acid alterations outside the complementarity determining regions (CDRs).

In one embodiment, the antibody comprises a variable heavy (VH) domain sequence of antibody IMPI-072, optionally with 1, 2, 3, 4 or 5 amino acid alterations outside the complementarity determining regions (CDRs), and a variable light (VL) domain sequence of antibody IMPI-072, optionally with 1, 2, 3, 4 or 5 amino acid alterations outside the complementarity determining regions (CDRs).

In one embodiment, the antibody comprises a variable heavy (VH) domain sequence and a variable light (VL) domain sequence and wherein the variable heavy (VH) domain and variable light (VL) domain sequences respectively comprise a sequence having at least 90% identity to the variable heavy (VH) and variable light (VL) domain sequences of antibody IMPI-030, IMPI-053, IMPI-025, IMPI-040, IMPI-007, IMPI-020, IMPI-032, IMPI-023, IMPI-039, IMPI-001, IMPI-019, IMPI-010, IMPI-008, IMPI-031, IMPI-057, IMPI-022, IMPI-035, IMPI-067 or IMPI-072, provided that the antibody has the CDRs of antibody IMPI-030, IMPI-053, IMPI-025, IMPI-040, IMPI-007, IMPI-020, IMPI-032, IMPI-023, IMPI-039, IMPI-001, IMPI-019, IMPI-010, IMPI-008, IMPI-031, IMPI-057, IMPI-022, IMPI-035, IMPI-067 or IMPI-072.

In one embodiment, the variable heavy (VH) domain sequence comprises a sequence having at least 90% identity to the VH domain sequence of antibody IMPI-030 and the variable light (VL) domain sequence comprises a sequence having at least 90% identity to the VL domain sequence of antibody IMPI-030, provided that the antibody has the CDRs of antibody IMPI-030.

In one embodiment, variable heavy (VH) domain sequence comprises a sequence having at least 90% identity to the VH domain sequence of antibody IMPI-053 and the variable light (VL) domain sequence comprises a sequence having at least 90% identity to the VL domain sequence of antibody IMPI-053, provided that the antibody has the CDRs of antibody IMPI-053.

In one embodiment, the variable heavy (VH) domain sequence comprises a sequence having at least 90% identity to the VH domain sequence of antibody IMPI-025 and the variable light (VL) domain sequence comprises a sequence having at least 90% identity to the VL domain sequence of antibody IMPI-025, provided that the antibody has the CDRs of antibody IMPI-025.

In one embodiment, the variable heavy (VH) domain sequence comprises a sequence having at least 90% identity to the VH domain sequence of antibody IMPI-040 and the variable light (VL) domain sequence comprises a sequence having at least 90% identity to the VL domain sequence of antibody IMPI-040, provided that the antibody has the CDRs of antibody IMPI-040.

In one embodiment, the variable heavy (VH) domain sequence comprises a sequence having at least 90% identity to the VH domain sequence of antibody IMPI-007 and the variable light (VL) domain sequence comprises a sequence having at least 90% identity to the VL domain sequence of antibody IMPI-007, provided that the antibody has the CDRs of antibody IMPI-007.

In one embodiment, the variable heavy (VH) domain sequence comprises a sequence having at least 90% identity to the VH domain sequence of antibody IMPI-020 and the variable light (VL) domain sequence comprises a sequence having at least 90% identity to the VL domain sequence of antibody IMPI-020, provided that the antibody has the CDRs of antibody IMPI-020.

In one embodiment, the variable heavy (VH) domain sequence comprises a sequence having at least 90% identity to the VH domain sequence of antibody IMPI-032 and the variable light (VL) domain sequence comprises a sequence having at least 90% identity to the VL domain sequence of antibody IMPI-032, provided that the antibody has the CDRs of antibody IMPI-032.

In one embodiment, the variable heavy (VH) domain sequence comprises a sequence having at least 90% identity to the VH domain sequence of antibody IMPI-023 and the variable light (VL) domain sequence comprises a sequence having at least 90% identity to the VL domain sequence of antibody IMPI-023, provided that the antibody has the CDRs of antibody IMPI-023.

In one embodiment, the variable heavy (VH) domain sequence comprises a sequence having at least 90% identity to the VH domain sequence of antibody IMPI-039 and the variable light (VL) domain sequence comprises a sequence having at least 90% identity to the VL domain sequence of antibody IMPI-039, provided that the antibody has the CDRs of antibody IMPI-039.

In one embodiment, the variable heavy (VH) domain sequence comprises a sequence having at least 90% identity to the VH domain sequence of antibody IMPI-001 and the variable light (VL) domain sequence comprises a sequence having at least 90% identity to the VL domain sequence of antibody IMPI-001, provided that the antibody has the CDRs of antibody IMPI-001.

In one embodiment, the variable heavy (VH) domain sequence comprises a sequence having at least 90% identity to the VH domain sequence of antibody IMPI-019 and the variable light (VL) domain sequence comprises a sequence having at least 90% identity to the VL domain sequence of antibody IMPI-019, provided that the antibody has the CDRs of antibody IMPI-019.

In one embodiment, the variable heavy (VH) domain sequence comprises a sequence having at least 90% identity to the VH domain sequence of antibody IMPI-010 and the variable light (VL) domain sequence comprises a sequence having at least 90% identity to the VL domain sequence of antibody IMPI-010, provided that the antibody has the CDRs of antibody IMPI-010.

In one embodiment, the variable heavy (VH) domain sequence comprises a sequence having at least 90% identity to the VH domain sequence of antibody IMPI-008 and the variable light (VL) domain sequence comprises a sequence having at least 90% identity to the VL domain sequence of antibody IMPI-008, provided that the antibody has the CDRs of antibody IMPI-008.

In one embodiment, the variable heavy (VH) domain sequence comprises a sequence having at least 90% identity to the VH domain sequence of antibody IMPI-031 and the variable light (VL) domain sequence comprises a sequence having at least 90% identity to the VL domain sequence of antibody IMPI-031, provided that the antibody has the CDRs of antibody IMPI-031.

In one embodiment, the variable heavy (VH) domain sequence comprises a sequence having at least 90% identity to the VH domain sequence of antibody IMPI-057 and the variable light (VL) domain sequence comprises a sequence having at least 90% identity to the VL domain sequence of antibody IMPI-057, provided that the antibody has the CDRs of antibody IMPI-057.

In one embodiment, the variable heavy (VH) domain sequence comprises a sequence having at least 90% identity to the VH domain sequence of antibody IMPI-022 and the variable light (VL) domain sequence comprises a sequence having at least 90% identity to the VL domain sequence of antibody IMPI-022, provided that the antibody has the CDRs of antibody IMPI-022.

In one embodiment, the variable heavy (VH) domain sequence comprises a sequence having at least 90% identity to the VH domain sequence of antibody IMPI-035 and the variable light (VL) domain sequence comprises a sequence having at least 90% identity to the VL domain sequence of antibody IMPI-035, provided that the antibody has the CDRs of antibody IMPI-035.

In one embodiment, the variable heavy (VH) domain sequence comprises a sequence having at least 90% identity to the VH domain sequence of antibody IMPI-067 and the variable light (VL) domain sequence comprises a sequence having at least 90% identity to the VL domain sequence of antibody IMPI-067, provided that the antibody has the CDRs of antibody IMPI-067.

In one embodiment, the variable heavy (VH) domain sequence comprises a sequence having at least 90% identity to the VH domain sequence of antibody IMPI-072 and the variable light (VL) domain sequence comprises a sequence having at least 90% identity to the VL domain sequence of antibody IMPI-072, provided that the antibody has the CDRs of antibody IMPI-072.

In one embodiment, the antibody comprises a variable heavy (VH) domain sequence and a variable light (VL) domain sequence and wherein the variable heavy (VH) domain and variable light (VL) domain sequences respectively comprise the variable heavy (VH) and variable light (VL) domain sequences of antibody IMPI-030, IMPI-053, IMPI-025, IMPI-040, IMPI-007, IMPI-020, IMPI-032, IMPI-023, IMPI-039, IMPI-001, IMPI-019, IMPI-010, IMPI-008, IMPI-031, IMPI-057, IMPI-022, IMPI-035, IMPI-067 or IMPI-072.

In one embodiment, the present invention provides an anti-SARS-CoV-2 antibody, wherein the antibody comprises a variable heavy (VH) domain sequence and a variable light (VL) domain sequence and wherein the variable heavy (VH) domain and variable light (VL) domain sequences respectively comprise the variable heavy (VH) and variable light (VL) domain sequences of antibody IMPI-030, IMPI-053, IMPI-025, IMPI-040, IMPI-007, IMPI-020, IMPI-032, IMPI-023, IMPI-039, IMPI-001, IMPI-019, IMPI-010, IMPI-008, IMPI-031, IMPI-057, IMPI-022, IMPI-035, IMPI-067 or IMPI-072.

In one embodiment, the variable heavy (VH) domain sequence comprises the VH domain sequence of antibody IMPI-030 and the variable light (VL) domain sequence comprises the VL domain sequence of antibody IMPI-030.

In one embodiment, the variable heavy (VH) domain sequence comprises the VH domain sequence of antibody IMPI-053 and the variable light (VL) domain sequence comprises the VL domain sequence of antibody IMPI-053.

In one embodiment, the variable heavy (VH) domain sequence comprises the VH domain sequence of antibody IMPI-025 and the variable light (VL) domain sequence comprises the VL domain sequence of antibody IMPI-025.

In one embodiment, the variable heavy (VH) domain sequence comprises the VH domain sequence of antibody IMPI-040 and the variable light (VL) domain sequence comprises the VL domain sequence of antibody IMPI-040.

In one embodiment, the variable heavy (VH) domain sequence comprises the VH domain sequence of antibody IMPI-007 and the variable light (VL) domain sequence comprises the VL domain sequence of antibody IMPI-007.

In one embodiment, the variable heavy (VH) domain sequence comprises the VH domain sequence of antibody IMPI-020 and the variable light (VL) domain sequence comprises the VL domain sequence of antibody IMPI-020.

In one embodiment, the variable heavy (VH) domain sequence comprises the VH domain sequence of antibody IMPI-032 and the variable light (VL) domain sequence comprises the VL domain sequence of antibody IMPI-032.

In one embodiment, the variable heavy (VH) domain sequence comprises the VH domain sequence of antibody IMPI-023 and the variable light (VL) domain sequence comprises the VL domain sequence of antibody IMPI-023.

In one embodiment, the variable heavy (VH) domain sequence comprises the VH domain sequence of antibody IMPI-039 and the variable light (VL) domain sequence comprises the VL domain sequence of antibody IMPI-039.

In one embodiment, the variable heavy (VH) domain sequence comprises the VH domain sequence of antibody IMPI-001 and the variable light (VL) domain sequence comprises the VL domain sequence of antibody IMPI-001.

In one embodiment, the variable heavy (VH) domain sequence comprises the VH domain sequence of antibody IMPI-019 and the variable light (VL) domain sequence comprises the VL domain sequence of antibody IMPI-019.

In one embodiment, the variable heavy (VH) domain sequence comprises the VH domain sequence of antibody IMPI-010 and the variable light (VL) domain sequence comprises the VL domain sequence of antibody IMPI-010.

In one embodiment, the variable heavy (VH) domain sequence comprises the VH domain sequence of antibody IMPI-008 and the variable light (VL) domain sequence comprises the VL domain sequence of antibody IMPI-008.

In one embodiment, the variable heavy (VH) domain sequence comprises the VH domain sequence of antibody IMPI-031 and the variable light (VL) domain sequence comprises the VL domain sequence of antibody IMPI-031.

In one embodiment, the variable heavy (VH) domain sequence comprises the VH domain sequence of antibody IMPI-057 and the variable light (VL) domain sequence comprises the VL domain sequence of antibody IMPI-057.

In one embodiment, the variable heavy (VH) domain sequence comprises the VH domain sequence of antibody IMPI-022 and the variable light (VL) domain sequence comprises the VL domain sequence of antibody IMPI-022.

In one embodiment, the variable heavy (VH) domain sequence comprises the VH domain sequence of antibody IMPI-035 and the variable light (VL) domain sequence comprises the VL domain sequence of antibody IMPI-035.

In one embodiment, the variable heavy (VH) domain sequence comprises the VH domain sequence of antibody IMPI-067 and the variable light (VL) domain sequence comprises the VL domain sequence of antibody IMPI-067.

In one embodiment, the variable heavy (VH) domain sequence comprises the VH domain sequence of antibody IMPI-072 and the variable light (VL) domain sequence comprises the VL domain sequence of antibody IMPI-072.

In one embodiment, the antibody comprises VH and/or VL domain framework regions of human germline gene segment sequences.

In one embodiment, the antibody comprises an antibody VH domain which
i) is derived from recombination of a human heavy chain V gene segment, a human heavy chain D gene segment and a human heavy chain J gene segment, wherein
   the V gene segment is IGHV4-4*02, IGHV3-9*01 or IGHV3-30*18;
   and/or
   the J gene segment is IGHJ4*02 or IGHJ6*02, or
ii) comprises framework regions FR1, FR2, FR3 and FR4, wherein
   FR1 aligns with human germline V gene segment IGHV4-4*02, IGHV3-9*01 or IGHV3-30*18 with up to 1, 2, 3, 4, or 5 amino acid alterations,
   FR2 aligns with human germline V gene segment IGHV4-4*02, IGHV3-9*01 or IGHV3-30*18 with up to 1, 2, 3, 4, or 5 amino acid alterations,
   FR3 aligns with human germline V gene segment IGHV4-4*02, IGHV3-9*01 or IGHV3-30*18 with up to 1, 2, 3, 4, or 5 amino acid alterations, and/or
   FR4 aligns with human germline J gene segment IGHJ4*02 or IGHJ6*02 with up to 1, 2, 3, 4 or 5 amino acid alterations.

In one embodiment, the antibody comprises an antibody VH domain which
i) is derived from recombination of a human heavy chain V gene segment IGHV4-4*02, IGHV3-9*01 or IGHV3-30*18, a human heavy chain D gene segment and a human heavy chain J gene segment, or
ii) comprises framework regions FR1, FR2, FR3 and FR4, wherein FR1, FR2 and FR3 each align with human germline V segment IGHV4-4*02, IGHV3-9*01 or IGHV3-30*18 with up to 1, 2, 3, 4 or 5 amino acid alterations.

In one embodiment, the J gene segment is IGHJ4*02 or IGHJ6*02, or the VH domain framework region FR4 aligns with human germline J gene segment IGHJ4*02 or IGHJ6*02 with 1, 2, 3, 4 or 5 amino acid alterations.

In one embodiment, the antibody comprises an antibody VL domain which
i) is derived from recombination of a human light chain V gene segment and a human light chain J gene segment, wherein
   the V gene segment is IGKV2D-30*01, IGKV1D-13*d01 or IGKV3-20*01, and/or
   the J gene segment is IGKJ4*01 or
ii) comprises framework regions FR1, FR2, FR3 and FR4, wherein
   FR1 aligns with human germline V gene segment IGKV2D-30*01, IGKV1D-13*d01 or IGKV3-20*01 with up to 1, 2, 3, 4, or 5 amino acid alterations,
   FR2 aligns with human germline V gene segment IGKV2D-30*01, IGKV1D-13*d01 or IGKV3-20*01 with up to 1, 2, 3, 4, or 5 amino acid alterations
   FR3 aligns with human germline V gene segment IGKV2D-30*01, IGKV1D-13*d01 or IGKV3-20*01 with up to 1, 2, 3, 4 or 5 amino acid alterations, and/or
   FR4 aligns with human germline J gene segment IGKJ4*01 with up to 1, 2, 3, 4 or 5 amino acid alterations.

In one embodiment, the antibody comprises an antibody VL domain derived from recombination of a human light chain V gene segment and a human light chain J gene segment, wherein:
the V gene segment is IGKV2D-30*01, IGKV1D-13*d01 or IGKV3-20*01, and optionally the J gene segment is IGKJ4*01.

Example combinations of v and j gene segments for heavy and light chain variable domains are shown in Table 3, and these represent preferred combinations. The heavy and light chain variable domains may optionally be derived from the v and j gene segments identified in Table 3 for any one individual IMPI antibody identified in this Group B section.

### Group C—S2 Binders:

In a third aspect, the present invention provides a neutralising antibody that specifically binds to the S2 subunit of the SARS-CoV-2 spike protein.

In one embodiment, the antibody neutralises SARS-CoV-2 with an IC50 of 10 nM or lower (e.g. as measured in a pseudovirus neutralisation assay).

In one embodiment, the antibody neutralises SARS-CoV-2 with an IC50 of 5 nM or lower (e.g. as measured in a pseudovirus neutralisation assay).

In one embodiment, the antibody neutralises SARS-CoV-2 with an IC50 of 3 nM or lower (e.g. as measured in a pseudovirus neutralisation assay).

In one embodiment, the antibody comprises a variable heavy (VH) domain sequence comprising complementarity determining regions (CDRs) HCDR1, HCDR2 and HCDR3, and a variable light (VL) domain sequence comprising complementarity determining regions LCDR1, LCDR2 and LCDR3, wherein the HCDR3 is the HCDR3 of antibody IMPI-003, IMPI-013, IMPI-063, IMPI-061, IMPI-062, IMPI-064, IMPI-065, IMPI-066, IMPI-069, IMPI-070 or IMPI-071.

In one embodiment, the HCDR3 is the HCDR3 of antibody IMPI-003.

In one embodiment, the HCDR3 is the HCDR3 of antibody IMPI-013.

In one embodiment, the HCDR3 is the HCDR3 of antibody IMPI-063.

In one embodiment, the HCDR3 is the HCDR3 of antibody IMPI-061.

In one embodiment, the HCDR3 is the HCDR3 of antibody IMPI-062.

In one embodiment, the HCDR3 is the HCDR3 of antibody IMPI-064.

In one embodiment, the HCDR3 is the HCDR3 of antibody IMPI-065.

In one embodiment, the HCDR3 is the HCDR3 of antibody IMPI-066.

In one embodiment, the HCDR3 is the HCDR3 of antibody IMPI-069.

In one embodiment, the HCDR3 is the HCDR3 of antibody IMPI-070.

In one embodiment, the HCDR3 is the HCDR3 of antibody IMPI-071.

In one embodiment, the present invention provides an anti-SARS-CoV-2 antibody,

wherein the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3,
wherein the CDRs are those of antibody IMPI-003, IMPI-013, IMPI-063, IMPI-061, IMPI-062, IMPI-064, IMPI-065, IMPI-066, IMPI-069, IMPI-070 or IMPI-071.

In one embodiment, the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3, wherein the CDRs are those of antibody IMPI-003, IMPI-013, IMPI-063, IMPI-061, IMPI-062, IMPI-064, IMPI-065, IMPI-066, IMPI-069, IMPI-070 or IMPI-071.

In one embodiment, the antibody has the CDRs of antibody IMPI-003.

In one embodiment, the antibody has the CDRs of antibody IMPI-013.

In one embodiment, the antibody has the CDRs of antibody IMPI-063.

In one embodiment, the antibody has the CDRs of antibody IMPI-061.

In one embodiment, the antibody has the CDRs of antibody IMPI-062.

In one embodiment, the antibody has the CDRs of antibody IMPI-064.

In one embodiment, the antibody has the CDRs of antibody IMPI-065.

In one embodiment, the antibody has the CDRs of antibody IMPI-066.

In one embodiment, the antibody has the CDRs of antibody IMPI-069.

In one embodiment, the antibody has the CDRs of antibody IMPI-070.

In one embodiment, the antibody has the CDRs of antibody IMPI-071.

In one embodiment, the antibody comprises a variable heavy (VH) domain sequence and a variable light (VL) domain sequence and wherein the variable heavy (VH) domain and variable light (VL) domain sequences respectively comprise the variable heavy (VH) domain and variable light (VL) domain sequences of antibody IMPI-003, IMPI-013, IMPI-063, IMPI-061, IMPI-062, IMPI-064, IMPI-065, IMPI-066, IMPI-069, IMPI-070 or IMPI-071, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

In one embodiment, wherein the antibody comprises a variable heavy (VH) domain sequence of antibody IMPI-003, optionally with 1, 2, 3, 4 or 5 amino acid alterations outside the complementarity determining regions (CDRs), and a variable light (VL) domain sequence of antibody IMPI-003, optionally with 1, 2, 3, 4 or 5 amino acid alterations outside the complementarity determining regions (CDRs).

In one embodiment, the antibody comprises a variable heavy (VH) domain sequence of antibody IMPI-013, optionally with 1, 2, 3, 4 or 5 amino acid alterations outside the complementarity determining regions (CDRs), and a variable light (VL) domain sequence of antibody IMPI-013, optionally with 1, 2, 3, 4 or 5 amino acid alterations outside the complementarity determining regions (CDRs).

In one embodiment, the antibody comprises a variable heavy (VH) domain sequence of antibody IMPI-063, optionally with 1, 2, 3, 4 or 5 amino acid alterations outside the complementarity determining regions (CDRs), and a variable light (VL) domain sequence of antibody IMPI-063, optionally with 1, 2, 3, 4 or 5 amino acid alterations outside the complementarity determining regions (CDRs).

In one embodiment, the antibody comprises a variable heavy (VH) domain sequence of antibody IMPI-061, optionally with 1, 2, 3, 4 or 5 amino acid alterations outside the complementarity determining regions (CDRs), and a variable light (VL) domain sequence of antibody IMPI-061, optionally with 1, 2, 3, 4 or 5 amino acid alterations outside the complementarity determining regions (CDRs).

In one embodiment, the antibody comprises a variable heavy (VH) domain sequence of antibody IMPI-062, optionally with 1, 2, 3, 4 or 5 amino acid alterations outside the complementarity determining regions (CDRs), and a variable light (VL) domain sequence of antibody IMPI-062, optionally with 1, 2, 3, 4 or 5 amino acid alterations outside the complementarity determining regions (CDRs).

In one embodiment, the antibody comprises a variable heavy (VH) domain sequence of antibody IMPI-064, optionally with 1, 2, 3, 4 or 5 amino acid alterations outside the complementarity determining regions (CDRs), and a variable light (VL) domain sequence of antibody IMPI-064, optionally with 1, 2, 3, 4 or 5 amino acid alterations outside the complementarity determining regions (CDRs).

In one embodiment, the antibody comprises a variable heavy (VH) domain sequence of antibody IMPI-065, optionally with 1, 2, 3, 4 or 5 amino acid alterations outside the complementarity determining regions (CDRs), and a variable light (VL) domain sequence of antibody IMPI-065, optionally with 1, 2, 3, 4 or 5 amino acid alterations outside the complementarity determining regions (CDRs).

In one embodiment, the antibody comprises a variable heavy (VH) domain sequence of antibody IMPI-066, optionally with 1, 2, 3, 4 or 5 amino acid alterations outside the complementarity determining regions (CDRs), and a variable light (VL) domain sequence of antibody IMPI-066, optionally with 1, 2, 3, 4 or 5 amino acid alterations outside the complementarity determining regions (CDRs).

In one embodiment, the antibody comprises a variable heavy (VH) domain sequence of antibody IMPI-069, optionally with 1, 2, 3, 4 or 5 amino acid alterations outside the complementarity determining regions (CDRs), and a variable light (VL) domain sequence of antibody IMPI-069, optionally with 1, 2, 3, 4 or 5 amino acid alterations outside the complementarity determining regions (CDRs).

In one embodiment, the antibody comprises a variable heavy (VH) domain sequence of antibody IMPI-070, optionally with 1, 2, 3, 4 or 5 amino acid alterations outside the complementarity determining regions (CDRs), and a variable light (VL) domain sequence of antibody IMPI-070, optionally with 1, 2, 3, 4 or 5 amino acid alterations outside the complementarity determining regions (CDRs).

In one embodiment, the antibody comprises a variable heavy (VH) domain sequence of antibody IMPI-071, optionally with 1, 2, 3, 4 or 5 amino acid alterations outside the complementarity determining regions (CDRs), and a variable light (VL) domain sequence of antibody IMPI-071, optionally with 1, 2, 3, 4 or 5 amino acid alterations outside the complementarity determining regions (CDRs).

In one embodiment, the antibody comprises a variable heavy (VH) domain sequence and a variable light (VL) domain sequence and wherein the variable heavy (VH) domain and variable light (VL) domain sequences respectively comprise a sequence having at least 90% identity to the variable heavy (VH) and variable light (VL) domain sequences of antibody IMPI-003, IMPI-013, IMPI-063, IMPI-061, IMPI-062, IMPI-064, IMPI-065, IMPI-066, IMPI-069, IMPI-070 or IMPI-071, provided that the antibody has the CDRs of antibody IMPI-003, IMPI-013, IMPI-063, IMPI-061, IMPI-062, IMPI-064, IMPI-065, IMPI-066, IMPI-069, IMPI-070 or IMPI-071.

In one embodiment, the variable heavy (VH) domain sequence comprises a sequence having at least 90% identity to the VH domain sequence of antibody IMPI-003 and the variable light (VL) domain sequence comprises a sequence having at least 90% identity to the VL domain sequence of antibody IMPI-003, provided that the antibody has the CDRs of antibody IMPI-003.

In one embodiment, the variable heavy (VH) domain sequence comprises a sequence having at least 90% identity to the VH domain sequence of antibody IMPI-013 and the variable light (VL) domain sequence comprises a sequence having at least 90% identity to the VL domain sequence of antibody IMPI-013, provided that the antibody has the CDRs of antibody IMPI-013.

In one embodiment, the variable heavy (VH) domain sequence comprises a sequence having at least 90% identity to the VH domain sequence of antibody IMPI-063 and the variable light (VL) domain sequence comprises a sequence having at least 90% identity to the VL domain sequence of antibody IMPI-063, provided that the antibody has the CDRs of antibody IMPI-063.

In one embodiment, the variable heavy (VH) domain sequence comprises a sequence having at least 90% identity to the VH domain sequence of antibody IMPI-061 and the variable light (VL) domain sequence comprises a sequence having at least 90% identity to the VL domain sequence of antibody IMPI-061, provided that the antibody has the CDRs of antibody IMPI-061.

In one embodiment, the variable heavy (VH) domain sequence comprises a sequence having at least 90% identity to the VH domain sequence of antibody IMPI-062 and the variable light (VL) domain sequence comprises a sequence having at least 90% identity to the VL domain sequence of antibody IMPI-062, provided that the antibody has the CDRs of antibody IMPI-062.

In one embodiment, the variable heavy (VH) domain sequence comprises a sequence having at least 90% identity to the VH domain sequence of antibody IMPI-064 and the variable light (VL) domain sequence comprises a sequence having at least 90% identity to the VL domain sequence of antibody IMPI-064, provided that the antibody has the CDRs of antibody IMPI-064.

In one embodiment, the variable heavy (VH) domain sequence comprises a sequence having at least 90% identity to the VH domain sequence of antibody IMPI-065 and the variable light (VL) domain sequence comprises a sequence having at least 90% identity to the VL domain sequence of antibody IMPI-065, provided that the antibody has the CDRs of antibody IMPI-065.

In one embodiment, the variable heavy (VH) domain sequence comprises a sequence having at least 90% identity to the VH domain sequence of antibody IMPI-066 and the variable light (VL) domain sequence comprises a sequence having at least 90% identity to the VL domain sequence of antibody IMPI-066, provided that the antibody has the CDRs of antibody IMPI-066.

In one embodiment, the variable heavy (VH) domain sequence comprises a sequence having at least 90% identity to the VH domain sequence of antibody IMPI-069 and the variable light (VL) domain sequence comprises a sequence having at least 90% identity to the VL domain sequence of antibody IMPI-069, provided that the antibody has the CDRs of antibody IMPI-069.

In one embodiment, the variable heavy (VH) domain sequence comprises a sequence having at least 90% identity to the VH domain sequence of antibody IMPI-070 and the variable light (VL) domain sequence comprises a sequence having at least 90% identity to the VL domain sequence of antibody IMPI-070, provided that the antibody has the CDRs of antibody IMPI-070.

In one embodiment, the variable heavy (VH) domain sequence comprises a sequence having at least 90% identity to the VH domain sequence of antibody IMPI-071 and the variable light (VL) domain sequence comprises a sequence having at least 90% identity to the VL domain sequence of antibody IMPI-071, provided that the antibody has the CDRs of antibody IMPI-071.

In one embodiment, the antibody comprises a variable heavy (VH) domain sequence and a variable light (VL) domain sequence and wherein the variable heavy (VH) domain and variable light (VL) domain sequences respectively comprise the variable heavy (VH) and variable light (VL) domain sequences of antibody IMPI-003, IMPI-013, IMPI-063, IMPI-061, IMPI-062, IMPI-064, IMPI-065, IMPI-066, IMPI-069, IMPI-070 or IMPI-071.

In one embodiment, the present invention provides an anti-SARS-CoV-2 antibody, wherein the antibody comprises a variable heavy (VH) domain sequence and a variable light (VL) domain sequence and wherein the variable heavy (VH) domain and variable light (VL) domain sequences respectively comprise the variable heavy (VH) and variable light (VL) domain sequences of antibody IMPI-003, IMPI-013, IMPI-063, IMPI-061, IMPI-062, IMPI-064, IMPI-065, IMPI-066, IMPI-069, IMPI-070 or IMPI-071.

In one embodiment, the variable heavy (VH) domain sequence comprises the VH domain sequence of antibody IMPI-003 and the variable light (VL) domain sequence comprises the VL domain sequence of antibody IMPI-003.

In one embodiment, the variable heavy (VH) domain sequence comprises the VH domain sequence of antibody IMPI-013 and the variable light (VL) domain sequence comprises the VL domain sequence of antibody IMPI-013.

In one embodiment, the variable heavy (VH) domain sequence comprises the VH domain sequence of antibody IMPI-063 and the variable light (VL) domain sequence comprises the VL domain sequence of antibody IMPI-063.

In one embodiment, the variable heavy (VH) domain sequence comprises the VH domain sequence of antibody IMPI-061 and the variable light (VL) domain sequence comprises the VL domain sequence of antibody IMPI-061.

In one embodiment, the variable heavy (VH) domain sequence comprises the VH domain sequence of antibody IMPI-062 and the variable light (VL) domain sequence comprises the VL domain sequence of antibody IMPI-062.

In one embodiment, the variable heavy (VH) domain sequence comprises the VH domain sequence of antibody IMPI-064 and the variable light (VL) domain sequence comprises the VL domain sequence of antibody IMPI-064.

In one embodiment, the variable heavy (VH) domain sequence comprises the VH domain sequence of antibody IMPI-065 and the variable light (VL) domain sequence comprises the VL domain sequence of antibody IMPI-065.

In one embodiment, the variable heavy (VH) domain sequence comprises the VH domain sequence of antibody IMPI-066 and the variable light (VL) domain sequence comprises the VL domain sequence of antibody IMPI-066.

In one embodiment, the variable heavy (VH) domain sequence comprises the VH domain sequence of antibody IMPI-069 and the variable light (VL) domain sequence comprises the VL domain sequence of antibody IMPI-069.

In one embodiment, the variable heavy (VH) domain sequence comprises the VH domain sequence of antibody IMPI-070 and the variable light (VL) domain sequence comprises the VL domain sequence of antibody IMPI-070.

In one embodiment, the variable heavy (VH) domain sequence comprises the VH domain sequence of antibody IMPI-071 and the variable light (VL) domain sequence comprises the VL domain sequence of antibody IMPI-071.

In one embodiment, the antibody comprises VH and/or VL domain framework regions of human germline gene segment sequences.

In one embodiment, the antibody comprises an antibody VH domain which
i) is derived from recombination of a human heavy chain V gene segment, a human heavy chain D gene segment and a human heavy chain J gene segment, wherein the V gene segment is IGHV3-9*01 or IGHV3-20*d01;
and/or the J gene segment is IGHJ6*02 or IGHJ4*02, or
ii) comprises framework regions FR1, FR2, FR3 and FR4, wherein FR1 aligns with human germline V gene segment IGHV3-9*01 or IGHV3-20*d01 with up to 1, 2, 3, 4, or 5 amino acid alterations, FR2 aligns with human germline V gene segment IGHV3-9*01 or IGHV3-20*d01 with up to 1, 2, 3, 4, or 5 amino acid alterations, FR3 aligns with human germline V gene segment IGHV3-9*01 or IGHV3-20*d01 with up to 1, 2, 3, 4, or 5 amino acid alterations, and/or FR4 aligns with human germline J gene segment IGHJ6*02 or IGHJ4*02 with up to 1, 2, 3, 4 or 5 amino acid alterations.

In one embodiment, the antibody comprises an antibody VH domain which
i) is derived from recombination of a human heavy chain V gene segment IGHV3-9*01 or IGHV3-20*d01, a human heavy chain D gene segment and a human heavy chain J gene segment, or
ii) comprises framework regions FR1, FR2, FR3 and FR4, wherein FR1, FR2 and FR3 each align with human germline V segment IGHV3-9*01 or IGHV3-20*d01 with up to 1, 2, 3, 4 or 5 amino acid alterations.

In one embodiment, the J gene segment is IGHJ6*02 or IGHJ4*02, or the VH domain framework region FR4 aligns with human germline J gene segment IGHJ6*02 or IGHJ4*02 with 1, 2, 3, 4 or 5 amino acid alterations.

In one embodiment, the antibody comprises an antibody VL domain which
i) is derived from recombination of a human light chain V gene segment and a human light chain J gene segment, wherein the V gene segment is IGKV1-6*01 or IGKV3-20*01, and/or the J gene segment is IGKJ1*01 or IGKJ2*04; or
ii) comprises framework regions FR1, FR2, FR3 and FR4, wherein FR1 aligns with human germline V gene segment IGKV1-6*01 or IGKV3-20*01 with up to 1, 2, 3, 4, or 5 amino acid alterations, FR2 aligns with human germline V gene segment IGKV1-6*01 or IGKV3-20*01 with up to 1, 2, 3, 4, or 5 amino acid alterations FR3 aligns with human germline V gene segment IGKV1-6*01 or IGKV3-20*01 with up to 1, 2, 3, 4 or 5 amino acid alterations, and/or FR4 aligns with human germline J gene segment IGKJ1*01 or IGKJ2*04 with up to 1, 2, 3, 4 or 5 amino acid alterations.

In one embodiment, the antibody comprises an antibody VL domain derived from recombination of a human light chain V gene segment and a human light chain J gene segment, wherein: the V gene segment is IGKV1-6*01 or IGKV3-20*01, and optionally the J gene segment is IGKJ1*01 or IGKJ2*04.

Example combinations of v and j gene segments for heavy and light chain variable domains are shown in Table 3, and these represent preferred combinations. The heavy and light chain variable domains may optionally be derived from the v and j gene segments identified in Table 3 for any one individual IMPI antibody identified in this Group C section.

Further S2 binders According to the third aspect of the invention, further antibodies are provided herein which specifically bind to the S2 subunit of the SARS-CoV-2 spike protein. For example, antibodies YANG-1201, YANG-1202, YANG-1203, YANG-1204, YANG-1205, YANG-1206, YANG-1207, YANG-2201, YANG-2202, YANG-2203, YANG-2203a, YANG-2203b, YANG-2203c, YANG-2203d, YANG-2203e, YANG-2203f, YANG-2203g, YANG-2203h, YANG-2203i, YANG-2203j, YANG-2203k, YANG-2204, YANG-2205, YANG-2206, YANG-2207, and YANG-2208 as exemplified herein have been found to specifically bind to the S2 subunit of the SARS-CoV-2 spike protein.

In a first aspect, the present invention provides an antibody that specifically binds to the S2 subunit of the SARS-CoV-2 spike protein.

In one embodiment, the antibody specifically binds the S2 subunit of the SARS-CoV-2 spike protein with a KD of 0.1 nM or lower (e.g. as measured by surface plasmon resonance (SPR)).

In one embodiment, the antibody comprises a variable heavy (VH) domain sequence comprising complementarity determining regions (CDRs) HCDR1, HCDR2 and HCDR3, and a variable light (VL) domain sequence comprising complementarity determining regions LCDR1, LCDR2 and LCDR3, wherein the HCDR3 is the HCDR3 of antibody YANG-1201, YANG-1202, YANG-1203, YANG-1204, YANG-1205, YANG-1206, YANG-1207, YANG-2201, YANG-2202, YANG-2203, YANG-2203a, YANG-2203b, YANG-2203c, YANG-2203d, YANG-2203e, YANG-2203f, YANG-2203g, YANG-2203h, YANG-2203i, YANG-2203j, YANG-2203k, YANG-2204, YANG-2205, YANG-2206, YANG-2207, or YANG-2208.

In one embodiment, the antibody comprises a variable heavy (VH) domain sequence comprising complementarity determining regions (CDRs) HCDR1, HCDR2 and HCDR3, and a variable light (VL) domain sequence comprising complementarity determining regions LCDR1, LCDR2 and LCDR3, wherein HCDR3 is the HCDR3 of antibody YANG-2203, YANG-2204, YANG-2205, YANG-2206, YANG-2207, or YANG-2208.

In one embodiment, HCDR3 is the HCDR3 of antibody YANG-1201.

In one embodiment, HCDR3 is the HCDR3 of antibody YANG-1202.

In one embodiment, HCDR3 is the HCDR3 of antibody YANG-1203.

In one embodiment, HCDR3 is the HCDR3 of antibody YANG-1204.

In one embodiment, HCDR3 is the HCDR3 of antibody YANG-1205.

In one embodiment, HCDR3 is the HCDR3 of antibody YANG-1206.

In one embodiment, HCDR3 is the HCDR3 of antibody YANG-1207.

In one embodiment, HCDR3 is the HCDR3 of antibody YANG-2201.

In one embodiment, HCDR3 is the HCDR3 of antibody YANG-2202.

In one embodiment, HCDR3 is the HCDR3 of antibody YANG-2203.

In one embodiment, HCDR3 is the HCDR3 of antibody YANG-2204.

In one embodiment, HCDR3 is the HCDR3 of antibody YANG-2205.

In one embodiment, HCDR3 is the HCDR3 of antibody YANG-2206.

In one embodiment, HCDR3 is the HCDR3 of antibody YANG-2207.

In one embodiment, HCDR3 is the HCDR3 of antibody YANG-2208.

In one embodiment, the antibody specifically binds to the S2 subunit of the SARS-CoV-2 spike protein with a KD of 0.1 nM or lower (e.g. as measured by surface plasmon resonance (SPR)) and HCDR3 is the HCDR3 of antibody YANG-1201, YANG-1202, YANG-1203, YANG-1204, YANG-1205, YANG-1206, YANG-1207, YANG-2201, YANG-2202, YANG-2203, YANG-2203a, YANG-2203b, YANG-2203c, YANG-2203d, YANG-2203e, YANG-2203f, YANG-2203g, YANG-2203h, YANG-2203i, YANG-2203j, YANG-2203k, YANG-2204, YANG-2205, YANG-2206, YANG-2207, or YANG-2208.

In one embodiment, the antibody specifically binds to the S2 subunit of the SARS-CoV-2 spike protein with a KD of 0.1 nM or lower (e.g. as measured by surface plasmon resonance (SPR)) and HCDR3 is the HCDR3 of antibody YANG-2203, YANG-2204, YANG-2205, YANG-2206, YANG-2207, YANG-2208.

In one embodiment, the antibody specifically binds to the S2 subunit of the SARS-CoV-2 spike protein and neutralises SARS-CoV-2 with at least 35% neutralisation (e.g. as measured in a pseudovirus neutralisation assay), and HCDR3 is the HCDR3 of antibody YANG-2203, YANG-2204, YANG-2205, YANG-2206, YANG-2207, YANG-2208.

In one embodiment, the antibody specifically binds to the S2 subunit of the SARS-CoV-2 spike protein with a KD of 0.1 nM or lower (e.g. as measured by surface plasmon resonance (SPR)) and neutralises SARS-CoV-2 with at least 35% neutralisation (e.g. as measured in a pseudovirus neutralisation assay), and HCDR3 is the HCDR3 of antibody YANG-2203, YANG-2204, YANG-2205, YANG-2206, YANG-2207, YANG-2208.

In one embodiment, the present invention provides an anti-SARS-CoV-2, wherein the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3, wherein the CDRs are those of antibody YANG-1201, YANG-1202, YANG-1203, YANG-1204, YANG-1205, YANG-1206, YANG-1207, YANG-2201, YANG-2202, YANG-2203, YANG-2203a, YANG-2203b, YANG-2203c, YANG-2203d, YANG-2203e, YANG-2203f, YANG-2203g, YANG-2203h, YANG-2203i, YANG-2203j, YANG-2203k, YANG-2204, YANG-2205, YANG-2206, YANG-2207, or YANG-2208.

In one embodiment, the present invention provides an anti-SARS-CoV-2 antibody, wherein the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3, wherein the CDRs are those of antibody YANG-2203, YANG-2204, YANG-2205, YANG-2206, YANG-2207, or YANG-2208.

In one embodiment, the antibody has the CDRs of antibody YANG-1201.

In one embodiment, the antibody has the CDRs of antibody YANG-1202.

In one embodiment, the antibody has the CDRs of antibody YANG-1203.

In one embodiment, the antibody has the CDRs of antibody YANG-1204.

In one embodiment, the antibody has the CDRs of antibody YANG-1205.

In one embodiment, the antibody has the CDRs of antibody YANG-1206.

In one embodiment, the antibody has the CDRs of antibody YANG-1207.

In one embodiment, the antibody has the CDRs of antibody YANG-2201.

In one embodiment, the antibody has the CDRs of antibody YANG-2202.

In one embodiment, the antibody has the CDRs of antibody YANG-2203.

In one embodiment, the antibody has the CDRs of antibody YANG-2204.

In one embodiment, the antibody has the CDRs of antibody YANG-2205.

In one embodiment, the antibody has the CDRs of antibody YANG-2206.

In one embodiment, the antibody has the CDRs of antibody YANG-2207.

In one embodiment, the antibody has the CDRs of antibody YANG-2208.

In one embodiment, the present invention provides an anti-SARS-CoV-2, wherein the antibody specifically binds to the S2 subunit of the SARS-CoV-2 spike protein with a KD of 0.1 nM or lower (e.g. as measured by surface plasmon resonance (SPR)), and wherein the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3, wherein the CDRs are those of antibody YANG-1201, YANG-1202, YANG-1203, YANG-1204, YANG-1205, YANG-1206, YANG-1207, YANG-2201, YANG-2202, YANG-2203, YANG-2203a, YANG-2203b, YANG-2203c, YANG-2203d, YANG-2203e, YANG-2203f, YANG-2203g, YANG-2203h, YANG-2203i, YANG-2203j, YANG-2203k, YANG-2204, YANG-2205, YANG-2206, YANG-2207, or YANG-2208.

In one embodiment, the present invention provides an anti-SARS-CoV-2 antibody, wherein the antibody specifically binds to the S2 subunit of the SARS-CoV-2 spike protein with a KD of 0.1 nM or lower (e.g. as measured by surface plasmon resonance (SPR)), and wherein the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3, wherein the CDRs are those of antibody YANG-2203, YANG-2204, YANG-2205, YANG-2206, YANG-2207, or YANG-2208.

In one embodiment, the antibody specifically binds to the S2 subunit of the SARS-CoV-2 spike protein and neutralises SARS-CoV-2 with at least 35% neutralisation (e.g. as measured in a pseudovirus neutralisation assay), and wherein the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3, wherein the CDRs are those of antibody YANG-2203, YANG-2204, YANG-2205, YANG-2206, YANG-2207, or YANG-2208.

In one embodiment, the antibody specifically binds to the S2 subunit of the SARS-CoV-2 spike protein with a KD of 0.1 nM or lower (e.g. as measured by surface plasmon resonance (SPR)) and neutralises SARS-CoV-2 with at least 35% neutralisation (e.g. as measured in a pseudovirus neutralisation assay), and

wherein the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3, wherein the CDRs are those of antibody YANG-2203, YANG-2204, YANG-2205, YANG-2206, YANG-2207, or YANG-2208.

In one embodiment, the antibody comprises a variable heavy (VH) domain sequence and a variable light (VL) domain sequence and wherein the variable heavy (VH) domain and variable light (VL) domain sequences respectively comprise the variable heavy (VH) domain and variable light (VL) domain sequences of antibody YANG-1201, YANG-1202, YANG-1203, YANG-1204, YANG-1205, YANG-1206, YANG-1207, YANG-2201, YANG-2202, YANG-2203, YANG-2203a, YANG-2203b, YANG-2203c, YANG-2203d, YANG-2203e, YANG-2203f, YANG-2203g, YANG-2203h, YANG-2203i, YANG-2203j, YANG-2203k, YANG-2204, YANG-2205, YANG-2206, YANG-2207, or YANG-2208, YANG-2209, YANG-2210, YANG-2211, YANG-2212, YANG-2213, YANG-2214, YANG-2215, YANG-2216, YANG-2217, YANG-2218, YANG-2219, YANG-2220, YANG-2221, YANG-2222, YANG-2223, YANG-2224, YANG-2225, YANG-2226, YANG-2227, YANG-2228, YANG-2229, YANG-2230, YANG-2231, YANG-2232, YANG-2233, YANG-2234, YANG-2235, YANG-2236, YANG-2237, YANG-2238, YANG-2239, YANG-2240, YANG-2241, YANG-2242, YANG-2243, YANG-2244, YANG-2245, YANG-2246, YANG-2247, YANG-2248, YANG-2249, YANG-2250, YANG-2251, YANG-2252, YANG-2253, YANG-2254, YANG-2255, YANG-2256, YANG-2257, YANG-2258, YANG-2259, YANG-2260, YANG-2261, YANG-2262, YANG-2263, YANG-2264, YANG-2265, YANG-2266, YANG-2267, YANG-2268, YANG-2269, YANG-2270, YANG-2271, YANG-2272, YANG-2273, YANG-2274, YANG-2275, YANG-2276, YANG-2277, YANG-2278, YANG-2279, YANG-2280, YANG-2281, YANG-2282, YANG-2283, YANG-2284, YANG-2285, YANG-2286, YANG-2287, YANG-2288, YANG-2289, YANG-2290, YANG-2291, YANG-2292, YANG-2293, YANG-2294, YANG-2295, YANG-2296, YANG-2297, YANG-2298, YANG-2299, YANG-2299a, YANG-2299b, YANG-2299c, YANG-2299d, YANG-2299e, YANG-2299f, YANG-2299g, YANG-2299h, YANG-2299i, YANG-2299j, YANG-2299k, YANG-22991, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

In one embodiment, the antibody comprises a variable heavy (VH) domain sequence and a variable light (VL) domain sequence and wherein the variable heavy (VH) domain and variable light (VL) domain sequences respectively comprise the variable heavy (VH) domain and variable light (VL) domain sequences of antibody YANG-2203, YANG-2204, YANG-2205, YANG-2206, YANG-2207, or YANG-2208, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

In one embodiment, the antibody comprises a variable heavy (VH) domain sequence of antibody YANG-1201, optionally with 1, 2, 3, 4 or 5 amino acid alterations outside the complementarity determining regions (CDRs), and a variable light (VL) domain sequence of antibody YANG-1201, optionally with 1, 2, 3, 4 or 5 amino acid alterations outside the complementarity determining regions (CDRs).

In one embodiment, the antibody comprises a variable heavy (VH) domain sequence of antibody YANG-1202, optionally with 1, 2, 3, 4 or 5 amino acid alterations outside the complementarity determining regions (CDRs), and a variable light (VL) domain sequence of antibody YANG-1202, optionally with 1, 2, 3, 4 or 5 amino acid alterations outside the complementarity determining regions (CDRs).

In one embodiment, the antibody comprises a variable heavy (VH) domain sequence of antibody YANG-1203, optionally with 1, 2, 3, 4 or 5 amino acid alterations outside the complementarity determining regions (CDRs), and a variable light (VL) domain sequence of antibody YANG-1203, optionally with 1, 2, 3, 4 or 5 amino acid alterations outside the complementarity determining regions (CDRs).

In one embodiment, the antibody comprises a variable heavy (VH) domain sequence of antibody YANG-1204, optionally with 1, 2, 3, 4 or 5 amino acid alterations outside the complementarity determining regions (CDRs), and a variable light (VL) domain sequence of antibody YANG-1204, optionally with 1, 2, 3, 4 or 5 amino acid alterations outside the complementarity determining regions (CDRs).

In one embodiment, the antibody comprises a variable heavy (VH) domain sequence of antibody YANG-1205, optionally with 1, 2, 3, 4 or 5 amino acid alterations outside the complementarity determining regions (CDRs), and a variable light (VL) domain sequence of antibody YANG-1205, optionally with 1, 2, 3, 4 or 5 amino acid alterations outside the complementarity determining regions (CDRs).

In one embodiment, the antibody comprises a variable heavy (VH) domain sequence of antibody YANG-1206, optionally with 1, 2, 3, 4 or 5 amino acid alterations outside the complementarity determining regions (CDRs), and a variable light (VL) domain sequence of antibody YANG-1206, optionally with 1, 2, 3, 4 or 5 amino acid alterations outside the complementarity determining regions (CDRs).

In one embodiment, the antibody comprises a variable heavy (VH) domain sequence of antibody YANG-1207, optionally with 1, 2, 3, 4 or 5 amino acid alterations outside the complementarity determining regions (CDRs), and a variable light (VL) domain sequence of antibody YANG-1207, optionally with 1, 2, 3, 4 or 5 amino acid alterations outside the complementarity determining regions (CDRs).

In one embodiment, the antibody comprises a variable heavy (VH) domain sequence of antibody YANG-2201, optionally with 1, 2, 3, 4 or 5 amino acid alterations outside the complementarity determining regions (CDRs), and a variable light (VL) domain sequence of antibody YANG-2201, optionally with 1, 2, 3, 4 or 5 amino acid alterations outside the complementarity determining regions (CDRs).

In one embodiment, the antibody comprises a variable heavy (VH) domain sequence of antibody YANG-2202, optionally with 1, 2, 3, 4 or 5 amino acid alterations outside the complementarity determining regions (CDRs), and a variable light (VL) domain sequence of antibody YANG-2202, optionally with 1, 2, 3, 4 or 5 amino acid alterations outside the complementarity determining regions (CDRs).

In one embodiment, the antibody comprises a variable heavy (VH) domain sequence of antibody YANG-2203, optionally with 1, 2, 3, 4 or 5 amino acid alterations outside the complementarity determining regions (CDRs), and a variable light (VL) domain sequence of antibody YANG-2203, optionally with 1, 2, 3, 4 or 5 amino acid alterations outside the complementarity determining regions (CDRs).

In one embodiment, the antibody comprises a variable heavy (VH) domain sequence of antibody YANG-2204, optionally with 1, 2, 3, 4 or 5 amino acid alterations outside the complementarity determining regions (CDRs), and a variable light (VL) domain sequence of antibody YANG-2204, optionally with 1, 2, 3, 4 or 5 amino acid alterations outside the complementarity determining regions (CDRs).

In one embodiment, the antibody comprises a variable heavy (VH) domain sequence of antibody YANG-2205, optionally with 1, 2, 3, 4 or 5 amino acid alterations outside the complementarity determining regions (CDRs), and a variable light (VL) domain sequence of antibody YANG-2205, optionally with 1, 2, 3, 4 or 5 amino acid alterations outside the complementarity determining regions (CDRs).

In one embodiment, the antibody comprises a variable heavy (VH) domain sequence of antibody YANG-2206, optionally with 1, 2, 3, 4 or 5 amino acid alterations outside the complementarity determining regions (CDRs), and a variable light (VL) domain sequence of antibody YANG-2206, optionally with 1, 2, 3, 4 or 5 amino acid alterations outside the complementarity determining regions (CDRs).

In one embodiment, the antibody comprises a variable heavy (VH) domain sequence of antibody YANG-2207, optionally with 1, 2, 3, 4 or 5 amino acid alterations outside the complementarity determining regions (CDRs), and a variable light (VL) domain sequence of antibody YANG-2207, optionally with 1, 2, 3, 4 or 5 amino acid alterations outside the complementarity determining regions (CDRs).

In one embodiment, the antibody comprises a variable heavy (VH) domain sequence of antibody YANG-2208, optionally with 1, 2, 3, 4 or 5 amino acid alterations outside the complementarity determining regions (CDRs), and a variable light (VL) domain sequence of antibody YANG-2208, optionally with 1, 2, 3, 4 or 5 amino acid alterations outside the complementarity determining regions (CDRs).

In one embodiment, the antibody specifically binds the S2 subunit of the SARS-CoV-2 spike protein with a KD of 0.1 nM or lower (e.g. as measured by surface plasmon resonance (SPR)), and the variable heavy (VH) domain and variable light (VL) domain sequences respectively comprise the variable heavy (VH) domain and variable light (VL) domain sequences of antibody YANG-1201, YANG-1202, YANG-1203, YANG-1204, YANG-1205, YANG-1206, YANG-1207, YANG-2201, YANG-2202, YANG-2203, YANG-2204, YANG-2205, YANG-2206, YANG-2207, or YANG-2208, YANG-2209, YANG-2210, YANG-2211, YANG-2212, YANG-2213, YANG-2214, YANG-2215, YANG-2216, YANG-2217, YANG-2218, YANG-2219, YANG-2220, YANG-2221, YANG-2222, YANG-2223, YANG-2224, YANG-2225, YANG-2226, YANG-2227, YANG-2228, YANG-2229, YANG-2230, YANG-2231, YANG-2232, YANG-2233, YANG-2234, YANG-2235, YANG-2236, YANG-2237, YANG-2238, YANG-2239, YANG-2240, YANG-2241, YANG-2242, YANG-2243, YANG-2244, YANG-2245, YANG-2246, YANG-2247, YANG-2248, YANG-2249, YANG-2250, YANG-2251, YANG-2252, YANG-2253, YANG-2254, YANG-2255, YANG-2256, YANG-2257, YANG-2258, YANG-2259, YANG-2260, YANG-2261, YANG-2262, YANG-2263, YANG-2264, YANG-2265, YANG-2266, YANG-2267, YANG-2268, YANG-2269, YANG-2270, YANG-2271, YANG-2272, YANG-2273, YANG-2274, YANG-2275, YANG-2276, YANG-2277, YANG-2278, YANG-2279, YANG-2280, YANG-2281, YANG-2282, YANG-2283, YANG-2284, YANG-2285, YANG-2286, YANG-2287, YANG-2288, YANG-2289, YANG-2290, YANG-2291, YANG-2292, YANG-2293, YANG-2294, YANG-2295, YANG-2296, YANG-2297, YANG-2298, YANG-2299, YANG-2299a, YANG-2299b, YANG-2299c, YANG-2299d, YANG-2299e, YANG-2299f, YANG-2299g, YANG-2299h, YANG-2299i, YANG-2299j, YANG-2299k, YANG-22991, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

In one embodiment, the antibody specifically binds the S2 subunit of the SARS-CoV-2 spike protein with a KD of 0.1 nM or lower (e.g. as measured by surface plasmon resonance (SPR)), and the variable heavy (VH) domain and variable light (VL) domain sequences respectively comprise the variable heavy (VH) domain and variable light (VL) domain sequences of antibody YANG-2203, YANG-2204, YANG-2205, YANG-2206, YANG-2207, or YANG-2208, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

In one embodiment, the antibody specifically binds to the S2 subunit of the SARS-CoV-2 spike protein and neutralises SARS-CoV-2 with at least 35% neutralisation (e.g. as measured in a pseudovirus neutralisation assay), and the variable heavy (VH) domain and variable light (VL) domain sequences respectively comprise the variable heavy (VH) domain and variable light (VL) domain sequences of antibody YANG-2203, YANG-2204, YANG-2205, YANG-2206, YANG-2207, or YANG-2208, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

In one embodiment, the antibody specifically binds to the S2 subunit of the SARS-CoV-2 spike protein with a KD of 0.1 nM or lower (e.g. as measured by surface plasmon resonance (SPR)) and neutralises SARS-CoV-2 with at least 35% neutralisation (e.g. as measured in a pseudovirus neutralisation assay), and the variable heavy (VH) domain and variable light (VL) domain sequences respectively comprise the variable heavy (VH) domain and variable light (VL) domain sequences of antibody YANG-2203, YANG-2204, YANG-2205, YANG-2206, YANG-2207, or YANG-2208, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

In one embodiment, the antibody comprises a variable heavy (VH) domain sequence and a variable light (VL) domain sequence and wherein the variable heavy (VH) domain and variable light (VL) domain sequences respectively comprise a sequence having at least 90% identity to the variable heavy (VH) and variable light (VL) domain sequences of antibody YANG-1201, YANG-1202, YANG-1203, YANG-1204, YANG-1205, YANG-1206, YANG-1207, YANG-2201, YANG-2202, YANG-2203, YANG-2203a, YANG-2203b, YANG-2203c, YANG-2203d, YANG-2203e, YANG-2203f, YANG-2203g, YANG-2203h, YANG-2203i, YANG-2203j, YANG-2203k, YANG-2204, YANG-2205, YANG-2206, YANG-2207, or YANG-2208, YANG-2209, YANG-2210, YANG-2211, YANG-2212, YANG-2213, YANG-2214, YANG-2215, YANG-2216, YANG-2217, YANG-2218, YANG-2219, YANG-2220, YANG-2221, YANG-2222, YANG-2223, YANG-2224, YANG-2225, YANG-2226, YANG-2227, YANG-2228, YANG-2229, YANG-2230, YANG-2231, YANG-2232, YANG-2233, YANG-2234, YANG-2235, YANG-2236, YANG-2237, YANG-2238, YANG-2239, YANG-2240, YANG-2241, YANG-2242, YANG-2243, YANG-2244, YANG-2245, YANG-2246, YANG-2247, YANG-2248, YANG-2249, YANG-2250, YANG-2251, YANG-2252, YANG-2253, YANG-2254, YANG-2255, YANG-2256, YANG-2257, YANG-2258, YANG-2259, YANG-2260, YANG-2261, YANG-2262, YANG-2263, YANG-2264, YANG-2265, YANG-2266, YANG-2267, YANG-2268, YANG-2269, YANG-2270, YANG-2271, YANG-2272, YANG-2273, YANG-2274, YANG-2275, YANG-2276, YANG-2277, YANG-2278, YANG-2279, YANG-2280, YANG-2281, YANG-2282, YANG-2283, YANG-2284, YANG-2285, YANG-2286, YANG-2287, YANG-2288, YANG-2289, YANG-2290, YANG-2291, YANG-2292, YANG-2293, YANG-2294, YANG-2295, YANG-2296, YANG-2297, YANG-2298, YANG-2299, YANG-2299a, YANG-2299b, YANG-2299c, YANG-2299d, YANG-2299e, YANG-2299f, YANG-2299g, YANG-2299h, YANG-2299i, YANG-2299j, YANG-2299k, YANG-22991, provided that the antibody has the CDRs of antibody YANG-1201, YANG-1202, YANG-1203, YANG-1204, YANG-1205, YANG-1206, YANG-1207, YANG-2201, YANG-2202, YANG-2203, YANG-2203a, YANG-2203b, YANG-2203c, YANG-2203d, YANG-2203e, YANG-2203f, YANG-2203g, YANG-2203h, YANG-2203i, YANG-2203j, YANG-2203k, YANG-2204, YANG-2205, YANG-2206, YANG-2207, or YANG-2208, YANG-2209, YANG-2210, YANG-2211, YANG-2212, YANG-2213, YANG-2214, YANG-2215, YANG-2216, YANG-2217, YANG-2218, YANG-2219, YANG-2220, YANG-2221, YANG-2222, YANG-2223, YANG-2224, YANG-2225, YANG-2226, YANG-2227, YANG-2228, YANG-2229, YANG-2230, YANG-2231, YANG-2232, YANG-2233, YANG-2234, YANG-2235, YANG-2236, YANG-2237, YANG-2238, YANG-2239, YANG-2240, YANG-2241, YANG-2242, YANG-2243, YANG-2244, YANG-2245, YANG-2246, YANG-2247, YANG-2248, YANG-2249, YANG-2250, YANG-2251, YANG-2252, YANG-2253, YANG-2254, YANG-2255, YANG-2256, YANG-2257, YANG-2258, YANG-2259, YANG-2260, YANG-2261, YANG-2262, YANG-2263, YANG-2264, YANG-2265, YANG-2266, YANG-2267, YANG-2268, YANG-2269, YANG-2270, YANG-2271, YANG-2272, YANG-2273, YANG-2274, YANG-2275, YANG-2276, YANG-2277, YANG-2278, YANG-2279, YANG-2280, YANG-2281, YANG-2282, YANG-2283, YANG-2284, YANG-2285, YANG-2286, YANG-2287, YANG-2288, YANG-2289, YANG-2290, YANG-2291, YANG-2292, YANG-2293, YANG-2294, YANG-2295, YANG-2296, YANG-2297, YANG-2298, YANG-2299, YANG-2299a, YANG-2299b, YANG-2299c, YANG-2299d, YANG-2299e, YANG-2299f, YANG-2299g, YANG-2299h, YANG-2299i, YANG-2299j, YANG-2299k, YANG-22991, respectively.

In one embodiment, the antibody comprises a variable heavy (VH) domain sequence and a variable light (VL) domain sequence and wherein the variable heavy (VH) domain and variable light (VL) domain sequences respectively comprise a sequence having at least 90% identity to the variable heavy (VH) and variable light (VL) domain sequences of antibody YANG-2203, YANG-2204, YANG-2205, YANG-2206, YANG-2207, or YANG-2208, provided that the antibody has the CDRs of antibody YANG-2203, YANG-2204, YANG-2205, YANG-2206, YANG-2207, or YANG-2208, respectively.

In one embodiment, the variable heavy (VH) domain sequence comprises a sequence having at least 90% identity to the VH domain sequence of antibody YANG-1201 and the variable light (VL) domain sequence comprises a sequence having at least 90% identity to the VL domain sequence of antibody YANG-1201, provided that the antibody has the CDRs of antibody YANG-1201.

In one embodiment, the variable heavy (VH) domain sequence comprises a sequence having at least 90% identity to the VH domain sequence of antibody YANG-1202 and the variable light (VL) domain sequence comprises a sequence having at least 90% identity to the VL domain sequence of antibody YANG-1202, provided that the antibody has the CDRs of antibody YANG-1202.

In one embodiment, the variable heavy (VH) domain sequence comprises a sequence having at least 90% identity to the VH domain sequence of antibody YANG-1203 and the variable light (VL) domain sequence comprises a sequence having at least 90% identity to the VL domain sequence of antibody YANG-1203, provided that the antibody has the CDRs of antibody YANG-1203.

In one embodiment, the variable heavy (VH) domain sequence comprises a sequence having at least 90% identity to the VH domain sequence of antibody YANG-1204 and the variable light (VL) domain sequence comprises a sequence having at least 90% identity to the VL domain sequence of antibody YANG-1204, provided that the antibody has the CDRs of antibody YANG-1204.

In one embodiment, the variable heavy (VH) domain sequence comprises a sequence having at least 90% identity to the VH domain sequence of antibody YANG-1205 and the variable light (VL) domain sequence comprises a sequence having at least 90% identity to the VL domain sequence of antibody YANG-1205, provided that the antibody has the CDRs of antibody YANG-1205.

In one embodiment, the variable heavy (VH) domain sequence comprises a sequence having at least 90% identity to the VH domain sequence of antibody YANG-1206 and the variable light (VL) domain sequence comprises a sequence having at least 90% identity to the VL domain sequence of antibody YANG-1206, provided that the antibody has the CDRs of antibody YANG-1206.

In one embodiment, the variable heavy (VH) domain sequence comprises a sequence having at least 90% identity to the VH domain sequence of antibody YANG-1207 and the variable light (VL) domain sequence comprises a sequence having at least 90% identity to the VL domain sequence of antibody YANG-1207, provided that the antibody has the CDRs of antibody YANG-1207.

In one embodiment, the variable heavy (VH) domain sequence comprises a sequence having at least 90% identity to the VH domain sequence of antibody YANG-2201 and the variable light (VL) domain sequence comprises a sequence having at least 90% identity to the VL domain sequence of antibody YANG-2201, provided that the antibody has the CDRs of antibody YANG-2201.

In one embodiment, the variable heavy (VH) domain sequence comprises a sequence having at least 90% identity to the VH domain sequence of antibody YANG-2202 and the variable light (VL) domain sequence comprises a sequence having at least 90% identity to the VL domain sequence of antibody YANG-2202, provided that the antibody has the CDRs of antibody YANG-2202.

In one embodiment, the variable heavy (VH) domain sequence comprises a sequence having at least 90% identity to the VH domain sequence of antibody YANG-2203 and the variable light (VL) domain sequence comprises a sequence having at least 90% identity to the VL domain sequence of antibody YANG-2203, provided that the antibody has the CDRs of antibody YANG-2203.

In one embodiment, the variable heavy (VH) domain sequence comprises a sequence having at least 90% identity to the VH domain sequence of antibody YANG-2204 and the variable light (VL) domain sequence comprises a sequence having at least 90% identity to the VL domain sequence of antibody YANG-2204, provided that the antibody has the CDRs of antibody YANG-2204.

In one embodiment, the variable heavy (VH) domain sequence comprises a sequence having at least 90% identity to the VH domain sequence of antibody YANG-2205 and the variable light (VL) domain sequence comprises a sequence having at least 90% identity to the VL domain sequence of antibody YANG-2205, provided that the antibody has the CDRs of antibody YANG-2205.

In one embodiment, the variable heavy (VH) domain sequence comprises a sequence having at least 90% identity to the VH domain sequence of antibody YANG-2206 and the variable light (VL) domain sequence comprises a sequence having at least 90% identity to the VL domain sequence of antibody YANG-2206, provided that the antibody has the CDRs of antibody YANG-2206.

In one embodiment, the variable heavy (VH) domain sequence comprises a sequence having at least 90% identity to the VH domain sequence of antibody YANG-2207 and the variable light (VL) domain sequence comprises a sequence having at least 90% identity to the VL domain sequence of antibody YANG-2207, provided that the antibody has the CDRs of antibody YANG-2207.

In one embodiment, the variable heavy (VH) domain sequence comprises a sequence having at least 90% identity to the VH domain sequence of antibody YANG-2208 and the variable light (VL) domain sequence comprises a sequence having at least 90% identity to the VL domain sequence of antibody YANG-2208, provided that the antibody has the CDRs of antibody YANG-2208.

In one embodiment, the antibody specifically binds the S2 subunit of the SARS-CoV-2 spike protein with a KD of 0.1 nM or lower (e.g. as measured by surface plasmon resonance (SPR)), and the antibody comprises a variable heavy (VH) domain sequence and a variable light (VL) domain sequence and wherein the variable heavy (VH) domain and variable light (VL) domain sequences respectively comprise a sequence having at least 90% identity to the variable heavy (VH) and variable light (VL) domain sequences of antibody YANG-1201, YANG-1202, YANG-1203, YANG-1204, YANG-1205, YANG-1206, YANG-1207, YANG-2201, YANG-2202, YANG-2203, YANG-2203a, YANG-2203b, YANG-2203c, YANG-2203d, YANG-2203e, YANG-2203f, YANG-2203g, YANG-2203h, YANG-2203i, YANG-2203j, YANG-2203k, YANG-2204, YANG-2205, YANG-2206, YANG-2207, or YANG-2208, YANG-2209, YANG-2210, YANG-2211, YANG-2212, YANG-2213, YANG-2214, YANG-2215, YANG-2216, YANG-2217, YANG-2218, YANG-2219, YANG-2220, YANG-2221, YANG-2222, YANG-2223, YANG-2224, YANG-2225, YANG-2226, YANG-2227, YANG-2228, YANG-2229, YANG-2230, YANG-2231, YANG-2232, YANG-2233, YANG-2234, YANG-2235, YANG-2236, YANG-2237, YANG-2238, YANG-2239, YANG-2240, YANG-2241, YANG-2242, YANG-2243, YANG-2244, YANG-2245, YANG-2246, YANG-2247, YANG-2248, YANG-2249, YANG-2250, YANG-2251, YANG-2252, YANG-2253, YANG-2254, YANG-2255, YANG-2256, YANG-2257, YANG-2258, YANG-2259, YANG-2260, YANG-2261, YANG-2262, YANG-2263, YANG-2264, YANG-2265, YANG-2266, YANG-2267, YANG-2268, YANG-2269, YANG-2270, YANG-2271, YANG-2272, YANG-2273, YANG-2274, YANG-2275, YANG-2276, YANG-2277, YANG-2278, YANG-2279, YANG-2280, YANG-2281, YANG-2282, YANG-2283, YANG-2284, YANG-2285, YANG-2286, YANG-2287, YANG-2288, YANG-2289, YANG-2290, YANG-2291, YANG-2292, YANG-2293, YANG-2294, YANG-2295, YANG-2296, YANG-2297, YANG-2298, YANG-2299, YANG-2299a, YANG-2299b, YANG-2299c, YANG-2299d, YANG-2299e, YANG-2299f, YANG-2299g, YANG-2299h, YANG-2299i, YANG-2299j, YANG-2299k, YANG-22991, provided that the antibody has the CDRs of antibody YANG-1201, YANG-1202, YANG-1203, YANG-1204, YANG-1205, YANG-1206, YANG-1207, YANG-2201, YANG-2202, YANG-2203, YANG-2203a, YANG-2203b, YANG-2203c, YANG-2203d, YANG-2203e, YANG-2203f, YANG-2203g, YANG-2203h, YANG-2203i, YANG-2203j, YANG-2203k, YANG-2204, YANG-2205, YANG-2206, YANG-2207, or YANG-2208, YANG-2209, YANG-2210, YANG-2211, YANG-2212, YANG-2213, YANG-2214, YANG-2215, YANG-2216, YANG-2217, YANG-2218, YANG-2219, YANG-2220, YANG-2221, YANG-2222, YANG-2223, YANG-2224, YANG-2225, YANG-2226, YANG-2227, YANG-2228, YANG-2229, YANG-2230, YANG-2231, YANG-2232, YANG-2233, YANG-2234, YANG-2235, YANG-2236, YANG-2237, YANG-2238, YANG-2239, YANG-2240, YANG-2241, YANG-2242, YANG-2243, YANG-2244, YANG-2245, YANG-2246, YANG-2247, YANG-2248, YANG-2249, YANG-2250, YANG-2251, YANG-2252, YANG-2253, YANG-2254, YANG-2255, YANG-2256, YANG-2257, YANG-2258, YANG-2259, YANG-2260, YANG-2261, YANG-2262, YANG-2263, YANG-2264, YANG-2265, YANG-2266, YANG-2267, YANG-2268, YANG-2269, YANG-2270, YANG-2271, YANG-2272, YANG-2273, YANG-2274, YANG-2275, YANG-2276, YANG-2277, YANG-2278, YANG-2279, YANG-2280, YANG-2281, YANG-2282, YANG-2283, YANG-2284, YANG-2285, YANG-2286, YANG-2287, YANG-2288, YANG-2289, YANG-2290, YANG-2291, YANG-2292, YANG-2293, YANG-2294, YANG-2295, YANG-2296, YANG-2297, YANG-2298, YANG-2299, YANG-2299a, YANG-2299b, YANG-2299c, YANG-2299d, YANG-2299e, YANG-2299f, YANG-2299g, YANG-2299h, YANG-2299i, YANG-2299j, YANG-2299k, YANG-22991, respectively.

In one embodiment, the antibody specifically binds the S2 subunit of the SARS-CoV-2 spike protein with a KD of 0.1 nM or lower (e.g. as measured by surface plasmon resonance (SPR)), and the antibody comprises a variable heavy (VH) domain sequence and a variable light (VL) domain sequence and wherein the variable heavy (VH) domain and variable light (VL) domain sequences respectively comprise a sequence having at least 90% identity to the variable heavy (VH) and variable light (VL) domain sequences of antibody YANG-2203, YANG-2204, YANG-2205, YANG-2206, YANG-2207, or YANG-2208, provided that the antibody has the CDRs of antibody YANG-2203, YANG-2204, YANG-2205, YANG-2206, YANG-2207, or YANG-2208, respectively.

In one embodiment, the antibody specifically binds to the S2 subunit of the SARS-CoV-2 spike protein and neutralises SARS-CoV-2 with at least 35% neutralisation (e.g. as measured in a pseudovirus neutralisation assay), and the antibody comprises a variable heavy (VH) domain sequence and a variable light (VL) domain sequence and wherein the variable heavy (VH) domain and variable light (VL) domain sequences respectively comprise a sequence having at least 90% identity to the variable heavy (VH) and variable light (VL) domain sequences of antibody YANG-2203, YANG-2204, YANG-2205, YANG-2206, YANG-2207, or YANG-2208, provided that the antibody has the CDRs of antibody YANG-2203, YANG-2204, YANG-2205, YANG-2206, YANG-2207, or YANG-2208, respectively.

In one embodiment, the antibody specifically binds to the S2 subunit of the SARS-CoV-2 spike protein with a KD of 0.1 nM or lower (e.g. as measured by surface plasmon resonance (SPR)) and neutralises SARS-CoV-2 with at least 35% neutralisation (e.g. as measured in a pseudovirus neutralisation assay), and the antibody comprises a variable heavy (VH) domain sequence and a variable light (VL) domain sequence and wherein the variable heavy (VH) domain and variable light (VL) domain sequences respectively comprise a sequence having at least 90% identity to the variable heavy (VH) and variable light (VL) domain sequences of antibody YANG-2203, YANG-2204, YANG-2205, YANG-2206, YANG-2207, or YANG-2208, provided that the antibody has the CDRs of antibody YANG-2203, YANG-2204, YANG-2205, YANG-2206, YANG-2207, or YANG-2208, respectively.

In one embodiment, the present invention provides an antibody that specifically binds to the S2 subunit of the SARS-CoV-2 spike protein, wherein the antibody comprises a variable heavy (VH) domain sequence and a variable light (VL) domain sequence and wherein the variable heavy (VH) domain and variable light (VL) domain sequences respectively comprise the variable heavy (VH) and variable light (VL) domain sequences of antibody YANG-1201, YANG-1202, YANG-1203, YANG-1204, YANG-1205, YANG-1206, YANG-1207, YANG-2201, YANG-2202, YANG-2203, YANG-2203a, YANG-2203b, YANG-2203c, YANG-2203d, YANG-2203e, YANG-2203f, YANG-2203g, YANG-2203h, YANG-2203i, YANG-2203j, YANG-2203k, YANG-2204, YANG-2205, YANG-2206, YANG-2207, or YANG-2208.

In one embodiment, the present invention provides an antibody that specifically binds to the S2 subunit of the SARS-CoV-2 spike protein, wherein the antibody comprises a variable heavy (VH) domain sequence and a variable light (VL) domain sequence and wherein the variable heavy (VH) domain and variable light (VL) domain sequences respectively comprise the variable heavy (VH) and variable light (VL) domain sequences of antibody YANG-2203, YANG-2204, YANG-2205, YANG-2206, YANG-2207, or YANG-2208.

An antibody that specifically binds to the S2 subunit of the SARS-CoV-2 spike protein, wherein the antibody comprises the VH and VL domain sequences of antibody YANG-1201.

An antibody that specifically binds to the S2 subunit of the SARS-CoV-2 spike protein, wherein the antibody comprises the VH and VL domain sequences of antibody YANG-1202.

An antibody that specifically binds to the S2 subunit of the SARS-CoV-2 spike protein, wherein the antibody comprises the VH and VL domain sequences of antibody YANG-1203.

An antibody that specifically binds to the S2 subunit of the SARS-CoV-2 spike protein, wherein the antibody comprises the VH and VL domain sequences of antibody YANG-1204.

An antibody that specifically binds to the S2 subunit of the SARS-CoV-2 spike protein, wherein the antibody comprises the VH and VL domain sequences of antibody YANG-1205.

An antibody that specifically binds to the S2 subunit of the SARS-CoV-2 spike protein, wherein the antibody comprises the VH and VL domain sequences of antibody YANG-1206.

An antibody that specifically binds to the S2 subunit of the SARS-CoV-2 spike protein, wherein the antibody comprises the VH and VL domain sequences of antibody YANG-1207.

An antibody that specifically binds to the S2 subunit of the SARS-CoV-2 spike protein, wherein the antibody comprises the VH and VL domain sequences of antibody YANG-2201.

An antibody that specifically binds to the S2 subunit of the SARS-CoV-2 spike protein, wherein the antibody comprises the VH and VL domain sequences of antibody YANG-2202.

An antibody that specifically binds to the S2 subunit of the SARS-CoV-2 spike protein, wherein the antibody comprises the VH and VL domain sequences of antibody YANG-2203.

An antibody that specifically binds to the S2 subunit of the SARS-CoV-2 spike protein, wherein the antibody comprises the VH and VL domain sequences of antibody YANG-2204.

An antibody that specifically binds to the S2 subunit of the SARS-CoV-2 spike protein, wherein the antibody comprises the VH and VL domain sequences of antibody YANG-2205.

An antibody that specifically binds to the S2 subunit of the SARS-CoV-2 spike protein, wherein the antibody comprises the VH and VL domain sequences of antibody YANG-2206.

An antibody that specifically binds to the S2 subunit of the SARS-CoV-2 spike protein, wherein the antibody comprises the VH and VL domain sequences of antibody YANG-2207.

An antibody that specifically binds to the S2 subunit of the SARS-CoV-2 spike protein, wherein the antibody comprises the VH and VL domain sequences of antibody YANG-2208.

In one embodiment, the antibody comprises a variable heavy (VH) domain sequence and a variable light (VL) domain sequence and wherein the variable heavy (VH) domain and variable light (VL) domain sequences respectively comprise the variable heavy (VH) and variable light (VL) domain sequences of antibody YANG-1201, YANG-1202, YANG-1203, YANG-1204, YANG-1205, YANG-1206, YANG-1207, YANG-2201, YANG-2202, YANG-2203, YANG-2203a, YANG-2203b, YANG-2203c, YANG-2203d, YANG-2203e, YANG-2203f, YANG-2203g, YANG-2203h, YANG-2203i, YANG-2203j, YANG-2203k, YANG-2204, YANG-2205, YANG-2206, YANG-2207, or YANG-2208.

In one embodiment, the antibody comprises a variable heavy (VH) domain sequence and a variable light (VL) domain sequence and wherein the variable heavy (VH) domain and variable light (VL) domain sequences respectively comprise the variable heavy (VH) and variable light (VL) domain sequences of antibody YANG-2203, YANG-2204, YANG-2205, YANG-2206, YANG-2207, or YANG-2208.

In one embodiment, the antibody comprises the VH and VL domain sequences of antibody YANG-1201.

In one embodiment, the antibody comprises the VH and VL domain sequences of antibody YANG-1202.

In one embodiment, the antibody comprises the VH and VL domain sequences of antibody YANG-1203.

In one embodiment, the antibody comprises the VH and VL domain sequences of antibody YANG-1204.

In one embodiment, the antibody comprises the VH and VL domain sequences of antibody YANG-1205.

In one embodiment, the antibody comprises the VH and VL domain sequences of antibody YANG-1206.

In one embodiment, the antibody comprises the VH and VL domain sequences of antibody YANG-1207.

In one embodiment, the antibody comprises the VH and VL domain sequences of antibody YANG-2201.

In one embodiment, the antibody comprises the VH and VL domain sequences of antibody YANG-2202.

In one embodiment, the antibody comprises the VH and VL domain sequences of antibody YANG-2203.

In one embodiment, the antibody comprises the VH and VL domain sequences of antibody YANG-2204.

In one embodiment, the antibody comprises the VH and VL domain sequences of antibody YANG-2205.

In one embodiment, the antibody comprises the VH and VL domain sequences of antibody YANG-2206.

In one embodiment, the antibody comprises the VH and VL domain sequences of antibody YANG-2207.

In one embodiment, the antibody comprises the VH and VL domain sequences of antibody YANG-2208.

Antibodies are provided which bind to the same epitope on the S2 subunit of the SARS-CoV-2 spike protein as an antibody described anywhere herein.

An antibody is provided which bind to the same epitope as antibody YANG-1201, YANG-1202, YANG-1203, YANG-1204, YANG-1205, YANG-1206, YANG-1207, YANG-2201, YANG-2202, YANG-2203, YANG-2203a, YANG-2203b, YANG-2203c, YANG-2203d, YANG-2203e, YANG-2203f, YANG-2203g, YANG-2203h, YANG-2203i, YANG-2203j, YANG-2203k, YANG-2204, YANG-2205, YANG-2206, YANG-2207, or YANG-2208, YANG-2209, YANG-2210, YANG-2211, YANG-2212, YANG-2213, YANG-2214, YANG-2215, YANG-2216, YANG-2217, YANG-2218, YANG-2219, YANG-2220, YANG-2221, YANG-2222, YANG-2223, YANG-2224, YANG-2225, YANG-2226, YANG-2227, YANG-2228, YANG-2229, YANG-2230, YANG-2231, YANG-2232, YANG-2233, YANG-2234, YANG-2235, YANG-2236, YANG-2237, YANG-2238, YANG-2239, YANG-2240, YANG-2241, YANG-2242, YANG-2243, YANG-2244, YANG-2245, YANG-2246, YANG-2247, YANG-2248, YANG-2249, YANG-2250, YANG-2251, YANG-2252, YANG-2253, YANG-2254, YANG-2255, YANG-2256, YANG-2257, YANG-2258, YANG-2259, YANG-2260, YANG-2261, YANG-2262, YANG-2263, YANG-2264, YANG-2265, YANG-2266, YANG-2267, YANG-2268, YANG-2269, YANG-2270, YANG-2271, YANG-2272, YANG-2273, YANG-2274, YANG-2275, YANG-2276, YANG-2277, YANG-2278, YANG-2279, YANG-2280, YANG-2281, YANG-2282, YANG-2283, YANG-2284, YANG-2285, YANG-2286, YANG-2287, YANG-2288, YANG-2289, YANG-2290, YANG-2291, YANG-2292, YANG-2293, YANG-2294, YANG-2295, YANG-2296, YANG-2297, YANG-2298, YANG-2299, YANG-2299a, YANG-2299b, YANG-2299c, YANG-2299d, YANG-2299e, YANG-2299f, YANG-2299g, YANG-2299h, YANG-2299i, YANG-2299j, YANG-2299k, YANG-22991, e.g. as defined by its VH and VL sequences.

An antibody is provided which bind to the same epitope as antibody YANG-2203, YANG-2204, YANG-2205, YANG-2206, YANG-2207, or YANG-2208, e.g. as defined by its VH and VL sequences.

In one embodiment, an antibody is provided which binds to the same epitope as antibody YANG-1201.

In one embodiment, an antibody is provided which binds to the same epitope as antibody YANG-1202.

In one embodiment, an antibody is provided which binds to the same epitope as antibody YANG-1203.

In one embodiment, an antibody is provided which binds to the same epitope as antibody YANG-1204.

In one embodiment, an antibody is provided which binds to the same epitope as antibody YANG-1205.

In one embodiment, an antibody is provided which binds to the same epitope as antibody YANG-1206.

In one embodiment, an antibody is provided which binds to the same epitope as antibody YANG-1207.

In one embodiment, an antibody is provided which binds to the same epitope as antibody YANG-2201.

In one embodiment, an antibody is provided which binds to the same epitope as antibody YANG-2202.

In one embodiment, an antibody is provided which binds to the same epitope as antibody YANG-2203.

In one embodiment, an antibody is provided which binds to the same epitope as antibody YANG-2204.

In one embodiment, an antibody is provided which binds to the same epitope as antibody YANG-2205.

In one embodiment, an antibody is provided which binds to the same epitope as antibody YANG-2206.

In one embodiment, an antibody is provided which binds to the same epitope as antibody YANG-2207.

In one embodiment, an antibody is provided which binds to the same epitope as antibody YANG-2208.

An antibody may contact the SARS-CoV-2 spike protein with a footprint that fully or partly overlaps with that of an antibody disclosed anywhere herein. As described elsewhere herein, competition between antibodies may be determined, for example using SPR, and antibodies are provided which compete for binding to the spike protein (compete for binding to their epitope) with an IgG antibody as described anywhere herein.

An antibody of the present invention may be one which competes for binding to SARS-CoV-2 spike protein with any anti-S2 antibody described herein, such as YANG-1201, YANG-1202, YANG-1203, YANG-1204, YANG-1205, YANG-1206, YANG-1207, YANG-2201, YANG-2202, YANG-2203, YANG-2203a, YANG-2203b, YANG-2203c, YANG-2203d, YANG-2203e, YANG-2203f, YANG-2203g, YANG-2203h, YANG-2203i, YANG-2203j, YANG-2203k, YANG-2204, YANG-2205, YANG-2206, YANG-2207, or YANG-2208, YANG-2209, YANG-2210, YANG-2211, YANG-2212, YANG-2213, YANG-2214, YANG-2215, YANG-2216, YANG-2217, YANG-2218, YANG-2219, YANG-2220, YANG-2221, YANG-2222, YANG-2223, YANG-2224, YANG-2225, YANG-2226, YANG-2227, YANG-2228, YANG-2229, YANG-2230, YANG-2231, YANG-2232, YANG-2233, YANG-2234, YANG-2235, YANG-2236, YANG-2237, YANG-2238, YANG-2239, YANG-2240, YANG-2241, YANG-2242, YANG-2243, YANG-2244, YANG-2245, YANG-2246, YANG-2247, YANG-2248, YANG-2249, YANG-2250, YANG-2251, YANG-2252, YANG-2253, YANG-2254, YANG-2255, YANG-2256, YANG-2257, YANG-2258, YANG-2259, YANG-2260, YANG-2261, YANG-2262, YANG-2263, YANG-2264, YANG-2265, YANG-2266, YANG-2267, YANG-2268, YANG-2269, YANG-2270, YANG-2271, YANG-2272, YANG-2273, YANG-2274, YANG-2275, YANG-2276, YANG-2277, YANG-2278, YANG-2279, YANG-2280, YANG-2281, YANG-2282, YANG-2283, YANG-2284, YANG-2285, YANG-2286, YANG-2287, YANG-2288, YANG-2289, YANG-2290, YANG-2291, YANG-2292, YANG-2293, YANG-2294, YANG-2295, YANG-2296, YANG-2297, YANG-2298, YANG-2299, YANG-2299a, YANG-2299b, YANG-2299c, YANG-2299d, YANG-2299e, YANG-2299f, YANG-2299g, YANG-2299h, YANG-2299i, YANG-2299j, YANG-2299k, YANG-22991, e.g. as defined by its VH and VL sequences.

An antibody of the present invention may be one which competes for binding to the SARS-CoV-2 spike protein with antibody YANG-1201.

An antibody of the present invention may be one which competes for binding to the SARS-CoV-2 spike protein with antibody YANG-1202.

An antibody of the present invention may be one which competes for binding to the SARS-CoV-2 spike protein with antibody YANG-1203.

An antibody of the present invention may be one which competes for binding to the SARS-CoV-2 spike protein with antibody YANG-1204.

An antibody of the present invention may be one which competes for binding to the SARS-CoV-2 spike protein with antibody YANG-1205.

An antibody of the present invention may be one which competes for binding to the SARS-CoV-2 spike protein with antibody YANG-1206.

An antibody of the present invention may be one which competes for binding to the SARS-CoV-2 spike protein with antibody YANG-1207.

An antibody of the present invention may be one which competes for binding to the SARS-CoV-2 spike protein with antibody YANG-2201.

An antibody of the present invention may be one which competes for binding to the SARS-CoV-2 spike protein with antibody YANG-2202.

An antibody of the present invention may be one which competes for binding to the SARS-CoV-2 spike protein with antibody YANG-2203.

An antibody of the present invention may be one which competes for binding to the SARS-CoV-2 spike protein with antibody YANG-2204.

An antibody of the present invention may be one which competes for binding to the SARS-CoV-2 spike protein with antibody YANG-2205.

An antibody of the present invention may be one which competes for binding to the SARS-CoV-2 spike protein with antibody YANG-2206.

An antibody of the present invention may be one which competes for binding to the SARS-CoV-2 spike protein with antibody YANG-2207.

An antibody of the present invention may be one which competes for binding to the SARS-CoV-2 spike protein with antibody YANG-2208.

In one embodiment, the antibody comprises VH and/or VL domain framework regions of human germline gene segment sequences.

In one embodiment, the antibody comprises an antibody VH domain which
i) is derived from recombination of a human heavy chain V gene segment, a human heavy chain D gene segment and a human heavy chain J gene segment, wherein the V gene segment is IGHV4-4*02, IGHV3-7*01, IGHV3-9*01, IGHV3-30*18, IGHV1-46*03, IGHV3-33*01 or IGHV3-23*04; and/or the J gene segment is IGHJ6*02, IGHJ3*02 or IGHJ4*02; or
ii) comprises framework regions FR1, FR2, FR3 and FR4, wherein FR1 aligns with human germline V gene segment IGHV4-4*02, IGHV3-7*01, IGHV3-9*01, IGHV3-30*18, IGHV1-46*03, IGHV3-33*01 or IGHV3-23*04 with up to 1, 2, 3, 4, or 5 amino acid alterations, FR2 aligns with human germline V gene segment IGHV4-4*02, IGHV3-7*01, IGHV3-9*01, IGHV3-30*18, IGHV1-46*03, IGHV3-33*01 or IGHV3-23*04 with up to 1, 2, 3, 4, or 5 amino acid alterations, FR3 aligns with human germline V gene segment IGHV4-4*02, IGHV3-7*01, IGHV3-9*01, IGHV3-30*18, IGHV1-46*03, IGHV3-33*01 or IGHV3-23*04 with up to 1, 2, 3, 4, or 5 amino acid alterations, and/or FR4 aligns with human germline J gene segment IGHJ6*02, IGHJ3*02 or IGHJ4*02 with up to 1, 2, 3, 4 or 5 amino acid alterations.

In one embodiment, the antibody comprises an antibody VH domain which
i) is derived from recombination of a human heavy chain V gene segment IGHV4-4*02, IGHV3-7*01, IGHV3-9*01, IGHV3-30*18, IGHV1-46*03, IGHV3-33*01 or IGHV3-23*04, a human heavy chain D gene segment and a human heavy chain J gene segment, or
ii) comprises framework regions FR1, FR2, FR3 and FR4, wherein FR1, FR2 and FR3 each align with human germline V segment IGHV4-4*02, IGHV3-7*01, IGHV3-9*01, IGHV3-30*18, IGHV1-46*03, IGHV3-33*01 or IGHV3-23*04 with up to 1, 2, 3, 4 or 5 amino acid alterations.

In one embodiment, the J gene segment is IGHJ6*02, IGHJ3*02 or IGHJ4*02, or the VH domain framework region FR4 aligns with human germline J gene segment IGHJ6*02, IGHJ3*02 or IGHJ4*02 with 1, 2, 3, 4 or 5 amino acid alterations.

In one embodiment, the antibody comprises an antibody VL domain which
i) is derived from recombination of a human light chain V gene segment and a human light chain J gene segment, wherein
the V gene segment is IGKV3-20*01, IGKV1-16*02, IGKV1-33*01, IGKV2-30*01, IGKV2D-29*01, IGLV1-40*01, IGLV3-1*01, IGKV2D-28*d01, IGKV1-12*01 or IGLV1-51*01; and/or
the J gene segment is IGKJ1*01, IGKJ3*01, IGKJ4*01, IGLJ3*02, IGLJ2*01 or IGKJ5*01; or
ii) comprises framework regions FR1, FR2, FR3 and FR4, wherein
FR1 aligns with human germline V gene segment IGKV3-20*01, IGKV1-16*02, IGKV1-33*01, IGKV2-30*01, IGKV2D-29*01, IGLV1-40*01, IGLV3-1*01, IGKV2D-28*d01, IGKV1-12*01 or IGLV1-51*01 with up to 1, 2, 3, 4, or 5 amino acid alterations,
FR2 aligns with human germline V gene segment IGKV3-20*01, IGKV1-16*02, IGKV1-33*01, IGKV2-30*01, IGKV2D-29*01, IGLV1-40*01, IGLV3-1*01, IGKV2D-28*d01, IGKV1-12*01 or IGLV1-51*01 with up to 1, 2, 3, 4, or 5 amino acid alterations
FR3 aligns with human germline V gene segment IGKV3-20*01, IGKV1-16*02, IGKV1-33*01, IGKV2-30*01, IGKV2D-29*01, IGLV1-40*01, IGLV3-1*01, IGKV2D-28*d01, IGKV1-12*01 or IGLV1-51*01 with up to 1, 2, 3, 4 or 5 amino acid alterations, and/or
FR4 aligns with human germline J gene segment IGKJ1*01, IGKJ3*01, IGKJ4*01, IGLJ3*02, IGLJ2*01 or IGKJ5*01 with up to 1, 2, 3, 4 or 5 amino acid alterations.

In one embodiment, the antibody comprises an antibody VL domain derived from recombination of a human light chain V gene segment and a human light chain J gene segment, wherein:
the V gene segment is IGKV3-20*01, IGKV1-16*02, IGKV1-33*01, IGKV2-30*01, IGKV2D-29*01, IGLV1-40*01, IGLV3-1*01, IGKV2D-28*d01, IGKV1-12*01 or IGLV1-51*01, and optionally
the J gene segment is IGKJ1*01, IGKJ3*01, IGKJ4*01, IGLJ3*02, IGLJ2*01 or IGKJ5*01.

Example combinations of v and j gene segments for heavy and light chain variable domains are shown in Table 3, and these represent preferred combinations. The heavy and light chain variable domains may optionally be derived from the v and j gene segments identified in Table 3 for any one individual YANG antibody.

### Group D—Non-Competing RBD Binders

In a fourth aspect, the present invention provides an antibody that specifically binds to the receptor binding domain (RBD) of the SARS-CoV-2 spike protein, wherein the antibody does not compete for binding to the SARS-CoV2 spike protein with the human ACE2 receptor.

In one embodiment, the antibody is a neutralising antibody.

In one embodiment, the antibody neutralises SARS-CoV-2 with an IC50 of 55 nM or lower (e.g. as measured in a pseudovirus neutralisation assay).

In one embodiment, the antibody neutralises SARS-CoV-2 with an IC50 of 35 nM or lower (e.g. as measured in a pseudovirus neutralisation assay).

In one embodiment, the antibody neutralises SARS-CoV-2 with an IC50 of 15 nM or lower (e.g. as measured in a pseudovirus neutralisation assay).

In one embodiment, the antibody neutralises SARS-CoV-2 with an IC50 of 10 nM or lower (e.g. as measured in a pseudovirus neutralisation assay).

In one embodiment, the antibody neutralises SARS-CoV-2 with an IC50 of 3 nM or lower (e.g. as measured in a pseudovirus neutralisation assay).

In one embodiment, the antibody increases binding between SARS-CoV-2 and the human ACE2 receptor.

In one embodiment, the antibody comprises a variable heavy (VH) domain sequence comprising complementarity determining regions (CDRs) HCDR1, HCDR2 and HCDR3, and a variable light (VL) domain sequence comprising complementarity determining regions LCDR1, LCDR2 and LCDR3, wherein the HCDR3 is the HCDR3 of antibody IMPI-026, IMPI-034, IMPI-016, IMPI-050, IMPI-049, IMPI-015, IMPI-009, IMPI-011, IMPI-044, IMPI-046, IMPI-051, IMPI-024, IMPI-058, IMPI-043, IMPI-045, IMPI-027, IMPI-018, IMPI-048, IMPI-033, IMPI-014, IMPI-038 or IMPI-068.

In one embodiment, the antibody is a neutralising antibody and comprises a variable heavy (VH) domain sequence comprising complementarity determining regions (CDRs) HCDR1, HCDR2 and HCDR3, and a variable light (VL) domain sequence comprising complementarity determining regions LCDR1, LCDR2 and LCDR3, wherein the HCDR3 is the HCDR3 of antibody IMPI-024, IMPI-027, IMPI-038 or IMPI-068 or an antibody in the same cluster as one of these antibodies.

In one embodiment, the antibody is a neutralising antibody and comprises a variable heavy (VH) domain sequence comprising complementarity determining regions (CDRs) HCDR1, HCDR2 and HCDR3, and a variable light (VL) domain sequence comprising complementarity determining regions LCDR1, LCDR2 and LCDR3, wherein the HCDR3 is the HCDR3 of antibody IMPI-024, IMPI-027, IMPI-038 or IMPI-068.

In one embodiment, the antibody increases binding between SARS-CoV2 and the human ACE2 receptor and comprises a variable heavy (VH) domain sequence comprising complementarity determining regions (CDRs) HCDR1, HCDR2 and HCDR3, and a variable light (VL) domain sequence comprising complementarity determining regions LCDR1, LCDR2 and LCDR3, wherein the HCDR3 is the HCDR3 of antibody IMPI-027, IMPI-033, IMPI-038 or IMPI-068 or an antibody in the same cluster as one of these antibodies.

In one embodiment, the antibody increases binding between SARS-CoV2 and the human ACE2 receptor and comprises a variable heavy (VH) domain sequence comprising complementarity determining regions (CDRs) HCDR1, HCDR2 and HCDR3, and a variable light (VL) domain sequence comprising complementarity determining regions LCDR1, LCDR2 and LCDR3, wherein the HCDR3 is the HCDR3 of antibody IMPI-027, IMPI-033, IMPI-038 or IMPI-068.

In one embodiment, HCDR3 is the HCDR3 of antibody IMPI-026.

In one embodiment, HCDR3 is the HCDR3 of antibody IMPI-034.

In one embodiment, HCDR3 is the HCDR3 of antibody IMPI-016.

In one embodiment, HCDR3 is the HCDR3 of antibody IMPI-050.

In one embodiment, HCDR3 is the HCDR3 of antibody IMPI-049.

In one embodiment, HCDR3 is the HCDR3 of antibody IMPI-015.

In one embodiment, HCDR3 is the HCDR3 of antibody IMPI-009.

In one embodiment, HCDR3 is the HCDR3 of antibody IMPI-011.

In one embodiment, HCDR3 is the HCDR3 of antibody IMPI-044.

In one embodiment, HCDR3 is the HCDR3 of antibody IMPI-046.

In one embodiment, HCDR3 is the HCDR3 of antibody IMPI-051.

In one embodiment, HCDR3 is the HCDR3 of antibody IMPI-024.

In one embodiment, HCDR3 is the HCDR3 of antibody IMPI-058.

In one embodiment, HCDR3 is the HCDR3 of antibody IMPI-043.

In one embodiment, HCDR3 is the HCDR3 of antibody IMPI-045.

In one embodiment, HCDR3 is the HCDR3 of antibody IMPI-027.

In one embodiment, HCDR3 is the HCDR3 of antibody IMPI-018.

In one embodiment, HCDR3 is the HCDR3 of antibody IMPI-048.

In one embodiment, HCDR3 is the HCDR3 of antibody IMPI-033.

In one embodiment, HCDR3 is the HCDR3 of antibody IMPI-014.

In one embodiment, HCDR3 is the HCDR3 of antibody IMPI-038.

In one embodiment, HCDR3 is the HCDR3 of antibody IMPI-068.

In one embodiment, the present invention provides an anti-SARS-CoV-2 antibody, wherein the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3, wherein the CDRs are those of antibody IMPI-026, IMPI-034, IMPI-016, IMPI-050, IMPI-049, IMPI-015, IMPI-009, IMPI-011, IMPI-044, IMPI-046, IMPI-051, IMPI-024, IMPI-058, IMPI-043, IMPI-045, IMPI-027, IMPI-018, IMPI-048, IMPI-033, IMPI-014, IMPI-038 or IMPI-068.

In one embodiment, the antibody comprises a variable heavy (VH) domain sequence comprising complementarity determining regions (CDRs) HCDR1, HCDR2 and HCDR3, and a variable light (VL) domain sequence comprising complementarity determining regions LCDR1, LCDR2 and LCDR3, wherein the CDRs are those of antibody IMPI-026, IMPI-034, IMPI-016, IMPI-050, IMPI-049, IMPI-015, IMPI-009, IMPI-011, IMPI-044, IMPI-046, IMPI-051, IMPI-024, IMPI-058, IMPI-043, IMPI-045, IMPI-027, IMPI-018, IMPI-048, IMPI-033, IMPI-014, IMPI-038 or IMPI-068.

In one embodiment, the antibody is a neutralising antibody, wherein the antibody comprises a variable heavy (VH) domain sequence comprising complementarity determining regions (CDRs) HCDR1, HCDR2 and HCDR3, and a variable light (VL) domain sequence comprising complementarity determining regions LCDR1, LCDR2 and LCDR3, wherein the CDRs are those of antibody IMPI-024, IMPI-027, IMPI-038 or IMPI-068 or an antibody in the same cluster as one ofthese antibodies.

In one embodiment, the antibody is a neutralising antibody, wherein the antibody comprises a variable heavy (VH) domain sequence comprising complementarity determining regions (CDRs) HCDR1, HCDR2 and HCDR3, and a variable light (VL) domain sequence comprising complementarity determining regions LCDR1, LCDR2 and LCDR3, wherein the CDRs are those of antibody IMPI-024, IMPI-027, IMPI-038 or IMPI-068.

In one embodiment, the antibody increases binding between SARS-CoV-2 and the human ACE2 receptor, wherein the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3, wherein the CDRs are those of antibody IMPI-027, IMPI-033, IMPI-038 or IMPI-068, or an antibody in the same cluster as one ofthese antibodies.

In one embodiment, the antibody increases binding between SARS-CoV-2 and the human ACE2 receptor, wherein the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3, wherein the CDRs are those of antibody IMPI-027, IMPI-033, IMPI-038 or IMPI-068.

In one embodiment, the antibody has the CDRs of antibody IMPI-026.

In one embodiment, the antibody has the CDRs of antibody IMPI-034.

In one embodiment, the antibody has the CDRs of antibody IMPI-016.

In one embodiment, the antibody has the CDRs of antibody IMPI-050.

In one embodiment, the antibody has the CDRs of antibody IMPI-049.

In one embodiment, the antibody has the CDRs of antibody IMPI-015.

In one embodiment, the antibody has the CDRs of antibody IMPI-009.

In one embodiment, the antibody has the CDRs of antibody IMPI-011.

In one embodiment, the antibody has the CDRs of antibody IMPI-044.

In one embodiment, the antibody has the CDRs of antibody IMPI-046.

In one embodiment, the antibody has the CDRs of antibody IMPI-051.

In one embodiment, the antibody has the CDRs of antibody IMPI-024.

In one embodiment, the antibody has the CDRs of antibody IMPI-058.

In one embodiment, the antibody has the CDRs of antibody IMPI-043.

In one embodiment, the antibody has the CDRs of antibody IMPI-045.

In one embodiment, the antibody has the CDRs of antibody IMPI-027.

In one embodiment, the antibody has the CDRs of antibody IMPI-018.

In one embodiment, the antibody has the CDRs of antibody IMPI-048.

In one embodiment, the antibody has the CDRs of antibody IMPI-033.

In one embodiment, the antibody has the CDRs of antibody IMPI-014.

In one embodiment, the antibody has the CDRs of antibody IMPI-038.

In one embodiment, the antibody has the CDRs of antibody IMPI-068.

In one embodiment, the antibody comprises a variable heavy (VH) domain sequence and a variable light (VL) domain sequence and wherein the variable heavy (VH) domain and variable light (VL) domain sequences respectively comprise the variable heavy (VH) domain and variable light (VL) domain sequences of antibody IMPI-026, IMPI-034, IMPI-016, IMPI-050, IMPI-049, IMPI-015, IMPI-009, IMPI-011, IMPI-044, IMPI-046, IMPI-051, IMPI-024, IMPI-058, IMPI-043, IMPI-045, IMPI-027, IMPI-018, IMPI-048, IMPI-033, IMPI-014, IMPI-038 or IMPI-068, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

In one embodiment, the antibody is a neutralising antibody, wherein the antibody comprises a variable heavy (VH) domain sequence and a variable light (VL) domain sequence and wherein the variable heavy (VH) domain and variable light (VL) domain sequences respectively comprise the variable heavy (VH) domain and variable light (VL) domain sequences of antibody IMPI-024, IMPI-027, IMPI-038 or IMPI-068, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence or an antibody in the same cluster as one of these antibodies.

In one embodiment, the antibody is a neutralising antibody, wherein the antibody comprises a variable heavy (VH) domain sequence and a variable light (VL) domain sequence and wherein the variable heavy (VH) domain and variable light (VL) domain sequences respectively comprise the variable heavy (VH) domain and variable light (VL) domain sequences of antibody IMPI-024, IMPI-027, IMPI-038 or IMPI-068, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

In one embodiment, the antibody increases binding between SARS-CoV-2 and the human ACE2 receptor, wherein the antibody comprises a variable heavy (VH) domain sequence and a variable light (VL) domain sequence and wherein the variable heavy (VH) domain and variable light (VL) domain sequences respectively comprise the variable heavy (VH) domain and variable light (VL) domain sequences of antibody IMPI-027, IMPI-033, IMPI-038 or IMPI-068, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence or an antibody in the same cluster as one of these antibodies.

In one embodiment, the antibody increases binding between SARS-CoV-2 and the human ACE2 receptor, wherein the antibody comprises a variable heavy (VH) domain sequence and a variable light (VL) domain sequence and wherein the variable heavy (VH) domain and variable light (VL) domain sequences respectively comprise the variable heavy (VH) domain and variable light (VL) domain sequences of antibody IMPI-027, IMPI-033, IMPI-038 or IMPI-068, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

In one embodiment, the antibody comprises a variable heavy (VH) domain sequence of antibody IMPI-026, optionally with 1, 2, 3, 4 or 5 amino acid alterations outside the complementarity determining regions (CDRs), and a variable light (VL) domain sequence of antibody IMPI-026, optionally with 1, 2, 3, 4 or 5 amino acid alterations outside the complementarity determining regions (CDRs).

In one embodiment, the antibody comprises a variable heavy (VH) domain sequence of antibody IMPI-034, optionally with 1, 2, 3, 4 or 5 amino acid alterations outside the complementarity determining regions (CDRs), and a variable light (VL) domain sequence of antibody IMPI-034, optionally with 1, 2, 3, 4 or 5 amino acid alterations outside the complementarity determining regions (CDRs).

In one embodiment, the antibody comprises a variable heavy (VH) domain sequence of antibody IMPI-016, optionally with 1, 2, 3, 4 or 5 amino acid alterations outside the complementarity determining regions (CDRs), and a variable light (VL) domain sequence of antibody IMPI-016, optionally with 1, 2, 3, 4 or 5 amino acid alterations outside the complementarity determining regions (CDRs).

In one embodiment, the antibody comprises a variable heavy (VH) domain sequence of antibody IMPI-050, optionally with 1, 2, 3, 4 or 5 amino acid alterations outside the complementarity determining regions (CDRs), and a variable light (VL) domain sequence of antibody IMPI-050, optionally with 1, 2, 3, 4 or 5 amino acid alterations outside the complementarity determining regions (CDRs).

In one embodiment, the antibody comprises a variable heavy (VH) domain sequence of antibody IMPI-049, optionally with 1, 2, 3, 4 or 5 amino acid alterations outside the complementarity determining regions (CDRs), and a variable light (VL) domain sequence of antibody IMPI-049, optionally with 1, 2, 3, 4 or 5 amino acid alterations outside the complementarity determining regions (CDRs).

In one embodiment, the antibody comprises a variable heavy (VH) domain sequence of antibody IMPI-015, optionally with 1, 2, 3, 4 or 5 amino acid alterations outside the complementarity determining regions (CDRs), and a variable light (VL) domain sequence of antibody IMPI-015, optionally with 1, 2, 3, 4 or 5 amino acid alterations outside the complementarity determining regions (CDRs).

In one embodiment, the antibody comprises a variable heavy (VH) domain sequence of antibody IMPI-009, optionally with 1, 2, 3, 4 or 5 amino acid alterations outside the complementarity determining regions (CDRs), and a variable light (VL) domain sequence of antibody IMPI-009, optionally with 1, 2, 3, 4 or 5 amino acid alterations outside the complementarity determining regions (CDRs).

In one embodiment, the antibody comprises a variable heavy (VH) domain sequence of antibody IMPI-011, optionally with 1, 2, 3, 4 or 5 amino acid alterations outside the complementarity determining regions (CDRs), and a variable light (VL) domain sequence of antibody IMPI-011, optionally with 1, 2, 3, 4 or 5 amino acid alterations outside the complementarity determining regions (CDRs).

In one embodiment, the antibody comprises a variable heavy (VH) domain sequence of antibody IMPI-044, optionally with 1, 2, 3, 4 or 5 amino acid alterations outside the complementarity determining regions (CDRs), and a variable light (VL) domain sequence of antibody IMPI-044, optionally with 1, 2, 3, 4 or 5 amino acid alterations outside the complementarity determining regions (CDRs).

In one embodiment, the antibody comprises a variable heavy (VH) domain sequence of antibody IMPI-046, optionally with 1, 2, 3, 4 or 5 amino acid alterations outside the complementarity determining regions (CDRs), and a variable light (VL) domain sequence of antibody IMPI-046, optionally with 1, 2, 3, 4 or 5 amino acid alterations outside the complementarity determining regions (CDRs).

In one embodiment, the antibody comprises a variable heavy (VH) domain sequence of antibody IMPI-051, optionally with 1, 2, 3, 4 or 5 amino acid alterations outside the complementarity determining regions (CDRs), and a variable light (VL) domain sequence of antibody IMPI-051, optionally with 1, 2, 3, 4 or 5 amino acid alterations outside the complementarity determining regions (CDRs).

In one embodiment, the antibody comprises a variable heavy (VH) domain sequence of antibody IMPI-024, optionally with 1, 2, 3, 4 or 5 amino acid alterations outside the complementarity determining regions (CDRs), and a variable light (VL) domain sequence of antibody IMPI-024, optionally with 1, 2, 3, 4 or 5 amino acid alterations outside the complementarity determining regions (CDRs).

In one embodiment, the antibody comprises a variable heavy (VH) domain sequence of antibody IMPI-058, optionally with 1, 2, 3, 4 or 5 amino acid alterations outside the complementarity determining regions (CDRs), and a variable light (VL) domain sequence of antibody IMPI-058, optionally with 1, 2, 3, 4 or 5 amino acid alterations outside the complementarity determining regions (CDRs).

In one embodiment, the antibody comprises a variable heavy (VH) domain sequence of antibody IMPI-043, optionally with 1, 2, 3, 4 or 5 amino acid alterations outside the complementarity determining regions (CDRs), and a variable light (VL) domain sequence of antibody IMPI-043, optionally with 1, 2, 3, 4 or 5 amino acid alterations outside the complementarity determining regions (CDRs).

In one embodiment, the antibody comprises a variable heavy (VH) domain sequence of antibody IMPI-045, optionally with 1, 2, 3, 4 or 5 amino acid alterations outside the complementarity determining regions (CDRs), and a variable light (VL) domain sequence of antibody IMPI-045, optionally with 1, 2, 3, 4 or 5 amino acid alterations outside the complementarity determining regions (CDRs).

In one embodiment, the antibody comprises a variable heavy (VH) domain sequence of antibody IMPI-027, optionally with 1, 2, 3, 4 or 5 amino acid alterations outside the complementarity determining regions (CDRs), and a variable light (VL) domain sequence of antibody IMPI-027, optionally with 1, 2, 3, 4 or 5 amino acid alterations outside the complementarity determining regions (CDRs).

In one embodiment, the antibody comprises a variable heavy (VH) domain sequence of antibody IMPI-018, optionally with 1, 2, 3, 4 or 5 amino acid alterations outside the complementarity determining regions (CDRs), and a variable light (VL) domain sequence of antibody IMPI-018, optionally with 1, 2, 3, 4 or 5 amino acid alterations outside the complementarity determining regions (CDRs).

In one embodiment, the antibody comprises a variable heavy (VH) domain sequence of antibody IMPI-048, optionally with 1, 2, 3, 4 or 5 amino acid alterations outside the complementarity determining regions (CDRs), and a variable light (VL) domain sequence of antibody IMPI-048, optionally with 1, 2, 3, 4 or 5 amino acid alterations outside the complementarity determining regions (CDRs).

In one embodiment, the antibody comprises a variable heavy (VH) domain sequence of antibody IMPI-033, optionally with 1, 2, 3, 4 or 5 amino acid alterations outside the complementarity determining regions (CDRs), and a variable light (VL) domain sequence of antibody IMPI-033, optionally with 1, 2, 3, 4 or 5 amino acid alterations outside the complementarity determining regions (CDRs).

In one embodiment, the antibody comprises a variable heavy (VH) domain sequence of antibody IMPI-014, optionally with 1, 2, 3, 4 or 5 amino acid alterations outside the complementarity determining regions (CDRs), and a variable light (VL) domain sequence of antibody IMPI-014, optionally with 1, 2, 3, 4 or 5 amino acid alterations outside the complementarity determining regions (CDRs).

In one embodiment, the antibody comprises a variable heavy (VH) domain sequence of antibody IMPI-038, optionally with 1, 2, 3, 4 or 5 amino acid alterations outside the complementarity determining regions (CDRs), and a variable light (VL) domain sequence of antibody IMPI-038, optionally with 1, 2, 3, 4 or 5 amino acid alterations outside the complementarity determining regions (CDRs).

In one embodiment, the antibody comprises a variable heavy (VH) domain sequence of antibody IMPI-068, optionally with 1, 2, 3, 4 or 5 amino acid alterations outside the complementarity determining regions (CDRs), and a variable light (VL) domain sequence of antibody IMPI-068, optionally with 1, 2, 3, 4 or 5 amino acid alterations outside the complementarity determining regions (CDRs).

In one embodiment, the antibody comprises a variable heavy (VH) domain sequence and a variable light (VL) domain sequence and wherein the variable heavy (VH) domain and variable light (VL) domain sequences respectively comprise a sequence having at least 90% identity to the variable heavy (VH) and variable light (VL) domain sequences of antibody IMPI-026, IMPI-034, IMPI-016, IMPI-050, IMPI-049, IMPI-015, IMPI-009, IMPI-011, IMPI-044, IMPI-046, IMPI-051, IMPI-024, IMPI-058, IMPI-043, IMPI-045, IMPI-027, IMPI-018, IMPI-048, IMPI-033, IMPI-014, IMPI-038 or IMPI-068, provided that the antibody has the CDRs of antibody IMPI-026, IMPI-034, IMPI-016, IMPI-050, IMPI-049, IMPI-015, IMPI-009, IMPI-011, IMPI-044, IMPI-046, IMPI-051, IMPI-024, IMPI-058, IMPI-043, IMPI-045, IMPI-027, IMPI-018, IMPI-048, IMPI-033, IMPI-014, IMPI-038 or IMPI-068.

In one embodiment, the antibody is a neutralising antibody, wherein the antibody comprises a variable heavy (VH) domain sequence and a variable light (VL) domain sequence and wherein the variable heavy (VH) domain and variable light (VL) domain sequences respectively comprise a sequence having at least 90% identity to the variable heavy (VH) and variable light (VL) domain sequences of antibody IMPI-024, IMPI-027, IMPI-038 or IMPI-068, or an antibody in the same cluster as one of these antibodies, provided that the antibody has the CDRs of antibody IMPI-024, IMPI-027, IMPI-038 or IMPI-068, or an antibody in the same cluster as one ofthese antibodies.

In one embodiment, the antibody is a neutralising antibody, wherein the antibody comprises a variable heavy (VH) domain sequence and a variable light (VL) domain sequence and wherein the variable heavy (VH) domain and variable light (VL) domain sequences respectively comprise a sequence having at least 90% identity to the variable heavy (VH) and variable light (VL) domain sequences of antibody IMPI-024, IMPI-027, IMPI-038 or IMPI-068, provided that the antibody has the CDRs of antibody IMPI-024, IMPI-027, IMPI-038 or IMPI-068.

In one embodiment, the antibody increases binding between SARS-CoV2 and the human ACE2 receptor, wherein the antibody comprises a variable heavy (VH) domain sequence and a variable light (VL) domain sequence and wherein the variable heavy (VH) domain and variable light (VL) domain sequences respectively comprise a sequence having at least 90% identity to the variable heavy (VH) and variable light (VL) domain sequences of antibody IMPI-027, IMPI-033, IMPI-038 or IMPI-068, or an antibody in the same cluster as one of these antibodies, provided that the antibody has the CDRs of antibody IMPI-027, IMPI-033, IMPI-038 or IMPI-068, or an antibody in the same cluster as one of these antibodies.

In one embodiment, the antibody increases binding between SARS-CoV2 and the human ACE2 receptor, wherein the antibody comprises a variable heavy (VH) domain sequence and a variable light (VL) domain sequence and wherein the variable heavy (VH) domain and variable light (VL) domain sequences respectively comprise a sequence having at least 90% identity to the variable heavy (VH) and variable light (VL) domain sequences of antibody IMPI-027, IMPI-033, IMPI-038 or IMPI-068, provided that the antibody has the CDRs of antibody IMPI-027, IMPI-033, IMPI-038 or IMPI-068.

In one embodiment, the variable heavy (VH) domain sequence comprises a sequence having at least 90% identity to the VH domain sequence of antibody IMPI-026 and the variable light (VL) domain sequence comprises a sequence having at least 90% identity to the VL domain sequence of antibody IMPI-026, provided that the antibody has the CDRs of antibody IMPI-026.

In one embodiment, the variable heavy (VH) domain sequence comprises a sequence having at least 90% identity to the VH domain sequence of antibody IMPI-034 and the variable light (VL) domain sequence comprises a sequence having at least 90% identity to the VL domain sequence of antibody IMPI-034, provided that the antibody has the CDRs of antibody IMPI-034.

In one embodiment, the variable heavy (VH) domain sequence comprises a sequence having at least 90% identity to the VH domain sequence of antibody IMPI-016 and the variable light (VL) domain sequence comprises a sequence having at least 90% identity to the VL domain sequence of antibody IMPI-016, provided that the antibody has the CDRs of antibody IMPI-016.

In one embodiment, the variable heavy (VH) domain sequence comprises a sequence having at least 90% identity to the VH domain sequence of antibody IMPI-050 and the variable light (VL) domain sequence comprises a sequence having at least 90% identity to the VL domain sequence of antibody IMPI-050, provided that the antibody has the CDRs of antibody IMPI-050.

In one embodiment, the variable heavy (VH) domain sequence comprises a sequence having at least 90% identity to the VH domain sequence of antibody IMPI-049 and the variable light (VL) domain sequence comprises a sequence having at least 90% identity to the VL domain sequence of antibody IMPI-049, provided that the antibody has the CDRs of antibody IMPI-049.

In one embodiment, the variable heavy (VH) domain sequence comprises a sequence having at least 90% identity to the VH domain sequence of antibody IMPI-015 and the variable light (VL) domain sequence comprises a sequence having at least 90% identity to the VL domain sequence of antibody IMPI-015, provided that the antibody has the CDRs of antibody IMPI-015.

In one embodiment, the variable heavy (VH) domain sequence comprises a sequence having at least 90% identity to the VH domain sequence of antibody IMPI-009 and the variable light (VL) domain sequence comprises a sequence having at least 90% identity to the VL domain sequence of antibody IMPI-009, provided that the antibody has the CDRs of antibody IMPI-009.

In one embodiment, the variable heavy (VH) domain sequence comprises a sequence having at least 90% identity to the VH domain sequence of antibody IMPI-011 and the variable light (VL) domain sequence comprises a sequence having at least 90% identity to the VL domain sequence of antibody IMPI-011, provided that the antibody has the CDRs of antibody IMPI-011.

In one embodiment, the variable heavy (VH) domain sequence comprises a sequence having at least 90% identity to the VH domain sequence of antibody IMPI-044 and the variable light (VL) domain sequence comprises a sequence having at least 90% identity to the VL domain sequence of antibody IMPI-044, provided that the antibody has the CDRs of antibody IMPI-044.

In one embodiment, the variable heavy (VH) domain sequence comprises a sequence having at least 90% identity to the VH domain sequence of antibody IMPI-046 and the variable light (VL) domain sequence comprises a sequence having at least 90% identity to the VL domain sequence of antibody IMPI-046, provided that the antibody has the CDRs of antibody IMPI-046.

In one embodiment, the variable heavy (VH) domain sequence comprises a sequence having at least 90% identity to the VH domain sequence of antibody IMPI-051 and the variable light (VL) domain sequence comprises a sequence having at least 90% identity to the VL domain sequence of antibody IMPI-051, provided that the antibody has the CDRs of antibody IMPI-051.

In one embodiment, the variable heavy (VH) domain sequence comprises a sequence having at least 90% identity to the VH domain sequence of antibody IMPI-024 and the variable light (VL) domain sequence comprises a sequence having at least 90% identity to the VL domain sequence of antibody IMPI-024, provided that the antibody has the CDRs of antibody IMPI-024.

In one embodiment, the variable heavy (VH) domain sequence comprises a sequence having at least 90% identity to the VH domain sequence of antibody IMPI-058 and the variable light (VL) domain sequence comprises a sequence having at least 90% identity to the VL domain sequence of antibody IMPI-058, provided that the antibody has the CDRs of antibody IMPI-058.

In one embodiment, the variable heavy (VH) domain sequence comprises a sequence having at least 90% identity to the VH domain sequence of antibody IMPI-043 and the variable light (VL) domain sequence comprises a sequence having at least 90% identity to the VL domain sequence of antibody IMPI-043, provided that the antibody has the CDRs of antibody IMPI-043.

In one embodiment, the variable heavy (VH) domain sequence comprises a sequence having at least 90% identity to the VH domain sequence of antibody IMPI-045 and the variable light (VL) domain sequence comprises a sequence having at least 90% identity to the VL domain sequence of antibody IMPI-045, provided that the antibody has the CDRs of antibody IMPI-045.

In one embodiment, the variable heavy (VH) domain sequence comprises a sequence having at least 90% identity to the VH domain sequence of antibody IMPI-027 and the variable light (VL) domain sequence comprises a sequence having at least 90% identity to the VL domain sequence of antibody IMPI-027, provided that the antibody has the CDRs of antibody IMPI-027.

In one embodiment, the variable heavy (VH) domain sequence comprises a sequence having at least 90% identity to the VH domain sequence of antibody IMPI-018 and the variable light (VL) domain sequence comprises a sequence having at least 90% identity to the VL domain sequence of antibody IMPI-018, provided that the antibody has the CDRs of antibody IMPI-018.

In one embodiment, the variable heavy (VH) domain sequence comprises a sequence having at least 90% identity to the VH domain sequence of antibody IMPI-048 and the variable light (VL) domain sequence comprises a sequence having at least 90% identity to the VL domain sequence of antibody IMPI-048, provided that the antibody has the CDRs of antibody IMPI-048.

In one embodiment, the variable heavy (VH) domain sequence comprises a sequence having at least 90% identity to the VH domain sequence of antibody IMPI-033 and the variable light (VL) domain sequence comprises a sequence having at least 90% identity to the VL domain sequence of antibody IMPI-033, provided that the antibody has the CDRs of antibody IMPI-033.

In one embodiment, the variable heavy (VH) domain sequence comprises a sequence having at least 90% identity to the VH domain sequence of antibody IMPI-014 and the variable light (VL) domain sequence comprises a sequence having at least 90% identity to the VL domain sequence of antibody IMPI-014, provided that the antibody has the CDRs of antibody IMPI-014.

In one embodiment, the variable heavy (VH) domain sequence comprises a sequence having at least 90% identity to the VH domain sequence of antibody IMPI-038 and the variable light (VL) domain sequence comprises a sequence having at least 90% identity to the VL domain sequence of antibody IMPI-038, provided that the antibody has the CDRs of antibody IMPI-038.

In one embodiment, the variable heavy (VH) domain sequence comprises a sequence having at least 90% identity to the VH domain sequence of antibody IMPI-068 and the variable light (VL) domain sequence comprises a sequence having at least 90% identity to the VL domain sequence of antibody IMPI-068, provided that the antibody has the CDRs of antibody IMPI-068.

In one embodiment, the antibody comprises a variable heavy (VH) domain sequence and a variable light (VL) domain sequence and wherein the variable heavy (VH) domain and variable light (VL) domain sequences respectively comprise the variable heavy (VH) and variable light (VL) domain sequences of antibody IMPI-026, IMPI-034, IMPI-016, IMPI-050, IMPI-049, IMPI-015, IMPI-009, IMPI-011, IMPI-044, IMPI-046, IMPI-051, IMPI-024, IMPI-058, IMPI-043, IMPI-045, IMPI-027, IMPI-018, IMPI-048, IMPI-033, IMPI-014, IMPI-038 or IMPI-068.

In one embodiment, the antibody is a neutralising antibody, wherein the antibody comprises a variable heavy (VH) domain sequence and a variable light (VL) domain sequence and wherein the variable heavy (VH) domain and variable light (VL) domain sequences respectively comprise the variable heavy (VH) and variable light (VL) domain sequences of antibody IMPI-024, IMPI-027, IMPI-038 or IMPI-068 or an antibody in the same cluster as one of these antibodies.

In one embodiment, the antibody is a neutralising antibody, wherein the antibody comprises a variable heavy (VH) domain sequence and a variable light (VL) domain sequence and wherein the variable heavy (VH) domain and variable light (VL) domain sequences respectively comprise the variable heavy (VH) and variable light (VL) domain sequences of antibody IMPI-024, IMPI-027, IMPI-038 or IMPI-068.

In one embodiment, the antibody increases binding between SARS-CoV2 and the human ACE2 receptor, wherein the antibody comprises a variable heavy (VH) domain sequence and a variable light (VL) domain sequence and wherein the variable heavy (VH) domain and variable light (VL) domain sequences respectively comprise the variable heavy (VH) and variable light (VL) domain sequences of antibody IMPI-027, IMPI-033, IMPI-038 or IMPI-068 or an antibody in the same cluster as one of these antibodies.

In one embodiment, the antibody increases binding between SARS-CoV2 and the human ACE2 receptor, wherein the antibody comprises a variable heavy (VH) domain sequence and a variable light (VL) domain sequence and wherein the variable heavy (VH) domain and variable light (VL) domain sequences respectively comprise the variable heavy (VH) and variable light (VL) domain sequences of antibody IMPI-027, IMPI-033, IMPI-038 or IMPI-068.

In one embodiment, the present invention provides an anti-SARS-CoV2 antibody, wherein the antibody comprises a variable heavy (VH) domain sequence and a variable light (VL) domain sequence and wherein the variable heavy (VH) domain and variable light (VL) domain sequences respectively comprise the variable heavy (VH) and variable light (VL) domain sequences of antibody IMPI-026, IMPI-034, IMPI-016, IMPI-050, IMPI-049, IMPI-015, IMPI-009, IMPI-011, IMPI-044, IMPI-046, IMPI-051, IMPI-024, IMPI-058, IMPI-043, IMPI-045, IMPI-027, IMPI-018, IMPI-048, IMPI-033, IMPI-014, IMPI-038 or IMPI-068.

In one embodiment, the variable heavy (VH) domain sequence comprises the VH domain sequence of antibody IMPI-026 and the variable light (VL) domain sequence comprises the VL domain sequence of antibody IMPI-026.

In one embodiment, the variable heavy (VH) domain sequence comprises the VH domain sequence of antibody IMPI-034 and the variable light (VL) domain sequence comprises the VL domain sequence of antibody IMPI-034.

In one embodiment, the variable heavy (VH) domain sequence comprises the VH domain sequence of antibody IMPI-016 and the variable light (VL) domain sequence comprises the VL domain sequence of antibody IMPI-016.

In one embodiment, the variable heavy (VH) domain sequence comprises the VH domain sequence of antibody IMPI-050 and the variable light (VL) domain sequence comprises the VL domain sequence of antibody IMPI-050.

In one embodiment, the variable heavy (VH) domain sequence comprises the VH domain sequence of antibody IMPI-049 and the variable light (VL) domain sequence comprises the VL domain sequence of antibody IMPI-049.

In one embodiment, the variable heavy (VH) domain sequence comprises the VH domain sequence of antibody IMPI-015 and the variable light (VL) domain sequence comprises the VL domain sequence of antibody IMPI-015.

In one embodiment, the variable heavy (VH) domain sequence comprises the VH domain sequence of antibody IMPI-009 and the variable light (VL) domain sequence comprises the VL domain sequence of antibody IMPI-009.

In one embodiment, the variable heavy (VH) domain sequence comprises the VH domain sequence of antibody IMPI-011 and the variable light (VL) domain sequence comprises the VL domain sequence of antibody IMPI-011.

In one embodiment, the variable heavy (VH) domain sequence comprises the VH domain sequence of antibody IMPI-044 and the variable light (VL) domain sequence comprises the VL domain sequence of antibody IMPI-044.

In one embodiment, the variable heavy (VH) domain sequence comprises the VH domain sequence of antibody IMPI-046 and the variable light (VL) domain sequence comprises the VL domain sequence of antibody IMPI-046.

In one embodiment, the variable heavy (VH) domain sequence comprises the VH domain sequence of antibody IMPI-051 and the variable light (VL) domain sequence comprises the VL domain sequence of antibody IMPI-051.

In one embodiment, the variable heavy (VH) domain sequence comprises the VH domain sequence of antibody IMPI-024 and the variable light (VL) domain sequence comprises the VL domain sequence of antibody IMPI-024.

In one embodiment, the variable heavy (VH) domain sequence comprises the VH domain sequence of antibody IMPI-058 and the variable light (VL) domain sequence comprises the VL domain sequence of antibody IMPI-058.

In one embodiment, the variable heavy (VH) domain sequence comprises the VH domain sequence of antibody IMPI-043 and the variable light (VL) domain sequence comprises the VL domain sequence of antibody IMPI-043.

In one embodiment, the variable heavy (VH) domain sequence comprises the VH domain sequence of antibody IMPI-045 and the variable light (VL) domain sequence comprises the VL domain sequence of antibody IMPI-045.

In one embodiment, the variable heavy (VH) domain sequence comprises the VH domain sequence of antibody IMPI-027 and the variable light (VL) domain sequence comprises the VL domain sequence of antibody IMPI-027.

In one embodiment, the variable heavy (VH) domain sequence comprises the VH domain sequence of antibody IMPI-018 and the variable light (VL) domain sequence comprises the VL domain sequence of antibody IMPI-018.

In one embodiment, the variable heavy (VH) domain sequence comprises the VH domain sequence of antibody IMPI-048 and the variable light (VL) domain sequence comprises the VL domain sequence of antibody IMPI-048.

In one embodiment, the variable heavy (VH) domain sequence comprises the VH domain sequence of antibody IMPI-033 and the variable light (VL) domain sequence comprises the VL domain sequence of antibody IMPI-033.

In one embodiment, the variable heavy (VH) domain sequence comprises the VH domain sequence of antibody IMPI-014 and the variable light (VL) domain sequence comprises the VL domain sequence of antibody IMPI-014.

In one embodiment, the variable heavy (VH) domain sequence comprises the VH domain sequence of antibody IMPI-038 and the variable light (VL) domain sequence comprises the VL domain sequence of antibody IMPI-038.

In one embodiment, the variable heavy (VH) domain sequence comprises the VH domain sequence of antibody IMPI-068 and the variable light (VL) domain sequence comprises the VL domain sequence of antibody IMPI-068.

In one embodiment, the antibody neutralises SARS-CoV2 with an IC50 of 2 nM or greater (e.g. as measured in a pseudovirus neutralisation assay) (see, for example, IMPI-024; IMPI-068; IMPI-027; and IMPI-038).

In one embodiment, the antibody neutralises SARS-CoV2 with an IC50 of 5 nM or greater (e.g. as measured in a pseudovirus neutralisation assay) (see, for example, IMPI-024; IMPI-068; IMPI-027; IMPI-038).

In one embodiment, the antibody neutralises SARS-CoV2 with an IC50 of 10 nM or greater (e.g. as measured in a pseudovirus neutralisation assay) (see, for example, IMPI-068; IMPI-027; and IMPI-038).

In one embodiment, the antibody neutralises SARS-CoV2 with an IC50 of 30 nM or greater (e.g. as measured in a pseudovirus neutralisation assay) (see, for example, IMPI-027 and IMPI-038).

In one embodiment, the antibody neutralises SARS-CoV2 with an IC50 of 50 nM or greater (e.g. as measured in a pseudovirus neutralisation assay) (see, for example, IMPI-038).

In one embodiment, the antibody comprises VH and/or VL domain framework regions of human germline gene segment sequences.

In one embodiment, the antibody comprises an antibody VH domain which
i) is derived from recombination of a human heavy chain V gene segment, a human heavy chain D gene segment and a human heavy chain J gene segment, wherein the V gene segment is IGHV5-51*01, IGHV4-31*03 or IGHV3-30*18;
and/or the J gene segment is IGHJ4*02 or IGHJ6*02, or
ii) comprises framework regions FR1, FR2, FR3 and FR4, wherein FR1 aligns with human germline V gene segment IGHV5-51*01, IGHV4-31*03 or IGHV3-30*18 with up to 1, 2, 3, 4, or 5 amino acid alterations, FR2 aligns with human germline V gene segment IGHV5-51*01, IGHV4-31*03 or IGHV3-30*18 with up to 1, 2, 3, 4, or 5 amino acid alterations, FR3 aligns with human germline V gene segment IGHV5-51*01, IGHV4-31*03 or IGHV3-30*18 with up to 1, 2, 3, 4 or 5 amino acid alterations, and/or FR4 aligns with human germline J gene segment IGHJ4*02 or IGHJ6*02 with up to 1, 2, 3, 4 or 5 amino acid alterations.

In one embodiment, the antibody comprises an antibody VH domain which
i) is derived from recombination of a human heavy chain V gene segment IGHV5-51*01, IGHV4-31*03 or IGHV3-30*18, a human heavy chain D gene segment and a human heavy chain J gene segment, or
ii) comprises framework regions FR1, FR2, FR3 and FR4, wherein FR1, FR2 and FR3 each align with human germline V segment IGHV5-51*01, IGHV4-31*03 or IGHV3-30*18 with up to 1, 2, 3, 4 or 5 amino acid alterations.

In one embodiment, the J gene segment is IGHJ4*02 or IGHJ6*02, or the VH domain framework region FR4 aligns with human germline J gene segment IGHJ4*02 or IGHJ6*02 with 1, 2, 3, 4 or 5 amino acid alterations.

In one embodiment, the antibody comprises an antibody VL domain which
i) is derived from recombination of a human light chain V gene segment and a human light chain J gene segment, wherein
   the V gene segment is IGKV1D-13*d01 or IGKV1-12*01, and/or
   the J gene segment is IGKJ1*01, IGKJ4*01 or IGKJ3*01; or
ii) comprises framework regions FR1, FR2, FR3 and FR4, wherein
   FR1 aligns with human germline V gene segment IGKV1D-13*d01 or IGKV1-12*01 with up to 1, 2, 3, 4, or 5 amino acid alterations,
   FR2 aligns with human germline V gene segment IGKV1D-13*d01 or IGKV1-12*01 with up to 1, 2, 3, 4, or 5 amino acid alterations
   FR3 aligns with human germline V gene segment IGKV1D-13*d01 or IGKV1-12*01 with up to 1, 2, 3, 4 or 5 amino acid alterations, and/or
   FR4 aligns with human germline J gene segment IGKJ1*01, IGKJ4*01 or IGKJ3*01 with up to 1, 2, 3, 4 or 5 amino acid alterations.

In one embodiment, the antibody comprises an antibody VL domain derived from recombination of a human light chain V gene segment and a human light chain J gene segment, wherein:
the V gene segment is IGKV1D-13*d01 or IGKV1-12*01, and optionally the J gene segment is IGKJ1*01, IGKJ4*01 or IGKJ3*01.

Example combinations of v and j gene segments for heavy and light chain variable domains are shown in Table 3, and these represent preferred combinations. The heavy and light chain variable domains may optionally be derived from the v and j gene segments identified in Table 3 for any one individual IMPI antibody identified in this Group D section.

Further non-competing RBD binders

According to the fourth aspect of the invention, further antibodies are provided herein which specifically bind to the RBD of the SARS-CoV-2 spike protein and do not compete for binding to the SARS-CoV-2 spike protein with the human ACE2 receptor. For example, antibodies YANG-1111, YANG-1102, YANG-1401, YANG-1401a, YANG-1401b, YANG-1401c, YANG-1401d, YANG-1401e, YANG-1402, YANG-1403, and YANG-2112 as exemplified herein have been found to specifically bind to the RBD of the SARS-CoV-2 spike protein and do not compete for binding to the SARS-CoV-2 spike protein with the human ACE2 receptor.

In one embodiment, the antibody comprises a variable heavy (VH) domain sequence comprising complementarity determining regions (CDRs) HCDR1, HCDR2 and HCDR3, and a variable light (VL) domain sequence comprising complementarity determining regions LCDR1, LCDR2 and LCDR3, wherein the HCDR3 is the HCDR3 of antibody YANG-1111, YANG-1102, YANG-1401, YANG-1401a, YANG-1401b, YANG-1401c, YANG-1401d, YANG-1401e, YANG-1402, YANG-1403 or YANG-2112.

In one embodiment, HCDR3 is the HCDR3 of antibody YANG-1102.

In one embodiment, HCDR3 is the HCDR3 of antibody YANG-1111.

In one embodiment, HCDR3 is the HCDR3 of antibody YANG-1401.

In one embodiment, HCDR3 is the HCDR3 of antibody YANG-1402.

In one embodiment, HCDR3 is the HCDR3 of antibody YANG-1403.

In one embodiment, HCDR3 is the HCDR3 of antibody YANG-2112.

In one embodiment, the antibody specifically binds to the RBD of the SARS-CoV-2 spike protein, and HCDR3 is the HCDR3 of antibody YANG-1111, YANG-1102, YANG-1401, YANG-1401a, YANG-1401b, YANG-1401c, YANG-1401d, YANG-1401e, YANG-1402, YANG-1403 or YANG-2112.

In one embodiment, the antibody specifically binds to the RBD of the SARS-CoV-2 spike protein with a KD of 0.1 nM or lower (e.g. as measured by surface plasmon resonance (SPR)), and HCDR3 is the HCDR3 of antibody YANG-1111, YANG-1102, YANG-1401, YANG-1401a, YANG-1401b, YANG-1401c, YANG-1401d, YANG-1401e, YANG-1402, YANG-1403 or YANG-2112.

In one embodiment, the antibody specifically binds to the RBD of the SARS-CoV-2 spike protein and neutralises SARS-CoV-2 with at least 70%, 85%, or 90% neutralisation (e.g. as measured in a pseudovirus neutralisation assay), and HCDR3 is the HCDR3 of antibody YANG-1111, YANG-1102, YANG-1401, YANG-1401a, YANG-1401b, YANG-1401c, YANG-1401d, YANG-1401e, YANG-1402, YANG-1403 or YANG-2112.

In one embodiment, the antibody specifically binds to the RBD of the SARS-CoV-2 spike protein with a KD of 0.1 nM or lower (e.g. as measured by surface plasmon resonance (SPR)) and neutralises SARS-CoV-2 with at least 70%, 85%, or 90% neutralisation (e.g. as measured in a pseudovirus neutralisation assay), and HCDR3 is the HCDR3 of antibody YANG-1I11, YANG-1102, YANG-1401, YANG-1401a, YANG-1401b, YANG-1401c, YANG-1401d, YANG-1401e, YANG-1402, YANG-1403 or YANG-2112.

In one embodiment, the present invention provides an anti-SARS-CoV-2 antibody, wherein the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3, wherein the CDRs are those of antibody YANG-1111, YANG-1102, YANG-1401, YANG-1401a, YANG-1401b, YANG-1401c, YANG-1401d, YANG-1401e, YANG-1402, YANG-1403 or YANG-2112.

In one embodiment, the antibody has the CDRs of antibody YANG-1102.

In one embodiment, the antibody has the CDRs of antibody YANG-1111.

In one embodiment, the antibody has the CDRs of antibody YANG-1401.

In one embodiment, the antibody has the CDRs of antibody YANG-1402.

In one embodiment, the antibody has the CDRs of antibody YANG-1403.

In one embodiment, the antibody has the CDRs of antibody YANG-2112.

In one embodiment, the antibody specifically binds to the RBD ofthe SARS-CoV-2 spike protein, and the CDRs are those of antibody YANG-1111, YANG-1102, YANG-1401, YANG-1401a, YANG-1401b, YANG-1401c, YANG-1401d, YANG-1401e, YANG-1402, YANG-1403 or YANG-2112.

In one embodiment, the antibody specifically binds to the RBD of the SARS-CoV-2 spike protein with a KD of 0.1 nM or lower (e.g. as measured by surface plasmon resonance (SPR)), and the CDRs are those of antibody YANG-1111, YANG-1102, YANG-1401, YANG-1401a, YANG-1401b, YANG-1401c, YANG-1401d, YANG-1401e, YANG-1402, YANG-1403 or YANG-2112.

In one embodiment, the antibody specifically binds to the RBD of the SARS-CoV-2 spike protein and neutralises SARS-CoV-2 with at least 70%, 85%, or 90% neutralisation (e.g. as measured in a pseudovirus neutralisation assay), and the CDRs are those of antibody YANG-1111, YANG-1102, YANG-1401, YANG-1401a, YANG-1401b, YANG-1401c, YANG-1401d, YANG-1401e, YANG-1402, YANG-1403 or YANG-2112.

In one embodiment, the antibody specifically binds to the RBD of the SARS-CoV-2 spike protein with a KD of 0.1 nM or lower (e.g. as measured by surface plasmon resonance (SPR)) and neutralises SARS-CoV-2 with at least 70%, 85%, or 90% neutralisation (e.g. as measured in a pseudovirus neutralisation assay), and the CDRs are those of antibody YANG-1111, YANG-1102, YANG-1401, YANG-1401a, YANG-1401b, YANG-1401c, YANG-1401d, YANG-1401e, YANG-1402, YANG-1403 or YANG-2112.

In one embodiment, the antibody comprises a variable heavy (VH) domain sequence and a variable light (VL) domain sequence and wherein the variable heavy (VH) domain and variable light (VL) domain sequences respectively comprise the variable heavy (VH) domain and variable light (VL) domain sequences of antibody YANG-1111, YANG-1102, YANG-1401, YANG-1401a, YANG-1401b, YANG-1401c, YANG-1401d, YANG-1401e, YANG-1402, YANG-1403 or YANG-2112, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

In one embodiment, the antibody comprises a variable heavy (VH) domain sequence of antibody YANG-1111, optionally with 1, 2, 3, 4 or 5 amino acid alterations outside the complementarity determining regions (CDRs), and a variable light (VL) domain sequence of antibody YANG-1111, optionally with 1, 2, 3, 4 or 5 amino acid alterations outside the complementarity determining regions (CDRs).

In one embodiment, the antibody comprises a variable heavy (VH) domain sequence of antibody YANG-1102, optionally with 1, 2, 3, 4 or 5 amino acid alterations outside the complementarity determining regions (CDRs), and a variable light (VL) domain sequence of antibody YANG-1102, optionally with 1, 2, 3, 4 or 5 amino acid alterations outside the complementarity determining regions (CDRs).

In one embodiment, the antibody comprises a variable heavy (VH) domain sequence of antibody YANG-1401, optionally with 1, 2, 3, 4 or 5 amino acid alterations outside the complementarity determining regions (CDRs), and a variable light (VL) domain sequence of antibody YANG-1401, optionally with 1, 2, 3, 4 or 5 amino acid alterations outside the complementarity determining regions (CDRs).

In one embodiment, the antibody comprises a variable heavy (VH) domain sequence of antibody YANG-1402, optionally with 1, 2, 3, 4 or 5 amino acid alterations outside the complementarity determining regions (CDRs), and a variable light (VL) domain sequence of antibody YANG-1402, optionally with 1, 2, 3, 4 or 5 amino acid alterations outside the complementarity determining regions (CDRs).

In one embodiment, the antibody comprises a variable heavy (VH) domain sequence of antibody YANG-1403, optionally with 1, 2, 3, 4 or 5 amino acid alterations outside the complementarity determining regions (CDRs), and a variable light (VL) domain sequence of antibody YANG-1403, optionally with 1, 2, 3, 4 or 5 amino acid alterations outside the complementarity determining regions (CDRs).

In one embodiment, the antibody comprises a variable heavy (VH) domain sequence of antibody YANG-2112, optionally with 1, 2, 3, 4 or 5 amino acid alterations outside the complementarity determining regions (CDRs), and a variable light (VL) domain sequence of antibody YANG-2112, optionally with 1, 2, 3, 4 or 5 amino acid alterations outside the complementarity determining regions (CDRs).

In one embodiment, the antibody specifically binds to the RBD of the SARS-CoV-2 spike protein, and the variable heavy (VH) domain and variable light (VL) domain sequences respectively comprise the variable heavy (VH) domain and variable light (VL) domain sequences of antibody YANG-1111, YANG-1102, YANG-1401, YANG-1401a, YANG-1401b, YANG-1401c, YANG-1401d, YANG-1401e, YANG-1402, YANG-1403 or YANG-2112, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

In one embodiment, the antibody specifically binds to the RBD of the SARS-CoV-2 spike protein with a KD of 0.1 nM or lower (e.g. as measured by surface plasmon resonance (SPR)), and the variable heavy (VH) domain and variable light (VL) domain sequences respectively comprise the variable heavy (VH) domain and variable light (VL) domain sequences of antibody YANG-1111, YANG-1102, YANG-1401, YANG-1401a, YANG-1401b, YANG-1401c, YANG-1401d, YANG-1401e, YANG-1402, YANG-1403 or YANG-2112, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

In one embodiment, the antibody specifically binds to the RBD of the SARS-CoV-2 spike protein and neutralises SARS-CoV-2 with at least 70%, 85%, or 90% neutralisation (e.g. as measured in a pseudovirus neutralisation assay), and the variable heavy (VH) domain and variable light (VL) domain sequences respectively comprise the variable heavy (VH) domain and variable light (VL) domain sequences of antibody YANG-1111, YANG-1102, YANG-1401, YANG-1401a, YANG-1401b, YANG-1401c, YANG-1401d, YANG-1401e, YANG-1402, YANG-1403 or YANG-2112, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

In one embodiment, the antibody specifically binds to the RBD of the SARS-CoV-2 spike protein with a KD of 0.1 nM or lower (e.g. as measured by surface plasmon resonance (SPR)) and neutralises SARS-CoV-2 with at least 70%, 85%, or 90% neutralisation (e.g. as measured in a pseudovirus neutralisation assay), and the variable heavy (VH) domain and variable light (VL) domain sequences respectively comprise the variable heavy (VH) domain and variable light (VL) domain sequences of antibody YANG-1111, YANG-1102, YANG-1401, YANG-1401a, YANG-1401b, YANG-1401c, YANG-1401d, YANG-1401e, YANG-1402, YANG-1403 or YANG-2112, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

In one embodiment, the antibody comprises a variable heavy (VH) domain sequence and a variable light (VL) domain sequence and wherein the variable heavy (VH) domain and variable light (VL) domain sequences respectively comprise a sequence having at least 90% identity to the variable heavy (VH) and variable light (VL) domain sequences of antibody provided that the antibody has the CDRs of antibody YANG-1111, YANG-1102, YANG-1401, YANG-1401a, YANG-1401b, YANG-1401c, YANG-1401d, YANG-1401e, YANG-1402, YANG-1403 or YANG-2112, respectively.

In one embodiment, the variable heavy (VH) domain sequence comprises a sequence having at least 90% identity to the VH domain sequence of antibody YANG-1102 and the variable light (VL) domain sequence comprises a sequence having at least 90% identity to the VL domain sequence of antibody YANG-1102, provided that the antibody has the CDRs of antibody YANG-1102.

In one embodiment, the variable heavy (VH) domain sequence comprises a sequence having at least 90% identity to the VH domain sequence of antibody YANG-1111 and the variable light (VL) domain sequence comprises a sequence having at least 90% identity to the VL domain sequence of antibody YANG-1111, provided that the antibody has the CDRs of antibody YANG-1111.

In one embodiment, the variable heavy (VH) domain sequence comprises a sequence having at least 90% identity to the VH domain sequence of antibody YANG-1401 and the variable light (VL) domain sequence comprises a sequence having at least 90% identity to the VL domain sequence of antibody YANG-1401, provided that the antibody has the CDRs of antibody YANG-1401.

In one embodiment, the variable heavy (VH) domain sequence comprises a sequence having at least 90% identity to the VH domain sequence of antibody YANG-1402 and the variable light (VL) domain sequence comprises a sequence having at least 90% identity to the VL domain sequence of antibody YANG-1402, provided that the antibody has the CDRs of antibody YANG-1402.

In one embodiment, the variable heavy (VH) domain sequence comprises a sequence having at least 90% identity to the VH domain sequence of antibody YANG-1403 and the variable light (VL) domain sequence comprises a sequence having at least 90% identity to the VL domain sequence of antibody YANG-1403, provided that the antibody has the CDRs of antibody YANG-1403.

In one embodiment, the variable heavy (VH) domain sequence comprises a sequence having at least 90% identity to the VH domain sequence of antibody YANG-2112 and the variable light (VL) domain sequence comprises a sequence having at least 90% identity to the VL domain sequence of antibody YANG-2112, provided that the antibody has the CDRs of antibody YANG-2112.

In one embodiment, the antibody specifically binds to the RBD of the SARS-CoV-2 spike protein, and the antibody comprises a variable heavy (VH) domain sequence and a variable light (VL) domain sequence and wherein the variable heavy (VH) domain and variable light (VL) domain sequences respectively comprise a sequence having at least 90% identity to the variable heavy (VH) and variable light (VL) domain sequences of antibody YANG-1111, YANG-1102, YANG-1401, YANG-1401a, YANG-1401b, YANG-1401c, YANG-1401d, YANG-1401e, YANG-1402, YANG-1403 or YANG-2112 provided that the antibody has the CDRs of YANG-1111, YANG-1102, YANG-1401, YANG-1401a, YANG-1401b, YANG-1401c, YANG-1401d, YANG-1401e, YANG-1402, YANG-1403 orYANG-2112, respectively.

In one embodiment, the antibody specifically binds to the RBD of the SARS-CoV-2 spike protein with a KD of 0.1 nM or lower (e.g. as measured by surface plasmon resonance (SPR)), and the antibody comprises a variable heavy (VH) domain sequence and a variable light (VL) domain sequence and wherein the variable heavy (VH) domain and variable light (VL) domain sequences respectively comprise a sequence having at least 90% identity to the variable heavy (VH) and variable light (VL) domain sequences of antibody YANG-1111, YANG-1102, YANG-1401, YANG-1401a, YANG-1401b, YANG-1401c, YANG-1401d, YANG-1401e, YANG-1402, YANG-1403 or YANG-2112 provided that the antibody has the CDRs of antibody YANG-1111, YANG-1102, YANG-1401, YANG-1401a, YANG-1401b, YANG-1401c, YANG-1401d, YANG-1401e, YANG-1402, YANG-1403 or YANG-2112, respectively.

In one embodiment, the antibody specifically binds to the RBD of the SARS-CoV-2 spike protein and neutralises SARS-CoV-2 with at least 70%, 85%, or 90% neutralisation (e.g. as measured in a pseudovirus neutralisation assay), and

the antibody comprises a variable heavy (VH) domain sequence and a variable light (VL) domain sequence and wherein the variable heavy (VH) domain and variable light (VL) domain sequences respectively comprise a sequence having at least 90% identity to the variable heavy (VH) and variable light (VL) domain sequences of antibody YANG-1111, YANG-1102, YANG-1401, YANG-1401a, YANG-1401b, YANG-1401c, YANG-1401d, YANG-1401e, YANG-1402, YANG-1403 or YANG-2112 provided that the antibody has the CDRs of antibody YANG-1111, YANG-1102, YANG-1401, YANG-1401a, YANG-1401b, YANG-1401c, YANG-1401d, YANG-1401e, YANG-1402, YANG-1403 or YANG-2112, respectively.

In one embodiment, the antibody specifically binds to the RBD of the SARS-CoV-2 spike protein with a KD of 0.1 nM or lower (e.g. as measured by surface plasmon resonance (SPR)) and neutralises SARS-CoV-2 with at least 70%, 85%, or 90% neutralisation (e.g. as measured in a pseudovirus neutralisation assay), and

the antibody comprises a variable heavy (VH) domain sequence and a variable light (VL) domain sequence and wherein the variable heavy (VH) domain and variable light (VL) domain sequences respectively comprise a sequence having at least 90% identity to the variable heavy (VH) and variable light (VL) domain sequences of antibody YANG-1111, YANG-1102, YANG-1401, YANG-1401a, YANG-1401b, YANG-1401c, YANG-1401d, YANG-1401e, YANG-1402, YANG-1403 or YANG-2112 provided that the antibody has the CDRs of antibody YANG-1111, YANG-1102, YANG-1401, YANG-1401a, YANG-1401b, YANG-1401c, YANG-1401d, YANG-1401e, YANG-1402, YANG-1403 or YANG-2112, respectively.

In one embodiment, the present invention provides an antibody specifically binds to the RBD of the SARS-CoV-2 spike protein,

wherein the antibody comprises a variable heavy (VH) domain sequence and a variable light (VL) domain sequence and wherein the variable heavy (VH) domain and variable light (VL) domain sequences respectively comprise the variable heavy (VH) and variable light (VL) domain sequences of antibody YANG-1111, YANG-1102, YANG-1401, YANG-1401a, YANG-1401b, YANG-1401c, YANG-1401d, YANG-1401e, YANG-1402, YANG-1403 or YANG-2112.

An antibody that specifically binds to the receptor binding domain (RBD) of the SARS-CoV-2 spike protein, wherein the antibody comprises the VH and VL domain sequences of antibody YANG-1102.

An antibody that specifically binds to the receptor binding domain (RBD) of the SARS-CoV-2 spike protein, wherein the antibody comprises the VH and VL domain sequences of antibody YANG-1111.

An antibody that specifically binds to the receptor binding domain (RBD) of the SARS-CoV-2 spike protein, wherein the antibody comprises the VH and VL domain sequences of antibody YANG-1401.

An antibody that specifically binds to the receptor binding domain (RBD) of the SARS-CoV-2 spike protein, wherein the antibody comprises the VH and VL domain sequences of antibody YANG-1402.

An antibody that specifically binds to the receptor binding domain (RBD) of the SARS-CoV-2 spike protein, wherein the antibody comprises the VH and VL domain sequences of antibody YANG-1403.

An antibody that specifically binds to the receptor binding domain (RBD) of the SARS-CoV-2 spike protein, wherein the antibody comprises the VH and VL domain sequences of antibody YANG-2112.

In one embodiment, the antibody comprises a variable heavy (VH) domain sequence and a variable light (VL) domain sequence and wherein the variable heavy (VH) domain and variable light (VL) domain sequences respectively comprise the variable heavy (VH) and variable light (VL) domain sequences of antibody YANG-1111, YANG-1102, YANG-1401, YANG-1401a, YANG-1401b, YANG-1401c, YANG-1401d, YANG-1401e, YANG-1402, YANG-1403 or YANG-2112.

In one embodiment, the antibody comprises the VH and VL domain sequences of antibody YANG-1102.

In one embodiment, the antibody comprises the VH and VL domain sequences of antibody YANG-1111.

In one embodiment, the antibody comprises the VH and VL domain sequences of antibody YANG-1401.

In one embodiment, the antibody comprises the VH and VL domain sequences of antibody YANG-1402.

In one embodiment, the antibody comprises the VH and VL domain sequences of antibody YANG-1403.

In one embodiment, the antibody comprises the VH and VL domain sequences of antibody YANG-2112.

Antibodies are provided which bind to the same epitope on the RBD of the SARS-CoV-2 spike protein as an antibody described anywhere herein.

An antibody is provided which bind to the same epitope as antibody YANG-1111, YANG-1102, YANG-1401, YANG-1401a, YANG-1401b, YANG-1401c, YANG-1401d, YANG-1401e, YANG-1402, YANG-1403 or YANG-2112, e.g. as defined by its VH and VL sequences.

An antibody is provided which bind to the same epitope as antibody YANG-1102.

An antibody is provided which bind to the same epitope as antibody YANG-1111.

An antibody is provided which bind to the same epitope as antibody YANG-1401.

An antibody is provided which bind to the same epitope as antibody YANG-1402.

An antibody is provided which bind to the same epitope as antibody YANG-1403.

An antibody is provided which bind to the same epitope as antibody YANG-2112.

An antibody may contact the SARS-CoV-2 spike protein with a footprint that fully or partly overlaps with that of an antibody disclosed anywhere herein. As described elsewhere herein, competition between antibodies may be determined, for example using SPR, and antibodies are provided which compete for binding to the spike protein (compete for binding to their epitope) with an IgG antibody as described anywhere herein.

An antibody of the present invention may be one which competes for binding to SARS-CoV-2 spike protein with any anti-RBD antibody described herein, such as YANG-1111, YANG-1102, YANG-1401, YANG-1401a, YANG-1401b, YANG-1401c, YANG-1401d, YANG-1401e, YANG-1402, YANG-1403 or YANG-2112, e.g. as defined by its VH and VL sequences.

An antibody of the present invention may be one which competes for binding to the SARS-CoV-2 spike protein with antibody YANG-1111.

An antibody of the present invention may be one which competes for binding to the SARS-CoV-2 spike protein with antibody YANG-1102.

An antibody of the present invention may be one which competes for binding to the SARS-CoV-2 spike protein with antibody YANG-1401.

An antibody of the present invention may be one which competes for binding to the SARS-CoV-2 spike protein with antibody YANG-1402.

An antibody of the present invention may be one which competes for binding to the SARS-CoV-2 spike protein with antibody YANG-1403.

An antibody of the present invention may be one which competes for binding to the SARS-CoV-2 spike protein with antibody YANG-2112.

In one embodiment, the antibody comprises an antibody VH domain which
i) is derived from recombination of a human heavy chain V gene segment, a human heavy chain D gene segment and a human heavy chain J gene segment, wherein the V gene segment is IGHV4-4*02, IGHV3-48*02, IGHV3-53*01, IGHV3-33*01 or IGHV1-24*d01; and/or the J gene segment is IGHJ4*02, IGHJ5*02 or IGHJ6*02; or
ii) comprises framework regions FR1, FR2, FR3 and FR4, wherein FR1 aligns with human germline V gene segment IGHV4-4*02, IGHV3-48*02, IGHV3-53*01, IGHV3-33*01 or IGHV1-24*d01 with up to 1, 2, 3, 4, or 5 amino acid alterations, FR2 aligns with human germline V gene segment IGHV4-4*02, IGHV3-48*02, IGHV3-53*01, IGHV3-33*01 or IGHV1-24*d01 with up to 1, 2, 3, 4, or 5 amino acid alterations, FR3 aligns with human germline V gene segment IGHV4-4*02, IGHV3-48*02, IGHV3-53*01, IGHV3-33*01 or IGHV1-24*d01 with up to 1, 2, 3, 4, or 5 amino acid alterations, and/or FR4 aligns with human germline J gene segment IGHJ4*02, IGHJ5*02 or IGHJ6*02 with up to 1, 2, 3, 4 or 5 amino acid alterations.

In one embodiment, the antibody comprises an antibody VH domain which
i) is derived from recombination of a human heavy chain V gene segment IGHV4-4*02, IGHV3-48*02, IGHV3-53*01, IGHV3-33*01 or IGHV1-24*d01, a human heavy chain D gene segment and a human heavy chain J gene segment, or
ii) comprises framework regions FR1, FR2, FR3 and FR4, wherein FR1, FR2 and FR3 each align with human germline V segment IGHV4-4*02, IGHV3-48*02, IGHV3-53*01, IGHV3-33*01 or IGHV1-24*d01 with up to 1, 2, 3, 4 or 5 amino acid alterations.

In one embodiment, the J gene segment is IGHJ4*02, IGHJ5*02 or IGHJ6*02, or the VH domain framework region FR4 aligns with human germline J gene segment IGHJ4*02, IGHJ5*02 or IGHJ6*02 with 1, 2, 3, 4 or 5 amino acid alterations.

In one embodiment, the antibody comprises an antibody VL domain which
i) is derived from recombination of a human light chain V gene segment and a human light chain J gene segment, wherein the V gene segment is IGLV1-40*01, IGLV3-21*d01, IGKV1D-16*01, IGKV1-9*d01, IGKV1D-17*01 or IGLV1-47*01; and/or the J gene segment is IGLJ3*02, IGLJ2*01, IGKJ4*01 or IGKJ1*01; or
ii) comprises framework regions FR1, FR2, FR3 and FR4, wherein FR1 aligns with human germline V gene segment IGLV1-40*01, IGLV3-21*d01, IGKV1D-16*01, IGKV1-9*d01, IGKV1D-17*01 or IGLV1-47*01 with up to 1, 2, 3, 4, or 5 amino acid alterations, FR2 aligns with human germline V gene segment IGLV1-40*01, IGLV3-21*d01, IGKV1D-16*01, IGKV1-9*d01, IGKV1D-17*01 or IGLV1-47*01 with up to 1, 2, 3, 4, or 5 amino acid alterations FR3 aligns with human germline V gene segment IGLV1-40*01, IGLV3-21*d01, IGKV1D-16*01, IGKV1-9*d01, IGKV1D-17*01 or IGLV1-47*01 with up to 1, 2, 3, 4 or 5 amino acid alterations, and/or FR4 aligns with human germline J gene segment IGLJ3*02, IGLJ2*01, IGKJ4*01 or IGKJ1*01 with up to 1, 2, 3, 4 or 5 amino acid alterations.

In one embodiment, the antibody comprises an antibody VL domain derived from recombination of a human light chain V gene segment and a human light chain J gene segment, wherein: the V gene segment is IGLV1-40*01, IGLV3-21*d01, IGKV1D-16*01, IGKV1-9*d01, IGKV1D-17*01 or IGLV1-47*01, and optionally the J gene segment is IGLJ3*02, IGLJ2*01, IGKJ4*01 or IGKJ1*01.

Example combinations of v and j gene segments for heavy and light chain variable domains are shown in Table 3, and these represent preferred combinations. The heavy and light chain variable domains may optionally be derived from the v and j gene segments identified in Table 3 for any one individual YANG antibody.

### Group E—NTD Binders

According to a fifth aspect of the invention, antibodies are provided herein which specifically binds to the N-terminal domain (NTD) of the S1 subunit of the SARS-CoV-2 spike protein. For example, antibodies YANG-1301, YANG-1302, YANG-1303, YANG-1304, YANG-1305; YANG-2301, YANG-2302, YANG-2303, YANG-2304, YANG-2305, and YANG-2306 as exemplified herein have been found to specifically bind to the N-terminal domain (NTD) of the S1 subunit of the SARS-CoV-2 spike protein.

In a first aspect, the present invention provides an antibody that specifically binds to the N-terminal domain (NTD) of the S1 subunit of the SARS-CoV-2 spike protein.

In one embodiment, the antibody specifically binds the N-terminal domain (NTD) of the S1 subunit of the SARS-CoV-2 spike protein with a KD of 0.1 nM or lower (e.g. as measured by surface plasmon resonance (SPR)).

In one embodiment, the antibody is a neutralising antibody.

In one embodiment, the antibody neutralises SARS-CoV-2 with an IC50 of 5.5 nM or lower (e.g. as measured in a pseudovirus neutralisation assay).

In one embodiment, the antibody specifically binds the N-terminal domain (NTD) of the S1 subunit of the SARS-CoV-2 spike protein with a KD of 0.1 nM or lower (e.g. as measured by surface plasmon resonance (SPR)) and the antibody neutralises SARS-CoV2 with an IC50 of 5.5 nM or lower (e.g. as measured in a pseudovirus neutralisation assay).

In one embodiment, the antibody comprises a variable heavy (VH) domain sequence comprising complementarity determining regions (CDRs) HCDR1, HCDR2 and HCDR3, and a variable light (VL) domain sequence comprising complementarity determining regions LCDR1, LCDR2 and LCDR3, wherein the HCDR3 is the HCDR3 of antibody YANG-1301, YANG-1302, YANG-1303, YANG-1304, YANG-1305; YANG-2301, YANG-2302, YANG-2303, YANG-2304, YANG-2305, or YANG-2306.

In one embodiment, HCDR3 is the HCDR3 of antibody YANG-1301.

In one embodiment, HCDR3 is the HCDR3 of antibody YANG-1302.

In one embodiment, HCDR3 is the HCDR3 of antibody YANG-1303.

In one embodiment, HCDR3 is the HCDR3 of antibody YANG-1304.

In one embodiment, HCDR3 is the HCDR3 of antibody YANG-1305.

In one embodiment, HCDR3 is the HCDR3 of antibody YANG-2301.

In one embodiment, HCDR3 is the HCDR3 of antibody YANG-2302.

In one embodiment, HCDR3 is the HCDR3 of antibody YANG-2303.

In one embodiment, HCDR3 is the HCDR3 of antibody YANG-2304.

In one embodiment, HCDR3 is the HCDR3 of antibody YANG-2305.

In one embodiment, HCDR3 is the HCDR3 of antibody YANG-2306.

In one embodiment, the antibody specifically binds the N-terminal domain (NTD) of the S1 subunit of the SARS-CoV-2 spike protein with a KD of 0.1 nM or lower (e.g. as measured by surface plasmon resonance (SPR)) and HCDR3 is the HCDR3 of antibody YANG-1301, YANG-1302, YANG-1303, YANG-1304, YANG-1305; YANG-2301, YANG-2302, YANG-2303, YANG-2304, YANG-2305, or YANG-2306.

In one embodiment, the antibody neutralises SARS-CoV-2 with an IC50 of 5.5 nM or lower (e.g. as measured in a pseudovirus neutralisation assay) and HCDR3 is the HCDR3 of antibody YANG-1301, YANG-1302, YANG-1303, YANG-1304, YANG-1305; YANG-2301, YANG-2302, YANG-2303, YANG-2304, YANG-2305, or YANG-2306.

In one embodiment, the antibody specifically binds the N-terminal domain (NTD) of the S1 subunit of the SARS-CoV-2 spike protein with a KD of 0.1 nM or lower (e.g. as measured by surface plasmon resonance (SPR)) and neutralises SARS-CoV-2 with an IC50 of 5.5 nM or lower (e.g. as measured in a pseudovirus neutralisation assay), and HCDR3 is the HCDR3 of antibody YANG-1301, YANG-1302, YANG-1303, YANG-1304, YANG-1305; YANG-2301, YANG-2302, YANG-2303, YANG-2304, YANG-2305, or YANG-2306.

In one embodiment, the present invention provides an anti-SARS-CoV-2 antibody, wherein the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3, wherein the CDRs are those of antibody YANG-1301, YANG-1302, YANG-1303, YANG-1304, YANG-1305; YANG-2301, YANG-2302, YANG-2303, YANG-2304, YANG-2305, or YANG-2306.

In one embodiment, the antibody has the CDRs of antibody YANG-1301.

In one embodiment, the antibody has the CDRs of antibody YANG-1302.

In one embodiment, the antibody has the CDRs of antibody YANG-1303.

In one embodiment, the antibody has the CDRs of antibody YANG-1304.

In one embodiment, the antibody has the CDRs of antibody YANG-1305.

In one embodiment, the antibody has the CDRs of antibody YANG-2301.

In one embodiment, the antibody has the CDRs of antibody YANG-2302.

In one embodiment, the antibody has the CDRs of antibody YANG-2303.

In one embodiment, the antibody has the CDRs of antibody YANG-2304.

In one embodiment, the antibody has the CDRs of antibody YANG-2305.

In one embodiment, the antibody has the CDRs of antibody YANG-2306.

In one embodiment, the present invention provides an anti-SARS-CoV-2 antibody, wherein the antibody specifically binds the N-terminal domain (NTD) of the S1 subunit ofthe SARS-CoV-2 spike protein with a KD of 0.1 nM or lower (e.g. as measured by surface plasmon resonance (SPR)), and wherein the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3, wherein the CDRs are those of antibody YANG-1301, YANG-1302, YANG-1303, YANG-1304, YANG-1305; YANG-2301, YANG-2302, YANG-2303, YANG-2304, YANG-2305, or YANG-2306.

In one embodiment, the present invention provides an anti-SARS-CoV-2 antibody, wherein the antibody neutralises SARS-CoV-2 with an IC50 of 5.5 nM or lower (e.g. as measured in a pseudovirus neutralisation assay), and wherein the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3, wherein the CDRs are those of antibody YANG-1301, YANG-1302, YANG-1303, YANG-1304, YANG-1305; YANG-2301, YANG-2302, YANG-2303, YANG-2304, YANG-2305, or YANG-2306.

In one embodiment, the present invention provides an anti-SARS-CoV-2 antibody, wherein the antibody specifically binds the N-terminal domain (NTD) of the S1 subunit ofthe SARS-CoV-2 spike protein with a KD of 0.1 nM or lower (e.g. as measured by surface plasmon resonance (SPR)) and neutralises SARS-CoV-2 with an IC50 of 5.5 nM or lower (e.g. as measured in a pseudovirus neutralisation assay), and wherein the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3, wherein the CDRs are those of antibody YANG-1301, YANG-1302, YANG-1303, YANG-1304, YANG-1305; YANG-2301, YANG-2302, YANG-2303, YANG-2304, YANG-2305, or YANG-2306.

In one embodiment, the antibody comprises a variable heavy (VH) domain sequence and a variable light (VL) domain sequence and wherein the variable heavy (VH) domain and variable light (VL) domain sequences respectively comprise the variable heavy (VH) domain and variable light (VL) domain sequences of antibody YANG-1301, YANG-1302, YANG-1303, YANG-1304, YANG-1305; YANG-2301, YANG-2302, YANG-2303, YANG-2304, YANG-2305, or YANG-2306, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

In one embodiment, the antibody comprises a variable heavy (VH) domain sequence and a variable light (VL) domain sequence and wherein the variable heavy (VH) domain and variable light (VL) domain sequences respectively comprise the variable heavy (VH) domain and variable light (VL) domain sequences of antibody YANG-1301, YANG-1302, YANG-1303, YANG-1304, YANG-1305; YANG-2301, YANG-2302, YANG-2303, YANG-2304, YANG-2305, or YANG-2306, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

In one embodiment, the antibody comprises a variable heavy (VH) domain sequence of antibody YANG-1301, optionally with 1, 2, 3, 4 or 5 amino acid alterations outside the complementarity determining regions (CDRs), and a variable light (VL) domain sequence of antibody YANG-1301, optionally with 1, 2, 3, 4 or 5 amino acid alterations outside the complementarity determining regions (CDRs).

In one embodiment, the antibody comprises a variable heavy (VH) domain sequence of antibody YANG-1302, optionally with 1, 2, 3, 4 or 5 amino acid alterations outside the complementarity determining regions (CDRs), and a variable light (VL) domain sequence of antibody YANG-1302, optionally with 1, 2, 3, 4 or 5 amino acid alterations outside the complementarity determining regions (CDRs).

In one embodiment, the antibody comprises a variable heavy (VH) domain sequence of antibody YANG-1303, optionally with 1, 2, 3, 4 or 5 amino acid alterations outside the complementarity determining regions (CDRs), and a variable light (VL) domain sequence of antibody YANG-1303, optionally with 1, 2, 3, 4 or 5 amino acid alterations outside the complementarity determining regions (CDRs).

In one embodiment, the antibody comprises a variable heavy (VH) domain sequence of antibody YANG-1304, optionally with 1, 2, 3, 4 or 5 amino acid alterations outside the complementarity determining regions (CDRs), and a variable light (VL) domain sequence of antibody YANG-1304, optionally with 1, 2, 3, 4 or 5 amino acid alterations outside the complementarity determining regions (CDRs).

In one embodiment, the antibody comprises a variable heavy (VH) domain sequence of antibody YANG-1305, optionally with 1, 2, 3, 4 or 5 amino acid alterations outside the complementarity determining regions (CDRs), and a variable light (VL) domain sequence of antibody YANG-1305, optionally with 1, 2, 3, 4 or 5 amino acid alterations outside the complementarity determining regions (CDRs).

In one embodiment, the antibody comprises a variable heavy (VH) domain sequence of antibody YANG-2301, optionally with 1, 2, 3, 4 or 5 amino acid alterations outside the complementarity determining regions (CDRs), and a variable light (VL) domain sequence of antibody YANG-2301, optionally with 1, 2, 3, 4 or 5 amino acid alterations outside the complementarity determining regions (CDRs).

In one embodiment, the antibody comprises a variable heavy (VH) domain sequence of antibody YANG-2302, optionally with 1, 2, 3, 4 or 5 amino acid alterations outside the complementarity determining regions (CDRs), and a variable light (VL) domain sequence of antibody YANG-2302, optionally with 1, 2, 3, 4 or 5 amino acid alterations outside the complementarity determining regions (CDRs).

In one embodiment, the antibody comprises a variable heavy (VH) domain sequence of antibody YANG-2303, optionally with 1, 2, 3, 4 or 5 amino acid alterations outside the complementarity determining regions (CDRs), and a variable light (VL) domain sequence of antibody YANG-2303, optionally with 1, 2, 3, 4 or 5 amino acid alterations outside the complementarity determining regions (CDRs).

In one embodiment, the antibody comprises a variable heavy (VH) domain sequence of antibody YANG-2304, optionally with 1, 2, 3, 4 or 5 amino acid alterations outside the complementarity determining regions (CDRs), and a variable light (VL) domain sequence of antibody YANG-2304, optionally with 1, 2, 3, 4 or 5 amino acid alterations outside the complementarity determining regions (CDRs).

In one embodiment, the antibody comprises a variable heavy (VH) domain sequence of antibody YANG-2305, optionally with 1, 2, 3, 4 or 5 amino acid alterations outside the complementarity determining regions (CDRs), and a variable light (VL) domain sequence of antibody YANG-2305, optionally with 1, 2, 3, 4 or 5 amino acid alterations outside the complementarity determining regions (CDRs).

In one embodiment, the antibody comprises a variable heavy (VH) domain sequence of antibody YANG-2306, optionally with 1, 2, 3, 4 or 5 amino acid alterations outside the complementarity determining regions (CDRs), and a variable light (VL) domain sequence of antibody YANG-2306, optionally with 1, 2, 3, 4 or 5 amino acid alterations outside the complementarity determining regions (CDRs).

In one embodiment, the antibody specifically binds the N-terminal domain (NTD) of the S1 subunit of the SARS-CoV-2 spike protein with a KD of 0.1 nM or lower (e.g. as measured by surface plasmon resonance (SPR)), and the variable heavy (VH) domain and variable light (VL) domain sequences respectively comprise the variable heavy (VH) domain and variable light (VL) domain sequences of antibody YANG-1301, YANG-1302, YANG-1303, YANG-1304, YANG-1305; YANG-2301, YANG-2302, YANG-2303, YANG-2304, YANG-2305, or YANG-2306, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

In one embodiment, the antibody neutralises SARS-CoV-2 with an IC50 of 5.5 nM or lower (e.g. as measured in a pseudovirus neutralisation assay), and and the variable heavy (VH) domain and variable light (VL) domain sequences respectively comprise the variable heavy (VH) domain and variable light (VL) domain sequences of antibody YANG-1301, YANG-1302, YANG-1303, YANG-1304, YANG-1305; YANG-2301, YANG-2302, YANG-2303, YANG-2304, YANG-2305, or YANG-2306, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

In one embodiment, the antibody specifically binds the N-terminal domain (NTD) of the S1 subunit of the SARS-CoV-2 spike protein with a KD of 0.1 nM or lower (e.g. as measured by surface plasmon resonance (SPR)) and neutralises SARS-CoV-2 with an IC50 of 5.5 nM or lower (e.g. as measured in a pseudovirus neutralisation assay), and and the variable heavy (VH) domain and variable light (VL) domain sequences respectively comprise the variable heavy (VH) domain and variable light (VL) domain sequences of antibody YANG-1301, YANG-1302, YANG-1303, YANG-1304, YANG-1305; YANG-2301, YANG-2302, YANG-2303, YANG-2304, YANG-2305, or YANG-2306, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

In one embodiment, the antibody comprises a variable heavy (VH) domain sequence and a variable light (VL) domain sequence and wherein the variable heavy (VH) domain and variable light (VL) domain sequences respectively comprise a sequence having at least 90% identity to the variable heavy (VH) and variable light (VL) domain sequences of antibody YANG-1301, YANG-1302, YANG-1303, YANG-1304, YANG-1305; YANG-2301, YANG-2302, YANG-2303, YANG-2304, YANG-2305, or YANG-2306 provided that the antibody has the CDRs of antibody YANG-1301, YANG-1302, YANG-1303, YANG-1304, YANG-1305; YANG-2301, YANG-2302, YANG-2303, YANG-2304, YANG-2305, or YANG-2306, respectively.

In one embodiment, the variable heavy (VH) domain sequence comprises a sequence having at least 90% identity to the VH domain sequence of antibody YANG-1301 and the variable light (VL) domain sequence comprises a sequence having at least 90% identity to the VL domain sequence of antibody YANG-1301, provided that the antibody has the CDRs of antibody YANG-1301.

In one embodiment, the variable heavy (VH) domain sequence comprises a sequence having at least 90% identity to the VH domain sequence of antibody YANG-1302 and the variable light (VL) domain sequence comprises a sequence having at least 90% identity to the VL domain sequence of antibody YANG-1302, provided that the antibody has the CDRs of antibody YANG-1302.

In one embodiment, the variable heavy (VH) domain sequence comprises a sequence having at least 90% identity to the VH domain sequence of antibody YANG-1303 and the variable light (VL) domain sequence comprises a sequence having at least 90% identity to the VL domain sequence of antibody YANG-1303, provided that the antibody has the CDRs of antibody YANG-1303.

In one embodiment, the variable heavy (VH) domain sequence comprises a sequence having at least 90% identity to the VH domain sequence of antibody YANG-1304 and the variable light (VL) domain sequence comprises a sequence having at least 90% identity to the VL domain sequence of antibody YANG-1304, provided that the antibody has the CDRs of antibody YANG-1304.

In one embodiment, the variable heavy (VH) domain sequence comprises a sequence having at least 90% identity to the VH domain sequence of antibody YANG-1305 and the variable light (VL) domain sequence comprises a sequence having at least 90% identity to the VL domain sequence of antibody YANG-1305, provided that the antibody has the CDRs of antibody YANG-1305.

In one embodiment, the variable heavy (VH) domain sequence comprises a sequence having at least 90% identity to the VH domain sequence of antibody YANG-2301 and the variable light (VL) domain sequence comprises a sequence having at least 90% identity to the VL domain sequence of antibody YANG-2301, provided that the antibody has the CDRs of antibody YANG-2301.

In one embodiment, the variable heavy (VH) domain sequence comprises a sequence having at least 90% identity to the VH domain sequence of antibody YANG-2302 and the variable light (VL) domain sequence comprises a sequence having at least 90% identity to the VL domain sequence of antibody YANG-2302, provided that the antibody has the CDRs of antibody YANG-2302.

In one embodiment, the variable heavy (VH) domain sequence comprises a sequence having at least 90% identity to the VH domain sequence of antibody YANG-2303 and the variable light (VL) domain sequence comprises a sequence having at least 90% identity to the VL domain sequence of antibody YANG-2303, provided that the antibody has the CDRs of antibody YANG-2303.

In one embodiment, the variable heavy (VH) domain sequence comprises a sequence having at least 90% identity to the VH domain sequence of antibody YANG-2304 and the variable light (VL) domain sequence comprises a sequence having at least 90% identity to the VL domain sequence of antibody YANG-2304, provided that the antibody has the CDRs of antibody YANG-2304.

In one embodiment, the variable heavy (VH) domain sequence comprises a sequence having at least 90% identity to the VH domain sequence of antibody YANG-2305 and the variable light (VL) domain sequence comprises a sequence having at least 90% identity to the VL domain sequence of antibody YANG-2305, provided that the antibody has the CDRs of antibody YANG-2305.

In one embodiment, the variable heavy (VH) domain sequence comprises a sequence having at least 90% identity to the VH domain sequence of antibody YANG-2306 and the variable light (VL) domain sequence comprises a sequence having at least 90% identity to the VL domain sequence of antibody YANG-2306, provided that the antibody has the CDRs of antibody YANG-2306.

In one embodiment, the antibody specifically binds the N-terminal domain (NTD) of the S 1 subunit of the SARS-CoV-2 spike protein with a KD of 0.1 nM or lower (e.g. as measured by surface plasmon resonance (SPR)), and and the antibody comprises a variable heavy (VH) domain sequence and a variable light (VL) domain sequence and wherein the variable heavy (VH) domain and variable light (VL) domain sequences respectively comprise a sequence having at least 90% identity to the variable heavy (VH) and variable light (VL) domain sequences of antibody YANG-1301, YANG-1302, YANG-1303, YANG-1304, YANG-1305; YANG-2301, YANG-2302, YANG-2303, YANG-2304, YANG-2305, or YANG-2306, provided that the antibody has the CDRs of antibody YANG-1301, YANG-1302, YANG-1303, YANG-1304, YANG-1305; YANG-2301, YANG-2302, YANG-2303, YANG-2304, YANG-2305, or YANG-2306, respectively.

In one embodiment, the antibody neutralises SARS-CoV-2 with an IC50 of 5.5 nM or lower (e.g. as measured in a pseudovirus neutralisation assay), and and the antibody comprises a variable heavy (VH) domain sequence and a variable light (VL) domain sequence and wherein the variable heavy (VH) domain and variable light (VL) domain sequences respectively comprise a sequence having at least 90% identity to the variable heavy (VH) and variable light (VL) domain sequences of antibody YANG-1301, YANG-1302, YANG-1303, YANG-1304, YANG-1305; YANG-2301, YANG-2302, YANG-2303, YANG-2304, YANG-2305, or YANG-2306, provided that the antibody has the CDRs of antibody YANG-1301, YANG-1302, YANG-1303, YANG-1304, YANG-1305; YANG-2301, YANG-2302, YANG-2303, YANG-2304, YANG-2305, or YANG-2306, respectively.

In one embodiment, the antibody specifically binds the N-terminal domain (NTD) of the S1 subunit of the SARS-CoV-2 spike protein with a KD of 0.1 nM or lower (e.g. as measured by surface plasmon resonance (SPR)) and neutralises SARS-CoV-2 with an IC50 of 5.5 nM or lower (e.g. as measured in a pseudovirus neutralisation assay), and and the antibody comprises a variable heavy (VH) domain sequence and a variable light (VL) domain sequence and wherein the variable heavy (VH) domain and variable light (VL) domain sequences respectively comprise a sequence having at least 90% identity to the variable heavy (VH) and variable light (VL) domain sequences of antibody YANG-1301, YANG-1302, YANG-1303, YANG-1304, YANG-1305; YANG-2301, YANG-2302, YANG-2303, YANG-2304, YANG-2305, or YANG-2306, provided that the antibody has the CDRs of antibody YANG-1301, YANG-1302, YANG-1303, YANG-1304, YANG-1305; YANG-2301, YANG-2302, YANG-2303, YANG-2304, YANG-2305, or YANG-2306, respectively.

In one embodiment, the present invention provides an antibody that specifically binds to the N-terminal domain (NTD) of the S1 subunit of the SARS-CoV-2 spike protein, wherein the antibody comprises a variable heavy (VH) domain sequence and a variable light (VL) domain sequence and wherein the variable heavy (VH) domain and variable light (VL) domain sequences respectively comprise the variable heavy (VH) and variable light (VL) domain sequences of antibody YANG-1301, YANG-1302, YANG-1303, YANG-1304, YANG-1305; YANG-2301, YANG-2302, YANG-2303, YANG-2304, YANG-2305, or YANG-2306.

In one embodiment, the antibody specifically binds to the N-terminal domain (NTD) of the S1 subunit of the SARS-CoV-2 spike protein, wherein the antibody comprises the VH and VL domain sequences of antibody YANG-1301.

In one embodiment, the antibody specifically binds to the N-terminal domain (NTD) of the S1 subunit of the SARS-CoV-2 spike protein, wherein the antibody comprises the VH and VL domain sequences of antibody YANG-1302.

In one embodiment, the antibody specifically binds to the N-terminal domain (NTD) of the S1 subunit of the SARS-CoV-2 spike protein, wherein the antibody comprises the VH and VL domain sequences of antibody YANG-1303.

In one embodiment, the antibody specifically binds to the N-terminal domain (NTD) of the S1 subunit of the SARS-CoV-2 spike protein, wherein the antibody comprises the VH and VL domain sequences of antibody YANG-1304.

In one embodiment, the antibody specifically binds to the N-terminal domain (NTD) of the S1 subunit of the SARS-CoV-2 spike protein, wherein the antibody comprises the VH and VL domain sequences of antibody YANG-1305.

In one embodiment, the antibody specifically binds to the N-terminal domain (NTD) of the S1 subunit of the SARS-CoV-2 spike protein, wherein the antibody comprises the VH and VL domain sequences of antibody YANG-2301.

In one embodiment, the antibody specifically binds to the N-terminal domain (NTD) of the S1 subunit of the SARS-CoV-2 spike protein, wherein the antibody comprises the VH and VL domain sequences of antibody YANG-2302.

In one embodiment, the antibody specifically binds to the N-terminal domain (NTD) of the S1 subunit of the SARS-CoV-2 spike protein, wherein the antibody comprises the VH and VL domain sequences of antibody YANG-2303.

In one embodiment, the antibody specifically binds to the N-terminal domain (NTD) of the S1 subunit of the SARS-CoV-2 spike protein, wherein the antibody comprises the VH and VL domain sequences of antibody YANG-2304.

In one embodiment, the antibody specifically binds to the N-terminal domain (NTD) of the S1 subunit of the SARS-CoV-2 spike protein, wherein the antibody comprises the VH and VL domain sequences of antibody YANG-2305.

In one embodiment, the antibody specifically binds to the N-terminal domain (NTD) of the S1 subunit of the SARS-CoV-2 spike protein, wherein the antibody comprises the VH and VL domain sequences of antibody YANG-2306.

In one embodiment, the antibody comprises a variable heavy (VH) domain sequence and a variable light (VL) domain sequence and wherein the variable heavy (VH) domain and variable light (VL) domain sequences respectively comprise the variable heavy (VH) and variable light (VL) domain sequences of antibody YANG-1301, YANG-1302, YANG-1303, YANG-1304, YANG-1305; YANG-2301, YANG-2302, YANG-2303, YANG-2304, YANG-2305, or YANG-2306.

In one embodiment, the antibody comprises the VH and VL domain sequences of antibody YANG-1301.

In one embodiment, the antibody comprises the VH and VL domain sequences of antibody YANG-1302.

In one embodiment, the antibody comprises the VH and VL domain sequences of antibody YANG-1303.

In one embodiment, the antibody comprises the VH and VL domain sequences of antibody YANG-1304.

In one embodiment, the antibody comprises the VH and VL domain sequences of antibody YANG-1305.

In one embodiment, the antibody comprises the VH and VL domain sequences of antibody YANG-2301.

In one embodiment, the antibody comprises the VH and VL domain sequences of antibody YANG-2302.

In one embodiment, the antibody comprises the VH and VL domain sequences of antibody YANG-2303.

In one embodiment, the antibody comprises the VH and VL domain sequences of antibody YANG-2304.

In one embodiment, the antibody comprises the VH and VL domain sequences of antibody YANG-2305.

In one embodiment, the antibody comprises the VH and VL domain sequences of antibody YANG-2306.

Antibodies are provided which bind to the same epitope on the N-terminal domain (NTD) of the S1 subunit of the SARS-CoV-2 spike protein as an antibody described anywhere herein.

An antibody is provided which bind to the same epitope as antibody YANG-1301, YANG-1302, YANG-1303, YANG-1304, YANG-1305; YANG-2301, YANG-2302, YANG-2303, YANG-2304, YANG-2305, or YANG-2306, e.g. as defined by its VH and VL sequences.

In one embodiment, an antibody is provided which binds to the same epitope as antibody YANG-1301.

In one embodiment, an antibody is provided which binds to the same epitope as antibody YANG-1302.

In one embodiment, an antibody is provided which binds to the same epitope as antibody YANG-1303.

In one embodiment, an antibody is provided which binds to the same epitope as antibody YANG-1304.

In one embodiment, an antibody is provided which binds to the same epitope as antibody YANG-1305.

In one embodiment, an antibody is provided which binds to the same epitope as antibody YANG-2301.

In one embodiment, an antibody is provided which binds to the same epitope as antibody YANG-2302.

In one embodiment, an antibody is provided which binds to the same epitope as antibody YANG-2303.

In one embodiment, an antibody is provided which binds to the same epitope as antibody YANG-2304.

In one embodiment, an antibody is provided which binds to the same epitope as antibody YANG-2305.

In one embodiment, an antibody is provided which binds to the same epitope as antibody YANG-2306.

An antibody may contact the SARS-CoV-2 spike protein with a footprint that fully or partly overlaps with that of an antibody disclosed anywhere herein. As described elsewhere herein, competition between antibodies may be determined, for example using SPR, and antibodies are provided which compete for binding to the spike protein (compete for binding to their epitope) with an IgG antibody as described anywhere herein.

An antibody of the present invention may be one which competes for binding to SARS-CoV-2 spike protein with any anti-NTD antibody described herein, such as YANG-1301, YANG-1302, YANG-1303, YANG-1304, YANG-1305; YANG-2301, YANG-2302, YANG-2303, YANG-2304, YANG-2305, or YANG-2306, e.g. as defined by its VH and VL sequences.

An antibody of the present invention may be one which competes for binding to the SARS-CoV-2 spike protein with antibody YANG-1301.

An antibody of the present invention may be one which competes for binding to the SARS-CoV-2 spike protein with antibody YANG-1302.

An antibody of the present invention may be one which competes for binding to the SARS-CoV-2 spike protein with any antibody YANG-1303.

An antibody of the present invention may be one which competes for binding to the SARS-CoV-2 spike protein with antibody YANG-1304.

An antibody of the present invention may be one which competes for binding to the SARS-CoV-2 spike protein with antibody YANG-1305.

An antibody of the present invention may be one which competes for binding to the SARS-CoV-2 spike protein with antibody YANG-2301.

An antibody of the present invention may be one which competes for binding to the SARS-CoV-2 spike protein with antibody YANG-2302.

An antibody of the present invention may be one which competes for binding to the SARS-CoV-2 spike protein with antibody YANG-2303.

An antibody of the present invention may be one which competes for binding to the SARS-CoV-2 spike protein with antibody YANG-2304.

An antibody of the present invention may be one which competes for binding to the SARS-CoV-2 spike protein with antibody YANG-2305.

An antibody of the present invention may be one which competes for binding to the SARS-CoV-2 spike protein with antibody YANG-2306.

In one embodiment, the antibody comprises VH and/or VL domain framework regions of human germline gene segment sequences.

In one embodiment, the antibody comprises an antibody VH domain which
i) is derived from recombination of a human heavy chain V gene segment, a human heavy chain D gene segment and a human heavy chain J gene segment, wherein the V gene segment is IGHV3-33*01, IGHV1-18*01, IGHV4-34*01, IGHV3-30*18 orIGHV1-24*d01; and/or the J gene segment is IGHJ5*02, IGHJ4*02 or IGHJ6*02; or
ii) comprises framework regions FR1, FR2, FR3 and FR4, wherein FR1 aligns with human germline V gene segment IGHV3-33*01, IGHV1-18*01, IGHV4-34*01, IGHV3-30*18 or IGHV1-24*d01 with up to 1, 2, 3, 4, or 5 amino acid alterations, FR2 aligns with human germline V gene segment IGHV3-33*01, IGHV1-18*01, IGHV4-34*01, IGHV3-30*18 or IGHV1-24*d01 with up to 1, 2, 3, 4, or 5 amino acid alterations, FR3 aligns with human germline V gene segment IGHV3-33*01, IGHV1-18*01, IGHV4-34*01, IGHV3-30*18 or IGHV1-24*d01 with up to 1, 2, 3, 4, or 5 amino acid alterations, and/or FR4 aligns with human germline J gene segment IGHJ5*02, IGHJ4*02 or IGHJ6*02 with up to 1, 2, 3, 4 or 5 amino acid alterations.

In one embodiment, the antibody comprises an antibody VH domain which
i) is derived from recombination of a human heavy chain V gene segment IGHV3-33*01, IGHV1-18*01, IGHV4-34*01, IGHV3-30*18 or IGHV1-24*d01, a human heavy chain D gene segment and a human heavy chain J gene segment, or
ii) comprises framework regions FR1, FR2, FR3 and FR4, wherein FR1, FR2 and FR3 each align with human germline V segment IGHV3-33*01, IGHV1-18*01, IGHV4-34*01, IGHV3-30*18 or IGHV1-24*d01 with up to 1, 2, 3, 4 or 5 amino acid alterations.

In one embodiment, the J gene segment is IGHJ5*02, IGHJ4*02 or IGHJ6*02, or the VH domain framework region FR4 aligns with human germline J gene segment IGHJ5*02, IGHJ4*02 or IGHJ6*02 with 1, 2, 3, 4 or 5 amino acid alterations.

In one embodiment, the antibody comprises an antibody VL domain which
i) is derived from recombination of a human light chain V gene segment and a human light chain J gene segment, wherein
   the V gene segment is IGLV3-1*01, IGLV3-10*01, IGKV1-27*01, IGKV3-15*01, IGKV3-20*01 or IGLV2-8*01; and/or
   the J gene segment is IGLJ2*01, IGLJ3*02, IGKJ3*01, IGKJ4*01 or IGKJ2*04; or
ii) comprises framework regions FR1, FR2, FR3 and FR4, wherein
   FR1 aligns with human germline V gene segment IGLV3-1*01, IGLV3-10*01, IGKV1-27*01, IGKV3-15*01, IGKV3-20*01 or IGLV2-8*01 with up to 1, 2, 3, 4, or 5 amino acid alterations,
   FR2 aligns with human germline V gene segment IGLV3-1*01, IGLV3-10*01, IGKV1-27*01, IGKV3-15*01, IGKV3-20*01 or IGLV2-8*01 with up to 1, 2, 3, 4, or 5 amino acid alterations,
   FR3 aligns with human germline V gene segment IGLV3-1*01, IGLV3-10*01, IGKV1-27*01, IGKV3-15*01, IGKV3-20*01 or IGLV2-8*01 with up to 1, 2, 3, 4 or 5 amino acid alterations, and/or
   FR4 aligns with human germline J gene segment IGLJ2*01, IGLJ3*02, IGKJ3*01, IGKJ4*01 or IGKJ2*04 with up to 1, 2, 3, 4 or 5 amino acid alterations.

In one embodiment, the antibody comprises an antibody VL domain derived from recombination of a human light chain V gene segment and a human light chain J gene segment, wherein: the V gene segment is IGLV3-1*01, IGLV3-10*01, IGKV1-27*01, IGKV3-15*01, IGKV3-20*01 or IGLV2-8*01, and optionally the J gene segment is IGLJ2*01, IGLJ3*02, IGKJ3*01, IGKJ4*01 or IGKJ2*04.

Example combinations of v and j gene segments for heavy and light chain variable domains are shown in Table 3, and these represent preferred combinations. The heavy and light chain variable domains may optionally be derived from the v and j gene segments identified in Table 3 for any one individual YANG antibody identified.

### Antibodies—General

In an embodiment, the antibody as defined anywhere herein shows ADCC activity.

In an embodiment, the antibody as defined anywhere herein does not cross-react with the existing endemic seasonal coronaviruses (NL63, 229E, OC43 and HKU1).

In an embodiment, the antibody as defined anywhere herein cross-reacts with SARS-CoV spike protein and/or MERS-CoV spike protein.

An antibody as defined anywhere herein may be a human IgG1 or human IgG4. In one embodiment, the antibody is a human IgG1. In one embodiment, the antibody is a human IgG1 comprising a constant region sequence of SEQ ID NO: 418. In one embodiment, the antibody is a human IgG4. In one embodiment, the antibody is a human IgG4 comprising a constant region sequence of SEQ ID NO: 436.

An antibody as defined anywhere herein may be a human IgA1 (e.g., comprising a constant region sequence SEQ ID NO: 484) or human IgA2 (e.g., comprising a constant region sequence SEQ ID NO: 485).

An antibody as defined anywhere herein may comprise kappa (κ) light chain constant regions, preferably constant domain sequence SEQ ID NO: 448.

Nucleic acid may comprise a sequence that encodes a VH domain and/or an VL domain of an antibody as defined anywhere herein. The nucleic acid may comprise a sequence that encodes a VH domain and/or a VL domain of antibody IMPI-029, IMPI-056, IMPI-005, IMPI-012, IMPI-052, IMPI-002, IMPI-041, IMPI-036, IMPI-055, IMPI-054, IMPI-042, IMPI-021, IMPI-004, IMPI-047, IMPI-017, IMPI-059, IMPI-030, IMPI-053, IMPI-025, IMPI-040, IMPI-007, IMPI-020, IMPI-032, IMPI-023, IMPI-039, IMPI-001, IMPI-019, IMPI-010, IMPI-008, IMPI-031, IMPI-057, IMPI-022, IMPI-035, IMPI-067, IMPI-072, IMPI-003, IMPI-013, IMPI-063, IMPI-061, IMPI-062, IMPI-064, IMPI-065, IMPI-066, IMPI-069, IMPI-070, IMPI-071; IMPI-026, IMPI-034, IMPI-016, IMPI-050, IMPI-049, IMPI-015, IMPI-009, IMPI-011, IMPI-044, IMPI-046, IMPI-051, IMPI-024, IMPI-058, IMPI-043, IMPI-045, IMPI-027, IMPI-018, IMPI-048, IMPI-033, IMPI-014, IMPI-028, IMPI-038 or IMPI-068.

Nucleic acid may comprise a sequence that encodes the VH domain of antibody IMPI-029, IMPI-056, IMPI-005, IMPI-012, IMPI-052, IMPI-002, IMPI-041, IMPI-036, IMPI-055, IMPI-054, IMPI-042, IMPI-021, IMPI-004, IMPI-047, IMPI-017, IMPI-059, IMPI-030, IMPI-053, IMPI-025, IMPI-040, IMPI-007, IMPI-020, IMPI-032, IMPI-023, IMPI-039, IMPI-001, IMPI-019, IMPI-010, IMPI-008, IMPI-031, IMPI-057, IMPI-022, IMPI-035, IMPI-067, IMPI-072, IMPI-003, IMPI-013, IMPI-063, IMPI-061, IMPI-062, IMPI-064, IMPI-065, IMPI-066, IMPI-069, IMPI-070, IMPI-071; IMPI-026, IMPI-034, IMPI-016, IMPI-050, IMPI-049, IMPI-015, IMPI-009, IMPI-011, IMPI-044, IMPI-046, IMPI-051, IMPI-024, IMPI-058, IMPI-043, IMPI-045, IMPI-027, IMPI-018, IMPI-048, IMPI-033, IMPI-014, IMPI-028, IMPI-038 or IMPI-068.

Nucleic acid may comprise a sequence that encodes the VL domain of antibody IMPI-029, IMPI-056, IMPI-005, IMPI-012, IMPI-052, IMPI-002, IMPI-041, IMPI-036, IMPI-055, IMPI-054, IMPI-042, IMPI-021, IMPI-004, IMPI-047, IMPI-017, IMPI-059, IMPI-030, IMPI-053, IMPI-025, IMPI-040, IMPI-007, IMPI-020, IMPI-032, IMPI-023, IMPI-039, IMPI-001, IMPI-019, IMPI-010, IMPI-008, IMPI-031, IMPI-057, IMPI-022, IMPI-035, IMPI-067, IMPI-072, IMPI-003, IMPI-013, IMPI-063, IMPI-061, IMPI-062, IMPI-064, IMPI-065, IMPI-066, IMPI-069, IMPI-070, IMPI-071; IMPI-026, IMPI-034, IMPI-016, IMPI-050, IMPI-049, IMPI-015, IMPI-009, IMPI-011, IMPI-044, IMPI-046, IMPI-051, IMPI-024, IMPI-058, IMPI-043, IMPI-045, IMPI-027, IMPI-018, IMPI-048, IMPI-033, IMPI-014, IMPI-028, IMPI-038 or IMPI-068.

A nucleic acid sequence provided by the invention may comprise a sequence that encodes a VH domain and/or an VL domain of an antibody as defined anywhere herein.

The nucleic acid may comprise a sequence that encodes a VH domain and/or a VL domain of antibody IMPI-037.

The nucleic acid may comprise a sequence that encodes a VH domain and/or a VL domain of antibody YANG-1101.

The nucleic acid may comprise a sequence that encodes a VH domain and/or a VL domain of antibody YANG-1103.

The nucleic acid may comprise a sequence that encodes a VH domain and/or a VL domain of antibody YANG-1105. nucleic acid may comprise a sequence that encodes a VH domain and/or a VL domain of antibody YANG-1106.

The nucleic acid may comprise a sequence that encodes a VH domain and/or a VL domain of antibody YANG-1107.

The nucleic acid may comprise a sequence that encodes a VH domain and/or a VL domain of antibody YANG-1108.

The nucleic acid may comprise a sequence that encodes a VH domain and/or a VL domain of antibody YANG-1109.

The nucleic acid may comprise a sequence that encodes a VH domain and/or a VL domain of antibody YANG-1110.

The nucleic acid may comprise a sequence that encodes a VH domain and/or a VL domain of antibody YANG-1112.

The nucleic acid may comprise a sequence that encodes a VH domain and/or a VL domain of antibody YANG-1113.

The nucleic acid may comprise a sequence that encodes a VH domain and/or a VL domain of antibody YANG-1114.

The nucleic acid may comprise a sequence that encodes a VH domain and/or a VL domain of antibody YANG-1115.

The nucleic acid may comprise a sequence that encodes a VH domain and/or a VL domain of antibody YANG-1116.

The nucleic acid may comprise a sequence that encodes a VH domain and/or a VL domain of antibody YANG-1117.

The nucleic acid may comprise a sequence that encodes a VH domain and/or a VL domain of antibody YANG-1118.

The nucleic acid may comprise a sequence that encodes a VH domain and/or a VL domain of antibody YANG-1119.

The nucleic acid may comprise a sequence that encodes a VH domain and/or a VL domain of antibody YANG-2101.

The nucleic acid may comprise a sequence that encodes a VH domain and/or a VL domain of antibody YANG-2102.

The nucleic acid may comprise a sequence that encodes a VH domain and/or a VL domain of antibody YANG-2103.

The nucleic acid may comprise a sequence that encodes a VH domain and/or a VL domain of antibody YANG-2104.

The nucleic acid may comprise a sequence that encodes a VH domain and/or a VL domain of antibody YANG-2105.

The nucleic acid may comprise a sequence that encodes a VH domain and/or a VL domain of antibody YANG-2106.

The nucleic acid may comprise a sequence that encodes a VH domain and/or a VL domain of antibody YANG-2107.

The nucleic acid may comprise a sequence that encodes a VH domain and/or a VL domain of antibody YANG-2108.

The nucleic acid may comprise a sequence that encodes a VH domain and/or a VL domain of antibody YANG-2109.

The nucleic acid may comprise a sequence that encodes a VH domain and/or a VL domain of antibody YANG-2110.

The nucleic acid may comprise a sequence that encodes a VH domain and/or a VL domain of antibody YANG-2111.

The nucleic acid may comprise a sequence that encodes a VH domain and/or a VL domain of antibody YANG-1201.

The nucleic acid may comprise a sequence that encodes a VH domain and/or a VL domain of antibody YANG-1202.

The nucleic acid may comprise a sequence that encodes a VH domain and/or a VL domain of antibody YANG-1203.

The nucleic acid may comprise a sequence that encodes a VH domain and/or a VL domain of antibody YANG-1204.

The nucleic acid may comprise a sequence that encodes a VH domain and/or a VL domain of antibody YANG-1205.

The nucleic acid may comprise a sequence that encodes a VH domain and/or a VL domain of antibody YANG-1206.

The nucleic acid may comprise a sequence that encodes a VH domain and/or a VL domain of antibody YANG-1207.

The nucleic acid may comprise a sequence that encodes a VH domain and/or a VL domain of antibody YANG-2201.

The nucleic acid may comprise a sequence that encodes a VH domain and/or a VL domain of antibody YANG-2202.

The nucleic acid may comprise a sequence that encodes a VH domain and/or a VL domain of antibody YANG-2203.

The nucleic acid may comprise a sequence that encodes a VH domain and/or a VL domain of antibody YANG-2204.

The nucleic acid may comprise a sequence that encodes a VH domain and/or a VL domain of antibody YANG-2205.

The nucleic acid may comprise a sequence that encodes a VH domain and/or a VL domain of antibody YANG-2206.

The nucleic acid may comprise a sequence that encodes a VH domain and/or a VL domain of antibody YANG-2207.

The nucleic acid may comprise a sequence that encodes a VH domain and/or a VL domain of antibody YANG-2208.

The nucleic acid may comprise a sequence that encodes a VH domain and/or a VL domain of antibody YANG-1102.

The nucleic acid may comprise a sequence that encodes a VH domain and/or a VL domain of antibody YANG-1111.

The nucleic acid may comprise a sequence that encodes a VH domain and/or a VL domain of antibody YANG-1401.

The nucleic acid may comprise a sequence that encodes a VH domain and/or a VL domain of antibody YANG-1402.

The nucleic acid may comprise a sequence that encodes a VH domain and/or a VL domain of antibody YANG-1403.

The nucleic acid may comprise a sequence that encodes a VH domain and/or a VL domain of antibody YANG-2112.

The nucleic acid may comprise a sequence that encodes a VH domain and/or a VL domain of antibody YANG-1301.

The nucleic acid may comprise a sequence that encodes a VH domain and/or a VL domain of antibody YANG-1302.

The nucleic acid may comprise a sequence that encodes a VH domain and/or a VL domain of antibody YANG-1303.

The nucleic acid may comprise a sequence that encodes a VH domain and/or a VL domain of antibody YANG-1304.

The nucleic acid may comprise a sequence that encodes a VH domain and/or a VL domain of antibody YANG-1305.

The nucleic acid may comprise a sequence that encodes a VH domain and/or a VL domain of antibody YANG-2301.

The nucleic acid may comprise a sequence that encodes a VH domain and/or a VL domain of antibody YANG-2302.

The nucleic acid may comprise a sequence that encodes a VH domain and/or a VL domain of antibody YANG-2303.

The nucleic acid may comprise a sequence that encodes a VH domain and/or a VL domain of antibody YANG-2304.

The nucleic acid may comprise a sequence that encodes a VH domain and/or a VL domain of antibody YANG-2305.

The nucleic acid may comprise a sequence that encodes a VH domain and/or a VL domain of antibody YANG-2306.

A vector may comprise the nucleic acid as defined anywhere herein; optionally wherein the vector is a CHO vector.

A host cell may comprise the nucleic acid as defined anywhere herein or the vector as defined anywhere herein.

A pharmaceutical composition may comprise an antibody as defined anywhere herein and a pharmaceutically acceptable excipient.

A pharmaceutical composition may comprise an isolated nucleic acid encoding an antibody as defined anywhere herein, or the isolated nucleic acid as defined anywhere herein, and a pharmaceutically acceptable excipient.

In one embodiment, the pharmaceutical composition is formulated for intravenous, intramuscular or subcutaneous administration.

In one embodiment, the pharmaceutical composition further comprises at least one further therapeutic agent.

In one embodiment, the further therapeutic agent is at least one, preferably one or two, further antibodies.

In one embodiment, the at least one further antibody is selected from:
an antibody that specifically binds to the receptor binding domain (RBD) of the S1 subunit of the SARS-CoV-2 spike protein and competes for binding to the SARS-CoV-2 spike protein with the human ACE2 receptor;
an antibody that specifically binds to the receptor binding domain (RBD) of the S1 subunit of the SARS-CoV-2 spike protein and does not compete for binding to the SARS-CoV-2 spike protein with the human ACE2 receptor;
an antibody that specifically binds to the N-terminal domain (NTD) of the S 1 subunit of the of SARS-CoV-2 spike protein;
an antibody that specifically binds to the S2 subunit of the of SARS-CoV-2 spike protein; and an antibody preferentially binds to the trimer form of the SARS-CoV-2 spike protein over the isolated RBD domain, S1 subunit and S2 subunit ofthe SARS-CoV-2 spike protein.

In one embodiment, the pharmaceutical composition comprises a first antibody that specifically binds to the receptor binding domain (RBD) of the S1 subunit of the SARS-CoV-2 spike protein and competes for binding to the SARS-CoV-2 spike protein with the human ACE2 receptor and a second antibody that specifically binds to the S2 subunit of the of SARS-CoV-2 spike protein. Such combinations have been found to advantageously inhibit syncytia formation. In this embodiment, the pharmaceutical composition may be capable of syncytia formation inhibition of 45% or greater or even 50% or greater (e.g. as measured in a syncytia formation inhibition assay as described herein). The first antibody may be an antibody according to the first aspect of the invention. The second antibody may be an antibody according to the third aspect of the invention. In this embodiment, the first antibody maybe IMPI-059 or an antibody having HCDR3, the 6 CDRs, or the VH and/or VL domain sequences of IMPI-059. In this embodiment, the second antibody may be YANG-2204, YANG-2206, and YANG-2207 or an antibody having HCDR3, the 6 CDRs, or the VH and/or VL domain sequences of YANG-2204, YANG-2206, and YANG-2207. In this embodiment, the first antibody may be an antibody having the 6 CDRs of IMPI-059 and the second antibody may an antibody having the 6 CDRs of YANG-2204. In this embodiment, the first antibody may be an antibody having the 6 CDRs of IMPI-059 and the second antibody may an antibody having the 6 CDRs of YANG-2206. In this embodiment, the first antibody may be an antibody having the 6 CDRs of IMPI-059 and the second antibody may an antibody having the 6 CDRs of YANG-2207. In this embodiment, the first antibody may be an antibody having the VH and VL domain sequences of IMPI-059 and the second antibody may an antibody having the VH and VL domain sequences of YANG-2204. In this embodiment, the first antibody may be an antibody having the VH and VL domain sequences of IMPI-059 and the second antibody may an antibody having the VH and VL domain sequences of YANG-2206. In this embodiment, the first antibody may be an antibody having the VH and VL domain sequences of IMPI-059 and the second antibody may an antibody having the VH and VL domain sequences of YANG-2207.

A kit may comprise the pharmaceutical composition as defined anywhere herein. In one embodiment, the kit further comprises at least one further therapeutic agent. In one embodiment, the further therapeutic agent is a further pharmaceutical composition comprising at least one, preferably one or two, further antibodies. In one embodiment, the at least one further antibody is selected from: an antibody that specifically binds to the receptor binding domain (RBD) of the S1 subunit of the SARS-CoV-2 spike protein and competes for binding to the SARS-CoV-2 spike protein with the human ACE2 receptor;
an antibody that specifically binds to the receptor binding domain (RBD) of the S1 subunit of the SARS-CoV-2 spike protein and does not compete for binding to the SARS-CoV-2 spike protein with the human ACE2 receptor;
an antibody that specifically binds to the N-terminal domain (NTD) of the S 1 subunit of the of SARS-CoV-2 spike protein;
an antibody that specifically binds to the S2 subunit of the of SARS-CoV-2 spike protein; and an antibody preferentially binds to the trimer form of the SARS-CoV-2 spike protein over the isolated RBD domain, S1 subunit and S2 subunit ofthe SARS-CoV-2 spike protein.

In one embodiment, the kit comprises a first antibody that specifically binds to the receptor binding domain (RBD) of the S1 subunit of the SARS-CoV-2 spike protein and competes for binding to the SARS-CoV-2 spike protein with the human ACE2 receptor and a second antibody that specifically binds to the S2 subunit of the of SARS-CoV-2 spike protein. Such combinations have been found to advantageously inhibit syncytia formation. In this embodiment, the kit may be capable of syncytia formation inhibition of 45% or greater or even 50% or greater (e.g. as measured in a syncytia formation inhibition assay as described herein). The first antibody may be an antibody according to the first aspect of the invention. The second antibody may be an antibody according to the third aspect of the invention. In this embodiment, the first antibody may be IMPI-059 or an antibody having HCDR3, the 6 CDRs, or the VH and/or VL domain sequences of IMPI-059. In this embodiment, the second antibody may be YANG-2204, YANG-2206, and YANG-2207 or an antibody having HCDR3, the 6 CDRs, or the VH and/or VL domain sequences of YANG-2204, YANG-2206, and YANG-2207. In this embodiment, the first antibody may be an antibody having the 6 CDRs of IMPI-059 and the second antibody may an antibody having the 6 CDRs of YANG-2204. In this embodiment, the first antibody may be an antibody having the 6 CDRs of IMPI-059 and the second antibody may an antibody having the 6 CDRs of YANG-2206. In this embodiment, the first antibody may be an antibody having the 6 CDRs of IMPI-059 and the second antibody may an antibody having the 6 CDRs of YANG-2207. In this embodiment, the first antibody may be an antibody having the VH and VL domain sequences of IMPI-059 and the second antibody may an antibody having the VH and VL domain sequences of YANG-2204. In this embodiment, the first antibody may be an antibody having the VH and VL domain sequences of IMPI-059 and the second antibody may an antibody having the VH and VL domain sequences of YANG-2206. In this embodiment, the first antibody may be an antibody having the VH and VL domain sequences of IMPI-059 and the second antibody may an antibody having the VH and VL domain sequences of YANG-2207.

In one embodiment, the kit further comprises a label or instructions for use to treat and/or prevent a SARS-CoV-2-related disease or condition, such as COVID-19, in a human; optionally wherein the label or instructions comprise a marketing authorisation number (e.g., an FDA or EMA authorisation number); optionally wherein the kit comprises an IV or injection device that comprises the antibody or fragment.

An antibody as defined anywhere herein or a composition as defined anywhere herein may be provided for use as a medicament.

The antibody as defined anywhere herein, or the composition as defined anywhere herein, may be provided for use in a method of treating a SARS-CoV-2-related disease or condition, said method comprising administering the antibody or composition to a patient.

The antibody as defined anywhere herein, or the composition as defined anywhere herein, may be provided for use in a method of preventing a SARS-CoV-2-related disease or condition, said method comprising administering the antibody or composition to a patient.

Also described is the use of an antibody as defined anywhere herein, or the composition as defined anywhere herein, in the manufacture of a medicament for use in a method of treating a SARS-CoV-2-related disease or condition.

Also described is the use of an antibody as defined anywhere herein, or the composition as defined anywhere herein, in the manufacture of a medicament for use in a method of preventing a SARS-CoV-2-related disease or condition.

A method of treating a SARS-CoV-2-related disease or condition in a human may comprise administering to said human a therapeutically effective amount of an antibody as defined anywhere herein, or the composition as defined anywhere herein.

A method of preventing a SARS-CoV-2-related disease or condition in a human may comprise administering to said human a therapeutically effective amount of an antibody as defined anywhere herein, or the composition as defined anywhere herein.

In one embodiment, the SARS-CoV-2-related disease or condition is a SARS-CoV-2-mediated disease or condition.

In one embodiment, the SARS-CoV-2-related disease or condition is a COVID-19-related disease or condition. In some examples, the COVID-19-related disease or condition is COVID-19. In some examples, the COVID-19-related disease or condition is a long manifestation of infection by SARS-CoV-2 such as ‘Long COVID’.

In one embodiment, the SARS-CoV-2-related disease or condition is COVID-19.

In one embodiment, the method further comprises administering at least one further therapeutic agent.

In one embodiment, the administration of the further therapeutic agent is simultaneous, separate or sequential.

In one embodiment, the further therapeutic agent is at least one, preferably one or two, further antibodies.

In one embodiment, the at least one further antibody is selected from:
an antibody that specifically binds to the receptor binding domain (RBD) of the S1 subunit of the SARS-CoV-2 spike protein and competes for binding to the SARS-CoV2 spike protein with the human ACE2 receptor;
an antibody that specifically binds to the receptor binding domain (RBD) of the S1 subunit of the SARS-CoV-2 spike protein and does not compete for binding to the SARS-CoV-2 spike protein with the human ACE2 receptor;
an antibody that specifically binds to the N-terminal domain (NTD) of the S 1 subunit of the of SARS-CoV-2 spike protein;
an antibody that specifically binds to the S2 subunit of the of SARS-CoV-2 spike protein; and an antibody preferentially binds to the trimer form of the SARS-CoV-2 spike protein over the isolated RBD domain, S1 subunit and S2 subunit ofthe SARS-CoV-2 spike protein.

In one embodiment, the method comprises administering a first antibody that specifically binds to the receptor binding domain (RBD) of the S1 subunit of the SARS-CoV-2 spike protein and competes for binding to the SARS-CoV-2 spike protein with the human ACE2 receptor and a second antibody that specifically binds to the S2 subunit of the of SARS-CoV-2 spike protein. Such combinations have been found to advantageously inhibit syncytia formation. In this embodiment, the method may be capable of syncytia formation inhibition of 45% or greater or even 50% or greater (e.g. as measured in a syncytia formation inhibition assay as described herein). The first antibody may be an antibody according to the first aspect of the invention. The second antibody may be an antibody according to the third aspect of the invention. In this embodiment, the first antibody may be IMPI-059 or an antibody having HCDR3, the 6 CDRs, or the VH and/or VL domain sequences of IMPI-059. In this embodiment, the second antibody may be YANG-2204, YANG-2206, and YANG-2207 or an antibody having HCDR3, the 6 CDRs, or the VH and/or VL domain sequences of YANG-2204, YANG-2206, and YANG-2207. In this embodiment, the first antibody may be an antibody having the 6 CDRs of IMPI-059 and the second antibody may an antibody having the 6 CDRs of YANG-2204. In this embodiment, the first antibody may be an antibody having the 6 CDRs of IMPI-059 and the second antibody may an antibody having the 6 CDRs of YANG-2206. In this embodiment, the first antibody may be an antibody having the 6 CDRs of IMPI-059 and the second antibody may an antibody having the 6 CDRs of YANG-2207. In this embodiment, the first antibody may be an antibody having the VH and VL domain sequences of IMPI-059 and the second antibody may an antibody having the VH and VL domain sequences of YANG-2204. In this embodiment, the first antibody may be an antibody having the VH and VL domain sequences of IMPI-059 and the second antibody may an antibody having the VH and VL domain sequences of YANG-2206. In this embodiment, the first antibody may be an antibody having the VH and VL domain sequences of IMPI-059 and the second antibody may an antibody having the VH and VL domain sequences of YANG-2207.

In one example, the first antibody and the second antibody may be administered simultaneously, separately, or sequentially.

Also described is the use of an antibody as defined anywhere herein, for determining the presence or absence of SARS-CoV-2 in a sample.

A method of determining the presence or absence of SARS-CoV-2 in a sample may comprise contacting the sample with an antibody as defined anywhere herein; and testing for binding between the antibody and SARS-CoV2 in the sample; wherein detection of binding indicates the presence of SARS-CoV-2 in the sample and wherein absence of binding indicates the absence of SARS-CoV-2 in the sample.

In one embodiment, the antibody comprises or is conjugated to a detectable label.

In one embodiment, the sample has been obtained from a human who has been or is suspected of having been infected with SARS-CoV-2 and/or who exhibits one or more symptoms of COVID-19. In one embodiment, the sample is a serum, plasma, or whole blood sample, an oral or nasal swab, urine, faeces, or cerebrospinal fluid (CFS), or wherein the sample is from any suspected SARS-CoV-2 infected organ or tissue.

A diagnostic kit for the use as defined anywhere herein, or the method as defined anywhere herein, may comprise an antibody as defined anywhere herein, and optionally one or more buffering solutions. In one embodiment, the diagnostic kit comprises a first reagent comprising the antibody as defined anywhere herein, and a second reagent comprising a detector molecule that binds to the first reagent. In one embodiment, the detector molecule is an antibody that comprises or is conjugated to a detectable label.

### BRIEF DESCRIPTION OF THE DRAWINGS

FIG. 1. depicts a single monomer of the SARS-CoV-2 virus trimeric spike protein (S).

FIGS. 2A-2B: Amino acid sequence of the SARS-CoV-2 spike protein, with residue numbering for the nascent polypeptide. The signal peptide (residues 1-13, bold italics) is cleaved off before secretion. During maturation the polypeptide is cleaved at the S1/S2 cleavage site (residues 681-686, boxed) into subunits S1 (residues 14-685) and S2 (residues 686-1273, bold). These subunits remain non-covalently attached. The S1 subunit is further sub-divided into two functional domains: the N-terminal domain (NTD) (residues 14-306, wavy underlined) and the Receptor Binding Domain (RBD) (residues 331-528, underlined). The S2 subunit contains a transmembrane-domain (residues 1212-1234, bold underlined); the ectodomain (ECD) of spike contains all membrane-distal residues of subunits S1 and S2 (residues 1-1211). Following secretion, the protein is further cleaved at the S2′ cleavage site (residues 815-816) upon cell adhesion, initiating a conformational change in the spike protein that leads to virus entry into the cell. To prevent the protein from spontaneously changing conformation in in vitro assays, a double-proline mutation (residues 986-987, dashed box) was introduced to stabilise the protein in its pre-fusion configuration. FIG. 2A depicts the wild-type spike protein amino acid sequence. FIG. 2B depicts the engineered spike protein sequence containing substitutions K986P and V987P.

FIGS. 3A-3Z: Alignments of exemplary anti-SARS-CoV-2 antibody light and heavy chain amino acid sequences showing exemplary anti-SARS-CoV-2 antibodies and their siblings based on sequence homology. In summary, clusters were generated as follows: B cells were isolated from the immunised mice and their antibody-encoding sequences were recovered. By comparing sequences of the heavy and light chain variable domains, we identified clusters of sequences which correspond to families of B cells within a lineage. These clusters share the same v and j gene segments in their heavy and light chain variable domains and the same HCDR3 length. Given their inferred in vivo evolutionary relationship, all siblings within a cluster may be expected to share similar qualitative properties such as epitope binding and mode of action, especially where siblings were obtained from B cells which were recovered by antigen specific sorting, even if assay data for those siblings are not provided herein.

FIG. 4: Diagram showing binding properties of exemplary antibodies described herein as determined by HTRF.

FIG. 5: Neutralisation of SARS CoV-2 pseudovirus by RBD binding ACE2 competing antibodies mAb A, mAb B, mAb C and SAD S35.

FIG. 6:

Neutralisation of SARS CoV2 pseudovirus by RBD ACE2 competing antibodies. Graph shows only antibodies that are equivalent to or more potent than the comparator antibodies mAb A, mAb B, mAb C and SAD S35 (dashed lines).

FIG. 7: Neutralisation of SARS CoV2 pseudovirus by NTD binding antibody 4A8 compared to a RBD ACE2 competing antibody SAD-S35 (dashed line).

FIG. 8: Neutralisation of SARS CoV2 pseudovirus by RBD binding but non-ACE2 competing antibodies.

An RBD ACE2 competing antibody, SAD-S35, is included for comparison.

FIG. 9: Neutralisation of SARS CoV2 pseudovirus by S2 binding antibodies. An RBD ACE2 competing antibody, SAD-S35, is included for comparison (dashed line).

FIG. 10: Example of a neutralising antibody in the HTRF ACE2:trimer neutralisation assay (IMPI-027) and an antibody (IMPI-059) that increases binding activity.

FIG. 11: HTRF ACE2:trimer neutralisation curves for antibodies IMPI-024 and IMPI-068 which increase binding activity (open symbols), and comparator antibodies (filled symbols) which decrease binding activity.

FIG. 12: Epitope cross-competition binning of clones by SPR. Grey boxes indicate the two antibodies compete with each other for binding to RBD, white boxes indicate no competition between the pair of antibodies. Black boxes indicate assays not undertaken as they are the same antibody.

FIG. 13: IC50 values obtained for antibodies in live virus plaque neutralisation assay. Note log scale for IC50. The two control mAbs COV2-2196 and COV2-2130 are presently (October 2020) clinical candidate therapeutic antibodies. The 4 IMPI mAbs, tested singly (i.e., in monoclonal compositions), are also shown. IMPI-059 was the most potent antibody in this assay. IMPI-013 is an S2 binding neutralising mAb. IMPI-017 had an IC50 of 326 pM and IMPI-004 had an IC50 of 86 pM, IMPI-059 had an IC50 of 26 pM and IMPI-013 had an TC50 of 3.4 nM.

FIG. 14: Results of the syncytia inhibition assay of Example 18.

FIG. 15: Showing cluster information for antibody YANG-1401 VH

FIG. 16: Showing cluster information for antibody YANG-1401 VL

FIG. 17: Showing cluster information for antibodies YANG-2107 and YANG-2108 VH

FIG. 18: Showing cluster information for antibodies YANG-2107 and YANG-2108 VL

FIG. 19: Showing cluster information for antibody YANG-2111 VH

FIG. 20: Showing cluster information for antibody YANG-2111 VL

FIG. 21: Showing cluster information for antibody YANG-2203 VH

FIG. 22: Showing cluster information for antibody YANG-2203 VL

FIG. 23A-C: Showing cluster information for antibodies YANG-2204, YANG-2205, YANG-2206, YANG-2207, and YANG-2208 VH

FIG. 24A-C: Showing cluster information for antibodies YANG-2204, YANG-2205, YANG-2206, YANG-2207, and YANG-2208 VL

FIG. 25: Showing cluster information for antibody YANG-1112 VH

FIG. 26: Showing cluster information for antibody YANG-1112 VL

### DETAILED DESCRIPTION

### Definitions

Unless otherwise defined herein, scientific and technical terms shall have the meanings that are commonly understood by those of ordinary skill in the art. Further, unless otherwise required by context, singular terms shall include pluralities and plural terms shall include the singular.

The singular terms “a,” “an,” and “the” include plural referents unless context clearly indicates otherwise. Similarly, the word “or” is intended to include “and” unless the context clearly indicates otherwise.

Although methods and materials similar or equivalent to those described herein can be used in the practice or testing of this disclosure, suitable methods and materials are described below. The abbreviation, “e.g.” is derived from the Latin exempli gratia and is used herein to indicate a non-limiting example. Thus, the abbreviation “e.g.” is synonymous with the term “for example.”

In the specification and claims, the term “about” is used to modify, for example, the quantity of an ingredient in a composition, concentration, volume, process temperature, process time, yield, flow rate, pressure, and like values, and ranges thereof, employed in describing the examples of the disclosure. The term “about” refers to variation in the numerical quantity that can occur, for example, through typical measuring and handling procedures used for making compounds, compositions, concentrates or use formulations; through inadvertent error in these procedures; through differences in the manufacture, source, or purity of starting materials or ingredients used to carry out the methods, and like proximate considerations. The term “about” also encompasses amounts that differ due to aging of a formulation with a particular initial concentration or mixture and amounts that differ due to mixing or processing a formulation with a particular initial concentration or mixture. Where modified by the term “about” the claims appended hereto include equivalents to these quantities.

As used herein, “administer” or “administration” refers to the act of injecting or otherwise physically delivering a substance as it exists outside the body (e.g., an anti-SARS-CoV-2 spike protein antibody provided herein, or its encoding nucleic acid e.g. in an expression vector) into a patient, such as by mucosal, intradermal, intravenous, intramuscular delivery, inhalation e.g. nebulisation and/or any other method of physical delivery described herein or known in the art. When a disease, or a symptom thereof, is being treated, administration of the substance typically occurs after the onset of the disease or symptoms thereof When a disease, or symptoms thereof, are being prevented, administration of the substance typically occurs before the onset of the disease or symptoms thereof.

The term “antibody”, “immunoglobulin” or “Ig” may be used interchangeably herein and means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, antibody fragments (such as Fab, Fab′, F(ab′)2, and Fv fragments), single chain Fv (scFv) mutants, multispecific antibodies such as bispecific antibodies (including dual binding antibodies), chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. The term “antibody” can also refer to a Y-shaped glycoprotein with a molecular weight of approximately 150 kDa that is made up of four polypeptide chains: two light (L) chains and two heavy (H) chains. There are five types of mammalian Ig heavy chain isotypes denoted by the Greek letters alpha (α), delta (δ), epsilon (ε), gamma (γ), and mu (μ). The type of heavy chain defines the class of antibody, i.e., IgA, IgD, IgE, IgG, and IgM, respectively. The γ and α classes are further divided into subclasses on the basis of differences in the constant domain sequence and function, e.g., IgG1, hIgG2, mIgG2A, mIgG2B, IgG3, IgG4, IgA1 and IgA2. In mammals, there are two types of immunoglobulin light chains, and K. The “variable region” or “variable domain” of an antibody refers to the amino-terminal domains of the heavy or light chain of the antibody. The variable domains of the heavy chain and light chain may be referred to as “VH” and “VL”, respectively. These domains are generally the most variable parts of the antibody (relative to other antibodies of the same class) and contain the antigen binding sites. An example of antibodies are heavy chain-only (i.e., H2) antibodies that comprise a dimer of a heavy chain (5′-VH-(optional Hinge)-CH2-CH3-3′) and are devoid of a light chain. The antibodies described herein may be oligoclonal, polyclonal, monoclonal (including full-length monoclonal antibodies), camelised, chimeric, CDR-grafted, multi-specific, bi-specific (including dual-binding antibodies), catalytic, chimeric, humanized, fully human, anti-idiotypic, including antibodies that can be labelled in soluble or bound form as well as fragments, variants or derivatives thereof, either alone or in combination with other amino acid sequences provided by known techniques. An antibody may be from any species. Antibodies described herein can be naked or conjugated to other molecules such as toxins, radioisotopes, etc.

The term “antigen binding domain,” “antigen binding region,” “antigen binding fragment,” and similar terms refer to that portion of an antibody which comprises the amino acid residues that interact with an antigen and confer on the binding agent its specificity and affinity for the antigen (e.g. the complementarity determining regions (CDRs)). The antigen binding region can be derived from any animal species, such as rodents (e.g. rabbit, rat or hamster) and humans. Preferably, the antigen binding region will be of human origin.

Antigen binding fragments described herein can include single-chain Fvs (scFv), single-chain antibodies, single domain antibodies, domain antibodies, Fv fragments, Fab fragments, F(ab′) fragments, F(ab′)2 fragments, antibody fragments that exhibit the desired biological activity, disulfide-stabilised variable region (dsFv), dimeric variable region (diabody), anti-idiotypic (anti-Id) antibodies (including, e.g. anti-Id antibodies to antibodies), intrabodies, linear antibodies, single-chain antibody molecules and multispecific antibodies formed from antibody fragments and epitope-binding fragments of any of the above. In particular, antibodies and antibody fragments described herein can include immunoglobulin molecules and immunologically active fragments of immunoglobulin molecules, i.e., molecules that contain an antigen-binding site. Digestion of antibodies with the enzyme, papain, results in two identical antigen-binding fragments, known also as “Fab” fragments, and a “Fc” fragment, having no antigen-binding activity but having the ability to crystallize. “Fab” when used herein refers to a fragment of an antibody that includes one constant and one variable domain of each of the heavy and light chains. The term “Fc region” herein is used to define a C-terminal region of an immunoglobulin heavy chain, including native-sequence Fc regions and variant Fc regions. The “Fc fragment” refers to the carboxy-terminal portions of both H chains held together by disulfides. The effector functions of antibodies are determined by sequences in the Fc region, the region which is also recognized by Fc receptors (FcR) found on certain types of cells. Digestion of antibodies with the enzyme, pepsin, results in a F(ab′)2 fragment in which the two arms of the antibody molecule remain linked and comprise two-antigen binding sites. The F(ab′)2 fragment has the ability to crosslink antigen.

“Fv” when used herein refers to the minimum fragment of an antibody that retains both antigen-recognition and antigen-binding sites. This region consists of a dimer of one heavy and one light chain variable domain in tight, non-covalent or covalent association. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The term “monoclonal antibody” as used herein refers to an antibody obtained from a population of substantially homogeneous antibodies, i.e. the individual antibodies comprising the population are identical except for possible naturally occurring mutations and/or post-translation modifications (e.g. isomerizations, amidations) that may be present in minor amounts. Monoclonal antibodies are highly specific, and are directed against a single antigenic determinant or epitope. In contrast, polyclonal antibody preparations typically include different antibodies directed against different antigenic determinants (or epitopes). The term “monoclonal antibody” as used herein encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal antibody” refers to such antibodies made in any number of ways including, but not limited to, hybridoma, phage selection, recombinant expression, and transgenic animals. The monoclonal antibodies herein can include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies that exhibit the desired biological activity.

The term “humanized antibody” refers to a subset of chimeric antibodies in which a “hypervariable region” from a non-human immunoglobulin (the donor antibody) replaces residues from a hypervariable region in a human immunoglobulin (recipient antibody). In general, a humanized antibody will include substantially all of at least one, and typically two, variable domains, in which all or substantially all of the hypervariable loops correspond to those of a non-human immunoglobulin sequence, and all or substantially all of the framework regions are those of a human immunoglobulin sequence, although the framework regions may include one or more substitutions that improve antibody performance, such as binding affinity, isomerization, immunogenicity, etc.

The term “bispecific antibody” means an antibody which comprises specificity for two target molecules, and includes, but is not limited to, formats such as DVD-Ig (see DiGiammarino et al., “Design and generation of DVD-Ig™ molecules for dual-specific targeting”, Meth. Mo. Biol., 2012, 889, 145-156), mAb2 (see WO2008/003103, the description of the mAb2 format is incorporated herein by reference), FIT-Ig (see WO2015/103072, the description of the FIT-Ig scaffold is incorporated herein by reference), mAb-dAb, dock and lock, Fab-arm exchange, SEEDbody, Triomab, LUZ-Y, Fcab, r,-body, orthogonal Fab, scDiabody-Fc, diabody-Fc, tandem scFv-Fc, Fab-scFv-Fc, Fab-scFv, intrabody, BiTE, diabody, DART, TandAb, scDiabody, scDiabody-CH3, Diabody-CH3, Triple body, Miniantibody, minibody, TriBi minibody, scFv-CH3 KIH, scFv-CH-CL-scFv, F(ab′)2-scFv, scFv-KIH, Fab-scFv-Fc, tetravalent HCab, ImmTAC, knobs-in-holes, knobs-in-holes with common light chain, knobs-in-holes with common light chain and charge pairs, charge pairs, charge pairs with common light chain, DT-IgG, DutaMab, IgG(H)-scFv, scFv-(H)IgG, IgG(L)-scFv, scFv-(L)IgG, IgG(L,H)-Fv, IgG(H)-V, V(H)—IgG, IgG(L)-V, V(L)-IgG, KIH IgG-scFab, 2 scFv-IgG, IgG-2 scFv, scFv4-Ig and zybody. For a review of bispecific formats, see Spiess, C., et al., Mol. Immunol. (2015). In another example, the bispecific molecule comprises an antibody which is fused to another non-Ig format, for example a T-cell receptor binding domain; an immunoglobulin superfamily domain; an agnathan variable lymphocyte receptor; a fibronectin domain (e.g. an Adnectin™); an antibody constant domain (e.g. a CH3 domain, e.g., a CH2 and/or CH3 of an Fcab™) wherein the constant domain is not a functional CH1 domain; an scFv; an (scFv)2; an sc-diabody; an scFab; a centyrin and an epitope binding domain derived from a scaffold selected from CTLA-4 (Evibody™); a lipocalin domain; Protein A such as Z-domain of Protein A (e.g. an Affibody™ or SpA); an A-domain (e.g. an Avimer™ or Maxibody™); a heat shock protein (such as and epitope binding domain derived from GroEI and GroES); a transferrin domain (e.g. a trans-body); ankyrin repeat protein (e.g. a DARPin™); peptide aptamer; C-type lectin domain (e.g. Tetranectin™); human γ-crystallin or human ubiquitin (an affilin); a PDZ domain; scorpion toxin; and a kunitz type domain of a human protease inhibitor.

In one example, the bispecific antibody is a mAb2. A mAb2 comprises a VH and VL domain from an intact antibody, fused to a modified constant region, which has been engineered to form an antigen-binding site, known as an “Fcab”. The technology behind the Fcab/mAb2 format is described in more detail in WO2008/003103, and the description of the mAb2 format is incorporated herein by reference.

In one example, a “bispecific antibody” does not include a FIT-Ig format. In one example, a “bispecific antibody” does not include a mAb2 format. In one example, a “bispecific antibody” does not include either a FIT-Ig format or a mAb2 format.

In another example, the bispecific antibody is a “dual binding antibody”. As used herein, the term “dual binding antibody” is a bispecific antibody wherein both antigen-binding domains are formed by a VH/VL pair, and includes FIT-Ig (see WO2015/103072, incorporated herein by reference), mAb-dAb, dock and lock, Fab-arm exchange, SEEDbody, Triomab, LUZ-Y, Fcab, d,-body, orthogonal Fab, scDiabody-Fc, diabody-Fc, tandem scFv-Fc, Fab-scFv-Fc, Fab-scFv, intrabody, BiTE, diabody, DART, TandAb, scDiabody, scDiabody-CH3, Diabody-CH3, Triple body, Miniantibody, minibody, scFv-CH3 KIH, scFv-CH-CL-scFv, F(ab′)2-scFv, scFv-KIH, Fab-scFv-Fc, tetravalent HCab, ImmTAC, knobs-in-holes, knobs-in-holes with common light chain, knobs-in-holes with common light chain and charge pairs, charge pairs, charge pairs with common light chain, DT-IgG, DutaMab, IgG(H)-scFv, scFv-(H)IgG, IgG(L)-scFv, scFv-(L)IgG, IgG(L,H)-Fv, IgG(H)-V, V(H)—IgG, IgG(L)-V, V(L)-IgG, KIH IgG-scFab, 2 scFv-IgG, IgG-2 scFv and scFv4-Ig.

The term “hypervariable region”, “CDR region” or “CDR” refers to the regions of an antibody variable domain which are hypervariable in sequence and/or form structurally defined loops. Generally, antigen binding sites of an antibody include six hypervariable regions: three in the VH (CDRH1, CDRH2, CDRH3), and three in the VL (CDRL1, CDRL2, CDRL3). These regions of the heavy and light chains of an antibody confer antigen-binding specificity to the antibody. CDRs may be defined according to the Kabat system (see Kabat, E. A.et al., 1991, “Sequences of Proteins of Immunological Interest”, 5th edit, NIH Publication no. 91-3242, U.S. Department of Health and Human Services). Other systems may be used to define CDRs, which as the system devised by Chothia et al (see Chothia, C. & Lesk, A. M., 1987, “Canonical structures for the hypervariable regions of immunoglobulins”, J. Mol. Biol., 196, 901-917) and the IMGT system (see Lefranc, M. P., 1997, “Unique database numbering system for immunogenetic analysis”, Immunol. Today, 18, 50). An antibody typically contains 3 heavy chain CDRs and 3 light chain CDRs. The term CDR or CDRs is used here to indicate one or several of these regions. A person skilled in the art is able to readily compare the different systems of nomenclature and determine whether a particular sequence may be defined as a CDR.

A “human antibody” is an antibody that possesses an amino-acid sequence corresponding to that of an antibody produced by a human and/or has been made using any of the techniques for making human antibodies and specifically excludes a humanized antibody comprising non-human antigen-binding residues. The term “specifically binds to” refers to measurable and reproducible interactions such as binding between a target and an antibody, which is determinative of the presence of the target in the presence of a heterogeneous population of molecules including biological molecules. For example, an antibody that specifically binds to a target (which can be an epitope) is an antibody that binds this target with greater affinity, avidity, more readily, and/or with greater duration than it binds to other targets. In one example, the extent of binding of an antibody to an unrelated target is less than about 10% of the binding of the antibody to the target as measured, e.g. by a radioimmunoassay (RIA).

An antibody that specifically binds to a SARS-CoV-2 spike protein antigen may be cross-reactive with related antigens such as those of other epidemic human coronaviruses like SARS and MERS. An antibody that specifically binds to a SARS-CoV-2 spike protein antigen can be identified, for example, by immunoassays, BIAcore™, or other techniques known to those of skill in the art. An antibody binds specifically to a SARS-CoV-2 spike protein antigen when it binds to a SARS-CoV-2 spike protein antigen with higher affinity than to any cross-reactive antigen as determined using experimental techniques, such as radioimmunoassays (RIA) and enzyme-linked immunosorbent assays (ELISAs). Typically, a specific or selective reaction will be at least twice background signal or noise and more typically more than 10 times (such as more than 15 times, more than 20 times, more than 50 times or more than 100 times) background. See, e.g. Paul, ed., 1989, Fundamental Immunology Second Edition, Raven Press, New York at pages 332-336 for a discussion regarding antibody specificity.

As used herein, “authorization number” or “marketing authorization number” refers to a number issued by a regulatory agency upon that agency determining that a particular medical product and/or composition may be marketed and/or offered for sale in the area under the agency's jurisdiction. As used herein “regulatory agency” refers to one of the agencies responsible for evaluating, e.g. the safety and efficacy of a medical product and/or composition and controlling the sales/marketing of such products and/or compositions in a given area. The Food and Drug Administration (FDA) in the US and the European Medicines Agency (EPA) in Europe are but two examples of such regulatory agencies. Other non-limiting examples can include SDA, MPA, MHPRA, IMA, ANMAT, Hong Kong Department of Health-Drug Office, CDSCO, Medsafe, and KFDA.

As used herein, the term “carrier” refers to a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions.

As used herein, the term “composition” is intended to encompass a product containing the specified ingredients (e.g. an antibody) in, optionally, the specified amounts, as well as any product which results, directly or indirectly, from combination of the specified ingredients in, optionally, the specified amounts.

As used herein the term “comprising” or “comprises” is used with reference to antibodies, uses, compositions, methods, and respective component(s) thereof, that are essential to the method or composition, yet open to the inclusion of unspecified elements, whether essential or not.

The term “consisting of” refers to antibodies, uses, compositions, methods, and respective components thereof as described herein, which are exclusive of any element not recited in that description of the example.

As used herein the term “consisting essentially of” refers to those elements required for a given example. The term permits the presence of elements that do not materially affect the basic and novel or functional characteristic(s) of that example.

The term “effector function” as used herein is meant to refer to one or more of antibody dependant cell mediated cytotoxic activity (ADCC), complement-dependant cytotoxic activity (CDC) mediated responses, Fc-mediated phagocytosis or antibody dependant cellular phagocytosis (ADCP), antibody recycling via the FcRn receptor, opsonisation of the virus particle and complement-mediated disruption of virus particle lipid envelope.

An “effective amount” refers to an amount effective, at dosages and for periods of time necessary, to achieve the desired effect, including atherapeutic or prophylactic result. A “therapeutically effective amount” refers to the minimum concentration required to effect a measurable improvement or prevention of a particular disorder. A therapeutically effective amount herein may vary according to factors such as the disease state, age, sex, and weight of the patient, and the ability of the antibody to elicit a desired response in the individual. A therapeutically effective amount is also one in which toxic or detrimental effects of the antibody are outweighed by the therapeutically beneficial effects. A “prophylactically effective amount” refers to an amount effective, at the dosages and for periods of time necessary, to achieve the desired prophylactic result. In some examples, the effective amount of an antibody is from about 0.1 mg/kg (mg of antibody per kg weight of the subject) to about 100 mg/kg. In certain examples, an effective amount of an antibody provided therein is about 0.1 mg/kg, about 0.5 mg/kg, about 1 mg/kg, 3 mg/kg, 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg about 90 mg/kg or about 100 mg/kg (or a range therein). In some examples, “effective amount” as used herein also refers to the amount of an antibody to achieve a specified result (e.g. neutralising the SARS-CoV-2 spike protein). The term “epitope” as used herein refers to a localized region on the surface of an antigen, such as SARS-CoV-2 spike protein, that is capable of being bound to one or more antigen binding regions of an antibody, and that has antigenic or immunogenic activity in an animal, preferably a mammal, and most preferably in a human, that is capable of eliciting an immune response. An epitope having immunogenic activity is a portion of a polypeptide that elicits an antibody response in an animal. An epitope having antigenic activity is a portion of a polypeptide to which an antibody specifically binds as determined by any method well known in the art, for example, by the immunoassays described herein. Antigenic epitopes need not necessarily be immunogenic. Epitopes usually consist of chemically active surface groupings of molecules such as amino acids or sugar side chains and have specific three-dimensional structural characteristics as well as specific charge characteristics. A region of a polypeptide contributing to an epitope may be contiguous amino acids of the polypeptide or the epitope may come together from two or more non-contiguous regions of the polypeptide. The epitope may or may not be a three-dimensional surface feature of the antigen. In certain embodiments, a SARS-CoV-2 spike protein epitope is athree-dimensional surface feature of a SARS-CoV-2 spike protein polypeptide (e.g. in a trimeric form of a SARS-CoV-2 spike protein polypeptide). In other embodiments, a SARS-CoV-2 spike protein epitope is linear feature of a SARS-CoV-2 spike protein polypeptide (e.g. in a trimeric form or monomeric form of the SARS-CoV-2 spike protein polypeptide). Antibodies provided herein may specifically bind to an epitope of the monomeric (denatured) form of SARS-CoV-2 spike protein, an epitope of the trimeric (native) form of SARS-CoV-2 spike protein, or both the monomeric (denatured) form and the trimeric (native) form of SARS-CoV-2 spike protein. In specific examples, the antibodies provided herein specifically bind to an epitope of the trimeric form of SARS-CoV-2 spike protein but do not specifically bind the monomeric form of SARS-CoV-2 spike protein. In certain embodiments, antibodies provided herein may specifically bind to an epitope of the SARS-CoV-2 spike protein in part by using an interaction with an N-linked glycan or another post-translational modification of the spike protein. Binding to the respective epitope thus might involve moving the N-linked glycan or post-translational modification away thereby removing or reducing steric hindrance that would otherwise prevent or hinder antibody binding. In certain embodiments, antibodies provided herein may specifically bind to an epitope of the SARS-CoV-2 spike protein that only arises following priming cleavage between S1 and S2 or following activating cleavage at the S2′ cleavage site. Antibodies may be provided which bind to the same epitope as any antibody disclosed herein. Antibodies may be provided which bind to the same epitope as an IMPI antibody disclosed herein. Antibodies may be provided which bind to the same epitope as a YANG antibody disclosed herein. This is optionally determined using X-ray crystallography or other fine mapping techniques such as electron microscopy to identify the contact points between antibody and antigen. An antibody may contact the SARS-CoV-2 spike protein with a footprint that fully or partly overlaps with that of an antibody disclosed herein. An antibody may contact the SARS-CoV-2 spike protein with a footprint that fully or partly overlaps with that of an IMPI antibody.

An antibody may contact the SARS-CoV-2 spike protein with a footprint that fully or partly overlaps with that of a YANG antibody. As described elsewhere herein, competition between antibodies may also be determined, for example using SPR, and antibodies of the present invention may compete for binding to the spike protein (compete for binding to their epitope) with an IgG antibody that is any IMPI antibody or YANG antibody described herein.

The term “excipients” as used herein refers to inert substances which are commonly used as a diluent, vehicle, preservatives, binders, or stabilizing agent for drugs and includes, but not limited to, proteins (e.g. serum albumin, etc.), amino acids (e.g. aspartic acid, glutamic acid, lysine, arginine, glycine, histidine, etc.), fatty acids and phospholipids (e.g. alkyl sulfonates, caprylate, etc.), surfactants (e.g. SDS, polysorbate, non-ionic surfactant, etc.), saccharides (e.g. sucrose, maltose, trehalose, etc.) and polyols (e.g. mannitol, sorbitol, etc.). See, also, Remington's Pharmaceutical Sciences (1990) Mack Publishing Co., Easton, Pa., which is hereby incorporated by reference in its entirety.

The term “fusion protein” as used herein refers to a polypeptide that comprises an amino acid sequence of an antibody and an amino acid sequence of a heterologous polypeptide or protein (i.e. a polypeptide or protein not normally a part of the antibody (e.g. a non-anti-SARS-CoV-2 spike protein antigen antibody)). The term “fusion” when used in relation to an antibody refers to the joining of a peptide or polypeptide, or fragment, variant and/or derivative thereof, with a heterologous peptide or polypeptide. Preferably, the fusion protein retains the biological activity of the anti-SARS-CoV-2 spike protein antibody.

The term “heavy chain” when used with reference to an antibody refers to five distinct types, called alpha (α), delta (δ), epsilon (a), gamma (γ) and mu (μ), based on the amino acid sequence of the heavy chain constant domain. These distinct types of heavy chains are well known and give rise to five classes of antibodies, IgA, IgD, IgE, IgG and IgM, respectively, including two subclasses of IgA, namely IgA1 and IgA2 and four subclasses of IgG, namely IgG1, IgG2, IgG3 and IgG4. Preferably the heavy chain is a human heavy chain. In the human population, multiple heavy chain constant region alleles, of each immunoglobulin or immunoglobulin subclass, exist. The nucleotide and amino acid sequences of these allelic variants are accessible on publicly available databases such as IMGT, ENSEMBL Swiss-Prot and Uniprot. Allelic variants may also be identified in various genome sequencing projects. In one example, the antibodies disclosed herein comprise a heavy chain encoded by a IgG1 constant region allele, which includes, but is not limited to, human IGHG1*01, IGHG1*02, IGHG1*03, IGHG1*04 and IGHG1*05 (the nucleotide and corresponding amino acid sequences of which are set out in Table 2). In one example, the antibodies disclosed herein comprise a protein encoded by a IgG4 constant region allele, which includes but is not limited to human IGHG4*01, IGHG4*02, IGHG4*03 and IGHG4*04 (the nucleotide and corresponding amino acid sequences of which are set out in Table 2). In another example, the heavy chain is an IgA isotype, human IgA1 or human IgA2, example amino acid sequences for which are shown in Table 2. In another example, the heavy chain is a disabled IgG isotype, e.g. a disabled IgG4. In certain examples, the antibodies comprise a human gamma 4 constant region. In another example, the heavy chain constant region does not bind Fc-γ receptors, and e.g. comprises a Leu235Glu mutation. In another example, the heavy chain constant region comprises a Ser228Pro mutation to increase stability. In another example, the heavy chain constant region is IgG4-PE. In another example, the antibodies disclosed herein comprise a heavy chain constant region encoded by a murine IgG1 constant region allele, which includes but is not limited to mouse IGHG1*01 or IGHG1*02.

The term “host” as used herein refers to an animal, preferably a mammal, and most preferably a human. The term “host cell” as used herein refers to the particular subject cell transfected with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

The term “in combination” in the context of the administration of other therapies refers to the use of more than one therapy. The use of the term “in combination” does not restrict the order in which therapies are administered to a subject with a disease. A first therapy can be administered before (e.g. 1 minute, 45 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks), concurrently, or after (e.g. 1 minute, 45 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks) the administration of a second therapy to a subject. Any additional therapy can be administered in any order with the other additional therapies. In certain examples, the antibodies can be administered in combination with one or more therapies.

As used herein, “injection device” refers to a device that is designed for carrying out injections, an injection including the steps of temporarily fluidically coupling the injection device to a person's tissue, typically the subcutaneous tissue. An injection further includes administering an amount of liquid drug into the tissue and decoupling or removing the injection device from the tissue. In some examples, an injection device can be an intravenous device or IV device, which is a type of injection device used when the target tissue is the blood within the circulatory system, e.g. the blood in a vein. A common, but non-limiting example of an injection device is a needle and syringe.

As used herein, “instructions” refers to a display of written, printed or graphic matter on the immediate container of an article, for example the written material displayed on a vial containing a pharmaceutically active agent, or details on the composition and use of a product of interest included in a kit containing a composition of interest. Instructions set forth the method of the treatment as contemplated to be administered or performed.

An “isolated” or “purified” antibody or protein is one that has been identified, separated and/or recovered from a component of its production environment (e.g. natural or recombinant). For example, the antibody or protein is substantially free of cellular material or other contaminating proteins from the cell or tissue source from which the antibody is derived, or substantially free of chemical precursors or other chemicals when chemically synthesized. The language “substantially free of cellular material” includes preparations of an antibody in which the antibody is separated from cellular components of the cells from which it is isolated or recombinantly produced. Thus, an antibody that is substantially free of cellular material includes preparations of antibody having less than about 30%, 20%, 10%, or 5% (by dry weight) of heterologous protein (also referred to herein as a “contaminating protein”). When the antibody is recombinantly produced, it is also preferably substantially free of culture medium, i.e. culture medium represents less than about 20%, 10%, or 5% of the volume of the protein preparation. When the antibody is produced by chemical synthesis, it is preferably substantially free of chemical precursors or other chemicals, i.e., it is separated from chemical precursors or other chemicals which are involved in the synthesis of the protein. Accordingly, such preparations of the antibody have less than about 30%, 20%, 10%, 5% (by dry weight) of chemical precursors or compounds other than the antibody of interest. In a preferred example, antibodies are isolated or purified.

The terms “Kabat numbering,” and like terms are recognized in the art and refer to a system of numbering amino acid residues which are more variable (i.e. hypervariable) than other amino acid residues in the heavy chain variable regions of an antibody, or an antigen binding portion thereof (Kabat et al., (1971) Ann. NY Acad. Sci., 190:382-391 and, Kabat et al. (1991) Sequences of Proteins of Immunological Interest, Fifth Edition, U.S. Department of Health and Human Services, NIH Publication No. 91-3242). For the heavy chain variable region, the hypervariable region typically ranges from amino acid positions 31 to 35 for CDR1, amino acid positions 50 to 65 for CDR2, and amino acid positions 95 to 102 for CDR3.

“Label” or “labelled” as used herein refers to the addition of a detectable moiety to a polypeptide, for example, a radiolabel, fluorescent label, enzymatic label, chemiluminescent label or a biotinyl group or gold. Radioisotopes or radionuclides may include 3H, 14C, 15N, 35S, 90Y, 99Tc, 115In, 1251, 1311, fluorescent labels may include rhodamine, lanthanide phosphors or FITC and enzymatic labels may include horseradish peroxidase, β-galactosidase, luciferase, alkaline phosphatase. Additional labels include, by way of illustration and not limitation: enzymes, such as glucose-6-phosphate dehydrogenase (“G6PDH”), alpha-D-galactosidase, glucose oxydase, glucose amylase, carbonic anhydrase, acetylcholinesterase, lysozyme, malate dehydrogenase and peroxidase; dyes (e.g. cyanine dyes, e.g. Cy5TM, Cy5.5TM. or Cy7TM); additional fluorescent labels or fluorescers include, such as fluorescein and its derivatives, fluorochrome, GFP (GFP for “Green Fluorescent Protein”), other fluorescent proteins (e.g. mCherry, mTomato), dansyl, umbelliferone, phycoerythrin, phycocyanin, allophycocyanin, o-phthaldehyde, and fiuorescamine; fluorophores such as lanthanide cryptates and chelates e.g. Europium etc (Perkin Elmer and Cisbio Assays); chemoluminescent labels or chemiluminescers, such as isoluminol, luminol and the dioxetanes; sensitisers; coenzymes; enzyme substrates; particles, such as latex or carbon particles; metal sol; crystallite; liposomes; cells, etc., which may be further labelled with a dye, catalyst or other detectable group; molecules such as biotin, digoxygenin or 5-bromodeoxyuridine; toxin moieties, such as for example a toxin moiety selected from a group of Pseudomonas exotoxin (PE or a cytotoxic fragment or mutant thereof), Diptheria toxin or a cytotoxic fragment or mutant thereof, a botulinum toxin A, B, C, D, E or F, ricin or a cytotoxic fragment thereof e.g. ricin A, abrin or a cytotoxic fragment thereof, saporin or a cytotoxic fragment thereof, pokeweed antiviral toxin or a cytotoxic fragment thereof and bryodin 1 or a cytotoxic fragment thereof.

The term “light chain” when used in reference to an antibody refers to the immunoglobulin light chains, of which there are two types in mammals, lambda (λ) and kappa (κ). Preferably, the light chain is a human light chain. Preferably the light chain constant region is a human constant region. In the human population, multiple light chain constant region alleles exist. The nucleotide and amino acid sequences of these allelic variants are accessible on publicly available databases such as IMGT, ENSEMBL, Swiss-Prot and Uniprot. In one example, the antibodies disclosed herein comprise a protein encoded by a human K constant region allele, which includes, but is not limited to, IGKC*01, IGKC*02, IGKC*03, IGKC*04 and IGKC*05 (the nucleotide and corresponding amino acid sequences of which are set out in Table 2). In one example, the antibodies disclosed herein comprise a protein encoded by a human, constant region allele, which includes but is not limited to IGLC1*01, IGLC1*02, IGLC2*01, IGLC2*02, IGLC2*03, IGLC3*01, IGLC3*02, IGLC3*03, IGLC3*04, IGLC6*01, IGLC7*01, IGLC7*02, and IGLC7*03 (the nucleotide and corresponding amino acid sequences of which are set out in Table 2). In another example, the antibodies disclosed herein comprise a light chain constant region encoded by a mouse K constant region allele, which includes, but is not limited to, IGKC*01, IGKC*03 or IGKC*03 (the nucleotide and corresponding amino acid sequences of which are set out in Table 2). In another example, the antibodies disclosed herein comprise a light chain constant region encoded by a mouse a constant region allele, which includes, but is not limited to, IGLC1*01, IGLC2*01 or IGLC3*01 (the nucleotide and corresponding amino acid sequences of which are set out in Table 2).

“Percent (%) amino acid sequence identity” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEG ALIGN™ (DNASTAR) software. In one example, the % identity is about 70%. In one example, the % identity is about 75%. In one example, the % identity is about 80%. In one example, the % identity is about 85%. In one example, the % identity is about 90%. In one example, the % identity is about 92%. In one example, the % identity is about 95%. In one example, the % identity is about 97%. In one example, the % identity is about 98%. In one example, the % identity is about 99%. In one example, the % identity is 100%.

The term “naturally occurring” or “native” when used in connection with biological materials such as nucleic acid molecules, polypeptides, host cells, and the like, refers to those which are found in nature and not manipulated by a human being.

As used herein, “packaging” refers to how the components are organized and/or restrained into a unit fit for distribution and/or use. Packaging can include, e.g. boxes, bags, syringes, ampoules, vials, tubes, clamshell packaging, barriers and/or containers to maintain sterility, labelling, etc.

The term “pharmaceutically acceptable” as used herein means being approved by a regulatory agency of the Federal or a state government, or listed in the U.S. Pharmacopeia, European Pharmacopeia or other generally recognized Pharmacopeia for use in animals, and more particularly in humans.

As used herein, the term “polynucleotide,” “nucleotide,” nucleic acid” “nucleic acid molecule” and other similar terms are used interchangeable and include DNA, RNA, mRNA and the like.

As used herein, the terms “prevent”, “preventing”, and “prevention” refer to the total or partial inhibition of the development, recurrence, onset or spread of a SARS-CoV-2 related disease, such as COVID-19,and/or symptom related thereto, resulting from the administration of a therapy or combination of therapies provided herein (e.g. a combination of prophylactic or therapeutic agents, such as an antibody).

The term “soluble” refers to a polypeptide that is lacking one or more transmembrane or cytoplasmic domains found in the native or membrane-associated form. In one example, the “soluble” form of a polypeptide lacks both the transmembrane domain and the cytoplasmic domain.

The term “subject” or “patient” refers to any animal, including, but not limited to, mammals. As used herein, the term “mammal” refers to any vertebrate animal that suckle their young and either give birth to living young (eutharian or placental mammals) or are egg-laying (metatharian or nonplacental mammals). Examples of mammalian species include, but are not limited to, humans and other primates, including non-human primates such as chimpanzees and other apes and monkey species; farm animals such as cattle, sheep, pigs, goats and horses; domestic mammals such as dogs and cats; laboratory animals including rodents such as mice, rats (including cotton rats) and guinea pigs; birds, including domestic, wild and game birds such as chickens, turkeys and other gallinaceous birds, ducks, geese, and the like.

As used herein “substantially all” refers to refers to at least about 60%, at least about 70%, at least about 75%, at least about 80%, at least about 85%, at least about 90%, at least about 95%, at least about 98%, at least about 99%, or about 100%.

The term “surfactant” as used herein refers to organic substances having amphipathic structures; namely, they are composed of groups of opposing solubility tendencies, typically an oil-soluble hydrocarbon chain and a water-soluble ionic group. Surfactants can be classified, depending on the charge of the surface-active moiety, into anionic, cationic, and non-ionic surfactants. Surfactants are often used as wetting, emulsifying, solubilizing, and dispersing agents for various pharmaceutical compositions and preparations of biological materials.

As used herein, the term “tag” refers to any type of moiety that is attached to, e.g. a polypeptide and/or a polynucleotide that encodes a SARS-CoV-2 antibody. For example, a polynucleotide that encodes a SARS-CoV-2 antibody can contain one or more additional tag-encoding nucleotide sequences that encode e.g. a detectable moiety or a moiety that aids in affinity purification. When translated, the tag and the antibody can be in the form of a fusion protein. The term “detectable” or “detection” with reference to a tag refers to any tag that is capable of being visualized or wherein the presence of the tag is otherwise able to be determined and/or measured (e.g. by quantitation). A non-limiting example of a detectable tag is a fluorescent tag.

As used herein, the term “therapeutic agent” refers to any agent that can be used in the treatment, management or amelioration of a SARS-CoV-2-related disease or condition, such as COVID-19 and/or a symptom related thereto. In certain examples, the term “therapeutic agent” refers to an antibody. In certain other examples, the term “therapeutic agent” refers to an agent other than an antibody. Preferably, a therapeutic agent is an agent which is known to be useful for, or has been or is currently being used for the treatment, management or amelioration of a SARS-CoV-2-related disease or condition, such as COVID-19 and/or one or more symptoms related thereto. In specific examples, the therapeutic agent is an anti-SARS-CoV-2 antibody. In specific examples, the therapeutic agent is a fully human anti-SARS-CoV-2 antibody, such as a fully human SARS-CoV-2 monoclonal antibody.

As used herein, the term “therapy” refers to any protocol, method and/or agent that can be used in the prevention, management, treatment and/or amelioration of a SARS-CoV-2-related disease or condition, such as COVID-19. In certain examples, the terms “therapies” and “therapy” refer to a biological therapy, supportive therapy, and/or other therapies useful in the prevention, management, treatment and/or amelioration of a SARS-CoV-2-related disease or condition, such as COVID-19 known to one of skill in the art such as medical personnel.

The terms “treat”, “treatment” and “treating” refer to the reduction or amelioration of the progression, seventy, and/or duration of a SARS-CoV-2-related disease or condition, such as COVID-19 resulting from the administration of one or more therapies (including, but not limited to, the administration of one or more prophylactic or therapeutic agents, such as an antibody). In specific examples, such terms refer to the reduction or inhibition of the binding of SARS-CoV-2 to ACE 2, and/or the inhibition or reduction of one or more symptoms associated with a SARS-CoV-2-related disease or condition, such as COVID-19.

The term “variable region” or “variable domain” refers to a portion of the light and heavy chains, typically about the amino-terminal 120 to 130 amino acids in the heavy chain and about 100 to 110 amino acids in the light chain, which differ extensively in sequence among antibodies and are used in the binding and specificity of each particular antibody for its particular antigen. The variability in sequence is concentrated in those regions called complimentarily determining regions (CDRs) while the more highly conserved regions in the variable domain are called framework regions (FR). The CDRs are primarily responsible for the interaction of the antibody with antigen. Numbering of amino acid positions used herein is according to IMGT (Lefranc MP “IMGT unique numbering for immunoglobulin and T cell receptor variable domains and Ig superfamily V-like domains”, Dev. Comp. Immunol. 27(1):55-77 (2003)). In preferred examples, the variable region is a human variable region.

Definitions of common terms in cell biology and molecular biology can be found in “The Merck Manual of Diagnosis and Therapy”, 19th Edition, published by Merck Research Laboratories, 2006 (ISBN 0-911910-19-0); Robert S. Porter et al. (eds.), The Encyclopedia of Molecular Biology, published by Blackwell Science Ltd., 1994 (ISBN 0-632-02182-9); Benjamin Lewin, Genes X, published by Jones & Bartlett Publishing, 2009 (ISBN-10: 0763766321); Kendrew et al. (Eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

Unless otherwise stated, the present disclosure was performed using standard procedures, as described, for example in Sambrook et al., Molecular Cloning: A Laboratory Manual (4 ed.), Cold Spring Harbor Laboratory Press, Cold Spring Harbor, N.Y., USA (2012); Davis et al., Basic Methods in Molecular Biology, Elsevier Science Publishing, Inc., New York, USA (1995); or Methods in Enzymology: Guide to Molecular Cloning Techniques Vol. 152, S. L. Berger and A. R. Kimmel Eds., Academic Press Inc., San Diego, USA (1987); Current Protocols in Protein Science (CPPS) (John E. Coligan, et al., ed., John Wiley and Sons, Inc.), Current Protocols in Cell Biology (CPCB) (Juan S. Bonifacino et al. ed., John Wiley and Sons, Inc.), and Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998) which are all incorporated by reference herein in their entireties.

Other terms are defined herein within the description of the various examples of the disclosure.

### Anti-SARS-COV-2 Antibodies

The antibodies described herein are described with respect to the following concepts, aspects, sentences, arrangements and embodiments. Unless otherwise stated, all concepts, embodiments, sentences, arrangements and aspects are to be read as being able to be combined with any other concept, aspect, sentence, arrangement or embodiment, unless such combination would not make technical sense or is explicitly stated otherwise.

### Binding—Location:

Antibodies that specifically bind the spike protein of SARS-CoV-2 are provided. In particular, neutralising antibodies, which inhibit or prevent SARS-CoV-2 from entering cells are provided. In some aspects the antibodies specifically bind the S 1 subunit of the SARS-CoV-2 spike protein. For example, antibodies may bind the receptor binding domain (RBD) of the S1 subunit of the SARS-CoV-2 spike protein. Such antibodies binding the RBD may or may not compete with ACE2 for binding to SARS-Cov-2 and thus may or may not directly inhibit binding of SARS-CoV-2 to its receptor ACE2. Alternatively, the antibodies may preferentially bind to the trimer form of the SARS-CoV-2 spike protein. Alternatively, the antibodies may specifically bind the S2 subunit of the SARS-CoV-2 spike protein.

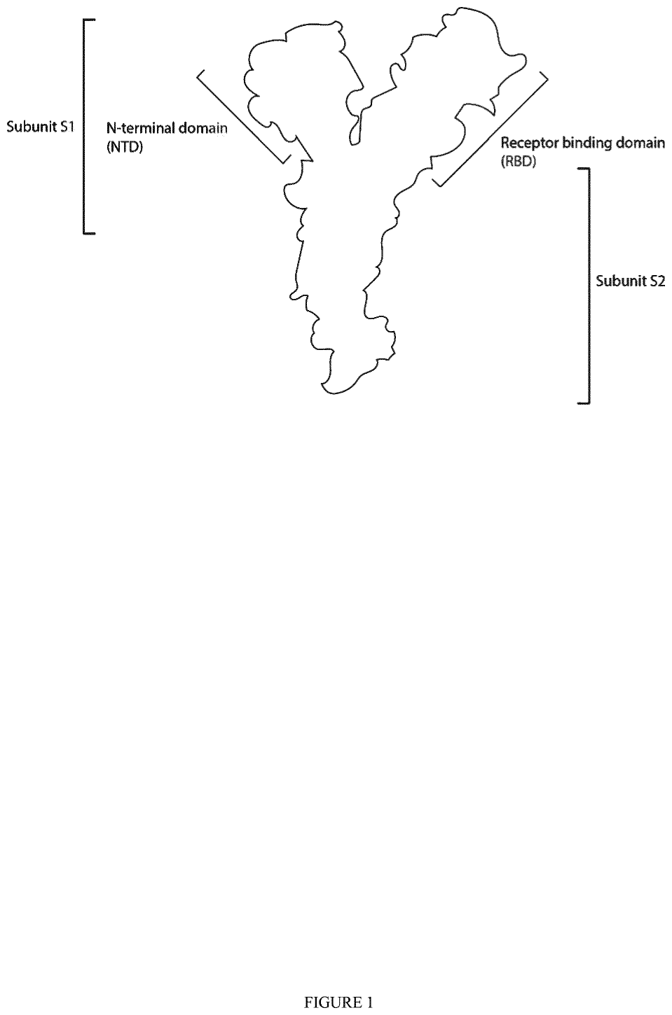

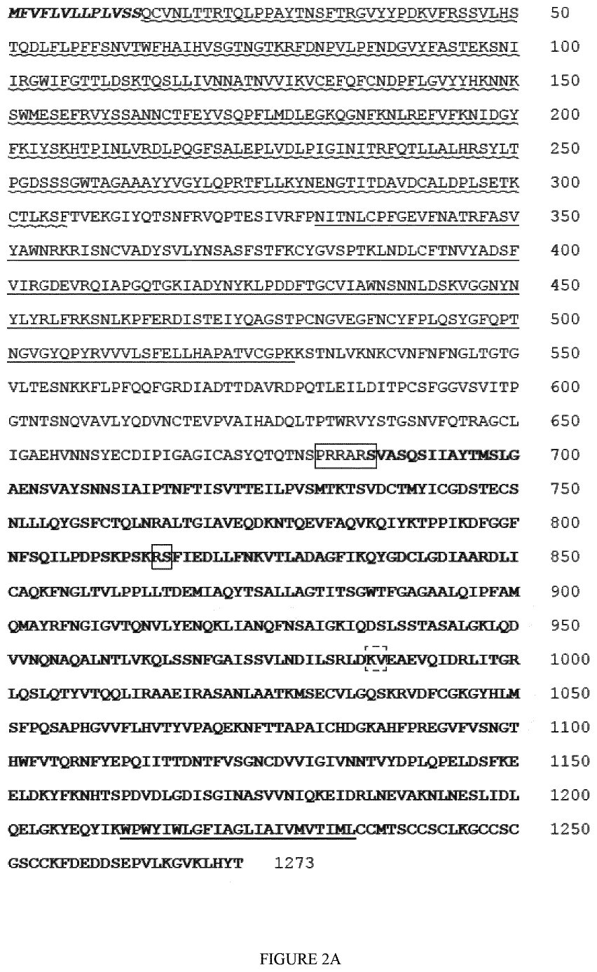

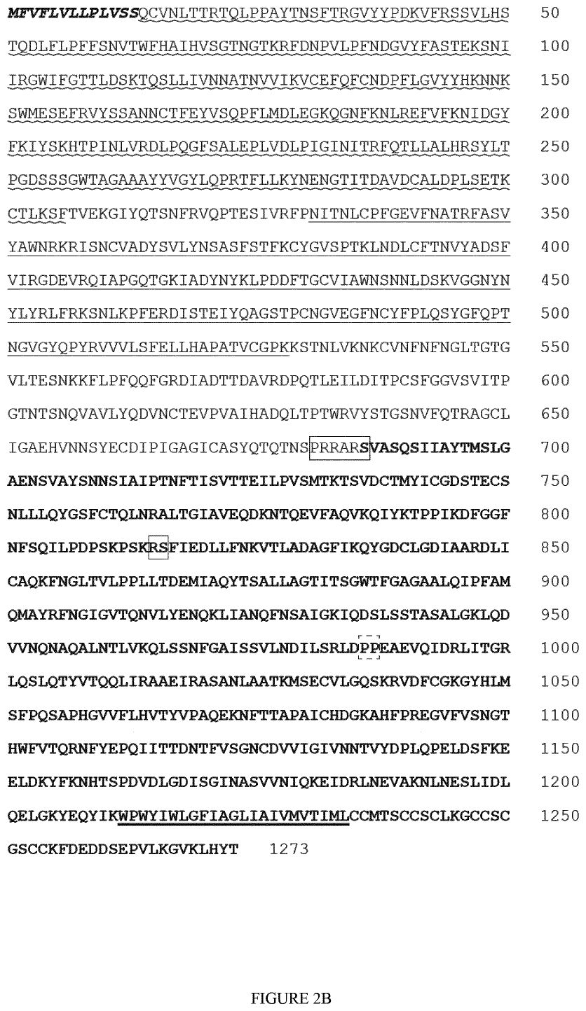

The spike protein and its domain and subunit structure are illustrated in FIG. 1, FIG. 2A and FIG. 2B, and reference herein to the S1 subunit, S2 subunit, RBD, NTD, extracellular domain and trimer refer to the wild-type spike protein in FIG. 2A unless stated otherwise or unless indicated by context. It will be appreciated that, as the epidemic has spread, vast numbers of different strains of SARS-CoV-2, comprising a variety of mutations, are now at large in the population and that these include spike proteins with a number of mutations relative to the defined wild-type of FIG. 2A. One such mutation is D614G (i.e., substitution of glycine for aspartic acid at residue 614 of the spike protein) which is now present in the majority of clinical isolates of SARS-CoV-2. Preferably, antibodies ofthe present invention bind SARS-CoV-2 D614G with at least the affinity with which they bind SARS-CoV-2614D. This residue lies between the RBD and S2 domains and is thus not present in soluble preparations of these domains, but anti-RBD and anti-S2 antibodies may be tested for binding to the spike protein trimer to confirm maintenance of binding to the D614G form. Similarly, neutralisation assays may be performed with SARS-CoV-2 spike D614G to confirm neutralising potency.

An antibody of the present invention may be one which competes for binding to the isolated soluble RBD subunit with any anti-RBD IMPI antibody described herein, such as IMPI-059, IMPI-017 or IMPI-004.

An antibody of the present invention may be one which competes for binding to the isolated soluble RBD subunit with any anti-RBD YANG antibody described herein, such as YANG-1112, YANG-2107, YANG-2108, YANG-2111, and YANG-1401.

An antibody of the present invention may be one which competes for binding to the isolated soluble S2 subunit with any anti-S2 IMPI antibody described herein, such as IMPI-013.

An antibody of the present invention may be one which competes for binding to the isolated soluble S2 subunit with any anti-S2 YANG antibody described herein, such as YANG-2203, YANG-2204, YANG-2205, YANG-2206, YANG-2207, or YANG-2208.

An antibody of the present invention may be one which competes for binding to the isolated soluble S2 subunit with any anti-NTD YANG antibody described herein, YANG-1301, YANG-1302, YANG-1303, YANG-1304, YANG-1305; YANG-2301, YANG-2302, YANG-2303, YANG-2304, YANG-2305, or YANG-2306.

An antibody of the present invention may be one which competes for binding to the SARS-CoV-2 spike protein trimer with any IMPI antibody described herein, optionally with a “trimer-only” binding antibody.

Methods of determining competition between molecules are described elsewhere herein (e.g., SPR) and may be performed with the test antibody and IMPI antibody in IgG format or optionally in scFv format.

Methods of determining competition between molecules are described elsewhere herein (e.g., SPR) and may be performed with the test antibody and YANG antibody in IgG format or optionally in scFv format.

### Binding—Measurement:

Any suitable method may be used to determine whether an antibody binds to the SARS-CoV-2 spike protein. Such a method may comprise surface plasmon resonance (SPR), bio-layer interferometry, or an ELISA to determine specificity of antibodies. An antibody may be said to bind its antigen if the level of binding to antigen is at least 2.5 fold greater, e.g., at least 10 fold greater, than binding to a control antigen. Binding between an antibody and its cognate antigen is often referred to as specific binding. Precise identification of the residues bound by an antibody can usually be obtained using x-ray crystallography. This technique may be used to determine that an antibody described herein binds one or more residues of SARS-CoV-2 spike protein.

Ability of an antibody to bind its target antigen, and the specificity and affinity of that binding (KD, Kd and/or Ka) can be determined by any routine method in the art, e.g. using surface plasmon resonance (SPR), such as by Biacore™ (Cytiva Life Sciences) or using the ProteOn XPR36™ (Bio-Rad®), using KinExA® (Sapidyne Instruments, Inc), or using ForteBio Octet (Pall ForteBio Corp.).

The term “KD”, as used herein, is intended to refer to the equilibrium dissociation constant of a particular antibody-antigen interaction. Affinity of antibody-antigen binding may be determined, e.g., by SPR. Affinity may also be determined by bio-layer interferometry. Examples of affinity determination by SPR are provided in Example 6 herein. In some examples, an antibody may bind to a SARS-CoV-2 spike protein with an affinity (KD) of 1 mM or less, preferably less than 50 nM, less than 40 nM, less than 30 nM, less than 20 nM, as determined by SPR. In other examples, the antibody may bind to SARS-CoV-2 spike protein with a KD of less than 10 nM (e.g. less than 9 nM, less than 8 nM, less than 7 nM, less than 6 nM, less than 5 nM, less than 4 nM, less than 3 nM, less than 2 nM or less than 1 nM as determined by SPR. Preferably the KD may be less than 1 nM as determined by SPR. The KD may be 0.9 nM or less, 0.8 nM or less, 0.7 nM or less, 0.6 nM or less, 0.5 nM or less, 0.4 nM or less, 0.3 nM or less, 0.2 nM or less, or 0.1 nM or less, as determined by SPR. In some examples, an antibody may bind to a SARS-CoV-2 spike protein with KD of 0.1 nM or less, as determined by SPR. In some examples, an antibody may bind to a SARS-CoV-2 spike protein with a KD of 50 pM or less, as determined by SPR. Binding and binding affinity can be determined to various purified spike proteins and sub-domains, for example, the wild type trimer spike protein (FIG. 2A), the trimer stabilised by proline mutations (FIG. 2B), mutations to the trimeric spike protein observed in clinical isolates, for example D614G, expressed and purified sub-domains of the trimers, such as the S1 subunit or the S2 subunit, or the Receptor binding domain (RBD) or the N-terminal domain (NTD) or any of the above sub-domains with mutations observed from clinical isolates. If the antibody epitope is a linear continuous epitope, then binding and binding affinity can be determined using synthetic purified peptide sequences.

In one example, the antibody binds to the SARS-CoV-2 spike protein with an affinity of less than 1 nM (e.g. from 1 nM to 0.01 pM or from 1 nM to 0.1 pM, or from 1 nM to 1 pM), as determined by SPR. In one example, the antibody binds to the SARS-CoV-2 spike protein with an affinity of less than 10 nM (e.g. from 10 nM to 0.01 pM or from 10 nM to 0.1 pM, or from 10 nM to 1 pM), as determined by SPR. In one example, the antibody binds to the SARS-CoV-2 spike protein with an affinity of less than 0.1 nM (e.g. from 0.1 nM to 0.01 pM or from 0.1 nM to 0.1 pM, or from 0.1 nM to 1 pM), as determined by SPR. In one example, the antibody binds to SARS-CoV-2 spike protein with an affinity of less than 0.01 nM (e.g. from 0.011 nM to 0.01 pM or from 0.01 nM to 0.1 pM), as determined by SPR. In another example, the KD is within a range of 0.01 to 1 nM, or a range of 0.05 to 2 nM, or a range of 0.05 to lnM, as determined by SPR.

In one example, the SPR is carried out at 25° C. A suitable SPR protocol is set out in detail in Example 6.

In brief, the affinity of the antibody can be determined using SPR by:
1. Coupling mouse anti-human (or other relevant human, rat or non-human vertebrate antibody constant region species-matched) IgG to a biosensor chip (e.g. dextran-coated gold chip) such as by primary amine coupling. Thus, an anti-Fc antibody may be covalently immobilised on the chip surface using amine coupling.
2. Exposing the mouse anti-human IgG (or other matched species antibody) to the test antibody (e.g., in human IgG format) to capture the test antibody on the chip;
3. Passing the test antigen over the chip's capture surface at a series of concentrations up to a maximum of 100 nM, e.g., at 0.39, 1.56, 6.25, 25 and 100 nM, and a 0 nM (i.e. buffer alone) control run. The buffer may optionally be 0.01 M HEPES (4-(2-hydroxyethyl)-1-piperazineethanesulfonic acid), 0.15 M NaCl and 0.05% v/v surfactant P20 in aqueous solution, buffered to pH 7.4; and
4. Determining the affinity of binding of test antibody to test antigen using surface plasmon resonance. KD, Ka and Kd may then be calculated.
   SPR can be carried out using any standard SPR apparatus, such as by Biacore™ or using the ProteOn XPR36™ (Bio-Rad®).
   Regeneration of the capture surface can be carried out with 3 M magnesium chloride solution. This removes the captured test antibody and allows the surface to be used for another interaction. The binding data can be fitted to 1:1 model inherent using standard techniques, e.g. using analysis software such as Biacore Insight Evaluation Software.

### Cross-Reactivity:

In some examples, an antibody that specifically binds to a SARS-CoV-2 spike protein antigen does not cross-react with other antigens (but may optionally cross-react with SARS-CoV spike protein and/or MERS-CoV spike protein). In some examples, an antibody that specifically binds to a SARS-CoV-2 spike protein antigen does not cross react with the existing endemic seasonal coronaviruses (NL63, 229E, OC43 and HKU1).

In some examples, an antibody that specifically binds to a SARS-CoV-2 spike protein antigen cross-reacts with SARS-CoV spike protein. In some examples, an antibody that specifically binds to a SARS-CoV-2 spike protein antigen cross-reacts with MERS spike protein. In some examples, an antibody that specifically binds to a SARS-CoV-2 spike protein antigen cross-reacts with SARS-CoV spike protein and MERS spike protein.

For antibodies that specifically bind to a SARS-CoV-2 spike protein antigen and cross-react with SARS-CoV spike protein and/or MERS spike protein, in some examples, the antibody may bind SARS-CoV-2 spike protein with at least a 10 fold greater binding affinity than to SARS-CoV spike protein and/or MERS spike protein (e.g. as measured by SPR). In some examples, the antibody may bind SARS-CoV-2 spike protein with at least a 20 fold greater binding affinity than to SARS-CoV spike protein and/or MERS spike protein (e.g. as measured by SPR). In some examples, the antibody may bind SARS-CoV-2 spike protein with at least a 50 fold greater binding affinity than to SARS-CoV spike protein and/or MERS spike protein (e.g. as measured by SPR).

### Function—Inhibition, Neutralisation, Etc:

Antibodies described herein are inhibitory antibodies that inhibit a function of the SARS-CoV-2 spike protein, thus being useful in therapy and prophylaxis to prevent infection. In an example, the antibodies inhibit or prevent SARS-CoV-2 entering cells. In an example, the antibodies are neutralising antibodies.

In some examples, the antibodies inhibit the SARS-CoV-2 spike protein binding to the human ACE2 receptor. Inhibition of SARS-CoV-2 spike protein binding to the human ACE2 receptor may be achieved by an antibody directly blocking the epitope on the SARS-CoV-2 spike protein which binds to the human ACE2 receptor. Such antibodies may compete for binding to SARS-CoV-2 spike protein with the human ACE2 receptor, as described further below. Alternatively, inhibition of SARS-CoV-2 spike protein binding to the human ACE2 receptor may be achieved by an indirect mechanism, e.g. where an antibody binds to an epitope of the SARS-CoV-2 spike protein outside of the epitope on the SARS-CoV-2 spike protein which binds to the human ACE2 receptor, but which modifies the structure or function of the spike protein such that binding to human ACE2 receptor is reduced or prevented or the process of infecting the cell after ACE2 receptor binding is inhibited.

Human ACE2 (angiotensin-converting enzyme 2) is encodable by the mRNA sequence deposited in GenBank under accession number AB193259.1. ACE2 having the amino acid sequence from this accession number may be used in assays herein. The expression vector pCAGGS-ACE2 which was used in Examples herein comprised this coding sequence.

Human TMPRSS2 (transmembrane serine protease 2), which cleaves the spike protein, is encodable by the mRNA sequence deposited under NCBI reference sequence NM_001135099.1. TMPRSS2 having the amino acid sequence from this accession number may be used in assays herein. The expression vector pCAGGS-TMPRSS2 which was used in Examples herein comprised this coding sequence.

An inhibitory or neutralising antibody may bind either ofthe subunits (S1 or S2) of the SARS-CoV-2 spike protein. An inhibitory or neutralising antibody may bind any of the domains of the S1 subunit (e.g. RTB or NTD or a non-RBD/NTD domain) of the SARS-CoV-2 spike protein. Thus, in some examples, an inhibitory antibody that specifically binds to the receptor binding domain (RBD) of the SARS-CoV-2 spike protein is provided. In some examples, a neutralising antibody that specifically binds to the receptor binding domain (RBD) of the SARS-CoV-2 spike protein is provided. In some examples, an antibody that specifically binds to the receptor binding domain (RBD) of the SARS-CoV-2 spike protein, wherein the antibody inhibits or prevents SARS-CoV-2 entering cells is provided. In other examples, an inhibitory antibody that specifically binds to the S2 subunit of the SARS-CoV-2 spike protein is provided. In some examples, a neutralising antibody that specifically binds to the S2 subunit of the SARS-CoV-2 spike protein is provided. In some examples, an antibody that specifically binds to the S2 subunit of the SARS-CoV-2 spike protein, wherein the antibody inhibits or prevents SARS-CoV-2 entering cells is provided.

### Neutralisation—Measurement:

The ability of an antibody to neutralise SARS-CoV-2 entry to cells may be determined by in vitro assays. A widely used assay is the pseudovirus neutralisation assay, which uses a non-replication-competent virus-like particle with the SARS-CoV-2 spike protein within the virus envelope. Pseudotyped virus neutralisation assays using replication-deficient viruses are commonly used as a replacement for the use of wild-type viruses when studying pathogenic human viruses, which would otherwise need to be handled at higher levels of containment. The neutralizing ability of an antibody can be measured in a pseudotype neutralization assay using spike SARS-CoV-2 enveloped lentiviral pseudotypes carrying a firefly luciferase reporter. The use of such a reporter results in a wide dynamic range of neutralization titers and a high level of sensitivity. When the lentiviral genome integrates after entry into cells, firefly luciferase expression and activity is proportional to the number of cells that were infected (transduced) by the pseudotyped virus.

The pseudotype neutralization assay described herein, and detailed in Example 4, is a cell-based viral neutralization assay that is performed in a 384-well format. For the SARS-CoV-2 pseudotype neutralization, LentiX 293T cells (ATCC, CRL-3216) are used which are cultured and maintained in DMEM with 10% FBS added. The cells are prepared on day one, 24h later the cells are transiently transfected to express ACE2 (the SARS-CoV-2 viral receptor) and TMPRSS2 (the protease required for viral entry). After 24 h the ACE2/TMPRSS2 transiently transfected target cells are ready for use.

Serial dilutions of antibodies are prepared in a 384-well format and incubated with an appropriate titre (50-100 TCID50) of lentiviral particles for one hour at 37° C. The starting concentration of antibodies ranges from 50 nm to 100 nM, upon which 3- to 5-fold 8-point dilutions are performed. Each assay includes the following controls: cells only, cells and pseudovirus, positive and negative control antibodies.

After one hour, ACE2/TMPRSS2 transiently transfected target cells are added to the wells and the plates are incubated for 48 h at 37° C. to permit cell infection (transduction) of non-neutralized particles and expression of firefly luciferase. Following the 48h incubation cells are lysed for 5 min in the presence of a luciferase substrate (e.g., Bright-Glo Luciferase Assay System (Promega)) to assess luciferase activity. After this 5 min incubation at room temperature the luminescence in each well is measured.

The comparison between the luciferase signal detected in uninfected (untransduced) cells, in cells infected (transduced) with pseudotypes only, and in cells infected (transduced) with pseudotypes in the presence of antibodies, enables us to determine if the antibody has neutralizing activity against the SARS-CoV-2 pseudotype tested.

Alternatively, the ability of an antibody to neutralise wild type, replication competent, authentic SARS-CoV-2 entry to cells may be determined by in vitro assays which are known as live virus assays. The live virus assay involves producing a laboratory stock of SARS-CoV-2 virus from an isolate ofthe virus derived from an infected person. Each isolate from different people results in a different live virus stock, which will normally have the full virus genome sequence determined to ensure the virus is not defective in any gene and to determine where, if anywhere the virus isolate differs genetically from the wild type virus genome sequence and if the genetic changes alter the amino acid sequence of a virus protein. Live virus isolates can be produced by culturing the SARS-CoV-2 virus on human cells or on animal cells in vitro, providing the cells are permissive to virus replication. Two known factors that confer permissivity are the ACE-2 receptor and the cell surface protein TMPRSS2. Some primary human cells naturally express these proteins, such as primary human airway epithelial cells (PAE cells), some cancer cell lines naturally express these proteins, such as Caco-2 and Calu-3, some human cells can be made to express these proteins artificially such as 293T cells transiently transfected to express ACE2 (the SARS-CoV-2 viral receptor) and TMPRSS2 and some animal cells are naturally permissive to SARS-CoV-2 such as Vero E6 cells from the African Green Monkey. Therefore, all live virus assays are related but often with specific differences. The neutralizing ability of an antibody can be measured in a live virus neutralization assay by incubating a known fixed amount of the live virus with different dilutions of the antibody, and then following incubation, adding the mixture to cells that are permissive for SARS-CoV-2 infection. The cells are then incubated to allow virus infection and replication to occur. Detection of virus infection and replication can be determined by a number of methods, including, colourimetry detection and quantitation of infected cells using labelled antibodies to a SAR-CoV-2 protein such as the Nucleoprotein (N), or visualisation of infected cell foci by staining and enumeration of cells stained with a labelled antibody to a SAR-CoV-2 protein such as the Nucleoprotein (N). The amount of reduction in cell infection caused by an antibody is calculated relative to a control infection where no antibody is added. These assays can be performed in 24, 48 or 96 well tissue culture plates.

The neutralising ability of an antibody of the invention can be determined in vitro according to the methods for pseudovirus neutralisation and/or live virus neutralisation. In both cases the concentration of the antibody, expressed as either or both of the antibody weight (milligrams, or micrograms, or nanograms or picograms) in a given volume (litre or millilitre or microlitre), or as a molarity of the antibody (millimolar, or micromolar or nanomolar or picomolar), that is required to inhibit 50% of the detectable infection in the assay (the inhibitory concentration for 50%, or IC50) or inhibit 90% of the detectable infection in the assay (the inhibitory concentration for 90%, or IC90) or inhibit 95% of the detectable infection in the assay (the inhibitory concentration for 95%, or IC95) is reported. This can be calculated using any of a variety of methods known to the art, including the fitting of inhibition curves mathematically to the experimentally derived data and reporting these as an IC50 or IC90 or IC95. Finally, the antibody concentration that completely inhibits SARS-CoV-2 infection of cells can be determined. This value will be similar to the IC95 value and this was determined for the data in Table E5-1.

An example protocol for the pseudovirus neutralisation assay is provided in Example 4 herein.

In some examples, the antibodies neutralise SARS-CoV-2 with an IC50 of 10 nM or lower (e.g. as determined in a pseudovirus assay). In some examples, the antibodies neutralise SARS-CoV-2 with an IC50 of lnM or lower (e.g. as determined in a pseudovirus assay). In some examples, the antibodies neutralise SARS-CoV-2 with an IC50 of 500 pM or lower (e.g. as determined in a pseudovirus assay). In some examples, the antibodies neutralise SARS-CoV-2 with an IC50 of 100 pM or lower (e.g. as determined in a pseudovirus assay). In some examples, the antibodies neutralise SARS-CoV-2 with an IC50 of 50 pM or lower (e.g. as determined in a pseudovirus assay). In some examples, the antibodies neutralise SARS-CoV-2 with an IC50 of 40 pM or lower (e.g. as determined in a pseudovirus assay). In some examples, the antibodies neutralise SARS-CoV-2 with an IC50 of 30 pM or lower (e.g. as determined in a pseudovirus assay). In some examples, the antibodies neutralise SARS-CoV-2 with an IC50 of 20 pM or lower (e.g. as determined in a pseudovirus assay). In some examples, the antibodies neutralise SARS-CoV-2 with an IC50 of 10 pM or lower (e.g. as determined in a pseudovirus assay). In some examples, the antibodies neutralise SARS-CoV-2 with an IC50 of 5 pM or lower (e.g. as determined in a pseudovirus assay).

In some examples, the antibodies neutralise SARS-CoV-2 with an IC50 of 10 nM or lower (e.g. as determined in a live virus assay). In some examples, the antibodies neutralise SARS-CoV-2 with an IC50 of lnM or lower (e.g. as determined in a live virus assay). In some examples, the antibodies neutralise SARS-CoV-2 with an IC50 of 500 pM or lower (e.g. as determined in a live virus assay). In some examples, the antibodies neutralise SARS-CoV-2 with an IC50 of 100 pM or lower (e.g. as determined in a live virus assay). In some examples, the antibodies neutralise SARS-CoV-2 with an IC50 of 50 pM or lower (e.g. as determined in a live virus assay). In some examples, the antibodies neutralise SARS-CoV-2 with an IC50 of 40 pM or lower (e.g. as determined in a live virus assay). In some examples, the antibodies neutralise SARS-CoV-2 with an IC50 of 30 pM or lower (e.g. as determined in a live virus assay).

In some examples, the antibodies may neutralise SARS-CoV-2 with an activity level which is greater than a reference antibody. In some examples, the reference antibody may be SAD S35 (Acro Biosystems; https://www.acrobiosystems.com/P3209-Anti-SARS-CoV-2-RBD-Neutralizing-Antibody-Human-IgG1.html). In some examples, the reference antibody may be 4A8 (Chi et al., Science vol. 369 (6504), 650-655). For example, the antibodies may neutralise SARS-CoV-2 with an activity level which is greater than the reference antibody expressed as fold change relative to the reference antibody. In one example, the antibody may neutralise SARS-CoV-2 with an activity level greater than a 2-fold change relative to the reference antibody. In one example, the antibody may neutralise SARS-CoV-2 with an activity level greater than a 25-fold change relative to the reference antibody. In one example, the antibody may neutralise SARS-CoV-2 with an activity level greater than a 50-fold change relative to the reference antibody. In one example, the antibody may neutralise SARS-CoV-2 with an activity level greater than a 100-fold change relative to the reference antibody. In one example, the antibody may neutralise SARS-CoV-2 with an activity level greater than a 500-fold change relative to the reference antibody. In one example, the antibody may neutralise SARS-CoV-2 with an activity level greater than a 1000-fold change relative to the reference antibody.

The antibodies provided may inhibit the SARS-CoV-2 spike protein binding to the human ACE2 receptor.

In some examples, an antibody specifically binds to the receptor binding domain (RBD) of the SARS-CoV-2 spike protein, wherein the antibody inhibits the SARS-CoV-2 spike protein binding to the human ACE2 receptor. Various modes of inhibition may be envisaged. For instance, inhibition may be by competition for binding to ACE2 (whether for the same epitope or by steric hindrance), or inhibition may be through the prevention of RBD becoming in its UP states leading to inhibiting ACE2 interaction with RBD.

In some examples, an antibody specifically binds to the S2 subunit of the SARS-CoV-2 spike protein, wherein the antibody inhibits a function of the SARS-CoV-2 spike protein binding to the human ACE2 receptor triggering entry into the cell. Again, various modes of inhibition may be envisaged. For example, an anti-S2 antibody may inhibit fusion with the host cell membrane or it may inhibit the cleavage of S1 and S2 by TMPRSS2 or cathepsins or other cellular protease that can modify the spike protein.

Competition with Ace2:

The antibodies provided may compete with the SARS-CoV-2 spike protein for binding to the human ACE2 receptor. The antibodies provided may specifically bind to the receptor binding domain (RBD) of the SARS-CoV-2 spike protein, wherein the antibody is a neutralising antibody which competes with the SARS-CoV-2 spike protein for binding to the human ACE2 receptor, thereby preventing cell infection. Other antibodies, that do not compete with the SARS-CoV-2 spike protein for binding to the human ACE2 receptor, may alter the ability of the spike protein to function correctly and thereby also be a neutralising antibody even though the antibody does not block RBD and ACE2 interaction.

Whether an antibody competes with the SARS-CoV-2 spike protein for binding to the human ACE2 receptor may be measured using a competition assay. Competition may be determined by surface plasmon resonance (SPR), such techniques being readily apparent to the skilled person. SPR may be carried out using Biacore™, Proteon™ or another standard SPR technique. Such competition may be due, for example, to the antibodies or fragments binding to identical or overlapping epitopes of the SARS-CoV-2 spike protein to that which the ACE2 receptor binds. In one example, competition is determined by ELISA, such techniques being readily apparent to the skilled person. In one example, competition is determined by homogenous time resolved fluorescence (HTRF), such techniques being readily apparent to the skilled person. In one example, competition is determined by fluorescence activated cell sorting (FACS), such techniques being readily apparent to the skilled person. In one example, competition is determined by ForteBio Octet® Bio-Layer Interferometry (BLI) such techniques being readily apparent to the skilled person.

In one example, the antibody competes (e.g. in a dose-dependent manner) with SARS-CoV-2 spike protein (or a fusion protein thereof) for binding to cell surface-expressed human ACE2 receptor. In one embodiment, the antibody competes (e.g. in a dose-dependent manner) with SARS-CoV-2 spike protein (or a fusion protein thereof) for binding to soluble human ACE2 receptor.

In one example, the antibody partially or completely inhibits binding of SARS-CoV-2 spike protein to cell surface-expressed human ACE2 receptor. In another example, the antibody partially or completely inhibits binding of SARS-CoV-2 to soluble human ACE2 receptor.

If the epitope to which the antagonist antibody binds completely blocks the binding site of the ACE2 receptor, then receptor binding is completely prevented (which may be a physical blocking—in the case of overlapping epitopes—or steric blocking—where the antagonist is large such that it prevents the receptor binding to its distinct epitope). If the epitope to which the antibody binds partially blocks the binding site of the ACE2 receptor, the receptor may be able to bind, but only weakly (in the case of partial inhibition), or in a different orientation to the natural binding interaction.

### Other Modes—Destabilising:

In some examples, the antibody may destabilise the SARS-CoV-2 spike protein. Such antibodies may therefore disrupt binding of the SARS-CoV-2 spike protein to the human ACE2 receptor as a result of destabilising the spike protein thereby resulting in a beneficial therapeutic effect, either when the antibody is used alone or in combination with a further anti-SARS-CoV-2 antibody.

### Other Modes—Increased Binding:

In some examples, the antibody may increase the number of RBDs in the UP position with an apparent increase in the binding between the SARS-CoV-2 spike protein and the human ACE2 receptor in biochemistry assays, but with an overall inhibition of appropriate spike protein function in the virus particle, leading to neutralisation of the virus. Such antibodies may be particularly useful therapeutically when used in combination with another RBD binding and ACE-2 blocking anti-SARS-CoV-2 antibody. For example, they may destabilise the interactions within the SARS-CoV-2 spike protein trimer or may force the RBD of the SARS-CoV-2 spike protein into upward configuration more often which may make it more susceptible to a neutralising antibody that specifically binds the RBD of the SARS-CoV-2 spike protein. Data supporting this mode of action are presented in Example 3.

Such antibodies may also be particularly useful as diagnostic antibodies, especially if used in a double antigen binding assays where the antibody is used to capture the spike protein.

Exemplary antibodies described herein are set out in Tables 1a and 1b and described further below.

### Group a ‘Ace2-Competing’ RBD Binders:

In some aspects, the antibody specifically binds the RBD of the SARS-CoV-2 spike protein, wherein the antibody competes for binding to the SARS-CoV-2 spike protein with the human ACE2 receptor.

Provided herein are antibodies that neutralise SARS-CoV-2 and specifically bind to the receptor binding domain (RBD) of the S1 subunit of SARS-CoV-2 and compete with ACE2 for binding to SARS-CoV-2. In one example, the antibodies have a high affinity (such as a KD of 10-9 M or lower or even a KD of 5×10-10 M or lower) for the isolated RBD of the SARS-CoV-2 spike protein, particularly when measured using a surface plasmon resonance (SPR) assay (e.g. a Biacore SPR assay). In one example, the antibodies neutralise SARS-CoV-2 with high potency (such as with an IC50 of lnM or lower, an IC50 of 100 pM or lower, an IC50 of 50 pM or lower, an IC50 of 10 pM or lower, or even an IC50 of 5 pM or lower) particularly in vitro in pseudovirus assays. In one example, the antibodies neutralise SARS-CoV-2 with high potency (such as with an IC50 of lnM or lower, an IC50 of 100 pM or lower, an IC50 of 50 pM or lower, an IC50 of 30 pM) particularly in vitro in live virus assays. In one example, the antibodies (i) have a high affinity (such as a KD of 10-9 M or lower or even a KD of 5×10-10 M or lower) for the RBD of SARS-CoV-2 and (ii) neutralise SARS-CoV-2 with high potency (such as with an IC50 of lnM or lower, an IC50 of 100 pM or lower, an IC50 of 50 pM or lower, an IC50 of 10 pM or lower, or even an IC50 of 5 pM or lower).

In one example, the antibody is selected from the group consisting of IMPI-029, IMPI-056, IMPI-005, IMPI-012, IMPI-052, IMPI-002, IMPI-041, IMPI-036, IMPI-055, IMPI-054, IMPI-042, IMPI-021, IMPI-060, IMPI-006, IMPI-004, IMPI-047, IMPI-037, IMPI-017 and IMPI-059.

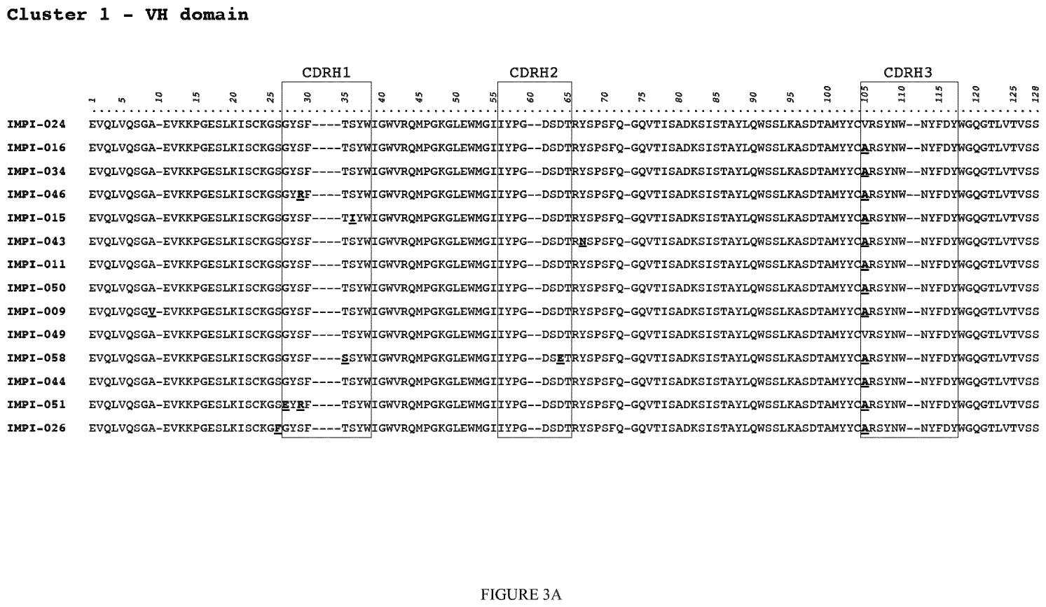

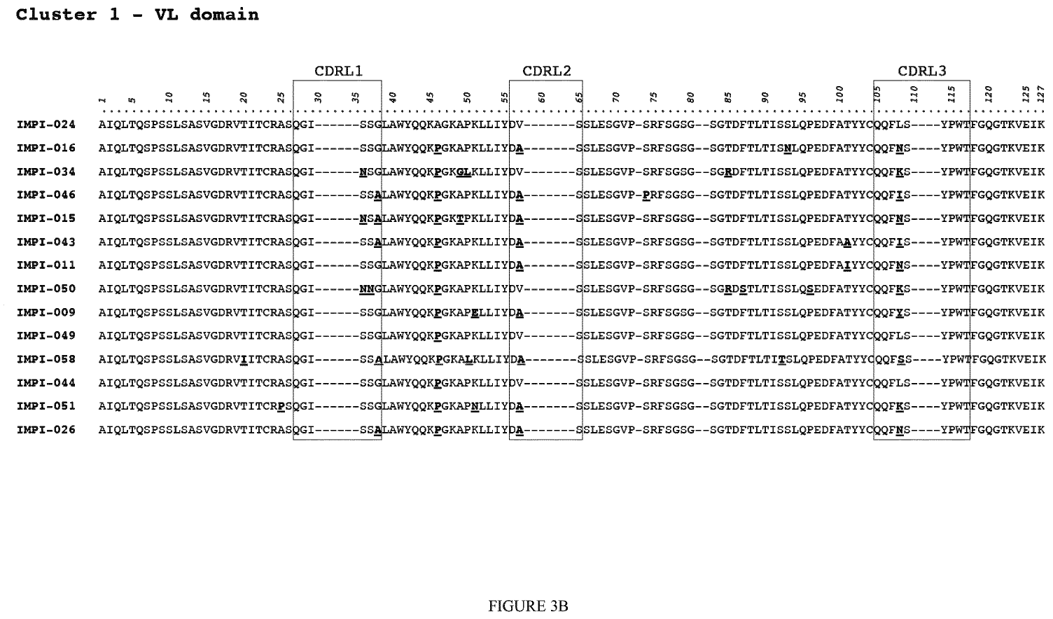

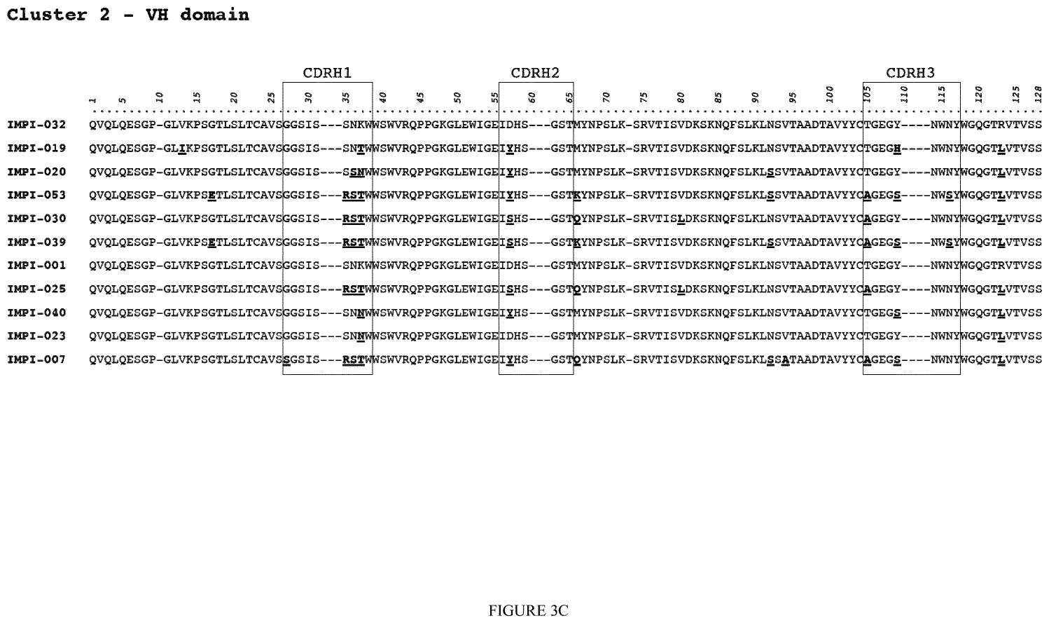

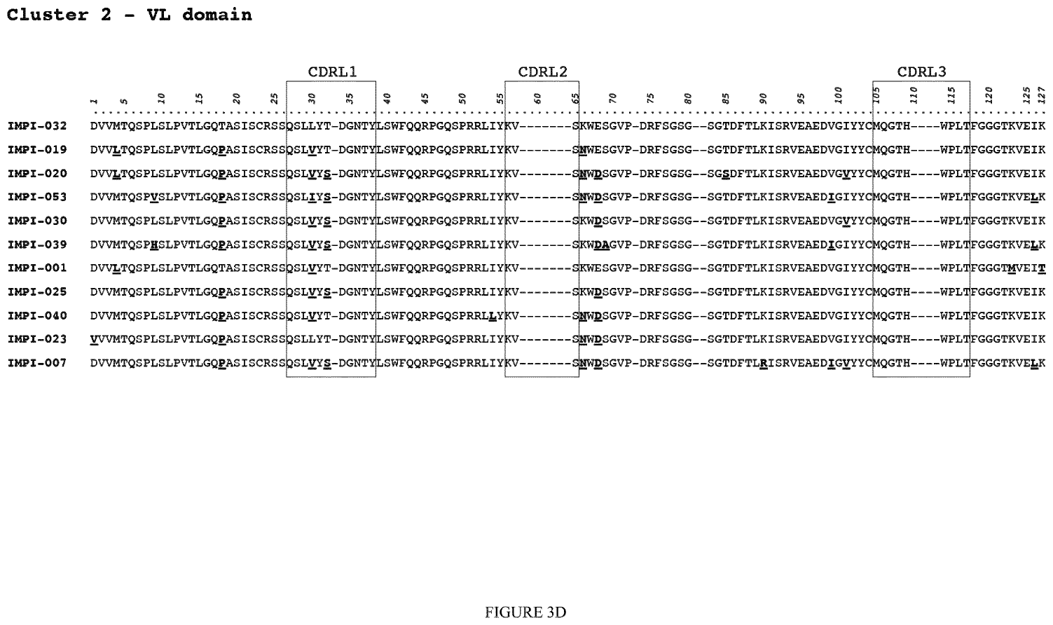

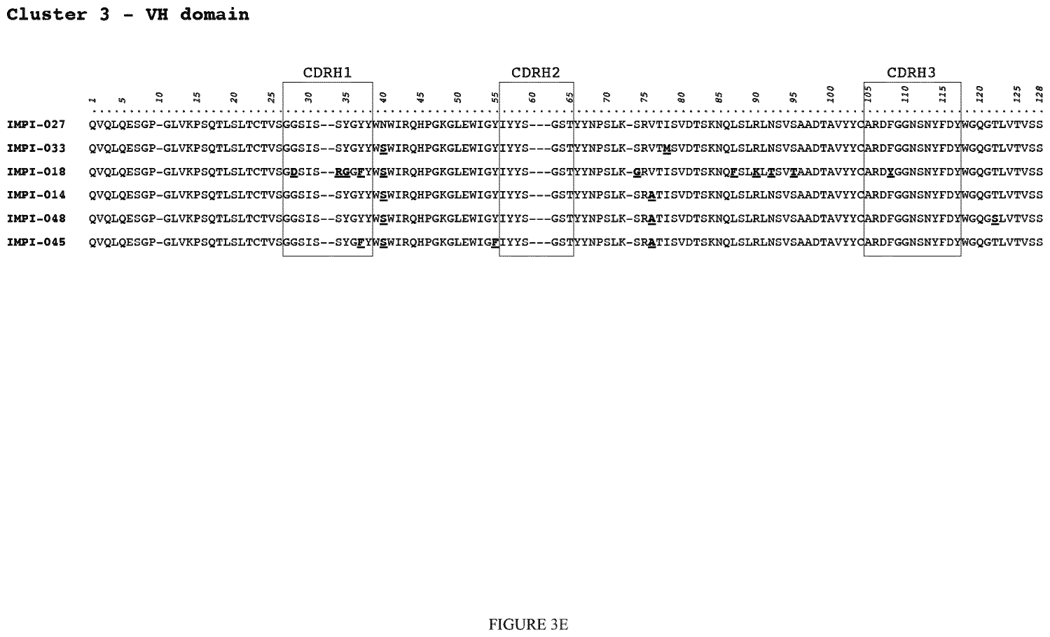

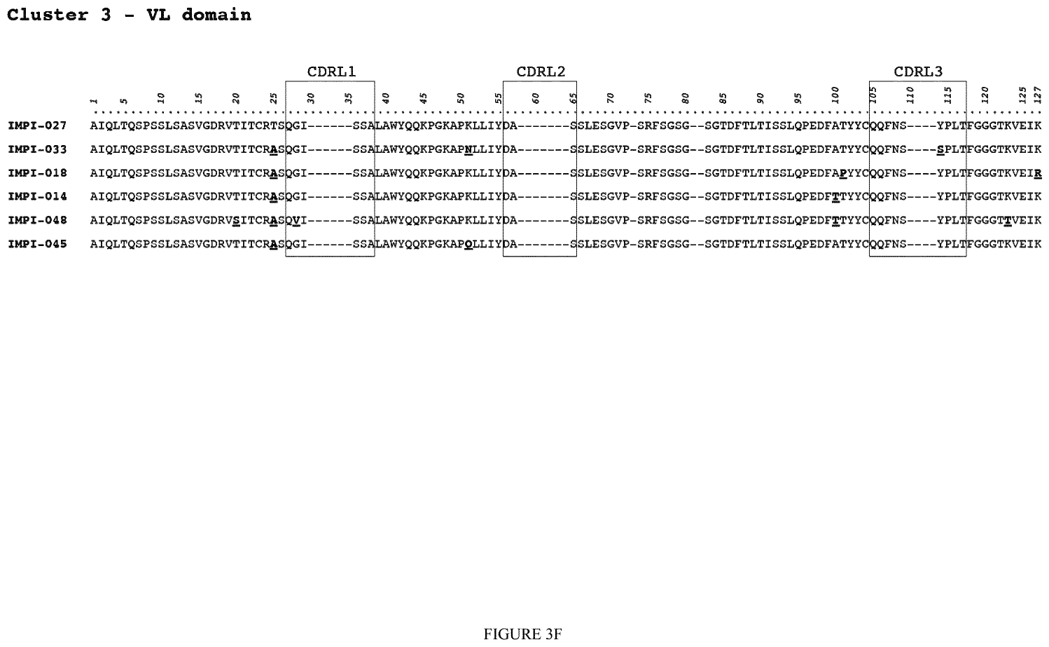

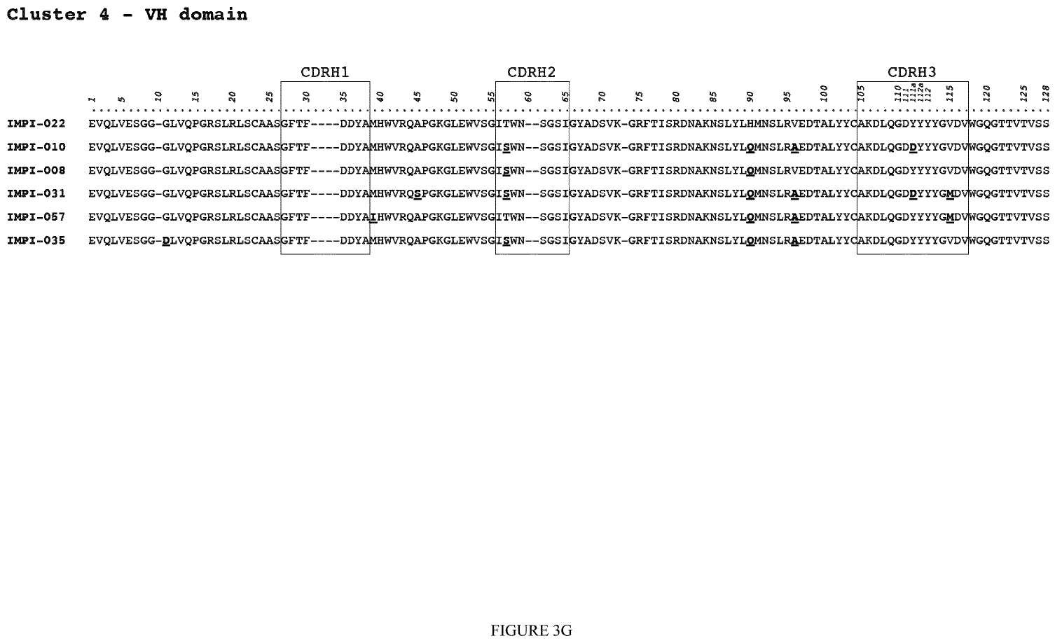

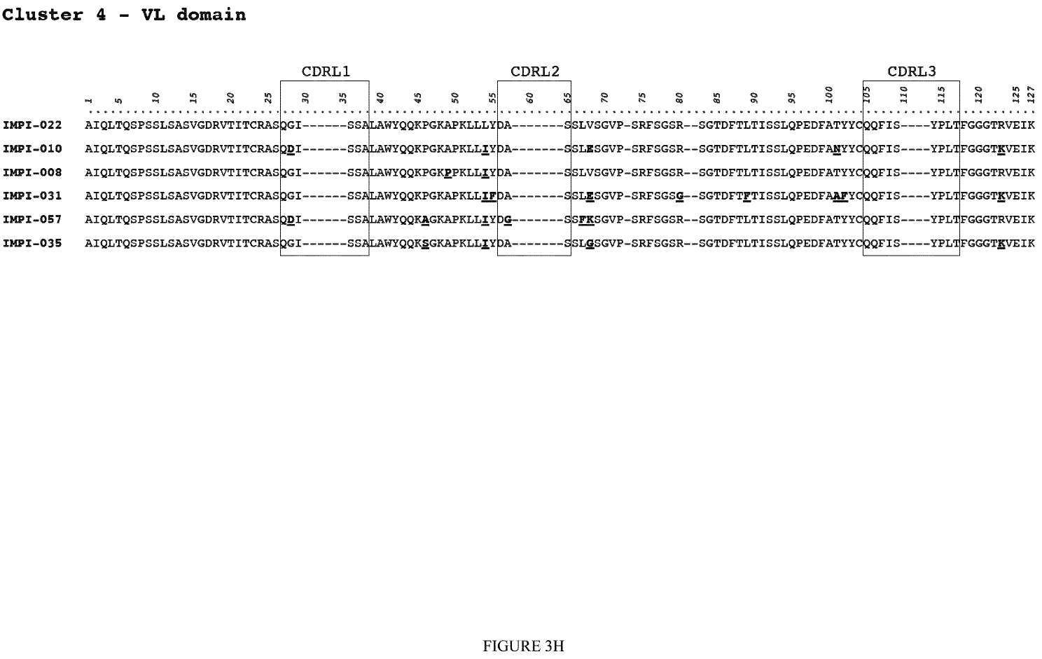

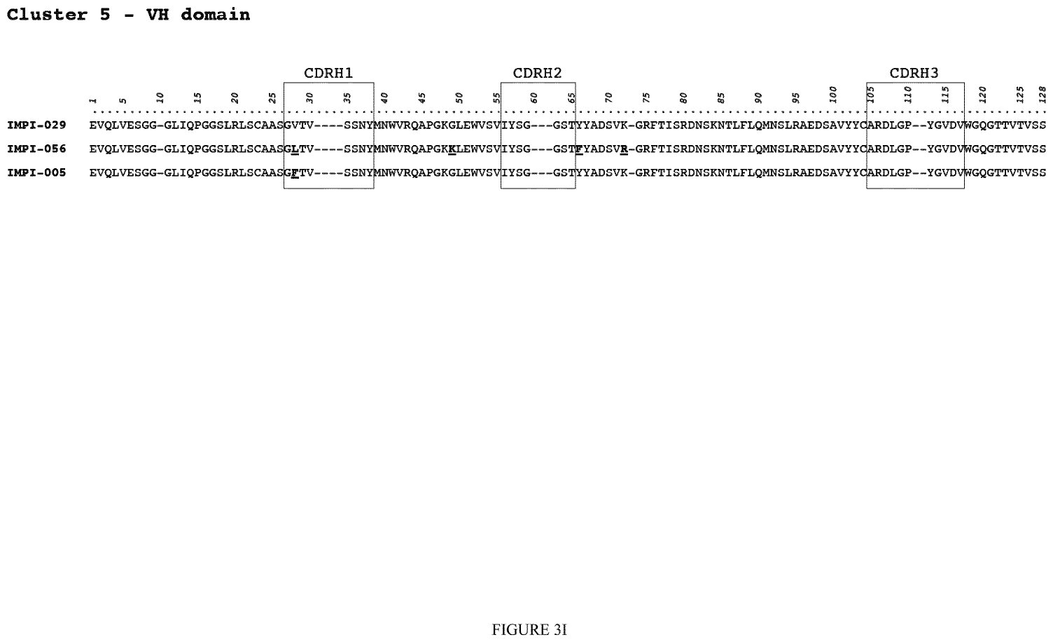

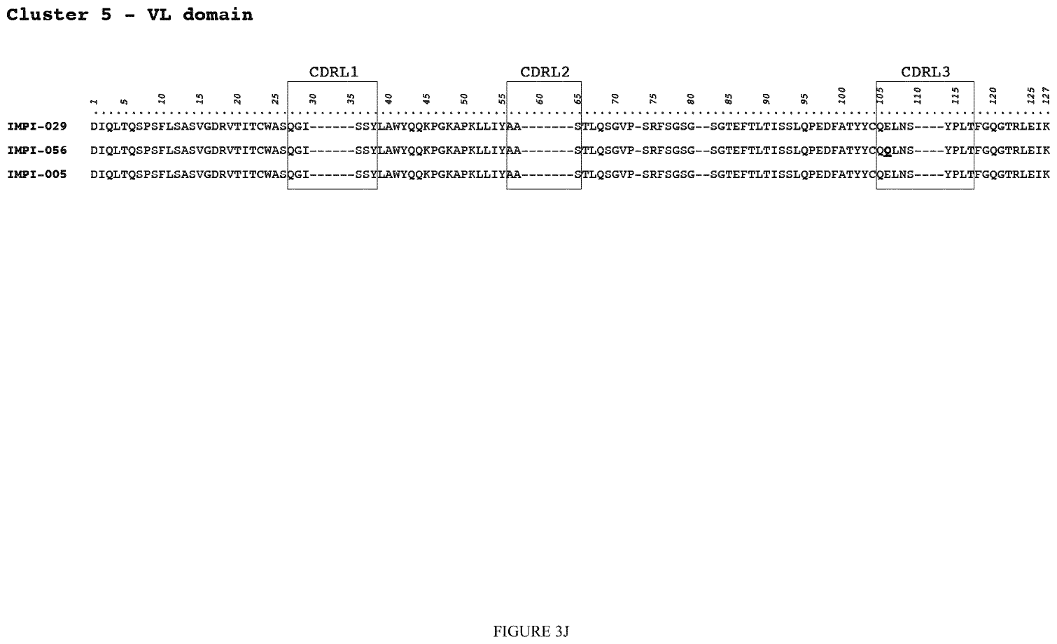

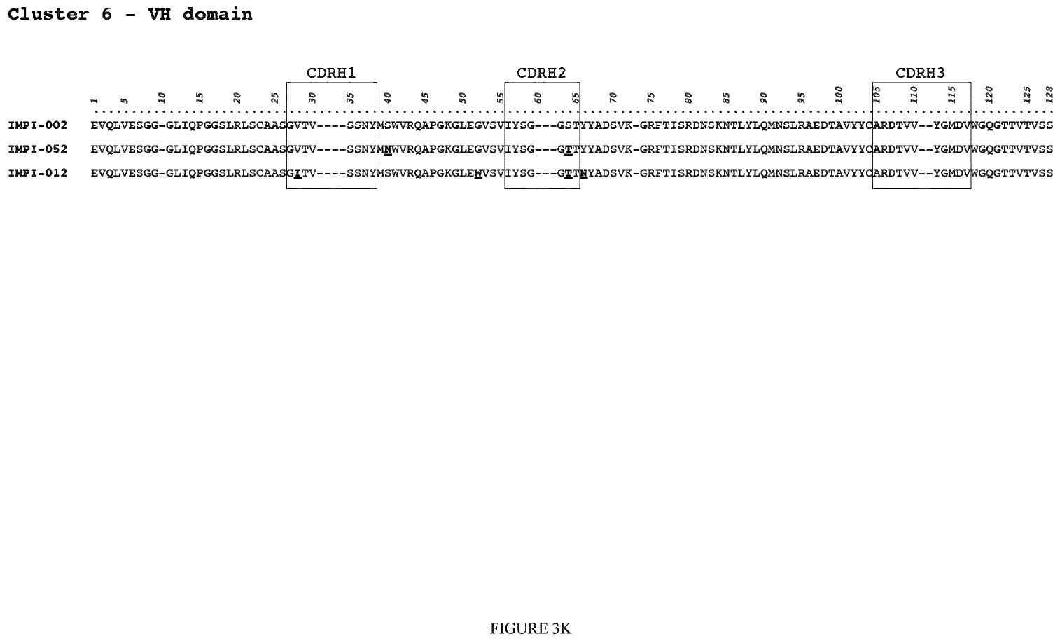

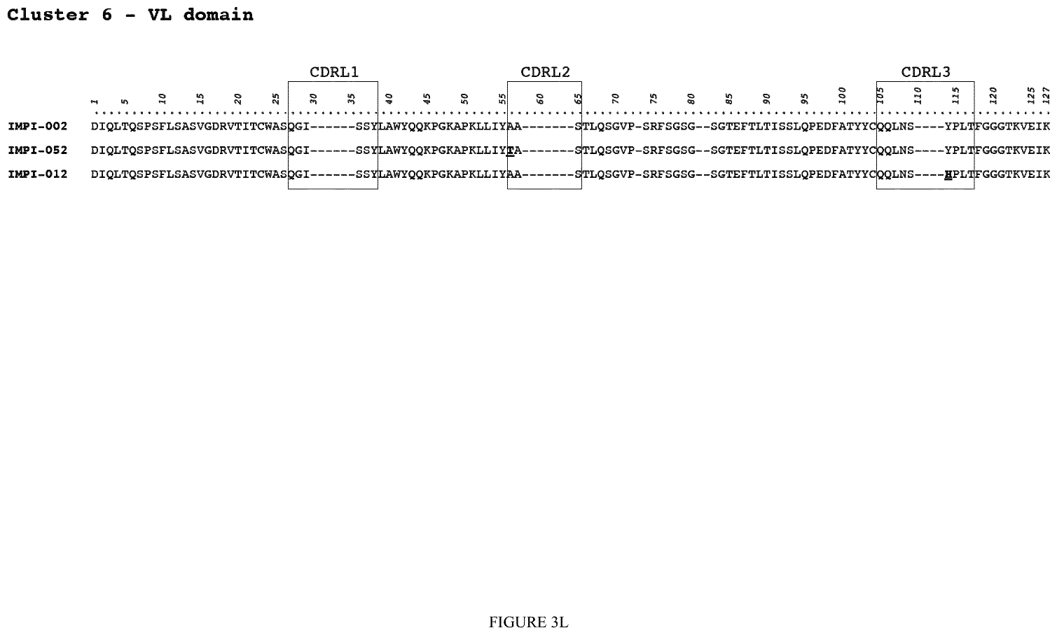

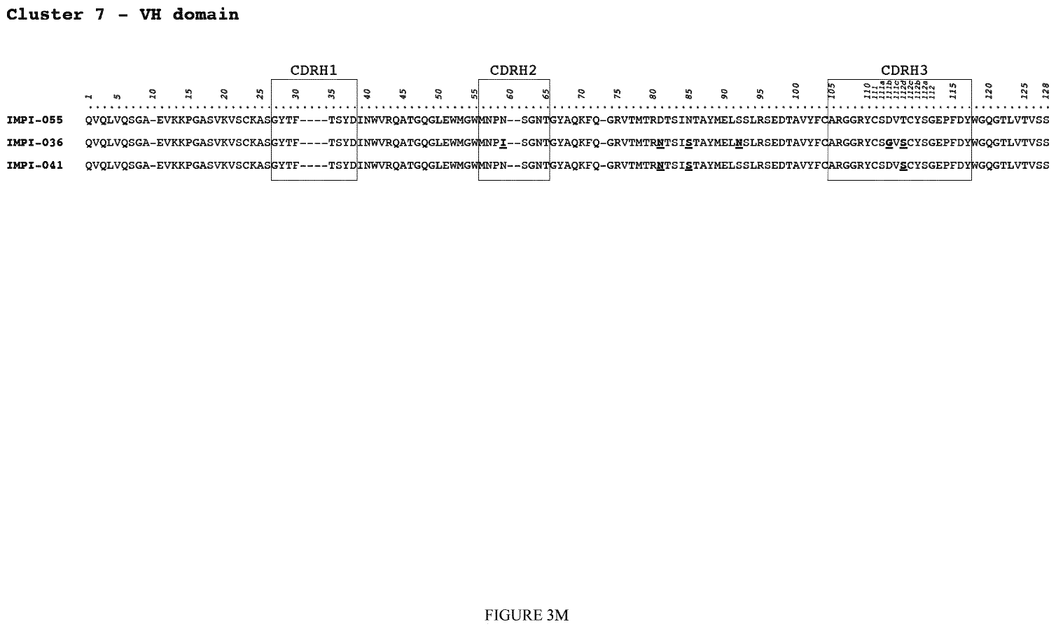

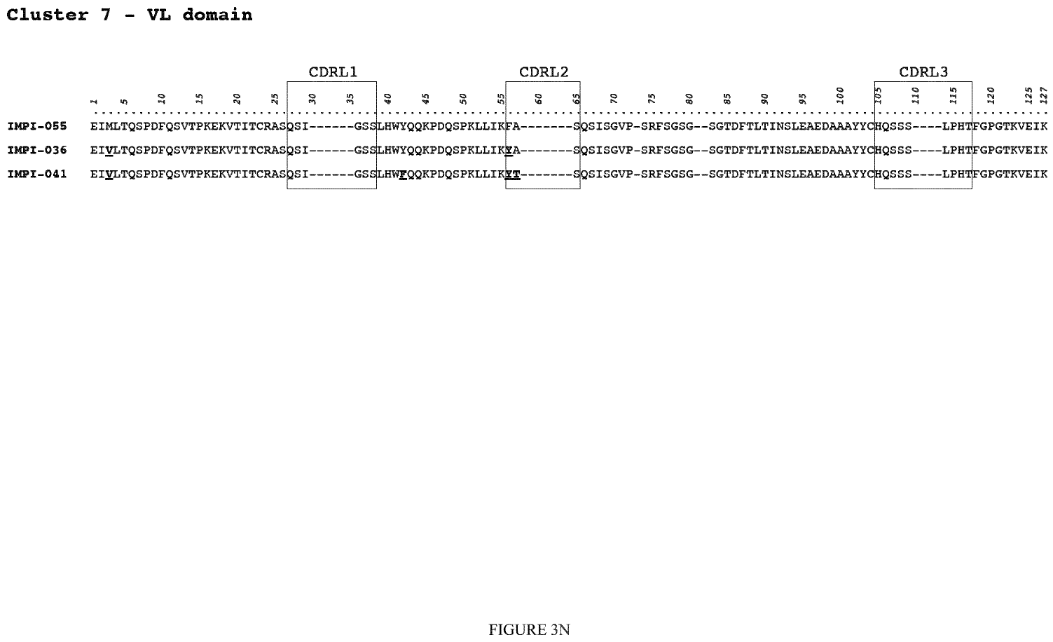

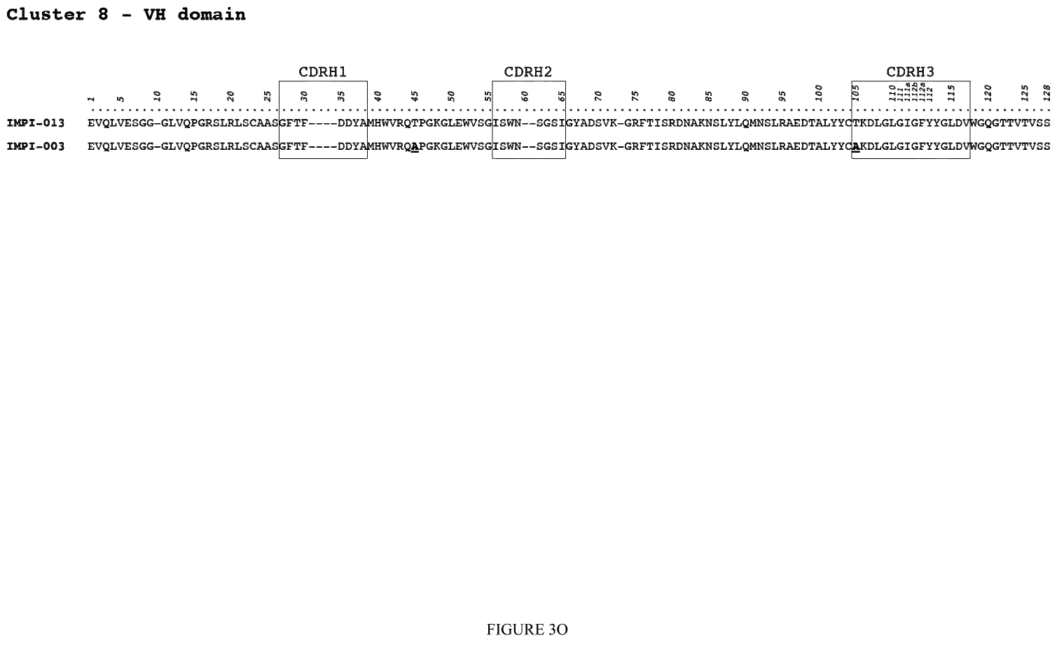

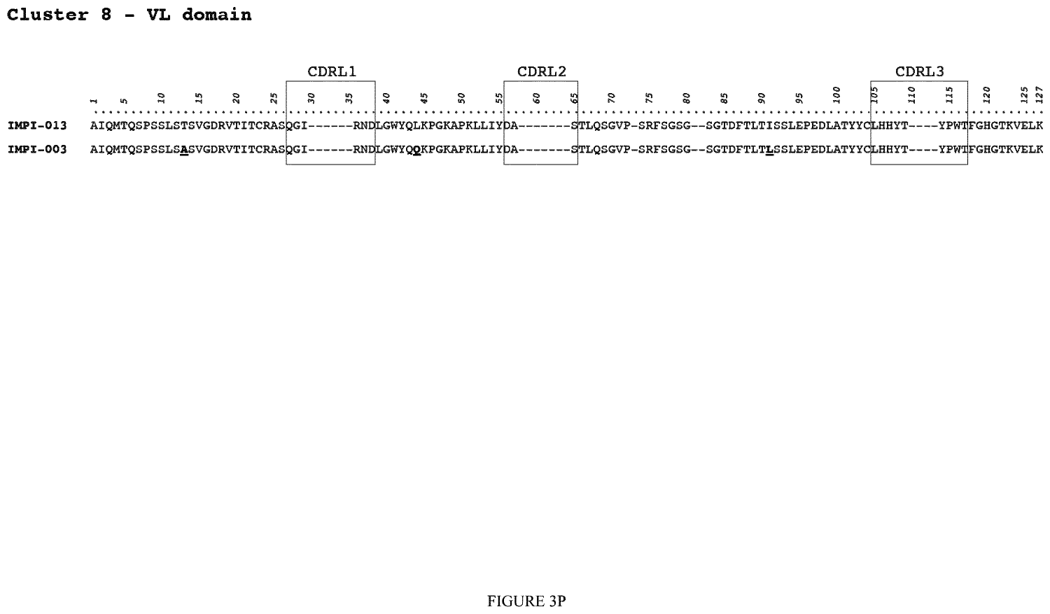

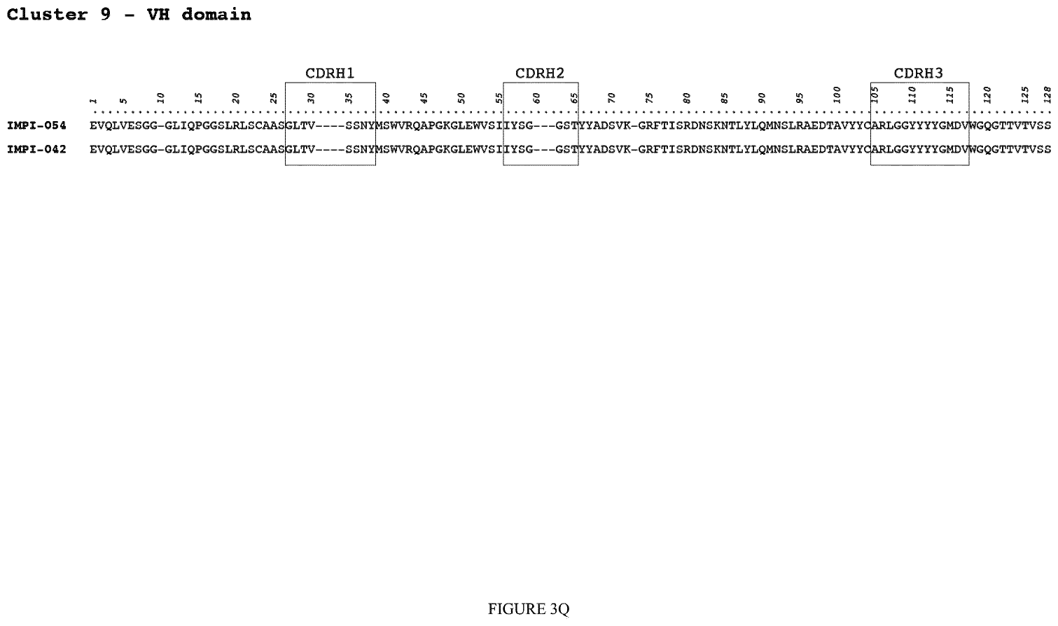

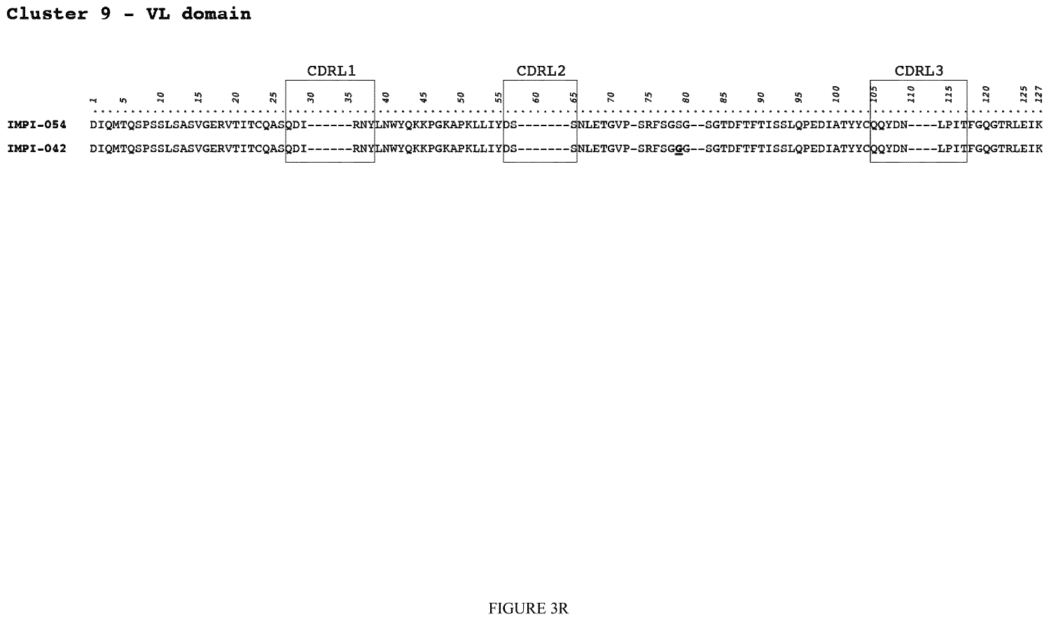

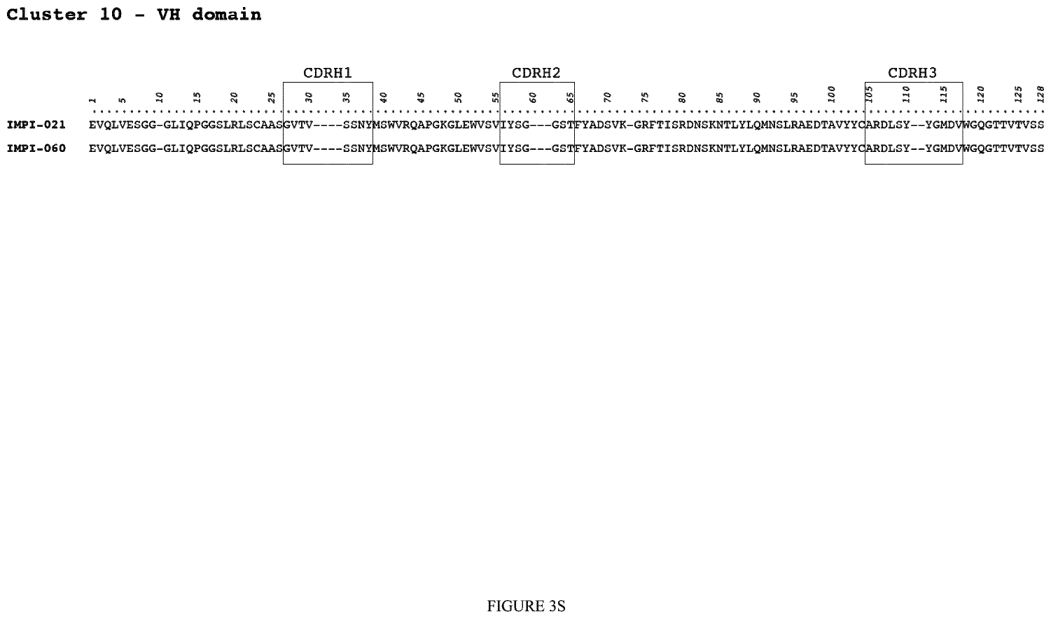

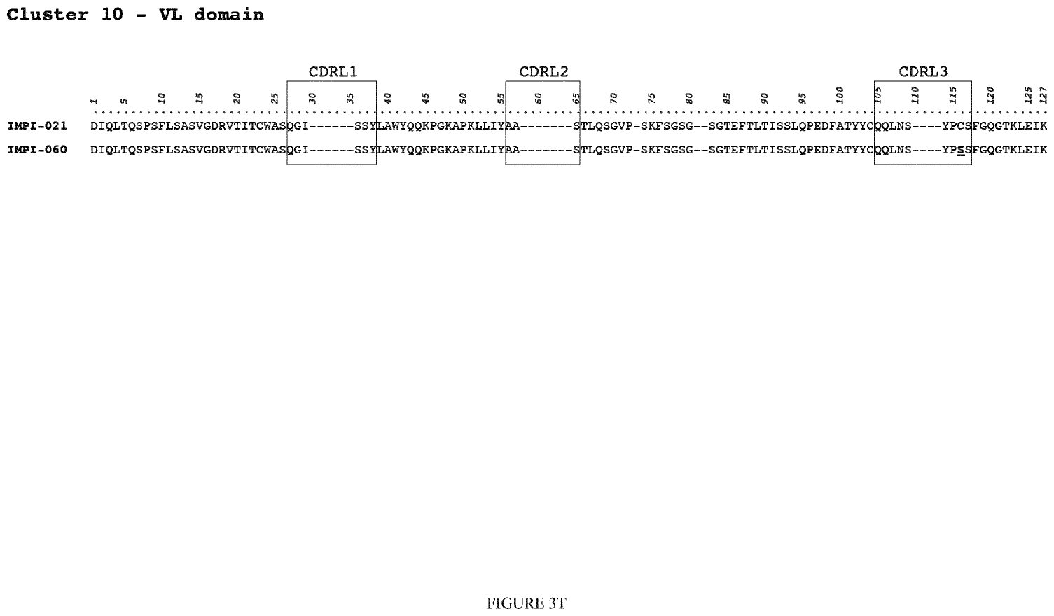

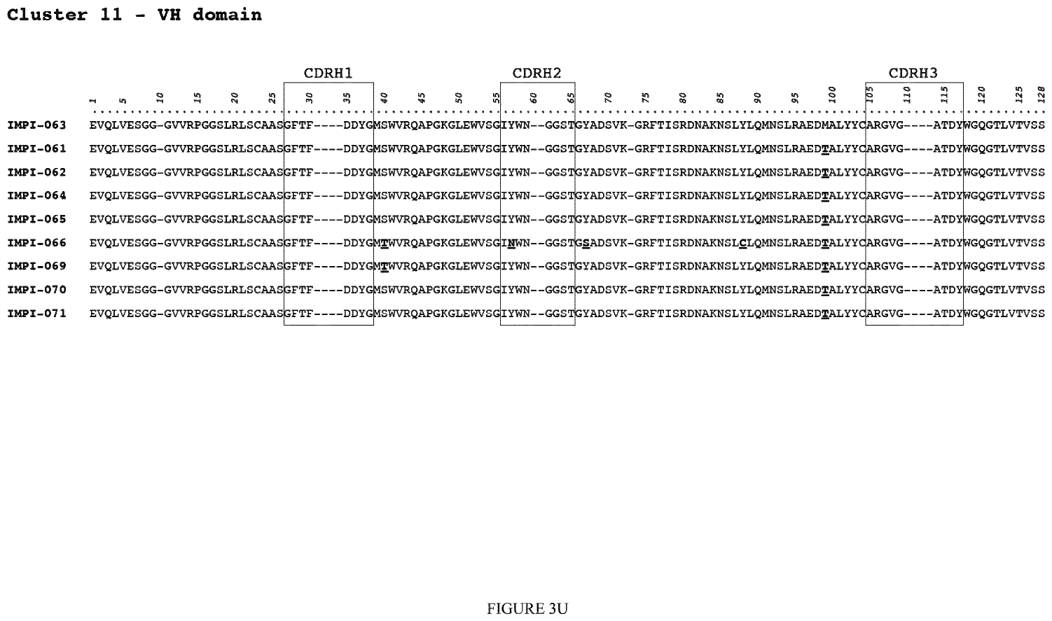

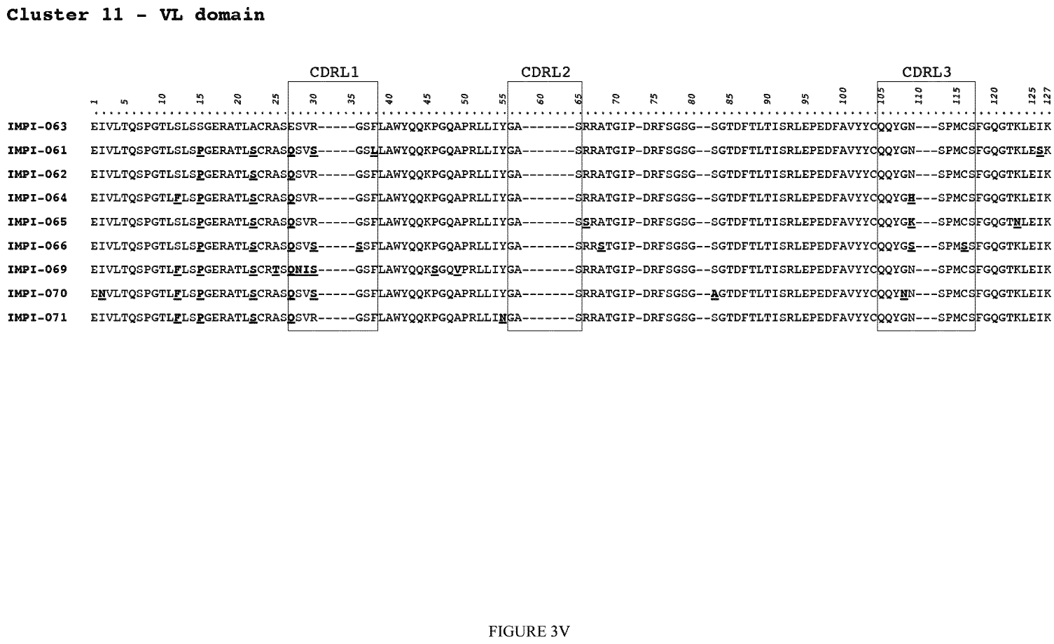

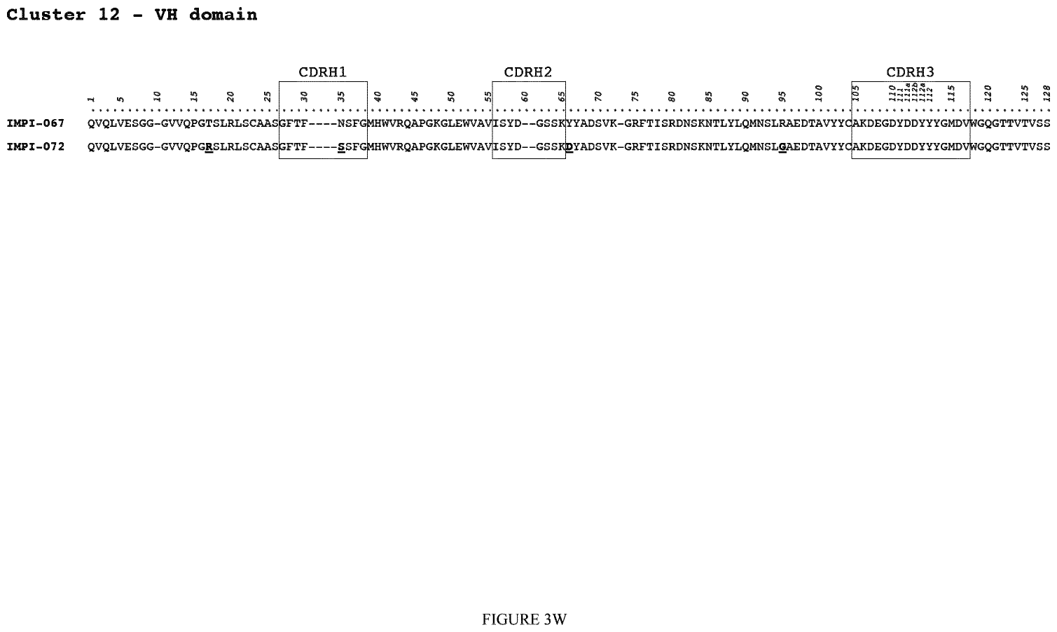

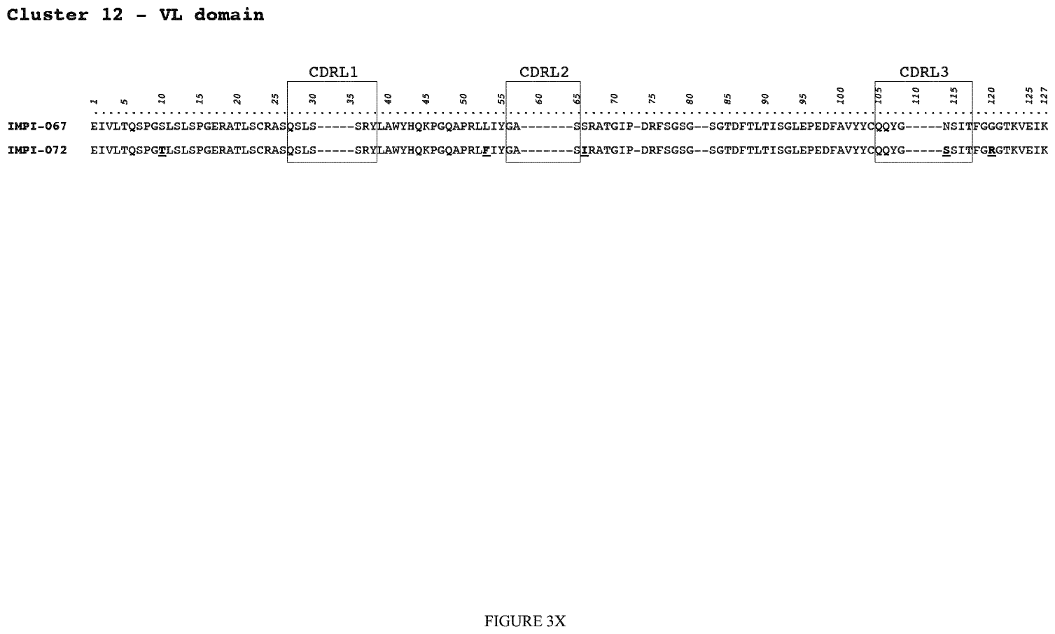

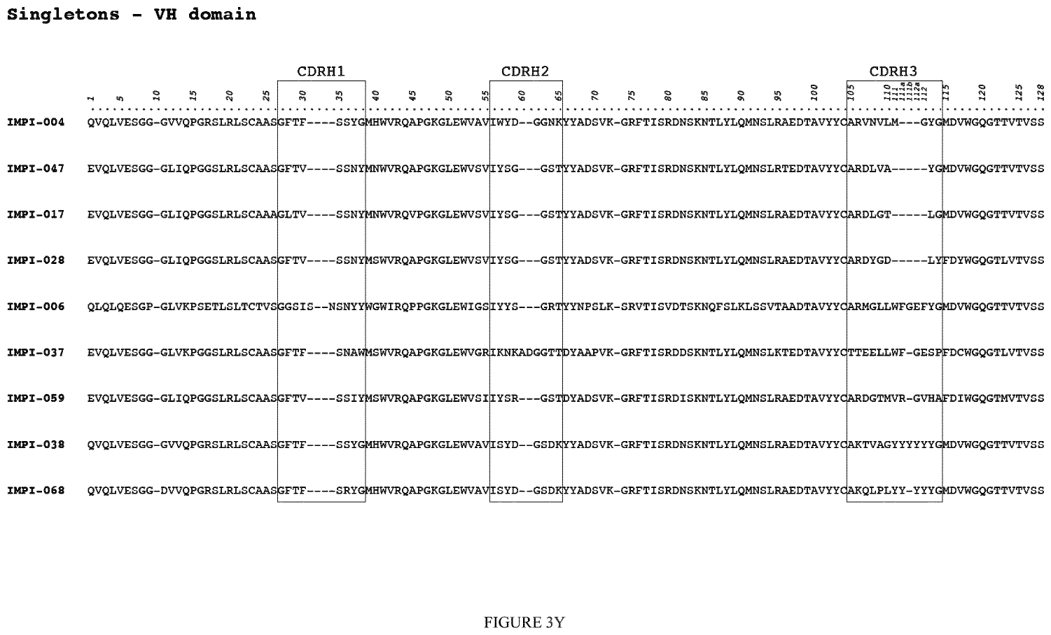

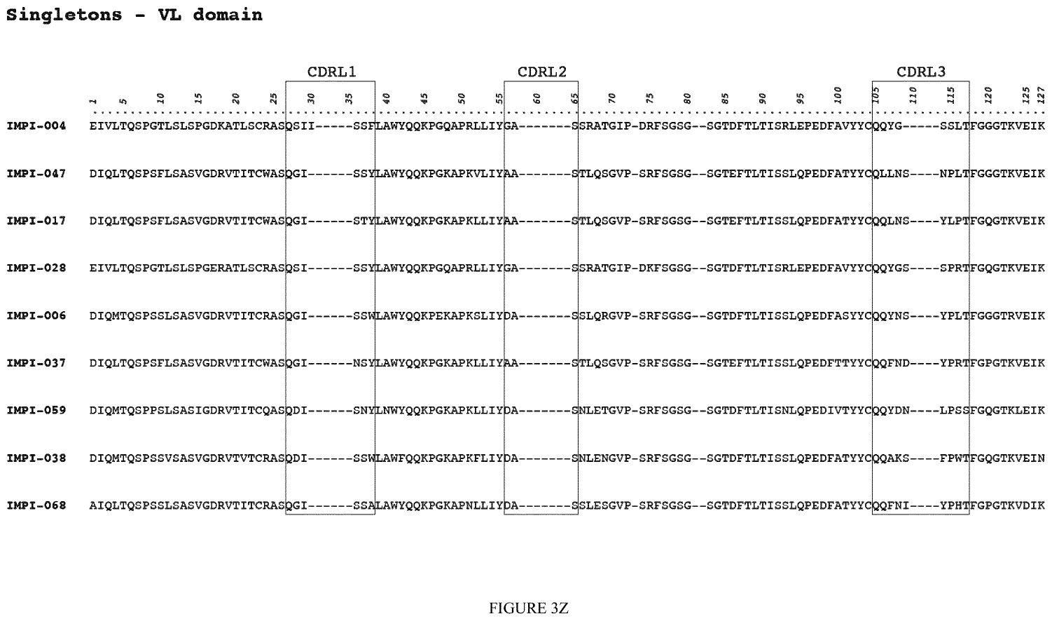

In one example, the antibody is selected from the group consisting of IMPI-029, IMPI-056 and IMPI-005 (cluster 5 in FIG. 3). In one example, the antibody is selected from the group consisting of IMPI-012, IMPI-052 and IMPI-002 (cluster 6 in FIG. 3). In one example, the antibody is selected from the group consisting of IMPI-041, IMPI-036 and IMPI-055 (cluster 7 in FIG. 3). In one example, the antibody is selected from the group consisting of IMPI-054 and IMPI-042 (cluster 9 in FIG. 3). In one example, the antibody is selected from the group consisting of IMPI-021 and IMPI-060 (cluster 10 in FIG. 3).

In one example, the antibody is IMPI-029, IMPI-056 or IMPI-005. In one example, the antibody is IMPI-012, IMPI-052 or IMPI-002. In one example, the antibody is IMPI-041, IMPI-036 or IMPI-055. In one example, the antibody is IMPI-054 or IMPI-042. In one example, the antibody is IMPI-021 or IMPI-060.

In one example, the antibody is selected from the group consisting of YANG-1101, YANG-1103, YANG-1105, YANG-1106, YANG-1107, YANG-1108, YANG-1109, YANG-1110, YANG-1112, YANG-1112a, YANG-1112b, YANG-1112c, YANG-1113, YANG-1114, YANG-1115, YANG-1116, YANG-1117, YANG-1118, YANG-1119, YANG-2101, YANG-2102, YANG-2103, YANG-2104, YANG-2105, YANG-2106, YANG-2107, YANG-2108, YANG-2108a, YANG-2108b, YANG-2108c, YANG-2108d, YANG-2108e, YANG-2108f, YANG-2108g, YANG-2108h, YANG-2108i, YANG-2108j, YANG-2108k, YANG-21081, YANG-2109, YANG-2110, or YANG-2111.

In one example, the antibody is selected from the group consisting of YANG-1112, YANG-2107, YANG-2108, or YANG-2111.

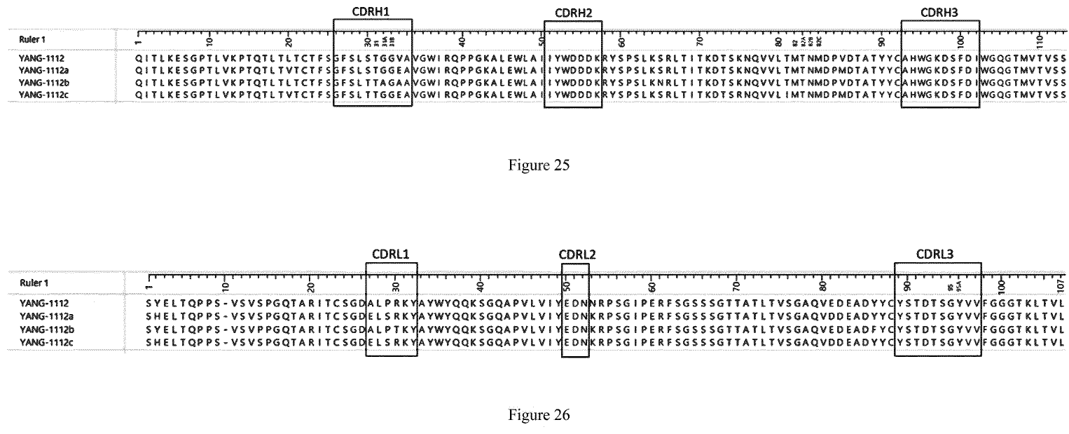

In one example, the antibody is an antibody in the YANG-1112 antibody cluster, e.g. as shown in FIGS. 25 and 26: YANG-1112a, YANG-1112b, YANG-1112c.

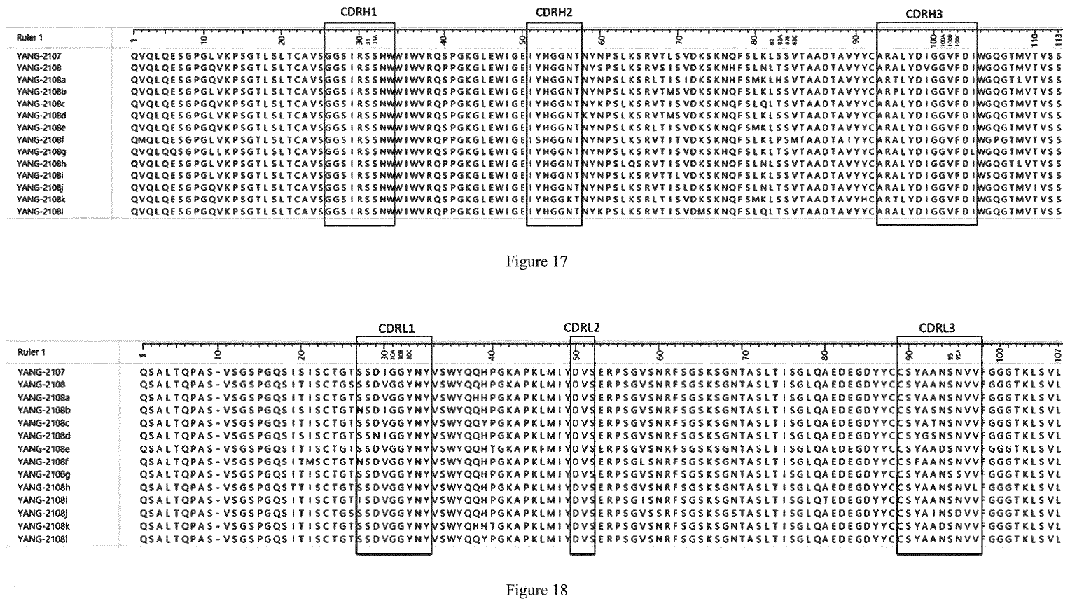

In one example, the antibody is an antibody in the YANG 2107 and 2108 antibody cluster, e.g. as shown in FIGS. 17 and 18: YANG-2108a, YANG-2108b, YANG-2108c, YANG-2108d, YANG-2108e, YANG-2108f, YANG-2108g, YANG-2108h, YANG-2108i, YANG-2108j, YANG-2108k, YANG-21081.

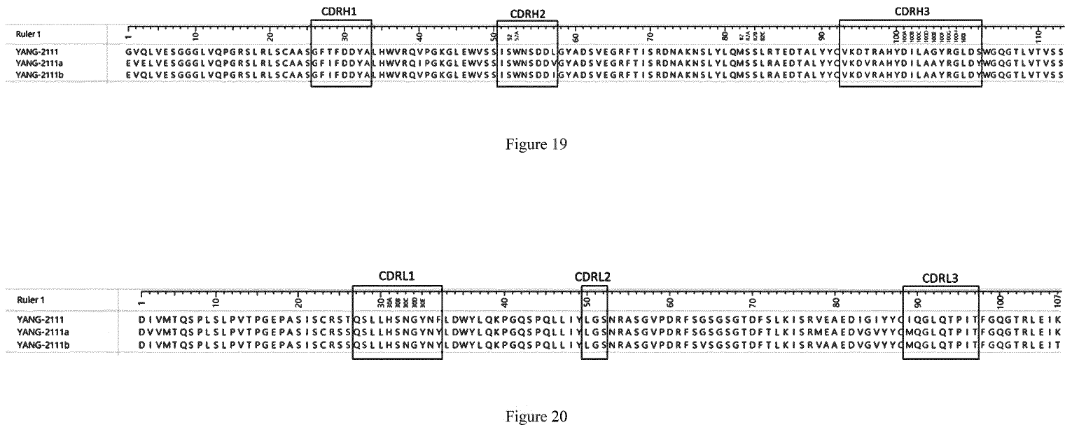

In one example, the antibody is an antibody in the YANG-2111 antibody cluster, e.g. as shown in FIGS. 19 and 20: YANG-2111a, YANG-2111b.

### Group B ‘Trimer’ Binders:

In some aspects, the antibody specifically binds the trimer form of the SARS-CoV-2 spike protein.

Such antibodies preferentially bind to the trimer form of the SARS-CoV-2 spike protein over each of the isolated RBD, isolated S1 subunit and isolated S2 subunit of the SARS-CoV-2 spike protein. Such antibodies may show at least 50 fold, at least 100 fold, at least 150 fold, or at least 200 fold higher affinity for the trimer form of the SARS-CoV-2 spike protein over each of the isolated RBD, isolated S1 subunit and isolated S2 subunit of the SARS-CoV-2 spike protein, particularly in HTRF binding assays (e.g. as set out in Example 2). Such antibodies may show at least 50 fold, at least 100 fold, at least 150 fold, or at least 200 fold higher affinity for the trimer form of the SARS-CoV-2 spike protein over each of the isolated RBD, isolated S1 subunit and isolated S2 subunit of the SARS-CoV-2 spike protein, particularly in SPR binding assays (e.g. as defined herein). Such antibodies may prevent a function of ACE2 binding to SARS-CoV-2. Such antibodies generally do not compete with ACE2 for binding to SARS-CoV-2. Such antibodies may not bind to the isolated RBD of SARS-CoV-2. Such antibodies may not bind to the isolated RBD, the isolated S1 subunit or the isolated S2 subunit of SARS-CoV-2.

Preferably, the antibody is selected from the group consisting of IMPI-030, IMPI-053, IMPI-025, IMPI-040, IMPI-007, IMPI-020, IMPI-032, IMPI-023, IMPI-039, IMPI-001, IMPI-019, IMPI-010, IMPI-008, IMPI-031, IMPI-057, IMPI-022, IMPI-035, IMPI-067 and IMPI-072. In one example, the antibody is selected from the group consisting of IMPI-030, IMPI-053, IMPI-025, IMPI-040, IMPI-007, IMPI-020, IMPI-032, IMPI-023, IMPI-039, IMPI-001 and IMPI-019 (cluster 2 in FIG. 3). In one example, the antibody is selected from the group consisting of IMPI-010, IMPI-008, IMPI-031, IMPI-057, IMPI-022 and IMPI-035 (cluster 4 in FIG. 3). In one example, the antibody is selected from the group consisting of IMPI-067 and IMPI-072 (cluster 12 in FIG. 3). In one example, the antibody is IMPI-030, IMPI-053, IMPI-025, IMPI-040, IMPI-007, IMPI-020, IMPI-032, IMPI-023, IMPI-039, IMPI-001 or IMPI-019. In one example, the antibody is IMPI-010, IMPI-008, IMPI-031, IMPI-057, IMPI-022 or IMPI-035.

In one example, the antibody is IMPI-067 or IMPI-072.

### Group C ‘S2’ Binders:

In some aspects, the antibody specifically binds to the S2 subunit of the SARS-CoV spike protein.

Provided herein are antibodies neutralise SARS-CoV-2 and specifically bind to the S2 subunit of SARS-CoV-2. Such antibodies generally do not compete with ACE2 for binding to SARS-CoV-2. Such antibodies may show high affinity for the S2 subunit e.g. KD of 10-9 M or lower. Such antibodies may be valuable as medicaments as described herein, such as in combination therapies, especially for example where they also show ADCC activity.

In one example, the antibody is selected from the group consisting of IMPI-003, IMPI-013, IMPI-063, IMPI-061, IMPI-062, IMPI-064, IMPI-065, IMPI-066, IMPI-069, IMPI-070 and IMPI-071. In one example, the antibody is selected from the group consisting of IMPI-003 and IMPI-013 (cluster 8 in FIG. 3). More preferably, the antibody is selected from the group consisting of IMPI-063, IMPI-061, IMPI-062, IMPI-064, IMPI-065, IMPI-066, IMPI-069, IMPI-070 and IMPI-071 (cluster 11 in example 3). In one example, the antibody is IMPI-003 or IMPI-013. More preferably, the antibody is IMPI-063, IMPI-061, IMPI-062, IMPI-064, IMPI-065, IMPI-066, IMPI-069, IMPI-070 or IMPI-071.

In one example, the antibody is selected from the group consisting of YANG-1201, YANG-1202, YANG-1203, YANG-1204, YANG-1205, YANG-1206, YANG-1207, YANG-2201, YANG-2202, YANG-2203, YANG-2203a, YANG-2203b, YANG-2203c, YANG-2203d, YANG-2203e, YANG-2203f, YANG-2203g, YANG-2203h, YANG-2203i, YANG-2203j, YANG-2203k, YANG-2204, YANG-2205, YANG-2206, YANG-2207, and YANG-2208.

In one example, the antibody is selected from the group consisting of YANG-2203, YANG-2204, YANG-2205, YANG-2206, YANG-2207, and YANG-2208.

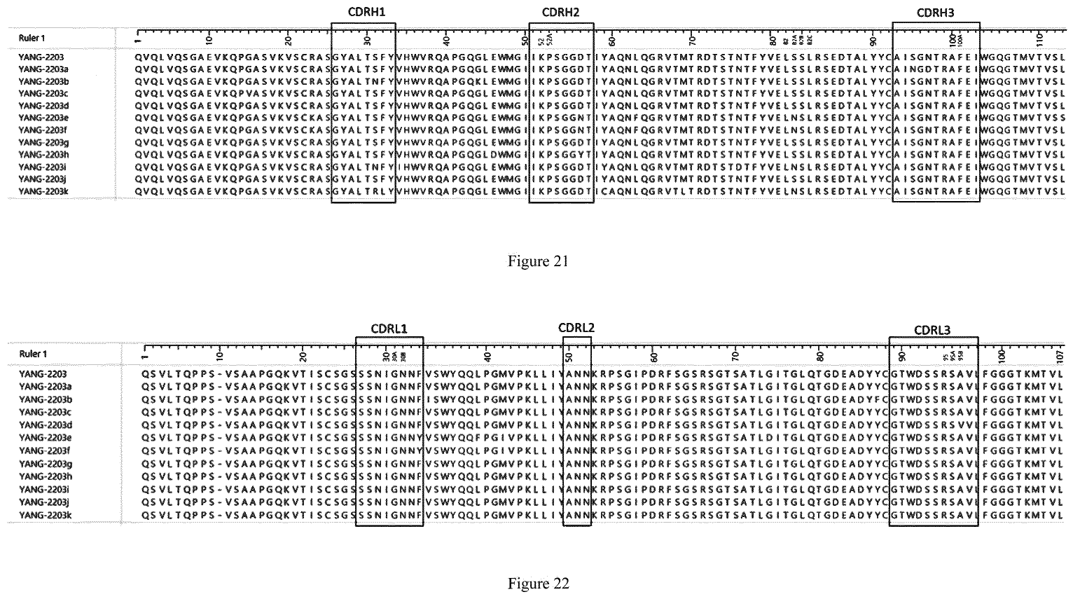

In one example, the antibody is an antibody in the YANG-2203 antibody cluster, e.g. as shown in FIGS. 21 and 22: YANG-2203a, YANG-2203b, YANG-2203c, YANG-2203d, YANG-2203e, YANG-2203f, YANG-2203g, YANG-2203h, YANG-2203i, YANG-2203j, YANG-2203k.

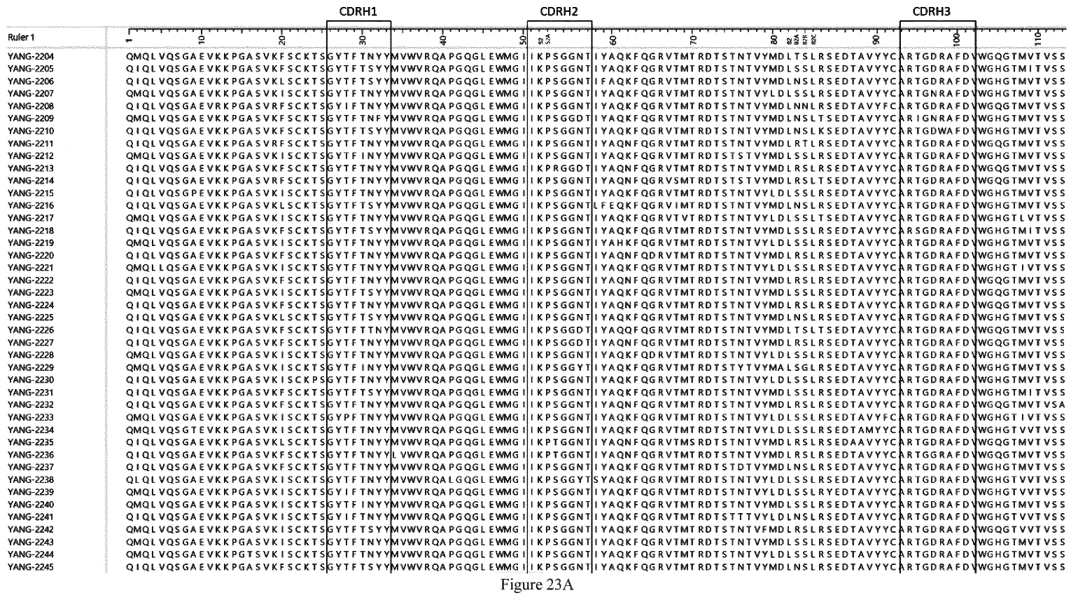

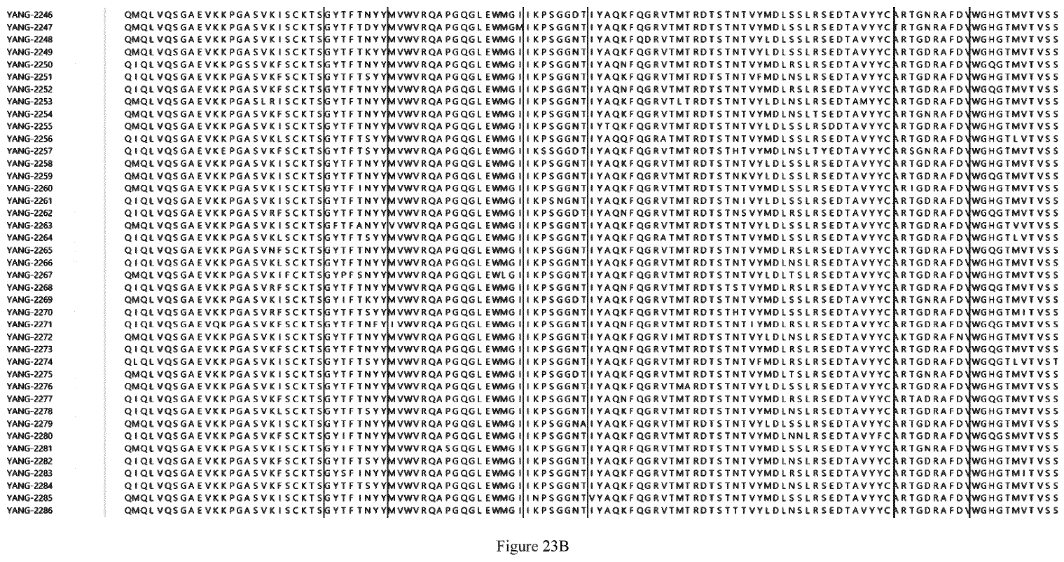

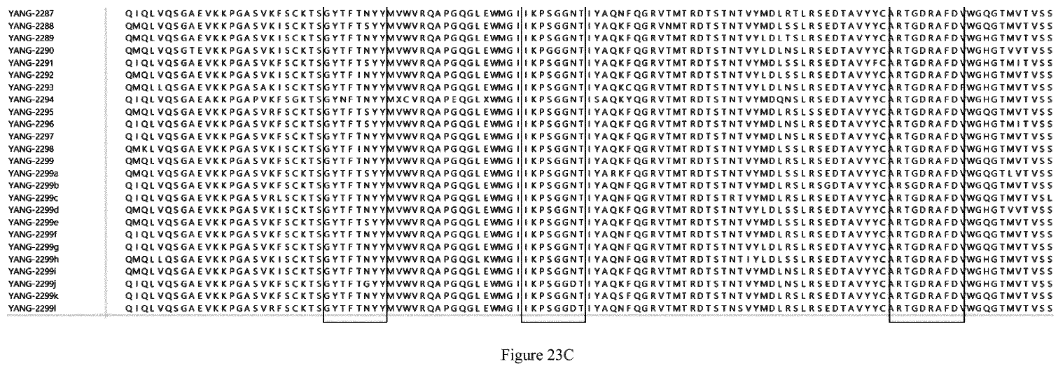

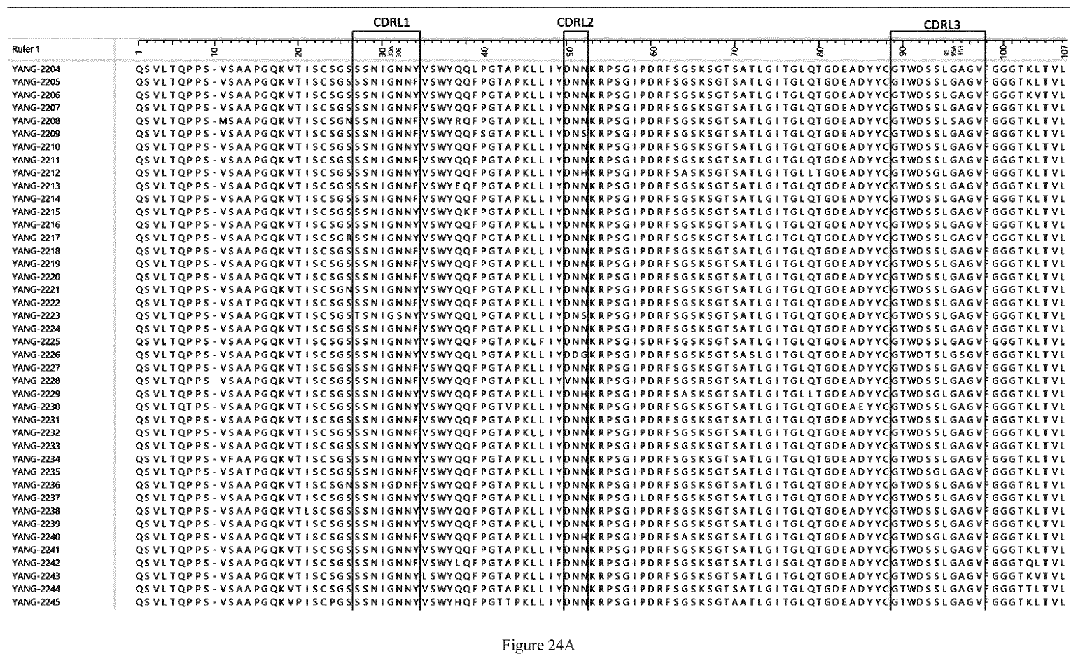

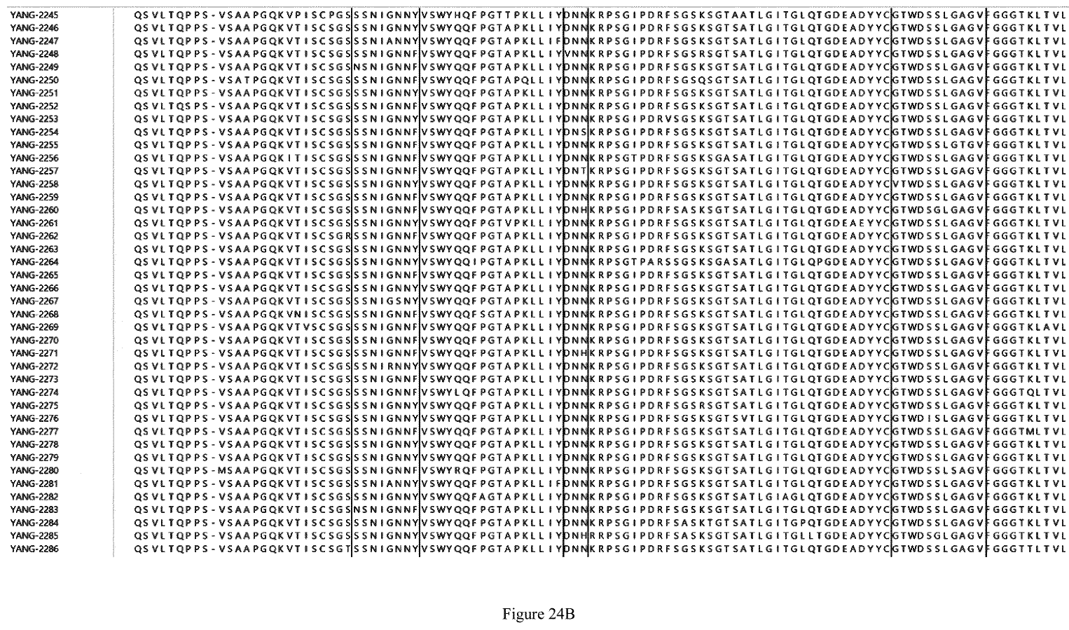

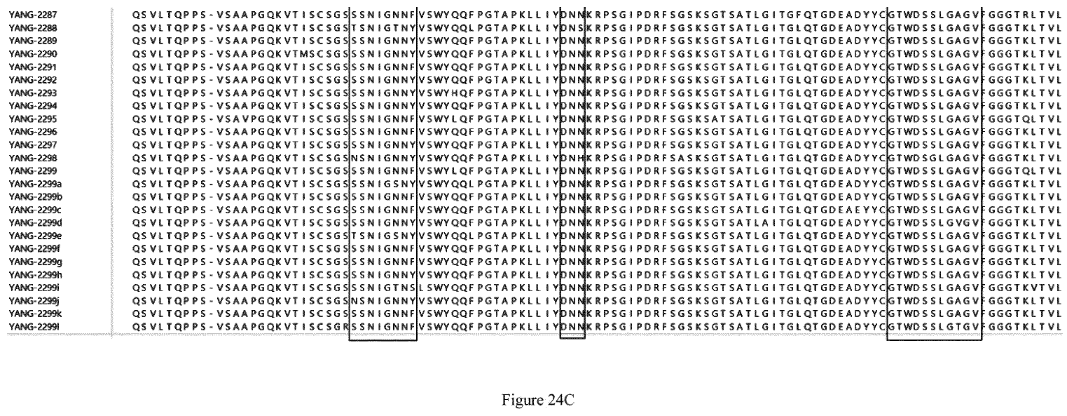

In one example, the antibody is an antibody in the YANG-2204, YANG-2205, YANG-2206, YANG-2207, and YANG-2208 antibody cluster, e.g. as shown in FIGS. 23 and 24: YANG-2209, YANG-2210, YANG-2211, YANG-2212, YANG-2213, YANG-2214, YANG-2215, YANG-2216, YANG-2217, YANG-2218, YANG-2219, YANG-2220, YANG-2221, YANG-2222, YANG-2223, YANG-2224, YANG-2225, YANG-2226, YANG-2227, YANG-2228, YANG-2229, YANG-2230, YANG-2231, YANG-2232, YANG-2233, YANG-2234, YANG-2235, YANG-2236, YANG-2237, YANG-2238, YANG-2239, YANG-2240, YANG-2241, YANG-2242, YANG-2243, YANG-2244, YANG-2245, YANG-2246, YANG-2247, YANG-2248, YANG-2249, YANG-2250, YANG-2251, YANG-2252, YANG-2253, YANG-2254, YANG-2255, YANG-2256, YANG-2257, YANG-2258, YANG-2259, YANG-2260, YANG-2261, YANG-2262, YANG-2263, YANG-2264, YANG-2265, YANG-2266, YANG-2267, YANG-2268, YANG-2269, YANG-2270, YANG-2271, YANG-2272, YANG-2273, YANG-2274, YANG-2275, YANG-2276, YANG-2277, YANG-2278, YANG-2279, YANG-2280, YANG-2281, YANG-2282, YANG-2283, YANG-2284, YANG-2285, YANG-2286, YANG-2287, YANG-2288, YANG-2289, YANG-2290, YANG-2291, YANG-2292, YANG-2293, YANG-2294, YANG-2295, YANG-2296, YANG-2297, YANG-2298, YANG-2299, YANG-2299a, YANG-2299b, YANG-2299c, YANG-2299d, YANG-2299e, YANG-2299f, YANG-2299g, YANG-2299h, YANG-2299i, YANG-2299j, YANG-2299k, YANG-22991

### Group D ‘Non-Compete’ RBD Binders:

In some aspects, the antibody specifically binds the RBD of the SARS-CoV-2 spike protein, wherein the antibody does not compete for binding to the SARS-CoV-2 spike protein with the human ACE2 receptor. Provided herein are antibodies that specifically bind to the receptor binding domain (RBD) of the S 1 subunit of SARS-CoV-2 and do not compete with ACE2 for binding to SARS-CoV-2. Such antibodies may show 0-2 fold change in neutralising activity relative to SAD S35 antibody. Such antibodies may optionally show neutralising activity. For example, the antibody may neutralise SARS-CoV-2 with an IC50 of 55 nM or lower, an IC50 of 35 nM or lower, an IC50 of 15 nM or lower, an IC50 of 10 nM or lower, or an IC50 of 3 nM or lower (e.g. as measured in a pseudovirus neutralisation assay). In other examples, the antibody may neutralise SARS-CoV-2 with an IC50 of 2 nM or greater, an IC50 of 5 nM or greater, an IC50 of 10 nM or greater, an IC50 of 30 nM or greater, or even an IC50 of 50 nM or greater (e.g. as measured in a pseudovirus neutralisation assay) and yet are still of interest as therapeutic antibodies. Such antibodies may show high affinity for the RBD e.g. KD of 10-9 M or lower. Such antibodies may result in increased binding between the RBD and the ACE2 receptor. Such antibodies may also result in destabilising of the trimer form of the SARS-CoV-2 spike protein and/or may cross-react with SARS-CoV.

In one example, the antibody is selected from the group consisting of IMPI-026, IMPI-034, IMPI-016, IMPI-050, IMPI-049, IMPI-015, IMPI-009, IMPI-011, IMPI-044, IMPI-046, IMPI-051, IMPI-024, IMPI-058, IMPI-043, IMPI-045, IMPI-027, IMPI-018, IMPI-048, IMPI-033, IMPI-014, IMPI-038 and IMPI-068. In one example, the antibody is selected from the group consisting of IMPI-026, IMPI-034, IMPI-016, IMPI-050, IMPI-049, IMPI-015, IMPI-009, IMPI-011, IMPI-044, IMPI-046, IMPI-051, IMPI-024, IMPI-058 and IMPI-043 (cluster 1 in FIG. 3). In one example, the antibody is selected from the group consisting of IMPI-045, IMPI-027, IMPI-018, IMPI-048, IMPI-033 and IMPI-014 (cluster 3 in FIG. 3). In one example, the antibody is IMPI-026, IMPI-034, IMPI-016, IMPI-050, IMPI-049, IMPI-015, IMPI-009, IMPI-011, IMPI-044, IMPI-046, IMPI-051, IMPI-024, IMPI-058 or IMPI-043. In another example, the antibody is IMPI-045, IMPI-027, IMPI-018, IMPI-048, IMPI-033 or IMPI-014.

In one example, the antibody is selected from the group consisting of YANG-1111, YANG-1102, YANG-1401, YANG-1401a, YANG-1401b, YANG-1401c, YANG-1401d, YANG-1401e, YANG-1402, YANG-1403 or YANG-2112.

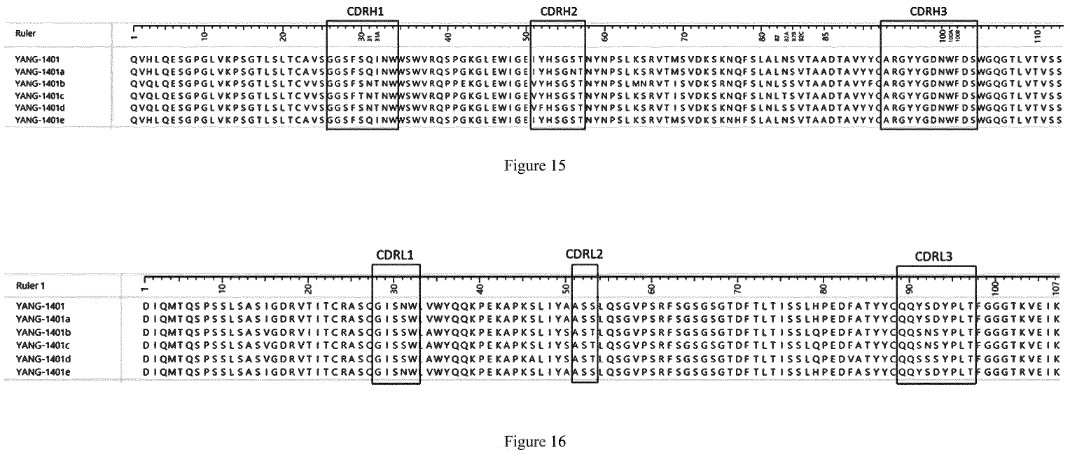

In one example, the antibody is an antibody in the YANG-1401 antibody cluster, e.g. as shown in FIGS. 15 and 16: YANG-1401a, YANG-1401b, YANG-1401c, YANG-1401d, YANG-1401e.

### Group E ‘NTD’ Binders:

In some aspects, the antibody specifically binds the NTD of the S1 sub-unit of the SARS-CoV-2 spike protein.

In one example, the antibody is selected from the group consisting of YANG-1301, YANG-1302, YANG-1303, YANG-1304, YANG-1305; YANG-2301, YANG-2302, YANG-2303, YANG-2304, YANG-2305, or YANG-2306.

### Groups A-D:

In one example the antibody is IMPI-001. In one example the antibody is IMPI-002. In one example the antibody is IMPI-003. In one example the antibody is IMPI-004. In one example the antibody is IMPI-005.

In one example the antibody is IMPI-006. In one example the antibody is IMPI-007. In one example the antibody is IMPI-008. In one example the antibody is IMPI-009. In one example the antibody is IMPI-010.

In one example the antibody is IMPI-011. In one example the antibody is IMPI-012. In one example the antibody is IMPI-013. In one example the antibody is IMPI-014. In one example the antibody is IMPI-015.

In one example the antibody is IMPI-016. In one example the antibody is IMPI-017. In one example the antibody is IMPI-018. In one example the antibody is IMPI-019. In one example the antibody is IMPI-020.

In one example the antibody is IMPI-021. In one example the antibody is IMPI-022. In one example the antibody is IMPI-023. In one example the antibody is IMPI-024. In one example the antibody is IMPI-025.

In one example the antibody is IMPI-026. In one example the antibody is IMPI-027. In one example the antibody is IMPI-028. In one example the antibody is IMPI-029. In one example the antibody is IMPI-030.

In one example the antibody is IMPI-031. In one example the antibody is IMPI-032. In one example the antibody is IMPI-033. In one example the antibody is IMPI-034. In one example the antibody is IMPI-035.

In one example the antibody is IMPI-036. In one example the antibody is IMPI-037. In one example the antibody is IMPI-038. In one example the antibody is IMPI-039. In one example the antibody is IMPI-040.

In one example the antibody is IMPI-041. In one example the antibody is IMPI-042. In one example the antibody is IMPI-043. In one example the antibody is IMPI-044. In one example the antibody is IMPI-045.

In one example the antibody is IMPI-046. In one example the antibody is IMPI-047. In one example the antibody is IMPI-048. In one example the antibody is IMPI-049. In one example the antibody is IMPI-050.

In one example the antibody is IMPI-051. In one example the antibody is IMPI-052. In one example the antibody is IMPI-053. In one example the antibody is IMPI-054. In one example the antibody is IMPI-055.

In one example the antibody is IMPI-056. In one example the antibody is IMPI-057. In one example the antibody is IMPI-058. In one example the antibody is IMPI-059. In one example the antibody is IMPI-060.

In one example the antibody is IMPI-061. In one example the antibody is IMPI-062. In one example the antibody is IMPI-063. In one example the antibody is IMPI-064. In one example the antibody is IMPI-065. In one example the antibody is IMPI-066. In one example the antibody is IMPI-067. In one example the antibody is IMPI-068. In one example the antibody is IMPI-069. In one example the antibody is IMPI-070. In one example the antibody is IMPI-071. In one example the antibody is IMPI-072.

In one example the antibody is YANG-1101.

In one example the antibody is YANG-1103.

In one example the antibody is YANG-1105.

In one example the antibody is YANG-1106.

In one example the antibody is YANG-1107.

In one example the antibody is YANG-1108.

In one example the antibody is YANG-1109.

In one example the antibody is YANG-1110.

In one example the antibody is YANG-1112.

In one example the antibody is YANG-1113.

In one example the antibody is YANG-1114.

In one example the antibody is YANG-1115.

In one example the antibody is YANG-1116.

In one example the antibody is YANG-1117.

In one example the antibody is YANG-1118.

In one example the antibody is YANG-1119.

In one example the antibody is YANG-2101.

In one example the antibody is YANG-2102.

In one example the antibody is YANG-2103.

In one example the antibody is YANG-2104.

In one example the antibody is YANG-2105.

In one example the antibody is YANG-2106.

In one example the antibody is YANG-2107.

In one example the antibody is YANG-2108.

In one example the antibody is YANG-2109.

In one example the antibody is YANG-2110.

In one example the antibody is YANG-2111.

In one example the antibody is YANG-1201.

In one example the antibody is YANG-1202.

In one example the antibody is YANG-1203.

In one example the antibody is YANG-1204.

In one example the antibody is YANG-1205.

In one example the antibody is YANG-1206.

In one example the antibody is YANG-1207.

In one example the antibody is YANG-2201.

In one example the antibody is YANG-2202.

In one example the antibody is YANG-2203.

In one example the antibody is YANG-2204.

In one example the antibody is YANG-2205.

In one example the antibody is YANG-2206.

In one example the antibody is YANG-2207.

In one example the antibody is YANG-2208.

In one example the antibody is YANG-1102.

In one example the antibody is YANG-1111.

In one example the antibody is YANG-1401.

In one example the antibody is YANG-1402.

In one example the antibody is YANG-1403.

In one example the antibody is YANG-2112.

In one embodiment, the antibody is YANG-1301.

In one embodiment, the antibody is YANG-1302.

In one embodiment, the antibody is YANG-1303.

In one embodiment, the antibody is YANG-1304.

In one embodiment, the antibody is YANG-1305.

In one embodiment, the antibody is YANG-2301.

In one embodiment, the antibody is YANG-2302.

In one embodiment, the antibody is YANG-2303.

In one embodiment, the antibody is YANG-2304.

In one embodiment, the antibody is YANG-2305.

In one embodiment, the antibody is YANG-2306.

Antibody IMPI-052 has a heavy chain variable region (VH) amino acid sequence of SEQ ID No:2, comprising the CDRH1 amino acid sequence of SEQ ID No: 3 (IMGT), the CDRH2 amino acid sequence of SEQ ID No: 4 (IMGT), and the CDRH3 amino acid sequence of SEQ ID No: 5 (IMGT). The heavy chain nucleic acid sequence of the VH domain is SEQ ID No: 1. Antibody IMPI-052 has a light chain variable region (VL) amino acid sequence of SEQ ID No: 7, comprising the CDRL1 amino acid sequence of SEQ ID No: 8 (IMGT), the CDRL2 amino acid sequence of SEQ ID No:9 (IMGT), and the CDRL3 amino acid sequence of SEQ ID No: 10 (IMGT). The light chain nucleic acid sequence of the VL domain is SEQ ID No: 6. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody IMPI-047 has a heavy chain variable region (VH) amino acid sequence of SEQ ID No: 12, comprising the CDRH1 amino acid sequence of SEQ ID No: 13 (IMGT), the CDRH2 amino acid sequence of SEQ ID No: 14 (IMGT), and the CDRH3 amino acid sequence of SEQ ID No: 15 (IMGT). The heavy chain nucleic acid sequence of the VH domain is SEQ ID No: 11. Antibody IMPI-047 has a light chain variable region (VL) amino acid sequence of SEQ ID No: 17, comprising the CDRL1 amino acid sequence of SEQ ID No: 8 (IMGT), the CDRL2 amino acid sequence of SEQ ID No: 18 (IMGT), and the CDRL3 amino acid sequence of SEQ ID No: 19 (IMGT). The light chain nucleic acid sequence of the VL domain is SEQ ID No: 16. The VH domain may be combined with any ofthe heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody IMPI-003 has a heavy chain variable region (VH) amino acid sequence of SEQ ID No: 21, comprising the CDRH1 amino acid sequence of SEQ ID No: 22 (IMGT), the CDRH2 amino acid sequence of SEQ ID No: 23 (IMGT), and the CDRH3 amino acid sequence of SEQ ID No: 24 (IMGT). The heavy chain nucleic acid sequence of the VH domain is SEQ ID No: 20. Antibody IMPI-003 has a light chain variable region (VL) amino acid sequence of SEQ ID No: 26, comprising the CDRL1 amino acid sequence of SEQ ID No: 27 (IMGT), the CDRL2 amino acid sequence of SEQ ID No: 28 (IMGT), and the CDRL3 amino acid sequence of SEQ ID No: 29 (IMGT). The light chain nucleic acid sequence of the VL domain is SEQ ID No: 25. The VH domain may be combined with any ofthe heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody IMPI-043 has a heavy chain variable region (VH) amino acid sequence of SEQ ID No: 31, comprising the CDRH1 amino acid sequence of SEQ ID No: 32 (IMGT), the CDRH2 amino acid sequence of SEQ ID No: 33 (IMGT), and the CDRH3 amino acid sequence of SEQ ID No: 34 (IMGT). The heavy chain nucleic acid sequence of the VH domain is SEQ ID No: 30. Antibody IMPI-043 has a light chain variable region (VL) amino acid sequence of SEQ ID No: 36, comprising the CDRL1 amino acid sequence of SEQ ID No: 37 (IMGT), the CDRL2 amino acid sequence of SEQ ID No: 28 (IMGT), and the CDRL3 amino acid sequence of SEQ ID No: 38 (IMGT). The light chain nucleic acid sequence of the VL domain is SEQ ID No: 35. The VH domain may be combined with any ofthe heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody IMPI-048 has a heavy chain variable region (VH) amino acid sequence of SEQ ID No: 40, comprising the CDRH1 amino acid sequence of SEQ ID No: 41 (IMGT), the CDRH2 amino acid sequence of SEQ ID No: 42 (IMGT), and the CDRH3 amino acid sequence of SEQ ID No: 43 (IMGT). The heavy chain nucleic acid sequence of the VH domain is SEQ ID No: 39. Antibody IMPI-048 has a light chain variable region (VL) amino acid sequence of SEQ ID No: 45, comprising the CDRL1 amino acid sequence of SEQ ID No: 46 (IMGT), the CDRL2 amino acid sequence of SEQ ID No: 28 (IMGT), and the CDRL3 amino acid sequence of SEQ ID No: 47 (IMGT). The light chain nucleic acid sequence of the VL domain is SEQ ID No: 44. The VH domain may be combined with any ofthe heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody IMPI-014 has a heavy chain variable region (VH) amino acid sequence of SEQ ID No: 49, comprising the CDRH1 amino acid sequence of SEQ ID No: 41 (IMGT), the CDRH2 amino acid sequence of SEQ ID No: 42 (IMGT), and the CDRH3 amino acid sequence of SEQ ID No: 43 (IMGT). The heavy chain nucleic acid sequence of the VH domain is SEQ ID No: 48. Antibody IMPI-014 has a light chain variable region (VL) amino acid sequence of SEQ ID No: 51, comprising the CDRL1 amino acid sequence of SEQ ID No: 37 (IMGT), the CDRL2 amino acid sequence of SEQ ID No: 28 (IMGT), and the CDRL3 amino acid sequence of SEQ ID No: 47 (IMGT). The light chain nucleic acid sequence of the VL domain is SEQ ID No: 50. The VH domain may be combined with any ofthe heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody IMPI-059 has a heavy chain variable region (VH) amino acid sequence of SEQ ID No: 53, comprising the CDRH1 amino acid sequence of SEQ ID No: 54 (IMGT), the CDRH2 amino acid sequence of SEQ ID No: 55 (IMGT), and the CDRH3 amino acid sequence of SEQ ID No: 56 (IMGT). The heavy chain nucleic acid sequence of the VH domain is SEQ ID No: 52. Antibody IMPI-059 has a light chain variable region (VL) amino acid sequence of SEQ ID No: 58, comprising the CDRL1 amino acid sequence of SEQ ID No: 59 (IMGT), the CDRL2 amino acid sequence of SEQ ID No: 28 (IMGT), and the CDRL3 amino acid sequence of SEQ ID No: 60 (IMGT). The light chain nucleic acid sequence of the VL domain is SEQ ID No: 57. The VH domain may be combined with any ofthe heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody IMPI-057 has a heavy chain variable region (VH) amino acid sequence of SEQ ID No: 62, comprising the CDRH1 amino acid sequence of SEQ ID No: 22 (IMGT), the CDRH2 amino acid sequence of SEQ ID No: 63 (IMGT), and the CDRH3 amino acid sequence of SEQ ID No: 64 (IMGT). The heavy chain nucleic acid sequence of the VH domain is SEQ ID No: 61. Antibody IMPI-057 has a light chain variable region (VL) amino acid sequence of SEQ ID No: 66, comprising the CDRL1 amino acid sequence of SEQ ID No: 67 (IMGT), the CDRL2 amino acid sequence of SEQ ID No: 68 (IMGT), and the CDRL3 amino acid sequence of SEQ ID No: 69 (IMGT). The light chain nucleic acid sequence of the VL domain is SEQ ID No: 65. The VH domain may be combined with any ofthe heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody IMPI-015 has a heavy chain variable region (VH) amino acid sequence of SEQ ID No: 71, comprising the CDRH1 amino acid sequence of SEQ ID No: 72 (IMGT), the CDRH2 amino acid sequence of SEQ ID No: 33 (IMGT), and the CDRH3 amino acid sequence of SEQ ID No: 34 (IMGT). The heavy chain nucleic acid sequence of the VH domain is SEQ ID No: 70. Antibody IMPI-015 has a light chain variable region (VL) amino acid sequence of SEQ ID No: 74, comprising the CDRL1 amino acid sequence of SEQ ID No: 75 (IMGT), the CDRL2 amino acid sequence of SEQ ID No: 28 (IMGT), and the CDRL3 amino acid sequence of SEQ ID No: 76 (IMGT). The light chain nucleic acid sequence of the VL domain is SEQ ID No: 73. The VH domain may be combined with any ofthe heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody IMPI-025 has a heavy chain variable region (VH) amino acid sequence of SEQ ID No: 78, comprising the CDRH1 amino acid sequence of SEQ ID No: 79 (IMGT), the CDRH2 amino acid sequence of SEQ ID No: 80 (IMGT), and the CDRH3 amino acid sequence of SEQ ID No: 81 (IMGT). The heavy chain nucleic acid sequence of the VH domain is SEQ ID No: 77. Antibody IMPI-025 has a light chain variable region (VL) amino acid sequence of SEQ ID No: 83, comprising the CDRL1 amino acid sequence of SEQ ID No: 84 (IMGT), the CDRL2 amino acid sequence of SEQ ID No: 85 (IMGT), and the CDRL3 amino acid sequence of SEQ ID No: 86 (IMGT). The light chain nucleic acid sequence of the VL domain is SEQ ID No: 82. The VH domain may be combined with any ofthe heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody IMPI-051 has a heavy chain variable region (VH) amino acid sequence of SEQ ID No: 88, comprising the CDRH1 amino acid sequence of SEQ ID No: 89 (IMGT), the CDRH2 amino acid sequence of SEQ ID No: 33 (IMGT), and the CDRH3 amino acid sequence of SEQ ID No: 34 (IMGT). The heavy chain nucleic acid sequence of the VH domain is SEQ ID No: 87. Antibody IMPI-051 has a light chain variable region (VL) amino acid sequence of SEQ ID No: 91, comprising the CDRL1 amino acid sequence of SEQ ID No: 92 (IMGT), the CDRL2 amino acid sequence of SEQ ID No: 28 (IMGT), and the CDRL3 amino acid sequence of SEQ ID No: 93 (IMGT). The light chain nucleic acid sequence of the VL domain is SEQ ID No: 90. The VH domain may be combined with any ofthe heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody IMPI-031 has a heavy chain variable region (VH) amino acid sequence of SEQ ID No: 95, comprising the CDRH1 amino acid sequence of SEQ ID No: 22 (IMGT), the CDRH2 amino acid sequence of SEQ ID No: 23 (IMGT), and the CDRH3 amino acid sequence of SEQ ID No: 96 (IMGT). The heavy chain nucleic acid sequence of the VH domain is SEQ ID No: 94. Antibody IMPI-031 has a light chain variable region (VL) amino acid sequence of SEQ ID No: 98, comprising the CDRL1 amino acid sequence of SEQ ID No: 37 (IMGT), the CDRL2 amino acid sequence of SEQ ID No: 28 (IMGT), and the CDRL3 amino acid sequence of SEQ ID No: 69 (IMGT). The light chain nucleic acid sequence of the VL domain is SEQ ID No:97. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2). Antibody IMPI-045 has a heavy chain variable region (VH) amino acid sequence of SEQ ID No: 100, comprising the CDRH1 amino acid sequence of SEQ ID No: 101 (IMGT),the CDRH2 amino acid sequence of SEQ ID No: 42 (IMGT), and the CDRH3 amino acid sequence of SEQ ID No: 43 (IMGT). The heavy chain nucleic acid sequence of the VH domain is SEQ ID No: 99. Antibody IMPI-045 has a light chain variable region (VL) amino acid sequence of SEQ ID No: 103, comprising the CDRL1 amino acid sequence of SEQ ID No: 37 (IMGT), the CDRL2 amino acid sequence of SEQ ID No: 28 (IMGT), and the CDRL3 amino acid sequence of SEQ ID No: 47 (IMGT). The light chain nucleic acid sequence of the VL domain is SEQ ID No: 102. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody IMPI-005 has a heavy chain variable region (VH) amino acid sequence of SEQ ID No: 105, comprising the CDRH1 amino acid sequence of SEQ ID No: 13 (IMGT), the CDRH2 amino acid sequence of SEQ ID No: 14 (IMGT), and the CDRH3 amino acid sequence of SEQ ID No: 106 (IMGT). The heavy chain nucleic acid sequence of the VH domain is SEQ ID No: 104. Antibody IMPI-005 has a light chain variable region (VL) amino acid sequence of SEQ ID No: 108, comprising the CDRL1 amino acid sequence of SEQ ID No: 8 (IMGT), the CDRL2 amino acid sequence of SEQ ID No: 18 (IMGT), and the CDRL3 amino acid sequence of SEQ ID No: 109 (IMGT). The light chain nucleic acid sequence of the VL domain is SEQ ID No: 107. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2). Antibody IMPI-038 has a heavy chain variable region (VH) amino acid sequence of SEQ ID No: 111, comprising the CDRH1 amino acid sequence of SEQ ID No: 112 (IMGT), the CDRH2 amino acid sequence of SEQ ID No: 113 (IMGT), and the CDRH3 amino acid sequence of SEQ ID No: 114 (IMGT). The heavy chain nucleic acid sequence of the VH domain is SEQ ID No: 110. Antibody IMPI-038 has a light chain variable region (VL) amino acid sequence of SEQ ID No: 116, comprising the CDRL1 amino acid sequence of SEQ ID No: 117 (IMGT), the CDRL2 amino acid sequence of SEQ ID No: 28 (IMGT), and the CDRL3 amino acid sequence of SEQ ID No: 118 (IMGT). The light chain nucleic acid sequence of the VL domain is SEQ ID No: 115. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2). Antibody IMPI-036 has a heavy chain variable region (VH) amino acid sequence of SEQ ID No: 120, comprising the CDRH1 amino acid sequence of SEQ ID No: 121 (IMGT), the CDRH2 amino acid sequence of SEQ ID No: 122 (IMGT), and the CDRH3 amino acid sequence of SEQ ID No: 123 (IMGT). The heavy chain nucleic acid sequence of the VH domain is SEQ ID No: 119. Antibody IMPI-036 has a light chain variable region (VL) amino acid sequence of SEQ ID No: 125, comprising the CDRL1 amino acid sequence of SEQ ID No: 126 (IMGT), the CDRL2 amino acid sequence of SEQ ID No: 127 (IMGT), and the CDRL3 amino acid sequence of SEQ ID No: 128 (IMGT). The light chain nucleic acid sequence of the VL domain is SEQ ID No: 124. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody IMPI-023 has a heavy chain variable region (VH) amino acid sequence of SEQ ID No: 130, comprising the CDRH1 amino acid sequence of SEQ ID No: 131 (IMGT), the CDRH2 amino acid sequence of SEQ ID No: 132 (IMGT), and the CDRH3 amino acid sequence of SEQ ID No: 133 (IMGT). The heavy chain nucleic acid sequence of the VH domain is SEQ ID No: 129. Antibody IMPI-023 has a light chain variable region (VL) amino acid sequence of SEQ ID No: 135, comprising the CDRL1 amino acid sequence of SEQ ID No 136 (IMGT), the CDRL2 amino acid sequence of SEQ ID No: 85 (IMGT), and the CDRL3 amino acid sequence of SEQ ID No: 86 (IMGT). The light chain nucleic acid sequence of the VL domain is SEQ ID No: 134. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody IMPI-019 has a heavy chain variable region (VH) amino acid sequence of SEQ ID No: 138, comprising the CDRH1 amino acid sequence of SEQ ID No: 139 (IMGT), the CDRH2 amino acid sequence of SEQ ID No: 140 (IMGT), and the CDRH3 amino acid sequence of SEQ ID No: 141 (IMGT). The heavy chain nucleic acid sequence of the VH domain is SEQ ID No: 137. Antibody IMPI-019 has a light chain variable region (VL) amino acid sequence of SEQ ID No: 143, comprising the CDRL1 amino acid sequence of SEQ ID No: 144 (IMGT), the CDRL2 amino acid sequence of SEQ ID No: 85 (IMGT), and the CDRL3 amino acid sequence of SEQ ID No: 86 (IMGT). The light chain nucleic acid sequence of the VL domain is SEQ ID No: 142. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody IMPI-008 has a heavy chain variable region (VH) amino acid sequence of SEQ ID No: 146, comprising the CDRH1 amino acid sequence of SEQ ID No: 22 (IMGT), the CDRH2 amino acid sequence of SEQ ID No: 23 (IMGT), and the CDRH3 amino acid sequence of SEQ ID No: 147 (IMGT). The heavy chain nucleic acid sequence of the VH domain is SEQ ID No: 145. Antibody IMPI-008 has a light chain variable region (VL) amino acid sequence of SEQ ID No: 149, comprising the CDRL1 amino acid sequence of SEQ ID No: 37 (IMGT), the CDRL2 amino acid sequence of SEQ ID No: 28 (IMGT), and the CDRL3 amino acid sequence of SEQ ID No: 69 (IMGT). The light chain nucleic acid sequence of the VL domain is SEQ ID No: 148. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody IMPI-004 has a heavy chain variable region (VH) amino acid sequence of SEQ ID No: 151, comprising the CDRH1 amino acid sequence of SEQ ID No: 112 (IMGT), the CDRH2 amino acid sequence of SEQ ID No: 152 (IMGT), and the CDRH3 amino acid sequence of SEQ ID No: 153 (IMGT). The heavy chain nucleic acid sequence of the VH domain is SEQ ID No: 150. Antibody IMPI-004 has a light chain variable region (VL) amino acid sequence of SEQ ID No: 155, comprising the CDRL1 amino acid sequence of SEQ ID No: 156 (IMGT), the CDRL2 amino acid sequence of SEQ ID No: 157 (IMGT), and the CDRL3 amino acid sequence of SEQ ID No: 158 (IMGT). The light chain nucleic acid sequence of the VL domain is SEQ ID No: 154. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody IMPI-012 has a heavy chain variable region (VH) amino acid sequence of SEQ ID No: 160, comprising the CDRH1 amino acid sequence of SEQ ID No: 161 (IMGT), the CDRH2 amino acid sequence of SEQ ID No: 4 (IMGT), and the CDRH3 amino acid sequence of SEQ ID No: 5 (IMGT). The heavy chain nucleic acid sequence of the VH domain is SEQ ID No: 159. Antibody IMPI-012 has a light chain variable region (VL) amino acid sequence of SEQ ID No: 163, comprising the CDRL1 amino acid sequence of SEQ ID No: 8 (IMGT), the CDRL2 amino acid sequence of SEQ ID No: 18 (IMGT), and the CDRL3 amino acid sequence of SEQ ID No: 164 (IMGT). The light chain nucleic acid sequence of the VL domain is SEQ ID No: 162. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody IMPI-010 has a heavy chain variable region (VH) amino acid sequence of SEQ ID No: 166, comprising the CDRH1 amino acid sequence of SEQ ID No: 22 (IMGT), the CDRH2 amino acid sequence of SEQ ID No: 23 (IMGT), and the CDRH3 amino acid sequence of SEQ ID No: 167 (IMGT). The heavy chain nucleic acid sequence of the VH domain is SEQ ID No: 165. Antibody IMPI-O10 has a light chain variable region (VL) amino acid sequence of SEQ ID No: 169, comprising the CDRL1 amino acid sequence of SEQ ID No: 67 (IMGT), the CDRL2 amino acid sequence of SEQ ID No: 28 (IMGT), and the CDRL3 amino acid sequence of SEQ ID No: 69 (IMGT). The light chain nucleic acid sequence of the VL domain is SEQ ID No: 168. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody IMPI-017 has a heavy chain variable region (VH) amino acid sequence of SEQ ID No: 171, comprising the CDRH1 amino acid sequence of SEQ ID No: 172 (IMGT), the CDRH2 amino acid sequence of SEQ ID No: 14 (IMGT), and the CDRH3 amino acid sequence of SEQ ID No: 173 (IMGT). The heavy chain nucleic acid sequence of the VH domain is SEQ ID No: 170. Antibody IMPI-017 has a light chain variable region (VL) amino acid sequence of SEQ ID No: 175, comprising the CDRL1 amino acid sequence of SEQ ID No: 176 (IMGT), the CDRL2 amino acid sequence of SEQ ID No: 18 (IMGT), and the CDRL3 amino acid sequence of SEQ ID No: 177 (IMGT). The light chain nucleic acid sequence of the VL domain is SEQ ID No: 174. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody IMPI-037 has a heavy chain variable region (VH) amino acid sequence of SEQ ID No: 179, comprising the CDRH1 amino acid sequence of SEQ ID No: 180 (IMGT), the CDRH2 amino acid sequence of SEQ ID No: 181 (IMGT), and the CDRH3 amino acid sequence of SEQ ID No: 182 (IMGT). The heavy chain nucleic acid sequence of the VH domain is SEQ ID No: 178. Antibody IMPI-037 has a light chain variable region (VL) amino acid sequence of SEQ ID No: 184, comprising the CDRL1 amino acid sequence of SEQ ID No: 185 (IMGT), the CDRL2 amino acid sequence of SEQ ID No: 18 (IMGT), and the CDRL3 amino acid sequence of SEQ ID No: 186 (IMGT). The light chain nucleic acid sequence of the VL domain is SEQ ID No: 183. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2). The sequences of antibody IMPI-037 are of particular interest in the present invention.

Antibody IMPI-022 has a heavy chain variable region (VH) amino acid sequence of SEQ ID No: 188, comprising the CDRH1 amino acid sequence of SEQ ID No: 22 (IMGT), the CDRH2 amino acid sequence of SEQ ID No: 63 (IMGT), and the CDRH3 amino acid sequence of SEQ ID No: 147 (IMGT). The heavy chain nucleic acid sequence of the VH domain is SEQ ID No: 187. Antibody IMPI-022 has a light chain variable region (VL) amino acid sequence of SEQ ID No: 190, comprising the CDRL1 amino acid sequence of SEQ ID No: 37 (IMGT), the CDRL2 amino acid sequence of SEQ ID No: 28 (IMGT), and the CDRL3 amino acid sequence of SEQ ID No: 69 (IMGT). The light chain nucleic acid sequence of the VL domain is SEQ ID No: 189. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody IMPI-058 has a heavy chain variable region (VH) amino acid sequence of SEQ ID No: 192, comprising the CDRH1 amino acid sequence of SEQ ID No: 193 (IMGT), the CDRH2 amino acid sequence of SEQ ID No: 194 (IMGT), and the CDRH3 amino acid sequence of SEQ ID No: 34 (IMGT). The heavy chain nucleic acid sequence of the VH domain is SEQ ID No: 191. Antibody IMPI-058 has a light chain variable region (VL) amino acid sequence of SEQ ID No: 196, comprising the CDRL1 amino acid sequence of SEQ ID No: 37 (IMGT), the CDRL2 amino acid sequence of SEQ ID No: 28 (IMGT), and the CDRL3 amino acid sequence of SEQ ID No: 197 (IMGT). The light chain nucleic acid sequence of the VL domain is SEQ ID No: 195. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody IMPI-024 has a heavy chain variable region (VH) amino acid sequence of SEQ ID No: 199, comprising the CDRH1 amino acid sequence of SEQ ID No: 32 (IMGT), the CDRH2 amino acid sequence of SEQ ID No: 33 (IMGT), and the CDRH3 amino acid sequence of SEQ ID No: 200 (IMGT). The heavy chain nucleic acid sequence of the VH domain is SEQ ID No: 198. Antibody IMPI-024 has a light chain variable region (VL) amino acid sequence of SEQ ID No: 202, comprising the CDRL1 amino acid sequence of SEQ ID No: 92 (IMGT), the CDRL2 amino acid sequence of SEQ ID No: 203 (IMGT), and the CDRL3 amino acid sequence of SEQ ID No: 204 (IMGT). The light chain nucleic acid sequence of the VL domain is SEQ ID No: 201. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody IMPI-039 has a heavy chain variable region (VH) amino acid sequence of SEQ ID No: 206, comprising the CDRH1 amino acid sequence of SEQ ID No: 79 (IMGT), the CDRH2 amino acid sequence of SEQ ID No: 80 (IMGT), and the CDRH3 amino acid sequence of SEQ ID No: 207 (IMGT). The heavy chain nucleic acid sequence of the VH domain is SEQ ID No: 205. Antibody IMPI-039 has a light chain variable region (VL) amino acid sequence of SEQ ID No: 209, comprising the CDRL1 amino acid sequence of SEQ ID No: 84 (IMGT), the CDRL2 amino acid sequence of SEQ ID No: 85 (IMGT), and the CDRL3 amino acid sequence of SEQ ID No: 86 (IMGT). The light chain nucleic acid sequence of the VL domain is SEQ ID No: 208. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody IMPI-020 has a heavy chain variable region (VH) amino acid sequence of SEQ ID No: 211, comprising the CDRH1 amino acid sequence of SEQ ID No: 212 (IMGT), the CDRH2 amino acid sequence of SEQ ID No: 140 (IMGT), and the CDRH3 amino acid sequence of SEQ ID No: 133 (IMGT). The heavy chain nucleic acid sequence of the VH domain is SEQ ID No: 210. Antibody IMPI-020 has a light chain variable region (VL) amino acid sequence of SEQ ID No: 214, comprising the CDRL1 amino acid sequence of SEQ ID No: 84 (IMGT), the CDRL2 amino acid sequence of SEQ ID No: 85 (IMGT), and the CDRL3 amino acid sequence of SEQ ID No: 86 (IMGT). The light chain nucleic acid sequence of the VL domain is SEQ ID No: 213. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody IMPI-053 has a heavy chain variable region (VH) amino acid sequence of SEQ ID No: 216, comprising the CDRH1 amino acid sequence of SEQ ID No: 79 (IMGT), the CDRH2 amino acid sequence of SEQ ID No: 140 (IMGT), and the CDRH3 amino acid sequence of SEQ ID No: 207 (IMGT). The heavy chain nucleic acid sequence of the VH domain is SEQ ID No: 215. Antibody IMPI-053 has a light chain variable region (VL) amino acid sequence of SEQ ID No: 218, comprising the CDRL1 amino acid sequence of SEQ ID No: 219 (IMGT), the CDRL2 amino acid sequence of SEQ ID No: 85 (IMGT), and the CDRL3 amino acid sequence of SEQ ID No: 86 (IMGT). The light chain nucleic acid sequence of the VL domain is SEQ ID No: 217. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody IMPI-021 has a heavy chain variable region (VH) amino acid sequence of SEQ ID No: 221, comprising the CDRH1 amino acid sequence of SEQ ID No: 3 (IMGT), the CDRH2 amino acid sequence of SEQ ID No: 14 (IMGT), and the CDRH3 amino acid sequence of SEQ ID No: 222 (IMGT). The heavy chain nucleic acid sequence of the VH domain is SEQ ID No: 220. Antibody IMPI-021 has a light chain variable region (VL) amino acid sequence of SEQ ID No: 224, comprising the CDRL1 amino acid sequence of SEQ ID No: 8 (IMGT), the CDRL2 amino acid sequence of SEQ ID No: 18 (IMGT), and the CDRL3 amino acid sequence of SEQ ID No: 225 (IMGT). The light chain nucleic acid sequence of the VL domain is SEQ ID No: 223. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody IMPI-032 has a heavy chain variable region (VH) amino acid sequence of SEQ ID No: 227, comprising the CDRH1 amino acid sequence of SEQ ID No: 228 (IMGT), the CDRH2 amino acid sequence of SEQ ID No: 132 (IMGT), and the CDRH3 amino acid sequence of SEQ ID No: 133 (IMGT). The heavy chain nucleic acid sequence of the VH domain is SEQ ID No: 226. Antibody IMPI-032 has a light chain variable region (VL) amino acid sequence of SEQ ID No: 230, comprising the CDRL1 amino acid sequence of SEQ ID No: 136 (IMGT), the CDRL2 amino acid sequence of SEQ ID No: 85 (IMGT), and the CDRL3 amino acid sequence of SEQ ID No: 86 (IMGT). The light chain nucleic acid sequence of the VL domain is SEQ ID No: 229. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody IMPI-001 has a heavy chain variable region (VH) amino acid sequence of SEQ ID No: 227, comprising the CDRH1 amino acid sequence of SEQ ID No: 228 (IMGT), the CDRH2 amino acid sequence of SEQ ID No: 132 (IMGT), and the CDRH3 amino acid sequence of SEQ ID No: 133 (IMGT). The heavy chain nucleic acid sequence of the VH domain is SEQ ID No: 226. Antibody IMPI-001 has a light chain variable region (VL) amino acid sequence of SEQ ID No: 232, comprising the CDRL1 amino acid sequence of SEQ ID No: 144 (IMGT), the CDRL2 amino acid sequence of SEQ ID No: 85 (IMGT), and the CDRL3 amino acid sequence of SEQ ID No: 86 (IMGT). The light chain nucleic acid sequence of the VL domain is SEQ ID No: 231. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody IMPI-041 has a heavy chain variable region (VH) amino acid sequence of SEQ ID No: 234, comprising the CDRH1 amino acid sequence of SEQ ID No 121 (IMGT), the CDRH2 amino acid sequence of SEQ ID No: 235 (IMGT), and the CDRH3 amino acid sequence of SEQ ID No: 236 (IMGT). The heavy chain nucleic acid sequence of the VH domain is SEQ ID No: 233. Antibody IMPI-041 has a light chain variable region (VL) amino acid sequence of SEQ ID No: 238, comprising the CDRL1 amino acid sequence of SEQ ID No: 126 (IMGT), the CDRL2 amino acid sequence of SEQ ID No: 239 (IMGT), and the CDRL3 amino acid sequence of SEQ ID No: 128 (IMGT). The light chain nucleic acid sequence of the VL domain is SEQ ID No: 237. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2). Antibody IMPI-029 has a heavy chain variable region (VH) amino acid sequence of SEQ ID No: 241, comprising the CDRH1 amino acid sequence of SEQ ID No: 3 (IMGT), the CDRH2 amino acid sequence of SEQ ID No: 14 (IMGT), and the CDRH3 amino acid sequence of SEQ ID No: 106 (IMGT). The heavy chain nucleic acid sequence of the VH domain is SEQ ID No: 240. Antibody IMPI-029 has a light chain variable region (VL) amino acid sequence of SEQ ID No: 108, comprising the CDRL1 amino acid sequence of SEQ ID No: 8 (IMGT), the CDRL2 amino acid sequence of SEQ ID No: 18 (IMGT), and the CDRL3 amino acid sequence of SEQ ID No: 109 (IMGT). The light chain nucleic acid sequence of the VL domain is SEQ ID No: 107. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody IMPI-009 has a heavy chain variable region (VH) amino acid sequence of SEQ ID No: 243, comprising the CDRH1 amino acid sequence of SEQ ID No: 32 (IMGT), the CDRH2 amino acid sequence of SEQ ID No: 33 (IMGT), and the CDRH3 amino acid sequence of SEQ ID No: 34 (IMGT). The heavy chain nucleic acid sequence of the VH domain is SEQ ID No: 242. Antibody X has a light chain variable region (VL) amino acid sequence of SEQ ID No: 245, comprising the CDRL1 amino acid sequence of SEQ ID No: 92 (IMGT), the CDRL2 amino acid sequence of SEQ ID No: 28 (IMGT), and the CDRL3 amino acid sequence of SEQ ID No: 246 (IMGT). The light chain nucleic acid sequence of the VL domain is SEQ ID No: 244. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody IMPI-006 has a heavy chain variable region (VH) amino acid sequence of SEQ ID No: 248, comprising the CDRH1 amino acid sequence of SEQ ID No: 249 (IMGT), the CDRH2 amino acid sequence of SEQ ID No: 250 (IMGT), and the CDRH3 amino acid sequence of SEQ ID No: 251 (IMGT). The heavy chain nucleic acid sequence of the VH domain is SEQ ID No: 247. Antibody IMPI-006 has a light chain variable region (VL) amino acid sequence of SEQ ID No: 253, comprising the CDRL1 amino acid sequence of SEQ ID No: 254 (IMGT), the CDRL2 amino acid sequence of SEQ ID No: 28 (IMGT), and the CDRL3 amino acid sequence of SEQ ID No: 255 (IMGT). The light chain nucleic acid sequence of the VL domain is SEQ ID No: 252. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2). Antibody IMPI-054 has a heavy chain variable region (VH) amino acid sequence of SEQ ID No: 257, comprising the CDRH1 amino acid sequence of SEQ ID No: 172 (IMGT), the CDRH2 amino acid sequence of SEQ ID No: 14 (IMGT), and the CDRH3 amino acid sequence of SEQ ID No: 258 (IMGT). The heavy chain nucleic acid sequence of the VH domain is SEQ ID No: 256. Antibody IMPI-054 has a light chain variable region (VL) amino acid sequence of SEQ ID No: 260, comprising the CDRL1 amino acid sequence of SEQ ID No: 261 (IMGT), the CDRL2 amino acid sequence of SEQ ID No: 262 (IMGT), and the CDRL3 amino acid sequence of SEQ ID No: 263 (IMGT). The light chain nucleic acid sequence of the VL domain is SEQ ID No: 259. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2). Antibody IMPI-044 has a heavy chain variable region (VH) amino acid sequence of SEQ ID No: 265, comprising the CDRH1 amino acid sequence of SEQ ID No: 32 (IMGT), the CDRH2 amino acid sequence of SEQ ID No: 33 (IMGT), and the CDRH3 amino acid sequence of SEQ ID No: 34 (IMGT). The heavy chain nucleic acid sequence of the VH domain is SEQ ID No: 264. Antibody IMPI-044 has a light chain variable region (VL) amino acid sequence of SEQ ID No: 267, comprising the CDRL1 amino acid sequence of SEQ ID No: 92 (IMGT), the CDRL2 amino acid sequence of SEQ ID No: 203 (IMGT), and the CDRL3 amino acid sequence of SEQ ID No: 204 (IMGT). The light chain nucleic acid sequence of the VL domain is SEQ ID No: 266. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody IMPI-002 has a heavy chain variable region (VH) amino acid sequence of SEQ ID No: 26, comprising the CDRH1 amino acid sequence of SEQ ID No: 3 (IMGT), the CDRH2 amino acid sequence of SEQ ID No: 14 (IMGT), and the CDRH3 amino acid sequence of SEQ ID No: 5 (IMGT). The heavy chain nucleic acid sequence of the VH domain is SEQ ID No: 268. Antibody IMPI-002 has a light chain variable region (VL) amino acid sequence of SEQ ID No: 271, comprising the CDRL1 amino acid sequence of SEQ ID No: 8 (IMGT), the CDRL2 amino acid sequence of SEQ ID No: 18 (IMGT), and the CDRL3 amino acid sequence of SEQ ID No: 10 (IMGT). The light chain nucleic acid sequence of the VL domain is SEQ ID No: 270. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody IMPI-027 has a heavy chain variable region (VH) amino acid sequence of SEQ ID No: 273, comprising the CDRH1 amino acid sequence of SEQ ID No: 41 (IMGT), the CDRH2 amino acid sequence of SEQ ID No: 42 (IMGT), and the CDRH3 amino acid sequence of SEQ ID No: 43 (IMGT). The heavy chain nucleic acid sequence of the VH domain is SEQ ID No: 272. Antibody IMPI-027 has a light chain variable region (VL) amino acid sequence of SEQ ID No: 275, comprising the CDRL1 amino acid sequence of SEQ ID No: 37 (IMGT), the CDRL2 amino acid sequence of SEQ ID No: 28 (IMGT), and the CDRL3 amino acid sequence of SEQ ID No: 47 (IMGT). The light chain nucleic acid sequence of the VL domain is SEQ ID No: 274. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody IMPI-011 has a heavy chain variable region (VH) amino acid sequence of SEQ ID No: 265, comprising the CDRH1 amino acid sequence of SEQ ID No: 32 (IMGT), the CDRH2 amino acid sequence of SEQ ID No: 33 (IMGT), and the CDRH3 amino acid sequence of SEQ ID No: 34 (IMGT). The heavy chain nucleic acid sequence of the VH domain is SEQ ID No: 264. Antibody IMPI-O 11 has a light chain variable region (VL) amino acid sequence of SEQ ID No: 277, comprising the CDRL1 amino acid sequence of SEQ ID No: 92 (IMGT), the CDRL2 amino acid sequence of SEQ ID No: 28 (IMGT), and the CDRL3 amino acid sequence of SEQ ID No: 76 (IMGT). The light chain nucleic acid sequence of the VL domain is SEQ ID No: 276. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2). Antibody IMPI-033 has a heavy chain variable region (VH) amino acid sequence of SEQ ID No: 279, comprising the CDRH1 amino acid sequence of SEQ ID No: 41 (IMGT), the CDRH2 amino acid sequence of SEQ ID No: 42 (IMGT), and the CDRH3 amino acid sequence of SEQ ID No: 43 (IMGT). The heavy chain nucleic acid sequence of the VH domain is SEQ ID No: 278. Antibody IMPI-033 has a light chain variable region (VL) amino acid sequence of SEQ ID No: 281, comprising the CDRL1 amino acid sequence of SEQ ID No: 37 (IMGT), the CDRL2 amino acid sequence of SEQ ID No: 28 (IMGT), and the CDRL3 amino acid sequence of SEQ ID No: 282 (IMGT). The light chain nucleic acid sequence of the VL domain is SEQ ID No: 280. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2). Antibody IMPI-055 has a heavy chain variable region (VH) amino acid sequence of SEQ ID No: 284, comprising the CDRH1 amino acid sequence of SEQ ID No: 121 (IMGT), the CDRH2 amino acid sequence of SEQ ID No: 235 (IMGT), and the CDRH3 amino acid sequence of SEQ ID No: 285 (IMGT). The heavy chain nucleic acid sequence of the VH domain is SEQ ID No: 283. Antibody IMPI-055 has a light chain variable region (VL) amino acid sequence of SEQ ID No: 287, comprising the CDRL1 amino acid sequence of SEQ ID No: 126 (IMGT), the CDRL2 amino acid sequence of SEQ ID No: 288 (IMGT), and the CDRL3 amino acid sequence of SEQ ID No: 128 (IMGT). The light chain nucleic acid sequence of the VL domain is SEQ ID No: 286. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2). Antibody IMPI-049 has a heavy chain variable region (VH) amino acid sequence of SEQ ID No: 199, comprising the CDRH1 amino acid sequence of SEQ ID No: 32 (IMGT), the CDRH2 amino acid sequence of SEQ ID No: 33 (IMGT), and the CDRH3 amino acid sequence of SEQ ID No: 200 (IMGT). The heavy chain nucleic acid sequence of the VH domain is SEQ ID No: 198. Antibody IMPI-049 has a light chain variable region (VL) amino acid sequence of SEQ ID No: 267, comprising the CDRL1 amino acid sequence of SEQ ID No: 92 (IMGT), the CDRL2 amino acid sequence of SEQ ID No: 203 (IMGT), and the CDRL3 amino acid sequence of SEQ ID No: 204 (IMGT). The light chain nucleic acid sequence of the VL domain is SEQ ID No: 289. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2). Antibody IMPI-042 has a heavy chain variable region (VH) amino acid sequence of SEQ ID No: 257, comprising the CDRH1 amino acid sequence of SEQ ID No: 172 (IMGT), the CDRH2 amino acid sequence of SEQ ID No: 14 (IMGT), and the CDRH3 amino acid sequence of SEQ ID No: 258 (IMGT). The heavy chain nucleic acid sequence of the VH domain is SEQ ID No: 290. Antibody IMPI-042 has a light chain variable region (VL) amino acid sequence of SEQ ID No: 292, comprising the CDRL1 amino acid sequence of SEQ ID No: 261 (IMGT), the CDRL2 amino acid sequence of SEQ ID No: 262 (IMGT), and the CDRL3 amino acid sequence of SEQ ID No: 263 (IMGT). The light chain nucleic acid sequence of the VL domain is SEQ ID No: 291. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2). Antibody IMPI-035 has a heavy chain variable region (VH) amino acid sequence of SEQ ID No: 294, comprising the CDRH1 amino acid sequence of SEQ ID No: 22 (IMGT), the CDRH2 amino acid sequence of SEQ ID No: 23 (IMGT), and the CDRH3 amino acid sequence of SEQ ID No: 147 (IMGT). The heavy chain nucleic acid sequence of the VH domain is SEQ ID No: 293. Antibody IMPI-035 has a light chain variable region (VL) amino acid sequence of SEQ ID No: 296, comprising the CDRL1 amino acid sequence of SEQ ID No: 37 (IMGT), the CDRL2 amino acid sequence of SEQ ID No: 28 (IMGT), and the CDRL3 amino acid sequence of SEQ ID No: 69 (IMGT). The light chain nucleic acid sequence of the VL domain is SEQ ID No: 295. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2). Antibody IMPI-028 has a heavy chain variable region (VH) amino acid sequence of SEQ ID No: 298, comprising the CDRH1 amino acid sequence of SEQ ID No: 13 (IMGT), the CDRH2 amino acid sequence of SEQ ID No: 14 (IMGT), and the CDRH3 amino acid sequence of SEQ ID No: 299 (IMGT). The heavy chain nucleic acid sequence of the VH domain is SEQ ID No: 297. Antibody IMPI-028 has a light chain variable region (VL) amino acid sequence of SEQ ID No: 301, comprising the CDRL1 amino acid sequence of SEQ ID No: 302 (IMGT), the CDRL2 amino acid sequence of SEQ ID No: 157 (IMGT), and the CDRL3 amino acid sequence of SEQ ID No: 303 (IMGT). The light chain nucleic acid sequence of the VL domain is SEQ ID No: 300. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2). Antibody IMPI-018 has a heavy chain variable region (VH) amino acid sequence of SEQ ID No: 305, comprising the CDRH1 amino acid sequence of SEQ ID No: 306 (IMGT), the CDRH2 amino acid sequence of SEQ ID No: 42 (IMGT), and the CDRH3 amino acid sequence of SEQ ID No: 307 (IMGT). The heavy chain nucleic acid sequence of the VH domain is SEQ ID No: 304. Antibody IMPI-018 has a light chain variable region (VL) amino acid sequence of SEQ ID No: 309, comprising the CDRL1 amino acid sequence of SEQ ID No: 37 (IMGT), the CDRL2 amino acid sequence of SEQ ID No: 28 (IMGT), and the CDRL3 amino acid sequence of SEQ ID No: 47 (IMGT). The light chain nucleic acid sequence of the VL domain is SEQ ID No: 308. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2). Antibody IMPI-050 has a heavy chain variable region (VH) amino acid sequence of SEQ ID No: 265, comprising the CDRH1 amino acid sequence of SEQ ID No 32 (IMGT), the CDRH2 amino acid sequence of SEQ ID No: 33 (IMGT), and the CDRH3 amino acid sequence of SEQ ID No: 34 (IMGT). The heavy chain nucleic acid sequence of the VH domain is SEQ ID No: 264. Antibody IMPI-050 has a light chain variable region (VL) amino acid sequence of SEQ ID No: 31, comprising the CDRL1 amino acid sequence of SEQ ID No: 312 (IMGT), the CDRL2 amino acid sequence of SEQ ID No: 203 (IMGT), and the CDRL3 amino acid sequence of SEQ ID No: 93 (IMGT). The light chain nucleic acid sequence of the VL domain is SEQ ID No: 310. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2). Antibody IMPI-016 has a heavy chain variable region (VH) amino acid sequence of SEQ ID No: 265, comprising the CDRH1 amino acid sequence of SEQ ID No 32 (IMGT), the CDRH2 amino acid sequence of SEQ ID No: 33 (IMGT), and the CDRH3 amino acid sequence of SEQ ID No: 34 (IMGT). The heavy chain nucleic acid sequence of the VH domain is SEQ ID No: 264. Antibody IMPI-016 has a light chain variable region (VL) amino acid sequence of SEQ ID No: 314, comprising the CDRL1 amino acid sequence of SEQ ID No: 92 (IMGT), the CDRL2 amino acid sequence of SEQ ID No: 28 (IMGT), and the CDRL3 amino acid sequence of SEQ ID No: 76 (IMGT). The light chain nucleic acid sequence of the VL domain is SEQ ID No: 313. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2). Antibody IMPI-040 has a heavy chain variable region (VH) amino acid sequence of SEQ ID No: 316, comprising the CDRH1 amino acid sequence of SEQ ID No: 131 (IMGT), the CDRH2 amino acid sequence of SEQ ID No: 140 (IMGT), and the CDRH3 amino acid sequence of SEQ ID No: 317 (IMGT). The heavy chain nucleic acid sequence of the VH domain is SEQ ID No: 315. Antibody IMPI-040 has a light chain variable region (VL) amino acid sequence of SEQ ID No: 319, comprising the CDRL1 amino acid sequence of SEQ ID No: 144 (IMGT), the CDRL2 amino acid sequence of SEQ ID No: 85 (IMGT), and the CDRL3 amino acid sequence of SEQ ID No: 86 (IMGT). The light chain nucleic acid sequence of the VL domain is SEQ ID No: 318. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2). Antibody IMPI-030 has a heavy chain variable region (VH) amino acid sequence of SEQ ID No: 78, comprising the CDRH1 amino acid sequence of SEQ ID No: 79 (IMGT), the CDRH2 amino acid sequence of SEQ ID No: 80 (IMGT), and the CDRH3 amino acid sequence of SEQ ID No: 81 (IMGT). The heavy chain nucleic acid sequence of the VH domain is SEQ ID No: 77. Antibody IMPI-030 has a light chain variable region (VL) amino acid sequence of SEQ ID No: 321, comprising the CDRL1 amino acid sequence of SEQ ID No: 84 (IMGT), the CDRL2 amino acid sequence of SEQ ID No: 85 (IMGT), and the CDRL3 amino acid sequence of SEQ ID No: 86 (IMGT). The light chain nucleic acid sequence of the VL domain is SEQ ID No: 320. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2). Antibody IMPI-034 has a heavy chain variable region (VH) amino acid sequence of SEQ ID No: 265, comprising the CDRH1 amino acid sequence of SEQ ID No: 32 (IMGT), the CDRH2 amino acid sequence of SEQ ID No: 33 (IMGT), and the CDRH3 amino acid sequence of SEQ ID No: 34 (IMGT). The heavy chain nucleic acid sequence of the VH domain is SEQ ID No: 264. Antibody IMPI-034 has a light chain variable region (VL) amino acid sequence of SEQ ID No: 323, comprising the CDRL1 amino acid sequence of SEQ ID No: 324 (IMGT), the CDRL2 amino acid sequence of SEQ ID No: 203 (IMGT), and the CDRL3 amino acid sequence of SEQ ID No: 93 (IMGT). The light chain nucleic acid sequence of the VL domain is SEQ ID No: 322. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2). Antibody IMPI-013 has a heavy chain variable region (VH) amino acid sequence of SEQ ID No: 326, comprising the CDRH1 amino acid sequence of SEQ ID No: 22 (IMGT), the CDRH2 amino acid sequence of SEQ ID No: 23 (IMGT), and the CDRH3 amino acid sequence of SEQ ID No: 327 (IMGT). The heavy chain nucleic acid sequence of the VH domain is SEQ ID No: 325. Antibody IMPI-013 has a light chain variable region (VL) amino acid sequence of SEQ ID No: 329, comprising the CDRL1 amino acid sequence of SEQ ID No: 27 (IMGT), the CDRL2 amino acid sequence of SEQ ID No: 28 (IMGT), and the CDRL3 amino acid sequence of SEQ ID No: 29 (IMGT). The light chain nucleic acid sequence of the VL domain is SEQ ID No: 328. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2). Antibody IMPI-026 has a heavy chain variable region (VH) amino acid sequence of SEQ ID No: 331, comprising the CDRH1 amino acid sequence of SEQ ID No: 32 (IMGT), the CDRH2 amino acid sequence of SEQ ID No: 33 (IMGT), and the CDRH3 amino acid sequence of SEQ ID No: 34 (IMGT). The heavy chain nucleic acid sequence of the VH domain is SEQ ID No: 330. Antibody IMPI-026 has a light chain variable region (VL) amino acid sequence of SEQ ID No: 333, comprising the CDRL1 amino acid sequence of SEQ ID No: 37 (IMGT), the CDRL2 amino acid sequence of SEQ ID No: 28 (IMGT), and the CDRL3 amino acid sequence of SEQ ID No: 76 (IMGT). The light chain nucleic acid sequence of the VL domain is SEQ ID No: 332. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2). Antibody IMPI-007 has a heavy chain variable region (VH) amino acid sequence of SEQ ID No: 335, comprising the CDRH1 amino acid sequence of SEQ ID No: 336 (IMGT), the CDRH2 amino acid sequence of SEQ ID No: 140 (IMGT), and the CDRH3 amino acid sequence of SEQ ID No: 337 (IMGT). The heavy chain nucleic acid sequence of the VH domain is SEQ ID No: 334. Antibody IMPI-007 has a light chain variable region (VL) amino acid sequence of SEQ ID No: 339, comprising the CDRL1 amino acid sequence of SEQ ID No: 84 (IMGT), the CDRL2 amino acid sequence of SEQ ID No: 85 (IMGT), and the CDRL3 amino acid sequence of SEQ ID No: 86 (IMGT). The light chain nucleic acid sequence of the VL domain is SEQ ID No: 338. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2). Antibody IMPI-046 has a heavy chain variable region (VH) amino acid sequence of SEQ ID No: 341, comprising the CDRH1 amino acid sequence of SEQ ID No: 342 (IMGT), the CDRH2 amino acid sequence of SEQ ID No: 33 (IMGT), and the CDRH3 amino acid sequence of SEQ ID No: 34 (IMGT). The heavy chain nucleic acid sequence of the VH domain is SEQ ID No: 340. Antibody IMPI-046 has a light chain variable region (VL) amino acid sequence of SEQ ID No: 344, comprising the CDRL1 amino acid sequence of SEQ ID No: 37 (IMGT), the CDRL2 amino acid sequence of SEQ ID No: 28 (IMGT), and the CDRL3 amino acid sequence of SEQ ID No: 38 (IMGT). The light chain nucleic acid sequence of the VL domain is SEQ ID No: 343. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody IMPI-060 has a heavy chain variable region (VH) amino acid sequence of SEQ ID No: 221, comprising the CDRH1 amino acid sequence of SEQ ID No: 3 (IMGT), the CDRH2 amino acid sequence of SEQ ID No: 14 (IMGT), and the CDRH3 amino acid sequence of SEQ ID No: 222 (IMGT). The heavy chain nucleic acid sequence of the VH domain is SEQ ID No: 220. Antibody IMPI-060 has a light chain variable region (VL) amino acid sequence of SEQ ID No: 346, comprising the CDRL1 amino acid sequence of SEQ ID No: 8 (IMGT), the CDRL2 amino acid sequence of SEQ ID No: 18 (IMGT), and the CDRL3 amino acid sequence of SEQ ID No: 347 (IMGT). The light chain nucleic acid sequence of the VL domain is SEQ ID No: 345. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody IMPI-056 has a heavy chain variable region (VH) amino acid sequence of SEQ ID No: 349, comprising the CDRH1 amino acid sequence of SEQ ID No: 172 (IMGT), the CDRH2 amino acid sequence of SEQ ID No: 14 (IMGT), and the CDRH3 amino acid sequence of SEQ ID No: 106 (IMGT). The heavy chain nucleic acid sequence of the VH domain is SEQ ID No: 348. Antibody IMPI-056 has a light chain variable region (VL) amino acid sequence of SEQ ID No: 351, comprising the CDRL1 amino acid sequence of SEQ ID No: 8 (IMGT), the CDRL2 amino acid sequence of SEQ ID No: 18 (IMGT), and the CDRL3 amino acid sequence of SEQ ID No: 10 (IMGT). The light chain nucleic acid sequence of the VL domain is SEQ ID No: 350. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody IMPI-061 has a heavy chain variable region (VH) amino acid sequence of SEQ ID No: 353, comprising the CDRH1 amino acid sequence of SEQ ID No: 354 (IMGT), the CDRH2 amino acid sequence of SEQ ID No: 355 (IMGT), and the CDRH3 amino acid sequence of SEQ ID No: 356 (IMGT). The heavy chain nucleic acid sequence of the VH domain is SEQ ID No: 352. Antibody IMPI-061 has a light chain variable region (VL) amino acid sequence of SEQ ID No: 358, comprising the CDRL1 amino acid sequence of SEQ ID No: 359 (IMGT), the CDRL2 amino acid sequence of SEQ ID No: 157 (IMGT), and the CDRL3 amino acid sequence of SEQ ID No: 360 (IMGT). The light chain nucleic acid sequence of the VL domain is SEQ ID No: 357. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody IMPI-062 has a heavy chain variable region (VH) amino acid sequence of SEQ ID No: 353, comprising the CDRH1 amino acid sequence of SEQ ID No: 354 (IMGT), the CDRH2 amino acid sequence of SEQ ID No: 355 (IMGT), and the CDRH3 amino acid sequence of SEQ ID No: 356 (IMGT). The heavy chain nucleic acid sequence of the VH domain is SEQ ID No: 361. Antibody IMPI-062 has a light chain variable region (VL) amino acid sequence of SEQ ID No: 363, comprising the CDRL1 amino acid sequence of SEQ ID No: 364 (IMGT), the CDRL2 amino acid sequence of SEQ ID No: 157 (IMGT), and the CDRL3 amino acid sequence of SEQ ID No: 360 (IMGT). The light chain nucleic acid sequence of the VL domain is SEQ ID No: 362. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody IMPI-063 has a heavy chain variable region (VH) amino acid sequence of SEQ ID No: 366, comprising the CDRH1 amino acid sequence of SEQ ID No: 354 (IMGT), the CDRH2 amino acid sequence of SEQ ID No: 355 (IMGT), and the CDRH3 amino acid sequence of SEQ ID No: 356 (IMGT). The heavy chain nucleic acid sequence of the VH domain is SEQ ID No: 365. Antibody IMPI-063 has a light chain variable region (VL) amino acid sequence of SEQ ID No: 368, comprising the CDRL1 amino acid sequence of SEQ ID No: 369 (IMGT), the CDRL2 amino acid sequence of SEQ ID No: 157 (IMGT), and the CDRL3 amino acid sequence of SEQ ID No: 360 (IMGT). The light chain nucleic acid sequence of the VL domain is SEQ ID No: 367. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody IMPI-064 has a heavy chain variable region (VH) amino acid sequence of SEQ ID No: 353, comprising the CDRH1 amino acid sequence of SEQ ID No: 354 (IMGT), the CDRH2 amino acid sequence of SEQ ID No: 355 (IMGT), and the CDRH3 amino acid sequence of SEQ ID No: 356 (IMGT). The heavy chain nucleic acid sequence of the VH domain is SEQ ID No: 370. Antibody IMPI-064 has a light chain variable region (VL) amino acid sequence of SEQ ID No: 372, comprising the CDRL1 amino acid sequence of SEQ ID No: 364 (IMGT), the CDRL2 amino acid sequence of SEQ ID No: 157 (IMGT), and the CDRL3 amino acid sequence of SEQ ID No: 373 (IMGT). The light chain nucleic acid sequence of the VL domain is SEQ ID No: 371. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody IMPI-065 has a heavy chain variable region (VH) amino acid sequence of SEQ ID No: 353, comprising the CDRH1 amino acid sequence of SEQ ID No: 354 (IMGT), the CDRH2 amino acid sequence of SEQ ID No: 355 (IMGT), and the CDRH3 amino acid sequence of SEQ ID No: 356 (IMGT). The heavy chain nucleic acid sequence of the VH domain is SEQ ID No: 352. Antibody IMPI-065 has a light chain variable region (VL) amino acid sequence of SEQ ID No: 375, comprising the CDRL1 amino acid sequence of SEQ ID No: 364 (IMGT), the CDRL2 amino acid sequence of SEQ ID No: 157 (IMGT), and the CDRL3 amino acid sequence of SEQ ID No: 376 (IMGT). The light chain nucleic acid sequence of the VL domain is SEQ ID No: 374. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody IMPI-066 has a heavy chain variable region (VH) amino acid sequence of SEQ ID No: 378, comprising the CDRH1 amino acid sequence of SEQ ID No: 354 (IMGT), the CDRH2 amino acid sequence of SEQ ID No: 379 (IMGT), and the CDRH3 amino acid sequence of SEQ ID No: 356 (IMGT). The heavy chain nucleic acid sequence of the VH domain is SEQ ID No: 377. Antibody IMPI-066 has a light chain variable region (VL) amino acid sequence of SEQ ID No: 381, comprising the CDRL1 amino acid sequence of SEQ ID No: 382 (IMGT), the CDRL2 amino acid sequence of SEQ ID No: 157 (IMGT), and the CDRL3 amino acid sequence of SEQ ID No: 383 (IMGT). The light chain nucleic acid sequence of the VL domain is SEQ ID No: 380. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody IMPI-067 has a heavy chain variable region (VH) amino acid sequence of SEQ ID No: 385, comprising the CDRH1 amino acid sequence of SEQ ID No: 386 (IMGT), the CDRH2 amino acid sequence of SEQ ID No: 387 (IMGT), and the CDRH3 amino acid sequence of SEQ ID No: 388 (IMGT). The heavy chain nucleic acid sequence of the VH domain is SEQ ID No: 384. Antibody IMPI-067 has a light chain variable region (VL) amino acid sequence of SEQ ID No: 390, comprising the CDRL1 amino acid sequence of SEQ ID No: 391 (IMGT), the CDRL2 amino acid sequence of SEQ ID No: 157 (IMGT), and the CDRL3 amino acid sequence of SEQ ID No: 392 (IMGT). The light chain nucleic acid sequence of the VL domain is SEQ ID No: 389. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody IMPI-068 has a heavy chain variable region (VH) amino acid sequence of SEQ ID No: 394, comprising the CDRH1 amino acid sequence of SEQ ID No: 395 (IMGT), the CDRH2 amino acid sequence of SEQ ID No: 113 (IMGT), and the CDRH3 amino acid sequence of SEQ ID No: 396 (IMGT). The heavy chain nucleic acid sequence of the VH domain is SEQ ID No: 393. Antibody IMPI-068 has a light chain variable region (VL) amino acid sequence of SEQ ID No: 398, comprising the CDRL1 amino acid sequence of SEQ ID No: 37 (IMGT), the CDRL2 amino acid sequence of SEQ ID No: 28 (IMGT), and the CDRL3 amino acid sequence of SEQ ID No: 399 (IMGT). The light chain nucleic acid sequence of the VL domain is SEQ ID No: 397. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody IMPI-069 has a heavy chain variable region (VH) amino acid sequence of SEQ ID No: 401, comprising the CDRH1 amino acid sequence of SEQ ID No: 354 (IMGT), the CDRH2 amino acid sequence of SEQ ID No: 355 (IMGT), and the CDRH3 amino acid sequence of SEQ ID No: 356 (IMGT). The heavy chain nucleic acid sequence of the VH domain is SEQ ID No: 400. Antibody IMPI-069 has a light chain variable region (VL) amino acid sequence of SEQ ID No: 403, comprising the CDRL1 amino acid sequence of SEQ ID No: 404 (IMGT), the CDRL2 amino acid sequence of SEQ ID No: 157 (IMGT), and the CDRL3 amino acid sequence of SEQ ID No: 360 (IMGT). The light chain nucleic acid sequence of the VL domain is SEQ ID No: 402. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody IMPI-070 has a heavy chain variable region (VH) amino acid sequence of SEQ ID No: 353, comprising the CDRH1 amino acid sequence of SEQ ID No: 354 (IMGT), the CDRH2 amino acid sequence of SEQ ID No: 355 (IMGT), and the CDRH3 amino acid sequence of SEQ ID No: 356 (IMGT). The heavy chain nucleic acid sequence of the VH domain is SEQ ID No: 370. Antibody IMPI-070 has a light chain variable region (VL) amino acid sequence of SEQ ID No: 406, comprising the CDRL1 amino acid sequence of SEQ ID No: 407 (IMGT), the CDRL2 amino acid sequence of SEQ ID No: 157 (IMGT), and the CDRL3 amino acid sequence of SEQ ID No: 408 (IMGT). The light chain nucleic acid sequence of the VL domain is SEQ ID No: 405. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody IMPI-071 has a heavy chain variable region (VH) amino acid sequence of SEQ ID No: 353, comprising the CDRH1 amino acid sequence of SEQ ID No: 354 (IMGT), the CDRH2 amino acid sequence of SEQ ID No: 355 (IMGT), and the CDRH3 amino acid sequence of SEQ ID No: 356 (IMGT). The heavy chain nucleic acid sequence of the VH domain is SEQ ID No: 370. Antibody IMPI-071 has a light chain variable region (VL) amino acid sequence of SEQ ID No: 410, comprising the CDRL1 amino acid sequence of SEQ ID No: 364 (IMGT), the CDRL2 amino acid sequence of SEQ ID No: 157 (IMGT), and the CDRL3 amino acid sequence of SEQ ID No: 360 (IMGT). The light chain nucleic acid sequence of the VL domain is SEQ ID No: 409. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody IMPI-072 has a heavy chain variable region (VH) amino acid sequence of SEQ ID No: 412, comprising the CDRH1 amino acid sequence of SEQ ID No: 413 (IMGT), the CDRH2 amino acid sequence of SEQ ID No: 387 (IMGT), and the CDRH3 amino acid sequence of SEQ ID No: 388 (IMGT). The heavy chain nucleic acid sequence of the VH domain is SEQ ID No: 411. Antibody IMPI-072 has a light chain variable region (VL) amino acid sequence of SEQ ID No: 415, comprising the CDRL1 amino acid sequence of SEQ ID No: 391 (IMGT), the CDRL2 amino acid sequence of SEQ ID No: 157 (IMGT), and the CDRL3 amino acid sequence of SEQ ID No: 416 (IMGT). The light chain nucleic acid sequence of the VL domain is SEQ ID No: 414. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

The sequences of YANG antibodies provided herein (particularly RBD-binders YANG-1401, YANG-1112, YANG-2107, YANG-2108, and YANG-2111; and S2-binders YANG-2203, YANG-2204, YANG-2205, YANG-2206, YANG-2207, and YANG-2208) are of particular interest in the present invention.

Antibody YANG-1401 has a heavy chain variable region (VH) amino acid sequence of SEQ ID No: 777, comprising the CDRH1 amino acid sequence of SEQ ID No: 778 (IMGT), the CDRH2 amino acid sequence of SEQ ID No: 779 (IMGT), and the CDRH3 amino acid sequence of SEQ ID No: 780 (IMGT). The heavy chain nucleic acid sequence of the VH domain is SEQ ID No: 776. Antibody YANG-1401 has a light chain variable region (VL) amino acid sequence of SEQ ID No: 782, comprising the CDRL1 amino acid sequence of SEQ ID No: 783 (IMGT), the CDRL2 amino acid sequence of SEQ ID No: 18 (IMGT), and the CDRL3 amino acid sequence of SEQ ID No: 784 (IMGT). The light chain nucleic acid sequence of the VL domain is SEQ ID No: 781. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody YANG-1112 has a heavy chain variable region (VH) amino acid sequence of SEQ ID No: 571, comprising the CDRH1 amino acid sequence of SEQ ID No: 572 (IMGT), the CDRH2 amino acid sequence of SEQ ID No: 573 (IMGT), and the CDRH3 amino acid sequence of SEQ ID No: 574 (IMGT). The heavy chain nucleic acid sequence of the VH domain is SEQ ID No: 570. Antibody YANG-1112 has a light chain variable region (VL) amino acid sequence of SEQ ID No: 576, comprising the CDRL1 amino acid sequence of SEQ ID No: 577 (IMGT), the CDRL2 amino acid sequence of SEQ ID No: 578 (IMGT), and the CDRL3 amino acid sequence of SEQ ID No: 579 (IMGT). The light chain nucleic acid sequence of the VL domain is SEQ ID No: 575. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody YANG-2107 has a heavy chain variable region (VH) amino acid sequence of SEQ ID No: 898, comprising the CDRH1 amino acid sequence of SEQ ID No: 899 (IMGT), the CDRH2 amino acid sequence of SEQ ID No: 900 (IMGT), and the CDRH3 amino acid sequence of SEQ ID No: 901 (IMGT). The heavy chain nucleic acid sequence of the VH domain is SEQ ID No: 897. Antibody YANG-2107 has a light chain variable region (VL) amino acid sequence of SEQ ID No: 903, comprising the CDRL1 amino acid sequence of SEQ ID No: 904 (IMGT), the CDRL2 amino acid sequence of SEQ ID No: 203 (IMGT), and the CDRL3 amino acid sequence of SEQ ID No: 905 (IMGT). The light chain nucleic acid sequence of the VL domain is SEQ ID No: 902. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody YANG-2108 has a heavy chain variable region (VH) amino acid sequence of SEQ ID No: 907, comprising the CDRH1 amino acid sequence of SEQ ID No: 908 (IMGT), the CDRH2 amino acid sequence of SEQ ID No: 909 (IMGT), and the CDRH3 amino acid sequence of SEQ ID No: 910 (IMGT). The heavy chain nucleic acid sequence of the VH domain is SEQ ID No: 906. Antibody YANG-2108 has a light chain variable region (VL) amino acid sequence of SEQ ID No: 912, comprising the CDRL1 amino acid sequence of SEQ ID No: 913 (IMGT), the CDRL2 amino acid sequence of SEQ ID No: 203 (IMGT), and the CDRL3 amino acid sequence of SEQ ID No: 914 (IMGT). The light chain nucleic acid sequence of the VL domain is SEQ ID No: 911. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2). Antibody YANG-2111 has a heavy chain variable region (VH) amino acid sequence of SEQ ID No: 1045, comprising the CDRH1 amino acid sequence of SEQ ID No: 1046 (IMGT), the CDRH2 amino acid sequence of SEQ ID No: 1047 (IMGT), and the CDRH3 amino acid sequence of SEQ ID No: 1048 (IMGT). The heavy chain nucleic acid sequence of the VH domain is SEQ ID No: 1044. Antibody YANG-2111 has a light chain variable region (VL) amino acid sequence of SEQ ID No: 1050, comprising the CDRL1 amino acid sequence of SEQ ID No: 1051 (IMGT), the CDRL2 amino acid sequence of SEQ ID No: 1052 (IMGT), and the CDRL3 amino acid sequence of SEQ ID No: 1053 (IMGT). The light chain nucleic acid sequence of the VL domain is SEQ ID No: 1049. The VH domain may be combined with any of the heavy chain constant region sequences described herein (the nucleotide and corresponding amino acid sequences of which are set out in Table 2). The VL domain may be combined with any ofthe light chain constant region sequences described herein (the nucleotide and corresponding amino acid sequences of which are set out in Table 2).

Antibody YANG-2203 has a heavy chain variable region (VH) amino acid sequence of SEQ ID No: 1105, comprising the CDRH1 amino acid sequence of SEQ ID No: 1106 (IMGT), the CDRH2 amino acid sequence of SEQ ID No: 1107 (IMGT), and the CDRH3 amino acid sequence of SEQ ID No: 1108 (IMGT). The heavy chain nucleic acid sequence of the VH domain is SEQ ID No: 1104. Antibody YANG-2203 has a light chain variable region (VL) amino acid sequence of SEQ ID No: 1110, comprising the CDRL1 amino acid sequence of SEQ ID No: 1111 (IMGT), the CDRL2 amino acid sequence of SEQ ID No: 1112 (IMGT), and the CDRL3 amino acid sequence of SEQ ID No: 1113 (IMGT). The light chain nucleic acid sequence of the VL domain is SEQ ID No: 1109. The VH domain may be combined with any of the heavy chain constant region sequences described herein (the nucleotide and corresponding amino acid sequences of which are set out in Table 2). The VL domain may be combined with any ofthe light chain constant region sequences described herein (the nucleotide and corresponding amino acid sequences of which are set out in Table 2).

Antibody YANG-2204 has a heavy chain variable region (VH) amino acid sequence of SEQ ID No: 1225, comprising the CDRH1 amino acid sequence of SEQ ID No: 1226 (IMGT), the CDRH2 amino acid sequence of SEQ ID No: 1227 (IMGT), and the CDRH3 amino acid sequence of SEQ ID No: 1228 (IMGT). The heavy chain nucleic acid sequence of the VH domain is SEQ ID No: 1224. Antibody YANG-2204 has a light chain variable region (VL) amino acid sequence of SEQ ID No: 1230, comprising the CDRL1 amino acid sequence of SEQ ID No: 1231 (IMGT), the CDRL2 amino acid sequence of SEQ ID No: 1232 (IMGT), and the CDRL3 amino acid sequence of SEQ ID No: 1233 (IMGT). The light chain nucleic acid sequence of the VL domain is SEQ ID No: 1229. The VH domain may be combined with any of the heavy chain constant region sequences described herein (the nucleotide and corresponding amino acid sequences of which are set out in Table 2). The VL domain may be combined with any ofthe light chain constant region sequences described herein (the nucleotide and corresponding amino acid sequences of which are set out in Table 2).

Antibody YANG-2205 has a heavy chain variable region (VH) amino acid sequence of SEQ ID No: 1235, comprising the CDRH1 amino acid sequence of SEQ ID No: 1236 (IMGT), the CDRH2 amino acid sequence of SEQ ID No: 1237 (IMGT), and the CDRH3 amino acid sequence of SEQ ID No: 1238 (IMGT). The heavy chain nucleic acid sequence of the VH domain is SEQ ID No: 1234. Antibody YANG-2205 has a light chain variable region (VL) amino acid sequence of SEQ ID No: 1240, comprising the CDRL1 amino acid sequence of SEQ ID No: 1241 (IMGT), the CDRL2 amino acid sequence of SEQ ID No: 1242 (IMGT), and the CDRL3 amino acid sequence of SEQ ID No: 1243 (IMGT). The light chain nucleic acid sequence of the VL domain is SEQ ID No: 1239. The VH domain may be combined with any of the heavy chain constant region sequences described herein (the nucleotide and corresponding amino acid sequences of which are set out in Table 2). The VL domain may be combined with any ofthe light chain constant region sequences described herein (the nucleotide and corresponding amino acid sequences of which are set out in Table 2).

Antibody YANG-2206 has a heavy chain variable region (VH) amino acid sequence of SEQ ID No: 1245, comprising the CDRH1 amino acid sequence of SEQ ID No: 1246 (IMGT), the CDRH2 amino acid sequence of SEQ ID No: 1247 (IMGT), and the CDRH3 amino acid sequence of SEQ ID No: 1248 (IMGT). The heavy chain nucleic acid sequence of the VH domain is SEQ ID No: 1244. Antibody YANG-2206 has a light chain variable region (VL) amino acid sequence of SEQ ID No: 1250, comprising the CDRL1 amino acid sequence of SEQ ID No: 1251 (IMGT), the CDRL2 amino acid sequence of SEQ ID No: 1252 (IMGT), and the CDRL3 amino acid sequence of SEQ ID No: 1253 (IMGT). The light chain nucleic acid sequence of the VL domain is SEQ ID No: 1249. The VH domain may be combined with any of the heavy chain constant region sequences described herein (the nucleotide and corresponding amino acid sequences of which are set out in Table 2). The VL domain may be combined with any ofthe light chain constant region sequences described herein (the nucleotide and corresponding amino acid sequences of which are set out in Table 2).

Antibody YANG-2207 has a heavy chain variable region (VH) amino acid sequence of SEQ ID No: 1255, comprising the CDRH1 amino acid sequence of SEQ ID No: 1256 (IMGT), the CDRH2 amino acid sequence of SEQ ID No: 1257 (IMGT), and the CDRH3 amino acid sequence of SEQ ID No: 1258 (IMGT). The heavy chain nucleic acid sequence of the VH domain is SEQ ID No: 1254. Antibody YANG-2207 has a light chain variable region (VL) amino acid sequence of SEQ ID No: 1260, comprising the CDRL1 amino acid sequence of SEQ ID No: 1261 (IMGT), the CDRL2 amino acid sequence of SEQ ID No: 1262 (IMGT), and the CDRL3 amino acid sequence of SEQ ID No: 1263 (IMGT). The light chain nucleic acid sequence of the VL domain is SEQ ID No: 1259. The VH domain may be combined with any of the heavy chain constant region sequences described herein (the nucleotide and corresponding amino acid sequences of which are set out in Table 2). The VL domain may be combined with any ofthe light chain constant region sequences described herein (the nucleotide and corresponding amino acid sequences of which are set out in Table 2).

Antibody YANG-2208 has a heavy chain variable region (VH) amino acid sequence of SEQ ID No: 1265, comprising the CDRH1 amino acid sequence of SEQ ID No: 1266 (IMGT), the CDRH2 amino acid sequence of SEQ ID No: 1267 (IMGT), and the CDRH3 amino acid sequence of SEQ ID No: 1268 (IMGT). The heavy chain nucleic acid sequence of the VH domain is SEQ ID No: 1264. Antibody YANG-2208 has a light chain variable region (VL) amino acid sequence of SEQ ID No: 1270, comprising the CDRL1 amino acid sequence of SEQ ID No: 1271 (IMGT), the CDRL2 amino acid sequence of SEQ ID No: 1272 (IMGT), and the CDRL3 amino acid sequence of SEQ ID No: 1273 (IMGT). The light chain nucleic acid sequence of the VL domain is SEQ ID No: 1269. The VH domain may be combined with any of the heavy chain constant region sequences described herein (the nucleotide and corresponding amino acid sequences of which are set out in Table 2). The VL domain may be combined with any ofthe light chain constant region sequences described herein (the nucleotide and corresponding amino acid sequences of which are set out in Table 2).

### CDRS, VL/VH:

In some examples the antibody comprises a variable heavy (VH) domain sequence comprising complementarity determining regions (CDRs) HCDR1, HCDR2 and HCDR3, and a variable light (VL) domain sequence comprising complementarity determining regions LCDR1, LCDR2 and LCDR3, wherein the HCDR3 is the HCDR3 of any one of the antibodies described herein and set out in Table 1 (Table 1a and/or Table 1b).

In some examples, the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3, wherein the CDRs are those of any one of the antibodies described herein and set out in Table 1 (Table 1a and/or Table 1b).

In some examples, the antibody comprises a variable heavy (VH) domain sequence and a variable light (VL) domain sequence and wherein the variable heavy (VH) domain and variable light (VL) domain sequences respectively comprise the variable heavy (VH) domain and variable light (VL) domain sequences of any one of the antibodies described herein and set out in Table 1 (Table 1a and/or Table 1b), optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

In some examples, the antibody comprises a variable heavy (VH) domain sequence and a variable light (VL) domain sequence and wherein the variable heavy (VH) domain and variable light (VL) domain sequences respectively comprise a sequence having at least 90% (preferably 95%, more preferably 98%) identity to the variable heavy (VH) and variable light (VL) domain sequences of any one of the antibodies described herein and set out in Table 1 (Table 1a and/or Table 1b), provided thatthe antibody has the CDRS of said antibody described herein and set out in Table 1 (Table 1a and/or Table 1b).

In work underlying the present invention, antibody sequences were recovered from antigen-binding B cells or from plasma cells from immunised mice as described elsewhere herein, and grouped into the clusters shown in FIGS. 3 and FIGS. 15 to 26 using bioinformatics analysis. It will be understood that antibodies in the same cluster (FIG. 3 and FIGS. 15 to 26) share a degree of sequence identity and/or conserved sequences. As such, antibodies in the same cluster might be considered as ‘sibling antibodies’. Sibling antibodies are within the scope of the present invention. In one embodiment, the present invention provides an expanded group of antibodies consisting of any antibody disclosed herein together with its sibling antibodies. In one embodiment, the present invention provides an expanded group of antibodies consisting of any group of antibodies disclosed herein together with their sibling antibodies. The present invention also provides antibodies having at least 90% (preferably 95%, more preferably 98%) identity to the variable heavy (VH) and variable light (VL) domain sequences of any one of the antibodies described herein and set out in Table 1 (Table 1a and/or Table 1b), provided that any substitutions in the VH and VL domain sequences are to amino acid residues present in a sibling antibody in the same cluster disclosed herein. The present invention also provides antibodies comprising a variable heavy (VH) domain sequence and a variable light (VL) domain sequence and wherein the variable heavy (VH) domain and variable light (VL) domain sequences respectively comprise the variable heavy (VH) domain and variable light (VL) domain sequences of any one of the antibodies described herein and set out in Table 1 (Table 1a and/or Table 1b), optionally with 1, 2, 3, 4 or 5 amino acid substitutions in the variable heavy (VH) domain sequence and optionally with 1, 2, 3, 4 or 5 amino acid substitutions in the variable light (VL) domain sequence, provided that any substitutions in the VH and VL domain sequences are to amino acid residues present in a sibling antibody in the same cluster disclosed herein.

One or more substitutions may be introduced in an antibody VH or VL domain at a position at which a different residue is present in a sibling antibody as shown in the clusters of FIGS. 3 and 15 to 26 herein. Thus, for example, an antibody may comprise the VH and VL domain of an IMPI antibody or YANG antibody disclosed herein, with one or more substitutions in framework regions, where those one or more substitutions are at positions shown to be variable in the cluster (optionally, at positions that vary between siblings obtained from antigen-binding B cells in the cluster and/or siblings for which assay data are presented herein). For cluster 1, for example, IMGT position 67 is variable since either Tyr or Asn may be present. Optionally, the substituted residue is the amino acid residue present in the sibling sequence (preferably, the sequence of a sibling obtained from an antigen-binding B cell or a sibling for which assay data are presented herein). Thus, a Y67N mutation may be introduced in a cluster 1 antibody, reflecting the residue present in IMPI-043. Conversely, N67Y mutation may be introduced in the VH domain of IMPI-043, reflecting the residue present in the other siblings of this cluster. As noted, siblings which were recovered from plasma cells (i.e., not recovered via antigen-binding of their expressing B cell) and for which assay data are not shown herein may optionally be discounted for this analysis. After subtracting such siblings from the clusters, the remaining siblings in each cluster are:
Cluster 1: IMPI-016, IMPI-024, IMPI-026, IMPI-034, IMPI-043, IMPI-050, IMPI-051, IMPI-058
Cluster 2: IMPI-001, IMPI-007, IMPI-019, IMPI-020, IMPI-023, IMPI-025, IMPI-030, IMPI-032, IMPI-039, IMPI-040, IMPI-053
Cluster 3: IMPI-014, IMPI-018, IMPI-027, IMPI-033, IMPI-045, IMPI-048
Cluster 4: IMPI-008, IMPI-010, IMPI-022, IMPI-035
Cluster 5: IMPI-005, IMPI-029, IMPI-056
Cluster 6: IMPI-002, IMPI-052
Cluster 7: IMPI-041, IMPI-055
Cluster 8: IMPI-003, IMPI-013
Cluster 9: IMPI-042, IMPI-054
Cluster 10: IMPI-021, IMPI-060
Cluster 11: IMPI-061, IMPI-062, IMPI-063, IMPI-064, IMPI-065, IMPI-066, IMPI-069, IMPI-070, IMPI-071
Cluster 12: IMPI-067
Cluster 13: YANG-1401, YANG-1401a, YANG-1401b, YANG-1401c, YANG-1401d, YANG-1401e
Cluster 14: YANG 2107, YANG-2108, YANG-2108a, YANG-2108b, YANG-2108c, YANG-2108d,
YANG-2108e, YANG-2108f, YANG-2108g, YANG-2108h, YANG-2108i, YANG-2108j, YANG-2108k, YANG-21081
Cluster 15: YANG-2111, YANG-2111a, YANG-2111b
Cluster 16: YANG-2203, YANG-2203a, YANG-2203b, YANG-2203c, YANG-2203d, YANG-2203e, YANG-2203f, YANG-2203g, YANG-2203h, YANG-2203i, YANG-2203j, YANG-2203k.
Cluster 17: YANG-2204, YANG-2205, YANG-2206, YANG-2207, YANG-2208, YANG-2209, YANG-2210, YANG-2211, YANG-2212, YANG-2213, YANG-2214, YANG-2215, YANG-2216, YANG-2217, YANG-2218, YANG-2219, YANG-2220, YANG-2221, YANG-2222, YANG-2223, YANG-2224, YANG-2225, YANG-2226, YANG-2227, YANG-2228, YANG-2229, YANG-2230, YANG-2231, YANG-2232, YANG-2233, YANG-2234, YANG-2235, YANG-2236, YANG-2237, YANG-2238, YANG-2239, YANG-2240, YANG-2241, YANG-2242, YANG-2243, YANG-2244, YANG-2245, YANG-2246, YANG-2247, YANG-2248, YANG-2249, YANG-2250, YANG-2251, YANG-2252, YANG-2253, YANG-2254, YANG-2255, YANG-2256, YANG-2257, YANG-2258, YANG-2259, YANG-2260, YANG-2261, YANG-2262, YANG-2263, YANG-2264, YANG-2265, YANG-2266, YANG-2267, YANG-2268, YANG-2269, YANG-2270, YANG-2271, YANG-2272, YANG-2273, YANG-2274, YANG-2275, YANG-2276, YANG-2277, YANG-2278, YANG-2279, YANG-2280, YANG-2281, YANG-2282, YANG-2283, YANG-2284, YANG-2285, YANG-2286, YANG-2287, YANG-2288, YANG-2289, YANG-2290, YANG-2291, YANG-2292, YANG-2293, YANG-2294, YANG-2295, YANG-2296, YANG-2297, YANG-2298, YANG-2299, YANG-2299a, YANG-2299b, YANG-2299c, YANG-2299d, YANG-2299e, YANG-2299f, YANG-2299g, YANG-2299h, YANG-2299i, YANG-2299j, YANG-2299k, YANG-22991
Cluster 18: YANG-1112, YANG-1112a, YANG-1112b, YANG-1112c

When one or more mutations (whether additions, insertions, substitutions or deletions of one or more amino acids) are made in the variable domain sequence of an antibody described herein, whether in a CDR or framework region, the resulting antibody may be tested (e.g., in one or more assays described herein) to confirm that affinity and/or potency are retained.

In some examples, the antibody comprises a variable heavy (VH) domain sequence and a variable light (VL) domain sequence and wherein the variable heavy (VH) domain and variable light (VL) domain sequences respectively comprise the variable heavy (VH) and variable light (VL) domain sequences of any one of the antibodies described herein and set out in Table 1 (Table 1a and/or Table 1b).

### Gene Segments:

In some aspects, the antibody comprises VH and/or VL domain and framework regions of human germline gene segment sequences. Gene segment sequences from which the exemplary antibodies described herein are derived are set out in Table 3.

In one example, the antibody comprises an antibody VH domain which is derived from recombination of a human heavy chain V gene segment, a human heavy chain D gene segment and a human heavy chain J gene segment.

In one example, the antibody comprises an antibody VH domain which is derived from recombination of a human heavy chain V gene segment, a human heavy chain D gene segment and a human heavy chain J gene segment, wherein the V gene segment is IGHV3-53*01, IGHV1-8*01 or IGHV3-33*01; and/or the J gene segment is IGHJ6*02, IGHJ4*02 or IGHJ3*02. In one example, the antibody comprises framework regions FR1, FR2, FR3 and FR4, wherein FR1 aligns with human germline V gene segment IGHV3-53*01, IGHV1-8*01 or IGHV3-33*01 with up to 1, 2, 3, 4, or 5 amino acid alterations, FR2 aligns with human germline V gene segment IGHV3-53*01, IGHV1-8*01 or IGHV3-33*01 with up to 1, 2, 3, 4, or 5 amino acid alterations, FR3 aligns with human germline V gene segment IGHV3-53*01, IGHV1-8*01 or IGHV3-33*01 with up to 1, 2, 3, 4 or 5 amino acid alterations, and/or FR4 aligns with human germline J gene segment IGHJ6*02, IGHJ4*02 or IGHJ3*02 with up to 1, 2, 3, 4 or 5 amino acid alterations.

In one example, the antibody comprises an antibody VH domain which is derived from recombination of a human heavy chain V gene segment, a human heavy chain D gene segment and a human heavy chain J gene segment, wherein the V gene segment is IGHV4-4*02, IGHV3-9*01 or IGHV3-30*18; and/or the J gene segment is IGHJ4*02 or IGHJ6*02. In one example, the antibody comprises framework regions FR1, FR2, FR3 and FR4, wherein FR1 aligns with human germline V gene segment IGHV4-4*02, IGHV3-9*01 or IGHV3-30*18 with up to 1, 2, 3, 4, or 5 amino acid alterations, FR2 aligns with human germline V gene segment IGHV4-4*02, IGHV3-9*01 or IGHV3-30*18 with up to 1, 2, 3, 4, or 5 amino acid alterations, FR3 aligns with human germline V gene segment IGHV4-4*02, IGHV3-9*01 or IGHV3-30*18 with up to 1, 2, 3, 4 or 5 amino acid alterations, and/or FR4 aligns with human germline J gene segment IGHJ4*02 or IGHJ6*02 with up to 1, 2, 3, 4 or 5 amino acid alterations.

In one example, the antibody comprises an antibody VH domain which is derived from recombination of a human heavy chain V gene segment, a human heavy chain D gene segment and a human heavy chain J gene segment, wherein the V gene segment is IGHV3-9*01 or IGHV3-20*d01; and/or the J gene segment is IGHJ6*02 or IGHJ4*02. In one example, the antibody comprises framework regions FR1, FR2, FR3 and FR4, wherein FR1 aligns with human germline V gene segment IGHV3-9*01 or IGHV3-20*d01 with up to 1, 2, 3, 4, or 5 amino acid alterations, FR2 aligns with human germline V gene segment IGHV3-9*01 or IGHV3-20*d01 with up to 1, 2, 3, 4, or 5 amino acid alterations, FR3 aligns with human germline V gene segment IGHV3-9*01 or IGHV3-20*d01 with up to 1, 2, 3, 4 or 5 amino acid alterations, and/or FR4 aligns with human germline J gene segment IGHJ6*02 or IGHJ4*02 with up to 1, 2, 3, 4 or 5 amino acid alterations.

In one example, the antibody comprises an antibody VH domain which is derived from recombination of a human heavy chain V gene segment, a human heavy chain D gene segment and a human heavy chain J gene segment, wherein the V gene segment is IGHV5-51*01, IGHV4-31*03, IGHV3-53*01 or IGHV3-30*18; and/or the J gene segment is IGHJ4*02 or IGHJ6*02. In one example, the antibody comprises framework regions FR1, FR2, FR3 and FR4, wherein FR1 aligns with human germline V gene segment IGHV5-51*01, IGHV4-31*03, IGHV3-53*01 or IGHV3-30*18 with up to 1, 2, 3, 4, or 5 amino acid alterations, FR2 aligns with human germline V gene segment IGHV5-51*01, IGHV4-31*03, IGHV3-53*01 or IGHV3-30*18 with up to 1, 2, 3, 4, or 5 amino acid alterations, FR3 aligns with human germline V gene segment IGHV5-51*01, IGHV4-31*03, IGHV3-53*01 or IGHV3-30*18 with up to 1, 2, 3, 4 or 5 amino acid alterations, and/or FR4 aligns with human germline J gene segment IGHJ4*02 or IGHJ6*02 with up to 1, 2, 3, 4 or 5 amino acid alterations.

In one example, the antibody comprises an antibody VL domain which is derived from recombination of a human light chain V gene segment and a human light chain J gene segment.

In one example, the antibody comprises an antibody VL domain which is derived from recombination of a human light chain V gene segment and a human light chain J gene segment, wherein the V gene segment is IGKV1-9*d01, IGKV6-21*01, IGKV1-33*01 orIGKV3-20*01, and/or the Jgene segment is IGKJ5*01, IGKJ4*01, IGKJ3*01, IGKJ2*04 or IGKJ1*01. In one example, the antibody comprises framework regions FR1, FR2, FR3 and FR4, wherein FR1 aligns with human germline V gene segment IGKV1-9*d01, IGKV6-21*01, IGKV1-33*01 or IGKV3-20*01 with up to 1, 2, 3, 4, or 5 amino acid alterations, FR2 aligns with human germline V gene segment IGKV1-9*d01, IGKV6-21*01, IGKV1-33*01 or IGKV3-20*01 with up to 1, 2, 3, 4, or 5 amino acid alterations, FR3 aligns with human germline V gene segment IGKV1-9*d01, IGKV6-21*01, IGKV1-33*01 or IGKV3-20*01 with up to 1, 2, 3, 4 or 5 amino acid alterations, and/or FR4 aligns with human germline J gene segment IGKJ5*01, IGKJ4*01, IGKJ3*01, IGKJ2*04 or IGKJ1*01 with up to 1, 2, 3, 4 or 5 amino acid alterations.

In one example, the antibody comprises an antibody VL domain which is derived from recombination of a human light chain V gene segment and a human light chain J gene segment, wherein the V gene segment is IGKV2D-30*01, IGKV1D-13*d01 or IGKV3-20*01, and/or the J gene segment is IGKJ4*01. In one example, the antibody comprises framework regions FR1, FR2, FR3 and FR4, wherein FR1 aligns with human germline V gene segment IGKV2D-30*01, IGKV1D-13*d01 or IGKV3-20*01 with up to 1, 2, 3, 4, or 5 amino acid alterations, FR2 aligns with human germline V gene segment IGKV2D-30*01, IGKV1D-13*d01 or IGKV3-20*01 with up to 1, 2, 3, 4, or 5 amino acid alterations, FR3 aligns with human germline V gene segment IGKV2D-30*01, IGKV1D-13*d01 or IGKV3-20*01 with up to 1, 2, 3, 4 or 5 amino acid alterations, and/or FR4 aligns with human germline J gene segment IGKJ4*01 with up to 1, 2, 3, 4 or 5 amino acid alterations.

In one example, the antibody comprises an antibody VL domain which is derived from recombination of a human light chain V gene segment and a human light chain J gene segment, wherein the V gene segment is IGKV1-6*01 or IGKV3-20*01, and/or the J gene segment is IGKJ1*01 or IGKJ2*04. In one example, the antibody comprises framework regions FR1, FR2, FR3 and FR4, wherein FR1 aligns with human germline V gene segment IGKV1-6*01 or IGKV3-20*01 with up to 1, 2, 3, 4, or 5 amino acid alterations, FR2 aligns with human germline V gene segment IGKV1-6*01 or IGKV3-20*01 with up to 1, 2, 3, 4, or 5 amino acid alterations, FR3 aligns with human germline V gene segment IGKV1-6*01 or IGKV3-20*01 with up to 1, 2, 3, 4 or 5 amino acid alterations, and/or FR4 aligns with human germline J gene segment IGKJ1*01 or IGKJ2*04 with up to 1, 2, 3, 4 or 5 amino acid alterations.

In one example, the antibody comprises an antibody VL domain which is derived from recombination of a human light chain V gene segment and a human light chain J gene segment, wherein the V gene segment is IGKV1D-13*d01, IGKV3-20*01 or IGKV1-12*01, and/or the J gene segment is IGKJ1*01, IGKJ4*01 or IGKJ3*01. In one example, the antibody comprises framework regions FR1, FR2, FR3 and FR4, wherein FR1 aligns with human germline V gene segment IGKV1D-13*d01, IGKV3-20*01 or IGKV1-12*01 with up to 1, 2, 3, 4, or 5 amino acid alterations, FR2 aligns with human germline V gene segment IGKV1D-13*d01, IGKV3-20*01 or IGKV1-12*01 with up to 1, 2, 3, 4, or 5 amino acid alterations, FR3 aligns with human germline V gene segment IGKV1D-13*d01, IGKV3-20*01 or IGKV1-12*01 with up to 1, 2, 3, 4 or 5 amino acid alterations, and/or FR4 aligns with human germline J gene segment IGKJ1*01, IGKJ4*01 or IGKJ3*01 with up to 1, 2, 3, 4 or 5 amino acid alterations.

### Antibody Properties:

In some examples, the antibody is a monoclonal antibody. Methods of making monoclonal antibodies are known and include, for example, fusing myeloma cells with the cells from an animal that was immunized with the desired antigen. In other examples, the monoclonal antibodies may be generated using recombinant DNA technology. In one example, the antibody is a monoclonal antibody that specifically binds the SARS-CoV-2 spike protein. In one example, the antibody is a fully human monoclonal antibody.

In some examples, the antibody is a human antibody. In one example, the antibody is a fully human antibody. In one example, the antibody is a fully human monoclonal antibody.

### Sequence Identity:

In some examples, the antibody comprises an amino acid sequence which has a high level of sequence identity to the amino acid sequence of one of the exemplary antibodies described herein and set out in Table 1 (Table 1a and/or Table 1b).

In one example, the amino acid sequence is at least 70% identical to the specified SEQ ID No. In one example, the amino acid sequence is at least 75% identical to the specified SEQ ID No. In one example, the amino acid sequence is at least 95% identical to the specified SEQ ID No. In one example, the amino acid sequence is at least 96% identical to the specified SEQ ID No. In one example, the amino acid sequence is at least 97% identical to the specified SEQ ID No. In one example, the amino acid sequence is at least 98% identical to the specified SEQ ID No. In one example, the amino acid sequence is at least 99% identical to the specified SEQ ID No. In one example, the amino acid sequence is at least 99.5% identical to the specified SEQ ID No.

### Substitutions:

In some examples, the antibody comprises amino acid substitutions.

Amino acid substitutions include alterations in which an amino acid is replaced with a different naturally-occurring amino acid residue. Such substitutions may be classified as “conservative”, in which case an amino acid residue contained in a polypeptide is replaced with another naturally occurring amino acid of similar character either in relation to polarity, side chain functionality or size. Such conservative substitutions are well known in the art. Substitutions encompassed by the present invention may also be “non-conservative”, in which an amino acid residue which is present in a peptide is substituted with an amino acid having different properties, such as naturally-occurring amino acid from a different group (e.g. substituting a charged or hydrophobic amino; acid with alanine), or alternatively, in which a naturally-occurring amino acid is substituted with a non-conventional amino acid.

In one embodiment, the conservative amino acid substitutions are as described herein. For example, the substitution may be of Y with F, T with S or K, P with A, E with D or Q, N with D or G, R with K, G with N or A, T with S or K, D with N or E, I with L or V, F with Y, S with T or A, R with K, G with N or A, K with R, A with S, K or P. In another embodiment, the conservative amino acid substitutions may be wherein Y is substituted with F, T with A or S, I with L or V, W with Y, M with L, N with D, G with A, T with A or S, D with N, I with L or V, F with Y or L, S with A or T and A with S, G, T or V.

In one example, the amino acid substitutions are located outside the CDR sequences.

### Light Chains:

In some examples, the antibody comprises a kappa light chain. Kappa light chain constant region amino acid and nucleotide sequences are set out in SEQ ID Nos: 447-456.

In one example, the light chain may be a lambda light chain. Lambda light chain constant region amino acid and nucleotide sequences can be found in SEQ ID Nos: 457-481.

### Isotypes, Constant Regions+Modifications:

In some examples, the antibodies may block the progress of an infection at the point of viral entry into a cell, by binding to the virus and preventing it infecting the cell (neutralisation). The antibody may then further mediate uptake and destruction of the virus by immune cells (opsonisation). The antibody may further mediate the disruption of the virus lipid envelope by the fixation of complement. In other examples, the antibodies facilitate the killing of an infected cell via antibody dependent cellular cytotoxicity (ADCC), where antibodies bind to infected cells and allow immune cells to kill them. Selection of an appropriate format for the antibody (e.g., IgG4 or IgG1), can be used to achieve one or more of these outcomes.

Infection can in principle be prevented by high potency neutralising antibodies that bind with high affinity to the virus spike protein and prevent cell entry. The format for that antibody could be an antibody with limited Fc effector functions, e.g., an IgG4, e.g., a stabilised IgG4 isotype. Administration of an anti-IgG4 antibody in a prophylactic setting should give protective viral neutralising activity, however, repeated doses of the antibody may be required every 3-4 weeks to maintain protective efficacy and until the risk of infection has reduced. In a therapeutic setting such an antibody would also neutralise virus and reduce virus load thereby possibly impacting on disease severity.

The same potent spike binding antibody can alternatively be formatted to neutralise virus entry and target infected cells for killing by inclusion of a portion with Fc effector function, e.g., an IgG1 constant region.

An effector enabled antibody may recruit natural killer cells to infected cells to achieve ADCC, facilitate opsonisation of virus particles to allow engulfment and destruction by macrophages and/or target virus particles for complement deposition.

The antibodies described herein may comprise a constant region, such as a human constant region, for example an effector-null human constant region, e.g. an IgG4 constant region or an IgG1 constant region, optionally wherein the constant region is IgG4-PE (SEQ ID Nos: 441-446 and 482-483), or a disabled IgG1 as defined in SEQ ID Nos: 425-426.

In other embodiments, the antibody is any of the isotypes or constant regions as defined hereinabove. In one embodiment, the constant region is wild-type human IgG1 (SEQ ID Nos: 417-424). For example, the constant region is an effector-enabled IgG1 constant region, optionally having ADCC and/or CDC activity.

In one embodiment, the constant region is engineered for enhanced ADCC and/or CDC and/or ADCP. In another embodiment, the constant region is engineered for enhanced effector function.

In some embodiments, the antibody may comprise modifications that enhance the ability ofthe antibody to cluster and therefore be a better substrate for complement fixation. The Fc domain of IgG1 may be mutated for example at E345 or E430 to reinforce inter-antibody Fc:Fc interactions, stimulating formation of hexamers, which enhances the induction of CDC and ADCC of target cells (de Jong et al., PloS Biol 14(1) e1002344 2016). Hexamer formation is optionally combined with a bispecific antibody format.

The IgG4 constant region may be any of the IgG4 constant region amino acid sequences, or encoded by any of the nucleic acid sequences (SEQ ID Nos: 435-440). A heavy chain constant region may be an IgG4 comprising both the Leu235Glu mutation and the Ser228Pro mutation. This “IgG4-PE” heavy chain constant region (SEQ ID Nos: 441-446 and 482-483) is effector null.

An alternative effector null human constant region is a disabled IgG1 being an IgG1*01 allele comprising the L235A and/or G237A mutations (e.g. LAGA, SEQ ID Nos: 425-426). In one example, the antibodies or antibody fragments disclosed herein comprise an IgG1 heavy chain constant region, wherein the sequence contains alanine at position 235 and/or 237 (EU index numbering). The antibody-dependent cell phagocytosis (ADCP) mechanism is discussed in Gil et al., “Antibody-Dependent Phagocytosis of Tumor Cells by Macrophages: A Potent Effector Mechanism of Monoclonal Antibody Therapy of Cancer”, Cancer Res., 75(23), Dec. 1, 2015.

The potency of Fc-mediated effects may be enhanced by engineering the Fc domain by various established techniques. Such methods increase the affinity for certain Fc-receptors or decrease the affinity for inhibitory Fc-receptors, thus creating potential diverse profiles of activation enhancement. This can be achieved by modification of one or several amino acid residues (e.g. as described in Lazar et al., 2006, Proc. Natl. Acad. Sci. U.S.A., Mar 14; 103(11):4005-10; the modifications disclosed therein are incorporated herein by reference). Human IgG1 constant regions containing specific mutations or altered glycosylation on residue Asn297 (e.g. N297Q, EU index numbering) have been shown to enhance binding to certain Fc receptors.

In one example, such mutations are one or more of the residues selected from 239, 332 and 330 for human IgG1 constant regions (or the equivalent positions in other IgG isotypes). In one example, the antibody or fragment comprises a human IgG1 constant region having one or more mutations independently selected from N297Q, S239D, 1332E and A330L (EU index numbering).

In another example, the increase in affinity for Fc-receptors is achieved by altering the natural glycosylation profile of the Fc domain by, for example, generating under fucosylated or de-fucosylated variants (as described in Natsume et al., 2009, Drug Des. Devel. Ther., 3:7-16 or by Zhou Q., Biotechnol. Bioeng., 2008, Feb 15, 99(3):652-65, the modifications described therein are incorporated herein by reference). Non-fucosylated antibodies harbour a tri-mannosyl core structure of complex-type N-glycans of Fc without fucose residue. These glycoengineered antibodies that lack core fucose residue from the Fc N-glycans may exhibit stronger ADCC than fucosylated equivalents due to enhancement of FcγRIIIa binding capacity. For example, to increase ADCC, residues in the hinge region can be altered to increase binding to Fc-yRIII (see, for example, Shields et al., 2001, J. Biol. Chem., Mar 2; 276(9):6591-604; the modifications described therein are incorporated herein by reference). Thus, in one example, the antibody or fragment comprises a human IgG heavy chain constant region that is a variant of a wild-type human IgG heavy chain constant region, wherein the variant human IgG heavy chain constant region binds to human Fcγ receptors selected from the group consisting of FcγRIIB and FcγRIIA with higher affinity than the wild type human IgG heavy chain constant region binds to the human Fcγ receptors. In one example, the antibody or fragment comprises a human IgG heavy chain constant region that is a variant of a wild type human IgG heavy chain constant region, wherein the variant human IgG heavy chain constant region binds to human FcγRIIB with higher affinity than the wild type human IgG heavy chain constant region binds to human FcγRIIB. In one example, the variant human IgG heavy chain constant region is a variant human IgG1, a variant human IgG2, or a variant human IgG4 heavy chain constant region. In one example, the variant human IgG heavy chain constant region comprises one or more amino acid mutations selected from G236D, P238D, S239D, S267E, L328F, and L328E (EU index numbering system). In another example the variant human IgG heavy chain constant region comprises a set of amino acid mutations selected from the group consisting of: S267E and L328F; P238D and L328E; P238D and one or more substitutions selected from the group consisting of E233D, G237D, H268D, P271G, and A330R; P238D, E233D, G237D, H268D, P271G, and A330R; G236D and S267E; S239D and S267E; V262E, S267E, and L328F; and V264E, S267E, and L328F (EU index numbering system). In another example, the variant human IgG heavy chain constant region further comprises one or more amino acid mutations that reduce the affinity of the IgG for human FcγRIIIA, human FcγRIIA, or human FcγRI. In one example, the FcγRIIB is expressed on a cell selected from the group consisting of macrophages, monocytes, B-cells, dendritic cells, endothelial cells, and activated T-cells. In one embodiment, the variant human IgG heavy chain constant region comprises one or more of the following amino acid mutations G236A, S239D, F243L, T256A, K290A, R292P, S298A, Y300L, V305I, A330L, 1332E, E333A, K334A, A339T, and P396L (EU index numbering system). In one example, the variant human IgG heavy chain constant region comprises a set of amino acid mutations selected from the group consisting of: S239D; T256A; K290A; S298A; 1332E; E333A; K334A; A339T; S239D and 1332E; S239D, A330L, and 1332E; S298A, E333A, and K334A; G236A, S239D, and 1332E; and F243L, R292P, Y300L, V305I, and P396L (EU index numbering system). In one example, the variant human IgG heavy chain constant region comprises a S239D, A330L, or 1332E amino acid mutations (EU index numbering system). In one example, the variant human IgG heavy chain constant region comprises an S239D and 1332E amino acid mutations (EU index numbering system). In one example, the variant human IgG heavy chain constant region is a variant human IgG1 heavy chain constant region comprising the S239D and 1332E amino acid mutations (EU index numbering system). In one example, the antibody or fragment comprises an afucosylated Fc region. In another example, the antibody or fragment thereof is defucosylated.

In another example, the antibody or fragment is under fucosylated.

In another example, the antibodies and fragments disclosed herein may comprise a triple mutation (M252Y/S254T/T256E) which enhances binding to FcRn. See Dall'Aqua et al., Immunol 2002; 169:5171-5180 for a discussion of mutations affection FcRn binding in table 2, the mutations described therein are incorporated herein by reference.

Equally, the enhancement of CDC may be achieved by amino acid changes that increase affinity for Clq, the first component of the classic complement activation cascade (see Idusogie et al., J. Immunol., 2001, 166:2571-2575; the modifications described are incorporated herein by reference). Another approach is to create a chimeric Fc domain created from human IgG1 and human IgG3 segments that exploit the higher affinity if IgG3 for Clq (Natsume et al., 2008, Cancer Res., 68: 3863-3872; the modifications are incorporated herein by reference). In another example, the antibody or antibody fragments disclosed herein may comprise mutated amino acids at residues 329, 331 and/or 322 to alter the Clq binding and/or reduced or abolished CDC activity. In another example, the antibodies or antibody fragments disclosed herein may contain Fc regions with modifications at residues 231 and 239, whereby the amino acids are replaced to alter the ability of the antibody to fix complement. In one example, the antibody or fragment has a constant region comprising one or more mutations selected from E345K, E430G, R344D and D356R, in particular a double mutation comprising R344D and D356R (EU index numbering system).

An antibody may have a heavy chain constant region that binds one or more types of Fc receptor but does not induce cellular effector functions, i.e. which does not mediate ADCC, CDC or ADCP activity. Such a constant region may be unable to bind the particular Fc receptor(s) responsible for triggering ADCC, CDC or ADCP activity. An antibody may have a heavy chain constant region that does not bind Fcγ receptors. Thus, in one example, the constant region may comprise a Leu235Glu mutation (EU index numbering system).

In another example, the antibodies disclosed herein are modified to increase or decrease serum half-life. In one embodiment, one or more of the following mutations: T252L, T254S or T256F are introduced to increase biological half-life of the antibody. Biological half-life can also be increased by altering the heavy chain constant region CH1 domain or CL region to contain a salvage receptor binding epitope taken from two loops of a CH2 domain of an Fc region of an IgG, as described in U.S. Pat. Nos. 5,869,046 and 6,121,022, the modifications described therein are incorporated herein by reference. In another example, the Fc hinge region of an antibody or antigen-binding fragment of the invention is mutated to decrease the biological half-life of the antibody or fragment. One or more amino acid mutations are introduced into the CH2-CH3 domain interface region of the Fc-hinge fragment such that the antibody or fragment has impaired Staphylococcyl protein A (SpA) binding relative to native Fc-hinge domain SpA binding. Other methods of increasing serum half-life are known to those skilled in the art. Thus, in one example, the antibody or fragment is PEGylated. In another example, the antibody or fragment is fused to an albumin-binding domain, e.g. an albumin binding single domain antibody (dAb). In another example, the antibody or fragment is PASylated (i.e. genetic fusion of polypeptide sequences composed of PAS (XL-Protein GmbH) which forms uncharged random coil structures with large hydrodynamic volume). In another example, the antibody or fragment is XTENylated®/rPEGylated (i.e. genetic fusion of non-exact repeat peptide sequence (Amunix, Versartis) to the therapeutic peptide). In another example, the antibody or fragment is ELPylated (i.e. genetic fusion to ELP repeat sequence (PhaseBio)). These various half-life extending fusions are described in more detail in Strohl, BioDrugs (2015) 29:215-239, which fusions are incorporated herein by reference.

The antibody may have a modified constant region which increases stability. Thus, in one example, the heavy chain constant region comprises a Ser228Pro mutation. In another example, the antibodies and fragments disclosed herein comprise a heavy chain hinge region that has been modified to alter the number of cysteine residues. This modification can be used to facilitate assembly of the light and heavy chains or to increase or decrease the stability of the antibody.

### Nucleic Acids, Vectors, Host Cells

Nucleic acids that encode a VH domain and/or a VL domain of any one of the antibodies described herein are also provided. The nucleic acid sequences encoding each of the VH and VL domains of each the exemplary antibodies described herein are set out in Tables 1a and 1b.

In one example, the nucleic acid sequence is at least 70% identical to the specified SEQ ID NO. In one example, the nucleic acid sequence is at least 80% identical to the specified SEQ ID NO. In one example, the nucleic acid sequence is at least 90% identical to the specified SEQ ID NO. In one example, the nucleic acid sequence is at least 95% identical to the specified SEQ ID NO. In one example, the nucleic acid sequence is at least 96% identical to the specified SEQ ID NO. In one example, the nucleic acid sequence is at least 97% identical to the specified SEQ ID NO. In one example, the nucleic acid sequence is at least 98% identical to the specified SEQ ID NO. In one example, the nucleic acid sequence is at least 99% identical to the specified SEQ ID NO. In one example, the nucleic acid sequence is at least 99.5% identical to the specified SEQ ID NO.

In one example, the nucleic acid encodes a heavy chain of any one of the antibodies described herein. In another example, the nucleic acid encodes a light chain of any one of the antibodies described herein.

In one example, the nucleic acid is an isolated and purified nucleic acid.

Vectors comprising the nucleic acids described above are also provided. In one example, the vector may be a CHO vector. In one example, the vector may be a HEK293 vector.

Host cells comprising the nucleic acids described above are also provided. In some examples, the host cells are eukaryotic cells, e.g., mammalian cells, preferably CHO cells (e.g., CHO cells grown in suspension culture).

### Pharmaceutical Composition

In one example, there is provided a pharmaceutical composition comprising an effective amount of an antibody as described herein and a pharmaceutically acceptable excipient. An effective amount of antibody to be employed therapeutically will depend, for example, upon the therapeutic objectives, the route of administration, and the condition of the patient. In one example, the composition includes other excipients or stabilizers.

Pharmaceutically acceptable excipients are known and include carriers, excipients, or stabilizers that are nontoxic to the cell or mammal being exposed thereto at the dosages and concentrations employed. Often the physiologically acceptable excipient is an aqueous pH buffered solution. Examples of physiologically acceptable excipient include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as Ethylenediaminetetraacetic acid (EDTA); sugar alcohols such as mannitol or sorbitol; salt-forming counterions such as sodium; and/or nonionic surfactants such as TWEEN™, polyethylene glycol (PEG), and PLURONICS™

The antibodies can be administered intravenously or through the nose, lung, for example, as a liquid or powder aerosol (lyophilized) or by nebulisation of a liquid. The composition can also be administered parenterally or subcutaneously. When administered systemically, the composition should be sterile, pyrogen-free and in a physiologically acceptable solution having due regard for pH, isotonicity and stability. These conditions are known to those skilled in the art.

Methods of administering a prophylactic or therapeutic agent (e.g., an antibody as disclosed herein), or pharmaceutical composition include, but are not limited to, parenteral administration (e.g., intradermal, intramuscular, intraperitoneal, intravenous and subcutaneous), epidural, and mucosal (e.g., intranasal and oral routes). In a specific example, a prophylactic or therapeutic agent (e.g., an antibody as disclosed herein), or a pharmaceutical composition is administered intranasally, intramuscularly, intravenously, or subcutaneously. The prophylactic or therapeutic agents, or compositions may be administered by any convenient route, for example by infusion or bolus injection, by absorption through epithelial or mucocutaneous linings (e.g., oral mucosa, intranasal mucosa, rectal and intestinal mucosa, etc.) and may be administered together with other biologically active agents. Administration can be systemic or local. Each dose may or may not be administered by an identical route of administration. In one example, an anti-SARS-CoV-2 antibody as disclosed herein may be administered via multiple routes of administration simultaneously or subsequently to other doses of the same or a different anti-SARS-CoV-2 antibody as disclosed herein.

Various delivery systems are known and can be used to administer a prophylactic or therapeutic agent (e.g., an antibody as disclosed herein), including, but not limited to, encapsulation in liposomes, microparticles, microcapsules, recombinant cells capable of expressing the antibody, receptor-mediated endocytosis (see, e.g., Wu and Wu, J. Biol. Chem. 262:4429-4432 (1987)), construction of a nucleic acid as part of a retroviral or other vector, etc. In addition, pulmonary administration can also be employed, e.g., by use of an inhaler or nebulizer, and formulation with an aerosolizing agent. See, e.g., U.S. Pat. Nos. 6,019,968, 5,985,320, 5,985,309, 5,934,272, 5,874,064, 5,855,913, 5,290,540, and 4,880,078; and PCT Publication Nos. WO92/19244, WO97/32572, WO97/44013, WO98/31346, and WO99/66903, each of which is incorporated herein by reference their entirety.

In a specific example, it may be desirable to administer a prophylactic or therapeutic agent, or a pharmaceutical composition as described herein locally to the area in need of treatment. This may be achieved by, for example, local infusion, by topical administration (e.g., by intranasal spray), by injection, or by means of an implant, said implant being of a porous, non-porous, or gelatinous material, including membranes, such as sialastic membranes, or fibres. When administering an anti-SARS-CoV-2 antibody, care must be taken to use materials to which the antibody does not absorb.

In the case of medicaments that are intended for local and/or topical administration, such as by absorption to epithelial or mucocutaneous linings, an antibody may be provided as an IgA isotype antibody. For human patients, human IgA1 or human IgA2 antibodies are preferred. Medicaments formulated for inhalation and/or for delivery of antibody (or its encoding nucleic acid, e.g., in a DNA vector) to the upper and/or lower respiratory tract, including formulations for delivery of a nebulised medicament, may comprise an IgA (e.g., human IgA1 or human IgA2) antibody. Inhalers, nebulisers and similar devices may thus be provided containing a medicament comprising an IgA antibody or its encoding nucleic acid, together with any buffers or other excipients suitable for stabilisation of the medicament and/or for promoting its delivery to the target tissue.

### Therapeutic Use

Antibodies described herein may be used to treat or prevent a SARS-CoV-2-related disease or condition, such as COVID-19. One aspect includes use of an antibody or composition described herein as a medicament.

Thus, in one example antibodies described herein or compositions described herein for use in a method of treating a SARS-CoV-2-related disease or condition are provided, said method comprising administering the antibody or composition to a patient. In another example, antibodies described herein or compositions described herein for use in a method of preventing a SARS-CoV-2-related disease or condition are provided, said method comprising administering the antibody or composition to a patient.

In one example, the SARS-CoV-2-related disease or condition is a SARS-CoV-2-mediated disease or condition.

Preferably, the SARS-CoV-2-related disease or condition is a COVID-19-related disease or condition. In some examples, the COVID-19-related disease or condition is COVID-19. In some examples, the COVID-19-related disease or condition is long manifestation of infection by SARS-CoV-2 such as ‘Long COVID’.

In one example, the antibody for use or the composition for use described above, one or more symptoms of COVID-19 are reduced. In one example, the progression of SARS-CoV-2 infection is reduced. In one example, the risk of developing COVID-19 is reduced. In one example, the risk of transmission of SARS-CoV-2 to and/or from a human is reduced.

In one example, said method further comprises administering at least one further therapeutic agent. In one example, the first antibody and further therapeutic agent are administered simultaneously, separately, or sequentially.

In one example, the further therapeutic agent is a further antibody. Accordingly, monoclonal antibodies of the invention might be administered as part of an antibody cocktail comprising multiple (e.g., 2, 3 or 4) different monoclonal antibodies.

The further antibody may be selected from:
i. an antibody that specifically binds to the receptor binding domain (RBD) of the S1 subunit of the SARS-CoV-2 spike protein and competes for binding to the SARS-CoV-2 spike protein with the human ACE2 receptor;
ii. an antibody that specifically binds to the receptor binding domain (RBD) of the S1 subunit of the SARS-CoV-2 spike protein and does not compete for binding to the SARS-CoV-2 spike protein with the human ACE2 receptor;
iii. an antibody that specifically binds to the N-terminal domain (NTD) of the S1 subunit of the of SARS-CoV-2 spike protein;
iv. an antibody that specifically binds to the S2 subunit of the of SARS-CoV-2 spike protein; and v. an antibody preferentially binds to the trimer form of the SARS-CoV-2 spike protein over the isolated RBD domain, S1 subunit and S2 subunit of the SARS-CoV-2 spike protein.

The further antibody might bind to the same or a different subunit of SARS-CoV-2 as the first antibody.

The further antibody might bind to the same or a different domain of SARS-CoV-2 as the first antibody.

An antibody cocktail might comprise a first antibody and a second antibody and optionally one or more further antibodies.

In one example, where the first antibody binds to the RBD of the SARS-CoV-2 spike protein and competes for binding to the SARS-CoV-2 spike protein with the human ACE2 receptor, the second antibody also specifically binds to the receptor binding domain (RBD) of the S1 subunit of the of SARS-CoV-2 spike protein.

An antibody cocktail might comprise, for example:
i. a first antibody that specifically binds to the receptor binding domain (RBD) of the S1 subunit of SARS-CoV-2 and which competes with ACE2 for binding to SARS-CoV-2; and
ii. a second antibody that also specifically binds to the receptor binding domain (RBD) of the S1 subunit of SARS-CoV-2 and which competes with ACE2 for binding to SARS-CoV-2.

An antibody cocktail might comprise, for example:
iii. a first antibody that specifically binds to the receptor binding domain (RBD) of the S1 subunit of SARS-CoV-2 and which competes with ACE2 for binding to SARS-CoV-2; and
iv. a second antibody that specifically binds to the receptor binding domain (RBD) of the S1 subunit of SARS-CoV-2 outside the ACE2 epitope region, such that the second antibody does not compete with ACE2 for binding to SARS-CoV-2.

Alternatively, in one example, where the first antibody binds to the RBD of the SARS-CoV-2 spike protein and competes for binding to the SARS-CoV-2 spike protein with the human ACE2 receptor, the second antibody specifically binds to the N-terminal domain (NTD) of the S1 subunit of the of SARS-CoV-2 spike protein. In another example, where the first antibody binds to the RBD of the SARS-CoV-2 spike protein and competes for binding to the SARS-CoV-2 spike protein with the human ACE2 receptor, the second antibody the further antibody specifically binds to the S2 subunit of the of SARS-CoV-2 spike protein. In still another example, where the first antibody binds to the RBD of the SARS-CoV-2 spike protein and competes for binding to the SARS-CoV-2 spike protein with the human ACE2 receptor, the second antibody preferentially binds to the trimer form of the SARS-CoV-2 spike protein over the isolated RBD domain, S1 subunit and S2 subunit of the SARS-CoV-2 spike protein

Provided herein is the use of an antibody described herein or a composition described herein in the manufacture of a medicament for treating a SARS-CoV-2-related disease or condition. Use of an antibody described herein or a composition described herein in the manufacture of a medicament for preventing a SARS-CoV-2- related disease or condition is also provided. Preferably, the SARS-CoV-2- related disease or condition is COVID-19. Thus, in one example, one or more symptoms of COVID-19 are reduced. In another example, the progression of SARS-CoV-2 infection is reduced. In another example, the risk of developing COVID-19 is reduced. In another example, the risk of transmission of SARS-CoV-2 to and/or from a human is reduced.

In one example, the use of an antibody or composition described herein further comprises administering at least one further therapeutic agent. In one example, the first antibody and further therapeutic agent are administered simultaneously, separately or sequentially. In one example, the further therapeutic agent is a further antibody as defined herein.

A method of treating a SARS-CoV-2-related disease or condition in a human, comprising administering to said human a therapeutically effective amount of an antibody described herein or a composition described herein is provided. A method of preventing a SARS-CoV-2-related disease or condition in a human, comprising administering to said human a therapeutically effective amount of an antibody described herein or a composition described herein is also provided. Preferably, the SARS-CoV-2-related disease or condition is COVID-19. In one example, one or more symptoms of COVID-19 are reduced. In one example, the progression of SARS-CoV-2 infection is reduced. In one example, the risk of developing COVID-19 is reduced. In one example, the risk of transmission of SARS-CoV-2 to and/or from a human is reduced.

In one example, said method further comprising administering at least one further therapeutic agent. In one example, the first antibody and further therapeutic agent are administered simultaneously, separately or sequentially. In one example, the further therapeutic agent is a further antibody as defined anywhere herein.

In one example, the antibody is administered as an antibody-drug conjugate in which the antibody is linked to a drug moiety. For example, the antibody may be linked to a drug moiety which may be a cytokine, chemokine, or small molecule antiviral.

Antibodies described herein may be used to prevent death and shorten the time to recovery and discharge for COVID19 patients. Patients will generally be human patients and may be patients admitted to hospital and diagnosed as testing positive for SARS-CoV-2 and/or suspected or believed to be suffering from COVID19. Patient groups for treatment include all people hospitalised with COVID-19.

In some examples, antibodies described herein or pharmaceutical compositions comprising the antibody as described herein may be used singly or in combination with other anti-SARS-CoV-2 antibodies or pharmaceutical compositions comprising other anti-SARS-CoV-2 antibodies. Combinations of two or more anti-SARS-CoV-2 antibodies may have additive or synergistic potency compared to the potency of a single antibody, e.g. as measured in a pseudovirus assay or by the live virus assay. In some examples, combinations of RBD and S2 antibodies have additive potency over a single mAb. In some examples, combinations of RBD antibodies have additive potency over a single mAb. In some examples, combinations of S2 antibodies have additive potency over a single mAb. In some examples, combinations of RBD and NTD antibodies have additive potency over a single mAb. In some examples, combinations of S2 and NTD antibodies have additive potency over a single mAb. In some examples, combinations of RBD and S2 antibodies have synergistic potency over a single mAb. In some examples, combinations of RBD antibodies have synergistic potency over a single mAb. In some examples, combinations of S2 antibodies have synergistic potency over a single mAb. In some examples, combinations of RBD and NTD antibodies have synergistic potency over a single mAb. In some examples, combinations of S2 and NTD antibodies have synergistic potency over a single mAb. In some examples, triple combinations of RBD, S2 and NTD antibodies have additive potency over a single mAb. In some examples, triple combinations of RBD, S2 and NTD antibodies have synergistic potency over a single mAb.

In some examples, the antibody as described herein or pharmaceutical composition comprising the antibody as described herein is administered in combination with a directly acting antiviral (DAA) drug. In some examples, the antibody as described herein or pharmaceutical composition comprising the antibody as described herein is administered in combination with anti-inflammatory medication. In some examples, the antibody as described herein or pharmaceutical composition comprising the antibody as described herein is administered in combination with a Type I interferon. In some examples, the antibody as described herein or pharmaceutical composition comprising the antibody as described herein is administered in combination with a Type II interferon. In some examples, the antibody as described herein or pharmaceutical composition comprising the antibody as described herein is administered in combination with a Type III interferon. In some examples, the antibody as described herein or pharmaceutical composition comprising the antibody as described herein is administered in combination with another drug that reduces COVID-19-related death. In some examples, the antibody as described herein or pharmaceutical composition comprising the antibody as described herein is administered in combination with another drug that reduces COVID-19-related induced inflammation. In some examples, the antibody as described herein or pharmaceutical composition comprising the antibody as described herein is administered in combination with another drug that reduces severity or disease progression from mild to severe for COVID-19.

In another example, a kit for treating SARS-CoV-2 related diseases, such as COVID-19, is provided, wherein the kit includes an antibody as described herein and instructions to administer the antibody to a subject in need of treatment. There is also provided a pharmaceutical or diagnostic pack or kit comprising one or more containers filled with one or more of the ingredients of the pharmaceutical compositions as disclosed herein, such as one or more anti-SARS-CoV-2 antibodies provided herein. Optionally associated with such container(s) can be a notice in the form prescribed by a governmental agency regulating the manufacture, use or sale of pharmaceuticals or biological products, which notice reflects approval by the agency of manufacture, use or sale for human administration, e.g., an authorisation number.

In another example, an article of manufacture that includes a container in which a composition containing an antibody as described herein and a packaging insert or label indicating that the composition can be used to treat a SARS-CoV-2 related disease, such as COVID-19, is provided. In one example, there is provided a kit for treating and/or preventing a SARS-CoV-2 related disease, such as COVID-19, the kit comprising an antibody as disclosed herein in any example or combination of examples (and optionally a further therapeutic agent as described elsewhere herein) optionally in combination with a label or instructions for use to treat and/or prevent said disease or condition in a human; optionally wherein the label or instructions comprise a marketing authorisation number (e.g., an FDA or EMA authorisation number); optionally wherein the kit comprises an IV or injection device that comprises the antibody. In another example, the kit comprises an antibody contained within a container or an IV bag. In another example, the container or IV bag is a sterile container or a sterile IV bag. In another example, the antibody is formulated into a pharmaceutical composition contained within a (sterile) container or contained within a (sterile) IV bag. In a further example, the kit further comprises instructions for use.

In another example, a kit for treating SARS-CoV-2 related diseases, such as COVID-19, is provided, wherein the kit includes an antibody as described herein and instructions to administer the antibody to a subject in need of treatment. The subject in need is specifically defined in the kit as someone of a specific higher risk group defined by epidemiological data, risk stratification data from the person's health records, risk stratification by the genotype of certain genes of the individual or the presence of certain biomarkers in the person's blood or other tissue sample. Where the risk stratification involves another pharmaceutical or diagnostic pack or kit, the combined product will act as a linked diagnostic/prognostic and treatment kit.

### Prevention of Infection—Prophylatic Use

Antibodies as described herein may be used prophylactically. Administration of antibodies may prevent infection or reduce the risk of infection by SARS-CoV-2. Antibodies may for example be used to prevent infection in those at risk in high transmission environments and to prevent infection in those unable to be vaccinated or where vaccine efficacy is low.
1. It is estimated in the UK about hundreds of thousands of people may not be able to be vaccinated due to underlying co-morbidities and immune deficiencies.
2. Vaccine efficacy is known to decrease gradually in people older the 50 years old. In the UK 18% of the population (˜12 million people) are older than 65 years old.
   Often, group 1 and group 2 overlap. These define risk groups for mAb prophylaxsis during peak SARS-CoV-2 transmission. A relevant end point is decreased rate of infection in the risk group(s).

### Diagnostics

Antibodies as described herein can be used to detect the presence, absence and/or level of SARS-CoV-2 in a biological sample from a patient. In one example, the biological sample is a tissue sample (e.g., in pathology studies or biopsy samples of tissue used for diagnostics and prognostics). In other examples, the biological sample is blood, plasma, serum, urine, faeces, cerebrospinal fluid (CFS). In other examples, the biological sample is from a nasal or throat swab. Liquid samples are convenient for use in many types of diagnostic assays.

The antibodies described herein can be used to identify the presence, absence and/or level of SARS-CoV-2 at baseline, i.e., before treatment.

The antibodies described herein can be used to guide therapy, particularly to identify the presence, absence and/or level of SARS-CoV-2 during or after treatment.

The antibodies described herein can be used for patient monitoring, to help evaluate whether a course of treatment is effective and whether or not treatment should be continued.

In one example, the antibody described herein is labelled with a detectable moiety, for example, a radiolabel, fluorescent label, enzymatic label, chemiluminescent labelled or a biotinyl group. Radioisotopes or radionuclides may include 3H, 14C, 15N, 35S, 90Y, 99Tc, 115In, 1251, 1311, fluorescent labels may include rhodamine, lanthanide phosphors or FITC and enzymatic labels may include horseradish peroxidase, β-galactosidase, luciferase, alkaline phosphatase. Additional labels include, by way of illustration and not limitation: enzymes, such as glucose-6-phosphate dehydrogenase (“G6PDH”), alpha-D-galactosidase, glucose oxydase, glucose amylase, carbonic anhydrase, acetylcholinesterase, lysozyme, malate dehydrogenase and peroxidase; dyes; additional fluorescent labels or fluorescers include, such as fluorescein and its derivatives, fluorochrome, GFP (GFP for “Green Fluorescent Protein”), dansyl, umbelliferone, phycoerythrin, phycocyanin, allophycocyanin, o-phthaldehyde, and fiuorescamine; fluorophores such as lanthanide cryptates and chelates e.g. Europium etc (Perkin Elmer and Cisbio Assays); chemoluminescent labels or chemiluminescers, such as isoluminol, luminol and the dioxetanes; sensitisers; coenzymes; enzyme substrates; particles, such as latex or carbon particles; metal sol; crystallite; liposomes; cells, etc., which may be further labelled with a dye, catalyst or other detectable group; molecules such as biotin, digoxygenin or 5-bromodeoxyuridine; toxin moieties, such as for example a toxin moiety selected from a group of Pseudomonas exotoxin (PE or a cytotoxic fragment or mutant thereof), Diptheria toxin or a cytotoxic fragment or mutant thereof, a botulinum toxin A, B, C, D, E or F, ricin or a cytotoxic fragment thereof e.g. ricin A, abrin or a cytotoxic fragment thereof, saporin or a cytotoxic fragment thereof, pokeweed antiviral toxin or a cytotoxic fragment thereof and bryodin 1 or a cytotoxic fragment thereof

In one example, the antibody can be administered to a patient, wherein the antibody is conjugated to a label. The presence of the label in the patient can be measured or observed, wherein a relatively high amount of the label may indicate a high risk of disease and a relatively low amount of the label may indicate a relatively low risk of the disease. In one example, the label is a contrast agent, isotopic tag, or fluorescent marker, such as green fluorescent protein.

For use in diagnostics, antibodies with high affinity, especially with fast on-rate and slow off-rate (e.g., as measured by SPR) are particularly valuable.

In some embodiments, it is desirable to include 2 antibodies in a diagnostic assay and these should preferably be directed to different regions of the spike protein. The diagnostic assay could be a double antigen binding assay (DABA). In a DABA, a first antibody is used as a capture antibody to bind the virus or spike protein in a sample (for this purpose, a high affinity antibody with fast on-rate and slow off-rate is desirable, as noted above), and a second antibody, specific for an epitope that is different from the capture antibody's epitope, is used for detection. The second antibody may thus be detectably labelled, by direct or indirect labelling. A DABA may comprise providing the first antibody (optionally immobilised on a surface), contacting the surface with a sample to allow capture of antigen, if present, followed by washing to remove unbound antigen and sample, and then exposing the surface to the detection antibody to allow binding to the antigen, if present, washing to remove unbound detection antibody, and detecting the presence of the detection antibody. The presence of the detection antibody indicates that the sample is positive for the spike protein. This type of assay may be used to determine whether a patient is infected with the virus.

In other embodiments, a diagnostic assay may employ neutralising antibodies (e.g., an antibody that neutralises binding of spike protein to ACE2) as competitive antibodies to determine the level of neutralising antibodies in the serum of convalescent patients, vaccinated individuals or those who were previously infected with SARS-CoV-2. The neutralising monoclonal antibody is supplied in the assay in excess, and competition with antibodies in the sample is assessed. Detection of competition is indicative of the presence of neutralising antibody in the sample.

In one example, a kit for detecting SARS-CoV-2 in a biological sample is provided. The kit can be used to screen for SARS-CoV-2 related diseases. In one example, the kit includes an antibody according to the invention as described anywhere herein and a means for determining whether the antibody is bound to SARS-CoV-2 in a sample. In one example, the antibody is specific for SARS-CoV-2. In one example, the antibody is labelled. In another example, the antibody is an unlabelled primary antibody and the kit includes means for detecting the primary antibody. In one example, the means for detecting includes a labelled secondary antibody that is an anti-immunoglobulin antibody. The antibody may be labelled with any suitable marker, including, for example, a fluorochrome, an enzyme, a radionuclide and a radiopaque material.

In one example, the primary antibody is an antibody that specifically binds to the RBD of SARS-CoV-2 spike protein and does not compete for binding with the ACE2 receptor and the secondary antibody is an antibody that specifically binds to the RBD of SARS-CoV-2 spike protein and does compete for binding with the ACE2 receptor.

In one example, a kit for detecting SARS-CoV-2 is provided, wherein the kit includes an antibody as described herein. In one example, the kit may also include instructions and one or more reagents for detecting SARS-CoV-2.

### CLAUSES

Aspects of the invention are disclosed in the following lettered and numbered clauses:

A1. An antibody that specifically binds to the receptor binding domain (RBD) of the SARS-CoV-2 spike protein, wherein the antibody competes for binding to the SARS-CoV-2 spike protein with the human ACE2 receptor.

A2. An antibody according to clause A1,
wherein the antibody binds the isolated RBD of the SARS-CoV-2 spike protein with a KD of 10-9 M or lower (e.g. as measured by surface plasmon resonance (SPR)).

A3. An antibody according to clause A1,
wherein the antibody neutralises SARS-CoV-2 with an IC50 of 50 pM or lower (e.g. as measured in a pseudovirus neutralisation assay).

A4. An antibody according to clause A2 or clause A3,
wherein the antibody binds the isolated RBD of the SARS-CoV-2 spike protein with a KD of 10-9 M or lower (e.g. as measured by surface plasmon resonance (SPR)) and wherein the antibody neutralises SARS-CoV-2 with an IC50 of 50 pM or lower (e.g. as measured in a pseudovirus neutralisation assay).

A5. An antibody according to any one of clauses A1 to A4,
wherein the antibody comprises a variable heavy (VH) domain sequence comprising complementarity determining regions (CDRs) HCDR1, HCDR2 and HCDR3, and a variable light (VL) domain sequence comprising complementarity determining regions LCDR1, LCDR2 and LCDR3,
wherein the HCDR3 is the HCDR3 of antibody IMPI-029, IMPI-056, IMPI-005, IMPI-012, IMPI-052, IMPI-002, IMPI-041, IMPI-036, IMPI-055, IMPI-054, IMPI-042, IMPI-021, IMPI-004, IMPI-047, IMPI-017, IMPI-059, or IMPI-028.

A6. An antibody according to clause A2,
wherein the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3,
wherein the HCDR3 is the HCDR3 of antibody IMPI-004, IMPI-029, IMPI-056, IMPI-055, or IMPI-059.

A7. An antibody according to clause A3,
wherein the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3,
wherein the HCDR3 is the HCDR3 of antibody IMPI-004, IMPI-029, IMPI-055, IMPI-059, or IMPI-017.

A8. An antibody according to clause A4,
wherein the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3,
wherein the HCDR3 is the HCDR3 of antibody IMPI-004, IMPI-029, IMPI-055, or IMPI-059.

A9. The antibody according to clause A5, wherein the HCDR3 is the HCDR3 of antibody IMPI-029.

A10. The antibody according to clause A5, wherein the HCDR3 is the HCDR3 of antibody IMPI-056.

A11. The antibody according to clause A5, wherein the HCDR3 is the HCDR3 of antibody IMPI-005.

A12. The antibody according to clause A5, wherein the HCDR3 is the HCDR3 of antibody IMPI-012.

A13. The antibody according to clause A5, wherein the HCDR3 is the HCDR3 of antibody IMPI-052.

A14. The antibody according to clause A5, wherein the HCDR3 is the HCDR3 of antibody IMPI-002.

A15. The antibody according to clause A5, wherein the HCDR3 is the HCDR3 of antibody IMPI-041.

A16. The antibody according to clause A5, wherein the HCDR3 is the HCDR3 of antibody IMPI-036.

A17. The antibody according to clause A5, wherein the HCDR3 is the HCDR3 of antibody IMPI-055.

A18. The antibody according to clause A5, wherein the HCDR3 is the HCDR3 of antibody IMPI-054.

A19. The antibody according to clause A5, wherein the HCDR3 is the HCDR3 of antibody IMPI-042.

A20. The antibody according to clause A5, wherein the HCDR3 is the HCDR3 of antibody IMPI-021.

A21. The antibody according to clause A5, wherein the HCDR3 is the HCDR3 of antibody IMPI-004.

A22. The antibody according to clause A5, wherein the HCDR3 is the HCDR3 of antibody IMPI-047.

A23. The antibody according to clause A5, wherein the HCDR3 is the HCDR3 of antibody IMPI-017.

A24. The antibody according to clause A5, wherein the HCDR3 is the HCDR3 of antibody IMPI-059.

A25. The antibody according to clause A5, wherein the HCDR3 is the HCDR3 of antibody IMPI-028.

A26. An anti-SARS-CoV-2 antibody,
wherein the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3,
wherein the CDRs are those of antibody IMPI-029, IMPI-056, IMPI-005, IMPI-012, IMPI-052, IMPI-002, IMPI-041, IMPI-036, IMPI-055, IMPI-054, IMPI-042, IMPI-021, IMPI-004, IMPI-047, IMPI-017, IMPI-059, or IMPI-028.

A27. An anti-SARS-CoV-2 antibody which competes for binding to the SARS-CoV-2 spike protein with the human ACE2 receptor,
wherein the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3,
wherein the CDRs are those of antibody IMPI-029, IMPI-056, IMPI-005, IMPI-012, IMPI-052, IMPI-002, IMPI-041, IMPI-036, IMPI-055, IMPI-054, IMPI-042, IMPI-021, IMPI-004, IMPI-047, IMPI-017, IMPI-059, or IMPI-028.

A28. An antibody according to any one of clauses A1 to A4,
wherein the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3,
wherein the CDRs are those of antibody IMPI-029, IMPI-056, IMPI-005, IMPI-012, IMPI-052, IMPI-002, IMPI-041, IMPI-036, IMPI-055, IMPI-054, IMPI-042, IMPI-021, IMPI-004, IMPI-047, IMPI-017, IMPI-059, or IMPI-028.

A29. An antibody according to clause A2,
wherein the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3,
wherein the CDRs are those of antibody IMPI-004, IMPI-029, IMPI-056, IMPI-055, or IMPI-059.

A30. An antibody according to clause A3,
wherein the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3,
wherein the CDRs are those of antibody IMPI-004, IMPI-029, IMPI-055, IMPI-059 or IMPI-017.

A31. An antibody according to clauseA4,
wherein the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3,
wherein the CDRs are those of antibody IMPI-004, IMPI-029, IMPI-055 or IMPI-059.

A32. The antibody according to any one of clauses A26 to A28, wherein the antibody has the CDRs of antibody IMPI-029.

A33. The antibody according to any one of clauses A26 to A28, wherein the antibody has the CDRs of antibody IMPI-056.

A34. The antibody according to any one of clauses A26 to A28, wherein the antibody has the CDRs of antibody IMPI-005.

A35. The antibody according to any one of clauses A26 to A28, wherein the antibody has the CDRs of antibody IMPI-012.

A36. The antibody according to any one of clauses A26 to A28, wherein the antibody has the CDRs of antibody IMPI-052.

A37. The antibody according to any one of clauses A26 to A28, wherein the antibody has the CDRs of antibody IMPI-002.

A38. The antibody according to any one of clauses A26 to A28, wherein the antibody has the CDRs of antibody IMPI-041.

A39. The antibody according to any one of clauses A26 to A28, wherein the antibody has the CDRs of antibody IMPI-036.

A40. The antibody according to any one of clauses A26 to A28, wherein the antibody has the CDRs of antibody IMPI-055.

A41. The antibody according to any one of clauses A26 to A28, wherein the antibody has the CDRs of antibody IMPI-054.

A42. The antibody according to any one of clauses A26 to A28, wherein the antibody has the CDRs of antibody IMPI-042.

A43. The antibody according to any one of clauses A26 to A28, wherein the antibody has the CDRs of antibody IMPI-021.

A44. The antibody according to any one of clauses A26 to A28, wherein the antibody has the CDRs of antibody IMPI-004.

A45. The antibody according to any one of clauses A26 to A28, wherein the antibody has the CDRs of antibody IMPI-047.

A46. The antibody according to any one of clauses A26 to A28, wherein the antibody has the CDRs of antibody IMPI-017.

A47. The antibody according to any one of clauses A26 to A28, wherein the antibody has the CDRs of antibody IMPI-059.

A48. The antibody according to any one of clauses A26 to A28, wherein the antibody has the CDRs of antibody IMPI-028.

A49. An antibody according to any one of clauses A1 to A4,
wherein the antibody comprises a variable heavy (VH) domain sequence and a variable light (VL) domain sequence and wherein the variable heavy (VH) domain and variable light (VL) domain sequences respectively comprise the variable heavy (VH) domain and variable light (VL) domain sequences of antibody IMPI-029, IMPI-056, IMPI-005, IMPI-012, IMPI-052, IMPI-002, IMPI-041, IMPI-036, IMPI-055, IMPI-054, IMPI-042, IMPI-021, IMPI-004, IMPI-047, IMPI-017, IMPI-059, or IMPI-028, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

A50. An antibody according to clause A2,
wherein the antibody comprises a variable heavy (VH) domain sequence and a variable light (VL) domain sequence and wherein the variable heavy (VH) domain and variable light (VL) domain sequences respectively comprise the variable heavy (VH) domain and variable light (VL) domain sequences of antibody IMPI-004, IMPI-029, IMPI-056, IMPI-055 or IMPI-059, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

A51. An antibody according to clause A3,
wherein the antibody comprises a variable heavy (VH) domain sequence and a variable light (VL) domain sequence and wherein the variable heavy (VH) domain and variable light (VL) domain sequences respectively comprise the variable heavy (VH) domain and variable light (VL) domain sequences of antibody IMPI-004, IMPI-029, IMPI-055, or IMPI-059 or IMPI-017, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

A52. An antibody according to clause A4,
wherein the antibody comprises a variable heavy (VH) domain sequence and a variable light (VL) domain sequence and wherein the variable heavy (VH) domain and variable light (VL) domain sequences respectively comprise the variable heavy (VH) domain and variable light (VL) domain sequences of antibody IMPI-004, IMPI-029, IMPI-055 or IMPI-059, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

A53. The antibody according to clause A49, wherein the antibody comprises a variable heavy (VH) domain sequence of antibody IMPI-029, optionally with 1, 2, 3, 4 or 5 amino acid alterations outside the complementarity determining regions (CDRs), and a variable light (VL) domain sequence of antibody IMPI-029, optionally with 1, 2, 3, 4 or 5 amino acid alterations outside the complementarity determining regions (CDRs).

A54. The antibody according to clause A49, wherein the antibody comprises a variable heavy (VH) domain sequence of antibody IMPI-056, optionally with 1, 2, 3, 4 or 5 amino acid alterations outside the complementarity determining regions (CDRs), and a variable light (VL) domain sequence of antibody IMPI-056, optionally with 1, 2, 3, 4 or 5 amino acid alterations outside the complementarity determining regions (CDRs).

A55. The antibody according to clause A49, wherein the antibody comprises a variable heavy (VH) domain sequence of antibody IMPI-005, optionally with 1, 2, 3, 4 or 5 amino acid alterations outside the complementarity determining regions (CDRs), and a variable light (VL) domain sequence of antibody IMPI-005, optionally with 1, 2, 3, 4 or 5 amino acid alterations outside the complementarity determining regions (CDRs).

A56. The antibody according to clause A49, wherein the antibody comprises a variable heavy (VH) domain sequence of antibody IMPI-012, optionally with 1, 2, 3, 4 or 5 amino acid alterations outside the complementarity determining regions (CDRs), and a variable light (VL) domain sequence of antibody IMPI-012, optionally with 1, 2, 3, 4 or 5 amino acid alterations outside the complementarity determining regions (CDRs).

A57. The antibody according to clause A49, wherein the antibody comprises a variable heavy (VH) domain sequence of antibody IMPI-052, optionally with 1, 2, 3, 4 or 5 amino acid alterations outside the complementarity determining regions (CDRs), and a variable light (VL) domain sequence of antibody IMPI-052, optionally with 1, 2, 3, 4 or 5 amino acid alterations outside the complementarity determining regions (CDRs).

A58. The antibody according to clause A49, wherein the antibody comprises a variable heavy (VH) domain sequence of antibody IMPI-002, optionally with 1, 2, 3, 4 or 5 amino acid alterations outside the complementarity determining regions (CDRs), and a variable light (VL) domain sequence of antibody IMPI-002, optionally with 1, 2, 3, 4 or 5 amino acid alterations outside the complementarity determining regions (CDRs).

A59. The antibody according to clause A49, wherein the antibody comprises a variable heavy (VH) domain sequence of antibody IMPI-041, optionally with 1, 2, 3, 4 or 5 amino acid alterations outside the complementarity determining regions (CDRs), and a variable light (VL) domain sequence of antibody IMPI-041, optionally with 1, 2, 3, 4 or 5 amino acid alterations outside the complementarity determining regions (CDRs).

A60. The antibody according to clause A49, wherein the antibody comprises a variable heavy (VH) domain sequence of antibody IMPI-036, optionally with 1, 2, 3, 4 or 5 amino acid alterations outside the complementarity determining regions (CDRs), and a variable light (VL) domain sequence of antibody IMPI-036, optionally with 1, 2, 3, 4 or 5 amino acid alterations outside the complementarity determining regions (CDRs).

A61. The antibody according to clause A49, wherein the antibody comprises a variable heavy (VH) domain sequence of antibody IMPI-055, optionally with 1, 2, 3, 4 or 5 amino acid alterations outside the complementarity determining regions (CDRs), and a variable light (VL) domain sequence of antibody IMPI-055, optionally with 1, 2, 3, 4 or 5 amino acid alterations outside the complementarity determining regions (CDRs).

A62. The antibody according to clause A49, wherein the antibody comprises a variable heavy (VH) domain sequence of antibody IMPI-054, optionally with 1, 2, 3, 4 or 5 amino acid alterations outside the complementarity determining regions (CDRs), and a variable light (VL) domain sequence of antibody IMPI-054, optionally with 1, 2, 3, 4 or 5 amino acid alterations outside the complementarity determining regions (CDRs).

A63. The antibody according to clause A49, wherein the antibody comprises a variable heavy (VH) domain sequence of antibody IMPI-042, optionally with 1, 2, 3, 4 or 5 amino acid alterations outside the complementarity determining regions (CDRs), and a variable light (VL) domain sequence of antibody IMPI-042, optionally with 1, 2, 3, 4 or 5 amino acid alterations outside the complementarity determining regions (CDRs).

A64. The antibody according to clause A49, wherein the antibody comprises a variable heavy (VH) domain sequence of antibody IMPI-021, optionally with 1, 2, 3, 4 or 5 amino acid alterations outside the complementarity determining regions (CDRs), and a variable light (VL) domain sequence of antibody IMPI-021, optionally with 1, 2, 3, 4 or 5 amino acid alterations outside the complementarity determining regions (CDRs).

A65. The antibody according to clause A49, wherein the antibody comprises a variable heavy (VH) domain sequence of antibody IMPI-004, optionally with 1, 2, 3, 4 or 5 amino acid alterations outside the complementarity determining regions (CDRs), and a variable light (VL) domain sequence of antibody IMPI-004, optionally with 1, 2, 3, 4 or 5 amino acid alterations outside the complementarity determining regions (CDRs).

A66. The antibody according to clause A49, wherein the antibody comprises a variable heavy (VH) domain sequence of antibody IMPI-047, optionally with 1, 2, 3, 4 or 5 amino acid alterations outside the complementarity determining regions (CDRs), and a variable light (VL) domain sequence of antibody IMPI-047, optionally with 1, 2, 3, 4 or 5 amino acid alterations outside the complementarity determining regions (CDRs).

A67. The antibody according to clause A49, wherein the antibody comprises a variable heavy (VH) domain sequence of antibody IMPI-017, optionally with 1, 2, 3, 4 or 5 amino acid alterations outside the complementarity determining regions (CDRs), and a variable light (VL) domain sequence of antibody IMPI-017, optionally with 1, 2, 3, 4 or 5 amino acid alterations outside the complementarity determining regions (CDRs).

A68. The antibody according to clause A49, wherein the antibody comprises a variable heavy (VH) domain sequence of antibody IMPI-059, optionally with 1, 2, 3, 4 or 5 amino acid alterations outside the complementarity determining regions (CDRs), and a variable light (VL) domain sequence of antibody IMPI-059, optionally with 1, 2, 3, 4 or 5 amino acid alterations outside the complementarity determining regions (CDRs).

A69. The antibody according to clause A49, wherein the antibody comprises a variable heavy (VH) domain sequence of antibody IMPI-028, optionally with 1, 2, 3, 4 or 5 amino acid alterations outside the complementarity determining regions (CDRs), and a variable light (VL) domain sequence of antibody IMPI-028, optionally with 1, 2, 3, 4 or 5 amino acid alterations outside the complementarity determining regions (CDRs).

A70. An antibody according to any one of clauses A1 to A4,
wherein the antibody comprises a variable heavy (VH) domain sequence and a variable light (VL) domain sequence and wherein the variable heavy (VH) domain and variable light (VL) domain sequences respectively comprise a sequence having at least 90% identity to the variable heavy (VH) and variable light (VL) domain sequences of antibody IMPI-029, IMPI-056, IMPI-005, IMPI-012, IMPI-052, IMPI-002, IMPI-041, IMPI-036, IMPI-055, IMPI-054, IMPI-042, IMPI-021, IMPI-004, IMPI-047, IMPI-017, IMPI-059, or IMPI-028, provided that the antibody has the CDRs of antibody IMPI-029, IMPI-056, IMPI-005, IMPI-012, IMPI-052, IMPI-002, IMPI-041, IMPI-036, IMPI-055, IMPI-054, IMPI-042, IMPI-021, IMPI-004, IMPI-047, IMPI-017, IMPI-059, or IMPI-028.

A71. An antibody according to clause A2,
wherein the antibody comprises a variable heavy (VH) domain sequence and a variable light (VL) domain sequence and wherein the variable heavy (VH) domain and variable light (VL) domain sequences respectively comprise a sequence having at least 90% identity to the variable heavy (VH) and variable light (VL) domain sequences of antibody IMPI-004, IMPI-029, IMPI-056, IMPI-055 or IMPI-059, provided that the antibody has the CDRs of antibody IMPI-004, IMPI-029, IMPI-056, IMPI-055 or IMPI-059.

A72. An antibody according to clause A3,
wherein the antibody comprises a variable heavy (VH) domain sequence and a variable light (VL) domain sequence and wherein the variable heavy (VH) domain and variable light (VL) domain sequences respectively comprise a sequence having at least 90% identity to the variable heavy (VH) and variable light (VL) domain sequences of antibody IMPI-004, IMPI-029, IMPI-055, or IMPI-059 or IMPI-017, provided that the antibody has the CDRs of antibody IMPI-004, IMPI-029, IMPI-055, or IMPI-059 or IMPI-017.

A73. An antibody according to clause A4,
wherein the antibody comprises a variable heavy (VH) domain sequence and a variable light (VL) domain sequence and wherein the variable heavy (VH) domain and variable light (VL) domain sequences respectively comprise a sequence having at least 90% identity to the variable heavy (VH) and variable light (VL) domain sequences of antibody IMPI-004, IMPI-029, IMPI-055 or IMPI-059, provided that the antibody has the CDRs of antibody IMPI-004, IMPI-029, IMPI-055 or IMPI-059.

A74. The antibody according to clause A70, wherein the variable heavy (VH) domain sequence comprises a sequence having at least 90% identity to the VH domain sequence of antibody IMPI-029 and the variable light (VL) domain sequence comprises a sequence having at least 90% identity to the VL domain sequence of antibody IMPI-029, provided that the antibody has the CDRs of antibody IMPI-029.

A75. The antibody according to clause A70, wherein the variable heavy (VH) domain sequence comprises a sequence having at least 90% identity to the VH domain sequence of antibody IMPI-056 and the variable light (VL) domain sequence comprises a sequence having at least 90% identity to the VL domain sequence of antibody IMPI-056, provided that the antibody has the CDRs of antibody IMPI-056.

A76. The antibody according to clause A70, wherein the variable heavy (VH) domain sequence comprises a sequence having at least 90% identity to the VH domain sequence of antibody IMPI-005 and the variable light (VL) domain sequence comprises a sequence having at least 90% identity to the VL domain sequence of antibody IMPI-005, provided that the antibody has the CDRs of antibody IMPI-005.

A77. The antibody according to clause A70, wherein the variable heavy (VH) domain sequence comprises a sequence having at least 90% identity to the VH domain sequence of antibody IMPI-012 and the variable light (VL) domain sequence comprises a sequence having at least 90% identity to the VL domain sequence of antibody IMPI-012, provided that the antibody has the CDRs of antibody IMPI-012.

A78. The antibody according to clause A70, wherein the variable heavy (VH) domain sequence comprises a sequence having at least 90% identity to the VH domain sequence of antibody IMPI-052 and the variable light (VL) domain sequence comprises a sequence having at least 90% identity to the VL domain sequence of antibody IMPI-052, provided that the antibody has the CDRs of antibody IMPI-052.

A79. The antibody according to clause A70, wherein the variable heavy (VH) domain sequence comprises a sequence having at least 90% identity to the VH domain sequence of antibody IMPI-002 and the variable light (VL) domain sequence comprises a sequence having at least 90% identity to the VL domain sequence of antibody IMPI-002, provided that the antibody has the CDRs of antibody IMPI-002.

A80. The antibody according to clause A70, wherein the variable heavy (VH) domain sequence comprises a sequence having at least 90% identity to the VH domain sequence of antibody IMPI-041 and the variable light (VL) domain sequence comprises a sequence having at least 90% identity to the VL domain sequence of antibody IMPI-041, provided that the antibody has the CDRs of antibody IMPI-041.

A81. The antibody according to clause A70, wherein the variable heavy (VH) domain sequence comprises a sequence having at least 90% identity to the VH domain sequence of antibody IMPI-036 and the variable light (VL) domain sequence comprises a sequence having at least 90% identity to the VL domain sequence of antibody IMPI-036, provided that the antibody has the CDRs of antibody IMPI-036.

A82. The antibody according to clause A70, wherein the variable heavy (VH) domain sequence comprises a sequence having at least 90% identity to the VH domain sequence of antibody IMPI-055 and the variable light (VL) domain sequence comprises a sequence having at least 90% identity to the VL domain sequence of antibody IMPI-055, provided that the antibody has the CDRs of antibody IMPI-055.

A83. The antibody according to clause A70, wherein the variable heavy (VH) domain sequence comprises a sequence having at least 90% identity to the VH domain sequence of antibody IMPI-054 and the variable light (VL) domain sequence comprises a sequence having at least 90% identity to the VL domain sequence of antibody IMPI-054, provided that the antibody has the CDRs of antibody IMPI-054.

A84. The antibody according to clause A70, wherein the variable heavy (VH) domain sequence comprises a sequence having at least 90% identity to the VH domain sequence of antibody IMPI-042 and the variable light (VL) domain sequence comprises a sequence having at least 90% identity to the VL domain sequence of antibody IMPI-042, provided that the antibody has the CDRs of antibody IMPI-042.

A85. The antibody according to clause A70, wherein the variable heavy (VH) domain sequence comprises a sequence having at least 90% identity to the VH domain sequence of antibody IMPI-021 and the variable light (VL) domain sequence comprises a sequence having at least 90% identity to the VL domain sequence of antibody IMPI-021, provided that the antibody has the CDRs of antibody IMPI-021.

A86. The antibody according to clause A70, wherein the variable heavy (VH) domain sequence comprises a sequence having at least 90% identity to the VH domain sequence of antibody IMPI-004 and the variable light (VL) domain sequence comprises a sequence having at least 90% identity to the VL domain sequence of antibody IMPI-004, provided that the antibody has the CDRs of antibody IMPI-004.

A87. The antibody according to clause A70, wherein the variable heavy (VH) domain sequence comprises a sequence having at least 90% identity to the VH domain sequence of antibody IMPI-047 and the variable light (VL) domain sequence comprises a sequence having at least 90% identity to the VL domain sequence of antibody IMPI-047, provided that the antibody has the CDRs of antibody IMPI-047.

A88. The antibody according to clause A70, wherein the variable heavy (VH) domain sequence comprises a sequence having at least 90% identity to the VH domain sequence of antibody IMPI-017 and the variable light (VL) domain sequence comprises a sequence having at least 90% identity to the VL domain sequence of antibody IMPI-017, provided that the antibody has the CDRs of antibody IMPI-017.

A89. The antibody according to clause A70, wherein the variable heavy (VH) domain sequence comprises a sequence having at least 90% identity to the VH domain sequence of antibody IMPI-059 and the variable light (VL) domain sequence comprises a sequence having at least 90% identity to the VL domain sequence of antibody IMPI-059, provided that the antibody has the CDRs of antibody IMPI-059.

A90. The antibody according to clause A70, wherein the variable heavy (VH) domain sequence comprises a sequence having at least 90% identity to the VH domain sequence of antibody IMPI-028 and the variable light (VL) domain sequence comprises a sequence having at least 90% identity to the VL domain sequence of antibody IMPI-028, provided that the antibody has the CDRs of antibody IMPI-028.

A91. An antibody to any one of clauses A1 to A4, wherein the antibody comprises a variable heavy (VH) domain sequence and a variable light (VL) domain sequence and wherein the variable heavy (VH) domain and variable light (VL) domain sequences respectively comprise the variable heavy (VH) and variable light (VL) domain sequences of antibody IMPI-029, IMPI-056, IMPI-005, IMPI-012, IMPI-052, IMPI-002, IMPI-041, IMPI-036, IMPI-055, IMPI-054, IMPI-042, IMPI-021, IMPI-004, IMPI-047, IMPI-017, IMPI-059, or IMPI-028.

A92. An antibody that specifically binds to the receptor binding domain (RBD) of the SARS-CoV-2 spike protein, wherein the antibody comprises a variable heavy (VH) domain sequence and a variable light (VL) domain sequence and wherein the variable heavy (VH) domain and variable light (VL) domain sequences respectively comprise the variable heavy (VH) and variable light (VL) domain sequences of antibody IMPI-029, IMPI-056, IMPI-005, IMPI-012, IMPI-052, IMPI-002, IMPI-041, IMPI-036, IMPI-055, IMPI-054, IMPI-042, IMPI-021, IMPI-004, IMPI-047, IMPI-017, IMPI-059, or IMPI-028.

A93. The antibody according to clause A91 or clause A92, wherein the antibody comprises the VH domain and VL domain sequences of antibody IMPI-029.

A94. The antibody according to clause A91 or clause A92, wherein the antibody comprises the VH domain and VL domain sequences of antibody IMPI-056.

A95. The antibody according to clause A91 or clause A92, wherein the antibody comprises the VH domain and VL domain sequences of antibody IMPI-005.

A96. The antibody according to clause A91 or clause A92, wherein the antibody comprises the VH domain and VL domain sequences of antibody IMPI-012.

A97. The antibody according to clause A91 or clause A92, wherein the antibody comprises the VH domain and VL domain sequences of antibody IMPI-052.

A98. The antibody according to clause A91 or clause A92, wherein the antibody comprises the VH domain and VL domain sequences of antibody IMPI-002.

A99. The antibody according to clause A91 or clause A92, wherein the antibody comprises the VH domain and VL domain sequences of antibody IMPI-041.

A100. The antibody according to clause A91 or clause A92, wherein the antibody comprises the VH domain and VL domain sequences of antibody IMPI-036.

A101. The antibody according to clause A91 or clause A92, wherein the antibody comprises the VH domain and VL domain sequences of antibody IMPI-055.

A102. The antibody according to clause A91 or clause A92, wherein the antibody comprises the VH domain and VL domain sequences of antibody IMPI-054.

A103. The antibody according to clause A91 or clause A92, wherein the antibody comprises the VH domain and VL domain sequences of antibody IMPI-042.

A104. The antibody according to clause A91 or clause A92, wherein the antibody comprises the VH domain and VL domain sequences of antibody IMPI-021.

A105. The antibody according to clause A91 or clause A92, wherein the antibody comprises the VH domain and VL domain sequences of antibody IMPI-004.

A106. The antibody according to clause A91 or clause A92, wherein the antibody comprises the VH domain and VL domain sequences of antibody IMPI-047.

A107. The antibody according to clause A91 or clause A92, wherein the antibody comprises the VH domain and VL domain sequences of antibody IMPI-017.

A108. The antibody according to clause A91 or clause A92, wherein the antibody comprises the VH domain and VL domain sequences of antibody IMPI-059.

A109. The antibody according to clause A91 or clause A92, wherein the antibody comprises the VH domain and VL domain sequences of antibody IMPI-028.

A110. The antibody according to any one of clauses A1 to A4, comprising VH and/or VL domain framework regions of human germline gene segment sequences.

A111. The antibody according to any one of clauses A1 to A4 or A110, comprising an antibody VH domain which
i) is derived from recombination of a human heavy chain V gene segment, a human heavy chain D gene segment and a human heavy chain J gene segment, wherein
   the V gene segment is IGHV3-53*01, IGHV1-8*01 or IGHV3-33*01;
   and/or
   the J gene segment is IGHJ6*02, IGHJ4*02 or IGHJ3*02, or
ii) comprises framework regions FR1, FR2, FR3 and FR4, wherein
   FR1 aligns with human germline V gene segment IGHV3-53*01, IGHV1-8*01 or IGHV3-33*01 with up to 1, 2, 3, 4, or 5 amino acid alterations,
   FR2 aligns with human germline V gene segment IGHV3-53*01, IGHV1-8*01 or IGHV3-33*01 with up to 1, 2, 3, 4, or 5 amino acid alterations,
   FR3 aligns with human germline V gene segment IGHV3-53*01, IGHV1-8*01 or IGHV3-33*01 with up to 1, 2, 3, 4 or 5 amino acid alterations, and/or
   FR4 aligns with human germline J gene segment IGHJ6*02, IGHJ4*02 or IGHJ3*02 with up to 1, 2, 3, 4 or 5 amino acid alterations.

A112. The antibody according to any one of clauses A1 to A4, A 110 or A 111, comprising an antibody VH domain which
i) is derived from recombination of a human heavy chain V gene segment IGHV3-53*01, IGHV1-8*01 or IGHV3-33*01, a human heavy chain D gene segment and a human heavy chain J gene segment, or
ii) comprises framework regions FR1, FR2, FR3 and FR4, wherein FR1, FR2 and FR3 each align with human germline V segment IGHV3-53*01, IGHV1-8*01 or IGHV3-33*01 with up to 1, 2, 3, 4 or 5 amino acid alterations.

A113. The antibody according to any one of clauses A1 to A4 or A110 to A112, wherein the J gene segment is IGHJ6*02, IGHJ4*02 or IGHJ3*02, or wherein the VH domain framework region FR4 aligns with human germline J gene segment IGHJ6*02, IGHJ4*02 or IGHJ3*02 with 1, 2, 3, 4 or 5 amino acid alterations.

A114. The antibody according to any one of clauses A1 to A4 or A110, comprising an antibody VL domain which
i) is derived from recombination of a human light chain V gene segment and a human light chain J gene segment, wherein
   the V gene segment is IGKV1-9*d01, IGKV6-21*01, IGKV1-33*01 or IGKV3-20*01, and/or
   the J gene segment is IGKJ5*01, IGKJ4*01, IGKJ3*01, IGKJ2*04 or IGKJ1*01; or
ii) comprises framework regions FR1, FR2, FR3 and FR4, wherein
   FR1 aligns with human germline V gene segment IGKV1-9*d01, IGKV6-21*01, IGKV1-33*01 or IGKV3-20*01 with up to 1, 2, 3, 4, or 5 amino acid alterations,
   FR2 aligns with human germline V gene segment IGKV1-9*d01, IGKV6-21*01, IGKV1-33*01 or IGKV3-20*01 with up to 1, 2, 3, 4, or 5 amino acid alterations
   FR3 aligns with human germline V gene segment IGKV1-9*d01, IGKV6-21*01, IGKV1-33*01 or IGKV3-20*01 with up to 1, 2, 3, 4 or 5 amino acid alterations, and/or
   FR4 aligns with human germline J gene segment IGKJ5*01, IGKJ4*01, IGKJ3*01, IGKJ2*04 or IGKJ1*01 with up to 1, 2, 3, 4 or 5 amino acid alterations.

A115. The antibody according to any one of clauses A1 to A4 or A110, comprising an antibody VL domain derived from recombination of a human light chain V gene segment and a human light chain J gene segment, wherein:
the V gene segment is IGKV1-9*d01, IGKV6-21*01, IGKV1-33*01 or IGKV3-20*01, and optionally
the J gene segment is IGKJ5*01, IGKJ4*01, IGKJ3*01, IGKJ2*04 or IGKJ1*01.

B1. An antibody that preferentially binds to the trimer form of the SARS-CoV-2 spike protein over the isolated RBD domain, isolated S1 subunit or isolated S2 subunit of the SARS-CoV-2 spike protein.

B2. An antibody according to clause B1, wherein the antibody specifically binds to the trimer form of the SARS-CoV-2 spike protein and does not bind to the isolated RBD domain.

B3. An antibody according to clause B1 or clause B2, wherein the antibody specifically binds to the trimer form of the SARS-CoV-2 spike protein and does not bind to the isolated RBD domain, isolated S1 subunit or isolated S2 subunit of the SARS-CoV-2 spike protein.

B4. An antibody according to any one of clauses B1 to B3, wherein the antibody neutralises SARS-CoV-2 with an IC50 of 75 nM or lower, preferably 15 nM or lower (e.g. as measured in a pseudovirus neutralisation assay).

B5. An antibody according to any one of clauses B1 to B4,
wherein the antibody comprises a variable heavy (VH) domain sequence comprising complementarity determining regions (CDRs) HCDR1, HCDR2 and HCDR3, and a variable light (VL) domain sequence comprising complementarity determining regions LCDR1, LCDR2 and LCDR3,
wherein the HCDR3 is the HCDR3 of antibody IMPI-030, IMPI-053, IMPI-025, IMPI-040, IMPI-007, IMPI-020, IMPI-032, IMPI-023, IMPI-039, IMPI-001, IMPI-019, IMPI-010, IMPI-008, IMPI-031, IMPI-057, IMPI-022, IMPI-035, IMPI-067 or IMPI-072.

B6. The antibody according to clause B5, wherein the HCDR3 is the HCDR3 of antibody IMPI-030.

B7. The antibody according to clause B5, wherein the HCDR3 is the HCDR3 of antibody IMPI-053.

B8. The antibody according to clause B5, wherein the HCDR3 is the HCDR3 of antibody IMPI-025.

B9. The antibody according to clause B5, wherein the HCDR3 is the HCDR3 of antibody IMPI-040.

B10. The antibody according to clause B5, wherein the HCDR3 is the HCDR3 of antibody IMPI-007.

B 11. The antibody according to clause B5, wherein the HCDR3 is the HCDR3 of antibody IMPI-020.

B12. The antibody according to clause B5, wherein the HCDR3 is the HCDR3 of antibody IMPI-032.

B13. The antibody according to clause B5, wherein the HCDR3 is the HCDR3 of antibody IMPI-023.

B14. The antibody according to clause B5, wherein the HCDR3 is the HCDR3 of antibody IMPI-039.

B15. The antibody according to clause B5, wherein the HCDR3 is the HCDR3 of antibody IMPI-001.

B16. The antibody according to clause B5, wherein the HCDR3 is the HCDR3 of antibody IMPI-019.

B17. The antibody according to clause B5, wherein the HCDR3 is the HCDR3 of antibody IMPI-010.

B18. The antibody according to clause B5, wherein the HCDR3 is the HCDR3 of antibody IMPI-008.

B19. The antibody according to clause B5, wherein the HCDR3 is the HCDR3 of antibody IMPI-031.

B20. The antibody according to clause B5, wherein the HCDR3 is the HCDR3 of antibody IMPI-057.

B21. The antibody according to clause B5, wherein the HCDR3 is the HCDR3 of antibody IMPI-022.

B22. The antibody according to clause B5, wherein the HCDR3 is the HCDR3 of antibody IMPI-035.

B23. The antibody according to clause B5, wherein the HCDR3 is the HCDR3 of antibody IMPI-067.

B24. The antibody according to clause B5, wherein the HCDR3 is the HCDR3 of antibody IMPI-072.

B25. An anti-SARS-CoV-2 antibody,
wherein the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3,
wherein the CDRs are those of antibody IMPI-030, IMPI-053, IMPI-025, IMPI-040, IMPI-007, IMPI-020, IMPI-032, IMPI-023, IMPI-039, IMPI-001, IMPI-019, IMPI-010, IMPI-008, IMPI-031, IMPI-057, IMPI-022, IMPI-035, IMPI-067 or IMPI-072.

B26. An antibody according to any one of clauses B1 to B4,
wherein the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3,
wherein the CDRs are those of antibody IMPI-030, IMPI-053, IMPI-025, IMPI-040, IMPI-007, IMPI-020, IMPI-032, IMPI-023, IMPI-039, IMPI-001, IMPI-019, IMPI-010, IMPI-008, IMPI-031, IMPI-057, IMPI-022, IMPI-035, IMPI-067 or IMPI-072.

B27. The antibody according to clause B25 or clause B26, wherein the antibody has the CDRs of antibody IMPI-030.

B28. The antibody according to clause B25 or clause B26, wherein the antibody has the CDRs of antibody IMPI-053.

B29. The antibody according to clause B25 or clause B26, wherein the antibody has the CDRs of antibody IMPI-025.

B30. The antibody according to clause B25 or clause B26, wherein the antibody has the CDRs of antibody IMPI-040.

B31. The antibody according to clause B25 or clause B26, wherein the antibody has the CDRs of antibody IMPI-007.

B32. The antibody according to clause B25 or clause B26, wherein the antibody has the CDRs of antibody IMPI-020.

B33. The antibody according to clause B25 or clause B26, wherein the antibody has the CDRs of antibody IMPI-032.

B34. The antibody according to clause B25 or clause B26, wherein the antibody has the CDRs of antibody IMPI-023.

B35. The antibody according to clause B25 or clause B26, wherein the antibody has the CDRs of antibody IMPI-039.

B36. The antibody according to clause B25 or clause B26, wherein the antibody has the CDRs of antibody IMPI-001.

B37. The antibody according to clause B25 or clause B26, wherein the antibody has the CDRs of antibody IMPI-019.

B38. The antibody according to clause B25 or clause B26, wherein the antibody has the CDRs of antibody IMPI-010.

B39. The antibody according to clause B25 or clause B26, wherein the antibody has the CDRs of antibody IMPI-008.

B40. The antibody according to clause B25 or clause B26, wherein the antibody has the CDRs of antibody IMPI-031.

B41. The antibody according to clause B25 or clause B26, wherein the antibody has the CDRs of antibody IMPI-057.

B42. The antibody according to clause B25 or clause B26, wherein the antibody has the CDRs of antibody IMPI-022.

B43. The antibody according to clause B25 or clause B26, wherein the antibody has the CDRs of antibody IMPI-035.

B44. The antibody according to clause B25 or clause B26, wherein the antibody has the CDRs of antibody IMPI-067.

B45. The antibody according to clause B25 or clause B26, wherein the antibody has the CDRs of antibody IMPI-072.

B46. An antibody according to any one of clauses B1 to B4,
wherein the antibody comprises a variable heavy (VH) domain sequence and a variable light (VL) domain sequence and wherein the variable heavy (VH) domain and variable light (VL) domain sequences respectively comprise the variable heavy (VH) domain and variable light (VL) domain sequences of antibody IMPI-030, IMPI-053, IMPI-025, IMPI-040, IMPI-007, IMPI-020, IMPI-032, IMPI-023, IMPI-039, IMPI-001, IMPI-019, IMPI-010, IMPI-008, IMPI-031, IMPI-057, IMPI-022, IMPI-035, IMPI-067 or IMPI-072,
optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

B47. The antibody according to clause B46, wherein the antibody comprises a variable heavy (VH) domain sequence of antibody IMPI-030, optionally with 1, 2, 3, 4 or 5 amino acid alterations outside the complementarity determining regions (CDRs), and a variable light (VL) domain sequence of antibody IMPI-030, optionally with 1, 2, 3, 4 or 5 amino acid alterations outside the complementarity determining regions (CDRs).

B48. The antibody according to clause B46, wherein the antibody comprises a variable heavy (VH) domain sequence of antibody IMPI-053, optionally with 1, 2, 3, 4 or 5 amino acid alterations outside the complementarity determining regions (CDRs), and a variable light (VL) domain sequence of antibody IMPI-053, optionally with 1, 2, 3, 4 or 5 amino acid alterations outside the complementarity determining regions (CDRs).

B49. The antibody according to clause B46, wherein the antibody comprises a variable heavy (VH) domain sequence of antibody IMPI-025, optionally with 1, 2, 3, 4 or 5 amino acid alterations outside the complementarity determining regions (CDRs), and a variable light (VL) domain sequence of antibody IMPI-025, optionally with 1, 2, 3, 4 or 5 amino acid alterations outside the complementarity determining regions (CDRs).

B50. The antibody according to clause B46, wherein the antibody comprises a variable heavy (VH) domain sequence of antibody IMPI-040, optionally with 1, 2, 3, 4 or 5 amino acid alterations outside the complementarity determining regions (CDRs), and a variable light (VL) domain sequence of antibody IMPI-040, optionally with 1, 2, 3, 4 or 5 amino acid alterations outside the complementarity determining regions (CDRs).

B51. The antibody according to clause B46, wherein the antibody comprises a variable heavy (VH) domain sequence of antibody IMPI-007, optionally with 1, 2, 3, 4 or 5 amino acid alterations outside the complementarity determining regions (CDRs), and a variable light (VL) domain sequence of antibody IMPI-007, optionally with 1, 2, 3, 4 or 5 amino acid alterations outside the complementarity determining regions (CDRs).

B52. The antibody according to clause B46, wherein the antibody comprises a variable heavy (VH) domain sequence of antibody IMPI-020, optionally with 1, 2, 3, 4 or 5 amino acid alterations outside the complementarity determining regions (CDRs), and a variable light (VL) domain sequence of antibody IMPI-020, optionally with 1, 2, 3, 4 or 5 amino acid alterations outside the complementarity determining regions (CDRs).

B53. The antibody according to clause B46, wherein the antibody comprises a variable heavy (VH) domain sequence of antibody IMPI-032, optionally with 1, 2, 3, 4 or 5 amino acid alterations outside the complementarity determining regions (CDRs), and a variable light (VL) domain sequence of antibody IMPI-032, optionally with 1, 2, 3, 4 or 5 amino acid alterations outside the complementarity determining regions (CDRs).

B54. The antibody according to clause B46, wherein the antibody comprises a variable heavy (VH) domain sequence of antibody IMPI-023, optionally with 1, 2, 3, 4 or 5 amino acid alterations outside the complementarity determining regions (CDRs), and a variable light (VL) domain sequence of antibody IMPI-023, optionally with 1, 2, 3, 4 or 5 amino acid alterations outside the complementarity determining regions (CDRs).

B55. The antibody according to clause B46, wherein the antibody comprises a variable heavy (VH) domain sequence of antibody IMPI-039, optionally with 1, 2, 3, 4 or 5 amino acid alterations outside the complementarity determining regions (CDRs), and a variable light (VL) domain sequence of antibody IMPI-039, optionally with 1, 2, 3, 4 or 5 amino acid alterations outside the complementarity determining regions (CDRs).

B56. The antibody according to clause B46, wherein the antibody comprises a variable heavy (VH) domain sequence of antibody IMPI-001, optionally with 1, 2, 3, 4 or 5 amino acid alterations outside the complementarity determining regions (CDRs), and a variable light (VL) domain sequence of antibody IMPI-001, optionally with 1, 2, 3, 4 or 5 amino acid alterations outside the complementarity determining regions (CDRs).

B57. The antibody according to clause B46, wherein the antibody comprises a variable heavy (VH) domain sequence of antibody IMPI-019, optionally with 1, 2, 3, 4 or 5 amino acid alterations outside the complementarity determining regions (CDRs), and a variable light (VL) domain sequence of antibody IMPI-019, optionally with 1, 2, 3, 4 or 5 amino acid alterations outside the complementarity determining regions (CDRs).

B58. The antibody according to clause B46, wherein the antibody comprises a variable heavy (VH) domain sequence of antibody IMPI-010, optionally with 1, 2, 3, 4 or 5 amino acid alterations outside the complementarity determining regions (CDRs), and a variable light (VL) domain sequence of antibody IMPI-010, optionally with 1, 2, 3, 4 or 5 amino acid alterations outside the complementarity determining regions (CDRs).

B59. The antibody according to clause B46, wherein the antibody comprises a variable heavy (VH) domain sequence of antibody IMPI-008, optionally with 1, 2, 3, 4 or 5 amino acid alterations outside the complementarity determining regions (CDRs), and a variable light (VL) domain sequence of antibody IMPI-008, optionally with 1, 2, 3, 4 or 5 amino acid alterations outside the complementarity determining regions (CDRs).

B60. The antibody according to clause B46, wherein the antibody comprises a variable heavy (VH) domain sequence of antibody IMPI-031, optionally with 1, 2, 3, 4 or 5 amino acid alterations outside the complementarity determining regions (CDRs), and a variable light (VL) domain sequence of antibody IMPI-031, optionally with 1, 2, 3, 4 or 5 amino acid alterations outside the complementarity determining regions (CDRs).

B61. The antibody according to clause B46, wherein the antibody comprises a variable heavy (VH) domain sequence of antibody IMPI-057, optionally with 1, 2, 3, 4 or 5 amino acid alterations outside the complementarity determining regions (CDRs), and a variable light (VL) domain sequence of antibody IMPI-057, optionally with 1, 2, 3, 4 or 5 amino acid alterations outside the complementarity determining regions (CDRs).

B62. The antibody according to clause B46, wherein the antibody comprises a variable heavy (VH) domain sequence of antibody IMPI-022, optionally with 1, 2, 3, 4 or 5 amino acid alterations outside the complementarity determining regions (CDRs), and a variable light (VL) domain sequence of antibody IMPI-022, optionally with 1, 2, 3, 4 or 5 amino acid alterations outside the complementarity determining regions (CDRs).

B63. The antibody according to clause B46, wherein the antibody comprises a variable heavy (VH) domain sequence of antibody IMPI-035, optionally with 1, 2, 3, 4 or 5 amino acid alterations outside the complementarity determining regions (CDRs), and a variable light (VL) domain sequence of antibody IMPI-035, optionally with 1, 2, 3, 4 or 5 amino acid alterations outside the complementarity determining regions (CDRs).

B64. The antibody according to clause B46, wherein the antibody comprises a variable heavy (VH) domain sequence of antibody IMPI-067, optionally with 1, 2, 3, 4 or 5 amino acid alterations outside the complementarity determining regions (CDRs), and a variable light (VL) domain sequence of antibody IMPI-067, optionally with 1, 2, 3, 4 or 5 amino acid alterations outside the complementarity determining regions (CDRs).

B65. The antibody according to clause B46, wherein the antibody comprises a variable heavy (VH) domain sequence of antibody IMPI-072, optionally with 1, 2, 3, 4 or 5 amino acid alterations outside the complementarity determining regions (CDRs), and a variable light (VL) domain sequence of antibody IMPI-072, optionally with 1, 2, 3, 4 or 5 amino acid alterations outside the complementarity determining regions (CDRs).

B66. An antibody according to any one of clauses B1 to B4, wherein the antibody comprises a variable heavy (VH) domain sequence and a variable light (VL) domain sequence and wherein the variable heavy (VH) domain and variable light (VL) domain sequences respectively comprise a sequence having at least 90% identity to the variable heavy (VH) and variable light (VL) domain sequences of antibody IMPI-030, IMPI-053, IMPI-025, IMPI-040, IMPI-007, IMPI-020, IMPI-032, IMPI-023, IMPI-039, IMPI-001, IMPI-019, IMPI-010, IMPI-008, IMPI-031, IMPI-057, IMPI-022, IMPI-035, IMPI-067 or IMPI-072, provided that the antibody has the CDRs of antibody IMPI-030, IMPI-053, IMPI-025, IMPI-040, IMPI-007, IMPI-020, IMPI-032, IMPI-023, IMPI-039, IMPI-001, IMPI-019, IMPI-010, IMPI-008, IMPI-031, IMPI-057, IMPI-022, IMPI-035, IMPI-067 or IMPI-072.

B67. The antibody according to clause B66, wherein the variable heavy (VH) domain sequence comprises a sequence having at least 90% identity to the VH domain sequence of antibody IMPI-030 and the variable light (VL) domain sequence comprises a sequence having at least 90% identity to the VL domain sequence of antibody IMPI-030, provided that the antibody has the CDRs of antibody IMPI-030.

B68. The antibody according to clause B66, wherein the variable heavy (VH) domain sequence comprises a sequence having at least 90% identity to the VH domain sequence of antibody IMPI-053 and the variable light (VL) domain sequence comprises a sequence having at least 90% identity to the VL domain sequence of antibody IMPI-053, provided that the antibody has the CDRs of antibody IMPI-053.

B69. The antibody according to clause B66, wherein the variable heavy (VH) domain sequence comprises a sequence having at least 90% identity to the VH domain sequence of antibody IMPI-025 and the variable light (VL) domain sequence comprises a sequence having at least 90% identity to the VL domain sequence of antibody IMPI-025, provided that the antibody has the CDRs of antibody IMPI-025.

B70. The antibody according to clause B66, wherein the variable heavy (VH) domain sequence comprises a sequence having at least 90% identity to the VH domain sequence of antibody IMPI-040 and the variable light (VL) domain sequence comprises a sequence having at least 90% identity to the VL domain sequence of antibody IMPI-040, provided that the antibody has the CDRs of antibody IMPI-040.

B71. The antibody according to clause B66, wherein the variable heavy (VH) domain sequence comprises a sequence having at least 90% identity to the VH domain sequence of antibody IMPI-007 and the variable light (VL) domain sequence comprises a sequence having at least 90% identity to the VL domain sequence of antibody IMPI-007, provided that the antibody has the CDRs of antibody IMPI-007.

B72. The antibody according to clause B66, wherein the variable heavy (VH) domain sequence comprises a sequence having at least 90% identity to the VH domain sequence of antibody IMPI-020 and the variable light (VL) domain sequence comprises a sequence having at least 90% identity to the VL domain sequence of antibody IMPI-020, provided that the antibody has the CDRs of antibody IMPI-020.

B73. The antibody according to clause B66, wherein the variable heavy (VH) domain sequence comprises a sequence having at least 90% identity to the VH domain sequence of antibody IMPI-032 and the variable light (VL) domain sequence comprises a sequence having at least 90% identity to the VL domain sequence of antibody IMPI-032, provided that the antibody has the CDRs of antibody IMPI-032.

B74. The antibody according to clause B66, wherein the variable heavy (VH) domain sequence comprises a sequence having at least 90% identity to the VH domain sequence of antibody IMPI-023 and the variable light (VL) domain sequence comprises a sequence having at least 90% identity to the VL domain sequence of antibody IMPI-023, provided that the antibody has the CDRs of antibody IMPI-023.

B75. The antibody according to clause B66, wherein the variable heavy (VH) domain sequence comprises a sequence having at least 90% identity to the VH domain sequence of antibody IMPI-039 and the variable light (VL) domain sequence comprises a sequence having at least 90% identity to the VL domain sequence of antibody IMPI-039, provided that the antibody has the CDRs of antibody IMPI-039.

B76. The antibody according to clause B66, wherein the variable heavy (VH) domain sequence comprises a sequence having at least 90% identity to the VH domain sequence of antibody IMPI-001 and the variable light (VL) domain sequence comprises a sequence having at least 90% identity to the VL domain sequence of antibody IMPI-001, provided that the antibody has the CDRs of antibody IMPI-001.

B77. The antibody according to clause B66, wherein the variable heavy (VH) domain sequence comprises a sequence having at least 90% identity to the VH domain sequence of antibody IMPI-019 and the variable light (VL) domain sequence comprises a sequence having at least 90% identity to the VL domain sequence of antibody IMPI-019, provided that the antibody has the CDRs of antibody IMPI-019.

B78. The antibody according to clause B66, wherein the variable heavy (VH) domain sequence comprises a sequence having at least 90% identity to the VH domain sequence of antibody IMPI-010 and the variable light (VL) domain sequence comprises a sequence having at least 90% identity to the VL domain sequence of antibody IMPI-010, provided that the antibody has the CDRs of antibody IMPI-010.

B79. The antibody according to clause B66, wherein the variable heavy (VH) domain sequence comprises a sequence having at least 90% identity to the VH domain sequence of antibody IMPI-008 and the variable light (VL) domain sequence comprises a sequence having at least 90% identity to the VL domain sequence of antibody IMPI-008, provided that the antibody has the CDRs of antibody IMPI-008.

B80. The antibody according to clause B66, wherein the variable heavy (VH) domain sequence comprises a sequence having at least 90% identity to the VH domain sequence of antibody IMPI-031 and the variable light (VL) domain sequence comprises a sequence having at least 90% identity to the VL domain sequence of antibody IMPI-031, provided that the antibody has the CDRs of antibody IMPI-031.

B81. The antibody according to clause B66, wherein the variable heavy (VH) domain sequence comprises a sequence having at least 90% identity to the VH domain sequence of antibody IMPI-057 and the variable light (VL) domain sequence comprises a sequence having at least 90% identity to the VL domain sequence of antibody IMPI-057, provided that the antibody has the CDRs of antibody IMPI-057.

B82. The antibody according to clause B66, wherein the variable heavy (VH) domain sequence comprises a sequence having at least 90% identity to the VH domain sequence of antibody IMPI-022 and the variable light (VL) domain sequence comprises a sequence having at least 90% identity to the VL domain sequence of antibody IMPI-022, provided that the antibody has the CDRs of antibody IMPI-022.

B83. The antibody according to clause B66, wherein the variable heavy (VH) domain sequence comprises a sequence having at least 90% identity to the VH domain sequence of antibody IMPI-035 and the variable light (VL) domain sequence comprises a sequence having at least 90% identity to the VL domain sequence of antibody IMPI-035, provided that the antibody has the CDRs of antibody IMPI-035.

B84. The antibody according to clause B66, wherein the variable heavy (VH) domain sequence comprises a sequence having at least 90% identity to the VH domain sequence of antibody IMPI-067 and the variable light (VL) domain sequence comprises a sequence having at least 90% identity to the VL domain sequence of antibody IMPI-067, provided that the antibody has the CDRs of antibody IMPI-067.

B85. The antibody according to clause B66, wherein the variable heavy (VH) domain sequence comprises a sequence having at least 90% identity to the VH domain sequence of antibody IMPI-072 and the variable light (VL) domain sequence comprises a sequence having at least 90% identity to the VL domain sequence of antibody IMPI-072, provided that the antibody has the CDRs of antibody IMPI-072.

B86. An antibody according to any one of clauses B1 to B4, wherein the antibody comprises a variable heavy (VH) domain sequence and a variable light (VL) domain sequence and wherein the variable heavy (VH) domain and variable light (VL) domain sequences respectively comprise the variable heavy (VH) and variable light (VL) domain sequences of antibody IMPI-030, IMPI-053, IMPI-025, IMPI-040, IMPI-007, IMPI-020, IMPI-032, IMPI-023, IMPI-039, IMPI-001, IMPI-019, IMPI-010, IMPI-008, IMPI-031, IMPI-057, IMPI-022, IMPI-035, IMPI-067 or IMPI-072.

B87. An anti-SARS-CoV-2 antibody, wherein the antibody comprises a variable heavy (VH) domain sequence and a variable light (VL) domain sequence and wherein the variable heavy (VH) domain and variable light (VL) domain sequences respectively comprise the variable heavy (VH) and variable light (VL) domain sequences of antibody IMPI-030, IMPI-053, IMPI-025, IMPI-040, IMPI-007, IMPI-020, IMPI-032, IMPI-023, IMPI-039, IMPI-001, IMPI-019, IMPI-010, IMPI-008, IMPI-031, IMPI-057, IMPI-022, IMPI-035, IMPI-067 or IMPI-072.

B88. The antibody according to clause B86 or B87, wherein the variable heavy (VH) domain sequence comprises the VH domain sequence of antibody IMPI-030 and the variable light (VL) domain sequence comprises the VL domain sequence of antibody IMPI-030.

B89. The antibody according to clause B86 or B87, wherein the variable heavy (VH) domain sequence comprises the VH domain sequence of antibody IMPI-053 and the variable light (VL) domain sequence comprises the VL domain sequence of antibody IMPI-053.

B90. The antibody according to clause B86 or B87, wherein the variable heavy (VH) domain sequence comprises the VH domain sequence of antibody IMPI-025 and the variable light (VL) domain sequence comprises the VL domain sequence of antibody IMPI-025.

B91. The antibody according to clause B86 or B87, wherein the variable heavy (VH) domain sequence comprises the VH domain sequence of antibody IMPI-040 and the variable light (VL) domain sequence comprises the VL domain sequence of antibody IMPI-040.

B92. The antibody according to clause B86 or B87, wherein the variable heavy (VH) domain sequence comprises the VH domain sequence of antibody IMPI-007 and the variable light (VL) domain sequence comprises the VL domain sequence of antibody IMPI-007.

B93. The antibody according to clause B86 or B87, wherein the variable heavy (VH) domain sequence comprises the VH domain sequence of antibody IMPI-020 and the variable light (VL) domain sequence comprises the VL domain sequence of antibody IMPI-020.

B94. The antibody according to clause B86 or B87, wherein the variable heavy (VH) domain sequence comprises the VH domain sequence of antibody IMPI-032 and the variable light (VL) domain sequence comprises the VL domain sequence of antibody IMPI-032.

B95. The antibody according to clause B86 or B87, wherein the variable heavy (VH) domain sequence comprises the VH domain sequence of antibody IMPI-023 and the variable light (VL) domain sequence comprises the VL domain sequence of antibody IMPI-023.

B96. The antibody according to clause B86 or B87, wherein the variable heavy (VH) domain sequence comprises the VH domain sequence of antibody IMPI-039 and the variable light (VL) domain sequence comprises the VL domain sequence of antibody IMPI-039.

B97. The antibody according to clause B86 or B87, wherein the variable heavy (VH) domain sequence comprises the VH domain sequence of antibody IMPI-001 and the variable light (VL) domain sequence comprises the VL domain sequence of antibody IMPI-001.

B98. The antibody according to clause B86 or B87, wherein the variable heavy (VH) domain sequence comprises the VH domain sequence of antibody IMPI-019 and the variable light (VL) domain sequence comprises the VL domain sequence of antibody IMPI-019.

B99. The antibody according to clause B86 or B87, wherein the variable heavy (VH) domain sequence comprises the VH domain sequence of antibody IMPI-010 and the variable light (VL) domain sequence comprises the VL domain sequence of antibody IMPI-010.

B100. The antibody according to clause B86 or B87, wherein the variable heavy (VH) domain sequence comprises the VH domain sequence of antibody IMPI-008 and the variable light (VL) domain sequence comprises the VL domain sequence of antibody IMPI-008.

B101. The antibody according to clause B86 or B87, wherein the variable heavy (VH) domain sequence comprises the VH domain sequence of antibody IMPI-031 and the variable light (VL) domain sequence comprises the VL domain sequence of antibody IMPI-031.

B102. The antibody according to clause B86 or B87, wherein the variable heavy (VH) domain sequence comprises the VH domain sequence of antibody IMPI-057 and the variable light (VL) domain sequence comprises the VL domain sequence of antibody IMPI-057.

B103. The antibody according to clause B86 or B87, wherein the variable heavy (VH) domain sequence comprises the VH domain sequence of antibody IMPI-022 and the variable light (VL) domain sequence comprises the VL domain sequence of antibody IMPI-022.

B104. The antibody according to clause B86 or B87, wherein the variable heavy (VH) domain sequence comprises the VH domain sequence of antibody IMPI-035 and the variable light (VL) domain sequence comprises the VL domain sequence of antibody IMPI-035.

B105. The antibody according to clause B86 or B87, wherein the variable heavy (VH) domain sequence comprises the VH domain sequence of antibody IMPI-067 and the variable light (VL) domain sequence comprises the VL domain sequence of antibody IMPI-067.

B106. The antibody according to clause B86 or B87, wherein the variable heavy (VH) domain sequence comprises the VH domain sequence of antibody IMPI-072 and the variable light (VL) domain sequence comprises the VL domain sequence of antibody IMPI-072.

B107. The antibody according to any one of clauses B1 to B4, comprising VH and/or VL domain framework regions of human germline gene segment sequences.

B108. The antibody according to any one of clauses B1 to B4 or B107, comprising an antibody VH domain which
i) is derived from recombination of a human heavy chain V gene segment, a human heavy chain D gene segment and a human heavy chain J gene segment, wherein
   the V gene segment is IGHV4-4*02, IGHV3-9*01 or IGHV3-30*18;
   and/or
   the J gene segment is IGHJ4*02 or IGHJ6*02, or
ii) comprises framework regions FR1, FR2, FR3 and FR4, wherein
   FR1 aligns with human germline V gene segment IGHV4-4*02, IGHV3-9*01 or IGHV3-30*18 with up to 1, 2, 3, 4, or 5 amino acid alterations,
   FR2 aligns with human germline V gene segment IGHV4-4*02, IGHV3-9*01 or IGHV3-30*18 with up to 1, 2, 3, 4, or 5 amino acid alterations,
   FR3 aligns with human germline V gene segment IGHV4-4*02, IGHV3-9*01 or IGHV3-30*18 with up to 1, 2, 3, 4 or 5 amino acid alterations, and/or
   FR4 aligns with human germline J gene segment IGHJ4*02 or IGHJ6*02 with up to 1, 2, 3, 4 or 5 amino acid alterations.

B109. The antibody according to anyone of clauses B1 to B4, B107 or B108, comprising an antibody VH domain which
i) is derived from recombination of a human heavy chain V gene segment IGHV4-4*02, IGHV3-9*01 or IGHV3-30*18, a human heavy chain D gene segment and a human heavy chain J gene segment, or
ii) comprises framework regions FR1, FR2, FR3 and FR4, wherein FR1, FR2 and FR3 each align with human germline V segment IGHV4-4*02, IGHV3-9*01 or IGHV3-30*18 with up to 1, 2, 3, 4 or 5 amino acid alterations.

B110. The antibody according to any one of clauses B1 to B4 or B107 to B109, wherein the J gene segment is IGHJ4*02 or IGHJ6*02, or wherein the VH domain framework region FR4 aligns with human germline J gene segment IGHJ4*02 or IGHJ6*02 with 1, 2, 3, 4 or 5 amino acid alterations.

B111. The antibody according to any one of clauses B1 to B4 or B107, comprising an antibody VL domain which
i) is derived from recombination of a human light chain V gene segment and a human light chain J gene segment, wherein
   the V gene segment is IGKV2D-30*01, IGKVID-13*d01 or IGKV3-20*01, and/or
   the J gene segment is IGKJ4*01 or
ii) comprises framework regions FR1, FR2, FR3 and FR4, wherein
   FR1 aligns with human germline V gene segment IGKV2D-30*01, IGKVID-13*d01 or IGKV3-20*01 with up to 1, 2, 3, 4, or 5 amino acid alterations,
   FR2 aligns with human germline V gene segment IGKV2D-30*01, IGKVID-13*d01 or IGKV3-20*01 with up to 1, 2, 3, 4, or 5 amino acid alterations
   FR3 aligns with human germline V gene segment IGKV2D-30*01, IGKVID-13*d01 or IGKV3-20*01 with up to 1, 2, 3, 4 or 5 amino acid alterations, and/or
   FR4 aligns with human germline J gene segment IGKJ4*01 with up to 1, 2, 3, 4 or 5 amino acid alterations.

B112. The antibody according to any one of clauses B1 to B4 or B107, comprising an antibody VL domain derived from recombination of a human light chain V gene segment and a human light chain J gene segment, wherein:
the V gene segment is IGKV2D-30*01, IGKVID-13*d01 or IGKV3-20*01, and optionally
the J gene segment is IGKJ4*01.

C1. A neutralising antibody that specifically binds to the S2 subunit of the SARS-CoV-2 spike protein.

C2. An antibody according to clause C1,
wherein the antibody comprises a variable heavy (VH) domain sequence comprising complementarity determining regions (CDRs) HCDR1, HCDR2 and HCDR3, and a variable light (VL) domain sequence comprising complementarity determining regions LCDR1, LCDR2 and LCDR3,
wherein the HCDR3 is the HCDR3 of antibody IMPI-003, IMPI-013, IMPI-063, IMPI-061, IMPI-062, IMPI-064, IMPI-065, IMPI-066, IMPI-069, IMPI-070 or IMPI-071.

C3. The antibody according to clause C2, wherein the HCDR3 is the HCDR3 of antibody IMPI-003.

C4. The antibody according to clause C2, wherein the HCDR3 is the HCDR3 of antibody IMPI-013.

C5. The antibody according to clause C2, wherein the HCDR3 is the HCDR3 of antibody IMPI-063.

C6. The antibody according to clause C2, wherein the HCDR3 is the HCDR3 of antibody IMPI-061.

C7. The antibody according to clause C2, wherein the HCDR3 is the HCDR3 of antibody IMPI-062.

C8. The antibody according to clause C2, wherein the HCDR3 is the HCDR3 of antibody IMPI-064.

C9. The antibody according to clause C2, wherein the HCDR3 is the HCDR3 of antibody IMPI-065.

C10. The antibody according to clause C2, wherein the HCDR3 is the HCDR3 of antibody IMPI-066.

C11. The antibody according to clause C2, wherein the HCDR3 is the HCDR3 of antibody IMPI-069.

C12. The antibody according to clause C2, wherein the HCDR3 is the HCDR3 of antibody IMPI-070.

C13. The antibody according to clause C2, wherein the HCDR3 is the HCDR3 of antibody IMPI-071.

C14. An anti-SARS-CoV-2 antibody,
wherein the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3,
wherein the CDRs are those of antibody IMPI-003, IMPI-013, IMPI-063, IMPI-061, IMPI-062, IMPI-064, IMPI-065, IMPI-066, IMPI-069, IMPI-070 or IMPI-071.

C15. An antibody according to clause C1,
wherein the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3,
wherein the CDRs are those of antibody IMPI-003, IMPI-013, IMPI-063, IMPI-061, IMPI-062, IMPI-064, IMPI-065, IMPI-066, IMPI-069, IMPI-070 or IMPI-071.

C16. The antibody according to clause C14 or clause C15, wherein the antibody has the CDRs of antibody IMPI-003.

C17. The antibody according to clause C14 or clause C15, wherein the antibody has the CDRs of antibody IMPI-013.

C18. The antibody according to clause C14 or clause C15, wherein the antibody has the CDRs of antibody IMPI-063.

C19. The antibody according to clause C14 or clause C15, wherein the antibody has the CDRs of antibody IMPI-061.

C20. The antibody according to clause C14 or clause C15, wherein the antibody has the CDRs of antibody IMPI-062.

C21. The antibody according to clause C14 or clause C15, wherein the antibody has the CDRs of antibody IMPI-064.

C22. The antibody according to clause C14 or clause C15, wherein the antibody has the CDRs of antibody IMPI-065.

C23. The antibody according to clause C14 or clause C15, wherein the antibody has the CDRs of antibody IMPI-066.

C24. The antibody according to clause C14 or clause C15, wherein the antibody has the CDRs of antibody IMPI-069.

C25. The antibody according to clause C14 or clause C15, wherein the antibody has the CDRs of antibody IMPI-070.

C26. The antibody according to clause C14 or clause C15, wherein the antibody has the CDRs of antibody IMPI-071.

C27. An antibody according to clause C1,
wherein the antibody comprises a variable heavy (VH) domain sequence and a variable light (VL) domain sequence and wherein the variable heavy (VH) domain and variable light (VL) domain sequences respectively comprise the variable heavy (VH) domain and variable light (VL) domain sequences of antibody IMPI-003, IMPI-013, IMPI-063, IMPI-061, IMPI-062, IMPI-064, IMPI-065, IMPI-066, IMPI-069, IMPI-070 or IMPI-071, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

C28. The antibody according to clause C27, wherein the antibody comprises a variable heavy (VH) domain sequence of antibody IMPI-003, optionally with 1, 2, 3, 4 or 5 amino acid alterations outside the complementarity determining regions (CDRs), and a variable light (VL) domain sequence of antibody IMPI-003, optionally with 1, 2, 3, 4 or 5 amino acid alterations outside the complementarity determining regions (CDRs).

C29. The antibody according to clause C27, wherein the antibody comprises a variable heavy (VH) domain sequence of antibody IMPI-013, optionally with 1, 2, 3, 4 or 5 amino acid alterations outside the complementarity determining regions (CDRs), and a variable light (VL) domain sequence of antibody IMPI-013, optionally with 1, 2, 3, 4 or 5 amino acid alterations outside the complementarity determining regions (CDRs).

C30. The antibody according to clause C27, wherein the antibody comprises a variable heavy (VH) domain sequence of antibody IMPI-063, optionally with 1, 2, 3, 4 or 5 amino acid alterations outside the complementarity determining regions (CDRs), and a variable light (VL) domain sequence of antibody IMPI-063, optionally with 1, 2, 3, 4 or 5 amino acid alterations outside the complementarity determining regions (CDRs).

C31. The antibody according to clause C27, wherein the antibody comprises a variable heavy (VH) domain sequence of antibody IMPI-061, optionally with 1, 2, 3, 4 or 5 amino acid alterations outside the complementarity determining regions (CDRs), and a variable light (VL) domain sequence of antibody IMPI-061, optionally with 1, 2, 3, 4 or 5 amino acid alterations outside the complementarity determining regions (CDRs).

C32. The antibody according to clause C27, wherein the antibody comprises a variable heavy (VH) domain sequence of antibody IMPI-062, optionally with 1, 2, 3, 4 or 5 amino acid alterations outside the complementarity determining regions (CDRs), and a variable light (VL) domain sequence of antibody IMPI-062, optionally with 1, 2, 3, 4 or 5 amino acid alterations outside the complementarity determining regions (CDRs).

C33. The antibody according to clause C27, wherein the antibody comprises a variable heavy (VH) domain sequence of antibody IMPI-064, optionally with 1, 2, 3, 4 or 5 amino acid alterations outside the complementarity determining regions (CDRs), and a variable light (VL) domain sequence of antibody IMPI-064, optionally with 1, 2, 3, 4 or 5 amino acid alterations outside the complementarity determining regions (CDRs).

C34. The antibody according to clause C27, wherein the antibody comprises a variable heavy (VH) domain sequence of antibody IMPI-065, optionally with 1, 2, 3, 4 or 5 amino acid alterations outside the complementarity determining regions (CDRs), and a variable light (VL) domain sequence of antibody IMPI-065, optionally with 1, 2, 3, 4 or 5 amino acid alterations outside the complementarity determining regions (CDRs).

C35. The antibody according to clause C27, wherein the antibody comprises a variable heavy (VH) domain sequence of antibody IMPI-066, optionally with 1, 2, 3, 4 or 5 amino acid alterations outside the complementarity determining regions (CDRs), and a variable light (VL) domain sequence of antibody IMPI-066, optionally with 1, 2, 3, 4 or 5 amino acid alterations outside the complementarity determining regions (CDRs).

C36. The antibody according to clause C27, wherein the antibody comprises a variable heavy (VH) domain sequence of antibody IMPI-069, optionally with 1, 2, 3, 4 or 5 amino acid alterations outside the complementarity determining regions (CDRs), and a variable light (VL) domain sequence of antibody IMPI-069, optionally with 1, 2, 3, 4 or 5 amino acid alterations outside the complementarity determining regions (CDRs).

C37. The antibody according to clause C27, wherein the antibody comprises a variable heavy (VH) domain sequence of antibody IMPI-070, optionally with 1, 2, 3, 4 or 5 amino acid alterations outside the complementarity determining regions (CDRs), and a variable light (VL) domain sequence of antibody IMPI-070, optionally with 1, 2, 3, 4 or 5 amino acid alterations outside the complementarity determining regions (CDRs).

C38. The antibody according to clause C27, wherein the antibody comprises a variable heavy (VH) domain sequence of antibody IMPI-071, optionally with 1, 2, 3, 4 or 5 amino acid alterations outside the complementarity determining regions (CDRs), and a variable light (VL) domain sequence of antibody IMPI-071, optionally with 1, 2, 3, 4 or 5 amino acid alterations outside the complementarity determining regions (CDRs).

C39. An antibody according to clause C1, wherein the antibody comprises a variable heavy (VH) domain sequence and a variable light (VL) domain sequence and wherein the variable heavy (VH) domain and variable light (VL) domain sequences respectively comprise a sequence having at least 90% identity to the variable heavy (VH) and variable light (VL) domain sequences of antibody IMPI-003, IMPI-013, IMPI-063, IMPI-061, IMPI-062, IMPI-064, IMPI-065, IMPI-066, IMPI-069, IMPI-070 or IMPI-071, provided that the antibody has the CDRs of antibody IMPI-003, IMPI-013, IMPI-063, IMPI-061, IMPI-062, IMPI-064, IMPI-065, IMPI-066, IMPI-069, IMPI-070 or IMPI-071.

C40. The antibody according to clause C39, wherein the variable heavy (VH) domain sequence comprises a sequence having at least 90% identity to the VH domain sequence of antibody IMPI-003 and the variable light (VL) domain sequence comprises a sequence having at least 90% identity to the VL domain sequence of antibody IMPI-003, provided that the antibody has the CDRs of antibody IMPI-003.

C41. The antibody according to clause C39, wherein the variable heavy (VH) domain sequence comprises a sequence having at least 90% identity to the VH domain sequence of antibody IMPI-013 and the variable light (VL) domain sequence comprises a sequence having at least 90% identity to the VL domain sequence of antibody IMPI-013, provided that the antibody has the CDRs of antibody IMPI-013.

C42. The antibody according to clause C39, wherein the variable heavy (VH) domain sequence comprises a sequence having at least 90% identity to the VH domain sequence of antibody IMPI-063 and the variable light (VL) domain sequence comprises a sequence having at least 90% identity to the VL domain sequence of antibody IMPI-063, provided that the antibody has the CDRs of antibody IMPI-063.

C43. The antibody according to clause C39, wherein the variable heavy (VH) domain sequence comprises a sequence having at least 90% identity to the VH domain sequence of antibody IMPI-061 and the variable light (VL) domain sequence comprises a sequence having at least 90% identity to the VL domain sequence of antibody IMPI-061, provided that the antibody has the CDRs of antibody IMPI-061.

C44. The antibody according to clause C39, wherein the variable heavy (VH) domain sequence comprises a sequence having at least 90% identity to the VH domain sequence of antibody IMPI-062 and the variable light (VL) domain sequence comprises a sequence having at least 90% identity to the VL domain sequence of antibody IMPI-062, provided that the antibody has the CDRs of antibody IMPI-062.

C45. The antibody according to clause C39, wherein the variable heavy (VH) domain sequence comprises a sequence having at least 90% identity to the VH domain sequence of antibody IMPI-064 and the variable light (VL) domain sequence comprises a sequence having at least 90% identity to the VL domain sequence of antibody IMPI-064, provided that the antibody has the CDRs of antibody IMPI-064.

C46. The antibody according to clause C39, wherein the variable heavy (VH) domain sequence comprises a sequence having at least 90% identity to the VH domain sequence of antibody IMPI-065 and the variable light (VL) domain sequence comprises a sequence having at least 90% identity to the VL domain sequence of antibody IMPI-065, provided that the antibody has the CDRs of antibody IMPI-065.

C47. The antibody according to clause C39, wherein the variable heavy (VH) domain sequence comprises a sequence having at least 90% identity to the VH domain sequence of antibody IMPI-066 and the variable light (VL) domain sequence comprises a sequence having at least 90% identity to the VL domain sequence of antibody IMPI-066, provided that the antibody has the CDRs of antibody IMPI-066.

C48. The antibody according to clause C39, wherein the variable heavy (VH) domain sequence comprises a sequence having at least 90% identity to the VH domain sequence of antibody IMPI-069 and the variable light (VL) domain sequence comprises a sequence having at least 90% identity to the VL domain sequence of antibody IMPI-069, provided that the antibody has the CDRs of antibody IMPI-069.

C49. The antibody according to clause C39, wherein the variable heavy (VH) domain sequence comprises a sequence having at least 90% identity to the VH domain sequence of antibody IMPI-070 and the variable light (VL) domain sequence comprises a sequence having at least 90% identity to the VL domain sequence of antibody IMPI-070, provided that the antibody has the CDRs of antibody IMPI-070.

C50. The antibody according to clause C39, wherein the variable heavy (VH) domain sequence comprises a sequence having at least 90% identity to the VH domain sequence of antibody IMPI-071 and the variable light (VL) domain sequence comprises a sequence having at least 90% identity to the VL domain sequence of antibody IMPI-071, provided that the antibody has the CDRs of antibody IMPI-071.

C51. An antibody according to clause C1, wherein the antibody comprises a variable heavy (VH) domain sequence and a variable light (VL) domain sequence and wherein the variable heavy (VH) domain and variable light (VL) domain sequences respectively comprise the variable heavy (VH) and variable light (VL) domain sequences of antibody IMPI-003, IMPI-013, IMPI-063, IMPI-061, IMPI-062, IMPI-064, IMPI-065, IMPI-066, IMPI-069, IMPI-070 or IMPI-071.

C52. An anti-SARS-CoV-2 antibody, wherein the antibody comprises a variable heavy (VH) domain sequence and a variable light (VL) domain sequence and wherein the variable heavy (VH) domain and variable light (VL) domain sequences respectively comprise the variable heavy (VH) and variable light (VL) domain sequences of antibody IMPI-003, IMPI-013, IMPI-063, IMPI-061, IMPI-062, IMPI-064, IMPI-065, IMPI-066, IMPI-069, IMPI-070 or IMPI-071.

C53. The antibody according to clause C51 or C52, wherein the variable heavy (VH) domain sequence comprises the VH domain sequence of antibody IMPI-003 and the variable light (VL) domain sequence comprises the VL domain sequence of antibody IMPI-003.

C54. The antibody according to clause C51 or C52, wherein the variable heavy (VH) domain sequence comprises the VH domain sequence of antibody IMPI-013 and the variable light (VL) domain sequence comprises the VL domain sequence of antibody IMPI-013.

C55. The antibody according to clause C51 or C52, wherein the variable heavy (VH) domain sequence comprises the VH domain sequence of antibody IMPI-063 and the variable light (VL) domain sequence comprises the VL domain sequence of antibody IMPI-063.

C56. The antibody according to clause C51 or C52, wherein the variable heavy (VH) domain sequence comprises the VH domain sequence of antibody IMPI-061 and the variable light (VL) domain sequence comprises the VL domain sequence of antibody IMPI-061.

C57. The antibody according to clause C51 or C52, wherein the variable heavy (VH) domain sequence comprises the VH domain sequence of antibody IMPI-062 and the variable light (VL) domain sequence comprises the VL domain sequence of antibody IMPI-062.

C58. The antibody according to clause C51 or C52, wherein the variable heavy (VH) domain sequence comprises the VH domain sequence of antibody IMPI-064 and the variable light (VL) domain sequence comprises the VL domain sequence of antibody IMPI-064.

C59. The antibody according to clause C51 or C52, wherein the variable heavy (VH) domain sequence comprises the VH domain sequence of antibody IMPI-065 and the variable light (VL) domain sequence comprises the VL domain sequence of antibody IMPI-065.

C60. The antibody according to clause C51 or C52, wherein the variable heavy (VH) domain sequence comprises the VH domain sequence of antibody IMPI-066 and the variable light (VL) domain sequence comprises the VL domain sequence of antibody IMPI-066.

C61. The antibody according to clause C51 or C52, wherein the variable heavy (VH) domain sequence comprises the VH domain sequence of antibody IMPI-069 and the variable light (VL) domain sequence comprises the VL domain sequence of antibody IMPI-069.

C62. The antibody according to clause C51 or C52, wherein the variable heavy (VH) domain sequence comprises the VH domain sequence of antibody IMPI-070 and the variable light (VL) domain sequence comprises the VL domain sequence of antibody IMPI-070.

C63. The antibody according to clause C51 or C52, wherein the variable heavy (VH) domain sequence comprises the VH domain sequence of antibody IMPI-071 and the variable light (VL) domain sequence comprises the VL domain sequence of antibody IMPI-071.

C64. The antibody according to clause C1, comprising VH and/or VL domain framework regions of human germline gene segment sequences.

C65. The antibody according to clause C1 or C64, comprising an antibody VH domain which
i) is derived from recombination of a human heavy chain V gene segment, a human heavy chain D gene segment and a human heavy chain J gene segment, wherein
   the V gene segment is IGHV3-9*01 or IGHV3-20*d01;
   and/or
   the J gene segment is IGHJ6*02 or IGHJ4*02, or
ii) comprises framework regions FR1, FR2, FR3 and FR4, wherein
   FR1 aligns with human germline V gene segment IGHV3-9*01 or IGHV3-20*d01 with up to 1, 2, 3, 4, or 5 amino acid alterations,
   FR2 aligns with human germline V gene segment IGHV3-9*01 or IGHV3-20*d01 with up to 1, 2, 3, 4, or 5 amino acid alterations,
   FR3 aligns with human germline V gene segment IGHV3-9*01 or IGHV3-20*d01 with up to 1, 2, 3, 4 or 5 amino acid alterations, and/or
   FR4 aligns with human germline J gene segment IGHJ6*02 or IGHJ4*02 with up to 1, 2, 3, 4 or 5 amino acid alterations.

C66. The antibody according to any one of clauses C1, C64 or C65, comprising an antibody VH domain which
i) is derived from recombination of a human heavy chain V gene segment IGHV3-9*01 or IGHV3-20*d01, a human heavy chain D gene segment and a human heavy chain J gene segment, or
ii) comprises framework regions FR1, FR2, FR3 and FR4, wherein FR1, FR2 and FR3 each align with human germline V segment IGHV3-9*01 or IGHV3-20*d01 with up to 1, 2, 3, 4 or 5 amino acid alterations.

C67. The antibody according to any one of clauses C1 or C61 to C66, wherein the J gene segment is IGHJ6*02 or IGHJ4*02, or wherein the VH domain framework region FR4 aligns with human germline J gene segment IGHJ6*02 or IGHJ4*02 with 1, 2, 3, 4 or 5 amino acid alterations.

C68. The antibody according to clause C1 or C64, comprising an antibody VL domain which
i) is derived from recombination of a human light chain V gene segment and a human light chain J gene segment, wherein
   the V gene segment is IGKV1-6*01 or IGKV3-20*01, and/or
   the J gene segment is IGKJ1*01 or IGKJ2*04; or
ii) comprises framework regions FR1, FR2, FR3 and FR4, wherein
   FR1 aligns with human germline V gene segment IGKV1-6*01 or IGKV3-20*01 with up to 1, 2, 3, 4, or 5 amino acid alterations,
   FR2 aligns with human germline V gene segment IGKV1-6*01 or IGKV3-20*01 with up to 1, 2, 3, 4, or 5 amino acid alterations
   FR3 aligns with human germline V gene segment IGKV1-6*01 or IGKV3-20*01 with up to 1, 2, 3, 4 or 5 amino acid alterations, and/or
   FR4 aligns with human germline J gene segment IGKJ1*01 or IGKJ2*04 with up to 1, 2, 3, 4 or 5 amino acid alterations.

C69. The antibody according to any one of clause C1 or C64, comprising an antibody VL domain derived from recombination of a human light chain V gene segment and a human light chain J gene segment,
wherein:
   the V gene segment is IGKV1-6*01 or IGKV3-20*01, and optionally
   the J gene segment is IGKJ1*01 or IGKJ2*04.

D1. An antibody that specifically binds to the receptor binding domain (RBD) of the SARS-CoV-2 spike protein, wherein the antibody does not compete for binding to the SARS-CoV-2 spike protein with the human ACE2 receptor.

D2. An antibody according to clause D1, wherein the antibody is a neutralising antibody.

D3. An antibody according to clause D1 or clause D2, wherein the antibody increases binding between SARS-CoV-2 and the human ACE2 receptor.

D4. An antibody according to any one of clauses D1 to D3,
wherein the antibody comprises a variable heavy (VH) domain sequence comprising complementarity determining regions (CDRs) HCDR1, HCDR2 and HCDR3, and a variable light (VL) domain sequence comprising complementarity determining regions LCDR1, LCDR2 and LCDR3,
wherein the HCDR3 is the HCDR3 of antibody IMPI-026, IMPI-034, IMPI-016, IMPI-050, IMPI-049, IMPI-015, IMPI-009, IMPI-011, IMPI-044, IMPI-046, IMPI-051, IMPI-024, IMPI-058, IMPI-043, IMPI-045, IMPI-027, IMPI-018, IMPI-048, IMPI-033, IMPI-014, IMPI-038 or IMPI-068.

D5. An antibody according to clause D2,
wherein the antibody comprises a variable heavy (VH) domain sequence comprising complementarity determining regions (CDRs) HCDR1, HCDR2 and HCDR3, and a variable light (VL) domain sequence comprising complementarity determining regions LCDR1, LCDR2 and LCDR3, wherein the HCDR3 is the HCDR3 of antibody IMPI-024, IMPI-027, IMPI-038 or IMPI-068.

D6. An antibody according to clause D3, wherein the antibody comprises a variable heavy (VH) domain sequence comprising complementarity determining regions (CDRs) HCDR1, HCDR2 and HCDR3, and a variable light (VL) domain sequence comprising complementarity determining regions LCDR1, LCDR2 and LCDR3, wherein the HCDR3 is the HCDR3 of antibody IMPI-027, IMPI-033, IMPI-038 or IMPI-068.

D7. The antibody according to clause D4, wherein the HCDR3 is the HCDR3 of antibody IMPI-026.

D8. The antibody according to clause D4, wherein the HCDR3 is the HCDR3 of antibody IMPI-034.

D9. The antibody according to clause D4, wherein the HCDR3 is the HCDR3 of antibody IMPI-016.

D10. The antibody according to clause D4, wherein the HCDR3 is the HCDR3 of antibody IMPI-050.

D1I. The antibody according to clause D4, wherein the HCDR3 is the HCDR3 of antibody IMPI-049.

D12. The antibody according to clause D4, wherein the HCDR3 is the HCDR3 of antibody IMPI-015.

D13. The antibody according to clause D4, wherein the HCDR3 is the HCDR3 of antibody IMPI-009.

D14. The antibody according to clause D4, wherein the HCDR3 is the HCDR3 of antibody IMPI-011.

D15. The antibody according to clause D4, wherein the HCDR3 is the HCDR3 of antibody IMPI-044.

D16. The antibody according to clause D4, wherein the HCDR3 is the HCDR3 of antibody IMPI-046.

D17. The antibody according to clause D4, wherein the HCDR3 is the HCDR3 of antibody IMPI-051.

D18. The antibody according to clause D4, wherein the HCDR3 is the HCDR3 of antibody IMPI-024.

D19. The antibody according to clause D4, wherein the HCDR3 is the HCDR3 of antibody IMPI-058.

D20. The antibody according to clause D4, wherein the HCDR3 is the HCDR3 of antibody IMPI-043.

D21. The antibody according to clause D4, wherein the HCDR3 is the HCDR3 of antibody IMPI-045.

D22. The antibody according to clause D4, wherein the HCDR3 is the HCDR3 of antibody IMPI-027.

D23. The antibody according to clause D4, wherein the HCDR3 is the HCDR3 of antibody IMPI-018.

D24. The antibody according to clause D4, wherein the HCDR3 is the HCDR3 of antibody IMPI-048.

D25. The antibody according to clause D4, wherein the HCDR3 is the HCDR3 of antibody IMPI-033.

D26. The antibody according to clause D4, wherein the HCDR3 is the HCDR3 of antibody IMPI-014.

D27. The antibody according to clause D4, wherein the HCDR3 is the HCDR3 of antibody IMPI-038.

D28. The antibody according to clause D4, wherein the HCDR3 is the HCDR3 of antibody IMPI-068.

D29. An anti-SARS-CoV-2 antibody, wherein the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3, wherein the CDRs are those of antibody IMPI-026, IMPI-034, IMPI-016, IMPI-050, IMPI-049, IMPI-015, IMPI-009, IMPI-011, IMPI-044, IMPI-046, IMPI-051, IMPI-024, IMPI-058, IMPI-043, IMPI-045, IMPI-027, IMPI-018, IMPI-048, IMPI-033, IMPI-014, IMPI-038 or IMPI-068.

D30. An antibody according to any one of clauses D1 to D3, wherein the antibody comprises a variable heavy (VH) domain sequence comprising complementarity determining regions (CDRs) HCDR1, HCDR2 and HCDR3, and a variable light (VL) domain sequence comprising complementarity determining regions LCDR1, LCDR2 and LCDR3, wherein the CDRs are those of antibody IMPI-026, IMPI-034, IMPI-016, IMPI-050, IMPI-049, IMPI-015, IMPI-009, IMPI-011, IMPI-044, IMPI-046, IMPI-051, IMPI-024, IMPI-058, IMPI-043, IMPI-045, IMPI-027, IMPI-018, IMPI-048, IMPI-033, IMPI-014, IMPI-038 or IMPI-068.

D31. An antibody according to clause D2, wherein the antibody comprises a variable heavy (VH) domain sequence comprising complementarity determining regions (CDRs) HCDR1, HCDR2 and HCDR3, and a variable light (VL) domain sequence comprising complementarity determining regions LCDR1, LCDR2 and LCDR3, wherein the CDRs are those of antibody IMPI-024, IMPI-027, IMPI-038 or IMPI-068.

D32. An antibody according to clause D3, wherein the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3, wherein the CDRs are those of antibody IMPI-027, IMPI-033, IMPI-038 or IMPI-068.

D33. The antibody according to clause D29 or clause D30, wherein the antibody has the CDRs of antibody IMPI-026.

D34. The antibody according to clause D29 or clause D30, wherein the antibody has the CDRs of antibody IMPI-034.

D35. The antibody according to clause D29 or clause D30, wherein the antibody has the CDRs of antibody IMPI-016.

D36. The antibody according to clause D29 or clause D30, wherein the antibody has the CDRs of antibody IMPI-050.

D37. The antibody according to clause D29 or clause D30, wherein the antibody has the CDRs of antibody IMPI-049.

D38. The antibody according to clause D29 or clause D30, wherein the antibody has the CDRs of antibody IMPI-015.

D39. The antibody according to clause D29 or clause D30, wherein the antibody has the CDRs of antibody IMPI-009.

D40. The antibody according to clause D29 or clause D30, wherein the antibody has the CDRs of antibody IMPI-011.

D41. The antibody according to clause D29 or clause D30, wherein the antibody has the CDRs of antibody IMPI-044.

D42. The antibody according to clause D29 or clause D30, wherein the antibody has the CDRs of antibody IMPI-046.

D43. The antibody according to clause D29 or clause D30, wherein the antibody has the CDRs of antibody IMPI-051.

D44. The antibody according to clause D29 or clause D30, wherein the antibody has the CDRs of antibody IMPI-024.

D45. The antibody according to clause D29 or clause D30, wherein the antibody has the CDRs of antibody IMPI-058.

D46. The antibody according to clause D29 or clause D30, wherein the antibody has the CDRs of antibody IMPI-043.

D47. The antibody according to clause D29 or clause D30, wherein the antibody has the CDRs of antibody IMPI-045.

D48. The antibody according to clause D29 or clause D30, wherein the antibody has the CDRs of antibody IMPI-027.

D49. The antibody according to clause D29 or clause D30, wherein the antibody has the CDRs of antibody IMPI-018.

D50. The antibody according to clause D29 or clause D30, wherein the antibody has the CDRs of antibody IMPI-048.

D51. The antibody according to clause D29 or clause D30, wherein the antibody has the CDRs of antibody IMPI-033.

D52. The antibody according to clause D29 or clause D30, wherein the antibody has the CDRs of antibody IMPI-014.

D53. The antibody according to clause D29 or clause D30, wherein the antibody has the CDRs of antibody IMPI-038.

D54. The antibody according to clause D29 or clause D30, wherein the antibody has the CDRs of antibody IMPI-068.

D55. An antibody according to any one of clauses D1 to D3, wherein the antibody comprises a variable heavy (VH) domain sequence and a variable light (VL) domain sequence and wherein the variable heavy (VH) domain and variable light (VL) domain sequences respectively comprise the variable heavy (VH) domain and variable light (VL) domain sequences of antibody IMPI-026, IMPI-034, IMPI-016, IMPI-050, IMPI-049, IMPI-015, IMPI-009, IMPI-011, IMPI-044, IMPI-046, IMPI-051, IMPI-024, IMPI-058, IMPI-043, IMPI-045, IMPI-027, IMPI-018, IMPI-048, IMPI-033, IMPI-014, IMPI-038 or IMPI-068, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

D56. An antibody according to clause D2, wherein the antibody comprises a variable heavy (VH) domain sequence and a variable light (VL) domain sequence and wherein the variable heavy (VH) domain and variable light (VL) domain sequences respectively comprise the variable heavy (VH) domain and variable light (VL) domain sequences of antibody IMPI-024, IMPI-027, IMPI-038 or IMPI-068, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

D57. An antibody according to clause D3, wherein the antibody comprises a variable heavy (VH) domain sequence and a variable light (VL) domain sequence and wherein the variable heavy (VH) domain and variable light (VL) domain sequences respectively comprise the variable heavy (VH) domain and variable light (VL) domain sequences of antibody IMPI-027, IMPI-033, IMPI-038 or IMPI-068, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

D58. The antibody according to clause D55, wherein the antibody comprises a variable heavy (VH) domain sequence of antibody IMPI-026, optionally with 1, 2, 3, 4 or 5 amino acid alterations outside the complementarity determining regions (CDRs), and a variable light (VL) domain sequence of antibody IMPI-026, optionally with 1, 2, 3, 4 or 5 amino acid alterations outside the complementarity determining regions (CDRs).

D59. The antibody according to clause D55, wherein the antibody comprises a variable heavy (VH) domain sequence of antibody IMPI-034, optionally with 1, 2, 3, 4 or 5 amino acid alterations outside the complementarity determining regions (CDRs), and a variable light (VL) domain sequence of antibody IMPI-034, optionally with 1, 2, 3, 4 or 5 amino acid alterations outside the complementarity determining regions (CDRs).

D60. The antibody according to clause D55, wherein the antibody comprises a variable heavy (VH) domain sequence of antibody IMPI-016, optionally with 1, 2, 3, 4 or 5 amino acid alterations outside the complementarity determining regions (CDRs), and a variable light (VL) domain sequence of antibody IMPI-016, optionally with 1, 2, 3, 4 or 5 amino acid alterations outside the complementarity determining regions (CDRs).

D61. The antibody according to clause D55, wherein the antibody comprises a variable heavy (VH) domain sequence of antibody IMPI-050, optionally with 1, 2, 3, 4 or 5 amino acid alterations outside the complementarity determining regions (CDRs), and a variable light (VL) domain sequence of antibody IMPI-050, optionally with 1, 2, 3, 4 or 5 amino acid alterations outside the complementarity determining regions (CDRs).

D62. The antibody according to clause D55, wherein the antibody comprises a variable heavy (VH) domain sequence of antibody IMPI-049, optionally with 1, 2, 3, 4 or 5 amino acid alterations outside the complementarity determining regions (CDRs), and a variable light (VL) domain sequence of antibody IMPI-049, optionally with 1, 2, 3, 4 or 5 amino acid alterations outside the complementarity determining regions (CDRs).

D63. The antibody according to clause D55, wherein the antibody comprises a variable heavy (VH) domain sequence of antibody IMPI-015, optionally with 1, 2, 3, 4 or 5 amino acid alterations outside the complementarity determining regions (CDRs), and a variable light (VL) domain sequence of antibody IMPI-015, optionally with 1, 2, 3, 4 or 5 amino acid alterations outside the complementarity determining regions (CDRs).

D64. The antibody according to clause D55, wherein the antibody comprises a variable heavy (VH) domain sequence of antibody IMPI-009, optionally with 1, 2, 3, 4 or 5 amino acid alterations outside the complementarity determining regions (CDRs), and a variable light (VL) domain sequence of antibody IMPI-009, optionally with 1, 2, 3, 4 or 5 amino acid alterations outside the complementarity determining regions (CDRs).

D65. The antibody according to clause D55, wherein the antibody comprises a variable heavy (VH) domain sequence of antibody IMPI-011, optionally with 1, 2, 3, 4 or 5 amino acid alterations outside the complementarity determining regions (CDRs), and a variable light (VL) domain sequence of antibody IMPI-011, optionally with 1, 2, 3, 4 or 5 amino acid alterations outside the complementarity determining regions (CDRs).

D66. The antibody according to clause D55, wherein the antibody comprises a variable heavy (VH) domain sequence of antibody IMPI-044, optionally with 1, 2, 3, 4 or 5 amino acid alterations outside the complementarity determining regions (CDRs), and a variable light (VL) domain sequence of antibody IMPI-044, optionally with 1, 2, 3, 4 or 5 amino acid alterations outside the complementarity determining regions (CDRs).

D67. The antibody according to clause D55, wherein the antibody comprises a variable heavy (VH) domain sequence of antibody IMPI-046, optionally with 1, 2, 3, 4 or 5 amino acid alterations outside the complementarity determining regions (CDRs), and a variable light (VL) domain sequence of antibody IMPI-046, optionally with 1, 2, 3, 4 or 5 amino acid alterations outside the complementarity determining regions (CDRs).

D68. The antibody according to clause D55, wherein the antibody comprises a variable heavy (VH) domain sequence of antibody IMPI-051, optionally with 1, 2, 3, 4 or 5 amino acid alterations outside the complementarity determining regions (CDRs), and a variable light (VL) domain sequence of antibody IMPI-051, optionally with 1, 2, 3, 4 or 5 amino acid alterations outside the complementarity determining regions (CDRs).

D69. The antibody according to clause D55, wherein the antibody comprises a variable heavy (VH) domain sequence of antibody IMPI-024, optionally with 1, 2, 3, 4 or 5 amino acid alterations outside the complementarity determining regions (CDRs), and a variable light (VL) domain sequence of antibody IMPI-024, optionally with 1, 2, 3, 4 or 5 amino acid alterations outside the complementarity determining regions (CDRs).

D70. The antibody according to clause D55, wherein the antibody comprises a variable heavy (VH) domain sequence of antibody IMPI-058, optionally with 1, 2, 3, 4 or 5 amino acid alterations outside the complementarity determining regions (CDRs), and a variable light (VL) domain sequence of antibody IMPI-058, optionally with 1, 2, 3, 4 or 5 amino acid alterations outside the complementarity determining regions (CDRs).

D71. The antibody according to clause D55, wherein the antibody comprises a variable heavy (VH) domain sequence of antibody IMPI-043, optionally with 1, 2, 3, 4 or 5 amino acid alterations outside the complementarity determining regions (CDRs), and a variable light (VL) domain sequence of antibody IMPI-043, optionally with 1, 2, 3, 4 or 5 amino acid alterations outside the complementarity determining regions (CDRs).

D72. The antibody according to clause D55, wherein the antibody comprises a variable heavy (VH) domain sequence of antibody IMPI-045, optionally with 1, 2, 3, 4 or 5 amino acid alterations outside the complementarity determining regions (CDRs), and a variable light (VL) domain sequence of antibody IMPI-045, optionally with 1, 2, 3, 4 or 5 amino acid alterations outside the complementarity determining regions (CDRs).

D73. The antibody according to clause D55, wherein the antibody comprises a variable heavy (VH) domain sequence of antibody IMPI-027, optionally with 1, 2, 3, 4 or 5 amino acid alterations outside the complementarity determining regions (CDRs), and a variable light (VL) domain sequence of antibody IMPI-027, optionally with 1, 2, 3, 4 or 5 amino acid alterations outside the complementarity determining regions (CDRs).

D74. The antibody according to clause D55, wherein the antibody comprises a variable heavy (VH) domain sequence of antibody IMPI-018, optionally with 1, 2, 3, 4 or 5 amino acid alterations outside the complementarity determining regions (CDRs), and a variable light (VL) domain sequence of antibody IMPI-018, optionally with 1, 2, 3, 4 or 5 amino acid alterations outside the complementarity determining regions (CDRs).

D75. The antibody according to clause D55, wherein the antibody comprises a variable heavy (VH) domain sequence of antibody IMPI-048, optionally with 1, 2, 3, 4 or 5 amino acid alterations outside the complementarity determining regions (CDRs), and a variable light (VL) domain sequence of antibody IMPI-048, optionally with 1, 2, 3, 4 or 5 amino acid alterations outside the complementarity determining regions (CDRs).

D76. The antibody according to clause D55, wherein the antibody comprises a variable heavy (VH) domain sequence of antibody IMPI-033, optionally with 1, 2, 3, 4 or 5 amino acid alterations outside the complementarity determining regions (CDRs), and a variable light (VL) domain sequence of antibody IMPI-033, optionally with 1, 2, 3, 4 or 5 amino acid alterations outside the complementarity determining regions (CDRs).

D77. The antibody according to clause D55, wherein the antibody comprises a variable heavy (VH) domain sequence of antibody IMPI-014, optionally with 1, 2, 3, 4 or 5 amino acid alterations outside the complementarity determining regions (CDRs), and a variable light (VL) domain sequence of antibody IMPI-014, optionally with 1, 2, 3, 4 or 5 amino acid alterations outside the complementarity determining regions (CDRs).

D78. The antibody according to clause D55, wherein the antibody comprises a variable heavy (VH) domain sequence of antibody IMPI-038, optionally with 1, 2, 3, 4 or 5 amino acid alterations outside the complementarity determining regions (CDRs), and a variable light (VL) domain sequence of antibody IMPI-038, optionally with 1, 2, 3, 4 or 5 amino acid alterations outside the complementarity determining regions (CDRs).

D79. The antibody according to clause D55, wherein the antibody comprises a variable heavy (VH) domain sequence of antibody IMPI-068, optionally with 1, 2, 3, 4 or 5 amino acid alterations outside the complementarity determining regions (CDRs), and a variable light (VL) domain sequence of antibody IMPI-068, optionally with 1, 2, 3, 4 or 5 amino acid alterations outside the complementarity determining regions (CDRs).

D80. An antibody according to any one of clauses D1 to D3, wherein the antibody comprises a variable heavy (VH) domain sequence and a variable light (VL) domain sequence and wherein the variable heavy (VH) domain and variable light (VL) domain sequences respectively comprise a sequence having at least 90% identity to the variable heavy (VH) and variable light (VL) domain sequences of antibody IMPI-026, IMPI-034, IMPI-016, IMPI-050, IMPI-049, IMPI-015, IMPI-009, IMPI-011, IMPI-044, IMPI-046, IMPI-051, IMPI-024, IMPI-058, IMPI-043, IMPI-045, IMPI-027, IMPI-018, IMPI-048, IMPI-033, IMPI-014, IMPI-038 or IMPI-068, provided that the antibody has the CDRs of antibody IMPI-026, IMPI-034, IMPI-016, IMPI-050, IMPI-049, IMPI-015, IMPI-009, IMPI-011, IMPI-044, IMPI-046, IMPI-051, IMPI-024, IMPI-058, IMPI-043, IMPI-045, IMPI-027, IMPI-018, IMPI-048, IMPI-033, IMPI-014, IMPI-038 or IMPI-068. D81. An antibody according to clause D2, wherein the antibody comprises a variable heavy (VH) domain sequence and a variable light (VL) domain sequence and wherein the variable heavy (VH) domain and variable light (VL) domain sequences respectively comprise a sequence having at least 90% identity to the variable heavy (VH) and variable light (VL) domain sequences of antibody IMPI-024, IMPI-027, IMPI-038 or IMPI-068, provided that the antibody has the CDRs of antibody IMPI-024, IMPI-027, IMPI-038 or IMPI-068.

D82. An antibody according to clause D3, wherein the antibody comprises a variable heavy (VH) domain sequence and a variable light (VL) domain sequence and wherein the variable heavy (VH) domain and variable light (VL) domain sequences respectively comprise a sequence having at least 90% identity to the variable heavy (VH) and variable light (VL) domain sequences of antibody IMPI-027, IMPI-033, IMPI-038 or IMPI-068, provided that the antibody has the CDRs of antibody IMPI-027, IMPI-033, IMPI-038 or IMPI-068. D83. The antibody according to clause D80, wherein the variable heavy (VH) domain sequence comprises a sequence having at least 90% identity to the VH domain sequence of antibody IMPI-026 and the variable light (VL) domain sequence comprises a sequence having at least 90% identity to the VL domain sequence of antibody IMPI-026, provided that the antibody has the CDRs of antibody IMPI-026. D84. The antibody according to clause D80, wherein the variable heavy (VH) domain sequence comprises a sequence having at least 90% identity to the VH domain sequence of antibody IMPI-034 and the variable light (VL) domain sequence comprises a sequence having at least 90% identity to the VL domain sequence of antibody IMPI-034, provided that the antibody has the CDRs of antibody IMPI-034. D85. The antibody according to clause D80, wherein the variable heavy (VH) domain sequence comprises a sequence having at least 90% identity to the VH domain sequence of antibody IMPI-016 and the variable light (VL) domain sequence comprises a sequence having at least 90% identity to the VL domain sequence of antibody IMPI-016, provided that the antibody has the CDRs of antibody IMPI-016. D86. The antibody according to clause D80, wherein the variable heavy (VH) domain sequence comprises a sequence having at least 90% identity to the VH domain sequence of antibody IMPI-050 and the variable light (VL) domain sequence comprises a sequence having at least 90% identity to the VL domain sequence of antibody IMPI-050, provided that the antibody has the CDRs of antibody IMPI-050. D87. The antibody according to clause D80, wherein the variable heavy (VH) domain sequence comprises a sequence having at least 90% identity to the VH domain sequence of antibody IMPI-049 and the variable light (VL) domain sequence comprises a sequence having at least 90% identity to the VL domain sequence of antibody IMPI-049, provided that the antibody has the CDRs of antibody IMPI-049. D88. The antibody according to clause D80, wherein the variable heavy (VH) domain sequence comprises a sequence having at least 90% identity to the VH domain sequence of antibody IMPI-015 and the variable light (VL) domain sequence comprises a sequence having at least 90% identity to the VL domain sequence of antibody IMPI-015, provided that the antibody has the CDRs of antibody IMPI-015. D89. The antibody according to clause D80, wherein the variable heavy (VH) domain sequence comprises a sequence having at least 90% identity to the VH domain sequence of antibody IMPI-009 and the variable light (VL) domain sequence comprises a sequence having at least 90% identity to the VL domain sequence of antibody IMPI-009, provided that the antibody has the CDRs of antibody IMPI-009. D90. The antibody according to clause D80, wherein the variable heavy (VH) domain sequence comprises a sequence having at least 90% identity to the VH domain sequence of antibody IMPI-011 and the variable light (VL) domain sequence comprises a sequence having at least 90% identity to the VL domain sequence of antibody IMPI-011, provided that the antibody has the CDRs of antibody IMPI-011. D91. The antibody according to clause D80, wherein the variable heavy (VH) domain sequence comprises a sequence having at least 90% identity to the VH domain sequence of antibody IMPI-044 and the variable light (VL) domain sequence comprises a sequence having at least 90% identity to the VL domain sequence of antibody IMPI-044, provided that the antibody has the CDRs of antibody IMPI-044. D92. The antibody according to clause D80, wherein the variable heavy (VH) domain sequence comprises a sequence having at least 90% identity to the VH domain sequence of antibody IMPI-046 and the variable light (VL) domain sequence comprises a sequence having at least 90% identity to the VL domain sequence of antibody IMPI-046, provided that the antibody has the CDRs of antibody IMPI-046. D93. The antibody according to clause D80, wherein the variable heavy (VH) domain sequence comprises a sequence having at least 90% identity to the VH domain sequence of antibody IMPI-051 and the variable light (VL) domain sequence comprises a sequence having at least 90% identity to the VL domain sequence of antibody IMPI-051, provided that the antibody has the CDRs of antibody IMPI-051. D94. The antibody according to clause D80, wherein the variable heavy (VH) domain sequence comprises a sequence having at least 90% identity to the VH domain sequence of antibody I IMPI-024 and the variable light (VL) domain sequence comprises a sequence having at least 90% identity to the VL domain sequence of antibody IMPI-024, provided that the antibody has the CDRs of antibody IMPI-024. D95. The antibody according to clause D80, wherein the variable heavy (VH) domain sequence comprises a sequence having at least 90% identity to the VH domain sequence of antibody IMPI-058 and the variable light (VL) domain sequence comprises a sequence having at least 90% identity to the VL domain sequence of antibody IMPI-058, provided that the antibody has the CDRs of antibody IMPI-058. D96. The antibody according to clause D80, wherein the variable heavy (VH) domain sequence comprises a sequence having at least 90% identity to the VH domain sequence of antibody IMPI-043 and the variable light (VL) domain sequence comprises a sequence having at least 90% identity to the VL domain sequence of antibody IMPI-043, provided that the antibody has the CDRs of antibody IMPI-043. D97. The antibody according to clause D80, wherein the variable heavy (VH) domain sequence comprises a sequence having at least 90% identity to the VH domain sequence of antibody IMPI-045 and the variable light (VL) domain sequence comprises a sequence having at least 90% identity to the VL domain sequence of antibody IMPI-045, provided that the antibody has the CDRs of antibody IMPI-045. D98. The antibody according to clause D80, wherein the variable heavy (VH) domain sequence comprises a sequence having at least 90% identity to the VH domain sequence of antibody IMPI-027 and the variable light (VL) domain sequence comprises a sequence having at least 90% identity to the VL domain sequence of antibody IMPI-027, provided that the antibody has the CDRs of antibody IMPI-027. D99. The antibody according to clause D80, wherein the variable heavy (VH) domain sequence comprises a sequence having at least 90% identity to the VH domain sequence of antibody IMPI-018 and the variable light (VL) domain sequence comprises a sequence having at least 90% identity to the VL domain sequence of antibody IMPI-018, provided that the antibody has the CDRs of antibody IMPI-018. D100. The antibody according to clause D80, wherein the variable heavy (VH) domain sequence comprises a sequence having at least 90% identity to the VH domain sequence of antibody IMPI-048 and the variable light (VL) domain sequence comprises a sequence having at least 90% identity to the VL domain sequence of antibody IMPI-048, provided that the antibody has the CDRs of antibody IMPI-048. D101. The antibody according to clause D80, wherein the variable heavy (VH) domain sequence comprises a sequence having at least 90% identity to the VH domain sequence of antibody IMPI-033 and the variable light (VL) domain sequence comprises a sequence having at least 90% identity to the VL domain sequence of antibody IMPI-033, provided that the antibody has the CDRs of antibody IMPI-033. D102. The antibody according to clause D80, wherein the variable heavy (VH) domain sequence comprises a sequence having at least 90% identity to the VH domain sequence of antibody IMPI-014 and the variable light (VL) domain sequence comprises a sequence having at least 90% identity to the VL domain sequence of antibody IMPI-014, provided that the antibody has the CDRs of antibody IMPI-014. D103. The antibody according to clause D80, wherein the variable heavy (VH) domain sequence comprises a sequence having at least 90% identity to the VH domain sequence of antibody IMPI-038 and the variable light (VL) domain sequence comprises a sequence having at least 90% identity to the VL domain sequence of antibody IMPI-038, provided that the antibody has the CDRs of antibody IMPI-038. D104. The antibody according to clause D80, wherein the variable heavy (VH) domain sequence comprises a sequence having at least 90% identity to the VH domain sequence of antibody IMPI-068 and the variable light (VL) domain sequence comprises a sequence having at least 90% identity to the VL domain sequence of antibody IMPI-068, provided that the antibody has the CDRs of antibody IMPI-068. D105. An antibody according to any one of clauses D1 to D3, wherein the antibody comprises a variable heavy (VH) domain sequence and a variable light (VL) domain sequence and wherein the variable heavy (VH) domain and variable light (VL) domain sequences respectively comprise the variable heavy (VH) and variable light (VL) domain sequences of antibody IMPI-026, IMPI-034, IMPI-016, IMPI-050, IMPI-049, IMPI-015, IMPI-009, IMPI-011, IMPI-044, IMPI-046, IMPI-051, IMPI-024, IMPI-058, IMPI-043, IMPI-045, IMPI-027, IMPI-018, IMPI-048, IMPI-033, IMPI-014, IMPI-038 or IMPI-068.

D106. An antibody according to clause D2, wherein the antibody comprises a variable heavy (VH) domain sequence and a variable light (VL) domain sequence and wherein the variable heavy (VH) domain and variable light (VL) domain sequences respectively comprise the variable heavy (VH) and variable light (VL) domain sequences of antibody IMPI-024, IMPI-027, IMPI-038 or IMPI-068.

D107. An antibody according to clause D3, wherein the antibody comprises a variable heavy (VH) domain sequence and a variable light (VL) domain sequence and wherein the variable heavy (VH) domain and variable light (VL) domain sequences respectively comprise the variable heavy (VH) and variable light (VL) domain sequences of antibody IMPI-027, IMPI-033, IMPI-038 or IMPI-068.

D108. An anti-SARS-CoV-2 antibody, wherein the antibody comprises a variable heavy (VH) domain sequence and a variable light (VL) domain sequence and wherein the variable heavy (VH) domain and variable light (VL) domain sequences respectively comprise the variable heavy (VH) and variable light (VL) domain sequences of antibody IMPI-026, IMPI-034, IMPI-016, IMPI-050, IMPI-049, IMPI-015, IMPI-009, IMPI-011, IMPI-044, IMPI-046, IMPI-051, IMPI-024, IMPI-058, IMPI-043, IMPI-045, IMPI-027, IMPI-018, IMPI-048, IMPI-033, IMPI-014, IMPI-038 or IMPI-068.

D109. The antibody according to clause D105 or D108, wherein the variable heavy (VH) domain sequence comprises the VH domain sequence of antibody IMPI-026 and the variable light (VL) domain sequence comprises the VL domain sequence of antibody IMPI-026.

D110. The antibody according to clause D105 or D108, wherein the variable heavy (VH) domain sequence comprises the VH domain sequence of antibody IMPI-034 and the variable light (VL) domain sequence comprises the VL domain sequence of antibody IMPI-034.

D111. The antibody according to clause D105 or D108, wherein the variable heavy (VH) domain sequence comprises the VH domain sequence of antibody IMPI-016 and the variable light (VL) domain sequence comprises the VL domain sequence of antibody IMPI-016.

D112. The antibody according to clause D105 or D108, wherein the variable heavy (VH) domain sequence comprises the VH domain sequence of antibody IMPI-050 and the variable light (VL) domain sequence comprises the VL domain sequence of antibody IMPI-050.

D113. The antibody according to clause D105 or D108, wherein the variable heavy (VH) domain sequence comprises the VH domain sequence of antibody IMPI-049 and the variable light (VL) domain sequence comprises the VL domain sequence of antibody IMPI-049.

D114. The antibody according to clause D105 or D108, wherein the variable heavy (VH) domain sequence comprises the VH domain sequence of antibody IMPI-015 and the variable light (VL) domain sequence comprises the VL domain sequence of antibody IMPI-015.

D115. The antibody according to clause D105 or D108, wherein the variable heavy (VH) domain sequence comprises the VH domain sequence of antibody IMPI-009 and the variable light (VL) domain sequence comprises the VL domain sequence of antibody IMPI-009.

D116. The antibody according to clause D105 or D108, wherein the variable heavy (VH) domain sequence comprises the VH domain sequence of antibody IMPI-011 and the variable light (VL) domain sequence comprises the VL domain sequence of antibody IMPI-011.

D117. The antibody according to clause D105 or D108, wherein the variable heavy (VH) domain sequence comprises the VH domain sequence of antibody IMPI-044 and the variable light (VL) domain sequence comprises the VL domain sequence of antibody IMPI-044.

D118. The antibody according to clause D105 or D108, wherein the variable heavy (VH) domain sequence comprises the VH domain sequence of antibody IMPI-046 and the variable light (VL) domain sequence comprises the VL domain sequence of antibody IMPI-046.

D119. The antibody according to clause D105 or D108, wherein the variable heavy (VH) domain sequence comprises the VH domain sequence of antibody IMPI-051 and the variable light (VL) domain sequence comprises the VL domain sequence of antibody IMPI-051.

D120. The antibody according to clause D105 or D108, wherein the variable heavy (VH) domain sequence comprises the VH domain sequence of antibody IMPI-024 and the variable light (VL) domain sequence comprises the VL domain sequence of antibody IMPI-024.

D121. The antibody according to clause D105 or D108, wherein the variable heavy (VH) domain sequence comprises the VH domain sequence of antibody IMPI-058 and the variable light (VL) domain sequence comprises the VL domain sequence of antibody IMPI-058.

D122. The antibody according to clause D105 or D108, wherein the variable heavy (VH) domain sequence comprises the VH domain sequence of antibody IMPI-043 and the variable light (VL) domain sequence comprises the VL domain sequence of antibody IMPI-043.

D123. The antibody according to clause D105 or D108, wherein the variable heavy (VH) domain sequence comprises the VH domain sequence of antibody IMPI-045 and the variable light (VL) domain sequence comprises the VL domain sequence of antibody IMPI-045.

D124. The antibody according to clause D105 or D108, wherein the variable heavy (VH) domain sequence comprises the VH domain sequence of antibody IMPI-027 and the variable light (VL) domain sequence comprises the VL domain sequence of antibody IMPI-027.

D125. The antibody according to clause D105 or D108, wherein the variable heavy (VH) domain sequence comprises the VH domain sequence of antibody IMPI-018 and the variable light (VL) domain sequence comprises the VL domain sequence of antibody IMPI-018.

D126. The antibody according to clause D105 or D108, wherein the variable heavy (VH) domain sequence comprises the VH domain sequence of antibody IMPI-048 and the variable light (VL) domain sequence comprises the VL domain sequence of antibody IMPI-048.

D127. The antibody according to clause D105 or D108, wherein the variable heavy (VH) domain sequence comprises the VH domain sequence of antibody IMPI-033 and the variable light (VL) domain sequence comprises the VL domain sequence of antibody IMPI-033.

D128. The antibody according to clause D105 or D108, wherein the variable heavy (VH) domain sequence comprises the VH domain sequence of antibody IMPI-014 and the variable light (VL) domain sequence comprises the VL domain sequence of antibody IMPI-014.

D129. The antibody according to clause D105 or D108, wherein the variable heavy (VH) domain sequence comprises the VH domain sequence of antibody IMPI-038 and the variable light (VL) domain sequence comprises the VL domain sequence of antibody IMPI-038.

D130. The antibody according to clause D105 or D108, wherein the variable heavy (VH) domain sequence comprises the VH domain sequence of antibody IMPI-068 and the variable light (VL) domain sequence comprises the VL domain sequence of antibody IMPI-068.

D131. The antibody according to any one of clauses D1 to D3, comprising VH and/or VL domain framework regions of human germline gene segment sequences.

D132. The antibody according to any one of clauses D1 to D3 or D131, comprising an antibody VH domain which
i) is derived from recombination of a human heavy chain V gene segment, a human heavy chain D gene segment and a human heavy chain J gene segment, wherein the V gene segment is IGHV5-51*01, IGHV4-31*03 or IGHV3-30*18;
and/or the J gene segment is IGHJ4*02 or IGHJ6*02, or
ii) comprises framework regions FR1, FR2, FR3 and FR4, wherein FR1 aligns with human germline V gene segment IGHV5-51*01, IGHV4-31*03 or IGHV3-30*18 with up to 1, 2, 3, 4, or 5 amino acid alterations, FR2 aligns with human germline V gene segment IGHV5-51*01, IGHV4-31*03 or IGHV3-30*18 with up to 1, 2, 3, 4, or 5 amino acid alterations, FR3 aligns with human germline V gene segment IGHV5-51*01, IGHV4-31*03 or IGHV3-30*18 with up to 1, 2, 3, 4 or 5 amino acid alterations, and/or FR4 aligns with human germline J gene segment IGHJ4*02 or IGHJ6*02 with up to 1, 2, 3, 4 or 5 amino acid alterations.

D133. The antibody according to any one of clauses D1 to D3, D131 or D132, comprising an antibody VH domain which
i) is derived from recombination of a human heavy chain V gene segment IGHV5-51*01, IGHV4-31*03 or IGHV3-30*18, a human heavy chain D gene segment and a human heavy chain J gene segment, or
ii) comprises framework regions FR1, FR2, FR3 and FR4, wherein FR1, FR2 and FR3 each align with human germline V segment IGHV5-51*01, IGHV4-31*03 or IGHV3-30*18 with up to 1, 2, 3, 4 or 5 amino acid alterations.

D134. The antibody according to any one of clauses D1 to D3 or D131 to D133, wherein the J gene segment is IGHJ4*02 or IGHJ6*02, or wherein the VH domain framework region FR4 aligns with human germline J gene segment IGHJ4*02 or IGHJ6*02 with 1, 2, 3, 4 or 5 amino acid alterations. D135. The antibody according to any one of clauses D1 to D3 or D131, comprising an antibody VL domain which
i) is derived from recombination of a human light chain V gene segment and a human light chain J gene segment, wherein the V gene segment is IGKV1D-13*d01 or IGKV1-12*01, and/or the J gene segment is IGKJ1*01, IGKJ4*01 or IGKJ3*01; or
ii) comprises framework regions FR1, FR2, FR3 and FR4, wherein FR1 aligns with human germline V gene segment IGKV1D-13*d01 or IGKV1-12*01 with up to 1, 2, 3, 4, or 5 amino acid alterations, FR2 aligns with human germline V gene segment IGKV1D-13*d01 or IGKV1-12*01 with up to 1, 2, 3, 4, or 5 amino acid alterations FR3 aligns with human germline V gene segment IGKV1D-13*d01 or IGKV1-12*01 with up to 1, 2, 3, 4 or 5 amino acid alterations, and/or FR4 aligns with human germline J gene segment IGKJ1*01, IGKJ4*01 or IGKJ3*01 with up to 1, 2, 3, 4 or 5 amino acid alterations.

D136. The antibody according to any one of clauses D1 to D3 or D131, comprising an antibody VL domain derived from recombination of a human light chain V gene segment and a human light chain J gene segment, wherein:
the V gene segment is IGKV1D-13*d01 or IGKV1-12*01, and optionally the J gene segment is IGKJ1*01, IGKJ4*01 or IGKJ3*01.

E1. The antibody according to any one of clauses A 1 to A109, B1 to B106, C1 to C63, or D1 to D130, wherein the antibody is a human IgG1.

E2. The antibody according to clause E1, wherein the antibody is a human IgG1 comprising a constant region sequence of SEQ ID NO: 418.

E3. The antibody according to any one of clauses A 1 to A109, B1 to B106, C1 to C63, or D1 to D130, wherein the antibody is a human IgG4.

E4. The antibody according to clause E3, wherein the antibody is a human IgG4 comprising a constant region sequence of SEQ ID NO: SEQ ID NO: 436.

E5. The antibody according to any one of clauses A1 to A109, B1 to B106, C1 to C63, D1 to D130, or E1 to E6 wherein the antibody comprises kappa (κ) light chain constant regions, preferably wherein the kappa (κ) light chain constant regions sequence is SEQ ID NO: 448.

E6. A nucleic acid comprising a sequence that encodes a VH domain and/or an VL domain of an antibody as defined in any preceding clause.

E7. A nucleic acid comprising a sequence that encodes a VH domain and/or an VL domain of antibody IMPI-029, IMPI-056, IMPI-005, IMPI-012, IMPI-052, IMPI-002, IMPI-041, IMPI-036, IMPI-055, IMPI-054, IMPI-042, IMPI-021, IMPI-004, IMPI-047, IMPI-017, IMPI-059, IMPI-030, IMPI-053, IMPI-025, IMPI-040, IMPI-007, IMPI-020, IMPI-032, IMPI-023, IMPI-039, IMPI-001, IMPI-019, IMPI-010, IMPI-008, IMPI-031, IMPI-057, IMPI-022, IMPI-035, IMPI-067, IMPI-072, IMPI-003, IMPI-013, IMPI-063, IMPI-061, IMPI-062, IMPI-064, IMPI-065, IMPI-066, IMPI-069, IMPI-070, IMPI-071; IMPI-026, IMPI-034, IMPI-016, IMPI-050, IMPI-049, IMPI-015, IMPI-009, IMPI-011, IMPI-044, IMPI-046, IMPI-051, IMPI-024, IMPI-058, IMPI-043, IMPI-045, IMPI-027, IMPI-018, IMPI-048, IMPI-033, IMPI-014, IMPI-028, IMPI-038 or IMPI-068.

E8. A nucleic acid comprising a sequence that encodes the VH domain of antibody IMPI-029, IMPI-056, IMPI-005, IMPI-012, IMPI-052, IMPI-002, IMPI-041, IMPI-036, IMPI-055, IMPI-054, IMPI-042, IMPI-021, IMPI-004, IMPI-047, IMPI-017, IMPI-059, IMPI-030, IMPI-053, IMPI-025, IMPI-040, IMPI-007, IMPI-020, IMPI-032, IMPI-023, IMPI-039, IMPI-001, IMPI-019, IMPI-010, IMPI-008, IMPI-031, IMPI-057, IMPI-022, IMPI-035, IMPI-067, IMPI-072, IMPI-003, IMPI-013, IMPI-063, IMPI-061, IMPI-062, IMPI-064, IMPI-065, IMPI-066, IMPI-069, IMPI-070, IMPI-071; IMPI-026, IMPI-034, IMPI-016, IMPI-050, IMPI-049, IMPI-015, IMPI-009, IMPI-011, IMPI-044, IMPI-046, IMPI-051, IMPI-024, IMPI-058, IMPI-043, IMPI-045, IMPI-027, IMPI-018, IMPI-048, IMPI-033, IMPI-014, IMPI-028, IMPI-038 or IMPI-068.

E9. A nucleic acid comprising a sequence that encodes the VL domain of antibody IMPI-029, IMPI-056, IMPI-005, IMPI-012, IMPI-052, IMPI-002, IMPI-041, IMPI-036, IMPI-055, IMPI-054, IMPI-042, IMPI-021, IMPI-004, IMPI-047, IMPI-017, IMPI-059, IMPI-030, IMPI-053, IMPI-025, IMPI-040, IMPI-007, IMPI-020, IMPI-032, IMPI-023, IMPI-039, IMPI-001, IMPI-019, IMPI-010, IMPI-008, IMPI-031, IMPI-057, IMPI-022, IMPI-035, IMPI-067, IMPI-072, IMPI-003, IMPI-013, IMPI-063, IMPI-061, IMPI-062, IMPI-064, IMPI-065, IMPI-066, IMPI-069, IMPI-070, IMPI-071; IMPI-026, IMPI-034, IMPI-016, IMPI-050, IMPI-049, IMPI-015, IMPI-009, IMPI-011, IMPI-044, IMPI-046, IMPI-051, IMPI-024, IMPI-058, IMPI-043, IMPI-045, IMPI-027, IMPI-018, IMPI-048, IMPI-033, IMPI-014, IMPI-028, IMPI-038 or IMPI-068.

E10. A vector comprising the nucleic acid of any one of clauses E6 to E9; optionally wherein the vector is a CHO vector.

E11. A host cell comprising the nucleic acid of any one of clauses E6 to E9 or the vector of clause E10. E12. A pharmaceutical composition comprising an antibody according to any one of clauses A1 to A 115, B1 to B 112, C1 to C69, D1 to D136 or E1 to E5 and a pharmaceutically acceptable excipient. E13. A pharmaceutical composition comprising an isolated nucleic acid encoding an antibody according to any one of clauses A1 to A 115, B1 to B 112, C1 to C69, D1 to D136 or E1 to E5, or the isolated nucleic acid of any one of clauses E8 to E 11 and a pharmaceutically acceptable excipient.

E14. The pharmaceutical composition according to clause E12 or E13, formulated for intravenous, intramuscular or subcutaneous administration.

E15. The pharmaceutical composition according to any of clauses E12 to E14, further comprising at least one further therapeutic agent.

E16. The pharmaceutical composition of clause E15, wherein the further therapeutic agent is at least one, preferably one or two, further antibodies.

E17. The pharmaceutical composition of clause E16, wherein the at least one further antibody is selected from:
a. an antibody that specifically binds to the receptor binding domain (RBD) of the S1 subunit of the SARS-CoV-2 spike protein and competes for binding to the SARS-CoV-2 spike protein with the human ACE2 receptor;
b. an antibody that specifically binds to the receptor binding domain (RBD) of the S1 subunit of the SARS-CoV-2 spike protein and does not compete for binding to the SARS-CoV-2 spike protein with the human ACE2 receptor;
c. an antibody that specifically binds to the N-terminal domain (NTD) of the S1 subunit of the of SARS-CoV-2 spike protein;
d. an antibody that specifically binds to the S2 subunit of the of SARS-CoV-2 spike protein; and e. an antibody preferentially binds to the trimer form of the SARS-CoV-2 spike protein over the isolated RBD domain, S1 subunit and S2 subunit ofthe SARS-CoV-2 spike protein.

E18. A kit comprising the pharmaceutical composition of any one of clauses E12 to E17.

E19. A kit comprising the pharmaceutical composition of any one of clauses E12 to E14.

E20. The kit according to clause E19 further comprising at least one further therapeutic agent.

E21. The kit according to clause E20, wherein the further therapeutic agent is a further pharmaceutical composition comprising at least one, preferably one or two, further antibodies.

E22. The kit according to clause E21, wherein the at least one further antibody is selected from: a. an antibody that specifically binds to the receptor binding domain (RBD) of the S1 subunit of the SARS-CoV-2 spike protein and competes for binding to the SARS-CoV-2 spike protein with the human ACE2 receptor;
b. an antibody that specifically binds to the receptor binding domain (RBD) of the S1 subunit of the SARS-CoV-2 spike protein and does not compete for binding to the SARS-CoV-2 spike protein with the human ACE2 receptor;
c. an antibody that specifically binds to the N-terminal domain (NTD) of the S1 subunit of the of SARS-CoV-2 spike protein;
d. an antibody that specifically binds to the S2 subunit of the of SARS-CoV-2 spike protein; and e. an antibody preferentially binds to the trimer form of the SARS-CoV-2 spike protein over the isolated RBD domain, S1 subunit and S2 subunit ofthe SARS-CoV-2 spike protein.

E23. The kit according to any of clauses E18 and E22, further comprising a label or instructions for use to treat and/or prevent a SARS-CoV-2-related disease or condition, such as COVID-19, in a human; optionally wherein the label or instructions comprise a marketing authorisation number (e.g., an FDA or EMA authorisation number); optionally wherein the kit comprises an IV or injection device that comprises the antibody or fragment.

E24. The antibody according to any one of clauses A1 to A115, B1 to B112, C1 to C69, D1 to D136 or E1 to E5, or the composition according to any one of clauses E12 to E19, for use as a medicament. E25. The antibody according to any one of clauses A1 to A115, B1 to B112, C1 to C69, D1 to D136 or E1 to E5, or the composition according to any one of clauses E12 to E19, for use in a method of treating a SARS-CoV-2-related disease or condition, said method comprising administering the antibody or composition to a patient.

E26. The antibody according to any one of clauses A1 to A115, B1 to B112, C1 to C69, D1 to D136 or E1 to E5, or the composition according to any one of clauses E12 to E19, for use in a method of preventing a SARS-CoV-2-related disease or condition, said method comprising administering the antibody or composition to a patient.

E27. Use of an antibody according to any one of clauses A1 to A115, B1 to B112, C1 to C69, D1 to D136 or E1 to E5, or the composition according to any one of clauses E12 to E19, in the manufacture of a medicament for use in a method of treating a SARS-CoV-2-related disease or condition.

E28. Use of an antibody according to any one of clauses A1 to A115, B1 to B112, C1 to C69, D1 to D136 or E1 to E5, or the composition according to any one of clauses E12 to E19, in the manufacture of a medicament for use in a method of preventing a SARS-CoV-2-related disease or condition.

E29. A method of treating a SARS-CoV-2-related disease or condition in a human, comprising administering to said human a therapeutically effective amount of an antibody according to any one of clauses A1 to A115, B1 to B 112, C1 to C69, D1 to D136 or E1 to E5, or the composition according to any one of clauses E12 to E19.

E30. A method of preventing a SARS-CoV-2-related disease or condition in a human, comprising administering to said human a therapeutically effective amount of an antibody according to any one of clauses A1 to A 115, B1 to B112, C1 to C69, D1 to D136 or E1 to E5, or the composition according to any one of clauses E12 to E19.

E31. The antibody for use according to clause E25 or E26, or the composition for use according to clause E25 or E26, or the use of an antibody according to clause E27 or E28, or the method according to clause E29 or E30, wherein the SARS-CoV-2-related disease or condition is COVID-19.

E32. The antibody for use according to any one of clauses E25, E26 or E31, or the composition for use according to any one of clauses E25, E26 or E31, or the use of an antibody according to any one of clauses E27, E28 or E31, or the method according to any one of clauses E29, E30 or E32, said method further comprising administering at least one further therapeutic agent.

E33. The antibody for use according to any one of clauses E25, E26, E31 or E32, or the composition for use according to any one of clauses E25, E26, E31 or E32, or the use of an antibody according to any one of clauses E27, E28, E31 or E32, or the method according to any one of clauses E29, E30, E31 or E32, wherein administration of the further therapeutic agent is simultaneous, separate or sequential.

E34. The antibody for use according to any one of clauses E25, E26, or E31 to E33, or the composition for use according to any one of clauses E25, E26, or E31 to E33, or the use of an antibody according to any one of clauses E27, E28, or E31 to E33, or the method according to any one of clauses E29, E30, or E31 to E33, wherein the further therapeutic agent is at least one, preferably one or two, further antibodies.

E35. The antibody for use according to any one of clauses E25, E26, or E31 to E34, or the composition for use according to any one of clauses E25, E26, or E31 to E34, or the use of an antibody according to any one of clauses E27, E28, or E31 to E34, or the method according to any one of clauses E29, E30, or E31 to E34, wherein the at least one further antibody is selected from:
a. an antibody that specifically binds to the receptor binding domain (RBD) of the S1 subunit of the SARS-CoV-2 spike protein and competes for binding to the SARS-CoV-2 spike protein with the human ACE2 receptor;
b. an antibody that specifically binds to the receptor binding domain (RBD) of the S1 subunit of the SARS-CoV-2 spike protein and does not compete for binding to the SARS-CoV-2 spike protein with the human ACE2 receptor;
c. an antibody that specifically binds to the N-terminal domain (NTD) of the S1 subunit of the of SARS-CoV-2 spike protein;
d. an antibody that specifically binds to the S2 subunit of the of SARS-CoV-2 spike protein; and e. an antibody preferentially binds to the trimer form of the SARS-CoV-2 spike protein over the isolated RBD domain, S1 subunit and S2 subunit ofthe SARS-CoV-2 spike protein.

E36. Use of an antibody according to any of clauses A1 to A115, B1 to B112, C1 to C69, D1 to D136 or E1 to E5, for determining the presence or absence of SARS-CoV-2 in a sample.

E37. A method of determining the presence or absence of SARS-CoV-2, in a sample, the method comprising contacting the sample with an antibody according to any of clauses A1 to A115, B1 to B112, C1 to C69, D1 to D136 or E1 to E5; and testing for binding between the antibody and SARS-CoV-2 in the sample;
wherein detection of binding indicates the presence of SARS-CoV-2 in the sample and wherein absence of binding indicates the absence of SARS-CoV-2 in the sample.

E38. Use according to clause E36 or a method according to clause E37, wherein the antibody comprises or is conjugated to a detectable label.

E39. Use according to clause E36 or clause E38, or a method according to clause E37 or clause E38, wherein the sample has been obtained from a human who has been or is suspected of having been infected with SARS-CoV-2 and/or who exhibits one or more symptoms of a SARS-CoV-2-related disease or condition, such as COVID-19.

E40. Use or a method according to any of clauses E36, E38 or E39, or a method according to any one of clauses E37 to E40, wherein the sample is a serum, plasma, or whole blood sample, or an oral or nasal swab, urine, faeces, or cerebrospinal fluid (CFS), or wherein the sample is from any suspected SARS-CoV-2 infected organ or tissue.

E41. A diagnostic kit for the use as set out in any of clauses E36 or E38 to E40, or the method as set out in any of clauses E37 to E40, comprising an antibody according to any of clauses A1 to A 115, B1 to B 112, C1 to C69, D1 to D136 or E1 to E5, and optionally one or more buffering solutions. E42. A diagnostic kit according to clause E41, wherein the antibody comprises or is conjugated to a detectable label.

E43. A diagnostic kit according to clause E42, comprising a first reagent comprising the antibody according to any of clauses A1 to A 115, B1 to B 112, C1 to C69, D1 to D136 or E1 to E5, and a second reagent comprising a detector molecule that binds to the first reagent.

E44. A diagnostic kit according to clause E43, wherein the detector molecule is an antibody that comprises or is conjugated to a detectable label.

### EXAMPLES

Here we describe antibodies binding to the spike protein of SARS-CoV-2 which are suitable for use in treating COVID19 disease. Through immunising transgenic mice which generate fully human antibodies and testing a wide diversity of antibodies in a series of biologically relevant assays, we were able to obtain spike-binding monoclonal antibodies which neutralise the entry of SARS-CoV-2 into target cells. These antibodies target multiple domains on the spike protein, including S1 (e.g., RBD) and/or S2, and can be used alone or in combination as a therapeutic or prophylactic against SARS-CoV-2 (e.g., for administration to human patients) or as a diagnostic for detecting SARS-CoV-2, (e.g., in in vitro diagnostic test assays and kits).

### Example 1. Generation of Sars-Cov-2 Spike Antigens and Anti-Spike Antibodies

Kymab Darwin transgenic mice, which produce human antibodies, were immunised with SARS-CoV-2 spike in a variety of immunisation regimens and immunogenic formats, and antigen-specific B cells were selected from the immunised mice. Single B cells from spleen, lymph node and bone marrow samples were sorted using a combination of plasma cell sorting and antigen specific probes. Immunogenic formats included (i) nucleic acid encoding full length spike protein (sequence as depicted in FIG. 2B) and/or a (ii) soluble trimeric spike extracellular domain protein (ECD) comprising a C-terminal T4 fibritin (foldon) trimerisation motif (Meier et al., J Mol Biol. 2004 Dec. 3; 344(4):1051-69). This soluble version ends at glutamine Q1208 of the pre-fusion sequence followed by a GGGGSGGGGS linker, the T4 fibritin (foldon) trimerization motif, a further GGGGSGGGGS linker, the Myc tag, a GSGSGS linker and finally an 8xHIS tag to enable purification of the soluble recombinant protein. Sorting probes used were: soluble trimeric spike extracellular domain protein comprising C-terminal T4 fibritin (foldon) trimerisation motif, soluble spike receptor binding domain (RBD) protein (monomeric) and full length trimeric spike protein presented on green fluorescent protein (GFP) virus like particles (VLPs) generated from host cells transfected with DNA encoding full length spike protein. All constructs that encoded the spike protein contained the double proline stabilising mutations depicted in FIG. 2B.

In a first study, 8,219 B-cells were recovered after B cell sorting from immunised mice. From 224 of these B-cells, complete antibody heavy and light chain encoding nucleic acids were recovered, and expressed as fully human IgG1 antibodies in mammalian host cells to be taken forward for screening.

In a second study, 2,183 B cells were recovered after B cell sorting from immunised mice, and from 268 of these B-cells, complete antibody heavy and light chain encoding nucleic acids were recovered, and expressed as fully human IgG1 antibodies in mammalian host cells to be taken forward for screening

All antibodies taken forward for screening were expressed as fully human IgG1 kappa.

### Antigen/Immunogen Preparation

To generate purified proteins for use in mouse immunisations and sorting of B cells, DNA sequences were generated encoding ECD of trimeric spike protein containing stabilising double proline (PP) mutations (K968 and V969). Coding sequences were fused with a C-terminal His tag and N-terminal leader sequence, codon optimised for mammalian expression and expressed in CHO cells. ECD for immunisations also included a trimerisation motif as noted above. Protein was sequentially purified by a HisTrap HP column and a HiPrep 16/60 Sephacryl S-300 HR size exclusion chromatography (SEC) column (both from GE Healthcare). Purified protein was first analyzed by Native-PAGE and Western blot, and then filtered through a 0.22 μm membrane, aliquoted and stored at −80° C.

Stable cell lines expressing the SARS-CoV-2 full-length trimeric spike protein were created to generate virus like particles for sorting B cells. A full length DNA sequence encoding trimeric spike protein containing a double proline stabilising mutation (K968 and V969) was cloned into an expression vector under the control of the CMV promoter flanked by 3′ and 5′ piggyBac specific terminal repeat sequences, which facilitated stable integration into the cell genome. The expression vector contained a puromycin selection cassette to facilitate stable cell line generation. Constructs were transfected into HEK293 cell line, cultured under puromycin selection for 2 weeks and spike cell surface expression was validated by using flow cytometry to check for antigen surface expression using anti-spike monoclonal antibodies (cross-reactive anti-RBD SARS-CoV-1 antibody CR3022 obtained from Neil King, University of Washington, and anti-RBD SARS-CoV-2 antibody 40150-D001 from Sino Biologicals). VLPs were generated from HEK293 cells stably co-expressing PP stabilised trimeric spike with retroviral Group Antigens (gag) proteins and used for sorting specific B cells.

### Example 2. Antibody Binding by HTRF

A homogeneous time resolved FRET (HTRF) assay was used for primary screening to establish binding of the recovered antibodies to purified spike proteins. All antibodies were screened in an initial single point assay at 1-2 μg/mL for binding to the RBD domain (Acro Biosystems SPD-C52H3), the S1 subunit (Sino Biologicals 40591-V08H), the S2 subunit (Sino Biologicals 40590-V08B) and the stabilised soluble spike trimer protein ECD (including engineered trimerisation motif) of SARS-CoV-2 virus (see domains in FIG. 2B for reference). A summary of the primary screening results is shown in Table E2-1.

The 492 selected antibodies from Example 1 were screened in 4 batches, and 263 passed this initial HTRF screening. Criteria for passing the HTRF primary screen were that the antibody bound soluble spike trimer, S2, S1 or RBD with a ΔF value set out in Table E2-1.

TABLE E2-1
Screening from batch 1, 2, 3 and 4 Summary of number of clones meeting primary screening
selection criteria for binding to RBD, S1, S2 and spike trimer proteins. Table details
number of antibodies screened, number of positive hits and associated cut-off values.

| | Screening batch 1: | | | Screening batch 4: | | | | |
|---|---|---|---|---|---|---|---|---|
| | 151 clones | | | 83 clones | | | | |
| | Selection | No. | Selection | No. | Selection | No. | Selection | No. |
| Assay | criteria | hits | criteria | hits | criteria | hits | criteria | hits |
| Trimer | Delta F > 180 | 58 | Delta F > 50 | 71 | Delta F > 60 | 62 | Delta F > 50 | 64 |
| S2 | Delta F > 45 | 30 | Delta F > 30 | 33 | Delta F > 70 | 25 | Delta F > 40 | 39 |
| S1 | Delta F > 72 | 23 | Delta F > 10 | 23 | Delta F > 15 | 4 | Delta F > 10 | 10 |
| RBD | Delta F > 20 | 20 | Delta F > 40 | 19 | Delta F > 30 | 2 | Delta F > 10 | 6 |

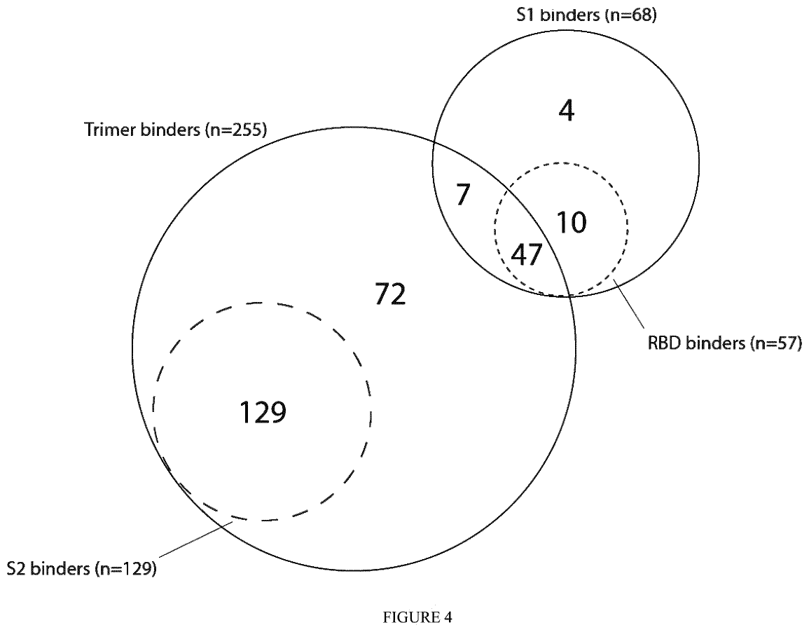

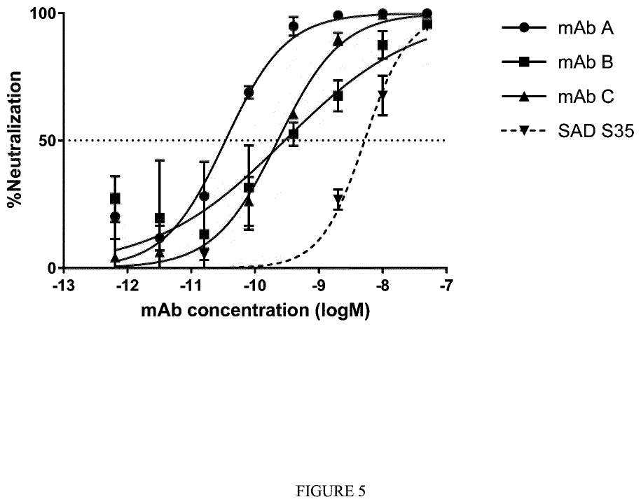

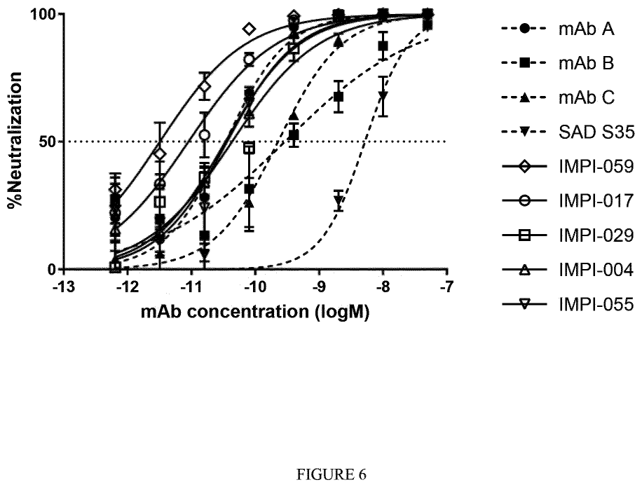

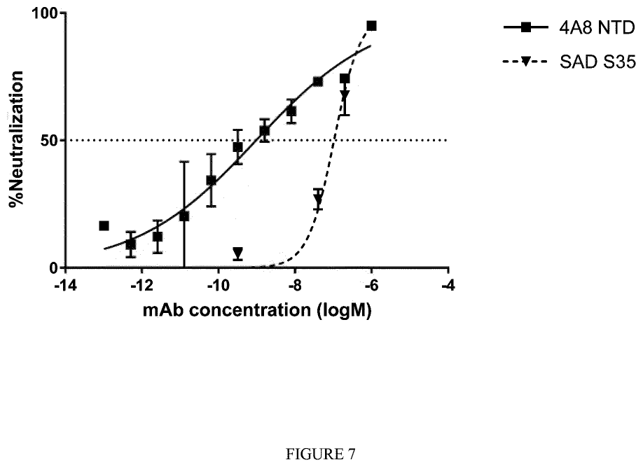

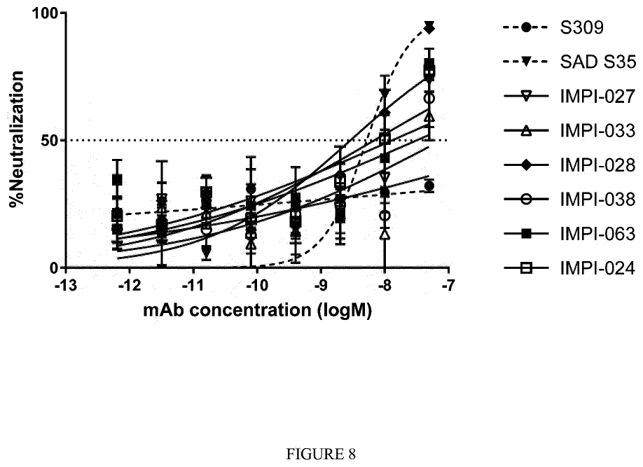

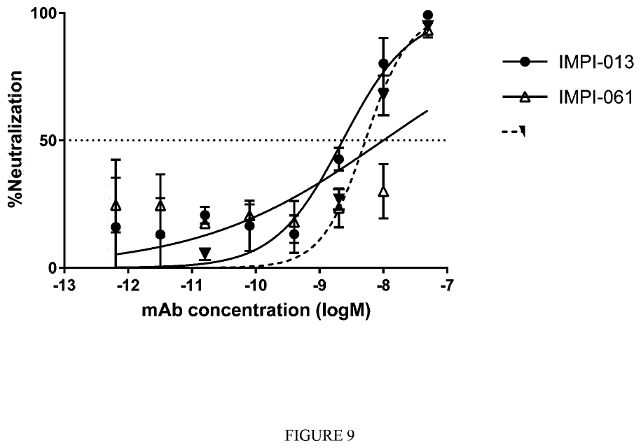

On the basis of these data, we selected clones which met the HTRF binding criteria for binding spike trimer and optionally also met the HTRF binding criteria for S1, S2 or RBD. 255 such clones were selected. No antibodies were detected that were positive (passed the criteria) for both S1 and S2 in the HTRF assay. FIG. 4 summarises numbers of antibodies binding to trimer, S1, RBD and/or S2.

Binding data (presented as deltaF) for a selection of these antibody clones are shown in Table E2-2.

TABLE E2-2
HTRF binding to SARS-COV-2 spike protein subunits.
HTRF deltaF values for binding of antibody
clones to the RBD domain, the S1 subunit,
the S2 subunit and the full-length stabilised spike
trimer protein of SARS-COV-2 virus.

| | Binding | Binding | Binding | Binding |
|---|---|---|---|---|
| Clone | trimer | RBD | S1 | S2 |
| IMPI-004 | 952.1 | 1169.9 | 1749.3 | 19.2 |
| IMPI-029 | 1704.1 | 1175.7 | 3053.1 | 4.1 |
| IMPI-056 | 1627.1 | 1262.7 | 2996.8 | 2.1 |
| IMPI-047 | 1414.2 | 899.2 | 2254.6 | 4.3 |
| IMPI-005 | 1361.0 | 608.5 | 1507.4 | −1.8 |
| IMPI-006 | 1061.4 | 330.6 | 885.1 | 40.7 |
| IMPI-055 | 550.8 | 983.3 | 1031.2 | −10.3 |
| IMPI-054 | 563.4 | 818.2 | 772.5 | 15.8 |
| IMPI-017 | 549.6 | 894.4 | 752.5 | 13.0 |
| IMPI-059 | 403.0 | 882.3 | 568.8 | −2.7 |
| IMPI-037 | 556.6 | 468.1 | 625.9 | −2.0 |
| IMPI-060 | 488.3 | 217.1 | 377.6 | 6.0 |
| IMPI-013 | 1420.4 | −10.9 | −2.8 | 684.2 |
| IMPI-028 | 480.1 | 108.0 | 253.7 | 1.3 |
| IMPI-027 | 800.4 | 318.1 | 755.2 | 7.5 |
| IMPI-033 | 884.5 | 454.2 | 929.3 | −3.6 |
| IMPI-021 | 961.8 | 596.6 | 1279.2 | −0.3 |
| IMPI-032 | 217.7 | 4.0 | −8.9 | −8.0 |
| IMPI-038 | 1307.9 | 544.4 | 1443.8 | −4.3 |
| IMPI-022 | 483.0 | −2.4 | −5.8 | −9.7 |
| IMPI-024 | 1461.1 | 225.3 | 799.4 | −15.4 |
| IMPI-002 | 912.7 | 172.1 | 541.0 | −5.8 |
| IMPI-052 | 1134.4 | 228.8 | 732.2 | −15.9 |
| IMPI-042 | 925.8 | 121.4 | 616.7 | 1.6 |
| IMPI-063 | 968.5 | −7.3 | −0.4 | 410.6 |
| IMPI-067 | 962.8 | −5.4 | 3.1 | 4.6 |
| IMPI-068 | 1595.6 | 42.3 | 612.9 | 1.4 |

Materials and methods for HTRF binding assays

Supernatants collected from suspension CHO cells transfected with expression vectors encoding human IgG1 heavy and light chains were screened for binding to the RBD domain (Acro Biosystems SPD-C52H3), the S1 subunit (Sino Biological 40591-V08H), the S2 subunit (Sino Biological 40590-V08B) and the full-length stabilised spike trimer protein (in-house) of SARS-CoV-2 virus. All proteins included a His tag. 5 μL/well of supernatants normalised between 1 and 2 μg/mL were plated into 384 well white HTRF plates (Greiner 784904-012). 5 μL/well of positive control (Sino Biological anti-SARS-COV-2 RBD antibody 40150-D001 for RBD, S1 and trimer binding assays; Sino Biological anti-SARS-COV-2 S2 antibody 40590-D001 for S2 binding assay) and negative control (non-binding human IgG1, in-house) antibodies were added to the required wells in expression medium (Gibco A1383502). Positive and negative control antibodies were added respectively at 1.2 nM, 1.2 nM, 2 nM and 4 nM for the RBD, the S1, the S2 and the trimer binding assays to give final concentrations of 0.3 nM, 0.3 nM, 0.5 nM and lnM. 5 μL/well of RBD diluted at 4 nM, S1 at 40 nM, S2 at 20 nM or spike trimer at 20 nM in HTRF buffer (PBS (Gibco 14190250, 0.1% BSA (Sigma A7906), 0.53M KF (Sigma 60238-100G-F)) were added on top on the supernatants to give final concentrations of 1 nM, 10 nM, 5 nM and 5 nM. After a 30-min incubation at room temperature, 10 μL/well of an anti-Histidine d2 monoclonal antibody (Cisbio 61HISDLB) diluted at 13.3 nM in HTRF buffer together with an anti-human IgG Europium cryptate polyclonal antibody (Cisbio 61HFCKLB) diluted respectively at 0.42 nM, 1.67 nM, 0.83 nM and 1.67 nM for the RBD, the S1, the S2 and the spike trimer binding assays were added to the wells. Plates were left to incubate at room temperature for 2 hours for the RBD, the S1 and the S2 binding assays and for 4 hours for the spike trimer binding assay. Plates were then read on the Envision (Perkin Elmer) using 320 nm for excitation and detecting fluorescence at 665 and 620 nm.

TABLE E2-3
Reagent concentrations for HTRF assay

| | Final | Final | Final |
|---|---|---|---|
| | concentration | concentration | concentration |
| | of Spike | of anti-His | of anti-human |
| HTRF Binding Assays | protein | d2 antibody | IgG antibody |
| Binding to RBD | [RBD] = 1 nM | 6.67 nM | 0.21 nM |
| Binding to S1 | [S1] = 10 nM | 6.67 nM | 0.83 nM |
| Binding to S2 | [S2] = 5 nM | 6.67 nM | 0.42 nM |
| Binding to spike trimer | [trimer] = 5 nM | 6.67 nM | 0.83 nM |

The selection of hits was based on a histogram generated using Genedata Biologics (Genedata) representing the overall distribution of the Delta F values obtained for all the wells in individual binding assays. The selection criteria for each binding assay (“cut-off”) (Delta F, summarised in Table E2-1) is visually applied at the tail of the histogram when the frequency of hits decreases. Any clone positive in at least one of the HTRF assays was carried forward to a secondary screening stage.

$$Calculation of HTRF ratio for primary screening : \text{} \\ HTRF Ratio = \frac{Fluorescence at 665 nm}{Fluorescence at 620 nm} \times 10000 \qquad Equation 1$$

$$Calulation of Delta F for HTRF binding assays : Delta F = \frac{( HTRF {Ratio}_{sample} - HTRF {Ratio}_{minimum} )}{( HTRF {Ratio}_{minimum} )} \times 100 \qquad Equation 2$$

Minimum signal (HTRF Ratioₘᵢₘₘᵤₘ) is obtained from wells containing spike protein and the negative control antibody.

### Example 3. HTRF RBD:ACE2 Neutralisation Assays

The 492 selected antibodies from Example 1 were screened for their capacity to neutralise the binding between the RBD domain of the spike protein of SARS-CoV-2 virus and its ligand the human ACE2 protein. ACE2 is the cell surface protein targeted by the virus for binding and entry to the cells, thus serving as a receptor for the spike protein. All antibodies were initially screened as supernatants at a single concentration (summary shown in Table E3-1). Four batches of screening were performed, with hits (antibodies that passed the selection criteria) as follows:

TABLE E3-1
Summary of number of primary hits fulfilling the selection cut-
off resulting from HTRF RBD:ACE2 neutralisation screening

| | 151 clones | | | 83 clones | | | | |
|---|---|---|---|---|---|---|---|---|
| | Selection | No. | Selection | No. | Selection | No. | Selection | No. |
| Assay | criteria | hits | criteria | hits | criteria | hits | criteria | hits |
| Neutralisation | % Effect < 89 | 21 | % Effect < 100 | 16 | % Effect < 111 | 8 | % Effect < 105 | 5 |
| RBD/ACE2 | | | | | | | | |

Supernatants from 263 antibody-expressing CHO cells passed initial primary screening in the HTRF binding assay of Example 2 and/or in the above neutralisation assay of Example 3. These 263 antibodies were purified and re-screened again as full titrations to generate IC50 values in the RBD:ACE2 neutralisation assay. Results and IC50 values (where it was possible to calculate) for a selection of these antibodies are shown in Table E3-2.

TABLE E3-2
HTRF neutralisation IC50 values

| | Neutralisation | Neutralisation Spike |
|---|---|---|
| Clone | RBD:ACE2 (IC50, M) | trimer:ACE2 (IC50, M) |
| IMPI-004 | 2.42E−09 | 4.09E−09 |
| IMPI-029 | 1.26E−09 | 3.31E−09 |
| IMPI-056 | 1.93E−09 | 3.73E−09 |
| IMPI-047 | 3.36E−09 | 7.30E−09 |
| IMPI-005 | 3.18E−09 | 1.31E−08 |
| IMPI-006 | Below threshold | inactive |
| IMPI-055 | 3.39E−09 | 4.50E−09 |
| IMPI-054 | 2.28E−09 | 1.16E−08 |
| IMPI-017 | 8.32E−10 | 3.15E−09 |
| IMPI-059 | 2.02E−09 | 8.38E−09 |
| IMPI-037 | 7.76E−09 | 1.37E−08 |
| IMPI-060 | Below threshold | 5.26E−09 |
| IMPI-013 | | Below threshold |
| IMPI-028 | | inactive |
| IMPI-027 | | Increased binding |
| IMPI-033 | | Increased binding |
| IMPI-021 | 4.87E−09 | Yes |
| IMPI-032 | | Yes |
| IMPI-038 | | Increased binding |
| IMPI-022 | | inactive |
| IMPI-024 | inactive | Increased binding |
| IMPI-002 | 8.15E−09 | Yes |
| IMPI-052 | 4.85E−09 | Yes |
| IMPI-042 | 5.54E−09 | Yes |
| IMPI-063 | | inactive |
| IMPI-067 | | Below threshold |
| IMPI-068 | | Increased binding |

“Below threshold” indicates that there was some neutralising activity but not enough to generate an IC50 value.
“Yes” indicates the antibody was tested at single concentration and there was neutralising activity, but a full titration was not performed so that IC50 values could be calculated.
“Increased binding” indicates that an increase in binding between ACE2:trimer (rather than blocking or no activity) was observed.
“Inactive” indicates that no significant neutralisation activity was observed in this specific assay.
Blank indicates that the antibody was not tested in the assay.
IMPI-013 and IMPI-063 (S2 binders) and IMPI-032, IMPI-022 and IMPI-067 (trimer binders) were not tested in the RBD:ACE2 neutralisation assay as they do not bind to RBD.

The assay using the isolated RBD domain categorised the antibodies as either neutralising or non-neutralising for the interaction between RBD and ACE2. The IC50 values of antibodies which were determined to be neutralising antibodies are shown in Table E3-2, that is a numeric IC50 value in Table E3-2 is a neutralising antibody. Antibodies which were determined to be non-neutralising for RBD in this assay are also indicated (“inactive”). Note that antibodies that do not function by inhibition of RBD and ACE2 binding will be ‘inactive’ in this assay, however, it does not mean the antibodies will be inactive in other assays.

To check for neutralising activity in antibodies that do not bind ACE2, a subset of antibodies were also tested in an HTRF assay that measures neutralisation between the full soluble trimer protein and ACE2. The assay using the spike trimer protein categorised the antibodies as (i) neutralising the spike:ACE2 interaction, (ii) not neutralising the spike:ACE2 interaction or (iii) potentiating the spike:ACE2 interaction (Table E3-2).

Neutralising antibodies were IMPI-004, IMPI-029, IMPI-056, IMPI-47, IMPI-005, IMPI-006, IMPI-055, IMPI-054, IMPI-017, IMPI-059, IMPI-037, IMPI-060, IMPI-013, IMPI-021, IMPI-032, IMPI-002, IMPI-052, IMPI-042, IMPI-067.

Non-neutralising antibodies were IMPI-028, IMPI-022, IMPI-063.

Agonist-type antibodies were IMPI-027, IMPI-033, IMPI-038, IMPI-024, IMPI-068.

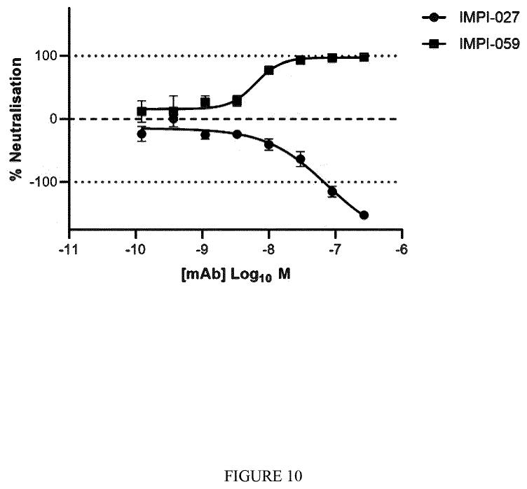

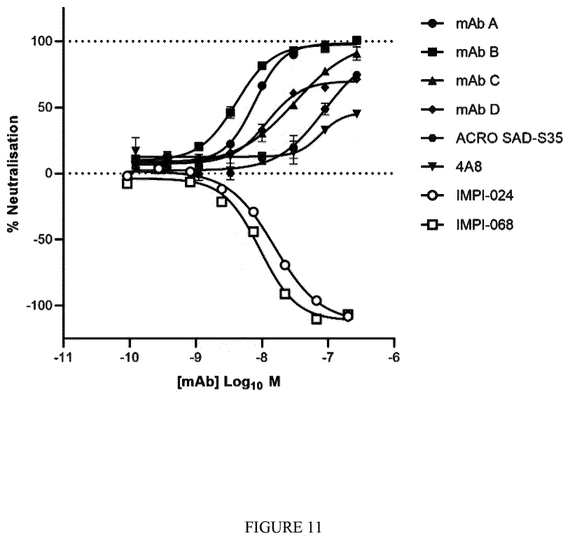

With reference to FIG. 10, IMPI-059 is an ACE2 competing neutralising antibody in this assay and shows a typical sigmoid titration curve, neutralising binding of the spike protein to ACE2 in a dose-dependent manner. IMPI-027 exhibits an unusual agonist-like activity profile, with increased binding of spike to ACE2 being observed in the presence of the antibody, in a dose-dependent manner. The same agonist-like phenomenon was seen for IMPI-024 and IMPI-068. FIG. 11. IMPI-033 and IMPI-038 behaved similarly.

As discussed elsewhere herein, a non-neutralising antibody or an agonist-type antibody may be useful therapeutically as combinations if the antibodies induce a destabilised trimeric spike protein, if they neutralise virus infection in pseudotype assays and/or live virus assays by an unusual mechanism or if they synergise with antibodies that neutralise RBD binding to ACE2.

Non-neutralising antibodies and neutralising antibodies are expected to bind to different regions of the RBD, and thus are useful in combination in DABA assays as described elsewhere herein.

Antibodies which potentiate binding between ACE2 and spike trimer protein may be especially valuable as capture antibodies for use in combination with anti-RBD antibodies which bind to different regions of the spike protein (e.g., in combination with anti-RBD antibodies which do not neutralise the ACE2:spike interaction). The potentiating antibodies may enhance binding of anti-RBD antibodies to the spike protein, thus enhance the sensitivity of detection of spike protein in a diagnostic assay.

Materials & Methods: Primary screening of antibody supernatants for neutralisation of RBD:ACE2

Supernatants collected from suspension CHO transfected with expression vectors encoding human IgG1 heavy and light chains as above were screened for neutralising the binding between the RBD domain (Acro Biosystems SPD-C52H3) of the spike protein of SARS-CoV-2 virus and its ligand the human ACE2 protein, used here directly labelled with Alexa Fluor 647. 5 μL/well of supernatants normalised between 10 and 20 μg/mL were plated into 384 well white HTRF plates (Greiner 784904-012). 5 μL/well of positive (Sino Biologicals 40150-D001) and negative control (human IgG1, in-house) antibodies were added to the required wells in expression medium (Gibco A1383502). Positive and negative control antibodies were added at 40 nM to give a final concentration of 10 nM. 5 μL/well of RBD protein diluted at 40 nM in HTRF buffer (PBS (Gibco 14190250, 0.1% BSA (Sigma A7906), 0.53M KF (Sigma 60238-100G-F)) were added on top on the supernatants to give a final concentration of 10 nM. After a 30-min incubation at room temperature, 5 μL/well of AF647-labelled human ACE2 diluted at 20 nM in HTRF buffer to give a final concentration of 5 nM was added to the wells followed 30-min later, by the addition of 5 μL/well of an anti-Histidine Europium cryptate monoclonal antibody (Cisbio 61HISKLB) diluted at 5.3 nM in HTRF buffer to the wells. Plates were left to incubate at room temperature for 2 hours and then read on an Envision plate reader (Perkin Elmer) using 320 nm for excitation and detecting fluorescence at 665 and 620 nm.

The selection of hits was based on a histogram generated using Genedata Biologics (Genedata) representing the overall distribution of the % Effect values obtained for all the wells in the neutralisation assay. The selection criteria (% Effect, summarised in Table E3-1) is visually applied at the tail of the histogram when the frequency of hits decreases. Any clone positive in the RBD:ACE2 neutralisation primary HTRF screen was carried forward to a secondary screening stage.

$$Calculation of \% of Effect for HTRF neutralisation assay : \\ \% Effect = \frac{( HTRF {Ratio}_{sample} - HTRF {Ratio}_{minimum} )}{( HTRF {Ratio}_{maximum} - HTRF {Ratio}_{minimum} )} \times 100 \qquad Equation 3$$

Wells containing 5 nM of AF647 ACE2, 10 nM of spike RBD and 10 nM of human IgG1 isotype control are referred to as maximum signal (HTRF Ratioₘₐₓᵢₘᵤₘ) and wells containing 5 nM of AF647 ACE2, 10 nM of spike RBD and 10 nM of positive control antibody (Sino Biological 40590-D001) as minimum signal (HTRF Ratioₘᵢₙᵢₘᵤₘ).

### Materials & Methods: Secondary Screening of Purified Clones for Neutralisation of RBD:ACE2

Purified antibodies identified during primary screening were screened again for neutralisation of the binding between the RBD domain (Acro Biosystems SPD-C52H3) of the spike protein of SARS-CoV-2 virus and its ligand the human ACE2 protein, used here directly labelled with Alexa Fluor 647 (in house). Purified antibodies as well as positive (Sino Biologicals 40150-D001) and negative control (human IgG1, in-house) antibodies were titrated in elution buffer (30 mM Formate, 75 mM Tris pH 7) 3-fold, 8-point dilution in duplicates starting from 360 nM to reach a final range of concentrations from 90 nM to 41 pM. Positive and negative control antibodies were also diluted at 40 nM to give a final concentration of 10 nM. 5 μL/well of titrated antibodies were plated into 384 well white HTRF plates (Greiner 784904-012). 5 μL/well of RBD protein diluted at 40 nM in HTRF buffer (PBS (Gibco 14190250, 0.1% BSA (Sigma A7906), 0.53 M KF (Sigma 60238-100G-F)) were added to the wells to give a final concentration of 10 nM. After a 30-min incubation at room temperature, 5 μL/well of AF647-labelled human ACE2 diluted at 20 nM in HTRF buffer to give a final concentration of 5 nM was added to the wells followed 30-min later, by the addition of 5 μL/well of an anti-Histidine Europium cryptate monoclonal antibody (Cisbio 61HISKLB) diluted at 5.3 nM in HTRF buffer to the wells. Plates were left to incubate at room temperature for 2 hours and then read on an Envision plate reader (Perkin Elmer) using 320 nm for excitation and detecting fluorescence at 665 and 620 nm.

$$Calculation of \% of neutralisation for HTRF neutralisation assay : \\ \% Neutralisation = 100 - \frac{( HTRF {Ratio}_{sample} - HTRF {Ratio}_{minimum} )}{( HTRF {Ratio}_{maximum} - HTRF {Ratio}_{minimum} )} \qquad Equation 4$$

Wells containing 5 nM of AF647 ACE2, 10 nM of spike RBD and 10 nM of human IgG1 isotype control are referred to as maximum signal (HTRF Ratioₘₐₓᵢₘᵤₘ) and wells containing 5 nM of AF647 ACE2, 10 nM of spike RBD and 10 nM of positive control antibody (Sino Biological 40590-D001) as minimum signal (HTRF Ratioₘᵢₙᵢₘᵤₘ).

IC50 values for purified antibodies (summarised in Table E3-2) were determined using Prism 8 (GraphPad) whereby the calculated % neutralisation for each antibody dose response was analysed using the non-linear regression, dose response, log (inhibitor) vs. response—variable slope (four parameters) in-built analysis.

### Materials & Methods: Secondary Screening of Purified Clones for Neutralisation of Spike Trimer:ACE2

Purified antibodies identified during primary screening were screened again for neutralisation of the binding between the full soluble spike trimer of SARS-CoV-2 virus and its ligand the human ACE2 protein, used here directly labelled with Alexa Fluor 647. Purified antibodies were titrated in elution buffer (30 mM Formate, 75 mM Tris pH 7) 10-fold, 2-point dilution in duplicates starting from 800 nM to reach a final range of concentrations of 200 nM and 20 nM. Positive (Sino Biologicals 40150-D001) and negative control (human IgG1, in-house) antibodies were also titrated in elution buffer 5-fold, 8-point starting from 2400 nM to reach a final range of concentrations of 600 nM to 7.68 pM. 5 μL/well of titrated antibodies were plated into 384 well white HTRF plates (Greiner 784904-012). 5 μL/well of spike trimer diluted to 20 nM in HTRF buffer (PBS (Gibco 14190250, 0.1% BSA (Sigma A7906), 0.53 M KF (Sigma 60238-100G-F)) was added to the wells to give a final concentration of 5 nM. After a 30-min incubation at room temperature, 5 μL/well of AF647-labelled human ACE2 diluted to 160 nM in HTRF buffer to give a final concentration of 40 nM was added to the wells followed 30-min later, by the addition of 5 μL/well of an anti-Histidine Europium cryptate monoclonal antibody (Cisbio 61HISKLB) diluted to 21.3 nM in HTRF buffer to the wells. Plates were left to incubate at room temperature for 4 hours and then read on an Envision plate reader (Perkin Elmer) using 320 nm for excitation and detecting fluorescence at 665 and 620 nm.

$$Calculation of \% of Effect and \% of neutralisation for neutralisation assay : \\ \% Effect = \frac{( HTRF {Ratio}_{sample} - HTRF {Ratio}_{minimum} )}{( HTRF {Ratio}_{maximum} - HTRF {Ratio}_{minimum} )} \times 1 \; 00 = 100 - \% Neutralisation \qquad Equation 3$$

Wells containing 40 nM of AF647 ACE2 and 5 nM of spike trimer are referred to as maximum signal (HTRF Ratioₘᵢₙᵢₘᵤₘ) and wells containing 40 nM of AF647 ACE2 alone as minimum signal (HTRF Ratioₘᵢₙᵢₘᵤₘ).

Antibodies identified to (i) neutralise the spike:ACE2 interaction and (iii) potentiate the spike:ACE2 interaction (Table E3-2) in the above assay were confirmed again in the spike trimer:ACE2 HTRF neutralisation assay, by performing titrations of purified antibodies in elution buffer (30 mM Formate, 75 mM Tris pH 7) 3-fold, 8-point dilution in duplicates starting from 800 nM to reach a final range of concentrations from 200 nM to 91.4 pM. IC50 values for purified antibodies (summarised in Table E3-2) were calculated using Prism 8 (GraphPad) whereby the calculated % neutralisation for each antibody dose response was analysed using the non-linear regression, dose response, log (inhibitor) vs. response—variable slope (four parameters) in-built analysis.

### Example 4. Pseudovirus Neutralisation Assay

The 255 trimer-binding antibodies which met the HTRF ΔF criteria described in Example 2, 4 further anti-S1 antibodies which met the HTRF ΔF criteria for S1 binding (but not for trimer binding) from Example 2, and 4 additional neutralising antibodies which were positive in the neutralising HTRF assay (see Example 3), provided a total of 263 candidate antibodies. We screened these further to evaluate their activity in a pseudovirus neutralisation assay using non-replication-competent pseudoviral particles with wild type SARS-CoV-2 spike trimer protein in the pseudoviral envelope.

Purified antibodies identified during primary HTRF screening as binding to SARS-Cov-2 spike trimer, were tested for neutralisation of the SARS-CoV-2 spike pseudovirus in a high throughput, 384-well format, cell-based viral neutralization assay developed at Kymab (adapted from Ferrera and Temperton, Methods Protoc. 2018 March; 1(1): 8). All trimer binders were initially tested as two-point titrations (100 nM and 10 nM) and all antibodies showing activity in this assay were run as full 8 point titrations, starting from 50 nM. Antibody titrations were incubated with SARS-CoV-2 pseudovirus particles encoding firefly luciferase before adding to Lenti-X 293 cells transiently transfected with DNA plasmids encoding human recombinant ACE2 and TMPRSS2. TMPRSS2 is a protease which cleaves the spike protein into its S1 and S2 subunits to allow the virus to attach to ACE2 and enter the cell. When the genome of the pseudovirus particles integrates into the host cell genome after entry into cells, firefly luciferase expression is proportional to the number of cells that were transduced. After 48 hrs incubation, the cells are lysed and comparison between the luciferase signals detected in cells only, in cells transduced with pseudotype virus only, and in cells transduced with pseudotype virus in the presence of antibodies, enables determination of neutralization activity against the pseudotype tested.

TABLE E4-1
Potency of antibodies in pseudovirus neutralisation assay

| | | IC50 | | Fold change |
|---|---|---|---|---|
| | IC50 | Confidence | IC50 | relative to SAD |
| Clone | (pM) | interval (pM) | (ug/ml) | S35 antibody |
| IMPI-059 | 3.2 | 2.38 to 4.24 | 0.0005 | 1407.84 |
| IMPI-017 | 9.0 | 6.64 to 12.1 | 0.0013 | 501.75 |
| IMPI-004 | 34.0 | 25.7 to 44.8 | 0.0051 | 132.43 |
| IMPI-055 | 36.2 | 21.5 to 57.8 | 0.0054 | 124.56 |
| IMPI-029 | 43.3 | 28 to 66.31 | 0.0065 | 104.11 |
| IMPI-056 | 82.2 | 48.4 to 137 | 0.0123 | 54.88 |
| IMPI-047 | 87.2 | 38.5 to 186 | 0.0131 | 51.69 |
| IMPI-054 | 94.5 | 42.5 to 194 | 0.0142 | 47.71 |
| IMPI-021 | 164.2 | 95 to 274 | 0.0246 | 24.28 |
| IMPI-002 | 343.9 | 113 to 895 | 0.0516 | 11.59 |
| IMPI-052 | 507.7 | 254 to 942 | 0.0761 | 7.85 |
| IMPI-005 | 530.5 | 308 to 889 | 0.0796 | 8.50 |
| IMPI-042 | 1441.5 | 727 to 2690 | 0.2162 | 2.77 |
| IMPI-013 | 2305.5 | 1070 to 4340 | 0.3458 | 1.73 |
| IMPI-028 | 2921.4 | 1040 to 8370 | 0.4382 | 1.36 |
| IMPI-060 | 6168.9 | 2440 to 15200 | 0.9253 | 0.73 |
| IMPI-024 | 6846.2 | 2410 to 31700 | 1.0269 | 0.58 |
| IMPI-063 | 9981.9 | 2330 to 270000 | 1.4973 | 0.35 |
| IMPI-068 | 13091.4 | 2100 to 1540000 | 1.9637 | 0.26 |
| IMPI-032 | 14862.7 | 6370 to 60500 | 2.2294 | 0.27 |
| IMPI-037 | 30672.1 | 7300 to 1200000 | 4.6008 | 0.15 |
| IMPI-027 | 31645.6 | 5320 to 3060000 | 4.7468 | 0.13 |
| IMPI-038 | 54979.4 | 13400 to 3020000 | 8.2469 | 0.07 |
| IMPI-067 | 72329.5 | 13600 to 1990000 | 10.849 | 0.05 |
| IMPI-022 | n/a | n/a | n/a | n/a |
| IMPI-006 | n/a | n/a | n/a | n/a |
| IMPI-033 | n/a | n/a | n/a | n/a |
| mAb A | 34.404 | 24.1 to 48.3 | 0.0052 | 151.72 |
| mAb B | 291.3 | 125 to 649 | 0.0437 | 17.92 |
| mAb C | 227.4 | 159 to 322 | 0.0341 | 22.96 |
| mAb D | n/a | n/a | n/a | n/a |
| 4A8 | 960.4 | 444 to 2200 | 0.1441 | 5.44 |
| SAD S35 | 5220 | 2770 to 9910 | 0.7830 | 1 |

Neutralisation potency of selected antibodies against SARS CoV2 pseudotype virus expressed as IC50 values in picomolar. Confidence intervals are indicated, and data expressed as fold change relative to SAD S35 antibody. n/a indicates antibody did not reach 50% neutralisation and IC50 cannot be calculated.

### Materials and Methods

### Generation of SARS-CoV-2 Recombinant Pseudotype Virus

Non-replicative pseudoparticles were generated using plasmids obtained from Dr Nigel Temperton, University of Kent (Hyseni et al Viruses 12(9):1011 2020). Plasmid pCAGGS-ACE2 contains the human ACE2 encoding sequence (GenBank: AB193259.1) in the expression vector pCAGGS. Plasmid pCAGGS-TMPRSS2 contains the human TMPRSS2 encoding sequence (NCBI Reference Sequence: NM_001135099.1) in the expression vector pCAGGS. Briefly, a total of 5×10^{6 Lenti-X} 293T cells (Clontech, 632180) were seeded overnight in 10 cm dishes in DMEM media containing 10% heat-inactivated fetal bovine serum (Gibco). The following day, the cells were transfected with 1 g of the spike expression plasmid, pcDNA 3.1-SARS2-spike, lg gag-pol (p8.9), and 1.5 g CSFLW (a DNA plasmid encoding lentivirus backbone with firefly luciferase as a reporter gene) using the lipofectamine Fugene (Promega cat #E2311) according to manufacturer's instructions. Supernatant containing pseudoparticles was collected at 48, 72 and 96h post transfection and sterile filtered using 0.45 uM cellulose acetate filters. Supernatant were either aliquoted and stored at −80° C. or concentrated further. If concentrating, supernatants were centrifuged at 6000g overnight and then the filtered supernatant was pooled in the desired volume and frozen at −80° C./liquid nitrogen. TCID50 (50% endpoint titre) of the viral pseudoparticles was then calculated using serial dilution according to Spearman-Karber method.

pCSFLW is the firefly luciferase reporter-expressing lentivirus-backbone plasmid, which produces the lentivirus modified RNA genome with a long terminal repeat, packaging signal, promoter firefly luciferase reporter gene. The pCSFLW is derived from pCSGW where the green fluorescent protein encoding gene is replaced by the firefly luciferase reporter gene.

### Target Cell Line

Lenti-X HEK293T cells expressing ACE2 and TMPRRS2 were used as the target cell for the pseudotype neutralisation assay. Cells were prepared 24 hours in advance for the assay. A total of 2.5×106 Lenti-X cells were seeded overnight in a T25 flask in DMEM media containing 10% heat-inactivated fetal bovine serum (Gibco). The following day the cells were transfected with 1 μg pCAGGS-ACE2 plasmid and 75 ng pCAGGS-TMPRSS2 plasmid using Fugene (Promega cat #E2311) transfection reagent according to manufacturer's instructions. After 24 hrs, cells were removed by trypsinisation and used for the pseudovirus neutralisation assay.

### 8-Point Titration Pseudovirus Neutralization Assay

The purified antibodies were initially diluted 5-fold in DMEM media containing 10% heat-inactivated fetal bovine serum (Gibco) in a 96-well plate. Antibodies were then further titrated into a 384 well plate at 8-point dilution in triplicates starting from 50 nM concentration to reach a final concentration of 0.64 pM. Positive (Sino Biologicals 40150-D001 and Acro Biosystems SAD-S35 monoclonal antibodies) and negative (human IgG1 antibody, in house) control antibodies were also diluted at the same concentrations. 15 μL/well of titrated antibodies were plated into 384 well plates. The following controls were used: cells only, cells and virus (no antibody), positive control antibody, negative control antibody. 15 μL/well of diluted pseudotyped virus was then added (except to the cells only control) at a concentration of 50-100 TCID₅₀. The plate was centrifuged at 500 rpm for 5 sees and the antibodies and pseudovirus left to incubate for 1 hour at 37° C. (5% CO₂). Lenti-X 293T cells transiently expressing recombinant human ACE2 and TMPRSS2 were then added to each well to obtain a final cell number of 5×10³ cells per well. The plate was centrifuged at 500 rpm for 5secs and left to incubate for 48 hours at 37° C. (5% CO₂). 35 ul of BrightGlo (Promega cat #E2610) was added to each well as the detection reagent. The plate was read on an Envision plate reader (Perkin Elmer) using X nm for excitation and detecting luminescence at x and x nm. The percent neutralization of each antibody was calculated based on the luciferase activity, normalised to cells only (100%) and virus/cells only (no antibody) as 0%.

### Example 5. Sars-Cov-2 Wild-Type Live Virus Neutralization Assay

The ability of antibodies to neutralize entry of wild type SARS-CoV-2 virus was assessed by neutralization assay on Vero-E6 cells. Antibody titrations were incubated with 100 TCID50 of SARS-CoV-2/England/IC19/2020 strain of virus (from Imperial College London) and incubated with Vero E6 cells for 5 days. Results for a selection of antibodies are presented in Table E5-1, expressed as the lowest dilution (as a pM concentration) that showed 100% virus neutralisation.

TABLE E5-1
Potency of antibodies in live virus neutralisation assay

| | SARS COV2 |
|---|---|
| | neutralisation |
| | (complete virus |
| Clone | inhibition, pM) |
| IMPI-004 | 391 |
| IMPI-029 | 781 |
| IMPI-056 | 781 |
| IMPI-047 | 781 |
| IMPI-005 | 1563 |
| IMPI-006 | 25000 |
| IMPI-055 | 391 |
| IMPI-054 | 391 |
| IMPI-017 | 781 |
| IMPI-059 | 1563 |
| IMPI-037 | 25000 |
| IMPI-060 | 12500 |
| IMPI-013 | 50000 |
| IMPI-028 | 12500 |

Neutralisation potency of selected antibodies against wild type SARS CoV2 virus expressed as complete virus inhibition values in picomolar.

We found excellent correlation between performance of anti-RBD antibodies in the pseudovirus assay and live virus assay, indicating that the pseudovirus neutralisation assay is a good surrogate for measuring inhibition of viral entry to cells. Potent activity in this assay indicates these antibodies may neutralise the entry of the virus into cells in vivo.

### Materials and Methods

SARS-CoV-2/England/IC19/2020 was isolated on Caco2 cells from a clinical sample collected from a patient admitted to St. Mary's Hospital in London, United Kingdom. Antibodies were serially diluted (from a starting concentration of 0.5-luM) in assay diluent consisting of DMEM (Gibco, Thermo Fisher Scientific) with 1% penicillin-streptomycin (Thermo Fisher Scientific), 0.3% BSA fraction V (Thermo Fisher Scientific) and 0.25 μg mL-1 TPCK trypsin (Worthington). Antibody dilutions were incubated with 100 TCID50 per well of SARS-CoV-2/England/IC19/2020 diluted in assay diluent for 1 h at RT and transferred to 96-well plates pre-seeded with Vero-E6 cells. A TCID50 (tissue culture infectious dose for 50% infection) is the amount of virus able to infect 50% of tissue culture cells in wells (i.e. 2/4, 4/8, 6/12) at a given dilution value of the virus). 100 TCID50s is therefore 100 times the TCID50. Antibody dilutions were performed in duplicate. Plates were incubated at 37° C., 5% C02 for 5 days before adding an equal volume of 2X crystal violet stain to wells for 1 h. Plates were washed, wells were scored for cytopathic effect and a 100% neutralization titre calculated as the highest antibody dilution at which full virus neutralization (no evidence of cell infection) occurred. As the starting antibody concentration is known, then the antibody dilution that resulted in complete virus inhibition is reported the concentration of antibody that results in 100% or complete virus neutralisation.

### Example 6. Surface Plasmon Resonance (Spr) Determination of Binding Affinity and Kinetics

SPR runs (single cycle kinetics) were carried out using Biacore 8K (Cytiva) on a chip (CM4 from Cytiva) with anti hFc antibody immobilised on the chip (Human antibody capture kit; Cytiva). The chip is a dextran-coated layer of gold, and the anti-hFc is attached using amine coupling. Buffer HBS-P+(Cytiva) was used as a running buffer. This buffer is at pH 7.4 and comprises 0.01 M HEPES (4-(2-hydroxyethyl)-1-piperazineethanesulfonic acid, 0.15 M NaCl and 0.05% v/v surfactant P20 in aqueous solution). Chip temperature was maintained at 25 degrees C.

IMPI antibodies were produced as human IgG1 by expression in CHO cells and were purified as previously described. Antibodies were captured at 1 ug/ml. Receptor binding domain (Neil King, University of Washington), S1 spike domain (Sino Biological),S1 spike domain D614G variant (Sino Biological), S2 spike domain (Sino Biological) or spike protein trimer (produced in house, either wild type as shown in FIG. 2A or containing PP mutation as shown in FIG. 2B3, and in both cases containing C-terminal trimerisation domain) were injected at 0.39, 1.56, 6.25, 25 and 100 nM for 120 s at 30 ul/min. Dissociation was monitored for 600 s. Chip surface was regenerated with 3M Magnesium Chloride. Reference and blank subtracted sensorgrams were fitted using 1:1 binding model (Biacore Insight Evaluation Software).

TABLE E6-1
Kinetic constants for isolated RBD measured by SPR

| | | | kD binding to |
|---|---|---|---|
| Clone | kₐ (1/Ms) | k_{d} (1/s) | RBD (M) |
| IMPI-004 | 2.01E+06 | 6.87E−04 | 3.41E−10 |
| IMPI-029 | 8.08E+05 | 1.57E−04 | 1.95E−10 |
| IMPI-056 | 5.84E+05 | 2.57E−04 | 4.40E−10 |
| IMPI-047 | 3.68E+05 | 8.78E−04 | 2.38E−09 |
| IMPI-005 | 2.30E+06 | 7.10E−03 | 3.09E−09 |
| IMPI-006 | 9.30E+09 | 8.19E+00 | 8.80E−10 |
| IMPI-055 | 2.75E+06 | 2.64E−03 | 9.59E−10 |
| IMPI-054 | 5.64E+05 | 6.27E−04 | 1.11E−09 |
| IMPI-017 | 5.22E+05 | 9.76E−04 | 1.87E−09 |
| IMPI-059 | 6.59E+06 | 4.26E−03 | 6.46E−10 |
| IMPI-037 | 2.50E+06 | 2.32E−02 | 9.28E−09 |
| IMPI-060 | 4.69E+05 | 2.77E−02 | 5.92E−08 |
| IMPI-013* | | | No binding |
| IMPI-028 | 5.34E+05 | 1.36E−02 | 2.56E−08 |
| IMPI-027 | 6.48E+09 | 2.63E+01 | 4.06E−09 |
| IMPI-033 | 2.20E+06 | 7.41E−03 | 3.37E−09 |
| IMPI-021 | 5.48E+05 | 4.43E−03 | 8.08E−09 |
| IMPI-032** | | | No binding |
| IMPI-038 | 4.20E+06 | 1.23E−02 | 2.92E−09 |
| IMPI-022** | | | No binding |
| IMPI-024 | 2.12E+06 | 1.34E−03 | 6.33E−10 |
| IMPI-002 | 1.82E+06 | 4.02E−02 | 2.21E−08 |
| IMPI-052 | 1.93E+06 | 2.54E−02 | 1.32E−08 |
| IMPI-042 | 5.36E+05 | 6.17E−04 | 1.15E−09 |
| IMPI-063* | | | No binding |
| IMPI-067** | | | No binding |
| IMPI-068 | 1.85E+06 | 7.50E−02 | 4.05E−08 |
| mAb A | 4.89E+06 | 4.00E−03 | 8.19E−10 |
| mAb B | 3.65E+06 | 3.31E−02 | 9.06E−09 |
| mAb C | 2.93E+06 | 3.39E−02 | 1.16E−08 |

Kₐ Association rate constant
K_{d} Dissociation rate constant
K_{D} Equilibrium dissociation constant
*IMPI-013 and IMPI-063 are S2 binders so this lack of RBD binding is as expected.
**IMPI-022, IMPI-032 and IMPI-067 are trimer-only binders which did not exhibit detectable binding to the isolated RBD by SPR.

Comparing the SPR data for the ACE-2 competing anti-RBD antibodies with the corresponding data for these antibodies in the pseudovirus neutralisation assays, we see that the antibodies which showed the most potent (<100 pM) neutralisation in the pseudoviral neutralisation assay also showed high affinity (sub nM KD, i.e., <1E09 M) as determined by SPR. This includes IMPI-059, IMPI-004, IMPI-029, IMPI-056, IMPI-006 and IMPI-055.

A selection of the best antibodies from the ACE-2 competing anti-RBD group were further assessed by SPR for binding to the full S1 subunit, S2 subunit and to the spike protein trimer.

In summary we found higher affinity binding to RBD compared with reference antibodies, no significant binding to S2. The IMPI antibodies all bound a mutant of the S1 subunit of the spike protein, D614G. D614G is located in the S 1 subunit downstream of the RBD. In the trimer protein this residue is positioned close to the interface with the S2 subunit. Despite its distance from the RBD, residue 614 does appear to be capable of influencing binding of at least some anti-RBD antibodies. Reference antibody mAb B, an ACE-2 competing anti-RBD antibody which binds to the side of the RBD domain, was observed to show a decrease in affinity to D614G. The D614G variant is a mutation that arose in the virus, observed near the beginning of the epidemic, which has gradually arisen to become the dominant variant of the virus. The significance of D614G is unclear but in some in vitro data suggests that this mutation makes the virus slightly more infectious, with more viral shedding, and since February 2020 this strain has become the most widespread form of the virus around the world. Therefore, binding to the spike protein comprising the D614G mutation is highly advantageous in antibodies intended for therapeutic use, as well as in diagnostics for detecting whether a sample is positive for SARS-CoV-2. Retention of binding to D614G strain by the IMPI antibodies is therefore encouraging for their use in the clinic.

TABLE E6-2
Kinetic constants measured by SPR for S2 binding
neutralising antibodies

| Clone | Analyte | kₐ (1/Ms) | k_{d} (1/s) | KD (M) |
|---|---|---|---|---|
| IMPI-013 | Trimer (wt) | 4.40E+04 | 2.95E−05 | 6.70E−10 |
| IMPI-013 | Stabilized trimer | 5.74E+04 | 1.72E−05 | 2.99E−10 |
| | (PP mutation) | | | |
| IMPI-013 | S2 | 9.33E+04 | 1.18E−05 | 1.26E−10 |
| IMPI-013 | S1 | No binding | | |
| IMPI-013 | RBD | No binding | | |
| IMPI-063 | Trimer (wt) | 4.11E+05 | 9.39E−04 | 2.29E−09 |
| IMPI-063 | S2 | 9.19E+09 | 7.77E+00 | 8.45E−10 |
| IMPI-063 | RBD | No binding | | |

The S2 binder IMPI-013 was highly specific for binding to the S2 domain. High affinity binding to the trimer (both wild type sequence and stabilised PP mutant form) and to the isolated S2 domain was observed for IMPI-013. IMPI-013 did not show detectable binding to either the full isolated S1 subunit or to the isolated RBD. It is notable that IMPI-013 shows a very low dissociation rate. Impressively, observation of the sensorgram showed that the majority of IMPI-013 remained associated with the S2 domain or trimer for a long time after binding, during a 10 minute dissociation period.

A second S2 binder, IMPI-063, also showed high specificity for binding to the S2 domain and bound to the wild type spike trimer protein, but showed no detectable binding to the isolated RBD domain. SPR was not performed for IMPI-063 with stabilised spike trimer or S1 domain.

### Example 7. Epitope Binning

For competition studies, a sandwich method on the CM4 chip with anti hFc antibody immobilised on the chip was used (i.e., same chip as used in Example 6). First, purified CHO-expressed human IgG1 antibody at 1 μg/mL was captured for 240 s at 10 μL/min. Chip surface was then blocked with 800 nM irrelevant control human antibody for 180 s at 30 μL/min, to block remaining Fc binding sites on the chip surface. Receptor binding domain was injected at 200 nM for 120 s at 10 uL/min. A second antibody was then injected at 200 nM for 150 s at 10 μL/min. Reference cell without the first antibody captured was used for subtraction. Sensorgrams were analysed using Biacore Insight Evaluation Software.

A selection of anti-RBD ACE-2 competitor IMPI antibodies were tested for inter-competition. Antibody IMPI-037 did not compete with the other antibodies tested. This antibody also showed relatively low potency in functional assays as described above. This antibody thus represents one “bin”. A second “bin” is represented by IMPI-006, as this competed with all tested antibodies except IMPI-004, IMPI-055 and IMPI-059. A third “bin” is represented by all other antibodies, which all competed with one another.

Antibody IMPI-037 is of particular interest in the present invention.

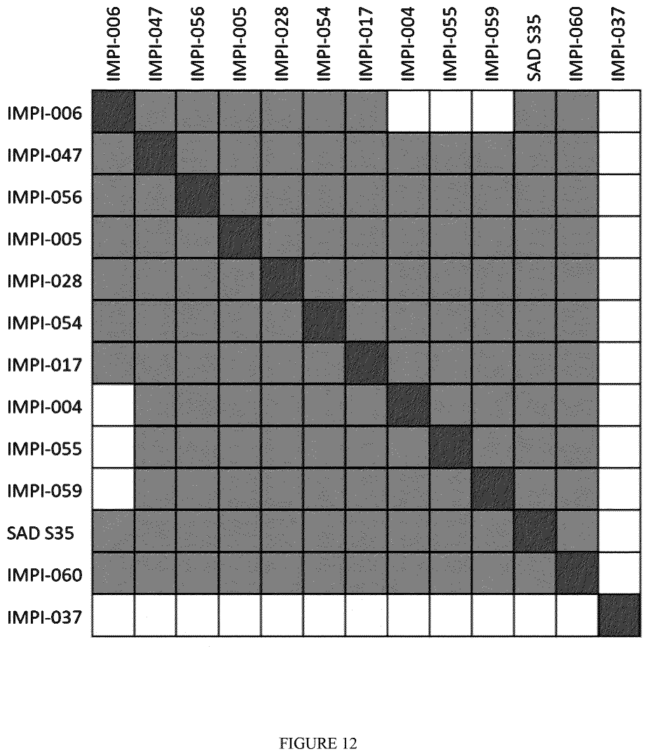

FIG. 12 shows the results of epitope binning competition study.

### Example 8. Spr Determination of Competition with Ace2 for Binding Spike Protein RBD

The ability of antibodies to compete with human ACE-2 for binding to RBD was determined by SPR using the protocol described above in Example 7, using a human ACE2-Fc fusion protein in place of the test antibody.

TABLE E8-1
SPR results for competition of antibodies
with ACE2 for binding to RBD.

| | RBD:ACE-2 competition |
|---|---|
| IMPI-004 | yes |
| IMPI-029 | yes |
| IMPI-056 | yes |
| IMPI-047 | yes |
| IMPI-005 | yes |
| IMPI-006 | yes |
| IMPI-055 | yes |
| IMPI-054 | yes |
| IMPI-017 | yes |
| IMPI-059 | yes |
| IMPI-037 | yes |
| IMPI-060 | yes |
| IMPI-028 | yes |
| IMPI-027 | no |
| IMPI-033 | no |
| IMPI-021 | yes |
| IMPI-038 | no |
| IMPI-024 | no |
| IMPI-002 | yes |
| IMPI-052 | yes |
| IMPI-042 | yes |
| IMPI-068 | no |
| SAD S35 | yes |

In general, the preliminary results from the HTRF study (Example 3) were confirmed. IMPI-004, IMPI-029, IMPI-056, IMPI-047, IMPI-005, IMPI-055, IMPI-054, IMPI-017, IMPI-059, IMPI-037, IMPI-021, IMPI-002, IMPI-052 and IMPI-042 were all confirmed to compete with ACE2 in both types of assay. Additionally, antibody IMPI-028 exhibited competition with human ACE-2 for binding to the RBD as determined by SPR.

### Example 9. Sars-Cov-2 Wild-Type Virus Neutralisation Assay by Foci-Reduction Assessment

Antibodies IMPI-004, IMPI-013, IMPI-017 and IMPI-059 were compared against reference mAbs COV2-2196 and COV2-2130 (Zost, S. J. et al. Potently neutralizing and protective human antibodies against SARS-CoV-2. Nature 2020 doi.org/10.1038/s41586-020-2548-6) in a neutralisation assay with live SARS-CoV-2. These two control mAbs COV2-2196 and COV2-2130 are presently (October 2020) clinical candidate therapeutic antibodies.

IMPI-059 was the most potent antibody in this assay, with an IC50 of 26 pM.

IMPI-004 had an IC50 of 13 ng/ml (86 pM).

IMPI-017 had an IC50 of 49 ng/ml (326 pM).

The S2 subunit binder, IMPI-013, had an TC50 of 3400 pM (3.4 nM).

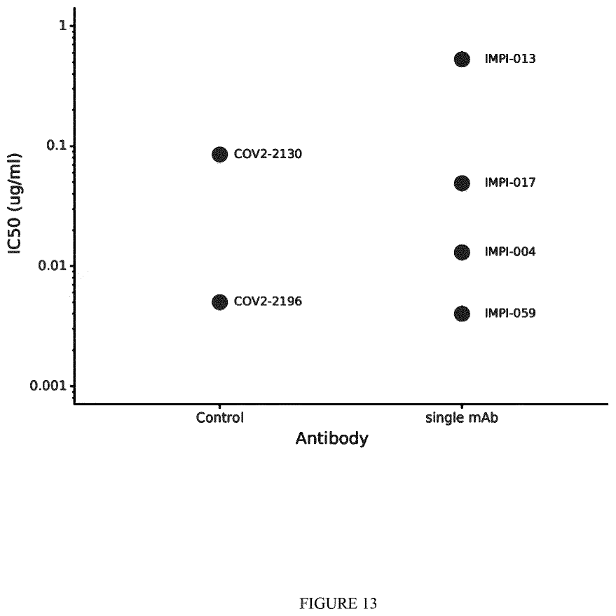

FIG. 13 presents these results with a log scale of IC50.

In general, the data obtained in this live virus assay (complete neutralisation or IC50 values) compared well with those from the pseudovirus neutralisation assay. Similar potent neutralisation profiles were observed, although absolute IC50 values differed as would be expected.

Based on the data presented herein overall, IMPI-059 represents the strongest candidate antibody based on its performance as a monoclonal composition. This is an anti-RBD, ACE2 neutralising antibody. IMPI-004 may represent the next strongest choice in this category, again based on its performance as a monoclonal composition. IMPI-017 is an anti-RBD, ACE2 neutralising antibody with high potency in the pseudovirus assay and good performance in the 100% neutralisation live virus assay, suggesting it is an interesting antibody. The S2 binder, IMPI-013, is also of particular interest in view of its different mode of binding and activity, coupled with high specificity and its neutralising ability. Combinations of these antibodies could be potent beyond additivity.

### Materials & Methods:

For this foci (plaque) reduction neutralization assay, tests were performed using passage 4 of SARS-CoV-2 Victoria/01/2020 (Caly et al Med. J. Aust. 212, 459-462 2020). A virus suspension was added at an appropriate concentration to yield approximately 100 foci in a final assay well in DMEM containing 1% FBS (D1). Virus suspension at appropriate concentrations in D1 (60 l) was mixed with antibody (60 l) to give a final concentration of 10 μg ml—1, 2.5 μg ml—1, 0.625 μg ml—1, 0.156 μg ml—1, 0.039 μg ml—1, 0.0097 μg ml—1, 0.0024 μg ml—1, 0.00061 μg ml—1, 0.00015 μg ml—1, 0.000038 μg ml—1, and 0.0000095 g ml—1, and without antibody as the 100% control well in triplicate, in wells of a 24-well tissue culture plate, and incubated at room temperature for 30 min. Thereafter, 50 μl of antibody/virus complexes were added into Vero cells monolayer, in duplicate, in wells of a 96-well tissue culture plate incubated for 2 h at 37° C. before being overlain with 0.5 ml of D1 supplemented with carboxymethyl cellulose (1.5%). Cultures were incubated for a further 24 hours at 37° C. before foci were revealed by staining with anti-NP followed by anti-human IgG-HRP. NP (nucleoprotein) is a SARS-CoV2 specific protein that is present in infected cells and therefore a good antibody marker for detecting infected cells. TrueBlue peroxidase substrate was added and stained foci of infected cells were identified (stained foci of infected cells should be clearly visible in 5 min). The IC50 was calculated by assessing the number of foci plaques in the 100% control well and the reduction in foci numbers corresponding to each antibody concentration. The results can be analysed manually or by standard curve fitting methods allowing the interpolation or calculation of the antibody concentration required to cause a 50% decrease in the number of foci present in the wells (TC50).

### Example 10. Classification of RBD Binding Antibodies into Epitope Communities

In contrast to previous studies that classified mAbs using germline or structural information the RBD-reactive mAbs analysed here were instead distinguished by a competition profile created by high-throughput surface plasmon resonance (HT-SPR, Carterra). RBD-directed antibodies can be sorted into seven core “communities” (as described in Hastie et al., 2021), that are broadly defined by the competition profiles of each mAb to one another. Communities can be further divided into finer clusters and bins based on their discrete competition with other clusters and/or their ability to compete with ACE2. Twenty two IMPI antibodies were assigned to epitope communities (Table E10-1). Fifteen antibodies were assigned to epitope community RBD-2, six antibodies to RBD-5 and one to RBD-6 (IMPI-006) based on their epitope competition profiles.

To understand the position of each epitope community relative to the others negative stain electron microscopy was performed for representative antibodies across the different classes to determine the binding footprint on the Spike protein. The antibodies described here fall into three different classes. RBD-2 (community 2) mAbs compete with ACE2 and generally require the RBD to be in the “up” conformation for binding. The binding site for Group 2 mAbs is shifted from the centre of the ACE2 binding site towards the peak of the receptor binding motif. Most RBD-2 mAbs bind bivalently to a single spike trimer (in contrast to other mAbs that cross link multiple Spike trimers).

The epitope community is also characterised by the propensity of particular Spike mutations to escape antibody-mediated neutralization. Several new SARS-CoV-2 strains have continued to emerge from late 2020 and into 2021, some of which have been designed as variants of concern (VOC) by the World Health Organisation (WHO). The currently VOC strains listed by the WHO are: Alpha (B.1.1.7), Beta (B.1.351), Gamma (P.1), and Delta (B.1.617.2) strains. The Beta strain (also known as B.1.351 lineage) originated in South Africa and contains several mutations which may impact the binding of antibodies and vaccines. All antibodies were tested for neutralization using WA-1 strain and the VOC Beta/B.1.351. Most of the antibodies in RBD-2 were impacted by the mutations present in the Beta strain and showed a decrease in neutralizing potency to this strain (Table E10-1).

Antibodies in class 5 (RBD-5) bind to the outer face of the RBD and can do so in either the “up” or “down” configuration without steric hindrance. RBD-5 mAbs bind away from the receptor binding motif and toward the binding site for the S309 antibody and do not block ACE-2. RBD-5 mAbs that were neutralising often mediated cross-linking of Spike proteins (Hastie et el., 2021) which may provide a possible mechanism of neutralisation for these antibodies that do not block ACE-2. RBD-5 mAbs also showed broad resistance to nearly all virus mutations analysed in Hastie et al, 2021, indicating that these mAbs may be more useful clinically as they have wider breadth of response across different variant strains. Antibody IMPI-037 is particularly interesting as this retains potent neutralisation of the Beta variant strain.

Antibodies inthe RBD-6 class (IMPI-006) block ACE2 and bindto the inner face ofthe RBD. They require two RBDs (the binding RBD and adjacent RBD)to be inthe “up” configuration. Duetothe binding location away from the receptor binding motif, RBD-6 antibodies are also resistant to mutations in VOCs (Hastie et al., 2021), which makes them attactive as broad therapeutics.

Classification of antibodies into different epitope communities is based on their epitope binding site and could be important for identifying mAbs that could be combined together as cocktails and identifies those mAbs that are most likely to be resistant to virus strain variation.

TABLE E10-1
Assay data used to categorise antibodies into different communities

| | | SPR | SPR | Live virus | Live virus | RBD |
|---|---|---|---|---|---|---|
| | ACE2 | binding | binding | WA-1 strain | B.1.351 | binding |
| | blocking | to RBD | to NTD | IC50 | strain IC50 | epitope |
| Clone ID | (%) | (kD, M) | (kD, M) | (ng/ml) | (ng/ml) | community |
| IMPI-002 | 100 | 3.38E−08 | | 17 | 15670 | 2 |
| IMPI-004 | 99.88 | 8.42E−10 | 6.53E−07 | 0.32 | nt | 2 |
| IMPI-005 | 100 | 2.49E−08 | | 11 | 25000 | 2 |
| IMPI-006 | 96.76 | 1.91E−07 | | nt | 25000 | 6 |
| IMPI-013 | 2.43 | | | 190 | nt | nd (not RBD) |
| IMPI-017 | 97 | 2.25E−09 | 1.05E−06 | 10 | nt | 2 |
| IMPI-021 | 99.62 | 1.47E−08 | | 3 | 25000 | 2 |
| IMPI-022 | | | 6.03E−07 | 18 | 1609 | (not RBD) |
| IMPI-024 | 17.44 | 1.16E−09 | 6.60E−07 | 1001 | 1241 | 5 |
| IMPI-027 | 46.24 | 6.31E−09 | 2.91E−06 | 1817 | 5398 | 5 |
| IMPI-028 | 100 | 1.62E−07 | | 66 | 7435 | 2 |
| IMPI-029 | 97.91 | 4.46E−10 | 2.19E−07 | 2 | 25000 | 2 |
| IMPI-032 | | | | 7520 | nt | nd (not RBD) |
| IMPI-033 | 31.24 | 3.02E−09 | 1.82E−06 | 3671 | 8896 | 5 |
| IMPI-037 | 100 | 2.23E−08 | | 12 | 0.38 | 5 |
| IMPI-038 | 32.21 | 8.26E−10 | 4.75E−07 | 223 | 5894 | 5 |
| IMPI-042 | 99.26 | 1.87E−09 | 6.62E−07 | 12 | 25000 | 2 |
| IMPI-047 | 98.22 | 6.65E−09 | 2.32E−06 | 12 | 25000 | 2 |
| IMPI-052 | 100 | 2.41E−08 | | 10 | 3695 | 2 |
| IMPI-054 | 99.42 | 2.06E−09 | 6.02E−07 | 15 | 25000 | 2 |
| IMPI-055 | 100 | 1.85E−09 | 1.36E−06 | 0.32 | 25000 | 2 |
| IMPI-056 | 99.55 | 1.59E−09 | 6.82E−07 | 3 | 25000 | 2 |
| IMPI-059 | 100 | 2.30E−09 | 1.65E−06 | 0.32 | 3935 | 2 |
| IMPI-060 | 100 | 1.41E−07 | | 171 | nt | 2 |
| IMPI-063 | 4.46 | | | 10399 | nt | nd (not RBD) |
| IMPI-067 | | | 8.45E−08 | 2 | 25000 | nd (not RBD) |
| IMPI-068 | 33.87 | 9.47E−09 | 2.57E−06 | 2541 | 4507 | 5 |

K_{D} : Equilibrium dissociation constant.
nt: not tested. For the epitope community determination these antibodies (IMPI-067, IMPI-063, IMPI-022, IMPI-032, IMPI-013) were not RBD binding and could not be assigned to the RBD binding bins.

### Materials and Methods

### High-Throughput SPR Binding Kinetics

The binding kinetics measurements were performed on the Carterra LSA platform using HC30M sensor chips (Carterra) at 25 C. Two microfluidic modules, a 96-channel print-head (96PH) and a single flow cell (SFC), were used to deliver liquids onto the sensor chip. In each assay, a single analyte was titrated against multiple CoVIC antibody constructs. Full details are described in Hastie et al., 2021.

Goat anti-Human IgG Fc secondary antibody was first immobilized onto the chip through amine-coupling. Briefly, the chip was first activated by 100 mM N-Hydroxysuccinimide (NHS) and 400 mM 1-Ethyl-3-(3-dimethylaminopropyl) carbodiimide hydrochloride (EDC) (GE healthcare, mixed 1:1:1 with 0.1 M MES buffer at pH 5.5) for 600 seconds, followed by immobilization of anti-Hu IgG Fc (in 10 mM Sodium Acetate at pH 0.5) at 50 g/ml for 900 seconds. Unreactive esters were quenched with a 600-second injection of 1 M ethanolamine-HCl at pH 8.5. The chip was then exposed to double pulses (30 seconds per pulse) of 10 mM Glycine at pH 2.0. The IgG antibodies were then captured by the anti-Hu IgG Fc at 5 g/ml for 600 seconds using the 96PH, with 1X HBSTE buffer (10 mM HEPES pH 7.4, 150 mM NaCl, 3 mM EDTA and 0.01% Tween-20) as running buffer and antibody diluent. Each antibody construct at a given diluted concentration was immobilized onto 8 separate spots of the same chip, enabling replicating binding kinetics measurements.

A two-fold dilution series of the antigen was prepared in 1×HBSTE buffer. The top concentration for RBD, NTD and D614-HexaPro was respectively 40 μg/ml (1.11 M), 320 μg/ml (5.71 M) and 100 μg/ml (0.181 μM). A single antigen was used in each assay. The antigen at different concentrations was then injected using SFC onto the chip surface from the lowest to the highest concentration without regeneration, including several injections of buffer before the lowest non-zero concentration for signal stabilization. For each concentration, the data collection time-length for 42 baseline, association and dissociation were 120 seconds, 300 seconds and 900 seconds, respectively. For all assays the running buffer for titration was 1× HBSTE. The titration data collected were first pre-processed in the NextGenKIT (Carterra) software, including reference subtraction, buffer subtraction and data smoothing. The data were then exported and analyzed using the TitrationAnalysis tool. The RBD, NTD and D614-HexaPro binding time courses for each antibody construct immobilized on different spots were fitted to a 1:1 Langmuir model to derive ka, kd and KD values.

### High-Throughput SPR Epitope Binning

Epitope binning was performed with a classical sandwich assay format on a Carterra LSA®HT-SPR instrument equipped with a CMDP sensor chip at 25° C. and in a HBSTE-BSA running buffer (10 mM HEPES pH 7.4, 150 mM NaCl, 3 mM EDTA, 0.05% Tween-20, supplemented with 0.5 mg/ml BSA). Two microfluidic modules, a 96-channel print-head (96PH) and a single flow cell (SFC), were used to deliver samples onto the sensor chip. Surface preparation was performed with 25 mM MES pH 5.5 with 0.05% Tween-20 as a running buffer. The chip was activated with a freshly prepared solution of 130 mM 1-ethyl-3-(3-dimethylaminopropyl)carbodiimide (EDC)+33 mM N-hydroxysulfosuccinimide (Sulfo-NHS) in 0.1 M MES pH 5.5 using the SFC. Antibodies were immobilized using the 96PH for 10 minutes at 10 μg/mL diluted into 10 mM sodium acetate (pH 4.25). Unreactive esters were quenched with a 7-minute injection of 1 M ethanolamine-HCl (pH 8.5) using the SFC. The binning analysis was performed over this array with the HBSTE-BSA buffer as the running buffer and sample diluent. The RBD antigen was injected in each cycle for 4 minutes at 50 nM (1.8 μg/mL) and followed immediately by a 4-minute injection of the analyte antibody at 30 μg/mL (200 nM for IgG constructs). The surface was regenerated each cycle with double pulses (17 seconds per pulse) of 10 mM Glycine pH 2.0. Data was processed and analyzed with Epitope®768 software (Carterra).

### Negative-Stain EM to Define Antibody Binding Area

Full details are described in Hastie et al., 2021. Fabs were obtained for EM study using either IdeS (Promega) or papain (Sigma), and purified by ion exchange chromatography using a MonoQ column (GE). Fab (70 μg) or IgG (140 μg); were incubated with 140 μg purified HexaPro. D614G Spike ectodomain in TBS buffer overnight at room temperature. The final concentration for Spike or IgG in incubation solution was −0.25 μg/L. Spike-antibody complexes were purified by SEC with a Superdex 6 Increase 10/300 column (GE) and verified by SDS-PAGE. For each complex, 4 μL of sample (˜0.02 mg/mL) was applied to a CF400-Cu negative-stain grid (Electron Microscopy Sciences), and stained with 0.75% uranyl formate (Electron Microscopy Sciences). Between 50 and 400 micrographs were collected for each sample using a Titan Halo electron microscope (Thermo Fisher) and a Falcon 3EC direct electron detector at the magnification of 58,000X. EM-map reconstruction was performed using CryoSPARC and the maps were aligned and displayed using Chimera X. Specifically, models having different RBD status (One RBD up: PDB:7A94; Two RBDs up: PDB:7DCX; and Three RBDs up: PDB:7K4N), were fitted into NS-EM maps for antibody binding area identification.

ACE2—blocking assay ACE2 blocking was measured using Biolayer Interferometry (BLI) on an Octet HTX instrument (Sartorius) by covalently immobilizing SARS-CoV-2 RBD and Human Serum Albumin (HSA) (reference to subtract response due to non-specific interactions) onto Amine Reactive 2nd Generation (AR2G) biosensors (Sartorius). The data was analyzed using Data Analysis HT 12.0 (CFR11) software (Sartorius). The biosensors were activated with a freshly prepared solution of EDC (1-Ethyl-3-[3-dimethylaminopropyl]carbodiimide hydrochloride) and s-NHS (N-hydroxysulfosuccinimide) in molecular biology grade water. RBD and HAS were diluted in 10 mM sodium acetate pH 5 buffer and immobilized onto 96 separate sensors to a loading density threshold not to exceed A, =0.7 nm.

Unreactive NHS esters on the surface of the sensors were quenched with 1M ethanolamine pH 8.5. Antibody and ACE2 binding were performed sequentially by dipping the RBD and HSA loaded biosensors into a well plate containing antibodies at 20 μg/ml followed by a solution of recombinant ACE2 (human IgGFc fused at 27.5 μg/ml for 5 minutes each. The diluent used for preparing antibodies and ACE2 solution was 1× kinetics buffer (Sartorius).

ACE2 binding to immobilized RBD was monitored in real time in the absence and presence of antibodies pre-bound to RBD. The CoVIC reference mAbs CC12.3 and CC12.14, and control SARS-CoV-2 Spike Neutralizing mAb (Sino Biological) were included in each experiment as a positive control. The percent ACE2 blocking was calculated as the percentage of decrease in ACE2 binding due to antibodies pre-bound to RBD compared with the ACE2 binding to RBD untreated with any antibody (1x kinetics buffer in place of antibody). The average of ACE2 binding to antibody untreated RBD was set as 0% blocking. The ACE2 blocking percentages shown for the CoVIC antibodies are the mean of triplicate measurements. For full details see Hastie et al., 2021.

Live virus neutralisation of variant strains Neutralization of authentic SARS-CoV-2 carrying D614G (WA-1 strain) and B.1.351 by mAbs was assessed using a method similar to that described in Hou et al. 2020. Under BSL-3 containment, serially-diluted mAbs at 8 concentrations are incubated with 800 PFU/well nLuc virus for one hour at 5% CO2 and 37° C. After incubation, the virus/antibody mixtures are added in duplicate to black-walled 96-well plates containing Vero E6/C1008 cells (2×104 cells/well). Each plate also contains virus-only control wells. The plates are incubated for 24 hr at 37° C., 5% CO2 and the cells are lysed before measurement of luciferase activity with the Nano-Glo Luciferase Assay System (Promega) according to the manufacturer's instructions. Neutralization activity is expressed as the concentration at which the observed relative light units (RLU) are reduced by 50% relative to virus-only control wells.

### REFERENCES

Hastie KM, Li H, Bedinger D, Schendel SL, Dennison SM, Li K, Rayaprolu V, Yu X, Mann C, Zandonatti M, Diaz Avalos R, Zyla D, Buck T, Hui S, Shaffer K, Hariharan C, Yin J, Olmedillas E, Enriquez A, Parekh D, Abraha M, Feeney E, Horn GQ; CoVIC-DB team1, Aldon Y, Ali H, Aracic S, Cobb RR, Federman RS, Fernandez JM, Glanville J, Green R, Grigoryan G, Lujan Hernandez AG, Ho DD, Huang KA, Ingraham J, Jiang W, Kellam P, Kim C, Kim M, Kim HM, Kong C, Krebs SJ, Lan F, Lang G, Lee S, Leung CL, Liu J, Lu Y, MacCamy A, McGuire AT, Palser AL, Rabbitts TH, Rikhtegaran Tehrani Z, Sajadi MM, Sanders RW, Sato AK, Schweizer L, Seo J, Shen B, Snitselaar JJ, Stamatatos L, Tan Y, Tomic MT, van Gils MJ, Youssef S, Yu J, Yuan TZ, Zhang Q, Peters B, Tomaras GD, Germann T, Saphire EO. Defining variant-resistant epitopes targeted by SARS-CoV-2 antibodies: A global consortium study. Science. 2021 Sep 23:eabh2315. doi: 10.1126/science.abh2315. Epub ahead of print. PMID: 34554826.
Hou YJ, Okuda K, Edwards CE, Martinez DR, Asakura T, Dinnon KH 3rd, Kato T, Lee RE, Yount BL, Mascenik™, Chen G, Olivier KN, Ghio A, Tse LV, Leist SR, Gralinski LE, Schafer A, Dang H, Gilmore R, Nakano S, Sun L, Fulcher ML, Livraghi-Butrico A, Nicely NI, Cameron M, Cameron C, Kelvin DJ, de Silva A, Margolis DM, Markmann A, Bartelt L, Zumwalt R, Martinez FJ, Salvatore SP, Borczuk A, Tata PR, Sontake V, Kimple A, Jaspers I, O'Neal WK, Randell SH, Boucher RC, Baric RS. SARS-CoV-2 Reverse Genetics Reveals a Variable Infection Gradient in the Respiratory Tract. Cell. 2020 Jul. 23; 182(2):429-446.e14. doi: 10.1016/j.cell.2020.05.042. Epub 2020 May 27. PMID: 32526206; PMCID: PMC7250779.

### Example 11: In Vivo Protection in K18 Hace2 Transgenic Mouse Model

A subset of IMPI antibodies were tested for in vivo activity against SARS-CoV-2 live virus using the K18 transgenic mouse model. These mice are engineered to express the entry receptor for SARS-CoV-2, human angiotensin converting enzyme 2 (hACE2), to make them susceptible to SARS-CoV-2 infection. The virus strain used for infection is SARS-CoV-2 USA-WA1/2020, which was isolated in the USA in January 2020 and serves as the SARS-CoV-2 reference strain for the United States.

Antibodies were administered 1 day prior to infection in groups of 10 mice per antibody. Results were expressed as the percentage of mice that survived at 10 days post infection. Control mice (injected with PBS) showed 0% survival.

Twelve antibodies showed activity in this assay (Table E 11-1) with survival ranging from 10% to 100%. Three antibodies showed particularly potent in vivo activity: IMPI-055 and IMPI-004 provided 80% protection and IMPI-037 showed 100% protection at 1.5 mg/ml. IMPI-037 is of special interest as this antibody shows strong neutralising activity in vivo and as described in Example 10 it is resistant to mutations present in the Beta virus strain. IMPI-037 is in epitope community RBD-5 (see Example 10) and other antibodies in this group may also have similar properties.

TABLE E11-1
In vivo activity of antibodies against SARS-COV-2 in
K18 hACE2 transgenic mouse model

| | In vivo K18 (0.5 mg/mL) | In vivo K18 (1.5 mg/mL) |
|---|---|---|
| Clone | % survival | % survival |
| IMPI-004 | 10 | 80 |
| IMPI-017 | nt | 30 |
| IMPI-021 | nt | 20 |
| IMPI-022 | 60 | nt |
| IMPI-029 | 50 | nt |
| IMPI-037 | nt | 100 |
| IMPI-054 | 10 | nt |
| IMPI-055 | 80 | nt |
| IMPI-056 | 20 | nt |
| IMPI-059 | 10 | nt |
| IMPI-063 | 20 | nt |
| IMPI-067 | 20 | 40 |

nt: not tested

### Materials and Methods

Experiments were performed at Texas Biomedical Research Institute, as described in Oladunni et al., 2020. Briefly, SARS-CoV-2, USA-WA1/2020 strain (Gen Bank: MN985325.1), was obtained from BEI Resources (NR-52281) and virus stocks prepared in Vero E6 cells (ATCC, CRL-1586). Virus stocks were titrated by standard plaque assays (PFU/ml) in Vero E6 cells and validated by using next generation sequencing. K18 human angiotensin converting enzyme 2 (hACE2) transgenic mice, B6.Cg-Tg(K18-ACE2)2Prlmn/J (Stock No: 034860, K18 hACE2) were purchased from The Jackson Laboratory (Bar Harbor, ME).

K18 hACE2 transgenic and WT C57BL/6 mice were either mock (PBS)-infected (controls) or infected intranasally (i.n.) with 1×105 PFU of SARS-CoV-2 in a final volume of 50 μl following isoflurane sedation. After viral infection, mice were monitored daily for morbidity (body weight) and mortality (survival). Mice showing >25% loss of their initial body weight were defined as reaching experimental end-point and humanely killed.

Antibodies were administered 1 day prior to infection using doses of 0.5 mg/mL or 1.5 mg/mL using groups of 10 mice per antibody and results presented as the percentage of 10 animals that survived at 10 days post-infection.

### Reference:

Oladunni FS, Park JG, Pino PA, Gonzalez O, Akhter A, Allud-Guardia A, Olmo-Fontinez A, Gautam S, Garcia-Vilanova A, Ye C, Chiem K, Headley C, Dwivedi V, Parodi LM, Alfson KJ, Staples HM, Schami A, Garcia JI, Whigham A, Platt RN 2nd, Gazi M, Martinez J, Chuba C, Earley S, Rodriguez OH, Mdaki SD, Kavelish KN, Escalona R, Hallam CRA, Christie C, Patterson JL, Anderson TIC, Carrion R Jr, Dick EJ Jr, Hall-Ursone S, Schlesinger LS, Alvarez X, Kaushal D, Giavedoni LD, Turner J, Martinez-Sobrido L, Torrelles JB. Lethality of SARS-CoV-2 infection in K18 human angiotensin-converting enzyme 2 transgenic mice. Nat Commun. 2020 Nov. 30; 11(1):6122. doi: 10.1038/s41467-020-19891-7. PMID: 33257679; PMCID: PMC7705712.

### Example 12: Mouse Immunisations, Sample Collection and Serum Titer

In an additional study, Kymab mice, which have transgenic immunoglobulin loci containing human heavy and light chain gene segments, were immunized with SARS-CoV-2 spike in a variety of immunisation regimens and immunogenic formats, and antigen-specific B cells were selected from the immunised mice. Single B cells from spleen and lymph node samples were sorted using antigen specific probes. Immunogenic formats included (i) nucleic acid encoding full length spike or (ii) nucleic acid encoding full length spike protein with furin cleavage site mutation (GSAS substitution at residue 682-685). Sorting probes used were soluble trimeric spike extracellular domain protein comprising furin cleavage site mutation, double proline mutation and C-terminal T4 fibritin (foldon) trimerisation motif and full length trimeric spike protein presented with furin cleavage site mutation and double proline mutation on green fluorescent protein (GFP) virus like particles (VLPs) generated from host cells.

In a first study, 9,430 antigen specific B-cells were recovered after B cell sorting from immunised mice. From 303 of these B-cells, complete antibody heavy and light chain encoding nucleic acids were recovered and expressed as fully human IgG1 antibodies in mammalian host cells to be taken forward for screening.

In a second study, 10,332 antigen specific B cells were recovered after B cell sorting from immunised mice, and from 299 of these B-cells, complete antibody heavy and light chain encoding nucleic acids were recovered and expressed as fully human IgG1 antibodies in mammalian host cells to be taken forward for screening.

All antibodies taken forward for screening were expressed as fully human IgG1.

### Antigen/Immunogen Preparation

To generate purified proteins for use in sorting of B cells, DNA sequences were generated encoding ECD of trimeric spike protein containing furin cleavage site mutation (GSAS substitution at residue 682-685) and stabilising double proline (PP) mutations (K986 and V987). Coding sequence was fused with N-terminal leader sequence and C-terminal T4 fibritin (foldon) trimerization motif and His tag, codon optimised for mammalian expression and expressed in Expi293F cells. Protein was sequentially purified by a HisTrap HP column. Purified protein was analyzed by binding assays, and then aliquoted and stored at4° C.

Various antigen expressing stable cell lines were generated for different purposes. Antigen DNA sequence was cloned into an expression vector under the control of the CMV promoter flanked by 3′ and 5′ piggyBac specific terminal repeat sequences, which facilitated stable integration into the cell genome. The expression vector contained a puromycin selection cassette to facilitate stable cell line generation. Constructs were transfected into Expi293F cell line or HEK293T cell line, cultured under puromycin selection for 2 weeks and spike expression was validated by using flow cytometry to check for antigen surface expression using monoclonal antibodies. VLPs were generated from Expi293F cells stably co-expressing PP stabilized furin mutated trimeric spike with retroviral Group Antigens (gag) proteins and used for sorting specific B cells.

TABLE E12-1
Stable cell lines and expressing antigen

| Cell line | Expressing antigen |
|---|---|
| 293T - WT spike | Full length spike protein |
| 293T - WT spike, RFP | Full length spike protein, red fluorescence |
| | protein |
| 293T - spike FM, 18d | Full length spike protein with furin cleavage |
| | site mutation and C-terminal retention |
| | sequence 18 amino acid deletion |
| 293T - hACE2, | Full length human ACE2, full length human |
| TMPRSS2 | TMPRSS2 |
| 293T - hACE2, | Full length human ACE2, full length human |
| TMPRSS2, EGFP | TMPRSS2, enhanced green fluorescence |
| | protein |
| Expi293F - spike FM, | Full length spike protein with furin cleavage |
| 2P, 18d | site mutation, double proline mutation and |
| | C-terminal retention sequence 18 amino |
| | acid deletion |
| Expi293F - spike ECD | Extracellular domain of spike protein end at |
| | Q1208 with furin cleavage site mutation and |
| | double proline mutation, followed by T4 |
| | fibritin (foldon) trimerization motif and |
| | 6xHis tag |
| Expi293F - hACE2 ECD | Extracellular domain of human ACE2 end at |
| | S740 and followed by 6xHis tag |

### Example 13: Cell Binding Assay (Primary Screen)

The binding ofthe recovered antibodies (see Example 12) to the spike protein expressing cells was detected by flow cytometric analysis. All antibodies were tested in a single point assay at 5 μg/mL for their binding to the spike protein. Reference mAb A was included as the positive control for the staining in each assay. The binding of antibodies to two version of spike-expressing cell-lines, furin mutated spike (Expi293F-spike FM, 2P, 18d) and WT spike, were tested separately.

The 602 selected antibodies from Example 12 were screened. Among them, 508 were positive binders to furin mutated spike (Expi293F-spike FM, 2P, 18d) and 499 showed positive binding to WT spike. Criteria for passing this cell-based assay was that the geometric mean of the tested antibodies over the threshold set by the average geometric mean of isotype control plus three times of standard deviation.

### Materials and Methods

Supernatants collected from suspension Expi293F cells transfected with expression vectors encoding human IgG1 heavy and light chains were screened for binding to spike protein expressed on the cell surface. Two versions of spike expressing cell-lines, mutated spike and WT spike, were used in this screening. Twenty thousand cells from either cell-line resuspended in FACS buffer (PBS (Corning 21-040-CMR), 1% BSA (Sigma SI-A7906-100G), 2 mM EDTA (J.T. Baker 4040-01)) were plated into each well in 96-well V bottom plates (Greiner 651901) and incubated with 50 μL of collected supernatants normalized to 5 μg/mL or reference mAb A with 10 points of 3-fold dilutions starting from 15 μg/ml on ice for one hour. Before detection by secondary antibody, samples were washed once with FACS buffer to remove extra or non-binding antibodies. The anti-hIgG-AF647 (Jackson ImmunoResearch 109-606-170) was used as the detection antibody at the final concentration of 2 μg/mL. Samples were incubated with detection antibody on ice for additional 30 minutes in the dark followed by washing once with FACS buffer. Cells were then fixed with 2% paraformaldehyde (PBS (Corning 21-040-CMR), 4% paraformaldehyde (Alfa Aesar J61899) for 15 mins at room temperature. Samples were resuspended in 2 mM EDTA PBS buffer (PBS (Coming 21-040-CMR), EDTA (.T.Baker 4040-01) priorto analysis on Cytoflex (Beckman Coulter). The intensity of AF647 fluorochrome was acquired and calculated into the geometric mean of the fluorescence for downstream analysis.

### EXAMPLE 14: ANTIBODY BINDING BY HTRF

HTRF assay was used for primary screening to establish binding of the recovered antibodies (see Example 12) to purified spike proteins. All antibodies were screened in an initial single point assay at 0.5 μg/mL for binding to the RBD domain (Acro Biosystems SPD-C52H3), the NTD subunit (Acro Biosystems SlD-C52H6), and the S2 subunit (Acro Biosystems S2N-C52H5) of SARS-CoV-2 virus.

Criteria for passing the HTRF primary screen were that the antibody bound RBD domain, the NTD subunit, and the S2 subunit of SARS-CoV-2 virus with a ΔF value set out in Table E14-1.

TABLE E14-1
Summary of number of clones meeting primary screening
selection criteria for binding to RBD, NTD, and S2 subunits.

| | Screening batch 1: 303 clones | | Screening batch 2: 299 clones | |
|---|---|---|---|---|
| Assay | Selection criteria | No. hits | Selection criteria | No. hits |
| RBD | Delta F > 11 | 120 | Delta F > 9.5 | 69 |
| NTD | Delta F > 7 | 73 | Delta F > 5 | 93 |
| S2 | Delta F > 30 | 54 | Delta F > 4.2 | 129 |

Table details number of antibodies screened, number of positive hits, and associated cut-off values.

Based on these data, we characterised clones which met the HTRF binding criteria for RBD, NTD, or S2 subunits. Binding data (presented as delta F) for a selection of these antibody clones are shown in Table E14-2.

TABLE E14-2
HTRF binding to SARS-COV-2 spike protein subunits.

| Clone | Binding RBD | Binding NTD | Binding S2 |
|---|---|---|---|
| YANG-1112 | 1790.31 | 2.23 | 6.22 |
| YANG-1301 | 24.45 | 560.30 | 11.65 |
| YANG-1302 | −2.72 | 1255.41 | 28.18 |
| YANG-1401 | 1124.53 | 137.53 | 6.56 |
| YANG-2107 | 1853.07 | 2.40 | −2.19 |
| YANG-2108 | 2124.30 | −5.78 | −7.69 |
| YANG-2111 | 858.86 | −0.02 | −2.48 |
| YANG-2203 | 6.05 | 3.20 | 2247.39 |
| YANG-2204 | −3.24 | −6.44 | 1988.25 |
| YANG-2205 | −0.88 | −2.91 | 1952.91 |
| YANG-2206 | −4.93 | −3.96 | 2117.65 |
| YANG-2207 | 7.00 | 2.37 | 1631.78 |
| YANG-2208 | 5.09 | −0.40 | 2124.64 |
| YANG-2301 | −4.07 | 715.56 | −1.50 |
| YANG-2302 | −3.86 | 913.80 | 6.63 |
| YANG-2303 | 0.38 | 1229.03 | −1.33 |

HTRF delta F values for binding of representative antibody clones to the RBD domain, NTD, and S2 subunits of SARS-COV-2 virus.

### Materials and Methods

Supernatants collected from suspension Expi293 cells transfected with expression vectors encoding human IgG1 heavy and light chains were screened for binding to the RBD domain (Acro Biosystems SPD-C52H3), the NTD subunit (Acro Biosystems S1D-C52H6), and the S2 subunit (Acro Biosystems S2N-C52H5) of SARS-CoV-2 virus. All purchased proteins were in-house labelled with AF647 for HTRF assay. 5 μL/well of supernatants normalised to 0.5 μg/mL were plated into 384 well white HTRF plates (Greiner 784904-012). 5 μL/well of positive control (In-house produced mAb A for RBD binding assay; Leinco Technologies anti-SARS-COV-2 Spike NTD LT2000 for NTD binding assay; IMPI-013 for S2 binding assay) and negative control (non-binding human IgG1, in-house) antibodies were added to the required wells in Expi293 expression medium (Gibco A1435-01) to give the final concentration of lnM. 5 μL/well of RBD diluted at 80 nM, NTD at 80 nM, or S2 at 80 nM in HTRF buffer (PBS (Coming, 21-040-CMR, 0.1% BSA (Sigma SI-A7906), 0.53M KF (Sigma, UR-42216-500G) were added on top on the supernatants to give final concentrations of 20 nM. 10 μL/well of polyclonal Ab anti-human IgG-Eu Cyrptate (Cisbio 61HFCKLB) diluted at 0.25 ug/mL in HTRF buffer were added to wells. Plates were left at room temperature and incubated in the dark for 3 hours for RBD, NTD, and S2 binding assay. Plates were then read on a ClarioStar (BMG Labtech) using 330 nm excitation and detecting emission at 620 and 670 nm.

### Example 15: HTRF RBD:Ace2 Neutralization Assays

An HTRF assay was designed to screen for capacity of the recovered antibodies (see Example 12) to neutralize the binding between the RBD domain of the spike protein of SARS-CoV-2 virus and its ligand the human ACE2 protein. All antibodies were screened as supernatants at a single concentration of 10 μg/mL.

The 602 selected antibodies from Example 12 were screened and 51 clones showed neutralisation activity, based on a criterion of <50% effect in this assay.

### Materials and Methods

Supernatants collected from suspension Expi293F transfected with expression vectors encoding human IgG1 heavy and light chains as above were screened for neutralising the binding between the RBD domain (Acro Biosystems SPD-C52H3) of the spike protein of SARS-CoV-2 virus and its ligand the human ACE2 protein, used here directly labelled with Alexa Fluor 647. 5 μL/well of supernatants normalised to 10 μg/mL were plated into 384 well white HTRF plates (Greiner 784904-012). 5 μL/well of positive (mAB A) and negative control (non-binding human IgG1, in-house) antibodies were added to the required wells in Expi293 expression medium (Gibco A1435-01). Positive and negative control antibodies were added at 40 nM to give a final concentration of 10 nM. 5 μL/well of RBD protein diluted at 40 nM in HTRF buffer (PBS (Coming, 21-040-CMR, 0.1% BSA (Sigma SI-A7906), 0.53M KF (Sigma, UR-42216-500G)) were added on top on the supernatants to give a final concentration of 10 nM. After a 30-min incubation at room temperature, 5 μL/well of AF647-labelled human ACE2 diluted at 20 nM in HTRF buffer to give a final concentration of 5 nM was added to the wells followed 1-hour later, by the addition of 5 μL/well of an anti-Histidine europium cryptate monoclonal antibody (Cisbio 61HISKLB) diluted at 5.3 nM in HTRF buffer to the wells. Plates were left to incubate at room temperature for 2 hours and then read on a ClarioStar (BMG Labtech) using 330 nm excitation and detecting emission at 620 and 670 nm.

Calculation of % of Effect for HTRF neutralisation assay.

$$\% Effect = \frac{( HTRF {Ratio}_{sample} - HTRF {Ratio}_{minimum} )}{( HTRF {Ratio}_{maximum} - HTRF {Ratio}_{minimum} )} \times 100$$

Wells containing 5 nM of AF647 ACE2, 10 nM of spike RBD and 10 nM of human IgG1 isotype control are referred to as maximum signal (HTRF Ratioₘₐₓᵢₘᵤₘ) and wells containing 5 nM of AF647 ACE2, 10 nM of spike RBD and 10 nM of positive control antibody (Sino Biological 40590-D001) as minimum signal (HTRF Ratioₘᵢₙᵢₘᵤₘ).

### Example 16: Pseudovirus Neutralisation Assay

We screened all recovered antibodies (see Example 12) to evaluate their activity in pseudovirus neutralization assay using non-replication-competent pseudoviral particles with SARS-CoV-2 spike protein bearing furin mutation and truncation in retention sequence presented in the pseudoviral envelope.

The 602 selected antibodies from Example 12 were initially tested as two-point titrations (7 nM and 0.7 nM) in the pseudovirus neutralization assay. All tested antibodies were classified into 3 groups, RBD, NTD, and S2 binders, based on the results from HTRF subunit binding assay. In each category, antibodies were ranked by the activity of pseudovirus neutralization assay. The top 10 percent of the antibodies in each category, which in total is 59 antibodies out of 602 recovered antibodies, were selected and taken into a secondary screening phase.

### Materials and Methods

Generation of SARS-CoV-2 recombinant pseudotype virus Non-replicative pseudoparticles were generated using plasmids obtained from RNAi core, Academia Sinica, Taiwan. In brief, 293T stably expressing spike with furin mutation and truncation at the retention sequence, was seeded overnight in a 175T flask for reaching to 80% confluency. The following day, the cells were transfected with 55 g package plasmid (gag-pol), and 55 g pLAS2w.FLuc.Ppuro (a DNA plasmid encoding firefly luciferase as a reporter gene) using the Lipo3000 (Gibco L300-015) according to manufacturer's instructions. After overnight incubation, the culture medium was removed and replenished with DMEM medium containing 1% BSA to enhance the yield of virus. The virus containing supernatant, collected at 24 and 48 hours post transfection, was sterile filtered using 0.45 uM filter. The collected virus was aliquoted and stored at −80° C. for further usage.

Two-point Pseudovirus Neutralization Assay in primary screen

Antibodies purified from supernatant were tested for neutralisation of the SARS-CoV-2 spike pseudovirus in a 96-well format, cell-based viral neutralization assay. All antibodies were initially tested as two-point titrations (7 nM and 0.7 nM of final concentration). Antibody titrations were incubated with SARS-CoV-2 pseudovirus particles encoding firefly luciferase at 37° C. with 5% C02 for 1 hour before adding 50 thousand cells of 293T stable cell-line expressing human recombinant ACE2 and TMPRSS2. TMPRSS2 is a protease which cleaves the spike protein into its S1 and S2 subunits to allow the virus to attach to ACE2 and enter the cell. When the genome of the pseudovirus particles integrates into the host cell genome after entry into cells, firefly luciferase expression is proportional to the number of cells that were transduced. After 48 hrs incubation, the cells are lysed and comparison between the luciferase signals detected in cells only, in cells transduced with pseudotype virus only, and in cells transduced with pseudotype virus in the presence of antibodies, enables determination of neutralization activity against the pseudotype tested.

### Example 17: Epitope Binning

For a competition study, a premix assay with anti-hIgG Fc capture (AHC) biosensor was used and performed on Octet (Fortebio). First, 10 μg/mL of purified spike ECD was mixed with 50 μg/mL of monoclonal antibody (Ab2) at room temperature for 30 minute to form complex. Purified monoclonal antibody (Ab1) at 15 μg/mL was captured by AHC biosensor for 400 s to saturated. AHC sensor was then blocked with 15 μg/mL irrelevant control hIgG antibody for 200 s. Ag-Ab2 complex was added to Ab1 loaded biosensor to measure binding for 200s. Results were analyzed with Data Analysis HT 10.0 software.

The 59 antibodies met the selection criteria described in Example 17 were analysed for competition with reference antibodies. Among them, there were 32 anti-RBD antibodies, 12 anti-NTD antibodies, and 15 anti-S2 antibodies. Reference antibodies used for competition were IMPI-059 and mAb B for RBD, mAb E for NTD, and IMP-013 for S2.

The tested anti-RBD antibodies could be grouped into IMPI-059-like, mAb B-like, cross bin and unique antibodies. The anti-NTD and anti-S2 antibodies did not compete with the reference antibodies mAb E and IMPI-013 respectively.

TABLE E17-1
Anti-RBD antibodies grouped as competing binding
against reference antibodies.

| | IMPI-059- | mAb B- | Cross | Unique |
|---|---|---|---|---|
| | like | like | bin | antibody |
| No. of antibodies | 17 | 3 | 9 | 3 |

### EXAMPLE 18: 11-Point Titration Pseudovirus Neutralization Assay in Secondary Screen

Antibodies were tested in the pseudovirus neutralization assay with 11-point titrations. The purified antibodies were initially diluted 3-fold in DMEM media containing 10% heat-inactivated fetal bovine serum (Gibco) in a 96-well plate. Antibodies were then further titrated into a 96 well plate at 11-point dilution in duplicates starting from 100 nM of final concentration. Positive (mAb A, mAb B, and IMPI-059) control antibodies were also diluted at the same concentrations. 30 μL/well oftitrated antibodies were plated into Isoplate™ 96 well TC plates (PekinElmer 6005071). The following controls were used: cells only, and cells and virus (no antibody). 50 μL/well of pseudotype virus was then added (except to the cells only control). The plates containing the mixture of the antibodies and pseudotype virus were left to incubate for 1 hour at 37° C. with 5% CO₂. 293T cells stably expressing recombinant human ACE2 and TMPRSS2 were then added to each well to obtain a final cell number of 5×10⁴ cells per well. The plates were incubated at 37° C. with 5% CO2. After 48 hours incubation, culture medium was carefully removed and 100 μl of BrightGlo (Promega cat #E2610) was added to each well as the detection reagent. The plate was read on a ClarioStar (BMG Labtech) to detect luminescence intensity. The neutralization curve and IC₅₀ of each antibody was calculated using curve fitting program based on the luciferase activity, normalised to cells only (100%) and virus/cells only (no antibody) as 0%. Neutralisation potency (IC50) for representative clones against different domains is shown in Table E18-1.

TABLE E18-1
Potency of antibodies in pseudovirus neutralisation assay

| Clone | IC50 (nM) | Binding domain |
|---|---|---|
| YANG-1112 | 0.52 | RBD |
| YANG-1301 | 0.52 | NTD |
| YANG-1302 | 0.23 | NTD |
| YANG-1401 | 0.53 | RBD |
| YANG-2107 | 0.3 | RBD |
| YANG-2108 | 0.27 | RBD |
| YANG-2111 | 1.33 | RBD |
| YANG-2203 | 5.45 | S2 |
| YANG-2204 | 3.3 | S2 |
| YANG-2205 | 3.35 | S2 |
| YANG-2206 | 3.96 | S2 |
| YANG-2207 | 2.59 | S2 |
| YANG-2208 | 4.68 | S2 |
| YANG-2301 | 0.53 | NTD |
| YANG-2302 | 0.61 | NTD |
| YANG-2303 | 0.48 | NTD |

Neutralisation potency of selected antibodies against SARS COV2 pseudotype virus expressed as IC50 values in nanomolar.

### Example 19: Syncytia Inhibition Assay

Syncytia formation was observed in SARS-CoV-2 infected cells, including in vivo cell model and histopathologic lung sections. Spike proteins on infected cell surface binding to ACE2 on healthy cells could induce cell fusion. Recently, syncytia formation was reported not only to facilitate virus transmission, but also induce lymphocyte loss (Zhang, Z. et al., Cell Death Differ 28, 2765-2777 (2021)). We measured the ability of selected antibodies to inhibit the syncytia formation by cell-based syncytia formation assay.

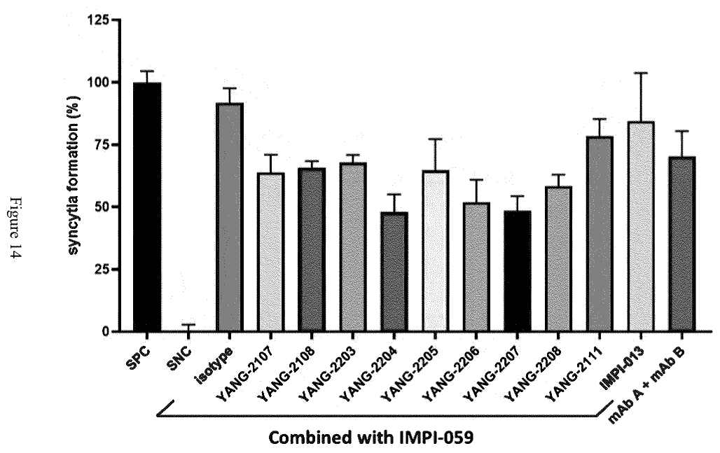

30 selected antibodies were assessed for their ability to inhibit syncytia formation. In combination with the potent anti-RBD antibody IMPI-059, eight antibodies showed comparable or superior inhibition of syncytia formation, as compared to the reference antibody combination mAb A and mAb B (FIG. 14). YANG-2204, YANG-2206, and YANG-2207 were the most potent antibodies in the syncytia inhibition assay.

### Materials and Methods

First, 104 of target cells (293T—hACE2, TMPRSS2, EGFP) were seeded to poly-D-lysine coated 96 well culture plates and incubated for 5 hours. The test antibody and IMPI-059 were diluted with sterile DPBS and mix to 200 nM for each antibody. Effector cells (293T—WT spike, RFP) were diluted to 2.5×10⁴ cells/mL with culture medium. Mixed 40 μL of effector cells and 10 uL of antibody mixture and incubated for 30 minutes on ice, then added to cultured target cells. After 2 days incubation, syncytia formation was checked under inverted fluorescence microscope. Cells were stained by NucBlue™ Live ReadyProbes™ Reagent (Invitrogen, Cat. R37605), fixed with 4% paraformaldehyde and washed with PBS. Plates were covered with aluminum foil and stored in fridge for high content imaging.

Image acquisition and analysis was performed on MetaXpress High content (Molecular Devices). Images of DAPI, GFP and TexasRed channel were acquired under 4× magnification and analyzed. Nucleus was defined by DAPI channel and cell was defined by GFP/TexasRed channel. Definition mask was set by size and intensity to identify nucleus, GFP expressing cell and RFP expressing cells. GFP and RFP colocalized cell was defined as syncytium. Syncytium formation percentage was calculated as nucleus counts in syncytia divided by nucleus counts in whole field.

Conclusions from additional study:

Results of from the study described in Examples 12 to 19 above are summarised in Table E19-1 below.

TABLE E19-1
Results of screening assays from additional antibody generation study

| | | | | | | | syncytia |
|---|---|---|---|---|---|---|---|
| | Affinity | | PV | | PV IC50 | | inhibition |
| | (SPR) by | | neutralization | PV | against | ACE2 | assay |
| mAb | Octet | | % (Ab 1 | neutralisation | mutant | inhibition | combined with |
| name | (nM) | epitope | ug/mL) | IC50 (nM) | (nM) | assay (%) | IMPI-059 (%) |
| YANG-1101 | <0.1 | RBD | 97.99 | | | 99.98 | |
| YANG-1102 | <0.1 | RBD | 86.92 | 0.84 | | 32.51 | |
| YANG-1103 | <0.1 | RBD | 100.05 | 0.28 | | 100.99 | |
| YANG-1105 | <0.1 | RBD | 97.16 | | | 91.44 | |
| YANG-1106 | <0.1 | RBD | 99.48 | | | 100.89 | |
| YANG-1107 | <0.1 | RBD | 99.23 | | | 100.39 | |
| YANG-1108 | <0.1 | RBD | 95.21 | | | 95.91 | |
| YANG-1109 | <0.1 | RBD | 99.44 | 1.81 | | 101.49 | |
| YANG-1110 | <0.1 | RBD | 99.35 | | | 100.76 | |
| YANG-1111 | <0.1 | RBD | 72.69 | 1.82 | | 36.6 | 16.54 |
| YANG-1112 | <0.1 | RBD | 89.22 | 0.52 | 0.34 | 93.41 | |
| YANG-1113 | <0.1 | RBD | 97.61 | | | 96.46 | |
| YANG-1114 | <0.1 | RBD | 93.16 | | | 84.29 | |
| YANG-1115 | <0.1 | RBD | 97.82 | | | 92.03 | |
| YANG-1116 | <0.1 | RBD | 99.24 | | | 100.45 | |
| YANG-1117 | <0.1 | RBD | 97.79 | | | 96.87 | |
| YANG-1118 | <0.1 | RBD | 99.32 | 0.25 | 2.76 | 101.18 | |
| YANG-1119 | <0.1 | RBD | 99.05 | 0.96 | | 90.47 | |
| YANG-1201 | <0.1 | S2 | 40.56 | N/A | | 5.43 | 14.84 |
| YANG-1202 | <0.1 | S2 | 51.93 | N/A | | 3.59 | 32.09 |
| YANG-1203 | <0.1 | S2 | 41.41 | N/A | | 3.27 | 22.04 |
| YANG-1204 | <0.1 | S2 | 44.76 | N/A | | 2.86 | 16.95 |
| YANG-1205 | <0.1 | S2 | 40.18 | N/A | | 1.14 | 20.76 |
| YANG-1206 | <0.1 | S2 | 45.87 | N/A | | 8.71 | 23.69 |
| YANG-1207 | <0.1 | S2 | 39.94 | N/A | | 11.03 | 23.42 |
| YANG-1301 | <0.1 | NTD | 60.44 | 0.52 | | 4.15 | 51.93 |
| YANG-1302 | <0.1 | NTD | 60.27 | 0.23 | 0.25 | −1.28 | 51.83 |
| YANG-1303 | <0.1 | NTD | 46.26 | N/A | | 3.51 | 46.57 |
| YANG-1304 | <0.1 | NTD | 41.46 | N/A | | 16.44 | 39.66 |
| YANG-1305 | <0.1 | NTD | 60.5 | 0.42 | | 6.15 | 41.72 |
| YANG-1401 | <0.1 | RBD | 91.64 | 0.53 | | 3.16 | |
| YANG-1402 | <0.1 | RBD | 94.45 | | | 7.18 | |
| YANG-1403 | <0.1 | RBD | 99.31 | | | 9.18 | |
| YANG-2101 | <0.1 | RBD | 99.96 | | | 100.79 | |
| YANG-2102 | <0.1 | RBD | 99.36 | | | 100.87 | |
| YANG-2103 | <0.1 | RBD | 97.66 | | | 101.96 | |
| YANG-2104 | <0.1 | RBD | 99.34 | | | 101.44 | |
| YANG-2105 | <0.1 | RBD | 99.61 | | | 101.46 | |
| YANG-2106 | <0.1 | RBD | 93.31 | | | 99.53 | |
| YANG-2107 | <0.1 | RBD | 61.2 | 0.3 | | 91.69 | 36.11 |
| YANG-2108 | <0.1 | RBD | 81.84 | 0.27 | 0.23 | 98.27 | 34.17 |
| YANG-2109 | <0.1 | RBD | 94.97 | 0.5 | | 95.13 | 13.43 |
| YANG-2110 | <0.1 | RBD | 99.92 | | | 101.89 | |
| YANG-2111 | <0.1 | RBD | 86.78 | 1.33 | 1 | 101.54 | 21.47 |
| YANG-2112 | <0.1 | RBD | 88.64 | 0.65 | | 29.13 | 29.03 |
| YANG-2201 | <0.1 | S2 | 35.21 | N/A | | −3.21 | −11.3 |
| YANG-2202 | <0.1 | S2 | 37.53 | N/A | | 4.17 | 4.15 |
| YANG-2203 | <0.1 | S2 | 35.69 | 5.45 | 32.46 | 4.38 | 32.15 |
| YANG-2204 | <0.1 | S2 | 40.99 | 3.3 | 8.83 | −2.49 | 52.03 |
| YANG-2205 | <0.1 | S2 | 47.55 | 3.35 | | 5.6 | 35.23 |
| YANG-2206 | <0.1 | S2 | 41.99 | 3.96 | 100 | 0.33 | 48.12 |
| YANG-2207 | <0.1 | S2 | 57.82 | 2.59 | 32.3 | 5.18 | 51.33 |
| YANG-2208 | <0.1 | S2 | 38.16 | 4.68 | | −6.02 | 41.67 |
| YANG-2301 | <0.1 | NTD | 56.43 | 0.53 | 0.4 | 1.45 | 47.9 |
| YANG-2302 | <0.1 | NTD | 52.16 | 0.61 | | −6.05 | 43.26 |
| YANG-2303 | <0.1 | NTD | 58.62 | 0.48 | | 0.27 | 45.9 |
| YANG-2304 | <0.1 | NTD | 52.82 | 0.74 | | 5.58 | 33.77 |
| YANG-2305 | <0.1 | NTD | 64.37 | 0.57 | | 29.71 | 34.78 |
| YANG-2306 | <0.1 | NTD | 60.36 | 0.43 | | 25.07 | 41.18 |

In this additional study to select antibodies against COVID-19 from Kymab mice, as described above in Examples 12 to 19, eleven potent candidate antibodies were identified as a lead panel, including five antibodies against RBD and six antibodies against S2 domain. Two strong anti-RBD antibodies YANG-1112 and YANG-2111 are of interest for not competing with any reference antibodies on binding, indicating the possible unique epitopes recognized by these two antibodies. Additionally, the anti-S2 antibodies YANG-2204, YANG-2206, and YANG-2207 represent the strongest candidates to inhibit the formation of syncytia, i.e., cell fusion, that could be used to pair with an antd-RBD antibody for combination therapy.

### SEQUENCES

### Table 1a

Table 1a below shows amino acid sequences of antibodies and encoding nucleic acids described in this specification. All IMPI VH domains, IMPI VL domains, IMPI CDRs, IMPI heavy chains and IMPI light chains, antibodies comprising them, as well as their encoding nucleic acids, represent examples of the present a invention. CDRs are determined according to IMGT method.

| SEQ | | | |
|---|---|---|---|
| ID | Clone | De- | |
| NO | ID | scription | Sequence |
| 1 | IMPI-052 | VH domain | GAGGTGCAGCTGGTGGAGTCTGGAGGAGGCTTGATCCAGCCTGGGGGGTCCCTGAGACTCTCCTGTGCAGCCTCTGG |
| | | nucleic | GGTCACCGTCAGTAGCAACTACATGAACTGGGTCCGCCAGGCTCCAGGGAAGGGGCTGGAGGGGGTCTCAGTTATTT |
| | | acid | ATAGTGGTGGTACCACATACTACGCAGACTCCGTGAAGGGCCGATTCACCATCTCCAGAGACAATTCCAAGAACACG |
| | | sequence | CTGTATCTTCAAATGAACAGCCTGAGAGCCGAGGACACGGCCGTGTATTACTGTGCGAGAGATACGGTGGTCTACGG |
| | | | TATGGACGTCTGGGGCCAAGGGACCACGGTCACCGTCTCCTCA |
| 2 | IMPI-052 | VH domain | EVQLVESGGGLIQPGGSLRLSCAASGVTVSSNYMNWVRQAPGKGLEGVSVIYSGGTTYYADSVKGRFTISRDNSKNT |
| | | amino acid | LYLQMNSLRAEDTAVYYCARDTVVYGMDVWGQGTTVTVSS |
| | | sequence | |
| 3 | IMPI-052 | HCDR1 | GVTVSSNY |
| 4 | IMPI-052 | HCDR2 | IYSGGTT |
| 5 | IMPI-052 | HCDR3 | ARDTVVYGMDV |
| 6 | IMPI-052 | VL domain | GACATCCAGTTGACCCAGTCTCCATCCTTCCTGTCTGCATCTGTAGGAGACAGAGTCACCATCACTTGCTGGGCCAG |
| | | nucleic | TCAGGGCATTAGCAGTTATTTAGCCTGGTATCAGCAAAAACCAGGGAAAGCCCCTAAGCTCCTGATCTATACTGCAT |
| | | acid | CCACTTTGCAAAGTGGGGTCCCATCAAGGTTCAGCGGCAGTGGATCTGGGACAGAATTCACTCTCACAATCAGCAGC |
| | | sequence | CTGCAGCCTGAAGATTTTGCAACTTATTACTGTCAACAGCTTAATAGTTACCCGCTCACTTTCGGCGGAGGGACCAA |
| | | | GGTGGAGATCAAA |
| 7 | IMPI-052 | VL domain | DIQLTQSPSFLSASVGDRVTITCWASQGISSYLAWYQQKPGKAPKLLIYTASTLQSGVPSRFSGSGSGTEFTLTISS |
| | | amino acid | LQPEDFATYYCQQLNSYPLTFGGGTKVEIK |
| | | sequence | |
| 8 | IMPI-052 | LCDR1 | QGISSY |
| 9 | IMPI-052 | LCDR2 | TAS |
| 10 | IMPI-052 | LCDR3 | QQLNSYPLT |
| 11 | IMPI-047 | VH domain | GAGGTGCAGCTGGTGGAGTCTGGAGGAGGCTTGATCCAGCCTGGGGGGTCCCTGAGACTCTCCTGTGCAGCCTCTGG |
| | | nucleic | GTTCACCGTCAGTAGCAACTACATGAACTGGGTCCGCCAGGCTCCAGGGAAGGGGCTGGAGTGGGTCTCAGTTATTT |
| | | acid | ATAGCGGTGGTAGCACATACTACGCAGACTCCGTGAAGGGCCGATTCACCATCTCCAGAGACAATTCCAAGAACACG |
| | | sequence | CTGTATCTTCAAATGAACAGCCTGAGAACCGAGGACACGGCCGTGTATTACTGTGCGCGAGATCTAGTGGCTTACGG |
| | | | TATGGACGTCTGGGGCCAAGGGACCACGGTCACCGTCTCCTCA |
| 12 | IMPI-047 | VH domain | EVQLVESGGGLIQPGGSLRLSCAASGFTVSSNYMNWVRQAPGKGLEWVSVIYSGGSTYYADSVKGRFTISRDNSKNT |
| | | amino acid | LYLQMNSLRTEDTAVYYCARDLVAYGMDVWGQGTTVTVSS |
| | | sequence | |
| 13 | IMPI-047 | HCDR1 | GFTVSSNY |
| 14 | IMPI-047 | HCDR2 | IYSGGST |
| 15 | IMPI-047 | HCDR3 | ARDLVAYGMDV |
| 16 | IMPI-047 | VL domain | GACATCCAGTTGACCCAGTCTCCATCCTTCCTGTCTGCATCTGTAGGAGACAGAGTCACCATCACTTGCTGGGCCAG |
| | | nucleic | TCAGGGCATTAGCAGTTATTTAGCCTGGTATCAGCAAAAACCAGGGAAAGCCCCTAAGGTCCTGATCTATGCTGCAT |
| | | acid | CCACTTTGCAAAGTGGGGTCCCATCAAGGTTCAGCGGCAGTGGATCTGGGACAGAATTCACTCTCACAATCAGCAGC |
| | | sequence | CTGCAGCCTGAAGATTTTGCAACTTATTACTGTCAACTACTTAATAGTAACCCGCTCACTTTCGGCGGAGGGACCAA |
| | | | GGTGGAGATCAAA |
| 17 | IMPI-047 | VL domain | DIQLTQSPSFLSASVGDRVTITCWASQGISSYLAWYQQKPGKAPKVLIYAASTLQSGVPSRFSGSGSGTEFTLTISS |
| | | amino acid | LQPEDFATYYCQLLNSNPLTFGGGTKVEIK |
| | | sequence | |
| 8 | IMPI-047 | LCDR1 | QGISSY |
| 18 | IMPI-047 | LCDR2 | AAS |
| 19 | IMPI-047 | LCDR3 | QLLNSNPLT |
| 20 | IMPI-003 | VH domain | GAAGTGCAGCTGGTGGAGTCTGGGGGAGGCTTGGTACAGCCTGGCAGGTCCCTGAGACTCTCCTGTGCAGCCTCTGG |
| | | nucleic | ATTCACCTTTGATGATTATGCCATGCACTGGGTCCGGCAAGCTCCAGGGAAGGGCCTGGAGTGGGTCTCAGGTATTA |
| | | acid | GTTGGAATAGTGGTAGCATAGGCTATGCGGACTCTGTGAAGGGCCGATTCACCATCTCCAGAGACAACGCCAAGAAC |
| | | sequence | TCCCTGTATCTGCAAATGAACAGTCTGAGAGCTGAAGACACGGCCTTATATTACTGTGCAAAAGATTTGGGACTGGG |
| | | | GATTGGCTTCTATTACGGTTTGGACGTCTGGGGCCAGGGGACCACGGTCACCGTCTCCTCA |
| 21 | IMPI-003 | VH domain | EVQLVESGGGLVQPGRSLRLSCAASGFTFDDYAMHWVRQAPGKGLEWVSGISWNSGSIGYADSVKGRFTISRDNAKN |
| | | amino acid | SLYLQMNSLRAEDTALYYCAKDLGLGIGFYYGLDVWGQGTTVTVSS |
| | | sequence | |
| 22 | IMPI-003 | HCDR1 | GFTFDDYA |
| 23 | IMPI-003 | HCDR2 | ISWNSGSI |
| 24 | IMPI-003 | HCDR3 | AKDLGLGIGFYYGLDV |
| 25 | IMPI-003 | VL domain | GCCATCCAAATGACCCAGTCTCCATCCTCCCTGTCTGCATCTGTAGGAGACAGAGTCACCATCACTTGCCGGGCAAG |
| | | nucleic | TCAGGGCATTAGAAATGATTTAGGCTGGTATCAGCAGAAGCCAGGGAAAGCCCCTAAGCTCCTGATCTATGATGCAT |
| | | acid | CCACTTTACAAAGTGGGGTCCCATCGAGGTTCAGCGGCAGTGGATCTGGCACAGATTTCACTCTCACCCTCAGCAGC |
| | | sequence | CTGGAGCCTGAAGATTTAGCAACTTATTACTGTCTACATCACTACACTTACCCGTGGACGTTCGGCCATGGGACCAA |
| | | | GGTGGAACTCAAA |
| 26 | IMPI-003 | VL domain | AIQMTQSPSSLSASVGDRVTITCRASQGIRNDLGWYQQKPGKAPKLLIYDASTLQSGVPSRFSGSGSGTDFTLTLSS |
| | | amino acid | LEPEDLATYYCLHHYTYPWTFGHGTKVELK |
| | | sequence | |
| 27 | IMPI-003 | LCDR1 | QGIRND |
| 28 | IMPI-003 | LCDR2 | DAS |
| 29 | IMPI-003 | LCDR3 | LHHYTYPWT |
| 30 | IMPI-043 | VH domain | GAGGTGCAGCTGGTGCAGTCTGGAGCAGAGGTGAAAAAGCCCGGGGAGTCTCTGAAGATCTCCTGTAAGGGTTCTGG |
| | | nucleic | ATACAGCTTTACCAGCTACTGGATCGGCTGGGTGCGCCAGATGCCCGGGAAAGGCCTGGAGTGGATGGGGATCATCT |
| | | acid | ATCCTGGTGACTCTGATACCAGGAACAGCCCGTCCTTCCAAGGCCAGGTCACCATCTCAGCCGACAAGTCCATCAGC |
| | | sequence | ACCGCCTACCTGCAGTGGAGCAGCCTGAAGGCCTCGGACACCGCCATGTATTACTGTGCGAGGTCGTATAACTGGAA |
| | | | CTACTTTGACTACTGGGGCCAGGGAACCCTGGTCACCGTCTCCTCA |
| 31 | IMPI-043 | VH domain | EVQLVQSGAEVKKPGESLKISCKGSGYSFTSYWIGWVRQMPGKGLEWMGIIYPGDSDTRNSPSFQGQVTISADKSIS |
| | | amino acid | TAYLQWSSLKASDTAMYYCARSYNWNYFDYWGQGTLVTVSS |
| | | sequence | |
| 32 | IMPI-043 | HCDR1 | GYSFTSYW |
| 33 | IMPI-043 | HCDR2 | IYPGDSDT |
| 34 | IMPI-043 | HCDR3 | ARSYNWNYFDY |
| 35 | IMPI-043 | VL domain | GCCATCCAGTTGACCCAGTCTCCATCCTCCCTGTCTGCATCTGTAGGAGACAGAGTCACCATCACTTGCCGGGCAAG |
| | | nucleic | TCAGGGCATTAGCAGTGCTTTAGCCTGGTATCAGCAGAAACCAGGGAAAGCTCCTAAGCTCCTGATCTATGATGCCT |
| | | acid | CCAGTTTGGAAAGTGGGGTCCCATCAAGGTTCAGCGGCAGTGGATCTGGGACAGATTTCACTCTCACCATCAGCAGC |
| | | sequence | CTGCAGCCTGAAGATTTTGCAGCTTATTACTGTCAACAGTTTATTAGTTATCCTTGGACGTTCGGCCAAGGGACCAA |
| | | | GGTGGAAATCAAA |
| 36 | IMPI-043 | VL domain | AIQLTQSPSSLSASVGDRVTITCRASQGISSALAWYQQKPGKAPKLLIYDASSLESGVPSRFSGSGSGTDFTLTISS |
| | | amino acid | LQPEDFAAYYCQQFISYPWTFGQGTKVEIK |
| | | sequence | |
| 37 | IMPI-043 | LCDR1 | QGISSA |
| 28 | IMPI-043 | LCDR2 | DAS |
| 38 | IMPI-043 | LCDR3 | QQFISYPWT |
| 39 | IMPI-048 | VH domain | CAGGTGCAGCTGCAGGAGTCGGGCCCAGGACTGGTGAAGCCTTCACAGACCCTGTCCCTCACCTGCACTGTCTCTGG |
| | | nucleic | TGGCTCCATCAGCAGTTATGGTTACTACTGGAGCTGGATCCGCCAGCACCCAGGGAAGGGCCTGGAGTGGATTGGGT |
| | | acid | ACATCTATTACAGTGGGAGCACCTACTACAACCCGTCCCTCAAGAGTCGAGCTACCATATCAGTAGACACGTCTAAG |
| | | sequence | AACCAGCTCTCCCTGAGGCTGAACTCTGTCAGTGCCGCGGACACGGCCGTGTATTACTGTGCGAGAGACTTCGGTGG |
| | | | TAACTCGAACTACTTTGACTACTGGGGCCAGGGAAGCCTGGTCACCGTCTCCTCA |
| 40 | IMPI-048 | VH domain | QVQLQESGPGLVKPSQTLSLTCTVSGGSISSYGYYWSWIRQHPGKGLEWIGYIYYSGSTYYNPSLKSRATISVDTSK |
| | | amino acid | NQLSLRLNSVSAADTAVYYCARDFGGNSNYFDYWGQGSLVTVSS |
| | | sequence | |
| 41 | IMPI-048 | HCDR1 | GGSISSYGYY |
| 42 | IMPI-048 | HCDR2 | IYYSGST |
| 43 | IMPI-048 | HCDR3 | ARDFGGNSNYFDY |
| 44 | IMPI-048 | VL domain | GCCATCCAGTTGACCCAGTCTCCATCCTCCCTGTCTGCATCTGTAGGAGACAGAGTCTCCATCACTTGCCGGGCAAG |
| | | nucleic | TCAGGTCATTAGTAGTGCTTTAGCCTGGTATCAGCAGAAACCAGGGAAAGCTCCTAAGCTCCTGATCTATGATGCCT |
| | | acid | CCAGTTTGGAAAGTGGGGTCCCATCAAGGTTCAGCGGCAGTGGATCTGGGACAGATTTCACTCTCACCATCAGCAGC |
| | | sequence | CTGCAGCCTGAAGATTTTACAACTTATTACTGTCAACAGTTTAATAGTTACCCTCTCACTTTCGGCGGAGGGACCAC |
| | | | GGTGGAGATCAAA |
| 45 | IMPI-048 | VL domain | AIQLTQSPSSLSASVGDRVSITCRASQVISSALAWYQQKPGKAPKLLIYDASSLESGVPSRFSGSGSGTDFTLTISS |
| | | amino acid | LQPEDFTTYYCQQFNSYPLTFGGGTTVEIK |
| | | sequence | |
| 46 | IMPI-048 | LCDR1 | QVISSA |
| 28 | IMPI-048 | LCDR2 | DAS |
| 47 | IMPI-048 | LCDR3 | QQFNSYPLT |
| 48 | IMPI-014 | VH domain | CAGGTGCAGCTGCAGGAGTCGGGCCCAGGACTGGTGAAGCCTTCACAGACCCTGTCCCTCACCTGCACTGTCTCTGG |
| | | nucleic | TGGCTCCATCAGCAGTTATGGTTACTACTGGAGCTGGATCCGCCAGCACCCAGGGAAGGGCCTGGAGTGGATTGGGT |
| | | acid | ACATCTATTACAGTGGGAGCACCTACTACAACCCGTCCCTCAAGAGTCGAGCTACCATATCAGTAGACACGTCTAAG |
| | | sequence | AACCAGCTCTCCCTGAGGCTGAACTCTGTCAGTGCCGCGGACACGGCCGTGTATTACTGTGCGAGAGACTTCGGTGG |
| | | | TAACTCGAACTACTTTGACTACTGGGGCCAGGGAACCCTGGTCACCGTCTCCTCA |
| 49 | IMPI-014 | VH domain | QVQLQESGPGLVKPSQTLSLTCTVSGGSISSYGYYWSWIRQHPGKGLEWIGYIYYSGSTYYNPSLKSRATISVDTSK |
| | | amino acid | NQLSLRLNSVSAADTAVYYCARDFGGNSNYFDYWGQGTLVTVSS |
| | | sequence | |
| 41 | IMPI-014 | HCDR1 | GGSISSYGYY |
| 42 | IMPI-014 | HCDR2 | IYYSGST |
| 43 | IMPI-014 | HCDR3 | ARDFGGNSNYFDY |
| 50 | IMPI-014 | VL domain | GCCATCCAGTTGACCCAGTCTCCATCCTCCCTGTCTGCATCTGTAGGAGACAGGGTCACCATCACTTGCCGGGCAAG |
| | | nucleic | TCAGGGCATTAGCAGTGCTTTAGCCTGGTATCAGCAGAAACCAGGTAAAGCTCCTAAGCTCCTGATCTATGATGCCT |
| | | acid | CCAGTTTGGAAAGTGGGGTCCCATCAAGGTTCAGCGGCAGTGGATCTGGGACAGATTTCACTCTCACCATCAGCAGC |
| | | sequence | CTGCAGCCTGAAGATTTTACAACTTATTACTGTCAACAGTTTAATAGTTACCCTCTCACTTTCGGCGGAGGGACCAA |
| | | | GGTGGAGATCAAA |
| 51 | IMPI-014 | VL domain | AIQLTQSPSSLSASVGDRVTITCRASQGISSALAWYQQKPGKAPKLLIYDASSLESGVPSRFSGSGSGTDFTLTISS |
| | | amino acid | LQPEDFTTYYCQQFNSYPLTFGGGTKVEIK |
| | | sequence | |
| 37 | IMPI-014 | LCDR1 | QGISSA |
| 28 | IMPI-014 | LCDR2 | DAS |
| 47 | IMPI-014 | LCDR3 | QQFNSYPLT |
| 52 | IMPI-059 | VH domain | GAGGTGCAGCTGGTGGAGTCTGGAGGAGGCTTGATCCAGCCGGGGGGGTCCCTGAGACTCTCCTGTGCAGCCTCTGG |
| | | nucleic | GTTCACCGTCAGTAGCATCTACATGAGCTGGGTCCGCCAGGCTCCAGGGAAGGGGCTGGAGTGGGTCTCAATTATTT |
| | | acid | ATAGCCGTGGTAGTACAGACTACGCAGACTCCGTGAAGGGCCGATTCACCATCTCCAGAGACATTTCCAAGAACACG |
| | | sequence | CTGTATCTTCAAATGAACAGCCTGAGAGCCGAGGACACGGCCGTGTATTACTGTGCGAGAGATGGGACTATGGTTCG |
| | | | GGGAGTTCATGCTTTTGATATCTGGGGCCAAGGGACAATGGTCACCGTCTCTTCA |
| 53 | IMPI-059 | VH domain | EVQLVESGGGLIQPGGSLRLSCAASGFTVSSIYMSWVRQAPGKGLEWVSIIYSRGSTDYADSVKGRFTISRDISKNT |
| | | amino acid | LYLQMNSLRAEDTAVYYCARDGTMVRGVHAFDIWGQGTMVTVSS |
| | | sequence | |
| 54 | IMPI-059 | HCDR1 | GFTVSSIY |
| 55 | IMPI-059 | HCDR2 | IYSRGST |
| 56 | IMPI-059 | HCDR3 | ARDGTMVRGVHAFDI |
| 57 | IMPI-059 | VL domain | GACATCCAGATGACCCAGTCTCCACCCTCCCTGTCTGCATCTATAGGAGACAGAGTCACCATCACTTGCCAGGCGAG |
| | | nucleic | TCAGGACATTAGCAACTATTTAAATTGGTATCAGCAGAAACCAGGGAAAGCCCCTAAGCTCCTGATCTACGATGCAT |
| | | acid | CCAATTTGGAAACAGGGGTCCCATCAAGGTTCAGTGGAAGTGGATCTGGGACAGATTTTACTCTCACCATCAGCAAC |
| | | sequence | CTGCAGCCTGAAGATATTGTAACATATTACTGTCAACAGTATGATAATCTCCCGAGCAGTTTTGGCCAGGGGACCAA |
| | | | GCTGGAGATCAAA |
| 58 | IMPI-059 | VL domain | DIQMTQSPPSLSASIGDRVTITCQASQDISNYLNWYQQKPGKAPKLLIYDASNLETGVPSRFSGSGSGTDFTLTISN |
| | | amino acid | LQPEDIVTYYCQQYDNLPSSFGQGTKLEIK |
| | | sequence | |
| 59 | IMPI-059 | LCDR1 | QDISNY |
| 28 | IMPI-059 | LCDR2 | DAS |
| 60 | IMPI-059 | LCDR3 | QQYDNLPSS |
| 61 | IMPI-057 | VH domain | GAAGTGCAGCTGGTGGAGTCTGGGGGAGGCTTGGTACAGCCTGGCAGGTCCCTGAGACTCTCCTGTGCAGCCTCTGG |
| | | nucleic | ATTCACCTTTGATGATTATGCCATACACTGGGTCCGGCAAGCTCCAGGGAAGGGCCTGGAATGGGTCTCAGGTATTA |
| | | acid | CTTGGAATAGTGGTAGCATAGGCTATGCGGACTCTGTGAAGGGCCGATTCACCATCTCCAGAGACAACGCCAAGAAC |
| | | sequence | TCCCTGTATCTGCAAATGAACAGTCTGAGAGCTGAGGATACGGCCTTGTATTACTGTGCAAAAGATCTCCAGGGGGA |
| | | | CTACTACTACTACGGTATGGACGTCTGGGGCCAAGGGACCACGGTCACCGTCTCCTCA |
| 62 | IMPI-057 | VH domain | EVQLVESGGGLVQPGRSLRLSCAASGFTFDDYAIHWVRQAPGKGLEWVSGITWNSGSIGYADSVKGRFTISRDNAKN |
| | | amino acid | SLYLQMNSLRAEDTALYYCAKDLQGDYYYYGMDVWGQGTTVTVSS |
| | | sequence | |
| 22 | IMPI-057 | HCDR1 | GFTFDDYA |
| 63 | IMPI-057 | HCDR2 | ITWNSGSI |
| 64 | IMPI-057 | HCDR3 | AKDLQGDYYYYGMDV |
| 65 | IMPI-057 | VL domain | GCCATCCAGTTGACCCAGTCTCCATCCTCCCTGTCTGCATCTGTAGGAGACAGAGTCACCATCACTTGCCGGGCTAG |
| | | nucleic | TCAGGACATTAGCAGTGCTTTAGCCTGGTATCAGCAGAAAGCAGGGAAAGCTCCTAAGCTCCTGATCTATGATGGCT |
| | | acid | CCAGTTTTAAAAGTGGGGTCCCATCAAGGTTCAGCGGCAGTAGATCTGGGACAGATTTCACTCTCACCATCAGCAGC |
| | | sequence | CTGCAGCCTGAAGATTTTGCAACTTATTACTGTCAACAGTTTATTAGTTACCCGCTCACTTTCGGCGGAGGGACCAG |
| | | | GGTGGAGATCAAA |
| 66 | IMPI-057 | VL domain | AIQLTQSPSSLSASVGDRVTITCRASQDISSALAWYQQKAGKAPKLLIYDGSSFKSGVPSRFSGSRSGTDFTLTISS |
| | | amino acid | LQPEDFATYYCQQFISYPLTFGGGTRVEIK |
| | | sequence | |
| 67 | IMPI-057 | LCDR1 | QDISSA |
| 68 | IMPI-057 | LCDR2 | DGS |
| 69 | IMPI-057 | LCDR3 | QQFISYPLT |
| 70 | IMPI-015 | VH domain | GAGGTGCAGCTGGTGCAGTCTGGAGCAGAGGTGAAAAAGCCCGGGGAGTCTCTGAAGATCTCCTGTAAGGGTTCTGG |
| | | nucleic | ATACAGCTTTACCATCTACTGGATCGGCTGGGTGCGCCAGATGCCCGGGAAAGGCCTGGAGTGGATGGGGATCATCT |
| | | acid | ATCCTGGTGACTCTGATACCAGATACAGCCCGTCCTTCCAAGGCCAGGTCACCATCTCAGCCGACAAGTCCATCAGC |
| | | sequence | ACCGCCTACCTGCAGTGGAGCAGCCTGAAGGCCTCGGACACCGCCATGTATTACTGTGCGAGGTCGTATAACTGGAA |
| | | | CTACTTTGACTACTGGGGCCAGGGAACCCTGGTCACCGTCTCCTCA |
| 71 | IMPI-015 | VH domain | EVQLVQSGAEVKKPGESLKISCKGSGYSFTIYWIGWVRQMPGKGLEWMGIIYPGDSDTRYSPSFQGQVTISADKSIS |
| | | amino acid | TAYLQWSSLKASDTAMYYCARSYNWNYFDYWGQGTLVTVSS |
| | | sequence | |
| 72 | IMPI-015 | HCDR1 | GYSFTIYW |
| 33 | IMPI-015 | HCDR2 | IYPGDSDT |
| 34 | IMPI-015 | HCDR3 | ARSYNWNYFDY |
| 73 | IMPI-015 | VL domain | GCCATCCAGTTGACCCAGTCTCCATCCTCCCTGTCTGCATCTGTAGGAGACAGAGTCACCATCACTTGCCGGGCAAG |
| | | nucleic | TCAGGGCATTAACAGTGCTTTAGCCTGGTATCAGCAGAAACCAGGGAAAACTCCTAAACTCCTAATCTATGATGCCT |
| | | acid | CCAGTTTGGAAAGTGGGGTCCCATCAAGGTTCAGCGGCAGTGGATCTGGGACAGATTTCACTCTCACCATCAGCAGC |
| | | sequence | CTGCAGCCTGAAGATTTTGCAACTTATTACTGTCAACAGTTTAATAGTTACCCTTGGACGTTCGGCCAAGGGACCAA |
| | | | GGTGGAAATCAAA |
| 74 | IMPI-015 | VL domain | AIQLTQSPSSLSASVGDRVTITCRASQGINSALAWYQQKPGKTPKLLIYDASSLESGVPSRFSGSGSGTDFTLTISS |
| | | amino acid | LQPEDFATYYCQQFNSYPWTFGQGTKVEIK |
| | | sequence | |
| 75 | IMPI-015 | LCDR1 | QGINSA |
| 28 | IMPI-015 | LCDR2 | DAS |
| 76 | IMPI-015 | LCDR3 | QQFNSYPWT |
| 77 | IMPI-025 | VH domain | CAGGTGCAGCTGCAGGAGTCGGGCCCAGGACTGGTGAAGCCTTCGGGGACCCTGTCCCTCACCTGCGCTGTCTCTGG |
| | | nucleic | TGGCTCCATCAGCAGGAGTACCTGGTGGAGTTGGGTCCGCCAGCCCCCAGGGAAGGGGCTGGAGTGGATTGGGGAAA |
| | | acid | TCTCTCATAGTGGGAGCACCCAGTACAACCCGTCCCTCAAGAGTCGAGTCACCATATCACTAGACAAGTCCAAGAAC |
| | | sequence | CAGTTCTCCCTGAAGCTGAACTCTGTGACCGCCGCGGACACGGCCGTATATTACTGTGCGGGAGAGGGGTATAACTG |
| | | | GAACTACTGGGGCCAGGGAACCCTGGTCACCGTCTCCTCA |
| 78 | IMPI-025 | VH domain | QVQLQESGPGLVKPSGTLSLTCAVSGGSISRSTWWSWVRQPPGKGLEWIGEISHSGSTQYNPSLKSRVTISLDKSKN |
| | | amino acid | QFSLKLNSVTAADTAVYYCAGEGYNWNYWGQGTLVTVSS |
| | | sequence | |
| 79 | IMPI-025 | HCDR1 | GGSISRSTW |
| 80 | IMPI-025 | HCDR2 | ISHSGST |
| 81 | IMPI-025 | HCDR3 | AGEGYNWNY |
| 82 | IMPI-025 | VL domain | GATGTTGTAATGACTCAGTCTCCACTCTCCCTGCCCGTCACCCTTGGACAGCCGGCCTCCATCTCCTGCAGGTCTAG |
| | | nucleic | TCAAAGCCTCGTATACAGTGATGGAAACACCTACTTGAGTTGGTTTCAGCAGAGGCCAGGCCAATCTCCAAGGCGCC |
| | | acid | TAATTTATAAGGTTTCTAAGTGGGACTCTGGGGTCCCAGACAGATTCAGCGGCAGTGGGTCAGGCACTGATTTCACA |
| | | sequence | CTGAAAATCAGCAGGGTGGAGGCTGAGGATGTTGGGATTTATTACTGCATGCAAGGTACACACTGGCCGCTCACTTT |
| | | | CGGCGGAGGGACCAAGGTGGAGATCAAA |
| 83 | IMPI-025 | VL domain | DVVMTQSPLSLPVTLGQPASISCRSSQSLVYSDGNTYLSWFQQRPGQSPRRLIYKVSKWDSGVPDRFSGSGSGTDFT |
| | | amino acid | LKISRVEAEDVGIYYCMQGTHWPLTFGGGTKVEIK |
| | | sequence | |
| 84 | IMPI-025 | LCDR1 | QSLVYSDGNTY |
| 85 | IMPI-025 | LCDR2 | KVS |
| 86 | IMPI-025 | LCDR3 | MQGTHWPLT |
| 87 | IMPI-051 | VH domain | GAGGTGCAGCTGGTGCAGTCTGGAGCAGAGGTGAAAAAGCCCGGGGAGTCTCTGAAGATCTCCTGTAAGGGTTCTGA |
| | | nucleic | ATACAGATTTACCAGCTACTGGATCGGCTGGGTGCGCCAGATGCCCGGGAAAGGCCTGGAGTGGATGGGGATCATCT |
| | | acid | ATCCTGGTGACTCTGATACCAGATACAGCCCGTCCTTCCAAGGCCAGGTCACCATCTCAGCCGACAAGTCCATCAGC |
| | | sequence | ACCGCCTACCTGCAGTGGAGCAGCCTGAAGGCCTCGGACACCGCCATGTATTACTGTGCGAGGTCGTATAACTGGAA |
| | | | CTACTTTGACTACTGGGGCCAGGGAACCCTGGTCACCGTCTCCTCA |
| 88 | IMPI-051 | VH domain | EVQLVQSGAEVKKPGESLKISCKGSEYRFTSYWIGWVRQMPGKGLEWMGIIYPGDSDTRYSPSFQGQVTISADKSIS |
| | | amino acid | TAYLQWSSLKASDTAMYYCARSYNWNYFDYWGQGTLVTVSS |
| | | sequence | |
| 89 | IMPI-051 | HCDR1 | EYRFTSYW |
| 33 | IMPI-051 | HCDR2 | IYPGDSDT |
| 34 | IMPI-051 | HCDR3 | ARSYNWNYFDY |
| 90 | IMPI-051 | VL domain | GCCATCCAGTTGACCCAGTCTCCTTCCTCCCTGTCTGCATCTGTAGGAGACAGAGTCACCATCACTTGCCGGCCAAG |
| | | nucleic | TCAGGGCATTAGCAGTGGTTTAGCCTGGTATCAGCAGAAACCAGGGAAAGCTCCTAACCTCCTGATCTATGATGCCT |
| | | acid | CCAGTTTGGAAAGTGGGGTCCCATCAAGGTTCAGCGGCAGTGGATCTGGGACAGATTTCACTCTCACCATCAGCAGC |
| | | sequence | CTGCAGCCTGAGGATTTTGCAACTTATTACTGTCAACAGTTTAAAAGTTACCCTTGGACGTTCGGCCAAGGGACCAA |
| | | | GGTGGAAATCAAA |
| 91 | IMPI-051 | VL domain | AIQLTQSPSSLSASVGDRVTITCRPSQGISSGLAWYQQKPGKAPNLLIYDASSLESGVPSRFSGSGSGTDFTLTISS |
| | | amino acid | LQPEDFATYYCQQFKSYPWTFGQGTKVEIK |
| | | sequence | |
| 92 | IMPI-051 | LCDR1 | QGISSG |
| 28 | IMPI-051 | LCDR2 | DAS |
| 93 | IMPI-051 | LCDR3 | QQFKSYPWT |
| 94 | IMPI-031 | VH domain | GAAGTGCAGCTGGTGGAGTCTGGGGGAGGCTTGGTACAGCCTGGCAGGTCCCTGAGACTCTCCTGTGCAGCCTCTGG |
| | | nucleic | ATTCACCTTTGATGATTATGCCATGCACTGGGTCCGGCAATCTCCAGGGAAGGGCCTGGAGTGGGTCTCAGGTATTA |
| | | acid | GTTGGAATAGTGGTTCCATAGGCTATGCGGACTCTGTGAAGGGCCGATTCACCATCTCCAGAGACAACGCCAAGAAC |
| | | sequence | TCCCTGTATCTGCAAATGAACAGTCTGAGAGCTGAGGACACGGCCTTGTATTACTGTGCAAAAGATCTCCAGGGGGA |
| | | | CGACTACTACTACGGTATGGACGTCTGGGGCCAAGGGACCACGGTCACCGTCTCCTCA |
| 95 | IMPI-031 | VH domain | EVQLVESGGGLVQPGRSLRLSCAASGFTFDDYAMHWVRQSPGKGLEWVSGISWNSGSIGYADSVKGRFTISRDNAKN |
| | | amino acid | SLYLQMNSLRAEDTALYYCAKDLQGDDYYYGMDVWGQGTTVTVSS |
| | | sequence | |
| 22 | IMPI-031 | HCDR1 | GFTFDDYA |
| 23 | IMPI-031 | HCDR2 | ISWNSGSI |
| 96 | IMPI-031 | HCDR3 | AKDLQGDDYYYGMDV |
| 97 | IMPI-031 | VL domain | GCCATCCAGTTGACCCAGTCTCCATCCTCCCTGTCTGCATCTGTAGGAGACAGAGTCACCATCACTTGCCGGGCAAG |
| | | nucleic | TCAGGGCATTAGCAGTGCTTTAGCCTGGTATCAGCAGAAACCAGGGAAAGCTCCTAAGCTCCTGATCTTTGATGCCT |
| | | acid | CCAGTTTGGAAAGTGGGGTCCCATCAAGGTTCAGCGGCAGTGGATCTGGGACAGATTTCACTTTCACCATCAGCAGC |
| | | sequence | CTGCAGCCTGAAGATTTTGCAGCTTTTTACTGTCAACAGTTTATTAGTTACCCGCTCACTTTCGGCGGAGGGACCAA |
| | | | GGTGGAGATCAAA |
| 98 | IMPI-031 | VL domain | AIQLTQSPSSLSASVGDRVTITCRASQGISSALAWYQQKPGKAPKLLIFDASSLESGVPSRFSGSGSGTDFTFTISS |
| | | amino acid | LQPEDFAAFYCQQFISYPLTFGGGTKVEIK |
| | | sequence | |
| 37 | IMPI-031 | LCDR1 | QGISSA |
| 28 | IMPI-031 | LCDR2 | DAS |
| 69 | IMPI-031 | LCDR3 | QQFISYPLT |
| 99 | IMPI-045 | VH domain | CAGGTGCAGCTGCAGGAGTCGGGCCCAGGACTGGTGAAGCCTTCACAGACCCTGTCCCTCACCTGCACTGTCTCTGG |
| | | nucleic | TGGCTCCATCAGCAGTTATGGTTTCTACTGGAGCTGGATCCGCCAGCACCCAGGGAAGGGCCTGGAGTGGATTGGGT |
| | | acid | TCATCTATTACAGTGGGAGCACCTACTACAACCCGTCCCTCAAGAGTCGAGCTACCATATCAGTAGACACGTCTAAG |
| | | sequence | AACCAGCTCTCCCTGAGGCTGAACTCTGTCAGTGCCGCGGACACGGCCGTGTATTACTGTGCGAGAGACTTCGGTGG |
| | | | TAACTCGAACTACTTTGACTACTGGGGCCAGGGAACCCTGGTCACCGTCTCCTCA |
| 100 | IMPI-045 | VH domain | QVQLQESGPGLVKPSQTLSLTCTVSGGSISSYGFYWSWIRQHPGKGLEWIGFIYYSGSTYYNPSLKSRATISVDTSK |
| | | amino acid | NQLSLRLNSVSAADTAVYYCARDFGGNSNYFDYWGQGTLVTVSS |
| | | sequence | |
| 101 | IMPI-045 | HCDR1 | GGSISSYGFY |
| 42 | IMPI-045 | HCDR2 | IYYSGST |
| 43 | IMPI-045 | HCDR3 | ARDFGGNSNYFDY |
| 102 | IMPI-045 | VL domain | GCCATCCAATTGACCCAGTCTCCATCCTCCCTGTCTGCGTCTGTTGGAGACAGAGTCACCATCACTTGCCGGGCAAG |
| | | nucleic | TCAGGGCATTAGCAGTGCTTTAGCCTGGTATCAGCAGAAACCAGGGAAAGCTCCTCAGCTCCTGATCTATGATGCCT |
| | | acid | CCAGTTTGGAAAGTGGGGTCCCATCGAGGTTCAGCGGCAGTGGATCTGGGACAGATTTCACTCTCACCATCAGCAGC |
| | | sequence | CTGCAGCCTGAAGATTTTGCAACTTATTACTGTCAACAGTTTAATAGTTACCCTCTCACTTTCGGCGGAGGGACCAA |
| | | | GGTGGAGATCAAA |
| 103 | IMPI-045 | VL domain | AIQLTQSPSSLSASVGDRVTITCRASQGISSALAWYQQKPGKAPQLLIYDASSLESGVPSRFSGSGSGTDFTLTISS |
| | | amino acid | LQPEDFATYYCQQFNSYPLTFGGGTKVEIK |
| | | sequence | |
| 37 | IMPI-045 | LCDR1 | QGISSA |
| 28 | IMPI-045 | LCDR2 | DAS |
| 47 | IMPI-045 | LCDR3 | QQFNSYPLT |
| 104 | IMPI-005 | VH domain | GAGGTGCAGCTGGTGGAGTCTGGAGGAGGCTTGATCCAGCCTGGGGGGTCCCTGAGACTCTCCTGTGCAGCCTCTGG |
| | | nucleic | GTTCACCGTCAGTAGCAACTATATGAACTGGGTCCGCCAGGCTCCAGGGAAGGGGCTGGAGTGGGTCTCAGTTATTT |
| | | acid | ATAGCGGTGGTAGCACATACTACGCAGACTCCGTGAAGGGCCGATTCACCATCTCCAGAGACAATTCCAAGAACACG |
| | | sequence | CTGTTTCTTCAAATGAACAGCCTGAGAGCCGAGGACTCGGCCGTGTATTACTGTGCGAGAGATTTGGGACCCTACGG |
| | | | TGTGGACGTCTGGGGCCAAGGGACCACGGTCACCGTCTCCTCA |
| 105 | IMPI-005 | VH domain | EVQLVESGGGLIQPGGSLRLSCAASGFTVSSNYMNWVRQAPGKGLEWVSVIYSGGSTYYADSVKGRFTISRDNSKNT |
| | | amino acid | LFLQMNSLRAEDSAVYYCARDLGPYGVDVWGQGTTVTVSS |
| | | sequence | |
| 13 | IMPI-005 | HCDR1 | GFTVSSNY |
| 14 | IMPI-005 | HCDR2 | IYSGGST |
| 106 | IMPI-005 | HCDR3 | ARDLGPYGVDV |
| 107 | IMPI-005 | VL domain | GACATCCAGTTGACCCAGTCTCCATCCTTCCTGTCTGCATCTGTAGGAGACAGAGTCACCATCACTTGCTGGGCCAG |
| | | nucleic | TCAGGGCATTAGCAGTTATTTAGCCTGGTATCAGCAAAAACCAGGGAAAGCCCCTAAGCTCCTGATCTATGCTGCAT |
| | | acid | CCACTTTGCAAAGTGGGGTCCCATCAAGGTTCAGCGGCAGTGGATCTGGGACAGAATTCACTCTCACAATCAGCAGC |
| | | sequence | CTGCAGCCTGAAGATTTTGCAACTTATTACTGTCAAGAGCTTAATAGTTACCCTCTCACCTTCGGCCAAGGGACACG |
| | | | ACTGGAGATTAAA |
| 108 | IMPI-005 | VL domain | DIQLTQSPSFLSASVGDRVTITCWASQGISSYLAWYQQKPGKAPKLLIYAASTLQSGVPSRFSGSGSGTEFTLTISS |
| | | amino acid | LQPEDFATYYCQELNSYPLTFGQGTRLEIK |
| | | sequence | |
| 8 | IMPI-005 | LCDR1 | QGISSY |
| 18 | IMPI-005 | LCDR2 | AAS |
| 109 | IMPI-005 | LCDR3 | QELNSYPLT |
| 110 | IMPI-038 | VH domain | CAGGTGCAGCTGGTGGAGTCTGGGGGAGGCGTGGTCCAGCCTGGGAGGTCCCTGAGACTCTCCTGTGCAGCCTCTGG |
| | | nucleic | ATTCACCTTCAGTAGCTATGGCATGCACTGGGTCCGCCAGGCTCCAGGCAAGGGGCTGGAGTGGGTGGCAGTTATAT |
| | | acid | CATATGATGGAAGTGATAAATACTATGCAGACTCCGTGAAGGGCCGATTCACCATCTCCAGAGACAATTCCAAGAAC |
| | | sequence | ACGCTGTATCTGCAAATGAACAGCCTGAGAGCTGAGGACACGGCTGTGTATTACTGTGCGAAAACAGTGGCTGGTTA |
| | | | TTACTACTACTACTACGGTATGGACGTCTGGGGCCAAGGGACCACGGTCACCGTCTCCTCA |
| 111 | IMPI-038 | VH domain | QVQLVESGGGVVQPGRSLRLSCAASGFTFSSYGMHWVRQAPGKGLEWVAVISYDGSDKYYADSVKGRFTISRDNSKN |
| | | amino acid | TLYLQMNSLRAEDTAVYYCAKTVAGYYYYYYGMDVWGQGTTVTVSS |
| | | sequence | |
| 112 | IMPI-038 | HCDR1 | GFTFSSYG |
| 113 | IMPI-038 | HCDR2 | ISYDGSDK |
| 114 | IMPI-038 | HCDR3 | AKTVAGYYYYYYGMDV |
| 115 | IMPI-038 | VL domain | GACATCCAGATGACCCAGTCTCCATCTTCCGTGTCTGCATCTGTTGGAGACAGAGTCACCGTCACTTGTCGGGCGAG |
| | | nucleic | TCAGGATATTAGTAGCTGGTTAGCCTGGTTTCAGCAGAAACCAGGGAAAGCCCCTAAATTCCTGATCTATGATGCAT |
| | | acid | CCAATTTGGAAAATGGAGTCCCATCAAGGTTCAGTGGCAGTGGATCTGGGACAGATTTCACCCTCACCATCAGCAGC |
| | | sequence | CTGCAGCCTGAAGATTTTGCAACTTACTATTGTCAACAAGCTAAAAGTTTCCCGTGGACGTTCGGCCAAGGGACCAA |
| | | | GGTGGAAATCAAC |
| 116 | IMPI-038 | VL domain | DIQMTQSPSSVSASVGDRVTVTCRASQDISSWLAWFQQKPGKAPKFLIYDASNLENGVPSRFSGSGSGTDFTLTISS |
| | | amino acid | LQPEDFATYYCQQAKSFPWTFGQGTKVEIN |
| | | sequence | |
| 117 | IMPI-038 | LCDR1 | QDISSW |
| 28 | IMPI-038 | LCDR2 | DAS |
| 118 | IMPI-038 | LCDR3 | QQAKSFPWT |
| 119 | IMPI-036 | VH domain | CAGGTGCAGCTGGTGCAGTCTGGGGCTGAGGTGAAGAAGCCTGGGGCCTCAGTGAAGGTCTCCTGCAAGGCTTCTGG |
| | | nucleic | ATACACCTTCACCAGTTATGATATCAACTGGGTGCGACAGGCCACTGGCCAAGGGCTTGAGTGGATGGGATGGATGA |
| | | acid | ACCCTATCAGTGGTAACACAGGCTATGCACAGAAGTTCCAGGGCAGAGTCACCATGACCAGAAACACCTCCATAAGC |
| | | sequence | ACAGCCTACATGGAGCTGAACAGCCTGAGATCTGAGGACACGGCCGTGTATTTCTGTGCGAGAGGCGGGCGATATTG |
| | | | TAGTGGTGTTAGCTGCTACTCCGGAGAGCCTTTTGACTACTGGGGCCAGGGAACCCTGGTCACCGTCTCCTCA |
| 120 | IMPI-036 | VH domain | QVQLVQSGAEVKKPGASVKVSCKASGYTFTSYDINWVRQATGQGLEWMGWMNPISGNTGYAQKFQGRVTMTRNTSIS |
| | | amino acid | TAYMELNSLRSEDTAVYFCARGGRYCSGVSCYSGEPFDYWGQGTLVTVSS |
| | | sequence | |
| 121 | IMPI-036 | HCDR1 | GYTFTSYD |
| 122 | IMPI-036 | HCDR2 | MNPISGNT |
| 123 | IMPI-036 | HCDR3 | ARGGRYCSGVSCYSGEPFDY |
| 124 | IMPI-036 | VL domain | GAAATTGTGCTGACTCAGTCTCCAGACTTTCAGTCTGTGACTCCAAAGGAGAAAGTCACCATCACCTGCCGGGCCAG |
| | | nucleic | TCAGAGCATTGGTAGTAGCTTACACTGGTACCAGCAGAAACCAGATCAGTCTCCAAAGCTCCTCATCAAGTATGCTT |
| | | acid | CCCAGTCCATCTCAGGGGTCCCCTCGAGGTTCAGTGGCAGTGGATCTGGGACAGATTTCACCCTCACCATCAATAGC |
| | | sequence | CTGGAAGCTGAAGATGCTGCAGCGTATTATTGTCATCAGAGTAGTAGTTTACCTCACACTTTCGGCCCTGGGACCAA |
| | | | AGTGGAGATCAAA |
| 125 | IMPI-036 | VL domain | EIVLTQSPDFQSVTPKEKVTITCRASQSIGSSLHWYQQKPDQSPKLLIKYASQSISGVPSRFSGSGSGTDFTLTINS |
| | | amino acid | LEAEDAAAYYCHQSSSLPHTFGPGTKVEIK |
| | | sequence | |
| 126 | IMPI-036 | LCDR1 | QSIGSS |
| 127 | IMPI-036 | LCDR2 | YAS |
| 128 | IMPI-036 | LCDR3 | HQSSSLPHT |
| 129 | IMPI-023 | VH domain | CAGGTGCAGCTGCAGGAGTCGGGCCCAGGACTGGTGAAGCCTTCGGGGACCCTGTCCCTCACCTGCGCTGTCTCTGG |
| | | nucleic | TGGCTCCATCAGCAGTAATAACTGGTGGAGTTGGGTCCGCCAGCCCCCAGGGAAGGGTCTGGAGTGGATTGGGGAAA |
| | | acid | TCGATCATAGTGGGAGCACCATGTACAACCCGTCCCTCAAGAGTCGAGTCACCATATCAGTAGACAAGTCCAAGAAC |
| | | sequence | CAGTTCTCCCTGAAGCTGAACTCTGTGACCGCCGCGGACACGGCCGTGTATTACTGTACGGGAGAGGGGTATAACTG |
| | | | GAACTACTGGGGCCAGGGGACCCTGGTCACCGTCTCCTCA |
| 130 | IMPI-023 | VH domain | QVQLQESGPGLVKPSGTLSLTCAVSGGSISSNNWWSWVRQPPGKGLEWIGEIDHSGSTMYNPSLKSRVTISVDKSKN |
| | | amino acid | QFSLKLNSVTAADTAVYYCTGEGYNWNYWGQGTLVTVSS |
| | | sequence | |
| 131 | IMPI-023 | HCDR1 | GGSISSNNW |
| 132 | IMPI-023 | HCDR2 | IDHSGST |
| 133 | IMPI-023 | HCDR3 | TGEGYNWNY |
| 134 | IMPI-023 | VL domain | GTTGTTGTGATGACTCAGTCTCCACTCTCCCTGCCCGTCACCCTTGGACAGCCGGCCTCCATCTCCTGCAGGTCTAG |
| | | nucleic | TCAAAGCCTCCTATACACTGATGGAAACACCTACTTGAGTTGGTTTCAGCAGAGGCCAGGCCAATCTCCAAGGCGCC |
| | | acid | TAATTTATAAGGTTTCTAACTGGGACTCTGGGGTCCCAGACAGATTCAGCGGCAGTGGGTCAGGCACTGATTTCACA |
| | | sequence | CTGAAAATCAGCAGGGTGGAGGCTGAAGATGTTGGGATTTATTACTGCATGCAAGGTACACACTGGCCGCTCACTTT |
| | | | CGGCGGAGGGACCAAGGTGGAGATCAAA |
| 135 | IMPI-023 | VL domain | VVVMTQSPLSLPVTLGQPASISCRSSQSLLYTDGNTYLSWFQQRPGQSPRRLIYKVSNWDSGVPDRFSGSGSGTDFT |
| | | amino acid | LKISRVEAEDVGIYYCMQGTHWPLTFGGGTKVEIK |
| | | sequence | |
| 136 | IMPI-023 | LCDR1 | QSLLYTDGNTY |
| 85 | IMPI-023 | LCDR2 | KVS |
| 86 | IMPI-023 | LCDR3 | MQGTHWPLT |
| 137 | IMPI-019 | VH domain | CAGGTGCAGCTGCAGGAGTCGGGCCCAGGACTGATAAAGCCTTCGGGGACCCTGTCCCTCACCTGCGCTGTCTCTGG |
| | | nucleic | TGGCTCCATCAGCAGTAATACCTGGTGGAGTTGGGTCCGCCAGCCCCCAGGGAAGGGTCTGGAGTGGATTGGGGAAA |
| | | acid | TCTATCATAGTGGGAGCACCATGTACAACCCGTCCCTCAAGAGTCGAGTCACCATATCAGTAGACAAGTCCAAGAAC |
| | | sequence | CAGTTCTCCCTGAAGTTGAACTCTGTGACCGCCGCGGACACGGCCGTGTATTACTGTACGGGAGAGGGGCATAACTG |
| | | | GAACTACTGGGGCCAGGGGACCCTGGTCACCGTCTCCTCA |
| 138 | IMPI-019 | VH domain | QVQLQESGPGLIKPSGTLSLTCAVSGGSISSNTWWSWVRQPPGKGLEWIGEIYHSGSTMYNPSLKSRVTISVDKSKN |
| | | amino acid | QFSLKLNSVTAADTAVYYCTGEGHNWNYWGQGTLVTVSS |
| | | sequence | |
| 139 | IMPI-019 | HCDR1 | GGSISSNTW |
| 140 | IMPI-019 | HCDR2 | IYHSGST |
| 141 | IMPI-019 | HCDR3 | TGEGHNWNY |
| 142 | IMPI-019 | VL domain | GATGTTGTGTTGACTCAGTCTCCACTCTCCCTGCCCGTCACCCTTGGACAGCCGGCCTCCATCTCCTGCAGGTCTAG |
| | | nucleic | TCAAAGCCTCGTATACACTGATGGAAACACCTACTTGAGTTGGTTTCAGCAGAGGCCAGGCCAATCTCCAAGGCGCC |
| | | acid | TAATTTATAAGGTTTCTAACTGGGAATCTGGGGTCCCAGACAGATTCAGCGGCAGTGGGTCAGGCACTGATTTCACA |
| | | sequence | CTGAAAATCAGCAGGGTGGAGGCTGAAGATGTTGGGATTTATTACTGCATGCAAGGTACACACTGGCCGCTCACTTT |
| | | | CGGCGGAGGGACCAAGGTGGAGATCAAA |
| 143 | IMPI-019 | VL domain | DVVLTQSPLSLPVTLGQPASISCRSSQSLVYTDGNTYLSWFQQRPGQSPRRLIYKVSNWESGVPDRFSGSGSGTDFT |
| | | amino acid | LKISRVEAEDVGIYYCMQGTHWPLTFGGGTKVEIK |
| | | sequence | |
| 144 | IMPI-019 | LCDR1 | QSLVYTDGNTY |
| 85 | IMPI-019 | LCDR2 | KVS |
| 86 | IMPI-019 | LCDR3 | MQGTHWPLT |
| 145 | IMPI-008 | VH domain | GAAGTGCAGCTGGTGGAGTCTGGGGGAGGCTTGGTACAGCCTGGCAGGTCCCTGAGACTCTCCTGTGCAGCCTCTGG |
| | | nucleic | ATTCACCTTTGATGATTATGCCATGCACTGGGTCCGGCAAGCTCCAGGGAAGGGCCTGGAGTGGGTCTCAGGTATTA |
| | | acid | GTTGGAATAGTGGTAGCATAGGTTATGCGGACTCTGTGAAGGGCCGATTCACCATCTCCAGAGACAACGCCAAGAAC |
| | | sequence | TCCCTGTATCTGCAAATGAACAGTCTGAGAGTTGAGGACACGGCCTTGTATTACTGTGCAAAAGATCTCCAGGGGGA |
| | | | CTACTACTACTACGGTGTGGACGTCTGGGGCCAAGGGACCACGGTCACCGTCTCCTCA |
| 146 | IMPI-008 | VH domain | EVQLVESGGGLVQPGRSLRLSCAASGFTFDDYAMHWVRQAPGKGLEWVSGISWNSGSIGYADSVKGRFTISRDNAKN |
| | | amino acid | SLYLQMNSLRVEDTALYYCAKDLQGDYYYYGVDVWGQGTTVTVSS |
| | | sequence | |
| 22 | IMPI-008 | HCDR1 | GFTFDDYA |
| 23 | IMPI-008 | HCDR2 | ISWNSGSI |
| 147 | IMPI-008 | HCDR3 | AKDLQGDYYYYGVDV |
| 148 | IMPI-008 | VL domain | GCCATCCAGTTGACCCAGTCTCCATCCTCCCTGTCTGCATCTGTAGGAGACAGAGTCACCATCACTTGCCGGGCAAG |
| | | nucleic | TCAGGGCATTAGCAGTGCTTTAGCCTGGTATCAGCAGAAACCAGGGAAACCTCCTAAGCTCCTGATCTATGATGCCT |
| | | acid | CCAGTTTGGTAAGTGGGGTCCCATCAAGATTCAGCGGCAGTAGATCTGGGACAGATTTCACTCTCACCATCAGCAGC |
| | | sequence | CTGCAACCTGAGGATTTTGCAACTTATTACTGTCAACAGTTTATTAGTTACCCGCTCACTTTCGGCGGAGGGACCAG |
| | | | GGTGGAGATCAAA |
| 149 | IMPI-008 | VL domain | AIQLTQSPSSLSASVGDRVTITCRASQGISSALAWYQQKPGKPPKLLIYDASSLVSGVPSRFSGSRSGTDFTLTISS |
| | | amino acid | LQPEDFATYYCQQFISYPLTFGGGTRVEIK |
| | | sequence | |
| 37 | IMPI-008 | LCDR1 | QGISSA |
| 28 | IMPI-008 | LCDR2 | DAS |
| 69 | IMPI-008 | LCDR3 | QQFISYPLT |
| 150 | IMPI-004 | VH domain | CAGGTGCAGCTGGTGGAGTCTGGGGGAGGCGTGGTCCAGCCTGGGAGGTCCCTGAGACTCTCCTGTGCAGCGTCTGG |
| | | nucleic | ATTCACCTTCAGTAGCTATGGCATGCACTGGGTCCGCCAGGCTCCAGGCAAGGGGCTGGAGTGGGTGGCAGTTATAT |
| | | acid | GGTATGATGGAGGTAATAAATACTATGCAGACTCCGTGAAGGGCCGATTCACCATCTCCAGAGACAATTCTAAGAAC |
| | | sequence | ACGCTGTATCTGCAAATGAACAGCCTGAGAGCCGAGGACACGGCTGTGTATTACTGTGCGAGAGTTAATGTACTAAT |
| | | | GGGCTACGGTATGGACGTCTGGGGCCAAGGGACCACGGTCACCGTCTCCTCA |
| 151 | IMPI-004 | VH domain | QVQLVESGGGVVQPGRSLRLSCAASGFTFSSYGMHWVRQAPGKGLEWVAVIWYDGGNKYYADSVKGRFTISRDNSKN |
| | | amino acid | TLYLQMNSLRAEDTAVYYCARVNVLMGYGMDVWGQGTTVTVSS |
| | | sequence | |
| 112 | IMPI-004 | HCDR1 | GFTFSSYG |
| 152 | IMPI-004 | HCDR2 | IWYDGGNK |
| 153 | IMPI-004 | HCDR3 | ARVNVLMGYGMDV |
| 154 | IMPI-004 | VL domain | GAAATTGTGTTGACGCAGTCTCCAGGCACCCTGTCTTTGTCTCCAGGGGATAAAGCCACCCTCTCCTGCAGGGCCAG |
| | | nucleic | TCAGAGTATCATCAGCAGCTTCTTAGCCTGGTACCAGCAGAAACCTGGCCAGGCTCCCAGGCTCCTCATCTATGGTG |
| | | acid | CATCCAGCAGGGCCACTGGCATCCCAGACAGGTTCAGTGGCAGTGGGTCTGGGACAGACTTCACTCTCACCATCAGC |
| | | sequence | AGACTGGAACCTGAAGATTTTGCAGTGTATTACTGTCAGCAGTATGGTAGCTCGCTCACTTTCGGCGGAGGGACCAA |
| | | | GGTGGAGATCAAA |
| 155 | IMPI-004 | VL domain | EIVLTQSPGTLSLSPGDKATLSCRASQSIISSFLAWYQQKPGQAPRLLIYGASSRATGIPDRFSGSGSGTDFTLTIS |
| | | amino acid | RLEPEDFAVYYCQQYGSSLTFGGGTKVEIK |
| | | sequence | |
| 156 | IMPI-004 | LCDR1 | QSIISSF |
| 157 | IMPI-004 | LCDR2 | GAS |
| 158 | IMPI-004 | LCDR3 | QQYGSSLT |
| 159 | IMPI-012 | VH domain | GAGGTGCAGCTGGTGGAGTCTGGAGGAGGCTTGATCCAGCCTGGGGGGTCCCTGAGACTCTCCTGTGCAGCCTCTGG |
| | | nucleic | GATCACCGTCAGTAGCAACTACATGAGCTGGGTCCGCCAGGCTCCAGGGAAGGGGCTGGAGTGGGTCTCAGTTATTT |
| | | acid | ATAGCGGTGGTACTACAAACTACGCAGACTCCGTGAAGGGCCGATTCACCATCTCCAGAGACAATTCCAAGAACACG |
| | | sequence | CTGTATCTTCAAATGAACAGCCTGAGAGCCGAGGACACGGCCGTGTATTACTGTGCGAGAGATACGGTGGTCTACGG |
| | | | TATGGACGTCTGGGGCCAAGGGACCACGGTCACCGTCTCCTCA |
| 160 | IMPI-012 | VH domain | EVQLVESGGGLIQPGGSLRLSCAASGITVSSNYMSWVRQAPGKGLEWVSVIYSGGTTNYADSVKGRFTISRDNSKNT |
| | | amino acid | LYLQMNSLRAEDTAVYYCARDTVVYGMDVWGQGTTVTVSS |
| | | sequence | |
| 161 | IMPI-012 | HCDR1 | GITVSSNY |
| 4 | IMPI-012 | HCDR2 | IYSGGTT |
| 5 | IMPI-012 | HCDR3 | ARDTVVYGMDV |
| 162 | IMPI-012 | VL domain | GACATCCAGTTGACCCAGTCTCCATCCTTCCTGTCTGCATCTGTAGGAGACAGAGTCACCATCACTTGCTGGGCCAG |
| | | nucleic | TCAGGGCATTAGCAGTTATTTAGCCTGGTATCAGCAAAAACCAGGGAAAGCCCCTAAGCTCCTGATCTATGCTGCAT |
| | | acid | CCACTTTGCAAAGTGGGGTCCCATCAAGGTTCAGCGGCAGTGGATCTGGGACAGAATTCACTCTCACAATCAGCAGC |
| | | sequence | CTGCAGCCTGAAGATTTTGCAACTTATTACTGTCAACAGCTTAATAGTCACCCGCTCACTTTCGGCGGAGGGACCAA |
| | | | GGTGGAGATCAAA |
| 163 | IMPI-012 | VL domain | DIQLTQSPSFLSASVGDRVTITCWASQGISSYLAWYQQKPGKAPKLLIYAASTLQSGVPSRFSGSGSGTEFTLTISS |
| | | amino acid | LQPEDFATYYCQQLNSHPLTFGGGTKVEIK |
| | | sequence | |
| 8 | IMPI-012 | LCDR1 | QGISSY |
| 18 | IMPI-012 | LCDR2 | AAS |
| 164 | IMPI-012 | LCDR3 | QQLNSHPLT |
| 165 | IMPI-010 | VH domain | GAAGTGCAGCTGGTGGAGTCTGGGGGAGGCTTGGTACAGCCTGGCAGGTCCCTGAGACTCTCCTGTGCAGCCTCTGG |
| | | nucleic | ATTCACCTTTGATGATTATGCCATGCACTGGGTCCGGCAAGCTCCAGGGAAGGGCCTGGAGTGGGTCTCAGGTATTA |
| | | acid | GTTGGAATAGTGGTAGCATAGGCTATGCGGACTCTGTGAAGGGCCGATTCACCATCTCCAGAGACAACGCCAAGAAC |
| | | sequence | TCCCTGTATCTGCAAATGAACAGTCTGAGAGCTGAGGACACGGCCTTGTATTACTGTGCAAAAGATCTCCAGGGGGA |
| | | | CGACTACTACTACGGTGTGGACGTCTGGGGCCAAGGGACCACGGTCACCGTCTCCTCA |
| 166 | IMPI-010 | VH domain | EVQLVESGGGLVQPGRSLRLSCAASGFTFDDYAMHWVRQAPGKGLEWVSGISWNSGSIGYADSVKGRFTISRDNAKN |
| | | amino acid | SLYLQMNSLRAEDTALYYCAKDLQGDDYYYGVDVWGQGTTVTVSS |
| | | sequence | |
| 22 | IMPI-010 | HCDR1 | GFTFDDYA |
| 23 | IMPI-010 | HCDR2 | ISWNSGSI |
| 167 | IMPI-010 | HCDR3 | AKDLQGDDYYYGVDV |
| 168 | IMPI-010 | VL domain | GCCATCCAGTTGACCCAGTCTCCATCCTCCCTGTCTGCATCTGTAGGAGACAGAGTCACCATCACTTGCCGGGCAAG |
| | | nucleic | TCAGGACATTAGCAGTGCTTTAGCCTGGTATCAGCAGAAACCAGGGAAAGCTCCTAAGCTCCTGATCTATGATGCCT |
| | | acid | CCAGTTTGGAAAGTGGGGTCCCATCAAGGTTCAGCGGCAGTAGATCTGGGACAGATTTCACTCTCACCATCAGCAGC |
| | | sequence | CTGCAACCTGAAGATTTTGCAAATTATTACTGTCAACAGTTTATTAGTTACCCGCTCACTTTCGGCGGAGGGACCAA |
| | | | GGTGGAGATCAAA |
| 169 | IMPI-010 | VL domain | AIQLTQSPSSLSASVGDRVTITCRASQDISSALAWYQQKPGKAPKLLIYDASSLESGVPSRFSGSRSGTDFTLTISS |
| | | amino acid | LQPEDFANYYCQQFISYPLTFGGGTKVEIK |
| | | sequence | |
| 67 | IMPI-010 | LCDR1 | QDISSA |
| 28 | IMPI-010 | LCDR2 | DAS |
| 69 | IMPI-010 | LCDR3 | QQFISYPLT |
| 170 | IMPI-017 | VH domain | GAGGTGCAGCTGGTGGAGTCTGGAGGAGGCTTGATCCAGCCGGGGGGGTCCCTGAGACTCTCCTGTGCAGCCGCTGG |
| | | nucleic | GTTAACCGTCAGTAGCAACTATATGAACTGGGTCCGCCAGGTTCCAGGGAAGGGGCTGGAGTGGGTCTCAGTTATTT |
| | | acid | ATAGCGGTGGTAGCACATACTACGCAGACTCCGTGAAGGGCCGATTCACCATCTCCAGAGACAATTCCAAGAACACG |
| | | sequence | CTGTATCTTCAAATGAACAGTCTGAGAGCCGAGGACACGGCCGTGTATTACTGTGCGAGAGATCTGGGTACCCTGGG |
| | | | TATGGACGTCTGGGGCCAAGGGACCACGGTCACCGTCTCCTCA |
| 171 | IMPI-017 | VH domain | EVQLVESGGGLIQPGGSLRLSCAAAGLTVSSNYMNWVRQVPGKGLEWVSVIYSGGSTYYADSVKGRFTISRDNSKNT |
| | | amino acid | LYLQMNSLRAEDTAVYYCARDLGTLGMDVWGQGTTVTVSS |
| | | sequence | |
| 172 | IMPI-017 | HCDR1 | GLTVSSNY |
| 14 | IMPI-017 | HCDR2 | IYSGGST |
| 173 | IMPI-017 | HCDR3 | ARDLGTLGMDV |
| 174 | IMPI-017 | VL domain | GACATCCAGTTGACCCAGTCTCCATCCTTCCTGTCTGCATCTGTAGGAGACAGAGTCACCATCACTTGCTGGGCCAG |
| | | nucleic | TCAGGGCATTAGCACTTATTTAGCCTGGTATCAGCAAAAACCAGGGAAAGCCCCTAAGCTCCTGATCTATGCTGCAT |
| | | acid | CCACTTTGCAGAGCGGGGTCCCATCAAGGTTCAGCGGCAGTGGATCTGGGACAGAATTCACTCTCACAATCAGCAGC |
| | | sequence | CTGCAGCCTGAAGATTTTGCAACTTATTACTGTCAGCAGCTTAACAGTTACCTTCCGACGTTCGGCCAAGGGACCAA |
| | | | GGTGGAAATCAAA |
| 175 | IMPI-017 | VL domain | DIQLTQSPSFLSASVGDRVTITCWASQGISTYLAWYQQKPGKAPKLLIYAASTLQSGVPSRFSGSGSGTEFTLTISS |
| | | amino acid | LQPEDFATYYCQQLNSYLPTFGQGTKVEIK |
| | | sequence | |
| 176 | IMPI-017 | LCDR1 | QGISTY |
| 18 | IMPI-017 | LCDR2 | AAS |
| 177 | IMPI-017 | LCDR3 | QQLNSYLPT |
| 178 | IMPI-037 | VH domain | GAGGTGCAGCTGGTGGAGTCTGGGGGAGGCTTGGTAAAGCCTGGGGGGTCCCTTAGACTCTCCTGTGCAGCCTCTGG |
| | | nucleic | ATTCACTTTCAGTAACGCCTGGATGAGCTGGGTCCGCCAGGCTCCAGGGAAGGGGCTGGAGTGGGTTGGCCGTATTA |
| | | acid | AAAACAAAGCTGATGGTGGGACAACAGACTACGCTGCACCCGTGAAAGGCAGATTCACCATCTCAAGAGATGATTCA |
| | | sequence | AAAAACACGTTGTATCTGCAAATGAACAGCCTGAAAACCGAGGACACAGCCGTGTATTACTGTACCACAGAGGAATT |
| | | | ACTATGGTTCGGGGAGTCGCCCTTTGACTGCTGGGGCCAGGGAACCCTGGTCACCGTCTCCTCA |
| 179 | IMPI-037 | VH domain | EVQLVESGGGLVKPGGSLRLSCAASGFTFSNAWMSWVRQAPGKGLEWVGRIKNKADGGTTDYAAPVKGRFTISRDDS |
| | | amino acid | KNTLYLQMNSLKTEDTAVYYCTTEELLWFGESPFDCWGQGTLVTVSS |
| | | sequence | |
| 180 | IMPI-037 | HCDR1 | GFTFSNAW |
| 181 | IMPI-037 | HCDR2 | IKNKADGGTT |
| 182 | IMPI-037 | HCDR3 | TTEELLWFGESPFDC |
| 183 | IMPI-037 | VL domain | GACATCCAGTTGACCCAGTCTCCATCCTTCCTGTCTGCATCTGTAGGAGACAGAGTCACCATCACTTGCTGGGCCAG |
| | | nucleic | TCAGGGCATTAACAGTTATTTAGCCTGGTATCAGCAAAAACCAGGGAAAGCCCCTAAGCTCCTGATCTATGCTGCAT |
| | | acid | CCACTTTGCAAAGTGGGGTCCCATCAAGGTTCAGCGGCAGTGGATCTGGGACAGAATTCACTCTCACAATCAGCAGC |
| | | sequence | CTGCAGCCTGAAGATTTTACAACTTATTACTGTCAACAATTTAATGATTACCCTCGGACGTTCGGCCCAGGGACCAA |
| | | | GGTGGAAATCAAA |
| 184 | IMPI-037 | VL domain | DIQLTQSPSFLSASVGDRVTITCWASQGINSYLAWYQQKPGKAPKLLIYAASTLQSGVPSRFSGSGSGTEFTLTISS |
| | | amino acid | LQPEDFTTYYCQQFNDYPRTFGPGTKVEIK |
| | | sequence | |
| 185 | IMPI-037 | LCDR1 | QGINSY |
| 18 | IMPI-037 | LCDR2 | AAS |
| 186 | IMPI-037 | LCDR3 | QQFNDYPRT |
| 187 | IMPI-022 | VH domain | GAAGTGCAGCTGGTGGAGTCTGGGGGAGGCTTGGTACAGCCTGGCAGGTCCCTGAGACTCTCCTGTGCAGCCTCTGG |
| | | nucleic | ATTCACCTTTGATGATTATGCCATGCACTGGGTCCGGCAAGCTCCAGGGAAGGGCCTGGAGTGGGTCTCAGGTATTA |
| | | acid | CTTGGAATAGTGGTAGCATAGGTTATGCGGACTCTGTGAAGGGCCGATTCACCATCTCCAGAGACAACGCCAAGAAC |
| | | sequence | TCCCTGTATCTGCACATGAACAGTCTGAGAGTTGAGGACACGGCCTTGTATTACTGTGCAAAAGATCTCCAGGGGGA |
| | | | CTACTACTACTACGGTGTGGACGTCTGGGGCCAAGGGACCACGGTCACCGTCTCCTCA |
| 188 | IMPI-022 | VH domain | EVQLVESGGGLVQPGRSLRLSCAASGFTFDDYAMHWVRQAPGKGLEWVSGITWNSGSIGYADSVKGRFTISRDNAKN |
| | | amino acid | SLYLHMNSLRVEDTALYYCAKDLQGDYYYYGVDVWGQGTTVTVSS |
| | | sequence | |
| 22 | IMPI-022 | HCDR1 | GFTFDDYA |
| 63 | IMPI-022 | HCDR2 | ITWNSGSI |
| 147 | IMPI-022 | HCDR3 | AKDLQGDYYYYGVDV |
| 189 | IMPI-022 | VL domain | GCCATCCAGTTGACCCAGTCTCCATCCTCCCTGTCTGCATCTGTAGGAGACAGAGTCACCATCACTTGCCGGGCAAG |
| | | nucleic | TCAGGGCATTAGCAGTGCTTTAGCCTGGTATCAGCAGAAGCCAGGGAAAGCTCCTAAACTCCTGCTCTATGATGCCT |
| | | acid | CCAGTTTGGTAAGTGGGGTCCCATCAAGATTCAGCGGCAGTAGATCTGGGACAGATTTCACTCTCACCATCAGCAGC |
| | | sequence | CTGCAACCTGAGGATTTTGCAACTTATTACTGTCAACAGTTTATTAGTTACCCGCTCACTTTCGGCGGAGGGACCAG |
| | | | GGTGGAGATCAAA |
| 190 | IMPI-022 | VL domain | AIQLTQSPSSLSASVGDRVTITCRASQGISSALAWYQQKPGKAPKLLLYDASSLVSGVPSRFSGSRSGTDFTLTISS |
| | | amino acid | LQPEDFATYYCQQFISYPLTFGGGTRVEIK |
| | | sequence | |
| 37 | IMPI-022 | LCDR1 | QGISSA |
| 28 | IMPI-022 | LCDR2 | DAS |
| 69 | IMPI-022 | LCDR3 | QQFISYPLT |
| 191 | IMPI-058 | VH domain | GAGGTGCAGCTGGTGCAGTCTGGAGCAGAGGTGAAAAAGCCCGGGGAGTCTCTGAAGATCTCCTGTAAGGGTTCTGG |
| | | nucleic | ATACAGCTTTAGCAGCTACTGGATCGGCTGGGTGCGCCAGATGCCCGGGAAAGGCCTGGAGTGGATGGGGATCATCT |
| | | acid | ATCCTGGTGACTCTGAAACCAGATACAGCCCGTCCTTCCAAGGCCAGGTCACCATCTCAGCCGACAAGTCCATCAGC |
| | | sequence | ACCGCCTACCTGCAGTGGAGCAGCCTGAAGGCCTCGGACACCGCCATGTATTACTGTGCGAGGTCGTATAACTGGAA |
| | | | CTACTTTGACTACTGGGGCCAGGGAACCCTGGTCACCGTCTCCTCA |
| 192 | IMPI-058 | VH domain | EVQLVQSGAEVKKPGESLKISCKGSGYSFSSYWIGWVRQMPGKGLEWMGIIYPGDSETRYSPSFQGQVTISADKSIS |
| | | amino acid | TAYLQWSSLKASDTAMYYCARSYNWNYFDYWGQGTLVTVSS |
| | | sequence | |
| 193 | IMPI-058 | HCDR1 | GYSFSSYW |
| 194 | IMPI-058 | HCDR2 | IYPGDSET |
| 34 | IMPI-058 | HCDR3 | ARSYNWNYFDY |
| 195 | IMPI-058 | VL domain | GCCATCCAGTTGACCCAGTCTCCATCCTCCCTGTCTGCATCTGTAGGGGACAGAGTCATCATCACTTGCCGGGCAAG |
| | | nucleic | TCAGGGCATTAGCAGTGCTTTAGCCTGGTATCAGCAGAAACCAGGGAAAGCTCTTAAACTCCTGATCTATGATGCCT |
| | | acid | CCAGTTTGGAAAGTGGGGTCCCATCAAGGTTCAGCGGCAGTGGATCTGGGACAGATTTCACTCTCACCATCACCAGC |
| | | sequence | CTGCAGCCTGAAGATTTTGCAACTTATTACTGTCAACAGTTTAGTAGTTACCCTTGGACGTTCGGCCAAGGGACCAA |
| | | | GGTGGAAATCAAA |
| 196 | IMPI-058 | VL domain | AIQLTQSPSSLSASVGDRVIITCRASQGISSALAWYQQKPGKALKLLIYDASSLESGVPSRFSGSGSGTDFTLTITS |
| | | amino acid | LQPEDFATYYCQQFSSYPWTFGQGTKVEIK |
| | | sequence | |
| 37 | IMPI-058 | LCDR1 | QGISSA |
| 28 | IMPI-058 | LCDR2 | DAS |
| 197 | IMPI-058 | LCDR3 | QQFSSYPWT |
| 198 | IMPI-024 | VH domain | GAGGTGCAGCTGGTGCAGTCTGGAGCAGAGGTGAAAAAGCCCGGGGAGTCTCTGAAGATCTCCTGTAAGGGTTCTGG |
| | | nucleic | ATACAGCTTTACCAGCTACTGGATCGGCTGGGTGCGCCAGATGCCCGGGAAAGGCCTGGAGTGGATGGGGATCATCT |
| | | acid | ATCCTGGTGACTCTGATACCAGATACAGCCCGTCCTTCCAAGGCCAGGTCACCATCTCAGCCGACAAGTCCATCAGC |
| | | sequence | ACCGCCTACCTGCAGTGGAGCAGCCTGAAGGCCTCGGACACCGCCATGTATTACTGTGTGAGGTCGTATAATTGGAA |
| | | | CTACTTTGACTACTGGGGCCAGGGAACCCTGGTCACCGTCTCCTCA |
| 199 | IMPI-024 | VH domain | EVQLVQSGAEVKKPGESLKISCKGSGYSFTSYWIGWVRQMPGKGLEWMGIIYPGDSDTRYSPSFQGQVTISADKSIS |
| | | amino acid | TAYLQWSSLKASDTAMYYCVRSYNWNYFDYWGQGTLVTVSS |
| | | sequence | |
| 32 | IMPI-024 | HCDR1 | GYSFTSYW |
| 33 | IMPI-024 | HCDR2 | IYPGDSDT |
| 200 | IMPI-024 | HCDR3 | VRSYNWNYFDY |
| 201 | IMPI-024 | VL domain | GCCATCCAGTTGACCCAGTCTCCATCCTCCCTGTCTGCATCTGTAGGAGACAGAGTCACCATCACTTGCCGGGCAAG |
| | | nucleic | TCAGGGCATTAGCAGTGGTTTAGCCTGGTATCAGCAGAAAGCAGGGAAAGCTCCTAAGCTCCTGATCTATGATGTCT |
| | | acid | CCAGTTTGGAAAGTGGGGTCCCATCAAGGTTCAGCGGCAGTGGATCTGGGACAGATTTCACTCTCACCATCAGCAGC |
| | | sequence | CTGCAGCCTGAAGATTTTGCAACTTATTACTGTCAACAGTTTCTTAGTTACCCTTGGACGTTCGGCCAAGGGACCAA |
| | | | GGTGGAAATCAAA |
| 202 | IMPI-024 | VL domain | AIQLTQSPSSLSASVGDRVTITCRASQGISSGLAWYQQKAGKAPKLLIYDVSSLESGVPSRFSGSGSGTDFTLTISS |
| | | amino acid | LQPEDFATYYCQQFLSYPWTFGQGTKVEIK |
| | | sequence | |
| 92 | IMPI-024 | LCDR1 | QGISSG |
| 203 | IMPI-024 | LCDR2 | DVS |
| 204 | IMPI-024 | LCDR3 | QQFLSYPWT |
| 205 | IMPI-039 | VH domain | CAGGTGCAGCTGCAGGAGTCGGGCCCAGGACTGGTGAAGCCTTCGGAGACCCTGTCCCTCACCTGCGCTGTCTCGGG |
| | | nucleic | TGGCTCCATCAGCAGAAGTACCTGGTGGAGTTGGGTCCGCCAGCCCCCAGGGAAGGGGCTGGAGTGGATTGGGGAAA |
| | | acid | TCTCTCATAGTGGGAGTACCAAATACAACCCGTCCCTCAAGAGTCGAGTCACCATATCAGTAGACAAGTCCAAGAAC |
| | | sequence | CAGTTCTCCCTGAAGCTGAGCTCTGTGACCGCCGCGGACACGGCCGTATATTACTGTGCGGGAGAGGGGTCTAACTG |
| | | | GAGTTACTGGGGCCAGGGAACCCTGGTCACCGTCTCCTCA |
| 206 | IMPI-039 | VH domain | QVQLQESGPGLVKPSETLSLTCAVSGGSISRSTWWSWVRQPPGKGLEWIGEISHSGSTKYNPSLKSRVTISVDKSKN |
| | | amino acid | QFSLKLSSVTAADTAVYYCAGEGSNWSYWGQGTLVTVSS |
| | | sequence | |
| 79 | IMPI-039 | HCDR1 | GGSISRSTW |
| 80 | IMPI-039 | HCDR2 | ISHSGST |
| 207 | IMPI-039 | HCDR3 | AGEGSNWSY |
| 208 | IMPI-039 | VL domain | GATGTTGTGATGACTCAGTCTCCACACTCCCTGCCCGTCACCCTTGGACAGCCGGCCTCCATCTCCTGCAGGTCTAG |
| | | nucleic | TCAAAGCCTCGTATACAGTGATGGAAACACATATTTGAGTTGGTTTCAGCAGAGGCCAGGCCAATCTCCAAGGCGCC |
| | | acid | TAATTTATAAGGTTTCTAAATGGGACGCTGGGGTCCCAGACAGATTCAGCGGCAGTGGGTCAGGCACTGATTTCACA |
| | | sequence | CTGAAAATCAGCAGGGTGGAGGCTGAGGATATTGGGATTTATTACTGCATGCAAGGTACACACTGGCCGCTCACTTT |
| | | | CGGCGGAGGGACCAAGGTGGAGCTCAAA |
| 209 | IMPI-039 | VL domain | DVVMTQSPHSLPVTLGQPASISCRSSQSLVYSDGNTYLSWFQQRPGQSPRRLIYKVSKWDAGVPDRFSGSGSGTDFT |
| | | amino acid | LKISRVEAEDIGIYYCMQGTHWPLTFGGGTKVELK |
| | | sequence | |
| 84 | IMPI-039 | LCDR1 | QSLVYSDGNTY |
| 85 | IMPI-039 | LCDR2 | KVS |
| 86 | IMPI-039 | LCDR3 | MQGTHWPLT |
| 210 | IMPI-020 | VH domain | CAGGTGCAGCTGCAGGAGTCGGGCCCAGGACTGGTGAAGCCTTCGGGGACCCTGTCCCTCACCTGCGCTGTCTCTGG |
| | | nucleic | TGGCTCCATCAGCAGTAGTAACTGGTGGAGTTGGGTCCGCCAGCCCCCAGGGAAGGGGCTGGAGTGGATTGGGGAAA |
| | | acid | TCTATCATAGTGGGAGCACCATGTACAACCCGTCCCTCAAGAGTCGAGTCACCATATCAGTAGACAAGTCCAAGAAC |
| | | sequence | CAGTTCTCCCTGAAGCTGAGCTCTGTGACCGCCGCGGACACGGCCGTGTATTACTGTACGGGAGAGGGGTATAACTG |
| | | | GAACTACTGGGGCCAGGGAACCCTGGTCACCGTCTCCTCA |
| 211 | IMPI-020 | VH domain | QVQLQESGPGLVKPSGTLSLTCAVSGGSISSSNWWSWVRQPPGKGLEWIGEIYHSGSTMYNPSLKSRVTISVDKSKN |
| | | amino acid | QFSLKLSSVTAADTAVYYCTGEGYNWNYWGQGTLVTVSS |
| | | sequence | |
| 212 | IMPI-020 | HCDR1 | GGSISSSNW |
| 140 | IMPI-020 | HCDR2 | IYHSGST |
| 133 | IMPI-020 | HCDR3 | TGEGYNWNY |
| 213 | IMPI-020 | VL domain | GATGTTGTGTTGACTCAGTCTCCACTCTCCCTGCCCGTCACCCTTGGACAGCCGGCCTCCATCTCCTGCAGGTCTAG |
| | | nucleic | TCAAAGCCTCGTATACAGTGATGGAAACACCTACTTGAGTTGGTTTCAACAGAGGCCAGGCCAATCTCCAAGGCGCC |
| | | acid | TAATTTATAAGGTTTCTAACTGGGACTCTGGGGTCCCAGACAGATTCAGCGGCAGTGGGTCAGGCTCTGATTTCACA |
| | | sequence | CTGAAGATCAGCAGGGTGGAGGCTGAGGATGTTGGGGTTTATTACTGCATGCAAGGTACACACTGGCCGCTCACTTT |
| | | | CGGCGGAGGGACCAAGGTGGAGATCAAA |
| 214 | IMPI-020 | VL domain | DVVLTQSPLSLPVTLGQPASISCRSSQSLVYSDGNTYLSWFQQRPGQSPRRLIYKVSNWDSGVPDRFSGSGSGSDFT |
| | | amino acid | LKISRVEAEDVGVYYCMQGTHWPLTFGGGTKVEIK |
| | | sequence | |
| 84 | IMPI-020 | LCDR1 | QSLVYSDGNTY |
| 85 | IMPI-020 | LCDR2 | KVS |
| 86 | IMPI-020 | LCDR3 | MQGTHWPLT |
| 215 | IMPI-053 | VH domain | CAGGTGCAGCTGCAGGAGTCGGGCCCAGGACTGGTGAAACCTTCGGAGACCCTGTCCCTCACCTGCGCTGTCTCGGG |
| | | nucleic | TGGCTCCATCAGCAGAAGTACCTGGTGGAGTTGGGTCCGCCAGCCCCCAGGGAAGGGGCTGGAGTGGATTGGGGAAA |
| | | acid | TCTATCATAGTGGGAGCACCAAATACAACCCGTCCCTCAAGAGTCGAGTCACCATATCAGTAGACAAGTCCAAGAAC |
| | | sequence | CAGTTCTCCCTGAAGCTGAGCTCTGTGACCGCCGCGGACACGGCCGTGTATTACTGTGCGGGAGAGGGGTCTAACTG |
| | | | GAGCTACTGGGGCCAGGGAACCCTGGTCACCGTCTCCTCA |
| 216 | IMPI-053 | VH domain | QVQLQESGPGLVKPSETLSLTCAVSGGSISRSTWWSWVRQPPGKGLEWIGEIYHSGSTKYNPSLKSRVTISVDKSKN |
| | | amino acid | QFSLKLSSVTAADTAVYYCAGEGSNWSYWGQGTLVTVSS |
| | | sequence | |
| 79 | IMPI-053 | HCDR1 | GGSISRSTW |
| 140 | IMPI-053 | HCDR2 | IYHSGST |
| 207 | IMPI-053 | HCDR3 | AGEGSNWSY |
| 217 | IMPI-053 | VL domain | GATGTTGTGATGACTCAGTCTCCAGTCTCCCTGCCCGTCACCCTTGGACAGCCGGCCTCCATCTCCTGCAGGTCTAG |
| | | nucleic | TCAAAGCCTCATATACAGTGATGGAAACACCTACTTGAGTTGGTTTCAGCAGAGGCCAGGCCAATCTCCAAGGCGCC |
| | | acid | TAATTTATAAGGTTTCTAACTGGGACTCTGGGGTCCCAGACAGATTCAGCGGCAGTGGGTCAGGCACTGATTTCACA |
| | | sequence | CTGAAAATCAGCAGGGTGGAGGCTGAGGATATTGGGATTTATTACTGCATGCAAGGTACACACTGGCCGCTCACTTT |
| | | | CGGCGGAGGGACCAAGGTGGAGCTCAAA |
| 218 | IMPI-053 | VL domain | DVVMTQSPVSLPVTLGQPASISCRSSQSLIYSDGNTYLSWFQQRPGQSPRRLIYKVSNWDSGVPDRFSGSGSGTDFT |
| | | amino acid | LKISRVEAEDIGIYYCMQGTHWPLTFGGGTKVELK |
| | | sequence | |
| 219 | IMPI-053 | LCDR1 | QSLIYSDGNTY |
| 85 | IMPI-053 | LCDR2 | KVS |
| 86 | IMPI-053 | LCDR3 | MQGTHWPLT |
| 220 | IMPI-021 | VH domain | GAGGTGCAGCTGGTGGAGTCTGGAGGAGGCTTGATCCAGCCTGGGGGGTCCCTGAGACTCTCCTGTGCAGCCTCTGG |
| | | nucleic | GGTCACCGTCAGTAGCAACTACATGAGCTGGGTCCGCCAGGCTCCAGGGAAGGGGCTGGAGTGGGTCTCAGTTATTT |
| | | acid | ATAGCGGTGGTAGCACATTCTACGCAGACTCCGTGAAGGGCCGATTCACCATCTCCAGAGACAATTCCAAGAACACG |
| | | sequence | CTGTATCTTCAAATGAACAGCCTGAGAGCCGAGGACACGGCCGTGTATTACTGTGCGAGAGATCTCTCCTACTACGG |
| | | | TATGGACGTCTGGGGCCAAGGGACCACGGTCACCGTCTCCTCA |
| 221 | IMPI-021 | VH domain | EVQLVESGGGLIQPGGSLRLSCAASGVTVSSNYMSWVRQAPGKGLEWVSVIYSGGSTFYADSVKGRFTISRDNSKNT |
| | | amino acid | LYLQMNSLRAEDTAVYYCARDLSYYGMDVWGQGTTVTVSS |
| | | sequence | |
| 3 | IMPI-021 | HCDR1 | GVTVSSNY |
| 14 | IMPI-021 | HCDR2 | IYSGGST |
| 222 | IMPI-021 | HCDR3 | ARDLSYYGMDV |
| 223 | IMPI-021 | VL domain | GACATCCAGTTGACCCAGTCTCCATCCTTCCTGTCTGCATCTGTAGGAGACAGAGTCACCATCACTTGCTGGGCCAG |
| | | nucleic | TCAGGGCATTAGCAGTTATTTAGCCTGGTATCAGCAAAAACCAGGGAAAGCCCCTAAGCTCCTGATCTATGCTGCAT |
| | | acid | CCACTTTGCAAAGTGGGGTCCCATCAAAATTCAGCGGCAGTGGATCTGGGACAGAATTCACTCTCACAATCAGCAGC |
| | | sequence | CTGCAGCCTGAAGATTTTGCAACTTATTACTGTCAACAGCTTAATAGTTACCCGTGCAGTTTTGGCCAGGGGACCAA |
| | | | GCTGGAGATCAAA |
| 224 | IMPI-021 | VL domain | DIQLTQSPSFLSASVGDRVTITCWASQGISSYLAWYQQKPGKAPKLLIYAASTLQSGVPSKFSGSGSGTEFTLTISS |
| | | amino acid | LQPEDFATYYCQQLNSYPCSFGQGTKLEIK |
| | | sequence | |
| 8 | IMPI-021 | LCDR1 | QGISSY |
| 18 | IMPI-021 | LCDR2 | AAS |
| 225 | IMPI-021 | LCDR3 | QQLNSYPCS |
| 226 | IMPI-032 | VH domain | CAGGTGCAGCTGCAGGAGTCGGGCCCAGGACTGGTGAAGCCTTCGGGGACCCTGTCCCTCACCTGCGCTGTCTCTGG |
| | | nucleic | TGGCTCCATCAGCAGTAATAAGTGGTGGAGTTGGGTCCGCCAGCCCCCAGGGAAGGGTCTGGAGTGGATTGGGGAAA |
| | | acid | TCGATCATAGTGGGAGCACCATGTACAACCCGTCCCTCAAGAGCCGAGTCACCATATCAGTAGACAAGTCCAAGAAC |
| | | sequence | CAGTTCTCCCTGAAGCTGAACTCTGTGACCGCCGCGGACACGGCCGTGTATTACTGTACGGGAGAGGGGTATAACTG |
| | | | GAACTACTGGGGCCAGGGGACCCGGGTCACCGTCTCCTCA |
| 227 | IMPI-032 | VH domain | QVQLQESGPGLVKPSGTLSLTCAVSGGSISSNKWWSWVRQPPGKGLEWIGEIDHSGSTMYNPSLKSRVTISVDKSKN |
| | | amino acid | QFSLKLNSVTAADTAVYYCTGEGYNWNYWGQGTRVTVSS |
| | | sequence | |
| 228 | IMPI-032 | HCDR1 | GGSISSNKW |
| 132 | IMPI-032 | HCDR2 | IDHSGST |
| 133 | IMPI-032 | HCDR3 | TGEGYNWNY |
| 229 | IMPI-032 | VL domain | GATGTTGTGATGACTCAGTCTCCACTCTCCCTGCCCGTCACCCTTGGACAGACGGCCTCCATCTCCTGCAGGTCTAG |
| | | nucleic | TCAAAGCCTCCTATACACTGATGGAAATACCTACTTGAGTTGGTTTCAGCAGAGGCCAGGCCAATCTCCAAGGCGCC |
| | | acid | TAATTTATAAGGTTTCTAAGTGGGAATCTGGGGTCCCAGACAGATTCAGCGGCAGTGGGTCAGGCACTGATTTCACA |
| | | sequence | CTGAAAATCAGCAGGGTGGAGGCTGAAGATGTTGGGATTTATTACTGCATGCAAGGTACACACTGGCCGCTCACTTT |
| | | | CGGCGGAGGGACCAAGGTGGAGATCAAA |
| 230 | IMPI-032 | VL domain | DVVMTQSPLSLPVTLGQTASISCRSSQSLLYTDGNTYLSWFQQRPGQSPRRLIYKVSKWESGVPDRFSGSGSGTDFT |
| | | amino acid | LKISRVEAEDVGIYYCMQGTHWPLTFGGGTKVEIK |
| | | sequence | |
| 136 | IMPI-032 | LCDR1 | QSLLYTDGNTY |
| 85 | IMPI-032 | LCDR2 | KVS |
| 86 | IMPI-032 | LCDR3 | MQGTHWPLT |
| 226 | IMPI-001 | VH domain | CAGGTGCAGCTGCAGGAGTCGGGCCCAGGACTGGTGAAGCCTTCGGGGACCCTGTCCCTCACCTGCGCTGTCTCTGG |
| | | nucleic | TGGCTCCATCAGCAGTAATAAGTGGTGGAGTTGGGTCCGCCAGCCCCCAGGGAAGGGTCTGGAGTGGATTGGGGAAA |
| | | acid | TCGATCATAGTGGGAGCACCATGTACAACCCGTCCCTCAAGAGCCGAGTCACCATATCAGTAGACAAGTCCAAGAAC |
| | | sequence | CAGTTCTCCCTGAAGCTGAACTCTGTGACCGCCGCGGACACGGCCGTGTATTACTGTACGGGAGAGGGGTATAACTG |
| | | | GAACTACTGGGGCCAGGGGACCCGGGTCACCGTCTCCTCA |
| 227 | IMPI-001 | VH domain | QVQLQESGPGLVKPSGTLSLTCAVSGGSISSNKWWSWVRQPPGKGLEWIGEIDHSGSTMYNPSLKSRVTISVDKSKN |
| | | amino acid | QFSLKLNSVTAADTAVYYCTGEGYNWNYWGQGTRVTVSS |
| | | sequence | |
| 228 | IMPI-001 | HCDR1 | GGSISSNKW |
| 132 | IMPI-001 | HCDR2 | IDHSGST |
| 133 | IMPI-001 | HCDR3 | TGEGYNWNY |
| 231 | IMPI-001 | VL domain | GATGTTGTGCTGACTCAGTCTCCACTCTCCCTGCCCGTCACCCTTGGACAGACGGCCTCCATCTCCTGCAGGTCTAG |
| | | nucleic | TCAAAGCCTCGTATACACTGATGGAAATACCTACTTGAGTTGGTTTCAGCAGAGGCCAGGCCAATCTCCAAGGCGCC |
| | | acid | TAATTTATAAGGTTTCTAAGTGGGAATCTGGGGTCCCAGACAGATTCAGCGGCAGTGGGTCAGGCACTGATTTCACC |
| | | sequence | CTGAAAATCAGCAGGGTGGAGGCTGAAGATGTTGGGATTTATTACTGCATGCAAGGTACACACTGGCCGCTCACTTT |
| | | | CGGCGGAGGGACCATGGTGGAGATCACA |
| 232 | IMPI-001 | VL domain | DVVLTQSPLSLPVTLGQTASISCRSSQSLVYTDGNTYLSWFQQRPGQSPRRLIYKVSKWESGVPDRFSGSGSGTDFT |
| | | amino acid | LKISRVEAEDVGIYYCMQGTHWPLTFGGGTMVEIT |
| | | sequence | |
| 144 | IMPI-001 | LCDR1 | QSLVYTDGNTY |
| 85 | IMPI-001 | LCDR2 | KVS |
| 86 | IMPI-001 | LCDR3 | MQGTHWPLT |
| 233 | IMPI-041 | VH domain | CAGGTGCAGCTGGTGCAGTCTGGGGCTGAGGTGAAGAAGCCTGGGGCCTCAGTGAAGGTCTCCTGCAAGGCTTCTGG |
| | | nucleic | ATACACCTTCACCAGTTATGATATCAACTGGGTGCGACAGGCCACTGGACAAGGGCTTGAGTGGATGGGATGGATGA |
| | | acid | ACCCTAACAGTGGTAACACAGGCTATGCACAGAAGTTCCAGGGCAGAGTCACCATGACCAGAAACACCTCCATAAGC |
| | | sequence | ACAGCCTACATGGAGCTGAGCAGCCTGAGATCTGAGGACACGGCCGTGTATTTCTGTGCGAGAGGCGGGCGATATTG |
| | | | TAGTGATGTTAGCTGCTACTCCGGAGAGCCTTTTGACTACTGGGGCCAGGGAACCCTGGTCACCGTCTCCTCA |
| 234 | IMPI-041 | VH domain | QVQLVQSGAEVKKPGASVKVSCKASGYTFTSYDINWVRQATGQGLEWMGWMNPNSGNTGYAQKFQGRVTMTRNTSIS |
| | | amino acid | TAYMELSSLRSEDTAVYFCARGGRYCSDVSCYSGEPFDYWGQGTLVTVSS |
| | | sequence | |
| 121 | IMPI-041 | HCDR1 | GYTFTSYD |
| 235 | IMPI-041 | HCDR2 | MNPNSGNT |
| 236 | IMPI-041 | HCDR3 | ARGGRYCSDVSCYSGEPFDY |
| 237 | IMPI-041 | VL domain | GAAATTGTGCTGACTCAGTCTCCAGACTTTCAGTCTGTGACTCCAAAGGAGAAAGTCACCATCACCTGCCGGGCCAG |
| | | nucleic | TCAGAGCATTGGTAGTAGCTTACACTGGTTCCAGCAGAAACCAGATCAGTCTCCAAAGCTCCTCATCAAGTATACTT |
| | | acid | CCCAGTCCATCTCAGGGGTCCCCTCGAGGTTCAGTGGCAGTGGATCTGGGACAGATTTCACCCTCACCATCAATAGC |
| | | sequence | CTGGAAGCTGAAGATGCTGCAGCGTATTATTGTCATCAGAGTAGTAGTTTACCTCACACTTTCGGCCCTGGGACCAA |
| | | | AGTGGAGATCAAA |
| 238 | IMPI-041 | VL domain | EIVLTQSPDFQSVTPKEKVTITCRASQSIGSSLHWFQQKPDQSPKLLIKYTSQSISGVPSRFSGSGSGTDFTLTINS |
| | | amino acid | LEAEDAAAYYCHQSSSLPHTFGPGTKVEIK |
| | | sequence | |
| 126 | IMPI-041 | LCDR1 | QSIGSS |
| 239 | IMPI-041 | LCDR2 | YTS |
| 128 | IMPI-041 | LCDR3 | HQSSSLPHT |
| 240 | IMPI-029 | VH domain | GAGGTGCAGCTGGTGGAGTCTGGAGGAGGCTTGATCCAGCCTGGGGGGTCCCTGAGACTCTCCTGTGCAGCCTCTGG |
| | | nucleic | GGTCACCGTCAGTAGCAACTATATGAACTGGGTCCGCCAGGCTCCAGGGAAGGGGCTGGAGTGGGTCTCAGTTATTT |
| | | acid | ATAGCGGTGGTAGCACATACTACGCAGACTCCGTGAAGGGCCGATTCACCATCTCCAGAGACAATTCCAAGAACACG |
| | | sequence | CTGTTTCTTCAAATGAACAGCCTGAGAGCCGAGGACTCGGCCGTGTATTACTGTGCGAGAGATTTGGGACCCTACGG |
| | | | TGTGGACGTCTGGGGCCAAGGGACCACGGTCACCGTCTCCTCA |
| 241 | IMPI-029 | VH domain | EVQLVESGGGLIQPGGSLRLSCAASGVTVSSNYMNWVRQAPGKGLEWVSVIYSGGSTYYADSVKGRFTISRDNSKNT |
| | | amino acid | LFLQMNSLRAEDSAVYYCARDLGPYGVDVWGQGTTVTVSS |
| | | sequence | |
| 3 | IMPI-029 | HCDR1 | GVTVSSNY |
| 14 | IMPI-029 | HCDR2 | IYSGGST |
| 106 | IMPI-029 | HCDR3 | ARDLGPYGVDV |
| 107 | IMPI-029 | VL domain | GACATCCAGTTGACCCAGTCTCCATCCTTCCTGTCTGCATCTGTAGGAGACAGAGTCACCATCACTTGCTGGGCCAG |
| | | nucleic | TCAGGGCATTAGCAGTTATTTAGCCTGGTATCAGCAAAAACCAGGGAAAGCCCCTAAGCTCCTGATCTATGCTGCAT |
| | | acid | CCACTTTGCAAAGTGGGGTCCCATCAAGGTTCAGCGGCAGTGGATCTGGGACAGAATTCACTCTCACAATCAGCAGC |
| | | sequence | CTGCAGCCTGAAGATTTTGCAACTTATTACTGTCAAGAGCTTAATAGTTACCCTCTCACCTTCGGCCAAGGGACACG |
| | | | ACTGGAGATTAAA |
| 108 | IMPI-029 | VL domain | DIQLTQSPSFLSASVGDRVTITCWASQGISSYLAWYQQKPGKAPKLLIYAASTLQSGVPSRFSGSGSGTEFTLTISS |
| | | amino acid | LQPEDFATYYCQELNSYPLTFGQGTRLEIK |
| | | sequence | |
| 8 | IMPI-029 | LCDR1 | QGISSY |
| 18 | IMPI-029 | LCDR2 | AAS |
| 109 | IMPI-029 | LCDR3 | QELNSYPLT |
| 242 | IMPI-009 | VH domain | GAGGTGCAGCTGGTGCAGTCTGGAGTAGAGGTGAAAAAGCCCGGGGAGTCTCTGAAGATCTCCTGTAAGGGTTCTGG |
| | | nucleic | ATACAGCTTTACCAGCTACTGGATCGGCTGGGTGCGCCAGATGCCCGGGAAAGGCCTGGAGTGGATGGGGATCATCT |
| | | acid | ATCCTGGTGACTCTGATACCAGATACAGCCCGTCCTTCCAAGGCCAGGTCACCATCTCAGCCGACAAGTCCATCAGT |
| | | sequence | ACCGCCTACCTGCAGTGGAGCAGCCTGAAGGCCTCGGACACCGCCATGTATTACTGTGCGAGGTCGTATAACTGGAA |
| | | | CTACTTTGACTACTGGGGCCAGGGAACCCTGGTCACCGTCTCCTCA |
| 243 | IMPI-009 | VH domain | EVQLVQSGVEVKKPGESLKISCKGSGYSFTSYWIGWVRQMPGKGLEWMGIIYPGDSDTRYSPSFQGQVTISADKSIS |
| | | amino acid | TAYLQWSSLKASDTAMYYCARSYNWNYFDYWGQGTLVTVSS |
| | | sequence | |
| 32 | IMPI-009 | HCDR1 | GYSFTSYW |
| 33 | IMPI-009 | HCDR2 | IYPGDSDT |
| 34 | IMPI-009 | HCDR3 | ARSYNWNYFDY |
| 244 | IMPI-009 | VL domain | GCCATCCAGTTGACCCAGTCTCCATCCTCCCTGTCTGCATCTGTAGGAGACAGAGTCACCATCACTTGCCGGGCAAG |
| | | nucleic | TCAGGGCATTAGCAGTGGTTTAGCCTGGTATCAGCAGAAACCAGGGAAAGCTCCTGAGCTCCTGATCTATGATGCCT |
| | | acid | CCAGTTTGGAAAGTGGGGTCCCATCAAGGTTCAGCGGCAGTGGATCTGGGACAGATTTCACTCTCACCATCAGCAGC |
| | | sequence | CTGCAGCCTGAAGATTTTGCAACTTATTACTGTCAACAGTTTTATAGTTACCCTTGGACGTTCGGCCAAGGGACCAA |
| | | | GGTGGAAATCAAA |
| 245 | IMPI-009 | VL domain | AIQLTQSPSSLSASVGDRVTITCRASQGISSGLAWYQQKPGKAPELLIYDASSLESGVPSRFSGSGSGTDFTLTISS |
| | | amino acid | LQPEDFATYYCQQFYSYPWTFGQGTKVEIK |
| | | sequence | |
| 92 | IMPI-009 | LCDR1 | QGISSG |
| 28 | IMPI-009 | LCDR2 | DAS |
| 246 | IMPI-009 | LCDR3 | QQFYSYPWT |
| 247 | IMPI-006 | VH domain | CAGCTGCAGCTGCAGGAGTCGGGCCCAGGACTGGTGAAGCCTTCGGAGACCCTGTCCCTCACCTGCACTGTCTCTGG |
| | | nucleic | TGGCTCCATCAGTAATAGTAATTACTACTGGGGCTGGATCCGCCAGCCCCCAGGGAAGGGGCTGGAGTGGATTGGGA |
| | | acid | GTATCTATTATAGTGGGAGAACCTACTACAACCCGTCCCTCAAGAGTCGAGTCACCATATCCGTAGACACGTCCAAG |
| | | sequence | AACCAGTTCTCCCTGAAGTTGAGCTCTGTGACCGCCGCAGACACGGCTGTGTATTACTGTGCGAGAATGGGATTACT |
| | | | ATGGTTCGGGGAGTTCTACGGTATGGACGTCTGGGGCCAAGGGACCACGGTCACCGTCTCCTCA |
| 248 | IMPI-006 | VH domain | QLQLQESGPGLVKPSETLSLTCTVSGGSISNSNYYWGWIRQPPGKGLEWIGSIYYSGRTYYNPSLKSRVTISVDTSK |
| | | amino acid | NQFSLKLSSVTAADTAVYYCARMGLLWFGEFYGMDVWGQGTTVTVSS |
| | | sequence | |
| 249 | IMPI-006 | HCDR1 | GGSISNSNYY |
| 250 | IMPI-006 | HCDR2 | IYYSGRT |
| 251 | IMPI-006 | HCDR3 | ARMGLLWFGEFYGMDV |
| 252 | IMPI-006 | VL domain | GACATCCAGATGACCCAGTCTCCATCCTCACTGTCTGCATCTGTAGGAGACAGAGTCACCATCACTTGTCGGGCGAG |
| | | nucleic | TCAGGGTATTAGCAGCTGGTTAGCCTGGTATCAGCAGAAACCAGAGAAAGCCCCTAAGTCCCTGATCTATGATGCAT |
| | | acid | CCAGTTTACAAAGAGGGGTCCCATCAAGGTTCAGCGGCAGTGGATCTGGGACAGATTTCACTCTCACCATCAGCAGC |
| | | sequence | CTGCAGCCTGAAGATTTTGCATCTTATTACTGCCAACAGTATAATAGTTACCCGCTCACTTTCGGCGGAGGGACCAG |
| | | | GGTGGAGATCAAA |
| 253 | IMPI-006 | VL domain | DIQMTQSPSSLSASVGDRVTITCRASQGISSWLAWYQQKPEKAPKSLIYDASSLQRGVPSRFSGSGSGTDFTLTISS |
| | | amino acid | LQPEDFASYYCQQYNSYPLTFGGGTRVEIK |
| | | sequence | |
| 254 | IMPI-006 | LCDR1 | QGISSW |
| 28 | IMPI-006 | LCDR2 | DAS |
| 255 | IMPI-006 | LCDR3 | QQYNSYPLT |
| 256 | IMPI-054 | VH domain | GAGGTGCAACTGGTGGAGTCTGGAGGAGGCTTGATCCAGCCTGGGGGGTCCCTGAGACTCTCCTGTGCAGCCTCTGG |
| | | nucleic | GCTCACCGTCAGTAGCAACTACATGAGCTGGGTCCGCCAGGCTCCAGGGAAGGGGCTGGAGTGGGTCTCAATTATTT |
| | | acid | ATAGCGGTGGTAGCACATACTACGCAGACTCCGTGAAGGGCCGATTCACCATCTCCAGAGACAATTCCAAGAACACG |
| | | sequence | CTGTATCTTCAAATGAACAGCCTGAGAGCCGAGGACACGGCCGTGTATTACTGTGCGAGGCTAGGGGGGTACTACTA |
| | | | CTACGGTATGGACGTCTGGGGCCAAGGGACCACGGTCACCGTCTCCTCA |
| 257 | IMPI-054 | VH domain | EVQLVESGGGLIQPGGSLRLSCAASGLTVSSNYMSWVRQAPGKGLEWVSIIYSGGSTYYADSVKGRFTISRDNSKNT |
| | | amino acid | LYLQMNSLRAEDTAVYYCARLGGYYYYGMDVWGQGTTVTVSS |
| | | sequence | |
| 172 | IMPI-054 | HCDR1 | GLTVSSNY |
| 14 | IMPI-054 | HCDR2 | IYSGGST |
| 258 | IMPI-054 | HCDR3 | ARLGGYYYYGMDV |
| 259 | IMPI-054 | VL domain | GACATCCAGATGACCCAGTCTCCATCCTCCCTGTCTGCATCTGTAGGAGAAAGAGTCACCATCACTTGCCAGGCGAG |
| | | nucleic | TCAGGACATTAGAAACTATTTAAATTGGTATCAGAAGAAACCAGGGAAAGCCCCTAAGCTCCTGATCTACGATTCAT |
| | | acid | CCAATTTGGAAACAGGGGTCCCATCAAGGTTCAGTGGAAGTGGATCTGGGACAGATTTTACTTTCACCATCAGCAGC |
| | | sequence | CTGCAGCCTGAAGATATTGCAACATATTACTGTCAACAGTATGATAATCTCCCGATCACCTTCGGCCAAGGGACACG |
| | | | ACTGGAGATTAAA |
| 260 | IMPI-054 | VL domain | DIQMTQSPSSLSASVGERVTITCQASQDIRNYLNWYQKKPGKAPKLLIYDSSNLETGVPSRFSGSGSGTDFTFTISS |
| | | amino acid | LQPEDIATYYCQQYDNLPITFGQGTRLEIK |
| | | sequence | |
| 261 | IMPI-054 | LCDR1 | QDIRNY |
| 262 | IMPI-054 | LCDR2 | DSS |
| 263 | IMPI-054 | LCDR3 | QQYDNLPIT |
| 264 | IMPI-044 | VH domain | GAGGTGCAGCTGGTGCAGTCTGGAGCAGAGGTGAAAAAGCCCGGGGAGTCTCTGAAGATCTCCTGTAAGGGTTCTGG |
| | | nucleic | ATACAGCTTTACCAGCTACTGGATCGGCTGGGTGCGCCAGATGCCCGGGAAAGGCCTGGAGTGGATGGGGATCATCT |
| | | acid | ATCCTGGTGACTCTGATACCAGATACAGCCCGTCCTTCCAAGGCCAGGTCACCATCTCAGCCGACAAGTCCATCAGC |
| | | sequence | ACCGCCTACCTGCAGTGGAGCAGCCTGAAGGCCTCGGACACCGCCATGTATTACTGTGCGAGGTCGTATAACTGGAA |
| | | | CTACTTTGACTACTGGGGCCAGGGAACCCTGGTCACCGTCTCCTCA |
| 265 | IMPI-044 | VH domain | EVQLVQSGAEVKKPGESLKISCKGSGYSFTSYWIGWVRQMPGKGLEWMGIIYPGDSDTRYSPSFQGQVTISADKSIS |
| | | amino acid | TAYLQWSSLKASDTAMYYCARSYNWNYFDYWGQGTLVTVSS |
| | | sequence | |
| 32 | IMPI-044 | HCDR1 | GYSFTSYW |
| 33 | IMPI-044 | HCDR2 | IYPGDSDT |
| 34 | IMPI-044 | HCDR3 | ARSYNWNYFDY |
| 266 | IMPI-044 | VL domain | GCCATCCAGTTGACCCAGTCTCCATCCTCCCTGTCTGCATCTGTTGGAGACAGAGTCACCATCACTTGCCGGGCAAG |
| | | nucleic | TCAGGGCATTAGTAGTGGTTTAGCCTGGTATCAGCAGAAACCAGGGAAAGCTCCTAAGCTCCTGATCTATGATGTCT |
| | | acid | CCAGTTTGGAAAGTGGGGTCCCATCAAGGTTCAGCGGCAGTGGATCTGGGACAGATTTCACTCTCACCATCAGCAGC |
| | | sequence | CTGCAGCCTGAAGATTTTGCAACTTATTACTGTCAACAGTTTCTTAGTTACCCTTGGACGTTCGGCCAAGGGACCAA |
| | | | GGTGGAAATCAAA |
| 267 | IMPI-044 | VL domain | AIQLTQSPSSLSASVGDRVTITCRASQGISSGLAWYQQKPGKAPKLLIYDVSSLESGVPSRFSGSGSGTDFTLTISS |
| | | amino acid | LQPEDFATYYCQQFLSYPWTFGQGTKVEIK |
| | | sequence | |
| 92 | IMPI-044 | LCDR1 | QGISSG |
| 203 | IMPI-044 | LCDR2 | DVS |
| 204 | IMPI-044 | LCDR3 | QQFLSYPWT |
| 268 | IMPI-002 | VH domain | GAGGTGCAGCTGGTGGAGTCTGGAGGAGGCTTGATCCAGCCTGGGGGGTCCCTGAGACTCTCCTGTGCAGCCTCTGG |
| | | nucleic | GGTCACCGTCAGTAGCAACTACATGAGCTGGGTCCGCCAGGCTCCAGGGAAGGGGCTGGAGGGGGTCTCAGTTATTT |
| | | acid | ATAGTGGTGGTAGCACATACTACGCAGACTCCGTGAAGGGCCGATTCACCATCTCCAGAGACAATTCCAAGAACACG |
| | | sequence | CTGTATCTTCAAATGAACAGCCTGAGAGCCGAGGACACGGCCGTGTATTACTGTGCGAGAGATACGGTGGTCTACGG |
| | | | TATGGACGTCTGGGGCCAAGGGACCACGGTCACCGTCTCCTCA |
| 269 | IMPI-002 | VH domain | EVQLVESGGGLIQPGGSLRLSCAASGVTVSSNYMSWVRQAPGKGLEGVSVIYSGGSTYYADSVKGRFTISRDNSKNT |
| | | amino acid | LYLQMNSLRAEDTAVYYCARDTVVYGMDVWGQGTTVTVSS |
| | | sequence | |
| 3 | IMPI-002 | HCDR1 | GVTVSSNY |
| 14 | IMPI-002 | HCDR2 | IYSGGST |
| 5 | IMPI-002 | HCDR3 | ARDTVVYGMDV |
| 270 | IMPI-002 | VL domain | GACATCCAGTTGACCCAGTCTCCATCCTTCCTGTCTGCATCTGTAGGAGACAGAGTCACCATCACTTGCTGGGCCAG |
| | | nucleic | TCAGGGCATTAGCAGTTATTTAGCCTGGTATCAGCAAAAACCAGGGAAAGCCCCTAAGCTCCTGATCTATGCTGCAT |
| | | acid | CCACTTTGCAAAGTGGGGTCCCATCAAGGTTCAGCGGCAGTGGATCTGGGACAGAATTCACTCTCACAATCAGCAGC |
| | | sequence | CTGCAGCCTGAAGATTTTGCAACTTATTACTGTCAACAGCTTAATAGTTACCCGCTCACTTTCGGCGGAGGGACCAA |
| | | | GGTGGAGATCAAA |
| 271 | IMPI-002 | VL domain | DIQLTQSPSFLSASVGDRVTITCWASQGISSYLAWYQQKPGKAPKLLIYAASTLQSGVPSRFSGSGSGTEFTLTISS |
| | | amino acid | LQPEDFATYYCQQLNSYPLTFGGGTKVEIK |
| | | sequence | |
| 8 | IMPI-002 | LCDR1 | QGISSY |
| 18 | IMPI-002 | LCDR2 | AAS |
| 10 | IMPI-002 | LCDR3 | QQLNSYPLT |
| 272 | IMPI-027 | VH domain | CAGGTGCAGCTGCAGGAGTCGGGCCCAGGACTGGTGAAGCCTTCACAGACCCTGTCCCTCACCTGCACTGTCTCTGG |
| | | nucleic | TGGCTCCATCAGCAGTTATGGTTACTACTGGAACTGGATCCGCCAGCACCCAGGGAAGGGCCTGGAGTGGATTGGGT |
| | | acid | ACATCTATTACAGTGGGAGCACCTACTACAACCCGTCCCTCAAGAGTCGAGTTACCATATCAGTAGACACGTCTAAG |
| | | sequence | AACCAGCTCTCCCTGAGGCTGAACTCTGTCAGTGCCGCGGACACGGCCGTGTATTACTGTGCGAGAGACTTCGGTGG |
| | | | TAACTCGAACTACTTTGACTACTGGGGCCAGGGAACCCTGGTCACCGTCTCCTCA |
| 273 | IMPI-027 | VH domain | QVQLQESGPGLVKPSQTLSLTCTVSGGSISSYGYYWNWIRQHPGKGLEWIGYIYYSGSTYYNPSLKSRVTISVDTSK |
| | | amino acid | NQLSLRLNSVSAADTAVYYCARDFGGNSNYFDYWGQGTLVTVSS |
| | | sequence | |
| 41 | IMPI-027 | HCDR1 | GGSISSYGYY |
| 42 | IMPI-027 | HCDR2 | IYYSGST |
| 43 | IMPI-027 | HCDR3 | ARDFGGNSNYFDY |
| 274 | IMPI-027 | VL domain | GCCATCCAGTTGACCCAGTCTCCATCCTCCCTGTCTGCATCTGTAGGAGACAGAGTCACCATCACTTGCCGGACAAG |
| | | nucleic | TCAGGGCATTAGCAGTGCTTTAGCCTGGTATCAACAGAAACCAGGGAAAGCTCCTAAACTCCTGATCTATGATGCCT |
| | | acid | CCAGTTTGGAAAGTGGGGTCCCATCAAGGTTCAGCGGCAGTGGATCTGGGACAGATTTCACTCTCACCATCAGCAGC |
| | | sequence | CTGCAGCCTGAAGATTTTGCAACTTATTACTGTCAACAGTTTAATAGTTACCCTCTCACTTTCGGCGGAGGGACCAA |
| | | | GGTGGAGATCAAA |
| 275 | IMPI-027 | VL domain | AIQLTQSPSSLSASVGDRVTITCRTSQGISSALAWYQQKPGKAPKLLIYDASSLESGVPSRFSGSGSGTDFTLTISS |
| | | amino acid | LQPEDFATYYCQQFNSYPLTFGGGTKVEIK |
| | | sequence | |
| 37 | IMPI-027 | LCDR1 | QGISSA |
| 28 | IMPI-027 | LCDR2 | DAS |
| 47 | IMPI-027 | LCDR3 | QQFNSYPLT |
| 264 | IMPI-011 | VH domain | GAGGTGCAGCTGGTGCAGTCTGGAGCAGAGGTGAAAAAGCCCGGGGAGTCTCTGAAGATCTCCTGTAAGGGTTCTGG |
| | | nucleic | ATACAGCTTTACCAGCTACTGGATCGGCTGGGTGCGCCAGATGCCCGGGAAAGGCCTGGAGTGGATGGGGATCATCT |
| | | acid | ATCCTGGTGACTCTGATACCAGATACAGCCCGTCCTTCCAAGGCCAGGTCACCATCTCAGCCGACAAGTCCATCAGC |
| | | sequence | ACCGCCTACCTGCAGTGGAGCAGCCTGAAGGCCTCGGACACCGCCATGTATTACTGTGCGAGGTCGTATAACTGGAA |
| | | | CTACTTTGACTACTGGGGCCAGGGAACCCTGGTCACCGTCTCCTCA |
| 265 | IMPI-011 | VH domain | EVQLVQSGAEVKKPGESLKISCKGSGYSFTSYWIGWVRQMPGKGLEWMGIIYPGDSDTRYSPSFQGQVTISADKSIS |
| | | amino acid | TAYLQWSSLKASDTAMYYCARSYNWNYFDYWGQGTLVTVSS |
| | | sequence | |
| 32 | IMPI-011 | HCDR1 | GYSFTSYW |
| 33 | IMPI-011 | HCDR2 | IYPGDSDT |
| 34 | IMPI-011 | HCDR3 | ARSYNWNYFDY |
| 276 | IMPI-011 | VL domain | GCCATCCAGTTGACCCAGTCTCCATCCTCCCTGTCTGCATCTGTAGGAGACAGAGTCACCATCACTTGCCGGGCAAG |
| | | nucleic | TCAGGGCATTAGCAGTGGTTTAGCCTGGTATCAGCAGAAACCAGGGAAAGCTCCTAAACTCCTGATCTATGATGCCT |
| | | acid | CCAGTTTGGAAAGTGGGGTCCCATCAAGGTTCAGCGGCAGTGGATCTGGGACAGATTTCACTCTCACCATCAGCAGC |
| | | sequence | CTGCAGCCTGAAGATTTTGCAATTTATTACTGTCAACAGTTTAATAGTTATCCTTGGACGTTCGGCCAAGGGACCAA |
| | | | GGTGGAAATCAAA |
| 277 | IMPI-011 | VL domain | AIQLTQSPSSLSASVGDRVTITCRASQGISSGLAWYQQKPGKAPKLLIYDASSLESGVPSRFSGSGSGTDFTLTISS |
| | | amino acid | LQPEDFAIYYCQQFNSYPWTFGQGTKVEIK |
| | | sequence | |
| 92 | IMPI-011 | LCDR1 | QGISSG |
| 28 | IMPI-011 | LCDR2 | DAS |
| 76 | IMPI-011 | LCDR3 | QQFNSYPWT |
| 278 | IMPI-033 | VH domain | CAGGTGCAGCTGCAGGAGTCGGGCCCAGGACTGGTGAAGCCTTCACAGACCCTGTCCCTCACCTGCACTGTCTCTGG |
| | | nucleic | TGGCTCCATCAGCAGTTATGGTTACTACTGGAGCTGGATCCGCCAGCACCCAGGGAAGGGCCTGGAGTGGATTGGAT |
| | | acid | ACATCTATTACAGTGGGAGCACCTACTACAACCCGTCCCTCAAGAGTCGAGTTACCATGTCAGTTGACACGTCTAAG |
| | | sequence | AACCAGCTCTCCCTGAGGCTGAACTCTGTCAGTGCCGCGGACACGGCCGTGTATTACTGTGCGAGAGACTTCGGTGG |
| | | | TAACTCGAACTACTTTGACTACTGGGGCCAGGGAACCCTGGTCACCGTCTCCTCA |
| 279 | IMPI-033 | VH domain | QVQLQESGPGLVKPSQTLSLTCTVSGGSISSYGYYWSWIRQHPGKGLEWIGYIYYSGSTYYNPSLKSRVTMSVDTSK |
| | | amino acid | NQLSLRLNSVSAADTAVYYCARDFGGNSNYFDYWGQGTLVTVSS |
| | | sequence | |
| 41 | IMPI-033 | HCDR1 | GGSISSYGYY |
| 42 | IMPI-033 | HCDR2 | IYYSGST |
| 43 | IMPI-033 | HCDR3 | ARDFGGNSNYFDY |
| 280 | IMPI-033 | VL domain | GCCATCCAGTTGACCCAGTCTCCATCCTCCCTGTCTGCATCTGTAGGAGACAGAGTCACCATCACTTGCCGGGCAAG |
| | | nucleic | TCAGGGCATTAGCAGTGCTTTAGCCTGGTATCAGCAGAAACCAGGGAAAGCTCCTAACCTCCTGATCTATGATGCCT |
| | | acid | CCAGTTTGGAAAGTGGGGTCCCATCAAGGTTCAGCGGCAGTGGATCTGGGACAGATTTCACTCTCACCATCAGCAGC |
| | | sequence | CTGCAGCCTGAAGATTTTGCAACTTATTACTGTCAACAGTTTAATAGTTCCCCTCTCACTTTCGGCGGAGGGACCAA |
| | | | GGTGGAGATCAAA |
| 281 | IMPI-033 | VL domain | AIQLTQSPSSLSASVGDRVTITCRASQGISSALAWYQQKPGKAPNLLIYDASSLESGVPSRFSGSGSGTDFTLTISS |
| | | amino acid | LQPEDFATYYCQQFNSSPLTFGGGTKVEIK |
| | | sequence | |
| 37 | IMPI-033 | LCDR1 | QGISSA |
| 28 | IMPI-033 | LCDR2 | DAS |
| 282 | IMPI-033 | LCDR3 | QQFNSSPLT |
| 283 | IMPI-055 | VH domain | CAGGTGCAGCTGGTGCAGTCTGGGGCTGAGGTGAAGAAGCCTGGGGCCTCAGTGAAGGTCTCCTGCAAGGCTTCTGG |
| | | nucleic | ATACACCTTCACCAGTTATGATATCAACTGGGTGCGACAGGCCACTGGACAAGGGCTTGAGTGGATGGGATGGATGA |
| | | acid | ACCCTAACAGTGGTAACACAGGCTATGCACAGAAGTTCCAGGGCAGAGTCACCATGACCAGAGACACCTCCATAAAC |
| | | sequence | ACAGCCTACATGGAGCTGAGCAGCCTGAGATCTGAGGACACGGCCGTGTATTTCTGTGCGAGAGGCGGGCGATATTG |
| | | | TAGTGATGTTACCTGCTACTCCGGAGAGCCTTTTGACTACTGGGGCCAGGGAACCCTGGTCACCGTCTCCTCA |
| 284 | IMPI-055 | VH domain | QVQLVQSGAEVKKPGASVKVSCKASGYTFTSYDINWVRQATGQGLEWMGWMNPNSGNTGYAQKFQGRVTMTRDTSIN |
| | | amino acid | TAYMELSSLRSEDTAVYFCARGGRYCSDVTCYSGEPFDYWGQGTLVTVSS |
| | | sequence | |
| 121 | IMPI-055 | HCDR1 | GYTFTSYD |
| 235 | IMPI-055 | HCDR2 | MNPNSGNT |
| 285 | IMPI-055 | HCDR3 | ARGGRYCSDVTCYSGEPFDY |
| 286 | IMPI-055 | VL domain | GAAATTATGCTGACTCAGTCTCCAGACTTTCAGTCTGTGACTCCAAAGGAGAAAGTCACCATCACCTGCCGGGCCAG |
| | | nucleic | TCAGAGCATTGGTAGTAGCTTACACTGGTACCAGCAGAAACCAGATCAGTCTCCAAAACTCCTCATCAAGTTTGCTT |
| | | acid | CCCAGTCCATCTCAGGGGTCCCCTCGAGGTTCAGTGGCAGTGGATCTGGGACAGATTTCACCCTCACCATCAATAGC |
| | | sequence | CTGGAAGCTGAAGATGCTGCAGCGTATTATTGTCATCAGAGTAGTAGTTTACCTCACACTTTCGGCCCTGGGACCAA |
| | | | AGTGGAGATCAAA |
| 287 | IMPI-055 | VL domain | EIMLTQSPDFQSVTPKEKVTITCRASQSIGSSLHWYQQKPDQSPKLLIKFASQSISGVPSRFSGSGSGTDFTLTINS |
| | | amino acid | LEAEDAAAYYCHQSSSLPHTFGPGTKVEIK |
| | | sequence | |
| 126 | IMPI-055 | LCDR1 | QSIGSS |
| 288 | IMPI-055 | LCDR2 | FAS |
| 128 | IMPI-055 | LCDR3 | HQSSSLPHT |
| 198 | IMPI-049 | VH domain | GAGGTGCAGCTGGTGCAGTCTGGAGCAGAGGTGAAAAAGCCCGGGGAGTCTCTGAAGATCTCCTGTAAGGGTTCTGG |
| | | nucleic | ATACAGCTTTACCAGCTACTGGATCGGCTGGGTGCGCCAGATGCCCGGGAAAGGCCTGGAGTGGATGGGGATCATCT |
| | | acid | ATCCTGGTGACTCTGATACCAGATACAGCCCGTCCTTCCAAGGCCAGGTCACCATCTCAGCCGACAAGTCCATCAGC |
| | | sequence | ACCGCCTACCTGCAGTGGAGCAGCCTGAAGGCCTCGGACACCGCCATGTATTACTGTGTGAGGTCGTATAATTGGAA |
| | | | CTACTTTGACTACTGGGGCCAGGGAACCCTGGTCACCGTCTCCTCA |
| 199 | IMPI-049 | VH domain | EVQLVQSGAEVKKPGESLKISCKGSGYSFTSYWIGWVRQMPGKGLEWMGIIYPGDSDTRYSPSFQGQVTISADKSIS |
| | | amino acid | TAYLQWSSLKASDTAMYYCVRSYNWNYFDYWGQGTLVTVSS |
| | | sequence | |
| 32 | IMPI-049 | HCDR1 | GYSFTSYW |
| 33 | IMPI-049 | HCDR2 | IYPGDSDT |
| 200 | IMPI-049 | HCDR3 | VRSYNWNYFDY |
| 289 | IMPI-049 | VL domain | GCCATCCAGTTGACCCAGTCTCCATCCTCCCTGTCTGCATCTGTAGGAGACAGAGTCACCATCACTTGCCGGGCAAG |
| | | nucleic | TCAGGGCATTAGCAGTGGTTTAGCCTGGTATCAGCAGAAACCAGGGAAAGCTCCTAAGCTCCTGATCTATGATGTCT |
| | | acid | CCAGTTTGGAAAGTGGGGTCCCATCAAGGTTCAGCGGCAGTGGATCTGGGACAGATTTCACTCTCACCATCAGCAGC |
| | | sequence | CTGCAGCCTGAAGATTTTGCAACTTATTACTGTCAACAGTTTCTTAGTTACCCTTGGACGTTCGGCCAAGGGACCAA |
| | | | GGTGGAAATCAAA |
| 267 | IMPI-049 | VL domain | AIQLTQSPSSLSASVGDRVTITCRASQGISSGLAWYQQKPGKAPKLLIYDVSSLESGVPSRFSGSGSGTDFTLTISS |
| | | amino acid | LQPEDFATYYCQQFLSYPWTFGQGTKVEIK |
| | | sequence | |
| 92 | IMPI-049 | LCDR1 | QGISSG |
| 203 | IMPI-049 | LCDR2 | DVS |
| 204 | IMPI-049 | LCDR3 | QQFLSYPWT |
| 290 | IMPI-042 | VH domain | GAGGTGCAACTGGTGGAGTCTGGAGGAGGCTTGATCCAGCCTGGGGGGTCCCTGAGACTCTCCTGTGCAGCCTCTGG |
| | | nucleic | GCTCACCGTCAGTAGCAACTACATGAGCTGGGTCCGCCAGGCTCCAGGGAAGGGGCTGGAGTGGGTCTCAATTATTT |
| | | acid | ATAGCGGTGGTAGCACATACTATGCAGACTCCGTGAAGGGCCGATTCACCATCTCCAGAGACAATTCCAAGAACACG |
| | | sequence | CTGTATCTTCAAATGAACAGCCTGAGAGCCGAGGACACGGCCGTGTATTACTGTGCGAGGCTAGGGGGGTACTACTA |
| | | | CTACGGTATGGACGTCTGGGGCCAAGGGACCACGGTCACCGTCTCCTCA |
| 257 | IMPI-042 | VH domain | EVQLVESGGGLIQPGGSLRLSCAASGLTVSSNYMSWVRQAPGKGLEWVSIIYSGGSTYYADSVKGRFTISRDNSKNT |
| | | amino acid | LYLQMNSLRAEDTAVYYCARLGGYYYYGMDVWGQGTTVTVSS |
| | | sequence | |
| 172 | IMPI-042 | HCDR1 | GLTVSSNY |
| 14 | IMPI-042 | HCDR2 | IYSGGST |
| 258 | IMPI-042 | HCDR3 | ARLGGYYYYGMDV |
| 291 | IMPI-042 | VL domain | GACATCCAGATGACCCAGTCTCCATCCTCCCTGTCTGCATCTGTAGGAGAAAGAGTCACCATCACTTGCCAGGCGAG |
| | | nucleic | TCAGGACATTAGAAACTATTTAAATTGGTATCAGAAGAAACCAGGGAAAGCCCCTAAACTCCTGATCTACGATTCAT |
| | | acid | CCAATTTGGAAACAGGGGTCCCATCAAGGTTCAGTGGAGGTGGATCTGGGACAGATTTTACTTTCACCATCAGCAGC |
| | | sequence | CTGCAGCCTGAAGATATTGCAACATATTACTGTCAACAGTATGATAATCTCCCGATCACCTTCGGCCAAGGGACACG |
| | | | ACTGGAGATTAAA |
| 292 | IMPI-042 | VL domain | DIQMTQSPSSLSASVGERVTITCQASQDIRNYLNWYQKKPGKAPKLLIYDSSNLETGVPSRFSGGGSGTDFTFTISS |
| | | amino acid | LQPEDIATYYCQQYDNLPITFGQGTRLEIK |
| | | sequence | |
| 261 | IMPI-042 | LCDR1 | QDIRNY |
| 262 | IMPI-042 | LCDR2 | DSS |
| 263 | IMPI-042 | LCDR3 | QQYDNLPIT |
| 293 | IMPI-035 | VH domain | GAAGTGCAGCTGGTGGAGTCTGGGGGAGACTTGGTACAGCCTGGCAGGTCCCTGAGACTCTCCTGTGCAGCCTCTGG |
| | | nucleic | ATTCACCTTTGATGATTATGCCATGCACTGGGTCCGGCAAGCTCCAGGGAAGGGCCTGGAGTGGGTCTCAGGTATTA |
| | | acid | GTTGGAATAGTGGTAGCATAGGCTATGCGGACTCTGTGAAGGGCCGATTCACCATCTCCAGAGACAACGCCAAGAAC |
| | | sequence | TCCCTGTATCTGCAAATGAACAGTCTGAGAGCTGAGGACACGGCCTTGTATTACTGTGCAAAAGATCTCCAGGGGGA |
| | | | CTACTACTACTACGGTGTGGACGTCTGGGGCCAAGGGACCACGGTCACCGTCTCCTCA |
| 294 | IMPI-035 | VH domain | EVQLVESGGDLVQPGRSLRLSCAASGFTFDDYAMHWVRQAPGKGLEWVSGISWNSGSIGYADSVKGRFTISRDNAKN |
| | | amino acid | SLYLQMNSLRAEDTALYYCAKDLQGDYYYYGVDVWGQGTTVTVSS |
| | | sequence | |
| 22 | IMPI-035 | HCDR1 | GFTFDDYA |
| 23 | IMPI-035 | HCDR2 | ISWNSGSI |
| 147 | IMPI-035 | HCDR3 | AKDLQGDYYYYGVDV |
| 295 | IMPI-035 | VL domain | GCCATCCAGTTGACCCAGTCTCCATCCTCCCTGTCTGCATCTGTAGGAGACAGAGTCACCATCACTTGCCGGGCAAG |
| | | nucleic | TCAGGGCATTAGCAGTGCTTTAGCCTGGTATCAGCAGAAATCAGGAAAAGCTCCTAAGCTCCTGATCTATGATGCCT |
| | | acid | CCAGTTTGGGAAGTGGGGTCCCATCAAGGTTCAGCGGCAGTAGATCTGGGACAGATTTCACTCTCACCATCAGCAGC |
| | | sequence | CTGCAACCTGAAGATTTTGCAACTTATTACTGTCAACAGTTTATTAGTTACCCGCTCACTTTCGGCGGAGGGACCAA |
| | | | GGTGGAGATCAAA |
| 296 | IMPI-035 | VL domain | AIQLTQSPSSLSASVGDRVTITCRASQGISSALAWYQQKSGKAPKLLIYDASSLGSGVPSRFSGSRSGTDFTLTISS |
| | | amino acid | LQPEDFATYYCQQFISYPLTFGGGTKVEIK |
| | | sequence | |
| 37 | IMPI-035 | LCDR1 | QGISSA |
| 28 | IMPI-035 | LCDR2 | DAS |
| 69 | IMPI-035 | LCDR3 | QQFISYPLT |
| 297 | IMPI-028 | VH domain | GAGGTGCAGCTGGTGGAGTCTGGAGGAGGCTTGATCCAGCCTGGGGGGTCCCTGAGACTCTCCTGTGCAGCCTCTGG |
| | | nucleic | GTTCACCGTCAGTAGCAACTACATGAGCTGGGTCCGCCAGGCTCCAGGGAAGGGGCTGGAGTGGGTCTCAGTTATTT |
| | | acid | ATAGCGGTGGTAGCACATACTACGCAGACTCCGTGAAGGGCCGATTCACCATCTCCAGAGACAATTCCAAGAACACG |
| | | sequence | CTGTATCTTCAAATGAACAGCCTGAGAGCCGAGGACACGGCCGTGTATTACTGTGCGAGAGACTACGGTGACCTATA |
| | | | CTTTGACTACTGGGGCCAGGGAACCCTGGTCACCGTCTCCTCA |
| 298 | IMPI-028 | VH domain | EVQLVESGGGLIQPGGSLRLSCAASGFTVSSNYMSWVRQAPGKGLEWVSVIYSGGSTYYADSVKGRFTISRDNSKNT |
| | | amino acid | LYLQMNSLRAEDTAVYYCARDYGDLYFDYWGQGTLVTVSS |
| | | sequence | |
| 13 | IMPI-028 | HCDR1 | GFTVSSNY |
| 14 | IMPI-028 | HCDR2 | IYSGGST |
| 299 | IMPI-028 | HCDR3 | ARDYGDLYFDY |
| 300 | IMPI-028 | VL domain | GAAATTGTGTTGACGCAGTCTCCAGGCACCCTGTCTTTGTCTCCAGGGGAAAGAGCCACCCTCTCCTGCAGGGCCAG |
| | | nucleic | TCAGAGTATTAGCAGCTACTTAGCCTGGTACCAGCAGAAACCTGGCCAGGCTCCCAGGCTCCTCATCTATGGTGCAT |
| | | acid | CCAGCAGGGCCACTGGCATCCCAGACAAATTCAGTGGCAGTGGGTCTGGGACAGACTTCACTCTCACCATCAGCAGA |
| | | sequence | CTGGAGCCTGAAGATTTTGCAGTGTATTACTGTCAGCAGTATGGTAGCTCACCTCGGACGTTCGGCCAAGGGACCAA |
| | | | GGTGGAAATCAAA |
| 301 | IMPI-028 | VL domain | EIVLTQSPGTLSLSPGERATLSCRASQSISSYLAWYQQKPGQAPRLLIYGASSRATGIPDKFSGSGSGTDFTLTISR |
| | | amino acid | LEPEDFAVYYCQQYGSSPRTFGQGTKVEIK |
| | | sequence | |
| 302 | IMPI-028 | LCDR1 | QSISSY |
| 157 | IMPI-028 | LCDR2 | GAS |
| 303 | IMPI-028 | LCDR3 | QQYGSSPRT |
| 304 | IMPI-018 | VH domain | CAGGTGCAGCTGCAGGAGTCGGGCCCAGGACTGGTGAAGCCTTCACAGACCCTGTCCCTCACCTGCACTGTCTCTGG |
| | | nucleic | TGACTCCATCAGCCGTGGTGGTTTCTACTGGAGCTGGATCCGCCAGCACCCAGGGAAGGGCCTGGAGTGGATTGGGT |
| | | acid | ACATCTATTACAGTGGGAGCACCTACTACAACCCGTCCCTCAAGGGTCGAGTTACCATATCAGTAGACACGTCTAAG |
| | | sequence | AACCAGTTCTCCCTGAAGCTGACCTCTGTGACTGCCGCGGACACGGCCGTGTATTACTGTGCGAGAGACTACGGTGG |
| | | | TAACTCGAACTACTTTGACTACTGGGGCCAGGGGACCCTGGTCACCGTCTCCTCA |
| 305 | IMPI-018 | VH domain | QVQLQESGPGLVKPSQTLSLTCTVSGDSISRGGFYWSWIRQHPGKGLEWIGYIYYSGSTYYNPSLKGRVTISVDTSK |
| | | amino acid | NQFSLKLTSVTAADTAVYYCARDYGGNSNYFDYWGQGTLVTVSS |
| | | sequence | |
| 306 | IMPI-018 | HCDR1 | GDSISRGGFY |
| 42 | IMPI-018 | HCDR2 | IYYSGST |
| 307 | IMPI-018 | HCDR3 | ARDYGGNSNYFDY |
| 308 | IMPI-018 | VL domain | GCCATCCAGTTGACCCAGTCTCCATCCTCCCTGTCTGCATCTGTAGGAGACAGAGTCACCATCACTTGCCGGGCAAG |
| | | nucleic | TCAGGGCATTAGCAGTGCTTTAGCCTGGTATCAGCAGAAACCAGGGAAAGCTCCTAAGCTCCTGATCTATGATGCCT |
| | | acid | CCAGTTTGGAAAGTGGGGTCCCATCAAGGTTCAGCGGCAGTGGATCTGGGACAGATTTCACTCTCACCATCAGCAGC |
| | | sequence | CTGCAGCCTGAAGATTTTGCACCTTATTACTGTCAACAGTTTAATAGTTACCCTCTCACTTTCGGCGGAGGGACCAA |
| | | | GGTGGAGATCAGG |
| 309 | IMPI-018 | VL domain | AIQLTQSPSSLSASVGDRVTITCRASQGISSALAWYQQKPGKAPKLLIYDASSLESGVPSRFSGSGSGTDFTLTISS |
| | | amino acid | LQPEDFAPYYCQQFNSYPLTFGGGTKVEIR |
| | | sequence | |
| 37 | IMPI-018 | LCDR1 | QGISSA |
| 28 | IMPI-018 | LCDR2 | DAS |
| 47 | IMPI-018 | LCDR3 | QQFNSYPLT |
| 264 | IMPI-050 | VH domain | GAGGTGCAGCTGGTGCAGTCTGGAGCAGAGGTGAAAAAGCCCGGGGAGTCTCTGAAGATCTCCTGTAAGGGTTCTGG |
| | | nucleic | ATACAGCTTTACCAGCTACTGGATCGGCTGGGTGCGCCAGATGCCCGGGAAAGGCCTGGAGTGGATGGGGATCATCT |
| | | acid | ATCCTGGTGACTCTGATACCAGATACAGCCCGTCCTTCCAAGGCCAGGTCACCATCTCAGCCGACAAGTCCATCAGC |
| | | sequence | ACCGCCTACCTGCAGTGGAGCAGCCTGAAGGCCTCGGACACCGCCATGTATTACTGTGCGAGGTCGTATAACTGGAA |
| | | | CTACTTTGACTACTGGGGCCAGGGAACCCTGGTCACCGTCTCCTCA |
| 265 | IMPI-050 | VH domain | EVQLVQSGAEVKKPGESLKISCKGSGYSFTSYWIGWVRQMPGKGLEWMGIIYPGDSDTRYSPSFQGQVTISADKSIS |
| | | amino acid | TAYLQWSSLKASDTAMYYCARSYNWNYFDYWGQGTLVTVSS |
| | | sequence | |
| 32 | IMPI-050 | HCDR1 | GYSFTSYW |
| 33 | IMPI-050 | HCDR2 | IYPGDSDT |
| 34 | IMPI-050 | HCDR3 | ARSYNWNYFDY |
| 310 | IMPI-050 | VL domain | GCCATCCAGTTGACCCAGTCTCCATCCTCCCTGTCTGCATCTGTAGGAGACAGAGTCACCATCACTTGCCGGGCAAG |
| | | nucleic | TCAGGGCATTAACAATGGTTTAGCCTGGTATCAGCAGAAACCAGGGAAAGCTCCTAAGCTCCTGATCTATGATGTCT |
| | | acid | CCAGTTTGGAAAGTGGGGTCCCATCAAGGTTCAGCGGCAGTGGATCTGGGAGAGATTCCACTCTCACCATCAGCAGT |
| | | sequence | CTGCAGTCTGAAGATTTTGCAACTTATTACTGTCAACAGTTTAAAAGTTACCCTTGGACGTTCGGCCAAGGGACCAA |
| | | | GGTGGAAATCAAA |
| 311 | IMPI-050 | VL domain | AIQLTQSPSSLSASVGDRVTITCRASQGINNGLAWYQQKPGKAPKLLIYDVSSLESGVPSRFSGSGSGRDSTLTISS |
| | | amino acid | LQSEDFATYYCQQFKSYPWTFGQGTKVEIK |
| | | sequence | |
| 312 | IMPI-050 | LCDR1 | QGINNG |
| 203 | IMPI-050 | LCDR2 | DVS |
| 93 | IMPI-050 | LCDR3 | QQFKSYPWT |
| 264 | IMPI-016 | VH domain | GAGGTGCAGCTGGTGCAGTCTGGAGCAGAGGTGAAAAAGCCCGGGGAGTCTCTGAAGATCTCCTGTAAGGGTTCTGG |
| | | nucleic | ATACAGCTTTACCAGCTACTGGATCGGCTGGGTGCGCCAGATGCCCGGGAAAGGCCTGGAGTGGATGGGGATCATCT |
| | | acid | ATCCTGGTGACTCTGATACCAGATACAGCCCGTCCTTCCAAGGCCAGGTCACCATCTCAGCCGACAAGTCCATCAGC |
| | | sequence | ACCGCCTACCTGCAGTGGAGCAGCCTGAAGGCCTCGGACACCGCCATGTATTACTGTGCGAGGTCGTATAACTGGAA |
| | | | CTACTTTGACTACTGGGGCCAGGGAACCCTGGTCACCGTCTCCTCA |
| 265 | IMPI-016 | VH domain | EVQLVQSGAEVKKPGESLKISCKGSGYSFTSYWIGWVRQMPGKGLEWMGIIYPGDSDTRYSPSFQGQVTISADKSIS |
| | | amino acid | TAYLQWSSLKASDTAMYYCARSYNWNYFDYWGQGTLVTVSS |
| | | sequence | |
| 32 | IMPI-016 | HCDR1 | GYSFTSYW |
| 33 | IMPI-016 | HCDR2 | IYPGDSDT |
| 34 | IMPI-016 | HCDR3 | ARSYNWNYFDY |
| 313 | IMPI-016 | VL domain | GCCATCCAGTTGACCCAGTCTCCATCCTCCCTGTCTGCATCTGTAGGAGACAGAGTCACCATCACTTGCCGGGCAAG |
| | | nucleic | TCAGGGCATTAGCAGTGGTTTAGCCTGGTATCAGCAGAAACCAGGGAAAGCTCCTAAGCTCCTGATCTATGATGCCT |
| | | acid | CCAGTTTGGAAAGTGGGGTCCCATCAAGGTTCAGCGGCAGTGGATCTGGGACAGATTTCACTCTCACCATCAGCAAC |
| | | sequence | CTGCAGCCTGAAGATTTTGCAACTTATTACTGTCAACAGTTTAATAGTTACCCTTGGACGTTCGGCCAAGGGACCAA |
| | | | GGTGGAAATCAAA |
| 314 | IMPI-016 | VL domain | AIQLTQSPSSLSASVGDRVTITCRASQGISSGLAWYQQKPGKAPKLLIYDASSLESGVPSRFSGSGSGTDFTLTISN |
| | | amino acid | LQPEDFATYYCQQFNSYPWTFGQGTKVEIK |
| | | sequence | |
| 92 | IMPI-016 | LCDR1 | QGISSG |
| 28 | IMPI-016 | LCDR2 | DAS |
| 76 | IMPI-016 | LCDR3 | QQFNSYPWT |
| 315 | IMPI-040 | VH domain | CAGGTGCAGCTACAGGAGTCGGGCCCAGGACTGGTGAAGCCTTCGGGGACCCTGTCCCTCACCTGCGCTGTCTCTGG |
| | | nucleic | TGGCTCCATCAGCAGTAATAACTGGTGGAGTTGGGTCCGCCAGCCCCCAGGGAAGGGTCTGGAGTGGATTGGGGAAA |
| | | acid | TCTATCATAGTGGGAGCACCATGTACAACCCGTCCCTCAAGAGTCGAGTCACCATATCAGTAGACAAGTCCAAGAAC |
| | | sequence | CAGTTCTCCCTGAAGCTGAACTCTGTGACCGCCGCGGACACGGCCGTGTATTACTGTACGGGAGAGGGGTCTAACTG |
| | | | GAACTACTGGGGCCAGGGGACCCTGGTCACCGTCTCCTCA |
| 316 | IMPI-040 | VH domain | QVQLQESGPGLVKPSGTLSLTCAVSGGSISSNNWWSWVRQPPGKGLEWIGEIYHSGSTMYNPSLKSRVTISVDKSKN |
| | | amino acid | QFSLKLNSVTAADTAVYYCTGEGSNWNYWGQGTLVTVSS |
| | | sequence | |
| 131 | IMPI-040 | HCDR1 | GGSISSNNW |
| 140 | IMPI-040 | HCDR2 | IYHSGST |
| 317 | IMPI-040 | HCDR3 | TGEGSNWNY |
| 318 | IMPI-040 | VL domain | GATGTTGTGATGACTCAGTCTCCACTCTCCCTGCCCGTCACCCTTGGACAGCCGGCCTCCATCTCCTGCAGGTCTAG |
| | | nucleic | TCAAAGCCTCGTATATACTGATGGAAACACCTACTTGAGTTGGTTTCAGCAGAGGCCAGGCCAATCTCCAAGGCGCC |
| | | acid | TACTTTATAAGGTTTCTAACTGGGACTCTGGGGTCCCAGACAGATTCAGCGGCAGTGGGTCAGGCACTGATTTCACA |
| | | sequence | CTGAAAATCAGCAGGGTGGAGGCTGAAGATGTTGGGATTTATTATTGCATGCAAGGTACACACTGGCCGCTCACTTT |
| | | | CGGCGGAGGGACCAAGGTGGAGATCAAA |
| 319 | IMPI-040 | VL domain | DVVMTQSPLSLPVTLGQPASISCRSSQSLVYTDGNTYLSWFQQRPGQSPRRLLYKVSNWDSGVPDRFSGSGSGTDFT |
| | | amino acid | LKISRVEAEDVGIYYCMQGTHWPLTFGGGTKVEIK |
| | | sequence | |
| 144 | IMPI-040 | LCDR1 | QSLVYTDGNTY |
| 85 | IMPI-040 | LCDR2 | KVS |
| 86 | IMPI-040 | LCDR3 | MQGTHWPLT |
| 77 | IMPI-030 | VH domain | CAGGTGCAGCTGCAGGAGTCGGGCCCAGGACTGGTGAAGCCTTCGGGGACCCTGTCCCTCACCTGCGCTGTCTCTGG |
| | | nucleic | TGGCTCCATCAGCAGGAGTACCTGGTGGAGTTGGGTCCGCCAGCCCCCAGGGAAGGGGCTGGAGTGGATTGGGGAAA |
| | | acid | TCTCTCATAGTGGGAGCACCCAGTACAACCCGTCCCTCAAGAGTCGAGTCACCATATCACTAGACAAGTCCAAGAAC |
| | | sequence | CAGTTCTCCCTGAAGCTGAACTCTGTGACCGCCGCGGACACGGCCGTATATTACTGTGCGGGAGAGGGGTATAACTG |
| | | | GAACTACTGGGGCCAGGGAACCCTGGTCACCGTCTCCTCA |
| 78 | IMPI-030 | VH domain | QVQLQESGPGLVKPSGTLSLTCAVSGGSISRSTWWSWVRQPPGKGLEWIGEISHSGSTQYNPSLKSRVTISLDKSKN |
| | | amino acid | QFSLKLNSVTAADTAVYYCAGEGYNWNYWGQGTLVTVSS |
| | | sequence | |
| 79 | IMPI-030 | HCDR1 | GGSISRSTW |
| 80 | IMPI-030 | HCDR2 | ISHSGST |
| 81 | IMPI-030 | HCDR3 | AGEGYNWNY |
| 320 | IMPI-030 | VL domain | GATGTTGTAATGACTCAGTCTCCACTCTCCCTGCCCGTCACCCTTGGACAGCCGGCCTCCATCTCCTGCAGGTCTAG |
| | | nucleic | TCAAAGCCTCGTATACAGTGATGGAAACACCTACTTGAGTTGGTTTCAGCAGAGGCCAGGCCAATCTCCAAGGCGCC |
| | | acid | TAATTTATAAGGTTTCTAAGTGGGACTCTGGGGTCCCAGACAGATTCAGCGGCAGTGGGTCAGGCACTGATTTCACA |
| | | sequence | CTGAAAATCAGCAGGGTGGAGGCTGAGGATGTTGGGGTTTATTACTGCATGCAAGGTACACACTGGCCGCTCACTTT |
| | | | CGGCGGAGGGACCAAGGTGGAGATCAAA |
| 321 | IMPI-030 | VL domain | DVVMTQSPLSLPVTLGQPASISCRSSQSLVYSDGNTYLSWFQQRPGQSPRRLIYKVSKWDSGVPDRFSGSGSGTDFT |
| | | amino acid | LKISRVEAEDVGVYYCMQGTHWPLTFGGGTKVEIK |
| | | sequence | |
| 84 | IMPI-030 | LCDR1 | QSLVYSDGNTY |
| 85 | IMPI-030 | LCDR2 | KVS |
| 86 | IMPI-030 | LCDR3 | MQGTHWPLT |
| 264 | IMPI-034 | VH domain | GAGGTGCAGCTGGTGCAGTCTGGAGCAGAGGTGAAAAAGCCCGGGGAGTCTCTGAAGATCTCCTGTAAGGGTTCTGG |
| | | nucleic | ATACAGCTTTACCAGCTACTGGATCGGCTGGGTGCGCCAGATGCCCGGGAAAGGCCTGGAGTGGATGGGGATCATCT |
| | | acid | ATCCTGGTGACTCTGATACCAGATACAGCCCGTCCTTCCAAGGCCAGGTCACCATCTCAGCCGACAAGTCCATCAGC |
| | | sequence | ACCGCCTACCTGCAGTGGAGCAGCCTGAAGGCCTCGGACACCGCCATGTATTACTGTGCGAGGTCGTATAACTGGAA |
| | | | CTACTTTGACTACTGGGGCCAGGGAACCCTGGTCACCGTCTCCTCA |
| 265 | IMPI-034 | VH domain | EVQLVQSGAEVKKPGESLKISCKGSGYSFTSYWIGWVRQMPGKGLEWMGIIYPGDSDTRYSPSFQGQVTISADKSIS |
| | | amino acid | TAYLQWSSLKASDTAMYYCARSYNWNYFDYWGQGTLVTVSS |
| | | sequence | |
| 32 | IMPI-034 | HCDR1 | GYSFTSYW |
| 33 | IMPI-034 | HCDR2 | IYPGDSDT |
| 34 | IMPI-034 | HCDR3 | ARSYNWNYFDY |
| 322 | IMPI-034 | VL domain | GCCATCCAGTTGACCCAGTCTCCATCCTCCCTGTCTGCATCTGTAGGAGACAGAGTCACCATCACTTGCCGGGCAAG |
| | | nucleic | TCAGGGCATTAACAGTGGTTTAGCCTGGTATCAGCAGAAACCAGGGAAAGGTCTTAAGCTCCTGATCTATGATGTCT |
| | | acid | CCAGTTTGGAAAGTGGGGTCCCATCAAGGTTCAGCGGCAGTGGATCTGGGAGAGATTTCACTCTCACCATCAGCAGC |
| | | sequence | CTGCAGCCTGAAGATTTTGCAACTTATTACTGTCAACAGTTTAAAAGTTACCCTTGGACGTTCGGCCAAGGGACCAA |
| | | | GGTGGAAATCAAA |
| 323 | IMPI-034 | VL domain | AIQLTQSPSSLSASVGDRVTITCRASQGINSGLAWYQQKPGKGLKLLIYDVSSLESGVPSRFSGSGSGRDFTLTISS |
| | | amino acid | LQPEDFATYYCQQFKSYPWTFGQGTKVEIK |
| | | sequence | |
| 324 | IMPI-034 | LCDR1 | QGINSG |
| 203 | IMPI-034 | LCDR2 | DVS |
| 93 | IMPI-034 | LCDR3 | QQFKSYPWT |
| 325 | IMPI-013 | VH domain | GAAGTGCAGCTGGTGGAGTCTGGGGGAGGCTTGGTACAGCCTGGCAGGTCCCTGAGACTCTCCTGTGCAGCCTCTGG |
| | | nucleic | ATTCACCTTTGATGATTATGCCATGCACTGGGTCCGGCAAACTCCAGGGAAGGGCCTGGAGTGGGTCTCAGGTATTA |
| | | acid | GTTGGAATAGTGGTAGCATAGGCTATGCGGACTCTGTGAAGGGCCGATTCACCATCTCCAGAGACAACGCCAAGAAC |
| | | sequence | TCCCTGTATCTGCAAATGAACAGTCTGAGAGCTGAAGACACGGCCTTATATTACTGTACAAAAGATTTGGGACTGGG |
| | | | GATTGGCTTCTACTACGGTTTGGACGTCTGGGGCCAGGGGACCACGGTCACCGTCTCCTCA |
| 326 | IMPI-013 | VH domain | EVQLVESGGGLVQPGRSLRLSCAASGFTFDDYAMHWVRQTPGKGLEWVSGISWNSGSIGYADSVKGRFTISRDNAKN |
| | | amino acid | SLYLQMNSLRAEDTALYYCTKDLGLGIGFYYGLDVWGQGTTVTVSS |
| | | sequence | |
| 22 | IMPI-013 | HCDR1 | GFTFDDYA |
| 23 | IMPI-013 | HCDR2 | ISWNSGSI |
| 327 | IMPI-013 | HCDR3 | TKDLGLGIGFYYGLDV |
| 328 | IMPI-013 | VL domain | GCCATCCAAATGACCCAGTCTCCATCCTCCCTGTCTACATCTGTAGGAGACAGAGTCACCATCACTTGCCGGGCAAG |
| | | nucleic | TCAGGGCATTAGAAATGATTTAGGCTGGTATCAGCTGAAGCCAGGGAAAGCCCCTAAGCTCCTGATCTATGATGCAT |
| | | acid | CCACTTTACAAAGTGGGGTCCCATCGAGGTTCAGCGGCAGTGGATCTGGCACAGATTTCACTCTCACCATCAGCAGC |
| | | sequence | CTGGAGCCTGAAGATTTAGCAACTTATTACTGTCTACATCACTACACTTACCCGTGGACGTTCGGCCATGGGACCAA |
| | | | GGTGGAACTCAAA |
| 329 | IMPI-013 | VL domain | AIQMTQSPSSLSTSVGDRVTITCRASQGIRNDLGWYQLKPGKAPKLLIYDASTLQSGVPSRFSGSGSGTDFTLTISS |
| | | amino acid | LEPEDLATYYCLHHYTYPWTFGHGTKVELK |
| | | sequence | |
| 27 | IMPI-013 | LCDR1 | QGIRND |
| 28 | IMPI-013 | LCDR2 | DAS |
| 29 | IMPI-013 | LCDR3 | LHHYTYPWT |
| 330 | IMPI-026 | VH domain | GAGGTGCAGCTGGTGCAGTCTGGAGCAGAGGTGAAAAAGCCCGGGGAGTCTCTGAAGATCTCCTGTAAGGGTTTTGG |
| | | nucleic | ATACAGCTTTACCAGCTACTGGATCGGCTGGGTGCGCCAGATGCCCGGGAAAGGCCTGGAGTGGATGGGGATCATCT |
| | | acid | ATCCTGGTGACTCTGATACCAGATACAGCCCGTCCTTCCAAGGCCAGGTCACCATCTCAGCCGACAAGTCCATCAGC |
| | | sequence | ACCGCCTACCTGCAGTGGAGCAGCCTGAAGGCCTCGGACACCGCCATGTATTACTGTGCGAGGTCGTATAACTGGAA |
| | | | CTACTTTGACTACTGGGGCCAGGGAACCCTGGTCACCGTCTCCTCA |
| 331 | IMPI-026 | VH domain | EVQLVQSGAEVKKPGESLKISCKGFGYSFTSYWIGWVRQMPGKGLEWMGIIYPGDSDTRYSPSFQGQVTISADKSIS |
| | | amino acid | TAYLQWSSLKASDTAMYYCARSYNWNYFDYWGQGTLVTVSS |
| | | sequence | |
| 32 | IMPI-026 | HCDR1 | GYSFTSYW |
| 33 | IMPI-026 | HCDR2 | IYPGDSDT |
| 34 | IMPI-026 | HCDR3 | ARSYNWNYFDY |
| 332 | IMPI-026 | VL domain | GCCATCCAGTTGACCCAGTCTCCATCCTCCCTGTCTGCATCTGTAGGAGACAGAGTCACCATCACTTGCCGGGCAAG |
| | | nucleic | TCAGGGCATTAGCAGTGCTTTAGCCTGGTATCAGCAGAAACCAGGGAAAGCTCCTAAGCTCCTGATCTATGATGCCT |
| | | acid | CCAGTTTGGAAAGTGGGGTCCCATCAAGGTTCAGCGGCAGTGGATCTGGGACAGATTTCACTCTCACCATCAGCAGC |
| | | sequence | CTGCAGCCTGAGGATTTTGCAACTTATTACTGTCAACAGTTTAATAGTTACCCTTGGACGTTCGGCCAAGGGACCAA |
| | | | GGTGGAAATCAAA |
| 333 | IMPI-026 | VL domain | AIQLTQSPSSLSASVGDRVTITCRASQGISSALAWYQQKPGKAPKLLIYDASSLESGVPSRFSGSGSGTDFTLTISS |
| | | amino acid | LQPEDFATYYCQQFNSYPWTFGQGTKVEIK |
| | | sequence | |
| 37 | IMPI-026 | LCDR1 | QGISSA |
| 28 | IMPI-026 | LCDR2 | DAS |
| 76 | IMPI-026 | LCDR3 | QQFNSYPWT |
| 334 | IMPI-007 | VH domain | CAGGTGCAGCTGCAGGAGTCGGGCCCAGGACTGGTGAAGCCTTCGGGGACCCTGTCCCTCACCTGCGCTGTCTCGAG |
| | | nucleic | TGGCTCCATCAGCAGAAGTACCTGGTGGAGTTGGGTCCGCCAGCCCCCAGGGAAGGGGCTGGAGTGGATTGGGGAAA |
| | | acid | TCTATCATAGTGGGAGCACCCAATACAACCCGTCCCTCAAGAGTCGAGTCACCATATCAGTAGACAAGTCCAAGAAC |
| | | sequence | CAGTTCTCCCTGAAGCTGAGTTCTGCGACCGCCGCGGACACGGCCGTGTATTACTGTGCGGGAGAGGGGTCTAACTG |
| | | | GAACTACTGGGGCCAGGGAACCCTGGTCACCGTCTCCTCA |
| 335 | IMPI-007 | VH domain | QVQLQESGPGLVKPSGTLSLTCAVSSGSISRSTWWSWVRQPPGKGLEWIGEIYHSGSTQYNPSLKSRVTISVDKSKN |
| | | amino acid | QFSLKLSSATAADTAVYYCAGEGSNWNYWGQGTLVTVSS |
| | | sequence | |
| 336 | IMPI-007 | HCDR1 | SGSISRSTW |
| 140 | IMPI-007 | HCDR2 | IYHSGST |
| 337 | IMPI-007 | HCDR3 | AGEGSNWNY |
| 338 | IMPI-007 | VL domain | GATGTTGTGATGACTCAGTCTCCACTCTCCCTGCCCGTCACCCTTGGACAGCCGGCCTCCATCTCCTGCAGGTCTAG |
| | | nucleic | TCAAAGCCTCGTATACAGTGATGGAAACACCTACTTGAGTTGGTTTCAGCAGAGGCCAGGCCAATCTCCAAGGCGCC |
| | | acid | TAATTTATAAGGTTTCTAACTGGGACTCTGGGGTCCCAGACAGATTCAGCGGCAGTGGGTCAGGCACTGATTTCACA |
| | | sequence | CTGAGAATCAGCAGGGTGGAGGCTGAGGATATTGGGGTTTATTACTGCATGCAAGGTACACACTGGCCGCTCACTTT |
| | | | CGGCGGAGGGACCAAGGTGGAGCTCAAA |
| 339 | IMPI-007 | VL domain | DVVMTQSPLSLPVTLGQPASISCRSSQSLVYSDGNTYLSWFQQRPGQSPRRLIYKVSNWDSGVPDRFSGSGSGTDFT |
| | | amino acid | LRISRVEAEDIGVYYCMQGTHWPLTFGGGTKVELK |
| | | sequence | |
| 84 | IMPI-007 | LCDR1 | QSLVYSDGNTY |
| 85 | IMPI-007 | LCDR2 | KVS |
| 86 | IMPI-007 | LCDR3 | MQGTHWPLT |
| 340 | IMPI-046 | VH domain | GAGGTGCAGCTGGTGCAGTCTGGAGCAGAGGTGAAAAAGCCCGGGGAGTCTCTGAAGATCTCCTGTAAGGGTTCTGG |
| | | nucleic | ATACAGATTTACCAGTTACTGGATCGGCTGGGTGCGCCAGATGCCCGGGAAAGGCCTGGAGTGGATGGGGATCATCT |
| | | acid | ATCCTGGTGACTCTGATACCAGATACAGCCCGTCCTTCCAAGGCCAGGTCACCATCTCAGCCGACAAGTCCATCAGC |
| | | sequence | ACCGCCTACCTGCAGTGGAGCAGCCTGAAGGCCTCGGACACCGCCATGTATTACTGTGCGAGGTCGTATAACTGGAA |
| | | | CTACTTTGACTACTGGGGCCAGGGAACCCTGGTCACCGTCTCCTCA |
| 341 | IMPI-046 | VH domain | EVQLVQSGAEVKKPGESLKISCKGSGYRFTSYWIGWVRQMPGKGLEWMGIIYPGDSDTRYSPSFQGQVTISADKSIS |
| | | amino acid | TAYLQWSSLKASDTAMYYCARSYNWNYFDYWGQGTLVTVSS |
| | | sequence | |
| 342 | IMPI-046 | HCDR1 | GYRFTSYW |
| 33 | IMPI-046 | HCDR2 | IYPGDSDT |
| 34 | IMPI-046 | HCDR3 | ARSYNWNYFDY |
| 343 | IMPI-046 | VL domain | GCCATCCAGTTGACCCAGTCTCCATCCTCCCTGTCTGCATCTGTAGGAGACAGAGTCACCATCACTTGCCGGGCAAG |
| | | nucleic | TCAGGGCATTAGCAGTGCTTTAGCCTGGTATCAGCAGAAACCAGGGAAAGCTCCTAAGCTCCTGATCTATGATGCCT |
| | | acid | CCAGTTTGGAAAGTGGGGTCCCACCAAGGTTCAGCGGCAGTGGATCTGGGACAGATTTCACTCTCACCATCAGCAGC |
| | | sequence | CTGCAGCCTGAAGATTTTGCAACTTATTACTGTCAACAGTTTATTAGTTACCCTTGGACGTTCGGCCAAGGGACCAA |
| | | | GGTGGAAATCAAA |
| 344 | IMPI-046 | VL domain | AIQLTQSPSSLSASVGDRVTITCRASQGISSALAWYQQKPGKAPKLLIYDASSLESGVPPRFSGSGSGTDFTLTISS |
| | | amino acid | LQPEDFATYYCQQFISYPWTFGQGTKVEIK |
| | | sequence | |
| 37 | IMPI-046 | LCDR1 | QGISSA |
| 28 | IMPI-046 | LCDR2 | DAS |
| 38 | IMPI-046 | LCDR3 | QQFISYPWT |
| 220 | IMPI-060 | VH domain | GAGGTGCAGCTGGTGGAGTCTGGAGGAGGCTTGATCCAGCCTGGGGGGTCCCTGAGACTCTCCTGTGCAGCCTCTGG |
| | | nucleic | GGTCACCGTCAGTAGCAACTACATGAGCTGGGTCCGCCAGGCTCCAGGGAAGGGGCTGGAGTGGGTCTCAGTTATTT |
| | | acid | ATAGCGGTGGTAGCACATTCTACGCAGACTCCGTGAAGGGCCGATTCACCATCTCCAGAGACAATTCCAAGAACACG |
| | | sequence | CTGTATCTTCAAATGAACAGCCTGAGAGCCGAGGACACGGCCGTGTATTACTGTGCGAGAGATCTCTCCTACTACGG |
| | | | TATGGACGTCTGGGGCCAAGGGACCACGGTCACCGTCTCCTCA |
| 221 | IMPI-060 | VH domain | EVQLVESGGGLIQPGGSLRLSCAASGVTVSSNYMSWVRQAPGKGLEWVSVIYSGGSTFYADSVKGRFTISRDNSKNT |
| | | amino acid | LYLQMNSLRAEDTAVYYCARDLSYYGMDVWGQGTTVTVSS |
| | | sequence | |
| 3 | IMPI-060 | HCDR1 | GVTVSSNY |
| 14 | IMPI-060 | HCDR2 | IYSGGST |
| 222 | IMPI-060 | HCDR3 | ARDLSYYGMDV |
| 345 | IMPI-060 | VL domain | GACATCCAGTTGACCCAGTCTCCATCCTTCCTGTCTGCATCTGTAGGAGACAGAGTCACCATCACTTGCTGGGCCAG |
| | | nucleic | TCAGGGCATTAGCAGTTATTTAGCCTGGTATCAGCAAAAACCAGGGAAAGCCCCTAAGCTCCTGATCTATGCTGCAT |
| | | acid | CCACTTTGCAAAGTGGGGTCCCATCAAAATTCAGCGGCAGTGGATCTGGGACAGAATTCACTCTCACAATCAGCAGC |
| | | sequence | CTGCAGCCTGAAGATTTTGCAACTTATTACTGTCAACAGCTTAATAGTTACCCGAGCAGTTTTGGCCAGGGGACCAA |
| | | | GCTGGAGATCAAA |
| 346 | IMPI-060 | VL domain | DIQLTQSPSFLSASVGDRVTITCWASQGISSYLAWYQQKPGKAPKLLIYAASTLQSGVPSKFSGSGSGTEFTLTISS |
| | | amino acid | LQPEDFATYYCQQLNSYPSSFGQGTKLEIK |
| | | sequence | |
| 8 | IMPI-060 | LCDR1 | QGISSY |
| 18 | IMPI-060 | LCDR2 | AAS |
| 347 | IMPI-060 | LCDR3 | QQLNSYPSS |
| 348 | IMPI-056 | VH domain | GAGGTGCAGCTGGTGGAGTCTGGAGGAGGCTTGATCCAGCCTGGGGGGTCCCTGAGACTCTCCTGTGCAGCCTCTGG |
| | | nucleic | GCTCACCGTCAGTAGCAACTACATGAACTGGGTCCGCCAGGCTCCAGGGAAAAAACTGGAGTGGGTCTCAGTTATTT |
| | | acid | ATAGCGGAGGTAGCACATTTTACGCAGACTCCGTGAGGGGCCGATTCACCATCTCCAGAGACAATTCCAAGAACACG |
| | | sequence | CTGTTTCTTCAAATGAACAGCCTGAGAGCCGAGGACTCGGCCGTGTATTACTGTGCGAGAGATTTGGGACCCTACGG |
| | | | TGTGGACGTCTGGGGCCAAGGGACCACGGTCACCGTCTCCTCA |
| 349 | IMPI-056 | VH domain | EVQLVESGGGLIQPGGSLRLSCAASGLTVSSNYMNWVRQAPGKKLEWVSVIYSGGSTFYADSVRGRFTISRDNSKNT |
| | | amino acid | LFLQMNSLRAEDSAVYYCARDLGPYGVDVWGQGTTVTVSS |
| | | sequence | |
| 172 | IMPI-056 | HCDR1 | GLTVSSNY |
| 14 | IMPI-056 | HCDR2 | IYSGGST |
| 106 | IMPI-056 | HCDR3 | ARDLGPYGVDV |
| 350 | IMPI-056 | VL domain | GACATCCAGTTGACCCAGTCTCCATCCTTCCTGTCTGCATCTGTAGGAGACAGAGTCACCATCACTTGCTGGGCCAG |
| | | nucleic | TCAGGGCATTAGCAGTTATTTAGCCTGGTATCAGCAAAAACCAGGGAAAGCCCCTAAACTCCTGATCTATGCTGCAT |
| | | acid | CCACTTTGCAAAGTGGGGTCCCATCAAGGTTCAGCGGCAGTGGATCTGGGACAGAATTCACTCTCACAATCAGCAGC |
| | | sequence | CTGCAGCCTGAAGATTTTGCAACTTATTACTGTCAACAGCTTAATAGTTACCCTCTCACCTTCGGCCAAGGGACACG |
| | | | ACTGGAGATTAAA |
| 351 | IMPI-056 | VL domain | DIQLTQSPSFLSASVGDRVTITCWASQGISSYLAWYQQKPGKAPKLLIYAASTLQSGVPSRFSGSGSGTEFTLTISS |
| | | amino acid | LQPEDFATYYCQQLNSYPLTFGQGTRLEIK |
| | | sequence | |
| 8 | IMPI-056 | LCDR1 | QGISSY |
| 18 | IMPI-056 | LCDR2 | AAS |
| 10 | IMPI-056 | LCDR3 | QQLNSYPLT |
| 352 | IMPI-061 | VH domain | GAGGTGCAGCTGGTGGAGTCTGGGGGAGGTGTGGTACGGCCTGGGGGGTCCCTGAGACTCTCCTGTGCAGCCTCTGG |
| | | nucleic | ATTCACCTTTGATGATTATGGCATGAGCTGGGTCCGCCAAGCTCCAGGGAAGGGGCTGGAGTGGGTCTCTGGTATTT |
| | | acid | ATTGGAATGGTGGTAGCACAGGTTATGCAGACTCTGTGAAGGGCCGATTCACCATCTCCAGAGACAACGCCAAGAAC |
| | | sequence | TCCCTGTATCTGCAAATGAACAGTCTGAGAGCCGAGGACACGGCCTTGTATTACTGTGCGAGAGGAGTGGGAGCTAC |
| | | | AGACTACTGGGGCCAGGGAACCCTGGTCACCGTCTCCTCA |
| 353 | IMPI-061 | VH domain | EVQLVESGGGVVRPGGSLRLSCAASGFTFDDYGMSWVRQAPGKGLEWVSGIYWNGGSTGYADSVKGRFTISRDNAKN |
| | | amino acid | SLYLQMNSLRAEDTALYYCARGVGATDYWGQGTLVTVSS |
| | | sequence | |
| 354 | IMPI-061 | HCDR1 | GFTFDDYG |
| 355 | IMPI-061 | HCDR2 | IYWNGGST |
| 356 | IMPI-061 | HCDR3 | ARGVGATDY |
| 357 | IMPI-061 | VL domain | GAAATTGTGTTGACGCAGTCTCCAGGCACCCTGTCTTTGTCTCCAGGGGAAAGAGCCACCCTCTCCTGCAGGGCCAG |
| | | nucleic | TCAGAGTGTTAGCGGCAGCCTCTTAGCCTGGTACCAGCAGAAACCTGGCCAGGCTCCCAGGCTCCTCATCTATGGTG |
| | | acid | CATCCAGAAGGGCCACTGGCATCCCAGACAGGTTCAGTGGCAGTGGGTCTGGGACAGACTTCACTCTCACCATCAGC |
| | | sequence | AGACTGGAGCCTGAAGATTTTGCAGTGTATTACTGTCAGCAATATGGTAACTCACCCATGTGCAGTTTTGGCCAGGG |
| | | | GACCAAGCTGGAGAGCAAA |
| 358 | IMPI-061 | VL domain | EIVLTQSPGTLSLSPGERATLSCRASQSVSGSLLAWYQQKPGQAPRLLIYGASRRATGIPDRFSGSGSGTDFTLTIS |
| | | amino acid | RLEPEDFAVYYCQQYGNSPMCSFGQGTKLESK |
| | | sequence | |
| 359 | IMPI-061 | LCDR1 | QSVSGSL |
| 157 | IMPI-061 | LCDR2 | GAS |
| 360 | IMPI-061 | LCDR3 | QQYGNSPMCS |
| 361 | IMPI-062 | VH domain | GAGGTGCAGCTGGTGGAGTCTGGGGGAGGTGTGGTACGGCCTGGGGGGTCCCTGAGACTCTCCTGTGCAGCCTCTGG |
| | | nucleic | ATTCACCTTTGATGATTATGGCATGAGCTGGGTCCGCCAAGCTCCAGGGAAGGGGCTGGAGTGGGTCTCTGGTATTT |
| | | acid | ATTGGAATGGTGGTAGTACAGGTTATGCAGACTCTGTGAAGGGCCGATTCACCATCTCCAGAGACAACGCCAAGAAC |
| | | sequence | TCCCTGTATCTGCAAATGAACAGTCTGAGAGCCGAGGACACGGCCTTGTATTACTGTGCGAGAGGAGTGGGAGCTAC |
| | | | AGACTACTGGGGCCAGGGAACCCTGGTCACCGTCTCCTCA |
| 353 | IMPI-062 | VH domain | EVQLVESGGGVVRPGGSLRLSCAASGFTFDDYGMSWVRQAPGKGLEWVSGIYWNGGSTGYADSVKGRFTISRDNAKN |
| | | amino acid | SLYLQMNSLRAEDTALYYCARGVGATDYWGQGTLVTVSS |
| | | sequence | |
| 354 | IMPI-062 | HCDR1 | GFTFDDYG |
| 355 | IMPI-062 | HCDR2 | IYWNGGST |
| 356 | IMPI-062 | HCDR3 | ARGVGATDY |
| 362 | IMPI-062 | VL domain | GAAATTGTGTTGACGCAGTCTCCAGGCACCCTGTCTTTGTCTCCAGGGGAAAGAGCCACCCTCTCCTGCAGGGCCAG |
| | | nucleic | TCAGAGTGTTAGAGGCAGCTTCTTAGCCTGGTACCAGCAGAAACCTGGCCAGGCTCCCAGGCTCCTCATCTATGGTG |
| | | acid | CATCCAGGAGGGCCACTGGCATCCCAGACAGGTTCAGTGGCAGTGGGTCTGGGACAGACTTCACTCTCACCATCAGC |
| | | sequence | AGACTGGAGCCTGAAGATTTTGCAGTGTATTACTGTCAGCAATATGGTAACTCACCCATGTGCAGTTTTGGCCAGGG |
| | | | GACCAAGCTGGAGATCAAA |
| 363 | IMPI-062 | VL domain | EIVLTQSPGTLSLSPGERATLSCRASQSVRGSFLAWYQQKPGQAPRLLIYGASRRATGIPDRFSGSGSGTDFTLTIS |
| | | amino acid | RLEPEDFAVYYCQQYGNSPMCSFGQGTKLEIK |
| | | sequence | |
| 364 | IMPI-062 | LCDR1 | QSVRGSF |
| 157 | IMPI-062 | LCDR2 | GAS |
| 360 | IMPI-062 | LCDR3 | QQYGNSPMCS |
| 365 | IMPI-063 | VH domain | GAGGTGCAGCTGGTGGAGTCTGGGGGAGGTGTGGTACGGCCTGGGGGGTCCCTGAGACTCTCCTGTGCAGCCTCTGG |
| | | nucleic | ATTCACCTTTGATGATTATGGCATGAGCTGGGTCCGCCAAGCTCCAGGGAAGGGGCTGGAGTGGGTCTCTGGTATTT |
| | | acid | ATTGGAATGGTGGTAGCACAGGTTATGCAGACTCTGTGAAGGGCCGATTCACCATCTCCAGAGACAACGCCAAGAAC |
| | | sequence | TCCCTGTATCTGCAAATGAACAGTCTGAGAGCCGAGGACATGGCCTTGTATTACTGTGCGAGAGGAGTGGGAGCTAC |
| | | | AGACTACTGGGGCCAGGGAACCCTGGTCACCGTCTCCTCA |
| 366 | IMPI-063 | VH domain | EVQLVESGGGVVRPGGSLRLSCAASGFTFDDYGMSWVRQAPGKGLEWVSGIYWNGGSTGYADSVKGRFTISRDNAKN |
| | | amino acid | SLYLQMNSLRAEDMALYYCARGVGATDYWGQGTLVTVSS |
| | | sequence | |
| 354 | IMPI-063 | HCDR1 | GFTFDDYG |
| 355 | IMPI-063 | HCDR2 | IYWNGGST |
| 356 | IMPI-063 | HCDR3 | ARGVGATDY |
| 367 | IMPI-063 | VL domain | GAAATTGTGTTGACGCAGTCTCCAGGCACCCTGTCTTTGTCTTCAGGGGAAAGAGCCACCCTCGCCTGCAGGGCCAG |
| | | nucleic | TGAGAGTGTTAGAGGCAGCTTCTTAGCCTGGTACCAGCAGAAACCTGGCCAGGCTCCCAGGCTCCTCATCTATGGTG |
| | | acid | CATCCAGGAGGGCCACTGGCATCCCAGACAGGTTCAGTGGCAGTGGGTCTGGGACAGACTTCACTCTCACCATCAGC |
| | | sequence | AGACTGGAGCCTGAGGATTTTGCAGTGTATTACTGTCAGCAATATGGTAATTCACCCATGTGCAGTTTTGGCCAGGG |
| | | | GACCAAGCTGGAGATCAAA |
| 368 | IMPI-063 | VL domain | EIVLTQSPGTLSLSSGERATLACRASESVRGSFLAWYQQKPGQAPRLLIYGASRRATGIPDRFSGSGSGTDFTLTIS |
| | | amino acid | RLEPEDFAVYYCQQYGNSPMCSFGQGTKLEIK |
| | | sequence | |
| 369 | IMPI-063 | LCDR1 | ESVRGSF |
| 157 | IMPI-063 | LCDR2 | GAS |
| 360 | IMPI-063 | LCDR3 | QQYGNSPMCS |
| 370 | IMPI-064 | VH domain | GAGGTGCAACTGGTGGAGTCTGGGGGAGGTGTGGTACGGCCTGGGGGGTCCCTGAGACTCTCCTGTGCAGCCTCTGG |
| | | nucleic | ATTCACCTTTGATGATTATGGCATGAGCTGGGTCCGCCAAGCTCCAGGGAAGGGGCTGGAGTGGGTCTCTGGTATTT |
| | | acid | ATTGGAATGGTGGTAGCACAGGTTATGCAGACTCTGTGAAGGGCCGATTCACCATCTCCAGAGACAACGCCAAGAAC |
| | | sequence | TCCCTGTATCTGCAAATGAACAGTCTGAGAGCCGAGGACACGGCCTTGTATTACTGTGCGAGAGGAGTGGGAGCTAC |
| | | | AGACTACTGGGGCCAGGGAACCCTGGTCACCGTCTCCTCA |
| 353 | IMPI-064 | VH domain | EVQLVESGGGVVRPGGSLRLSCAASGFTFDDYGMSWVRQAPGKGLEWVSGIYWNGGSTGYADSVKGRFTISRDNAKN |
| | | amino acid | SLYLQMNSLRAEDTALYYCARGVGATDYWGQGTLVTVSS |
| | | sequence | |
| 354 | IMPI-064 | HCDR1 | GFTFDDYG |
| 355 | IMPI-064 | HCDR2 | IYWNGGST |
| 356 | IMPI-064 | HCDR3 | ARGVGATDY |
| 371 | IMPI-064 | VL domain | GAAATTGTGTTGACGCAGTCTCCAGGCACCCTTTTTTTGTCTCCAGGGGAAAGAGCCACCCTCTCCTGCAGGGCCAG |
| | | nucleic | TCAGAGTGTTCGCGGCAGCTTCTTAGCCTGGTATCAACAGAAACCTGGCCAGGCTCCCAGGCTCCTCATCTATGGTG |
| | | acid | CATCCAGGAGGGCCACTGGCATCCCAGACAGGTTCAGTGGCAGTGGGTCTGGGACAGACTTCACTCTCACCATCAGT |
| | | sequence | AGACTGGAGCCTGAAGATTTTGCAGTGTATTACTGTCAGCAATATGGTCACTCACCCATGTGCAGTTTTGGCCAGGG |
| | | | GACCAAGCTGGAGATCAAA |
| 372 | IMPI-064 | VL domain | EIVLTQSPGTLFLSPGERATLSCRASQSVRGSFLAWYQQKPGQAPRLLIYGASRRATGIPDRFSGSGSGTDFTLTIS |
| | | amino acid | RLEPEDFAVYYCQQYGHSPMCSFGQGTKLEIK |
| | | sequence | |
| 364 | IMPI-064 | LCDR1 | QSVRGSF |
| 157 | IMPI-064 | LCDR2 | GAS |
| 373 | IMPI-064 | LCDR3 | QQYGHSPMCS |
| 352 | IMPI-065 | VH domain | GAGGTGCAGCTGGTGGAGTCTGGGGGAGGTGTGGTACGGCCTGGGGGGTCCCTGAGACTCTCCTGTGCAGCCTCTGG |
| | | nucleic | ATTCACCTTTGATGATTATGGCATGAGCTGGGTCCGCCAAGCTCCAGGGAAGGGGCTGGAGTGGGTCTCTGGTATTT |
| | | acid | ATTGGAATGGTGGTAGCACAGGTTATGCAGACTCTGTGAAGGGCCGATTCACCATCTCCAGAGACAACGCCAAGAAC |
| | | sequence | TCCCTGTATCTGCAAATGAACAGTCTGAGAGCCGAGGACACGGCCTTGTATTACTGTGCGAGAGGAGTGGGAGCTAC |
| | | | AGACTACTGGGGCCAGGGAACCCTGGTCACCGTCTCCTCA |
| 353 | IMPI-065 | VH domain | EVQLVESGGGVVRPGGSLRLSCAASGFTFDDYGMSWVRQAPGKGLEWVSGIYWNGGSTGYADSVKGRFTISRDNAKN |
| | | amino acid | SLYLQMNSLRAEDTALYYCARGVGATDYWGQGTLVTVSS |
| | | sequence | |
| 354 | IMPI-065 | HCDR1 | GFTFDDYG |
| 355 | IMPI-065 | HCDR2 | IYWNGGST |
| 356 | IMPI-065 | HCDR3 | ARGVGATDY |
| 374 | IMPI-065 | VL domain | GAAATTGTGTTGACGCAGTCTCCAGGCACCCTGTCTTTGTCTCCAGGGGAAAGAGCCACCCTCTCCTGTAGGGCCAG |
| | | nucleic | TCAGAGTGTTAGGGGCAGCTTCTTAGCCTGGTACCAGCAGAAACCTGGCCAGGCTCCCAGGCTCCTCATCTATGGTG |
| | | acid | CATCCAGCAGGGCCACTGGCATCCCAGACAGGTTCAGTGGCAGTGGGTCTGGGACAGACTTCACTCTCACCATCAGC |
| | | sequence | AGACTGGAGCCTGAAGATTTTGCAGTGTATTACTGTCAGCAATATGGTAAATCACCCATGTGCAGTTTTGGCCAGGG |
| | | | GACCAACCTGGAGATCAAA |
| 375 | IMPI-065 | VL domain | EIVLTQSPGTLSLSPGERATLSCRASQSVRGSFLAWYQQKPGQAPRLLIYGASSRATGIPDRFSGSGSGTDFTLTIS |
| | | amino acid | RLEPEDFAVYYCQQYGKSPMCSFGQGTNLEIK |
| | | sequence | |
| 364 | IMPI-065 | LCDR1 | QSVRGSF |
| 157 | IMPI-065 | LCDR2 | GAS |
| 376 | IMPI-065 | LCDR3 | QQYGKSPMCS |
| 377 | IMPI-066 | VH domain | GAGGTGCAGCTGGTGGAGTCTGGGGGAGGTGTGGTACGGCCTGGGGGGTCCCTGAGACTCTCCTGTGCAGCCTCTGG |
| | | nucleic | ATTCACCTTTGATGATTATGGCATGACCTGGGTCCGCCAAGCTCCAGGGAAGGGGCTGGAGTGGGTCTCTGGTATTA |
| | | acid | ATTGGAATGGAGGTAGCACAGGTTCTGCAGACTCTGTGAAGGGCCGATTCACCATCTCCAGAGACAACGCCAAGAAC |
| | | sequence | TCCCTGTGTCTGCAAATGAACAGTCTGAGAGCCGAGGACACGGCCTTGTATTACTGTGCGAGAGGAGTGGGAGCTAC |
| | | | AGATTACTGGGGCCAGGGAACCCTGGTCACCGTCTCCTCA |
| 378 | IMPI-066 | VH domain | EVQLVESGGGVVRPGGSLRLSCAASGFTFDDYGMTWVRQAPGKGLEWVSGINWNGGSTGSADSVKGRFTISRDNAKN |
| | | amino acid | SLCLQMNSLRAEDTALYYCARGVGATDYWGQGTLVTVSS |
| | | sequence | |
| 354 | IMPI-066 | HCDR1 | GFTFDDYG |
| 379 | IMPI-066 | HCDR2 | INWNGGST |
| 356 | IMPI-066 | HCDR3 | ARGVGATDY |
| 380 | IMPI-066 | VL domain | GAAATTGTGTTGACGCAGTCTCCAGGCACCCTGTCTTTGTCTCCAGGGGAAAGAGCCACCCTCTCCTGCAGGGCCAG |
| | | nucleic | TCAGAGTGTTAGCAGCAGCTTCTTAGCCTGGTACCAGCAAAAACCTGGCCAGGCTCCCAGGCTCCTCATCTATGGTG |
| | | acid | CATCCCGCAGGTCCACTGGCATCCCAGACAGGTTCAGTGGCAGTGGGTCTGGGACAGACTTCACTCTCACCATCAGC |
| | | sequence | AGACTGGAGCCTGAAGATTTTGCAGTGTATTACTGTCAGCAGTATGGTAGCTCACCCATGTCCAGTTTTGGCCAGGG |
| | | | GACCAAGCTGGAGATCAAA |
| 381 | IMPI-066 | VL domain | EIVLTQSPGTLSLSPGERATLSCRASQSVSSSFLAWYQQKPGQAPRLLIYGASRRSTGIPDRFSGSGSGTDFTLTIS |
| | | amino acid | RLEPEDFAVYYCQQYGSSPMSSFGQGTKLEIK |
| | | sequence | |
| 382 | IMPI-066 | LCDR1 | QSVSSSF |
| 157 | IMPI-066 | LCDR2 | GAS |
| 383 | IMPI-066 | LCDR3 | QQYGSSPMSS |
| 384 | IMPI-067 | VH domain | CAGGTGCAGCTGGTGGAGTCTGGGGGAGGCGTGGTCCAGCCTGGGACGTCCCTGAGACTCTCCTGTGCAGCCTCTGG |
| | | nucleic | ATTCACCTTCAATAGCTTTGGCATGCACTGGGTCCGCCAGGCTCCAGGCAAGGGGCTGGAGTGGGTGGCAGTTATAT |
| | | acid | CATATGATGGAAGTAGTAAATACTATGCAGACTCCGTGAAGGGCCGATTCACCATCTCCAGAGACAATTCCAAGAAC |
| | | sequence | ACGCTGTATCTGCAAATGAACAGCCTGAGAGCGGAGGACACGGCTGTGTATTACTGTGCGAAAGATGAGGGGGACTA |
| | | | CGACGACTACTACTACGGTATGGACGTCTGGGGCCAGGGGACCACGGTCACCGTCTCCTCA |
| 385 | IMPI-067 | VH domain | QVQLVESGGGVVQPGTSLRLSCAASGFTFNSFGMHWVRQAPGKGLEWVAVISYDGSSKYYADSVKGRFTISRDNSKN |
| | | amino acid | TLYLQMNSLRAEDTAVYYCAKDEGDYDDYYYGMDVWGQGTTVTVSS |
| | | sequence | |
| 386 | IMPI-067 | HCDR1 | GFTFNSFG |
| 387 | IMPI-067 | HCDR2 | ISYDGSSK |
| 388 | IMPI-067 | HCDR3 | AKDEGDYDDYYYGMDV |
| 389 | IMPI-067 | VL domain | GAAATTGTGTTGACGCAGTCTCCAGGCAGTCTGTCTTTGTCTCCAGGGGAAAGAGCCACCCTCTCCTGCAGGGCCAG |
| | | nucleic | TCAGAGTCTTAGCAGCAGATACTTGGCCTGGTACCACCAGAAACCTGGCCAGGCTCCCAGGCTCCTCATCTATGGTG |
| | | acid | CATCCAGCAGGGCCACTGGCATCCCAGACAGGTTCAGTGGCAGTGGGTCTGGGACAGACTTCACTCTCACCATCAGC |
| | | sequence | GGACTGGAGCCTGAAGATTTTGCAGTGTATTACTGTCAGCAGTATGGCAACTCAATCACTTTCGGCGGAGGGACCAA |
| | | | GGTGGAAATCAAA |
| 390 | IMPI-067 | VL domain | EIVLTQSPGSLSLSPGERATLSCRASQSLSSRYLAWYHQKPGQAPRLLIYGASSRATGIPDRFSGSGSGTDFTLTIS |
| | | amino acid | GLEPEDFAVYYCQQYGNSITFGGGTKVEIK |
| | | sequence | |
| 391 | IMPI-067 | LCDR1 | QSLSSRY |
| 157 | IMPI-067 | LCDR2 | GAS |
| 392 | IMPI-067 | LCDR3 | QQYGNSIT |
| 393 | IMPI-068 | VH domain | CAGGTGCAGCTGGTGGAGTCTGGGGGAGACGTGGTCCAGCCTGGGAGGTCCCTGAGACTCTCCTGTGCAGCCTCTGG |
| | | nucleic | ATTCACCTTCAGTCGCTATGGCATGCACTGGGTCCGCCAGGCTCCAGGCAAGGGGCTGGAGTGGGTGGCAGTTATAT |
| | | acid | CATATGATGGAAGTGATAAATACTATGCAGACTCCGTGAAGGGCCGATTCACCATCTCCAGAGACAATTCCAAGAAC |
| | | sequence | ACGCTGTATCTGCAAATGAACAGCCTGAGAGCTGAGGACACGGCTGTGTATTACTGTGCGAAACAGCTGCCCCTTTA |
| | | | CTACTACTACTACGGTATGGACGTCTGGGGCCAAGGGACCACGGTCACCGTCTCCTCA |
| 394 | IMPI-068 | VH domain | QVQLVESGGDVVQPGRSLRLSCAASGFTFSRYGMHWVRQAPGKGLEWVAVISYDGSDKYYADSVKGRFTISRDNSKN |
| | | amino acid | TLYLQMNSLRAEDTAVYYCAKQLPLYYYYYGMDVWGQGTTVTVSS |
| | | sequence | |
| 395 | IMPI-068 | HCDR1 | GFTFSRYG |
| 113 | IMPI-068 | HCDR2 | ISYDGSDK |
| 396 | IMPI-068 | HCDR3 | AKQLPLYYYYYGMDV |
| 397 | IMPI-068 | VL domain | GCCATCCAGTTGACCCAGTCTCCATCCTCCCTGTCTGCATCTGTAGGAGACAGAGTCACCATCACTTGCCGGGCAAG |
| | | nucleic | TCAGGGCATTAGCAGTGCTTTAGCCTGGTATCAGCAGAAACCAGGGAAAGCTCCTAACCTCCTAATCTATGATGCCT |
| | | acid | CCAGTTTGGAAAGTGGGGTCCCATCAAGGTTCAGCGGCAGTGGATCTGGGACAGATTTCACTCTCACCATCAGCAGC |
| | | sequence | CTGCAGCCTGAAGATTTTGCAACTTATTACTGTCAACAGTTTAATATTTACCCTCACACTTTCGGCCCTGGGACCAA |
| | | | AGTGGATATCAAA |
| 398 | IMPI-068 | VL domain | AIQLTQSPSSLSASVGDRVTITCRASQGISSALAWYQQKPGKAPNLLIYDASSLESGVPSRFSGSGSGTDFTLTISS |
| | | amino acid | LQPEDFATYYCQQFNIYPHTFGPGTKVDIK |
| | | sequence | |
| 37 | IMPI-068 | LCDR1 | QGISSA |
| 28 | IMPI-068 | LCDR2 | DAS |
| 399 | IMPI-068 | LCDR3 | QQFNIYPHT |
| 400 | IMPI-069 | VH domain | GAGGTGCAACTGGTGGAGTCTGGGGGAGGTGTGGTACGGCCTGGGGGGTCCCTGAGACTCTCCTGTGCAGCCTCTGG |
| | | nucleic | ATTCACCTTTGATGATTATGGCATGACCTGGGTCCGCCAAGCTCCAGGGAAGGGGCTGGAGTGGGTCTCTGGTATTT |
| | | acid | ATTGGAATGGTGGTAGCACAGGTTATGCAGACTCTGTGAAGGGCCGATTCACCATCTCCAGAGACAACGCCAAGAAC |
| | | sequence | TCCCTGTATCTGCAAATGAACAGTCTGAGAGCCGAGGACACGGCCTTGTATTACTGTGCGAGAGGAGTGGGAGCTAC |
| | | | AGACTACTGGGGCCAGGGAACCCTGGTCACCGTCTCCTCA |
| 401 | IMPI-069 | VH domain | EVQLVESGGGVVRPGGSLRLSCAASGFTFDDYGMTWVRQAPGKGLEWVSGIYWNGGSTGYADSVKGRFTISRDNAKN |
| | | amino acid | SLYLQMNSLRAEDTALYYCARGVGATDYWGQGTLVTVSS |
| | | sequence | |
| 354 | IMPI-069 | HCDR1 | GFTFDDYG |
| 355 | IMPI-069 | HCDR2 | IYWNGGST |
| 356 | IMPI-069 | HCDR3 | ARGVGATDY |
| 402 | IMPI-069 | VL domain | GAAATTGTGTTGACGCAGTCTCCAGGCACCCTGTTTTTGTCTCCAGGGGAAAGAGCCACCCTCTCCTGCAGGACCAG |
| | | nucleic | TCAGAATATTAGCGGCAGCTTCTTAGCCTGGTACCAGCAGAAATCTGGCCAGGTTCCCAGGCTCCTCATCTATGGTG |
| | | acid | CATCCAGGAGGGCCACTGGCATCCCAGACAGGTTCAGTGGCAGTGGGTCTGGGACAGACTTCACTCTCACCATCAGC |
| | | sequence | AGACTGGAGCCTGAAGATTTTGCAGTGTATTACTGTCAGCAATATGGTAACTCACCCATGTGCAGTTTTGGCCAGGG |
| | | | GACCAAGCTGGAGATCAAA |
| 403 | IMPI-069 | VL domain | EIVLTQSPGTLFLSPGERATLSCRTSQNISGSFLAWYQQKSGQVPRLLIYGASRRATGIPDRFSGSGSGTDFTLTIS |
| | | amino acid | RLEPEDFAVYYCQQYGNSPMCSFGQGTKLEIK |
| | | sequence | |
| 404 | IMPI-069 | LCDR1 | QNISGSF |
| 157 | IMPI-069 | LCDR2 | GAS |
| 360 | IMPI-069 | LCDR3 | QQYGNSPMCS |
| 370 | IMPI-070 | VH domain | GAGGTGCAACTGGTGGAGTCTGGGGGAGGTGTGGTACGGCCTGGGGGGTCCCTGAGACTCTCCTGTGCAGCCTCTGG |
| | | nucleic | ATTCACCTTTGATGATTATGGCATGAGCTGGGTCCGCCAAGCTCCAGGGAAGGGGCTGGAGTGGGTCTCTGGTATTT |
| | | acid | ATTGGAATGGTGGTAGCACAGGTTATGCAGACTCTGTGAAGGGCCGATTCACCATCTCCAGAGACAACGCCAAGAAC |
| | | sequence | TCCCTGTATCTGCAAATGAACAGTCTGAGAGCCGAGGACACGGCCTTGTATTACTGTGCGAGAGGAGTGGGAGCTAC |
| | | | AGACTACTGGGGCCAGGGAACCCTGGTCACCGTCTCCTCA |
| 353 | IMPI-070 | VH domain | EVQLVESGGGVVRPGGSLRLSCAASGFTFDDYGMSWVRQAPGKGLEWVSGIYWNGGSTGYADSVKGRFTISRDNAKN |
| | | amino acid | SLYLQMNSLRAEDTALYYCARGVGATDYWGQGTLVTVSS |
| | | sequence | |
| 354 | IMPI-070 | HCDR1 | GFTFDDYG |
| 355 | IMPI-070 | HCDR2 | IYWNGGST |
| 356 | IMPI-070 | HCDR3 | ARGVGATDY |
| 405 | IMPI-070 | VL domain | GAAAATGTGTTGACGCAGTCTCCAGGCACCCTGTTTTTGTCTCCAGGGGAAAGAGCCACCCTCTCCTGCAGGGCCAG |
| | | nucleic | TCAGAGTGTTAGCGGCAGCTTCTTAGCCTGGTACCAGCAGAAACCTGGCCAGGCTCCCAGGCTCCTCATTTATGGTG |
| | | acid | CATCCAGGAGGGCCACTGGCATCCCAGACAGGTTCAGTGGCAGTGGGGCTGGGACAGACTTCACTCTCACCATCAGC |
| | | sequence | AGACTGGAGCCTGAAGATTTTGCAGTGTATTACTGTCAGCAATATAATAACTCACCCATGTGCAGTTTTGGCCAGGG |
| | | | GACCAAGCTGGAGATCAAA |
| 406 | IMPI-070 | VL domain | ENVLTQSPGTLFLSPGERATLSCRASQSVSGSFLAWYQQKPGQAPRLLIYGASRRATGIPDRFSGSGAGTDFTLTIS |
| | | amino acid | RLEPEDFAVYYCQQYNNSPMCSFGQGTKLEIK |
| | | sequence | |
| 407 | IMPI-070 | LCDR1 | QSVSGSF |
| 157 | IMPI-070 | LCDR2 | GAS |
| 408 | IMPI-070 | LCDR3 | QQYNNSPMCS |
| 370 | IMPI-071 | VH domain | GAGGTGCAACTGGTGGAGTCTGGGGGAGGTGTGGTACGGCCTGGGGGGTCCCTGAGACTCTCCTGTGCAGCCTCTGG |
| | | nucleic | ATTCACCTTTGATGATTATGGCATGAGCTGGGTCCGCCAAGCTCCAGGGAAGGGGCTGGAGTGGGTCTCTGGTATTT |
| | | acid | ATTGGAATGGTGGTAGCACAGGTTATGCAGACTCTGTGAAGGGCCGATTCACCATCTCCAGAGACAACGCCAAGAAC |
| | | sequence | TCCCTGTATCTGCAAATGAACAGTCTGAGAGCCGAGGACACGGCCTTGTATTACTGTGCGAGAGGAGTGGGAGCTAC |
| | | | AGACTACTGGGGCCAGGGAACCCTGGTCACCGTCTCCTCA |
| 353 | IMPI-071 | VH domain | EVQLVESGGGVVRPGGSLRLSCAASGFTFDDYGMSWVRQAPGKGLEWVSGIYWNGGSTGYADSVKGRFTISRDNAKN |
| | | amino acid | SLYLQMNSLRAEDTALYYCARGVGATDYWGQGTLVTVSS |
| | | sequence | |
| 354 | IMPI-071 | HCDR1 | GFTFDDYG |
| 355 | IMPI-071 | HCDR2 | IYWNGGST |
| 356 | IMPI-071 | HCDR3 | ARGVGATDY |
| 409 | IMPI-071 | VL domain | GAAATTGTGTTGACGCAGTCTCCAGGCACCCTGTTTTTGTCTCCAGGGGAAAGAGCCACCCTCTCCTGCAGGGCCAG |
| | | nucleic | TCAGAGTGTTCGCGGCAGCTTCTTAGCCTGGTATCAGCAGAAACCTGGCCAGGCTCCCAGGCTCCTCATCAATGGTG |
| | | acid | CATCCAGGAGGGCCACTGGCATCCCAGACAGGTTCAGTGGCAGTGGGTCTGGGACAGACTTCACTCTCACCATCAGT |
| | | sequence | AGACTGGAGCCTGAAGATTTTGCAGTGTATTACTGTCAGCAATATGGTAACTCACCCATGTGCAGTTTTGGCCAGGG |
| | | | GACCAAGCTGGAGATCAAA |
| 410 | IMPI-071 | VL domain | EIVLTQSPGTLFLSPGERATLSCRASQSVRGSFLAWYQQKPGQAPRLLINGASRRATGIPDRFSGSGSGTDFTLTIS |
| | | amino acid | RLEPEDFAVYYCQQYGNSPMCSFGQGTKLEIK |
| | | sequence | |
| 364 | IMPI-071 | LCDR1 | QSVRGSF |
| 157 | IMPI-071 | LCDR2 | GAS |
| 360 | IMPI-071 | LCDR3 | QQYGNSPMCS |
| 411 | IMPI-072 | VH domain | CAGGTGCAACTGGTGGAGTCTGGGGGAGGCGTGGTCCAGCCTGGGAGGTCCCTGAGACTCTCCTGTGCAGCCTCTGG |
| | | nucleic | ATTCACCTTCAGTAGCTTTGGCATGCACTGGGTCCGCCAGGCTCCAGGCAAGGGGCTGGAGTGGGTGGCAGTTATAT |
| | | acid | CATATGATGGAAGTAGTAAAGACTATGCAGACTCCGTGAAGGGCCGATTCACCATCTCCAGAGACAATTCCAAGAAC |
| | | sequence | ACGCTGTATCTGCAAATGAACAGCCTGGGAGCGGAGGACACGGCTGTGTATTACTGTGCGAAAGATGAGGGGGACTA |
| | | | CGACGACTACTATTACGGTATGGACGTCTGGGGCCAAGGGACCACGGTCACCGTCTCCTCA |
| 412 | IMPI-072 | VH domain | QVQLVESGGGVVQPGRSLRLSCAASGFTFSSFGMHWVRQAPGKGLEWVAVISYDGSSKDYADSVKGRFTISRDNSKN |
| | | amino acid | TLYLQMNSLGAEDTAVYYCAKDEGDYDDYYYGMDVWGQGTTVTVSS |
| | | sequence | |
| 413 | IMPI-072 | HCDR1 | GFTFSSFG |
| 387 | IMPI-072 | HCDR2 | ISYDGSSK |
| 388 | IMPI-072 | HCDR3 | AKDEGDYDDYYYGMDV |
| 414 | IMPI-072 | VL domain | GAAATTGTGTTGACGCAGTCTCCAGGCACCCTGTCTTTGTCTCCAGGGGAAAGAGCCACCCTCTCCTGCAGGGCCAG |
| | | nucleic | TCAGAGTCTTAGCAGCAGATACTTGGCCTGGTACCACCAGAAACCTGGCCAGGCTCCCAGGCTCTTCATCTATGGTG |
| | | acid | CATCCATCAGGGCCACTGGCATCCCAGACAGGTTCAGTGGCAGTGGGTCTGGGACGGACTTCACTCTCACCATCAGC |
| | | sequence | GGACTGGAGCCTGAAGATTTTGCAGTGTATTACTGTCAGCAGTATGGTAGTTCAATCACTTTCGGCAGAGGGACCAA |
| | | | GGTGGAGATCAAA |
| 415 | IMPI-072 | VL domain | EIVLTQSPGTLSLSPGERATLSCRASQSLSSRYLAWYHQKPGQAPRLFIYGASIRATGIPDRFSGSGSGTDFTLTIS |
| | | amino acid | GLEPEDFAVYYCQQYGSSITFGRGTKVEIK |
| | | sequence | |
| 391 | IMPI-072 | LCDR1 | QSLSSRY |
| 157 | IMPI-072 | LCDR2 | GAS |
| 416 | IMPI-072 | LCDR3 | QQYGSSIT |

| SEQ ID | Clone | | |
|---|---|---|---|
| NO | ID | Description | Sequence |
| 486 | YANG-1101 | VH domain | CAGCTGCAGCTGCAGGAGTCGGGCCCAGGACTGGTGAAGCCTTC |
| | | nucleic | GGAGACCCTGTCCCTCACCTGCACTGTCTCTGGTGGCTCCATCA |
| | | acid | GCAGTAGTAGTTACTACTGGGGCTGGATCCGCCAGCCCCCAGGG |
| | | sequence | AAGGGGCTGGAGTGGATTGGGAGTATCTATTATAGTGGGAGCAT |
| | | | TTACTACAACCCGTCCCTCAAGAGTCGAGTCACCATATCCGTTG |
| | | | ACACGTCCAAGAACCAGTTCTCCCTGAAGCTGAGCTCTGTGACC |
| | | | GCCGCAGACACGGCTGTGTATTACTGTGCGAGCTGGTTCGGGGA |
| | | | GTTCTTGAAAGCCGATGCTTTTGATATCTGGGGCCAAGGGACAA |
| | | | TGGTCACCGTCTCTTCA |
| 487 | YANG-1101 | VH domain | QLQLQESGPGLVKPSETLSLTCTVSGGSISSSSYYWGWIRQPPG |
| | | amino | KGLEWIGSIYYSGSIYYNPSLKSRVTISVDTSKNQFSLKLSSVT |
| | | acid | AADTAVYYCASWFGEFLKADAFDIWGQGTMVTVSS |
| | | sequence | |
| 488 | YANG-1101 | HCDR1 | GGSISSSSYY |
| 489 | YANG-1101 | HCDR2 | IYYSGSI |
| 490 | YANG-1101 | HCDR3 | ASWFGEFLKADAFDI |
| 491 | YANG-1101 | VL domain | CAGTCTGCCCTGACTCAGCCTGCCTCCGTGTCTGGGTCTCCTGG |
| | | nucleic | ACAGTCGATCACCATCTCCTGCACTGGAACCAGCAGTGATGTTG |
| | | acid | GTGGTTATAGCTATGTCTCCTGGTACCAACAGCACCCAGGCAAA |
| | | sequence | GCCCCCAAACTCATGATTTATGATGTCAGTAAGCGGCCCTCAGG |
| | | | GGTTTCTAATCGCTTCTCTGGCTCCAAGTCTGGCAACACGGCCT |
| | | | CCCTGACAATCTCTGGGCTCCAGGCTGAGGACGAGGCTGATTAT |
| | | | TACTGCTGCTCATATGCAGGTGGTAGCACTTTCGTGGTATTCGG |
| | | | CGGAGGGACCAAGCTGACCGTCCTA |
| 492 | YANG-1101 | VL domain | QSALTQPASVSGSPGQSITISCTGTSSDVGGYSYVSWYQQHPGK |
| | | amino | APKLMIYDVSKRPSGVSNRFSGSKSGNTASLTISGLQAEDEADY |
| | | acid | YCCSYAGGSTFVVFGGGTKLTVL |
| | | sequence | |
| 493 | YANG-1101 | LCDR1 | SSDVGGYSY |
| 203 | YANG-1101 | LCDR2 | DVS |
| 494 | YANG-1101 | LCDR3 | CSYAGGSTFVV |
| 495 | YANG-1102 | VH domain | CAGGTGCAGCTGCAGGAGTCGGGCCCAGGACTGGTGAAGCCTTC |
| | | nucleic | GGGGACCCTGTCCCTCACCTGCGCTGTCTCTGGTGGCTCCATCA |
| | | acid | GCACAACTAATTGGTGGAGTTGGGTCCGCCAGTCCCCAGGGAGG |
| | | sequence | GGGCTGGAGTGGATTGGGGAAATCTTTCATATTGGATATACCAA |
| | | | CTACAACCCGTCCCTCAAGAGTCGAGTCACCATATCAGTAGACA |
| | | | AGTCCAAGAACCAATTCTCCCTGAAGCTGAATTCCGTGACCGTC |
| | | | GCGGACACGGCCGTGTATTACTGTGCGAGAAGAGTGGTCATGGA |
| | | | CACAGCTATGGCCCACTTTGACTGCTGGGGCCAGGGAACCCTGG |
| | | | TCACCGTCTCCTCA |
| 496 | YANG-1102 | VH domain | QVQLQESGPGLVKPSGTLSLTCAVSGGSISTTNWWSWVRQSPGR |
| | | amino | GLEWIGEIFHIGYTNYNPSLKSRVTISVDKSKNQFSLKLNSVTV |
| | | acid | ADTAVYYCARRVVMDTAMAHFDCWGQGTLVTVSS |
| | | sequence | |
| 497 | YANG-1102 | HCDR1 | GGSISTTNW |
| 498 | YANG-1102 | HCDR2 | IFHIGYT |
| 499 | YANG-1102 | HCDR3 | ARRVVMDTAMAHFDC |
| 500 | YANG-1102 | VL domain | CAGTCTGTGCTGACGCAGCCGCCCTCAGTGTCTGGGGCCCCAGG |
| | | nucleic | GCAGAGGGTCACCATCTCCTGCACTGGGAGCAGCTCCAACATCG |
| | | acid | GGGCAGGTTATGATGTACACTGGTACCAGCACCTTCCAGGAACA |
| | | sequence | GCCCCCAAACTCCTCATCTATATTAACATCAATCGGCCCTCAGG |
| | | | GGTCCCTGACCGATTCTCTGGCTCCAAGTCTGGCACCTCAGCCT |
| | | | CCCTGGTCATCGCTGGGCTCCAGGCTGAAGATGGGGCTGATTAT |
| | | | TACTGCCAGTCCTATGACAGCAGTCTGAGTGGTTGGGTGTTCGG |
| | | | CGGGGGGACCAGGCTGACCGTCCTA |
| 501 | YANG-1102 | VL domain | QSVLTQPPSVSGAPGQRVTISCTGSSSNIGAGYDVHWYQHLPGT |
| | | amino | APKLLIYININRPSGVPDRFSGSKSGTSASLVIAGLQAEDGADY |
| | | acid | YCQSYDSSLSGWVFGGGTRLTVL |
| | | sequence | |
| 502 | YANG-1102 | LCDR1 | SSNIGAGYD |
| 503 | YANG-1102 | LCDR2 | INI |
| 504 | YANG-1102 | LCDR3 | QSYDSSLSGWV |
| 505 | YANG-1103 | VH domain | GAGGTGCAGATGGTGGAGTCTGGAGGAGGCTTGATCCAGCCGGG |
| | | nucleic | GGGGTCCCTGAGACTCTCCTGTGCAGCCTCTGGGCTCACCGTCA |
| | | acid | GTAGTAATTACATGAGCTGGGTCCGCCAGGCTCCAGGGAAGGGG |
| | | sequence | CTGGAGTGGGTCTCAGTTATTTATAGCGGTGGTAGCACATTCTA |
| | | | CGCAGACTCCGTGAAGGACCGATTCACCATCTCCAGGGACAATT |
| | | | CCAAGAACACGCTGTATCTTCAAATGAACAGCCTGAGAGCCGAA |
| | | | GACACGGCCGTATATTACTGTGCGCGAGAAAACTGGAACTACGT |
| | | | TTTTGACTGCTGGGGCCAGGGAACCCTGGTCACCGTCTCCTCA |
| 506 | YANG-1103 | VH domain | EVQMVESGGGLIQPGGSLRLSCAASGLTVSSNYMSWVRQAPGKG |
| | | amino | LEWVSVIYSGGSTFYADSVKDRFTISRDNSKNTLYLQMNSLRAE |
| | | acid | DTAVYYCARENWNYVEDCWGQGTLVTVSS |
| | | sequence | |
| 172 | YANG-1103 | HCDR1 | GLTVSSNY |
| 14 | YANG-1103 | HCDR2 | IYSGGST |
| 507 | YANG-1103 | HCDR3 | ARENWNYVEDC |
| 508 | YANG-1103 | VL domain | GACATCCAGTTGACCCAGTCTCCATCCTTCCTGTCTGCATCTGT |
| | | nucleic | AGGAGACAGAGTCACCATCACTTGCTGGGCCAGTCAGGGCATTA |
| | | acid | GCAGTTATTTAGCCTGGTATCAGCAAAAACCAGGGAAAGCCCCT |
| | | sequence | AAGCTCCTGATCTATGCTGCATCCACTTTGCAAAGTGGGGTCCC |
| | | | ATCAAGGTTCAGCGGCAGTGGATCTGGGACAGAATTCACTCTCA |
| | | | CAATCAGCAGCCTGCAGCCTGAAGATTTTGCAACTTATTACTGT |
| | | | CAACAGCTTAATAGTCACCCTATCATCTTCGGCCAAGGGACACG |
| | | | ACTGGAGATTAAA |
| 509 | YANG-1103 | VL domain | DIQLTQSPSFLSASVGDRVTITCWASQGISSYLAWYQQKPGKAP |
| | | amino | KLLIYAASTLQSGVPSRFSGSGSGTEFTLTISSLQPEDFATYYC |
| | | acid | QQLNSHPIIFGQGTRLEIK |
| | | sequence | |
| 8 | YANG-1103 | LCDR1 | QGISSY |
| 18 | YANG-1103 | LCDR2 | AAS |
| 510 | YANG-1103 | LCDR3 | QQLNSHPII |
| 511 | YANG-1105 | VH domain | GAGGTGCAGGTGGTAGAATCTGGAGGAGGCTTGATCCAGCCGGG |
| | | nucleic | GGGGTCCCTGAGACTCTCCTGTGCAGCCTCTGGAATCACCGTCA |
| | | acid | GTAGAAACTACATGAACTGGGTCCGCCAGGCTCCAGGGAAGGGG |
| | | sequence | CTGGAGTGGGTCTCAGTTATTTATAGCGGTGGTAGCACATTCTA |
| | | | CGCAGACTCCGTGAAGGGCCGATTCACCATCTCCAGAGACAATT |
| | | | CCAAGAACACGCTGTATCTTCAAATGAACAGCCTGAGAGCCGAT |
| | | | GACACGGGCGTGTATTACTGTGCGAGAGAAGGGTACGGTATGGA |
| | | | CGTCTGGGGCCAAGGGACCACGGTCACCGTCTCCTCA |
| 512 | YANG-1105 | VH domain | EVQVVESGGGLIQPGGSLRLSCAASGITVSRNYMNWVRQAPGKG |
| | | amino | LEWVSVIYSGGSTFYADSVKGRFTISRDNSKNTLYLQMNSLRAD |
| | | acid | DTGVYYCAREGYGMDVWGQGTTVTVSS |
| | | sequence | |
| 513 | YANG-1105 | HCDR1 | GITVSRNY |
| 14 | YANG-1105 | HCDR2 | IYSGGST |
| 514 | YANG-1105 | HCDR3 | AREGYGMDV |
| 515 | YANG-1105 | VL domain | GACATCCAGTTGACCCAGTCTCCATCCTTCCTGTCTGCATCTGT |
| | | nucleic | AGGAGACAGAGTCACCATCACTTGCTGGGCCAGTCAGGGCATTA |
| | | acid | GCAGTTATTTAGCCTGGTATCAGCAAAAACCAGGGAAAGCCCCT |
| | | sequence | AAGCTCCTGATTTATTCTGCATCCACTTTGCAAAGTGGGGTCCC |
| | | | ATCAAGGTTCAGCGGCAGTGGATCTGGGACAGAATTCACTCTCA |
| | | | CAATCAACAGCCTGCAGCCTGAAGATTTTGCAACTTATTATTGT |
| | | | CAACAGCTTAATAGTCACCCGTGCAGTTTTGGCCAGGGGACCAA |
| | | | GCTGGAGATCAAA |
| 516 | YANG-1105 | VL domain | DIQLTQSPSFLSASVGDRVTITCWASQGISSYLAWYQQKPGKAP |
| | | amino | KLLIYSASTLQSGVPSRFSGSGSGTEFTLTINSLQPEDFATYYC |
| | | acid | QQLNSHPCSFGQGTKLEIK |
| | | sequence | |
| 8 | YANG-1105 | LCDR1 | QGISSY |
| 517 | YANG-1105 | LCDR2 | SAS |
| 518 | YANG-1105 | LCDR3 | QQLNSHPCS |
| 519 | YANG-1106 | VH domain | GAGGTGCAGCTGGTGGAGTCTGGAGGAGGCCTGATCCAGCCTGG |
| | | nucleic | GGGGTCCCTGAGACTCTCCTGTGCAGCCTCTGAATTCATCGTCA |
| | | acid | GTCGCAACTACATGAGTTGGGTCCGCCAGGCTCCAGGGAAGGGG |
| | | sequence | CTGGAATGGGTCTCAGTTATTTATAGCGGTGGTAGCACATTCTA |
| | | | CGCAGACTCCGTGAGGGGCCGATTCACCATCTCCAGAGACAATT |
| | | | CCAAAAACACGCTGTATCTTCAAATGAACAGCCTGAGAGCCGAG |
| | | | GACACGGCCGTGTATTACTGTGCGAGAGACTACGGTGACCAGTA |
| | | | CTTTGACTACTGGGGCCAGGGAACCCTGGTCACCGTCTCCTCA |
| 520 | YANG-1106 | VH domain | EVQLVESGGGLIQPGGSLRLSCAASEFIVSRNYMSWVRQAPGKG |
| | | amino | LEWVSVIYSGGSTFYADSVRGRFTISRDNSKNTLYLQMNSLRAE |
| | | acid | DTAVYYCARDYGDQYFDYWGQGTLVTVSS |
| | | sequence | |
| 521 | YANG-1106 | HCDR1 | EFIVSRNY |
| 14 | YANG-1106 | HCDR2 | IYSGGST |
| 522 | YANG-1106 | HCDR3 | ARDYGDQYFDY |
| 523 | YANG-1106 | VL domain | GAAATGGTGATGACGCAGTCTCCAGCCACCCTGTCTGTGTCTCC |
| | | nucleic | AGGGGAAAAAGCCACCCTCTCCTGCAGGGCCAGTCAGACTGTTA |
| | | acid | GCACCAACTTAGCCTGGTACCAACAGAAACCTGGCCAGTCTCCC |
| | | sequence | AGGCTCCTCATCTATGGTGCATCCACCAGGGCCACTGGTATCCC |
| | | | AGCCAGGTTCAGTGGCAGTGGGTCTGGGACAGAGTTCACTCTCA |
| | | | CCATCAGCAGCCTGCAGTCTGAAGATTTTGCAATTTATTACTGT |
| | | | CAGCAATATGTTGACTGGCCTCGGACGTTCGGCCAAGGGACCAC |
| | | | GGTGGAAATCAAA |
| 524 | YANG-1106 | VL domain | EMVMTQSPATLSVSPGEKATLSCRASQTVSTNLAWYQQKPGQSP |
| | | amino | RLLIYGASTRATGIPARFSGSGSGTEFTLTISSLQSEDFAIYYC |
| | | acid | QQYVDWPRTFGQGTTVEIK |
| | | sequence | |
| 525 | YANG-1106 | LCDR1 | QTVSTN |
| 157 | YANG-1106 | LCDR2 | GAS |
| 526 | YANG-1106 | LCDR3 | QQYVDWPRT |
| 527 | YANG-1107 | VH domain | GAGGTGCAGATGGTGGAGTCTGGAGGAGGCTTGATCCAGTCTGG |
| | | nucleic | GGGGTCCCTGAGACTCTCCTGTGCAGCCTCTGGGCTCAGCGTCA |
| | | acid | GTAGCAACTACATGAGTTGGGTCCGCCAGGCTCCAGGGAAGGGG |
| | | sequence | CTGGAATGGGTCGCAGTTATTTATAGCGGTGGTAGTATATTCTA |
| | | | TGCAGACTCCGTGAAGGACCGATTCACCATCTCCAGGGACAATT |
| | | | CCAAGAACACGCTTTATCTTCAAATGAACAGCCTGAGAGCCGAG |
| | | | GACACGGCCGTATATTACTGTGCGAGAGAGAACTGGAACTACGT |
| | | | TTTTGACTGCTGGGGCCAGGGAACCCTGGTCACCGTCTCCTCA |
| 528 | YANG-1107 | VH domain | EVQMVESGGGLIQSGGSLRLSCAASGLSVSSNYMSWVRQAPGKG |
| | | amino | LEWVAVIYSGGSIFYADSVKDRETISRDNSKNTLYLQMNSLRAE |
| | | acid | DTAVYYCARENWNYVFDCWGQGTLVTVSS |
| | | sequence | |
| 529 | YANG-1107 | HCDR1 | GLSVSSNY |
| 530 | YANG-1107 | HCDR2 | IYSGGSI |
| 507 | YANG-1107 | HCDR3 | ARENWNYVFDC |
| 531 | YANG-1107 | VL domain | GACATCCAGTTGACCCAGTCTCCATCCTTCCTGTCTGCATCTGT |
| | | nucleic | TGGAGACAGAGTCACCATCACTTGCTGGGCCAGTCAGGGCATTA |
| | | acid | GCACTTATTTAGCCTGGTATCAGCAAAAACCAGGGAAAGCCCCT |
| | | sequence | AAGCTCCTGATCTATGCTGCATCCACTTTGCAAAGTGGGGTCCC |
| | | | ATCAAGGTTCAGCGGCAGTGGATCTGGGACAGAATTCACTCTCA |
| | | | CAATCAGCAGCCTGCAGCCTGAAGATTTTGCAACTTATTACTGT |
| | | | CAACAGCTTAGTAGTCACCCTATCATCTTCGGCCAAGGGACACG |
| | | | ACTGGAGATTAAA |
| 532 | YANG-1107 | VL domain | DIQLTQSPSFLSASVGDRVTITCWASQGISTYLAWYQQKPGKAP |
| | | amino | KLLIYAASTLQSGVPSRFSGSGSGTEFTLTISSLQPEDFATYYC |
| | | acid | QQLSSHPIIFGQGTRLEIK |
| | | sequence | |
| 176 | YANG-1107 | LCDR1 | QGISTY |
| 18 | YANG-1107 | LCDR2 | AAS |
| 533 | YANG-1107 | LCDR3 | QQLSSHPII |
| 534 | YANG-1108 | VH domain | CAGGTGCAGCTGGTGGAATCTGGGGGAGGCGTGGTCCACCCTGG |
| | | nucleic | GAGGTCCCTGAGACTCTCCTGTGTAGTCTCTGGATTCACCTTCA |
| | | acid | GTGGCTATGGCATGTACTGGGTCCGCCAGGCTCCAGGCAAGGGG |
| | | sequence | CTGGAGTGGGTGGCAGTCATATCAAAGGATGGAAGTGATAAATA |
| | | | CTATGCAGACTCCGTGAAGGGCCGATTCACCATTTCCAGAGACA |
| | | | ATTCCAAGAACACACTGAATCTGCAAATGAACAGCCTGAGAGCT |
| | | | GAGGACACGGCTGTGTTTTACTGTGCGAAAGAAAGGACTTTCCA |
| | | | AGGTTCGGGGAGTTATTATAACAACTACTTTTATTATGGTATGG |
| | | | ACGTCTGGGGCCAAGGGACCACGGTCACCGTCTCCTCA |
| 535 | YANG-1108 | VH domain | QVQLVESGGGVVHPGRSLRLSCVVSGFTFSGYGMYWVRQAPGKG |
| | | amino | LEWVAVISKDGSDKYYADSVKGRFTISRDNSKNTLNLQMNSLRA |
| | | acid | EDTAVFYCAKERTFQGSGSYYNNYFYYGMDVWGQGTTVTVSS |
| | | sequence | |
| 536 | YANG-1108 | HCDR1 | GFTFSGYG |
| 537 | YANG-1108 | HCDR2 | ISKDGSDK |
| 538 | YANG-1108 | HCDR3 | AKERTFQGSGSYYNNYFYYGMDV |
| 539 | YANG-1108 | VL domain | GCCATCCAGTTGACCCAGTCTCCATCCTCCCTGTCTGCATCTGT |
| | | nucleic | AGGAGACAGAGTCACCATCACTTGCCGGGCAAGTCAGGGCATTA |
| | | acid | GCAGTGCTTTAGCCTGGTATCAGCAGAAACCAGGGAAAGCTCCT |
| | | sequence | AACCTCCTGATCTATGATGCCTCCAGTTTGGAAAGTGGGGTCCC |
| | | | ATCAAGGTTCAGCGGCAGTGGATTTGGGACAGATTTCACTCTCA |
| | | | CCATCAACAGCCTGCAGCCTGAAGATTTTGCAAGTTATTATTGT |
| | | | CAACAGTTTAAGAGTTATCCGCTCACTTTCGGCGGAGGGACCAA |
| | | | GGTGGAGATCAAG |
| 540 | YANG-1108 | VL domain | AIQLTQSPSSLSASVGDRVTITCRASQGISSALAWYQQKPGKAP |
| | | amino | NLLIYDASSLESGVPSRFSGSGFGTDFTLTINSLQPEDFASYYC |
| | | acid | QQFKSYPLTFGGGTKVEIK |
| | | sequence | |
| 541 | YANG-1108 | LCDR1 | QGISSA |
| 28 | YANG-1108 | LCDR2 | DAS |
| 542 | YANG-1108 | LCDR3 | QQFKSYPLT |
| 543 | YANG-1109 | VH domain | GAGGTGCAGCTGGTGGAGTCTGGAGGAGGCTTGATCCAGCCTGG |
| | | nucleic | GGGGTCCCTGAGACTCTCCTGTGGAGTCTCAGGACTCACCGTCA |
| | | acid | GTAGAAACTATATGAATTGGGTCCGCCAGGCTCCAGGGAAGGGG |
| | | sequence | CTGGAGTGGGTCTCAGTTATTTATAGCGGTGGTAGCACATTCTA |
| | | | CGCAGACTCCGTGAAGGGCCGATTCACCATCTCCAGAGACAACT |
| | | | CCAAAAACACGCTGTATCTTCAAATGATCAGCCTGAGAGCCGAG |
| | | | GACTCGGGCGTGTATTACTGTGCGCGAGAAGGATTTGGTATGGA |
| | | | CGTCTGGGGCCAAGGGACCACGGTCACCGTCTCCTCA |
| 544 | YANG-1109 | VH domain | EVQLVESGGGLIQPGGSLRLSCGVSGLTVSRNYMNWVRQAPGKG |
| | | amino | LEWVSVIYSGGSTFYADSVKGRFTISRDNSKNTLYLQMISLRAE |
| | | acid | DSGVYYCAREGFGMDVWGQGTTVTVSS |
| | | sequence | |
| 545 | YANG-1109 | HCDR1 | GLTVSRNY |
| 14 | YANG-1109 | HCDR2 | IYSGGST |
| 546 | YANG-1109 | HCDR3 | AREGFGMDV |
| 547 | YANG-1109 | VL domain | GACATCCAGTTGACCCAGTCTCCATCCTTCCTGTCTGCATCTGT |
| | | nucleic | AGGAGACAGAGTCACCATCACTTGCTGGGCCAGTCAGGGCATTA |
| | | acid | GCAATTATTTAGCCTGGTATCAGCAAAAACCAGGGAAAGCCCCT |
| | | sequence | AAGCTCCTGATCTATTCTGCATCCACTTTGCAAAGTGGGGTCCC |
| | | | ATCAAGGTTCAGCGGCAGTGGATCTGGGACAGAATTCACTCTCA |
| | | | CAATCAGCAACCTGCAGCCTGCAGATATTGCAACTTATTACTGT |
| | | | CAACTCCTTAATAGTCATCCGTGCAGTTTTGGCCAGGGGACCAA |
| | | | GCTGGAGATCAAA |
| 548 | YANG-1109 | VL domain | DIQLTQSPSFLSASVGDRVTITCWASQGISNYLAWYQQKPGKAP |
| | | amino | KLLIYSASTLQSGVPSRFSGSGSGTEFTLTISNLQPADIATYYC |
| | | acid | QLLNSHPCSFGQGTKLEIK |
| | | sequence | |
| 549 | YANG-1109 | LCDR1 | QGISNY |
| 517 | YANG-1109 | LCDR2 | SAS |
| 550 | YANG-1109 | LCDR3 | QLLNSHPCS |
| 551 | YANG-1110 | VH domain | CAGGTGCAGCTGGCGGAGTCTGGGGGAGGCGTGGTCCAGCCTGG |
| | | nucleic | GAGGTCCCTGAGACTCTCCTGTGTAGTCTCTGGATTCACCTTCA |
| | | acid | GTAACTATGGCATGCACTGGGTCCGCCAGGCTCCAGGCAAGGGC |
| | | sequence | CTGGAGTGGGTGGTATTTATATCAAATGATGGAAGTAATGAAGA |
| | | | CTTTGTAGACTCCGTGAAGGGCCGATTCACCATCTCCAGAGACA |
| | | | ATTCCAAGAACACGCTGTATCTGCAAATGAACAGCCTGAGAGCT |
| | | | GAGGACACGGCTGTGTATTACTGTGCGAAAGGAGGGTATTTCTA |
| | | | TGGTTCGGGGAATTATTACTACTACTACGGTATGGACGTCTGGG |
| | | | GCCAAGGGACCACGGTCACCGTCTCCTCA |
| 552 | YANG-1110 | VH domain | QVQLAESGGGVVQPGRSLRLSCVVSGFTFSNYGMHWVRQAPGKG |
| | | amino | LEWVVFISNDGSNEDFVDSVKGRFTISRDNSKNTLYLQMNSLRA |
| | | acid | EDTAVYYCAKGGYFYGSGNYYYYYGMDVWGQGTTVTVSS |
| | | sequence | |
| 553 | YANG-1110 | HCDR1 | GFTFSNYG |
| 554 | YANG-1110 | HCDR2 | ISNDGSNE |
| 555 | YANG-1110 | HCDR3 | AKGGYFYGSGNYYYYYGMDV |
| 556 | YANG-1110 | VL domain | CAGCCTGTGCTGACTCAATCATCCTCTGCCTCTGCTTCCCTGGG |
| | | nucleic | ATCCTCGGTCAAGCTCACCTGCACTCTGAGCAGTGTGCACAGTA |
| | | acid | ACCACATCATCGCATGGCATCAGCAGCAGCCAGGGAAGGCCCCT |
| | | sequence | CGGTACTTGATGAAGCTTGAAGGTAGTGGAAGGTACAGTAAGGG |
| | | | GAGCGGAGTTCCTGATCGCTTCTCAGGCTCCAGCTCTGAGGCTG |
| | | | ACCGCTACCTCACCATCTCCAACCTCCAGTCTGAGGATGAGGCT |
| | | | GATTATTACTGTGAGACCTGGGACAATAATACTTATGTCTTCGG |
| | | | ATCTGGGACCAAAGTCACCGTCCTA |
| 557 | YANG-1110 | VL domain | QPVLTQSSSASASLGSSVKLTCTLSSVHSNHIIAWHQQQPGKAP |
| | | amino | RYLMKLEGSGRYSKGSGVPDRFSGSSSEADRYLTISNLQSEDEA |
| | | acid | DYYCETWDNNTYVFGSGTKVTVL |
| | | sequence | |
| 558 | YANG-1110 | LCDR1 | SVHSNHI |
| 559 | YANG-1110 | LCDR2 | LEGSGRY |
| 560 | YANG-1110 | LCDR3 | ETWDNNTYV |
| 561 | YANG-1111 | VH domain | GAGGTGCAGCTGGTGGAGTCTGGGGGAGGCTTGATACAGCCTGG |
| | | nucleic | GGGGTCCCTGAGACTCTCCTGTGCAGCCTCTGGATTCACCTTCA |
| | | acid | GTAGCTATAGCATAAACTGGGTCCGCCAGGCTCCAGGGAAGGGG |
| | | sequence | CTGGAGTGGGTTTCATTCATTAGTGGTAGTAGTACTACCATATA |
| | | | CTACGCAGACTCTGTGAAGGGCCGATTCACCATCTCCAGAGACA |
| | | | GTGCCAAGAACTCACTGTATCTGCAAATGAACAGCCTGAGAGAC |
| | | | GAGGACACGGCTGTGTATTACTGTGCGAGAGGCCTCCGATCGAG |
| | | | TATAGCACCTCGTCCGGACTACTTTGACTCCTGGGGCCAGGGAA |
| | | | TTTTGGTCACCGTCTCCTCA |
| 562 | YANG-1111 | VH domain | EVQLVESGGGLIQPGGSLRLSCAASGFTFSSYSINWVRQAPGKG |
| | | amino | LEWVSFISGSSTTIYYADSVKGRFTISRDSAKNSLYLQMNSLRD |
| | | acid | EDTAVYYCARGLRSSIAPRPDYFDSWGQGILVTVSS |
| | | sequence | |
| 563 | YANG-1111 | HCDR1 | GFTFSSYS |
| 564 | YANG-1111 | HCDR2 | ISGSSTTI |
| 565 | YANG-1111 | HCDR3 | ARGLRSSIAPRPDYFDS |
| 566 | YANG-1111 | VL domain | TCCTATGTACTGACTCAGCCACCCTCAGTGTCAGTGGCCCCAGG |
| | | nucleic | AAAGACGGCCAGGATTACCTGTGGGGGAAACAACATTGGAAGTA |
| | | acid | AAGGTGTACACTGGTACCAGCAGAAGCCAGGCCAGGCCCCTGTA |
| | | sequence | CTGGTCATCTATTATGATAGCGACCGGCCCTCAGGGATCCCTGA |
| | | | GCGATTCTCTGGCTCCAACTCTGGGAACACGGCCACCCTGACCA |
| | | | TCAGCAGGGTCGAAGCCGGGGATGAGGCCGACTATTCCTGTCAG |
| | | | GTGTGGGATAGTAGTAGTGATCATCCGGTATTCGGCGGAGGGAC |
| | | | CAAGCTGACCGTCCAA |
| 567 | YANG-1111 | VL domain | SYVLTQPPSVSVAPGKTARITCGGNNIGSKGVHWYQQKPGQAPV |
| | | amino | LVIYYDSDRPSGIPERFSGSNSGNTATLTISRVEAGDEADYSCQ |
| | | acid | VWDSSSDHPVFGGGTKLTVQ |
| | | sequence | |
| 2364 | YANG-1111 | LCDR1 | NIGSKG |
| 568 | YANG-1111 | LCDR2 | YDS |
| 569 | YANG-1111 | LCDR3 | QVWDSSSDHPV |
| 570 | YANG-1112 | VH domain | CAGATCACCTTGAAGGAGTCTGGTCCTACGCTGGTGAAACCCAC |
| | | nucleic | ACAGACCCTCACGCTGACCTGCACCTTCTCTGGGTTCTCACTCA |
| | | acid | GCACTGGTGGAGTGGCTGTGGGCTGGATCCGTCAGCCCCCAGGA |
| | | sequence | AAGGCCCTGGAGTGGCTTGCAATCATTTATTGGGATGATGATAA |
| | | | GCGTTACAGCCCATCTCTGAAGAGCAGGCTCACCATCACCAAGG |
| | | | ACACCTCCAAAAACCAGGTGGTCCTTACAATGACCAACATGGAC |
| | | | CCTGTGGACACAGCCACATATTACTGTGCACACTGGGGAAAAGA |
| | | | TTCTTTTGATATCTGGGGCCAAGGGACAATGGTCACCGTCTCTT |
| | | | CA |
| 571 | YANG-1112 | VH domain | QITLKESGPTLVKPTQTLTLTCTFSGFSLSTGGVAVGWIRQPPG |
| | | amino | KALEWLAIIYWDDDKRYSPSLKSRLTITKDTSKNQVVLTMTNMD |
| | | acid | PVDTATYYCAHWGKDSFDIWGQGTMVTVSS |
| | | sequence | |
| 572 | YANG-1112 | HCDR1 | GFSLSTGGVA |
| 573 | YANG-1112 | HCDR2 | IYWDDDK |
| 574 | YANG-1112 | HCDR3 | AHWGKDSFDI |
| 575 | YANG-1112 | VL domain | TCCTATGAGCTGACACAGCCACCCTCGGTGTCAGTGTCCCCAGG |
| | | nucleic | ACAAACGGCCAGGATCACCTGCTCTGGAGATGCATTGCCAAGAA |
| | | acid | AATATGCTTATTGGTACCAGCAGAAGTCAGGCCAGGCCCCTGTG |
| | | sequence | CTGGTCATCTATGAGGACAATAACCGACCCTCCGGGATCCCTGA |
| | | | GAGATTCTCTGGCTCCAGCTCAGGGACAACGGCCACCTTGACTG |
| | | | TCAGTGGGGCCCAGGTGGAAGATGAAGCTGACTACTACTGTTAC |
| | | | TCAACAGACACCAGTGGTTATGTGGTATTCGGCGGAGGGACCAA |
| | | | GTTGACCGTCCTA |
| 576 | YANG-1112 | VL domain | SYELTQPPSVSVSPGQTARITCSGDALPRKYAYWYQQKSGQAPV |
| | | amino | LVIYEDNNRPSGIPERFSGSSSGTTATLTVSGAQVEDEADYYCY |
| | | acid | STDTSGYVVFGGGTKLTVL |
| | | sequence | |
| 577 | YANG-1112 | LCDR1 | ALPRKY |
| 578 | YANG-1112 | LCDR2 | EDN |
| 579 | YANG-1112 | LCDR3 | YSTDTSGYVV |
| 580 | YANG-1112a | VH domain | CAGATCACCTTGAAGGAGTCTGGTCCTACGCTGGTGAAACCCAC |
| | | nucleic | ACAGACCCTCACGGTGACCTGCACCTTCTCTGGGTTCTCACTCA |
| | | acid | CCACTGGTGGAGAGGCTGTGGGCTGGATCCGTCAGCCCCCAGGA |
| | | sequence | AAGGCCCTGGAGTGGCTTGCAATCATTTATTGGGATGATGATAA |
| | | | GCGTTACAGCCCATCTCTGAAGAGCAGGCTCACCATCACCAAGG |
| | | | ACACCTCCAGAAACCAGGTGGTCCTTATAATGACCAACATGGAC |
| | | | CCTATGGACACAGCCACATATTACTGTGCACACTGGGGAAAAGA |
| | | | TTCTTTTGATATCTGGGGCCAAGGGACAATGGTCACCGTCTCTT |
| | | | CA |
| 581 | YANG-1112a | VH domain | QITLKESGPTLVKPTQTLTVTCTFSGFSLTTGGEAVGWIRQPPG |
| | | amino | KALEWLAIIYWDDDKRYSPSLKSRLTITKDTSRNQVVLIMTNMD |
| | | acid | PMDTATYYCAHWGKDSFDIWGQGTMVTVSS |
| | | sequence | |
| 582 | YANG-1112a | HCDR1 | GFSLTTGGEA |
| 583 | YANG-1112a | HCDR2 | IYWDDDK |
| 584 | YANG-1112a | HCDR3 | AHWGKDSFDI |
| 585 | YANG-1112a | VL domain | TCCCATGAGCTGACACAGCCACCCTCGGTGTCAGTGTCCCCAGG |
| | | nucleic | ACAAACGGCCAGGATCACCTGCTCTGGAGATGAATTGTCAAGAA |
| | | acid | AATATGCTTATTGGTACCAGCAGAAGTCAGGCCAGGCCCCTGTG |
| | | sequence | CTGGTCATCTATGAGGACAACAAACGACCCTCCGGGATCCCTGA |
| | | | GAGATTCTCTGGCTCCAGCTCAGGGACAACGGCCACCTTGACTG |
| | | | TCAGTGGGGCCCAGGTGGACGATGAAGCTGACTACTACTGTTAC |
| | | | TCAACAGACACCAGTGGTTATGTGGTATTCGGCGGAGGGACCAA |
| | | | GTTGACCGTCCTA |
| 586 | YANG-1112a | VL domain | SHELTQPPSVSVSPGQTARITCSGDELSRKYAYWYQQKSGQAPV |
| | | amino | LVIYEDNKRPSGIPERFSGSSSGTTATLTVSGAQVDDEADYYCY |
| | | acid | STDTSGYVVFGGGTKLTVL |
| | | sequence | |
| 587 | YANG-1112a | LCDR1 | ELSRKY |
| 578 | YANG-1112a | LCDR2 | EDN |
| 588 | YANG-1112a | LCDR3 | YSTDTSGYVV |
| 589 | YANG-1112b | VH domain | CAGATCACCTTGAAGGAGTCTGGTCCTACGCTGGTGAAACCCAC |
| | | nucleic | ACAGACCCTCACGCTGACCTGCACCTTCTCTGGGTTCTCACTCA |
| | | acid | CCACTGCTGGAGCGGCTGTGGGCTGGATCCGTCAGCCCCCAGGA |
| | | sequence | AAGGCCCTGGAATGGCTTGCAATCATTTACTGGGATGATGATAA |
| | | | GCGTTACAGTCCATCTCTGAAGAACAGGCTCACCATCACCAAGG |
| | | | ACACCTCCAAAAACCAGGTTGTCCTTACAATGACCAACATGGAC |
| | | | CCTGTGGACACAGCCACATATTACTGTGCACACTGGGGAAAAGA |
| | | | TTCTTTTGATATCTGGGGCCAAGGGACAATGGTCACCGTCTCTT |
| | | | CA |
| 590 | YANG-1112b | VH domain | QITLKESGPTLVKPTQTLTLTCTFSGESLTTAGAAVGWIRQPPG |
| | | amino | KALEWLAIIYWDDDKRYSPSLKNRLTITKDTSKNQVVLTMTNMD |
| | | acid | PVDTATYYCAHWGKDSFDIWGQGTMVTVSS |
| | | sequence | |
| 591 | YANG-1112b | HCDR1 | GFSLTTAGAA |
| 592 | YANG-1112b | HCDR2 | IYWDDDK |
| 593 | YANG-1112b | HCDR3 | AHWGKDSFDI |
| 594 | YANG-1112b | VL domain | TCCTATGAGCTGACACAGCCACCCTCGGTGTCAGTGCCCCCAGG |
| | | nucleic | ACAAACGGCCAGGATCACCTGCTCTGGAGATGCATTGCCAACAA |
| | | acid | AATATGCTTATTGGTACCAGCAGAAGTCAGGCCAGGCCCCTGTG |
| | | sequence | CTGGTCATCTATGAGGACAACAAACGACCCTCCGGGATCCCTGA |
| | | | GAGATTCTCTGGCTCCAGCTCAGGGACAACGGCCACCTTGACTG |
| | | | TCAGTGGGGCCCAGGTGGAGGATGAAGCTGACTTCTACTGTTAT |
| | | | TCAACAGACACCAGTGGTTATGTAGTATTCGGCGGAGGGACCAA |
| | | | GTTGACCGTCCTA |
| 595 | YANG-1112b | VL domain | SYELTQPPSVSVPPGQTARITCSGDALPTKYAYWYQQKSGQAPV |
| | | amino | LVIYEDNKRPSGIPERFSGSSSGTTATLTVSGAQVEDEADFYCY |
| | | acid | STDTSGYVVFGGGTKLTVL |
| | | sequence | |
| 596 | YANG-1112b | LCDR1 | ALPTKY |
| 578 | YANG-1112b | LCDR2 | EDN |
| 597 | YANG-1112b | LCDR3 | YSTDTSGYVV |
| 598 | YANG-1112c | VH domain | CAGATCACCTTGAAGGAGTCTGGTCCTACGCTGGTGAAACCCAC |
| | | nucleic | ACAGACCCTCACGGTGACCTGCACCTTCTCTGGGTTCTCACTCA |
| | | acid | GCACTGGTGGAGAGGCTGTGGGCTGGATCCGTCAGCCCCCAGGA |
| | | sequence | AAGGCCCTGGAGTGGCTTGCAATCATTTATTGGGATGATGATAA |
| | | | GCGTTACAGCCCATCTCTGAAGAGTAGGCTCACCATCACCAAGG |
| | | | ACACCTCCAAAAACCAGGTGGTCCTTACAATGACCAACATGGAC |
| | | | CCTATGGACACAGCCACATATTACTGTGCACACTGGGGAAAAGA |
| | | | TTCTTTTGATATCTGGGGCCAAGGGACAATGGTCACCGTCTCTT |
| | | | CA |
| 599 | YANG-1112c | VH domain | QITLKESGPTLVKPTQTLTVTCTFSGFSLSTGGEAVGWIRQPPG |
| | | amino | KALEWLAIIYWDDDKRYSPSLKSRLTITKDTSKNQVVLTMTNMD |
| | | acid | PMDTATYYCAHWGKDSFDIWGQGTMVTVSS |
| | | sequence | |
| 600 | YANG-1112c | HCDR1 | GFSLSTGGEA |
| 601 | YANG-1112c | HCDR2 | IYWDDDK |
| 602 | YANG-1112c | HCDR3 | AHWGKDSFDI |
| 603 | YANG-1112c | VL domain | TCCCATGAGCTGACACAGCCACCCTCGGTGTCAGTGTCCCCAGG |
| | | nucleic | ACAAACGGCCAGGATCACCTGCTCTGGAGATGAATTGTCAAGAA |
| | | acid | AATATGCTTATTGGTACCAGCAGAAGTCAGGCCAGGCCCCTGTG |
| | | sequence | CTGGTCATCTATGAGGACAACAAACGACCCTCCGGGATCCCTGA |
| | | | GAGATTCTCTGGCTCCAGCTCAGGGACAACGGCCACCTTGACTG |
| | | | TCAGTGGGGCCCAGGTGGACGATGAAGCTGACTACTACTGTTAC |
| | | | TCAACAGACACCAGTGGTTATGTGGTATTCGGCGGAGGGACCAA |
| | | | GTTGACCGTCCTA |
| 504 | YANG-1112c | VL domain | SHELTQPPSVSVSPGQTARITCSGDELSRKYAYWYQQKSGQAPV |
| | | amino | LVIYEDNKRPSGIPERFSGSSSGTTATLTVSGAQVDDEADYYCY |
| | | acid | STDTSGYVVFGGGTKLTVL |
| | | sequence | |
| 605 | YANG-1112c | LCDR1 | ELSRKY |
| 578 | YANG-1112c | LCDR2 | EDN |
| 606 | YANG-1112c | LCDR3 | YSTDTSGYVV |
| 607 | YANG-1113 | VH domain | GAGGTGCAGCTGGTGGAGTCTGGGGGAGGCCTGGTCAAGCCTGG |
| | | nucleic | GGGGTCCCTGAGACTCTCCTGTGCAGCCTCTGGATTCACCTTCA |
| | | acid | GTAGCTATAGCATGAACTGGGTCCGCCAGGCTCCAGGGAAGGGG |
| | | sequence | CTGGAGTGGGTCTCATCCATTAGTACTGGTAGTAGTTACATATT |
| | | | CTACGCAGACTCAGTGAAGGGCCGATTCACCATGTCCAGAGACA |
| | | | ACGCCAAGAACTCACTGTATCTACAAATGAACAGCCTGAGAGCC |
| | | | GAAGACACAGCTGTGTATTACTGTGCGAAAACTGGGGATCTTCC |
| | | | TTTCTTTGACTACTGGGGCCAGGGAACCCCGGTCACCGTCTCCT |
| | | | CA |
| 608 | YANG-1113 | VH domain | EVQLVESGGGLVKPGGSLRLSCAASGFTFSSYSMNWVRQAPGKG |
| | | amino | LEWVSSISTGSSYIFYADSVKGRFTMSRDNAKNSLYLQMNSLRA |
| | | acid | EDTAVYYCAKTGDLPFFDYWGQGTPVTVSS |
| | | sequence | |
| 609 | YANG-1113 | HCDR1 | GFTFSSYS |
| 610 | YANG-1113 | HCDR2 | ISTGSSYI |
| 611 | YANG-1113 | HCDR3 | AKTGDLPFFDY |
| 612 | YANG-1113 | VL domain | CAGTCTGCCCTGACTCAGCCTGCCTCCGTGTCTGGGTCTCCTGG |
| | | nucleic | ACAGTCGATCACCATCTCCTGCACTGGAACCAGCAGTGACGTTG |
| | | acid | GTCGTTATAACTATGTCTCCTGGTACCAACAGCACCCAGGCAAA |
| | | sequence | GCCCCCAAACTCATGATTTATGAGGTCAGTAATCGGCCCTCAGG |
| | | | GGTTTCTAATCGCTTCTCTGGCTCCAAGTCTGGCAACACGGCCT |
| | | | CCCTGACCATCTCTGGGCTCCAGGCTGAGGACGAGGCTGATTAT |
| | | | TACTGCAGCTCATATACAAGCAGCAGCACTATGGTATTCGGCGG |
| | | | AGGGACCAAGTTGACTGTCTTA |
| 613 | YANG-1113 | VL domain | QSALTQPASVSGSPGQSITISCTGTSSDVGRYNYVSWYQQHPGK |
| | | amino | APKLMIYEVSNRPSGVSNRFSGSKSGNTASLTISGLQAEDEADY |
| | | acid | YCSSYTSSSTMVFGGGTKLTVL |
| | | sequence | |
| 614 | YANG-1113 | LCDR1 | SSDVGRYNY |
| 615 | YANG-1113 | LCDR2 | EVS |
| 616 | YANG-1113 | LCDR3 | SSYTSSSTMV |
| 617 | YANG-1114 | VH domain | GAGGTGCACCTGGTGGAGTCTGTGGGAGGCCTGGTCAAGCCGGG |
| | | nucleic | GGGGTCCCTGAGACTCTCCTGTGCAGCCTCTGGATTCACCTTCA |
| | | acid | GTAGCTATAGCATGAACTGGGTCCGCCAGGCTCCAGGGAAGGGG |
| | | sequence | CTGGAGTGGGTCTCATCCATTAGTACTGGGAGTAGTTACATATT |
| | | | CTACGCAGACTCAGTGAAGGGCCGATTCACCATCTCCAGAGACA |
| | | | ACGCCAAGAACTCACTGTATCTGCAAATGAACAGCCTGAGAGCC |
| | | | GAGGACACAGCTGTGTATTACTGTGCGAAAACTGGGGATCTTCC |
| | | | TTTCTTTGACTACTGGGGCCAGGGAACCCTGGTCACCGTCTCCT |
| | | | CA |
| 618 | YANG-1114 | VH domain | EVHLVESVGGLVKPGGSLRLSCAASGFTFSSYSMNWVRQAPGKG |
| | | amino | LEWVSSISTGSSYIFYADSVKGRFTISRDNAKNSLYLQMNSLRA |
| | | acid | EDTAVYYCAKTGDLPFFDYWGQGTLVTVSS |
| | | sequence | |
| 619 | YANG-1114 | HCDR1 | GFTFSSYS |
| 620 | YANG-1114 | HCDR2 | ISTGSSYI |
| 621 | YANG-1114 | HCDR3 | AKTGDLPFFDY |
| 622 | YANG-1114 | VL domain | CAGTCTGCCCTGACTCAGCCTGCCTCCGTGTCTGGGTCTCCTGG |
| | | nucleic | ACAGTCGATCACCATCTCCTGCACTGGAACCAGCAGTGACGTTG |
| | | acid | GTCGTTATAACTATGTCTCCTGGTACCAACAGCACCCAGGCAAA |
| | | sequence | GCCCCCAAACTCATGATTTATGAGGTCAGTAATCGGCCCTCAGG |
| | | | GGTTTCTAATCGCTTCTCTGGCTCCAAGTCTGGCAACACGGCCT |
| | | | CCCTGACCATCTCTGGGCTCCAGGCTGAGGACGAGGCTGATTAT |
| | | | TACTGCAGCTCATATACAAGCAGCAGCACTATGGTATTCGGCGG |
| | | | AGGGACCAAGCTGACCGTCCTA |
| 623 | YANG-1114 | VL domain | QSALTQPASVSGSPGQSITISCTGTSSDVGRYNYVSWYQQHPGK |
| | | amino | APKLMIYEVSNRPSGVSNRFSGSKSGNTASLTISGLQAEDEADY |
| | | acid | YCSSYTSSSTMVFGGGTKLTVL |
| | | sequence | |
| 624 | YANG-1114 | LCDR1 | SSDVGRYNY |
| 615 | YANG-1114 | LCDR2 | EVS |
| 616 | YANG-1114 | LCDR3 | SSYTSSSTMV |
| 2365 | YANG-1115 | VH domain | GAGGTGCAGCTGGTGGAGTCTGGGGGAGGCCTGGTCAAGCCTGG |
| | | nucleic | GGGGTCCCTGAGACTCTCCTGTGCAGCCTCTGGATTCACCTTCA |
| | | acid | GTAGCTATAGCATGAACTGGGTCCGCCAGGCTCCAGGGAAGGGG |
| | | sequence | CTGGAGTGGGTCTCGTCCATTAGTACTGGTAGTAGTTACATATT |
| | | | CTACGCAGACTCAGTGAAGGGCCGATTCACCATCTCCAGAGACA |
| | | | ACGCCAAGAACTCACTGTATCTGCAAATGAACAGCCTGAGAGCC |
| | | | GAGGACACAGCTGTATATTACTGTGCGAAAACTGGGGATCTTCC |
| | | | TTTCTTTGACTACTGGGGCCAGGGAACCCTGGTCACCGTCTCCT |
| | | | CA |
| 2366 | YANG-1115 | VH domain | EVQLVESGGGLVKPGGSLRLSCAASGFTFSSYSMNWVRQAPGKG |
| | | amino | LEWVSSISTGSSYIFYADSVKGRFTISRDNAKNSLYLQMNSLRA |
| | | acid | EDTAVYYCAKTGDLPFFDYWGQGTLVTVSS |
| | | sequence | |
| 619 | YANG-1115 | HCDR1 | GFTFSSYS |
| 620 | YANG-1115 | HCDR2 | ISTGSSYI |
| 621 | YANG-1115 | HCDR3 | AKTGDLPFFDY |
| 2367 | YANG-1115 | VL domain | CAGTCTGCCCTGACTCAGCCTGCCTCCGTGTCTGGGTCTCCTGG |
| | | nucleic | ACAGTCGATCACCATCTCCTGCACTGGAACCAGCAGTGACGTTG |
| | | acid | GTCGTTATAACTATGTCTCCTGGTACCAACAGCACCCAGGCAAA |
| | | sequence | GCCCCCAAACTCATGATTTATGAGGTCAGTAATCGGCCCTCAGG |
| | | | GGTTTCTAATCGCTTCTCTGGCTCCAAGTCTGGCAACACGGCCT |
| | | | CCCTGACCATCTCTGGGCTCCAGGCTGAGGACGAGGCTGATTAT |
| | | | TACTGCAGCTCATATACAAGCAGCAGCACTATGGTATTCGGCGG |
| | | | AGGGACCAAGCTGTCCGTCCTA |
| 2368 | YANG-1115 | VL domain | QSALTQPASVSGSPGQSITISCTGTSSDVGRYNYVSWYQQHPGK |
| | | amino | APKLMIYEVSNRPSGVSNRFSGSKSGNTASLTISGLQAEDEADY |
| | | acid | YCSSYTSSSTMVFGGGTKLSVL |
| | | sequence | |
| 624 | YANG-1115 | LCDR1 | SSDVGRYNY |
| 615 | YANG-1115 | LCDR2 | EVS |
| 625 | YANG-1115 | LCDR3 | SSYTSSSTMV |
| 626 | YANG-1116 | VH domain | GAGGTACAGCTGGTGGAGTCTGGGGGAGGCCTGGTCAAGCCTGG |
| | | nucleic | GGGGTCCCTGAGACTCTCCTGTGAAGCCTCTGGATTCAATTTCA |
| | | acid | GAAGCTATGCCCTGAACTGGGTCCGCCAGGCTCCAGGGAAGGGG |
| | | sequence | CTGGAGTGGGTCTCATCCATTAGTACTGGTAGTAGTTACATATT |
| | | | CTACGCAGACTCAGTGAAGGGCCGATTCACCATCTCCAGAGACA |
| | | | ACGCCAAGAACTCACTGTCTCTGCAAATGAACAGCCTGAGAGCC |
| | | | GAGGACACAGCTGTGTATTACTGTGCGAAAACTGGGGATCTTCC |
| | | | TTTCTTTGACTACTGGGGCCAGGGAACCCTGGTCACCGTCTCCT |
| | | | CA |
| 627 | YANG-1116 | VH domain | EVQLVESGGGLVKPGGSLRLSCEASGENFRSYALNWVRQAPGKG |
| | | amino | LEWVSSISTGSSYIFYADSVKGRFTISRDNAKNSLSLQMNSLRA |
| | | acid | EDTAVYYCAKTGDLPFFDYWGQGTLVTVSS |
| | | sequence | |
| 628 | YANG-1116 | HCDR1 | GENFRSYA |
| 629 | YANG-1116 | HCDR2 | ISTGSSYI |
| 630 | YANG-1116 | HCDR3 | AKTGDLPFFDY |
| 631 | YANG-1116 | VL domain | CAGTCTGCCCTGACTCAGCCTGCCTCCGTGTCTGGGTCTCCTGG |
| | | nucleic | ACAGTCGATCACCATCTCCTGCACTGGAACCAGCAGTGACGTTG |
| | | acid | GTCGTTATAACTATGTCTCCTGGTACCAACAGCACCCAGGCAAA |
| | | sequence | GCCCCCAAACTCATGATTTATGAGGTCAGTAATCGGCCCTCAGG |
| | | | GGTTTCTAATCGCTTCTCTGGCTCCAAGTCTGGCAACACGGCCT |
| | | | CCCTGACCATCTCTGGGCTCCAGGCTGAGGACGAGGCTGATTAT |
| | | | TACTGCATCTCATATACAAGCAGCAGCACTATGGTATTCGGCGG |
| | | | AGGGACCAAGCTGACCGTCCTA |
| 632 | YANG-1116 | VL domain | QSALTQPASVSGSPGQSITISCTGTSSDVGRYNYVSWYQQHPGK |
| | | amino | APKLMIYEVSNRPSGVSNRFSGSKSGNTASLTISGLQAEDEADY |
| | | acid | YCISYTSSSTMVFGGGTKLTVL |
| | | sequence | |
| 633 | YANG-1116 | LCDR1 | SSDVGRYNY |
| 615 | YANG-1116 | LCDR2 | EVS |
| 634 | YANG-1116 | LCDR3 | ISYTSSSTMV |
| 635 | YANG-1117 | VH domain | GAGGTGCAGCTGGTGGAGTCTGGGGGAGGCCTGGTCAAGCCTGG |
| | | nucleic | GGGGTCCCTGAGACTCTCCTGTGCAGCCTCTGGATTCACCTTCA |
| | | acid | GTAGCTATAGCGTGAACTGGGTCCGCCAGGCTCCAGGGAAGGGG |
| | | sequence | CTGGAGTGGGTCTCATCCATTAGTACTGGTAGTAGTTACATTTT |
| | | | CTACGCAGACTCAGTGAAGGGCCGATTCACCATCTCCAGAGACA |
| | | | ACGCCAAGAACTCACTGTATCTGCAAATGAACAGCCTGAGAGCC |
| | | | GAGGACACAGCTGTGTATTACTGTGTGAAAACTGGGGATCTTCC |
| | | | TTTCTTTGACTACTGGGGCCAGGGAACCCTGGTCACCGTCTCCT |
| | | | CA |
| 636 | YANG-1117 | VH domain | EVQLVESGGGLVKPGGSLRLSCAASGFTFSSYSVNWVRQAPGKG |
| | | amino | LEWVSSISTGSSYIFYADSVKGRFTISRDNAKNSLYLQMNSLRA |
| | | acid | EDTAVYYCVKTGDLPFFDYWGQGTLVTVSS |
| | | sequence | |
| 637 | YANG-1117 | HCDR1 | GFTFSSYS |
| 638 | YANG-1117 | HCDR2 | ISTGSSYI |
| 639 | YANG-1117 | HCDR3 | VKTGDLPFFDY |
| 640 | YANG-1117 | VL domain | CAGTCTGCCCTGACTCAGCCTGCCTCCGTGTCTGGGTCTCCTGG |
| | | nucleic | ACAGTCGATCACCATCTCCTGCACTGGAACCAGAAGTGACGTTG |
| | | acid | GTCGTTATAACTATGTCTCCTGGTACCAACAGCACCCAGGCAAA |
| | | sequence | GCCCCCAAACTCATGATTTATGAGGTCACTAATCGGCCCTCAGG |
| | | | GGTTTCTAATCGCTTCTCTGGCTCCAAGTCTGGCAACACGGCCT |
| | | | CCCTGACCATCTCTGGGCTCCAGGCTGAGGACGAGGCTGATTAT |
| | | | TACTGCAGCTCATATACAAACAACAGCACTATGGTATTCGGCGG |
| | | | AGGGACCAAGCTGACCGTCCTA |
| 641 | YANG-1117 | VL domain | QSALTQPASVSGSPGQSITISCTGTRSDVGRYNYVSWYQQHPGK |
| | | amino | APKLMIYEVTNRPSGVSNRFSGSKSGNTASLTISGLQAEDEADY |
| | | acid | YCSSYTNNSTMVFGGGTKLTVL |
| | | sequence | |
| 642 | YANG-1117 | LCDR1 | RSDVGRYNY |
| 643 | YANG-1117 | LCDR2 | EVT |
| 644 | YANG-1117 | LCDR3 | SSYTNNSTMV |
| 645 | YANG-1118 | VH domain | GAGGTACAGCTGGTGGAGTCTGGGGGAGGCCTGGTCAAGCCTGG |
| | | nucleic | GGGGTCCCTGAGACTCTCCTGTGCAGCCTCTGGATTCACCTTCA |
| | | acid | GTAGCTATGCCCTGAACTGGGTCCGCCAGGCTCCAGGGAAGGGG |
| | | sequence | CTGGAGTGGGTCTCATCCATTAGTACTGGTAGTAGTTACATATT |
| | | | CTACGCAGACTCAGTGAAGGGCCGATTCACCATCTCCAGAGACA |
| | | | ACGCCAAGCACTCACTGTCTCTGCAAATGAACAGCCTGAGAGCC |
| | | | GAGGACACAGCTGTGTATTACTGTGCGAAAACTGGGGATCTTCC |
| | | | TTTCTTTGACTACTGGGGCCAGGGAACCCTGGTCACCGTCTCCT |
| | | | CA |
| 646 | YANG-1118 | VH domain | EVQLVESGGGLVKPGGSLRLSCAASGFTFSSYALNWVRQAPGKG |
| | | amino | LEWVSSISTGSSYIFYADSVKGRFTISRDNAKHSLSLQMNSLRA |
| | | acid | EDTAVYYCAKTGDLPFFDYWGQGTLVTVSS |
| | | sequence | |
| 647 | YANG-1118 | HCDR1 | GFTFSSYA |
| 648 | YANG-1118 | HCDR2 | ISTGSSYI |
| 649 | YANG-1118 | HCDR3 | AKTGDLPFFDY |
| 650 | YANG-1118 | VL domain | CAGTCTGCCCTGACTCAGCCTGCCTCCGTGTCTGGGTCTCCTGG |
| | | nucleic | ACAGTCGATCACCATCTCCTGCACTGGAACCAGCAGTGACGTTG |
| | | acid | GTCGTTATAACTATGTCTCCTGGTACCAACAGCACCCAGGCAAA |
| | | sequence | GCCCCCAAACTCATGATTTATGAGGTCAGTAATCGGCCCTCAGG |
| | | | GGTTTCTAATCGCTTCTCTGGCTCCAAGTCTGGCAACACGGCCT |
| | | | CCCTGACCATCTCTGGGCTCCAGGCTGAGGACGAGGCTGATTAT |
| | | | TACTGCATCTCATATACAAGCAGCAGCACTATGGTGTTCGGCGG |
| | | | AGGGACCAAGTTGACCGTCCTA |
| 651 | YANG-1118 | VL domain | QSALTQPASVSGSPGQSITISCTGTSSDVGRYNYVSWYQQHPGK |
| | | amino | APKLMIYEVSNRPSGVSNRFSGSKSGNTASLTISGLQAEDEADY |
| | | acid | YCISYTSSSTMVFGGGTKLTVL |
| | | sequence | |
| 652 | YANG-1118 | LCDR1 | SSDVGRYNY |
| 615 | YANG-1118 | LCDR2 | EVS |
| 653 | YANG-1118 | LCDR3 | ISYTSSSTMV |
| 654 | YANG-1119 | VH domain | GAGGTGCAGCTGGTGGAGTCTGGGGGAGGCCTGGTCAAGCCTGG |
| | | nucleic | GGGGTCCCTGAGACTCTCCTGTGCAGCCTCTGGATTCACCTTCA |
| | | acid | GTAGCTATAGCATGAACTGGGTCCGCCAGGCTCCAGGGAAGGGG |
| | | sequence | CTGGAGTGGGTCTCATCCATTAGTACTGGTAGTAGTTACATATT |
| | | | CTACGCAGACTCAGTGAAGGGCCGATTCACCATCTCCAGAGACA |
| | | | ACGCCAAGAACTCACTGTATCTGCAAATGAACAGCCTGAGAGCC |
| | | | GAGGACACAGCTGTGTATTACTGTGCGAAAACTGGGGATCTTCC |
| | | | TTTCTTTGACTACTGGGGCCAGGGAACCCTGGTCACCGTCTCCT |
| | | | CA |
| 655 | YANG-1119 | VH domain | EVQLVESGGGLVKPGGSLRLSCAASGFTFSSYSMNWVRQAPGKG |
| | | amino | LEWVSSISTGSSYIFYADSVKGRFTISRDNAKNSLYLQMNSLRA |
| | | acid | EDTAVYYCAKTGDLPFFDYWGQGTLVTVSS |
| | | sequence | |
| 656 | YANG-1119 | HCDR1 | GFTFSSYS |
| 657 | YANG-1119 | HCDR2 | ISTGSSYI |
| 658 | YANG-1119 | HCDR3 | AKTGDLPFFDY |
| 659 | YANG-1119 | VL domain | CAGTCTGCCCTGACTCAGCCTGCCTCCGTGTCTGGGTCTCCTGG |
| | | nucleic | ACAGTCGATCACCATCTCCTGCACTGGAACCAGCAGTGACGTTG |
| | | acid | GTCGTTATAACTATGTCTCCTGGTACCAACAGCACCCAGGCAAA |
| | | sequence | GCCCCCAAACTCATGATTTATGAGGTCAGTAATCGGCCCTCAGG |
| | | | GGTTTCTAATCGCTTCTCTGGCTCCAAGTCTGGCAACACGGCCT |
| | | | CCCTGACCATCTCTGGGCTCCAGGCTGAGGACGAGGCTGATTAT |
| | | | TACTGCAGCTCATATACAAGCAGTAGCACGATGGTATTCGGCGG |
| | | | AGGGACCAAGCTGACCGTCCTA |
| 660 | YANG-1119 | VL domain | QSALTQPASVSGSPGQSITISCTGTSSDVGRYNYVSWYQQHPGK |
| | | amino | APKLMIYEVSNRPSGVSNRFSGSKSGNTASLTISGLQAEDEADY |
| | | acid | YCSSYTSSSTMVFGGGTKLTVL |
| | | sequence | |
| 661 | YANG-1119 | LCDR1 | SSDVGRYNY |
| 615 | YANG-1119 | LCDR2 | EVS |
| 662 | YANG-1119 | LCDR3 | SSYTSSSTMV |
| 663 | YANG-1201 | VH domain | CAGGTGCAGCTGCAGGAGTCGGGCCCAGGACTGGTGAAGCCTTC |
| | | nucleic | GGGGACCCTGTCCCTCACCTGCGCTGTCTCTGGTGACTCCATCA |
| | | acid | GTAATAGTAAATGGTGGAGTTGGGTCCGCCAGCCCCCAGGGAAG |
| | | sequence | GGGCTGGAGTGGATTGGGGAAATCTTTCATAGTGGGAGCACCAA |
| | | | CTACAACCCGTCCCTCAAGAGTCGAGTCACCATTTCAGTAGACA |
| | | | AGTCCCAGAACCAGTTCTCCCTGAAGCTGAACTCTGTGACCGCC |
| | | | GCGGACACGGCCGTGTATTACTGTGCGAGATCAGCATCTCTTTA |
| | | | TTACTACTACGGTGTGGACGTCTGGGGCCAAGGGACCACGGTCA |
| | | | CCGTCTCCTCA |
| 664 | YANG-1201 | VH domain | QVQLQESGPGLVKPSGTLSLTCAVSGDSISNSKWWSWVRQPPGK |
| | | amino | GLEWIGEIFHSGSTNYNPSLKSRVTISVDKSQNQFSLKLNSVTA |
| | | acid | ADTAVYYCARSASLYYYYGVDVWGQGTTVTVSS |
| | | sequence | |
| 665 | YANG-1201 | HCDR1 | GDSISNSKW |
| 666 | YANG-1201 | HCDR2 | IFHSGST |
| 667 | YANG-1201 | HCDR3 | ARSASLYYYYGVDV |
| 668 | YANG-1201 | VL domain | GAAATTGTCTTGACGCAGTCTCCAGGCACCCTGTCTTTGTCTCC |
| | | nucleic | AGGGGAGAGAGCCACCCTCTCCTGCAGGGCCAGTCAGAGTGTTA |
| | | acid | GCAACAACTACTTAGCTTGGTACCAGCAGAAACCTGGCCAGGCT |
| | | sequence | CCCAGGCTCCTCATCTATGGTACATCCAGGAGGGACACTGGCAT |
| | | | CCCAGACAGGTTCAGTGGCAGTGGGTCTGGGACAGACTTCACTC |
| | | | TCACCATCAGCAGACTGGAGCCTGAGGATTTTGCAGTGTATTAC |
| | | | TGTGAGCAGTATGGTAGTTTGCCTCGGACGTTCGGCCAAGGGAC |
| | | | CAAGGTGGAAATCAAA |
| 669 | YANG-1201 | VL domain | EIVLTQSPGTLSLSPGERATLSCRASQSVSNNYLAWYQQKPGQA |
| | | amino | PRLLIYGTSRRDTGIPDRESGSGSGTDFTLTISRLEPEDFAVYY |
| | | acid | CEQYGSLPRTFGQGTKVEIK |
| | | sequence | |
| 670 | YANG-1201 | LCDR1 | QSVSNNY |
| 671 | YANG-1201 | LCDR2 | GTS |
| 672 | YANG-1201 | LCDR3 | EQYGSLPRT |
| 673 | YANG-1202 | VH domain | GAGATGCAGCTGGTGGAGTCTGGGGGAGGCTTGGTCCAGCCTGG |
| | | nucleic | GGGGTCCCTGAGACTCTCCTGTGCAGCCTCTGGATTCACCTTTA |
| | | acid | GTAGTTTTTGGATGCGCTGGGTCCGTCAGGCTCCAGGGAAGGGG |
| | | sequence | CTGGAGTGGGTGGCCAATATAAAGCAAGATGGAACTGAGAAATA |
| | | | CTATGTGGACTCTGTGAAGGGCCGATTCACCATCTCCAGAGACA |
| | | | ACGCCAAGAACTCACTGTATCTGCAAATGAACAGTCTGAGAGCC |
| | | | GAGGACACGGCTGTATATTACTGTGTGAGAGATCGGGTCGGGGG |
| | | | GGACTATTACTACTACGATATGGACGTCTGGGGCCAAGGGACCA |
| | | | CGGTCACCGTCTCCTCA |
| 674 | YANG-1202 | VH domain | EMQLVESGGGLVQPGGSLRLSCAASGFTFSSFWMRWVRQAPGKG |
| | | amino | LEWVANIKQDGTEKYYVDSVKGRFTISRDNAKNSLYLQMNSLRA |
| | | acid | EDTAVYYCVRDRVGGDYYYYDMDVWGQGTTVTVSS |
| | | sequence | |
| 675 | YANG-1202 | HCDR1 | GFTFSSFW |
| 676 | YANG-1202 | HCDR2 | IKQDGTEK |
| 677 | YANG-1202 | HCDR3 | VRDRVGGDYYYYDMDV |
| 678 | YANG-1202 | VL domain | GACATCCAGATGACCCAGTCTCCATCCTCACTGTCTGCATCTGT |
| | | nucleic | AGGAGACAGAGTCACCATCACTTGTCGGGCGAGTCAGGACATTA |
| | | acid | GCAATTATTTAGCCTGGTTTCAGCAGAAACCAGGGAAAGCCCCT |
| | | sequence | AAGTCCCTGATCTATGCTGCATCCAGTTTGCAAAGCGGGGTCCC |
| | | | ATCAAAGTTCAGCGGCAGTGGATCTGGGACAGATTTCACTCTCA |
| | | | CCATCAGCGGCCTGCAGCCTGAAGATTCTGCAACTTATTACTGT |
| | | | CAACAGTATAATAGTTACCCTCGGACGTTCGGCCAAGGGACCAA |
| | | | GGTGGAAATCAAA |
| 679 | YANG-1202 | VL domain | DIQMTQSPSSLSASVGDRVTITCRASQDISNYLAWFQQKPGKAP |
| | | amino | KSLIYAASSLQSGVPSKFSGSGSGTDFTLTISGLQPEDSATYYC |
| | | acid | QQYNSYPRTFGQGTKVEIK |
| | | sequence | |
| 680 | YANG-1202 | LCDR1 | QDISNY |
| 18 | YANG-1202 | LCDR2 | AAS |
| 681 | YANG-1202 | LCDR3 | QQYNSYPRT |
| 682 | YANG-1203 | VH domain | GAAGTGCGGCTGGTGGAGTCTGGGGGAGCCTTGGTACAGCCTGG |
| | | nucleic | CAGGTCCCTGAGACTCTCCTGTGCAGCCTCTGGATTCATCTTTG |
| | | acid | ATGGTTATGCCATGCACTGGGTCCGGCAAGTTCCAGGGAAGGGC |
| | | sequence | CTGGAGTGGGTCTCAGGTATTAGTTGGAATAGTGGTAACATAGG |
| | | | CTATGCGGACTCTGTGAAGGGCCGATTCACCATCTCCAGAGACA |
| | | | ACGCCAAGAACTCCCTGTATCTGCAAATGAACAGTCTGAGAGCT |
| | | | GAGGACACGGCCTTGTATTACTGTGCAAAAGATATAAGGCCTTC |
| | | | GGGAAAGTACTATTACGGTATGGACGTCTGGGGCCAAGGGACCA |
| | | | CGGTCACCGTCTCCTCA |
| 683 | YANG-1203 | VH domain | EVRLVESGGALVQPGRSLRLSCAASGFIFDGYAMHWVRQVPGKG |
| | | amino | LEWVSGISWNSGNIGYADSVKGRFTISRDNAKNSLYLQMNSLRA |
| | | acid | EDTALYYCAKDIRPSGKYYYGMDVWGQGTTVTVSS |
| | | sequence | |
| 684 | YANG-1203 | HCDR1 | GFIFDGYA |
| 685 | YANG-1203 | HCDR2 | ISWNSGNI |
| 686 | YANG-1203 | HCDR3 | AKDIRPSGKYYYGMDV |
| 687 | YANG-1203 | VL domain | GACATCCAGATGACCCAGTCTCCATCCTCCCTGTCTGCATCTGT |
| | | nucleic | AGGAGACAGAGTCACCATCACTTGCCAGGCAAGTCAGGACATTA |
| | | acid | GAAACTATTTAAATTGGTATCAGCAGAAACCAGGGAAAGCCCCT |
| | | sequence | AAGCTCCTGATCTACGATGCATCCAATTTGGAAACAGGGGTCCC |
| | | | ATCGAGGTTCAGTGGAAGTGGATCTGGGACAGATTTTACTTTCA |
| | | | CCATCAGCAGCCTGCAGCCTGAAGATATTGCAACATATTACTGT |
| | | | CAACAGTATGATAAGCTCCTTTTCATTTTCGGCCCTGGGACCAA |
| | | | AGTGGATATCAAA |
| 688 | YANG-1203 | VL domain | DIQMTQSPSSLSASVGDRVTITCQASQDIRNYLNWYQQKPGKAP |
| | | amino | KLLIYDASNLETGVPSRFSGSGSGTDFTFTISSLQPEDIATYYC |
| | | acid | QQYDKLLFIFGPGTKVDIK |
| | | sequence | |
| 689 | YANG-1203 | LCDR1 | QDIRNY |
| 28 | YANG-1203 | LCDR2 | DAS |
| 690 | YANG-1203 | LCDR3 | QQYDKLLFI |
| 691 | YANG-1204 | VH domain | CAGGTGCAGCTGGTGGAGTCTGGGGGAGGCGTGGTCCAGCCTGG |
| | | nucleic | GAGGTCCCTGAGACTCTCCTGTGCAGTCTCTGGATTCACCTTCA |
| | | acid | GTAACTATGGCATACACTGGGTCCGCCAGGCTCCAGGCAAGGGG |
| | | sequence | CTGGTGTGGGTGGCAGTTATATCATATGAAGGAAGTATTAAATA |
| | | | TTATGGAGACTCCGTGAAGGGCCGATTCACCATCTCCAGAGACA |
| | | | ATTCCAAGAACACGCTGTATCTGCAAATGAACAGCCTGAGAGCC |
| | | | GAGGACACGGCTGTGTATTACTGTGCGAGAGGGGCAGTGGCTGG |
| | | | CAATGATGCTTTTGATATCTGGGGCCAAGGTACAATGGTCACCG |
| | | | TCTCTTCA |
| 692 | YANG-1204 | VH domain | QVQLVESGGGVVQPGRSLRLSCAVSGFTFSNYGIHWVRQAPGKG |
| | | amino | LVWVAVISYEGSIKYYGDSVKGRFTISRDNSKNTLYLQMNSLRA |
| | | acid | EDTAVYYCARGAVAGNDAFDIWGQGTMVTVSS |
| | | sequence | |
| 693 | YANG-1204 | HCDR1 | GFTFSNYG |
| 694 | YANG-1204 | HCDR2 | ISYEGSIK |
| 695 | YANG-1204 | HCDR3 | ARGAVAGNDAFDI |
| 696 | YANG-1204 | VL domain | GATGTTGTGATGACTCAGTCTCCACTCTCCCTGCCCGTCACCCT |
| | | nucleic | TGGACAGCCGGCCTCCATCTCCTGCAGGTCTAGTCAAAGCCTCG |
| | | acid | TATACAGTGATGGAAACACCTACTTGATTTGGTTTCAGCAGAGG |
| | | sequence | CCAGGCCAATCTCCAAGGCGCCTAATTTATAAGGTTTCTAATCG |
| | | | GGACTCTGGGGTCCCAGACAGATTCAGCGGCGGTGGGTCAGGCA |
| | | | CTGATTTCACACTGAAAATCAGCAGGGTGGAGGCTGAGGATGTT |
| | | | GGGGTTTATTACTGCATGCAAGGTACACACTGGCCTCTCACTTT |
| | | | CGGCGGAGGGACCAAGGTGGAGATCAAA |
| 697 | YANG-1204 | VL domain | DVVMTQSPLSLPVTLGQPASISCRSSQSLVYSDGNTYLIWFQQR |
| | | amino | PGQSPRRLIYKVSNRDSGVPDRFSGGGSGTDFTLKISRVEAEDV |
| | | acid | GVYYCMQGTHWPLTFGGGTKVEIK |
| | | sequence | |
| 698 | YANG-1204 | LCDR1 | QSLVYSDGNTY |
| 85 | YANG-1204 | LCDR2 | KVS |
| 699 | YANG-1204 | LCDR3 | MQGTHWPLT |
| 700 | YANG-1205 | VH domain | CAGGTGCAGCTGGTGCAGTCTGGGGCTGAGGTGAAGAAGCCTGG |
| | | nucleic | GGCCTCAGTGAAGGTTTCCTGCAAGGCATCTGGATACACCTTCA |
| | | acid | CCAGCTACTGTATACACTGGGTGCGACAGGCCCCTGGACAAGGA |
| | | sequence | CTTGAGTGGATGGGAATAATCAACCCTAGTGGTGGTGGCACAAT |
| | | | CTTCGCACAGAAGTTCCAGGGCAGAGTCACCATGACCAGGGACA |
| | | | CGTCCACGACCACAGTCTACATGGAGTTGGGCAGCCTGAGATCT |
| | | | GACGACACGGCCCTGTATTACTGTGCGCGAGGGTGGTCTTACGA |
| | | | TTTTTGGAGTGGCCCTGACTACTGGGGCCAGGGAACCCTGGTCT |
| | | | CCGTCTCCTCT |
| 701 | YANG-1205 | VH domain | QVQLVQSGAEVKKPGASVKVSCKASGYTFTSYCIHWVRQAPGQG |
| | | amino | LEWMGIINPSGGGTIFAQKFQGRVTMTRDTSTTTVYMELGSLRS |
| | | acid | DDTALYYCARGWSYDFWSGPDYWGQGTLVSVSS |
| | | sequence | |
| 702 | YANG-1205 | HCDR1 | GYTFTSYC |
| 703 | YANG-1205 | HCDR2 | INPSGGGT |
| 704 | YANG-1205 | HCDR3 | ARGWSYDFWSGPDY |
| 705 | YANG-1205 | VL domain | GATATTGTGATGACCCAGACTCCACTCTCTCTGTCCGTCACCCC |
| | | nucleic | TGGACAGCCGGCCTCCATCTCCTGCAAGTCTAGTCAGAGCCTCC |
| | | acid | TGCATAGTGATGGAAAGACCTATTTGTATTGGTACCTGCAGAAG |
| | | sequence | CCAGGCCAGCCTCCACAGCTCCTGATCTATGAAGTTTCCAACCG |
| | | | GTTCTCTGGAGTGCCAGATAGGTTCAGTGGCAGCGGGTCAGGGA |
| | | | CAGATTTCACACTGAAAATCAGCCGGGTGGGGGCTGAGGATGTT |
| | | | GGGATTTATTATTGCATGCACAGTATAAAGCTTCCTCCGACGTT |
| | | | CGGCCAAGGGACCAAGGTGGAAATCAAA |
| 706 | YANG-1205 | VL domain | DIVMTQTPLSLSVTPGQPASISCKSSQSLLHSDGKTYLYWYLQK |
| | | amino | PGQPPQLLIYEVSNRFSGVPDRFSGSGSGTDFTLKISRVGAEDV |
| | | acid | GIYYCMHSIKLPPTFGQGTKVEIK |
| | | sequence | |
| 707 | YANG-1205 | LCDR1 | QSLLHSDGKTY |
| 615 | YANG-1205 | LCDR2 | EVS |
| 708 | YANG-1205 | LCDR3 | MHSIKLPPT |
| 709 | YANG-1206 | VH domain | CAGGTGCACCTGGTGGAGTCTGGGGGAGGCATGGTCCAGCCTGG |
| | | nucleic | GAGGTCCCTGAGACTCTCCTGTGCAGCCTCTGGATTCACCTTCA |
| | | acid | GTATCTATGCCATGCACTGGGTCCGCCAGGCTCCAGGCAAGGGG |
| | | sequence | CTGGAGTGGGTGGCAGTTATATCATATGATGGAAGTAATAAATA |
| | | | TTATGCAGACTCCGTGAAGGGCCGATTCACCATCTCCAGAGACA |
| | | | ATTCCAAGAACACGCTGTATCTGCAAATGAACAGCCTGAGAGCT |
| | | | GAGGACACGGCTGTGTATTACTGTGCGAAAGTCTATGGTGGGAG |
| | | | CTACTGGGGGGGCTTTGACTACTGGGGCCAGGGAACCCTGGTCA |
| | | | CCGTCTCCTCA |
| 710 | YANG-1206 | VH domain | QVHLVESGGGMVQPGRSLRLSCAASGFTFSIYAMHWVRQAPGKG |
| | | amino | LEWVAVISYDGSNKYYADSVKGRFTISRDNSKNTLYLQMNSLRA |
| | | acid | EDTAVYYCAKVYGGSYWGGFDYWGQGTLVTVSS |
| | | sequence | |
| 711 | YANG-1206 | HCDR1 | GFTFSIYA |
| 712 | YANG-1206 | HCDR2 | ISYDGSNK |
| 713 | YANG-1206 | HCDR3 | AKVYGGSYWGGFDY |
| 714 | YANG-1206 | VL domain | CAGTCTGTGCTGACGCAGCCGCCCTCAGTGTCTGGGGCCCCAGG |
| | | nucleic | GCAGAGGGTCACCATCTCCTGCACTGGGACCAGCTCCAACATCG |
| | | acid | GGTCAATTTATGATGTACACTGGTACCAGCAGCTTCCAGGAACA |
| | | sequence | GCCCCCAAACTCCTCATCTATGGTAACAGCAATCGGCCCTCAGG |
| | | | GGTCCCTGACCGATTCTCTGGCTCCAAGTCTGGCACCTCGGCCT |
| | | | CCCTGGCCATCACTGGGCTCCAGGCTGAGGATGAGGCTGATTAT |
| | | | TACTGCCAGTCCTATGACACCAGCCTGAGTGGTTCTTGGGTGTT |
| | | | CGGCGGAGGGACCAAGCTGAACGTCCTA |
| 715 | YANG-1206 | VL domain | QSVLTQPPSVSGAPGQRVTISCTGTSSNIGSIYDVHWYQQLPGT |
| | | amino | APKLLIYGNSNRPSGVPDRFSGSKSGTSASLAITGLQAEDEADY |
| | | acid | YCQSYDTSLSGSWVFGGGTKLNVL |
| | | sequence | |
| 716 | YANG-1206 | LCDR1 | SSNIGSIYD |
| 717 | YANG-1206 | LCDR2 | GNS |
| 718 | YANG-1206 | LCDR3 | QSYDTSLSGSWV |
| 719 | YANG-1207 | VH domain | CAGGTGCAGCTGGTGGAGTCTGGGGGAGGCGTGGTCCAGCCTGG |
| | | nucleic | GAGGTCCCTGAGACTCTCCTGTGCAGCGTCTGGATTCACCTTCA |
| | | acid | GTAGTTATGGCATGCACTGGGTCCGCCAGGCTCCAGGCAAGGGG |
| | | sequence | CTGGAGTGGGTGTCAGTTATATGGTATGATGGAACTAATAAATA |
| | | | CTATGCAGACTCCGTGAAGGGCCGATTCACCATCTCCAGAGACA |
| | | | ATTCCAAGAACACGCTGTATCTGCAAATGAACAGCCTGAGAGCC |
| | | | GAGGACACGGCTGTGTATTACTGTGCGAGAGAGGGAGTGGAATT |
| | | | CACTGGGTACTTTGACTACTGGGGCCAGGGAACCCTGGTCACCG |
| | | | TCTCCTCA |
| 720 | YANG-1207 | VH domain | QVQLVESGGGVVQPGRSLRLSCAASGFTFSSYGMHWVRQAPGKG |
| | | amino | LEWVSVIWYDGINKYYADSVKGRFTISRDNSKNTLYLQMNSLRA |
| | | acid | EDTAVYYCAREGVEFTGYFDYWGQGTLVTVSS |
| | | sequence | |
| 721 | YANG-1207 | HCDR1 | GFTFSSYG |
| 722 | YANG-1207 | HCDR2 | IWYDGTNK |
| 723 | YANG-1207 | HCDR3 | AREGVEFTGYFDY |
| 724 | YANG-1207 | VL domain | TCCTATGAGCTGACTCAGCCACCCTCAGTGTCCGTGTCCCCAGG |
| | | nucleic | ACAGACAGCCAGCATCACCTGCTCTGGAGATAAATTGGGGGATA |
| | | acid | AATATGCTTGCTGGTATCAGCAGAAGCCAGGCCAGTCCCCTGTG |
| | | sequence | CTGGTCATCTATCAAGATAGCAAGCGGCCCTCAGGGATCCCTGA |
| | | | GCGATTCTCTGGCTCCAACTCTGGGAACACAGCCACTCTGACCA |
| | | | TCAGCGGGACCCAGGCTATGGATGAGGCTGACTATTACTGTCAG |
| | | | GCGTGGACCAGCAACACTGCAGTGGTATTCGGCGGAGGGACCAA |
| | | | GCTGACCGTCCTA |
| 725 | YANG-1207 | VL domain | SYELTQPPSVSVSPGQTASITCSGDKLGDKYACWYQQKPGQSPV |
| | | amino | LVIYQDSKRPSGIPERFSGSNSGNTATLTISGTQAMDEADYYCQ |
| | | acid | AWTSNTAVVFGGGTKLTVL |
| | | sequence | |
| 726 | YANG-1207 | LCDR1 | KLGDKY |
| 727 | YANG-1207 | LCDR2 | QDS |
| 728 | YANG-1207 | LCDR3 | QAWTSNTAVV |
| 729 | YANG-1301 | VH domain | CAGGTGCAGCTCGTGGAGTCTGGGGGAGGCGTGGTCCAGCCTGG |
| | | nucleic | GAGGTCCCTGAGACTCTCCTGTGCAGCGTCTGGATTCACCTTCA |
| | | acid | GTAGTTATGGCATGCACTGGGTCCGCCAGGCTCCAGGCAAGGGG |
| | | sequence | CTGGAGTGGGTGGCAGTTATCTGGTATGATGGAAGTAATAAATT |
| | | | CTATGCAGACTCCGTGAAGGGCCGATTCACCATCTCCAGAGACA |
| | | | ATTCCAAGAACACACTGTATCTGCAAATGAACAGCCTAAAAGCC |
| | | | GAAGACACGGCTGTGTATTACTGTGCGAGAGATACCTGGATCGG |
| | | | GGAGTCCGATACTAGCTGGCTCGACCCCTGGGGCCAGGGAACCC |
| | | | TGGTCACCGTCTCCTCA |
| 730 | YANG-1301 | VH domain | QVQLVESGGGVVQPGRSLRLSCAASGFTFSSYGMHWVRQAPGKG |
| | | amino | LEWVAVIWYDGSNKFYADSVKGRFTISRDNSKNTLYLQMNSLKA |
| | | acid | EDTAVYYCARDTWIGESDTSWLDPWGQGTLVTVSS |
| | | sequence | |
| 731 | YANG-1301 | HCDR1 | GFTFSSYG |
| 732 | YANG-1301 | HCDR2 | IWYDGSNK |
| 733 | YANG-1301 | HCDR3 | ARDTWIGESDTSWLDP |
| 734 | YANG-1301 | VL domain | TCCTATGAGCTGACTCAGCCACCCTCAGTGTCCGTGTCCCCGGG |
| | | nucleic | ACAGACAGCCAGCATCACCTGCTCTGGAGATAAATTGGGGGATA |
| | | acid | AATATGCTTGCTGGTATCAACAGAAGCCAGGCCAGTCCCCTGTA |
| | | sequence | TTAATCGTCTATCAAGATAGCAAGCGGCCCTCAGGGATCCCTGA |
| | | | GCGATTCTCTGGCTCCAACTCTGGGAACACAGCCACTCTGACCA |
| | | | TCAGCGGGACCCAGGCTATGGATGAGGCTGATTATTACTGTCAG |
| | | | GCGTGGGACAGCTTCACTGCCGTGGTATTCGGCGGAGGGACCAA |
| | | | GCTGACCGTCCTA |
| 735 | YANG-1301 | VL domain | SYELTQPPSVSVSPGQTASITCSGDKLGDKYACWYQQKPGQSPV |
| | | amino | LIVYQDSKRPSGIPERFSGSNSGNTATLTISGTQAMDEADYYCQ |
| | | acid | AWDSFTAVVFGGGTKLTVL |
| | | sequence | |
| 736 | YANG-1301 | LCDR1 | KLGDKY |
| 727 | YANG-1301 | LCDR2 | QDS |
| 737 | YANG-1301 | LCDR3 | QAWDSFTAVV |
| 738 | YANG-1302 | VH domain | CAGGTGCAGCTGGTGGAGTCTGGGGGAGGCGCGGTCCAGCCTGG |
| | | nucleic | GAGGTCCCTGAGACTCTCCTGTAGAGCGTCTGGATTCACCTTCA |
| | | acid | GTAGTTTTGGCATGAATTGGGTCCGCCAGGTTCCAGGCAAGGGG |
| | | sequence | CTGGTATGGGTGGCAGGTATATGGTATGATGGAAGGAATAAGTA |
| | | | CTATGCAGACTCCGTGAAGGGCCGATTCACCATCTCCAGAGAGA |
| | | | ATTCCAAGAATATGCTGTATCTGCAAATGAACAGTCTGAGAGCC |
| | | | GAGGACACGGCTGTGTATTACTGTGCGAGAGATCAAGATAGTGG |
| | | | TTTCGATGGCAACTGGTTCGGCCCCTGGGGCCAGGGAACCATGG |
| | | | TCACCGTCTCCTCA |
| 739 | YANG-1302 | VH domain | QVQLVESGGGAVQPGRSLRLSCRASGFTFSSFGMNWVRQVPGKG |
| | | amino | LVWVAGIWYDGRNKYYADSVKGRFTISRENSKNMLYLQMNSLRA |
| | | acid | EDTAVYYCARDQDSGFDGNWFGPWGQGTMVTVSS |
| | | sequence | |
| 740 | YANG-1302 | HCDR1 | GFTFSSFG |
| 741 | YANG-1302 | HCDR2 | IWYDGRNK |
| 742 | YANG-1302 | HCDR3 | ARDQDSGFDGNWFGP |
| 743 | YANG-1302 | VL domain | TCCTATGAACTGACTCAGCCACCCTCAATGTCCGTGTCCCCAGG |
| | | nucleic | ACAGACAGCCAGCATCACCTGCTCTGGAGATAACTTGGGGGATA |
| | | acid | AATATGTTTGCTGGTATCAACAGAGGCCAGGCCAGTCCCCTGTG |
| | | sequence | ATGGTCATCTTTCAAGATAGCACGCGGCCCTCAGGGATCCCTGA |
| | | | GCGATTCTCTGGCTCCAACTCTGGAAACACAGCCACTCTGACCA |
| | | | TCAGCGGGACCCAGGCTATGGATGAGGCTGACTATTACTGTCAG |
| | | | GCGTGGGACAGCAACACTGCGGTATTCGGCGGAGGGACCAAGCT |
| | | | GACCGTCCTA |
| 744 | YANG-1302 | VL domain | SYELTQPPSMSVSPGQTASITCSGDNLGDKYVCWYQQRPGQSPV |
| | | amino | MVIFQDSTRPSGIPERFSGSNSGNTATLTISGTQAMDEADYYCQ |
| | | acid | AWDSNTAVFGGGTKLTVL |
| | | sequence | |
| 745 | YANG-1302 | LCDR1 | NLGDKY |
| 727 | YANG-1302 | LCDR2 | QDS |
| 746 | YANG-1302 | LCDR3 | QAWDSNTAV |
| 747 | YANG-1303 | VH domain | CAGGTTCAGCTGGTGCAGTCTGGAACTGAGATGAAGGAGCCTGG |
| | | nucleic | GGCCTCAGTGAAGGTCTCCTGCAAGGCTTCTGGTTACACCTTTA |
| | | acid | CCAACTATGGTATCACCTGGGTGCGACAGGCCCCTGGACAAGGG |
| | | sequence | CTTGAGTGGATGGGATGGCTCAACACTAACAATGGGGACACAAA |
| | | | CTATGCTCAGAAACTCCAGGGCAGAGTCACCATGACCACAGACA |
| | | | CATCCACGAGCACAGCCTACATGGAACTGAGGAGCCTGAGATCT |
| | | | GACGACACGGCCGTGTATTATTGTGCGCGAGACTCGGTGACTAC |
| | | | GTATGACTACTGGGGCCAGGGAACCCTGGTCACCGTCTCCTCA |
| 748 | YANG-1303 | VH domain | QVQLVQSGTEMKEPGASVKVSCKASGYTFTNYGITWVRQAPGQG |
| | | amino | LEWMGWLNTNNGDTNYAQKLQGRVTMTTDTSTSTAYMELRSLRS |
| | | acid | DDTAVYYCARDSVTTYDYWGQGTLVTVSS |
| | | sequence | |
| 749 | YANG-1303 | HCDR1 | GYTFTNYG |
| 750 | YANG-1303 | HCDR2 | LNTNNGDT |
| 751 | YANG-1303 | HCDR3 | ARDSVTTYDY |
| 752 | YANG-1303 | VL domain | TCCTATGAACTGACACAGCCACCCTCGGTGTCAGTGTCCCCAGG |
| | | nucleic | ACAAACGGCCAGGATCACCTGCTCTGGAGATGCATTGCCAAAAA |
| | | acid | AATATGCTTATTGGTTCCAGCAGAAGTCAGCCCAGGCCCCTGTG |
| | | sequence | CTGGTCATCTATGAGGACAGCAAACGACCCTCCGGGATCCCTGA |
| | | | GAGATTCTCTGGCTCCAGCTCAGGGACAATGGCCACCTTGACTA |
| | | | TCAATGGGGCCCAGGTGGAGGATGAAGCTGCCTACTACTGTTAT |
| | | | TCATTGGACAGCAGTGGTAATCATTGGGTGTTCGGCGGAGGGAC |
| | | | CAAGTTGACCGTCCTA |
| 753 | YANG-1303 | VL domain | SYELTQPPSVSVSPGQTARITCSGDALPKKYAYWFQQKSAQAPV |
| | | amino | LVIYEDSKRPSGIPERFSGSSSGTMATLTINGAQVEDEAAYYCY |
| | | acid | SLDSSGNHWVFGGGTKLTVL |
| | | sequence | |
| 754 | YANG-1303 | LCDR1 | ALPKKY |
| 755 | YANG-1303 | LCDR2 | EDS |
| 756 | YANG-1303 | LCDR3 | YSLDSSGNHWV |
| 757 | YANG-1304 | VH domain | CAGGTTCAACTACAGCAGTGGGGCGCAGGACTGGTGAAGACTTC |
| | | nucleic | GGAGACCCTGTCCCTCACCTGCGCTGTCTATGGTGGGTCTTTCA |
| | | acid | ATGATCACTATTGGAGCTGGATCCGACAGCCCCCAGGAAAGGGA |
| | | sequence | CTGGAGTGGATTGGGGAAATCAATCATAGTGGAAGCACCAACTA |
| | | | CAATCCGTCCCTCAAGAGTCGAGTCACCATGTCACTGGACACGT |
| | | | CCAAGAACCAGTTCTCCCTCAAGTTGAGGTCTTTGACCGCCGCG |
| | | | GACACGGCTATGTATTACTGTGCGATTGAGGGAATCTGGGGCCA |
| | | | GGGAGCCATGGTCACCGTCTCCTCA |
| 758 | YANG-1304 | VH domain | QVQLQQWGAGLVKTSETLSLTCAVYGGSENDHYWSWIRQPPGKG |
| | | amino | LEWIGEINHSGSTNYNPSLKSRVTMSLDTSKNQFSLKLRSLTAA |
| | | acid | DTAMYYCAIEGIWGQGAMVTVSS |
| | | sequence | |
| 759 | YANG-1304 | HCDR1 | GGSENDHY |
| 760 | YANG-1304 | HCDR2 | INHSGST |
| 761 | YANG-1304 | HCDR3 | AIEGI |
| 762 | YANG-1304 | VL domain | TCCTATGAGCTGACACAGCCACCCTCGGTGTCAGTGTCCCCAGG |
| | | nucleic | ACAAACGGCCAGGATCACCTGCTCTGGAGATGCATTGCCAATTA |
| | | acid | AATATGTTCATTGGTACCAGCAGAAGTCAGGCCAGGCCCCTGTG |
| | | sequence | CTGGTCATCTATGAGGACAGCAAACGACCCTCCGGGATCCCTGA |
| | | | GAGAATCTCTGGCTCCAGCTCAGGGACAATGGCCACCTTGACTA |
| | | | TGAGTGGGGCCCAGGTGGAGGATGAAGCTGACTACTACTGTTAC |
| | | | TCAACAGACAGCAGTGGTAATCATTGGGTGTTCGGCGGAGGGAC |
| | | | CAAGCTGACCGTCCTA |
| 763 | YANG-1304 | VL domain | SYELTQPPSVSVSPGQTARITCSGDALPIKYVHWYQQKSGQAPV |
| | | amino | LVIYEDSKRPSGIPERISGSSSGTMATLTMSGAQVEDEADYYCY |
| | | acid | STDSSGNHWVFGGGTKLTVL |
| | | sequence | |
| 764 | YANG-1304 | LCDR1 | ALPIKY |
| 755 | YANG-1304 | LCDR2 | EDS |
| 765 | YANG-1304 | LCDR3 | YSTDSSGNHWV |
| 766 | YANG-1305 | VH domain | CAGGTGCAGCTGGTGGAGTCTGGGGGAGGCGTGGTCCAGCCTGG |
| | | nucleic | GAAGTCCCTGAGACTCTCCTGTGCAGCCTCTGGATTCGCCTTCA |
| | | acid | GTAGCTTTGGCATGCACTGGGTCCGCCAGGCTCCAGGCACGGGG |
| | | sequence | CTGGAGTGGTTGGCAATTATATCATTTGAAGGAACTAAAAAATA |
| | | | CTATGCAGACTCCATGAAGGGCCGAATCACCATCTCCAGAGACA |
| | | | ATTCCAAGAACACGCTGTATCTGCAAATGAACAGTTTGAGAGCT |
| | | | GAGGACACGGCTGTGTATTACTGTGCGGCTGACTATGGTGACTA |
| | | | CGACGACTATTACTACGGTGTGCACGTCTGGGGCCAAGGGACCA |
| | | | CGGTCACCGTCTCCTCA |
| 767 | YANG-1305 | VH domain | QVQLVESGGGVVQPGKSLRLSCAASGFAFSSFGMHWVRQAPGTG |
| | | amino | LEWLAIISFEGTKKYYADSMKGRITISRDNSKNTLYLQMNSLRA |
| | | acid | EDTAVYYCAADYGDYDDYYYGVHVWGQGTTVTVSS |
| | | sequence | |
| 768 | YANG-1305 | HCDR1 | GFAFSSFG |
| 769 | YANG-1305 | HCDR2 | ISFEGTKK |
| 770 | YANG-1305 | HCDR3 | AADYGDYDDYYYGVHV |
| 771 | YANG-1305 | VL domain | GACATCCAAATGACCCAGTCTCCATCCTCCCTGTCTGCATCTGT |
| | | nucleic | AGGAGACAGAGTCACCATCACTTGCCGGGCGAGTCAGGGCATTG |
| | | acid | CCAATTATTTAGCCTGGTATCAGCAGAAACCGGGGAAAGTTCCT |
| | | sequence | AAGCTCCTGATCTATGCTACATCCACTTTGCAATCAGGGGTCCC |
| | | | ATCTCGGTTCAGTGGCAGTGGATCTGGGACAGATTTCACTCTCA |
| | | | CCATCAGCAGCCTGCAGCCTGAAGATGTTGCAATTTATTACTGT |
| | | | CAAAAGTATGACAGTGCCCCATTCACTTTCGGCCCTGGGACCAA |
| | | | AGTGGATTTCAAA |
| 772 | YANG-1305 | VL domain | DIQMTQSPSSLSASVGDRVTITCRASQGIANYLAWYQQKPGKVP |
| | | amino | KLLIYATSTLQSGVPSRFSGSGSGTDFTLTISSLQPEDVAIYYC |
| | | acid | QKYDSAPFTFGPGTKVDFK |
| | | sequence | |
| 773 | YANG-1305 | LCDR1 | QGIANY |
| 774 | YANG-1305 | LCDR2 | ATS |
| 775 | YANG-1305 | LCDR3 | QKYDSAPFT |
| 776 | YANG-1401 | VH domain | CAGGTGCACCTGCAGGAGTCGGGCCCAGGACTGGTGAAGCCTTC |
| | | nucleic | GGGGACCCTGTCCCTCACCTGCGCTGTCTCTGGTGGCTCCTTCA |
| | | acid | GCCAGATTAACTGGTGGAGTTGGGTCCGCCAGTCCCCAGGAAAG |
| | | sequence | GGGCTGGAGTGGATTGGGGAAATCTATCATAGTGGGAGTACCAA |
| | | | CTACAACCCGTCCCTCAAGAGTCGAGTCACCATGTCAGTAGACA |
| | | | AGTCCAAGAACCAGTTCTCCCTGGCGCTGAACTCTGTGACCGCC |
| | | | GCGGACACGGCCGTATATTACTGTGCGAGGGGGTATTATGGTGA |
| | | | CAACTGGTTCGACTCCTGGGGCCAGGGAACCCTGGTCACCGTCT |
| | | | CCTCA |
| 777 | YANG-1401 | VH domain | QVHLQESGPGLVKPSGTLSLTCAVSGGSFSQINWWSWVRQSPGK |
| | | amino | GLEWIGEIYHSGSTNYNPSLKSRVTMSVDKSKNQFSLALNSVTA |
| | | acid | ADTAVYYCARGYYGDNWFDSWGQGTLVTVSS |
| | | sequence | |
| 778 | YANG-1401 | HCDR1 | GGSFSQINW |
| 779 | YANG-1401 | HCDR2 | IYHSGST |
| 780 | YANG-1401 | HCDR3 | ARGYYGDNWEDS |
| 781 | YANG-1401 | VL domain | GACATCCAGATGACCCAGTCTCCATCCTCACTGTCTGCTTCTAT |
| | | nucleic | AGGCGACAGAGTCACCATCACTTGTCGGGCGAGTCAGGGTATTA |
| | | acid | GCAACTGGTTAGTCTGGTATCAGCAGAAACCAGAGAAAGCCCCT |
| | | sequence | AAGTCCCTGATCTATGCTGCATCCAGTTTGCAAAGTGGGGTCCC |
| | | | ATCAAGGTTCAGCGGCAGTGGATCTGGGACAGATTTCACTCTAA |
| | | | CCATCAGCAGCCTGCATCCTGAAGATTTTGCAACTTATTACTGC |
| | | | CAACAGTATAGTGATTACCCTCTCACTTTCGGCGGAGGGACCAA |
| | | | GGTGGAGATCAAA |
| 182 | YANG-1401 | VL domain | DIQMTQSPSSLSASIGDRVTITCRASQGISNWLVWYQQKPEKAP |
| | | amino | KSLIYAASSLQSGVPSRFSGSGSGTDFTLTISSLHPEDFATYYC |
| | | acid | QQYSDYPLTFGGGTKVEIK |
| | | sequence | |
| 783 | YANG-1401 | LCDR1 | QGISNW |
| 18 | YANG-1401 | LCDR2 | AAS |
| 784 | YANG-1401 | LCDR3 | QQYSDYPLT |
| 785 | YANG-1401a | VH domain | CAGGTGCACCTGCAGGAGTCGGGCCCAGGACTGGTGAAGCCTTC |
| | | nucleic | GGGGACCCTGTCCCTCACCTGCGCTGTCTCTGGTGGCTCCTTCA |
| | | acid | GCCAGATTAACTGGTGGAGTTGGGTCCGCCAGTCCCCAGGAAAG |
| | | sequence | GGGCTGGAGTGGATTGGGGAAATCTATCATAGTGGGAACACCAA |
| | | | CTACAACCCGTCCCTCAAGAGTCGAGTCACCATGTCAGTAGACA |
| | | | AGTCCAAGAACCAGTTCTCCCTGGCGCTGAACTCTGTGACCGCC |
| | | | GCGGACACGGCCGTGTATTATTGTGCGAGGGGGTATTATGGTGA |
| | | | CAACTGGTTCGACTCCTGGGGCCAGGGAACCCTGGTCACCGTCT |
| | | | CCTCA |
| 786 | YANG-1401a | VH domain | QVHLQESGPGLVKPSGTLSLTCAVSGGSFSQINWWSWVRQSPGK |
| | | amino | GLEWIGEIYHSGNTNYNPSLKSRVTMSVDKSKNQFSLALNSVTA |
| | | acid | ADTAVYYCARGYYGDNWFDSWGQGTLVTVSS |
| | | sequence | |
| 787 | YANG-1401a | HCDR1 | GGSFSQINW |
| 788 | YANG-1401a | HCDR2 | IYHSGNT |
| 789 | YANG-1401a | HCDR3 | ARGYYGDNWEDS |
| 790 | YANG-1401a | VL domain | GACATCCAGATGACCCAGTCTCCATCCTCACTGTCTGCTTCTAT |
| | | nucleic | AGGCGACAGAGTCACCATCACTTGTCGGGCGAGTCAGGGTATTA |
| | | acid | GCAGCTGGTTAGTCTGGTATCAGCAGAAACCAGAGAAAGCCCCT |
| | | sequence | AAGTCCCTGATCTATGCTGCATCCAGTTTGCAAAGTGGGGTCCC |
| | | | ATCAAGGTTCAGCGGCAGTGGATCTGGGACAGATTTCACTCTCA |
| | | | CCATCAGCAGCCTGCATCCTGAAGATTTTGCAACTTATTACTGC |
| | | | CAACAGTATAGTGATTACCCTCTCACTTTCGGCGGAGGGACCAA |
| | | | GGTGGAGATCAAA |
| 791 | YANG-1401a | VL domain | DIQMTQSPSSLSASIGDRVTITCRASQGISSWLVWYQQKPEKAP |
| | | amino | KSLIYAASSLQSGVPSRFSGSGSGTDFTLTISSLHPEDFATYYC |
| | | acid | QQYSDYPLTFGGGTKVEIK |
| | | sequence | |
| 792 | YANG-1401a | LCDR1 | QGISSW |
| 18 | YANG-1401a | LCDR2 | AAS |
| 793 | YANG-1401a | LCDR3 | QQYSDYPLT |
| 794 | YANG-1401b | VH domain | CAGGTGCAGCTGCAGGAGTCGGGCCCAGGACTGGTGAAGCCTTC |
| | | nucleic | GGGGACCCTGTCCCTCACCTGCGTTGTCTCTGGTGGCTCCTTCA |
| | | acid | GCAATACTAATTGGTGGAGTTGGGTCCGCCAGCCCCCAGAAAAG |
| | | sequence | GGGCTGGAGTGGATTGGGGAAGTCTATCATAGTGGGAGCACCAA |
| | | | CTACAACCCGTCCCTCATGAATCGAGTCACCATATCAGTAGACA |
| | | | AGTCCAGGAACCAGTTCTCCCTGAACCTGAGTTCTGTGACCGCC |
| | | | GCGGACACGGCCGTGTATTTCTGTGCGAGGGGGTATTATGGTGA |
| | | | CAATTGGTTCGACTCCTGGGGCCAGGGAACCCTGGTCACCGTCT |
| | | | CCTCA |
| 795 | YANG-1401b | VH domain | QVQLQESGPGLVKPSGTLSLTCVVSGGSFSNTNWWSWVRQPPEK |
| | | amino | GLEWIGEVYHSGSTNYNPSLMNRVTISVDKSRNQFSLNLSSVTA |
| | | acid | ADTAVYFCARGYYGDNWEDSWGQGTLVTVSS |
| | | sequence | |
| 796 | YANG-1401b | HCDR1 | GGSFSNTNW |
| 797 | YANG-1401b | HCDR2 | VYHSGST |
| 798 | YANG-1401b | HCDR3 | ARGYYGDNWFDS |
| 799 | YANG-1401b | VL domain | GACATCCAGATGACCCAGTCTCCATCCTCACTGTCTGCATCTGT |
| | | nucleic | AGGAGACAGAGTCATCATCACTTGTCGGGCGAGTCAGGGTATTA |
| | | acid | GCAGTTGGTTAGCCTGGTATCAGCAGAAACCAGAGAAAGCCCCT |
| | | sequence | AAGTCCCTGATCTATTCTGCATCCACTTTGCAAAGTGGAGTCCC |
| | | | ATCAAGGTTCAGCGGCAGTGGATCTGGGACAGATTTCACTCTCA |
| | | | CCATCAGCAGCCTGCAGCCTGAAGATTTTGCAACTTATTACTGC |
| | | | CAACAGTCTAATAGTTACCCTCTCACTTTCGGCGGAGGGACCAA |
| | | | GGTCGAGATCAAA |
| 800 | YANG-1401b | VL domain | DIQMTQSPSSLSASVGDRVIITCRASQGISSWLAWYQQKPEKAP |
| | | amino | KSLIYSASTLQSGVPSRFSGSGSGTDFTLTISSLQPEDFATYYC |
| | | acid | QQSNSYPLTFGGGTKVEIK |
| | | sequence | |
| 801 | YANG-1401b | LCDR1 | QGISSW |
| 517 | YANG-1401b | LCDR2 | SAS |
| 802 | YANG-1401b | LCDR3 | QQSNSYPLT |
| 803 | YANG-1401c | VH domain | CAGGTGCAGCTGCAGGAGTCGGGCCCAGGACTGGTGAAGCCTTC |
| | | nucleic | GGGGACCCTGTCCCTCACCTGCGTTGTCTCTGGTGGCTCCTTCA |
| | | acid | CCAATACTAATTGGTGGAGTTGGGTCCGCCAGCCCCCAGGGAAG |
| | | sequence | GGGCTGGAGTGGATTGGGGAAGTCTATCATAGTGGGAGCACCAA |
| | | | CTACAACCCGTCCCTCAAGAGTCGAGTCACCATATCAGTAGACA |
| | | | AGTCCAAGAACCAGTTCTCCCTGAACCTGACTTCTGTGACCGCC |
| | | | GCGGACACGGCCGTGTATTACTGTGCGAGGGGGTATTATGGTGA |
| | | | CAATTGGTTCGACTCCTGGGGCCAGGGAACCCTGGTCACCGTCT |
| | | | CCTCA |
| 804 | YANG-1401c | VH domain | QVQLQESGPGLVKPSGTLSLTCVVSGGSFTNTNWWSWVRQPPGK |
| | | amino | GLEWIGEVYHSGSTNYNPSLKSRVTISVDKSKNQFSLNLTSVTA |
| | | acid | ADTAVYYCARGYYGDNWFDSWGQGTLVTVSS |
| | | sequence | |
| 805 | YANG-1401c | HCDR1 | GGSFTNTNW |
| 806 | YANG-1401c | HCDR2 | VYHSGST |
| 807 | YANG-1401c | HCDR3 | ARGYYGDNWEDS |
| 808 | YANG-1401c | VL domain | GACATCCAGATGACCCAGTCTCCATCCTCACTGTCTGCATCTGT |
| | | nucleic | AGGAGACAGAGTCACCATCACTTGTCGGGCGAGTCAGGGTATTA |
| | | acid | GCAGCTGGTTAGCCTGGTATCAGCAGAAACCAGAGAAAGCCCCT |
| | | sequence | AAGTCCCTGATCTATTCTGCATCCACTTTGCAAAGTGGAGTCCC |
| | | | ATCAAGGTTCAGCGGCAGTGGATCTGGGACAGATTTCACTCTCA |
| | | | CCATCAGCAGCCTGCAGCCTGAAGATTTTGCAACTTATTACTGC |
| | | | CAACAGTCTAATAGTTACCCTCTCACTTTCGGCGGAGGGACCAA |
| | | | GGTCGAGATCAAA |
| 809 | YANG-1401c | VL domain | DIQMTQSPSSLSASVGDRVTITCRASQGISSWLAWYQQKPEKAP |
| | | amino | KSLIYSASTLQSGVPSRFSGSGSGTDFTLTISSLQPEDFATYYC |
| | | acid | QQSNSYPLTFGGGTKVEIK |
| | | sequence | |
| 810 | YANG-1401c | LCDR1 | QGISSW |
| 517 | YANG-1401c | LCDR2 | SAS |
| 811 | YANG-1401c | LCDR3 | QQSNSYPLT |
| 812 | YANG-1401d | VH domain | CAGGTGCAGCTGCAGGAGTCGGGCCCAGGACTGGTGAAGCCTTC |
| | | nucleic | GGGGACCCTGTCCCTCACCTGCGTTGTCTCTGGTGGCTCCTTCA |
| | | acid | GCAATACTAATTGGTGGAGTTGGGTCCGCCAGCCCCCAGGGAAG |
| | | sequence | GGGCTGGAGTGGATTGGGGAAGTCTTTCATAGTGGGAGCACCAA |
| | | | CTACAACCCGTCCCTCAAGAGTCGAGTCACCATATCAGTAGACA |
| | | | AGTCCAAGAACCAGTTCTCCCTGAACCTGAGTTCTGTGACCGCC |
| | | | GCGGACACGGCCGTGTATTACTGTGCGAGGGGGTATTATGGTGA |
| | | | CAATTGGTTCGACTCCTGGGGCCAGGGAACCCTGGTCACCGTCT |
| | | | CCTCA |
| 813 | YANG-1401d | VH domain | QVQLQESGPGLVKPSGTLSLTCVVSGGSFSNTNWWSWVRQPPGK |
| | | amino | GLEWIGEVFHSGSTNYNPSLKSRVTISVDKSKNQFSLNLSSVTA |
| | | acid | ADTAVYYCARGYYGDNWEDSWGQGTLVTVSS |
| | | sequence | |
| 814 | YANG-1401d | HCDR1 | GGSFSNTNW |
| 815 | YANG-1401d | HCDR2 | VFHSGST |
| 816 | YANG-1401d | HCDR3 | ARGYYGDNWEDS |
| 817 | YANG-1401d | VL domain | GACATCCAGATGACCCAGTCTCCATCCTCACTGTCTGCATCTGT |
| | | nucleic | AGGAGACAGAGTCACCATCACTTGTCGGGCGAGTCAGGGTATTA |
| | | acid | GCAGCTGGTTGGCCTGGTATCAGCAGAAACCAGAGAAAGCCCCT |
| | | sequence | AAGGCCCTGATCTATTCTGCATCCACTTTGCAAAGTGGAGTCCC |
| | | | ATCAAGGTTCAGCGGCAGTGGATCTGGGACAGATTTCACTCTCA |
| | | | CCATCAGCAGCCTGCAGCCTGAAGATGTTGCGACTTATTACTGC |
| | | | CAACAGTCTAGTAGTTACCCTCTCACTTTCGGCGGAGGGACCAA |
| | | | GGTCGAGATCAAA |
| 818 | YANG-1401d | VL domain | DIQMTQSPSSLSASVGDRVTITCRASQGISSWLAWYQQKPEKAP |
| | | amino | KALIYSASTLQSGVPSRFSGSGSGTDFTLTISSLQPEDVATYYC |
| | | acid | QQSSSYPLTFGGGTKVEIK |
| | | sequence | |
| 819 | YANG-1401d | LCDR1 | QGISSW |
| 517 | YANG-1401d | LCDR2 | SAS |
| 820 | YANG-1401d | LCDR3 | QQSSSYPLT |
| 821 | YANG-1401e | VH domain | CAGGTGCACCTGCAGGAGTCGGGCCCAGGACTGGTGAAGCCTTC |
| | | nucleic | GGGGACCCTGTCCCTCACCTGCGCTGTCTCTGGTGGCTCCTTCA |
| | | acid | GCCAGATTAACTGGTGGAGTTGGGTCCGCCAGTCCCCAGGAAAG |
| | | sequence | GGGCTGGAGTGGATTGGAGAAATCTATCATAGTGGGAGCACCAA |
| | | | CTACAACCCGTCCCTCAAGAGTCGAGTCACCATGTCCGTAGACA |
| | | | AGTCCAAGAACCACTTCTCCCTGGCGTTGAATTCTGTGACCGCC |
| | | | GCGGACACGGCCGTGTATTATTGTGCGCGGGGGTATTATGGTGA |
| | | | CAACTGGTTCGACTCATGGGGCCAGGGAACCCTGGTCACCGTCT |
| | | | CCTCA |
| 822 | YANG-1401e | VH domain | QVHLQESGPGLVKPSGTLSLTCAVSGGSFSQINWWSWVRQSPGK |
| | | amino | GLEWIGEIYHSGSTNYNPSLKSRVTMSVDKSKNHFSLALNSVTA |
| | | acid | ADTAVYYCARGYYGDNWEDSWGQGTLVTVSS |
| | | sequence | |
| 823 | YANG-1401e | HCDR1 | GGSFSQINW |
| 824 | YANG-1401e | HCDR2 | IYHSGST |
| 825 | YANG-1401e | HCDR3 | ARGYYGDNWEDS |
| 826 | YANG-1401e | VL domain | GACATCCAGATGACCCAGTCTCCATCCTCACTGTCTGCTTCTAT |
| | | nucleic | AGGCGACAGAGTCACCATCACTTGTCGGGCGAGTCAGGGTATTA |
| | | acid | GCAACTGGTTAGTCTGGTATCAGCAGAAACCAGAGAAAGCCCCT |
| | | sequence | AAGTCCCTGATCTATGCTGCTTCCAGTTTGCAAAGTGGGGTCCC |
| | | | ATCACGGTTCAGCGGCAGTGGATCTGGGACAGATTTCACTCTCA |
| | | | CCATCAGCAGCCTGCATCCTGAAGATTTTGCATCTTATTACTGC |
| | | | CAACAGTATAGTGATTACCCTCTCACTTTCGGCGGCGGGACCAG |
| | | | GGTGGAGATCAAA |
| 827 | YANG-1401e | VL domain | DIQMTQSPSSLSASIGDRVTITCRASQGISNWLVWYQQKPEKAP |
| | | amino | KSLIYAASSLQSGVPSRFSGSGSGTDFTLTISSLHPEDFASYYC |
| | | acid | QQYSDYPLTFGGGTRVEIK |
| | | sequence | |
| 828 | YANG-1401e | LCDR1 | QGISNW |
| 18 | YANG-1401e | LCDR2 | AAS |
| 829 | YANG-1401e | LCDR3 | QQYSDYPLT |
| 830 | YANG-1402 | VH domain | GAGGTGCAGCTGGTGGAGTCTGGAGGAGGCTTGATCCAGCCTGG |
| | | nucleic | GGGGTCCCTGAGACTCTCCTGTGCAGCCTCTGGGGTCACCGTCA |
| | | acid | GTAGTAACTACATGAACTGGGTCCGCCAGGCTCCAGGGAAGGGG |
| | | sequence | CTGGAGTGGGTCTCAGTTATTTATAGCGGTGGTAGCACATACTA |
| | | | CGCAGACTCCGTGAAGGGCCGATTCACCATCTCCAGAGACAATT |
| | | | CCAAGAACACGCTGTATCTTCAAATGAACAGCCTAAGAGTCGAG |
| | | | GACACGGCCGTATATTACTGTGCGAGAGACATAGGGGACTACGG |
| | | | TATGGACGTCTGGGGCCAAGGGACCACGGTCACCGTCTCCTCA |
| 831 | YANG-1402 | VH domain | EVQLVESGGGLIQPGGSLRLSCAASGVTVSSNYMNWVRQAPGKG |
| | | amino | LEWVSVIYSGGSTYYADSVKGRFTISRDNSKNTLYLQMNSLRVE |
| | | acid | DTAVYYCARDIGDYGMDVWGQGTTVTVSS |
| | | sequence | |
| 832 | YANG-1402 | HCDR1 | GVTVSSNY |
| 14 | YANG-1402 | HCDR2 | IYSGGST |
| 833 | YANG-1402 | HCDR3 | ARDIGDYGMDV |
| 834 | YANG-1402 | VL domain | GACATCCAGTTGACCCAGGCTCCATCCTTCCTGTCTGCATCTGT |
| | | nucleic | AGGAGACAGACTCACCATCACTTGCTGGGCCAGTCAGGGCATTA |
| | | acid | GCAGTTATTTAGCCTGGTATCAGCAAAAACCAGGGAAAGCCCCT |
| | | sequence | AAGATCCTGATCTATGCTGCATCCACTTTGCAAAGTGGGGTCCC |
| | | | ATCAAGGTTCAGCGGCAGTGGATCTGGGACAGAATTCACTCTCA |
| | | | CAATCAGCAGCCTGCAGCCTGAAGATTTTGCAACTTATTACTGT |
| | | | CAACAGCTTAATAGTTACCCGCTCATTTTCGGCGGAGGGACCAA |
| | | | GGTGGAGATCAAA |
| 835 | YANG-1402 | VL domain | DIQLTQAPSFLSASVGDRLTITCWASQGISSYLAWYQQKPGKAP |
| | | amino | KILIYAASTLQSGVPSRFSGSGSGTEFTLTISSLQPEDFATYYC |
| | | acid | QQLNSYPLIFGGGTKVEIK |
| | | sequence | |
| 8 | YANG-1402 | LCDR1 | QGISSY |
| 18 | YANG-1402 | LCDR2 | AAS |
| 836 | YANG-1402 | LCDR3 | QQLNSYPLI |
| 837 | YANG-1403 | VH domain | CAGGTGCAGCTGGTGGAGTCTGGGGGAGGCGTGGTCCAGCCTGG |
| | | nucleic | GAGGTCCCTGAGACTCTCCTGTGCAGCGTCTGGATTCACCTTCA |
| | | acid | GTAATTATGGCATGCACTGGGTCCGCCAGGCTCCAGGCAAGGGG |
| | | sequence | CTGGAGTGGGTGGCAGTTATATGGTATGATGGAAGTAATAAATA |
| | | | CTATGCAGACTCCGTGAAGGGCCGATTCACCATCTCCAGAGACA |
| | | | ATTCCAAGAACACGCTGTATCTGCAAATGAACAGCCTGAGAGCC |
| | | | GAGGACACGGCTGTTTATTTCTGTGTGAGAGAAACTGTTACGGA |
| | | | CGGTATGGACGTCTGGGGCCAAGGGACCACGGTCACCGTCTCCC |
| | | | TA |
| 838 | YANG-1403 | VH domain | QVQLVESGGGVVQPGRSLRLSCAASGFTFSNYGMHWVRQAPGKG |
| | | amino | LEWVAVIWYDGSNKYYADSVKGRFTISRDNSKNTLYLQMNSLRA |
| | | acid | EDTAVYFCVRETVTDGMDVWGQGTTVTVSL |
| | | sequence | |
| 839 | YANG-1403 | HCDR1 | GFTFSNYG |
| 840 | YANG-1403 | HCDR2 | IWYDGSNK |
| 841 | YANG-1403 | HCDR3 | VRETVTDGMDV |
| 842 | YANG-1403 | VL domain | AACATCCAGATGACCCAGTCTCCATCTGCCATGTCTGCATCTGT |
| | | nucleic | GGGAGACAGAGTCACCATCACTTGTCGGGCGAGGCAGGACATTA |
| | | acid | GCAATTATTTAGCCTGGTTTCAGCAGAAACCAGGGAAAGTCCCT |
| | | sequence | AAGCACCTGATCTATGCTGCATCCAGTTTGCTAAGTGGGGTCCC |
| | | | ATCAAGGTTCAGCGGCAGTGGATCTGGGACAGAATTCACTCTCA |
| | | | CAATCAGCAGCCTGCAGCCTGAAGATTTTGCAACTTATTACTGT |
| | | | CTACACCATAATGGTTACCCGTGGACGTTCGGCCAAGGGACCAA |
| | | | GGTGGAAATCAAA |
| 843 | YANG-1403 | VL domain | NIQMTQSPSAMSASVGDRVTITCRARQDISNYLAWFQQKPGKVP |
| | | amino | KHLIYAASSLLSGVPSRFSGSGSGTEFTLTISSLQPEDFATYYC |
| | | acid | LHHNGYPWTFGQGTKVEIK |
| | | sequence | |
| 844 | YANG-1403 | LCDR1 | QDISNY |
| 18 | YANG-1403 | LCDR2 | AAS |
| 845 | YANG-1403 | LCDR3 | LHHNGYPWT |
| 846 | YANG-2101 | VH domain | GAGGTGCAGCTGGTGGAGTCTGGGGGAGGTTTGGTACAGCCTGG |
| | | nucleic | GGGGTCCCTGAGACTCTCCTGTGCAGCCTCTGGATTCACCTTCA |
| | | acid | GTCTCTACGACATGCACTGGGTCCGTCAAGCAACAGGAAAAGGT |
| | | sequence | CTGGAGTGGGTCGCAGGTGTTGGTATTGCCGGTGACACCATCTA |
| | | | TCCAGACTCCGTGAAGGGCCGATTCACCATCTCCAGAGAGAATG |
| | | | CCAAGAACTCCTTCTTTCTTCAAATGAACAGACTGAGAGCCGGG |
| | | | GACACGGCTGTGTATTACTGTGTAAGAGGAGGAACTGGAACAAC |
| | | | TTTCTTTGATTACTGGGGCCAGGGAGTCCTGGTCACCGTCTCCT |
| | | | CT |
| 847 | YANG-2101 | VH domain | EVQLVESGGGLVQPGGSLRLSCAASGFTFSLYDMHWVRQATGKG |
| | | amino | LEWVAGVGIAGDTIYPDSVKGRFTISRENAKNSFFLQMNRLRAG |
| | | acid | DTAVYYCVRGGTGTTFFDYWGQGVLVTVSS |
| | | sequence | |
| 848 | YANG-2101 | HCDR1 | GFTFSLYD |
| 849 | YANG-2101 | HCDR2 | VGIAGDT |
| 850 | YANG-2101 | HCDR3 | VRGGTGTTFFDY |
| 851 | YANG-2101 | VL domain | GACATCCAGATGACCCAGTCTCCATCTTCACTGTCTGCATCTGT |
| | | nucleic | AGGAGACAGAGTCACCGTCACTTGTCGGGCGAGTCAGGACATTA |
| | | acid | CCAATTATTTAGCCTGGTTTCAGCAGAAACCAGGGAAAGCCCCT |
| | | sequence | AAGTCCCTGATCTATAGTGCATCCAGTTTGCAAGGTGGGACCCC |
| | | | CTCAAAGTTCAGCGGCAGTGGATCTGGGACAGAATTCACTCTCA |
| | | | CCATCAGCAGCCTCCAGCCTGAAGATTTTGCAACTTATTACTGC |
| | | | CAGCAGTATAATAGTTTCCCGCTCACTTTCGGCGGAGGGACCAA |
| | | | GGTGGAGATCAAA |
| 852 | YANG-2101 | VL domain | DIQMTQSPSSLSASVGDRVTVTCRASQDITNYLAWFQQKPGKAP |
| | | amino | KSLIYSASSLQGGTPSKFSGSGSGTEFTLTISSLQPEDFATYYC |
| | | acid | QQYNSFPLTFGGGTKVEIK |
| | | sequence | |
| 853 | YANG-2101 | LCDR1 | QDITNY |
| 517 | YANG-2101 | LCDR2 | SAS |
| 854 | YANG-2101 | LCDR3 | QQYNSFPLT |
| 855 | YANG-2102 | VH domain | GAGGTGAAGCTGGTGGAGTCTGGGGGAGGCTTGGTACAGCGGGG |
| | | nucleic | GGGGTCCCTGAGACTCTCCTGTGCAGCCTCTGGATTCACCTTCA |
| | | acid | GTAGCCACGCCATGTACTGGGTCCGTCAAATTCCAGGAAAAGGT |
| | | sequence | CTGGAGTGGGTCGCAGGTATTGGTGTTGCTGGCGACACATTTTA |
| | | | TCCAGACTCCGTGAAGGGCCGATTCACCATCTCCAGAGAAAATG |
| | | | CCAACAACTCCTTGTCTCTTCAAATGGACAGCCTGAGAACCGGG |
| | | | GACACGGCTATATATTACTGTGTCAGAGATGGTTATAGTGGGAG |
| | | | CTACCCTTACCACTACTACGGTATGGACGTCTGGGGCCAAGGGA |
| | | | TCACGGTCGTCGTCTCCTCA |
| 856 | YANG-2102 | VH domain | EVKLVESGGGLVQRGGSLRLSCAASGFTFSSHAMYWVRQIPGKG |
| | | amino | LEWVAGIGVAGDTFYPDSVKGRFTISRENANNSLSLQMDSLRTG |
| | | acid | DTAIYYCVRDGYSGSYPYHYYGMDVWGQGITVVVSS |
| | | sequence | |
| 857 | YANG-2102 | HCDR1 | GFTFSSHA |
| 858 | YANG-2102 | HCDR2 | IGVAGDT |
| 859 | YANG-2102 | HCDR3 | VRDGYSGSYPYHYYGMDV |
| 860 | YANG-2102 | VL domain | GACATCCAGTTGACCCAGTCTCCATCCTCACTGTCTGCATCTGT |
| | | nucleic | AGGAGACAGTGTCACCATCACTTGTCGGGCGAGTCAGGGCATTG |
| | | acid | ACACTTATTTAGCCTGGTTTCAGCAGAAACCAGGGAAAGCCCCT |
| | | sequence | AAGTCCCTGATCTATGTTGCATCCAGTTTACAGAGTGGGGTCCC |
| | | | ATCAAAATTCAGCGGCAGTGGATCTGGGACAGATTTCACTCTCA |
| | | | CCATCAGCAGCCTGCTGCCTGAAGATTTTGCAACTTATTACTGC |
| | | | CAACAGTATAAGAGTTACCCGTGGACGTTCGGCCAAGGGACCAA |
| | | | GGTGGAGATCAAA |
| 861 | YANG-2102 | VL domain | DIQLTQSPSSLSASVGDSVTITCRASQGIDTYLAWFQQKPGKAP |
| | | amino | KSLIYVASSLQSGVPSKFSGSGSGTDFTLTISSLLPEDFATYYC |
| | | acid | QQYKSYPWTFGQGTKVEIK |
| | | sequence | |
| 862 | YANG-2102 | LCDR1 | QGIDTY |
| 863 | YANG-2102 | LCDR2 | VAS |
| 864 | YANG-2102 | LCDR3 | QQYKSYPWT |
| 865 | YANG-2103 | VH domain | GAGGCGCAGTTGGTGGAGTCTGGAGGAGGCTTGATCCAGTCTGG |
| | | nucleic | GGGGTCCCTGAGACTCTCCTGTATAGCCTCTGGATTAACCGTCA |
| | | acid | ATAGCAACTACATGAACTGGGTCCGCCAGGCTCCAGGGAAGGGA |
| | | sequence | CTGGAGTGGGTCTCAGTTATTTATAGCGGTGGTACCACATTCTA |
| | | | CGCAGACTCCGTGAAGGGCCGATTCACCATCTCCAGAGACAATT |
| | | | CCAAGAACACGCTGTATCTTCAAATGACTAGTCTGAGAGCCGAG |
| | | | GACACGGCCGTGTATTATTGTGCGAGAGAGGGATACGGTATGGA |
| | | | CGTCTGGGGCCAAGGGACCACGGTCACCGTCTCCTCA |
| 866 | YANG-2103 | VH domain | EAQLVESGGGLIQSGGSLRLSCIASGLTVNSNYMNWVRQAPGKG |
| | | amino | LEWVSVIYSGGTTFYADSVKGRFTISRDNSKNTLYLQMTSLRAE |
| | | acid | DTAVYYCAREGYGMDVWGQGTTVTVSS |
| | | sequence | |
| 867 | YANG-2103 | HCDR1 | GLTVNSNY |
| 868 | YANG-2103 | HCDR2 | IYSGGTT |
| 514 | YANG-2103 | HCDR3 | AREGYGMDV |
| 869 | YANG-2103 | VL domain | TCCTATGTGCTGACTCAGCCACCCTCAGTGTCAGTGGCCCCAGG |
| | | nucleic | AAAGACGGCCAGGATAAACTGTGGGGGAAATAATTTTGGAAGTA |
| | | acid | AAAGTGTGCACTGGTACCAGCAGAAGCCAGGCCAGGCCCCTGTG |
| | | sequence | CTGGTCATCTATTATGATAGCGACCGGCCCTCAGGGATCCCTGA |
| | | | GCGATTCTCTGGCTCCAACTCAGGGAACACGGCCACCCTGACCA |
| | | | TCAGCAGGGTCGAAGCCGGGGATGAGGCCGACTATTACTGTCAG |
| | | | GTGTGGGATAATAGTAGTGATCATTTTGTCTTCGGAGCTGGGAC |
| | | | CAAGGTCACCGTCCTA |
| 870 | YANG-2103 | VL domain | SYVLTQPPSVSVAPGKTARINCGGNNFGSKSVHWYQQKPGQAPV |
| | | amino | LVIYYDSDRPSGIPERFSGSNSGNTATLTISRVEAGDEADYYCQ |
| | | acid | VWDNSSDHFVFGAGTKVTVL |
| | | sequence | |
| 871 | YANG-2103 | LCDR1 | NFGSKS |
| 568 | YANG-2103 | LCDR2 | YDS |
| 872 | YANG-2103 | LCDR3 | QVWDNSSDHFV |
| 873 | YANG-2104 | VH domain | GAAGTGAAGTTGGTGGAGTCTGGGGGAGGCTTGGTACAGCCTGG |
| | | nucleic | GGGGTCCCTGAGACTCTCCTGTGCAGCCTCTGGATTCACATTCA |
| | | acid | GTAGCCACGCCATGTACTGGGTCCGTCAAAGTCCAGGAAAAGGT |
| | | sequence | CTGGAGTGGGTCTCAGGTATTGGTGTTGCTGGTGACACATTTTA |
| | | | TGTAGACTCCGTGAAGGGCCGATTCACCATCTCCAGAGAAAATG |
| | | | CCAGGAACTCCTTGTCTCTTCAAATGAACAGCCTGAGAGCCGGG |
| | | | GACACGGCTGTTTATTACTGTGCCAGAGATGGTTATAGTGGGAC |
| | | | CCACCCTTACCACTTCTACGGTATGGACGTCTGGGGCCAAGGGA |
| | | | TCACGGTCGTCGTCTCCTCA |
| 874 | YANG-2104 | VH domain | EVKLVESGGGLVQPGGSLRLSCAASGFTFSSHAMYWVRQSPGKG |
| | | amino | LEWVSGIGVAGDTFYVDSVKGRFTISRENARNSLSLQMNSLRAG |
| | | acid | DTAVYYCARDGYSGTHPYHFYGMDVWGQGITVVVSS |
| | | sequence | |
| 875 | YANG-2104 | HCDR1 | GFTFSSHA |
| 876 | YANG-2104 | HCDR2 | IGVAGDT |
| 877 | YANG-2104 | HCDR3 | ARDGYSGTHPYHFYGMDV |
| 878 | YANG-2104 | VL domain | GACATCCAGATGACCCAGTCTCCATCCTCACTGTCTGCATCTGT |
| | | nucleic | AGGAGACAGTGTCACCATCACTTGTCGGGCGAGTCAGGACATTA |
| | | acid | ACACTTATTTAGCCTGGTTTCAGCAGAAACCAGGGAAAGCCCCT |
| | | sequence | AAGTCCCTGATCTATGTTGCATCCAGTTTACAGAGTGGGGTCCC |
| | | | ATCAAAGTTCAGCGGCAGTGGATCTGGGACAGATTTCACTCTCA |
| | | | CCATCAGCAGCCTGCTGCCTGAAGATTTTGCAACTTATTATTGC |
| | | | CAACAGTATAAAAGTTACCCGTGGACGTTCGGCCAAGGGACCAA |
| | | | GGTGGAGATCAAA |
| 879 | YANG-2104 | VL domain | DIQMTQSPSSLSASVGDSVTITCRASQDINTYLAWFQQKPGKAP |
| | | amino | KSLIYVASSLQSGVPSKFSGSGSGTDFTLTISSLLPEDFATYYC |
| | | acid | QQYKSYPWTFGQGTKVEIK |
| | | sequence | |
| 880 | YANG-2104 | LCDR1 | QDINTY |
| 863 | YANG-2104 | LCDR2 | VAS |
| 881 | YANG-2104 | LCDR3 | QQYKSYPWT |
| 882 | YANG-2105 | VH domain | GAGGTGCAACTGGTGGAGTCTGGAGGAGGCTTGATCCAGCCTGG |
| | | nucleic | GGGGTCCCTGAGACTCTCCTGTGCAGCCTCTGGGCTCACCGTCA |
| | | acid | GTAGCAATTACATGAACTGGGTCCGCCAGGCTCCAGGGAAGGGG |
| | | sequence | CTGGAGTGGGTCTCAGTTATTTATAGCGGTGGTAGTACATTCTA |
| | | | CACAGATTCCGTGAAGGGCCGATTCACCATCTCCAGAGACAACT |
| | | | CCAAGAACACGCTGAATCTTCAAATGAACAGTCTGCGAGCCGAG |
| | | | GACACGGCCGTGTATTATTGTGCGAGAGATCTGGGGATACGCGG |
| | | | GGGTATGGACGTCTGGGGCCAAGGGACCACGGTCACCGTCTCCT |
| | | | CA |
| 883 | YANG-2105 | VH domain | EVQLVESGGGLIQPGGSLRLSCAASGLTVSSNYMNWVRQAPGKG |
| | | amino | LEWVSVIYSGGSTFYTDSVKGRFTISRDNSKNTLNLQMNSLRAE |
| | | acid | DTAVYYCARDLGIRGGMDVWGQGTTVTVSS |
| | | sequence | |
| 172 | YANG-2105 | HCDR1 | GLTVSSNY |
| 14 | YANG-2105 | HCDR2 | IYSGGST |
| 884 | YANG-2105 | HCDR3 | ARDLGIRGGMDV |
| 885 | YANG-2105 | VL domain | GACATCCAGTTGATCCAGTCTCCATCCTTCCTGTCTGCATCTGT |
| | | nucleic | AGGAGACAGAGTCACCATCACTTGCTGGGCCAGTCAGGGCATTA |
| | | acid | GTAGTTATTTAGCCTGGTATCAGCAAAAACCAGGGAAAGCCCCT |
| | | sequence | AACCTCCTGATCTATGCTGCATCCACTTTGCAAAGTGGGGTCCC |
| | | | ATCAAGGTTCAGCGGCAGTGGATCTGGGACAGAGTTCACTCTCA |
| | | | CAATCAGCAGCCTGCAGCCTGAGGATTTTGCAACTTATTACTGT |
| | | | CAACAGCTTGATGGTTCCCTCACTTTCGGCGGAGGGACCAAGGT |
| | | | GGAGATCAAA |
| 886 | YANG-2105 | VL domain | DIQLIQSPSFLSASVGDRVTITCWASQGISSYLAWYQQKPGKAP |
| | | amino | NLLIYAASTLQSGVPSRFSGSGSGTEFTLTISSLQPEDFATYYC |
| | | acid | QQLDGSLTFGGGTKVEIK |
| | | sequence | |
| 8 | YANG-2105 | LCDR1 | QGISSY |
| 18 | YANG-2105 | LCDR2 | AAS |
| 887 | YANG-2105 | LCDR3 | QQLDGSLT |
| 888 | YANG-2106 | VH domain | CAGGTGCAGGTAGTGGAGTCTGGGGGAGGCGTGGTCCAGCCTGG |
| | | nucleic | GAGGTCCCTGAGACTCTCCTGTTCAGCGTCTGGATTCACCTTCA |
| | | acid | GCACCTATGGCATGCACTGGGTCCGCCAGGCTCCAGGCAAGGGG |
| | | sequence | CTGGAGTGGGTGGCAGTTATATGGTATGATGGAAGTAATAAGTA |
| | | | CTATGCAGACTCCGTGAAGGGCCGATTCATCATCTCCAGAGACA |
| | | | ATTCCAAGAACACGCTGTATCTGCAAATGAGCAGTCTGAGACCC |
| | | | GATGACACGGCTGTGTATTATTGTGTGAGAGAGGGAGTGGCTGA |
| | | | CGGTATGGGCGTCTGGGGCCAAGGGACCACAGTCACCGTCTCTT |
| | | | CA |
| 889 | YANG-2106 | VH domain | QVQVVESGGGVVQPGRSLRLSCSASGFTFSTYGMHWVRQAPGKG |
| | | amino | LEWVAVIWYDGSNKYYADSVKGRFIISRDNSKNTLYLQMSSLRP |
| | | acid | DDTAVYYCVREGVADGMGVWGQGTTVTVSS |
| | | sequence | |
| 890 | YANG-2106 | HCDR1 | GFTFSTYG |
| 891 | YANG-2106 | HCDR2 | IWYDGSNK |
| 892 | YANG-2106 | HCDR3 | VREGVADGMGV |
| 893 | YANG-2106 | VL domain | AACATCCAGATGACCCAGTCTCCATCTGCCATGTCTGCATCTGT |
| | | nucleic | GGGAGACAGAGTCACCATCACTTGTCGGGCGAGGCAGGACATTA |
| | | acid | GCACTTACTTAGCCTGGTTTCAGCAGAAACCAGGGAAAGTCCCT |
| | | sequence | AAGCACCTGATCTATGCTGCGTCTACTTTGCTAAGTGGGGTCCC |
| | | | ATCAAGGTTCGGCGGCAGTGGTTCTGGGACAGAATTCACTCTCA |
| | | | CAATCACCAGCCTGCAGCCTGAAGATTTTGCAACTTATTACTGT |
| | | | CTACACCATAATAATTATCCGTGGACGTTCGGCCAAGGGACCAA |
| | | | GGTGGAAATCAAA |
| 894 | YANG-2106 | VL domain | NIQMTQSPSAMSASVGDRVTITCRARQDISTYLAWFQQKPGKVP |
| | | amino | KHLIYAASTLLSGVPSRFGGSGSGTEFTLTITSLQPEDFATYYC |
| | | acid | LHHNNYPWTFGQGTKVEIK |
| | | sequence | |
| 895 | YANG-2106 | LCDR1 | QDISTY |
| 18 | YANG-2106 | LCDR2 | AAS |
| 896 | YANG-2106 | LCDR3 | LHHNNYPWT |
| 897 | YANG-2107 | VH domain | CAGGTGCAGCTGCAGGAGTCGGGCCCAGGACTGGTGAAGCCTTC |
| | | nucleic | GGGGACCCTGTCCCTCACCTGCGCTGTCTCTGGTGGCTCCATTA |
| | | acid | GAAGTAGTAACTGGTGGATTTGGGTCCGCCAGTCCCCAGGGAAG |
| | | sequence | GGGCTGGAGTGGATTGGGGAAATCTATCATGGTGGGAATACCAA |
| | | | CTATAACCCGTCCCTCAAGAGTCGAGTCACCTTGTCAGTAGACA |
| | | | AGTCCAAGAACCAGTTCTCCCTGAAGCTGAGTTCTGTGACCGCC |
| | | | GCGGACACGGCCGTATATTATTGTGCGAGAGCTCTTTACGATAT |
| | | | CGGGGGAGTTTTTGATATTTGGGGCCAGGGGACTATGGTCACCG |
| | | | TCTCTTCA |
| 898 | YANG-2107 | VH domain | QVQLQESGPGLVKPSGTLSLTCAVSGGSIRSSNWWIWVRQSPGK |
| | | amino | GLEWIGEIYHGGNTNYNPSLKSRVTLSVDKSKNQFSLKLSSVTA |
| | | acid | ADTAVYYCARALYDIGGVFDIWGQGTMVTVSS |
| | | sequence | |
| 899 | YANG-2107 | HCDR1 | GGSIRSSNW |
| 900 | YANG-2107 | HCDR2 | IYHGGNT |
| 901 | YANG-2107 | HCDR3 | ARALYDIGGVFDI |
| 902 | YANG-2107 | VL domain | CAGTCTGCCCTGACTCAGCCTGCCTCCGTGTCGGGGTCTCCTGG |
| | | nucleic | ACAGTCGATCTCCATCTCCTGCACTGGAACCAGCAGTGATATTG |
| | | acid | GTGGTTATAACTATGTCTCCTGGTACCAACAGCACCCAGGCAAA |
| | | sequence | GCCCCCAAACTCATGATTTATGATGTCAGTGAGCGGCCCTCAGG |
| | | | GGTTTCTAATCGCTTCTCTGGCTCCAAGTCTGGCAACACGGCCT |
| | | | CCCTGACAATCTCTGGGCTCCAGGCTGAGGACGAGGGTGATTAT |
| | | | TACTGCTGCTCATATGCAGCTAATAGTAATGTGGTATTCGGCGG |
| | | | AGGGACCAAACTGTCCGTCCTA |
| 903 | YANG-2107 | VL domain | QSALTQPASVSGSPGQSISISCTGTSSDIGGYNYVSWYQQHPGK |
| | | amino | APKLMIYDVSERPSGVSNRFSGSKSGNTASLTISGLQAEDEGDY |
| | | acid | YCCSYAANSNVVFGGGTKLSVL |
| | | sequence | |
| 904 | YANG-2107 | LCDR1 | SSDIGGYNY |
| 203 | YANG-2107 | LCDR2 | DVS |
| 905 | YANG-2107 | LCDR3 | CSYAANSNVV |
| 906 | YANG-2108 | VH domain | CAGGTGCAGCTGCAGGAGTCGGGCCCAGGACAGGTGAAGCCTTC |
| | | nucleic | GGGGACCCTGTCCCTCACCTGCGCTGTCTCTGGTGGCTCCATCA |
| | | acid | GAAGTAGTAACTGGTGGATTTGGGTCCGCCAGCCCCCAGGGAAG |
| | | sequence | GGTCTGGAGTGGATTGGGGAAATCTATCATGGTGGAAACACCAA |
| | | | CTACAGCCCGTCCCTCAAGAGTCGAGTCTCCATATCAGTAGACA |
| | | | AGTCCAAGAACCACTTCTCCCTGAACCTGACCTCTGTGACCGCC |
| | | | GCGGACACGGCCGTATATTACTGTGCGAGAGCTCTTTACGATGT |
| | | | CGGGGGAGTTTTTGATATTTGGGGCCAAGGGACTATGGTCACCG |
| | | | TCTCTTCA |
| 907 | YANG-2108 | VH domain | QVQLQESGPGQVKPSGTLSLTCAVSGGSIRSSNWWIWVRQPPGK |
| | | amino | GLEWIGEIYHGGNTNYSPSLKSRVSISVDKSKNHFSLNLTSVTA |
| | | acid | ADTAVYYCARALYDVGGVFDIWGQGTMVTVSS |
| | | sequence | |
| 908 | YANG-2108 | HCDR1 | GGSIRSSNW |
| 909 | YANG-2108 | HCDR2 | IYHGGNT |
| 910 | YANG-2108 | HCDR3 | ARALYDVGGVEDI |
| 911 | YANG-2108 | VL domain | CAGTCTGCCCTGACTCAGCCTGCCTCCGTGTCGGGGTCTCCTGG |
| | | nucleic | ACAGTCGATCACCATCTCCTGCACTGGAAGCAGCAGTGATGTTG |
| | | acid | GTGGTTATAATTATGTCTCCTGGTACCAACAGCACCCAGGCAAA |
| | | sequence | GCCCCCAAACTCATGATTTATGATGTCAGTGAGCGGCCCTCAGG |
| | | | GGTTTCTAATCGCTTCTCTGGCTCCAAGTCTGGCAACACGGCCT |
| | | | CCCTGACAATCTCTGGGCTCCAGGCTGAGGACGAGGGTGATTAT |
| | | | TACTGCTGCTCATATGCAGCTAATAGCAATGTGGTATTCGGCGG |
| | | | AGGGACCAAACTGTCCGTCCTA |
| 912 | YANG-2108 | VL domain | QSALTQPASVSGSPGQSITISCTGSSSDVGGYNYVSWYQQHPGK |
| | | amino | APKLMIYDVSERPSGVSNRFSGSKSGNTASLTISGLQAEDEGDY |
| | | acid | YCCSYAANSNVVFGGGTKLSVL |
| | | sequence | |
| 913 | YANG-2108 | LCDR1 | SSDVGGYNY |
| 203 | YANG-2108 | LCDR2 | DVS |
| 914 | YANG-2108 | LCDR3 | CSYAANSNVV |
| 915 | YANG-2108a | VH domain | CAGGTGCAGCTGCAGGAGTCGGGCCCAGGACAGGTGAAGCCTTC |
| | | nucleic | GGGGACCCTGTCCCTCACCTGCGCTGTCTCTGGAGGCTCCATCA |
| | | acid | GAAGTAGTAACTGGTGGATTTGGGTCCGCCAGTCCCCCGGGAAG |
| | | sequence | GGTCTGGAGTGGATTGGGGAAATCTATCATGGTGGGAACACCAA |
| | | | CTACAATCCGTCCCTCAAGAGTCGACTCACCATATCAATAGACA |
| | | | AGTCCAAGAACCACTTCTCCATGAAGCTGCACTCTGTGACCGCC |
| | | | GCGGACACGGCCGTATATTACTGTGCGAGAACTCTTTACGATAT |
| | | | CGGGGGAGTTTTTGATATTTGGGGCCAAGGGACTTTGGTCACCG |
| | | | TCTCTTCA |
| 916 | YANG-2108a | VH domain | QVQLQESGPGQVKPSGTLSLTCAVSGGSIRSSNWWIWVRQSPGK |
| | | amino | GLEWIGEIYHGGNTNYNPSLKSRLTISIDKSKNHFSMKLHSVTA |
| | | acid | ADTAVYYCARTLYDIGGVFDIWGQGTLVTVSS |
| | | sequence | |
| 917 | YANG-2108a | HCDR1 | GGSIRSSNW |
| 918 | YANG-2108a | HCDR2 | IYHGGNT |
| 919 | YANG-2108a | HCDR3 | ARTLYDIGGVFDI |
| 920 | YANG-2108a | VL domain | CAGTCTGCCCTGACTCAGCCTGCCTCCGTGTCGGGGTCTCCTGG |
| | | nucleic | ACAGTCGATCACCATCTCCTGCACTGGAACCAGCAGTGATGTTG |
| | | acid | GTGGTTATAACTATGTCTCCTGGTACCAACACCACCCAGGCAAA |
| | | sequence | GCCCCCAAACTCATGATTTATGATGTTAGTGAGCGGCCCTCAGG |
| | | | GGTTTCTAATCGCTTCTCTGGCTCCAAGTCTGGCAACACGGCCT |
| | | | CCCTGACAATCTCTGGGCTCCAGGCTGAGGACGAGGGTGATTAT |
| | | | TACTGCTGCTCATATGCAGCTAATAGCAATGTGGTATTCGGCGG |
| | | | AGGGACCAAACTGTCCGTCCTA |
| 921 | YANG-2108a | VL domain | QSALTQPASVSGSPGQSITISCTGTSSDVGGYNYVSWYQHHPGK |
| | | amino | APKLMIYDVSERPSGVSNRFSGSKSGNTASLTISGLQAEDEGDY |
| | | acid | YCCSYAANSNVVFGGGTKLSVL |
| | | sequence | |
| 922 | YANG-2108a | LCDR1 | SSDVGGYNY |
| 203 | YANG-2108a | LCDR2 | DVS |
| 923 | YANG-2108a | LCDR3 | CSYAANSNVV |
| 924 | YANG-2108b | VH domain | CAGGTGCAGCTGCAGGAGTCGGGCCCAGGACTGGTGAAGCCTTC |
| | | nucleic | GGGGACCCTGTCCCTCACCTGCGCTGTCTCTGGTGGTTCCATTA |
| | | acid | GAAGTAGTAACTGGTGGATTTGGGTCCGCCAGTCCCCAGGGAAG |
| | | sequence | GGGCTGGAGTGGATTGGGGAAATCTATCATGGTGGGAATACCAA |
| | | | CTATAACCCGTCCCTCAAGAGTCGAGTCACCATGTCAGTAGACA |
| | | | AGTCCAAGAACCAGTTCTCCCTGAAGCTGAGTTCTGTGACCGCC |
| | | | GCGGACACGGCCGTATATTACTGTGCGAGACCTCTTTACGATAT |
| | | | CGGGGGAGTTTTTGATATTTGGGGCCAGGGGACTATGGTCACCG |
| | | | TCTCTTCA |
| 925 | YANG-2108b | VH domain | QVQLQESGPGLVKPSGTLSLTCAVSGGSIRSSNWWIWVRQSPGK |
| | | amino | GLEWIGEIYHGGNTNYNPSLKSRVTMSVDKSKNQFSLKLSSVTA |
| | | acid | ADTAVYYCARPLYDIGGVFDIWGQGTMVTVSS |
| | | sequence | |
| 926 | YANG-2108b | HCDR1 | GGSIRSSNW |
| 927 | YANG-2108b | HCDR2 | IYHGGNT |
| 928 | YANG-2108b | HCDR3 | ARPLYDIGGVFDI |
| 929 | YANG-2108b | VL domain | CAGTCTGCCCTGACTCAGCCTGCCTCCGTGTCGGGGTCTCCTGG |
| | | nucleic | ACAGTCGATCTCCATCTCCTGCACTGGAACCAACAGTGATATTG |
| | | acid | GTGGTTATAACTATGTCTCCTGGTACCAACAGCACCCAGGCAAA |
| | | sequence | GCCCCCAAACTCATGATTTATGATGTCAGTGAGCGGCCCTCAGG |
| | | | GGTTTCTAATCGCTTCTCTGGCTCCAAGTCTGGCAACACGGCCT |
| | | | CCCTGACAATCTCTGGGCTCCAGGCTGAGGACGAGGGTGATTAT |
| | | | TACTGCTGCTCATATGCAAGTAATAGTAATGTGGTATTCGGCGG |
| | | | AGGGACCAAACTGTCCGTCCTA |
| 930 | YANG-2108b | VL domain | QSALTQPASVSGSPGQSISISCTGINSDIGGYNYVSWYQQHPGK |
| | | amino | APKLMIYDVSERPSGVSNRFSGSKSGNTASLTISGLQAEDEGDY |
| | | acid | YCCSYASNSNVVFGGGTKLSVL |
| | | sequence | |
| 931 | YANG-2108b | LCDR1 | NSDIGGYNY |
| 203 | YANG-2108b | LCDR2 | DVS |
| 932 | YANG-2108b | LCDR3 | CSYASNSNVV |
| 933 | YANG-2108c | VH domain | CAGGTGCAGCTGCAGGAGTCGGGCCCAGGACAGGTGAAGCCTTC |
| | | nucleic | GGGGACCCTGTCCCTCACCTGCGCTGTCTCTGGTGGCTCCATCA |
| | | acid | GAAGTAGTAACTGGTGGATTTGGGTCCGCCAGCCCCCAGGGAAG |
| | | sequence | GGTCTGGAGTGGATTGGGGAAATCTATCATGGTGGGAATACCAA |
| | | | CTACAAGCCGTCCCTCAAGAGTCGAGTCACCATATCAGTTGACA |
| | | | AGTCCAAGAACCAGTTCTCCCTCCAGTTGACTTCTGTGACCGCC |
| | | | GCGGACACGGCCGTATACTACTGTGCGAGAGCTCTTTATGATAT |
| | | | CGGGGGAGTTTTTGATATTTGGGGCCAAGGGACGATGGTCACCG |
| | | | TCTCTTCA |
| 934 | YANG-2108c | VH domain | QVQLQESGPGQVKPSGTLSLTCAVSGGSIRSSNWWIWVRQPPGK |
| | | amino | GLEWIGEIYHGGNTNYKPSLKSRVTISVDKSKNQFSLQLTSVTA |
| | | acid | ADTAVYYCARALYDIGGVFDIWGQGTMVTVSS |
| | | sequence | |
| 935 | YANG-2108c | HCDR1 | GGSIRSSNW |
| 936 | YANG-2108c | HCDR2 | IYHGGNT |
| 937 | YANG-2108c | HCDR3 | ARALYDIGGVEDI |
| 938 | YANG-2108c | VL domain | CAGTCTGCCCTGACTCAGCCTGCCTCCGTGTCGGGGTCTCCTGG |
| | | nucleic | ACAGTCGATCACCATCTCCTGCACTGGAACCAGCAGTGATGTTG |
| | | acid | GTGGTTATAACTATGTCTCCTGGTACCAACAGTATCCAGGCAAA |
| | | sequence | GCCCCCAAACTCATGATTTATGATGTCAGTGAGCGGCCCTCAGG |
| | | | GGTTTCTAATCGCTTCTCTGGCTCCAAGTCTGGCAACACGGCCT |
| | | | CCCTGACAATCTCTGGGCTCCAGGCTGAGGACGAGGGTGATTAT |
| | | | TACTGCTGCTCATATGCAACTAATAGCAATGTGGTATTCGGCGG |
| | | | AGGGACCAAACTGTCCGTCCTA |
| 939 | YANG-2108c | VL domain | QSALTQPASVSGSPGQSITISCTGTSSDVGGYNYVSWYQQYPGK |
| | | amino | APKLMIYDVSERPSGVSNRFSGSKSGNTASLTISGLQAEDEGDY |
| | | acid | YCCSYATNSNVVFGGGTKLSVL |
| | | sequence | |
| 940 | YANG-2108c | LCDR1 | SSDVGGYNY |
| 203 | YANG-2108c | LCDR2 | DVS |
| 941 | YANG-2108c | LCDR3 | CSYATNSNVV |
| 942 | YANG-2108d | VH domain | CAGGTGCAGCTGCAGGAGTCGGGCCCAGGACTGGTGAAGCCTTC |
| | | nucleic | GGGGACCCTGTCCCTCACCTGCGCTGTCTCTGGTGGCTCCATTA |
| | | acid | GAAGTAGTAACTGGTGGATTTGGGTCCGCCAGTCCCCAGGGAAG |
| | | sequence | GGACTGGAGTGGATTGGGGAAATCTATCATGGTGGGAATACCAA |
| | | | GTATAATCCGTCCCTCAAGAGTCGAGTCACCATGTCAGTAGACA |
| | | | AGTCCAAGAACCAGTTCTCCCTGAAGCTGAGTTCTGTGACCGCC |
| | | | GCGGACACGGCCGTATATTACTGTGCGAGAGCTCTTTACGATAT |
| | | | CGGGGGAGTTTTTGATATTTGGGGCCAGGGGACTATGGTCACCG |
| | | | TCTCTTCA |
| 943 | YANG-2108d | VH domain | QVQLQESGPGLVKPSGTLSLTCAVSGGSIRSSNWWIWVRQSPGK |
| | | amino | GLEWIGEIYHGGNTKYNPSLKSRVTMSVDKSKNQFSLKLSSVTA |
| | | acid | ADTAVYYCARALYDIGGVFDIWGQGTMVTVSS |
| | | sequence | |
| 944 | YANG-2108d | HCDR1 | GGSIRSSNW |
| 945 | YANG-2108d | HCDR2 | IYHGGNT |
| 946 | YANG-2108d | HCDR3 | ARALYDIGGVFDI |
| 947 | YANG-2108d | VL domain | CAGTCTGCCCTGACTCAGCCTGCCTCCGTGTCGGGGTCTCCTGG |
| | | nucleic | ACAGTCGATCTCCATCTCCTGCACTGGAACCAGCAGTAATATTG |
| | | acid | GTGGTTATAACTATGTCTCCTGGTACCAACAGCACCCAGGCAAA |
| | | sequence | GCCCCCAAACTCATGATTTATGATGTCAGTGAGCGGCCCTCAGG |
| | | | GGTTTCTAATCGCTTCTCTGGCTCCAAGTCTGGCAACACGGCCT |
| | | | CCCTGACAATCTCTGGGCTCCAGGCTGAGGACGAGGGTGATTAT |
| | | | TACTGCTGCTCATATGGAAGTAATAGTAATGTGGTTTTCGGCGG |
| | | | AGGGACCAAACTGTCCGTCCTA |
| 948 | YANG-2108d | VL domain | QSALTQPASVSGSPGQSISISCTGTSSNIGGYNYVSWYQQHPGK |
| | | amino | APKLMIYDVSERPSGVSNRFSGSKSGNTASLTISGLQAEDEGDY |
| | | acid | YCCSYGSNSNVVFGGGTKLSVL |
| | | sequence | |
| 949 | YANG-2108d | LCDR1 | SSNIGGYNY |
| 203 | YANG-2108d | LCDR2 | DVS |
| 950 | YANG-2108d | LCDR3 | CSYGSNSNVV |
| 951 | YANG-2108e | VH domain | CAGGTGCAGCTGCAGGAGTCGGGCCCAGGACAGGTGAAGCCTTC |
| | | nucleic | GGGGACCCTGTCCCTCACCTGCGCTGTCTCTGGTGGCTCCATCA |
| | | acid | GAAGTAGTAACTGGTGGATTTGGGTCCGCCAGTCCCCCGGGAAG |
| | | sequence | GGTCTGGAGTGGATTGGGGAAATCTATCATGGTGGGAACACCAA |
| | | | CTACAACCCGTCCCTCAAGAGTCGACTCACCATATCAGTAGACA |
| | | | AGTCCAAGAACCAGTTCTCCATGAAGCTGAGCTCTGTGACCGCC |
| | | | GCGGACACGGCCGTATATTACTGTGCGAGAGCTCTTTACGATAT |
| | | | CGGGGGAGTTTTTGATATTTGGGGCCAAGGGACTATGGTCACCG |
| | | | TCTCTTCA |
| 952 | YANG-2108e | VH domain | QVQLQESGPGQVKPSGTLSLTCAVSGGSIRSSNWWIWVRQSPGK |
| | | amino | GLEWIGEIYHGGNTNYNPSLKSRLTISVDKSKNQFSMKLSSVTA |
| | | acid | ADTAVYYCARALYDIGGVFDIWGQGTMVTVSS |
| | | sequence | |
| 953 | YANG-2108e | HCDR1 | GGSIRSSNW |
| 954 | YANG-2108e | HCDR2 | IYHGGNT |
| 955 | YANG-2108e | HCDR3 | ARALYDIGGVFDI |
| 956 | YANG-2108? | VL domain | CAGTCTGCCCTGACTCAGCCTGCCTCCGTGTCGGGGTCTCCTGG |
| | | nucleic | ACAGTCGATCACCATCTCCTGCACTGGAACCAGCAGTGATGTTG |
| | | acid | GTGGTTATAACTATGTCTCCTGGTACCAACAGCACACAGGCAAA |
| | | sequence | GCCCCCAAATTCATGATTTATGATGTTAGTGAGCGGCCCTCAGG |
| | | | GGTTTCTAATCGCTTCTCTGGCTCCAAGTCTGGCAACACGGCCT |
| | | | CCCTGACAATCTCTGGGCTCCAGGCTGAGGACGAGGGTGATTAT |
| | | | TACTGCTGCTCATATGCAGCTGATAGCAATGTGGTTTTCGGCGG |
| | | | AGGGACCAAACTGTCCGTCCTA |
| 957 | YANG-2108? | VL domain | QSALTQPASVSGSPGQSITISCTGTSSDVGGYNYVSWYQQHTGK |
| | | amino | APKFMIYDVSERPSGVSNRFSGSKSGNTASLTISGLQAEDEGDY |
| | | acid | YCCSYAADSNVVFGGGTKLSVL |
| | | sequence | |
| 958 | YANG-2108e | LCDR1 | SSDVGGYNY |
| 203 | YANG-2108e | LCDR2 | DVS |
| 959 | YANG-2108e | LCDR3 | CSYAADSNVV |
| 960 | YANG-2108f | VH domain | CAAATGCAGCTGCAGGAGTCGGGCCCAGGACTGGTGAAGCCTTC |
| | | nucleic | GGGGACCCTGTCCCTCACCTGCGCTGTCTCTGGTGGCTCCATCA |
| | | acid | GAAGTAGTAACTGGTGGATTTGGGTCCGCCAGCCCCCAGGGAAG |
| | | sequence | GGACTGGAGTGGATTGGGGAAATCTCTCACGGTGGGAACACCAA |
| | | | CTACAACCCGTCCCTCAAAAGTCGAGTCACCATAACTGTAGACA |
| | | | AGTCCAAGAACCAGTTCTCCCTGAAACTGCCCTCTATGACCGCC |
| | | | GCGGACACGGCCATATATTACTGTGCGAGAGCTCTTTACGATAT |
| | | | CGGGGGAGTCTTTGATATTTGGGGCCCAGGGACTATGGTCACCG |
| | | | TCTCTTCA |
| 961 | YANG-2108f | VH domain | QMQLQESGPGLVKPSGTLSLTCAVSGGSIRSSNWWIWVRQPPGK |
| | | amino | GLEWIGEISHGGNTNYNPSLKSRVTITVDKSKNQFSLKLPSMTA |
| | | acid | ADTAIYYCARALYDIGGVFDIWGPGTMVTVSS |
| | | sequence | |
| 962 | YANG-2108f | HCDR1 | GGSIRSSNW |
| 963 | YANG-2108f | HCDR2 | ISHGGNT |
| 964 | YANG-2108f | HCDR3 | ARALYDIGGVEDI |
| 965 | YANG-2108f | VL domain | CAGTCTGCCCTGACTCAGCCTGCCTCCGTGTCGGGGTCTCCTGG |
| | | nucleic | ACAGTCGATCACCATGTCCTGCACTGGAACCAACAGTGATGTTG |
| | | acid | GTGGTTACAACTATGTCTCCTGGTACCAACAGCACCCAGGCAAA |
| | | sequence | GCCCCCAAACTCATGATTTATGATGTCAGTGAGCGGCCCTCAGG |
| | | | ACTTTCTAATCGCTTCTCTGGCTCCAAGTCTGGCAATACGGCCT |
| | | | CCCTGACAATCTCTGGGCTCCAGGCTGAGGACGAGGGTGATTAT |
| | | | TACTGCTGCTCATTTGCAGCTAATAGCAATGTGGTTTTCGGCGG |
| | | | GGGGACCAAGTTGTCCGTCCTA |
| 966 | YANG-2108f | VL domain | QSALTQPASVSGSPGQSITMSCTGTNSDVGGYNYVSWYQQHPGK |
| | | amino | APKLMIYDVSERPSGLSNRFSGSKSGNTASLTISGLQAEDEGDY |
| | | acid | YCCSFAANSNVVFGGGTKLSVL |
| | | sequence | |
| 967 | YANG-2108f | LCDR1 | NSDVGGYNY |
| 203 | YANG-2108f | LCDR2 | DVS |
| 968 | YANG-2108f | LCDR3 | CSFAANSNVV |
| 2369 | YANG-2108g | VH domain | CAGGTGCAGCTGCAGCAGTCGGGCCCAGGACTGCTGAAGCCTTC |
| | | nucleic | GGGGACCCTGTCCCTCACCTGCGCTGTCTCTGGTGGCTCCATCA |
| | | acid | GAAGTAGTAACTGGTGGATTTGGGTCCGCCAGTCCCCAGGGAAG |
| | | sequence | GGGCTGGAGTGGATTGGGGAAATCTATCATGGTGGGAACACCAA |
| | | | TTACAACCCGTCCCTCAAGAGTCGAGTCACCATATCAGTCGACA |
| | | | AGTCCAAGCACCAGTTCTCCCTGAAGCTGACCTCTGTGACCGCC |
| | | | GCGGACACGGCCGTCTATTACTGTGCGAGAGCTCTTTACGATAT |
| | | | CGGGGGAGTTTTTGATCTTTGGGGCCAAGGGACTATGGTCACCG |
| | | | TCTCTTCA |
| 2370 | YANG-2108g | VH domain | QVQLQQSGPGLLKPSGTLSLTCAVSGGSIRSSNWWIWVRQSPGK |
| | | amino | GLEWIGEIYHGGNTNYNPSLKSRVTISVDKSKHQFSLKLTSVTA |
| | | acid | ADTAVYYCARALYDIGGVEDLWGQGTMVTVSS |
| | | sequence | |
| 969 | YANG-2108g | HCDR1 | GGSIRSSNW |
| 970 | YANG-2108g | HCDR2 | IYHGGNT |
| 971 | YANG-2108g | HCDR3 | ARALYDIGGVEDL |
| 972 | YANG-2108g | VL domain | CAGTCTGCCCTGACTCAGCCTGCCTCCGTGTCGGGGTCTCCTGG |
| | | nucleic | ACAGTCGATCACCATCTCCTGCACTGGAACCAGCAGTGATGTTG |
| | | acid | GTGGTTATAACTATGTCTCCTGGTACCAACAACACCCAGGCAAA |
| | | sequence | GCCCCCAAACTCATGATTTATGATGTCAGTGAGCGGCCCTCAGG |
| | | | GGTTTCTAATCGCTTCTCTGGCTCCAAGTCTGGCAACACGGCCT |
| | | | CCCTGACAATCTCTGGGCTCCAGGCTGAGGACGAGGGTGATTAT |
| | | | TACTGCTGCTCATATGCAGCTAATAGCAGTGTTGTATTCGGCGG |
| | | | AGGGACCAAACTGTCCGTCCTA |
| 973 | YANG-2108g | VL domain | QSALTQPASVSGSPGQSITISCTGTSSDVGGYNYVSWYQQHPGK |
| | | amino | APKLMIYDVSERPSGVSNRFSGSKSGNTASLTISGLQAEDEGDY |
| | | acid | YCCSYAANSSVVFGGGTKLSVL |
| | | sequence | |
| 974 | YANG-2108g | LCDR1 | SSDVGGYNY |
| 203 | YANG-2108g | LCDR2 | DVS |
| 975 | YANG-2108g | LCDR3 | CSYAANSSVV |
| 976 | YANG-2108h | VH domain | CAGGTACAGCTGCAGGAGTCGGGCCCAGGACTGGTGAAGCCTTC |
| | | nucleic | GGGGACCCTGTCCCTCACCTGTGCTGTCTCTGGTGGCTCCATCA |
| | | acid | GAAGTAGTAACTGGTGGATTTGGGTCCGCCAGCCCCCAGGGAAG |
| | | sequence | GGGCTGGAGTGGATTGGGGAAATCTATCATGGTGGGAACACCAA |
| | | | CTACAACCCGTCCCTCCAGAGTCGAGTCACCATATCAGTAGACA |
| | | | AGTCCAAGAACCAGTTCTCCCTGAATCTGAGTTCTGTGACCGCC |
| | | | GCGGACACGGCCGTATATTACTGTGCGAGAGCTCTTTACGATAT |
| | | | CGGGGGAGTTTTTGATATTTGGGGCCAAGGGACTTTGGTCACCG |
| | | | TCTCTTCA |
| 977 | YANG-2108h | VH domain | QVQLQESGPGLVKPSGTLSLTCAVSGGSIRSSNWWIWVRQPPGK |
| | | amino | GLEWIGEIYHGGNTNYNPSLQSRVTISVDKSKNQFSLNLSSVTA |
| | | acid | ADTAVYYCARALYDIGGVFDIWGQGTLVTVSS |
| | | sequence | |
| 978 | YANG-2108h | HCDR1 | GGSIRSSNW |
| 979 | YANG-2108h | HCDR2 | IYHGGNT |
| 980 | YANG-2108h | HCDR3 | ARALYDIGGVFDI |
| 981 | YANG-2108h | VL domain | CAGTCTGCCCTGACTCAGCCTGCCTCCGTGTCGGGGTCTCCTGG |
| | | nucleic | ACAGTCGACCACCATCTCCTGCACTGGAACCAGCAGTGATGTTG |
| | | acid | GTGGTTATAACTATGTCTCCTGGTACCAACAACACCCAGGCAAA |
| | | sequence | GCCCCCAAACTCATGATTTATGATGTCAGTGAGCGGCCCTCAGG |
| | | | GGTTTCTAATCGCTTCTCTGGCTCCAAGTCTGGCAACACGGCCT |
| | | | CCCTGACAATCTCTGGGCTCCAGGCTGAGGACGAGGGTGATTAT |
| | | | TACTGCTGCTCATATGCAGCTAATAGCAATGTGCTATTCGGCGG |
| | | | AGGGACCAAACTGTCCGTCCTA |
| 982 | YANG-2108h | VL domain | QSALTQPASVSGSPGQSTTISCTGTSSDVGGYNYVSWYQQHPGK |
| | | amino | APKLMIYDVSERPSGVSNRFSGSKSGNTASLTISGLQAEDEGDY |
| | | acid | YCCSYAANSNVLFGGGTKLSVL |
| | | sequence | |
| 983 | YANG-2108h | LCDR1 | SSDVGGYNY |
| 203 | YANG-2108h | LCDR2 | DVS |
| 984 | YANG-2108h | LCDR3 | CSYAANSNVL |
| 985 | YANG-2108i | VH domain | CAGGTGCAACTGCAGGAGTCGGGCCCAGGACTGGTGAAGCCTTC |
| | | nucleic | GGGGACCCTGTCCCTCACCTGCGCTGTCTCTGGTGGCTCCATCA |
| | | acid | GAAGTAGTAACTGGTGGATTTGGGTCCGCCAGTCCCCAGGGAAG |
| | | sequence | GGGCTGGAGTGGATTGGGGAAATCTATCATGGTGGGAACACCAA |
| | | | CTACAACCCGTCCCTCAAGAGTCGAGTCACCACATTAGTAGACA |
| | | | AGTCCAAAAACCAATTCTCCCTGAAATTGAGTTCTGTGACCGCC |
| | | | GCGGACACGGCCGTATATTACTGTGCGAGAGCTCTTTACGATAT |
| | | | CGGGGGAGTTTTTGATATTTGGAGCCAAGGGACGATGGTCACCG |
| | | | TCTCTTCA |
| 986 | YANG-2108i | VH domain | QVQLQESGPGLVKPSGTLSLTCAVSGGSIRSSNWWIWVRQSPGK |
| | | amino | GLEWIGEIYHGGNTNYNPSLKSRVTTLVDKSKNQFSLKLSSVTA |
| | | acid | ADTAVYYCARALYDIGGVFDIWSQGTMVTVSS |
| | | sequence | |
| 987 | YANG-2108i | HCDR1 | GGSIRSSNW |
| 988 | YANG-21081 | HCDR2 | IYHGGNT |
| 989 | YANG-2108i | HCDR3 | ARALYDIGGVEDI |
| 990 | YANG-2108i | VL domain | CAGTCTGCCCTGACTCAGCCTGCCTCCGTGTCGGGGTCTCCTGG |
| | | nucleic | ACAGTCGATCACCATCTCCTGCACTGGAACCATCAGTGATGTTG |
| | | acid | GTGGTTATAACTATGTCTCTTGGTACCAACAGCACCCAGGCAAA |
| | | sequence | GCCCCCAAACTCATGATTTATGATGTCAGTGAGCGGCCCTCAGG |
| | | | GATTTCTAATCGCTTCTCTGGCTCCAAGTCTGGCAACACGGCCT |
| | | | CCCTGACAATCTCTGGGCTCCAGACTGAGGACGAGGGTGATTAT |
| | | | TACTGCTGCTCATATGCAGCTAATAGCAATGTGGTATTCGGCGG |
| | | | AGGGACCAAACTGTCCGTCCTA |
| 991 | YANG-2108i | VL domain | QSALTQPASVSGSPGQSITISCTGTISDVGGYNYVSWYQQHPGK |
| | | amino | APKLMIYDVSERPSGISNRFSGSKSGNTASLTISGLQTEDEGDY |
| | | acid | YCCSYAANSNVVFGGGTKLSVL |
| | | sequence | |
| 992 | YANG-2108i | LCDR1 | ISDVGGYNY |
| 203 | YANG-2108i | LCDR2 | DVS |
| 993 | YANG-21081 | LCDR3 | CSYAANSNVV |
| 994 | YANG-2108j | VH domain | CAGGTGCAGCTGCAGGAGTCGGGCCCAGGACAGGTGAAGCCTTC |
| | | nucleic | GGGGACCCTGTCCCTCACCTGCGCTGTCTCTGGTGGCTCCATCA |
| | | acid | GAAGTAGTAACTGGTGGATTTGGGTCCGCCAGTCCCCAGGGAAG |
| | | sequence | GGTCTGGAGTGGATTGGGGAAATCTATCATGGTGGAAACACCAA |
| | | | CTACAACCCGTCCCTCAAGAGTCGAGTCACCATATCATTAGACA |
| | | | AGTCCAAGAACCAGTTCTCCCTGAACCTGACCTCTGTGACCGCC |
| | | | GCGGACACGGCCGTATATTACTGTGCGAGAGCTCTTTACGATAT |
| | | | CGGGGGAGTTTTTGATATTTGGGGCCAAGGGACTATGGTCATCG |
| | | | TCTCTTCA |
| 995 | YANG-2108j | VH domain | QVQLQESGPGQVKPSGTLSLTCAVSGGSIRSSNWWIWVRQSPGK |
| | | amino | GLEWIGEIYHGGNTNYNPSLKSRVTISLDKSKNQFSLNLTSVTA |
| | | acid | ADTAVYYCARALYDIGGVFDIWGQGTMVIVSS |
| | | sequence | |
| 996 | YANG-2108j | HCDR1 | GGSIRSSNW |
| 997 | YANG-2108j | HCDR2 | IYHGGNT |
| 998 | YANG-2108j | HCDR3 | ARALYDIGGVFDI |
| 999 | YANG-2108j | VL domain | CAGTCTGCCCTGACTCAGCCTGCCTCCGTGTCGGGGTCTCCTGG |
| | | nucleic | ACAGTCGATCACCATCTCCTGCACTGGAAGCAGCAGTGATGTTG |
| | | acid | GTGGTTATAACTATGTCTCCTGGTACCAACAGCACCCAGGCAAA |
| | | sequence | GCCCCCAAACTCATGATTTATGATGTCAGTGAGCGGCCCTCAGG |
| | | | GGTTTCTAGTCGATTTTCTGGCTCCAAGTCTGGCAGCACGGCCT |
| | | | CCCTGACAATCTCTGGGCTCCAGGCTGAGGACGAGGGTGATTAT |
| | | | TACTGCTGCTCATATGCAATTAATAGCGATGTGGTTTTCGGCGG |
| | | | AGGGACCAAACTGTCCGTCCTA |
| 1000 | YANG-2108j | VL domain | QSALTQPASVSGSPGQSITISCTGSSSDVGGYNYVSWYQQHPGK |
| | | amino | APKLMIYDVSERPSGVSSRFSGSKSGSTASLTISGLQAEDEGDY |
| | | acid | YCCSYAINSDVVFGGGTKLSVL |
| | | sequence | |
| 1001 | YANG-2108j | LCDR1 | SSDVGGYNY |
| 1002 | YANG-2108j | LCDR2 | DVS |
| 1003 | YANG-2108j | LCDR3 | CSYAINSDVV |
| 1004 | YANG-2108k | VH domain | CAGGTGCAGCTGCAGGAGTCGGGCCCAGGACAGGTGAAGCCTTC |
| | | nucleic | GGGGACCCTGTCCCTCACCTGCGCTGTCTCTGGTGGCTCCATCA |
| | | acid | GAAGTAGTAACTGGTGGATTTGGGTCCGCCAGTCCCCCGGGAAG |
| | | sequence | GGTCTGGAGTGGATTGGGGAAATCTATCATGGTGGGAAGACCAA |
| | | | CTACAACCCGTCCCTCAAGAGTCGACTCACCATATCAGTAGACA |
| | | | AGTCCAAGAACCAGTTCTCCATGAAGCTGAGCTCTGTGACCGCC |
| | | | GCGGACACGGCCGTATATCACTGTGCGAGAACTCTTTATGATAT |
| | | | CGGGGGAGTTTTTGATATTTGGGGCCAAGGGACTATGGTCACCG |
| | | | TCTCTTCA |
| 1005 | YANG-2108k | VH domain | QVQLQESGPGQVKPSGTLSLTCAVSGGSIRSSNWWIWVRQSPGK |
| | | amino | GLEWIGEIYHGGKTNYNPSLKSRLTISVDKSKNQFSMKLSSVTA |
| | | acid | ADTAVYHCARTLYDIGGVFDIWGQGTMVTVSS |
| | | sequence | |
| 1006 | YANG-2108k | HCDR1 | GGSIRSSNW |
| 1007 | YANG-2108k | HCDR2 | IYHGGKT |
| 1008 | YANG-2108k | HCDR3 | ARTLYDIGGVFDI |
| 1009 | YANG-2108k | VL domain | CAGTCTGCCCTGACTCAGCCTGCCTCCGTGTCGGGGTCTCCTGG |
| | | nucleic | ACAGTCGATCACCATCTCCTGCACTGGAACCAGCAGTGATGTTG |
| | | acid | GTGGTTATAACTATGTCTCCTGGTACCAACACCACACAGGCAAA |
| | | sequence | GCCCCCAAACTCATGATTTATGATGTTAGTGAGCGGCCCTCAGG |
| | | | GGTTTCTAATCGCTTCTCTGGCTCCAAGTCTGGCAACACGGCCT |
| | | | CCCTGACAATCTCTGGGCTCCAGGCTGAGGACGAGGGTGATTAT |
| | | | TACTGCTGCTCATATGCAGCTGATAGCAATGTGGTTTTCGGCGG |
| | | | AGGGACCAAACTGTCCGTCCTA |
| 1010 | YANG-2108k | VL domain | QSALTQPASVSGSPGQSITISCTGTSSDVGGYNYVSWYQHHTGK |
| | | amino | APKLMIYDVSERPSGVSNRFSGSKSGNTASLTISGLQAEDEGDY |
| | | acid | YCCSYAADSNVVFGGGTKLSVL |
| | | sequence | |
| 1011 | YANG-2108k | LCDR1 | SSDVGGYNY |
| 1012 | YANG-2108k | LCDR2 | DVS |
| 1013 | YANG-2108k | LCDR3 | CSYAADSNVV |
| 1014 | YANG-21081 | VH domain | CAGGTGCAGCTGCAGGAGTCGGGCCCAGGACAGGTGAAGCCTTC |
| | | nucleic | GGGGACCCTGTCCCTCACCTGCGCTGTCTCTGGTGGCTCCATCA |
| | | acid | GAAGTAGTAACTGGTGGATTTGGGTCCGCCAGCCCCCAGGGAAG |
| | | sequence | GGTCTGGAGTGGATTGGGGAAATCTATCATGGTGGAAATACCAA |
| | | | CTACAAGCCGTCCCTCAAGAGTCGAGTCACCATATCAGTTGACA |
| | | | TGTCCAAGAACCAGTTCTCCCTGCAGTTGACTTCTGTGACCGCC |
| | | | GCGGACACGGCCGTATACTACTGTGCGAGAGCTCTTTATGATAT |
| | | | CGGGGGAGTCTTTGATATTTGGGGCCAAGGGACGATGGTCACCG |
| | | | TCTCTTCA |
| 1015 | YANG-21081 | VH domain | QVQLQESGPGQVKPSGTLSLTCAVSGGSIRSSNWWIWVRQPPGK |
| | | amino | GLEWIGEIYHGGNTNYKPSLKSRVTISVDMSKNQFSLQLTSVTA |
| | | acid | ADTAVYYCARALYDIGGVFDIWGQGTMVTVSS |
| | | sequence | |
| 1016 | YANG-21081 | HCDR1 | GGSIRSSNW |
| 1017 | YANG-21081 | HCDR2 | IYHGGNT |
| 1018 | YANG-21081 | HCDR3 | ARALYDIGGVFDI |
| 1019 | YANG-21081 | VL domain | CAGTCTGCCCTGACTCAGCCTGCCTCCGTGTCGGGGTCTCCTGG |
| | | nucleic | ACAGTCGATCACCATCTCCTGCACTGGAACCAGCAGTGATGTTG |
| | | acid | GTGGTTATAACTATGTCTCCTGGTACCAACAGTATCCAGGCAAA |
| | | sequence | GCCCCCAAACTCATGATTTATGATGTCAGTGAGCGGCCCTCAGG |
| | | | GGTTTCTAATCGCTTCTCTGGCTCCAAGTCTGGCAACACGGCCT |
| | | | CCCTGACAATCTCTGGGCTCCAGGCTGAGGACGAGGGTGATTAT |
| | | | TACTGCTGCTCATATGCAGCTAATAGCAATGTGGTATTCGGCGG |
| | | | AGGGACCAAACTGTCCGTCCTA |
| 1020 | YANG-21081 | VL domain | QSALTQPASVSGSPGQSITISCTGTSSDVGGYNYVSWYQQYPGK |
| | | amino | APKLMIYDVSERPSGVSNRFSGSKSGNTASLTISGLQAEDEGDY |
| | | acid | YCCSYAANSNVVFGGGTKLSVL |
| | | sequence | |
| 1021 | YANG-21081 | LCDR1 | SSDVGGYNY |
| 1022 | YANG-21081 | LCDR2 | DVS |
| 1023 | YANG-21081 | LCDR3 | CSYAANSNVV |
| 1024 | YANG-2109 | VH domain | CAGGTCCAGCTGGTGCAGTCTGGGGCTGAGGTGAAGAAGCCTGG |
| | | nucleic | GTCCTCGGTGAAGGTCTCCTGTAAGGCTTCTGGAGGCACCTTCA |
| | | acid | GCGGTTATACTATCAGCTGGATTCGACAGGCCCCTGGACAGGGA |
| | | sequence | CTTGAGTGGATGGGAGGGATCATCCCTAGCTTTGGCTCATTAAA |
| | | | CTATGGACAGAAGTTCCAGGACAGAGTCTCGATTACCACGGACG |
| | | | AATTAAAGAGCACTGTGTACATGGAGCTGAGCAGCCTGAATTCT |
| | | | GAGGACACGGCCATATATTATTGTACGAGAGAATCAATAACTTC |
| | | | GGGCTACTATTACGGTATGGCCGTCTGGGGCCAAGGGACCACGG |
| | | | TCACCGTCTCCTCA |
| 1025 | YANG-2109 | VH domain | QVQLVQSGAEVKKPGSSVKVSCKASGGTFSGYTISWIRQAPGQG |
| | | amino | LEWMGGIIPSFGSLNYGQKFQDRVSITTDELKSTVYMELSSLNS |
| | | acid | EDTAIYYCTRESITSGYYYGMAVWGQGTTVTVSS |
| | | sequence | |
| 1026 | YANG-2109 | HCDR1 | GGTFSGYT |
| 1027 | YANG-2109 | HCDR2 | IIPSFGSL |
| 1028 | YANG-2109 | HCDR3 | TRESITSGYYYGMAV |
| 1029 | YANG-2109 | VL domain | GACATCCAGATGACCCAGTCTCCATCCTCCCTGTCTGCATCTGT |
| | | nucleic | AGGAGACAGAGTCACCATCACTTGCCGGGCAAGTCAGGGCATTA |
| | | acid | GAGATGATTTAGGCTGGTTTCAGCAGAAACCAGGGAAAGCCCCT |
| | | sequence | AAGCGCCTGATCTCTGCTGCATCCAGTTTGCAAAGTGGAGTCCC |
| | | | ATCAAGGTTCAGCGGCAGTGGATCTGGGACAGAATTCACTCTCA |
| | | | CAATCAGCAGCCTGCAGCCTGAAGATTTTGCAACTTATTACTGT |
| | | | CTACAGCATAATAGTTACCCGCTCACTTTCGGCGGAGGGACCAA |
| | | | GGTGGAGATCAGA |
| 1030 | YANG-2109 | VL domain | DIQMTQSPSSLSASVGDRVTITCRASQGIRDDLGWFQQKPGKAP |
| | | amino | KRLISAASSLQSGVPSRFSGSGSGTEFTLTISSLQPEDFATYYC |
| | | acid | LQHNSYPLTFGGGTKVEIR |
| | | sequence | |
| 1031 | YANG-2109 | LCDR1 | QGIRDD |
| 1032 | YANG-2109 | LCDR2 | AAS |
| 1033 | YANG-2109 | LCDR3 | LQHNSYPLT |
| 1034 | YANG-2110 | VH domain | GAGGTGCAGCTGGTGGAGTCTGGAGGAGGCTTGATCCAGCCTGG |
| | | nucleic | GGGGTCCCTGAGACTCTCCTGTGCAGCCTCTGGGTTCATCGTCA |
| | | acid | GTCGCAATTATATGAGTTGGGTCCGCCAGGCTCCAGGGAAGGGG |
| | | sequence | CTGGACTGGGTCTCAGTTATTTATAGCGGTGGCAGCACATACTA |
| | | | CGCAGACTCCGTGAAGGGCCGATTTACCATCTCCAGAGACAATT |
| | | | CCAAGAACACGCTGTATCTTCAAATGAACAGCCTGAGAGCCGAG |
| | | | GACACGGCCGTCTATTACTGTGCGAGGTCTATAGCAGTGGCTGG |
| | | | TGAGGGGCTTGACTCCTGGGGCCAGGGAACCCTGGTCACCGTCT |
| | | | CCTCA |
| 1035 | YANG-2110 | VH domain | EVQLVESGGGLIQPGGSLRLSCAASGFIVSRNYMSWVRQAPGKG |
| | | amino | LDWVSVIYSGGSTYYADSVKGRFTISRDNSKNTLYLQMNSLRAE |
| | | acid | DTAVYYCARSIAVAGEGLDSWGQGTLVTVSS |
| | | sequence | |
| 1036 | YANG-2110 | HCDR1 | GFIVSRNY |
| 1037 | YANG-2110 | HCDR2 | IYSGGST |
| 1038 | YANG-2110 | HCDR3 | ARSIAVAGEGLDS |
| 1039 | YANG-2110 | VL domain | GAAATTGTAATGACACAGTCTCCAGCCACCCTGTCTTTGTCTCC |
| | | nucleic | AGGGGACAGAGCCACCCTCTCCTGCAGGGCCAGTCAGAGTGTTA |
| | | acid | GCAACATCTTCATATCCTGGTACCAGCAGAAACCTGGGCAGGCT |
| | | sequence | CCCAGGCTCCTCATTTATGGTGCATCCATCAGGGCCACTGACAT |
| | | | CCCAGCCAGGTTCAGTGGCAGTGGGTCTGGGACAGACTTCACTC |
| | | | TCACCATCAGCAGCCTGCAGCCTGAAGATTTTGCAATTTATTTC |
| | | | TGTCAGCAGGATTATAACTTACCGCTCACTTTCGGCGGAGGGAC |
| | | | CAAGGTGGAGATCAAA |
| 1040 | YANG-2110 | VL domain | EIVMTQSPATLSLSPGDRATLSCRASQSVSNIFISWYQQKPGQA |
| | | amino | PRLLIYGASIRATDIPARFSGSGSGTDFTLTISSLQPEDFAIYF |
| | | acid | CQQDYNLPLTFGGGTKVEIK |
| | | sequence | |
| 1041 | YANG-2110 | LCDR1 | QSVSNIF |
| 1042 | YANG-2110 | LCDR2 | GAS |
| 1043 | YANG-2110 | LCDR3 | QQDYNLPLT |
| 1044 | YANG-2111 | VH domain | GGAGTCCAACTGGTGGAGTCTGGGGGAGGCTTGGTACAGCCTGG |
| | | nucleic | CAGGTCCCTGAGACTCTCCTGTGCAGCCTCTGGATTCACCTTTG |
| | | acid | ATGATTATGCCTTACACTGGGTCCGGCAAGTTCCAGGGAAGGGC |
| | | sequence | CTGGAGTGGGTCTCAAGCATTAGTTGGAATAGTGATGACCTAGG |
| | | | CTATGCGGACTCTGTGGAGGGCCGATTCACCATCTCCAGAGACA |
| | | | ACGCCAAGAACTCCCTGTATCTGCAAATGAGCAGTCTGAGAACT |
| | | | GAGGACACGGCCTTGTATTACTGTGTAAAGGATACTAGGGCGCA |
| | | | TTACGATATTTTGGCTGGTTATAGAGGGCTTGACTCTTGGGGCC |
| | | | AGGGAACCCTGGTCACCGTCTCTTCA |
| 1045 | YANG-2111 | VH domain | GVQLVESGGGLVQPGRSLRLSCAASGFTEDDYALHWVRQVPGKG |
| | | amino | LEWVSSISWNSDDLGYADSVEGRETISRDNAKNSLYLQMSSLRT |
| | | acid | EDTALYYCVKDTRAHYDILAGYRGLDSWGQGTLVTVSS |
| | | sequence | |
| 1046 | YANG-2111 | HCDR1 | GFTEDDYA |
| 1047 | YANG-2111 | HCDR2 | ISWNSDDL |
| 1048 | YANG-2111 | HCDR3 | VKDTRAHYDILAGYRGLDS |
| 1049 | YANG-2111 | VL domain | GATATTGTGATGACTCAGTCTCCACTCTCCCTGCCCGTCACCCC |
| | | nucleic | TGGAGAGCCGGCCTCCATCTCCTGCAGGTCTACTCAGAGCCTCC |
| | | acid | TACATAGTAATGGATACAACTTTTTGGATTGGTACCTGCAGAAG |
| | | sequence | CCAGGGCAGTCTCCACAACTCCTGATCTATTTGGGTTCTAATCG |
| | | | GGCCTCCGGGGTCCCTGACAGGTTCAGTGGCAGTGGATCAGGCA |
| | | | CAGATTTTTCACTGAAAATCAGCAGAGTGGAGGCTGAGGATATT |
| | | | GGAATTTATTACTGCATACAAGGTCTGCAAACTCCTATCACCTT |
| | | | CGGCCAAGGGACACGACTGGAGATTAAA |
| 1050 | YANG-2111 | VL domain | DIVMTQSPLSLPVTPGEPASISCRSTQSLLHSNGYNFLDWYLQK |
| | | amino | PGQSPQLLIYLGSNRASGVPDRESGSGSGTDESLKISRVEAEDI |
| | | acid | GIYYCIQGLQTPITFGQGTRLEIK |
| | | sequence | |
| 1051 | YANG-2111 | LCDR1 | QSLLHSNGYNE |
| 1052 | YANG-2111 | LCDR2 | LGS |
| 1053 | YANG-2111 | LCDR3 | IQGLQTPIT |
| 1054 | YANG-2111a | VH domain | GAAGTGGAGCTGGTGGAGTCTGGGGGAGGCTTGGTACAGCCTGG |
| | | nucleic | CAGGTCCCTGAGACTCTCCTGTGCTGCCTCTGGCTTCATCTTTG |
| | | acid | ATGATTATGCCTTACACTGGGTCCGGCAAATTCCAGGGAAGGGC |
| | | sequence | CTGGAGTGGGTCTCAAGTATTAGTTGGAATAGTGATGACGTAGG |
| | | | CTATGCGGACTCTGTGGAGGGCCGATTCACCATCTCCAGAGACA |
| | | | ACGCCAAGAACTCCCTGTATCTGCAAATGAGCAGTCTGAGAGCT |
| | | | GAGGACACGGCCTTATATTACTGTGTAAAGGATGTTAGGGCTCA |
| | | | TTACGATATTTTGGCTGCTTATAGAGGACTTGACTATTGGGGCC |
| | | | AGGGAACCCTGGTCACCGTCTCCTCA |
| 1055 | YANG-2111a | VH domain | EVELVESGGGLVQPGRSLRLSCAASGFIFDDYALHWVRQIPGKG |
| | | amino | LEWVSSISWNSDDVGYADSVEGRETISRDNAKNSLYLQMSSLRA |
| | | acid | EDTALYYCVKDVRAHYDILAAYRGLDYWGQGTLVTVSS |
| | | sequence | |
| 1056 | YANG-2111a | HCDR1 | GFIFDDYA |
| 1057 | YANG-2111a | HCDR2 | ISWNSDDV |
| 1058 | YANG-2111a | HCDR3 | VKDVRAHYDILAAYRGLDY |
| 1059 | YANG-2111a | VL domain | GATGTTGTGATGACTCAGTCTCCGCTCTCCCTGCCCGTCACCCC |
| | | nucleic | TGGAGAGCCGGCCTCCATCTCCTGCAGGTCTAGTCAGAGCCTCC |
| | | acid | TGCATAGCAATGGATACAACTATTTGGATTGGTACCTGCAGAAG |
| | | sequence | CCAGGGCAGTCTCCACAACTCCTGATCTATTTGGGTTCTAATCG |
| | | | GGCCTCCGGGGTCCCTGACAGGTTCAGTGGCAGTGGATCAGGCA |
| | | | CAGATTTTACACTGAAGATCAGTAGAATGGAGGCTGAGGATGTT |
| | | | GGGGTTTATTACTGCATGCAAGGTCTACAAACTCCTATCACCTT |
| | | | CGGCCAAGGGACACGACTGGAGATTAAG |
| 1060 | YANG-2111a | VL domain | DVVMTQSPLSLPVTPGEPASISCRSSQSLLHSNGYNYLDWYLQK |
| | | amino | PGQSPQLLIYLGSNRASGVPDRFSGSGSGTDFTLKISRMEAEDV |
| | | acid | GVYYCMQGLQTPITFGQGTRLEIK |
| | | sequence | |
| 1061 | YANG-2111a | LCDR1 | QSLLHSNGYNY |
| 1062 | YANG-2111a | LCDR2 | LGS |
| 1063 | YANG-2111a | LCDR3 | MQGLQTPIT |
| 1064 | YANG-2111b | VH domain | GAAGTGCAACTGGTGGAGTCTGGGGGAGGCTTGGTACAGCCTGG |
| | | nucleic | CAGGTCCCTGAGACTCTCCTGTGCAGCCTCTGGCTTCATCTTTG |
| | | acid | ATGATTATGCCTTACACTGGGTCCGGCAAGTTCCAGGGAAGGGC |
| | | sequence | CTGGAGTGGGTCTCAAGTATTAGTTGGAATAGTGATGACATAGG |
| | | | CTATGCGGACTCTGTGGAGGGCCGATTCACCATCTCCAGAGACA |
| | | | ACGCCAAGAACTCCCTGTATCTGCAAATGAGTAGTCTGAGAGCT |
| | | | GAGGACACGGCCTTATATTACTGTGTAAAGGATGTTAGGGCGCA |
| | | | TTACGATATTTTGGCTGCTTATAGAGGACTTGACTATTGGGGCC |
| | | | AGGGAACCCTGGTCACCGTCTCCTCA |
| 1065 | YANG-2111b | VH domain | EVQLVESGGGLVQPGRSLRLSCAASGFIFDDYALHWVRQVPGKG |
| | | amino | LEWVSSISWNSDDIGYADSVEGRFTISRDNAKNSLYLQMSSLRA |
| | | acid | EDTALYYCVKDVRAHYDILAAYRGLDYWGQGTLVTVSS |
| | | sequence | |
| 1066 | YANG-2111b | HCDR1 | GFIFDDYA |
| 1067 | YANG-2111b | HCDR2 | ISWNSDDI |
| 1068 | YANG-2111b | HCDR3 | VKDVRAHYDILAAYRGLDY |
| 1069 | YANG-2111b | VL domain | GATATTGTGATGACTCAGTCTCCACTCTCCCTGCCCGTCACCCC |
| | | nucleic | TGGAGAGCCGGCCTCCATCTCCTGCAGGTCTAGTCAGAGCCTCC |
| | | acid | TGCATAGCAATGGATACAACTATTTGGATTGGTACCTGCAGAAG |
| | | sequence | CCAGGGCAGTCTCCACAACTCCTGATCTATTTGGGTTCTAATCG |
| | | | GGCCTCCGGGGTCCCTGACAGGTTCAGTGTCAGTGGATCAGGCA |
| | | | CAGATTTTACACTGAAAATCAGCAGAGTGGCGGCTGAGGATGTT |
| | | | GGGGTTTATTACTGCATGCAAGGTCTACAAACTCCTATCACCTT |
| | | | CGGCCAAGGGACACGACTGGAGATTACA |
| 1070 | YANG-2111b | VL domain | DIVMTQSPLSLPVTPGEPASISCRSSQSLLHSNGYNYLDWYLQK |
| | | amino | PGQSPQLLIYLGSNRASGVPDRFSVSGSGTDFTLKISRVAAEDV |
| | | acid | GVYYCMQGLQTPITFGQGTRLEIT |
| | | sequence | |
| 1071 | YANG-2111b | LCDR1 | QSLLHSNGYNY |
| 1072 | YANG-2111b | LCDR2 | LGS |
| 1073 | YANG-2111b | LCDR3 | MQGLQTPIT |
| 1074 | YANG-2112 | VH domain | CAGGTCCAACTGGTACAGTCTGGGGCTGAAGTGAAGAAGCCTGG |
| | | nucleic | GGCCTCAGTGAAGGTCTCCTGCAAGGTTTCCGGGTTCACCCTCA |
| | | acid | CTCACTTATCCATTCACTGGGTGCGACAGGCTCCTGGAAGAGGA |
| | | sequence | CTTGAGTGGATGGGAGGTTTTGATCCTGTGGATGGTCAAACAGT |
| | | | CTACGCACAGAACTTCCAGGGCAGAGTCACCATGACCGAGGACA |
| | | | CATCTACAGACACAGCCAACATGGACCTGAACAACCTGAAATCT |
| | | | GAGGACACGGCCGTTTATTACTGTGCGACGGGTGTAGTAGTACC |
| | | | AGCTGCCCCTTACTACTACTCCTCCGGAATGGACGTCTGGGGCC |
| | | | AAGGGACCACGGTCACCGTCTCCTCA |
| 1075 | YANG-2112 | VH domain | QVQLVQSGAEVKKPGASVKVSCKVSGFTLTHLSIHWVRQAPGRG |
| | | amino | LEWMGGFDPVDGQTVYAQNFQGRVTMTEDTSTDTANMDLNNLKS |
| | | acid | EDTAVYYCATGVVVPAAPYYYSSGMDVWGQGTTVTVSS |
| | | sequence | |
| 1076 | YANG-2112 | HCDR1 | GFTLTHLS |
| 1077 | YANG-2112 | HCDR2 | FDPVDGQT |
| 1078 | YANG-2112 | HCDR3 | ATGVVVPAAPYYYSSGMDV |
| 1079 | YANG-2112 | VL domain | CAGTCTGTGCTGACTCAGCCACCCTCAACGTCTGGGACCCCCGG |
| | | nucleic | GCAGAGGGTCACCATCTCTTGCTCTGGAAGCAGCTCCAACATCG |
| | | acid | CAAAAAATTATGTATACTGGTACCAACAACTCCCAGGAACGGCC |
| | | sequence | CCCAAACTCCTCATCTATAGGACTAATCAGCGGCCCTCTGGGGT |
| | | | CCCTGACCGATTCTCTGGCTCCAGGTCTGGCACCTCAGCCTCCC |
| | | | TGGCCATCAGTGGGCTCCGGTCCGAGGATGAGGCTACTTATTAC |
| | | | TGTGCAACATGGGATGACACCCTGAGTGTGATATTCGGCGGAGG |
| | | | GACCAACCTGACCGTCCTG |
| 1080 | YANG-2112 | VL domain | QSVLTQPPSTSGTPGQRVTISCSGSSSNIAKNYVYWYQQLPGTA |
| | | amino | PKLLIYRTNQRPSGVPDRESGSRSGTSASLAISGLRSEDEATYY |
| | | acid | CATWDDTLSVIFGGGTNLTVL |
| | | sequence | |
| 1081 | YANG-2112 | LCDR1 | SSNIAKNY |
| 1082 | YANG-2112 | LCDR2 | RTN |
| 1083 | YANG-2112 | LCDR3 | ATWDDTLSVI |
| 1084 | YANG-2201 | VH domain | CAGGAGCAGCTGGTGGAGTCTGGGGGAGGCGTGGTCCAGCCTGG |
| | | nucleic | GATGTCCCTGAGACTCTCCTGTGCAGCCTCTGGATTCACCTTCA |
| | | acid | GTAACTATGGCATGCACTGGGTCCGCCAGTCTCCCGGCAAGGGG |
| | | sequence | CTGGAATGGGTGACTTTTATATCATATGATGGAAATAATGAATA |
| | | | CTATGTAGACTCCGTGAAGGGCCGATTCACCATCTCCAGAGACA |
| | | | ATTCCAAGAACACGCTGTTTCTGCAAATGAACAGCCTGAGAGCT |
| | | | GAGGACGCGGCTGTGTATTACTGTGCGAAAGATCGTGCATTGAA |
| | | | CCTTTACTACTTTGATTCCTGGGGCCCGGGAACCCTGGTCACCG |
| | | | TCTCCTCA |
| 1085 | YANG-2201 | VH domain | QEQLVESGGGVVQPGMSLRLSCAASGFTFSNYGMHWVRQSPGKG |
| | | amino | LEWVTFISYDGNNEYYVDSVKGRFTISRDNSKNTLFLQMNSLRA |
| | | acid | EDAAVYYCAKDRALNLYYFDSWGPGTLVTVSS |
| | | sequence | |
| 1086 | YANG-2201 | HCDR1 | GFTFSNYG |
| 1087 | YANG-2201 | HCDR2 | ISYDGNNE |
| 1088 | YANG-2201 | HCDR3 | AKDRALNLYYFDS |
| 1089 | YANG-2201 | VL domain | GATATTGTGATGACTCAGCCTCCACTCTCCCTGCCCGTCACTCC |
| | | nucleic | TGGAGAGCCGGCCTCCATCTCCTGCAGGTCTAGTCAGAGCCTCC |
| | | acid | TGCATAATAATGCCTACAACCATTIGAATTGGTATCTGCAGAAG |
| | | sequence | CCAGGGCAGTCTCCACAGTTCCTGATCTATTTGGGTTCTAATCG |
| | | | GGCCTCCGGGGTCCCTGACAGGTTCAGTGGCAGTGGATCAGGCA |
| | | | CAGATTTTACACTGAAAATCAGCAGAGTGGAGGCTGAGGATGTT |
| | | | GGGGTTTATTACTGCATGCAATCTCTACAAATTCCGATCACCTT |
| | | | CGGCCAAGGGACACGACTGGAAATTAAA |
| 1090 | YANG-2201 | VL domain | DIVMTQPPLSLPVTPGEPASISCRSSQSLLHNNAYNHLNWYLQK |
| | | amino | PGQSPQFLIYLGSNRASGVPDRFSGSGSGTDFTLKISRVEAEDV |
| | | acid | GVYYCMQSLQIPITFGQGTRLEIK |
| | | sequence | |
| 1091 | YANG-2201 | LCDR1 | QSLLHNNAYNH |
| 1092 | YANG-2201 | LCDR2 | LGS |
| 1093 | YANG-2201 | LCDR3 | MQSLQIPIT |
| 1094 | YANG-2202 | VH domain | GAGGTGCAATTGGTGGAGTCTGGGGGAGGCTTGGTTCAGCCGGG |
| | | nucleic | GGGGTCCCTGAGACTCTCCTGTGCAGCCTCTGGATTCACCTTTA |
| | | acid | GCAGTTTTGCCATGAGTTGGGTCCGCCAGGCTCCAGGGAAGGGA |
| | | sequence | CTTGAGTGGGTCGCAGCCTTCAGTGGTCGTGGTGCGATTACACA |
| | | | CTACACAGACTCCGTGAAGGGCCGGTTCACCATCTCCAGAGACA |
| | | | ATTCCAAGAACACGCTGTATCTGCAAATGAACAGCCTGAGAGCC |
| | | | GAGGACACGGCCGTATATTACTGTGCGACTTATAATTGGAACCC |
| | | | CTACTACTTTGACTTCTGGGGCCAGGGAACCCTGGTCACCGTCT |
| | | | CCTCA |
| 1095 | YANG-2202 | VH domain | EVQLVESGGGLVQPGGSLRLSCAASGFTFSSFAMSWVRQAPGKG |
| | | amino | LEWVAAFSGRGAITHYTDSVKGRFTISRDNSKNTLYLQMNSLRA |
| | | acid | EDTAVYYCATYNWNPYYFDFWGQGTLVTVSS |
| | | sequence | |
| 1096 | YANG-2202 | HCDR1 | GFTFSSFA |
| 1097 | YANG-2202 | HCDR2 | FSGRGAIT |
| 1098 | YANG-2202 | HCDR3 | ATYNWNPYYFDF |
| 1099 | YANG-2202 | VL domain | GACATCCAGATGACCCAGTCTCCATCTTCCGTGTCTGCATCTAT |
| | | nucleic | AGGAGACAGAGTCACCATCACCTGTCGGGCGAGTCAGGATATTA |
| | | acid | ATATCTGGTTGGCCTGGTATCAGCAGAAACCAGGGAAAGCCCCT |
| | | sequence | AATCTCCTGATCTTTGATACATCCAATTTACAGAGTGGGGTCCC |
| | | | ATCAAGGTTCAGTGGCAGTGGATCTGGGTCAGATTTCACTCTCA |
| | | | CCATCAGCAGCCTGCAGCCTGAAGATTTTGCAACTTACTATTGT |
| | | | CAACAGGGTAACAGTTTCCCTCGGACCTTCGGCCAGGGGACACG |
| | | | ACTGGAAATTAAA |
| 1100 | YANG-2202 | VL domain | DIQMTQSPSSVSASIGDRVTITCRASQDINIWLAWYQQKPGKAP |
| | | amino | NLLIFDTSNLQSGVPSRFSGSGSGSDFTLTISSLQPEDFATYYC |
| | | acid | QQGNSFPRTFGQGTRLEIK |
| | | sequence | |
| 1101 | YANG-2202 | LCDR1 | QDINIW |
| 1102 | YANG-2202 | LCDR2 | DTS |
| 1103 | YANG-2202 | LCDR3 | QQGNSFPRT |
| 1104 | YANG-2203 | VH domain | CAGGTGCAACTGGTGCAGTCTGGGGCTGAGGTGAAGCAGCCTGG |
| | | nucleic | GGCCTCAGTGAAGGTTTCCTGCAGGGCATCTGGATACGCCCTCA |
| | | acid | CCAGCTTCTATGTTCACTGGGTGCGACAGGCCCCTGGACAAGGG |
| | | sequence | CTTGAGTGGATGGGCATAATCAAACCCAGTGGTGGTGACACAAT |
| | | | CTACGCTCAGAATCTCCAGGGCAGAGTCACCATGACCAGGGACA |
| | | | CGTCCACGAACACATTCTACGTGGAACTGAGCAGCCTGAGATCT |
| | | | GAGGACACGGCCCTATATTACTGTGCGATCAGTGGGAACACTAG |
| | | | GGCTTTTGAAATCTGGGGCCAAGGGACAATGGTCACCGTCTCTT |
| | | | TA |
| 1105 | YANG-2203 | VH domain | QVQLVQSGAEVKQPGASVKVSCRASGYALTSFYVHWVRQAPGQG |
| | | amino | LEWMGIIKPSGGDTIYAQNLQGRVTMTRDTSTNTFYVELSSLRS |
| | | acid | EDTALYYCAISGNTRAFEIWGQGTMVTVSL |
| | | sequence | |
| 1106 | YANG-2203 | HCDR1 | GYALTSFY |
| 1107 | YANG-2203 | HCDR2 | IKPSGGDT |
| 1108 | YANG-2203 | HCDR3 | AISGNTRAFEI |
| 1109 | YANG-2203 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTTTGTATCCTGGTACCAGCAGCTCCCAGGAATGGTC |
| | | sequence | CCCAAACTCCTCATTTATGCCAATAATAAGCGACCCTCAGGGAT |
| | | | TCCTGACCGATTCTCTGGCTCCAGGTCTGGCACGTCAGCCACCC |
| | | | TGGGCATCACCGGACTCCAGACTGGGGACGAGGCCGATTATTAC |
| | | | TGCGGAACTTGGGATAGTAGTCGGAGTGCTGTGCTTTTCGGCGG |
| | | | AGGGACCAAGATGACCGTCCTA |
| 1110 | YANG-2203 | VL domain | QSVLTQPPSVSAAPGQKVTISCSGSSSNIGNNFVSWYQQLPGMV |
| | | amino | PKLLIYANNKRPSGIPDRESGSRSGTSATLGITGLQTGDEADYY |
| | | acid | CGTWDSSRSAVLFGGGTKMTVL |
| | | sequence | |
| 1111 | YANG-2203 | LCDR1 | SSNIGNNE |
| 1112 | YANG-2203 | LCDR2 | ANN |
| 1113 | YANG-2203 | LCDR3 | GTWDSSRSAVL |
| 1114 | YANG-2203a | VH domain | CAGGTGCAACTGGTGCAGTCTGGGGCTGAGGTGAAGCAGCCTGG |
| | | nucleic | GGCCTCAGTGAAGGTTTCCTGCAGGGCATCTGGATACGCCCTCA |
| | | acid | CCAGCTTCTATGTTCACTGGGTGCGACAGGCCCCTGGACAAGGA |
| | | sequence | CTTGAGTGGATGGGCATAATCAAACCCAGTGGTGGTGACACAAT |
| | | | CTACGCTCAGAATCTCCAGGGCAGAGTCACCATGACCAGGGACA |
| | | | CGTCCACGAACACATTCTACGTGGAACTGAGCAGCCTGAGATCT |
| | | | GAGGACACGGCCCTATATTACTGTGCGATCAATGGGGACACTAG |
| | | | GGCTTTTGAAATCTGGGGCCAAGGGACAATGGTCACCGTCTCTT |
| | | | TA |
| 1115 | YANG-2203a | VH domain | QVQLVQSGAEVKQPGASVKVSCRASGYALTSFYVHWVRQAPGQG |
| | | amino | LEWMGIIKPSGGDTIYAQNLQGRVTMTRDTSINTFYVELSSLRS |
| | | acid | EDTALYYCAINGDTRAFEIWGQGTMVTVSL |
| | | sequence | |
| 1116 | YANG-2203a | HCDR1 | GYALTSFY |
| 1117 | YANG-2203a | HCDR2 | IKPSGGDT |
| 1118 | YANG-2203a | HCDR3 | AINGDTRAFEI |
| 1119 | YANG-2203a | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTTTGTATCCTGGTACCAGCAGCTCCCAGGAATGGTC |
| | | sequence | CCCAAACTCCTCATTTATGCCAATAATAAGCGACCCTCAGGGAT |
| | | | TCCTGACCGATTCTCTGGCTCCAGGTCTGGCACGTCAGCCACCC |
| | | | TGGGCATCACCGGACTCCAGACTGGGGACGAGGCCGATTATTAC |
| | | | TGCGGAACTTGGGATAGTAGTCGGAGTGCTGTGCTTTTCGGCGG |
| | | | AGGGACCAAGATGACCGTCCTA |
| 1120 | YANG-2203a | VL domain | QSVLTQPPSVSAAPGQKVTISCSGSSSNIGNNFVSWYQQLPGMV |
| | | amino | PKLLIYANNKRPSGIPDRFSGSRSGTSATLGITGLQTGDEADYY |
| | | acid | CGTWDSSRSAVLFGGGTKMTVL |
| | | sequence | |
| 1121 | YANG-2203a | LCDR1 | SSNIGNNF |
| 1122 | YANG-2203a | LCDR2 | ANN |
| 1123 | YANG-2203a | LCDR3 | GTWDSSRSAVL |
| 1124 | YANG-2203b | VH domain | CAGGTGCAACTGGTGCAGTCTGGGGCTGAGGTGAAGCAGCCTGG |
| | | nucleic | GGCCTCAGTGAAGGTTTCCTGCAGGGCATCTGGATACGCCCTCA |
| | | acid | CCAACTTCTATGTTCACTGGGTGCGACAGGCCCCTGGACAAAAA |
| | | sequence | CTTGAGTGGATGGGCATAATCAAACCCAGTGGTGGTGACACAAT |
| | | | CTACGCTCAGAATCTCCAGGGCAGAGTCACCATGACCAGGGACA |
| | | | CGTCCACGAATACATTCTACGTGGAACTGAGCAGCCTGAGATCT |
| | | | GAGGACACGGCCCTATATTACTGTGCGATCAGTGGGAACACTAG |
| | | | GGCTTTTGAAATCTGGGGCCAAGGGACAATGGTCACCGTCTCTT |
| | | | TA |
| 1125 | YANG-2203b | VH domain | QVQLVQSGAEVKQPGASVKVSCRASGYALTNFYVHWVRQAPGQK |
| | | amino | LEWMGIIKPSGGDTIYAQNLQGRVTMTRDTSTNTFYVELSSLRS |
| | | acid | EDTALYYCAISGNTRAFEIWGQGTMVTVSL |
| | | sequence | |
| 1126 | YANG-2203b | HCDR1 | GYALTNFY |
| 1127 | YANG-2203b | HCDR2 | IKPSGGDT |
| 1128 | YANG-2203b | HCDR3 | AISGNTRAFEI |
| 1129 | YANG-2203b | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTTTATATCCTGGTACCAGCAGCTCCCAGGAATGGTC |
| | | sequence | CCCAAACTCCTCATTTATGCCAATAATAAGCGACCCTCAGGGAT |
| | | | TCCTGACCGATTCTCTGGCTCCAGGTCTGGCACGTCAGCCACCC |
| | | | TGGGCATCACCGGACTCCAGACTGGGGACGAGGCCGATTATTTC |
| | | | TGCGGAACTTGGGATAGTAGTCGGAGTGCTGTGCTTTTCGGCGG |
| | | | AGGGACCAAGATGACCGTCCTA |
| 1130 | YANG-2203b | VL domain | QSVLTQPPSVSAAPGQKVTISCSGSSSNIGNNFISWYQQLPGMV |
| | | amino | PKLLIYANNKRPSGIPDRFSGSRSGTSATLGITGLQTGDEADYF |
| | | acid | CGTWDSSRSAVLFGGGTKMTVL |
| | | sequence | |
| 1131 | YANG-2203b | LCDR1 | SSNIGNNF |
| 1132 | YANG-2203b | LCDR2 | ANN |
| 1133 | YANG-2203b | LCDR3 | GTWDSSRSAVL |
| 1134 | YANG-2203c | VH domain | CAGGTGCAACTGGTGCAGTCTGGGGCTGAGGTGAAGCAGCCTGT |
| | | nucleic | GGCCTCAGTGAAGGTTTCCTGTCGGGCATCTGGTTACGCCCTCA |
| | | acid | CCAGCTTCTATGTTCACTGGGTGCGACAGGCCCCTGGACAAGGG |
| | | sequence | CTTGAGTGGATGGGCATAATCAAACCCAGTGGTGGTGACACAAT |
| | | | CTACGCTCAGAATCTCCAGGGCAGAGTCACCATGACCAGGGACA |
| | | | CGTCCACGAACACATTCTACGTGGAACTGAGCAGCCTGAGATCT |
| | | | GAGGACACGGCCCTATATTACTGTGCGATCAGTGGGAACACTAG |
| | | | GGCTTTTGAAATCTGGGGCCAAGGGACAATGGTCACCGTCTCTT |
| | | | TA |
| 1135 | YANG-2203c | VH domain | QVQLVQSGAEVKQPVASVKVSCRASGYALTSFYVHWVRQAPGQG |
| | | amino | LEWMGIIKPSGGDTIYAQNLQGRVTMTRDTSTNTFYVELSSLRS |
| | | acid | EDTALYYCAISGNTRAFEIWGQGTMVTVSL |
| | | sequence | |
| 1136 | YANG-2203c | HCDR1 | GYALTSFY |
| 1137 | YANG-2203c | HCDR2 | IKPSGGDT |
| 1138 | YANG-2203c | HCDR3 | AISGNTRAFEI |
| 1139 | YANG-2203c | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTTTGTATCCTGGTACCAGCAGCTCCCAGGAATGGTC |
| | | sequence | CCCAAACTCCTCATTTATGCCAATAATAAGCGACCCTCAGGGAT |
| | | | TCCTGACCGATTCTCTGGCTCCAGGTCTGGCACGTCAGCCACCC |
| | | | TGGGCATCACCGGACTCCAGACTGGGGACGAGGCCGATTATTAC |
| | | | TGCGGAACTTGGGATAGTAGTCGGAGTGCTGTGCTTTTCGGCGG |
| | | | AGGGACCAAGATGACCGTCCTA |
| 1140 | YANG-2203c | VL domain | QSVLTQPPSVSAAPGQKVTISCSGSSSNIGNNFVSWYQQLPGMV |
| | | amino | PKLLIYANNKRPSGIPDRFSGSRSGTSATLGITGLQTGDEADYY |
| | | acid | CGTWDSSRSAVLFGGGTKMTVL |
| | | sequence | |
| 1141 | YANG-2203c | LCDR1 | SSNIGNNE |
| 1142 | YANG-2203c | LCDR2 | ANN |
| 1143 | YANG-2203c | LCDR3 | GTWDSSRSAVL |
| 1144 | YANG-2203d | VH domain | CAGGTGCAACTGGTGCAGTCTGGGGCTGAGGTGAAGCAGCCTGG |
| | | nucleic | GGCCTCAGTGAAGGTTTCCTGTAGGGCATCTGGATACGCCCTCA |
| | | acid | CCAGCTTCTATGTTCACTGGGTGCGACAGGCCCCTGGACAAGGG |
| | | sequence | CTTGAGTGGATGGGCATAATCAAACCCAGTGGTGGTGACACAAT |
| | | | CTACGCTCAGAATCTCCAGGGCAGAGTCACCATGACCAGGGACA |
| | | | CGTCCACGAACACATTCTACGTGGAACTGAGCAGCCTGAGATCT |
| | | | GAGGACACGGCCCTATATTACTGTGCGATCAGTGGGAACACTAG |
| | | | GGCTTTTGAAATCTGGGGCCAAGGGACAATGGTCACCGTCTCTT |
| | | | TA |
| 1145 | YANG-2203d | VH domain | QVQLVQSGAEVKQPGASVKVSCRASGYALTSFYVHWVRQAPGQG |
| | | amino | LEWMGIIKPSGGDTIYAQNLQGRVTMTRDTSTNTFYVELSSLRS |
| | | acid | EDTALYYCAISGNTRAFEIWGQGTMVTVSL |
| | | sequence | |
| 1146 | YANG-2203d | HCDR1 | GYALTSFY |
| 1147 | YANG-2203d | HCDR2 | IKPSGGDT |
| 1148 | YANG-2203d | HCDR3 | AISGNTRAFEI |
| 1149 | YANG-2203d | VL domain | CAGTCTGTGTTGACGCAGCCGCCCTCAGTGTCTGCGGCCCCAGG |
| | | nucleic | ACAGAAGGTCACCATCTCCTGTTCTGGAAGCAGCTCCAACATTG |
| | | acid | GGAATAATTTTGTCTCCTGGTACCAGCAGCTCCCAGGAATGGTC |
| | | sequence | CCCAAACTCCTCATTTATGCCAATAATAAGCGACCCTCAGGGAT |
| | | | TCCTGACCGATTCTCTGGCTCCAGGTCTGGCACGTCAGCCACCC |
| | | | TGGGCATCACCGGACTCCAGACTGGGGACGAGGCCGATTATTAC |
| | | | TGCGGAACTTGGGATAGTAGTCGGAGTGTTGTGCTTTTCGGCGG |
| | | | AGGGACCAAGATGACCGTCCTA |
| 1150 | YANG-2203d | VL domain | QSVLTQPPSVSAAPGQKVTISCSGSSSNIGNNFVSWYQQLPGMV |
| | | amino | PKLLIYANNKRPSGIPDRESGSRSGTSATLGITGLQTGDEADYY |
| | | acid | CGTWDSSRSVVLFGGGTKMTVL |
| | | sequence | |
| 1151 | YANG-2203d | LCDR1 | SSNIGNNE |
| 1152 | YANG-2203d | LCDR2 | ANN |
| 1153 | YANG-2203d | LCDR3 | GTWDSSRSVVL |
| 1154 | YANG-2203e | VH domain | CAGGTGCAACTGGTGCAGTCTGGGGCTGAGGTGAAGCAGCCTGG |
| | | nucleic | GGCCTCAGTGAAGGTTTCCTGCAAGGCATCTGGATACGCCCTCA |
| | | acid | CCAGCTTCTATGTTCACTGGGTGCGACAGGCCCCTGGACAAGGG |
| | | sequence | CTTGAGTGGATGGGCATAATCAAACCTAGTGGTGGTAACACAAT |
| | | | CTACGCTCAGAATTTCCAGGGCAGAGTCACCATGACCAGGGACA |
| | | | CGTCCACGAACACATTCTACGTGGAACTGAACAGCCTGAGATCT |
| | | | GAGGACACGGCCCTATATTACTGTGCGATCAGTGGGAACACTAG |
| | | | GGCTTTTGAAATCTGGGGCCAGGGGACAATGGTCACCGTCTCTT |
| | | | CA |
| 1155 | YANG-2203e | VH domain | QVQLVQSGAEVKQPGASVKVSCKASGYALTSFYVHWVRQAPGQG |
| | | amino | LEWMGIIKPSGGNTIYAQNFQGRVTMTRDTSTNTFYVELNSLRS |
| | | acid | EDTALYYCAISGNTRAFEIWGQGTMVTVSS |
| | | sequence | |
| 1156 | YANG-2203e | HCDR1 | GYALTSFY |
| 1157 | YANG-2203e | HCDR2 | IKPSGGNT |
| 1158 | YANG-2203e | HCDR3 | AISGNTRAFEI |
| 1159 | YANG-2203e | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTATGTATCCTGGTACCAGCAGTTCCCAGGAATAGTC |
| | | sequence | CCCAAACTCCTCATTTATGCCAATAATAAGCGACCCTCAGGGAT |
| | | | TCCTGACCGATTCTCTGGCTCCAGGTCTGGCACGTCAGCCACCC |
| | | | TGGACATCACCGGACTCCAGACTGGGGACGAGGCCGATTATTAC |
| | | | TGCGGAACTTGGGATAGCAGCCGGAGTGCTGTGCTTTTCGGCGG |
| | | | AGGGACCAAGATGACCGTCCTA |
| 1160 | YANG-2203e | VL domain | QSVLTQPPSVSAAPGQKVTISCSGSSSNIGNNYVSWYQQFPGIV |
| | | amino | PKLLIYANNKRPSGIPDRESGSRSGTSATLDITGLQTGDEADYY |
| | | acid | CGTWDSSRSAVLFGGGTKMTVL |
| | | sequence | |
| 1161 | YANG-2203e | LCDR1 | SSNIGNNY |
| 1162 | YANG-2203e | LCDR2 | ANN |
| 1163 | YANG-2203e | LCDR3 | GTWDSSRSAVL |
| 1164 | YANG-2203f | VH domain | CAGGTGCAACTGGTGCAGTCTGGGGCTGAGGTGAAGCAGCCGGG |
| | | nucleic | GGCCTCAGTGAAGGTCTCCTGCAAGGCATCTGGATACGCCCTCA |
| | | acid | CCAGCTTCTATGTTCACTGGGTGCGACAGGCCCCTGGACAAGGG |
| | | sequence | CTTGAGTGGATGGGCATAATCAAACCTAGTGGTGGTAACACAAT |
| | | | CTACGCTCAGAATTTCCAGGGCAGAGTCACCATGACCAGGGACA |
| | | | CGTCCACGAACACATTCTACGTGGAACTAAACAGCCTGAGATCT |
| | | | GAGGACACGGCCCTATATTACTGTGCGATCAGTGGGAACACTAG |
| | | | GGCTTTTGAAATCTGGGGCCAAGGGACAATGGTCACCGTCTCTT |
| | | | TA |
| 1165 | YANG-2203f | VH domain | QVQLVQSGAEVKQPGASVKVSCKASGYALTSFYVHWVRQAPGQG |
| | | amino | LEWMGIIKPSGGNTIYAQNFQGRVTMTRDTSTNTFYVELNSLRS |
| | | acid | EDTALYYCAISGNTRAFEIWGQGTMVTVSL |
| | | sequence | |
| 1166 | YANG-2203f | HCDR1 | GYALTSFY |
| 1167 | YANG-2203f | HCDR2 | IKPSGGNT |
| 1168 | YANG-2203f | HCDR3 | AISGNTRAFEI |
| 1169 | YANG-2203f | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTATGTATCCTGGTACCAGCAGCTCCCAGGAATAGTC |
| | | sequence | CCCAAGCTCCTCATTTATGCCAATAATAAGCGACCCTCAGGGAT |
| | | | TCCTGACCGATTCTCTGGCTCCAGGTCTGGCACGTCAGCCACCC |
| | | | TGGGCATCACCGGACTCCAGACTGGGGACGAGGCCGATTATTAC |
| | | | TGCGGAACTTGGGATAGCAGCCGGAGTGCTGTACTTTTCGGCGG |
| | | | AGGGACCAAGATGACCGTCCTA |
| 1170 | YANG-2203£ | VL domain | QSVLTQPPSVSAAPGQKVTISCSGSSSNIGNNYVSWYQQLPGIV |
| | | amino | PKLLIYANNKRPSGIPDRESGSRSGTSATLGITGLQTGDEADYY |
| | | acid | CGTWDSSRSAVLFGGGTKMTVL |
| | | sequence | |
| 1171 | YANG-2203f | LCDR1 | SSNIGNNY |
| 1172 | YANG-2203f | LCDR2 | ANN |
| 1173 | YANG-2203f | LCDR3 | GTWDSSRSAVL |
| 1174 | YANG-2203g | VH domain | CAGGTGCAACTGGTGCAGTCTGGGGCTGAGGTGAAGCAGCCTGG |
| | | nucleic | GGCCTCAGTGAAGGTTTCCTGCAGGGCATCTGGATACGCCCTCA |
| | | acid | CCAGCTTCTATGTTCACTGGGTGCGACAGGCCCCTGGACAAGGG |
| | | sequence | CTTGAGTGGATGGGCATAATCAAACCCAGTGGTGGTGACACAAT |
| | | | CTACGCTCAGAATCTCCAGGGCAGAGTCACCATGACCAGGGACA |
| | | | CGTCCACGAACACATTCTACGTGGAACTGAGCAGCCTGAGATCT |
| | | | GAGGACACGGCCCTTTATTACTGTGCGATCAGTGGGAACACTAG |
| | | | GGCTTTTGAAATCTGGGGCCAAGGGACAATGGTCACCGTCTCTT |
| | | | TA |
| 1175 | YANG-2203g | VH domain | QVQLVQSGAEVKQPGASVKVSCRASGYALTSFYVHWVRQAPGQG |
| | | amino | LEWMGIIKPSGGDTIYAQNLQGRVTMTRDTSTNTFYVELSSLRS |
| | | acid | EDTALYYCAISGNTRAFEIWGQGTMVTVSL |
| | | sequence | |
| 1176 | YANG-2203g | HCDR1 | GYALTSFY |
| 1177 | YANG-2203g | HCDR2 | IKPSGGDT |
| 1178 | YANG-2203g | HCDR3 | AISGNTRAFEI |
| 1179 | YANG-2203g | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTTTGTATCCTGGTACCAGCAGCTCCCAGGAATGGTC |
| | | sequence | CCCAAACTCCTCATTTATGCCAATAATAAGCGACCCTCAGGGAT |
| | | | TCCTGACCGATTCTCTGGCTCCAGGTCTGGCACGTCAGCCACCC |
| | | | TGGGCATCACCGGACTCCAGACTGGGGACGAGGCCGATTATTAC |
| | | | TGCGGAACTTGGGATAGTAGTCGGAGTGCTGTGCTTTTCGGCGG |
| | | | AGGGACCAAGATGACCGTCCTA |
| 1180 | YANG-2203g | VL domain | QSVLTQPPSVSAAPGQKVTISCSGSSSNIGNNFVSWYQQLPGMV |
| | | amino | PKLLIYANNKRPSGIPDRESGSRSGTSATLGITGLQTGDEADYY |
| | | acid | CGTWDSSRSAVLFGGGTKMTVL |
| | | sequence | |
| 1181 | YANG-2203g | LCDR1 | SSNIGNNF |
| 1182 | YANG-2203g | LCDR2 | ANN |
| 1183 | YANG-2203g | LCDR3 | GTWDSSRSAVL |
| 1184 | YANG-2203h | VH domain | CAGGTGCAACTGGTGCAGTCTGGGGCTGAGGTGAAGCAGCCTGG |
| | | nucleic | GGCCTCAGTGAAGGTTTCCTGCAGGGCATCTGGATACGCCCTCA |
| | | acid | CCAGCTTCTATGTTCACTGGGTGCGACAGGCCCCTGGACAAGGA |
| | | sequence | CTTGACTGGATGGGCATAATCAAACCCAGTGGTGGTTATACAAT |
| | | | TTACGCTCAGAATCTCCAGGGCAGAGTCACCATGACCAGGGACA |
| | | | CGTCCACGAACACATTCTACGTGGAACTGAGCAGCCTGAGATCT |
| | | | GAGGACACGGCCCTATATTACTGTGCGATCAGTGGGAACACTAG |
| | | | GGCTTTTGAAATCTGGGGCCAAGGGACAATGGTCACCGTCTCTT |
| | | | TA |
| 1185 | YANG-2203h | VH domain | QVQLVQSGAEVKQPGASVKVSCRASGYALTSFYVHWVRQAPGQG |
| | | amino | LDWMGIIKPSGGYTIYAQNLQGRVTMTRDTSTNTFYVELSSLRS |
| | | acid | EDTALYYCAISGNTRAFEIWGQGTMVTVSL |
| | | sequence | |
| 1186 | YANG-2203h | HCDR1 | GYALTSFY |
| 1187 | YANG-2203h | HCDR2 | IKPSGGYT |
| 1188 | YANG-2203h | HCDR3 | AISGNTRAFEI |
| 1189 | YANG-2203h | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTTTGTATCCTGGTACCAGCAGCTCCCAGGAATGGTC |
| | | sequence | CCCAAACTCCTCATTTATGCCAATAATAAGCGACCCTCAGGGAT |
| | | | TCCTGACCGATTCTCTGGCTCCAGGTCTGGCACGTCAGCCACCC |
| | | | TGGGCATCACCGGACTCCAGACTGGGGACGAGGCCGATTATTAC |
| | | | TGCGGAACTTGGGATAGTAGTCGGAGTGCTGTGCTTTTCGGCGG |
| | | | AGGGACCAAGATGACCGTCCTA |
| 1190 | YANG-2203h | VL domain | QSVLTQPPSVSAAPGQKVTISCSGSSSNIGNNFVSWYQQLPGMV |
| | | amino | PKLLIYANNKRPSGIPDRFSGSRSGTSATLGITGLQTGDEADYY |
| | | acid | CGTWDSSRSAVLFGGGTKMTVL |
| | | sequence | |
| 1191 | YANG-2203h | LCDR1 | SSNIGNNE |
| 1192 | YANG-2203h | LCDR2 | ANN |
| 1193 | YANG-2203h | LCDR3 | GTWDSSRSAVL |
| 1194 | YANG-2203i | VH domain | CAGGTGCAACTGGTGCAGTCTGGGGCTGAGGTGAAGCAGCCTGG |
| | | nucleic | GGCCTCAGTGAAGGTTTCCTGCAGGGCATCTGGATACGCCCTCA |
| | | acid | CCAACTTCTATATTCACTGGGTGCGACAGGCCCCTGGACAAGGG |
| | | sequence | CTTGAGTGGATGGGCATAATCAAACCCAGTGGTGGTGACACAAT |
| | | | CTACGCTCAGAATCTCCAGGGCAGAGTCACCATGACCAGGGACA |
| | | | CGTCCACGGACACATTCTACGTGGAACTGAACAGCCTGAGATCT |
| | | | GAGGACACGGCCCTATATTACTGTGCGATCAGTGGGAACACTAG |
| | | | GGCTTTTGAAATCTGGGGCCAAGGGACAATGGTCACCGTCTCTT |
| | | | TA |
| 1195 | YANG-2203i | VH domain | QVQLVQSGAEVKQPGASVKVSCRASGYALTNFYIHWVRQAPGQG |
| | | amino | LEWMGIIKPSGGDTIYAQNLQGRVTMTRDTSTDTFYVELNSLRS |
| | | acid | EDTALYYCAISGNTRAFEIWGQGTMVTVSL |
| | | sequence | |
| 1196 | YANG-2203i | HCDR1 | GYALTNFY |
| 1197 | YANG-2203i | HCDR2 | IKPSGGDT |
| 1198 | YANG-2203i | HCDR3 | AISGNTRAFEI |
| 1199 | YANG-2203i | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTTTGTATCCTGGTACCAGCAGCTCCCAGGAATGGTC |
| | | sequence | CCCAAACTCCTCATTTATGCCAATAATAAGCGACCCTCAGGGAT |
| | | | TCCTGACCGATTCTCTGGCTCCAGGTCTGGCACGTCAGCCACCC |
| | | | TGGGCATCACCGGACTCCAGACTGGGGACGAGGCCGATTATTAC |
| | | | TGCGGAACTTGGGATAGTAGTCGGAGTGCTGTGCTTTTCGGCGG |
| | | | AGGGACCAAGATGACCGTCCTA |
| 1200 | YANG-2203i | VL domain | QSVLTQPPSVSAAPGQKVTISCSGSSSNIGNNFVSWYQQLPGMV |
| | | amino | PKLLIYANNKRPSGIPDRFSGSRSGTSATLGITGLQTGDEADYY |
| | | acid | CGTWDSSRSAVLFGGGTKMTVL |
| | | sequence | |
| 1201 | YANG-2203i | LCDR1 | SSNIGNNF |
| 1202 | YANG-2203i | LCDR2 | ANN |
| 1203 | YANG-2203i | LCDR3 | GTWDSSRSAVL |
| 1204 | YANG-2203j | VH domain | CAGGTGCAATTGGTGCAGTCTGGGGCTGAGGTGAAGCAGCCTGG |
| | | nucleic | GGCCTCAGTGAAGGTTTCCTGCAGGGCATCTGGATACGCCCTCA |
| | | acid | CCAGCTTCTATGTTCACTGGGTGCGACAGGCCCCTGGACAAGGG |
| | | sequence | CTTGAGTGGATGGGCATAATCAAACCCAGTGGTGGTGACACAAT |
| | | | CTACGCTCAGAATCTCCAGGGCAGAGTCACCATGACCAGGGACA |
| | | | CGTCCACGAACACATTCTACGTGGAACTGAGCAGCCTGAGATCT |
| | | | GAGGACACGGCCCTATATTACTGTGCGATCAGTGGGAACACTAG |
| | | | GGCTTTTGAAATCTGGGGCCAAGGGACAATGGTCACCGTCTCTT |
| | | | TA |
| 1205 | YANG-2203j | VH domain | QVQLVQSGAEVKQPGASVKVSCRASGYALTSFYVHWVRQAPGQG |
| | | amino | LEWMGIIKPSGGDTIYAQNLQGRVTMTRDTSTNTFYVELSSLRS |
| | | acid | EDTALYYCAISGNTRAFEIWGQGTMVTVSL |
| | | sequence | |
| 1206 | YANG-2203j | HCDR1 | GYALTSFY |
| 1207 | YANG-2203j | HCDR2 | IKPSGGDT |
| 1208 | YANG-2203j | HCDR3 | AISGNTRAFEI |
| 1209 | YANG-2203j | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTTTGTATCCTGGTACCAGCAGCTCCCAGGAATGGTC |
| | | sequence | CCCAAACTCCTCATTTATGCCAATAATAAGCGACCCTCAGGGAT |
| | | | TCCTGACCGATTCTCTGGCTCCAGGTCTGGCACGTCAGCCACCC |
| | | | TGGGCATCACCGGACTCCAGACTGGGGACGAGGCCGATTATTAC |
| | | | TGCGGAACTTGGGATAGTAGTCGGAGTGCTGTGCTTTTCGGCGG |
| | | | AGGGACCAAGATGACCGTCCTA |
| 1210 | YANG-2203j | VL domain | QSVLTQPPSVSAAPGQKVTISCSGSSSNIGNNFVSWYQQLPGMV |
| | | amino | PKLLIYANNKRPSGIPDRFSGSRSGTSATLGITGLQTGDEADYY |
| | | acid | CGTWDSSRSAVLFGGGTKMTVL |
| | | sequence | |
| 1211 | YANG-2203j | LCDR1 | SSNIGNNE |
| 1212 | YANG-2203j | LCDR2 | ANN |
| 1213 | YANG-2203j | LCDR3 | GTWDSSRSAVL |
| 1214 | YANG-2203k | VH domain | CAGGTGCAACTGGTGCAGTCTGGGGCTGAGGTGAAGCAGCCTGG |
| | | nucleic | GGCCTCAGTGAAGGTTTCCTGCAGGGCATCTGGATACGCCCTCA |
| | | acid | CCAGACTCTATGTTCACTGGGTGCGGCAGGCCCCTGGACAAGGG |
| | | sequence | CTTGAGTGGATGGGCATAATCAAACCCAGTGGTGGTGACACAAT |
| | | | CTGCGCTCAGAATCTCCAGGGCAGAGTCACCCTGACCAGGGACA |
| | | | CGTCCACGAACACATTCTACGTGGAACTGAACAGCCTGAGATCT |
| | | | GAGGACACGGCCCTATATTACTGTGCGATCAGTGGGAACACTAG |
| | | | GGCTTTTGAAATCTGGGGCCAAGGGACAATGGTCACCGTCTCTT |
| | | | TA |
| 1215 | YANG-2203k | VH domain | QVQLVQSGAEVKQPGASVKVSCRASGYALTRLYVHWVRQAPGQG |
| | | amino | LEWMGIIKPSGGDTICAQNLQGRVTLTRDTSTNTFYVELNSLRS |
| | | acid | EDTALYYCAISGNTRAFEIWGQGTMVTVSL |
| | | sequence | |
| 1216 | YANG-2203k | HCDR1 | GYALTRLY |
| 1217 | YANG-2203k | HCDR2 | IKPSGGDT |
| 1218 | YANG-2203k | HCDR3 | AISGNTRAFEI |
| 1219 | YANG-2203k | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTTTGTATCCTGGTACCAGCAGCTCCCAGGAATGGTC |
| | | sequence | CCCAAACTCCTCATTTATGCCAATAATAAGCGACCCTCAGGGAT |
| | | | TCCTGACCGATTCTCTGGCTCCAGGTCTGGCACGTCAGCCACCC |
| | | | TGGGCATCACCGGACTCCAGACTGGGGACGAGGCCGATTATTAC |
| | | | TGCGGAACTTGGGATAGTAGTCGGAGTGCGGTGCTTTTCGGCGG |
| | | | AGGGACCAAGATGACCGTCCTA |
| 1220 | YANG-2203k | VL domain | QSVLTQPPSVSAAPGQKVTISCSGSSSNIGNNFVSWYQQLPGMV |
| | | amino | PKLLIYANNKRPSGIPDRFSGSRSGTSATLGITGLQTGDEADYY |
| | | acid | CGTWDSSRSAVLFGGGTKMTVL |
| | | sequence | |
| 1221 | YANG-2203k | LCDR1 | SSNIGNNF |
| 1222 | YANG-2203k | LCDR2 | ANN |
| 1223 | YANG-2203k | LCDR3 | GTWDSSRSAVL |
| 1224 | YANG-2204 | VH domain | CAGATGCAGCTGGTACAGTCTGGGGCTGAGGTGAAGAAGCCTGG |
| | | nucleic | GGCCTCAGTGAAGTTTTCCTGCAAGACATCTGGATACACCTTCA |
| | | acid | CCAACTACTATATGGTCTGGGTGCGACAGGCCCCTGGACAAGGA |
| | | sequence | CTTGAGTGGATGGGCATAATCAAACCTAGTGGTGGTAACACAAT |
| | | | CTATGCACAGAAGTTCCAGGGCAGAGTCACCATGACCAGGGACA |
| | | | CGTCCACGAACACAGTCTACATGGACCTGACCAGTCTGAGATCT |
| | | | GAAGACACGGCCGTATATTATTGTGCGAGAACTGGGGATAGGGC |
| | | | TTTTGATGTCTGGGGCCAAGGGACAATGGTCACCGTCTCTTCA |
| 1225 | YANG-2204 | VH domain | QMQLVQSGAEVKKPGASVKFSCKTSGYTFTNYYMVWVRQAPGQG |
| | | amino | LEWMGIIKPSGGNTIYAQKFQGRVTMTRDTSTNTVYMDLTSLRS |
| | | acid | EDTAVYYCARTGDRAFDVWGQGTMVTVSS |
| | | sequence | |
| 1226 | YANG-2204 | HCDR1 | GYTFTNYY |
| 1227 | YANG-2204 | HCDR2 | IKPSGGNT |
| 1228 | YANG-2204 | HCDR3 | ARTGDRAFDV |
| 1229 | YANG-2204 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTATGTCTCCTGGTACCAGCAGCTCCCAGGAACAGCC |
| | | sequence | CCCAAACTCCTCATTTATGACAATAATAAGCGCCCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAACTGACCGTCCTA |
| 1230 | YANG-2204 | VL domain | QSVLTQPPSVSAAPGQKVTISCSGSSSNIGNNYVSWYQQLPGTA |
| | | amino | PKLLIYDNNKRPSGIPDRFSGSKSGTSATLGITGLQTGDEADYY |
| | | acid | CGTWDSSLGAGVFGGGTKLTVL |
| | | sequence | |
| 1231 | YANG-2204 | LCDR1 | SSNIGNNY |
| 1232 | YANG-2204 | LCDR2 | DNN |
| 1233 | YANG-2204 | LCDR3 | GTWDSSLGAGV |
| 1234 | YANG-2205 | VH domain | CAGATTCAGTTGGTGCAGTCTGGGGCTGAGGTGAAGAAGCCTGG |
| | | nucleic | GGCCTCAGTGAAGTTTTCCTGCAAGACATCTGGATACACCTTCA |
| | | acid | CCAGCTACTATATGGTCTGGGTGCGACAGGCCCCTGGACAAGGA |
| | | sequence | CTTGAGTGGATGGGCATAATCAAACCTAGTGGTGGTAACACAAT |
| | | | CTACGCACAGAAGTTCCAGGGCAGAGTCACCATGACCAGGGACA |
| | | | CGTCCACGAACACAGTCTACATGGACCTGAGCAGTCTGAGATCT |
| | | | GAGGACACGGCCGTGTATTATTGTGCGAGAACTGGGGATAGGGC |
| | | | TTTTGATGTCTGGGGCCACGGGACAATGATCACCGTCTCTTCA |
| 1235 | YANG-2205 | VH domain | QIQLVQSGAEVKKPGASVKFSCKTSGYTFTSYYMVWVRQAPGQG |
| | | amino | LEWMGIIKPSGGNTIYAQKFQGRVTMTRDTSINTVYMDLSSLRS |
| | | acid | EDTAVYYCARTGDRAFDVWGHGTMITVSS |
| | | sequence | |
| 1236 | YANG-2205 | HCDR1 | GYTFTSYY |
| 1237 | YANG-2205 | HCDR2 | IKPSGGNT |
| 1238 | YANG-2205 | HCDR3 | ARTGDRAFDV |
| 1239 | YANG-2205 | VL domain | CAGTCTGTATTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTTTGTATCCTGGTACCAACAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGCTGACCGTCCTA |
| 1240 | YANG-2205 | VL domain | QSVLTQPPSVSAAPGQKVTISCSGSSSNIGNNFVSWYQQFPGTA |
| | | amino | PKLLIYDNNKRPSGIPDRFSGSKSGTSATLGITGLQTGDEADYY |
| | | acid | CGTWDSSLGAGVFGGGTKLTVL |
| | | sequence | |
| 1241 | YANG-2205 | LCDR1 | SSNIGNNF |
| 1242 | YANG-2205 | LCDR2 | DNN |
| 1243 | YANG-2205 | LCDR3 | GTWDSSLGAGV |
| 1244 | YANG-2206 | VH domain | CAGATTCAGTTGGTGCAGTCTGGGGCTGAGGTGAAGAAGCCTGG |
| | | nucleic | GGCCTCAGTGAAACTTTCCTGCAAGACATCTGGATACACCTTCA |
| | | acid | CCAGCTACTATATGGTCTGGGTGCGACAGGCCCCTGGACAAGGA |
| | | sequence | CTTGAGTGGATGGGCATAATCAAACCTAGTGGTGGTAACACAAT |
| | | | CTTCGCACAGAAGTTCCAGGGCAGAGTCACCATGACCAGGGACA |
| | | | CGTCCACGAACACAGTCTACATGGACCTGAACAGTCTGAGATCT |
| | | | GAGGACACGGCCGTCTATTATTGTGCGAGAACTGGAGATAGGGC |
| | | | TTTTGATGTCTGGGGCCACGGGACAATGGTCACCGTGTCTTCA |
| 1245 | YANG-2206 | VH domain | QIQLVQSGAEVKKPGASVKLSCKTSGYTFTSYYMVWVRQAPGQG |
| | | amino | LEWMGIIKPSGGNTIFAQKFQGRVTMTRDTSTNTVYMDLNSLRS |
| | | acid | EDTAVYYCARTGDRAFDVWGHGTMVTVSS |
| | | sequence | |
| 1246 | YANG-2206 | HCDR1 | GYTFTSYY |
| 1247 | YANG-2206 | HCDR2 | IKPSGGNT |
| 1248 | YANG-2206 | HCDR3 | ARTGDRAFDV |
| 1249 | YANG-2206 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTATGTATCCTGGTACCAGCAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGGTGACCGTCCTA |
| 1250 | YANG-2206 | VL domain | QSVLTQPPSVSAAPGQKVTISCSGSSSNIGNNYVSWYQQFPGTA |
| | | amino | PKLLIYDNNKRPSGIPDRFSGSKSGTSATLGITGLQTGDEADYY |
| | | acid | CGTWDSSLGAGVFGGGTKVTVL |
| | | sequence | |
| 1251 | YANG-2206 | LCDR1 | SSNIGNNY |
| 1252 | YANG-2206 | LCDR2 | DNN |
| 1253 | YANG-2206 | LCDR3 | GTWDSSLGAGV |
| 1254 | YANG-2207 | VH domain | CAGATGCAATTGGTGCAATCTGGGGCTGAGGTGAAGAAGCCTGG |
| | | nucleic | GGCCTCAGTGAAGATTTCCTGCAAGACATCTGGATACACCTTCA |
| | | acid | CCAACTACTATATGGTCTGGGTGCGACAGGCCCCTGGACAAGGA |
| | | sequence | CTTGAGTGGATGGGCATAATCAAACCTAGTGGTGGTAACACGAT |
| | | | CTACGCACAGAAGTTCCAGGGCAGAGTCACCATGACCAGGGACA |
| | | | CGTCCACGAACACAGTCTATTTGGACCTGAGCAGTCTGAGATCT |
| | | | GAGGACACGGCCGTCTATTATTGTGCGAGAACTGGGAATAGGGC |
| | | | TTTTGATGTCTGGGGCCACGGGACAATGGTCACCGTCTCTTCA |
| 1255 | YANG-2207 | VH domain | QMQLVQSGAEVKKPGASVKISCKTSGYTFTNYYMVWVRQAPGQG |
| | | amino | LEWMGIIKPSGGNTIYAQKFQGRVTMTRDTSTNTVYLDLSSLRS |
| | | acid | EDTAVYYCARTGNRAFDVWGHGTMVTVSS |
| | | sequence | |
| 1256 | YANG-2207 | HCDR1 | GYTFTNYY |
| 1257 | YANG-2207 | HCDR2 | IKPSGGNT |
| 1258 | YANG-2207 | HCDR3 | ARTGNRAFDV |
| 1259 | YANG-2207 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTTTGTTTCCTGGTACCAGCAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGCTGACCGTCCTA |
| 1260 | YANG-2207 | VL domain | QSVLTQPPSVSAAPGQKVTISCSGSSSNIGNNFVSWYQQFPGTA |
| | | amino | PKLLIYDNNKRPSGIPDRFSGSKSGTSATLGITGLQTGDEADYY |
| | | acid | CGTWDSSLGAGVFGGGTKLTVL |
| | | sequence | |
| 1261 | YANG-2207 | LCDR1 | SSNIGNNF |
| 1262 | YANG-2207 | LCDR2 | DNN |
| 1263 | YANG-2207 | LCDR3 | GTWDSSLGAGV |
| 1264 | YANG-2208 | VH domain | CAGATACAGCTGGTGCAGTCTGGGGCTGAGGTGAGGAAGCCTGG |
| | | nucleic | GGCCTCAGTGAGGTTTTCCTGCAAGACATCTGGATACATTTTCA |
| | | acid | CCAACTACTATATGGTCTGGGTGCGACAGGCCCCTGGACAAGGA |
| | | sequence | CTTGAGTGGATGGGCATAATCAAACCTAGTGGAGGTAACACAAT |
| | | | CTACGCACAGAAGTTCCAGGGCAGAGTCACCATGACCAGGGACA |
| | | | CGTCCACTAACACAGTCTACATGGACCTGAATAATCTGAGATCT |
| | | | GAGGACACGGCCGTGTATTTTTGTGCGAGAACTGGTGATAGGGC |
| | | | TTTTGATGTCTGGGGCCAAGGGACAATGGTCACCGTCTCTTCA |
| 1265 | YANG-2208 | VH domain | QIQLVQSGAEVRKPGASVRFSCKTSGYIFTNYYMVWVRQAPGQG |
| | | amino | LEWMGIIKPSGGNTIYAQKFQGRVTMTRDTSTNTVYMDLNNLRS |
| | | acid | EDTAVYFCARTGDRAFDVWGQGTMVTVSS |
| | | sequence | |
| 1266 | YANG-2208 | HCDR1 | GYIFTNYY |
| 1267 | YANG-2208 | HCDR2 | IKPSGGNT |
| 1268 | YANG-2208 | HCDR3 | ARTGDRAFDV |
| 1269 | YANG-2208 | VL domain | CAGTCTGTGTTGACGCAGCCGCCCTCAATGTCTGCGGCCCCAGG |
| | | nucleic | ACAGAAGGTCACCATCTCCTGCTCTGGAAACAGCTCCAACATTG |
| | | acid | GGAATAATTTTGTATCCTGGTACCGGCAGTTCCCCGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGGACATGGGATAGCAGCCTGAGTGCTGGGGTGTTCGGCGG |
| | | | AGGGACCAAGCTGACCGTCCTA |
| 1270 | YANG-2208 | VL domain | QSVLTQPPSMSAAPGQKVTISCSGNSSNIGNNFVSWYRQFPGTA |
| | | amino | PKLLIYDNNKRPSGIPDRFSGSKSGTSATLGITGLQTGDEADYY |
| | | acid | CGTWDSSLSAGVFGGGTKLTVL |
| | | sequence | |
| 1271 | YANG-2208 | LCDR1 | SSNIGNNE |
| 1272 | YANG-2208 | LCDR2 | DNN |
| 1273 | YANG-2208 | LCDR3 | GTWDSSLSAGV |
| 1274 | YANG-2209 | VH domain | CAGATGCAGTTGGTGCAGTCTGGGGCTGAGGTGAAGAAGCCTGG |
| | | nucleic | GGCCTCAGTGAAGTTTTCCTGCAAGACATCTGGATACACCTTCA |
| | | acid | CCAACTTCTATATGGTCTGGGTGCGACAGGCCCCTGGACAAGGA |
| | | sequence | CTTGAGTGGATGGGCATAATCAAACCTAGTGGTGGTGACACAAT |
| | | | CTACGCACAGAAGTTCCAGGGCAGAGTCACCATGACCAGGGACA |
| | | | CGTCCACGAACACAGTCTACATGGACCTGAACAGTCTGACATCT |
| | | | GAGGACACGGCCGTATATTATTGTGCGAGAATTGGGAATAGGGC |
| | | | TTTTGATGTCTGGGGCCACGGGACAATGGTCACCGTCTCTTCA |
| 1275 | YANG-2209 | VH domain | QMQLVQSGAEVKKPGASVKFSCKTSGYTFTNFYMVWVRQAPGQG |
| | | amino | LEWMGIIKPSGGDTIYAQKFQGRVTMTRDTSTNTVYMDLNSLTS |
| | | acid | EDTAVYYCARIGNRAFDVWGHGTMVTVSS |
| | | sequence | |
| 1276 | YANG-2209 | HCDR1 | GYTFTNFY |
| 1277 | YANG-2209 | HCDR2 | IKPSGGDT |
| 1278 | YANG-2209 | HCDR3 | ARIGNRAFDV |
| 1279 | YANG-2209 | VL domain | CAGTCTGTGTTGACGCAGCCGCCCTCAGTGTCTGCGGCCCCAGG |
| | | nucleic | ACAGAAAGTCACCATCTCCTGCTCTGGAAGCAGCTCCAATATTG |
| | | acid | GGAATAATTTTGTATCCTGGTACCAGCAGTTTTCAGGAACAGCC |
| | | sequence | CCCAAACTCCTCATTTATGACAATTCTAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAACTGACCGTCCTA |
| 1280 | YANG-2209 | VL domain | QSVLTQPPSVSAAPGQKVTISCSGSSSNIGNNFVSWYQQFSGTA |
| | | amino | PKLLIYDNSKRPSGIPDRFSGSKSGTSATLGITGLQTGDEADYY |
| | | acid | CGTWDSSLGAGVFGGGTKLTVL |
| | | sequence | |
| 1281 | YANG-2209 | LCDR1 | SSNIGNNF |
| 1282 | YANG-2209 | LCDR2 | DNS |
| 1283 | YANG-2209 | LCDR3 | GTWDSSLGAGV |
| 1284 | YANG-2210 | VH domain | CAGATTCAGTTGGTGCAGTCTGGGGCTGAGGTGAAGAAGCCTGG |
| | | nucleic | GGCCTCAGTGAAGTTTTCCTGCAAGACATCTGGATACACCTTCA |
| | | acid | CCAGCTACTATATGGTCTGGGTGCGACAGGCCCCTGGACAAGGA |
| | | sequence | CTTGAGTGGATGGGCATAATCAAACCTAGTGGTGGTAACACAAT |
| | | | CTACGCACAGAAGTTCCAGGGCAGAGTCACCATGACCAGGGACA |
| | | | CGTCCACGAACACAGTCTACATGGACCTGAACAGTCTGAAATCT |
| | | | GAGGACACGGCCGTCTATTATTGTGCGAGAACTGGAGATTGGGC |
| | | | TTTTGATGTCTGGGGCCACGGGACAATGGTCACCGTGTCTTCA |
| 1285 | YANG-2210 | VH domain | QIQLVQSGAEVKKPGASVKFSCKTSGYTFTSYYMVWVRQAPGQG |
| | | amino | LEWMGIIKPSGGNTIYAQKFQGRVTMTRDTSTNTVYMDLNSLKS |
| | | acid | EDTAVYYCARTGDWAFDVWGHGTMVTVSS |
| | | sequence | |
| 1286 | YANG-2210 | HCDR1 | GYTFTSYY |
| 1287 | YANG-2210 | HCDR2 | IKPSGGNT |
| 1288 | YANG-2210 | HCDR3 | ARTGDWAFDV |
| 1289 | YANG-2210 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTATGTATCCTGGTACCAGCAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAACTGACCGTCCTA |
| 1290 | YANG-2210 | VL domain | QSVLTQPPSVSAAPGQKVTISCSGSSSNIGNNYVSWYQQFPGTA |
| | | amino | PKLLIYDNNKRPSGIPDRFSGSKSGTSATLGITGLQTGDEADYY |
| | | acid | CGTWDSSLGAGVFGGGTKLTVL |
| | | sequence | |
| 1291 | YANG-2210 | LCDR1 | SSNIGNNY |
| 1292 | YANG-2210 | LCDR2 | DNN |
| 1293 | YANG-2210 | LCDR3 | GTWDSSLGAGV |
| 1294 | YANG-2211 | VH domain | CAGATACAACTGGTGCAGTCTGGGGCTGAGGTGAAGAAGCCTGG |
| | | nucleic | GGCCTCTGTGAGGTTTTCCTGCAAGACATCTGGATACACCTTCA |
| | | acid | CCAACTACTATATGGTCTGGGTGCGACAGGCCCCTGGACAAGGA |
| | | sequence | CTTGAGTGGATGGGCATAATTAAACCTAGTGGTGGTAACACAAT |
| | | | CTACGCACAGAACTTCCAGGGCAGAGTCACCATGACCAGGGACA |
| | | | CGTCCACGAACACAGTCTACATGGACCTGAGGACTCTGAGATCT |
| | | | GAGGACACGGCCGTTTATTATTGTGCGAGAACTGGTGATAGGGC |
| | | | TTTTGATGTCTGGGGCCAAGGGACAATGGTCACCGTCTCTTCA |
| 1295 | YANG-2211 | VH domain | QIQLVQSGAEVKKPGASVRFSCKTSGYTFTNYYMVWVRQAPGQG |
| | | amino | LEWMGIIKPSGGNTIYAQNFQGRVTMTRDTSTNTVYMDLRTLRS |
| | | acid | EDTAVYYCARTGDRAFDVWGQGTMVTVSS |
| | | sequence | |
| 1296 | YANG-2211 | HCDR1 | GYTFTNYY |
| 1297 | YANG-2211 | HCDR2 | IKPSGGNT |
| 1298 | YANG-2211 | HCDR3 | ARTGDRAFDV |
| 1299 | YANG-2211 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTTTGTATCCTGGTACCAGCAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGCTGACCGTCCTA |
| 1300 | YANG-2211 | VL domain | QSVLTQPPSVSAAPGQKVTISCSGSSSNIGNNFVSWYQQFPGTA |
| | | amino | PKLLIYDNNKRPSGIPDRFSGSKSGTSATLGITGLQTGDEADYY |
| | | acid | CGTWDSSLGAGVFGGGTKLTVL |
| | | sequence | |
| 1301 | YANG-2211 | LCDR1 | SSNIGNNF |
| 1302 | YANG-2211 | LCDR2 | DNN |
| 1303 | YANG-2211 | LCDR3 | GTWDSSLGAGV |
| 1304 | YANG-2212 | VH domain | CAGATGCAGTTGGTGCAGTCTGGGGCTGAGGTGAAGAAGCCTGG |
| | | nucleic | GGCCTCAGTGAAAATTTCCTGCAAGACATCTGGATACACCTTCA |
| | | acid | TCAACTACTATATGGTCTGGGTGCGACAGGCCCCTGGACAAGGA |
| | | sequence | CTTGAGTGGATGGGCATAATCAAACCTAGTGGTGGTAATACGAT |
| | | | CTACGCACAGAAGTTCCAGGGCAGAGTCACCATGACCAGGGACA |
| | | | CGTCCACGAGCACAGTCTATATGGACCTGAGCAGTCTGAGATCT |
| | | | GAGGACACGGCCGTGTATTATTGTGCGAGAACTGGGGATAGGGC |
| | | | TTTTGATGTCTGGGGCCACGGGACAATGGTCACCGTCTCTTCA |
| 1305 | YANG-2212 | VH domain | QMQLVQSGAEVKKPGASVKISCKTSGYTFINYYMVWVRQAPGQG |
| | | amino | LEWMGIIKPSGGNTIYAQKFQGRVTMTRDTSTSTVYMDLSSLRS |
| | | acid | EDTAVYYCARTGDRAFDVWGHGTMVTVSS |
| | | sequence | |
| 1306 | YANG-2212 | HCDR1 | GYTFINYY |
| 1307 | YANG-2212 | HCDR2 | IKPSGGNT |
| 1308 | YANG-2212 | HCDR3 | ARTGDRAFDV |
| 1309 | YANG-2212 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTATGTTTCCTGGTACCAGCAGTTCCCAGGAACAGCC |
| | | sequence | CCCAAACTCCTCATTTATGACAATCATAAGCGACCCTCAGGGAT |
| | | | TCCTGACCGATTCTCTGCCTCCAAGTCTGGCACGTCAGCCACCC |
| | | | TGGGCATCACCGGACTCCTGACTGGGGACGAGGCCGATTATTAC |
| | | | TGCGGAACATGGGATAGCGGCCTGGGTGCTGGGGTGTTCGGCGG |
| | | | AGGGACCAAGCTGACCGTCCTA |
| 1310 | YANG-2212 | VL domain | QSVLTQPPSVSAAPGQKVTISCSGSSSNIGNNYVSWYQQFPGTA |
| | | amino | PKLLIYDNHKRPSGIPDRFSASKSGTSATLGITGLLTGDEADYY |
| | | acid | CGTWDSGLGAGVFGGGTKLTVL |
| | | sequence | |
| 1311 | YANG-2212 | LCDR1 | SSNIGNNY |
| 1312 | YANG-2212 | LCDR2 | DNH |
| 1313 | YANG-2212 | LCDR3 | GTWDSGLGAGV |
| 1314 | YANG-2213 | VH domain | CAGATACAACTGGTGCAGTCTGGGGCTGAGGTGAAGAAGCCTGG |
| | | nucleic | GGCCTCAGTGAAGTTTTCCTGCAAGACATCTGGATACACCTTTA |
| | | acid | CCAACTACTATATGGTCTGGGTGCGACAGGCCCCTGGACAAGGA |
| | | sequence | CTTGAGTGGATGGGCATAATCAAACCTAGAGGTGGTGACACAAT |
| | | | CTACGCACAGAACTTCCAGGGCAGAGTCACCATGACCAGGGACA |
| | | | CGTCCACGAACACAGTCTATATGGACCTGAGGAGTCTGAGATCT |
| | | | GAGGACACGGCCGTGTATTATTGTGCGAGAACTGGTGATAGGGC |
| | | | TTTTGATGTCTGGGGCCAAGGGACAATGGTCACCGTCTCTTCA |
| 1315 | YANG-2213 | VH domain | QIQLVQSGAEVKKPGASVKFSCKTSGYTFTNYYMVWVRQAPGQG |
| | | amino | LEWMGIIKPRGGDTIYAQNFQGRVTMTRDTSTNTVYMDLRSLRS |
| | | acid | EDTAVYYCARTGDRAFDVWGQGTMVTVSS |
| | | sequence | |
| 1316 | YANG-2213 | HCDR1 | GYTFTNYY |
| 1317 | YANG-2213 | HCDR2 | IKPRGGDT |
| 1318 | YANG-2213 | HCDR3 | ARTGDRAFDV |
| 1319 | YANG-2213 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTTTGTATCCTGGTACGAGCAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGCTGACCGTCCTA |
| 1320 | YANG-2213 | VL domain | QSVLTQPPSVSAAPGQKVTISCSGSSSNIGNNFVSWYEQFPGTA |
| | | amino | PKLLIYDNNKRPSGIPDRFSGSKSGTSATLGITGLQTGDEADYY |
| | | acid | CGTWDSSLGAGVFGGGTKLTVL |
| | | sequence | |
| 1321 | YANG-2213 | LCDR1 | SSNIGNNF |
| 1322 | YANG-2213 | LCDR2 | DNN |
| 1323 | YANG-2213 | LCDR3 | GTWDSSLGAGV |
| 1324 | YANG-2214 | VH domain | CAGATACAACTGGTGCAGTCTGGGGCTGAGGTGAAGAAGCCTGG |
| | | nucleic | GGCCTCAGTGAGGTTTTCCTGCAAGACATCTGGATACACCTTCA |
| | | acid | CCAACTACTATATGGTCTGGGTGCGACAGGCCCCTGGACAAGGA |
| | | sequence | CTTGAGTGGATGGGCATCATCAAACCTAGTGGTGGTAACACCAT |
| | | | CTACGCACAGAACTTCCAGGGCAGAGTCTCCATGACCAGGGACA |
| | | | CGTCCACGAGCACAGTCTACATGGACCTGAGGAGTCTGACATCT |
| | | | GAGGACACGGCCGTGTATTATTGTGCGAGAACTGGTGATAGGGC |
| | | | TTTTGATGTCTGGGGCCAAGGGACAATGGTCACCGTCTCTTCA |
| 1325 | YANG-2214 | VH domain | QIQLVQSGAEVKKPGASVRFSCKTSGYTFTNYYMVWVRQAPGQG |
| | | amino | LEWMGIIKPSGGNTIYAQNFQGRVSMTRDTSTSTVYMDLRSLTS |
| | | acid | EDTAVYYCARTGDRAFDVWGQGTMVTVSS |
| | | sequence | |
| 1326 | YANG-2214 | HCDR1 | GYTFTNYY |
| 1327 | YANG-2214 | HCDR2 | IKPSGGNT |
| 1328 | YANG-2214 | HCDR3 | ARTGDRAFDV |
| 1329 | YANG-2214 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTTTGTATCCTGGTACCAGCAGTTTCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGCTGACCGTCCTA |
| 1330 | YANG-2214 | VL domain | QSVLTQPPSVSAAPGQKVTISCSGSSSNIGNNFVSWYQQFPGTA |
| | | amino | PKLLIYDNNKRPSGIPDRFSGSKSGTSATLGITGLQTGDEADYY |
| | | acid | CGTWDSSLGAGVFGGGTKLTVL |
| | | sequence | |
| 1331 | YANG-2214 | LCDR1 | SSNIGNNF |
| 1332 | YANG-2214 | LCDR2 | DNN |
| 1333 | YANG-2214 | LCDR3 | GTWDSSLGAGV |
| 1334 | YANG-2215 | VH domain | CAGATACAGTTGGTGCAGTCTGGGCCTGAGGTGAAGAAGCCTGG |
| | | nucleic | GGCCTCAGTGAAGATTTCCTGCAAGACATCTGGATACACCTTCA |
| | | acid | CCAACTACTATATGGTCTGGGTGCGACAGGCCCCTGGACAAGGA |
| | | sequence | CTTGAGTGGATGGGCATAATCAAACCTAGTGGTGGTAACACGAT |
| | | | CTACGCACAGAAGTTCCAGGGCAGAGTCACCATGACCAGGGACA |
| | | | CGTCCACGAACACAGTCTATTTGGACCTGAGCAGTCTGAGATCT |
| | | | GAGGACACGGCCGTGTATTATTGTGCGAGAACTGGGGATAGGGC |
| | | | TTTTGATGTCTGGGGCCACGGGACAATGGTCACCGTCTCTTCA |
| 1335 | YANG-2215 | VH domain | QIQLVQSGPEVKKPGASVKISCKTSGYTFTNYYMVWVRQAPGQG |
| | | amino | LEWMGIIKPSGGNTIYAQKFQGRVTMTRDTSTNTVYLDLSSLRS |
| | | acid | EDTAVYYCARTGDRAFDVWGHGTMVTVSS |
| | | sequence | |
| 1336 | YANG-2215 | HCDR1 | GYTFTNYY |
| 1337 | YANG-2215 | HCDR2 | IKPSGGNT |
| 1338 | YANG-2215 | HCDR3 | ARTGDRAFDV |
| 1339 | YANG-2215 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTATGTTTCCTGGTACCAGAAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGCTGACCGTCCTA |
| 1340 | YANG-2215 | VL domain | QSVLTQPPSVSAAPGQKVTISCSGSSSNIGNNYVSWYQKFPGTA |
| | | amino | PKLLIYDNNKRPSGIPDRFSGSKSGTSATLGITGLQTGDEADYY |
| | | acid | CGTWDSSLGAGVFGGGTKLTVL |
| | | sequence | |
| 1341 | YANG-2215 | LCDR1 | SSNIGNNY |
| 1342 | YANG-2215 | LCDR2 | DNN |
| 1343 | YANG-2215 | LCDR3 | GTWDSSLGAGV |
| 1344 | YANG-2216 | VH domain | CAGATTCAGTTGGTGCAGTCTGGGGCTGAGGTGAAGAAGCCTGG |
| | | nucleic | GGCCTCAGTGAAGCTTTCCTGCAAGACATCTGGATACACCTTCA |
| | | acid | CCAGCTACTACATGGTCTGGGTGCGACAGGCCCCTGGACAAGGA |
| | | sequence | CTTGAGTGGATGGGCATAATCAAACCTAGTGGTGGTAACACACT |
| | | | CTTCGAACAGAAGTTTCAGGGTAGAGTCATCATGACCAGGGACA |
| | | | CGTCCACGAACACAGTCTACATGGACCTGAACAGTCTGAGATCT |
| | | | GAGGACACGGCCGTCTATTATTGTGCGAGAACTGGAGATAGGGC |
| | | | TTTTGATGTCTGGGGCCACGGGACAATGGTCACCGTGTCTTCA |
| 1345 | YANG-2216 | VH domain | QIQLVQSGAEVKKPGASVKLSCKTSGYTFTSYYMVWVRQAPGQG |
| | | amino | LEWMGIIKPSGGNTLFEQKFQGRVIMTRDTSTNTVYMDLNSLRS |
| | | acid | EDTAVYYCARTGDRAFDVWGHGTMVTVSS |
| | | sequence | |
| 1346 | YANG-2216 | HCDR1 | GYTFTSYY |
| 1347 | YANG-2216 | HCDR2 | IKPSGGNT |
| 1348 | YANG-2216 | HCDR3 | ARTGDRAFDV |
| 1349 | YANG-2216 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTATGTATCCTGGTACCAGCAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGCTGACCGTCCTA |
| 1350 | YANG-2216 | VL domain | QSVLTQPPSVSAAPGQKVTISCSGSSSNIGNNYVSWYQQFPGTA |
| | | amino | PKLLIYDNNKRPSGIPDRFSGSKSGTSATLGITGLQTGDEADYY |
| | | acid | CGTWDSSLGAGVFGGGTKLTVL |
| | | sequence | |
| 1351 | YANG-2216 | LCDR1 | SSNIGNNY |
| 1352 | YANG-2216 | LCDR2 | DNN |
| 1353 | YANG-2216 | LCDR3 | GTWDSSLGAGV |
| 1354 | YANG-2217 | VH domain | CAGATGCAATTGGTGCAGTCTGGGGCTGAGGTGAAGAAGCCTGG |
| | | nucleic | GGCCTCAGTGAAGATTTCCTGCAAGACATCTGGATACACCTTCA |
| | | acid | CCAACTACTATATGGTCTGGGTGCGACAGGCCCCTGGACAAGGA |
| | | sequence | CTTGAGTGGATGGGCATAATCAAACCTAGTGGTGGTAACACGAT |
| | | | CTACGCACAGAAGTTCCAGGGCAGAGTCACCGTGACCAGGGACA |
| | | | CGTCCACGAACACAGTCTATTTGGACCTGAGCAGTCTTACATCT |
| | | | GAAGACACGGCCGTATATTATTGTGCGAGAACTGGGGATAGGGC |
| | | | TTTTGATGTCTGGGGCCATGGGACACTGGTCACCGTCTCTTCA |
| 1355 | YANG-2217 | VH domain | QMQLVQSGAEVKKPGASVKISCKTSGYTFTNYYMVWVRQAPGQG |
| | | amino | LEWMGIIKPSGGNTIYAQKFQGRVTVTRDTSTNTVYLDLSSLTS |
| | | acid | EDTAVYYCARTGDRAFDVWGHGTLVTVSS |
| | | sequence | |
| 1356 | YANG-2217 | HCDR1 | GYTFTNYY |
| 1357 | YANG-2217 | HCDR2 | IKPSGGNT |
| 1358 | YANG-2217 | HCDR3 | ARTGDRAFDV |
| 1359 | YANG-2217 | VL domain | CAGTCTGTGTTGACGCAGCCGCCCTCAGTGTCTGCGGCCCCAGG |
| | | nucleic | ACAGAAGGTCACCATCTCCTGCTCTGGAAGAAGTTCCAACATTG |
| | | acid | GGAATAATTATGTTTCCTGGTACCAGCAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAACTGACCGTCCTA |
| 1360 | YANG-2217 | VL domain | QSVLTQPPSVSAAPGQKVTISCSGRSSNIGNNYVSWYQQFPGTA |
| | | amino | PKLLIYDNNKRPSGIPDRFSGSKSGTSATLGITGLQTGDEADYY |
| | | acid | CGTWDSSLGAGVFGGGTKLTVL |
| | | sequence | |
| 1361 | YANG-2217 | LCDR1 | SSNIGNNY |
| 1362 | YANG-2217 | LCDR2 | DNN |
| 1363 | YANG-2217 | LCDR3 | GTWDSSLGAGV |
| 1364 | YANG-2218 | VH domain | CAGATTCAGTTGGTGCAGTCTGGGGCTGAGGTGAAGAAGCCTGG |
| | | nucleic | GGCCTCAGTGAAGTTTTCCTGCAAGACATCTGGATACACCTTCA |
| | | acid | CCAGCTACTATATGGTCTGGGTGCGACAGGCCCCTGGACAAGGA |
| | | sequence | CTTGAGTGGATGGGCATAATCAAACCTAGTGGTGGTAACACAAT |
| | | | CTACGCACAGAAGTTCCAGGGCAGAGTCACCATGACCAGGGACA |
| | | | CGTCCACGAACACAGTCTACATGGACCTGAGCAGTCTGAGATCT |
| | | | GAGGACACGGCCGTGTATTATTGTGCGAGATCTGGGGATAGGGC |
| | | | TTTTGATGTCTGGGGCCACGGGACAATGATCACCGTCTCTTCA |
| 1365 | YANG-2218 | VH domain | QIQLVQSGAEVKKPGASVKFSCKTSGYTFTSYYMVWVRQAPGQG |
| | | amino | LEWMGIIKPSGGNTIYAQKFQGRVTMTRDTSTNTVYMDLSSLRS |
| | | acid | EDTAVYYCARSGDRAFDVWGHGTMITVSS |
| | | sequence | |
| 1366 | YANG-2218 | HCDR1 | GYTFTSYY |
| 1367 | YANG-2218 | HCDR2 | IKPSGGNT |
| 1368 | YANG-2218 | HCDR3 | ARSGDRAFDV |
| 1369 | YANG-2218 | VL domain | CAGTCTGTATTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTTTGTGTCCTGGTACCAACAGTTCCCAGGAACAGCC |
| | | sequence | CCCAAACTCCTCATTTATGACAATAATAAGCGACCCTCAGGGAT |
| | | | TCCTGACCGCTTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGCTGACCGTCCTA |
| 1370 | YANG-2218 | VL domain | QSVLTQPPSVSAAPGQKVTISCSGSSSNIGNNFVSWYQQFPGTA |
| | | amino | PKLLIYDNNKRPSGIPDRFSGSKSGTSATLGITGLQTGDEADYY |
| | | acid | CGTWDSSLGAGVFGGGTKLTVL |
| | | sequence | |
| 1371 | YANG-2218 | LCDR1 | SSNIGNNF |
| 1372 | YANG-2218 | LCDR2 | DNN |
| 1373 | YANG-2218 | LCDR3 | GTWDSSLGAGV |
| 1374 | YANG-2219 | VH domain | CAGATGCAATTGGTGCAATCTGGGGCTGAGGTGAAGAAGCCTGG |
| | | nucleic | GGCCTCAGTGAAGATTTCCTGCAAGACATCTGGATACACCTTCA |
| | | acid | CCAACTACTATATGGTCTGGGTGCGACAGGCCCCTGGACAAGGA |
| | | sequence | CTTGAGTGGATGGGCATAATCAAACCTAGTGGTGGTAACACGAT |
| | | | CTACGCACACAAGTTTCAGGGCAGAGTCACCATGACCAGGGACA |
| | | | CGTCCACGAACACAGTCTATTTGGACCTGAGCAGTCTGAGATCT |
| | | | GAGGACACGGCCGTCTATTATTGTGCCAGAACTGGGGATAGGGC |
| | | | TTTTGATGTCTGGGGCCACGGGACAATGGTCACCGTCTCTTCA |
| 1375 | YANG-2219 | VH domain | QMQLVQSGAEVKKPGASVKISCKTSGYTFTNYYMVWVRQAPGQG |
| | | amino | LEWMGIIKPSGGNTIYAHKFQGRVTMTRDTSTNTVYLDLSSLRS |
| | | acid | EDTAVYYCARTGDRAFDVWGHGTMVTVSS |
| | | sequence | |
| 1376 | YANG-2219 | HCDR1 | GYTFTNYY |
| 1377 | YANG-2219 | HCDR2 | IKPSGGNT |
| 1378 | YANG-2219 | HCDR3 | ARTGDRAFDV |
| 1379 | YANG-2219 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTTTGTTTCCTGGTACCAGCAGTTCCCAGGAACAGCC |
| | | sequence | CCCAAACTCCTCATTTATGACAATAATAAACGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGCTGACCGTCCTA |
| 1380 | YANG-2219 | VL domain | QSVLTQPPSVSAAPGQKVTISCSGSSSNIGNNFVSWYQQFPGTA |
| | | amino | PKLLIYDNNKRPSGIPDRFSGSKSGTSATLGITGLQTGDEADYY |
| | | acid | CGTWDSSLGAGVFGGGTKLTVL |
| | | sequence | |
| 1381 | YANG-2219 | LCDR1 | SSNIGNNE |
| 1382 | YANG-2219 | LCDR2 | DNN |
| 1383 | YANG-2219 | LCDR3 | GTWDSSLGAGV |
| 1384 | YANG-2220 | VH domain | CAGATACAACTGGTGCAGTCTGGGGCTGAGGTGAAGAAGCCTGG |
| | | nucleic | GGCCTCAGTGAAGTTTTCCTGCAAGACATCTGGATACACCTTCA |
| | | acid | CCAACTACTATATGGTCTGGGTGCGACAGGCCCCTGGACAAGGA |
| | | sequence | CTTGAGTGGATGGGCATAATCAAACCTAGTGGTGGTAACACAAT |
| | | | CTACGCACAGAACTTCCAGGACAGAGTCACCATGACCAGGGACA |
| | | | CGTCCACGAATACAGTCTACATGGACCTGAGGAGTCTGAGATCT |
| | | | GAGGACACGGCCGTGTATTATTGTGCGAGAACTGGTGATAGGGC |
| | | | TTTTGATGTCTGGGGCCATGGGACAATGGTCACCGTCTCTTCA |
| 1385 | YANG-2220 | VH domain | QIQLVQSGAEVKKPGASVKFSCKTSGYTFTNYYMVWVRQAPGQG |
| | | amino | LEWMGIIKPSGGNTIYAQNFQDRVTMTRDTSTNTVYMDLRSLRS |
| | | acid | EDTAVYYCARTGDRAFDVWGHGTMVTVSS |
| | | sequence | |
| 1386 | YANG-2220 | HCDR1 | GYTFTNYY |
| 1387 | YANG-2220 | HCDR2 | IKPSGGNT |
| 1388 | YANG-2220 | HCDR3 | ARTGDRAFDV |
| 1389 | YANG-2220 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GAAATAATTTTGTATCCTGGTACCAGCAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGCTGACCGTCCTA |
| 1390 | YANG-2220 | VL domain | QSVLTQPPSVSAAPGQKVTISCSGSSSNIGNNFVSWYQQFPGTA |
| | | amino | PKLLIYDNNKRPSGIPDRFSGSKSGTSATLGITGLQTGDEADYY |
| | | acid | CGTWDSSLGAGVFGGGTKLTVL |
| | | sequence | |
| 1391 | YANG-2220 | LCDR1 | SSNIGNNF |
| 1392 | YANG-2220 | LCDR2 | DNN |
| 1393 | YANG-2220 | LCDR3 | GTWDSSLGAGV |
| 1394 | YANG-2221 | VH domain | CAGATGCAATTGCTGCAGTCTGGGGCTGAGGTGAAGAAGCCTGG |
| | | nucleic | GGCCTCAGTGAAGATTTCCTGCAAGACATCTGGATACACCTTCA |
| | | acid | CCAACTACTATATGGTCTGGGTGCGACAGGCCCCTGGACAAGGA |
| | | sequence | CTTGAGTGGATGGGCATAATCAAACCTAGTGGTGGTAACACGAT |
| | | | CTACGCACAGAAGTTCCAGGGCAGAGTCACCATGACCAGGGACA |
| | | | CGTCCACGAACACAGTCTATTTGGACCTGAGCAGTCTGAGATCT |
| | | | GAGGACACGGCCGTGTATTATTGTGCGAGAACTGGGGATAGGGC |
| | | | TTTTGATGTCTGGGGCCACGGGACAATAGTCACCGTCTCTTCA |
| 1395 | YANG-2221 | VH domain | QMQLLQSGAEVKKPGASVKISCKTSGYTFTNYYMVWVRQAPGQG |
| | | amino | LEWMGIIKPSGGNTIYAQKFQGRVTMTRDTSTNTVYLDLSSLRS |
| | | acid | EDTAVYYCARTGDRAFDVWGHGTIVTVSS |
| | | sequence | |
| 1396 | YANG-2221 | HCDR1 | GYTFTNYY |
| 1397 | YANG-2221 | HCDR2 | IKPSGGNT |
| 1398 | YANG-2221 | HCDR3 | ARTGDRAFDV |
| 1399 | YANG-2221 | VL domain | CAGTCTGTGTTGACGCAGCCGCCCTCAGTGTCTGCGGCCCCAGG |
| | | nucleic | ACAGAAGGTCACCATCTCCTGCTCTGGAAACAGCTCCAACATTG |
| | | acid | GGAATAATTATGTTTCCTGGTATCAGCAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAT |
| | | | TGCGGAACATGGGATAGCAGCCTGGGTGCTGGGGTGTTCGGCGG |
| | | | AGGGACCAAGCTGACCGTCCTA |
| 1400 | YANG-2221 | VL domain | QSVLTQPPSVSAAPGQKVTISCSGNSSNIGNNYVSWYQQFPGTA |
| | | amino | PKLLIYDNNKRPSGIPDRFSGSKSGTSATLGITGLQTGDEADYY |
| | | acid | CGTWDSSLGAGVFGGGTKLTVL |
| | | sequence | |
| 1401 | YANG-2221 | LCDR1 | SSNIGNNY |
| 1402 | YANG-2221 | LCDR2 | DNN |
| 1403 | YANG-2221 | LCDR3 | GTWDSSLGAGV |
| 1404 | YANG-2222 | VH domain | CAGATACAGCTGGTGCAGTCTGGGGCTGAGGTGAAGAAGCCTGG |
| | | nucleic | GGCCTCAGTGAAGTTTTCCTGCAAGACATCTGGATACACCTTCA |
| | | acid | CCAACTACTATATGGTCTGGGTGCGACAGGCCCCTGGACAAGGA |
| | | sequence | CTTGAGTGGATGGGCATAATCAAACCTAGTGGTGGTAACACAAT |
| | | | CTACGCACAGAATTTCCAGGGCAGAGTCACCATGACCAGGGACA |
| | | | CGTCCACGAACACAGTCTACATGGACCTGAGGAGTCTGAGATCT |
| | | | GAGGACACGGCCGTGTATTATTGTGCGAGAACTGGTGATAGGGC |
| | | | TTTTGATGTCTGGGGCCAAGGGACAATGGTCACCGTCTCTTCA |
| 1405 | YANG-2222 | VH domain | QIQLVQSGAEVKKPGASVKFSCKTSGYTFTNYYMVWVRQAPGQG |
| | | amino | LEWMGIIKPSGGNTIYAQNFQGRVTMTRDTSTNTVYMDLRSLRS |
| | | acid | EDTAVYYCARTGDRAFDVWGQGTMVTVSS |
| | | sequence | |
| 1406 | YANG-2222 | HCDR1 | GYTFTNYY |
| 1407 | YANG-2222 | HCDR2 | IKPSGGNT |
| 1408 | YANG-2222 | HCDR3 | ARTGDRAFDV |
| 1409 | YANG-2222 | VL domain | CAGTCTGTGTTGACGCAGCCGCCCTCAGTATCTGCGACCCCAGG |
| | | nucleic | ACAGAAGGTCACCATCTCCTGCTCTGGAAGCAGCTCCAACATTG |
| | | acid | GGAATAATTTTGTATCCTGGTACCAGCAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGCTGACCGTCCTA |
| 1410 | YANG-2222 | VL domain | QSVLTQPPSVSATPGQKVTISCSGSSSNIGNNFVSWYQQFPGTA |
| | | amino | PKLLIYDNNKRPSGIPDRFSGSKSGTSATLGITGLQTGDEADYY |
| | | acid | CGTWDSSLGAGVFGGGTKLTVL |
| | | sequence | |
| 1411 | YANG-2222 | LCDR1 | SSNIGNNF |
| 1412 | YANG-2222 | LCDR2 | DNN |
| 1413 | YANG-2222 | LCDR3 | GTWDSSLGAGV |
| 1414 | YANG-2223 | VH domain | CAGATGCAGCTGGTACAGTCTGGGGCTGAGGTGAAGAAGCCTGG |
| | | nucleic | GGCCTCAGTGAAGATTTCCTGCAAGACATCTGGATACACCTTCA |
| | | acid | CCAGCTACTATATGGTCTGGGTGCGACAGGCCCCTGGACAAGGA |
| | | sequence | CTTGAGTGGATGGGCATAATCAAACCTAGTGGTGGTAACACAAT |
| | | | CTATGCACAGAAGTTCCAGGGCAGAGTCACCATGACCAGGGACA |
| | | | CGTCCACGAACACAGTCTACATGGACCTGAGTAGTCTGAGATCT |
| | | | GAGGACACGGCCGTGTATTATTGTGCGAGAACTGGGGATAGGGC |
| | | | TTTTGATGTCTGGGGCCAAGGGACAATGGTCACCGTCTCTTCA |
| 1415 | YANG-2223 | VH domain | QMQLVQSGAEVKKPGASVKISCKTSGYTFTSYYMVWVRQAPGQG |
| | | amino | LEWMGIIKPSGGNTIYAQKFQGRVTMTRDTSTNTVYMDLSSLRS |
| | | acid | EDTAVYYCARTGDRAFDVWGQGTMVTVSS |
| | | sequence | |
| 1416 | YANG-2223 | HCDR1 | GYTFTSYY |
| 1417 | YANG-2223 | HCDR2 | IKPSGGNT |
| 1418 | YANG-2223 | HCDR3 | ARTGDRAFDV |
| 1419 | YANG-2223 | VL domain | CAGTCTGTGTTGACGCAGCCGCCCTCAGTGTCTGCGGCCCCAGG |
| | | nucleic | ACAGAAGGTCACCATCTCCTGCTCTGGAAGTACCTCCAACATTG |
| | | acid | GGAGTAATTATGTCTCCTGGTACCAGCAGCTCCCAGGAACAGCC |
| | | sequence | CCCAAACTCCTCATTTATGACAATAGTAAGCGCCCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAACTGACCGTCCTA |
| 1420 | YANG-2223 | VL domain | QSVLTQPPSVSAAPGQKVTISCSGSTSNIGSNYVSWYQQLPGTA |
| | | amino | PKLLIYDNSKRPSGIPDRFSGSKSGTSATLGITGLQTGDEADYY |
| | | acid | CGTWDSSLGAGVFGGGTKLTVL |
| | | sequence | |
| 1421 | YANG-2223 | LCDR1 | TSNIGSNY |
| 1422 | YANG-2223 | LCDR2 | DNS |
| 1423 | YANG-2223 | LCDR3 | GTWDSSLGAGV |
| 1424 | YANG-2224 | VH domain | CAGATACAGCTGGTGCAGTCTGGGGCTGAGGTGAAGAAGCCTGG |
| | | nucleic | GGCCTCAGTGAAGTTTTCCTGCAAGACATCTGGATACACCTTCA |
| | | acid | CCAACTACTATATGGTCTGGGTGCGACAGGCCCCTGGACAAGGA |
| | | sequence | CTTGAGTGGATGGGCATAATCAAACCTAGTGGTGGTAACACAAT |
| | | | CTACGCACAGAACTTCCAGGGCAGAGTCACCATGACCAGGGACA |
| | | | CGTCCACGAACACAGTCTACATGGACCTGAGGAGTCTGAGATCT |
| | | | GAGGACACGGCCGTGTATTATTGTGCGAGAACTGGTGATAGGGC |
| | | | TTTTGATGTCTGGGGCCAAGGGACAATGGTCACCGTCTCTTCA |
| 1425 | YANG-2224 | VH domain | QIQLVQSGAEVKKPGASVKFSCKTSGYTFTNYYMVWVRQAPGQG |
| | | amino | LEWMGIIKPSGGNTIYAQNFQGRVTMTRDTSINTVYMDLRSLRS |
| | | acid | EDTAVYYCARTGDRAFDVWGQGTMVTVSS |
| | | sequence | |
| 1426 | YANG-2224 | HCDR1 | GYTFTNYY |
| 1427 | YANG-2224 | HCDR2 | IKPSGGNT |
| 1428 | YANG-2224 | HCDR3 | ARTGDRAFDV |
| 1429 | YANG-2224 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTTTGTATCCTGGTACCAGCAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGTTTGGGTGCTGGGGTGTTCGGCGG |
| | | | AGGGACCAAACTGACCGTCCTA |
| 1430 | YANG-2224 | VL domain | QSVLTQPPSVSAAPGQKVTISCSGSSSNIGNNFVSWYQQFPGTA |
| | | amino | PKLLIYDNNKRPSGIPDRFSGSKSGTSATLGITGLQTGDEADYY |
| | | acid | CGTWDSSLGAGVFGGGTKLTVL |
| | | sequence | |
| 1431 | YANG-2224 | LCDR1 | SSNIGNNF |
| 1432 | YANG-2224 | LCDR2 | DNN |
| 1433 | YANG-2224 | LCDR3 | GTWDSSLGAGV |
| 1434 | YANG-2225 | VH domain | CAGATTCAGTTAGTGCAGTCTGGGGCTGAGGTGAAGAAGCCTGG |
| | | nucleic | GGCCTCAGTGAAGCTTTCCTGCAAGACATCTGGATACACCTTCA |
| | | acid | CCAGCTACTATATGGTCTGGGTGCGACAGGCCCCTGGACAAGGA |
| | | sequence | CTTGAGTGGATGGGCATAATCAAACCTAGTGGTGGTAACACAAT |
| | | | CTACGCACAGAAGTTCCAGGGCAGAGTCACCATGACCAGGGACA |
| | | | CGTCCACGAACACAGTCTACATGGACCTGAACAGTCTGAGATCT |
| | | | GAGGACACGGCCGTCTATTATTGTGCGAGAACTGGAGATAGGGC |
| | | | TTTTGATGTCTGGGGCCACGGGACAATGGTCACCGTGTCTTCA |
| 1435 | YANG-2225 | VH domain | QIQLVQSGAEVKKPGASVKLSCKTSGYTFTSYYMVWVRQAPGQG |
| | | amino | LEWMGIIKPSGGNTIYAQKFQGRVTMTRDTSTNTVYMDLNSLRS |
| | | acid | EDTAVYYCARTGDRAFDVWGHGTMVTVSS |
| | | sequence | |
| 1436 | YANG-2225 | HCDR1 | GYTFTSYY |
| 1437 | YANG-2225 | HCDR2 | IKPSGGNT |
| 1438 | YANG-2225 | HCDR3 | ARTGDRAFDV |
| 1439 | YANG-2225 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAATATTG |
| | | acid | GGAATAATTATGTATCCTGGTACCAGCAGTTCCCAGGAACAGCC |
| | | sequence | CCCAAACTCTTCATTTATGACAATAATAAGCGACCCTCAGGGAT |
| | | | TTCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGCTGACCGTCCTA |
| 1440 | YANG-2225 | VL domain | QSVLTQPPSVSAAPGQKVTISCSGSSSNIGNNYVSWYQQFPGTA |
| | | amino | PKLFIYDNNKRPSGISDRFSGSKSGTSATLGITGLQTGDEADYY |
| | | acid | CGTWDSSLGAGVFGGGTKLTVL |
| | | sequence | |
| 1441 | YANG-2225 | LCDR1 | SSNIGNNY |
| 1442 | YANG-2225 | LCDR2 | DNN |
| 1443 | YANG-2225 | LCDR3 | GTWDSSLGAGV |
| 1444 | YANG-2226 | VH domain | CAGATACAGCTGGTACAGTCTGGGGCTGAGGTGAAGAAGCCTGG |
| | | nucleic | GGCCTCAGTGAAGTTTTCCTGCAAGACATCTGGATACACCTTCA |
| | | acid | CCACCAACTATATGGTCTGGGTGCGACAGGCCCCTGGACAAGGA |
| | | sequence | CTTGAGTGGATGGGCATAATCAAACCTAGTGGTGGTGACACAAT |
| | | | CTACGCACAGCAATTCCAGGGCAGAGTCACCATGACCAGGGACA |
| | | | CGTCCACGAACACAGTCTACATGGACCTGACCAGTCTGACATCT |
| | | | GAGGACACGGCCGTCTATTATTGTGCGAGAACTGGGGATAGGGC |
| | | | TTTTGATGTCTGGGGCCAAGGGACAATGGTCACCGTCTCGTCA |
| 1445 | YANG-2226 | VH domain | QIQLVQSGAEVKKPGASVKFSCKTSGYTFTTNYMVWVRQAPGQG |
| | | amino | LEWMGIIKPSGGDTIYAQQFQGRVTMTRDTSTNTVYMDLTSLTS |
| | | acid | EDTAVYYCARTGDRAFDVWGQGTMVTVSS |
| | | sequence | |
| 1446 | YANG-2226 | HCDR1 | GYTFTTNY |
| 1447 | YANG-2226 | HCDR2 | IKPSGGDT |
| 1448 | YANG-2226 | HCDR3 | ARTGDRAFDV |
| 1449 | YANG-2226 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTATGTATCCTGGTACCAGCAGCTCCCAGGAACAGCC |
| | | sequence | CCCAAACTCCTCATTTATGACGATGGAAAGCGACCCTCAGGGAT |
| | | | TCCTGATCGATTCTCTGGCTCCAAGTCTGGCACGTCAGCCTCCC |
| | | | TGGGCATCACCGGACTCCAGACTGGGGACGAGGCCGATTATTAC |
| | | | TGCGGAACATGGGATACCAGCCTGGGTTCTGGGGTGTTCGGCGG |
| | | | CGGGACCAAGTTGACCGTCCTA |
| 1450 | YANG-2226 | VL domain | QSVLTQPPSVSAAPGQKVTISCSGSSSNIGNNYVSWYQQLPGTA |
| | | amino | PKLLIYDDGKRPSGIPDRFSGSKSGTSASLGITGLQTGDEADYY |
| | | acid | CGTWDTSLGSGVFGGGTKLTVL |
| | | sequence | |
| 1451 | YANG-2226 | LCDR1 | SSNIGNNY |
| 1452 | YANG-2226 | LCDR2 | DDG |
| 1453 | YANG-2226 | LCDR3 | GTWDTSLGSGV |
| 1454 | YANG-2227 | VH domain | CAGATACAACTGGTGCAGTCTGGGGCTGAGGTGAAGAAGCCTGG |
| | | nucleic | GGCCTCAGTGAAGTTTTCCTGCAAGACATCTGGATACACCTTCA |
| | | acid | CCAACTACTATATGGTCTGGGTGCGACAGGCCCCTGGACAAGGA |
| | | sequence | CTTGAGTGGATGGGCATAATCAAACCTAGTGGTGGTGACACAAT |
| | | | CTACGCACAGAACTTCCAGGGCAGAGTCACCATGACCAGGGACA |
| | | | CGTCCACGAACACAGTCTATATGGACCTGAGGAGTCTGAGATCT |
| | | | GAAGACACGGCCGTGTATTATTGTGCGAGAACTGGTGATAGGGC |
| | | | TTTTGATGTCTGGGGCCAAGGGACAATGGTCACCGTCTCTTCA |
| 1455 | YANG-2227 | VH domain | QIQLVQSGAEVKKPGASVKFSCKTSGYTFTNYYMVWVRQAPGQG |
| | | amino | LEWMGIIKPSGGDTIYAQNFQGRVTMTRDTSTNTVYMDLRSLRS |
| | | acid | EDTAVYYCARTGDRAFDVWGQGTMVTVSS |
| | | sequence | |
| 1456 | YANG-2227 | HCDR1 | GYTFTNYY |
| 1457 | YANG-2227 | HCDR2 | IKPSGGDT |
| 1458 | YANG-2227 | HCDR3 | ARTGDRAFDV |
| 1459 | YANG-2227 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTTTGTATCCTGGTACCAGCAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGCTGACCGTCCTA |
| 1460 | YANG-2227 | VL domain | QSVLTQPPSVSAAPGQKVTISCSGSSSNIGNNFVSWYQQFPGTA |
| | | amino | PKLLIYDNNKRPSGIPDRFSGSKSGTSATLGITGLQTGDEADYY |
| | | acid | CGTWDSSLGAGVFGGGTKLTVL |
| | | sequence | |
| 1461 | YANG-2227 | LCDR1 | SSNIGNNF |
| 1462 | YANG-2227 | LCDR2 | DNN |
| 1463 | YANG-2227 | LCDR3 | GTWDSSLGAGV |
| 1464 | YANG-2228 | VH domain | CAGATGCAATTGGTGCAATCTGGGGCTGAGGTGAAGAAGCCTGG |
| | | nucleic | GGCCTCAGTGAAGATTTCCTGCAAGACATCTGGATACACCTTCA |
| | | acid | CCAACTACTATATGGTCTGGGTGCGACAGGCCCCTGGACAAGGA |
| | | sequence | CTTGAGTGGATGGGCATAATCAAACCTAGTGGTGGTAACACGAT |
| | | | CTACGCACAGAAGTTCCAGGACAGAGTCACCATGACCAGGGACA |
| | | | CGTCCACGAACACAGTCTATTTGGACCTGAGCAGTCTGAGATCT |
| | | | GAGGACACGGCCGTCTATTATTGTGCGAGAACTGGGGATAGGGC |
| | | | TTTTGATGTCTGGGGCCACGGGACAATGGTCACCGTCTCTTCA |
| 1465 | YANG-2228 | VH domain | QMQLVQSGAEVKKPGASVKISCKTSGYTFTNYYMVWVRQAPGQG |
| | | amino | LEWMGIIKPSGGNTIYAQKFQDRVTMTRDTSTNTVYLDLSSLRS |
| | | acid | EDTAVYYCARTGDRAFDVWGHGTMVTVSS |
| | | sequence | |
| 1466 | YANG-2228 | HCDR1 | GYTFTNYY |
| 1467 | YANG-2228 | HCDR2 | IKPSGGNT |
| 1468 | YANG-2228 | HCDR3 | ARTGDRAFDV |
| 1469 | YANG-2228 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTTTGTTTCCTGGTACCAGCAGTTCCCAGGAACAGCC |
| | | sequence | CCCAAACTCCTCATTTATGTCAATAATAAGCGACCCTCAGGGAT |
| | | | TCCTGACCGATTCTCTGGCTCCAGGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAACTGACCGTCCTA |
| 1470 | YANG-2228 | VL domain | QSVLTQPPSVSAAPGQKVTISCSGSSSNIGNNFVSWYQQFPGTA |
| | | amino | PKLLIYVNNKRPSGIPDRESGSRSGTSATLGITGLQTGDEADYY |
| | | acid | CGTWDSSLGAGVFGGGTKLTVL |
| | | sequence | |
| 1471 | YANG-2228 | LCDR1 | SSNIGNNF |
| 1472 | YANG-2228 | LCDR2 | VNN |
| 1473 | YANG-2228 | LCDR3 | GTWDSSLGAGV |
| 1474 | YANG-2229 | VH domain | CAGATGCAGTTGGTGCAGTCTGGGGCTGAGGTGAGGAAGCCTGG |
| | | nucleic | GGCCTCAGTGAAGATTTCCTGCAAGACATCTGGATACACCTTCA |
| | | acid | TCAACTACTATATGGTCTGGGTGCGACAGGCCCCTGGACAAGGA |
| | | sequence | CTTGAGTGGATGGGCATAATCAAACCTAGTGGTGGTTATACGAT |
| | | | CTACGCACAGAAGTTCCAGGGCAGAGTCACCATGACCAGGGACA |
| | | | CGTCCACGTACACAGTCTATATGGCCCTGAGCGGTCTGAGATCT |
| | | | GAGGACACGGCCGTGTATTATTGTGCGAGAACTGGGGATAGGGC |
| | | | TTTTGATGTCTGGGGCCACGGGACAATGGTCACCGTCTCTTCA |
| 1475 | YANG-2229 | VH domain | QMQLVQSGAEVRKPGASVKISCKTSGYTFINYYMVWVRQAPGQG |
| | | amino | LEWMGIIKPSGGYTIYAQKFQGRVTMTRDTSTYTVYMALSGLRS |
| | | acid | EDTAVYYCARTGDRAFDVWGHGTMVTVSS |
| | | sequence | |
| 1476 | YANG-2229 | HCDR1 | GYTFINYY |
| 1477 | YANG-2229 | HCDR2 | IKPSGGYT |
| 1478 | YANG-2229 | HCDR3 | ARTGDRAFDV |
| 1479 | YANG-2229 | VL domain | CAGTCTGTATTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTATGTTTCCTGGTACCAGCAGTTCCCAGGAACAGCC |
| | | sequence | CCCAAACTCCTCATTTATGACAATCATAAGCGACCCTCAGGTAT |
| | | | TCCTGACCGATTCTCTGCCTCCAAGTCTGGCACGTCAGCCACCC |
| | | | TGGGCATCACCGGACTCCTGACTGGGGACGAGGCCGATTATTAC |
| | | | TGCGGAACATGGGATAGCGGCCTGGGTGCTGGGGTGTTCGGCGG |
| | | | AGGGACCAAGCTGACCGTCCTA |
| 1480 | YANG-2229 | VL domain | QSVLTQPPSVSAAPGQKVTISCSGSSSNIGNNYVSWYQQFPGTA |
| | | amino | PKLLIYDNHKRPSGIPDRESASKSGTSATLGITGLLTGDEADYY |
| | | acid | CGTWDSGLGAGVFGGGTKLTVL |
| | | sequence | |
| 1481 | YANG-2229 | LCDR1 | SSNIGNNY |
| 1482 | YANG-2229 | LCDR2 | DNH |
| 1483 | YANG-2229 | LCDR3 | GTWDSGLGAGV |
| 1484 | YANG-2230 | VH domain | CAGATTCAATTGGTGCAGTCTGGGGCTGAGGTGAAGAAGCCTGG |
| | | nucleic | GGCCTCCGTGAAGATTTCGTGCAAGCCATCTGGATACACCTTCA |
| | | acid | CCAACTACTATATGGTCTGGGTGCGACAGGCCCCTGGACAAGGA |
| | | sequence | CTTGAGTGGATGGGCATAATCAAACCTAGTGGTGGTAACACGAT |
| | | | CTACGCACAGAAGTTCCAGGGCAGAGTCACCATGACCAGGGACA |
| | | | CGTCCACGAACACAGTCTATTTGGACCTGAGCAGTCTGAGATCT |
| | | | GAGGACACGGCCGTATATTATTGTGCGAGAACTGGGGATAGGGC |
| | | | TTTTGATGTCTGGGGCCACGGGACAATGGTCACCGTCTCTTCA |
| 1485 | YANG-2230 | VH domain | QIQLVQSGAEVKKPGASVKISCKPSGYTFTNYYMVWVRQAPGQG |
| | | amino | LEWMGIIKPSGGNTIYAQKFQGRVTMTRDTSTNTVYLDLSSLRS |
| | | acid | EDTAVYYCARTGDRAFDVWGHGTMVTVSS |
| | | sequence | |
| 1486 | YANG-2230 | HCDR1 | GYTFTNYY |
| 1487 | YANG-2230 | HCDR2 | IKPSGGNT |
| 1488 | YANG-2230 | HCDR3 | ARTGDRAFDV |
| 1489 | YANG-2230 | VL domain | CAGTCTGTGTTGACGCAGACGCCCTCAGTGTCTGCGGCCCCAGG |
| | | nucleic | ACAGAAGGTCACCATCTCCTGCTCTGGAAGCAGCTCCAATATTG |
| | | acid | GGAATAATTATGTTTCCTGGTACCAGCAGTTCCCAGGAACAGTC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGAATATTAC |
| | | | TGCGGAACATGGGATAGCAGCCTGGGTGCTGGGGTGTTCGGCGG |
| | | | AGGGACCAAGCTGACCGTCCTA |
| 1490 | YANG-2230 | VL domain | QSVLTQTPSVSAAPGQKVTISCSGSSSNIGNNYVSWYQQFPGTV |
| | | amino | PKLLIYDNNKRPSGIPDRFSGSKSGTSATLGITGLQTGDEAEYY |
| | | acid | CGTWDSSLGAGVFGGGTKLTVL |
| | | sequence | |
| 1491 | YANG-2230 | LCDR1 | SSNIGNNY |
| 1492 | YANG-2230 | LCDR2 | DNN |
| 1493 | YANG-2230 | LCDR3 | GTWDSSLGAGV |
| 1494 | YANG-2231 | VH domain | CAGATTCAGTTGGTGCAGTCTGGGGCTGAGGTGAAGAAGCCTGG |
| | | nucleic | GGCCTCAGTGAAGTTTTCCTGCAAGACATCTGGATACACCTTCA |
| | | acid | CCAGCTACTATATGGTCTGGGTGCGACAGGCCCCTGGACAAGGA |
| | | sequence | CTTGAGTGGATGGGCATTATCAAACCTAGTGGTGGTAACACAAT |
| | | | CTACGCACAGAAGTTCCAGGGCAGAGTCACCATGACCAGGGACA |
| | | | CGTCCACGAACACAGTCTACATGGACCTGAGCAGTCTGAGATCT |
| | | | GAGGACACGGCCGTTTATTATTGTGCGAGAACTGGGGATAGGGC |
| | | | TTTTGATGTCTGGGGCCACGGGACAATGATCACCGTCTCTTCA |
| 1495 | YANG-2231 | VH domain | QIQLVQSGAEVKKPGASVKFSCKTSGYTFTSYYMVWVRQAPGQG |
| | | amino | LEWMGIIKPSGGNTIYAQKFQGRVTMTRDTSTNTVYMDLSSLRS |
| | | acid | EDTAVYYCARTGDRAFDVWGHGTMITVSS |
| | | sequence | |
| 1496 | YANG-2231 | HCDR1 | GYTFTSYY |
| 1497 | YANG-2231 | HCDR2 | IKPSGGNT |
| 1498 | YANG-2231 | HCDR3 | ARTGDRAFDV |
| 1499 | YANG-2231 | VL domain | CAGTCTGTATTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTTTGTATCCTGGTACCAACAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGCTGACCGTCCTA |
| 1500 | YANG-2231 | VL domain | QSVLTQPPSVSAAPGQKVTISCSGSSSNIGNNFVSWYQQFPGTA |
| | | amino | PKLLIYDNNKRPSGIPDRFSGSKSGTSATLGITGLQTGDEADYY |
| | | acid | CGTWDSSLGAGVFGGGTKLTVL |
| | | sequence | |
| 1501 | YANG-2231 | LCDR1 | SSNIGNNE |
| 1502 | YANG-2231 | LCDR2 | DNN |
| 1503 | YANG-2231 | LCDR3 | GTWDSSLGAGV |
| 1504 | YANG-2232 | VH domain | CAGATACAGCTGGTGCAGTCTGGGGCTGAGGTGAAGAAGCCTGG |
| | | nucleic | GGCCTCAGTGAAGTTTTCCTGCAAGACATCTGGATACACCTTCA |
| | | acid | CCAACTACTATATGGTCTGGGTGCGACAGGCCCCTGGACAAGGA |
| | | sequence | CTTGAGTGGATGGGCATAATCAAACCTAGTGGTGGTAACACAAT |
| | | | CTACGCACAGAATTTCCAGGGCAGAGTCACCATGACCAGGGACA |
| | | | CGTCCACGAACACAGTCTACATGGACCTGAGGAGTCTGAGATCT |
| | | | GAGGACACGGCCGTGTATTATTGTGCGAGAACTGGTGATAGGGC |
| | | | TTTTGATGTCTGGGGCCAAGGGACAATGGTCACCGTCTCTGCA |
| 1505 | YANG-2232 | VH domain | QIQLVQSGAEVKKPGASVKFSCKTSGYTFTNYYMVWVRQAPGQG |
| | | amino | LEWMGIIKPSGGNTIYAQNFQGRVTMTRDTSTNTVYMDLRSLRS |
| | | acid | EDTAVYYCARTGDRAFDVWGQGTMVTVSA |
| | | sequence | |
| 1506 | YANG-2232 | HCDR1 | GYTFTNYY |
| 1507 | YANG-2232 | HCDR2 | IKPSGGNT |
| 1508 | YANG-2232 | HCDR3 | ARTGDRAFDV |
| 1509 | YANG-2232 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTTTGTATCCTGGTACCAGCAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGCTGACCGTCCTA |
| 1510 | YANG-2232 | VL domain | QSVLTQPPSVSAAPGQKVTISCSGSSSNIGNNFVSWYQQFPGTA |
| | | amino | PKLLIYDNNKRPSGIPDRFSGSKSGTSATLGITGLQTGDEADYY |
| | | acid | CGTWDSSLGAGVFGGGTKLTVL |
| | | sequence | |
| 1511 | YANG-2232 | LCDR1 | SSNIGNNF |
| 1512 | YANG-2232 | LCDR2 | DNN |
| 1513 | YANG-2232 | LCDR3 | GTWDSSLGAGV |
| 1514 | YANG-2233 | VH domain | CAGATGCAACTGGTGCAGTCTGGGGCTGAGGTGAAGAAGCCTGG |
| | | nucleic | GGCCTCAGTGAAGATTTCCTGCAAGACATCTGGATACCCCTTCA |
| | | acid | CCAACTACTATATGGTCTGGGTGCGACAGGCCCCTGGACAAGGA |
| | | sequence | CTTGAGTGGATGGGCATAATCAAACCTAGTGGTGGTAACACGAT |
| | | | CTATGCACAGAAGTTCCAGGGCAGAGTCACCATGACCAGGGACA |
| | | | CGTCCACCAACACAGTCTATTTGGACCTGAGCAGTCTGAGATCT |
| | | | GAGGACACGGCCGTGTATTTTTGTGCGAGAACTGGGGATAGGGC |
| | | | TTTTGATGTCTGGGGCCACGGGACAATAGTCACCGTCTCTTCA |
| 1515 | YANG-2233 | VH domain | QMQLVQSGAEVKKPGASVKISCKTSGYPFTNYYMVWVRQAPGQG |
| | | amino | LEWMGIIKPSGGNTIYAQKFQGRVTMTRDTSTNTVYLDLSSLRS |
| | | acid | EDTAVYFCARTGDRAFDVWGHGTIVTVSS |
| | | sequence | |
| 1516 | YANG-2233 | HCDR1 | GYPFTNYY |
| 1517 | YANG-2233 | HCDR2 | IKPSGGNT |
| 1518 | YANG-2233 | HCDR3 | ARTGDRAFDV |
| 1519 | YANG-2233 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTATGTTTCCTGGTACCAGCAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGCTGACCGTCCTA |
| 1520 | YANG-2233 | VL domain | QSVLTQPPSVSAAPGQKVTISCSGSSSNIGNNYVSWYQQFPGTA |
| | | amino | PKLLIYDNNKRPSGIPDRESGSKSGTSATLGITGLQTGDEADYY |
| | | acid | CGTWDSSLGAGVFGGGTKLTVL |
| | | sequence | |
| 1521 | YANG-2233 | LCDR1 | SSNIGNNY |
| 1522 | YANG-2233 | LCDR2 | DNN |
| 1523 | YANG-2233 | LCDR3 | GTWDSSLGAGV |
| 1524 | YANG-2234 | VH domain | CAGATGCAATTGGTGCAGTCTGGGACTGAGGTGAAGAAGCCTGG |
| | | nucleic | GGCCTCAGTGAAGATTTCCTGCAAGACATCTGGATACACCTTCA |
| | | acid | CCAACTACTATATGGTCTGGGTGCGACAGGCCCCTGGACAAGGA |
| | | sequence | CTTGAGTGGATGGGCATAATCAAACCTAGTGGTGGTAACACGAT |
| | | | CTACGCACAGAAGTTCCAGGGCAGAGTCACCATGACCAGGGACA |
| | | | CGTCCACGAACACAGTCTATTTGGACCTGAGCAGTCTGAGATCT |
| | | | GAGGACACGGCCATGTATTATTGTGCGAGAACTGGTGATAGGGC |
| | | | TTTTGATGTCTGGGGCCACGGGACAGTGGTCACCGTCTCTTCA |
| 1525 | YANG-2234 | VH domain | QMQLVQSGTEVKKPGASVKISCKTSGYTFTNYYMVWVRQAPGQG |
| | | amino | LEWMGIIKPSGGNTIYAQKFQGRVTMTRDTSTNTVYLDLSSLRS |
| | | acid | EDTAMYYCARTGDRAFDVWGHGTVVTVSS |
| | | sequence | |
| 1526 | YANG-2234 | HCDR1 | GYTFTNYY |
| 1527 | YANG-2234 | HCDR2 | IKPSGGNT |
| 1528 | YANG-2234 | HCDR3 | ARTGDRAFDV |
| 1529 | YANG-2234 | VL domain | CAGTCTGTGCTGACGCAGCCGCCCTCAGTGTTTGCGGCCCCAGG |
| | | nucleic | ACAGAAGGTCACCATCTCCTGCTCTGGAAGCAGCTCCAACATTG |
| | | acid | GGAATAATTATGTTTCCTGGTACCAACAGTTCCCAGGAACAGCC |
| | | sequence | CCCAAACTCCTCATCTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGCTGACCGTCCTA |
| 1530 | YANG-2234 | VL domain | QSVLTQPPSVFAAPGQKVTISCSGSSSNIGNNYVSWYQQFPGTA |
| | | amino | PKLLIYDNNKRPSGIPDRFSGSKSGTSATLGITGLQTGDEADYY |
| | | acid | CGTWDSSLGAGVFGGGTKLTVL |
| | | sequence | |
| 1531 | YANG-2234 | LCDR1 | SSNIGNNY |
| 1532 | YANG-2234 | LCDR2 | DNN |
| 1533 | YANG-2234 | LCDR3 | GTWDSSLGAGV |
| 1534 | YANG-2235 | VH domain | CAGATACAGCTGGTGCAGTCTGGGGCTGAGGTGAAGAAGCCTGG |
| | | nucleic | GGCCTCAGTGAAGCTTTCCTGCAAGACATCTGGATACACCTTCA |
| | | acid | CCAACTACTATATGGTCTGGGTGCGACAGGCCCCTGGACAAGGA |
| | | sequence | CTTGAGTGGATGGGCATAATCAAACCTACTGGTGGTAACACAAT |
| | | | CTACGCACAGAATTTCCAGGGCAGAGTCACCATGTCCAGGGACA |
| | | | CGTCCACGAACACAGTCTACATGGACCTGAGGAGTCTGAGATCT |
| | | | GAGGACGCGGCCGTGTATTATTGTGCGAGAACTGGTGATAGGGC |
| | | | TTTTGATGTCTGGGGCCAAGGGACAATGGTCACCGTCTCTTCA |
| 1535 | YANG-2235 | VH domain | QIQLVQSGAEVKKPGASVKLSCKTSGYTFTNYYMVWVRQAPGQG |
| | | amino | LEWMGIIKPTGGNTIYAQNFQGRVTMSRDTSTNTVYMDLRSLRS |
| | | acid | EDAAVYYCARTGDRAFDVWGQGTMVTVSS |
| | | sequence | |
| 1536 | YANG-2235 | HCDR1 | GYTFTNYY |
| 1537 | YANG-2235 | HCDR2 | IKPTGGNT |
| 1538 | YANG-2235 | HCDR3 | ARTGDRAFDV |
| 1539 | YANG-2235 | VL domain | CAGTCTGTGTTGACGCAGCCGCCCTCAGTGTCTGCGACCCCAGG |
| | | nucleic | ACAGAAGGTCACCATCTCCTGCTCTGGAAGCAGCTCCAACATTG |
| | | acid | GGAATAATTTTGTATCCTGGTACCAGCAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCGGCCTGGGTGCTGGGGTGTTCGGCGG |
| | | | AGGGACCAAGCTGACCGTCCTA |
| 1540 | YANG-2235 | VL domain | QSVLTQPPSVSATPGQKVTISCSGSSSNIGNNFVSWYQQFPGTA |
| | | amino | PKLLIYDNNKRPSGIPDRFSGSKSGTSATLGITGLQTGDEADYY |
| | | acid | CGTWDSGLGAGVFGGGTKLTVL |
| | | sequence | |
| 1541 | YANG-2235 | LCDR1 | SSNIGNNF |
| 1542 | YANG-2235 | LCDR2 | DNN |
| 1543 | YANG-2235 | LCDR3 | GTWDSGLGAGV |
| 1544 | YANG-2236 | VH domain | CAGATACAACTGGTGCAGTCTGGGGCTGAGGTGAAGAAGCCTGG |
| | | nucleic | GGCCTCAGTGAAGTTTTCCTGCAAGACATCTGGATACACCTTCA |
| | | acid | CCAACTACTATTTGGTCTGGGTGCGACAGGCCCCTGGACAAGGA |
| | | sequence | CTTGAGTGGATGGGCATAATCAAACCTACTGGTGGTAACACAAT |
| | | | CTACGCACAGAACTTCCAGGGCAGAGTCACCATGACCAGGGACA |
| | | | CGTCCACGAACACAGTCTACATGGACCTGAGGAGTCTGAGATCT |
| | | | GAGGACACGGCCGTGTATTATTGTGCGAGAACTGGTGGTAGGGC |
| | | | TTTTGATGTCTGGGGCCAAGGGACAATGGTCACCGTCTCTTCA |
| 1545 | YANG-2236 | VH domain | QIQLVQSGAEVKKPGASVKFSCKTSGYTFTNYYLVWVRQAPGQG |
| | | amino | LEWMGIIKPTGGNTIYAQNFQGRVTMTRDTSTNTVYMDLRSLRS |
| | | acid | EDTAVYYCARTGGRAFDVWGQGTMVTVSS |
| | | sequence | |
| 1546 | YANG-2236 | HCDR1 | GYTFTNYY |
| 1547 | YANG-2236 | HCDR2 | IKPTGGNT |
| 1548 | YANG-2236 | HCDR3 | ARTGGRAFDV |
| 1549 | YANG-2236 | VL domain | CAGTCTGTGTTGACGCAGCCGCCCTCAGTGTCTGCGGCCCCAGG |
| | | nucleic | ACAGAAGGTCACCATCTCCTGCTCTGGAAACAGCTCCAATATTG |
| | | acid | GGGATAATTTTGTGTCCTGGTACCAACAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAGGCTGACCGTCCTA |
| 1550 | YANG-2236 | VL domain | QSVLTQPPSVSAAPGQKVTISCSGNSSNIGDNFVSWYQQFPGTA |
| | | amino | PKLLIYDNNKRPSGIPDRFSGSKSGTSATLGITGLQTGDEADYY |
| | | acid | CGTWDSSLGAGVFGGGTRLTVL |
| | | sequence | |
| 1551 | YANG-2236 | LCDR1 | SSNIGDNE |
| 1552 | YANG-2236 | LCDR2 | DNN |
| 1553 | YANG-2236 | LCDR3 | GTWDSSLGAGV |
| 1554 | YANG-2237 | VH domain | CAGATTCAGTTGGTGCAGTCTGGGGCTGAGGTGAAGAAGCCTGG |
| | | nucleic | GGCCTCAGTGAAGTTTTCTTGCAAGACATCTGGATACACCTTCA |
| | | acid | CCAACTACTATATGGTCTGGGTGCGACAGGCCCCTGGACAAGGA |
| | | sequence | CTTGAGTGGATGGGCATAATCAAACCTAGTGGTGGTAACACAAT |
| | | | CTACGCACAGAAGTTCCAGGGCAGAGTCACCATGACCAGGGACA |
| | | | CGTCCACGGACACAGTCTACATGGACCTGAACAGTCTGAGATCT |
| | | | GAGGACACGGCCGTCTATTATTGTGCGAGAACTGGAGATAGGGC |
| | | | TTTTGATGTCTGGGGCCACGGGACAATGGTCACCGTGTCTTCA |
| 1555 | YANG-2237 | VH domain | QIQLVQSGAEVKKPGASVKFSCKTSGYTFTNYYMVWVRQAPGQG |
| | | amino | LEWMGIIKPSGGNTIYAQKFQGRVTMTRDTSTDTVYMDLNSLRS |
| | | acid | EDTAVYYCARTGDRAFDVWGHGTMVTVSS |
| | | sequence | |
| 1556 | YANG-2237 | HCDR1 | GYTFTNYY |
| 1557 | YANG-2237 | HCDR2 | IKPSGGNT |
| 1558 | YANG-2237 | HCDR3 | ARTGDRAFDV |
| 1559 | YANG-2237 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTATGTATCCTGGTACCAGCAGTTCCCAGGAACAGCC |
| | | sequence | CCCAAACTCCTCATTTATGACAATAATAAGCGACCCTCAGGGAT |
| | | | TCTTGACCGATTCTCTGGCTCCAAGTCTGGCACGTCAGCCACCC |
| | | | TGGGCATCACCGGACTCCAGACTGGGGACGAGGCCGATTATTAT |
| | | | TGCGGAACATGGGATAGCAGCCTGGGTGCTGGGGTGTTCGGCGG |
| | | | AGGGACCAAACTGACCGTCCTA |
| 1560 | YANG-2237 | VL domain | QSVLTQPPSVSAAPGQKVTISCSGSSSNIGNNYVSWYQQFPGTA |
| | | amino | PKLLIYDNNKRPSGILDRFSGSKSGTSATLGITGLQTGDEADYY |
| | | acid | CGTWDSSLGAGVFGGGTKLTVL |
| | | sequence | |
| 1561 | YANG-2237 | LCDR1 | SSNIGNNY |
| 1562 | YANG-2237 | LCDR2 | DNN |
| 1563 | YANG-2237 | LCDR3 | GTWDSSLGAGV |
| 1564 | YANG-2238 | VH domain | CAGCTGCAATTGGTGCAGTCTGGGGCTGAGGTGAAGAAGCCTGG |
| | | nucleic | GGCCTCAGTGAAGATTTCCTGCAAGACATCTGGATACACCTTCA |
| | | acid | CCAACTACTATATGGTCTGGGTGCGACAGGCCCTTGGACAAGGA |
| | | sequence | CTTGAGTGGATGGGCATAATCAAACCTAGTGGTGGTTACACGAG |
| | | | CTACGCACAGAAGTTCCAGGGCAGAGTCACCATGACCAGGGACA |
| | | | CGTCCACGAACACAGTCTATTTGGACCTGAGTAGTCTGAGATCT |
| | | | GAGGACACGGCCGTGTATTATTGTGCGAGAACTGGTGATAGGGC |
| | | | ATTTGATGTCTGGGGCCACGGGACAGTGGTCACCGTCTCTTCA |
| 1565 | YANG-2238 | VH domain | QLQLVQSGAEVKKPGASVKISCKTSGYTFTNYYMVWVRQALGQG |
| | | amino | LEWMGIIKPSGGYTSYAQKFQGRVTMTRDTSTNTVYLDLSSLRS |
| | | acid | EDTAVYYCARTGDRAFDVWGHGTVVTVSS |
| | | sequence | |
| 1566 | YANG-2238 | HCDR1 | GYTFTNYY |
| 1567 | YANG-2238 | HCDR2 | IKPSGGYT |
| 1568 | YANG-2238 | HCDR3 | ARTGDRAFDV |
| 1569 | YANG-2238 | VL domain | CAGTCTGTGTTGACGCAGCCGCCCTCAGTGTCTGCGGCCCCAGG |
| | | nucleic | ACAGAAGGTCACCCTCTCCTGCTCTGGAAGCAGCTCCAACATTG |
| | | acid | GGAATAATTATGTTTCCTGGTACCAGCAGTTCCCAGGAACAGCC |
| | | sequence | CCCAAACTCCTCATCTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGCTGACCGTCCTA |
| 1570 | YANG-2238 | VL domain | QSVLTQPPSVSAAPGQKVTLSCSGSSSNIGNNYVSWYQQFPGTA |
| | | amino | PKLLIYDNNKRPSGIPDRFSGSKSGTSATLGITGLQTGDEADYY |
| | | acid | CGTWDSSLGAGVFGGGTKLTVL |
| | | sequence | |
| 1571 | YANG-2238 | LCDR1 | SSNIGNNY |
| 1572 | YANG-2238 | LCDR2 | DNN |
| 1573 | YANG-2238 | LCDR3 | GTWDSSLGAGV |
| 1574 | YANG-2239 | VH domain | CAGATGCAATTGGTGCAGTCTGGGGCTGAGGTGAAGAAGCCTGG |
| | | nucleic | GGCCTCAGTGAAGATTTCCTGCAAGACATCTGGATACATATTCA |
| | | acid | CCAACTATTATATGGTCTGGGTGCGACAGGCCCCTGGACAAGGA |
| | | sequence | CTTGAGTGGATGGGCATAATCAAACCTAGCGGTGGTAACACGAT |
| | | | CTACGCACAGAAGTTCCAGGGCAGAGTCACCATGACCAGGGACA |
| | | | CGTCCACGAACACAGTCTATTTGGACCTGAGCAGTCTGAGATAT |
| | | | GAGGACACGGCCGTCTATTATTGTGCGAGAACTGGTGATAGGGC |
| | | | TTTTGATGTCTGGGGCCACGGGACAGTGGTCACCGTCTCTTCA |
| 1575 | YANG-2239 | VH domain | QMQLVQSGAEVKKPGASVKISCKTSGYIFTNYYMVWVRQAPGQG |
| | | amino | LEWMGIIKPSGGNTIYAQKFQGRVTMTRDTSTNTVYLDLSSLRY |
| | | acid | EDTAVYYCARTGDRAFDVWGHGTVVTVSS |
| | | sequence | |
| 1576 | YANG-2239 | HCDR1 | GYIFTNYY |
| 1577 | YANG-2239 | HCDR2 | IKPSGGNT |
| 1578 | YANG-2239 | HCDR3 | ARTGDRAFDV |
| 1579 | YANG-2239 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTATGTTTCCTGGTACCAGCAGTTCCCAGGAACAGCC |
| | | sequence | CCCAAACTCCTCATCTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGCTGACCGTCCTA |
| 1580 | YANG-2239 | VL domain | QSVLTQPPSVSAAPGQKVTISCSGSSSNIGNNYVSWYQQFPGTA |
| | | amino | PKLLIYDNNKRPSGIPDRFSGSKSGTSATLGITGLQTGDEADYY |
| | | acid | CGTWDSSLGAGVFGGGTKLTVL |
| | | sequence | |
| 1581 | YANG-2239 | LCDR1 | SSNIGNNY |
| 1582 | YANG-2239 | LCDR2 | DNN |
| 1583 | YANG-2239 | LCDR3 | GTWDSSLGAGV |
| 1584 | YANG-2240 | VH domain | CAGATGCAGTTGGTGCAGTCTGGGGCTGAGGTGAAGAAGCCTGG |
| | | nucleic | GGCCTCAGTGAAGATTTCCTGCAAGACATCTGGATACACCTTCA |
| | | acid | TCAACTACTATATGGTCTGGGTGCGACAGGCCCCTGGACAAGGA |
| | | sequence | CTTGAGTGGATGGGCATAATCAAACCTAGTGGTGGTAACACGAT |
| | | | CTACGCACAGAAGTTCCAGGGCAGAGTCACCATGACCAGGGACA |
| | | | CGTCCACGAACACAGTCTATATGGACCTGAGCAGTCTGAGATCT |
| | | | GAGGACACGGCCGTGTATTATTGTGCGAGAACTGGGGATAGGGC |
| | | | TTTTGATGTCTGGGGCCACGGGACAATGGTCACCGTCTCTTCA |
| 1585 | YANG-2240 | VH domain | QMQLVQSGAEVKKPGASVKISCKTSGYTFINYYMVWVRQAPGQG |
| | | amino | LEWMGIIKPSGGNTIYAQKFQGRVTMTRDTSTNTVYMDLSSLRS |
| | | acid | EDTAVYYCARTGDRAFDVWGHGTMVTVSS |
| | | sequence | |
| 1586 | YANG-2240 | HCDR1 | GYTFINYY |
| 1587 | YANG-2240 | HCDR2 | IKPSGGNT |
| 1588 | YANG-2240 | HCDR3 | ARTGDRAFDV |
| 1589 | YANG-2240 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTTTGTTTCCTGGTACCAGCAGTTCCCAGGAACAGCC |
| | | sequence | CCCAAACTCCTCATTTATGACAATCATAAGCGACCCTCAGGGAT |
| | | | TCCTGACCGATTCTCTGCCTCCAAGTCTGGCACGTCAGCCACCC |
| | | | TGGGCATCACCGGACTCCAGACTGGGGACGAGGCCGATTATTAC |
| | | | TGCGGAACATGGGATAGCGGCCTGGGTGCTGGGGTGTTCGGCGG |
| | | | AGGGACCAAGCTGACCGTCCTA |
| 1590 | YANG-2240 | VL domain | QSVLTQPPSVSAAPGQKVTISCSGSSSNIGNNFVSWYQQFPGTA |
| | | amino | PKLLIYDNHKRPSGIPDRFSASKSGTSATLGITGLQTGDEADYY |
| | | acid | CGTWDSGLGAGVFGGGTKLTVL |
| | | sequence | |
| 1591 | YANG-2240 | LCDR1 | SSNIGNNF |
| 1592 | YANG-2240 | LCDR2 | DNH |
| 1593 | YANG-2240 | LCDR3 | GTWDSGLGAGV |
| 1594 | YANG-2241 | VH domain | CAGATTCAATTGGTGCAGTCTGGGGCTGAGGTGAAGAAGCCTGG |
| | | nucleic | GGCCTCAGTGAAGATTTCCTGCAAGACATCTGGATATATCTTCA |
| | | acid | CCAACTACTATATGGTCTGGGTGCGACAGGCCCCTGGACAAGGA |
| | | sequence | CTTGAGTGGATGGGCATAATCAAACCTAGTGGTGGTAACACGAT |
| | | | CTACGCACAGAAGTTCCAGGGCAGAGTCACCATGACCAGGGACA |
| | | | CGTCCACGACCACAGTCTATTTGGACCTGAACAGTCTGAGATCT |
| | | | GAGGACACGGCCGTGTATTATTGTGCGAGAACTGGTGATAGGGC |
| | | | TTTTGATGTCTGGGGCCACGGGACAGTGGTCACCGTCTCTTCA |
| 1595 | YANG-2241 | VH domain | QIQLVQSGAEVKKPGASVKISCKTSGYIFTNYYMVWVRQAPGQG |
| | | amino | LEWMGIIKPSGGNTIYAQKFQGRVTMTRDTSTTTVYLDLNSLRS |
| | | acid | EDTAVYYCARTGDRAFDVWGHGTVVTVSS |
| | | sequence | |
| 1596 | YANG-2241 | HCDR1 | GYIFTNYY |
| 1597 | YANG-2241 | HCDR2 | IKPSGGNT |
| 1598 | YANG-2241 | HCDR3 | ARTGDRAFDV |
| 1599 | YANG-2241 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTATGTTTCCTGGTACCAGCAATTCCCAGGAACAGCC |
| | | sequence | CCCAAACTCCTCATCTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGCTGACCGTCCTG |
| 1600 | YANG-2241 | VL domain | QSVLTQPPSVSAAPGQKVTISCSGSSSNIGNNYVSWYQQFPGTA |
| | | amino | PKLLIYDNNKRPSGIPDRFSGSKSGTSATLGITGLQTGDEADYY |
| | | acid | CGTWDSSLGAGVFGGGTKLTVL |
| | | sequence | |
| 1601 | YANG-2241 | LCDR1 | SSNIGNNY |
| 1602 | YANG-2241 | LCDR2 | DNN |
| 1603 | YANG-2241 | LCDR3 | GTWDSSLGAGV |
| 1604 | YANG-2242 | VH domain | CAGATGCAGCTGGTGCAGTCTGGGGCTGAGGTGAAGAAGCCTGG |
| | | nucleic | GGCCTCAGTGAAGATTTCCTGCAAGACATCTGGATACACCTTCA |
| | | acid | CCAGTTACTATATGGTCTGGGTGCGACAGGCCCCTGGACAAGGA |
| | | sequence | CTTGAATGGATGGGCATAATCAAACCTAGTGGTGGTAACACAAT |
| | | | CTACGCACAGAAATTCCAGGGCAGAGTCACCATGACCAGGGACA |
| | | | CGTCCACGAATACAGTCTTCATGGACCTGAGGAGTCTGAGATCT |
| | | | GAGGACACGGCCGTGTATTATTGTGCGAGAACTGGGGATAGGGC |
| | | | TTTTGATGTCTGGGGCCAAGGGACAGTGGTCACCGTCTCTTCA |
| 1605 | YANG-2242 | VH domain | QMQLVQSGAEVKKPGASVKISCKTSGYTFTSYYMVWVRQAPGQG |
| | | amino | LEWMGIIKPSGGNTIYAQKFQGRVTMTRDTSINTVFMDLRSLRS |
| | | acid | EDTAVYYCARTGDRAFDVWGQGTVVTVSS |
| | | sequence | |
| 1606 | YANG-2242 | HCDR1 | GYTFTSYY |
| 1607 | YANG-2242 | HCDR2 | IKPSGGNT |
| 1608 | YANG-2242 | HCDR3 | ARTGDRAFDV |
| 1609 | YANG-2242 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTTTGTATCCTGGTACCTGCAGTTCCCAGGAACAGCC |
| | | sequence | CCCAAACTCCTCATTTTTGACAATAATAAGCGACCCTCAGGGAT |
| | | | TCCTGACCGATTCTCTGGCTCCAAGTCTGGCACGTCAGCCACCC |
| | | | TGGGCATCTCCGGACTCCAGACTGGGGACGAGGCCGATTATTAC |
| | | | TGCGGAACATGGGATAGCAGCCTGGGTGCTGGGGTGTTCGGCGG |
| | | | AGGGACCCAACTGACCGTCCTA |
| 1610 | YANG-2242 | VL domain | QSVLTQPPSVSAAPGQKVTISCSGSSSNIGNNFVSWYLQFPGTA |
| | | amino | PKLLIFDNNKRPSGIPDRFSGSKSGTSATLGISGLQTGDEADYY |
| | | acid | CGTWDSSLGAGVFGGGTQLTVL |
| | | sequence | |
| 1611 | YANG-2242 | LCDR1 | SSNIGNNE |
| 1612 | YANG-2242 | LCDR2 | DNN |
| 1613 | YANG-2242 | LCDR3 | GTWDSSLGAGV |
| 1614 | YANG-2243 | VH domain | CAGATGCAGTTGGTGCAGTCTGGGGCTGAGGTGAAGAAGCCTGG |
| | | nucleic | GGCCTCAGTGAAGATTTCCTGCAAGACATCTGGATACACCTTCA |
| | | acid | CCAATTACTATATGGTCTGGGTGCGACAGGCCCCTGGACAAGGA |
| | | sequence | CTTGAGTGGATGGGCATAATCAAACCTAGTGGTGGTAACACGAT |
| | | | CTACGCACAGAAGTTCCAGGGCAGAGTCACCATGACCAGGGACA |
| | | | CGTCCACGAACACAGTCTATATGGACCTGAGCAGTCTGAGATCT |
| | | | GAGGACACGGCCGTGTATTATTGTGCGAGAACTGGGGATAGGGC |
| | | | TTTTGATGTCTGGGGCCACGGGACAATGGTCACCGTCTCTTCA |
| 1615 | YANG-2243 | VH domain | QMQLVQSGAEVKKPGASVKISCKTSGYTFTNYYMVWVRQAPGQG |
| | | amino | LEWMGIIKPSGGNTIYAQKFQGRVTMTRDTSTNTVYMDLSSLRS |
| | | acid | EDTAVYYCARTGDRAFDVWGHGTMVTVSS |
| | | sequence | |
| 1616 | YANG-2243 | HCDR1 | GYTFTNYY |
| 1617 | YANG-2243 | HCDR2 | IKPSGGNT |
| 1618 | YANG-2243 | HCDR3 | ARTGDRAFDV |
| 1619 | YANG-2243 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTATCTTTCCTGGTACCAGCAGTTCCCAGGAACAGCC |
| | | sequence | CCCAAACTCCTCATTTATGACAATAATAAACGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGGTGACCGTCCTA |
| 1620 | YANG-2243 | VL domain | QSVLTQPPSVSAAPGQKVTISCSGSSSNIGNNYLSWYQQFPGTA |
| | | amino | PKLLIYDNNKRPSGIPDRFSGSKSGTSATLGITGLQTGDEADYY |
| | | acid | CGTWDSSLGAGVFGGGTKVTVL |
| | | sequence | |
| 1621 | YANG-2243 | LCDR1 | SSNIGNNY |
| 1622 | YANG-2243 | LCDR2 | DNN |
| 1623 | YANG-2243 | LCDR3 | GTWDSSLGAGV |
| 1624 | YANG-2244 | VH domain | CAGATGCAATTGGTGCAGTCTGGGGCTGAGGTGAAGAAGCCTGG |
| | | nucleic | GACCTCAGTGAAGATTTCCTGCAAGACATCTGGATACACCTTCA |
| | | acid | CCAACTACTATATGGTCTGGGTGCGACAGGCCCCTGGACAAGGA |
| | | sequence | CTTGAGTGGATGGGCATAATCAAACCTAGTGGTGGTAACACGAT |
| | | | CTACGCACAGAAGTTCCAGGGCAGAGTCACCATGACCAGGGACA |
| | | | CGTCCACGAACACAGTCTATTTGGACCTGAGCAGTCTGAGATCT |
| | | | GAGGACACGGCCGTGTATTATTGTGCGAGAACTGGGGATAGGGC |
| | | | TTTTGATGTCTGGGGCCACGGGACAATGGTCACCGTCTCTTCA |
| 1625 | YANG-2244 | VH domain | QMQLVQSGAEVKKPGTSVKISCKTSGYTFTNYYMVWVRQAPGQG |
| | | amino | LEWMGIIKPSGGNTIYAQKFQGRVTMTRDTSTNTVYLDLSSLRS |
| | | acid | EDTAVYYCARTGDRAFDVWGHGTMVTVSS |
| | | sequence | |
| 1626 | YANG-2244 | HCDR1 | GYTFTNYY |
| 1627 | YANG-2244 | HCDR2 | IKPSGGNT |
| 1628 | YANG-2244 | HCDR3 | ARTGDRAFDV |
| 1629 | YANG-2244 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTATGTTTCCTGGTACCAGCAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCACGCTGACCGTCCTA |
| 1630 | YANG-2244 | VL domain | QSVLTQPPSVSAAPGQKVTISCSGSSSNIGNNYVSWYQQFPGTA |
| | | amino | PKLLIYDNNKRPSGIPDRESGSKSGTSATLGITGLQTGDEADYY |
| | | acid | CGTWDSSLGAGVFGGGTTLTVL |
| | | sequence | |
| 1631 | YANG-2244 | LCDR1 | SSNIGNNY |
| 1632 | YANG-2244 | LCDR2 | DNN |
| 1633 | YANG-2244 | LCDR3 | GTWDSSLGAGV |
| 1634 | YANG-2245 | VH domain | CAGATTCAGTTGGTGCAGTCTGGGGCTGAGGTGAAGAAGCCTGG |
| | | nucleic | GGCCTCAGTGAAGTTTTCCTGTAAGACATCTGGATACACCTTCA |
| | | acid | CCAGCTACTATATGGTCTGGGTGCGACAGGCCCCTGGACAAGGA |
| | | sequence | CTTGAGTGGATGGGCATAATCAAACCTAGTGGTGGTAACACAAT |
| | | | CTACGCACAGAAGTTCCAGGGCAGAGTCACCATGACCAGGGACA |
| | | | CGTCCACAAACACAGTCTACATGGACCTGAACAGTCTGAGATCT |
| | | | GAGGACACGGCCGTCTATTATTGTGCGAGAACTGGAGATAGGGC |
| | | | TTTTGATGTCTGGGGCCACGGGACAATGGTCACCGTGTCTTCA |
| 1635 | YANG-2245 | VH domain | QIQLVQSGAEVKKPGASVKFSCKTSGYTFTSYYMVWVRQAPGQG |
| | | amino | LEWMGIIKPSGGNTIYAQKFQGRVTMTRDTSTNTVYMDLNSLRS |
| | | acid | EDTAVYYCARTGDRAFDVWGHGTMVTVSS |
| | | sequence | |
| 1636 | YANG-2245 | HCDR1 | GYTFTSYY |
| 1637 | YANG-2245 | HCDR2 | IKPSGGNT |
| 1638 | YANG-2245 | HCDR3 | ARTGDRAFDV |
| 1639 | YANG-2245 | VL domain | CAGTCTGTGTTGACGCAGCCGCCCTCAGTGTCTGCGGCCCCAGG |
| | | nucleic | ACAGAAGGTCCCCATCTCCTGCCCTGGAAGCAGCTCCAACATTG |
| | | acid | GGAATAATTATGTATCCTGGTACCACCAGTTCCCAGGGACAACC |
| | | sequence | CCCAAACTCCTCATTTATGACAATAATAAGCGACCCTCAGGGAT |
| | | | TCCTGACCGATTCTCCGGCTCCAAGTCTGGCACGGCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAACTGACCGTCCTA |
| 1640 | YANG-2245 | VL domain | QSVLTQPPSVSAAPGQKVPISCPGSSSNIGNNYVSWYHQFPGTT |
| | | amino | PKLLIYDNNKRPSGIPDRFSGSKSGTAATLGITGLQTGDEADYY |
| | | acid | CGTWDSSLGAGVFGGGTKLTVL |
| | | sequence | |
| 1641 | YANG-2245 | LCDR1 | SSNIGNNY |
| 1642 | YANG-2245 | LCDR2 | DNN |
| 1643 | YANG-2245 | LCDR3 | GTWDSSLGAGV |
| 1644 | YANG-2246 | VH domain | CAGATGCAGTTGGTGCAGTCTGGGGCTGAGGTGAAGAAGCCTGG |
| | | nucleic | GGCCTCAGTGAAGATTTCCTGCAAGACATCTGGATACACCTTCA |
| | | acid | CCAACTACTATATGGTCTGGGTGCGACAGGCCCCGGGACAAGGA |
| | | sequence | CTTGAGTGGATGGGCATAATCAAACCTAGTGGCGGTGACACGAT |
| | | | CTACGCACAGAAGTTCCAGGGCAGAGTCACCATGACCAGGGACA |
| | | | CGTCCACGAACACAGTCTATATGGACCTGAGCAGTCTGAGATCT |
| | | | GAGGACACGGCCGTGTATTATTGTGCGAGAACTGGGAATAGGGC |
| | | | TTTTGATGTCTGGGGCCACGGGACAATGGTCACCGTCTCTTCA |
| 1645 | YANG-2246 | VH domain | QMQLVQSGAEVKKPGASVKISCKTSGYTFTNYYMVWVRQAPGQG |
| | | amino | LEWMGIIKPSGGDTIYAQKFQGRVTMTRDTSTNTVYMDLSSLRS |
| | | acid | EDTAVYYCARTGNRAFDVWGHGTMVTVSS |
| | | sequence | |
| 1646 | YANG-2246 | HCDR1 | GYTFTNYY |
| 1647 | YANG-2246 | HCDR2 | IKPSGGDT |
| 1648 | YANG-2246 | HCDR3 | ARTGNRAFDV |
| 1649 | YANG-2246 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTATGTTTCCTGGTACCAGCAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGCTGACCGTCCTA |
| 1650 | YANG-2246 | VL domain | QSVLTQPPSVSAAPGQKVTISCSGSSSNIGNNYVSWYQQFPGTA |
| | | amino | PKLLIYDNNKRPSGIPDRFSGSKSGTSATLGITGLQTGDEADYY |
| | | acid | CGTWDSSLGAGVFGGGTKLTVL |
| | | sequence | |
| 1651 | YANG-2246 | LCDR1 | SSNIGNNY |
| 1652 | YANG-2246 | LCDR2 | DNN |
| 1653 | YANG-2246 | LCDR3 | GTWDSSLGAGV |
| 1654 | YANG-2247 | VH domain | CAGATGCAGTTGGTGCAGTCTGGGGCTGAGGTGAAGAAGCCTGG |
| | | nucleic | GGCCTCAGTGAAGATTTCCTGCAAGACATCTGGATACACCTTCA |
| | | acid | CCGACTACTATATGGTCTGGGTGCGACAGGCCCCTGGACAAGGA |
| | | sequence | CTTGAGTGGATGGGCATGATCAAACCCAGTGGTGGTAACACGAT |
| | | | CTACGCACAGAAGTTCCAGGGCAGAGTCACCATGACCAGGGACA |
| | | | CGTCCACGAACACAGTCTATATGGACCTGAGCAGTCTGAGGTCT |
| | | | GAGGACACGGCCGTGTATTATTGCACAAGAACTGGGAATAGGGC |
| | | | TTTTGATGTCTGGGGCCACGGGACAATGGTCACCGTCTCTTCA |
| 1655 | YANG-2247 | VH domain | QMQLVQSGAEVKKPGASVKISCKTSGYTFTDYYMVWVRQAPGQG |
| | | amino | LEWMGMIKPSGGNTIYAQKFQGRVTMTRDTSTNTVYMDLSSLRS |
| | | acid | EDTAVYYCTRTGNRAFDVWGHGTMVTVSS |
| | | sequence | |
| 1656 | YANG-2247 | HCDR1 | GYTFTDYY |
| 1657 | YANG-2247 | HCDR2 | IKPSGGNT |
| 1658 | YANG-2247 | HCDR3 | TRTGNRAFDV |
| 1659 | YANG-2247 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | CGAATAATTATGTTTCCTGGTACCAGCAGTTCCCAGGAACAGCC |
| | | sequence | CCCAAACTCCTCATTTTTGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGCTGACCGTCCTA |
| 1660 | YANG-2247 | VL domain | QSVLTQPPSVSAAPGQKVTISCSGSSSNIANNYVSWYQQFPGTA |
| | | amino | PKLLIFDNNKRPSGIPDRFSGSKSGTSATLGITGLQTGDEADYY |
| | | acid | CGTWDSSLGAGVFGGGTKLTVL |
| | | sequence | |
| 1661 | YANG-2247 | LCDR1 | SSNIANNY |
| 1662 | YANG-2247 | LCDR2 | DNN |
| 1663 | YANG-2247 | LCDR3 | GTWDSSLGAGV |
| 1664 | YANG-2248 | VH domain | CAGATGCAATTGGTGCAATCTGGGGCTGAGGTGAAGAAGCCTGG |
| | | nucleic | GGCCTCAGTGAAGATTTCCTGCAAGACATCTGGATACACCTTCA |
| | | acid | CCAACTACTATATGGTCTGGGTGCGACAGGCCCCTGGCCAAGGA |
| | | sequence | CTTGAGTGGATGGGCATAATCAAACCTAGTGGTGGTAACACGAT |
| | | | CTACGCACAGAAGTTCCAGGACAGAGTCACCATGACCAGGGACA |
| | | | CGTCCACGAACACAGTCTATTTGGACCTGAGCAGTCTGAGATCT |
| | | | GAGGACACGGCCGTCTATTATTGTGCGAGAACTGGGGATAGGGC |
| | | | TTTTGATGTCTGGGGCCACGGGACAATGGTCACCGTCTCTTCA |
| 1665 | YANG-2248 | VH domain | QMQLVQSGAEVKKPGASVKISCKTSGYTFTNYYMVWVRQAPGQG |
| | | amino | LEWMGIIKPSGGNTIYAQKFQDRVTMTRDTSTNTVYLDLSSLRS |
| | | acid | EDTAVYYCARTGDRAFDVWGHGTMVTVSS |
| | | sequence | |
| 1666 | YANG-2248 | HCDR1 | GYTFTNYY |
| 1667 | YANG-2248 | HCDR2 | IKPSGGNT |
| 1668 | YANG-2248 | HCDR3 | ARTGDRAFDV |
| 1669 | YANG-2248 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTTTGTTTCCTGGTACCAGCAGTTCCCAGGAACAGCC |
| | | sequence | CCCAAACTCCTCATTTATGTCAATAATAAGCGACCCTCAGGGAT |
| | | | TCCTGACCGATTCTCTGGCTCCAGGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGCTGACCGTCCTA |
| 1670 | YANG-2248 | VL domain | QSVLTQPPSVSAAPGQKVTISCSGSSSNIGNNFVSWYQQFPGTA |
| | | amino | PKLLIYVNNKRPSGIPDRESGSRSGTSATLGITGLQTGDEADYY |
| | | acid | CGTWDSSLGAGVFGGGTKLTVL |
| | | sequence | |
| 1671 | YANG-2248 | LCDR1 | SSNIGNNF |
| 1672 | YANG-2248 | LCDR2 | VNN |
| 1673 | YANG-2248 | LCDR3 | GTWDSSLGAGV |
| 1674 | YANG-2249 | VH domain | CAGATGCAATTGGTGCAATCTGGGGCTGAGGTGAAGAAGCCTGG |
| | | nucleic | GGCCTCAGTGAAGATTTCCTGCAAGACATCTGGATACACCTTCA |
| | | acid | CCAAGTACTATATGGTCTGGGTGCGACAGGCCCCTGGACAAGGA |
| | | sequence | CTTGAGTGGATGGGCATAATCAAACCTAGTGGTGGTAACACGAT |
| | | | CTACGCACAGAAGTTCCAGGGCAGAGTCACCATGACCAGGGACA |
| | | | CGTCCACGAACACAGTCTATTTGGACCTGAGCAGTCTGAGATCT |
| | | | GAGGACACGGCCGTCTATTATTGTGCCAGAACTGGGGATAGGGC |
| | | | TTTTGATGTCTGGGGCCACGGGACAATGGTCACCGTCTCTTCA |
| 1675 | YANG-2249 | VH domain | QMQLVQSGAEVKKPGASVKISCKTSGYTFTKYYMVWVRQAPGQG |
| | | amino | LEWMGIIKPSGGNTIYAQKFQGRVTMTRDTSTNTVYLDLSSLRS |
| | | acid | EDTAVYYCARTGDRAFDVWGHGTMVTVSS |
| | | sequence | |
| 1676 | YANG-2249 | HCDR1 | GYTFTKYY |
| 1677 | YANG-2249 | HCDR2 | IKPSGGNT |
| 1678 | YANG-2249 | HCDR3 | ARTGDRAFDV |
| 1679 | YANG-2249 | VL domain | CAGTCTGTATTGACGCAGCCGCCCTCAGTGTCTGCGGCCCCAGG |
| | | nucleic | ACAGAAGGTCACCATCTCCTGCTCTGGAAGCAACTCCAACATTG |
| | | acid | GGAATAATTTTGTTTCCTGGTACCAGCAGTTCCCAGGAACAGCC |
| | | sequence | CCCAAACTCCTCATTTATGACAATAATAAACGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGCTGACCGTCCTA |
| 1680 | YANG-2249 | VL domain | QSVLTQPPSVSAAPGQKVTISCSGSNSNIGNNFVSWYQQFPGTA |
| | | amino | PKLLIYDNNKRPSGIPDRESGSKSGTSATLGITGLQTGDEADYY |
| | | acid | CGTWDSSLGAGVFGGGTKLTVL |
| | | sequence | |
| 1681 | YANG-2249 | LCDR1 | NSNIGNNF |
| 1682 | YANG-2249 | LCDR2 | DNN |
| 1683 | YANG-2249 | LCDR3 | GTWDSSLGAGV |
| 1684 | YANG-2250 | VH domain | CAGATACAGCTGGTGCAGTCTGGGGCTGAGGTGAAGAAGCCTGG |
| | | nucleic | GTCCTCAGTGAAGTTTTCCTGCAAGACATCTGGATACACCTTCA |
| | | acid | CCAACTACTATATGGTCTGGGTGCGACAGGCCCCTGGACAAGGA |
| | | sequence | CTTGAGTGGATGGGCATAATCAAACCTAGTGGTGGTAACACAAT |
| | | | CTACGCACAGAATTTCCAGGGCAGAGTCACCATGACCAGGGACA |
| | | | CGTCCACGAACACAGTCTACATGGACCTGAGGAGTCTGAGATCT |
| | | | GAGGACACGGCCGTCTATTATTGTGCGAGAACTGGTGATAGGGC |
| | | | TTTTGATGTCTGGGGCCAAGGGACAATGGTCACCGTCTCTTCA |
| 1685 | YANG-2250 | VH domain | QIQLVQSGAEVKKPGSSVKFSCKTSGYTFTNYYMVWVRQAPGQG |
| | | amino | LEWMGIIKPSGGNTIYAQNFQGRVTMTRDTSTNTVYMDLRSLRS |
| | | acid | EDTAVYYCARTGDRAFDVWGQGTMVTVSS |
| | | sequence | |
| 1686 | YANG-2250 | HCDR1 | GYTFTNYY |
| 1687 | YANG-2250 | HCDR2 | IKPSGGNT |
| 1688 | YANG-2250 | HCDR3 | ARTGDRAFDV |
| 1689 | YANG-2250 | VL domain | CAGTCTGTGTTGACGCAGCCGCCCTCAGTGTCTGCGACCCCAGG |
| | | nucleic | ACAGAAGGTCACCATCTCCTGCTCTGGAAGCAGCTCCAACATTG |
| | | acid | GGAATAATTTTGTATCCTGGTACCAGCAGTTCCCAGGAACAGCC |
| | | sequence | CCCCAACTCCTCATTTATGACAATAATAAGCGACCCTCAGGGAT |
| | | | TCCTGACCGATTCTCTGGCTCCCAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGCTGACCGTCCTA |
| 1690 | YANG-2250 | VL domain | QSVLTQPPSVSATPGQKVTISCSGSSSNIGNNFVSWYQQFPGTA |
| | | amino | PQLLIYDNNKRPSGIPDRFSGSQSGTSATLGITGLQTGDEADYY |
| | | acid | CGTWDSSLGAGVFGGGTKLTVL |
| | | sequence | |
| 1691 | YANG-2250 | LCDR1 | SSNIGNNE |
| 1692 | YANG-2250 | LCDR2 | DNN |
| 1693 | YANG-2250 | LCDR3 | GTWDSSLGAGV |
| 1694 | YANG-2251 | VH domain | CAGATTCAGTTGGTGCAGTCTGGGGCTGAGGTGAAGAAGCCTGG |
| | | nucleic | GGCCTCAGTGAAATTTTCCTGCAAGACATCTGGATACACCTTCA |
| | | acid | CCAGCTACTATATGGTCTGGGTGCGACAGGCCCCTGGACAAGGA |
| | | sequence | CTTGAGTGGATGGGCATAATCAAACCTAGTGGTGGTAACACAAT |
| | | | CTACGCACAGAAGTTCCAGGGCAGAGTCACCATGACCAGGGACA |
| | | | CGTCCACGAACACAGTCTTCATGGACCTGAACAGTCTGAGATCT |
| | | | GAGGACACGGCCGTCTATTATTGTGCGAGAACTGGAGATAGGGC |
| | | | TTTTGATGTCTGGGGCCACGGGACAATGGTCACCGTGTCTTCA |
| 1695 | YANG-2251 | VH domain | QIQLVQSGAEVKKPGASVKFSCKTSGYTFTSYYMVWVRQAPGQG |
| | | amino | LEWMGIIKPSGGNTIYAQKFQGRVTMTRDTSTNTVFMDLNSLRS |
| | | acid | EDTAVYYCARTGDRAFDVWGHGTMVTVSS |
| | | sequence | |
| 1696 | YANG-2251 | HCDR1 | GYTFTSYY |
| 1697 | YANG-2251 | HCDR2 | IKPSGGNT |
| 1698 | YANG-2251 | HCDR3 | ARTGDRAFDV |
| 1699 | YANG-2251 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGTTCCAACATTG |
| | | acid | GGAATAATTATGTATCCTGGTACCAGCAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAACTGACCGTCCTA |
| 1700 | YANG-2251 | VL domain | QSVLTQPPSVSAAPGQKVTISCSGSSSNIGNNYVSWYQQFPGTA |
| | | amino | PKLLIYDNNKRPSGIPDRFSGSKSGTSATLGITGLQTGDEADYY |
| | | acid | CGTWDSSLGAGVFGGGTKLTVL |
| | | sequence | |
| 1701 | YANG-2251 | LCDR1 | SSNIGNNY |
| 1702 | YANG-2251 | LCDR2 | DNN |
| 1703 | YANG-2251 | LCDR3 | GTWDSSLGAGV |
| 1704 | YANG-2252 | VH domain | CAGATACAGCTGGTGCAGTCTGGGGCTGAGGTGAAGAAGCCTGG |
| | | nucleic | GGCCTCAGTGAAGTTTTCCTGCAAGACATCTGGATACACCTTCA |
| | | acid | CCAACTACTATATGGTCTGGGTGCGACAGGCCCCTGGACAAGGA |
| | | sequence | CTTGAGTGGATGGGCATAATCAAACCTAGTGGTGGTAACACAAT |
| | | | CTACGCACAGAACTTCCAGGGCAGAGTCACCATGACCAGGGACA |
| | | | CGTCCACGAACACAGTCTACATGGACCTGAGGAGTCTGAGATCT |
| | | | GAGGACACGGCCGTGTATTATTGTGCGAGAACTGGTGATAGGGC |
| | | | TTTTGATGTCTGGGGCCAAGGGACAATGGTCACCGTCTCTTCA |
| 1705 | YANG-2252 | VH domain | QIQLVQSGAEVKKPGASVKFSCKTSGYTFTNYYMVWVRQAPGQG |
| | | amino | LEWMGIIKPSGGNTIYAQNFQGRVTMTRDTSTNTVYMDLRSLRS |
| | | acid | EDTAVYYCARTGDRAFDVWGQGTMVTVSS |
| | | sequence | |
| 1706 | YANG-2252 | HCDR1 | GYTFTNYY |
| 1707 | YANG-2252 | HCDR2 | IKPSGGNT |
| 1708 | YANG-2252 | HCDR3 | ARTGDRAFDV |
| 1709 | YANG-2252 | VL domain | CAGTCTGTGTTGACGCAGTCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTTTGTATCCTGGTACCAGCAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGCTGACCGTCCTA |
| 1710 | YANG-2252 | VL domain | QSVLTQSPSVSAAPGQKVTISCSGSSSNIGNNFVSWYQQFPGTA |
| | | amino | PKLLIYDNNKRPSGIPDRFSGSKSGTSATLGITGLQTGDEADYY |
| | | acid | CGTWDSSLGAGVFGGGTKLTVL |
| | | sequence | |
| 1711 | YANG-2252 | LCDR1 | SSNIGNNE |
| 1712 | YANG-2252 | LCDR2 | DNN |
| 1713 | YANG-2252 | LCDR3 | GTWDSSLGAGV |
| 1714 | YANG-2253 | VH domain | CAGATGCAATTGGTGCAGTCTGGGGCTGAGGTGAAGAAGCCTGG |
| | | nucleic | GGCCTCACTGAGGATTTCCTGCAAGACATCTGGATACACCTTCA |
| | | acid | CCAACTACTATATGGTCTGGGTGCGACAGGCCCCTGGACAAGGA |
| | | sequence | CTTGAGTGGATGGGCATAATCAAACCTAGTGGTGGTAACACGAT |
| | | | CTACGCACAGAAGTTCCAGGGCAGAGTCACCTTGACCAGGGACA |
| | | | CGTCCACGAACACAGTCTATTTGGACCTGAACAGTCTGAGATCT |
| | | | GAGGACACGGCCATGTATTATTGTGCGAGAACTGGGGATAGGGC |
| | | | CTTTGATGTCTGGGGCCACGGGACAATGGTCACCGTCTCTTCA |
| 1715 | YANG-2253 | VH domain | QMQLVQSGAEVKKPGASLRISCKTSGYTFTNYYMVWVRQAPGQG |
| | | amino | LEWMGIIKPSGGNTIYAQKFQGRVTLTRDTSTNTVYLDLNSLRS |
| | | acid | EDTAMYYCARTGDRAFDVWGHGTMVTVSS |
| | | sequence | |
| 1716 | YANG-2253 | HCDR1 | GYTFTNYY |
| 1717 | YANG-2253 | HCDR2 | IKPSGGNT |
| 1718 | YANG-2253 | HCDR3 | ARTGDRAFDV |
| 1719 | YANG-2253 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTATGTTTCCTGGTACCAACAGTTCCCAGGAACAGCC |
| | | sequence | CCCAAACTCCTCATTTATGACAATAATAAGCGACCCTCAGGGAT |
| | | | TCCTGACCGAGTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGCTGACCGTCCTA |
| 1720 | YANG-2253 | VL domain | QSVLTQPPSVSAAPGQKVTISCSGSSSNIGNNYVSWYQQFPGTA |
| | | amino | PKLLIYDNNKRPSGIPDRVSGSKSGTSATLGITGLQTGDEADYY |
| | | acid | CGTWDSSLGAGVFGGGTKLTVL |
| | | sequence | |
| 1721 | YANG-2253 | LCDR1 | SSNIGNNY |
| 1722 | YANG-2253 | LCDR2 | DNN |
| 1723 | YANG-2253 | LCDR3 | GTWDSSLGAGV |
| 1724 | YANG-2254 | VH domain | CAGATGCAGTTGGTGCAGTCTGGGGCTGAGGTGAAGAAGCCTGG |
| | | nucleic | GGCCTCAGTGAAATTTTCCTGCAAGACATCTGGATACACCTTCA |
| | | acid | CCAACTACTATATGGTCTGGGTGCGACAGGCCCCTGGACAAGGA |
| | | sequence | CTTGAGTGGATGGGCATAATCAAACCTAGTGGTGGTAACACAAT |
| | | | CTACGCACAGAAGTTCCAGGGCAGAGTCACCATGACCAGGGACA |
| | | | CGTCCACGAACACAGTCTACATGGACCTGAACAGTCTGACATCT |
| | | | GAGGACACGGCCGTATATTATTGTGCGAGAACTGGGAATAGGGC |
| | | | TTTTGATGTCTGGGGCCACGGGACAATGGTCACCGTCTCTTCA |
| 1725 | YANG-2254 | VH domain | QMQLVQSGAEVKKPGASVKFSCKTSGYTFTNYYMVWVRQAPGQG |
| | | amino | LEWMGIIKPSGGNTIYAQKFQGRVTMTRDTSTNTVYMDLNSLTS |
| | | acid | EDTAVYYCARTGNRAFDVWGHGTMVTVSS |
| | | sequence | |
| 1726 | YANG-2254 | HCDR1 | GYTFTNYY |
| 1727 | YANG-2254 | HCDR2 | IKPSGGNT |
| 1728 | YANG-2254 | HCDR3 | ARTGNRAFDV |
| 1729 | YANG-2254 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAATATTG |
| | | acid | GGAATAATTTTGTTTCCTGGTACCAGCAGTTTCCAGGAACAGCC |
| | | sequence | CCCAAACTCCTCATTTATGACAATTCTAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAACTGACCGTCCTA |
| 1730 | YANG-2254 | VL domain | QSVLTQPPSVSAAPGQKVTISCSGSSSNIGNNFVSWYQQFPGTA |
| | | amino | PKLLIYDNSKRPSGIPDRFSGSKSGTSATLGITGLQTGDEADYY |
| | | acid | CGTWDSSLGAGVFGGGTKLTVL |
| | | sequence | |
| 1731 | YANG-2254 | LCDR1 | SSNIGNNF |
| 1732 | YANG-2254 | LCDR2 | DNS |
| 1733 | YANG-2254 | LCDR3 | GTWDSSLGAGV |
| 1734 | YANG-2255 | VH domain | CAGATGCAATTGGTGCAATCTGGGGCTGAGGTGAAGAAGCCTGG |
| | | nucleic | GGCCTCAGTGAAGATTTCCTGCAAGACATCTGGATACACCTTCA |
| | | acid | CCAACTACTATATGGTCTGGGTGCGACAGGCCCCTGGACAAGGA |
| | | sequence | CTTGAGTGGATGGGCATAATCAAACCTAGTGGTGGTAACACGAT |
| | | | CTACACACAGAAGTTCCAGGGCAGAGTCACCATGACCAGGGACA |
| | | | CGTCCACGAACACAGTCTATTTGGACCTGAGCAGTCTGAGATCT |
| | | | GACGACACGGCCGTCTATTATTGTGCGAGAACTGGGGATAGGGC |
| | | | TTTTGATGTCTGGGGCCACGGGACAATGGTCACCGTCTCTTCA |
| 1735 | YANG-2255 | VH domain | QMQLVQSGAEVKKPGASVKISCKTSGYTFTNYYMVWVRQAPGQG |
| | | amino | LEWMGIIKPSGGNTIYTQKFQGRVTMTRDTSTNTVYLDLSSLRS |
| | | acid | DDTAVYYCARTGDRAFDVWGHGTMVTVSS |
| | | sequence | |
| 1736 | YANG-2255 | HCDR1 | GYTFTNYY |
| 1737 | YANG-2255 | HCDR2 | IKPSGGNT |
| 1738 | YANG-2255 | HCDR3 | ARTGDRAFDV |
| 1739 | YANG-2255 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTTTGTTTCCTGGTACCAGCAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTACTGGGGTGTTCGGCGG |
| | | | AGGGACCAAGCTGACCGTCCTA |
| 1740 | YANG-2255 | VL domain | QSVLTQPPSVSAAPGQKVTISCSGSSSNIGNNFVSWYQQFPGTA |
| | | amino | PKLLIYDNNKRPSGIPDRFSGSKSGTSATLGITGLQTGDEADYY |
| | | acid | CGTWDSSLGTGVFGGGTKLTVL |
| | | sequence | |
| 1741 | YANG-2255 | LCDR1 | SSNIGNNE |
| 1742 | YANG-2255 | LCDR2 | DNN |
| 1743 | YANG-2255 | LCDR3 | GTWDSSLGTGV |
| 1744 | YANG-2256 | VH domain | CAGATACAGTTGGTGCAGTCTGGGGCTGAGGTGAAGAAGCCTGG |
| | | nucleic | GGCCTCAGTGAAGCTTTCCTGCAAGACATCTGGATACACCTTCA |
| | | acid | CCAGCTACTATATGGTCTGGGTGCGACAGGCCCCTGGACAAGGA |
| | | sequence | CTTGAGTGGATGGGCATAATCAAACCTAGTGGTGGTAACACAAT |
| | | | CTACGCACAGCAGTTCCAGGGCAGAGCCACCATGACCAGGGACA |
| | | | CGTCCACGAACACAGTCTACATGGACCTGAGCAGTCTGAGATCT |
| | | | GAGGACACGGCCGTTTATTATTGTGCGAGAACTGGGGATAGGGC |
| | | | TTTTGATGTCTGGGGCCACGGGACACTGGTCACCGTCTCTTCA |
| 1745 | YANG-2256 | VH domain | QIQLVQSGAEVKKPGASVKLSCKTSGYTFTSYYMVWVRQAPGQG |
| | | amino | LEWMGIIKPSGGNTIYAQQFQGRATMTRDTSTNTVYMDLSSLRS |
| | | acid | EDTAVYYCARTGDRAFDVWGHGTLVTVSS |
| | | sequence | |
| 1746 | YANG-2256 | HCDR1 | GYTFTSYY |
| 1747 | YANG-2256 | HCDR2 | IKPSGGNT |
| 1748 | YANG-2256 | HCDR3 | ARTGDRAFDV |
| 1749 | YANG-2256 | VL domain | CAGTCTGTGTTGACGCAGCCGCCCTCAGTGTCTGCGGCCCCAGG |
| | | nucleic | ACAGAAGATCACCATCTCCTGCTCTGGAAGCAGCTCCAACATTG |
| | | acid | GAAATAATTTTGTATCCTGGTACCAGCAGTTCCCAGGAACAGCC |
| | | sequence | CCCAAACTCCTCATTTATGACAATAATAAGCGACCCTCAGGGAC |
| | | | TCCTGACCGATTCTCTGGCTCCAAGTCTGGCGCGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGCTGACCGTCCTG |
| 1750 | YANG-2256 | VL domain | QSVLTQPPSVSAAPGQKITISCSGSSSNIGNNFVSWYQQFPGTA |
| | | amino | PKLLIYDNNKRPSGTPDRFSGSKSGASATLGITGLQTGDEADYY |
| | | acid | CGTWDSSLGAGVFGGGTKLTVL |
| | | sequence | |
| 1751 | YANG-2256 | LCDR1 | SSNIGNNE |
| 1752 | YANG-2256 | LCDR2 | DNN |
| 1753 | YANG-2256 | LCDR3 | GTWDSSLGAGV |
| 1754 | YANG-2257 | VH domain | CAGATACAGTTGGTGCAGTCTGGGGCTGAGGTGAAGGAGCCTGG |
| | | nucleic | GGCCTCAGTGAAGTTTTCCTGCAAGACATCTGGATACACCTTCA |
| | | acid | CCAGCTACTATATGGTCTGGGTGCGACAGGCCCCTGGACAAGGA |
| | | sequence | CTTGAGTGGATGGGCATAATCAAATCTAGTGGTGGTGACACAAT |
| | | | CTACGCACAGAAGTTCCAGGGCCGAGTCACCATGACCAGGGACA |
| | | | CGTCCACGCACACAGTCTACATGGACCTGAACAGTCTGACATAT |
| | | | GAAGACACGGCCGTATATTATTGTGCGAGAAGTGGGAATAGGGC |
| | | | TTTTGATGTCTGGGGCCATGGGACAATGGTCACCGTCTCTTCA |
| 1755 | YANG-2257 | VH domain | QIQLVQSGAEVKEPGASVKFSCKTSGYTFTSYYMVWVRQAPGQG |
| | | amino | LEWMGIIKSSGGDTIYAQKFQGRVTMTRDTSTHTVYMDLNSLTY |
| | | acid | EDTAVYYCARSGNRAFDVWGHGTMVTVSS |
| | | sequence | |
| 1756 | YANG-2257 | HCDR1 | GYTFTSYY |
| 1757 | YANG-2257 | HCDR2 | IKSSGGDT |
| 1758 | YANG-2257 | HCDR3 | ARSGNRAFDV |
| 1759 | YANG-2257 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTTTGTATCCTGGTACCAACAGTTCCCAGGAACAGCC |
| | | sequence | CCCAAACTCCTCATTTATGACAATACTAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGCTGACCGTCCTA |
| 1760 | YANG-2257 | VL domain | QSVLTQPPSVSAAPGQKVTISCSGSSSNIGNNFVSWYQQFPGTA |
| | | amino | PKLLIYDNTKRPSGIPDRFSGSKSGTSATLGITGLQTGDEADYY |
| | | acid | CGTWDSSLGAGVFGGGTKLTVL |
| | | sequence | |
| 1761 | YANG-2257 | LCDR1 | SSNIGNNE |
| 1762 | YANG-2257 | LCDR2 | DNT |
| 1763 | YANG-2257 | LCDR3 | GTWDSSLGAGV |
| 1764 | YANG-2258 | VH domain | CAGATGCAATTGGTGCAGTCTGGGGCTGAGGTGAAGAAGCCTGG |
| | | nucleic | GGCCTCAGTGAAGATTTCCTGCAAGACATCTGGATACACCTTCA |
| | | acid | CCAACTACTATATGGTCTGGGTGCGACAGGCCCCTGGACAAGGA |
| | | sequence | CTTGAGTGGATGGGCATAATCAAACCTAGTGGCGGTAACACGAT |
| | | | CTACGCACAGAAGTTCCAGGGCAGAGTCACCATGACCCGGGACA |
| | | | CGTCCACGAACACAGTCTATTTGGACCTGAGCAGTCTGAGATCT |
| | | | GAGGATACGGCCGTGTATTATTGTGCGAGAACTGGGGATAGGGC |
| | | | TTTTGATGTCTGGGGCCACGGGACAATGGTCACCGTCTCTTCA |
| 1765 | YANG-2258 | VH domain | QMQLVQSGAEVKKPGASVKISCKTSGYTFTNYYMVWVRQAPGQG |
| | | amino | LEWMGIIKPSGGNTIYAQKFQGRVTMTRDTSTNTVYLDLSSLRS |
| | | acid | EDTAVYYCARTGDRAFDVWGHGTMVTVSS |
| | | sequence | |
| 1766 | YANG-2258 | HCDR1 | GYTFTNYY |
| 1767 | YANG-2258 | HCDR2 | IKPSGGNT |
| 1768 | YANG-2258 | HCDR3 | ARTGDRAFDV |
| 1769 | YANG-2258 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTATGTTTCCTGGTACCAGCAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGTAACATGGGATAGCAGCCTGGGTGCTGGGGTGTTCGGCGG |
| | | | AGGGACCAAGCTGACCGTCCTA |
| 1770 | YANG-2258 | VL domain | QSVLTQPPSVSAAPGQKVTISCSGSSSNIGNNYVSWYQQFPGTA |
| | | amino | PKLLIYDNNKRPSGIPDRFSGSKSGTSATLGITGLQTGDEADYY |
| | | acid | CVTWDSSLGAGVFGGGTKLTVL |
| | | sequence | |
| 1771 | YANG-2258 | LCDR1 | SSNIGNNY |
| 1772 | YANG-2258 | LCDR2 | DNN |
| 1773 | YANG-2258 | LCDR3 | VTWDSSLGAGV |
| 1774 | YANG-2259 | VH domain | CAGATGCAATTGGTGCAATCTGGGGCTGAGGTGAAGAAGCCTGG |
| | | nucleic | GGCCTCAGTGAAGATTTCCTGCAAGACATCTGGATACACCTTCA |
| | | acid | CCAACTACTATATGGTCTGGGTGCGACAGGCCCCTGGACAAGGA |
| | | sequence | CTTGAGTGGATGGGCATAATCAAACCTAGTGGTGGTAACACGAT |
| | | | CTACGCACAGAAGTTCCAGGGCAGAGTCACCATGACCAGGGACA |
| | | | CGTCCACGAATAAAGTCTATTTGGACCTGAGCAGTCTGAGATCT |
| | | | GAGGACACGGCCGTCTATTATTGTGCCAGAACTGGGGATAGGGC |
| | | | TTTTGATGTCTGGGGCCAGGGGACAATGGTCACCGTCTCTTCA |
| 1775 | YANG-2259 | VH domain | QMQLVQSGAEVKKPGASVKISCKTSGYTFTNYYMVWVRQAPGQG |
| | | amino | LEWMGIIKPSGGNTIYAQKFQGRVTMTRDTSTNKVYLDLSSLRS |
| | | acid | EDTAVYYCARTGDRAFDVWGQGTMVTVSS |
| | | sequence | |
| 1776 | YANG-2259 | HCDR1 | GYTFTNYY |
| 1777 | YANG-2259 | HCDR2 | IKPSGGNT |
| 1778 | YANG-2259 | HCDR3 | ARTGDRAFDV |
| 1779 | YANG-2259 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTTTGTTTCCTGGTACCAGCAGTTCCCAGGAACAGCC |
| | | sequence | CCCAAACTCCTCATTTATGACAATAATAAACGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGCTGACCGTCCTA |
| 1780 | YANG-2259 | VL domain | QSVLTQPPSVSAAPGQKVTISCSGSSSNIGNNFVSWYQQFPGTA |
| | | amino | PKLLIYDNNKRPSGIPDRFSGSKSGTSATLGITGLQTGDEADYY |
| | | acid | CGTWDSSLGAGVFGGGTKLTVL |
| | | sequence | |
| 1781 | YANG-2259 | LCDR1 | SSNIGNNF |
| 1782 | YANG-2259 | LCDR2 | DNN |
| 1783 | YANG-2259 | LCDR3 | GTWDSSLGAGV |
| 1784 | YANG-2260 | VH domain | CAGATGCAGTTGGTGCAGTCTGGGGCTGAGGTGAAGAAGCCTGG |
| | | nucleic | GGCCTCAGTGAAGATTTCCTGCAAGACATCTGGATACACCTTCA |
| | | acid | TCAACTACTATATGGTCTGGGTGCGACAGGCCCCTGGACAAGGA |
| | | sequence | CTTGAGTGGATGGGCATAATCAAACCTAGTGGTGGTAACACGAT |
| | | | CTACGCACAGAAGTTCCAGGGCAGAGTCACCATGACCAGGGACA |
| | | | CGTCCACGAACACAGTCTATATGGACCTGAGCAGTCTGAGATCT |
| | | | GAGGACACGGCCGTGTATTATTGTGCGAGAATTGGGGATAGGGC |
| | | | TTTTGATGTCTGGGGCCACGGGACAATGGTCACCGTCTCTTCA |
| 1785 | YANG-2260 | VH domain | QMQLVQSGAEVKKPGASVKISCKTSGYTFINYYMVWVRQAPGQG |
| | | amino | LEWMGIIKPSGGNTIYAQKFQGRVTMTRDTSTNTVYMDLSSLRS |
| | | acid | EDTAVYYCARIGDRAFDVWGHGTMVTVSS |
| | | sequence | |
| 1786 | YANG-2260 | HCDR1 | GYTFINYY |
| 1787 | YANG-2260 | HCDR2 | IKPSGGNT |
| 1788 | YANG-2260 | HCDR3 | ARIGDRAFDV |
| 1789 | YANG-2260 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAACTTTGTTTCCTGGTACCAGCAGTTCCCAGGAACAGCC |
| | | sequence | CCCAAGCTCCTCATTTATGACAATCATAAGCGACCCTCAGGGAT |
| | | | TCCTGACCGATTCTCTGCCTCCAAGTCTGGCACGTCAGCCACCC |
| | | | TGGGCATCACCGGACTCCAGACTGGGGACGAGGCCGATTATTAC |
| | | | TGCGGAACATGGGATAGCGGCCTGGGTGCTGGGGTGTTCGGCGG |
| | | | AGGGACCAAGCTGACCGTCCTA |
| 1790 | YANG-2260 | VL domain | QSVLTQPPSVSAAPGQKVTISCSGSSSNIGNNFVSWYQQFPGTA |
| | | amino | PKLLIYDNHKRPSGIPDRFSASKSGTSATLGITGLQTGDEADYY |
| | | acid | CGTWDSGLGAGVFGGGTKLTVL |
| | | sequence | |
| 1791 | YANG-2260 | LCDR1 | SSNIGNNE |
| 1792 | YANG-2260 | LCDR2 | DNH |
| 1793 | YANG-2260 | LCDR3 | GTWDSGLGAGV |
| 1794 | YANG-2261 | VH domain | CAGATTCAATTGGTGCAGTCTGGGGCTGAGGTGAAGAAGCCTGG |
| | | nucleic | GGCCTCAGTGAAGATTTCCTGCAAGACATCTGGATACACCTTCA |
| | | acid | CCAACTACTATATGGTCTGGGTGCGACAGGCCCCTGGACAAGGA |
| | | sequence | CTTGAGTGGATGGGCATAATCAAACCTAGTAATGGTAACACGAT |
| | | | CTACGCACAGAAGTTCCAGGGCAGAGTCACCATGACCAGGGACA |
| | | | CGTCCACGAATATAGTCTATTTGGACCTGAGCAGTCTGAGATCT |
| | | | GAGGACACGGCCGTGTATTATTGTGCGAGAACTGGGGATAGGGC |
| | | | TTTTGATGTCTGGGGCCACGGGACAATGGTCACCGTCTCTTCA |
| 1795 | YANG-2261 | VH domain | QIQLVQSGAEVKKPGASVKISCKTSGYTFTNYYMVWVRQAPGQG |
| | | amino | LEWMGIIKPSNGNTIYAQKFQGRVTMTRDTSTNIVYLDLSSLRS |
| | | acid | EDTAVYYCARTGDRAFDVWGHGTMVTVSS |
| | | sequence | |
| 1796 | YANG-2261 | HCDR1 | GYTFTNYY |
| 1797 | YANG-2261 | HCDR2 | IKPSNGNT |
| 1798 | YANG-2261 | HCDR3 | ARTGDRAFDV |
| 1799 | YANG-2261 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAATATTG |
| | | acid | GGAATAATTATGTTTCCTGGTACCAGCAGTTCCCAGGAACAGTC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGAATATTAC |
| | | | TGCGGAACATGGGATAGCAGCCTGGGTGCTGGGGTGTTCGGCGG |
| | | | AGGGACCAAGCTGACCGTCCTA |
| 1800 | YANG-2261 | VL domain | QSVLTQPPSVSAAPGQKVTISCSGSSSNIGNNYVSWYQQFPGTV |
| | | amino | PKLLIYDNNKRPSGIPDRESGSKSGTSATLGITGLQTGDEAEYY |
| | | acid | CGTWDSSLGAGVFGGGTKLTVL |
| | | sequence | |
| 1801 | YANG-2261 | LCDR1 | SSNIGNNY |
| 1802 | YANG-2261 | LCDR2 | DNN |
| 1803 | YANG-2261 | LCDR3 | GTWDSSLGAGV |
| 1804 | YANG-2262 | VH domain | CAGATACAGCTGGTGCAGTCTGGGGCTGAGGTGAAGAAGCCTGG |
| | | nucleic | GGCCTCAGTGAGGTTTTCCTGCAAGACATCTGGATACACCTTCA |
| | | acid | CCAACTACTACATGGTCTGGGTGCGACAGGCCCCTGGACAAGGA |
| | | sequence | CTTGAGTGGATGGGCATAATCAAACCTAGTGGTGGTGACACAAT |
| | | | CTACGCACAGAACTTCCAGGGCAGAGTCACCATGACCAGGGACA |
| | | | CGTCCACGAACTCAGTCTATATGGACCTGAGGAGTCTGAGATCT |
| | | | GAGGACACGGCCGTGTATTATTGTGCGAGAACTGGTGATAGGGC |
| | | | TTTTGATGTCTGGGGCCAAGGGACAATGGTCACCGTCTCTTCA |
| 1805 | YANG-2262 | VH domain | QIQLVQSGAEVKKPGASVRFSCKTSGYTFTNYYMVWVRQAPGQG |
| | | amino | LEWMGIIKPSGGDTIYAQNFQGRVTMTRDTSTNSVYMDLRSLRS |
| | | acid | EDTAVYYCARTGDRAFDVWGQGTMVTVSS |
| | | sequence | |
| 1806 | YANG-2262 | HCDR1 | GYTFTNYY |
| 1807 | YANG-2262 | HCDR2 | IKPSGGDT |
| 1808 | YANG-2262 | HCDR3 | ARTGDRAFDV |
| 1809 | YANG-2262 | VL domain | CAGTCTGTGTTGACGCAGCCGCCCTCAGTGTCTGCGGCCCCAGG |
| | | nucleic | ACAGAAGGTCACCATCTCCTGCTCTGGACGCAGCTCCAACATTG |
| | | acid | GGAATAATTTTGTATCCTGGTACCAGCAGTTCCCAGGAACAGCC |
| | | sequence | CCCAAACTCCTCATTTATGACAATAATAAACGACCCTCAGGGAT |
| | | | TCCTGACCGATTCTCTGGCTCCAAGTCTGGCACGTCAGCCACCC |
| | | | TGGGCATCACCGGGCTCCAGACTGGGGACGAGGCCGATTATTAC |
| | | | TGCGGAACATGGGATAGCAGCCTGGGTGCTGGGGTGTTCGGCGG |
| | | | AGGGACCAAGCTGACCGTCCTA |
| 1810 | YANG-2262 | VL domain | QSVLTQPPSVSAAPGQKVTISCSGRSSNIGNNFVSWYQQFPGTA |
| | | amino | PKLLIYDNNKRPSGIPDRFSGSKSGTSATLGITGLQTGDEADYY |
| | | acid | CGTWDSSLGAGVFGGGTKLTVL |
| | | sequence | |
| 1811 | YANG-2262 | LCDR1 | SSNIGNNE |
| 1812 | YANG-2262 | LCDR2 | DNN |
| 1813 | YANG-2262 | LCDR3 | GTWDSSLGAGV |
| 1814 | YANG-2263 | VH domain | CAGATGCAATTGGTGCAGTCTGGGGCTGAGGTGAAGAAGCCTGG |
| | | nucleic | GGCCTCAGTGAAGATTTCCTGCAAGACATCTGGATTCACCTTCG |
| | | acid | CCAACTACTATGTGGTCTGGGTGCGACAGGCCCCTGGACAAGGA |
| | | sequence | CTTGAGTGGATGGGCATAATCAAACCTAGTGGTGGTAACACGAT |
| | | | CTACGCACAGAAGTTCCAGGGCAGAGTCACCATGACCAGGGACA |
| | | | CGTCCACGAACACAGTCTATTTGGACTTGAGCAGTCTGAGATCT |
| | | | GAGGACACGGCCGTGTATTATTGTGCGAGAACTGGTGATAGGGC |
| | | | TTTTGATGTCTGGGGCCACGGGACAGTGGTCACCGTCTCTTCA |
| 1815 | YANG-2263 | VH domain | QMQLVQSGAEVKKPGASVKISCKTSGFTFANYYVVWVRQAPGQG |
| | | amino | LEWMGIIKPSGGNTIYAQKFQGRVTMTRDTSTNTVYLDLSSLRS |
| | | acid | EDTAVYYCARTGDRAFDVWGHGTVVTVSS |
| | | sequence | |
| 1816 | YANG-2263 | HCDR1 | GFTFANYY |
| 1817 | YANG-2263 | HCDR2 | IKPSGGNT |
| 1818 | YANG-2263 | HCDR3 | ARTGDRAFDV |
| 1819 | YANG-2263 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTATGTTTCCTGGTACCAGCAGTTCCCAGGAACAGCC |
| | | sequence | CCCAAACTCCTCATCTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGCTGACCGTCCTA |
| 1820 | YANG-2263 | VL domain | QSVLTQPPSVSAAPGQKVTISCSGSSSNIGNNYVSWYQQFPGTA |
| | | amino | PKLLIYDNNKRPSGIPDRFSGSKSGTSATLGITGLQTGDEADYY |
| | | acid | CGTWDSSLGAGVFGGGTKLTVL |
| | | sequence | |
| 1821 | YANG-2263 | LCDR1 | SSNIGNNY |
| 1822 | YANG-2263 | LCDR2 | DNN |
| 1823 | YANG-2263 | LCDR3 | GTWDSSLGAGV |
| 1824 | YANG-2264 | VH domain | CAGATACAGTTGGTGCAGTCTGGGGCTGAGGTGAAGAAGCCTGG |
| | | nucleic | GGCCTCAGTGAAGCTTTCCTGCAAGACATCTGGATACACCTTCA |
| | | acid | CCAGCTACTACATGGTCTGGGTGCGACAGGCCCCTGGACAAGGG |
| | | sequence | CTTGAATGGATGGGCATAATCAAACCTAGTGGTGGTAACACAAT |
| | | | CTACGCACAGAAGTTCCAGGGCAGAGCCACCATGACCAGGGACA |
| | | | CGTCCACGAACACAGTCTACATGGACCTGAGCAGTCTGAGATCT |
| | | | GAAGACACGGCCGTGTATTATTGTGCGAGAACTGGGGATAGGGC |
| | | | TTTTGATGTCTGGGGCCACGGGACACTGGTCACCGTCTCTTCA |
| 1825 | YANG-2264 | VH domain | QIQLVQSGAEVKKPGASVKLSCKTSGYTFTSYYMVWVRQAPGQG |
| | | amino | LEWMGIIKPSGGNTIYAQKFQGRATMTRDTSTNTVYMDLSSLRS |
| | | acid | EDTAVYYCARTGDRAFDVWGHGTLVTVSS |
| | | sequence | |
| 1826 | YANG-2264 | HCDR1 | GYTFTSYY |
| 1827 | YANG-2264 | HCDR2 | IKPSGGNT |
| 1828 | YANG-2264 | HCDR3 | ARTGDRAFDV |
| 1829 | YANG-2264 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTATGTATCCTGGTACCAGCAGATCCCAGGAACAGCC |
| | | sequence | CCCAAACTCCTCATTTATGACAATAATAAGCGACCCTCAGGGAC |
| | | | TCCTGCCCGATCCTCTGGCTCCAAGTCTGGCGCGTCAGCCACCC |
| | | | TGGGCATCACCGGACTCCAGCCTGGGGACGAGGCCGATTATTAC |
| | | | TGCGGAACATGGGATAGCAGCCTGGGTGCTGGGGTGTTCGGCGG |
| | | | AGGGACCAAGCTGACCGTCCTG |
| 1830 | YANG-2264 | VL domain | QSVLTQPPSVSAAPGQKVTISCSGSSSNIGNNYVSWYQQIPGTA |
| | | amino | PKLLIYDNNKRPSGTPARSSGSKSGASATLGITGLQPGDEADYY |
| | | acid | CGTWDSSLGAGVFGGGTKLTVL |
| | | sequence | |
| 1831 | YANG-2264 | LCDR1 | SSNIGNNY |
| 1832 | YANG-2264 | LCDR2 | DNN |
| 1833 | YANG-2264 | LCDR3 | GTWDSSLGAGV |
| 1834 | YANG-2265 | VH domain | CAGATACAGCTGGTGCAGTCTGGGGCTGAGGTGAAGAAGCCTGG |
| | | nucleic | GGCCTCAGTGAACTTTTCCTGCAAGACATCTGGATACACCTTCA |
| | | acid | CCAACTACTATATGGTCTGGGTGCGACAGGCCCCTGGACAAGGA |
| | | sequence | CTTGAGTGGATGGGCATAATCAAACCTAGTGGTGGTAACACAAT |
| | | | CTACGCACAGAAGTTCCAGGGCAGAGTCACCATGACCAGGGACA |
| | | | CGTCCACGAACACAGTCTACATGGACCTGAGGAGTCTGCGATCT |
| | | | GAGGACACGGCCGTGTATTATTGTGCGAGAACTGGGGATAGGGC |
| | | | TTTTGATGTCTGGGGCCAAGGGACAATGGTCACCGTCTCTTCA |
| 1835 | YANG-2265 | VH domain | QIQLVQSGAEVKKPGASVNFSCKTSGYTFTNYYMVWVRQAPGQG |
| | | amino | LEWMGIIKPSGGNTIYAQKFQGRVTMTRDTSTNTVYMDLRSLRS |
| | | acid | EDTAVYYCARTGDRAFDVWGQGTMVTVSS |
| | | sequence | |
| 1836 | YANG-2265 | HCDR1 | GYTFTNYY |
| 1837 | YANG-2265 | HCDR2 | IKPSGGNT |
| 1838 | YANG-2265 | HCDR3 | ARTGDRAFDV |
| 1839 | YANG-2265 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTTTGTATCCTGGTACCAGCAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGCTGACCGTCCTA |
| 1840 | YANG-2265 | VL domain | QSVLTQPPSVSAAPGQKVTISCSGSSSNIGNNFVSWYQQFPGTA |
| | | amino | PKLLIYDNNKRPSGIPDRFSGSKSGTSATLGITGLQTGDEADYY |
| | | acid | CGTWDSSLGAGVFGGGTKLTVL |
| | | sequence | |
| 1841 | YANG-2265 | LCDR1 | SSNIGNNF |
| 1842 | YANG-2265 | LCDR2 | DNN |
| 1843 | YANG-2265 | LCDR3 | GTWDSSLGAGV |
| 1844 | YANG-2266 | VH domain | CAGATTCAGTTGGTGCAGTCTGGGGCTGAGGTGAAGAAGCCTGG |
| | | nucleic | GGCCTCAGTGAAGCTTTCCTGCAAGACATCTGGATACACCTTCA |
| | | acid | CCAACTACTATATGGTCTGGGTGCGACAGGCCCCTGGACAAGGA |
| | | sequence | CTTGAGTGGATGGGCATAATCAAACCTAGTGGTGGTAACACAAT |
| | | | CTACGCACAGAAGTTCCAGGGCAGAGTCACCATGACCAGGGACA |
| | | | CGTCCACGAACACAGTCTACATGGACCTGAACAGTCTGAGATCT |
| | | | GAGGACACGGCCGTCTATTATTGTGCGAGAACTGGAGATAGGGC |
| | | | TTTTGATGTCTGGGGCCACGGGACAATGGTCACCGTGTCTTCA |
| 1845 | YANG-2266 | VH domain | QIQLVQSGAEVKKPGASVKLSCKTSGYTFTNYYMVWVRQAPGQG |
| | | amino | LEWMGIIKPSGGNTIYAQKFQGRVTMTRDTSTNTVYMDLNSLRS |
| | | acid | EDTAVYYCARTGDRAFDVWGHGTMVTVSS |
| | | sequence | |
| 1846 | YANG-2266 | HCDR1 | GYTFTNYY |
| 1847 | YANG-2266 | HCDR2 | IKPSGGNT |
| 1848 | YANG-2266 | HCDR3 | ARTGDRAFDV |
| 1849 | YANG-2266 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAATATTG |
| | | acid | GGAATAATTATGTATCCTGGTACCAACAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAACTGACCGTCCTA |
| 1850 | YANG-2266 | VL domain | QSVLTQPPSVSAAPGQKVTISCSGSSSNIGNNYVSWYQQFPGTA |
| | | amino | PKLLIYDNNKRPSGIPDRFSGSKSGTSATLGITGLQTGDEADYY |
| | | acid | CGTWDSSLGAGVFGGGTKLTVL |
| | | sequence | |
| 1851 | YANG-2266 | LCDR1 | SSNIGNNY |
| 1852 | YANG-2266 | LCDR2 | DNN |
| 1853 | YANG-2266 | LCDR3 | GTWDSSLGAGV |
| 1854 | YANG-2267 | VH domain | CAGATGCAATTGGTGCAGTCTGGGGCTGAGGTGAAGAAGCCTGG |
| | | nucleic | GGCCTCAGTGAAGATTTTCTGCAAGACATCTGGATACCCCTTCA |
| | | acid | GCAACTACTATATGGTCTGGGTGCGACAGGCCCCTGGACAAGGA |
| | | sequence | CTTGAGTGGCTGGGCATAATCAAACCTAGTGGTGGTAACACGAT |
| | | | CTACGCACAGAAGTTCCAGGGCAGAGTCACCATGACCAGGGACA |
| | | | CGTCCACGAATACAGTCTATTTGGACCTGACCAGTCTGAGATCT |
| | | | GAGGACACGGCCGTGTATTATTGTGCGCGAACTGGGGATAGGGC |
| | | | TTTTGATGTCTGGGGCCACGGGACAATGGTCACCGTCTCTTCA |
| 1855 | YANG-2267 | VH domain | QMQLVQSGAEVKKPGASVKIFCKTSGYPFSNYYMVWVRQAPGQG |
| | | amino | LEWLGIIKPSGGNTIYAQKFQGRVTMTRDTSTNTVYLDLTSLRS |
| | | acid | EDTAVYYCARTGDRAFDVWGHGTMVTVSS |
| | | sequence | |
| 1856 | YANG-2267 | HCDR1 | GYPFSNYY |
| 1857 | YANG-2267 | HCDR2 | IKPSGGNT |
| 1858 | YANG-2267 | HCDR3 | ARTGDRAFDV |
| 1859 | YANG-2267 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGTTCCAACATTG |
| | | acid | GGAGTAATTATGTTTCCTGGTACCAGCAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGCTGACCGTCCTA |
| 1860 | YANG-2267 | VL domain | QSVLTQPPSVSAAPGQKVTISCSGSSSNIGSNYVSWYQQFPGTA |
| | | amino | PKLLIYDNNKRPSGIPDRFSGSKSGTSATLGITGLQTGDEADYY |
| | | acid | CGTWDSSLGAGVFGGGTKLTVL |
| | | sequence | |
| 1861 | YANG-2267 | LCDR1 | SSNIGSNY |
| 1862 | YANG-2267 | LCDR2 | DNN |
| 1863 | YANG-2267 | LCDR3 | GTWDSSLGAGV |
| 1864 | YANG-2268 | VH domain | CAGATACAACTGGTGCAGTCTGGGGCTGAGGTGAAGAAGCCTGG |
| | | nucleic | GGCCTCAGTGAGGTTTTCCTGCAAGACATCTGGATACACCTTCA |
| | | acid | CCAACTACTATATGGTCTGGGTGCGACAGGCCCCTGGACAAGGA |
| | | sequence | CTTGAGTGGATGGGCATAATCAAACCTAGTGGTGGTAACACCAT |
| | | | CTACGCACAGAACTTCCAGGGCAGAGTCACCATGACCAGGGACA |
| | | | CGTCCACGAGCACAGTCTACATGGACCTGAGGAGTCTGAGATCT |
| | | | GAGGACACGGCCGTGTATTATTGTGCGAGAACTGGTGATAGGGC |
| | | | TTTTGATGTCTGGGGCCAAGGGACAATGGTCACCGTCTCTTCA |
| 1865 | YANG-2268 | VH domain | QIQLVQSGAEVKKPGASVRFSCKTSGYTFTNYYMVWVRQAPGQG |
| | | amino | LEWMGIIKPSGGNTIYAQNFQGRVTMTRDTSTSTVYMDLRSLRS |
| | | acid | EDTAVYYCARTGDRAFDVWGQGTMVTVSS |
| | | sequence | |
| 1866 | YANG-2268 | HCDR1 | GYTFTNYY |
| 1867 | YANG-2268 | HCDR2 | IKPSGGNT |
| 1868 | YANG-2268 | HCDR3 | ARTGDRAFDV |
| 1869 | YANG-2268 | VL domain | CAGTCTGTCTTGACTCAGCCGCCCTCAGTGTCTGCTGCCCCAGG |
| | | nucleic | ACAGAAGGTCAACATCTCCTGCTCTGGAAGCAGCTCCAACATTG |
| | | acid | GGAATAATTTTGTATCCTGGTACCAGCAGTTCTCAGGAACCGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGCTGACCGTCCTA |
| 1870 | YANG-2268 | VL domain | QSVLTQPPSVSAAPGQKVNISCSGSSSNIGNNFVSWYQQFSGTA |
| | | amino | PKLLIYDNNKRPSGIPDRFSGSKSGTSATLGITGLQTGDEADYY |
| | | acid | CGTWDSSLGAGVFGGGTKLTVL |
| | | sequence | |
| 1871 | YANG-2268 | LCDR1 | SSNIGNNE |
| 1872 | YANG-2268 | LCDR2 | DNN |
| 1873 | YANG-2268 | LCDR3 | GTWDSSLGAGV |
| 1874 | YANG-2269 | VH domain | CAGATGCAGTTGGTGCAGTCTGGGGCTGAGGTGAAGAAGCCTGG |
| | | nucleic | GGCCTCAGTGAAGATTTCCTGCAAGACATCTGGATATATCTTCA |
| | | acid | CCAAGTACTATATGGTCTGGGTGCGACAGGCCCCGGGACAAGGA |
| | | sequence | CTTGAGTGGATGGGCATAATCAAACCTAGTGGCGGTGACACGAT |
| | | | CTACGCACAGAAGTTCCAGGGCAGAGTCACCATGACCAGGGACA |
| | | | CGTCCACGAACACAGTCTATATGGACCTGAGCAGTCTGAGATCT |
| | | | GAGGACACGGCCGTGTATTATTGTGCGAGAACTGGAAATAGGGC |
| | | | TTTTGATGTCTGGGGCCACGGGACAATGGTCACCGTCTCTTCA |
| 1875 | YANG-2269 | VH domain | QMQLVQSGAEVKKPGASVKISCKTSGYIFTKYYMVWVRQAPGQG |
| | | amino | LEWMGIIKPSGGDTIYAQKFQGRVTMTRDTSTNTVYMDLSSLRS |
| | | acid | EDTAVYYCARTGNRAFDVWGHGTMVTVSS |
| | | sequence | |
| 1876 | YANG-2269 | HCDR1 | GYIFTKYY |
| 1877 | YANG-2269 | HCDR2 | IKPSGGDT |
| 1878 | YANG-2269 | HCDR3 | ARTGNRAFDV |
| 1879 | YANG-2269 | VL domain | CAGTCTGTGTTGACGCAGCCGCCCTCAGTGTCTGCGGCCCCAGG |
| | | nucleic | ACAGAAGGTCACCGTCTCCTGCTCTGGAAGCAGCTCCAACATTG |
| | | acid | GGAATAATTTTGTTTCCTGGTACCAGCAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGCTGGCCGTCCTT |
| 1880 | YANG-2269 | VL domain | QSVLTQPPSVSAAPGQKVTVSCSGSSSNIGNNFVSWYQQFPGTA |
| | | amino | PKLLIYDNNKRPSGIPDRFSGSKSGTSATLGITGLQTGDEADYY |
| | | acid | CGTWDSSLGAGVFGGGTKLAVL |
| | | sequence | |
| 1881 | YANG-2269 | LCDR1 | SSNIGNNF |
| 1882 | YANG-2269 | LCDR2 | DNN |
| 1883 | YANG-2269 | LCDR3 | GTWDSSLGAGV |
| 1884 | YANG-2270 | VH domain | CAGATTCAGTTGGTGCAGTCTGGGGCTGAGGTGAAGAAGCCTGG |
| | | nucleic | GGCCTCAGTGAGGTTTTCCTGCAAGACATCTGGATACACCTTCA |
| | | acid | CCAGCTACTATATGGTCTGGGTGCGACAGGCCCCTGGACAAGGA |
| | | sequence | CTTGAGTGGATGGGCATAATCAAACCTAGTGGTGGTAACACAAT |
| | | | CTACGCACAGAAGTTCCAGGGCAGAGTCACCATGACCAGGGACA |
| | | | CGTCCACGCACACAGTCTATATGGACCTGAGCAGTCTGAGATCT |
| | | | GAGGACACGGCCGTATATTATTGTGCGAGAACTGGTGATAGGGC |
| | | | TTTTGATGTCTGGGGCCACGGGACAATGATCACCGTCTCTTCA |
| 1885 | YANG-2270 | VH domain | QIQLVQSGAEVKKPGASVRFSCKTSGYTFTSYYMVWVRQAPGQG |
| | | amino | LEWMGIIKPSGGNTIYAQKFQGRVTMTRDTSTHTVYMDLSSLRS |
| | | acid | EDTAVYYCARTGDRAFDVWGHGTMITVSS |
| | | sequence | |
| 1886 | YANG-2270 | HCDR1 | GYTFTSYY |
| 1887 | YANG-2270 | HCDR2 | IKPSGGNT |
| 1888 | YANG-2270 | HCDR3 | ARTGDRAFDV |
| 1889 | YANG-2270 | VL domain | CAGTCTGTATTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GAAATAATTTTGTATCCTGGTACCAACAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGCTGACCGTCCTA |
| 1890 | YANG-2270 | VL domain | QSVLTQPPSVSAAPGQKVTISCSGSSSNIGNNFVSWYQQFPGTA |
| | | amino | PKLLIYDNNKRPSGIPDRFSGSKSGTSATLGITGLQTGDEADYY |
| | | acid | CGTWDSSLGAGVFGGGTKLTVL |
| | | sequence | |
| 1891 | YANG-2270 | LCDR1 | SSNIGNNF |
| 1892 | YANG-2270 | LCDR2 | DNN |
| 1893 | YANG-2270 | LCDR3 | GTWDSSLGAGV |
| 1894 | YANG-2271 | VH domain | CAGATACAGCTGGTGCAGTCTGGGGCTGAGGTGCAGAAGCCTGG |
| | | nucleic | GGCCTCAGTGAAGTTTTCCTGCAAGACATCTGGATACACCTTCA |
| | | acid | CCAACTTCTATATCGTCTGGGTGCGACAGGCCCCTGGACAAGGA |
| | | sequence | CTTGAGTGGATGGGCATAATCAAACCTAGTGGTGGTAACACAAT |
| | | | CTACGCACAGAATTTCCAGGGCAGAGTCACCATGACCAGGGACA |
| | | | CGTCCACGAACACAATCTACATGGACCTGAGGAGTCTGAGATCT |
| | | | GAGGACACGGCCGTGTATTATTGTGCGAGAACTGGGGATAGGGC |
| | | | TTTTGATGTCTGGGGCCAAGGGACAATGGTCACCGTCTCTTCA |
| 1895 | YANG-2271 | VH domain | QIQLVQSGAEVQKPGASVKFSCKTSGYTFTNFYIVWVRQAPGQG |
| | | amino | LEWMGIIKPSGGNTIYAQNFQGRVTMTRDTSINTIYMDLRSLRS |
| | | acid | EDTAVYYCARTGDRAFDVWGQGTMVTVSS |
| | | sequence | |
| 1896 | YANG-2271 | HCDR1 | GYTFTNFY |
| 1897 | YANG-2271 | HCDR2 | IKPSGGNT |
| 1898 | YANG-2271 | HCDR3 | ARTGDRAFDV |
| 1899 | YANG-2271 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTTTGTATCCTGGTACCAGCAGTTCCCAGGAACAGCC |
| | | sequence | CCCAAACTCCTCATTTATGACAATCATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGTTGACCGTCCTA |
| 1900 | YANG-2271 | VL domain | QSVLTQPPSVSAAPGQKVTISCSGSSSNIGNNFVSWYQQFPGTA |
| | | amino | PKLLIYDNHKRPSGIPDRFSGSKSGTSATLGITGLQTGDEADYY |
| | | acid | CGTWDSSLGAGVFGGGTKLTVL |
| | | sequence | |
| 1901 | YANG-2271 | LCDR1 | SSNIGNNF |
| 1902 | YANG-2271 | LCDR2 | DNH |
| 1903 | YANG-2271 | LCDR3 | GTWDSSLGAGV |
| 1904 | YANG-2272 | VH domain | CAGATGCAATTGGTGCAGTCTGGGGCTGAGGTGAAGAAGCCTGG |
| | | nucleic | GGCCTCAGTGAAGATTTCCTGCAAGACATCTGGATACACCTTCA |
| | | acid | CCAACTACTATATGGTCTGGGTGCGACAGGCCCCTGGACAAGGA |
| | | sequence | CTTGAGTGGATGGGCATAATCAAACCTAGTGGTGGTAACACGAT |
| | | | CTACGCACAGAAGTTCCAGGGCAGAGTCATCATGACCAGGGACA |
| | | | CGTCCACGAACACAGTCTATTTGGACCTGAACAGTCTGAGATCT |
| | | | GAGGACACGGCCGTGTATTATTGTGCGAAAACTGGGGATAGGGC |
| | | | TTTTAATGTCTGGGGCCACGGGACAATGGTCACCGTCTCTTCA |
| 1905 | YANG-2272 | VH domain | QMQLVQSGAEVKKPGASVKISCKTSGYTFTNYYMVWVRQAPGQG |
| | | amino | LEWMGIIKPSGGNTIYAQKFQGRVIMTRDTSTNTVYLDLNSLRS |
| | | acid | EDTAVYYCAKTGDRAFNVWGHGTMVTVSS |
| | | sequence | |
| 1906 | YANG-2272 | HCDR1 | GYTFTNYY |
| 1907 | YANG-2272 | HCDR2 | IKPSGGNT |
| 1908 | YANG-2272 | HCDR3 | AKTGDRAFNV |
| 1909 | YANG-2272 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTC |
| | | acid | GAAATAATTATGTTTCCTGGTACCAGCAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCTGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGCTGACCGTCCTT |
| 1910 | YANG-2272 | VL domain | QSVLTQPPSVSAAPGQKVTISCSGSSSNIRNNYVSWYQQFPGTA |
| | | amino | PKLLIYDNNKRPSGIPDRFSGSKSGTSATLGITGLQTGDEADYY |
| | | acid | CGTWDSSLGAGVFGGGTKLTVL |
| | | sequence | |
| 1911 | YANG-2272 | LCDR1 | SSNIRNNY |
| 1912 | YANG-2272 | LCDR2 | DNN |
| 1913 | YANG-2272 | LCDR3 | GTWDSSLGAGV |
| 1914 | YANG-2273 | VH domain | CAGATACAGCTGGTGCAGTCTGGGGCTGAGGTGAAGAAGCCTGG |
| | | nucleic | GGCCTCAGTGAAGTTTTCCTGCAAGACATCTGGATACACCTTCA |
| | | acid | CCAACTACTATATGGTCTGGGTGCGACAGGCCCCTGGACAAGGA |
| | | sequence | CTTGAGTGGATGGGCATAATCAAACCTAGTGGTGGTAACACAAT |
| | | | CTACGCACAGAATTTCCAGGGCAGAGTCACCATGACCAGGGACA |
| | | | CGTCCACGAACACAGTCTACATGGACCTGAGGAGTCTGAGATCT |
| | | | GAGGACACGGCCGTGTATTATTGTGCGAGAACTGGTGATAGGGC |
| | | | TTTTGATGTCTGGGGCCAAGGGACAATGGTCACCGTCTCTTCA |
| 1915 | YANG-2273 | VH domain | QIQLVQSGAEVKKPGASVKFSCKTSGYTFTNYYMVWVRQAPGQG |
| | | amino | LEWMGIIKPSGGNTIYAQNFQGRVTMTRDTSTNTVYMDLRSLRS |
| | | acid | EDTAVYYCARTGDRAFDVWGQGTMVTVSS |
| | | sequence | |
| 1916 | YANG-2273 | HCDR1 | GYTFTNYY |
| 1917 | YANG-2273 | HCDR2 | IKPSGGNT |
| 1918 | YANG-2273 | HCDR3 | ARTGDRAFDV |
| 1919 | YANG-2273 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTTTGTATCCTGGTACCAGCAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGCTGACCGTCCTA |
| 1920 | YANG-2273 | VL domain | QSVLTQPPSVSAAPGQKVTISCSGSSSNIGNNFVSWYQQFPGTA |
| | | amino | PKLLIYDNNKRPSGIPDRFSGSKSGTSATLGITGLQTGDEADYY |
| | | acid | CGTWDSSLGAGVFGGGTKLTVL |
| | | sequence | |
| 1921 | YANG-2273 | LCDR1 | SSNIGNNF |
| 1922 | YANG-2273 | LCDR2 | DNN |
| 1923 | YANG-2273 | LCDR3 | GTWDSSLGAGV |
| 1924 | YANG-2274 | VH domain | CAGCTGCAGCTGGTGCAGTCTGGGGCTGAGGTGAAGAAGCCTGG |
| | | nucleic | GGCCTCAGTGAAGATTTCCTGCAAGACATCTGGATACACCTTCA |
| | | acid | CCAGTTACTATATGGTCTGGGTGCGACAGGCCCCTGGACAAGGA |
| | | sequence | CTTGAATGGATGGGCATAATCAAACCTAGTGGTGGTAACACAAT |
| | | | CTACGCACAGAAGTTCCAGGGCAGAGTCACCATGACCAGGGACA |
| | | | CGTCCACGAATACAGTCTTCATGGACCTGAGGAGTCTGAGATCT |
| | | | GAAGACACGGCCGTGTATTATTGTGCGAGAACTGGGGATAGGGC |
| | | | TTTTGATGTCTGGGGCCAAGGGACACTGGTCACCGTCTCTACA |
| 1925 | YANG-2274 | VH domain | QLQLVQSGAEVKKPGASVKISCKTSGYTFTSYYMVWVRQAPGQG |
| | | amino | LEWMGIIKPSGGNTIYAQKFQGRVTMTRDTSTNTVFMDLRSLRS |
| | | acid | EDTAVYYCARTGDRAFDVWGQGTLVTVST |
| | | sequence | |
| 1926 | YANG-2274 | HCDR1 | GYTFTSYY |
| 1927 | YANG-2274 | HCDR2 | IKPSGGNT |
| 1928 | YANG-2274 | HCDR3 | ARTGDRAFDV |
| 1929 | YANG-2274 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTTTGTTTCCTGGTACCTGCAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCCAGCTGACCGTCCTA |
| 1930 | YANG-2274 | VL domain | QSVLTQPPSVSAAPGQKVTISCSGSSSNIGNNFVSWYLQFPGTA |
| | | amino | PKLLIYDNNKRPSGIPDRFSGSKSGTSATLGITGLQTGDEADYY |
| | | acid | CGTWDSSLGAGVFGGGTQLTVL |
| | | sequence | |
| 1931 | YANG-2274 | LCDR1 | SSNIGNNF |
| 1932 | YANG-2274 | LCDR2 | DNN |
| 1933 | YANG-2274 | LCDR3 | GTWDSSLGAGV |
| 1934 | YANG-2275 | VH domain | CAGATGCAGTTGGTGCAGTCTGGGGCTGAGGTGAAGAAGCCTGG |
| | | nucleic | GGCCTCAGTGAAGATTTCCTGCAAGACATCTGGATACACCTTCA |
| | | acid | CCAACTACTATATGGTCTGGGTGCGACAGGCCCCGGGACAAGGA |
| | | sequence | CTTGAGTGGATGGGCATAATCAAACCTAGTGGCGGTGACACGAT |
| | | | CTACGCACAGAAGTTCCAGGGCAGAGTCACCATGACCAGGGACA |
| | | | CGTCCACGAACACAGTCTATATGGACCTGACCAGTCTGAGATCT |
| | | | GAGGACACGGCCGTCTATTATTGTGCGAGAACTGGGAATAGGGC |
| | | | TTTTGATGTCTGGGGCCACGGGACAATGGTCACCGTCTCTTCA |
| 1935 | YANG-2275 | VH domain | QMQLVQSGAEVKKPGASVKISCKTSGYTFTNYYMVWVRQAPGQG |
| | | amino | LEWMGIIKPSGGDTIYAQKFQGRVTMTRDTSTNTVYMDLTSLRS |
| | | acid | EDTAVYYCARTGNRAFDVWGHGTMVTVSS |
| | | sequence | |
| 1936 | YANG-2275 | HCDR1 | GYTFTNYY |
| 1937 | YANG-2275 | HCDR2 | IKPSGGDT |
| 1938 | YANG-2275 | HCDR3 | ARTGNRAFDV |
| 1939 | YANG-2275 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAATATTG |
| | | acid | GGAATAATTATGTTTCCTGGTACCAGCAGTTCCCAGGAACAGCC |
| | | sequence | CCCAAACTCCTCATTTATGACAATAATAAGCGACCCTCAGGGAT |
| | | | TCCTGACCGATTCTCTGGCTCCAGGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGCTGACCGTCCTA |
| 1940 | YANG-2275 | VL domain | QSVLTQPPSVSAAPGQKVTISCSGSSSNIGNNYVSWYQQFPGTA |
| | | amino | PKLLIYDNNKRPSGIPDRESGSRSGTSATLGITGLQTGDEADYY |
| | | acid | CGTWDSSLGAGVFGGGTKLTVL |
| | | sequence | |
| 1941 | YANG-2275 | LCDR1 | SSNIGNNY |
| 1942 | YANG-2275 | LCDR2 | DNN |
| 1943 | YANG-2275 | LCDR3 | GTWDSSLGAGV |
| 1944 | YANG-2276 | VH domain | CAGATGCAATTGGTGCAGTCTGGGGCTGAGGTGAAGAAGCCTGG |
| | | nucleic | GGCCTCAGTGAAGATTTCCTGCAAGACATCTGGATACACCTTCA |
| | | acid | CCAACTACTATATGGTCTGGGTGCGACAGGCCCCTGGACAAGGA |
| | | sequence | CTTGAGTGGATGGGCATAATCAAACCTAGTGGTGGTAACACGAT |
| | | | CTACGCACAGAAGTTTCAGGGCAGAGTCACCATGGCCAGGGACA |
| | | | CGTCCACGAACACAGTCTATTTGGACCTGAGCAGTCTGAGATCT |
| | | | GAGGACACGGCCGTGTATTATTGTGCGAGAACTGGGGATAGGGC |
| | | | TTTTGATGTCTGGGGCCACGGGACAATGGTCACCGTCTCTTCA |
| 1945 | YANG-2276 | VH domain | QMQLVQSGAEVKKPGASVKISCKTSGYTFTNYYMVWVRQAPGQG |
| | | amino | LEWMGIIKPSGGNTIYAQKFQGRVTMARDTSTNTVYLDLSSLRS |
| | | acid | EDTAVYYCARTGDRAFDVWGHGTMVTVSS |
| | | sequence | |
| 1946 | YANG-2276 | HCDR1 | GYTFTNYY |
| 1947 | YANG-2276 | HCDR2 | IKPSGGNT |
| 1948 | YANG-2276 | HCDR3 | ARTGDRAFDV |
| 1949 | YANG-2276 | VL domain | CAGTCTGTGTTGACGCAGCCGCCCTCAGTGTCTGCGGCTCCAGG |
| | | nucleic | ACAGAAGGTCACCATCTCCTGCTCTGGAAGCAGCTCCAACATTG |
| | | acid | GGAATAATTATGTTTCCTGGTACCAGCAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGTCACCC |
| | | | TGGGCATCACCGGACTCCAGACTGGGGACGAGGCCGATTATTAC |
| | | | TGCGGAACATGGGATATCAGCCTGGGTGCTGGGGTGTTCGGCGG |
| | | | AGGGACCAAGCTGACCGTCCTA |
| 1950 | YANG-2276 | VL domain | QSVLTQPPSVSAAPGQKVTISCSGSSSNIGNNYVSWYQQFPGTA |
| | | amino | PKLLIYDNNKRPSGIPDRFSGSKSGTSVTLGITGLQTGDEADYY |
| | | acid | CGTWDISLGAGVFGGGTKLTVL |
| | | sequence | |
| 1951 | YANG-2276 | LCDR1 | SSNIGNNY |
| 1952 | YANG-2276 | LCDR2 | DNN |
| 1953 | YANG-2276 | LCDR3 | GTWDISLGAGV |
| 1954 | YANG-2277 | VH domain | CAGATACAGTTGGTGCAGTCTGGGGCTGAGGTGAAGAAGCCTGG |
| | | nucleic | GGCCTCAGTGAAGTTTTCCTGCAAGACATCTGGATACACCTTCA |
| | | acid | CCAACTACTATATGGTCTGGGTGCGACAGGCCCCTGGACAAGGA |
| | | sequence | CTTGAGTGGATGGGCATAATCAAACCTAGTGGTGGTAACACAAT |
| | | | CTACGCACAGAATTTCCAGGGCAGAGTCACCATGACCAGGGACA |
| | | | CGTCCACGAACACAGTCTACATGGACCTGAGGAGTCTGAGATCT |
| | | | GAGGACACGGCCGTGTATTATTGTGCGAGAACTGCTGATAGGGC |
| | | | TTTTGATGTCTGGGGCCAAGGGACAATGGTCACCGTCTCTTCA |
| 1955 | YANG-2277 | VH domain | QIQLVQSGAEVKKPGASVKFSCKTSGYTFTNYYMVWVRQAPGQG |
| | | amino | LEWMGIIKPSGGNTIYAQNFQGRVTMTRDTSTNTVYMDLRSLRS |
| | | acid | EDTAVYYCARTADRAFDVWGQGTMVTVSS |
| | | sequence | |
| 1956 | YANG-2277 | HCDR1 | GYTFTNYY |
| 1957 | YANG-2277 | HCDR2 | IKPSGGNT |
| 1958 | YANG-2277 | HCDR3 | ARTADRAFDV |
| 1959 | YANG-2277 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTTTGTATCCTGGTACCAGCAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCATGTTGACCGTCCTA |
| 1960 | YANG-2277 | VL domain | QSVLTQPPSVSAAPGQKVTISCSGSSSNIGNNFVSWYQQFPGTA |
| | | amino | PKLLIYDNNKRPSGIPDRFSGSKSGTSATLGITGLQTGDEADYY |
| | | acid | CGTWDSSLGAGVFGGGTMLTVL |
| | | sequence | |
| 1961 | YANG-2277 | LCDR1 | SSNIGNNF |
| 1962 | YANG-2277 | LCDR2 | DNN |
| 1963 | YANG-2277 | LCDR3 | GTWDSSLGAGV |
| 1964 | YANG-2278 | VH domain | CAGATTCAGTTGGTGCAGTCTGGGGCTGAGGTGAAGAAGCCTGG |
| | | nucleic | GGCCTCAGTGAAGCTTTCCTGCAAGACATCTGGATACACCTTCA |
| | | acid | CCAGCTACTATATGGTCTGGGTGCGACAGGCCCCTGGACAAGGA |
| | | sequence | CTTGAGTGGATGGGCATAATCAAACCTAGTGGTGGTAACACAAT |
| | | | CTACGCACAGAAGTTCCAGGGCAGAGTCACCATGACCAGGGACA |
| | | | CGTCCACGAACACAGTCTACATGGACCTGAACAGTCTGAGATCT |
| | | | GAGGACACGGCCGTCTATTATTGTGCGAGAACTGGAGATAGGGC |
| | | | TTTTGATGTCTGGGGCCACGGGACAATGGTCACCGTGTCTTCA |
| 1965 | YANG-2278 | VH domain | QIQLVQSGAEVKKPGASVKLSCKTSGYTFTSYYMVWVRQAPGQG |
| | | amino | LEWMGIIKPSGGNTIYAQKFQGRVTMTRDTSINTVYMDLNSLRS |
| | | acid | EDTAVYYCARTGDRAFDVWGHGTMVTVSS |
| | | sequence | |
| 1966 | YANG-2278 | HCDR1 | GYTFTSYY |
| 1967 | YANG-2278 | HCDR2 | IKPSGGNT |
| 1968 | YANG-2278 | HCDR3 | ARTGDRAFDV |
| 1969 | YANG-2278 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTATGTATCCTGGTACCAGCAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGCTGACCGTCCTA |
| 1970 | YANG-2278 | VL domain | QSVLTQPPSVSAAPGQKVTISCSGSSSNIGNNYVSWYQQFPGTA |
| | | amino | PKLLIYDNNKRPSGIPDRFSGSKSGTSATLGITGLQTGDEADYY |
| | | acid | CGTWDSSLGAGVFGGGTKLTVL |
| | | sequence | |
| 1971 | YANG-2278 | LCDR1 | SSNIGNNY |
| 1972 | YANG-2278 | LCDR2 | DNN |
| 1973 | YANG-2278 | LCDR3 | GTWDSSLGAGV |
| 1974 | YANG-2279 | VH domain | CAGATGCAATTGGTGCAGTCTGGGGCTGAGGTGAAGAAGCCTGG |
| | | nucleic | GGCCTCAGTGAAGATTTCCTGCAAGACATCTGGATACACCTTCA |
| | | acid | CCAACTACTATATGGTCTGGGTGCGACAGGCCCCTGGACAAGGA |
| | | sequence | CTTGAGTGGATGGGCATAATCAAACCTAGTGGTGGTAACGCGAT |
| | | | CTACGCACAGAAGTTCCAGGGCAGAGTCACCATGACCAGGGACA |
| | | | CGTCCACGAACACAGTCTATTTGGACCTGAGCAGTCTGAGATCT |
| | | | GAGGACACGGCCGTGTACTATTGTGCGAGAACTGGAGATAGGGC |
| | | | TTTTGATGTCTGGGGCCACGGGACAATGGTCACCGTCTCTTCA |
| 1975 | YANG-2279 | VH domain | QMQLVQSGAEVKKPGASVKISCKTSGYTFTNYYMVWVRQAPGQG |
| | | amino | LEWMGIIKPSGGNAIYAQKFQGRVTMTRDTSTNTVYLDLSSLRS |
| | | acid | EDTAVYYCARTGDRAFDVWGHGTMVTVSS |
| | | sequence | |
| 1976 | YANG-2279 | HCDR1 | GYTFTNYY |
| 1977 | YANG-2279 | HCDR2 | IKPSGGNA |
| 1978 | YANG-2279 | HCDR3 | ARTGDRAFDV |
| 1979 | YANG-2279 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTATGTTTCCTGGTACCAGCAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGCTGACCGTCCTA |
| 1980 | YANG-2279 | VL domain | QSVLTQPPSVSAAPGQKVTISCSGSSSNIGNNYVSWYQQFPGTA |
| | | amino | PKLLIYDNNKRPSGIPDRFSGSKSGTSATLGITGLQTGDEADYY |
| | | acid | CGTWDSSLGAGVFGGGTKLTVL |
| | | sequence | |
| 1981 | YANG-2279 | LCDR1 | SSNIGNNY |
| 1982 | YANG-2279 | LCDR2 | DNN |
| 1983 | YANG-2279 | LCDR3 | GTWDSSLGAGV |
| 1984 | YANG-2280 | VH domain | CAGATACAGCTGGTGCAGTCTGGGGCTGAGGTGAAGAAGCCTGG |
| | | nucleic | GGCCTCAGTGAAGTTTTCCTGCAAGACATCTGGATACATTTTCA |
| | | acid | CCAACTACTATATGGTCTGGGTGCGACAGGCCCCTGGACAAGGA |
| | | sequence | CTTGAGTGGATGGGCATAATCAAACCTAGTGGAGGTAACACAAT |
| | | | CTACGCACAGAAGTTCCAGGGCAGAGTCACCATGACCAGGGACA |
| | | | CGTCCACTAACACAGTCTACATGGACCTGAATAATCTGAGATCT |
| | | | GAGGACACGGCCGTGTATTTTTGTGCGAGAACTGGTGATAGGGC |
| | | | TTTTGATGTCTGGGGCCAAGGGTCAATGGTCACCGTCTCTTCA |
| 1985 | YANG-2280 | VH domain | QIQLVQSGAEVKKPGASVKFSCKTSGYIFTNYYMVWVRQAPGQG |
| | | amino | LEWMGIIKPSGGNTIYAQKFQGRVTMTRDTSTNTVYMDLNNLRS |
| | | acid | EDTAVYFCARTGDRAFDVWGQGSMVTVSS |
| | | sequence | |
| 1986 | YANG-2280 | HCDR1 | GYIFTNYY |
| 1987 | YANG-2280 | HCDR2 | IKPSGGNT |
| 1988 | YANG-2280 | HCDR3 | ARTGDRAFDV |
| 1989 | YANG-2280 | VL domain | CAGTCTGTGTTGACGCAGCCGCCCTCAATGTCTGCGGCCCCAGG |
| | | nucleic | ACAGAAGGTCACCATCTCCTGCTCTGGAAGCAGCTCCAACATTG |
| | | acid | GGAATAATTTTGTATCCTGGTACCGGCAGTTCCCCGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGGACATGGGATAGCAGCCTGAGTGCTGGGGTGTTCGGCGG |
| | | | AGGGACCAAGCTGACCGTCCTA |
| 1990 | YANG-2280 | VL domain | QSVLTQPPSMSAAPGQKVTISCSGSSSNIGNNFVSWYRQFPGTA |
| | | amino | PKLLIYDNNKRPSGIPDRFSGSKSGTSATLGITGLQTGDEADYY |
| | | acid | CGTWDSSLSAGVFGGGTKLTVL |
| | | sequence | |
| 1991 | YANG-2280 | LCDR1 | SSNIGNNF |
| 1992 | YANG-2280 | LCDR2 | DNN |
| 1993 | YANG-2280 | LCDR3 | GTWDSSLSAGV |
| 1994 | YANG-2281 | VH domain | CAGATGCAGTTGGTGCAGTCTGGGGCTGAGGTGAAGAAGCCTGG |
| | | nucleic | GGCCTCAGTGAAGATTTCCTGCAAGACATCTGGATATATTTTCA |
| | | acid | CCAACTACTATATGGTCTGGGTGCGACAGGCCTCTGGACAAGGA |
| | | sequence | CTTGAGTGGATGGGCATAATCAAACCTAGTGGTGGTAACACGAT |
| | | | CTACGCACAGAGGTTCCAGGGCAGAGTCACCATGACCAGAGACA |
| | | | CGTCCACGAACACAGTCTATATGGACCTGAGCAGTCTGAGATCT |
| | | | GAGGACACGGCCGTGTATTATTGTGCGAGAACTGGGAATAGGGC |
| | | | TTTTGATGTCTGGGGCCACGGGACAATGGTCACCGTCTCTTCA |
| 1995 | YANG-2281 | VH domain | QMQLVQSGAEVKKPGASVKISCKTSGYIFTNYYMVWVRQASGQG |
| | | amino | LEWMGIIKPSGGNTIYAQRFQGRVTMTRDTSTNTVYMDLSSLRS |
| | | acid | EDTAVYYCARTGNRAFDVWGHGTMVTVSS |
| | | sequence | |
| 1996 | YANG-2281 | HCDR1 | GYIFTNYY |
| 1997 | YANG-2281 | HCDR2 | IKPSGGNT |
| 1998 | YANG-2281 | HCDR3 | ARTGNRAFDV |
| 1999 | YANG-2281 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | CGAATAATTATGTTTCCTGGTACCAGCAGTTCCCAGGAACAGCC |
| | | sequence | CCCAAACTCCTCATTTTTGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGCTGACCGTCCTA |
| 2000 | YANG-2281 | VL domain | QSVLTQPPSVSAAPGQKVTISCSGSSSNIANNYVSWYQQFPGTA |
| | | amino | PKLLIFDNNKRPSGIPDRFSGSKSGTSATLGITGLQTGDEADYY |
| | | acid | CGTWDSSLGAGVFGGGTKLTVL |
| | | sequence | |
| 2001 | YANG-2281 | LCDR1 | SSNIANNY |
| 2002 | YANG-2281 | LCDR2 | DNN |
| 2003 | YANG-2281 | LCDR3 | GTWDSSLGAGV |
| 2004 | YANG-2282 | VH domain | CAGATTCAGTTGGTGCAGTCTGGGGCTGAGGTGAAGAAGCCTGG |
| | | nucleic | GGCCTCAGTGAAGTTTTCCTGCAAGACATCTGGATACACCTTCA |
| | | acid | CCAGTTACTATATGGTCTGGGTGCGACAGGCCCCTGGACAAGGA |
| | | sequence | CTTGAGTGGATGGGCATAATCAAACCTAGTGGTGGTAACACAAT |
| | | | CTACGCACAGAAGTTCCAGGGCAGAGTCACCATGACCAGGGACA |
| | | | CGTCCACGAACACAGTCTACATGGACCTGAACAGTCTGAGATCT |
| | | | GAGGACACGGCCGTCTATTATTGTGCGAGAACTGGAGATAGGGC |
| | | | TTTTGATGTCTGGGGCCACGGGACAATGGTCACCGTGTCTTCA |
| 2005 | YANG-2282 | VH domain | QIQLVQSGAEVKKPGASVKFSCKTSGYTFTSYYMVWVRQAPGQG |
| | | amino | LEWMGIIKPSGGNTIYAQKFQGRVTMTRDTSTNTVYMDLNSLRS |
| | | acid | EDTAVYYCARTGDRAFDVWGHGTMVTVSS |
| | | sequence | |
| 2006 | YANG-2282 | HCDR1 | GYTFTSYY |
| 2007 | YANG-2282 | HCDR2 | IKPSGGNT |
| 2008 | YANG-2282 | HCDR3 | ARTGDRAFDV |
| 2009 | YANG-2282 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTATGTATCCTGGTACCAGCAGTTTGCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCGCCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAACTGACCGTCCTA |
| 2010 | YANG-2282 | VL domain | QSVLTQPPSVSAAPGQKVTISCSGSSSNIGNNYVSWYQQFAGTA |
| | | amino | PKLLIYDNNKRPSGIPDRFSGSKSGTSATLGIAGLQTGDEADYY |
| | | acid | CGTWDSSLGAGVFGGGTKLTVL |
| | | sequence | |
| 2011 | YANG-2282 | LCDR1 | SSNIGNNY |
| 2012 | YANG-2282 | LCDR2 | DNN |
| 2013 | YANG-2282 | LCDR3 | GTWDSSLGAGV |
| 2014 | YANG-2283 | VH domain | CAGATTCAGTTGGTGCAGTCTGGGGCTGAGGTGAAGAAGCCTGG |
| | | nucleic | GGCCTCAGTGAAGTTTTCCTGCAAGACATCTGGATACAGCTTCA |
| | | acid | TCAATTATTATATGGTCTGGGTGCGCCAGGCCCCTGGACAAGGA |
| | | sequence | CTTGAGTGGATGGGCATAATCAAACCTAGTGGTGGTAACACAAT |
| | | | CTACGCACAGAAGTTCCAGGGCAGAGTCACCATGACCAGGGACA |
| | | | CGTCCACGAACACAGTCTACATGGACCTGCGCAGTCTGAGATCT |
| | | | GAGGACACGGCCGTATATTATTGTGCGAGAACTGGGGATAGGGC |
| | | | TTTTGATGTCTGGGGCCACGGGACAATGATCACCGTCTCTTCA |
| 2015 | YANG-2283 | VH domain | QIQLVQSGAEVKKPGASVKFSCKTSGYSFINYYMVWVRQAPGQG |
| | | amino | LEWMGIIKPSGGNTIYAQKFQGRVTMTRDTSTNTVYMDLRSLRS |
| | | acid | EDTAVYYCARTGDRAFDVWGHGTMITVSS |
| | | sequence | |
| 2016 | YANG-2283 | HCDR1 | GYSFINYY |
| 2017 | YANG-2283 | HCDR2 | IKPSGGNT |
| 2018 | YANG-2283 | HCDR3 | ARTGDRAFDV |
| 2019 | YANG-2283 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAACTCCAACATTG |
| | | acid | GGAATAATTTTGTATCCTGGTACCAACAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGTTGACCGTCCTA |
| 2020 | YANG-2283 | VL domain | QSVLTQPPSVSAAPGQKVTISCSGSNSNIGNNFVSWYQQFPGTA |
| | | amino | PKLLIYDNNKRPSGIPDRFSGSKSGTSATLGITGLQTGDEADYY |
| | | acid | CGTWDSSLGAGVFGGGTKLTVL |
| | | sequence | |
| 2021 | YANG-2283 | LCDR1 | NSNIGNNE |
| 2022 | YANG-2283 | LCDR2 | DNN |
| 2023 | YANG-2283 | LCDR3 | GTWDSSLGAGV |
| 2024 | YANG-2284 | VH domain | CAGATTCAGTTGGTGCAGTCTGGGGCTGAGGTGAAGAAGCCTGG |
| | | nucleic | GGCCTCAGTGAAGTTTTCCTGCAAGACATCTGGATACACCTTCA |
| | | acid | CCAGCTACTATATGGTCTGGGTGCGACAGGCCCCTGGACAAGGA |
| | | sequence | CTTGAGTGGATGGGCATAATCAAACCTAGTGGTGGTAACACAAT |
| | | | CTACGCACAGAAGTTCCAGGGCAGAGTCACCATGACCAGGGACA |
| | | | CGTCCACGAACACAGTCTACATGGACCTGAACAGTCTGAGATCT |
| | | | GAGGACACGGCCGTCTATTATTGTGCGAGAACTGGAGATAGGGC |
| | | | TTTTGATGTCTGGGGCCACGGGACAATGGTCACCGTGTCTTCA |
| 2025 | YANG-2284 | VH domain | QIQLVQSGAEVKKPGASVKFSCKTSGYTFTSYYMVWVRQAPGQG |
| | | amino | LEWMGIIKPSGGNTIYAQKFQGRVTMTRDTSTNTVYMDLNSLRS |
| | | acid | EDTAVYYCARTGDRAFDVWGHGTMVTVSS |
| | | sequence | |
| 2026 | YANG-2284 | HCDR1 | GYTFTSYY |
| 2027 | YANG-2284 | HCDR2 | IKPSGGNT |
| 2028 | YANG-2284 | HCDR3 | ARTGDRAFDV |
| 2029 | YANG-2284 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTATGTATCCTGGTACCAGCAGTTCCCAGGAACAGCC |
| | | sequence | CCCAAACTCCTCATTTATGACAATAATAAGCGACCCTCAGGGAT |
| | | | TCCTGACCGATTCTCTGCTTCCAAGACTGGCACGTCAGCCACCC |
| | | | TGGGCATCACCGGACCCCAGACTGGGGACGAGGCCGATTATTAC |
| | | | TGCGGAACATGGGATAGCAGCCTGGGTGCTGGGGTGTTCGGCGG |
| | | | AGGGACCAAACTGACCGTCCTA |
| 2030 | YANG-2284 | VL domain | QSVLTQPPSVSAAPGQKVTISCSGSSSNIGNNYVSWYQQFPGTA |
| | | amino | PKLLIYDNNKRPSGIPDRFSASKTGTSATLGITGPQTGDEADYY |
| | | acid | CGTWDSSLGAGVFGGGTKLTVL |
| | | sequence | |
| 2031 | YANG-2284 | LCDR1 | SSNIGNNY |
| 2032 | YANG-2284 | LCDR2 | DNN |
| 2033 | YANG-2284 | LCDR3 | GTWDSSLGAGV |
| 2034 | YANG-2285 | VH domain | CAAATGCAGTTGGTGCAGTCTGGGGCTGAGGTGAAGAAGCCTGG |
| | | nucleic | GGCCTCAGTGAAGATTTCCTGCAAGACATCTGGATACACCTTCA |
| | | acid | TCAACTACTATATGGTCTGGGTGCGACAGGCCCCTGGACAAGGA |
| | | sequence | CTTGAGTGGATGGGCATAATCAATCCTAGTGGTGGTAATACGGT |
| | | | CTACGCACAGAAGTTCCAGGGCAGAGTCACCATGACCAGGGACA |
| | | | CGTCCACGAACACAGTCTATATGGACCTGAGCAGTCTGAGATCT |
| | | | GAGGACACGGCCGTCTATTATTGTGCGAGAACTGGTGATAGGGC |
| | | | TTTTGATGTCTGGGGCCACGGGACAATGGTCACCGTCTCTTCA |
| 2035 | YANG-2285 | VH domain | QMQLVQSGAEVKKPGASVKISCKTSGYTFINYYMVWVRQAPGQG |
| | | amino | LEWMGIINPSGGNTVYAQKFQGRVTMTRDTSTNTVYMDLSSLRS |
| | | acid | EDTAVYYCARTGDRAFDVWGHGTMVTVSS |
| | | sequence | |
| 2036 | YANG-2285 | HCDR1 | GYTFINYY |
| 2037 | YANG-2285 | HCDR2 | INPSGGNT |
| 2038 | YANG-2285 | HCDR3 | ARTGDRAFDV |
| 2039 | YANG-2285 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTATGTTTCCTGGTACCAGCAGTTCCCAGGAACAGCC |
| | | sequence | CCCAAACTCCTCATTTATGACAATCATAGGCGACCCTCAGGGAT |
| | | | TCCTGACCGATTCTCTGCCTCCAAGTCTGGCACGTCAGCCACCC |
| | | | TGGGCATCACCGGACTCCTGACTGGGGACGAGGCCGATTATTAC |
| | | | TGCGGAACATGGGATAGCGGCCTGGGTGCTGGGGTGTTCGGCGG |
| | | | AGGGACCAAGCTGACCGTCCTA |
| 2040 | YANG-2285 | VL domain | QSVLTQPPSVSAAPGQKVTISCSGSSSNIGNNYVSWYQQFPGTA |
| | | amino | PKLLIYDNHRRPSGIPDRESASKSGTSATLGITGLLTGDEADYY |
| | | acid | CGTWDSGLGAGVFGGGTKLTVL |
| | | sequence | |
| 2041 | YANG-2285 | LCDR1 | SSNIGNNY |
| 2042 | YANG-2285 | LCDR2 | DNH |
| 2043 | YANG-2285 | LCDR3 | GTWDSGLGAGV |
| 2044 | YANG-2286 | VH domain | CAGATGCAATTGGTGCAGTCTGGGGCTGAGGTGAAGAAGCCTGG |
| | | nucleic | GGCCTCAGTGAAGATTTCCTGCAAGACATCTGGATACACCTTCA |
| | | acid | CCAATTACTATATGGTCTGGGTGCGACAGGCCCCTGGACAAGGA |
| | | sequence | CTTGAGTGGATGGGCATAATCAAACCTAGTGGTGGTAACACGAT |
| | | | CTACGCACAGAAGTTCCAGGGCAGAGTCACCATGACCAGGGACA |
| | | | CGTCCACGACCACAGTCTATTTGGACCTGAACAGTCTGAGATCT |
| | | | GAGGACACGGCCGTGTATTATTGTGCGAGAACTGGGGATAGGGC |
| | | | TTTTGATGTCTGGGGCCACGGGACAATGGTCACCGTCTCTTCA |
| 2045 | YANG-2286 | VH domain | QMQLVQSGAEVKKPGASVKISCKTSGYTFTNYYMVWVRQAPGQG |
| | | amino | LEWMGIIKPSGGNTIYAQKFQGRVTMTRDTSTTTVYLDLNSLRS |
| | | acid | EDTAVYYCARTGDRAFDVWGHGTMVTVSS |
| | | sequence | |
| 2046 | YANG-2286 | HCDR1 | GYTFTNYY |
| 2047 | YANG-2286 | HCDR2 | IKPSGGNT |
| 2048 | YANG-2286 | HCDR3 | ARTGDRAFDV |
| 2049 | YANG-2286 | VL domain | CAGTCTGTGTTGACGCAGCCGCCCTCAGTGTCTGCGGCCCCAGG |
| | | nucleic | ACAGAAGGTCACCATCTCCTGCTCTGGAACCAGCTCCAACATTG |
| | | acid | GGAATAATTATGTTTCCTGGTACCAGCAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCACGCTGACCGTCCTA |
| 2050 | YANG-2286 | VL domain | QSVLTQPPSVSAAPGQKVTISCSGTSSNIGNNYVSWYQQFPGTA |
| | | amino | PKLLIYDNNKRPSGIPDRFSGSKSGTSATLGITGLQTGDEADYY |
| | | acid | CGTWDSSLGAGVFGGGTTLTVL |
| | | sequence | |
| 2051 | YANG-2286 | LCDR1 | SSNIGNNY |
| 2052 | YANG-2286 | LCDR2 | DNN |
| 2053 | YANG-2286 | LCDR3 | GTWDSSLGAGV |
| 2054 | YANG-2287 | VH domain | CAGATACAACTGGTGCAGTCTGGGGCTGAGGTGAAGAAGCCTGG |
| | | nucleic | GGCCTCAGTGAAGTTTTCCTGCAAGACATCTGGATACACCTTCA |
| | | acid | CCAACTACTATATGGTCTGGGTGCGACAGGCCCCTGGACAAGGA |
| | | sequence | CTTGAGTGGATGGGCATAATCAAACCTAGTGGTGGTAACACAAT |
| | | | CTACGCACAGAACTTCCAGGGCAGAGTCACCATGACCAGGGACA |
| | | | CGTCCACGAACACAGTCTACATGGACCTGAGGACTCTGAGATCT |
| | | | GAGGACACGGCCGTTTATTATTGTGCGAGAACTGGTGATAGGGC |
| | | | TTTTGATGTCTGGGGCCAAGGGACAATGGTCACCGTCTCTTCA |
| 2055 | YANG-2287 | VH domain | QIQLVQSGAEVKKPGASVKFSCKTSGYTFTNYYMVWVRQAPGQG |
| | | amino | LEWMGIIKPSGGNTIYAQNFQGRVTMTRDTSTNTVYMDLRTLRS |
| | | acid | EDTAVYYCARTGDRAFDVWGQGTMVTVSS |
| | | sequence | |
| 2056 | YANG-2287 | HCDR1 | GYTFTNYY |
| 2057 | YANG-2287 | HCDR2 | IKPSGGNT |
| 2058 | YANG-2287 | HCDR3 | ARTGDRAFDV |
| 2059 | YANG-2287 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTTTGTTTCCTGGTACCAGCAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGATTCCAGACTGGGGACGAGGCCGATTATTAC |
| | | | TGCGGAACATGGGATAGCAGCCTGGGTGCTGGGGTGTTCGGCGG |
| | | | AGGGACCAGGCTGACCGTCCTA |
| 2060 | YANG-2287 | VL domain | QSVLTQPPSVSAAPGQKVTISCSGSSSNIGNNFVSWYQQFPGTA |
| | | amino | PKLLIYDNNKRPSGIPDRFSGSKSGTSATLGITGFQTGDEADYY |
| | | acid | CGTWDSSLGAGVFGGGTRLTVL |
| | | sequence | |
| 2061 | YANG-2287 | LCDR1 | SSNIGNNF |
| 2062 | YANG-2287 | LCDR2 | DNN |
| 2063 | YANG-2287 | LCDR3 | GTWDSSLGAGV |
| 2064 | YANG-2288 | VH domain | CAGATGCAGCTGGTACAGTCTGGGGCTGAGGTGAAGAAGCCTGG |
| | | nucleic | GGCCTCAGTGAAGATTTCCTGCAAGACATCTGGATACACCTTCA |
| | | acid | CCAGCTACTATATGGTCTGGGTGCGACAGGCCCCTGGACAAGGA |
| | | sequence | CTTGAGTGGATGGGCATAATCAAACCTAGTGGTGGTAACACAAT |
| | | | CTATGCACAGAAGTTCCAGGGCAGAGTCAACATGACCAGGGACA |
| | | | CGTCCACGAACACAGTCTATATGGACCTGAGTAGTCTGAGATCT |
| | | | GAGGACACGGCCGTGTATTATTGTGCGAGAACTGGGGATAGGGC |
| | | | TTTTGATGTCTGGGGCCAAGGGACAATGGTCACCGTCTCTTCA |
| 2065 | YANG-2288 | VH domain | QMQLVQSGAEVKKPGASVKISCKTSGYTFTSYYMVWVRQAPGQG |
| | | amino | LEWMGIIKPSGGNTIYAQKFQGRVNMTRDTSTNTVYMDLSSLRS |
| | | acid | EDTAVYYCARTGDRAFDVWGQGTMVTVSS |
| | | sequence | |
| 2066 | YANG-2288 | HCDR1 | GYTFTSYY |
| 2067 | YANG-2288 | HCDR2 | IKPSGGNT |
| 2068 | YANG-2288 | HCDR3 | ARTGDRAFDV |
| 2069 | YANG-2288 | VL domain | CAGTCTGTGTTGACGCAGCCGCCCTCAGTGTCTGCGGCCCCAGG |
| | | nucleic | ACAGAAGGTCACCATCTCCTGCTCTGGAAGTACCTCCAACATTG |
| | | acid | GGACTAATTATGTCTCCTGGTACCAGCAGCTCCCAGGAACAGCC |
| | | sequence | CCCAAACTCCTCATTTATGACAATAGTAAGCGCCCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAACTGACCGTCCTA |
| 2070 | YANG-2288 | VL domain | QSVLTQPPSVSAAPGQKVTISCSGSTSNIGTNYVSWYQQLPGTA |
| | | amino | PKLLIYDNSKRPSGIPDRFSGSKSGTSATLGITGLQTGDEADYY |
| | | acid | CGTWDSSLGAGVFGGGTKLTVL |
| | | sequence | |
| 2071 | YANG-2288 | LCDR1 | TSNIGTNY |
| 2072 | YANG-2288 | LCDR2 | DNS |
| 2073 | YANG-2288 | LCDR3 | GTWDSSLGAGV |
| 2074 | YANG-2289 | VH domain | CAGATGCAATTGGTGCAGTCTGGGGCTGAGGTGAAGAAGCCTGG |
| | | nucleic | GGCCTCAGTGAAGATTTCCTGCAAGACATCTGGATACACCTTCA |
| | | acid | CCAACTACTATATGGTCTGGGTGCGACAGGCCCCTGGACAAGGA |
| | | sequence | CTTGAATGGATGGGCATAATCAAACCTAGTGGTGGTAACACGAT |
| | | | CTACGCACAGAAGTTCCAGGGCAGAGTCACCATGACCAGGGACA |
| | | | CGTCCACGAACACAGTCTATTTGGACCTGACCAGTCTGAGATCT |
| | | | GAGGACACGGCCGTGTATTATTGTGCGAGAACTGGGGATAGGGC |
| | | | TTTTGATGTCTGGGGCCACGGGACAATGGTCACCGTCTCTTCA |
| 2075 | YANG-2289 | VH domain | QMQLVQSGAEVKKPGASVKISCKTSGYTFTNYYMVWVRQAPGQG |
| | | amino | LEWMGIIKPSGGNTIYAQKFQGRVTMTRDTSTNTVYLDLTSLRS |
| | | acid | EDTAVYYCARTGDRAFDVWGHGTMVTVSS |
| | | sequence | |
| 2076 | YANG-2289 | HCDR1 | GYTFTNYY |
| 2077 | YANG-2289 | HCDR2 | IKPSGGNT |
| 2078 | YANG-2289 | HCDR3 | ARTGDRAFDV |
| 2079 | YANG-2289 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GAAATAATTATGTTTCCTGGTACCAGCAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGCTGACCGTCCTA |
| 2080 | YANG-2289 | VL domain | QSVLTQPPSVSAAPGQKVTISCSGSSSNIGNNYVSWYQQFPGTA |
| | | amino | PKLLIYDNNKRPSGIPDRFSGSKSGTSATLGITGLQTGDEADYY |
| | | acid | CGTWDSSLGAGVFGGGTKLTVL |
| | | sequence | |
| 2081 | YANG-2289 | LCDR1 | SSNIGNNY |
| 2082 | YANG-2289 | LCDR2 | DNN |
| 2083 | YANG-2289 | LCDR3 | GTWDSSLGAGV |
| 2084 | YANG-2290 | VH domain | CAGATGCAATTGGTGCAGTCTGGGACTGAGGTGAAGAAGCCTGG |
| | | nucleic | GGCCTCAGTGAAGATTTCCTGCAAGACATCTGGATACACCTTCA |
| | | acid | CCAACTACTATATGGTCTGGGTGCGACAGGCCCCTGGACAAGGA |
| | | sequence | CTTGAGTGGATGGGCATAATCAAACCTGGTGGTGGTAACACGAT |
| | | | CTACGCACAGAAATTCCAGGGCAGAGTCACCATGACCAGGGACA |
| | | | CGTCCACGAACACAGTCTATTTGGACCTGAACAGTCTGCGATCT |
| | | | GAGGACACGGCCGTGTATTATTGTGCGAGAACTGGTGATAGGGC |
| | | | TTTTGATGTCTGGGGCCACGGGACAGTGGTCACCGTCTCTTCA |
| 2085 | YANG-2290 | VH domain | QMQLVQSGTEVKKPGASVKISCKTSGYTFTNYYMVWVRQAPGQG |
| | | amino | LEWMGIIKPGGGNTIYAQKFQGRVTMTRDTSTNTVYLDLNSLRS |
| | | acid | EDTAVYYCARTGDRAFDVWGHGTVVTVSS |
| | | sequence | |
| 2086 | YANG-2290 | HCDR1 | GYTFTNYY |
| 2087 | YANG-2290 | HCDR2 | IKPGGGNT |
| 2088 | YANG-2290 | HCDR3 | ARTGDRAFDV |
| 2089 | YANG-2290 | VL domain | CAGTCTGTGTTGACGCAGCCGCCCTCAGTGTCTGCGGCCCCAGG |
| | | nucleic | ACAGAAGGTCACCATGTCCTGCTCTGGAAGCAGCTCCAACATTG |
| | | acid | GGAATAATTATGTTTCCTGGTACCAGCAGTTCCCAGGAACAGCC |
| | | sequence | CCCAAACTCCTCATCTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGCTGACCGTCCTA |
| 2090 | YANG-2290 | VL domain | QSVLTQPPSVSAAPGQKVTMSCSGSSSNIGNNYVSWYQQFPGTA |
| | | amino | PKLLIYDNNKRPSGIPDRFSGSKSGTSATLGITGLQTGDEADYY |
| | | acid | CGTWDSSLGAGVFGGGTKLTVL |
| | | sequence | |
| 2091 | YANG-2290 | LCDR1 | SSNIGNNY |
| 2092 | YANG-2290 | LCDR2 | DNN |
| 2093 | YANG-2290 | LCDR3 | GTWDSSLGAGV |
| 2094 | YANG-2291 | VH domain | CAGATTCAGTTGGTGCAGTCTGGGGCTGAGGTGAAGAAGCCTGG |
| | | nucleic | GGCCTCAGTGAAGTTTTCCTGCAAGACATCTGGATACACCTTCA |
| | | acid | CCAGCTACTATATGGTCTGGGTGCGACAGGCCCCTGGACAAGGA |
| | | sequence | CTTGAGTGGATGGGCATAATCAAACCTAGTGGTGGTAACACAAT |
| | | | CTACGCACAGAAGTTCCAGGGCAGAGTCACCATGACCAGGGACA |
| | | | CGTCCACGAACACAGTCTACATGGACCTGAGCAGTCTGAGATCT |
| | | | GAGGACACGGCCGTGTATTTTTGTGCGAGAACTGGGGATAGGGC |
| | | | TTTTGATGTCTGGGGCCACGGGACAATGATCACCGTCTCTTCA |
| 2095 | YANG-2291 | VH domain | QIQLVQSGAEVKKPGASVKFSCKTSGYTFTSYYMVWVRQAPGQG |
| | | amino | LEWMGIIKPSGGNTIYAQKFQGRVTMTRDTSTNTVYMDLSSLRS |
| | | acid | EDTAVYFCARTGDRAFDVWGHGTMITVSS |
| | | sequence | |
| 2096 | YANG-2291 | HCDR1 | GYTFTSYY |
| 2097 | YANG-2291 | HCDR2 | IKPSGGNT |
| 2098 | YANG-2291 | HCDR3 | ARTGDRAFDV |
| 2099 | YANG-2291 | VL domain | CAGTCTGTATTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GAAATAATTTTGTATCCTGGTACCAACAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGCTGACCGTCCTA |
| 2100 | YANG-2291 | VL domain | QSVLTQPPSVSAAPGQKVTISCSGSSSNIGNNFVSWYQQFPGTA |
| | | amino | PKLLIYDNNKRPSGIPDRESGSKSGTSATLGITGLQTGDEADYY |
| | | acid | CGTWDSSLGAGVFGGGTKLTVL |
| | | sequence | |
| 2101 | YANG-2291 | LCDR1 | SSNIGNNF |
| 2102 | YANG-2291 | LCDR2 | DNN |
| 2103 | YANG-2291 | LCDR3 | GTWDSSLGAGV |
| 2104 | YANG-2292 | VH domain | CAGATGCAATTGGTGCAGTCTGGGGCTGAGGTGAAGAAGCCTGG |
| | | nucleic | GGCCTCAGTGAAGATCTCCTGCAAGACATCTGGATACACCTTCA |
| | | acid | TCAACTATTATATGGTCTGGGTGCGACAGGCCCCGGGACAAGGA |
| | | sequence | CTTGAGTGGATGGGCATAATCAAACCCAGTGGTGGTAACACGAT |
| | | | CTACGCACAGAAGTTCCAGGGCAGAGTCACCATGACCAGGGACA |
| | | | CGTCCACGAACACAGTCTATTTGGACCTGAGCAGTCTGAGATCT |
| | | | GAGGACACGGCCGTGTATTATTGTGCGAGAACTGGAGATAGGGC |
| | | | TTTTGATGTCTGGGGCCACGGGACAATGGTCACCGTCTCTTCA |
| 2105 | YANG-2292 | VH domain | QMQLVQSGAEVKKPGASVKISCKTSGYTFINYYMVWVRQAPGQG |
| | | amino | LEWMGIIKPSGGNTIYAQKFQGRVTMTRDTSTNTVYLDLSSLRS |
| | | acid | EDTAVYYCARTGDRAFDVWGHGTMVTVSS |
| | | sequence | |
| 2106 | YANG-2292 | HCDR1 | GYTFINYY |
| 2107 | YANG-2292 | HCDR2 | IKPSGGNT |
| 2108 | YANG-2292 | HCDR3 | ARTGDRAFDV |
| 2109 | YANG-2292 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTATGTTTCCTGGTACCAGCAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGCTGACCGTCCTA |
| 2110 | YANG-2292 | VL domain | QSVLTQPPSVSAAPGQKVTISCSGSSSNIGNNYVSWYQQFPGTA |
| | | amino | PKLLIYDNNKRPSGIPDRESGSKSGTSATLGITGLQTGDEADYY |
| | | acid | CGTWDSSLGAGVFGGGTKLTVL |
| | | sequence | |
| 2111 | YANG-2292 | LCDR1 | SSNIGNNY |
| 2112 | YANG-2292 | LCDR2 | DNN |
| 2113 | YANG-2292 | LCDR3 | GTWDSSLGAGV |
| 2114 | YANG-2293 | VH domain | CAGATGCAATTGCTGCAGTCTGGGGCTGAGGTGAAGAAGCCTGG |
| | | nucleic | GGCCTCAGCGAAGATTTCCTGCAAGACATCTGGATATACCTTCA |
| | | acid | CCAACTACTATATGGTCTGGGTGCGACAGGCTCCTGGACAAGGA |
| | | sequence | CTTGAGTGGATGGGCATAATCAAACCTAGTGGTGGTAACACGAT |
| | | | CTACGCACAGAAGTGCCAGGGCAGAGTCACCATGACCAGGGACA |
| | | | CGTCCACGAACACAGTCTATTTGGACCTGAACAGTCTGAGATCT |
| | | | GAGGACACGGCCGTTTATTATTGTGCGAGAACTGGGGATAGGGC |
| | | | TTTTGATTTCTGGGGCCACGGGACAATGGTCACCGTCTCTTCA |
| 2115 | YANG-2293 | VH domain | QMQLLQSGAEVKKPGASAKISCKTSGYTFTNYYMVWVRQAPGQG |
| | | amino | LEWMGIIKPSGGNTIYAQKCQGRVTMTRDTSTNTVYLDLNSLRS |
| | | acid | EDTAVYYCARTGDRAFDFWGHGTMVTVSS |
| | | sequence | |
| 2116 | YANG-2293 | HCDR1 | GYTFTNYY |
| 2117 | YANG-2293 | HCDR2 | IKPSGGNT |
| 2118 | YANG-2293 | HCDR3 | ARTGDRAFDE |
| 2119 | YANG-2293 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTATGTTTCCTGGTATCACCAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAT |
| | | | TGCGGAACATGGGATAGCAGCCTGGGTGCTGGGGTGTTCGGCGG |
| | | | AGGGACCAAGCTGACCGTCCTA |
| 2120 | YANG-2293 | VL domain | QSVLTQPPSVSAAPGQKVTISCSGSSSNIGNNYVSWYHQFPGTA |
| | | amino | PKLLIYDNNKRPSGIPDRFSGSKSGTSATLGITGLQTGDEADYY |
| | | acid | CGTWDSSLGAGVFGGGTKLTVL |
| | | sequence | |
| 2121 | YANG-2293 | LCDR1 | SSNIGNNY |
| 2122 | YANG-2293 | LCDR2 | DNN |
| 2123 | YANG-2293 | LCDR3 | GTWDSSLGAGV |
| 2124 | YANG-2294 | VH domain | CAGATTCAGTTGGTGCAGTCTGGGGCTGAGGCGAAGAAGCCAGG |
| | | nucleic | GGCCCCAGTGAAGTTTTCTGGCAAGACATCTGGATACAACTTCA |
| | | acid | CCAACTACTACATGGNCTGTGTGCGACAGGCCCCTGAACAGGGA |
| | | sequence | CTTTAGTGGATGGGCATAATTAAACCTAGCGGTGGTAACACAAT |
| | | | CTCCGCACAGAAGTACCAGGGCAGAGTCACCATGACCAGGGACA |
| | | | CGTCCACGAACACAGTCTACATGGACCAGAACAGTCTGAGATCT |
| | | | GAGGACACGGCCGTCTATTATTGTGCGAGAACTGGAGATAGGGC |
| | | | TTTTGATGTCTGGGGCCACGGGACAATGGTCACCGTGTCTTCA |
| | | | (N = unknown base) |
| 2125 | YANG-2294 | VH domain | QIQLVQSGAEAKKPGAPVKFSGKTSGYNFTNYYMXCVRQAPEQG |
| | | amino | LXWMGIIKPSGGNTISAQKYQGRVTMTRDTSTNTVYMDQNSLRS |
| | | acid | EDTAVYYCARTGDRAFDVWGHGTMVTVSS |
| | | sequence | (X = unknown amino acid) |
| 2126 | YANG-2294 | HCDR1 | GYNFTNYY |
| 2127 | YANG-2294 | HCDR2 | IKPSGGNT |
| 2128 | YANG-2294 | HCDR3 | ARTGDRAFDV |
| 2129 | YANG-2294 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTATGTATCCTGGTACCAGCAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGGCTCCAGACTGGGGACGAGGCCGATTATTAC |
| | | | TGCGGAACATGGGATAGCAGCCTGGGTGCTGGGGTGTTCGGCGG |
| | | | AGGGACCAAACTGACCGTCCTA |
| 2130 | YANG-2294 | VL domain | QSVLTQPPSVSAAPGQKVTISCSGSSSNIGNNYVSWYQQFPGTA |
| | | amino | PKLLIYDNNKRPSGIPDRFSGSKSGTSATLGITGLQTGDEADYY |
| | | acid | CGTWDSSLGAGVFGGGTKLTVL |
| | | sequence | |
| 2131 | YANG-2294 | LCDR1 | SSNIGNNY |
| 2132 | YANG-2294 | LCDR2 | DNN |
| 2133 | YANG-2294 | LCDR3 | GTWDSSLGAGV |
| 2134 | YANG-2295 | VH domain | CAGATGCAGCTGGTGCAGTCTGGGGCTGAGGTGAAGAAGCCTGG |
| | | nucleic | GGCCTCAGTGAGGTTTTCCTGCAAGACATCTGGATACACCTTCA |
| | | acid | CCAGTTACTATATGGTCTGGGTGCGACAGGCCCCTGGACAAGGA |
| | | sequence | CTTGAATGGATGGGCATAATCAAACCTAGTGGTGGTAACACAAT |
| | | | CTACGCACAGAAGTTCCAGGGCAGAGTCACCATGACCAGGGACA |
| | | | CGTCCACGAATACAGTCTACATGGACCTGAGGAGTCTGAGCTCT |
| | | | GAGGACACGGCCGTGTATTATTGTGCGAGAACTGGGGATAGGGC |
| | | | TTTTGATGTCTGGGGCCAAGGGACAATGGTCACCGTCTCTTCA |
| 2135 | YANG-2295 | VH domain | QMQLVQSGAEVKKPGASVRFSCKTSGYTFTSYYMVWVRQAPGQG |
| | | amino | LEWMGIIKPSGGNTIYAQKFQGRVTMTRDTSTNTVYMDLRSLSS |
| | | acid | EDTAVYYCARTGDRAFDVWGQGTMVTVSS |
| | | sequence | |
| 2136 | YANG-2295 | HCDR1 | GYTFTSYY |
| 2137 | YANG-2295 | HCDR2 | IKPSGGNT |
| 2138 | YANG-2295 | HCDR3 | ARTGDRAFDV |
| 2139 | YANG-2295 | VL domain | CAGTCTGTGTTGACGCAGCCGCCCTCAGTGTCTGCGGTCCCAGG |
| | | nucleic | ACAGAAGGTCACCATCTCCTGCTCTGGAAGCAGCTCCAACATTG |
| | | acid | GGAATAATTTTGTATCCTGGTACCTGCAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGCCACGTCAGCCACCC |
| | | | TGGGCATCACCGGACTCCAGACTGGGGACGAGGCCGATTATTAT |
| | | | TGCGGAACATGGGACAGCAGCCTGGGTGCTGGGGTGTTCGGCGG |
| | | | AGGGACCCAGCTGACCGTCCTA |
| 2140 | YANG-2295 | VL domain | QSVLTQPPSVSAVPGQKVTISCSGSSSNIGNNFVSWYLQFPGTA |
| | | amino | PKLLIYDNNKRPSGIPDRESGSKSATSATLGITGLQTGDEADYY |
| | | acid | CGTWDSSLGAGVFGGGTQLTVL |
| | | sequence | |
| 2141 | YANG-2295 | LCDR1 | SSNIGNNF |
| 2142 | YANG-2295 | LCDR2 | DNN |
| 2143 | YANG-2295 | LCDR3 | GTWDSSLGAGV |
| 2144 | YANG-2296 | VH domain | CAGATTCAGTTGGTGCAGTCTGGGGCTGAGGTGAAGAAGCCTGG |
| | | nucleic | GGCCTCAGTGAAGTTTTCCTGCAAGACATCTGGATACACCTTCA |
| | | acid | CCAGCTACTATATGGTCTGGGTGCGACAGGCCCCTGGACAAGGA |
| | | sequence | CTTGAGTGGATGGGCATAATCAAACCTAGTGGTGGTAACACAAT |
| | | | CTACGCACAGAAGTTCCAGGGCAGAGTCACCATGACCAGGGACA |
| | | | CGTCCACGAACACAGTCTACATGGACCTGAACAGTCTGAGATCT |
| | | | GAAGACACGGCCGTGTATTATTGTGCGAGAACTGGGGATAGGGC |
| | | | TTTTGATGTCTGGGGCCACGGGACAATGATCACCGTCTCTTCA |
| 2145 | YANG-2296 | VH domain | QIQLVQSGAEVKKPGASVKFSCKTSGYTFTSYYMVWVRQAPGQG |
| | | amino | LEWMGIIKPSGGNTIYAQKFQGRVTMTRDTSTNTVYMDLNSLRS |
| | | acid | EDTAVYYCARTGDRAFDVWGHGTMITVSS |
| | | sequence | |
| 2146 | YANG-2296 | HCDR1 | GYTFTSYY |
| 2147 | YANG-2296 | HCDR2 | IKPSGGNT |
| 2148 | YANG-2296 | HCDR3 | ARTGDRAFDV |
| 2149 | YANG-2296 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTTTGTATCCTGGTACCAACAGTTTCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGCTGACCGTCCTA |
| 2150 | YANG-2296 | VL domain | QSVLTQPPSVSAAPGQKVTISCSGSSSNIGNNFVSWYQQFPGTA |
| | | amino | PKLLIYDNNKRPSGIPDRFSGSKSGTSATLGITGLQTGDEADYY |
| | | acid | CGTWDSSLGAGVFGGGTKLTVL |
| | | sequence | |
| 2151 | YANG-2296 | LCDR1 | SSNIGNNF |
| 2152 | YANG-2296 | LCDR2 | DNN |
| 2153 | YANG-2296 | LCDR3 | GTWDSSLGAGV |
| 2154 | YANG-2297 | VH domain | CAGATTCAGTTGGTGCAGTCTGGGGCTGAGGTGAAGAAGCCTGG |
| | | nucleic | GGCCTCAGTGAAGTTTTCCTGCAAGACATCTGGATACACCTTCA |
| | | acid | CCAACTACTATATGGTCTGGGTGCGACAGGCCCCTGGACAAGGA |
| | | sequence | CTTGAGTGGATGGGCATAATCAAACCTAGTGGTGGTAACACAAT |
| | | | CTACGCACAGAAGTTCCAGGGCAGAGTCACCATGACCAGGGACA |
| | | | CGTCCACGAACACAGTCTACATGGACCTGAACAGTCTGAGATCT |
| | | | GAGGACACGGCCGTCTATTATTGTGCGAGAACTGGAGATAGGGC |
| | | | TTTTGATGTCTGGGGCCACGGGACAATGGTCACCGTGTCTTCA |
| 2155 | YANG-2297 | VH domain | QIQLVQSGAEVKKPGASVKFSCKTSGYTFTNYYMVWVRQAPGQG |
| | | amino | LEWMGIIKPSGGNTIYAQKFQGRVTMTRDTSTNTVYMDLNSLRS |
| | | acid | EDTAVYYCARTGDRAFDVWGHGTMVTVSS |
| | | sequence | |
| 2156 | YANG-2297 | HCDR1 | GYTFTNYY |
| 2157 | YANG-2297 | HCDR2 | IKPSGGNT |
| 2158 | YANG-2297 | HCDR3 | ARTGDRAFDV |
| 2159 | YANG-2297 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTATGTATCCTGGTACCAGCAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAACTGACCGTCCTA |
| 2160 | YANG-2297 | VL domain | QSVLTQPPSVSAAPGQKVTISCSGSSSNIGNNYVSWYQQFPGTA |
| | | amino | PKLLIYDNNKRPSGIPDRFSGSKSGTSATLGITGLQTGDEADYY |
| | | acid | CGTWDSSLGAGVFGGGTKLTVL |
| | | sequence | |
| 2161 | YANG-2297 | LCDR1 | SSNIGNNY |
| 2162 | YANG-2297 | LCDR2 | DNN |
| 2163 | YANG-2297 | LCDR3 | GTWDSSLGAGV |
| 2164 | YANG-2298 | VH domain | CAGATGAAGTTGGTGCAGTCTGGGGCTGAGGTGAAGAAGCCTGG |
| | | nucleic | GGCCTCAGTGAAGATTTCCTGTAAGACATCTGGATACACCTTCA |
| | | acid | TCAACTACTATATGGTCTGGGTGCGACAGGCCCCTGGACAAGGA |
| | | sequence | CTTGAGTGGATGGGCATAATCAAACCTAGTGGTGGTAATACGAT |
| | | | CTACGCACAGAAGTTCCAGGGCAGAGTCACCATGACCAGGGACA |
| | | | CGTCCACGAACACAGTCTATATGGACCTGAGCAGTCTGAGATCT |
| | | | GAGGACACGGCCGTGTATTATTGTGCGAGAACTGGAGATAGGGC |
| | | | TTTTGATGTCTGGGGCCACGGGACAATGGTCACCGTCTCTTCA |
| 2165 | YANG-2298 | VH domain | QMKLVQSGAEVKKPGASVKISCKTSGYTFINYYMVWVRQAPGQG |
| | | amino | LEWMGIIKPSGGNTIYAQKFQGRVTMTRDTSTNTVYMDLSSLRS |
| | | acid | EDTAVYYCARTGDRAFDVWGHGTMVTVSS |
| | | sequence | |
| 2166 | YANG-2298 | HCDR1 | GYTFINYY |
| 2167 | YANG-2298 | HCDR2 | IKPSGGNT |
| 2168 | YANG-2298 | HCDR3 | ARTGDRAFDV |
| 2169 | YANG-2298 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAACTCCAACATTG |
| | | acid | GGAATAATTATGTTTCCTGGTACCAGCAGTTCCCAGGAACAGCC |
| | | sequence | CCCAAACTCCTCATTTATGACAATCATAAGCGACCCTCAGGGAT |
| | | | TCCTGACCGATTCTCTGCCTCCAAGTCTGGCACGTCAGCCACCC |
| | | | TGGGCATCACCGGACTCCAGACTGGGGACGAGGCCGATTATTAC |
| | | | TGCGGAACATGGGATAGCGGCCTGGGTGCTGGGGTGTTCGGCGG |
| | | | AGGGACCAAGCTGACCGTCCTA |
| 2170 | YANG-2298 | VL domain | QSVLTQPPSVSAAPGQKVTISCSGSNSNIGNNYVSWYQQFPGTA |
| | | amino | PKLLIYDNHKRPSGIPDRFSASKSGTSATLGITGLQTGDEADYY |
| | | acid | CGTWDSGLGAGVFGGGTKLTVL |
| | | sequence | |
| 2171 | YANG-2298 | LCDR1 | NSNIGNNY |
| 2172 | YANG-2298 | LCDR2 | DNH |
| 2173 | YANG-2298 | LCDR3 | GTWDSGLGAGV |
| 2174 | YANG-2299 | VH domain | CAGATGCAGTTGGTGCAGTCTGGGGCTGAGGTGAAGAAGCCTGG |
| | | nucleic | GGCCTCAGTGAAGTTTTCCTGCAAGACATCTGGATACACCTTCA |
| | | acid | CCAATTATTATATGGTCTGGGTGCGACAGGCCCCTGGACAAGGA |
| | | sequence | CTTGAATGGATGGGCATAATCAAACCTAGTGGTGGTAACACAAT |
| | | | CTACGCACAGAAGTTCCAGGGCAGAGTCACCATGACCAGGGACA |
| | | | CGTCCACGAATACAGTCTATATGGACCTGAGGAGTCTGAGATCT |
| | | | GAGGACACGGCCGTGTATTATTGTGCGAGAACTGGGGATAGGGC |
| | | | TTTTGATGTCTGGGGCCAAGGGACAATGGTCACCGTCTCTTCA |
| 2175 | YANG-2299 | VH domain | QMQLVQSGAEVKKPGASVKFSCKTSGYTFTNYYMVWVRQAPGQG |
| | | amino | LEWMGIIKPSGGNTIYAQKFQGRVTMTRDTSTNTVYMDLRSLRS |
| | | acid | EDTAVYYCARTGDRAFDVWGQGTMVTVSS |
| | | sequence | |
| 2176 | YANG-2299 | HCDR1 | GYTFTNYY |
| 2177 | YANG-2299 | HCDR2 | IKPSGGNT |
| 2178 | YANG-2299 | HCDR3 | ARTGDRAFDV |
| 2179 | YANG-2299 | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGTTCCAATATTG |
| | | acid | GGAATAATTTTGTATCCTGGTACCTGCAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCCAGCTGACCGTCCTA |
| 2180 | YANG-2299 | VL domain | QSVLTQPPSVSAAPGQKVTISCSGSSSNIGNNFVSWYLQFPGTA |
| | | amino | PKLLIYDNNKRPSGIPDRFSGSKSGTSATLGITGLQTGDEADYY |
| | | acid | CGTWDSSLGAGVFGGGTQLTVL |
| | | sequence | |
| 2181 | YANG-2299 | LCDR1 | SSNIGNNF |
| 2182 | YANG-2299 | LCDR2 | DNN |
| 2183 | YANG-2299 | LCDR3 | GTWDSSLGAGV |
| 2184 | YANG-2299a | VH domain | CAGATGCAGCTGGTGCAGTCTGGGGCTGAGGTGAAGAAGCCTGG |
| | | nucleic | GGCCTCAGTGAAGTTTTCCTGCAAGACATCTGGATACACCTTCA |
| | | acid | CCAGCTACTATATGGTCTGGGTGCGACAGGCCCCTGGACAAGGA |
| | | sequence | CTTGAGTGGATGGGCATAATCAAACCTAGTGGTGGTAACACAAT |
| | | | CTATGCACGGAAGTTCCAGGGCAGAGTCACCATGACCAGGGACA |
| | | | CGTCCACGAACACAGTCTACATGGACCTGAGCAGTCTGAGATCT |
| | | | GAGGACACGGCCGTGTATTATTGTGCGAGAACTGGGGATAGGGC |
| | | | TTTTGATGTCTGGGGCCAAGGGACATTGGTCACCGTCTCTTCA |
| 2185 | YANG-2299a | VH domain | QMQLVQSGAEVKKPGASVKFSCKTSGYTFTSYYMVWVRQAPGQG |
| | | amino | LEWMGIIKPSGGNTIYARKFQGRVTMTRDTSTNTVYMDLSSLRS |
| | | acid | EDTAVYYCARTGDRAFDVWGQGTLVTVSS |
| | | sequence | |
| 2186 | YANG-2299a | HCDR1 | GYTFTSYY |
| 2187 | YANG-2299a | HCDR2 | IKPSGGNT |
| 2188 | YANG-2299a | HCDR3 | ARTGDRAFDV |
| 2189 | YANG-2299a | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAGTAATTATGTCTCCTGGTACCAGCAGCTCCCAGGAACAGCC |
| | | sequence | CCCAAACTCCTCATTTATGACAATAATAAGCGCCCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAACTGACCGTCCTA |
| 2190 | YANG-2299a | VL domain | QSVLTQPPSVSAAPGQKVTISCSGSSSNIGSNYVSWYQQLPGTA |
| | | amino | PKLLIYDNNKRPSGIPDRFSGSKSGTSATLGITGLQTGDEADYY |
| | | acid | CGTWDSSLGAGVFGGGTKLTVL |
| | | sequence | |
| 2191 | YANG-2299a | LCDR1 | SSNIGSNY |
| 2192 | YANG-2299a | LCDR2 | DNN |
| 2193 | YANG-2299a | LCDR3 | GTWDSSLGAGV |
| 2194 | YANG-2299b | VH domain | CAGATACAACTGGTGCAGTCTGGGGCTGAGGTGAAGAAGCCTGG |
| | | nucleic | GGCCTCAGTGAAGTTTTCCTGCAAGACATCTGGATACACCTTCA |
| | | acid | CCAACTACTATATGGTCTGGGTGCGACAGGCCCCTGGACAAGGT |
| | | sequence | CTTGAGTGGATGGGCATAATCAAACCTAGTGGTGGTAACACAAT |
| | | | CTACGCACAGAACTTCCAGGGCAGAGTCACCATGACCAGGGACA |
| | | | CGTCCACGAACACAGTCTACATGGACCTGAGGAGTCTGAGATCT |
| | | | GGGGACACGGCCGTGTATTATTGTGCGAGAAGTGGTGATAGGGC |
| | | | TTTTGATGTCTGGGGCCAAGGGACAATGGTCACCGTCTCTTCA |
| 2195 | YANG-2299b | VH domain | QIQLVQSGAEVKKPGASVKFSCKTSGYTFTNYYMVWVRQAPGQG |
| | | amino | LEWMGIIKPSGGNTIYAQNFQGRVTMTRDTSTNTVYMDLRSLRS |
| | | acid | GDTAVYYCARSGDRAFDVWGQGTMVTVSS |
| | | sequence | |
| 2196 | YANG-2299b | HCDR1 | GYTFTNYY |
| 2197 | YANG-2299b | HCDR2 | IKPSGGNT |
| 2198 | YANG-2299b | HCDR3 | ARSGDRAFDV |
| 2199 | YANG-2299b | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTTTGTATCCTGGTACCAGCAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGCTGACCGTCCTA |
| 2200 | YANG-2299b | VL domain | QSVLTQPPSVSAAPGQKVTISCSGSSSNIGNNFVSWYQQFPGTA |
| | | amino | PKLLIYDNNKRPSGIPDRFSGSKSGTSATLGITGLQTGDEADYY |
| | | acid | CGTWDSSLGAGVFGGGTKLTVL |
| | | sequence | |
| 2201 | YANG-2299b | LCDR1 | SSNIGNNF |
| 2202 | YANG-2299b | LCDR2 | DNN |
| 2203 | YANG-2299b | LCDR3 | GTWDSSLGAGV |
| 2204 | YANG-2299c | VH domain | CAGATACAGCTGGTGCAGTCTGGGGCTGAGGTGAAGAAGCCTGG |
| | | nucleic | GGCCTCAGTGAGGCTTTCCTGCAAGACATCTGGATACACCTTCA |
| | | acid | CCAACTACTATATGGTCTGGGTGCGACAGGCCCCTGGACAAGGA |
| | | sequence | CTTGAGTGGATGGGCATAATCAAACCGAGTGGTGGTAATACAAT |
| | | | CTACGCACAGAACTTCCAGGGCAGAGTCACCATGACCAGGGACA |
| | | | CGTCCACGAGAACAGTCTACATGGACCTGAGGAGTCTGAGATCT |
| | | | GAGGACACGGCCGTGTATTATTGTGCGAGAACTGGTGATAGGGC |
| | | | TTTTGATGTCTGGGGCCAAGGGACAATGGTCACCGTCTCTTTA |
| 2205 | YANG-2299c | VH domain | QIQLVQSGAEVKKPGASVRLSCKTSGYTFTNYYMVWVRQAPGQG |
| | | amino | LEWMGIIKPSGGNTIYAQNFQGRVTMTRDTSTRTVYMDLRSLRS |
| | | acid | EDTAVYYCARTGDRAFDVWGQGTMVTVSL |
| | | sequence | |
| 2206 | YANG-2299c | HCDR1 | GYTFTNYY |
| 2207 | YANG-2299c | HCDR2 | IKPSGGNT |
| 2208 | YANG-2299c | HCDR3 | ARTGDRAFDV |
| 2209 | YANG-2299c | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTTTGTATCCTGGTACCAGCAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGAATATTAC |
| | | | TGCGGAACATGGGATAGCAGCCTGGGTGCTGGGGTGTTCGGCGG |
| | | | AGGGACCAAGCTGACCGTCCTA |
| 2210 | YANG-2299c | VL domain | QSVLTQPPSVSAAPGQKVTISCSGSSSNIGNNFVSWYQQFPGTA |
| | | amino | PKLLIYDNNKRPSGIPDRFSGSKSGTSATLGITGLQTGDEAEYY |
| | | acid | CGTWDSSLGAGVFGGGTKLTVL |
| | | sequence | |
| 2211 | YANG-2299c | LCDR1 | SSNIGNNF |
| 2212 | YANG-2299c | LCDR2 | DNN |
| 2213 | YANG-2299c | LCDR3 | GTWDSSLGAGV |
| 2214 | YANG-2299d | VH domain | CAGATGCAATTGGTGCAGTCTGGGGCTGAGGTGAAGAAGCCTGG |
| | | nucleic | GGCCTCAGTGAAGATTTCCTGCAAGACATCTGGATACACCTTCA |
| | | acid | CCAACTATTATATGGTCTGGGTGCGACAGGCCCCTGGACAAGGA |
| | | sequence | CTTGAGTGGATGGGCATAATCAAACCTAGTGGTGGTAACACGAT |
| | | | CTACGCACAGAAGTTCCAGGGCAGAGTCACCATGACCAGGGACA |
| | | | CGTCCACGAACACAGTCTATTTGGACCTGAGCAGTCTGAGATCT |
| | | | GAGGACACGGCCGTGTATTATTGTGCGAGAACTGGTGATAGGGC |
| | | | TTTTGATGTCTGGGGCCACGGGACAGTGGTCACCGTCTCTTCA |
| 2215 | YANG-2299d | VH domain | QMQLVQSGAEVKKPGASVKISCKTSGYTFTNYYMVWVRQAPGQG |
| | | amino | LEWMGIIKPSGGNTIYAQKFQGRVTMTRDTSTNTVYLDLSSLRS |
| | | acid | EDTAVYYCARTGDRAFDVWGHGTVVTVSS |
| | | sequence | |
| 2216 | YANG-2299d | HCDR1 | GYTFTNYY |
| 2217 | YANG-2299d | HCDR2 | IKPSGGNT |
| 2218 | YANG-2299d | HCDR3 | ARTGDRAFDV |
| 2219 | YANG-2299d | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTATGTTTCCTGGTACCAGCAGTTCCCAGGAACAGCC |
| | | sequence | CCCAAACTCCTCATCTATGACAATAATAAGCGACCCTCAGGGAT |
| | | | TCCTGACCGATTCTCTGGCTCCAAGTCTGGCACGTCAGCCACCC |
| | | | TGGCCATCACCGGACTCCAGACTGGGGACGAGGCCGATTATTAC |
| | | | TGCGGAACATGGGATAGCAGCCTGGGTGTTGGGGTGTTCGGCGG |
| | | | AGGGACCAAACTGACCGTCCTA |
| 2220 | YANG-2299d | VL domain | QSVLTQPPSVSAAPGQKVTISCSGSSSNIGNNYVSWYQQFPGTA |
| | | amino | PKLLIYDNNKRPSGIPDRFSGSKSGTSATLAITGLQTGDEADYY |
| | | acid | CGTWDSSLGVGVFGGGTKLTVL |
| | | sequence | |
| 2221 | YANG-2299d | LCDR1 | SSNIGNNY |
| 2222 | YANG-2299d | LCDR2 | DNN |
| 2223 | YANG-2299d | LCDR3 | GTWDSSLGVGV |
| 2224 | YANG-2299e | VH domain | CAGATGCAGCTGGTACAGTCTGGGGCTGAGGTGAAGAAGCCTGG |
| | | nucleic | GGCCTCAGTGAAGTTTTCCTGCAAGACATCTGGATACACCTTCA |
| | | acid | CCAGCTATTATATGGTCTGGGTGCGACAGGCCCCTGGACAAGGA |
| | | sequence | CTTGAGTGGATGGGCATAATCAAACCTAGTGGTGGTAACACAAT |
| | | | CTATGCACAGAAGTTCCAGGGCAGAGTCACCATGACCAGGGACA |
| | | | CGTCCACGAACACAGTCTACATGGACCTGAGTAGTCTGAGATCT |
| | | | GAGGACACGGCCGTGTATTATTGTGCGAGAACTGGGGATAGGGC |
| | | | TTTTGATGTCTGGGGCCAAGGGACAATGGTCACCGTCTCTTCA |
| 2225 | YANG-2299e | VH domain | QMQLVQSGAEVKKPGASVKFSCKTSGYTFTSYYMVWVRQAPGQG |
| | | amino | LEWMGIIKPSGGNTIYAQKFQGRVTMTRDTSTNTVYMDLSSLRS |
| | | acid | EDTAVYYCARTGDRAFDVWGQGTMVTVSS |
| | | sequence | |
| 2226 | YANG-2299e | HCDR1 | GYTFTSYY |
| 2227 | YANG-2299e | HCDR2 | IKPSGGNT |
| 2228 | YANG-2299? | HCDR3 | ARTGDRAFDV |
| 2229 | YANG-2299? | VL domain | CAGTCTGTGTTGACGCAGCCGCCCTCAGTGTCTGCGGCCCCAGG |
| | | nucleic | ACAGAAGGTCACCATCTCCTGCTCTGGAAGCACCTCCAACATTG |
| | | acid | GGAGTAATTATGTCTCCTGGTACCAGCAGCTCCCAGGAACAGCC |
| | | sequence | CCCAAACTCCTCATTTATGACAATAATAAGCGCCCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAACTGACCGTCCTA |
| 2230 | YANG-2299? | VL domain | QSVLTQPPSVSAAPGQKVTISCSGSTSNIGSNYVSWYQQLPGTA |
| | | amino | PKLLIYDNNKRPSGIPDRFSGSKSGTSATLGITGLQTGDEADYY |
| | | acid | CGTWDSSLGAGVFGGGTKLTVL |
| | | sequence | |
| 2231 | YANG-2299e | LCDR1 | TSNIGSNY |
| 2232 | YANG-2299e | LCDR2 | DNN |
| 2233 | YANG-2299e | LCDR3 | GTWDSSLGAGV |
| 2234 | YANG-2299f | VH domain | CAGATACAGCTGGTGCAGTCTGGGGCTGAGGTGAAGAAGCCTGG |
| | | nucleic | GGCCTCAGTGAAGTTTTCCTGCAAGACATCTGGATACACCTTCA |
| | | acid | CCAACTACTATATGGTCTGGGTGCGACAGGCCCCTGGGCAAGGA |
| | | sequence | CTTGAGTGGATGGGCATAATCAAACCTAGCGGTGGTAACACAAT |
| | | | CTACGCACAGAACTTCCAGGGCAGAGTCACCATGACCAGGGACA |
| | | | CGTCCACGAACACAGTCTACATGGACCTGAGGAGTCTGAGATCT |
| | | | GAGGACACGGCCGTGTATTATTGTGCGAGAACTGGCGATAGGGC |
| | | | TTTTGATGTCTGGGGCCAAGGGACAATGGTCACCGTCTCTTCA |
| 2235 | YANG-2299f | VH domain | QIQLVQSGAEVKKPGASVKFSCKTSGYTFTNYYMVWVRQAPGQG |
| | | amino | LEWMGIIKPSGGNTIYAQNFQGRVTMTRDTSTNTVYMDLRSLRS |
| | | acid | EDTAVYYCARTGDRAFDVWGQGTMVTVSS |
| | | sequence | |
| 2236 | YANG-2299f | HCDR1 | GYTFTNYY |
| 2237 | YANG-2299f | HCDR2 | IKPSGGNT |
| 2238 | YANG-2299f | HCDR3 | ARTGDRAFDV |
| 2239 | YANG-2299f | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTTTGTATCCTGGTACCAGCAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGCTGACCGTCCTA |
| 2240 | YANG-2299f | VL domain | QSVLTQPPSVSAAPGQKVTISCSGSSSNIGNNFVSWYQQFPGTA |
| | | amino | PKLLIYDNNKRPSGIPDRFSGSKSGTSATLGITGLQTGDEADYY |
| | | acid | CGTWDSSLGAGVFGGGTKLTVL |
| | | sequence | |
| 2241 | YANG-2299f | LCDR1 | SSNIGNNF |
| 2242 | YANG-2299f | LCDR2 | DNN |
| 2243 | YANG-2299f | LCDR3 | GTWDSSLGAGV |
| 2244 | YANG-2299g | VH domain | CAGATACAACTGGTGCAGTCTGGGGCTGAGGTGAAGAAGCCTGG |
| | | nucleic | GGCCTCAGTGAAGTTTTCCTGCAAGACATCTGGATACACCTTCA |
| | | acid | CCAACTACTATATGGTGTGGGTGCGACAGGCCCCTGGACAAGGA |
| | | sequence | CTTGAGTGGATGGGCATAATCAAACCTAGTGGTGGTAACACAAT |
| | | | CTACGCACAGAACTTCCAGGGCAGAGTCACCATGACCAGGGACA |
| | | | CGTCCACGAACACAGTGTACTTGGACCTGAGGAGTCTGAGATCT |
| | | | GAGGACACGGCCGTGTATTATTGTGCGAGAACTGGTGATAGGGC |
| | | | TTTTGATGTCTGGGGCCAAGGGACAATGGTCACCGTCTCTTCA |
| 2245 | YANG-2299g | VH domain | QIQLVQSGAEVKKPGASVKFSCKTSGYTFTNYYMVWVRQAPGQG |
| | | amino | LEWMGIIKPSGGNTIYAQNFQGRVTMTRDTSTNTVYLDLRSLRS |
| | | acid | EDTAVYYCARTGDRAFDVWGQGTMVTVSS |
| | | sequence | |
| 2246 | YANG-2299g | HCDR1 | GYTFTNYY |
| 2247 | YANG-2299g | HCDR2 | IKPSGGNT |
| 2248 | YANG-2299g | HCDR3 | ARTGDRAFDV |
| 2249 | YANG-2299g | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTTTGTATCCTGGTACCAGCAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGCTGACCGTCCTA |
| 2250 | YANG-2299g | VL domain | QSVLTQPPSVSAAPGQKVTISCSGSSSNIGNNFVSWYQQFPGTA |
| | | amino | PKLLIYDNNKRPSGIPDRESGSKSGTSATLGITGLQTGDEADYY |
| | | acid | CGTWDSSLGAGVFGGGTKLTVL |
| | | sequence | |
| 2251 | YANG-2299g | LCDR1 | SSNIGNNF |
| 2252 | YANG-2299g | LCDR2 | DNN |
| 2253 | YANG-2299g | LCDR3 | GTWDSSLGAGV |
| 2254 | YANG-2299h | VH domain | CAGATGCAATTGCTGCAGTCTGGGGCTGAGGTGAAGAAGCCTGG |
| | | nucleic | GGCCTCAGTGAAGATTTCCTGCAAGACATCTGGATACACCTTCA |
| | | acid | CCAACTACTATATGGTCTGGGTGCGACAGGCCCCTGGACAAGGA |
| | | sequence | CTTAAGTGGATGGGCATAATCAAACCTAGTGGTGGTAACACGAT |
| | | | CTACGCACAGAACTTCCAGGGCAGAGTCACCATGACCAGGGACA |
| | | | CGTCCACAAACACAATCTATTTGGACCTGAGCAGTCTGAGATCT |
| | | | GAGGACACGGCCGTATATTATTGTGCGAGAACTGGGGATAGGGC |
| | | | TTTTGATGTCTGGGGCCACGGGACAATGGTCACCGTCTCTTCA |
| 2255 | YANG-2299h | VH domain | QMQLLQSGAEVKKPGASVKISCKTSGYTFTNYYMVWVRQAPGQG |
| | | amino | LKWMGIIKPSGGNTIYAQNFQGRVTMTRDTSTNTIYLDLSSLRS |
| | | acid | EDTAVYYCARTGDRAFDVWGHGTMVTVSS |
| | | sequence | |
| 2256 | YANG-2299h | HCDR1 | GYTFTNYY |
| 2257 | YANG-2299h | HCDR2 | IKPSGGNT |
| 2258 | YANG-2299h | HCDR3 | ARTGDRAFDV |
| 2259 | YANG-2299h | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTATGTTTCCTGGTATCAGCAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAT |
| | | | TGCGGAACATGGGATAGCAGCCTGGGTGCTGGGGTGTTCGGCGG |
| | | | AGGGACCAAACTGACCGTCCTA |
| 2260 | YANG-2299h | VL domain | QSVLTQPPSVSAAPGQKVTISCSGSSSNIGNNYVSWYQQFPGTA |
| | | amino | PKLLIYDNNKRPSGIPDRFSGSKSGTSATLGITGLQTGDEADYY |
| | | acid | CGTWDSSLGAGVFGGGTKLTVL |
| | | sequence | |
| 2261 | YANG-2299h | LCDR1 | SSNIGNNY |
| 2262 | YANG-2299h | LCDR2 | DNN |
| 2263 | YANG-2299h | LCDR3 | GTWDSSLGAGV |
| 2264 | YANG-2299i | VH domain | CAGATGCAGTTGGTGCAGTCTGGGGCTGAGGTGAAGAAGCCTGG |
| | | nucleic | GGCCTCAGTGAAGATTTCCTGCAAGACATCTGGATACACCTTCA |
| | | acid | CCAATTACTATATGGTCTGGGTGCGACAGGCCCCTGGACAAGGA |
| | | sequence | CTTGAGTGGATGGGCATAATCAAACCTAGTGGTGGTAACACGAT |
| | | | CTACGCACAGAAGTTCCAGGGCAGAGTCACCATGACCAGGGACA |
| | | | CGTCCACGAACACAGTCTATATGGACCTGAACAGTCTGAGATCT |
| | | | GAGGACACGGCCGTATATTATTGTGCGAGAACTGGGGATAGGGC |
| | | | TTTTGATGTCTGGGGCCACGGGACAATGGTCACCGTCTCTTCA |
| 2265 | YANG-2299i | VH domain | QMQLVQSGAEVKKPGASVKISCKTSGYTFTNYYMVWVRQAPGQG |
| | | amino | LEWMGIIKPSGGNTIYAQKFQGRVTMTRDTSTNTVYMDLNSLRS |
| | | acid | EDTAVYYCARTGDRAFDVWGHGTMVTVSS |
| | | sequence | |
| 2266 | YANG-2299i | HCDR1 | GYTFTNYY |
| 2267 | YANG-2299i | HCDR2 | IKPSGGNT |
| 2268 | YANG-2299i | HCDR3 | ARTGDRAFDV |
| 2269 | YANG-2299i | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAATATTG |
| | | acid | GAACTAATTCTCTTTCCTGGTACCAGCAGTTCCCAGGAACAGCC |
| | | sequence | CCCAAACTCCTCATTTATGACAATAATAAACGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGGTGACCGTCCTA |
| 2270 | YANG-2299i | VL domain | QSVLTQPPSVSAAPGQKVTISCSGSSSNIGTNSLSWYQQFPGTA |
| | | amino | PKLLIYDNNKRPSGIPDRFSGSKSGTSATLGITGLQTGDEADYY |
| | | acid | CGTWDSSLGAGVFGGGTKVTVL |
| | | sequence | |
| 2271 | YANG-2299i | LCDR1 | SSNIGTNS |
| 2272 | YANG-2299i | LCDR2 | DNN |
| 2273 | YANG-2299i | LCDR3 | GTWDSSLGAGV |
| 2274 | YANG-2299j | VH domain | CAGATTCAGTTGGTGCAGTCTGGGGCTGAGGTGAAGAAGCCTGG |
| | | nucleic | GGCCTCAGTGAAGTTTTCCTGCAAGACATCTGGATACACCTTCA |
| | | acid | CCGGCTACTATATGGTCTGGGTGCGACAGGCCCCTGGACAAGGA |
| | | sequence | CTTGAGTGGATGGGCATAATCAAACCTAGTGGTGGTGACACAAT |
| | | | CTACGCACAGAAGTTCCAGGGCAGAGTCACCATGACCAGGGACA |
| | | | CGTCCACGAACACAGTCTACATGGACCTGAACAGTCTGAGATCT |
| | | | GAGGACACGGCCGTCTATTATTGTGCGAGAACTGGAGATAGGGC |
| | | | TTTTGATGTCTGGGGCCACGGGACAATGGTCACCGTGTCTTCA |
| 2275 | YANG-2299j | VH domain | QIQLVQSGAEVKKPGASVKFSCKTSGYTFTGYYMVWVRQAPGQG |
| | | amino | LEWMGIIKPSGGDTIYAQKFQGRVTMTRDTSTNTVYMDLNSLRS |
| | | acid | EDTAVYYCARTGDRAFDVWGHGTMVTVSS |
| | | sequence | |
| 2276 | YANG-2299j | HCDR1 | GYTFTGYY |
| 2277 | YANG-2299j | HCDR2 | IKPSGGDT |
| 2278 | YANG-2299j | HCDR3 | ARTGDRAFDV |
| 2279 | YANG-2299j | VL domain | CAGTCTGTGTTGACGCAGCCGCCCTCAGTGTCTGCGGCCCCAGG |
| | | nucleic | ACAGAAGGTCACCATCTCCTGCTCTGGAAGCAACTCCAACATTG |
| | | acid | GGAATAATTATGTATCCTGGTACCAGCAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAACTGACCGTCCTA |
| 2280 | YANG-2299j | VL domain | QSVLTQPPSVSAAPGQKVTISCSGSNSNIGNNYVSWYQQFPGTA |
| | | amino | PKLLIYDNNKRPSGIPDRFSGSKSGTSATLGITGLQTGDEADYY |
| | | acid | CGTWDSSLGAGVFGGGTKLTVL |
| | | sequence | |
| 2281 | YANG-2299j | LCDR1 | NSNIGNNY |
| 2282 | YANG-2299j | LCDR2 | DNN |
| 2283 | YANG-2299j | LCDR3 | GTWDSSLGAGV |
| 2284 | YANG-2299k | VH domain | CAGATACAGCTGGTGCAGTCTGGGGCTGAGGTGAAGAAGCCTGG |
| | | nucleic | GGCCTCAGTGAAGTTTTCCTGCAAGACATCTGGATACACCTTCA |
| | | acid | CCAACTACTATATGGTCTGGGTGCGACAGGCCCCTGGACAAGGA |
| | | sequence | CTTGAGTGGATGGGCATAATCAAACCTAGTGGTGGTAACACAAT |
| | | | CTACGCACAGAGTTTCCAGGGCAGAGTCACCATGACCAGGGACA |
| | | | CGTCCACGAACACAGTCTACATGGACCTGAGGAGTCTGAGATCT |
| | | | GAGGACACGGCCGTGTATTATTGTGCGAGAACTGGTGATAGGGC |
| | | | TTTTGATGTCTGGGGCCAAGGGACAATGGTCACCGTCTCTTCA |
| 2285 | YANG-2299k | VH domain | QIQLVQSGAEVKKPGASVKFSCKTSGYTFTNYYMVWVRQAPGQG |
| | | amino | LEWMGIIKPSGGNTIYAQSFQGRVTMTRDTSTNTVYMDLRSLRS |
| | | acid | EDTAVYYCARTGDRAFDVWGQGTMVTVSS |
| | | sequence | |
| 2286 | YANG-2299k | HCDR1 | GYTFTNYY |
| 2287 | YANG-2299k | HCDR2 | IKPSGGNT |
| 2288 | YANG-2299k | HCDR3 | ARTGDRAFDV |
| 2289 | YANG-2299k | VL domain | CAGTCTGTGTTGACGCAGCCGCCCTCAGTGTCTGCGGCCCCAGG |
| | | nucleic | ACAGAAGGTCACCATCTCCTGCTCTGGAAGCAGCTCCAACATTG |
| | | acid | GGAATAATTTTGTATCCTGGTACCAGCAGTTCCCAGGAACAGCC |
| | | sequence | CCCAAACTCCTCATTTATGACAATAATAAGCGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTGCTGGGGTGTTCGGCGG |
| | | | AGGGACCAAGCTGACCGTCCTA |
| 2290 | YANG-2299k | VL domain | QSVLTQPPSVSAAPGQKVTISCSGSSSNIGNNFVSWYQQFPGTA |
| | | amino | PKLLIYDNNKRPSGIPDRFSGSKSGTSATLGITGLQTGDEADYY |
| | | acid | CGTWDSSLGAGVFGGGTKLTVL |
| | | sequence | |
| 2291 | YANG-2299k | LCDR1 | SSNIGNNF |
| 2292 | YANG-2299k | LCDR2 | DNN |
| 2293 | YANG-2299k | LCDR3 | GTWDSSLGAGV |
| 2294 | YANG-22991 | VH domain | CAGATACAGCTGGTGCAGTCTGGGGCTGAGGTGAAGAAGCCTGG |
| | | nucleic | GGCCTCAGTGAGGTTTTCCTGCAAGACATCTGGATACACCTTCA |
| | | acid | CCAACTACTATATGGTCTGGGTGCGACAGGCCCCTGGACAAGGG |
| | | sequence | CTTGAGTGGATGGGCATAATCAAACCTAGTGGTGGTGACACAAT |
| | | | CTACGCACAGAACTTCCAGGGCAGAGTCACCATGACCAGGGACA |
| | | | CGTCCACGAACTCAGTCTACATGGACCTGAGGAGTCTGAGATCT |
| | | | GAGGACACGGCCGTGTATTATTGTGCGAGAACTGGTGATAGGGC |
| | | | TTTTGATGTCTGGGGCCAAGGGACAATGGTCACCGTCTCTTCA |
| 2295 | YANG-22991 | VH domain | QIQLVQSGAEVKKPGASVRFSCKTSGYTFTNYYMVWVRQAPGQG |
| | | amino | LEWMGIIKPSGGDTIYAQNFQGRVTMTRDTSTNSVYMDLRSLRS |
| | | acid | EDTAVYYCARTGDRAFDVWGQGTMVTVSS |
| | | sequence | |
| 2296 | YANG-22991 | HCDR1 | GYTFTNYY |
| 2297 | YANG-22991 | HCDR2 | IKPSGGDT |
| 2298 | YANG-22991 | HCDR3 | ARTGDRAFDV |
| 2299 | YANG-22991 | VL domain | CAGTCTGTGTTGACGCAGCCGCCCTCAGTGTCTGCGGCCCCAGG |
| | | nucleic | ACAGAAGGTCACCATCTCCTGCTCTGGACGCAGCTCCAACATTG |
| | | acid | GGAATAATTTTGTATCCTGGTACCAACAGTTCCCAGGAACAGCC |
| | | sequence | CCCAAACTCCTCATTTATGACAATAATAAACGACCCTCAGGGAT |
| | | | TCCTGACCGATTCTCTGGCTCCAAGTCTGGCACGTCAGCCACCC |
| | | | TGGGCATCACCGGACTCCAGACTGGGGACGAGGCCGATTATTAC |
| | | | TGCGGAACATGGGATAGCAGCCTGGGTACTGGGGTGTTCGGCGG |
| | | | AGGGACCAAGCTGACCGTCCTA |
| 2300 | YANG-22991 | VL domain | QSVLTQPPSVSAAPGQKVTISCSGRSSNIGNNFVSWYQQFPGTA |
| | | amino | PKLLIYDNNKRPSGIPDRESGSKSGTSATLGITGLQTGDEADYY |
| | | acid | CGTWDSSLGTGVFGGGTKLTVL |
| | | sequence | |
| 2301 | YANG-22991 | LCDR1 | SSNIGNNF |
| 2302 | YANG-22991 | LCDR2 | DNN |
| 2303 | YANG-22991 | LCDR3 | GTWDSSLGTGV |
| 2304 | YANG-2301 | VH domain | CAGGTCCACCTGATACAATCTGGGGCTGAGGTGAAGAAGCCTGG |
| | | nucleic | GGCCTCAGTGAAGGTCTCCTGCAAGGTTTCCGGATACACCCTCA |
| | | acid | TTGAAGTATCCGTGCACTGGGTGCGACAGGCTCCTGGAAAAGGG |
| | | sequence | CTTGAGTGGATGGGAGGTTTTGATCCTGAAGCAGCTGAAACAAT |
| | | | CTACGCACAGAAATTCCAGGGCAGAGTCACCATGACCGAGGACA |
| | | | CATCTACAGACACAGCCTACATGGACCTGAGCAGCCTGAAATCT |
| | | | GAAGACTCGGCCGTATATTACTGTGCAATAGGACCAGCAGTGAC |
| | | | TGGTAGAAACTCCGGGGAATACTATTACTATTACGGTATGGACG |
| | | | TCTGGGGCCAAGGGACCACGGTCACCGTCGCCTCA |
| 2305 | YANG-2301 | VH domain | QVHLIQSGAEVKKPGASVKVSCKVSGYTLIEVSVHWVRQAPGKG |
| | | amino | LEWMGGFDPEAAETIYAQKFQGRVTMTEDTSTDTAYMDLSSLKS |
| | | acid | EDSAVYYCAIGPAVTGRNSGEYYYYYGMDVWGQGTTVTVAS |
| | | sequence | |
| 2306 | YANG-2301 | HCDR1 | GYTLIEVS |
| 2307 | YANG-2301 | HCDR2 | FDPEAAET |
| 2308 | YANG-2301 | HCDR3 | AIGPAVTGRNSGEYYYYYGMDV |
| 2309 | YANG-2301 | VL domain | GAAATAGTGATGACCCAGTCTCCAGCCACCCTGTCTGTGTCTCC |
| | | nucleic | AGGGGAAAGAGCCACCCTCTCTTGCAGGGCCAGTCAGAGTGTTA |
| | | acid | ACAGCAACTTGGCCTGGTACCAGCAGAAGCCTGGCCAGGCCCCC |
| | | sequence | AGGCTCCTCATCTATGCTGTATCCACCAGGGCCACTGGTCTCCC |
| | | | AGCCAGGTTCAGTGGCAGTGGGTCTGGGACAGAATTCACTCTCA |
| | | | CCATCAGCAGCCTGCAGTCTGAAGACTTTGTAGTTTATTACTGT |
| | | | CACCAGTATAATAACTGGCCGCTCACTTTCGGCGGAGGGACCAA |
| | | | GGTGGAGATCAAA |
| 2310 | YANG-2301 | VL domain | EIVMTQSPATLSVSPGERATLSCRASQSVNSNLAWYQQKPGQAP |
| | | amino | RLLIYAVSTRATGLPARFSGSGSGTEFTLTISSLQSEDFVVYYC |
| | | acid | HQYNNWPLTFGGGTKVEIK |
| | | sequence | |
| 2311 | YANG-2301 | LCDR1 | QSVNSN |
| 2312 | YANG-2301 | LCDR2 | AVS |
| 2313 | YANG-2301 | LCDR3 | HQYNNWPLT |
| 2314 | YANG-2302 | VH domain | CAGGTCCAACTGGTACAGTCTGGGGCTGAGGTGAGGAAGCCTGG |
| | | nucleic | GGCCTCAGTGAAGGTCTCCTGCAAGGTTTCCGGATACACCCTCC |
| | | acid | CTGAATTAGCCATACACTGGGTGCGACAGGCGCCTGGAAAAGGG |
| | | sequence | CTTGAGTGGATGGGGGGTTTTATTCCTGAAGATGGTGACACAAT |
| | | | CTACGCACAGAAGTTCCAGGGCAGAGTCACCATGACCGAGGACA |
| | | | CATCTACAGACACAGCCTACATGGACCTGACCAGCCTGAGATCT |
| | | | GAGGACGCGGCCGTGTATTACTGTACATCAGGCTGGGCAGCACC |
| | | | TGGATACAATTACGGAATGGCCGTCTGGGGCCAAGGGACCACGG |
| | | | TCACCGTCTCCTCA |
| 2315 | YANG-2302 | VH domain | QVQLVQSGAEVRKPGASVKVSCKVSGYTLPELAIHWVRQAPGKG |
| | | amino | LEWMGGFIPEDGDTIYAQKFQGRVTMTEDTSTDTAYMDLTSLRS |
| | | acid | EDAAVYYCTSGWAAPGYNYGMAVWGQGTTVTVSS |
| | | sequence | |
| 2316 | YANG-2302 | HCDR1 | GYTLPELA |
| 2317 | YANG-2302 | HCDR2 | FIPEDGDT |
| 2318 | YANG-2302 | HCDR3 | TSGWAAPGYNYGMAV |
| 2319 | YANG-2302 | VL domain | GAAGTTGTGTTGACGCAGTCTCCAGGCACTTTGTCTTTGTCTTC |
| | | nucleic | AGGGGAAAGAGCCACCCTCTCCTGCAGGGCCAGTCAGAGTGTTA |
| | | acid | GCAGTAACTACTTAGCCTGGTACCAGAAGAAACCTGGCCAGGCT |
| | | sequence | CCCAGGCTCCTCATTTATGGTGCATCCAGCAGGGTCACTGGCAT |
| | | | CCCAGACAGGTTCGGTGGCAGTGGGTCTGGGACAGACTTCACTC |
| | | | TCACCATCAGCAGACTGGAGCCTGAAGATTTTGCAGTGTATTAC |
| | | | TGTCAACAATATGATGCCTCACCGTACAGTTTTGGCCAGGGGAC |
| | | | CAAGCTGGACATCAAA |
| 2320 | YANG-2302 | VL domain | EVVLTQSPGTLSLSSGERATLSCRASQSVSSNYLAWYQKKPGQA |
| | | amino | PRLLIYGASSRVTGIPDRFGGSGSGTDFTLTISRLEPEDFAVYY |
| | | acid | CQQYDASPYSFGQGTKLDIK |
| | | sequence | |
| 2321 | YANG-2302 | LCDR1 | QSVSSNY |
| 2322 | YANG-2302 | LCDR2 | GAS |
| 2323 | YANG-2302 | LCDR3 | QQYDASPYS |
| 2324 | YANG-2303 | VH domain | CAGGTCCAGTTGGTACAGTCTGGGGCTGAGGTGAAGAAGCCTGG |
| | | nucleic | GGCCTCAGTGAGGGTCTCCTGCAAGGTTTCCGGATACACCCTCC |
| | | acid | CTGAATTAGCCATACACTGGGTGCGACAGGCTCCTGGAAAAGGG |
| | | sequence | CTTGAGTGGATGGGAGGTTTTGATCCTGAGGATGGTGAGACAAT |
| | | | CTACGCACAGAAGTTCCAGGGTAGAGTCATCATGACCGAGGACA |
| | | | CATCCACAGACACAGCCTACATGGACCTGAGCAGCCTGAGATCT |
| | | | GAGGACACGGCCGTGTATTACTGTGTAAAAACCTGGGCAGCGCC |
| | | | CGGATATAATTACGGTTTGGACACCTGGGGCCAAGGGACCACGG |
| | | | TCACCGTCTCCTCA |
| 2325 | YANG-2303 | VH domain | QVQLVQSGAEVKKPGASVRVSCKVSGYTLPELAIHWVRQAPGKG |
| | | amino | LEWMGGFDPEDGETIYAQKFQGRVIMTEDTSTDTAYMDLSSLRS |
| | | acid | EDTAVYYCVKTWAAPGYNYGLDTWGQGTTVTVSS |
| | | sequence | |
| 2326 | YANG-2303 | HCDR1 | GYTLPELA |
| 2327 | YANG-2303 | HCDR2 | FDPEDGET |
| 2328 | YANG-2303 | HCDR3 | VKTWAAPGYNYGLDT |
| 2329 | YANG-2303 | VL domain | GAAATTGTGTTGACGCAGTCTCCAGGCATCCTGTCTTTGTCTCC |
| | | nucleic | AGGGGAAAGTGCCACCCTCTCCTGCAGGGCCAGTCAGAGTATTG |
| | | acid | CCAGCAACTACTTAGCCTGGTTCCAGCAGAAACCTGGCCAGGCT |
| | | sequence | CCCAGGCTCCTCATCTATGGTGCATCCAGCAGGGCCGCTGGCTT |
| | | | CCCAGACAGGTTCAGTGGCAGTGGGTCTGGGACAGACTTCACTC |
| | | | TCACCATCAAGAGACTGGAGCCTGAAGATTCTGCAGTGTATTAT |
| | | | TGTCAGCACTATGGTAGCTCACCGTGCAGTTTTGGCCAGGGGAC |
| | | | CAACCTGGAGATCAAA |
| 2330 | YANG-2303 | VL domain | EIVLTQSPGILSLSPGESATLSCRASQSIASNYLAWFQQKPGQA |
| | | amino | PRLLIYGASSRAAGFPDRFSGSGSGTDFTLTIKRLEPEDSAVYY |
| | | acid | CQHYGSSPCSFGQGTNLEIK |
| | | sequence | |
| 2331 | YANG-2303 | LCDR1 | QSIASNY |
| 2332 | YANG-2303 | LCDR2 | GAS |
| 2333 | YANG-2303 | LCDR3 | QHYGSSPCS |
| 2334 | YANG-2304 | VH domain | CAGGTCCAGCTGGTACAGTCTGGGACTGAAGTGAAGAAGCCTGG |
| | | nucleic | GGCCTCAGTGAAGGTCTCCTGCAAGGTTTCCGGATACACCCTCC |
| | | acid | CTGAATTGGCCATTCACTGGGTGCGACAGGCTCCTGGAAAAGGG |
| | | sequence | CTTGAGTGGATGGGAACTTTTGATCCTGAAGATGGTGAAACAAT |
| | | | CTGCGCACAGAAGTTCCAGGGCAGAGTCACCATGACCGAGGACA |
| | | | CATCTACAGACACAGCCTACATGGACCTGAGCAGCCTCAGATCT |
| | | | GAGGACACGGCCGTTTATTACTGTGCAACAACGTCGATTATAGC |
| | | | AGCTCGGTGGTGGTTCGACCCCTGGGGCCAGGGAACCCTGGTCA |
| | | | CCGTCTCCTCA |
| 2335 | YANG-2304 | VH domain | QVQLVQSGTEVKKPGASVKVSCKVSGYTLPELAIHWVRQAPGKG |
| | | amino | LEWMGTFDPEDGETICAQKFQGRVTMTEDTSTDTAYMDLSSLRS |
| | | acid | EDTAVYYCATTSIIAARWWFDPWGQGTLVTVSS |
| | | sequence | |
| 2336 | YANG-2304 | HCDR1 | GYTLPELA |
| 2337 | YANG-2304 | HCDR2 | FDPEDGET |
| 2338 | YANG-2304 | HCDR3 | ATTSIIAARWWFDP |
| 2339 | YANG-2304 | VL domain | CAATCTGCCCTGACTCAGCCTCCCTCCGCGTCCGGGTCTCCTGG |
| | | nucleic | ACAGTCAGTCACCATCTCCTGCACTGGAACCAGCAGTGACGTTG |
| | | acid | GTGGTTATGACTATGTCTCCTGGTACCAACAACACCCAGGCAAA |
| | | sequence | GCCCCCAAACTCATGATTTATGAGGTCAGTAAGCGGCCCTCAGG |
| | | | GGTCCCTGATCGCTTCTCTGGCTCCAAGTCTGGCAACACGGCCT |
| | | | CCCTGACCGTCTCTGGGCTCCAGGCTGAGGATGAGGCTGATTAT |
| | | | TACTGCAGTTCATTTGCAGGCAGCAACAATGTGGTTTTCGGCGG |
| | | | AGGGGCCAAGCTGACCGTCCTG |
| 2340 | YANG-2304 | VL domain | QSALTQPPSASGSPGQSVTISCTGTSSDVGGYDYVSWYQQHPGK |
| | | amino | APKLMIYEVSKRPSGVPDRFSGSKSGNTASLTVSGLQAEDEADY |
| | | acid | YCSSFAGSNNVVFGGGAKLTVL |
| | | sequence | |
| 2341 | YANG-2304 | LCDR1 | SSDVGGYDY |
| 2342 | YANG-2304 | LCDR2 | EVS |
| 2343 | YANG-2304 | LCDR3 | SSFAGSNNVV |
| 2344 | YANG-2305 | VH domain | CAGGTGCAGCTGGTGGAGTCTGGGGGAGGCGTGGTCCAGCCTGG |
| | | nucleic | GAGGTCCCTGAGACTCTCCTGTGCAGCGTCTGGATTCACCTTCA |
| | | acid | GTAGTTATGGCATGCACTGGGTCCGCCAGGCTCCAGGCAAGGGG |
| | | sequence | CTGGAGTGGGTGGCAATTATTTGGTATGATGGAAGTAAGAAATA |
| | | | CTATGCAGACTCCGTGAAGGGCCGATTCACCATCTCCAGAGACA |
| | | | ATTCCAAGAACACGCTGAATCTGCAAATGAACAGCCTGAGAGCC |
| | | | GAGGACACGGCTGTGTATTACTGTGCGAGAGAAGAGGATATAGT |
| | | | GGCTTCGGGGGGGCTCTATTACAACTTCAACGGTATGGACGTCT |
| | | | GGGGCCAAGGGACCACGGTCACCGTTTCCTCA |
| 2345 | YANG-2305 | VH domain | QVQLVESGGGVVQPGRSLRLSCAASGFTFSSYGMHWVRQAPGKG |
| | | amino | LEWVAIIWYDGSKKYYADSVKGRFTISRDNSKNTLNLQMNSLRA |
| | | acid | EDTAVYYCAREEDIVASGGLYYNFNGMDVWGQGTTVTVSS |
| | | sequence | |
| 2346 | YANG-2305 | HCDR1 | GFTFSSYG |
| 2347 | YANG-2305 | HCDR2 | IWYDGSKK |
| 2348 | YANG-2305 | HCDR3 | AREEDIVASGGLYYNFNGMDV |
| 2349 | YANG-2305 | VL domain | TCCTATGAGCTGACTCAGCCACCCTCAGTGTCCGTGTCCCCAGG |
| | | nucleic | ACAGACAGCCAGCATCACCTGCTCTGGAGATAAATTGGGGGATA |
| | | acid | AATATGCTTGCTGGTATCAGCAGAAGCCAGGCCAGTCCCCTGTG |
| | | sequence | CTGGTCATCTATCAAGATAGCAAGCGGCCCTCAGGGATCCCTGA |
| | | | GCGATTCTCTGGCTCCAACTCTGGGAACACAGCCACTCTGACCA |
| | | | TCAACGGGACCCAGGCTATGGATGAGGCTGACTATTTCTGTCAG |
| | | | GCGTGGGACAGCACCACTGTGGTATTCGGCGGAGGGACCAAGCT |
| | | | GACCGTCCTA |
| 2350 | YANG-2305 | VL domain | SYELTQPPSVSVSPGQTASITCSGDKLGDKYACWYQQKPGQSPV |
| | | amino | LVIYQDSKRPSGIPERFSGSNSGNTATLTINGTQAMDEADYFCQ |
| | | acid | AWDSTTVVFGGGTKLTVL |
| | | sequence | |
| 2351 | YANG-2305 | LCDR1 | KLGDKY |
| 2352 | YANG-2305 | LCDR2 | QDS |
| 2353 | YANG-2305 | LCDR3 | QAWDSTTVV |
| 2354 | YANG-2306 | VH domain | CAGGTCCAGCTGGTACAATCTGGGGCTGAGGTGAAGAAGCCTGG |
| | | nucleic | GGCCTCAGTGAAGGTCTCCTGCAAGGTTTCCGGATACACCCTCC |
| | | acid | CTGAAATATCCATACACTGGGTGCGACAGGCTCCTGGAAAAGGG |
| | | sequence | CTTGAGTGGATGGGAGGTTTTGATCCTGAAGATGGTGAAACAAT |
| | | | CTACGCACAGAAGTTCCAGGGCAGAGTGATCATGACCGAGGACA |
| | | | CATCTACAGACACAGCCTATATGGACCTGAGCAGCCTGAGATCT |
| | | | GAGGACACGGCCGTGTATTTCTGTGCAGCTGCCCCGGCTATAGC |
| | | | TTCAGCTTCAACCTTCTGGCTCGACCCCTGGGGCCAGGGAACCC |
| | | | TGGTCACCGTCTCCACA |
| 2355 | YANG-2306 | VH domain | QVQLVQSGAEVKKPGASVKVSCKVSGYTLPEISIHWVRQAPGKG |
| | | amino | LEWMGGFDPEDGETIYAQKFQGRVIMTEDTSTDTAYMDLSSLRS |
| | | acid | EDTAVYFCAAAPAIASASTFWLDPWGQGTLVTVST |
| | | sequence | |
| 2356 | YANG-2306 | HCDR1 | GYTLPEIS |
| 2357 | YANG-2306 | HCDR2 | FDPEDGET |
| 2358 | YANG-2306 | HCDR3 | AAAPAIASASTFWLDP |
| 2359 | YANG-2306 | VL domain | CAGTCTGCCCTGACTCAGCCTCCCTCCGCGTCCGGGTCTCCTGG |
| | | nucleic | ACAGTCAGTCACCATCTCCTGCACTGGAACCAGCAGTGACGTTG |
| | | acid | GTGGTTATAACTATGTCTCCTGGTACCAACAGCACCCAGGCAAA |
| | | sequence | GCCCCCAAACTCATGATTTATGAGGTCACTAAGCGGCCCTCAGG |
| | | | GGTCCCTGATCGCTTCTCTGGCTCCAAGTCTGACAACACGGCCT |
| | | | CCCTGACCGTCTCTGGGCTCCAGGCTGAGGATGAGGCTGATTAT |
| | | | TACTGCAGTTCATATGCAGGCAGCAACAATTTGGTATTCGGCGG |
| | | | AGGGACCAAGCTGACCGTCCTA |
| 2360 | YANG-2306 | VL domain | QSALTQPPSASGSPGQSVTISCTGTSSDVGGYNYVSWYQQHPGK |
| | | amino | APKLMIYEVTKRPSGVPDRFSGSKSDNTASLTVSGLQAEDEADY |
| | | acid | YCSSYAGSNNLVFGGGTKLTVL |
| | | sequence | |
| 2361 | YANG-2306 | LCDR1 | SSDVGGYNY |
| 2362 | YANG-2306 | LCDR2 | EVT |
| 2363 | YANG-2306 | LCDR3 | SSYAGSNNLV |

TABLE 2
constant region sequences

| | | | | |
|---|---|---|---|---|
| 417 | Human IgG1 | IGHG1*01 | Human Heavy | gcctccaccaagggcccatcggtcttccccctggcaccctcctccaagagcacctctggg |
| | constant | | Chain | ggcacagcggccctgggctgcctggtcaaggactacttccccgaaccggtgacggtgtcg |
| | region | | Constant Region | tggaactcaggcgccctgaccagcggcgtgcacaccttcccggctgtcctacagtcctca |
| | | | (IGHG1*01) | ggactctactccctcagcagcgtggtgaccgtgccctccagcagcttgggcacccagacc |
| | | | Nucleotide | tacatctgcaacgtgaatcacaagcccagcaacaccaaggtggacaagaaagttgagccc |
| | | | Sequence | aaatcttgtgacaaaactcacacatgcccaccgtgcccagcacctgaactcctgggggga |
| | | | | ccgtcagtcttcctcttccccccaaaacccaaggacaccctcatgatctcccggacccct |
| | | | | gaggtcacatgcgtggtggtggacgtgagccacgaagaccctgaggtcaagttcaactgg |
| | | | | tacgtggacggcgtggaggtgcataatgccaagacaaagccgcgggaggagcagtacaac |
| | | | | agcacgtaccgggtggtcagcgtcctcaccgtcctgcaccaggactggctgaatggcaag |
| | | | | gagtacaagtgcaaggtctccaacaaagccctcccagcccccatcgagaaaaccatctcc |
| | | | | aaagccaaagggcagccccgagaaccacaggtgtacaccctgcccccatcccgggatgag |
| | | | | ctgaccaagaaccaggtcagcctgacctgcctggtcaaaggcttctatcccagcgacatc |
| | | | | gccgtggagtgggagagcaatgggcagccggagaacaactacaagaccacgcctcccgtg |
| | | | | ctggactccgacggctccttcttcctctacagcaagctcaccgtggacaagagcaggtgg |
| | | | | cagcaggggaacgtcttctcatgctccgtgatgcatgaggctctgcacaaccactacacg |
| | | | | cagaagagcctctccctgtctccgggtaaa |
| 418 | | | Human Heavy | ASTKGPSVFPLAPSSKSTSGGTAALGCLVKDYFPEPVTVSWNSGALTSGVHTFPAVLQSS |
| | | | Chain | GLYSLSSVVTVPSSSLGTQTYICNVNHKPSNTKVDKKVEPKSCDKTHTCPPCPAPELLGG |
| | | | Constant Region | PSVFLFPPKPKDTLMISRTPEVTCVVVDVSHEDPEVKFNWYVDGVEVHNAKTKPREEQYN |
| | | | (IGHG1*01) | STYRVVSVLTVLHQDWLNGKEYKCKVSNKALPAPIEKTISKAKGQPREPQVYTLPPSRDE |
| | | | Protein | LTKNQVSLTCLVKGFYPSDIAVEWESNGQPENNYKTTPPVLDSDGSFFLYSKLTVDKSRW |
| | | | Sequence | QQGNVFSCSVMHEALHNHYTQKSLSLSPGK |
| | | | (P01857) | |
| 419 | Human IgG1 | IGHG1*02 | Human Heavy | gcctccaccaagggcccatcggtcttccccctggcaccctcctccaagagcacctctggg |
| | constant | or | Chain | ggcacagcggccctgggctgcctggtcaaggactacttccccgaaccggtgacggtgtcg |
| | region | IGHG1*05 | Constant Region | tggaactcaggcgccctgaccagcggcgtgcacaccttcccggctgtcctacagtcctca |
| | | | (IGHG1*02 | ggactctactccctcagcagcgtggtgaccgtgccctccagcagcttgggcacccagacc |
| | | | or IGHG1*05) | tacatctgcaacgtgaatcacaagcccagcaacaccaaggtggacaagaaagttgagccc |
| | | | Nucleotide | aaatcttgtgacaaaactcacacatgcccaccgtgcccagcacctgaactcctgggggga |
| | | | Sequence | ccgtcagtcttcctcttccccccaaaacccaaggacaccctcatgatctcccggacccct |
| | | | | gaggtcacatgcgtggtggtggacgtgagccacgaagaccctgaggtcaagttcaactgg |
| | | | | tacgtggacggcgtggaggtgcataatgccaagacaaagccgcgggaggagcagtacaac |
| | | | | agcacgtaccgtgtggtcagcgtcctcaccgtcctgcaccaggactggctgaatggcaag |
| | | | | gagtacaagtgcaaggtctccaacaaagccctcccagcccccatcgagaaaaccatctcc |
| | | | | aaagccaaagggcagccccgagaaccacaggtgtacaccctgcccccatcccgggatgag |
| | | | | ctgaccaagaaccaggtcagcctgacctgcctggtcaaaggcttctatcccagcgacatc |
| | | | | gccgtggagtgggagagcaatgggcagccggagaacaactacaagaccacgcctcccgtg |
| | | | | ctggactccgacggctccttcttcctctacagcaagctcaccgtggacaagagcaggtgg |
| | | | | cagcaggggaacgtcttctcatgctccgtgatgcatgaggctctgcacaaccactacacg |
| | | | | cagaagagcctctccctgtctccgggtaaa |
| 420 | | | Human Heavy Chain | A S T K G P S V F P L A P S S K S T S G G T A A L G C L V K |
| | | | Constant Region | D Y F P E P V T V S W N S G A L T S G V H T F P A V L Q S S |
| | | | (IGHG1*02) | G L Y S L S S V V T V P S S S L G T Q T Y I C N V N H K P S |
| | | | Protein | N T K V D K K V E P K S C D K T H T C P P C P A P E L L G G |
| | | | Sequence | P S V F L F P P K P K D T L M I S R T P E V T C V V V D V S |
| | | | | H E D P E V K F N W Y V D G V E V H N A K T K P R E E Q Y N |
| | | | | S T Y R V V S V L T V L H Q D W L N G K E Y K C K V S N K A |
| | | | | L P A P I E K T I S K A K G Q P R E P Q V Y T L P P S R D E |
| | | | | L T K N Q V S L T C L V K G F Y P S D I A V E W E S N G Q P |
| | | | | E N N Y K T T P P V L D S D G S F F L Y S K L T V D K S R W |
| | | | | Q Q G N V F C S S V M H E A L H N H Y T Q K S L S L S P G K |
| 421 | Human IgG1 | IGHG1*03 | Human Heavy Chain | gcctccaccaagggcccatcggtcttccccctggcaccctcctccaagagcacctctggg |
| | constant | | Constant Region | ggcacagcggccctgggctgcctggtcaaggactacttccccgaaccggtgacggtgtcg |
| | region | | (IGHG1*03) | tggaactcaggcgccctgaccagcggcgtgcacaccttcccggctgtcctacagtcctca |
| | | | Nucleotide | ggactctactccctcagcagcgtggtgaccgtgccctccagcagcttgggcacccagacc |
| | | | Sequence (Y14737) | tacatctgcaacgtgaatcacaagcccagcaacaccaaggtggacaagagagttgagccc |
| | | | | aaatcttgtgacaaaactcacacatgcccaccgtgcccagcacctgaactcctgggggga |
| | | | | ccgtcagtcttcctcttccccccaaaacccaaggacaccctcatgatctcccggacccct |
| | | | | gaggtcacatgcgtggtggtggacgtgagccacgaagaccctgaggtcaagttcaactgg |
| | | | | tacgtggacggcgtggaggtgcataatgccaagacaaagccgcgggaggagcagtacaac |
| | | | | agcacgtaccgtgtggtcagcgtcctcaccgtcctgcaccaggactggctgaatggcaag |
| | | | | gagtacaagtgcaaggtctccaacaaagccctcccagcccccatcgagaaaaccatctcc |
| | | | | aaagccaaagggcagccccgagaaccacaggtgtacaccctgcccccatcccgggaggag |
| | | | | atgaccaagaaccaggtcagcctgacctgcctggtcaaaggcttctatcccagcgacatc |
| | | | | gccgtggagtgggagagcaatgggcagccggagaacaactacaagaccacgcctcccgtg |
| | | | | ctggactccgacggctccttcttcctctatagcaagctcaccgtggacaagagcaggtgg |
| | | | | cagcaggggaacgtcttctcatgctccgtgatgcatgaggctctgcacaaccactacacg |
| | | | | cagaagagcctctccctgtccccgggtaaa |
| 422 | | | Human Heavy Chain | A S T K G P S V F P L A P S S K S T S G G T A A L G C L V K |
| | | | Constant Region | D Y F P E P V T V S W N S G A L T S G V H T F P A V L Q S S |
| | | | (IGHG1*03) | G L Y S L S S V V T V P S S S L G T Q T Y I C N V N H K P S |
| | | | Protein | N T K V D K R V E P K S C D K T H T C P P C P A P E L L G G |
| | | | Sequence | P S V F L F P P K P K D T L M I S R T P E V T C V V V D V S |
| | | | | H E D P E V K F N W Y V D G V E V H N A K T K P R E E Q Y N |
| | | | | S T Y R V V S V L T V L H Q D W L N G K E Y K C K V S N K A |
| | | | | L P A P I E K T I S K A K G Q P R E P Q V Y T L P P S R E E |
| | | | | M T K N Q V S L T C L V K G F Y P S D I A V E W E S N G Q P |
| | | | | E N N Y K T T P P V L D S D G S F F L Y S K L T V D K S R W |
| | | | | Q Q G N V F S C S V M H E A L H N H Y T Q K S L S L S P G K |
| 423 | Human IgG1 | IGHG1*04 | Human Heavy Chain | gcctccaccaagggcccatcggtcttccccctggcaccctcctccaagagcacctctggg |
| | constant | | Constant Region | ggcacagcggccctgggctgcctggtcaaggactacttccccgaaccggtgacggtgtcg |
| | region | | (IGHG1*04) | tggaactcaggcgccctgaccagcggcgtgcacaccttcccggctgtcctacagtcctca |
| | | | Nucleotide | ggactctactccctcagcagcgtggtgaccgtgccctccagcagcttgggcacccagacc |
| | | | Sequence | tacatctgcaacgtgaatcacaagcccagcaacaccaaggtggacaagaaagttgagccc |
| | | | | aaatcttgtgacaaaactcacacatgcccaccgtgcccagcacctgaactcctgggggga |
| | | | | ccgtcagtcttcctcttccccccaaaacccaaggacaccctcatgatctcccggacccct |
| | | | | gaggtcacatgcgtggtggtggacgtgagccacgaagaccctgaggtcaagttcaactgg |
| | | | | tacgtggacggcgtggaggtgcataatgccaagacaaagccgcgggaggagcagtacaac |
| | | | | agcacgtaccgtgtggtcagcgtcctcaccgtcctgcaccaggactggctgaatggcaag |
| | | | | gagtacaagtgcaaggtctccaacaaagccctcccagcccccatcgagaaaaccatctcc |
| | | | | aaagccaaagggcagccccgagaaccacaggtgtacaccctgcccccatcccgggatgag |
| | | | | ctgaccaagaaccaggtcagcctgacctgcctggtcaaaggcttctatcccagcgacatc |
| | | | | gccgtggagtgggagagcaatgggcagccggagaacaactacaagaccacgcctcccgtg |
| | | | | ctggactccgacggctccttcttcctctacagcaagctcaccgtggacaagagcaggtgg |
| | | | | cagcaggggaacatcttctcatgctccgtgatgcatgaggctctgcacaaccactacacg |
| | | | | cagaagagcctctccctgtctccgggtaaa |
| 424 | | | Human Heavy Chain | ASTKGPSVFPLAPSSKSTSGGTAALGCLVKDYFPEPVTVSWNSGALTSGVHTFPAVLQSS |
| | | | Constant Region | GLYSLSSVVTVPSSSLGTQTYICNVNHKPSNTKVDKKVEPKSCDKTHTCPPCPAPELLGG |
| | | | (IGHG1*04) | PSVFLFPPKPKDTLMISRTPEVTCVVVDVSHEDPEVKFNWYVDGVEVHNAKTKPREEQYN |
| | | | Protein | STYRVVSVLTVLHQDWLNGKEYKCKVSNKALPAPIEKTISKAKGQPREPQVYTLPPSRDE |
| | | | Sequence | LTKNQVSLTCLVKGFYPSDIAVEWESNGQPENNYKTTPPVLDSDGSFFLYSKLTVDKSRW |
| | | | | QQGNIFSCSVMHEALHNHYTQKSLSLSPGK |
| 425 | Disabled | Disabled | Disabled Human | gcctccaccaagggcccatcggtcttccccctggcaccctcctccaagagcacctctggg |
| | Human IgG1 | human | IGHG1*01 | ggcacagcggccctgggctgcctggtcaaggactacttccccgaaccggtgacggtgtcg |
| | heavy chain | IGHG1*01 | Heavy Chain | tggaactcaggcgccctgaccagcggcgtgcacaccttcccggctgtcctacagtcctca |
| | constant | | Constant | ggactctactccctcagcagcgtggtgaccgtgccctccagcagcttgggcacccagacc |
| | region | | Region | tacatctgcaacgtgaatcacaagcccagcaacaccaaggtggacaagaaagtggagccc |
| | | | Nucleotide | aaatcttgtgacaaaactcacacatgcccaccgtgcccagcacctgaactcgcgggggca |
| | | | Sequence. | ccgtcagtcttcctcttccccccaaaacccaaggacaccctcatgatctcccggacccct |
| | | | | gaggtcacatgcgtggtggtggacgtgagccacgaagaccctgaggtcaagttcaactgg |
| | | | | tacgtggacggcgtggaggtgcataatgccaagacaaagccgcgggaggagcagtacaac |
| | | | | agcacgtaccgtgtggtcagcgtcctcaccgtcctgcaccaggactggctgaatggcaag |
| | | | | gagtacaagtgcaaggtctccaacaaagccctcccagcccccatcgagaaaaccatctcc |
| | | | | aaagccaaagggcagccccgagaaccacaggtgtacaccctgcccccatcccgggatgag |
| | | | | ctgaccaagaaccaggtcagcctgacctgcctggtcaaaggcttctatcccagcgacatc |
| | | | | gccgtggagtgggagagcaatgggcagccggagaacaactacaagaccacgcctcccgtg |
| | | | | ctggactccgacggctccttcttcctctacagcaagctcaccgtggacaagagcaggtgg |
| | | | | cagcaggggaacgtcttctcatgctccgtgatgcatgaggctctgcacaaccactacacg |
| | | | | cagaagagcctctccctgtctccgggtaaa |
| 426 | | | Disabled Human | ASTKGPSVFPLAPSSKSTSGGTAALGCLVKDYFPEPVTVSWNSGALTSGVHTFPAVLQSS |
| | | | IGHG1*01 | GLYSLSSVVTVPSSSLGTQTYICNVNHKPSNTKVDKKVEPKSCDKTHTCPPCPAPELAGA |
| | | | Heavy Chain | PSVFLFPPKPKDTLMISRTPEVTCVVVDVSHEDPEVKFNWYVDGVEVHNAKTKPREEQYN |
| | | | Constant | STYRVVSVLTVLHQDWLNGKEYKCKVSNKALPAPIEKTISKAKGQPREPQVYTLPPSRDE |
| | | | Region Amino | LTKNQVSLTCLVKGFYPSDIAVEWESNGQPENNYKTTPPVLDSDGSFFLYSKLTVDKSRW |
| | | | Acid Sequence. | QQGNVFSCSVMHEALHNHYTQKSLSLSPGK |
| | | | Two residues | |
| | | | that differ | |
| | | | from the wild- | |
| | | | type sequence are | |
| | | | identified in bold. | |
| 427 | Human IgG2 | IGHG2*01 | Human Heavy Chain | gcctccaccaagggcccatcggtcttccccctggcgccctgctccaggagcacctccgag |
| | constant | or | Constant Region | agcacagccgccctgggctgcctggtcaaggactacttccccgaaccggtgacggtgtcg |
| | region | IGHG2*04 | (IGHG2*01or | tggaactcaggcgctctgaccagcggcgtgcacaccttcccagctgtcctacagtcctca |
| | | or | IGHG2*03 or | ggactctactccctcagcagcgtggtgaccgtgccctccagcaacttcggcacccagacc |
| | | IGHG2*05 | IGHG2*05) | tacacctgcaacgtagatcacaagcccagcaacaccaaggtggacaagacagttgagcgc |
| | | | Nucleotide | aaatgttgtgtcgagtgcccaccgtgcccagcaccacctgtggcaggaccgtcagtcttc |
| | | | Sequence | ctcttccccccaaaacccaaggacaccctcatgatctcccggacccctgaggtcacgtgc |
| | | | | gtggtggtggacgtgagccacgaagaccccgaggtccagttcaactggtacgtggacggc |
| | | | | gtggaggtgcataatgccaagacaaagccacgggaggagcagttcaacagcacgttccgt |
| | | | | gtggtcagcgtcctcaccgttgtgcaccaggactggctgaacggcaaggagtacaagtgc |
| | | | | aaggtctccaacaaaggcctcccagcccccatcgagaaaaccatctccaaaaccaaaggg |
| | | | | cagccccgagaaccacaggtgtacaccctgcccccatcccgggaggagatgaccaagaac |
| | | | | caggtcagcctgacctgcctggtcaaaggcttctaccccagcgacatcgccgtggagtgg |
| | | | | gagagcaatgggcagccggagaacaactacaagaccacacctcccatgctggactccgac |
| | | | | ggctccttcttcctctacagcaagctcaccgtggacaagagcaggtggcagcaggggaac |
| | | | | gtcttctcatgctccgtgatgcatgaggctctgcacaaccactacacgcagaagagcctc |
| | | | | tccctgtctccgggtaaa |
| 428 | | | Human Heavy Chain | ASTKGPSVFPLAPCSRSTSESTAALGCLVKDYFPEPVTVSWNSGALTSGVHTFPAVLQSS |
| | | | Constant Region | GLYSLSSVVTVPSSNFGTQTYTCNVDHKPSNTKVDKTVERKCCVECPPCPAPPVAGPSVF |
| | | | (IGHG2*01) | LFPPKPKDTLMISRTPEVTCVVVDVSHEDPEVQFNWYVDGVEVHNAKTKPREEQENSTER |
| | | | Protein | VVSVLTVVHQDWLNGKEYKCKVSNKGLPAPIEKTISKTKGQPREPQVYTLPPSREEMTKN |
| | | | Sequence | QVSLTCLVKGFYPSDIAVEWESNGQPENNYKTTPPMLDSDGSFFLYSKLTVDKSRWQQGN |
| | | | | VFSCSVMHEALHNHYTQKSLSLSPGK |
| 429 | Human IgG2 | IGHG2*02 | Human Heavy Chain | GCCTCCACCAAGGGCCCATCGGTCTTCCCCCTGGCGCCCTGCTCCAGGAGCACCTCCGAG |
| | constant | | Constant Region | AGCACAGCGGCCCTGGGCTGCCTGGTCAAGGACTACTTCCCCGAACCGGTGACGGTGTCG |
| | region | | (IGHG2*02) | TGGAACTCAGGCGCTCTGACCAGCGGCGTGCACACCTTCCCGGCTGTCCTACAGTCCTCA |
| | | | Nucleotide | GGACTCTACTCCCTCAGCAGCGTGGTGACCGTGACCTCCAGCAACTTCGGCACCCAGACC |
| | | | Sequence | TACACCTGCAACGTAGATCACAAGCCCAGCAACACCAAGGTGGACAAGACAGTTGAGCGC |
| | | | | AAATGTTGTGTCGAGTGCCCACCGTGCCCAGCACCACCTGTGGCAGGACCGTCAGTCTTC |
| | | | | CTCTTCCCCCCAAAACCCAAGGACACCCTCATGATCTCCCGGACCCCTGAGGTCACGTGC |
| | | | | GTGGTGGTGGACGTGAGCCACGAAGACCCCGAGGTCCAGTTCAACTGGTACGTGGACGGC |
| | | | | ATGGAGGTGCATAATGCCAAGACAAAGCCACGGGAGGAGCAGTTCAACAGCACGTTCCGT |
| | | | | GTGGTCAGCGTCCTCACCGTCGTGCACCAGGACTGGCTGAACGGCAAGGAGTACAAGTGC |
| | | | | AAGGTCTCCAACAAAGGCCTCCCAGCCCCCATCGAGAAAACCATCTCCAAAACCAAAGGG |
| | | | | CAGCCCCGAGAACCACAGGTGTACACCCTGCCCCCATCCCGGGAGGAGATGACCAAGAAC |
| | | | | CAGGTCAGCCTGACCTGCCTGGTCAAAGGCTTCTACCCCAGCGACATCGCCGTGGAGTGG |
| | | | | GAGAGCAATGGGCAGCCGGAGAACAACTACAAGACCACACCTCCCATGCTGGACTCCGAC |
| | | | | GGCTCCTTCTTCCTCTACAGCAAGCTCACCGTGGACAAGAGCAGGTGGCAGCAGGGGAAC |
| | | | | GTCTTCTCATGCTCCGTGATGCATGAGGCTCTGCACAACCACTACACACAGAAGAGCCTC |
| | | | | TCCCTGTCTCCGGGTAAA |
| 430 | | | Human Heavy Chain | ASTKGPSVFPLAPCSRSTSESTAALGCLVKDYFPEPVTVSWNSGALTSGVHTFPAVLOSS |
| | | | Constant Region | GLYSLSSVVTVTSSNFGTQTYTCNVDHKPSNTKVDKTVERKCCVECPPCPAPPVAGPSVF |
| | | | (IGHG2*02) | LFPPKPKDTLMISRTPEVTCVVVDVSHEDPEVQFNWYVDGMEVHNAKTKPREEQFNSTER |
| | | | Protein | VVSVLTVVHQDWLNGKEYKCKVSNKGLPAPIEKTISKTKGQPREPQVYTLPPSREEMTKN |
| | | | Sequence | QVSLTCLVKGFYPSDIAVEWESNGQPENNYKTTPPMLDSDGSFFLYSKLTVDKSRWQQGN |
| | | | | VFSCSVMHEALHNHYTQKSLSLSPGK |
| 431 | Human IgG2 | IGHG2*04 | Human Heavy Chain | gcctccaccaagggcccatcggtcttccccctggcgccctgctccaggagcacctccgag |
| | constant | | Constant Region | agcacagcggccctgggctgcctggtcaaggactacttccccgaaccggtgacggtgtcg |
| | region | | (IGHG2*04) | tggaactcaggcgctctgaccagcggcgtgcacaccttcccagctgtcctacagtcctca |
| | | | Nucleotide | ggactctactccctcagcagcgtggtgaccgtgccctccagcagcttgggcacccagacc |
| | | | Sequence | tacacctgcaacgtagatcacaagcccagcaacaccaaggtggacaagacagttgagcgc |
| | | | | aaatgttgtgtcgagtgcccaccgtgcccagcaccacctgtggcaggaccgtcagtcttc |
| | | | | ctcttccccccaaaacccaaggacaccctcatgatctcccggacccctgaggtcacgtgc |
| | | | | gtggtggtggacgtgagccacgaagaccccgaggtccagttcaactggtacgtggacggc |
| | | | | gtggaggtgcataatgccaagacaaagccacgggaggagcagttcaacagcacgttccgt |
| | | | | gtggtcagcgtcctcaccgttgtgcaccaggactggctgaacggcaaggagtacaagtgc |
| | | | | aaggtctccaacaaaggcctcccagcccccatcgagaaaaccatctccaaaaccaaaggg |
| | | | | cagccccgagaaccacaggtgtacaccctgcccccatcccgggaggagatgaccaagaac |
| | | | | caggtcagcctgacctgcctggtcaaaggcttctaccccagcgacatcgccgtggagtgg |
| | | | | gagagcaatgggcagccggagaacaactacaagaccacacctcccatgctggactccgac |
| | | | | ggctccttcttcctctacagcaagctcaccgtggacaagagcaggtggcagcaggggaac |
| | | | | gtcttctcatgctccgtgatgcatgaggctctgcacaaccactacacgcagaagagcctc |
| | | | | tccctgtctccgggtaaa |
| 432 | | | Human Heavy Chain | ASTKGPSVFPLAPCSRSTSESTAALGCLVKDYFPEPVTVSWNSGALTSGVHTFPAVLQSS |
| | | | Constant Region | GLYSLSSVVTVPSSSLGTQTYTCNVDHKPSNTKVDKTVERKCCVECPPCPAPPVAGPSVF |
| | | | (IGHG2*04) | LFPPKPKDTLMISRTPEVTCVVVDVSHEDPEVQFNWYVDGVEVHNAKTKPREEQFNSTER |
| | | | Protein | VVSVLTVVHQDWLNGKEYKCKVSNKGLPAPIEKTISKTKGQPREPQVYTLPPSREEMTKN |
| | | | Sequence | QVSLTCLVKGFYPSDIAVEWESNGQPENNYKTTPPMLDSDGSFFLYSKLTVDKSRWQQGN |
| | | | | VFSCSVMHEALHNHYTQKSLSLSPGK |
| 433 | Human IgG2 | IGHG2*06 | Human Heavy Chain | GCCTCCACCAAGGGCCCATCGGTCTTCCCCCTGGCGCCCTGCTCCAGGAGCACCTCCGAG |
| | constant | | Constant Region | AGCACAGCGGCCCTGGGCTGCCTGGTCAAGGACTACTTCCCCGAACCGGTGACGGTGTCG |
| | region | | (IGHG2*06) | TGGAACTCAGGCGCTCTGACCAGCGGCGTGCACACCTTCCCGGCTGTCCTACAGTCCTCA |
| | | | Nucleotide | GGACTCTACTCCCTCAGCAGCGTGGTGACCGTGCCCTCCAGCAACTTCGGCACCCAGACC |
| | | | Sequence | TACACCTGCAACGTAGATCACAAGCCCAGCAACACCAAGGTGGACAAGACAGTTGAGCGC |
| | | | | AAATGTTGTGTCGAGTGCCCACCGTGCCCAGCACCACCTGTGGCAGGACCGTCAGTCTTC |
| | | | | CTCTTCCCCCCAAAACCCAAGGACACCCTCATGATCTCCCGGACCCCTGAGGTCACGTGC |
| | | | | GTGGTGGTGGACGTGAGCCACGAAGACCCCGAGGTCCAGTTCAACTGGTACGTGGACGGC |
| | | | | GTGGAGGTGCATAATGCCAAGACAAAGCCACGGGAGGAGCAGTTCAACAGCACGTTCCGT |
| | | | | GTGGTCAGCGTCCTCACCGTCGTGCACCAGGACTGGCTGAACGGCAAGGAGTACAAGTGC |
| | | | | AAGGTCTCCAACAAAGGCCTCCCAGCCCCCATCGAGAAAACCATCTCCAAAACCAAAGGG |
| | | | | CAGCCCCGAGAACCACAGGTGTACACCCTGCCCCCATCCCGGGAGGAGATGACCAAGAAC |
| | | | | CAGGTCAGCCTGACCTGCCTGGTCAAAGGCTTCTACCCCAGCGACATCTCCGTGGAGTGG |
| | | | | GAGAGCAATGGGCAGCCGGAGAACAACTACAAGACCACACCTCCCATGCTGGACTCCGAC |
| | | | | GGCTCCTTCTTCCTCTACAGCAAGCTCACCGTGGACAAGAGCAGGTGGCAGCAGGGGAAC |
| | | | | GTCTTCTCATGCTCCGTGATGCATGAGGCTCTGCACAACCACTACACACAGAAGAGCCTC |
| | | | | TCCCTGTCTCCGGGTAAA |
| 434 | | | Human Heavy Chain | ASTKGPSVFPLAPCSRSTSESTAALGCLVKDYFPEPVTVSWNSGALTSGVHTFPAVLQSS |
| | | | Constant Region | GLYSLSSVVTVPSSNFGTQTYTCNVDHKPSNTKVDKTVERKCCVECPPCPAPPVAGPSVF |
| | | | (IGHG2*06) | LFPPKPKDTLMISRTPEVTCVVVDVSHEDPEVQFNWYVDGVEVHNAKTKPREEQFNSTER |
| | | | Protein | VVSVLTVVHQDWLNGKEYKCKVSNKGLPAPIEKTISKTKGQPREPQVYTLPPSREEMTKN |
| | | | Sequence | QVSLTCLVKGFYPSDISVEWESNGQPENNYKTTPPMLDSDGSFFLYSKLTVDKSRWQQGN |
| | | | | VFSCSVMHEALHNHYTQKSLSLSPGK |
| 435 | Human IgG4 | IGHG4*01 | Human Heavy Chain | gcttccaccaagggcccatccgtcttccccctggcgccctgctccaggagcacctccgag |
| | constant | or | Constant Region | agcacagccgccctgggctgcctggtcaaggactacttccccgaaccggtgacggtgtcg |
| | region | IGHG4*04 | (IGHG4*01 or | tggaactcaggcgccctgaccagcggcgtgcacaccttcccggctgtcctacagtcctca |
| | | | IGHG4*04) | ggactctactccctcagcagcgtggtgaccgtgccctccagcagcttgggcacgaagacc |
| | | | Nucleotide | tacacctgcaacgtagatcacaagcccagcaacaccaaggtggacaagagagttgagtcc |
| | | | Sequence | aaatatggtcccccatgcccatcatgcccagcacctgagttcctggggggaccatcagtc |
| | | | | ttcctgttccccccaaaacccaaggacactctcatgatctcccggacccctgaggtcacg |
| | | | | tgcgtggtggtggacgtgagccaggaagaccccgaggtccagttcaactggtacgtggat |
| | | | | ggcgtggaggtgcataatgccaagacaaagccgcgggaggagcagttcaacagcacgtac |
| | | | | cgtgtggtcagcgtcctcaccgtcctgcaccaggactggctgaacggcaaggagtacaag |
| | | | | tgcaaggtctccaacaaaggcctcccgtcctccatcgagaaaaccatctccaaagccaaa |
| | | | | gggcagccccgagagccacaggtgtacaccctgcccccatcccaggaggagatgaccaag |
| | | | | aaccaggtcagcctgacctgcctggtcaaaggcttctaccccagcgacatcgccgtggag |
| | | | | tgggagagcaatgggcagccggagaacaactacaagaccacgcctcccgtgctggactcc |
| | | | | gacggctccttcttcctctacagcaggctaaccgtggacaagagcaggtggcaggagggg |
| | | | | aatgtcttctcatgctccgtgatgcatgaggctctgcacaaccactacacacagaagagc |
| | | | | ctctccctgtctctgggtaaa |
| 436 | | | Human Heavy Chain | ASTKGPSVFPLAPCSRSTSESTAALGCLVKDYFPEPVTVSWNSGALTSGVHTFPAVLOSS |
| | | | Constant Region | GLYSLSSVVTVPSSSLGTKTYTCNVDHKPSNTKVDKRVESKYGPPCPSCPAPEFLGGPSV |
| | | | (IGHG4*01) | FLFPPKPKDTLMISRTPEVTCVVVDVSQEDPEVQFNWYVDGVEVHNAKTKPREEQFNSTY |
| | | | Protein | RVVSVLTVLHQDWLNGKEYKCKVSNKGLPSSIEKTISKAKGQPREPQVYTLPPSQEEMTK |
| | | | Sequence (P01861) | NQVSLTCLVKGFYPSDIAVEWESNGQPENNYKTTPPVLDSDGSFFLYSRLTVDKSRWQEG |
| | | | | NVFSCSVMHEALHNHYTQKSLSLSLGK |
| 437 | Human IgG4 | IGHG4*02 | Human Heavy Chain | gcttccaccaagggcccatccgtcttccccctggcgccctgctccaggagcacctccgag |
| | constant | | Constant Region | agcacagccgccctgggctgcctggtcaaggactacttccccgaaccggtgacggtgtcg |
| | region | | (IGHG4*02) | tggaactcaggcgccctgaccagcggcgtgcacaccttcccggctgtcctacagtcctca |
| | | | Nucleotide | ggactctactccctcagcagcgtggtgaccgtgccctccagcagcttgggcacgaagacc |
| | | | Sequence | tacacctgcaacgtagatcacaagcccagcaacaccaaggtggacaagagagttgagtcc |
| | | | | aaatatggtcccccgtgcccatcatgcccagcacctgagttcctggggggaccatcagtc |
| | | | | ttcctgttccccccaaaacccaaggacactctcatgatctcccggacccctgaggtcacg |
| | | | | tgcgtggtggtggacgtgagccaggaagaccccgaggtccagttcaactggtacgtggat |
| | | | | ggcgtggaggtgcataatgccaagacaaagccgcgggaggagcagttcaacagcacgtac |
| | | | | cgtgtggtcagcgtcctcaccgtcgtgcaccaggactggctgaacggcaaggagtacaag |
| | | | | tgcaaggtctccaacaaaggcctcccgtcctccatcgagaaaaccatctccaaagccaaa |
| | | | | gggcagccccgagagccacaggtgtacaccctgcccccatcccaggaggagatgaccaag |
| | | | | aaccaggtcagcctgacctgcctggtcaaaggcttctaccccagcgacatcgccgtggag |
| | | | | tgggagagcaatgggcagccggagaacaactacaagaccacgcctcccgtgctggactcc |
| | | | | gacggctccttcttcctctacagcaggctaaccgtggacaagagcaggtggcaggagggg |
| | | | | aatgtcttctcatgctccgtgatgcatgaggctctgcacaaccactacacgcagaagagc |
| | | | | ctctccctgtctctgggtaaa |
| 438 | | | Human Heavy Chain | ASTKGPSVFPLAPCSRSTSESTAALGCLVKDYFPEPVTVSWNSGALTSGVHTFPAVLQSS |
| | | | Constant Region | GLYSLSSVVTVPSSSLGTKTYTCNVDHKPSNTKVDKRVESKYGPPCPSCPAPEFLGGPSV |
| | | | (IGHG4*02) | FLFPPKPKDTLMISRTPEVTCVVVDVSQEDPEVQFNWYVDGVEVHNAKTKPREEQFNSTY |
| | | | Protein | RVVSVLTVVHQDWLNGKEYKCKVSNKGLPSSIEKTISKAKGQPREPQVYTLPPSQEEMTK |
| | | | Sequence | NQVSLTCLVKGFYPSDIAVEWESNGQPENNYKTTPPVLDSDGSFFLYSRLTVDKSRWQEG |
| | | | | NVFSCSVMHEALHNHYTQKSLSLSLGK |
| 439 | Human IgG4 | IGHG4*03 | Human Heavy Chain | gcttccaccaagggcccatccgtcttccccctggcgccctgctccaggagcacctccgag |
| | constant | | Constant Region | agcacagccgccctgggctgcctggtcaaggactacttccccgaaccggtgacggtgtcg |
| | region | | (IGHG4*03) | tggaactcaggcgccctgaccagcggcgtgcacaccttcccggctgtcctacagtcctca |
| | | | Nucleotide | ggactctactccctcagcagcgtggtgaccgtgccctccagcagcttgggcacgaagacc |
| | | | Sequence | tacacctgcaacgtagatcacaagcccagcaacaccaaggtggacaagagagttgagtcc |
| | | | | aaatatggtcccccatgcccatcatgcccagcacctgagttcctggggggaccatcagtc |
| | | | | ttcctgttccccccaaaacccaaggacactctcatgatctcccggacccctgaggtcacg |
| | | | | tgcgtggtggtggacgtgagccaggaagaccccgaggtccagttcaactggtacgtggat |
| | | | | ggcgtggaggtgcataatgccaagacaaagccgcgggaggagcagttcaacagcacgtac |
| | | | | cgtgtggtcagcgtcctcaccgtcctgcaccaggactggctgaacggcaaggagtacaag |
| | | | | tgcaaggtctccaacaaaggcctcccgtcctccatcgagaaaaccatctccaaagccaaa |
| | | | | gggcagccccgagagccacaggtgtacaccctgcccccatcccaggaggagatgaccaag |
| | | | | aaccaggtcagcctgacctgcctggtcaaaggcttctaccccagcgacatcgccgtggag |
| | | | | tgggagagcaatgggcagccggagaacaactacaagaccacgcctcccgtgctggactcc |
| | | | | gacggctccttcttcctctacagcaagctcaccgtggacaagagcaggtggcaggagggg |
| | | | | aacgtcttctcatgctccgtgatgcatgaggctctgcacaaccactacacgcagaagagc |
| | | | | ctctccctgtctctgggtaaa |
| 440 | | | Human Heavy Chain | ASTKGPSVFPLAPCSRSTSESTAALGCLVKDYFPEPVTVSWNSGALTSGVHTFPAVLQSS |
| | | | Constant Region | GLYSLSSVVTVPSSSLGTKTYTCNVDHKPSNTKVDKRVESKYGPPCPSCPAPEFLGGPSV |
| | | | (IGHG4*03) | FLFPPKPKDTLMISRTPEVTCVVVDVSQEDPEVQFNWYVDGVEVHNAKTKPREEQFNSTY |
| | | | Protein | RVVSVLTVLHQDWLNGKEYKCKVSNKGLPSSIEKTISKAKGQPREPQVYTLPPSQEEMTK |
| | | | Sequence | NQVSLTCLVKGFYPSDIAVEWESNGQPENNYKTTPPVLDSDGSFFLYSKLTVDKSRWQEG |
| | | | | NVFSCSVMHEALHNHYTQKSLSLSLGK |
| 441 | Human IgG4- | IGHG4- PE | Human Heavy Chain | gcctccaccaagggcccatccgtcttccccctggcgccctgctccaggagcacctccgag |
| | PE constant | | Constant Region | agcacggccgccctgggctgcctggtcaaggactacttccccgaaccagtgacggtgtcg |
| | region | | (IGHG4-PE) | tggaactcaggcgccctgaccagcggcgtgcacaccttcccggctgtcctacagtcctca |
| | | | Nucleotide | ggactctactccctcagcagcgtggtgaccgtgccctccagcagcttgggcacgaagacc |
| | | | Sequence | tacacctgcaacgtagatcacaagcccagcaacaccaaggtggacaagagagttgagtcc |
| | | | Version A | aaatatggtcccccatgcccaccatgcccagcgcctgaatttgaggggggaccatcagtc |
| | | | | ttcctgttccccccaaaacccaaggacactctcatgatctcccggacccctgaggtcacg |
| | | | | tgcgtggtggtggacgtgagccaggaagaccccgaggtccagttcaactggtacgtggat |
| | | | | ggcgtggaggtgcataatgccaagacaaagccgcgggaggagcagttcaacagcacgtac |
| | | | | cgtgtggtcagcgtcctcaccgtcctgcaccaggactggctgaacggcaaggagtacaag |
| | | | | tgcaaggtctccaacaaaggcctcccgtcatcgatcgagaaaaccatctccaaagccaaa |
| | | | | gggcagccccgagagccacaggtgtacaccctgcccccatcccaggaggagatgaccaag |
| | | | | aaccaggtcagcctgacctgcctggtcaaaggcttctaccccagcgacatcgccgtggag |
| | | | | tgggagagcaatgggcagccggagaacaactacaagaccacgcctcccgtgctggactcc |
| | | | | gacggatccttcttcctctacagcaggctaaccgtggacaagagcaggtggcaggagggg |
| | | | | aatgtcttctcatgctccgtgatgcatgaggctctgcacaaccactacacacagaagagc |
| | | | | ctctccctgtctctgggtaaa |
| 442 | | | Human Heavy Chain | gcctccaccaagggacctagcgtgttccctctcgccccctgttccaggtccacaagcgag |
| | | | Constant Region | tccaccgctgccctcggctgtctggtgaaagactactttcccgagcccgtgaccgtctcc |
| | | | (IGHG4-PE) | tggaatagcggagccctgacctccggcgtgcacacatttcccgccgtgctgcagagcagc |
| | | | Nucleotide | ggactgtatagcctgagcagcgtggtgaccgtgcccagctccagcctcggcaccaaaacc |
| | | | Sequence | tacacctgcaacgtggaccacaagccctccaacaccaaggtggacaagcgggtggagagc |
| | | | Version B | aagtacggccccccttgccctccttgtcctgcccctgagttcgagggaggaccctccgtg |
| | | | | ttcctgtttccccccaaacccaaggacaccctgatgatctcccggacacccgaggtgacc |
| | | | | tgtgtggtcgtggacgtcagccaggaggaccccgaggtgcagttcaactggtatgtggac |
| | | | | ggcgtggaggtgcacaatgccaaaaccaagcccagggaggagcagttcaattccacctac |
| | | | | agggtggtgagcgtgctgaccgtcctgcatcaggattggctgaacggcaaggagtacaag |
| | | | | tgcaaggtgtccaacaagggactgcccagctccatcgagaagaccatcagcaaggctaag |
| | | | | ggccagccgagggagccccaggtgtataccctgcctcctagccaggaagagatgaccaag |
| | | | | aaccaagtgtccctgacctgcctggtgaagggattctacccctccgacatcgccgtggag |
| | | | | tgggagagcaatggccagcccgagaacaactacaaaacaacccctcccgtgctcgatagc |
| | | | | gacggcagcttctttctctacagccggctgacagtggacaagagcaggtggcaggagggc |
| | | | | aacgtgttctcctgttccgtgatgcacgaggccctgcacaatcactacacccagaagagc |
| | | | | ctctccctgtccctgggcaag |
| 443 | Human IgG4- | IGHG4- PE | Human Heavy Chain | gccagcaccaagggcccttccgtgttccccctggccccttgcagcaggagcacctccgaa |
| | PE constant | Inacti- | Constant Region | tccacagctgccctgggctgtctggtgaaggactactttcccgagcccgtgaccgtgagc |
| | region | vated | (IGHG4-PE) | tggaacagcggcgctctgacatccggcgtccacacctttcctgccgtcctgcagtcctcc |
| | Inactivated | IGHG4 | Nucleotide | ggcctctactccctgtcctccgtggtgaccgtgcctagctcctccctcggcaccaagacc |
| | Human IgG4 | | Sequence | tacacctgtaacgtggaccacaaaccctccaacaccaaggtggacaaacgggtcgagagc |
| | constant | | Version C | aagtacggccctccctgccctccttgtcctgcccccgagttcgaaggcggacccagcgtg |
| | region | | | ttcctgttccctcctaagcccaaggacaccctcatgatcagccggacacccgaggtgacc |
| | | | | tgcgtggtggtggatgtgagccaggaggaccctgaggtccagttcaactggtatgtggat |
| | | | | ggcgtggaggtgcacaacgccaagacaaagccccgggaagagcagttcaactccacctac |
| | | | | agggtggtcagcgtgctgaccgtgctgcatcaggactggctgaacggcaaggagtacaag |
| | | | | tgcaaggtcagcaataagggactgcccagcagcatcgagaagaccatctccaaggctaaa |
| | | | | ggccagccccgggaacctcaggtgtacaccctgcctcccagccaggaggagatgaccaag |
| | | | | aaccaggtgagcctgacctgcctggtgaagggattctacccttccgacatcgccgtggag |
| | | | | tgggagtccaacggccagcccgagaacaattataagaccacccctcccgtcctcgacagc |
| | | | | gacggatccttctttctgtactccaggctgaccgtggataagtccaggtggcaggaaggc |
| | | | | aacgtgttcagctgctccgtgatgcacgaggccctgcacaatcactacacccagaagtcc |
| | | | | ctgagcctgtccctgggaaag |
| 444 | | | Human Heavy Chain | ASTKGPSVFPLAPCSRSTSESTAALGCLVKDYFPEPVTVSWNSGALTSGVHTFPAVLQSS |
| | | | Constant Region | GLYSLSSVVTVPSSSLGTKTYTCNVDHKPSNTKVDKRVESKYGPPCPPCPAPEFEGGPSV |
| | | | (IGHG4-PE) | FLFPPKPKDTLMISRTPEVTCVVVDVSQEDPEVQFNWYVDGVEVHNAKTKPREEQFNSTY |
| | | | Protein Sequence | RVVSVLTVLHQDWLNGKEYKCKVSNKGLPSSIEKTISKAKGQPREPQVYTLPPSQEEMTK |
| | | | (Amino acid | NQVSLTCLVKGFYPSDIAVEWESNGQPENNYKTTPPVLDSDGSFFLYSRLTVDKSRWQEG |
| | | | substitution | NVFSCSVMHEALHNHYTQKSLSLSLGK |
| | | | shown in BOLD) | |
| 445 | | | Inactivated | gcctccaccaagggcccatccgtcttccccctggcgccctgctccaggagcacctccgag |
| | | | Human Heavy | agcacggccgccctgggctgcctggtcaaggactacttccccgaaccagtgacggtgtcg |
| | | | Chain Constant | tggaactcaggcgccctgaccagcggcgtgcacaccttcccggctgtcctacagtcctca |
| | | | Region (IGHG4) | ggactctactccctcagcagcgtggtgaccgtgccctccagcagcttgggcacgaagacc |
| | | | Nucleotide | tacacctgcaacgtagatcacaagcccagcaacaccaaggtggacaagagagttgagtcc |
| | | | Sequence | aaatatggtcccccatgcccaccatgcccagcgcctccagttgcggggggaccatcagtc |
| | | | | ttcctgttccccccaaaacccaaggacactctcatgatctcccggacccctgaggtcacg |
| | | | | tgcgtggtggtggacgtgagccaggaagaccccgaggtccagttcaactggtacgtggat |
| | | | | ggcgtggaggtgcataatgccaagacaaagccgcgggaggagcagttcaacagcacgtac |
| | | | | cgtgtggtcagcgtcctcaccgtcctgcaccaggactggctgaacggcaaggagtacaag |
| | | | | tgcaaggtctccaacaaaggcctcccgtcatcgatcgagaaaaccatctccaaagccaaa |
| | | | | gggcagccccgagagccacaggtgtacaccctgcccccatcccaggaggagatgaccaag |
| | | | | aaccaggtcagcctgacctgcctggtcaaaggcttctaccccagcgacatcgccgtggag |
| | | | | tgggagagcaatgggcagccggagaacaactacaagaccacgcctcccgtgctggactcc |
| | | | | gacggatccttcttcctctacagcaggctaaccgtggacaagagcaggtggcaggagggg |
| | | | | aatgtcttctcatgctccgtgatgcatgaggctctgcacaaccactacacacagaagagc |
| | | | | ctctccctgtctctgggtaaa |
| 446 | | | Inactivated Human | ASTKGPSVFPLAPCSRSTSESTAALGCLVKDYFPEPVTVSWNSGALTSGVHTFPAVLQSS |
| | | | Heavy Chain | GLYSLSSVVTVPSSSLGTKTYTCNVDHKPSNTKVDKRVESKYGPPCPPCPAPPVAGGPSV |
| | | | Constant Region | FLFPPKPKDTLMISRTPEVTCVVVDVSQEDPEVQFNWYVDGVEVHNAKTKPREEQFNSTY |
| | | | (IGHG4) Protein | RVVSVLTVLHQDWLNGKEYKCKVSNKGLPSSIEKTISKAKGQPREPQVYTLPPSQEEMTK |
| | | | Sequence | NQVSLTCLVKGFYPSDIAVEWESNGQPENNYKTTPPVLDSDGSFFLYSRLTVDKSRWQEG |
| | | | (inactivating | NVFSCSVMHEALHNHYTQKSLSLSLGK |
| | | | mutations from | |
| | | | human IgG4 | |
| | | | shown in bold) | |
| 447 | Human Cκ | IGKC*01 | Human Cκ Light | cgtacggtggccgctccctccgtgttcatcttcccaccttccgacgagcagctgaagtcc |
| | constant | | Chain | ggcaccgcttctgtcgtgtgcctgctgaacaacttctacccccgcgaggccaaggtgcag |
| | region | | Constant Region | tggaaggtggacaacgccctgcagtccggcaactcccaggaatccgtgaccgagcaggac |
| | | | (IGKC*01) | tccaaggacagcacctactccctgtcctccaccctgaccctgtccaaggccgactacgag |
| | | | Nucleotide | aagcacaaggtgtacgcctgcgaagtgacccaccagggcctgtctagccccgtgaccaag |
| | | | Sequence | tctttcaaccggggcgagtgt |
| 448 | | | Cκ Light Chain | RTVAAPSVFIFPPSDEQLKSGTASVVCLLNNFYPREAKVQWKVDNALQSGNSQESVTEQD |
| | | | Constant Region | SKDSTYSLSSTLTLSKADYEKHKVYACEVTHQGLSSPVTKSENRGEC |
| | | | (IGKC*01) Amino | |
| | | | Acid Sequence | |
| 449 | Human Cκ | IGKC*02 | Cκ Light Chain | cgaactgtggctgcaccatctgtcttcatcttcccgccatctgatgagcagttgaaatct |
| | constant | | Constant | ggaactgcctctgttgtgtgcctgctgaataacttctatcccagagaggccaaagtacag |
| | region | | Region (IGKC*02) | tggaaggtggataacgccctccaatcgggtaactcccaggagagtgtcacagagcaggag |
| | | | Nucleotide | agcaaggacagcacctacagcctcagcagcaccctgacgctgagcaaagcagactacgag |
| | | | Sequence | aaacacaaagtctacgccggcgaagtcacccatcagggcctgagctcgcccgtcacaaag |
| | | | | agcttcaacaggggagagtgt |
| 450 | | | Cκ Light Chain | RTVAAPSVFIFPPSDEQLKSGTASVVCLLNNFYPREAKVQWKVDNALQSGNSQESVTEQE |
| | | | Constant Region | SKDSTYSLSSTLTLSKADYEKHKVYAGEVTHQGLSSPVTKSENRGEC |
| | | | (IGKC*02) Amino | |
| | | | Acid Sequence | |
| 451 | Human Cκ | IGKC*03 | Cκ Light | cgaactgtggctgcaccatctgtcttcatcttcccgccatctgatgagcagttgaaatct |
| | constant | | Chain Constant | ggaactgcctctgttgtgtgcctgctgaataacttctatoccagagaggccaaagtacag |
| | region | | Region (IGKC*03) | cggaaggtggataacgccctccaatcgggtaactcccaggagagtgtcacagagcaggag |
| | | | Nucleotide | agcaaggacagcacctacagcctcagcagcaccctgacgctgagcaaagcagactacgag |
| | | | Sequence | aaacacaaagtctacgcctgcgaagtcacccatcagggcctgagctcgcccgtcacaaag |
| | | | | agcttcaacaggggagagtgt |
| 452 | | | Cκ Light Chain | RTVAAPSVFIFPPSDEQLKSGTASVVCLLNNFYPREAKVQRKVDNALQSGNSQESVTEQE |
| | | | Constant | SKDSTYSLSSTLTLSKADYEKHKVYACEVTHQGLSSPVTKSENRGEC |
| | | | Region (IGKC*03) | |
| | | | Amino Acid | |
| | | | Sequence | |
| 453 | Human Cκ | IGKC*04 | Cκ Light Chain | cgaactgtggctgcaccatctgtcttcatcttcccgccatctgatgagcagttgaaatct |
| | constant | | Constant Region | ggaactgcctctgttgtgtgcctgctgaataacttctatoccagagaggccaaagtacag |
| | region | | (IGKC*04) | tggaaggtggataacgccctccaatcgggtaactcccaggagagtgtcacagagcaggac |
| | | | Nucleotide | agcaaggacagcacctacagcctcagcagcaccctgacgctgagcaaagcagactacgag |
| | | | Sequence | aaacacaaactctacgcctgcgaagtcacccatcagggcctgagctcgcccgtcacaaag |
| | | | | agcttcaacaggggagagtgt |
| 454 | | | Cκ Light | RTVAAPSVFIFPPSDEQLKSGTASVVCLLNNFYPREAKVQWKVDNALQSGNSQESVTEQD |
| | | | Chain Constant | SKDSTYSLSSTLTLSKADYEKHKLYACEVTHQGLSSPVTKSENRGEC |
| | | | Region | |
| | | | (IGKC*04) Amino | |
| | | | Acid Sequence | |
| 455 | Human Cκ | IGKC*05 | Cκ Light | cgaactgtggctgcaccatctgtcttcatcttcccgccatctgatgagcagttgaaatct |
| | constant | | Chain Constant | ggaactgcctctgttgtgtgcctgctgaataacttctatcccagagaggccaaagtacag |
| | region | | Region (IGKC*05) | tggaaggtggataacgccctccaatcgggtaactcccaggagagtgtcacagagcaggac |
| | | | Nucleotide | agcaaggacagcacctacagcctcagcaacaccctgacgctgagcaaagcagactacgag |
| | | | Sequence | aaacacaaagtctacgcctgcgaagtcacccatcagggcctgagctcgcccgtcacaaag |
| | | | | agcttcaacaggggagagtgc |
| 456 | | | Cκ Light | RTVAAPSVFIFPPSDEQLKSGTASVVCLLNNFYPREAKVQWKVDNALQSGNSQESVTEQD |
| | | | Chain Constant | SKDSTYSLSNTLTLSKADYEKHKVYACEVTHQGLSSPVTKSENRGEC |
| | | | Region (IGKC*05) | |
| | | | Amino Acid | |
| | | | Sequence | |
| 457 | Human Cλ | IGLC1*01 | Cλ Light Chain | cccaaggccaaccccacggtcactctgttcccgccctcctctgaggagctccaagccaac |
| | constant | | Constant Region | aaggccacactagtgtgtctgatcagtgacttctacccgggagctgtgacagtggcttgg |
| | region | | (IGLC1*01) | aaggcagatggcagccccgtcaaggcgggagtggagacgaccaaaccctccaaacagagc |
| | | | Nucleotide | aacaacaagtacgcggccagcagctacctgagcctgacgcccgagcagtggaagtcccac |
| | | | Sequence | agaagctacagctgccaggtcacgcatgaagggagcaccgtggagaagacagtggcccct |
| | | | (ENST000003903- | acagaatgttca |
| | | | 21.2) | |
| 458 | | | Cλ Light Chain | PKANPTVTLFPPSSEELQANKATLVCLISDFYPGAVTVAWKADGSPVKAGVETTKPSKQS |
| | | | Constant Region | NNKYAASSYLSLTPEQWKSHRSYSCQVTHEGSTVEKTVAPTECS |
| | | | (IGLC1*01) Amino | |
| | | | Acid Sequence | |
| | | | (A0A075B6K8) | |
| 459 | Human Cλ | IGLC1*02 | Cλ Light Chain | ggtcagcccaaggccaaccccactgtcactctgttcccgccctcctctgaggagctccaa |
| | constant | | Constant | gccaacaaggccacactagtgtgtctgatcagtgacttctacccgggagctgtgacagtg |
| | region | | Region (IGLC1*02) | gcctggaaggcagatggcagccccgtcaagggggagtggagaccaccaaaccctccaaa |
| | | | Nucleotide | cagagcaacaacaagtacgcggccagcagctacctgagcctgacgcccgagcagtggaag |
| | | | Sequence | tcccacagaagctacagctgccaggtcacgcatgaagggagcaccgtggagaagacagtg |
| | | | Version A | gcccctacagaatgttca |
| 460 | | | Cλ Light Chain | ggtcagcccaaggccaaccccactgtcactctgttcccgccctcctctgaggagctccaa |
| | | | Constant | gccaacaaggccacactagtgtgtctgatcagtgacttctacccgggagctgtgacagtg |
| | | | Region (IGLC1*02) | gcctggaaggcagatggcagccccgtcaaggcgggagtggagaccaccaaaccctccaaa |
| | | | Nucleotide | cagagcaacaacaagtacgcggccagcagctacctgagcctgacgcccgagcagtggaag |
| | | | Sequence | tcccacagaagctacagctgccaggtcacgcatgaagggagcaccgtggagaagacagtg |
| | | | Version B | gcccctacagaatgttca |
| 461 | Human Cλ | IGLC1*02 | Cλ Light Chain | GQPKANPTVTLFPPSSEELQANKATLVCLISDFYPGAVTVAWKADGSPVKAGVETTKPSK |
| | constant | | Constant | QSNNKYAASSYLSLTPEQWKSHRSYSCQVTHEGSTVEKTVAPTECS |
| | region | | Region (IGLC1*02) | |
| | | | Amino | |
| | | | Acid Sequence | |
| 462 | Human Cλ | IGLC2*01 | Cλ Light Chain | ggccagcctaaggccgctccttctgtgaccctgttccccccatcctccgaggaactgcag |
| | constant | | Constant | gctaacaaggccaccctcgtgtgcctgatcagcgacttctaccctggcgccgtgaccgtg |
| | region | | Region (IGLC2*01) | gcctggaaggctgatagctctcctgtgaaggccggcgtggaaaccaccaccccttccaag |
| | | | Nucleotide | cagtccaacaacaaatacgccgcctcctcctacctgtccctgacccctgagcagtggaag |
| | | | Sequence | tcccaccggtcctacagctgccaagtgacccacgagggctccaccgtggaaaagaccgtg |
| | | | Version A | gctcctaccgagtgctcc |
| 463 | | | Cλ Light Chain | ggccagcctaaagctgcccccagcgtcaccctgtttcctccctccagcgaggagctccag |
| | | | Constant | gccaacaaggccaccctcgtgtgcctgatctccgacttctatcccggcgctgtgaccgtg |
| | | | Region (IGLC2*01) | gcttggaaagccgactccagccctgtcaaagccggcgtggagaccaccacaccctccaag |
| | | | Nucleotide | cagtccaacaacaagtacgccgcctccagctatctctccctgacccctgagcagtggaag |
| | | | Sequence | tcccaccggtcctactcctgtcaggtgacccacgagggctccaccgtggaaaagaccgtc |
| | | | Version B | gcccccaccgagtgctcc |
| 464 | Human Cλ | IGLC2*01 | Cλ Light Chain | GQPKAAPSVTLFPPSSEELQANKATLVCLISDFYPGAVTVAWKADSSPVKAGVETTTPSK |
| | constant | IGLC2*02 | Constant | QSNNKYAASSYLSLTPEQWKSHRSYSCOVTHEGSTVEKTVAPTECS |
| | region | or | Region (IGLC1*02) | |
| | | IGLC2*03 | Amino | |
| | | | Acid Sequence | |
| 465 | Human Cλ | | Cλ Light Chain | ggtcagcccaaggctgccccctcggtcactctgttcccgccctcctctgaggagcttcaa |
| | constant | | Constant | gccaacaaggccacactggtgtgtctcataagtgacttctacccgggagccgtgacagtg |
| | region | | Region (IGLC2*02 | gcctggaaggcagatagcagccccgtcaagggggagtggagaccaccacaccctccaaa |
| | | | or IGLC2*03) | caaagcaacaacaagtacgcggccagcagctatctgagcctgacgcctgagcagtggaag |
| | | | Nucleotide | tcccacagaagctacagctgccaggtcacgcatgaagggagcaccgtggagaagacagtg |
| | | | Sequence | gcccctacagaatgttca |
| 467 | | | Cλ Light Chain | GQPKAAPSVTLFPPSSEELQANKATLVCLISDFYPGAVTVAWKADSSPVKAGVETTTPSK |
| | | | Constant Region | QSNNKYAASSYLSLTPEQWKSHRSYSCQVTHEGSTVEKTVAPTECS |
| | | | (IGLC2*02) Amino | |
| | | | Acid Sequence | |
| 468 | Human CA | IGLC3*01 | Cλ Light Chain | cccaaggctgccccctcggtcactctgttcccaccctcctctgaggagcttcaagccaac |
| | constant | | Constant | aaggccacactggtgtgtctcataagtgacttctacccgggagccgtgacagttgcctgg |
| | region | | Region (IGLC3*01) | aaggcagatagcagccccgtcaaggcgggggtggagaccaccacaccctccaaacaaagc |
| | | | Nucleotide | aacaacaagtacgcggccagcagctacctgagcctgacgcctgagcagtggaagtcccac |
| | | | Sequence | aaaagctacagctgccaggtcacgcatgaagggagcaccgtggagaagacagttgcccct |
| | | | | acggaatgttca |
| 469 | | | Cλ Light Chain | PKAAPSVTLFPPSSEELQANKATLVCLISDFYPGAVTVAWKADSSPVKAGVETTTPSKQS |
| | | | Constant | NNKYAASSYLSLTPEQWKSHKSYSCQVTHEGSTVEKTVAPTECS |
| | | | Region (IGLC3*01) | |
| | | | Amino | |
| | | | Acid Sequence | |
| 470 | Human Cλ | IGLC3*02 | Cλ Light Chain | ggtcagcccaaggctgccccctcggtcactctgttcccaccctcctctgaggagcttcaa |
| | constant | | Constant Region | gccaacaaggccacactggtgtgtctcataagtgacttctacccggggccagtgacagtt |
| | region | | (IGLC3*02) | gcctggaaggcagatagcagccccgtcaagggggggggagaccaccacaccctccaaa |
| | | | Nucleotide | caaagcaacaacaagtacgcggccagcagctacctgagcctgacgcctgagcagtggaag |
| | | | Sequence | tcccacaaaagctacagctgccaggtcacgcatgaagggagcaccgtggagaagacagtg |
| | | | | gcccctacggaatgttca |
| 471 | | | Cλ Light Chain | GQPKAAPSVTLFPPSSEELQANKATLVCLISDFYPGPVTVAWKADSSPVKAGVETTTPSK |
| | | | Constant | QSNNKYAASSYLSLTPEQWKSHKSYSCQVTHEGSTVEKTVAPTECS |
| | | | Region (IGLC1*02) | |
| | | | Amino | |
| | | | Acid Sequence | |
| 472 | Human Cλ | IGLC3*03 | Cλ Light Chain | ggtcagcccaaggctgccccctcggtcactctgttcccaccctcctctgaggagcttcaa |
| | constant | | Constant | gccaacaaggccacactggtgtgtctcataagtgacttctacccgggagccgtgacagtg |
| | region | | Region (IGLC3*03) | gcctggaaggcagatagcagccccgtcaaggcgggagtggagaccaccacaccctccaaa |
| | | | Nucleotide | caaagcaacaacaagtacgcggccagcagctacctgagcctgacgcctgagcagtggaag |
| | | | Sequence | tcccacaaaagctacagctgccaggtcacgcatgaagggagcaccgtggagaagacagtg |
| | | | | gcccctacagaatgttca |
| 473 | | | Cλ Light Chain | GQPKAAPSVTLFPPSSEELQANKATLVCLISDFYPGAVTVAWKADSSPVKAGVETTTPSK |
| | | | Constant | QSNNKYAASSYLSLTPEQWKSHKSYSCQVTHEGSTVEKTVAPTECS |
| | | | Region (IGLC3*03) | |
| | | | Amino | |
| | | | Acid Sequence | |
| 474 | Human Cλ | IGLC3*04 | Cλ Light Chain | ggtcagcccaaggctgccccctcggtcactctgttcccgccctcctctgaggagcttcaa |
| | constant | | Constant | gccaacaaggccacactggtgtgtctcataagtgacttctacccgggagccgtgacagtg |
| | region | | Region (IGLC3*04) | gcctggaaggcagatagcagccccgtcaaggcgggagtggagaccaccacaccctccaaa |
| | | | Nucleotide | caaagcaacaacaagtacgcggccagcagctacctgagcctgacgcctgagcagtggaag |
| | | | Sequence | tcccacagaagctacagctgccaggtcacgcatgaagggagcaccgtggagaagacagtg |
| | | | | gcccctacagaatgttca |
| 475 | | | Cλ Light Chain | GQPKAAPSVTLFPPSSEELQANKATLVCLISDFYPGAVTVAWKADSSPVKAGVETTTPSK |
| | | | Constant | QSNNKYAASSYLSLTPEQWKSHRSYSCQVTHEGSTVEKTVAPTECS |
| | | | Region (IGLC3*04) | |
| | | | Amino | |
| | | | Acid Sequence | |
| 476 | Human Cλ | IGLC6*01 | Cλ Light Chain | ggtcagcccaaggctgccccatcggtcactctgttcccgccctcctctgaggagcttcaa |
| | constant | | Constant | gccaacaaggccacactggtgtgcctgatcagtgacttctacccgggagctgtgaaagtg |
| | region | | Region (IGLC6*01) | gcctggaaggcagatggcagccccgtcaacacgggagtggagaccaccacaccctccaaa |
| | | | Nucleotide | cagagcaacaacaagtacgcggccagcagctacctgagcctgacgcctgagcagtggaag |
| | | | Sequence | tcccacagaagctacagctgccaggtcacgcatgaagggagcaccgtggagaagacagtg |
| | | | | gcccctgcagaatgttca |
| 477 | | | Cλ Light Chain | GQPKAAPSVTLFPPSSEELQANKATLVCLISDFYPGAVKVAWKADGSPVNTGVETTTPSK |
| | | | Constant | QSNNKYAASSYLSLTPEQWKSHRSYSCQVTHEGSTVEKTVAPAECS |
| | | | Region (IGLC6*01) | |
| | | | Amino | |
| | | | Acid Sequence | |
| 478 | Human Cλ | IGLC7*01 | Cλ Light Chain | ggtcagcccaaggctgccccatcggtcactctgttcccaccctcctctgaggagcttcaa |
| | constant | or | Constant | gccaacaaggccacactggtgtgtctcgtaagtgacttctacccgggagccgtgacagtg |
| | region | IGLC7*02 | Region (IGLC7*01 | gcctggaaggcagatggcagccccgtcaaggtgggagtggagaccaccaaaccctccaaa |
| | | | or IGLC7*02) | caaagcaacaacaagtatgcggccagcagctacctgagcctgacgcccgagcagtggaag |
| | | | Nucleotide | tcccacagaagctacagctgccgggtcacgcatgaagggagcaccgtggagaagacagtg |
| | | | Sequence | gcccctgcagaatgctct |
| 479 | | | Cλ Light Chain | GQPKAAPSVTLFPPSSEELQANKATLVCLVSDFYPGAVTVAWKADGSPVKVGVETTKPSK |
| | | | Constant Region | QSNNKYAASSYLSLTPEQWKSHRSYSCRVTHEGSTVEKTVAPAECS |
| | | | (IGLC7*01) Amino | |
| | | | Acid Sequence | |
| 480 | Human Cλ | IGLC7*03 | Cλ Light Chain | GGTCAGCCCAAGGCTGCCCCCTCGGTCACTCTGTTCCCACCCTCCTCTGAGGAGCTTCAA |
| | constant | | Constant Region | GCCAACAAGGCCACACTGGTGTGTCTCGTAAGTGACTTCAACCCGGGAGCCGTGACAGTG |
| | region | | (IGLC7*03) | GCCTGGAAGGCAGATGGCAGCCCCGTCAAGGTGGGAGTGGAGACCACCAAACCCTCCAAA |
| | | | Nucleotide | CAAAGCAACAACAAGTATGCGGCCAGCAGCTACCTGAGCCTGACGCCCGAGCAGTGGAAG |
| | | | Sequence | TCCCACAGAAGCTACAGCTGCCGGGTCACGCATGAAGGGAGCACCGTGGAGAAGACAGTG |
| | | | | GCCCCTGCAGAATGCTCT |
| 481 | | | Cλ Light Chain | GQPKAAPSVTLFPPSSEELQANKATLVCLVSDENPGAVTVAWKADGSPVKVGVETTKPSK |
| | | | Constant Region | QSNNKYAASSYLSLTPEQWKSHRSYSCRVTHEGSTVEKTVAPAECS |
| | | | (IGLC7*03) Amino | |
| | | | Acid Sequence | |
| 482 | Human IgG4- | IGHG4-PE | Human Heavy | GCTTCTACCAAGGGACCCAGCGTGTTCCCTCTGGCTCCTTGCTCCAGATCCACCTCCGAG |
| | PE constant | IGHG4-PE | Chain Constant | TCTACAGCTGCTCTGGGCTGCCTGGTCAAGGACTACTTTCCTGAGCCTGTGACCGTGTCT |
| | region | | Region (IGHG4- | TGGAACTCTGGCGCTCTGACATCTGGCGTGCACACATTCCCTGCTGTGCTGCAGTCCTCC |
| | Human IgG4- | | PE) Nucleotide | GGCCTGTACTCTCTGTCCTCTGTCGTGACCGTGCCTTCCTCTAGCCTGGGCACCAAGACC |
| | PE constant | | Sequence | TACACCTGTAATGTGGACCACAAGCCTTCCAACACCAAGGTGGACAAGCGCGTGGAATCT |
| | region | | (lysine clipped) | AAGTACGGCCCTCCTTGTCCTCCATGTCCTGCTCCAGAGTTTGAAGGCGGCCCTTCCGTG |
| | | | | TTTCTGTTCCCTCCAAAGCCTAAGGACACCCTGATGATCTCTCGGACCCCTGAAGTGACC |
| | | | | TGCGTGGTGGTGGATGTGTCCCAAGAGGATCCCGAGGTGCAGTTCAATTGGTACGTGGAC |
| | | | | GGCGTGGAAGTGCACAACGCCAAGACCAAGCCTAGAGAGGAACAGTTCAACTCCACCTAC |
| | | | | AGAGTGGTGTCCGTGCTGACCGTGCTGCACCAGGATTGGCTGAACGGCAAAGAGTACAAG |
| | | | | TGCAAGGTGTCCAACAAGGGCCTGCCTAGCTCCATCGAAAAGACCATCTCCAAGGCCAAG |
| | | | | GGCCAGCCTCGAGAACCCCAGGTTTACACCCTGCCTCCAAGCCAAGAGGAAATGACCAAG |
| | | | | AACCAGGTGTCCCTGACCTGCCTCGTGAAGGGATTCTACCCCTCCGATATCGCCGTGGAA |
| | | | | TGGGAGTCTAATGGCCAGCCAGAGAACAACTACAAGACAACCCCTCCTGTGCTGGACTCC |
| | | | | GACGGCTCCTTCTTTCTGTATTCCCGCCTGACCGTGGACAAGTCCAGATGGCAAGAGGGC |
| | | | | AACGTGTTCTCCTGCAGCGTGATGCACGAGGCCCTGCACAATCACTACACCCAGAAGTCC |
| | | | | CTGTCTCTGTCCCTGGGC |
| 483 | | | Human Heavy | ASTKGPSVFPLAPCSRSTSESTAALGCLVKDYFPEPVTVSWNSGALTSGVHTFPAVLQSS |
| | | | Chain Constant | GLYSLSSVVTVPSSSLGTKTYTCNVDHKPSNTKVDKRVESKYGPPCPPCPAPEFEGGPSV |
| | | | Region (IGHG4- | FLFPPKPKDTLMISRTPEVTCVVVDVSQEDPEVQFNWYVDGVEVHNAKTKPREEQFNSTY |
| | | | PE) Protein | RVVSVLTVLHQDWLNGKEYKCKVSNKGLPSSIEKTISKAKGQPREPQVYTLPPSQEEMTK |
| | | | Sequence | NQVSLTCLVKGFYPSDIAVEWESNGQPENNYKTTPPVLDSDGSFFLYSRLTVDKSRWQEG |
| | | | (lysine clipped) | NVFSCSVMHEALHNHYTQKSLSLSLG |
| 484 | Human IGHA1 | | | ASPTSPKVFPLSLCSTQPDGNVVIACLVQGFFPQEPLSVTWSESGQGVTARNFP |
| | constant | | | PSQDASGDLYTTSSQLTLPATQCLAGKSVTCHVKHYTNPSQDVTVPCPVPSTPP |
| | region | | | TPSPSTPPTPSPSCCHPRLSLHRPALEDLLLGSEANLTCTLTGLRDASGVTFTW |
| | | | | TPSSGKSAVQGPPERDLCGCYSVSSVLPGCAEPWNHGKTFTCTAAYPESKTPLT |
| | | | | ATLSKSGNTFRPEVHLLPPPSEELALNELVTLTCLARGFSPKDVLVRWLOGSQE |
| | | | | LPREKYLTWASRQEPSQGTTTFAVTSILRVAAEDWKKGDTFSCMVGHEALPLAF |
| | | | | TQKTIDRLAGKPTHVNVSVVMAEVDGTCY |
| 485 | Human | | | ASPTSPKVFPLSLDSTPQDGNVVVACLVQGFFPQEPLSVTWSESGQNVTARNFPPSQDAS |
| | IGHA2 | | | GDLYTTSSQLTLPATQCPDGKSVTCHVKHYTNSSQDVTVPCRVPPPPPCCHPRLSLHRPA |
| | constant | | | LEDLLLGSEANLTCTLTGLRDASGATFTWTPSSGKSAVQGPPERDLCGCYSVSSVLPGCA |
| | region | | | QPWNHGETFTCTAAHPELKTPLTANITKSGNTFRPEVHLLPPPSEELALNELVTLTCLAR |
| | | | | GFSPKDVLVRWLQGSQELPREKYLTWASRQEPSQGTTTYAVTSILRVAAEDWKKGETFSC |
| | | | | MVGHEALPLAFTQKTIDRMAGKPTHINVSVVMAEADGTCY |

| | heavy chain v | heavy chain j | light chain v | light chain j |
|---|---|---|---|---|
| Ab name | gene segment | gene segment | gene segment | gene segment |
| IMPI-001 | IGHV4-4*02 | IGHJ4*02 | IGKV2D-30*01 | IGKJ4*01 |
| IMPI-002 | IGHV3-53*01 | IGHJ6*02 | IGKV1-9*d01 | IGKJ4*01 |
| IMPI-003 | IGHV3-9*01 | IGHJ6*02 | IGKV1-6*01 | IGKJ1*01 |
| IMPI-004 | IGHV3-33*01 | IGHJ6*02 | IGKV3-20*01 | IGKJ4*01 |
| IMPI-005 | IGHV3-53*01 | IGHJ6*02 | IGKV1-9*d01 | IGKJ5*01 |
| IMPI-006 | IGHV4-39*01 | IGHJ6*02 | IGKV1D-16*01 | IGKJ4*01 |
| IMPI-007 | IGHV4-4*02 | IGHJ4*02 | IGKV2D-30*01 | IGKJ4*01 |
| IMPI-008 | IGHV3-9*01 | IGHJ6*02 | IGKV1D-13*d01 | IGKJ4*01 |
| IMPI-009 | IGHV5-51*01 | IGHJ4*02 | IGKV1D-13*d01 | IGKJ1*01 |
| IMPI-010 | IGHV3-9*01 | IGHJ6*02 | IGKV1D-13*d01 | IGKJ4*01 |
| IMPI-011 | IGHV5-51*01 | IGHJ4*02 | IGKV1D-13*d01 | IGKJ1*01 |
| IMPI-012 | IGHV3-53*01 | IGHJ6*02 | IGKV1-9*d01 | IGKJ4*01 |
| IMPI-013 | IGHV3-9*01 | IGHJ6*02 | IGKV1-6*01 | IGKJ1*01 |
| IMPI-014 | IGHV4-31*03 | IGHJ4*02 | IGKV1D-13*d01 | IGKJ4*01 |
| IMPI-015 | IGHV5-51*01 | IGHJ4*02 | IGKV1D-13*d01 | IGKJ1*01 |
| IMPI-016 | IGHV5-51*01 | IGHJ4*02 | IGKV1D-13*d01 | IGKJ1*01 |
| IMPI-017 | IGHV3-53*01 | IGHJ6*02 | IGKV1-9*d01 | IGKJ1*01 |
| IMPI-018 | IGHV4-31*03 | IGHJ4*02 | IGKV1D-13*d01 | IGKJ4*01 |
| IMPI-019 | IGHV4-4*02 | IGHJ4*02 | IGKV2D-30*01 | IGKJ4*01 |
| IMPI-020 | IGHV4-4*02 | IGHJ4*02 | IGKV2D-30*01 | IGKJ4*01 |
| IMPI-021 | IGHV3-53*01 | IGHJ6*02 | IGKV1-9*d01 | IGKJ2*04 |
| IMPI-022 | IGHV3-9*01 | IGHJ6*02 | IGKV1D-13*d01 | IGKJ4*01 |
| IMPI-023 | IGHV4-4*02 | IGHJ4*02 | IGKV2D-30*01 | IGKJ4*01 |
| IMPI-024 | IGHV5-51*01 | IGHJ4*02 | IGKV1D-13*d01 | IGKJ1*01 |
| IMPI-025 | IGHV4-4*02 | IGHJ4*02 | IGKV2D-30*01 | IGKJ4*01 |
| IMPI-026 | IGHV5-51*01 | IGHJ4*02 | IGKV1D-13*d01 | IGKJ1*01 |
| IMPI-027 | IGHV4-31*03 | IGHJ4*02 | IGKV1D-13*d01 | IGKJ4*01 |
| IMPI-028 | IGHV3-53*01 | IGHJ4*02 | IGKV3-20*01 | IGKJ1*01 |
| IMPI-029 | IGHV3-53*01 | IGHJ6*02 | IGKV1-9*d01 | IGKJ5*01 |
| IMPI-030 | IGHV4-4*02 | IGHJ4*02 | IGKV2D-30*01 | IGKJ4*01 |
| IMPI-031 | IGHV3-9*01 | IGHJ6*02 | IGKV1D-13*d01 | IGKJ4*01 |
| IMPI-032 | IGHV4-4*02 | IGHJ4*02 | IGKV2D-30*01 | IGKJ4*01 |
| IMPI-033 | IGHV4-31*03 | IGHJ4*02 | IGKV1D-13*d01 | IGKJ4*01 |
| IMPI-034 | IGHV5-51*01 | IGHJ4*02 | IGKV1D-13*d01 | IGKJ1*01 |
| IMPI-035 | IGHV3-9*01 | IGHJ6*02 | IGKV1D-13*d01 | IGKJ4*01 |
| IMPI-036 | IGHV1-8*01 | IGHJ4*02 | IGKV6-21*01 | IGKJ3*01 |
| IMPI-037 | IGHV3-15*01 | IGHJ4*02 | IGKV1-9*d01 | IGKJ1*01 |
| IMPI-038 | IGHV3-30*18 | IGHJ6*02 | IGKV1-12*01 | IGKJ1*01 |
| IMPI-039 | IGHV4-4*02 | IGHJ4*02 | IGKV2D-30*01 | IGKJ4*01 |
| IMPI-040 | IGHV4-4*02 | IGHJ4*02 | IGKV2D-30*01 | IGKJ4*01 |
| IMPI-041 | IGHV1-8*01 | IGHJ4*02 | IGKV6-21*01 | IGKJ3*01 |
| IMPI-042 | IGHV3-53*01 | IGHJ6*02 | IGKV1-33*01 | IGKJ5*01 |
| IMPI-043 | IGHV5-51*01 | IGHJ4*02 | IGKV1D-13*d01 | IGKJ1*01 |
| IMPI-044 | IGHV5-51*01 | IGHJ4*02 | IGKV1D-13*d01 | IGKJ1*01 |
| IMPI-045 | IGHV4-31*03 | IGHJ4*02 | IGKV1D-13*d01 | IGKJ4*01 |
| IMPI-046 | IGHV5-51*01 | IGHJ4*02 | IGKV1D-13*d01 | IGKJ1*01 |
| IMPI-047 | IGHV3-53*01 | IGHJ6*02 | IGKV1-9*d01 | IGKJ4*01 |
| IMPI-048 | IGHV4-31*03 | IGHJ4*02 | IGKV1D-13*d01 | IGKJ4*01 |
| IMPI-049 | IGHV5-51*01 | IGHJ4*02 | IGKV1D-13*d01 | IGKJ1*01 |
| IMPI-050 | IGHV5-51*01 | IGHJ4*02 | IGKV1D-13*d01 | IGKJ1*01 |
| IMPI-051 | IGHV5-51*01 | IGHJ4*02 | IGKV1D-13*d01 | IGKJ1*01 |
| IMPI-052 | IGHV3-53*01 | IGHJ6*02 | IGKV1-9*d01 | IGKJ4*01 |
| IMPI-053 | IGHV4-4*02 | IGHJ4*02 | IGKV2D-30*01 | IGKJ4*01 |
| IMPI-054 | IGHV3-53*01 | IGHJ6*02 | IGKV1-33*01 | IGKJ5*01 |
| IMPI-055 | IGHV1-8*01 | IGHJ4*02 | IGKV6-21*01 | IGKJ3*01 |
| IMPI-056 | IGHV3-53*01 | IGHJ6*02 | IGKV1-9*d01 | IGKJ5*01 |
| IMPI-057 | IGHV3-9*01 | IGHJ6*02 | IGKV1D-13*d01 | IGKJ4*01 |
| IMPI-058 | IGHV5-51*01 | IGHJ4*02 | IGKV1D-13*d01 | IGKJ1*01 |
| IMPI-059 | IGHV3-53*01 | IGHJ3*02 | IGKV1-33*01 | IGKJ2*04 |
| IMPI-060 | IGHV3-53*01 | IGHJ6*02 | IGKV1-9*d01 | IGKJ2*04 |
| IMPI-061 | IGHV3-20*d01 | IGHJ4*02 | IGKV3-20*01 | IGKJ2*04 |
| IMPI-062 | IGHV3-20*d01 | IGHJ4*02 | IGKV3-20*01 | IGKJ2*04 |
| IMPI-063 | IGHV3-20*d01 | IGHJ4*02 | IGKV3-20*01 | IGKJ2*04 |
| IMPI-064 | IGHV3-20*d01 | IGHJ4*02 | IGKV3-20*01 | IGKJ2*04 |
| IMPI-065 | IGHV3-20*d01 | IGHJ4*02 | IGKV3-20*01 | IGKJ2*04 |
| IMPI-066 | IGHV3-20*d01 | IGHJ4*02 | IGKV3-20*01 | IGKJ2*04 |
| IMPI-067 | IGHV3-30*18 | IGHJ6*02 | IGKV3-20*01 | IGKJ4*01 |
| IMPI-068 | IGHV3-30*18 | IGHJ6*02 | IGKV1D-13*d01 | IGKJ3*01 |
| IMPI-069 | IGHV3-20*d01 | IGHJ4*02 | IGKV3-20*01 | IGKJ2*04 |
| IMPI-070 | IGHV3-20*d01 | IGHJ4*02 | IGKV3-20*01 | IGKJ2*04 |
| IMPI-071 | IGHV3-20*d01 | IGHJ4*02 | IGKV3-20*01 | IGKJ2*04 |
| IMPI-072 | IGHV3-30*18 | IGHJ6*02 | IGKV3-20*01 | IGKJ4*01 |
| YANG-1101 | IGHV4-39*01 | IGHJ3*02 | IGLV2-23*d02 | IGLJ2*01 |
| YANG-1102 | IGHV4-4*02 | IGHJ4*02 | IGLV1-40*01 | IGLJ3*02 |
| YANG-1103 | IGHV3-53*01 | IGHJ4*02 | IGKV1-9*d01 | IGKJ5*01 |
| YANG-1105 | IGHV3-53*01 | IGHJ6*02 | IGKV1-9*d01 | IGKJ2*04 |
| YANG-1106 | IGHV3-53*01 | IGHJ4*02 | IGKV3-15*01 | IGKJ1*01 |
| YANG-1107 | IGHV3-53*01 | IGHJ4*02 | IGKV1-9*d01 | IGKJ5*01 |
| YANG-1108 | IGHV3-30*18 | IGHJ6*02 | IGKV1D-13*d01 | IGKJ4*01 |
| YANG-1109 | IGHV3-53*01 | IGHJ6*02 | IGKV1-9*d01 | IGKJ2*04 |
| YANG-1110 | IGHV3-30*18 | IGHJ6*02 | IGLV4-60*d03 | IGLJ1*01 |
| YANG-1111 | IGHV3-48*02 | IGHJ4*02 | IGLV3-21*d01 | IGLJ2*01 |
| YANG-1112 | IGHV2-5*10 | IGHJ3*02 | IGLV3-10*01 | IGLJ2*01 |
| YANG-1113 | IGHV3-21*03 | IGHJ4*02 | IGLV2-14*01 | IGLJ2*01 |
| YANG-1114 | IGHV3-21*03 | IGHJ4*02 | IGLV2-14*01 | IGLJ2*01 |
| YANG-1115 | IGHV3-21*03 | IGHJ4*02 | IGLV2-14*01 | IGLJ2*01 |
| YANG-1116 | IGHV3-21*03 | IGHJ4*02 | IGLV2-14*01 | IGLJ2*01 |
| YANG-1117 | IGHV3-21*03 | IGHJ4*02 | IGLV2-14*01 | IGLJ2*01 |
| YANG-1118 | IGHV3-21*03 | IGHJ4*02 | IGLV2-14*01 | IGLJ3*02 |
| YANG-1119 | IGHV3-21*03 | IGHJ4*02 | IGLV2-14*01 | IGLJ2*01 |
| YANG-1201 | IGHV4-4*02 | IGHJ6*02 | IGKV3-20*01 | IGKJ1*01 |
| YANG-1202 | IGHV3-7*01 | IGHJ6*02 | IGKV1-16*02 | IGKJ1*01 |
| YANG-1203 | IGHV3-9*01 | IGHJ6*02 | IGKV1-33*01 | IGKJ3*01 |
| YANG-1204 | IGHV3-30*18 | IGHJ3*02 | IGKV2-30*01 | IGKJ4*01 |
| YANG-1205 | IGHV1-46*03 | IGHJ4*02 | IGKV2D-29*01 | IGKJ1*01 |
| YANG-1206 | IGHV3-30*18 | IGHJ4*02 | IGLV1-40*01 | IGLJ3*02 |
| YANG-1207 | IGHV3-33*01 | IGHJ4*02 | IGLV3-1*01 | IGLJ2*01 |
| YANG-1301 | IGHV3-33*01 | IGHJ5*02 | IGLV3-1*01 | IGLJ2*01 |
| YANG-1302 | IGHV3-33*01 | IGHJ5*02 | IGLV3-1*01 | IGLJ2*01 |
| YANG-1303 | IGHV1-18*01 | IGHJ4*02 | IGLV3-10*01 | IGLJ3*02 |
| YANG-1304 | IGHV4-34*01 | IGHJ4*02 | IGLV3-10*01 | IGLJ3*02 |
| YANG-1305 | IGHV3-30*18 | IGHJ6*02 | IGKV1-27*01 | IGKJ3*01 |
| YANG-1401 | IGHV4-4*02 | IGHJ5*02 | IGKV1D-16*01 | IGKJ4*01 |
| YANG-1402 | IGHV3-53*01 | IGHJ6*02 | IGKV1-9*d01 | IGKJ4*01 |
| YANG-1403 | IGHV3-33*01 | IGHJ6*02 | IGKV1D-17*01 | IGKJ1*01 |
| YANG-2101 | IGHV3-13*01 | IGHJ4*02 | IGKV1-16*02 | IGKJ4*01 |
| YANG-2102 | IGHV3-13*01 | IGHJ6*02 | IGKV1-16*02 | IGKJ1*01 |
| YANG-2103 | IGHV3-53*01 | IGHJ6*02 | IGLV3-21*d01 | IGLJ1*01 |
| YANG-2104 | IGHV3-13*01 | IGHJ6*02 | IGKV1-16*02 | IGKJ1*01 |
| YANG-2105 | IGHV3-53*01 | IGHJ6*02 | IGKV1-9*d01 | IGKJ4*01 |
| YANG-2106 | IGHV3-33*01 | IGHJ6*02 | IGKV1D-17*01 | IGKJ1*01 |
| YANG-2107 | IGHV4-4*02 | IGHJ3*02 | IGLV2-23*d02 | IGLJ2*01 |
| YANG-2108 | IGHV4-4*02 | IGHJ3*02 | IGLV2-23*d02 | IGLJ2*01 |
| YANG-2109 | IGHV1-69*05 | IGHJ6*02 | IGKV1-17*01 | IGKJ4*01 |
| YANG-2110 | IGHV3-53*01 | IGHJ4*02 | IGKV3D-7*01 | IGKJ4*01 |
| YANG-2111 | IGHV3-9*01 | IGHJ4*02 | IGKV2-28*01 | IGKJ5*01 |
| YANG-2112 | IGHV1-24*d01 | IGHJ6*02 | IGLV1-47*01 | IGLJ2*01 |
| YANG-2201 | IGHV3-30*18 | IGHJ4*02 | IGKV2D-28*d01 | IGKJ5*01 |
| YANG-2202 | IGHV3-23*04 | IGHJ4*02 | IGKV1-12*01 | IGKJ5*01 |
| YANG-2203 | IGHV1-46*03 | IGHJ3*02 | IGLV1-51*01 | IGLJ2*01 |
| YANG-2204 | IGHV1-46*03 | IGHJ3*02 | IGLV1-51*01 | IGLJ3*02 |
| YANG-2205 | IGHV1-46*03 | IGHJ3*02 | IGLV1-51*01 | IGLJ3*02 |
| YANG-2206 | IGHV1-46*03 | IGHJ3*02 | IGLV1-51*01 | IGLJ3*02 |
| YANG-2207 | IGHV1-46*03 | IGHJ3*02 | IGLV1-51*01 | IGLJ3*02 |
| YANG-2208 | IGHV1-46*03 | IGHJ3*02 | IGLV1-51*01 | IGLJ3*02 |
| YANG-2301 | IGHV1-24*d01 | IGHJ6*02 | IGKV3-15*01 | IGKJ4*01 |
| YANG-2302 | IGHV1-24*d01 | IGHJ6*02 | IGKV3-20*01 | IGKJ2*04 |
| YANG-2303 | IGHV1-24*d01 | IGHJ6*02 | IGKV3-20*01 | IGKJ2*04 |
| YANG-2304 | IGHV1-24*d01 | IGHJ5*02 | IGLV2-8*01 | IGLJ2*01 |
| YANG-2305 | IGHV3-33*01 | IGHJ6*02 | IGLV3-1*01 | IGLJ2*01 |
| YANG-2306 | IGHV1-24*d01 | IGHJ5*02 | IGLV2-8*01 | IGLJ2*01 |

1. A neutralising antibody that specifically binds to the receptor binding domain (RBD) of a SARS-CoV-2 spike protein, wherein the antibody competes for binding to the SARS-CoV-2 spike protein with a human ACE2 receptor.

2. The antibody according to claim 1, wherein the antibody comprises a variable heavy (VH) domain sequence comprising complementarity determining regions (CDRs) HCDR1, HCDR2 and HCDR3, and a variable light (VL) domain sequence comprising complementarity determining regions LCDR1, LCDR2 and LCDR3, and wherein HCDR3 is the HCDR3 of antibody IMPI-037 (SEQ ID NO: 182).

3. The antibody according to claim 2, wherein the HCDR1 is SEQ ID NO: 180, the HCDR2 is SEQ ID NO: 181, the HCDR3 is SEQ ID NO: 182, the LCDR1 is SEQ ID NO: 185, the LCDR2 is SEQ ID NO: 18, and the LCDR3 is SEQ ID NO: 186-.

4. The antibody according to claim 3, wherein the VH domain comprises SEQ ID NO: 179 and the VL domain comprises SEQ ID NO: 184, optionally wherein the VH domain sequence includes 1, 2, 3, 4 or 5 amino acid alterations outside the CDRs and optionally wherein the VL domain sequence includes 1, 2, 3, 4 or 5 amino acid alterations outside the CDRs; or wherein the VH domain comprises a sequence having at least 90% identity to SEQ ID NO: 179 and the VL domain comprises a sequence having at least 90% identity to SEQ ID NO: 184, and wherein the HCDR1 is SEQ ID NO: 180, the HCDR2 is SEQ ID NO: 181, the HCDR3 is SEQ ID NO: 182, the LCDR1 is SEQ ID NO: 185, the LCDR2 is SEQ ID NO: 18, and the LCDR3 is SEQ ID NO: 186.

5. (canceled)

6. The antibody according to claim 1, wherein the VH domain comprises SEQ ID NO: 179 and the VL domain comprises SEQ ID NO: 184.

7.-23. (canceled)

24. An antibody which (ij competes for binding to the SARS-CoV-2 spike protein with the antibody of claim 1 or (ii) binds to the same epitope on the SARS-CoV-2 spike protein as the antibody of claim 1.

25. (canceled)

26. The antibody according to claim 1, wherein:
(i) the antibody is a human IgG1 antibody, optionally wherein the antibody is a human IgG1 antibody comprising a constant region sequence of SEQ ID NO: 418;
(ii) the antibody is a human IgG4 antibody, optionally wherein the antibody is a human IgG4 antibody comprising a constant region sequence of SEQ ID NO: 436: or
(iii) the antibody comprises kappa (κ) light chain constant regions, preferably wherein the κ light chain constant regions sequence is SEO ID NO: 448.

27.-30. (canceled)

31. A nucleic acid comprising a sequence that encodes a VH domain and/or an VL domain of the antibody of claim 1.

32. A vector comprising the nucleic acid of claim 31, optionally wherein the vector is a CHO vector.

33. A host cell comprising the nucleic acid of claim 31.

34. A pharmaceutical composition comprising an antibody according to claim 1 and a pharmaceutically acceptable excipient.

35. (canceled)

36. The pharmaceutical composition according to claim 34, wherein:
the pharmaceutical composition is formulated for intravenous administration, intramuscular administration, or subcutaneous administration; and/or
the pharmaceutical composition further comprises at least one additional therapeutic agent, optionally wherein the at least one additional therapeutic agent is at least one additional antibody, optionally wherein the at least one additional antibody is selected from:
a. an antibody that specifically binds to the receptor binding domain (RBD) of the S1 subunit of the SARS-CoV-2 spike protein and competes for binding to the SARS-CoV-2 spike protein with the human ACE2 receptor;
b. an antibody that specifically binds to the receptor binding domain (RBD) of the S1 subunit of the SARS-CoV-2 spike protein and does not compete for binding to the SARS-CoV-2 spike protein with the human ACE2 receptor;
c. an antibody that specifically binds to the N-terminal domain (NTD) of the S1 subunit of the of SARS-CoV-2 spike protein;
d. an antibody that specifically binds to the S2 subunit of the of SARS-CoV-2 spike protein; and
e. an antibody that preferentially binds to the trimer form of the SARS-CoV-2 spike protein over the isolated RBD domain, S1 subunit and S2 subunit of the SARS-CoV-2 spike protein.

37.-39. (canceled)

40. A kit comprising the pharmaceutical composition of claim 34, optionally further comprising:
(i) at least one further therapeutic agent, optionally wherein the at least one further therapeutic agent is a further pharmaceutical composition comprising at least one additional antibody, optionally wherein the at least one additional antibody is selected from:
a. an antibody that specifically binds to the receptor binding domain (RBD) of the S1 subunit of the SARS-CoV-2 spike protein and competes for binding to the SARS-CoV-2 spike protein with the human ACE2 receptor;
b. an antibody that specifically binds to the receptor binding domain (RBD) of the S1 subunit of the SARS-CoV-2 spike protein and does not compete for binding to the SARS-CoV-2 spike protein with the human ACE2 receptor:
c. an antibody that specifically binds to the N-terminal domain (NTD) of the S1 subunit of the of SARS-CoV-2 spike protein;
d. an antibody that specifically binds to the S2 subunit of the of SARS-CoV-2 spike protein; and
e. an antibody that preferentially binds to the trimer form of the SARS-CoV-2 spike protein over the isolated RBD domain, S1 subunit and S2 subunit of the SARS-CoV-2 spike protein; and/or
(ii) a label or instructions for use to treat and/or prevent a SARS-CoV-2-related disease or condition, such as COVID-19: optionally wherein the label or instructions comprise a marketing authorisation number: optionally wherein the kit comprises an intravenous device or an injection device that comprises the antibody.

41.-49. (canceled)

50. A method of treating or preventing a SARS-CoV-2-related disease or condition in a human subject, wherein the method comprises administering to the human subject a therapeutically effective amount of an antibody according to claim 1.

51.-55. (canceled)

56. The method of claim 50, wherein:
(1) the SARS-CoV-2 related disease or condition is COVID-19; and/or
(2) the method further comprises administering at least one additional therapeutic agent, optionally wherein:
(i) administering the at least one additional therapeutic agent is simultaneous, separate or sequential to administering of the antibody;
(ii) the at least one additional therapeutic agent is at least one additional antibody, optionally wherein the at least one additional antibody is selected from:
a. an antibody that specifically binds to the receptor binding domain (RBD) of the S1 subunit of the SARS-CoV-2 spike protein and competes for binding to the SARS-CoV-2 spike protein with the human ACE2 receptor;
b. an antibody that specifically binds to the receptor binding domain (RBD) of the S1 subunit of the SARS-CoV-2 spike protein and does not compete for binding to the SARS-CoV-2 spike protein with the human ACE2 receptor;
c. an antibody that specifically binds to the N-terminal domain (NTD) of the S1 subunit of the of SARS-CoV-2 spike protein;
d. an antibody that specifically binds to the S2 subunit of the of SARS-CoV-2 spike protein; and
e. an antibody tha preferentially binds to the trimer form of the SARS-CoV-2 spike protein over the isolated RBD domain, S1 subunit and S2 subunit of the SARS-CoV-2 spike protein.

57. (canceled)

58. A method of determining the presence or absence of SARS-CoV-2, in a sample, the method comprising:
contacting the sample with the antibody of claim 1; and
testing for binding between the antibody and SARS-CoV-2 in the sample;
wherein detection of binding indicates the presence of SARS-CoV-2 in the sample and
wherein absence of binding indicates the absence of SARS-CoV-2 in the sample.

59. The method according to claim 58,
wherein:
(i) the antibody comprises or is conjugated to a detectable label;
(ii) the sample has been obtained from a human who has been or is suspected of having been infected with SARS-CoV-2 and/or who exhibits one or more symptoms of a SARS-CoV-2-related disease or condition, such as COVID-19; and/or
(iii) the sample is a serum sample, a plasma sample, a whole blood sample, an oral sample, a nasal swab sample, an urine sample, a faeces sample, a cerebrospinal fluid (CFS) sample, or a sample from any suspected SARS-CoV-2 infected organ or tissue.

60.-63. (canceled)

64. A diagnostic kit for use in a method of determining the presence or absence of SARS-CoV-2 comprising an antibody according to claim 1, and optionally one or more buffering solutions, optionally
(i) wherein the antibody comprises or is conjugated to a detectable label: or
(ii) comprising a first reagent comprising the antibody of claim 1 and a second reagent comprising a detector molecule that binds to the first reagent, optionally wherein the detector molecule is an antibody that comprises or is conjugated to a detectable label.

65.-72. (canceled)

73. A host cell comprising the vector of claim 32.

74. A pharmaceutical composition comprising an isolated nucleic acid encoding an antibody according to claim 1 and a pharmaceutically acceptable excipient.

75. A pharmaceutical composition comprising an isolated nucleic acid according to claim 31 and a pharmaceutically acceptable excipient.

76. A method of treating a disease in a subject, wherein the method comprises administering to the subject a therapeutically effective amount of an antibody according to claim 1.
